(19) United States
(12) Patent Application Publication
(10) Pub. No.: US 2023/0303570 A1
(43) Pub. Date: Sep. 28, 2023

(54) KINASE INHIBITORS AND USES THEREOF

(71) Applicant: ABM Therapeutics Corporation, San Diego, CA (US)

(72) Inventor: Chen CHEN, San Diego, CA (US)

(21) Appl. No.: 18/019,377

(22) PCT Filed: Aug. 6, 2021
(86) PCT No.: PCT/US2021/044907

§ 371 Date: Feb. 2, 2023

Related U.S. Application Data
(60) Provisional application No. 63/063,113, filed on Aug. 7, 2020.

(51) Int. Cl. C07D 471/14; A61P 35/00

(52) U.S. Cl. CPC C07D 471/14; A61P 35/00

(57) ABSTRACT

Provided are cyclic iminopyridimdine compounds and their bicyclic derivatives, pharmaceutical compositions comprising such compounds, and methods of using such compounds or compositions, such as methods of treating a proliferation disorder, such as a cancer or a tumor, or in some embodiments disease or disorders related to the dysregulation of kinase such as, but not limited to kinases such as MAPK, PDGFR, Src, PAKs, c-Kit, EphA2, EphB4, FGFR, Axl, and c-Met.

### CROSS-REFERENCE TO RELATED APPLICATION

This application claims priority to U.S. Provisional Application No. 63/063,113, filed Aug. 7, 2020, the contents of which are incorporated by reference in their entirety.

### FIELD

The present disclosure relates to compounds, pharmaceutical compositions comprising such compounds, and use of such compounds or compositions in methods of treatment or in medicaments for treatment of a proliferation disorder, a cancer or a tumor, or in some embodiments diseases or disorders related to the dysregulation of kinase such as, but not limited to MAPK, PDGFR, Src, PAKs, c-Kit, EphA2, EphB4, FGFR, Axl, and c-Met kinase.

### BACKGROUND

The present disclosure relates to the treatment of abnormal cell growth in mammals especially humans, such as cancer and, more specifically brain cancer, with novel cyclic iminopyrimidines and their bicyclic compounds described therein, and their isotopic derivatives as well as pharmaceutical compositions containing such compounds. In addition, the present disclosure relates to the methods of preparing such compounds.

A kinase is an enzyme that catalyzes the transfer of phosphate groups from high-energy, phosphate-donating molecules to specific substrates. This process is known as phosphorylation, where the substrate gains a phosphate group and the high-energy ATP molecule donates a phosphate group. This transesterification produces a phosphorylated substrate and ADP.

Kinases are classified into broad groups by the substrate they act upon: protein kinases, lipid kinases, carbohydrate kinases. Kinases can be found in a variety of species, from bacteria to mold to worms to mammals. More than five hundred different kinases have been identified in humans.

MAP kinases (MAPKs) are a family of serine/threonine kinases that respond to a variety of extracellular growth signals. For example, growth hormone, epidermal growth factor, platelet-derived growth factor, and insulin are all considered mitogenic stimuli that can engage the MAPK pathway. Activation of this pathway at the level of the receptor initiates a signaling cascade whereby the Ras GTPase exchanges GDP for GTP. Next, Ras activates Raf kinase (also known as MAPKKK), which activates MEK (MAPKK). MEK activates MAPK (also known as ERK), which can go on to regulate transcription and translation. Whereas RAF and MAPK are both serine/threonine kinases, MAPKK is a tyrosine/threonine kinase.

The carcinogenic potential of the MAPK pathway makes it clinically significant. It is implicated in cell processes that can lead to uncontrolled growth and subsequent tumor formation. Mutations within this pathway alter its regulatory effects on cell differentiation, proliferation, survival, and apoptosis, all of which are implicated in various forms of cancer.

It is known that such kinases are frequently aberrantly expressed in common human cancers such as melanoma, colorectal cancer, thyroid cancer, glioma, breast cancer and lung cancer. It has also been shown that B-Raf, which possesses kinase activity, is mutated and/or overactive in many human cancers such as brain, lung, melanoma, colorectal cancer, ovarian cancer and papillary thyroid cancer.

Inhibition of the kinase is a useful method for disrupting the growth of mammalian cancer cells, therefore, for treating certain forms of cancer. Various compounds, such as pyrrolopyridine and anilinopyrimidine derivatives, have been shown to possess kinase inhibitory properties. Many patent publications refer to certain bicyclic derivatives, in particular quinazolinone derivatives.

Several compounds with diversified chemical structures have been developed into EphA2, Src and PAKs inhibitors, and two of them (FRAX486 and G-5555) are described as potent PAK1 inhibitors. For example, FRAX486 inhibits the PAK1 enzyme at a low nanomolar range.

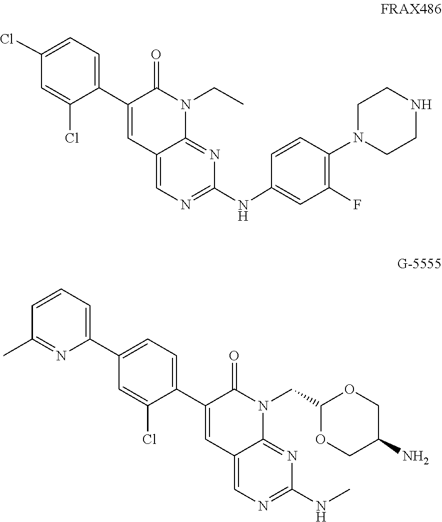

However, due to their structural characteristics, many of these kinase inhibitors exhibit poor pharmacokinetical properties, and some of them are substrates of active transporters such as P-glycoproteins (P-gp) or breast cancer resistance protein (BCRP), and have very low tendency to penetrate into cell membrane, as well as into brain. Therefore, they are not suitable to be used for the treatment of tumors or cancers in the brain, which is protected by the blood-brain barrier (BBB).

Thus, the compounds of the present disclosure, which are selective inhibitors of certain kinases, are useful in the treatment of abnormal cell growth, in particular cancers in mammals. In addition, these compounds have good penetration of cell membrane, therefore, are useful for treating tumors or cancers, including brain tumors, in humans.

### SUMMARY

In one aspect, provided is a compound of Formula (I):

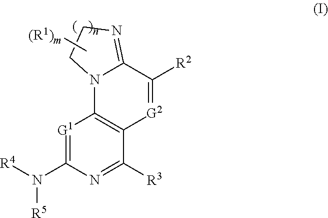

or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
   or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, optionally substituted aryl, optionally substituted aryl-O, optionally substituted aryl-S, optionally substituted aryl-SO₂, optionally substituted aryl-NR^{a}, optionally substituted heteroaryl, optionally substituted heteroaryl-O, optionally substituted heteroaryl-S, optionally substituted heteroaryl-SO₂, optionally substituted heteroaryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, optionally substituted C₁-C₆ alkyl, and optionally substituted C₁-C₆ alkoxy;
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
   (iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl; and
provided that when R² is 2,6-dichloro-3,5-dimethoxyphenyl, then:
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl.

Provided in other aspects are compounds of Formula (I-1a),

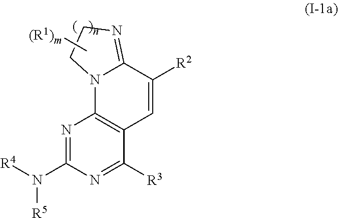

or a pharmaceutically acceptable salt, solvate or isotopic derivative thereof, wherein R¹, R², R³, R⁴, R⁵, m, and n are as defined for Formula (I).

Provided in other aspects are compounds of Formula (I-2a) or (I-2b):

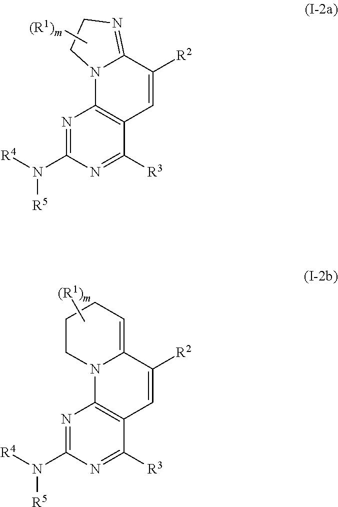

or a pharmaceutically acceptable salt, solvate or isotopic derivative thereof, wherein R¹, R², R³, R⁴, R⁵, and m are as defined for Formula (I).

Provided in other aspects are compounds of Formula (I-3a) or (I-3b):

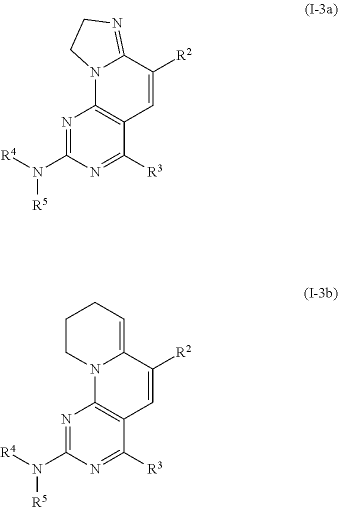

or a pharmaceutically acceptable salt, solvate or isotopic derivative thereof, wherein R², R³, R⁴, and R⁵ are as defined for Formula (I).

Provided in some embodiments are compounds of Table 1 or Table 2, or a pharmaceutically acceptable salt, solvate or isotopic derivative thereof.

In some aspects, provided are pharmaceutical compositions containing a compound of any of the formulae described herein, or a pharmaceutically acceptable salt, solvate or isotopic derivative thereof, and a pharmaceutically acceptable diluent or carrier.

In some aspects, provided are combinations containing at least one compound of Formula (I), (I-1a), (I-2a), (I-2b), (I-3a), (I-3b), or a compound of Table 1 or Table 2, or a pharmaceutically acceptable salt, solvate or isotopic derivative thereof, and a second prophylactic or therapeutic agent.

In some aspects, provided are compounds of Formula (I), such as compounds of Formula (I), (I-1a), (I-2a), (I-2b), (I-3a), (I-3b), or a compound of Table 1 or Table 2, or a pharmaceutically acceptable salt, solvate or isotopic derivative thereof, for use in treating and/or preventing a proliferation disorder, such as a cancer, or a tumor in a subject. In some embodiments, the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

Provided in some aspects are methods of treating and/or preventing a proliferation disorder, such as a cancer, or a tumor in a subject, wherein the method includes administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein. In some embodiments, the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

In some aspects, the present disclosure provides use of at least one compound of any of the formulae described herein, or a pharmaceutically acceptable salt, solvate or isotopic derivative thereof, for the manufacture of a medicament.

In some aspects, the present disclosure provides a method for producing an anti-proliferative or anti-metastatic effect in a subject having a proliferation disorder, a cancer, or a tumor which is sensitive to inhibition of relevant kinases, such as MAPK, PDGFR, Src, PAKs, c-Kit, EphA2, EphB4, FGFR, Axl, and c-Met, including administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein.

In some aspects, provided are compounds of Formula (I), such as compounds of Formula (I), (I-1a), (I-2a), (I-2b), (I-3a), (I-3b), or a compound of Table 1 or Table 2, or a pharmaceutically acceptable salt, solvate or isotopic derivative thereof, for use in the treatment of a neurodegenerative disease. In some embodiments, the neurodegenerative disease is selected from the group consisting of Amyotrophic lateral sclerosis, Parkinson's disease, Alzheimer's disease, and Huntington's disease.

In some aspects, the present disclosure provides a method for treating a neurodegenerative disease in a subject. In some embodiments, the method includes administering to the subject an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein. In some embodiments, the neurodegenerative disease is selected from the group consist of Amyotrophic lateral sclerosis, Parkinson's disease, Alzheimer's disease, and Huntington's disease.

In yet another aspect, provided are methods for inhibiting an activity of one or more kinases, such as MAPK, PDGFR, Src, PAKs, c-Kit, EphA2, EphB4, FGFR, Axl, and c-Met, in a cell, including contacting the cell with an effective amount of a compound of any of the formulae presented herein, or a pharmaceutically acceptable salt, solvate or isotopic derivative thereof, or a pharmaceutical composition containing a compound of any of the formulae disclosed herein, or a combination containing any of the formulae disclosed herein, wherein the contacting is in vitro, ex vivo, or in vivo.

### DETAILED DESCRIPTION

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

As used herein, reference to “about” a value or parameter herein includes (and describes) embodiments that are directed to that value or parameter per se. For example, description referring to “about X” includes description of “X”.

Unless clearly indicated otherwise, “an individual” or “a subject” as used herein intends a mammal, including but not limited to a human, bovine, primate, equine, canine, feline, porcine, and ovine animals. Thus, the compositions and methods provided herein have use in both human medicine and in the veterinary context, including use in agricultural animals and domestic pets. The individual may be a human who has been diagnosed with or is suspected of having a condition described herein, such as cancer. The individual may be a human who exhibits one or more symptoms associated with a condition described herein, such as cancer. The individual may be a human who has a mutated or abnormal gene associated with a condition described herein, such as cancer. The individual may be a human who is genetically or otherwise predisposed to or at risk of developing a condition described herein, such as cancer.

As used herein, “treatment” or “treating” is an approach for obtaining beneficial or desired results including clinical results. For purposes of the compositions and methods provided herein, beneficial or desired clinical results include, but are not limited to, one or more of the following: decreasing one or more symptoms resulting from the condition, diminishing the extent of the condition, stabilizing the condition (e.g., preventing or delaying the worsening of the condition), preventing or delaying the spread (e.g., metastasis) of the condition, delaying or slowing the progression of the condition, ameliorating a disease state, providing a remission (whether partial or total) of a disease, decreasing the dose of one or more other medications required to treat the condition, enhancing the effect of another medication used to treat the condition, increasing the quality of life of an individual having the condition, and/or prolonging survival. A method of treating cancer encompasses a reduction of the pathological consequence of cancer. The methods described herein contemplate any one or more of these aspects of treatment.

As used herein, an “at risk” individual is an individual who is at risk of developing a disease or condition described herein, such as cancer. An individual “at risk” may or may not have detectable disease, and may or may not have displayed detectable disease prior to the treatment methods described herein. “At risk” denotes that an individual has one or more so-called risk factors, which are measurable parameters that correlate with development of a disease or condition described herein, such as cancer. An individual having one or more of these risk factors has a higher probability of developing the disease or condition than an individual without these risk factor(s).

As used herein, by “combination therapy” is meant a therapy that includes two or more different compounds. Thus, in one aspect, a combination therapy comprising a compound detailed herein and another compound is provided. In some variations, the combination therapy optionally includes one or more pharmaceutically acceptable carriers or excipients, non-pharmaceutically active compounds, and/or inert substances. In various embodiments, treatment with a combination therapy may result in an additive or even synergistic (e.g., greater than additive) result compared to administration of a single compound provided herein alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency, and/or duration of one or more side-effects associated with the compound.

As used herein, the term “effective amount” intends such amount of a compound provided herein which in combination with its parameters of efficacy and toxicity, should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds. In various embodiments, an effective amount of the composition or therapy may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of a disease or condition described herein, such as cancer.

As is understood in the art, an “effective amount” may be in one or more doses, i.e., a single dose or multiple doses may be required to achieve the desired treatment endpoint. An effective amount may be considered in the context of administering one or more therapeutic agents, and a compound, or pharmaceutically acceptable salt thereof, may be considered to be given in an effective amount if, in conjunction with one or more other agents, a desirable or beneficial result may be or is achieved.

A “therapeutically effective amount” refers to an amount of a compound or salt thereof sufficient to produce a desired therapeutic outcome (e.g., reducing the severity or duration of, stabilizing the severity of, or eliminating one or more symptoms of a disease or condition described herein, such as cancer). For therapeutic use, beneficial or desired results include, e.g., decreasing one or more symptoms resulting from the disease (biochemical, histologic and/or behavioral), including its complications and intermediate pathological phenotypes presenting during development of the disease or condition, increasing the quality of life of those suffering from the disease or condition, decreasing the dose of other medications required to treat the disease or condition, enhancing effect of another medication, delaying the progression of the disease or condition, and/or prolonging survival of patients.

It is understood that an effective amount of a compound or pharmaceutically acceptable salt thereof, including a prophylactically effective amount, may be given to an individual in the adjuvant setting, which refers to a clinical setting in which an individual has had a history of cancer, and generally (but not necessarily) has been responsive to therapy, which includes, but is not limited to, surgery (e.g., surgical resection), radiotherapy, and chemotherapy. However, because of their history of cancer, these individuals are considered at risk of developing cancer. Treatment or administration in the “adjuvant setting” refers to a subsequent mode of treatment.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound provided herein in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound provided herein as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Alkyl” refers to and includes saturated linear or branched univalent hydrocarbon structures and combinations thereof. Particular alkyl groups are those having 1 to 20 carbon atoms (a “C₁-C₂₀ alkyl”). More particular alkyl groups are those having 1 to 8 carbon atoms (a “C₁-C₈ alkyl”) or 1 to 6 carbon atoms (a “C₁-C₆ alkyl”). When an alkyl residue having a specific number of carbons is named, all geometric isomers having that number of carbons are intended to be encompassed and described; thus, for example, “butyl” is meant to include n-butyl, sec-butyl, iso-butyl, and tert-butyl; “propyl” includes n-propyl and iso-propyl. This term is exemplified by groups such as methyl, t-butyl, n-heptyl, octyl, and the like.

“Cycloalkyl” refers to and includes cyclic univalent hydrocarbon structures. Cycloalkyl can consist of one ring, such as cyclohexyl, or multiple rings, such as adamantyl. A cycloalkyl comprising more than one ring may be fused, spiro or bridged, or combinations thereof. A preferred cycloalkyl is a saturated cyclic hydrocarbon having from 3 to 13 annular carbon atoms. A more preferred cycloalkyl is a saturated cyclic hydrocarbon having from 3 to 8 annular carbon atoms (a “C₃-C₈ cycloalkyl”). Examples of cycloalkyl groups include adamantyl, decahydronaphthalenyl, cyclopropyl, cyclobutyl, cyclopentyl, cyclohexyl, and the like.

“Alkenyl” refers to an unsaturated hydrocarbon group having at least one site of olefinic unsaturation (i.e., having at least one moiety of the formula C═C) and preferably having from 2 to 10 carbon atoms and more preferably 2 to 8 carbon atoms. Examples of alkenyl include but are not limited to —CH₂—CH═CH—CH₃ and —CH═CH—CH═CH₂.

“Cycloalkenyl” refers to an unsaturated hydrocarbon group within a cycloalkyl having at least one site of olefinic unsaturation (i.e., having at least one moiety of the formula C═C). Cycloalkenyl can consist of one ring, such as cyclohexyl, or multiple rings, such as norbornenyl. A more preferred cycloalkenyl is an unsaturated cyclic hydrocarbon having from 3 to 8 annular carbon atoms (a “C₃-C₈ cycloalkenyl”). Examples of cycloalkenyl groups include cyclopropenyl, cyclobutenyl, cyclopentenyl, cyclohexenyl, and the like.

“Alkynyl” refers to an unsaturated hydrocarbon group having at least one site of acetylenic unsaturation (i.e., having at least one moiety of the formula C≡C) and preferably having from 2 to 10 carbon atoms and more preferably 2 to 8 carbon atoms and the like.

The term “alkoxy” refers to an —O-alkyl group, where the O is the point of attachment to the rest of the molecule, and alkyl is as defined above.

The term “thioalkoxy” refers to an —S-alkyl group, where the S is the point of attachment to the rest of the molecule, and alkyl is as defined above.

“Haloalkyl” refers to an alkyl group with one or more halo substituents, such as one, two, or three halo substituents. Examples of haloalkyl groups include —CF₃, —(CH₂)F, —CHF₂, CH₂Br, —CH₂CF₃, —CH₂CHF₂, and —CH₂CH₂F.

“Carbocycle”, “carbocyclic”, or “carbocyclyl” refers to a saturated or an unsaturated non-aromatic cyclic hydrocarbon group having a single ring or multiple condensed rings having from 3 to 13 annular carbon atoms. A carbocycle comprising more than one ring may be fused, spiro or bridged, or any combination thereof. In fused ring systems, one or more of the rings can be aryl. A carbocycle having more than one ring where at least one ring is aromatic may be connected to the parent structure at either a non-aromatic ring position or at an aromatic ring position. In one variation, a carbocycle having more than one ring where at least one ring is aromatic is connected to the parent structure at a non-aromatic ring position.

“Heterocycle”, “heterocyclic”, or “heterocyclyl” refers to a saturated or an unsaturated non-aromatic group having a single ring or multiple condensed rings, and having from 1 to 10 annular carbon atoms and from 1 to 4 annular heteroatoms, such as nitrogen, sulfur or oxygen, and the like. A heterocycle comprising more than one ring may be fused, spiro or bridged, or any combination thereof. In fused ring systems, one or more of the rings can be aryl or heteroaryl. A heterocycle having more than one ring where at least one ring is aromatic may be connected to the parent structure at either a non-aromatic ring position or at an aromatic ring position. In one variation, a heterocycle having more than one ring where at least one ring is aromatic is connected to the parent structure at a non-aromatic ring position.

“Aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” or “HetAr” refers to an unsaturated aromatic carbocyclic group having from 1 to 10 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

The term “halogen” represents chlorine, fluorine, bromine, or iodine. The term “halo” represents chloro, fluoro, bromo, or iodo.

The term “substituted” means that the specified group or moiety bears one or more substituents including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

A composition of “substantially pure” compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

Any formula given herein is intended to represent compounds having structures depicted by the structural formula as well as certain variations or forms. In particular, compounds of any formula given herein, such compound of Formula (I), (I-1a), (I-2a), (I-2b), (I-3a), or (I-3b), may have asymmetric centers and therefore exist in different enantiomeric forms. These steromeric mixtures can be separated into their individual stereomers on the basis of their physical chemical or optical differences by methods known to those skilled in the art, for example, by chromatography or fractional crystallization. All such isomers, including diastereomers and enantiomers are considered as part of the invention. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Substitution with heavier isotopes such as deuterium (i.e., ²H) may afford certain therapeutic advantages resulting from greater metabolic stability, for example increased in vivo half-life or reduced dosage requirements. Isotopically labeled compounds described herein and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

### Compounds

Compounds and salts thereof (such as pharmaceutically acceptable salts) are detailed herein, including in the Summary and in the appended claims. Also provided are the use of all of the compounds described herein, including any and all stereoisomers, including geometric isomers (cis/trans), E/Z isomers, enantiomers, diastereomers, and mixtures thereof in any ratio including racemic mixtures, salts and solvates of the compounds described herein, as well as methods of making such compounds. Any compound described herein may also be referred to as a drug.

In one aspect, provided are compounds of Formula (I):

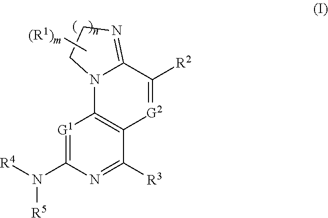

or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
   or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, optionally substituted aryl, optionally substituted aryl-O, optionally substituted aryl-S, optionally substituted aryl-SO₂, optionally substituted aryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heteroaryl, optionally substituted heteroaryl-O, optionally substituted heteroaryl-S, optionally substituted heteroaryl-SO₂, optionally substituted heteroaryl-NR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, optionally substituted C₁-C₆ alkyl, and optionally substituted C₁-C₆ alkoxy;
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
   (iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl; and
provided that when R² is 2,6-dichloro-3,5-dimethoxyphenyl, then:
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl.

In some embodiments, provided are compounds of Formula (I):

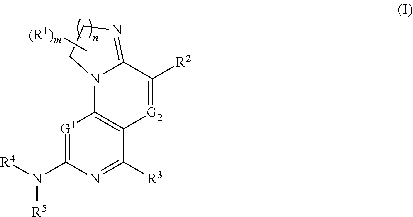

or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
   or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, optionally substituted aryl, optionally substituted aryl-O, optionally substituted aryl-S, optionally substituted aryl-SO₂, optionally substituted aryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heteroaryl, optionally substituted heteroaryl-0, optionally substituted heteroaryl-S, optionally substituted heteroaryl-SO₂, optionally substituted heteroaryl-NR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, optionally substituted C₁-C₆ alkyl, and optionally substituted C₁-C₆ alkoxy;
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl; and provided that when R² is 2,6-dichloro-3,5-dimethoxyphenyl, then:
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl.

In some embodiments, provided are compounds of Formula (I):

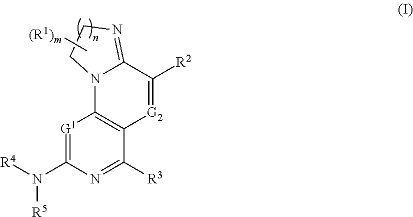

or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
   or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, aryl optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), aryl-O optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), optionally substituted aryl-S, optionally substituted aryl-SO₂, optionally substituted aryl-NR^{a}, heteroaryl optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), heteroaryl-O optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), optionally substituted heteroaryl-S, optionally substituted heteroaryl-SO₂, optionally substituted heteroaryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, optionally substituted C₁-C₆ alkyl, and optionally substituted C₁-C₆ alkoxy;
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
   (iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl; and
provided that when R² is 2,6-dichloro-3,5-dimethoxyphenyl, then:
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl.

In some embodiments, provided are compounds of Formula (I):

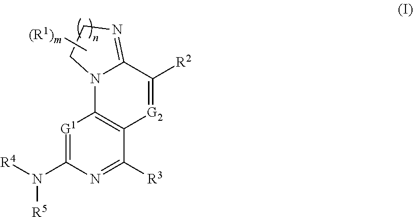

or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
   or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, aryl optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), aryl-O optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), optionally substituted aryl-S, optionally substituted aryl-SO₂, optionally substituted aryl-NR^{a}, heteroaryl optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), heteroaryl-O optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), optionally substituted heteroaryl-S, optionally substituted heteroaryl-SO₂, optionally substituted heteroaryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, optionally substituted C₁-C₆ alkyl, and optionally substituted C₁-C₆ alkoxy;
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl; and
provided that when R² is 2,6-dichloro-3,5-dimethoxyphenyl, then:
R⁴ is selected from the group consisting of:
   (i) hydrogen;
   (ii) C₁-C₆ alkyl optionally substituted with one or more groups selected from the group consisting of halogen, C₁-C₆ alkoxy, hydroxyl, heteroaryl, and optionally substituted heterocyclyl;
   (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, —C(O)O(C₁-C₆ alkyl), optionally substituted C₁-C₆ alkyl, optionally substituted C₁-C₆ alkoxy, optionally substituted C₁-C₆ thioalkoxy, and optionally substituted heterocyclyl;
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
   (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
   (vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl.

In some embodiments, provided is a compound of Formula (I):

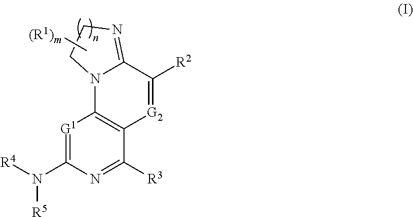

or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
   or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, optionally substituted aryl, optionally substituted aryl-O, optionally substituted aryl-S, optionally substituted aryl-SO₂, optionally substituted aryl-NR^{a}, optionally substituted heteroaryl, optionally substituted heteroaryl-O, optionally substituted heteroaryl-S, optionally substituted heteroaryl-SO₂, optionally substituted heteroaryl-NR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, cyano, nitro, optionally substituted C₁-C₆ alkyl, and optionally substituted C₁-C₆ alkoxy;
R⁴ is selected from the group consisting of:
   (i) hydrogen;
   (ii) C₁-C₆ alkyl optionally substituted with optionally substituted heterocyclyl; (iii) heterocyclyl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl;
   (iv) aryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl; and
   (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl; and
R⁵ is hydrogen or C₁-C₆ alkyl;
or R⁴ and R⁵ are taken together with the nitrogen to which they are attached to form an optionally substituted heterocyclyl.

In some embodiments of Formula (I), G¹ is N. In some embodiments, G¹ is CR^{a}, wherein R^{a} is selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy. In some embodiments, G¹ is CR^{a}, wherein R^{a} is selected from the group consisting of hydrogen, halogen, optionally substituted C₁-C₆ alkyl, and optionally substituted C₁-C₆ alkoxy. In some embodiments, G¹ is CH.

In some embodiments of Formula (I), G² is N. In some embodiments, G² is CR^{b}, wherein R^{b} is selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy. In some embodiments, G² is CR^{b}, wherein R^{b} is selected from the group consisting of hydrogen, halogen, optionally substituted C₁-C₆ alkyl, and optionally substituted C₁-C₆ alkoxy. In some embodiments, G² is CH. In some embodiments, G² is CR^{b}, wherein R^{b} is optionally substituted C₁-C₆ alkyl. In some embodiments, G² is CR^{b}, wherein R^{b} is C₁-C₆ alkyl. In some embodiments, G² is CR^{b}, wherein R^{b} is methyl.

In some embodiments of Formula (I), G¹ is N and G² is N. In some embodiments, G¹ is CR^{a} and G² is N. In certain embodiments, G¹ is CH and G² is N. In some embodiments, G¹ is N and G² is CR^{b}. In certain embodiments, G¹ is N and G² is CH. In some embodiments, G¹ is N and G² is CR^{b}, wherein R^{b} is C₁-C₆ alkyl. In some embodiments, G¹ is N G² is CR^{b}, wherein R^{b} is methyl. In other embodiments, G¹ is CR^{a} and G² is CR^{b}. In some embodiments, G¹ is CH and G² is CH.

In some embodiments, the compound of Formula (I) is a compound of Formula (I-1a),

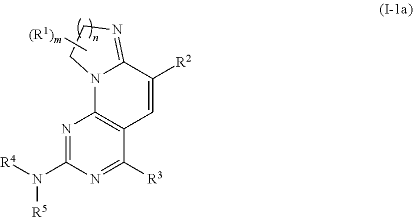

or a pharmaceutically acceptable salt, solvate or isotopic derivative thereof, wherein R¹, R², R³, R⁴, R⁵, m, and n are as defined for Formula (I).

In some embodiments of Formula (I) or (I-1a), n is 1. In other embodiments, n is 2.

In some embodiments, the compound of Formula (I) is a compound of Formula (I-2a) or (I-2b):

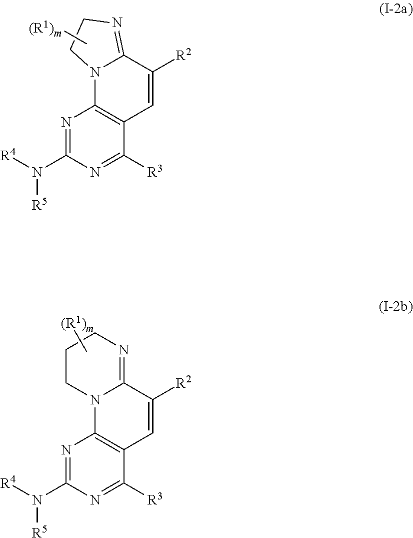

or a pharmaceutically acceptable salt, solvate or isotopic derivative thereof, wherein R¹, R², R³, R⁴, R⁵, and m are as defined for Formula (I).

In some embodiments of Formula (I), (I-1a), (I-2a), or (I-2b), m is 0. In some embodiments, m is 1. In some embodiments, m is 2. In other embodiments, m is 3. In some embodiments, m is 1, 2, or 3. In some embodiments, m is 1 and R¹ is independently selected from the group consisting of halogen, optionally substituted C₁-C₆ alkyl, and optionally substituted C₁-C₆ alkoxy. In some embodiments, m is 1 and R¹ is optionally substituted C₁-C₆ alkyl. In some embodiments, m is 1 and R¹ is C₁-C₆ alkyl. In some embodiments, m is 1 and R¹ is methyl. In some embodiments, m is 2 or 3, and each R¹ is independently selected from the group consisting of halogen, optionally substituted C₁-C₆ alkyl, and optionally substituted C₁-C₆ alkoxy; or two R¹ groups with the carbon atom they connect to form a 4- to 7-membered carbocyclic or heterocyclic ring, which is optionally substituted by one or more R^{a} groups. In some embodiments, m is 2 or 3, and each R¹ is independently selected from the group consisting of halogen, optionally substituted C₁-C₆ alkyl, and optionally substituted C₁-C₆ alkoxy. In some embodiments, m is 2 and each R¹ is independently selected from the group consisting of halogen, optionally substituted C₁-C₆ alkyl, and optionally substituted C₁-C₆ alkoxy; or two R¹ groups with the carbon atom they connect to form a 4- to 7-membered carbocyclic or heterocyclic ring, which is optionally substituted by one or more R^{a} groups. In some embodiments, m is 2 and the two R¹ groups with the carbon atom they connect to form a 4- to 7-membered carbocyclic or heterocyclic ring, which is optionally substituted by one or more R^{a} groups. In some embodiments, m is 2 and the two R¹ groups with the carbon atom they connect to form a cyclopentane ring.

In some embodiments, the compound of Formula (I) is a compound of Formula (I-3a) or (I-3b):

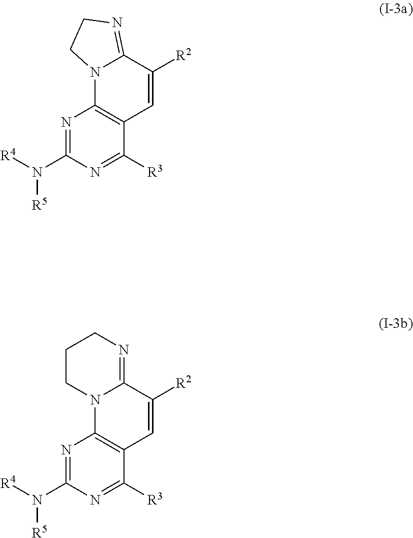

or a pharmaceutically acceptable salt, solvate or isotopic derivative thereof, wherein R², R³, R⁴, and R⁵ are as defined for Formula (I).

In some embodiments of Formula (I), (I-1a), (I-2a), (I-2b), (I-3a), or (I-3b), R³ is hydrogen. In some embodiments, R³ is selected from the group consisting of hydrogen, halogen, cyano, nitro, optionally substituted C₁-C₆ alkyl, and optionally substituted C₁-C₆ alkoxy. In some embodiments, R³ is selected from the group consisting of hydrogen, halogen, optionally substituted C₁-C₆ alkyl, and optionally substituted C₁-C₆ alkoxy. In some embodiments, R³ is selected from the group consisting of halogen, optionally substituted C₁-C₆ alkyl, and optionally substituted C₁-C₆ alkoxy. In some embodiments, R³ is optionally substituted C₁-C₆ alkyl. In some embodiments, R³ is C₁-C₆ alkyl. In some embodiments, R³ is methyl.

In some embodiments of Formula (I), (I-1a), (I-2a), (I-2b), (I-3a), or (I-3b), R² is selected from the group consisting of hydrogen, optionally substituted C₁-C₆ alkyl, optionally substituted C₁-C₆ alkyl-0, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, optionally substituted aryl, optionally substituted aryl-O, optionally substituted aryl-S, optionally substituted aryl-SO₂, optionally substituted aryl-NR^{a}, optionally substituted heteroaryl, optionally substituted heteroaryl-O, optionally substituted heteroaryl-S, optionally substituted heteroaryl-SO₂, optionally substituted heteroaryl-NR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a}. In some embodiments, R² is selected from the group consisting of hydrogen, optionally substituted C₁-C₆ alkyl, optionally substituted C₁-C₆ alkyl-0, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, aryl optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), aryl-O optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), optionally substituted aryl-S, optionally substituted aryl-SO₂, optionally substituted aryl-NR^{a}, heteroaryl optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), heteroaryl-O optionally substituted with one or more groups selected from the group consisting of halogen, hydroxyl, amino, cyano, optionally substituted C₁-C₆ alkyl, optionally substituted C₁-C₆ alkoxy, optionally substituted C₁-C₆ thioalkoxy, NR^{a}CO-(optionally substituted C₁-C₆ alkyl), NR^{a}CO-(optionally substituted aryl), NR^{a}CO-(optionally substituted heteroaryl), NR^{a}CO-(optionally substituted C₂-C₆ alkynyl), NR^{a}CO-(optionally substituted cycloalkyl), NR^{a}CO-(optionally heterocyclyl), CONR^{a}-(optionally substituted C₁-C₆ alkyl), CONR^{a}-(optionally substituted aryl), CONR^{a}-(optionally substituted heteroaryl), CONR^{a}-(optionally substituted C₂-C₆ alkynyl), CONR^{a}-(optionally substituted cycloalkyl), CONR^{a}-(optionally heterocyclyl), NR^{a}SO₂-(optionally substituted C₁-C₆ alkyl), NR^{a}SO₂-(optionally substituted aryl), NR^{a}SO₂-(optionally substituted heteroaryl), NR^{a}SO₂-(optionally substituted C₂-C₆ alkynyl), NR^{a}SO₂-(optionally substituted cycloalkyl), NR^{a}SO₂-(optionally heterocyclyl), SO₂NR^{a}-(optionally substituted C₁-C₆ alkyl), SO₂NR^{a}-(optionally substituted aryl), SO₂NR^{a}-(optionally substituted heteroaryl), SO₂NR^{a}-(optionally substituted C₂-C₆ alkynyl), SO₂NR^{a}-(optionally substituted cycloalkyl), SO₂NR^{a}-(optionally heterocyclyl), NR^{a}CONR^{a}-(optionally substituted C₁-C₆ alkyl), NR^{a}CONR^{a}-(optionally substituted aryl), NR^{a}CONR^{a}-(optionally substituted heteroaryl), NR^{a}CONR^{a}-(optionally substituted C₂-C₆ alkynyl), NR^{a}CONR^{a}-(optionally substituted cycloalkyl), NR^{a}CONR^{a}-(optionally heterocyclyl), NR^{a}CO-cycloalkylene-CONR^{a}-(optionally substituted C₁-C₆ alkyl), NR^{a}CO-cycloalkylene-CONR^{a}-(optionally substituted aryl), NR^{a}CO-cycloalkylene-CONR^{a}-(optionally substituted heteroaryl), NR^{a}CO-cycloalkylene-CONR^{a}-(optionally substituted C₂-C₆ alkynyl), NR^{a}CO-cycloalkylene-CONR^{a}-(optionally substituted cycloalkyl), and (optionally heterocyclyl), optionally substituted heteroaryl-S, optionally substituted heteroaryl-SO₂, optionally substituted heteroaryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a}. In some embodiments, R² is selected from the group consisting of hydrogen, optionally substituted C₂-C₆ alkynyl, optionally substituted aryl, and optionally substituted heteroaryl. In some embodiments, R² is hydrogen. In some embodiments, R² is optionally substituted C₂-C₆ alkynyl. In some embodiments, R² is C₂-C₆ alkynyl substituted with aryl, heteroaryl, or heterocyclyl. In some embodiments, R² is C₂-C₆ alkynyl substituted with C₆-C₁₄ aryl, 5- to 12-membered heteroaryl, or 3- to 12-membered heterocyclyl. In some embodiments, R² is C₂-C₆ alkynyl substituted with 5- to 6-membered heteroaryl.

In some embodiments of Formula (I), (I-1a), (I-2a), (I-2b), (I-3a), or (I-3b), R² is optionally substituted aryl. In some embodiments, R² is optionally substituted C₆-C₁₄ aryl. In certain embodiments, R² is phenyl or napthyl. In some embodiments, R² is aryl substituted with one or more substituents selected from the group consisting of C₁-C₆ alkyl, C₁-C₆ alkoxy, halo, hydroxyl, optionally substituted heteroaryl, and optionally substituted heterocyclyl. In some embodiments, R² is C₆-C₁₄ aryl substituted with one or more substituents selected from the group consisting of C₁-C₆ alkyl, C₁-C₆ alkoxy, halo, hydroxyl, optionally substituted 5- to 12-membered heteroaryl, and optionally substituted 3- to 12-membered heterocyclyl. In certain embodiments, R² is phenyl substituted with one or more substituents selected from the group consisting of C₁-C₆ alkyl, C₁-C₆ alkoxy, halo, hydroxyl, optionally substituted 5- to 12-membered heteroaryl, and optionally substituted 3- to 12-membered heterocyclyl. In some embodiments, R² is C₆-C₁₄ aryl substituted one or two halo groups. In certain embodiments, R² is phenyl substituted with one or two halo groups. In some embodiments, R² is phenyl substituted with chloro. In some embodiments, R² is phenyl substituted with two chloro groups. In some embodiments, R² is C₆-C₁₄ aryl substituted with a halo substituent and an additional substituent selected from the group consisting of C₁-C₆ alkyl, C₁-C₆ alkoxy, hydroxyl, optionally substituted 5- to 12-membered heteroaryl, and optionally substituted 3- to 12-membered heterocyclyl. In some embodiments, R² is phenyl substituted with halo and optionally substituted 5- to 6-membered heteroaryl. In other embodiments, R² is phenyl substituted with halo and optionally substituted 5- to 6-membered heterocyclyl. In certain embodiments, R² is phenyl substituted with chloro and a 5- to 6-membered heterocyclyl that is optionally substituted one or more groups selected from oxo and C₁-C₆ alkyl. In other embodiments, R² is phenyl substituted with chloro and a 5- to 6-membered heteroaryl that is optionally substituted one or more groups selected from oxo and C₁-C₆ alkyl. In some embodiments, R² is C₆-C₁₄ aryl substituted with C₁-C₆ alkyl. In certain embodiments, R² is phenyl substituted with C₁-C₆ alkyl, such as phenyl substituted with methyl. In some embodiments, R² is C₆-C₁₄ aryl substituted with C₁-C₆ alkoxy. In some embodiments, R² is phenyl substituted with C₁-C₆ alkoxy, such as phenyl substituted with methoxy. In some embodiments, R² is C₆-C₁₄ aryl substituted with hydroxyl. In some embodiments, R² is phenyl substituted with hydroxyl. In other embodiments, R² is C₆-C₁₄ aryl substituted with one or more groups selected from C₁-C₆ alkyl, C₁-C₆ alkoxy, and hydroxyl.

In some embodiments of Formula (I), (I-1a), (I-2a), (I-2b), (I-3a), or (I-3b), R² is optionally substituted heteroaryl. In some embodiments, R² is optionally substituted 5- to 12-membered heteroaryl. In some embodiments, R² is optionally substituted 5- to 6-membered heteroaryl. In some embodiments, R² is an unsubstituted 5- to 6-membered heteroaryl. In some embodiments, R² is 5- to 6-membered heteroaryl optionally substituted with one or more groups selected from the group consisting of C₁-C₆ alkyl, C₁-C₆ alkoxy, and hydroxyl. In some embodiments, R² is 5- to 6-membered heteroaryl optionally substituted with C₁-C₆ alkyl. In some embodiments, R² is optionally substituted heterocyclyl. In some embodiments, R² is optionally substituted 3- to 12-membered heterocyclyl. In some embodiments, R² is optionally substituted 5- to 6-membered heterocyclyl. In some embodiments, R² is 5- to 6-membered heterocyclyl optionally substituted with one or more groups selected from the group consisting of C₁-C₆ alkyl, C₁-C₆ alkoxy, hydroxyl, and oxo.

In some embodiments of Formula (I), (I-1a), (I-2a), (I-2b), (I-3a), or (I-3b), R² is selected from the group consisting of H,

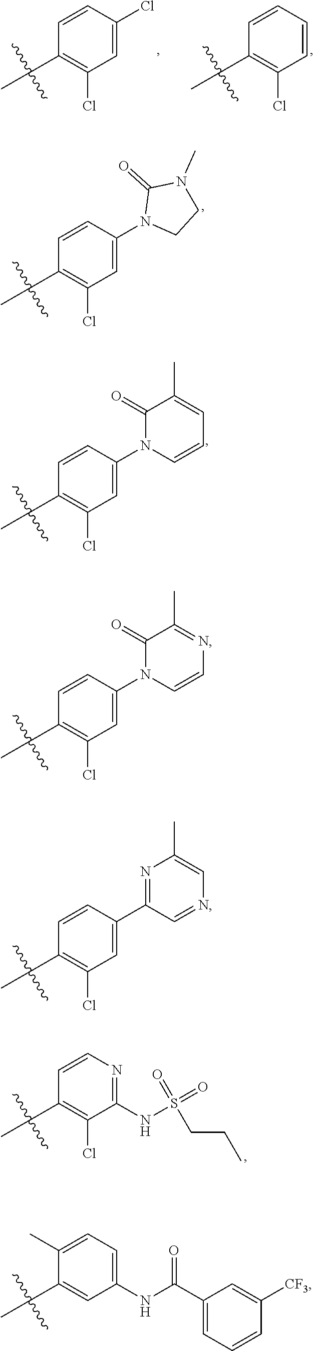

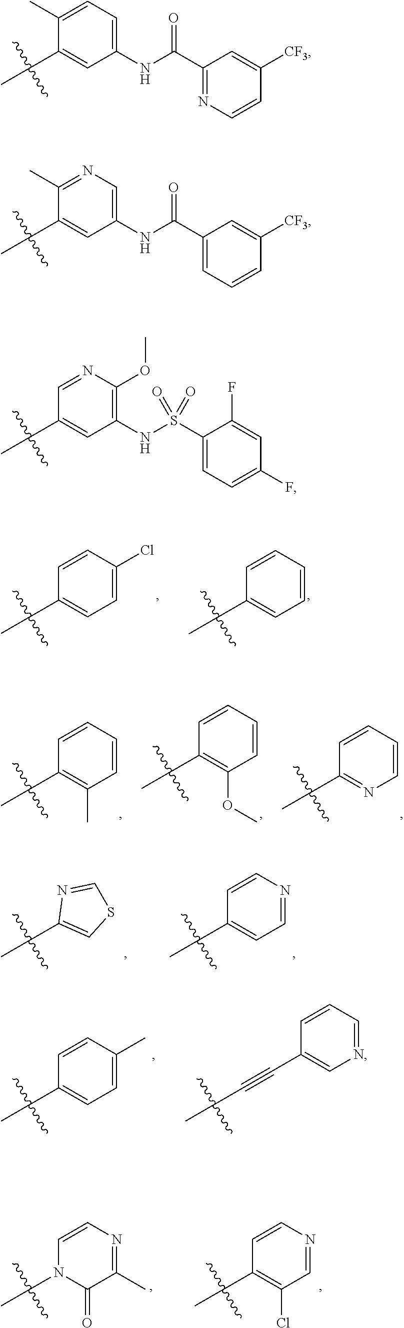

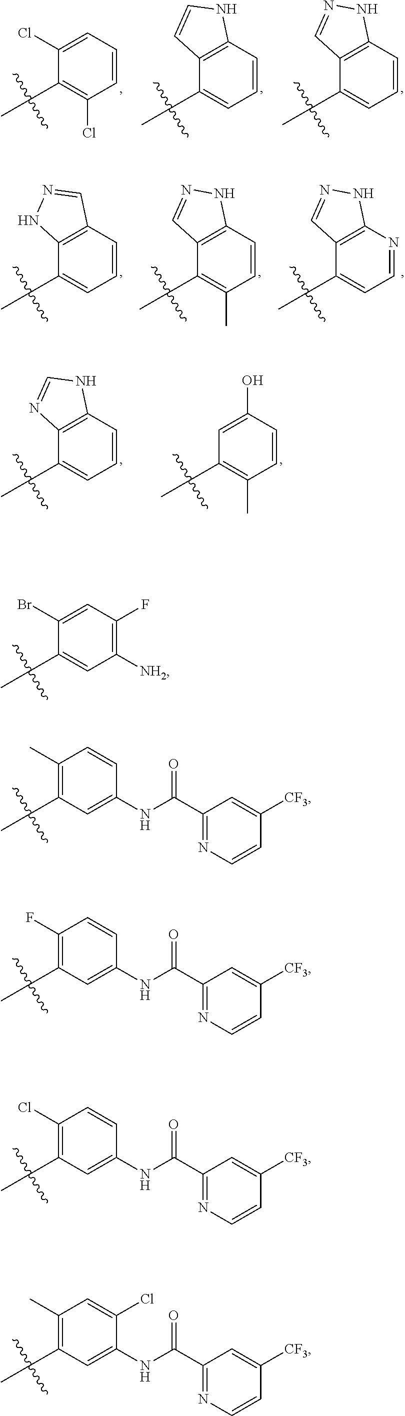

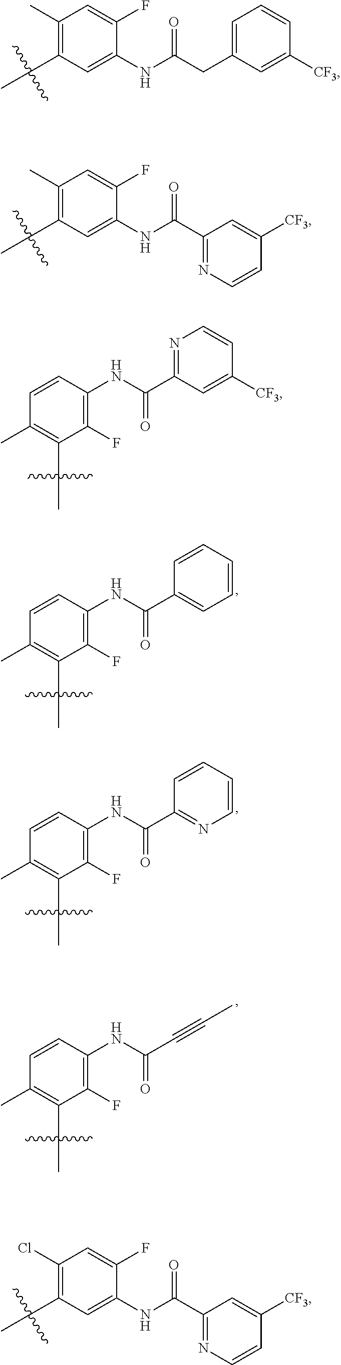

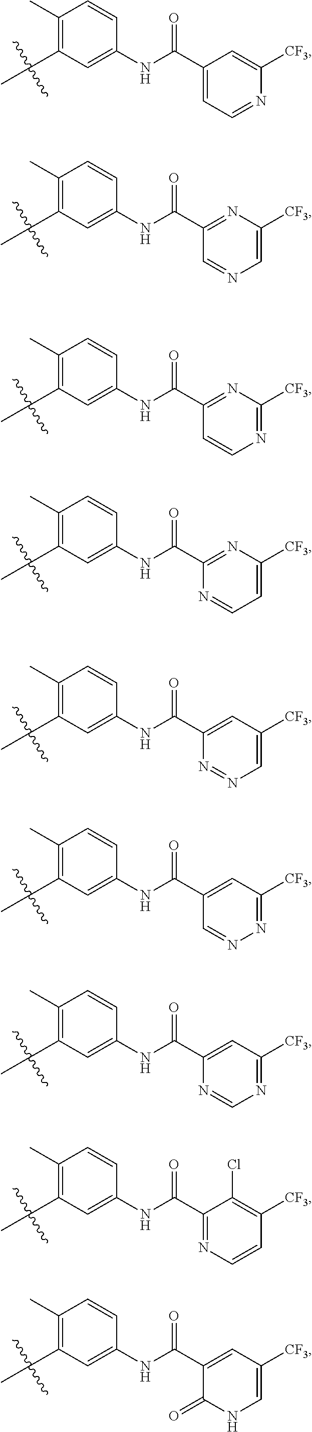

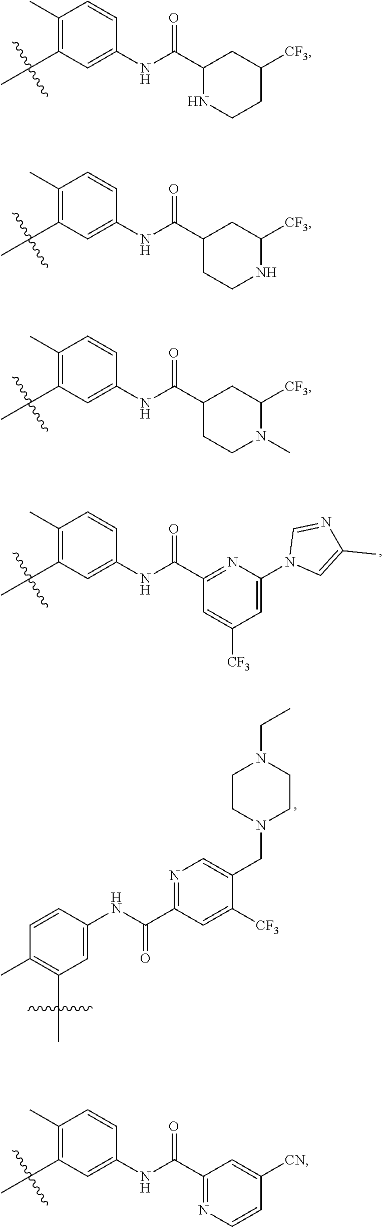

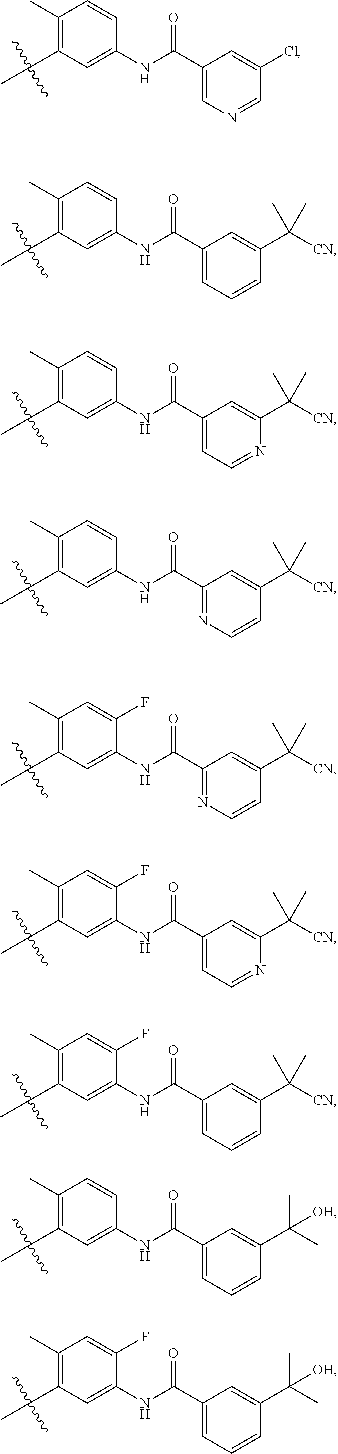

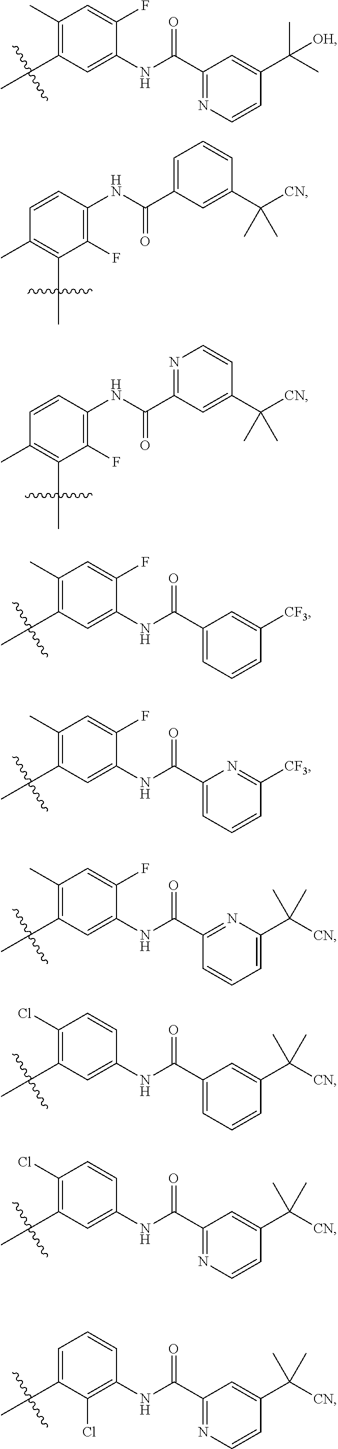

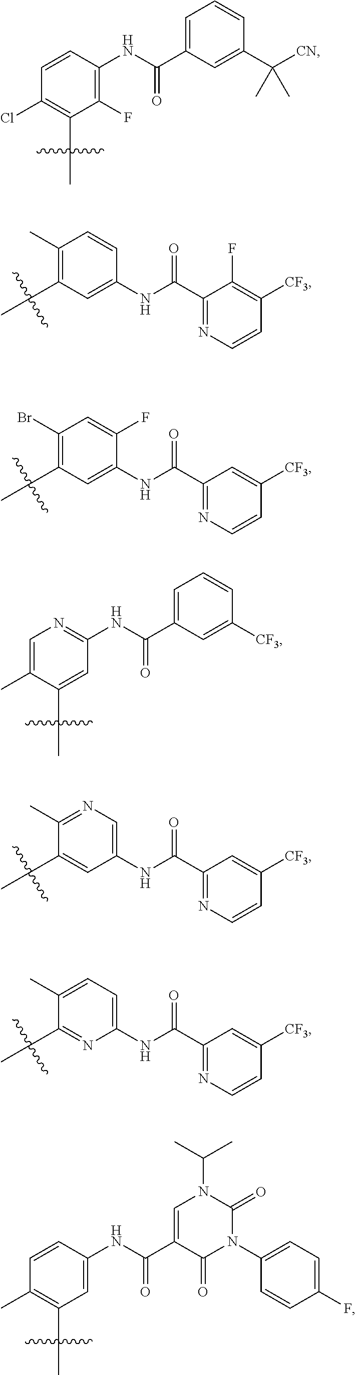

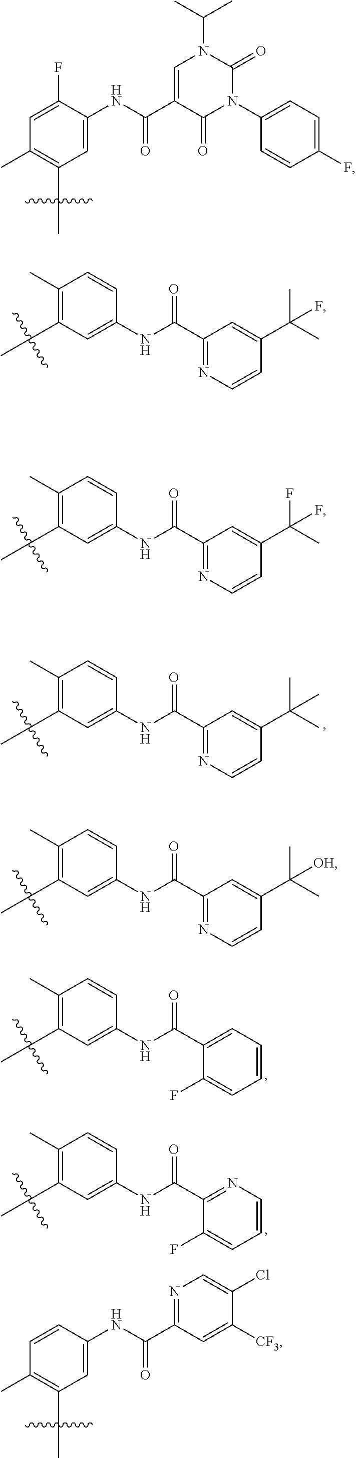

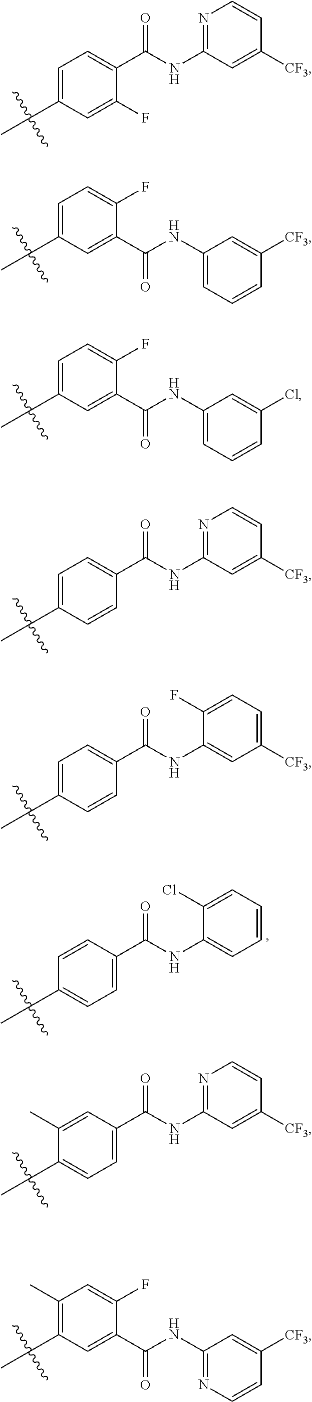

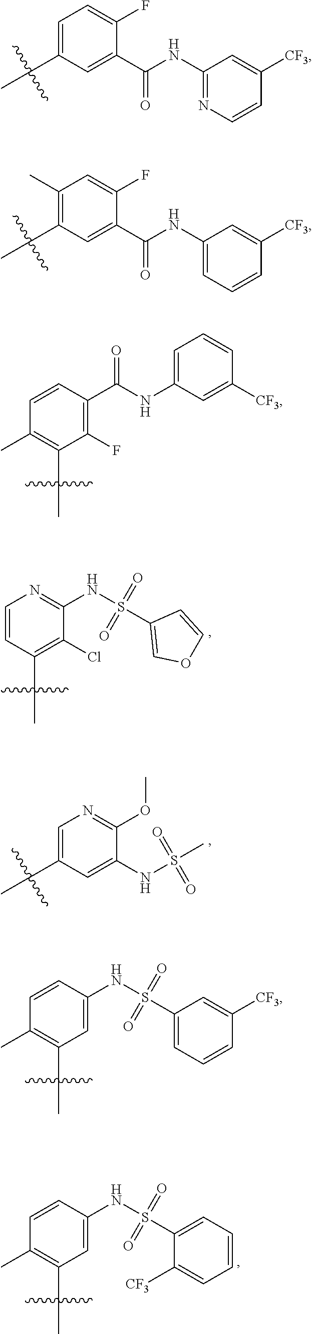

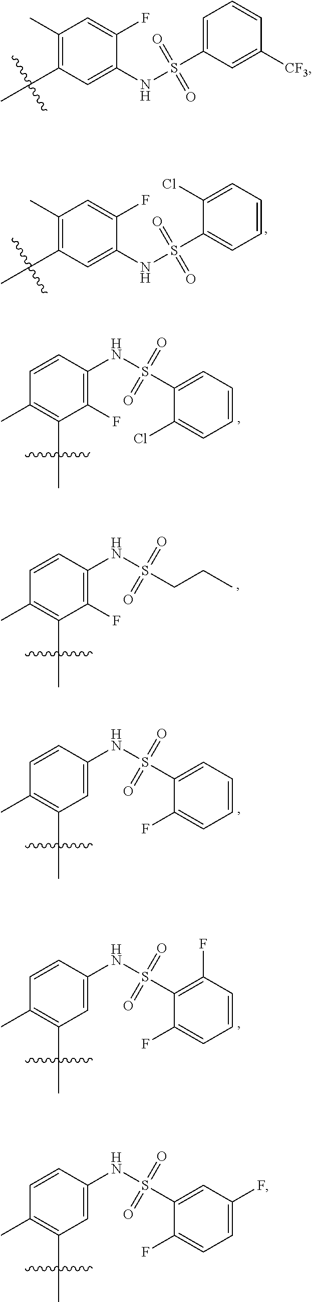

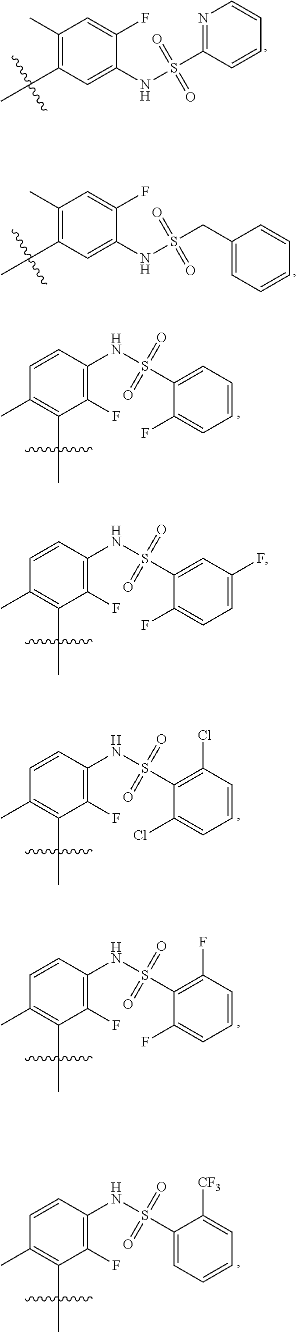

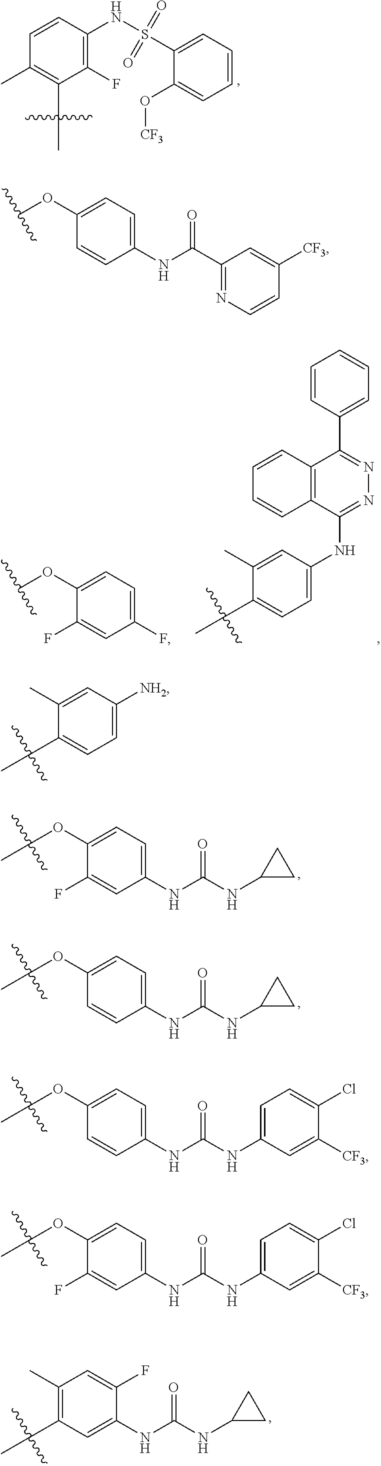

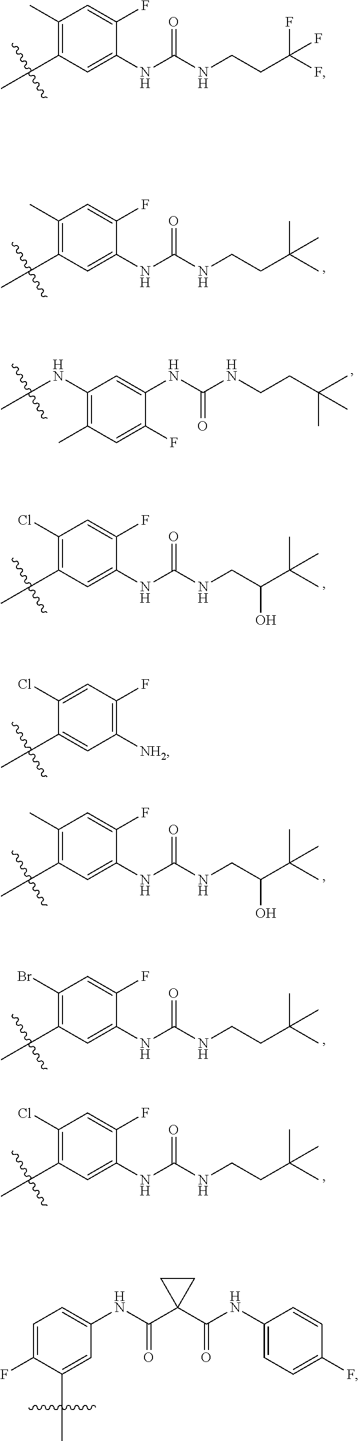

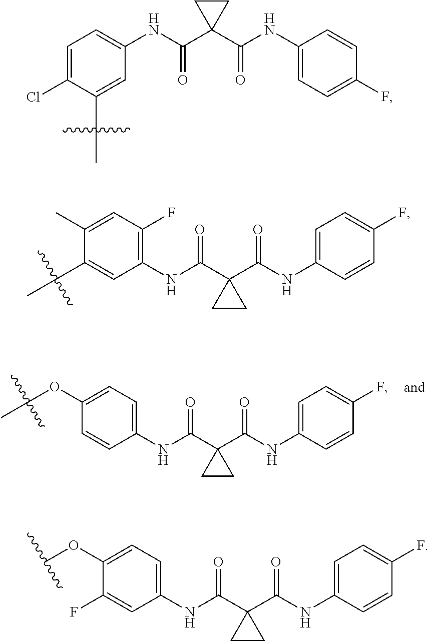 [SMILES: *c1cc(NC(=O)C2(C(=O)Nc3ccc(F)cc3)CC2)ccc1Cl ; *c1cc(NC(=O)C2(C(=O)Nc3ccc(F)cc3)CC2)c(F)cc1C ; *Oc1ccc(NC(=O)C2(C(=O)Nc3ccc(F)cc3)CC2)cc1 ; *Oc1ccc(NC(=O)C2(C(=O)Nc3ccc(F)cc3)CC2)cc1F]

In some embodiments, R² is selected from the group consisting of H,

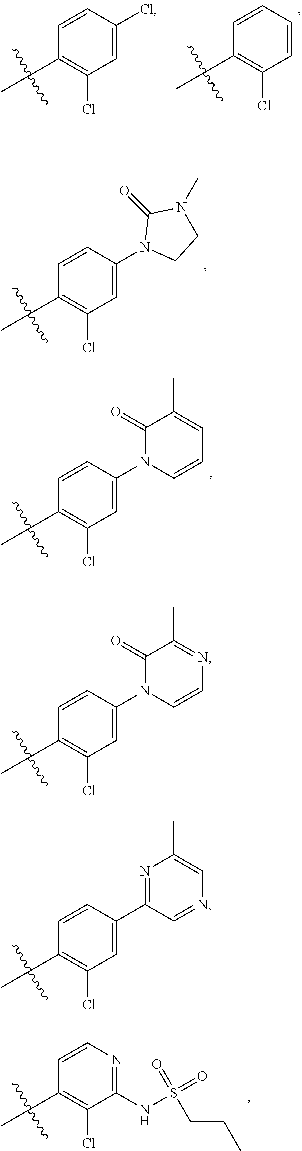

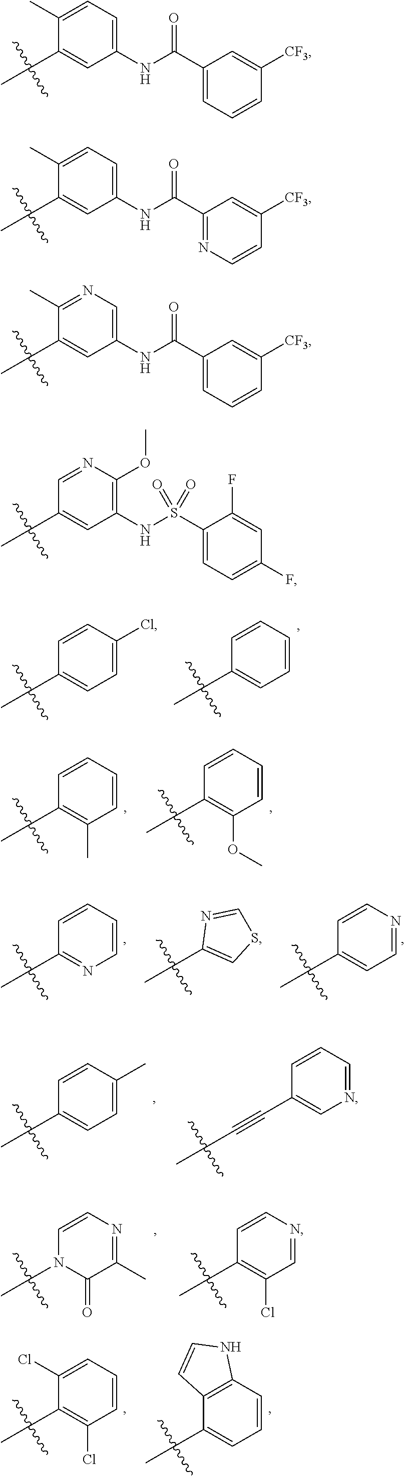

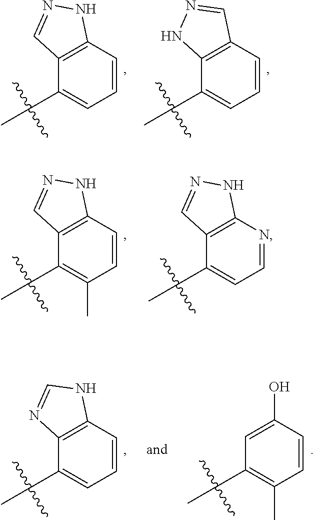

In some embodiments R² is H. In some embodiments R² is

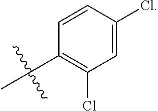 [SMILES: *c1ccc(Cl)cc1Cl]

In some embodiments R² is

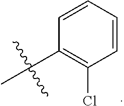 [SMILES: *c1ccccc1Cl]

In some embodiments R² is

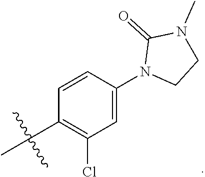 [SMILES: *c1ccc(N2CCN(C)C2=O)cc1Cl]

In some embodiments R² is

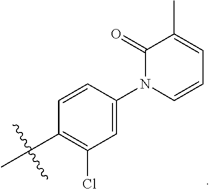 [SMILES: *c1ccc(-n2cccc(C)c2=O)cc1Cl]

In some embodiments R² is

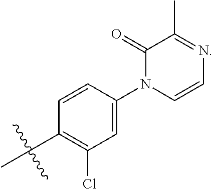 [SMILES: *c1ccc(-n2ccnc(C)c2=O)cc1Cl]

In some embodiments R² is

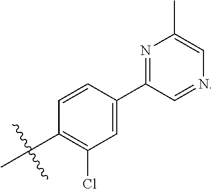 [SMILES: *c1ccc(-c2cncc(C)n2)cc1Cl]

In some embodiments R² is

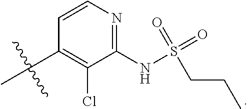 [SMILES: *c1ccnc(NS(=O)(=O)CCC)c1Cl]

In some embodiments R² is

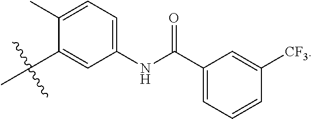 [SMILES: *c1cc(NC(=O)c2cccc(C(F)(F)F)c2)ccc1C]

In some embodiments R² is

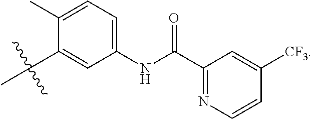 [SMILES: *c1cc(NC(=O)c2cc(C(F)(F)F)ccn2)ccc1C]

In some embodiments R² is

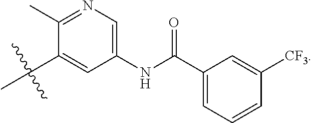 [SMILES: *c1cc(NC(=O)c2cccc(C(F)(F)F)c2)cnc1C]

In some embodiments R² is

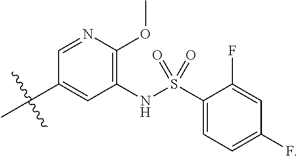 [SMILES: *c1cnc(OC)c(NS(=O)(=O)c2ccc(F)cc2F)c1]

In some embodiments R² is

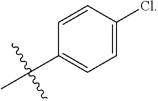 [SMILES: *c1ccc(Cl)cc1]

In some embodiments R² is

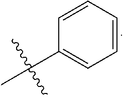 [SMILES: *c1ccccc1]

In some embodiments R² is

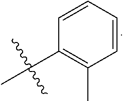 [SMILES: *c1ccccc1C]

In some embodiments R² is

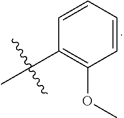 [SMILES: *c1ccccc1OC]

In some embodiments R² is

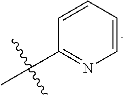 [SMILES: *c1ccccn1]

In some embodiments R² is

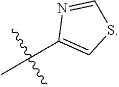 [SMILES: *c1cscn1]

In some embodiments R² is

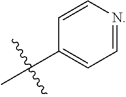 [SMILES: *c1ccncc1]

In some embodiments R² is

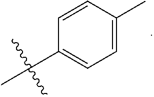 [SMILES: *c1ccc(C)cc1]

In some embodiments R² is

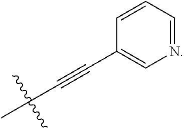 [SMILES: *C#Cc1cccnc1]

In some embodiments R² is

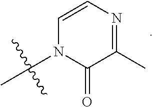 [SMILES: *n1ccnc(C)c1=O]

In some embodiments R² is

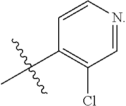 [SMILES: *c1ccncc1Cl]

In some embodiments R² is

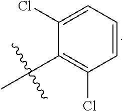 [SMILES: *c1c(Cl)cccc1Cl]

In some embodiments R² is

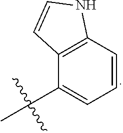 [SMILES: *c1cccc2[nH]ccc12]

In some embodiments R² is

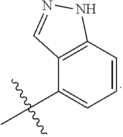 [SMILES: *c1cccc2[nH]ncc12]

In some embodiments R² is

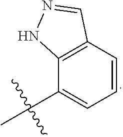 [SMILES: *c1cccc2cn[nH]c12]

In some embodiments R² is

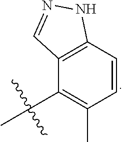 [SMILES: *c1c(C)ccc2[nH]ncc12]

In some embodiments R² is

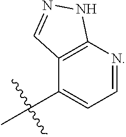 [SMILES: *c1ccnc2[nH]ncc12]

In some embodiments R² is

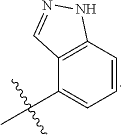 [SMILES: *c1cccc2[nH]ncc12]

In some embodiments R² is

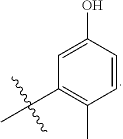 [SMILES: *c1cc(O)ccc1C]

In some embodiments R² is

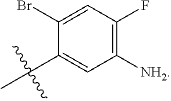 [SMILES: *c1cc(N)c(F)cc1Br]

In some embodiments R² is

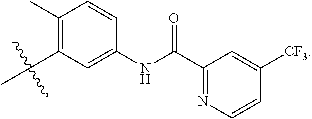 [SMILES: *c1cc(NC(=O)c2cc(C(F)(F)F)ccn2)ccc1C]

In some embodiments R² is

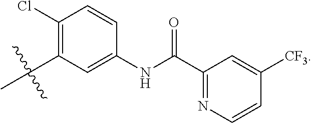 [SMILES: *c1cc(NC(=O)c2cc(C(F)(F)F)ccn2)ccc1Cl]

In some embodiments R² is

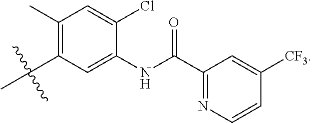 [SMILES: *c1cc(NC(=O)c2cc(C(F)(F)F)ccn2)c(Cl)cc1C]

In some embodiments R² is

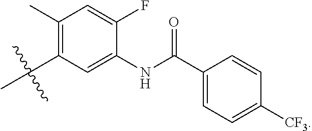 [SMILES: *c1cc(NC(=O)c2ccc(C(F)(F)F)cc2)c(F)cc1C]

In some embodiments R² is

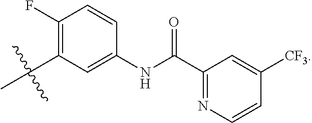 [SMILES: *c1cc(NC(=O)c2cc(C(F)(F)F)ccn2)ccc1F]

In some embodiments R² is

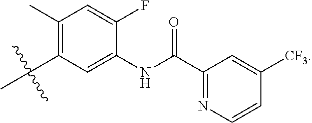 [SMILES: *c1cc(NC(=O)c2cc(C(F)(F)F)ccn2)c(F)cc1C]

In some embodiments R² is

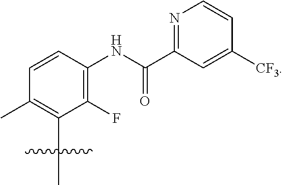 [SMILES: *c1c(C)ccc(NC(=O)c2cc(C(F)(F)F)ccn2)c1F]

In some embodiments R² is

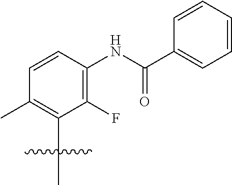 [SMILES: *c1c(C)ccc(NC(=O)c2ccccc2)c1F]

In some embodiments R² is

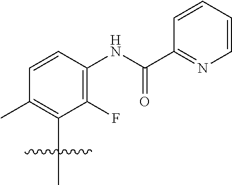 [SMILES: *c1c(C)ccc(NC(=O)c2ccccn2)c1F]

In some embodiments R² is

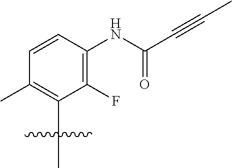 [SMILES: *c1c(C)ccc(NC(=O)C#CC)c1F]

In some embodiments R² is

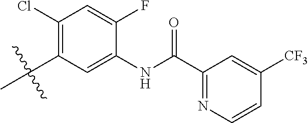 [SMILES: *c1cc(NC(=O)c2cc(C(F)(F)F)ccn2)c(F)cc1Cl]

In some embodiments R² is

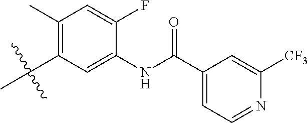 [SMILES: *c1cc(NC(=O)c2ccnc(C(F)(F)F)c2)c(F)cc1C]

In some embodiments R² is

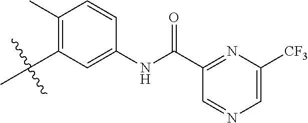 [SMILES: *c1cc(NC(=O)c2cncc(C(F)(F)F)n2)ccc1C]

In some embodiments R² is

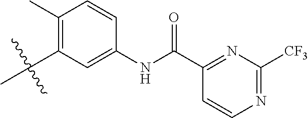 [SMILES: *c1cc(NC(=O)c2ccnc(C(F)(F)F)n2)ccc1C]

In some embodiments R² is

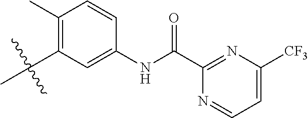 [SMILES: *c1cc(NC(=O)c2nccc(C(F)(F)F)n2)ccc1C]

In some embodiments R² is

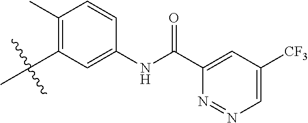 [SMILES: *c1cc(NC(=O)c2cc(C(F)(F)F)cnn2)ccc1C]

In some embodiments R² is

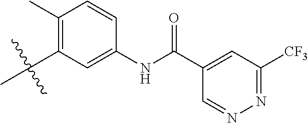 [SMILES: *c1cc(NC(=O)c2cnnc(C(F)(F)F)c2)ccc1C]

In some embodiments R² is

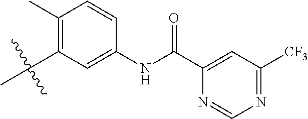 [SMILES: *c1cc(NC(=O)c2cc(C(F)(F)F)ncn2)ccc1C]

In some embodiments R² is

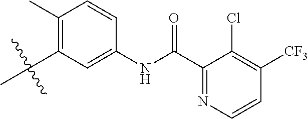 [SMILES: *c1cc(NC(=O)c2nccc(C(F)(F)F)c2Cl)ccc1C]

In some embodiments R² is

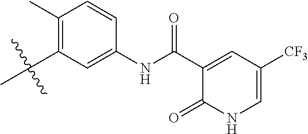 [SMILES: *c1cc(NC(=O)c2cc(C(F)(F)F)c[nH]c2=O)ccc1C]

In some embodiments R² is

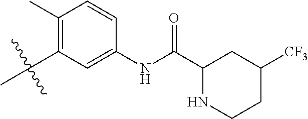 [SMILES: *c1cc(NC(=O)C2CC(C(F)(F)F)CCN2)ccc1C]

In some embodiments R² is

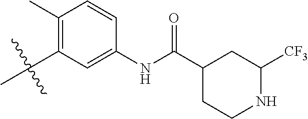 [SMILES: *c1cc(NC(=O)C2CCNC(C(F)(F)F)C2)ccc1C]

In some embodiments R² is

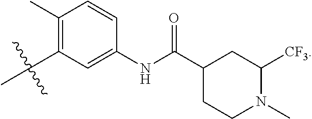 [SMILES: *c1cc(NC(=O)C2CCN(C)C(C(F)(F)F)C2)ccc1C]

In some embodiments R² is

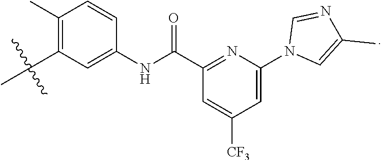

In some embodiments R²

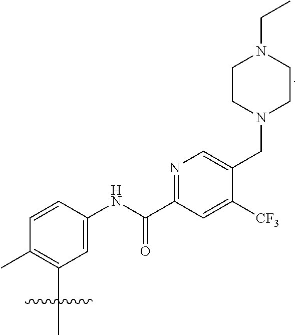 [SMILES: *c1cc(NC(=O)c2cc(C(F)(F)F)c(CN3CCN(CC)CC3)cn2)ccc1C]

In some embodiments R² is

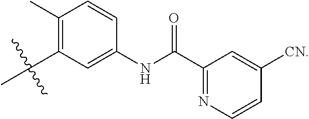 [SMILES: *c1cc(NC(=O)c2cc(C#N)ccn2)ccc1C]

In some embodiments R² is

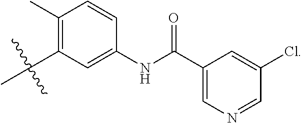 [SMILES: *c1cc(NC(=O)c2cncc(Cl)c2)ccc1C]

In some embodiments R² is

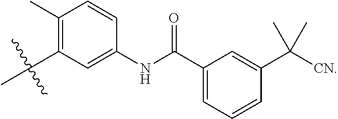 [SMILES: *c1cc(NC(=O)c2cccc(C(C)(C)C#N)c2)ccc1C]

In some embodiments R² is

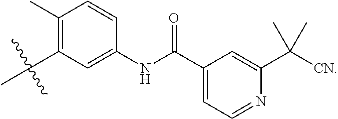 [SMILES: *c1cc(NC(=O)c2ccnc(C(C)(C)C#N)c2)ccc1C]

In some embodiments R² is

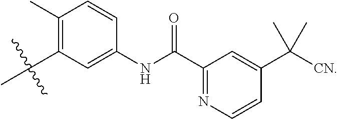 [SMILES: *c1cc(NC(=O)c2cc(C(C)(C)C#N)ccn2)ccc1C]

In some embodiments R² is

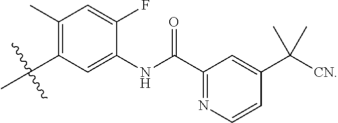 [SMILES: *c1cc(NC(=O)c2cc(C(C)(C)C#N)ccn2)c(F)cc1C]

In some embodiments R² is

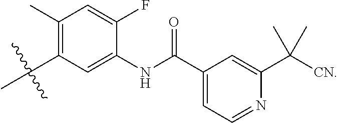 [SMILES: *c1cc(NC(=O)c2ccnc(C(C)(C)C#N)c2)c(F)cc1C]

In some embodiments R² is

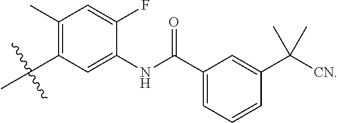 [SMILES: *c1cc(NC(=O)c2cccc(C(C)(C)C#N)c2)c(F)cc1C]

In some embodiments R² is

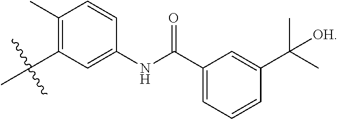 [SMILES: *c1cc(NC(=O)c2cccc(C(C)(C)O)c2)ccc1C]

In some embodiments R² is

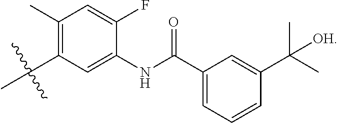 [SMILES: *c1cc(NC(=O)c2cccc(C(C)(C)O)c2)c(F)cc1C]

In some embodiments R² is

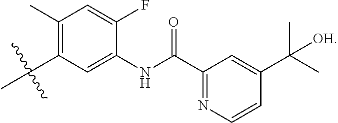 [SMILES: *c1cc(NC(=O)c2cc(C(C)(C)O)ccn2)c(F)cc1C]

In some embodiments R² is

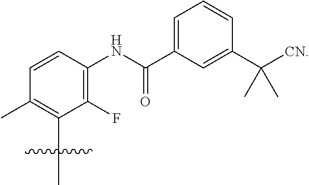 [SMILES: *c1c(C)ccc(NC(=O)c2cccc(C(C)(C)C#N)c2)c1F]

In some embodiments R² is

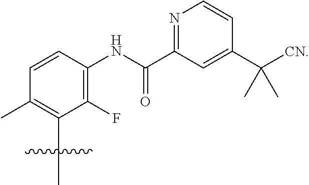 [SMILES: *c1c(C)ccc(NC(=O)c2cc(C(C)(C)C#N)ccn2)c1F]

In some embodiments R² is

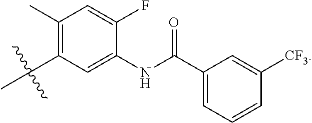 [SMILES: *c1cc(NC(=O)c2cccc(C(F)(F)F)c2)c(F)cc1C]

In some embodiments R² is

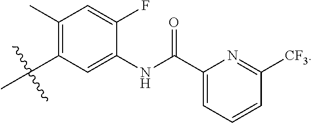 [SMILES: *c1cc(NC(=O)c2cccc(C(F)(F)F)n2)c(F)cc1C]

In some embodiments R² is

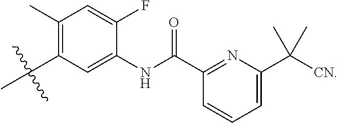 [SMILES: *c1cc(NC(=O)c2cccc(C(C)(C)C#N)n2)c(F)cc1C]

In some embodiments R² is

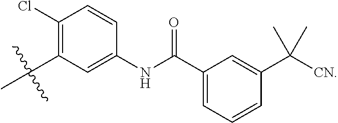 [SMILES: *c1cc(NC(=O)c2cccc(C(C)(C)C#N)c2)ccc1Cl]

In some embodiments R² is

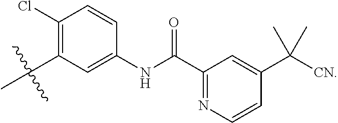 [SMILES: *c1cc(NC(=O)c2cc(C(C)(C)C#N)ccn2)ccc1Cl]

In some embodiments R² is

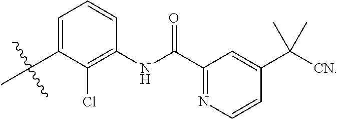 [SMILES: *c1cccc(NC(=O)c2cc(C(C)(C)C#N)ccn2)c1Cl]

In some embodiments R² is

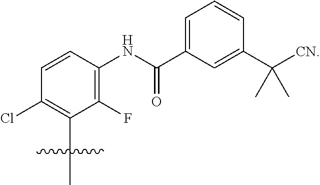 [SMILES: *c1c(Cl)ccc(NC(=O)c2cccc(C(C)(C)C#N)c2)c1F]

In some embodiments R² is

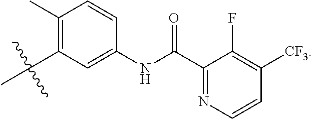 [SMILES: *c1cc(NC(=O)c2nccc(C(F)(F)F)c2F)ccc1C]

In some embodiments R² is

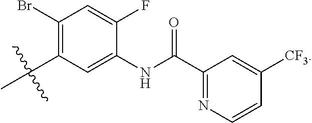 [SMILES: *c1cc(NC(=O)c2cc(C(F)(F)F)ccn2)c(F)cc1Br]

In some embodiments R² is

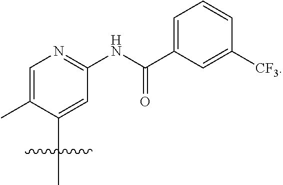 [SMILES: *c1cc(NC(=O)c2cccc(C(F)(F)F)c2)ncc1C]

In some embodiments R² is

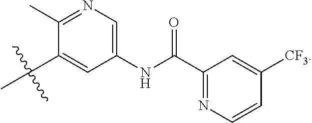 [SMILES: *c1cc(NC(=O)c2cc(C(F)(F)F)ccn2)cnc1C]

In some embodiments R² is

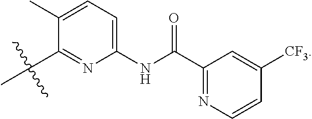 [SMILES: *c1nc(NC(=O)c2cc(C(F)(F)F)ccn2)ccc1C]

In some embodiments R² is

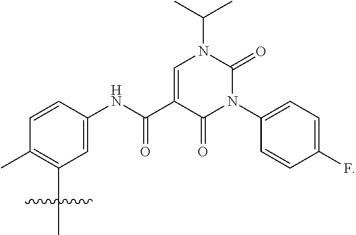 [SMILES: *c1cc(NC(=O)c2cn(C(C)C)c(=O)n(-c3ccc(F)cc3)c2=O)ccc1C]

In some embodiments R² is

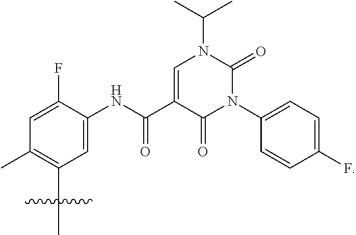 [SMILES: *c1cc(NC(=O)c2cn(C(C)C)c(=O)n(-c3ccc(F)cc3)c2=O)c(F)cc1C]

In some embodiments R² is

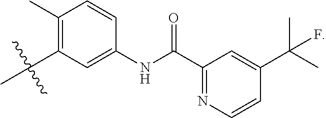 [SMILES: *c1cc(NC(=O)c2cc(C(C)(C)F)ccn2)ccc1C]

In some embodiments R² is

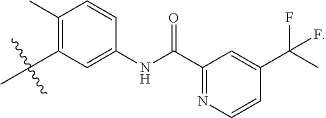 [SMILES: *c1cc(NC(=O)c2cc(C(C)(F)F)ccn2)ccc1C]

In some embodiments R² is

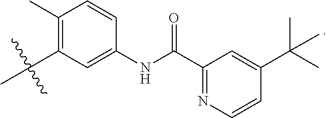 [SMILES: *c1cc(NC(=O)c2cc(C(C)(C)C)ccn2)ccc1C]

In some embodiments R² is

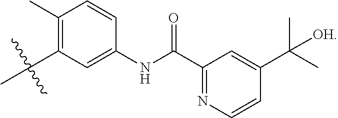 [SMILES: *c1cc(NC(=O)c2cc(C(C)(C)O)ccn2)ccc1C]

In some embodiments R² is

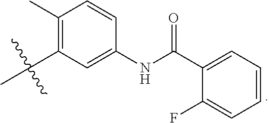 [SMILES: *c1cc(NC(=O)c2ccccc2F)ccc1C]

In some embodiments R² is

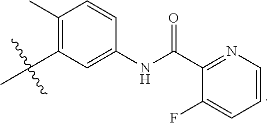 [SMILES: *c1cc(NC(=O)c2ncccc2F)ccc1C]

In some embodiments R² is

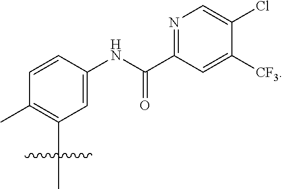 [SMILES: *c1cc(NC(=O)c2cc(C(F)(F)F)c(Cl)cn2)ccc1C]

In some embodiments R² is

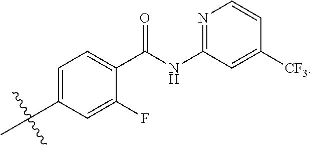 [SMILES: *c1ccc(C(=O)Nc2cc(C(F)(F)F)ccn2)c(F)c1]

In some embodiments R² is

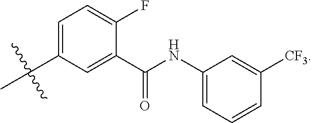 [SMILES: *c1ccc(F)c(C(=O)Nc2cccc(C(F)(F)F)c2)c1]

In some embodiments R² is

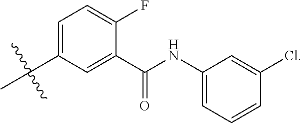 [SMILES: *c1ccc(F)c(C(=O)Nc2cccc(Cl)c2)c1]

In some embodiments R² is

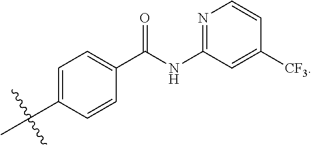 [SMILES: *c1ccc(C(=O)Nc2cc(C(F)(F)F)ccn2)cc1]

In some embodiments R² is

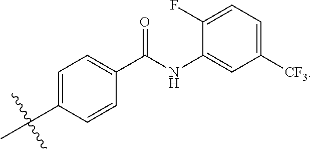 [SMILES: *c1ccc(C(=O)Nc2cc(C(F)(F)F)ccc2F)cc1]

In some embodiments R² is

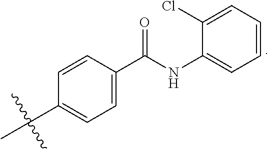 [SMILES: *c1ccc(C(=O)Nc2ccccc2Cl)cc1]

In some embodiments R² is

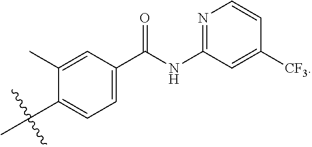 [SMILES: *c1ccc(C(=O)Nc2cc(C(F)(F)F)ccn2)cc1C]

In some embodiments R² is

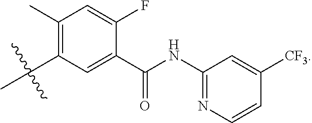 [SMILES: *c1cc(C(=O)Nc2cc(C(F)(F)F)ccn2)c(F)cc1C]

In some embodiments R² is

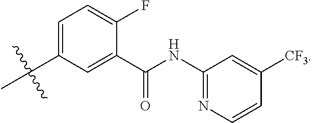 [SMILES: *c1ccc(F)c(C(=O)Nc2cc(C(F)(F)F)ccn2)c1]

In some embodiments R² is

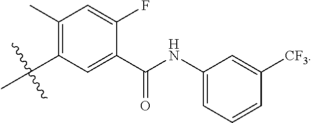 [SMILES: *c1cc(C(=O)Nc2cccc(C(F)(F)F)c2)c(F)cc1C]

In some embodiments R² is

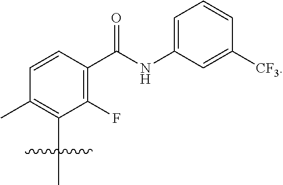 [SMILES: *c1c(C)ccc(C(=O)Nc2cccc(C(F)(F)F)c2)c1F]

In some embodiments R² is

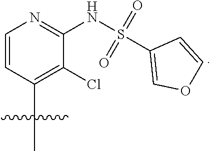 [SMILES: *c1ccnc(NS(=O)(=O)c2ccoc2)c1Cl]

In some embodiments R² is

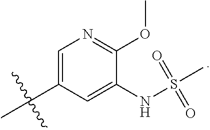 [SMILES: *c1cnc(OC)c(NS(C)(=O)=O)c1]

In some embodiments R² is

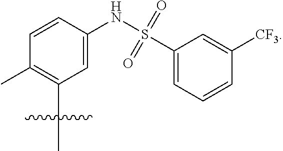 [SMILES: *c1cc(NS(=O)(=O)c2cccc(C(F)(F)F)c2)ccc1C]

In some embodiments R² is

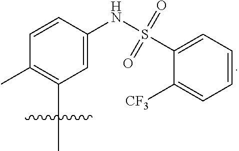 [SMILES: *c1cc(NS(=O)(=O)c2ccccc2C(F)(F)F)ccc1C]

In some embodiments R² is

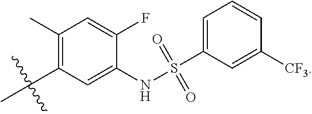 [SMILES: *c1cc(NS(=O)(=O)c2cccc(C(F)(F)F)c2)c(F)cc1C]

In some embodiments R² is

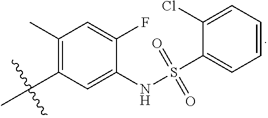 [SMILES: *c1cc(NS(=O)(=O)c2ccccc2Cl)c(F)cc1C]

In some embodiments R² is

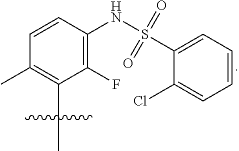 [SMILES: *c1c(C)ccc(NS(=O)(=O)c2ccccc2Cl)c1F]

In some embodiments R² is

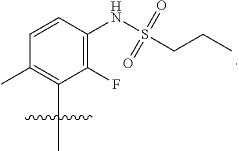 [SMILES: *c1c(C)ccc(NS(=O)(=O)CCC)c1F]

In some embodiments R² is

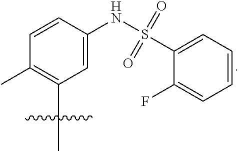 [SMILES: *c1cc(NS(=O)(=O)c2ccccc2F)ccc1C]

In some embodiments R² is

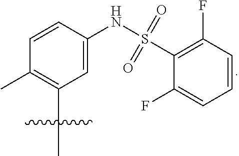 [SMILES: *c1cc(NS(=O)(=O)c2c(F)cccc2F)ccc1C]

In some embodiments R² is

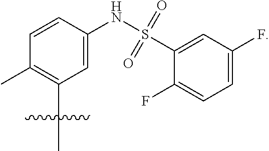 [SMILES: *c1cc(NS(=O)(=O)c2cc(F)ccc2F)ccc1C]

In some embodiments R² is

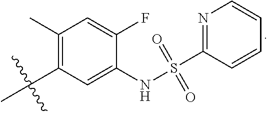 [SMILES: *c1cc(NS(=O)(=O)c2ccccn2)c(F)cc1C]

In some embodiments R² is

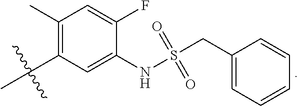 [SMILES: *c1cc(NS(=O)(=O)Cc2ccccc2)c(F)cc1C]

In some embodiments R² is

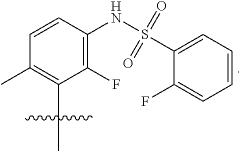 [SMILES: *c1c(C)ccc(NS(=O)(=O)c2ccccc2F)c1F]

In some embodiments R² is

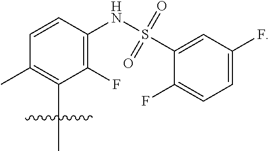 [SMILES: *c1c(C)ccc(NS(=O)(=O)c2cc(F)ccc2F)c1F]

In some embodiments R² is

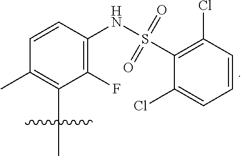 [SMILES: *c1c(C)ccc(NS(=O)(=O)c2c(Cl)cccc2Cl)c1F]

In some embodiments R² is

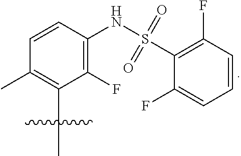 [SMILES: *c1c(C)ccc(NS(=O)(=O)c2c(F)cccc2F)c1F]

In some embodiments R² is

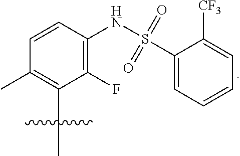 [SMILES: *c1c(C)ccc(NS(=O)(=O)c2ccccc2C(F)(F)F)c1F]

In some embodiments R² is

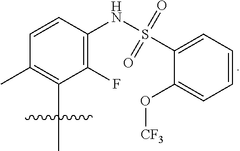 [SMILES: *c1c(C)ccc(NS(=O)(=O)c2ccccc2OC(F)(F)F)c1F]

In some embodiments R² is

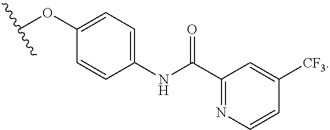 [SMILES: *Oc1ccc(NC(=O)c2cc(C(F)(F)F)ccn2)cc1]

In some embodiments R² is

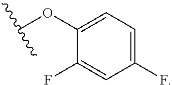 [SMILES: *Oc1ccc(F)cc1F]

In some embodiments R² is

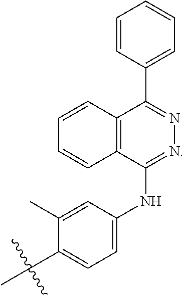 [SMILES: *c1ccc(Nc2nnc(-c3ccccc3)c3ccccc23)cc1C]

In some embodiments R² is

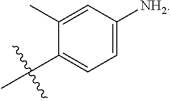 [SMILES: *c1ccc(N)cc1C]

In some embodiments R² is

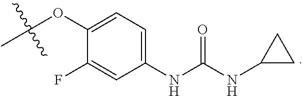 [SMILES: *Oc1ccc(NC(=O)NC2CC2)cc1F]

In some embodiments R² is

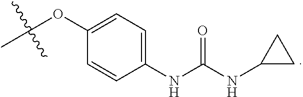 [SMILES: *Oc1ccc(NC(=O)NC2CC2)cc1]

In some embodiments R² is

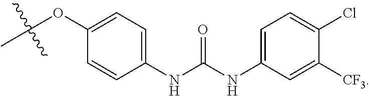 [SMILES: *Oc1ccc(NC(=O)Nc2ccc(Cl)c(C(F)(F)F)c2)cc1]

In some embodiments R² is

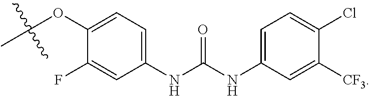 [SMILES: *Oc1ccc(NC(=O)Nc2ccc(Cl)c(C(F)(F)F)c2)cc1F]

In some embodiments R² is

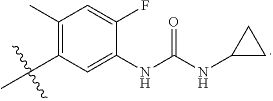 [SMILES: *c1cc(NC(=O)NC2CC2)c(F)cc1C]

In some embodiments R² is

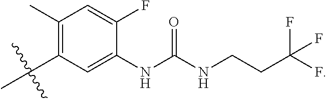 [SMILES: *c1cc(NC(=O)NCCC(F)(F)F)c(F)cc1C]

In some embodiments R² is

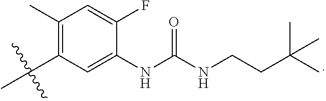 [SMILES: *c1cc(NC(=O)NCCC(C)(C)C)c(F)cc1C]

In some embodiments R² is

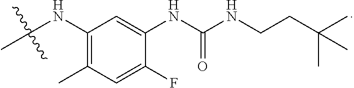 [SMILES: *Nc1cc(NC(=O)NCCC(C)(C)C)c(F)cc1C]

In some embodiments R² is

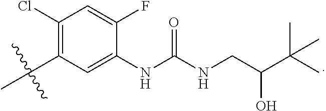 [SMILES: *c1cc(NC(=O)NCC(O)C(C)(C)C)c(F)cc1Cl]

In some embodiments R² is

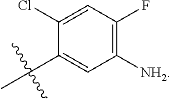 [SMILES: *c1cc(N)c(F)cc1Cl]

In some embodiments R² is

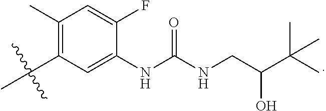 [SMILES: *c1cc(NC(=O)NCC(O)C(C)(C)C)c(F)cc1C]

In some embodiments R² is

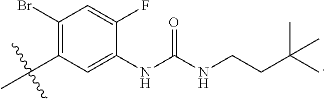 [SMILES: *c1cc(NC(=O)NCCC(C)(C)C)c(F)cc1Br]

In some embodiments R² is

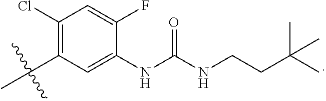 [SMILES: *c1cc(NC(=O)NCCC(C)(C)C)c(F)cc1Cl]

In some embodiments R² is

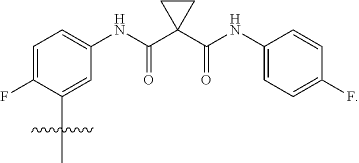 [SMILES: *c1cc(NC(=O)C2(C(=O)Nc3ccc(F)cc3)CC2)ccc1F]

In some embodiments R² is

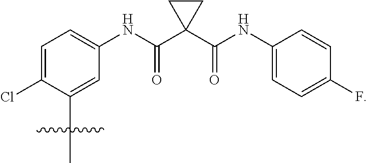 [SMILES: *c1cc(NC(=O)C2(C(=O)Nc3ccc(F)cc3)CC2)ccc1Cl]

In some embodiments R² is

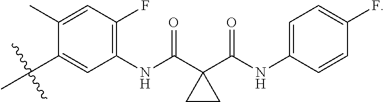 [SMILES: *c1cc(NC(=O)C2(C(=O)Nc3ccc(F)cc3)CC2)c(F)cc1C]

In some embodiments R² is

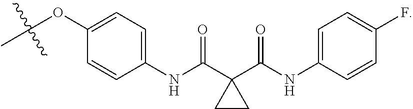 [SMILES: *Oc1ccc(NC(=O)C2(C(=O)Nc3ccc(F)cc3)CC2)cc1]

In some embodiments R² is

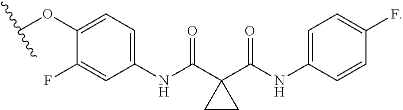 [SMILES: *Oc1ccc(NC(=O)C2(C(=O)Nc3ccc(F)cc3)CC2)cc1F]

In some embodiments of Formula (I), (I-1a), (I-2a), (I-2b), (I-3a), or (I-3b), R⁴ is selected from the group consisting of: (i) hydrogen; (ii) C₁-C₆ alkyl optionally substituted with one or more groups selected from the group consisting of halogen, C₁-C₆ alkoxy, hydroxyl, heteroaryl, and optionally substituted heterocyclyl; (iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl; (iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, and optionally substituted heterocyclyl; (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl; (vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and (vii) —SO₂(C₁-C₆ alkyl). In some embodiments, R⁴ is selected from the group consisting of: (i) hydrogen; (ii) C₁-C₆ alkyl optionally substituted with optionally substituted heterocyclyl; (iii) heterocyclyl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl; (iv) aryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl; and (v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl.

In some embodiments of Formula (I), (I-1a), (I-2a), (I-2b), (I-3a), or (I-3b), R⁴ is hydrogen.

In some embodiments of Formula (I), (I-1a), (I-2a), (I-2b), (I-3a), or (I-3b), R⁴ is C₁-C₆ alkyl optionally substituted with optionally substituted heterocyclyl.

In some embodiments of Formula (I), (I-1a), (I-2a), (I-2b), (I-3a), or (I-3b), R⁴ is heterocyclyl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl. In some embodiments, R⁴ is 3- to 12-membered heterocyclyl. In some embodiments, R⁴ is 3- to 12-membered heterocycloalkyl. In some embodiments, R⁴ is oxetanyl or tetrahydropyranyl. In some embodiments, R⁴ is 3-oxetanyl or 4-tetrahydropyranyl. In some embodiments, R⁴ is 3-oxetanyl. In some embodiments, R⁴ is 4-tetrahydropyranyl.

In some embodiments of Formula (I), (I-1a), (I-2a), (I-2b), (I-3a), or (I-3b), R⁴ is C₆-C₁₄ aryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted 3- to 12-membered heterocyclyl. In some embodiments, R⁴ is phenyl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted 5- to 6-membered heterocyclyl. In some embodiments, R⁴ is phenyl optionally substituted with halogen. In some embodiments, R⁴ is phenyl substituted with fluoro. In some embodiments, R⁴ is phenyl optionally substituted with hydroxyl. In some embodiments, R⁴ is phenyl substituted with optionally substituted C₁-C₆ alkyl. In some embodiments, R⁴ is phenyl substituted with C₁-C₆ alkyl which is further substituted with hydroxyl. In some embodiments, R⁴ is C₆-C₁₄ aryl substituted with halo and one additional substituent selected from the group consisting of hydroxyl, optionally substituted C₁-C₆ alkyl, optionally substituted C₁-C₆ alkoxy, optionally substituted C₁-C₆ thioalkoxy, and optionally substituted 3- to 12-membered heterocyclyl. In some embodiments, R⁴ is phenyl substituted with halo and one additional substituent selected from the group consisting of hydroxyl, optionally substituted C₁-C₆ alkyl, optionally substituted C₁-C₆ alkoxy, optionally substituted C₁-C₆ thioalkoxy, and optionally substituted 5- to 6-membered heterocyclyl. In some embodiments, R⁴ is phenyl substituted with fluoro and an optionally substituted 5- to 6-membered heterocyclyl. In certain embodiments, R⁴ is phenyl substituted with fluoro and an optionally substituted piperazinyl. In some embodiments, R⁴ is C₆-C₁₄ aryl substituted with optionally substituted C₁-C₆ alkoxy. In some embodiments, R⁴ is C₆-C₁₄ aryl substituted with C₁-C₆ alkoxy optionally substituted with —N(C₁-C₆ alkyl)₂. In some embodiments, R⁴ is phenyl substituted with C₁-C₆ alkoxy or C₁-C₆ thioalkoxy.

In some embodiments of Formula (I), (I-1a), (I-2a), (I-2b), (I-3a), or (I-3b), R⁴ is heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl. In some embodiments, R⁴ is 5- to 12-membered heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted 3- to 12-membered heterocyclyl. In some embodiments, R⁴ is 5- to 6-membered heteroaryl optionally substituted with one or more groups selected from the group consisting of halogen, C₁-C₆ alkyl, C₁-C₆ alkoxy, and optionally substituted 5- to 6-membered heterocyclyl. In some embodiments, R⁴ is 5- to 6-membered heteroaryl substituted with halo. In certain embodiments, R⁴ is pyridyl substituted with halo. In some embodiments, R⁴ is pyridyl. In some embodiments, R⁴ is 5- to 6-membered heteroaryl substituted with C₁-C₆ alkyl. In some embodiments, R⁴ is 5- to 6-membered heteroaryl substituted with C₁-C₆ alkoxy. In some embodiments, R⁴ is 5- to 6-membered heteroaryl substituted with C₁-hydroxyl.

In some embodiments of Formula (I), (I-1a), (I-2a), (I-2b), (I-3a), or (I-3b), R⁴ is selected from the group consisting of hydrogen, methyl, ethyl,

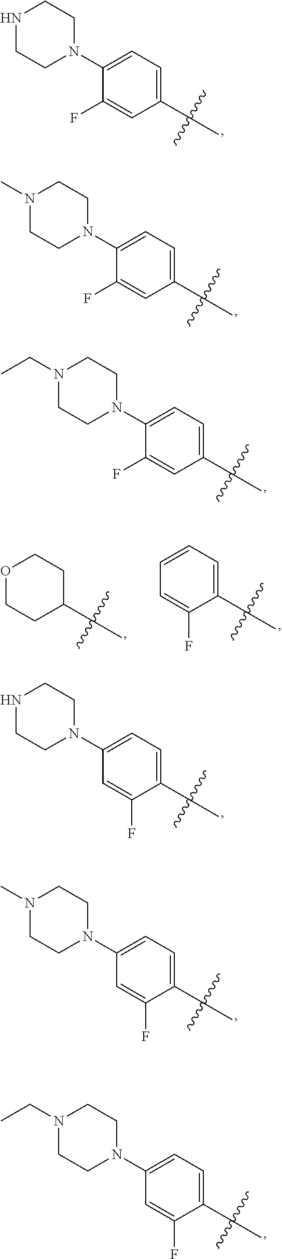

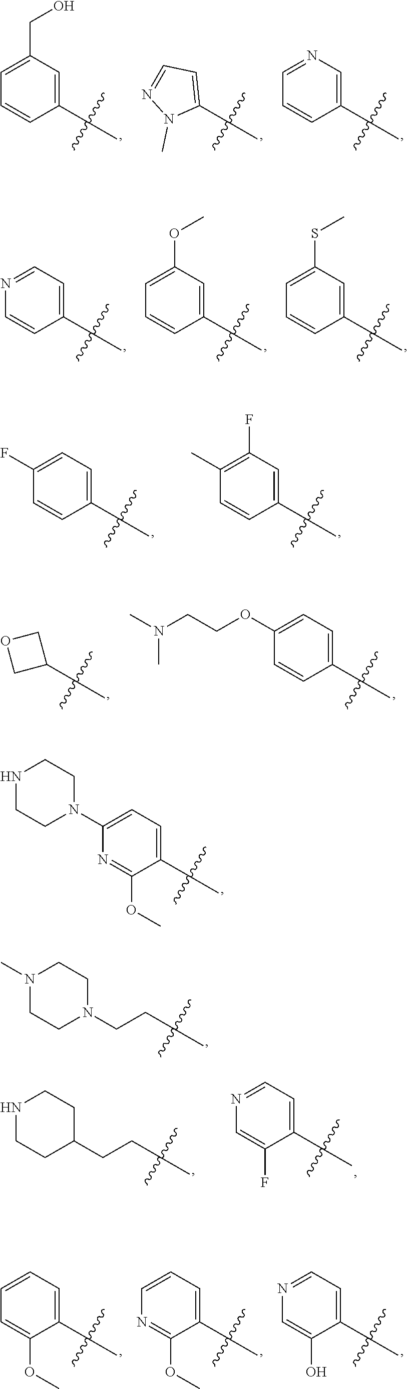

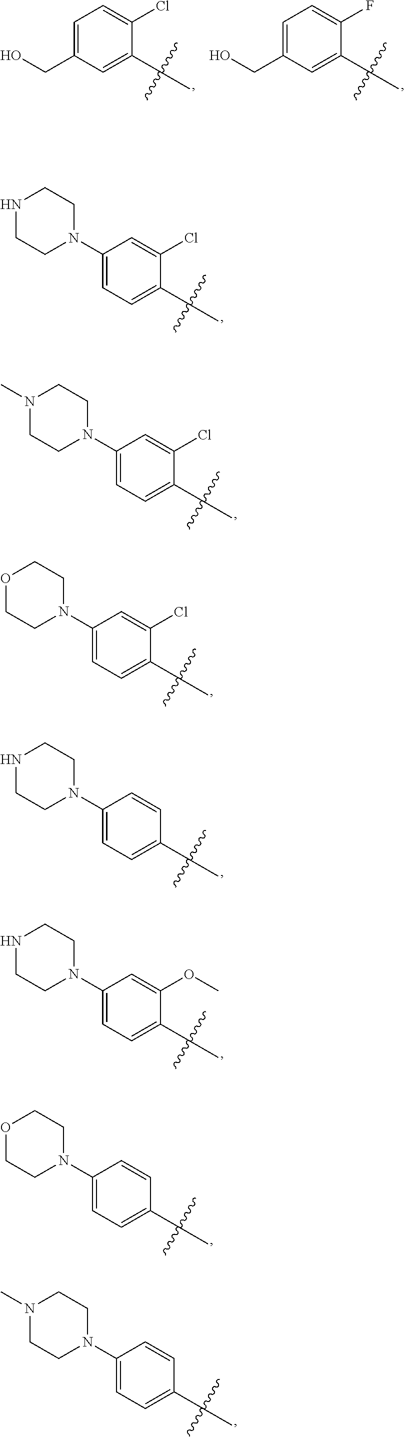

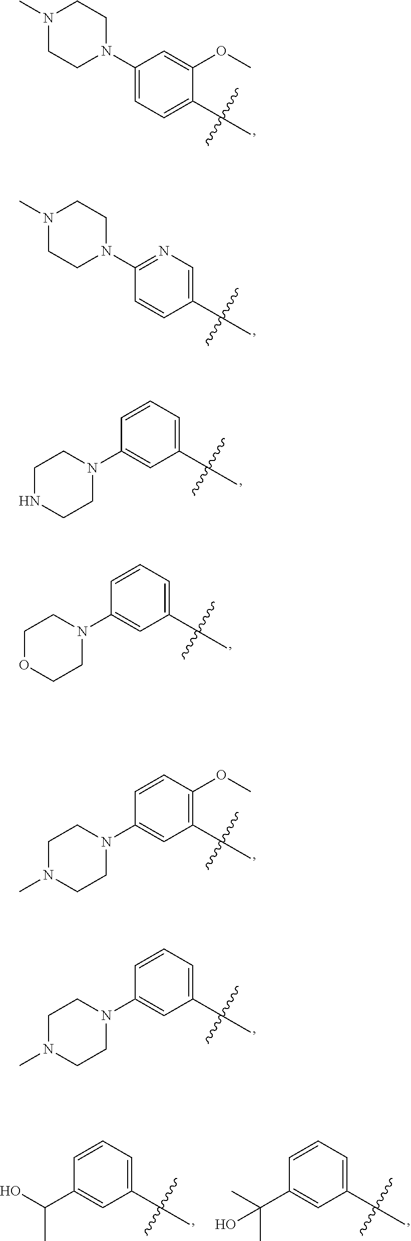

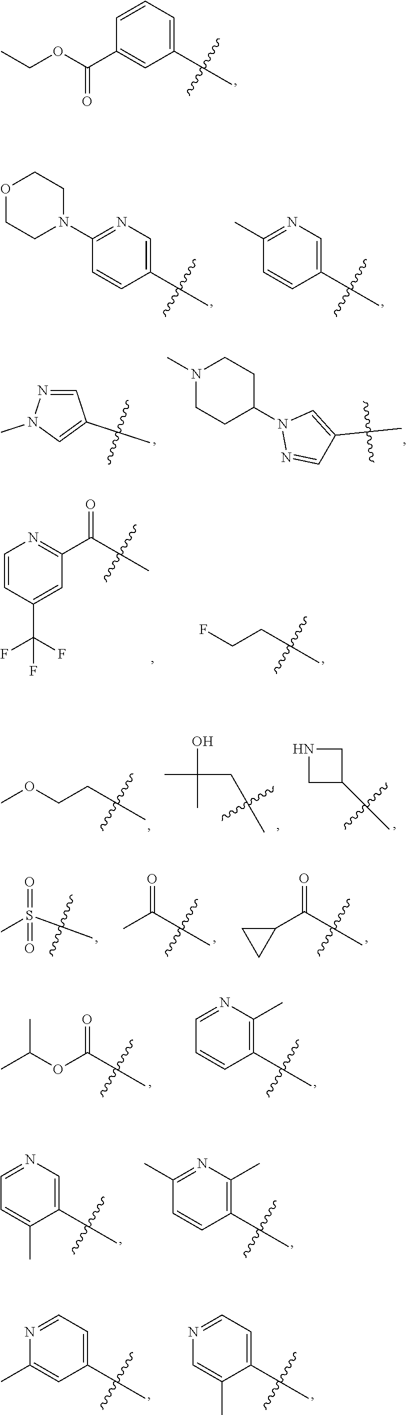

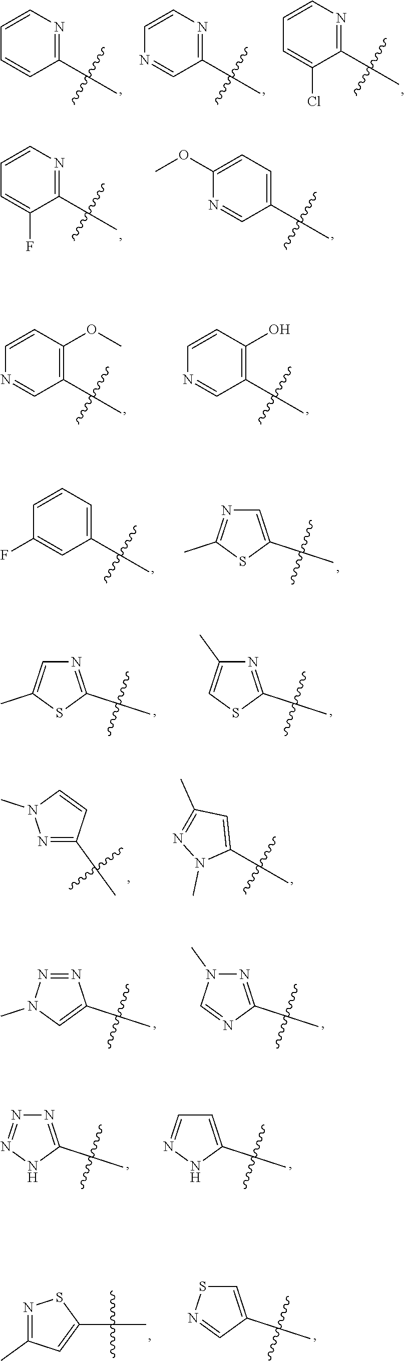

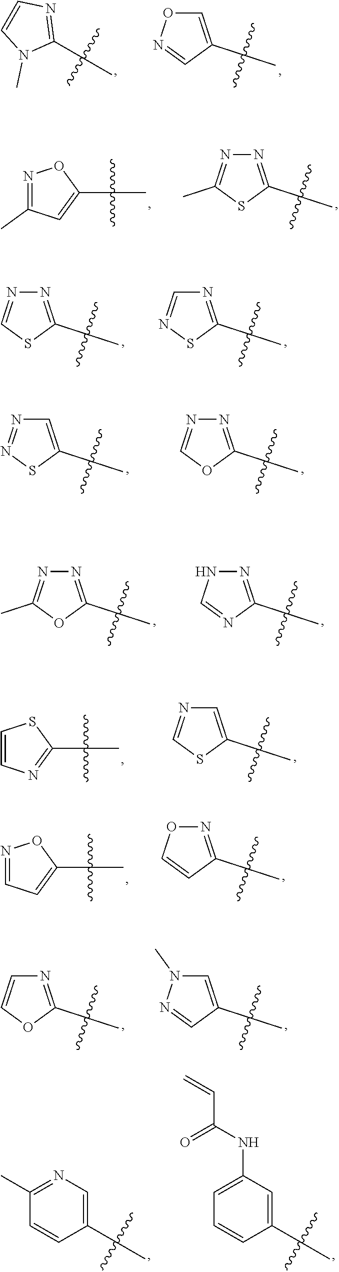

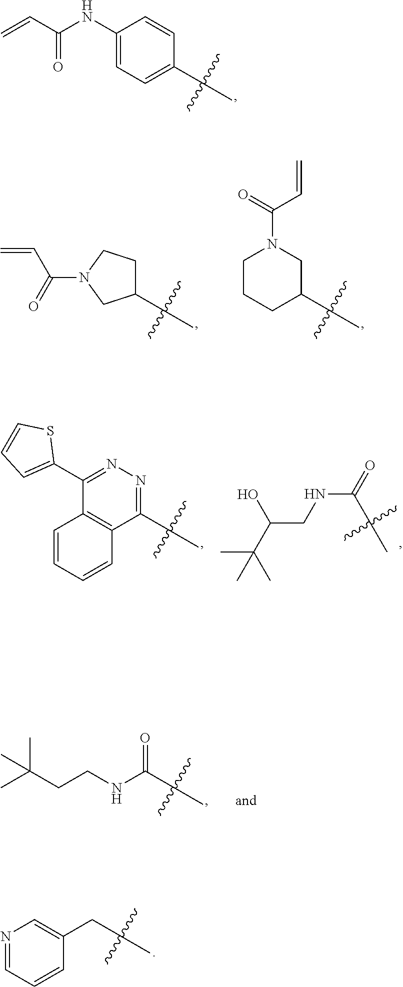

selected from the group consisting of hydrogen, methyl, ethyl,

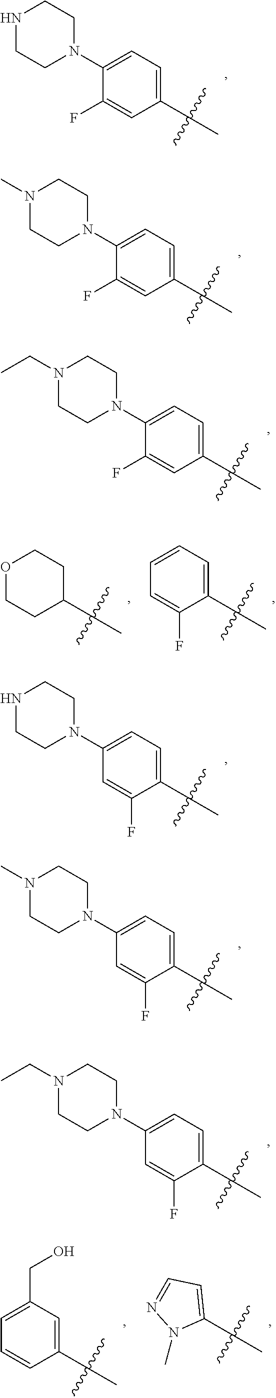

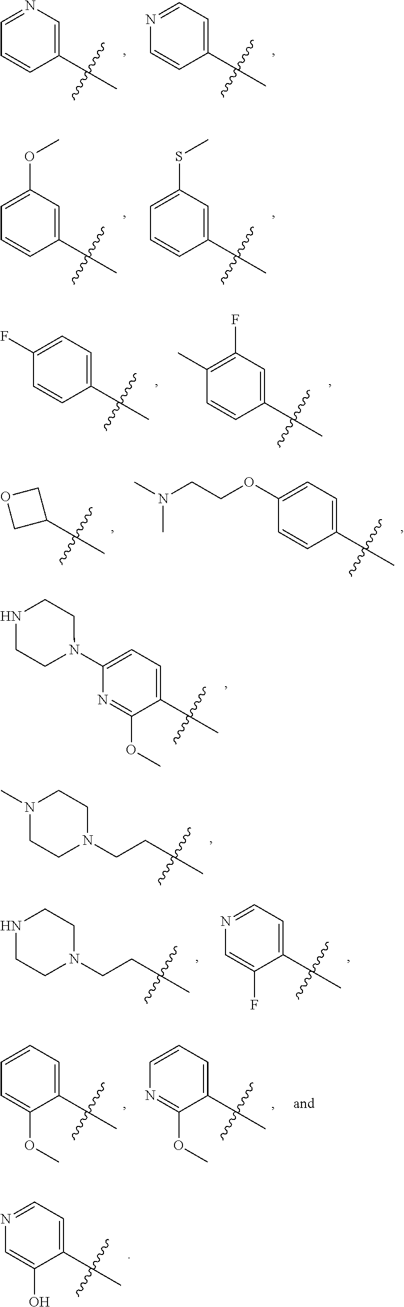

In some embodiments, R⁴ is

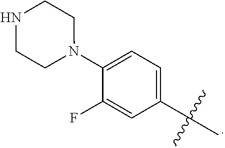 [SMILES: *c1ccc(N2CCNCC2)c(F)c1]

In some embodiments, R⁴ is

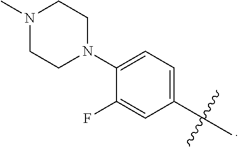 [SMILES: *c1ccc(N2CCN(C)CC2)c(F)c1]

In some embodiments, R⁴ is

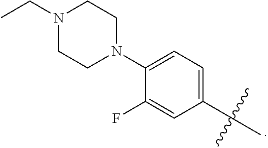 [SMILES: *c1ccc(N2CCN(CC)CC2)c(F)c1]

In some embodiments, R⁴ is

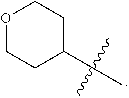 [SMILES: *C1CCOCC1]

In some embodiments, R⁴ is

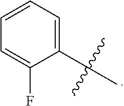 [SMILES: *c1ccccc1F]

In some embodiments, R⁴ is

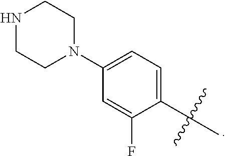 [SMILES: *c1ccc(N2CCNCC2)cc1F]

In some embodiments, R⁴ is

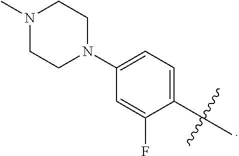 [SMILES: *c1ccc(N2CCN(C)CC2)cc1F]

In some embodiments, R⁴ is

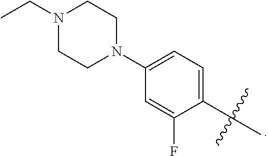 [SMILES: *c1ccc(N2CCN(CC)CC2)cc1F]

In some embodiments, R⁴ is

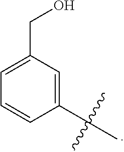 [SMILES: *c1cccc(CO)c1]

In some embodiments, R⁴ is

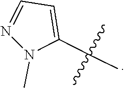 [SMILES: *c1ccnn1C]

In some embodiments, R⁴ is

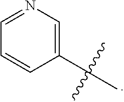 [SMILES: *c1cccnc1]

In some embodiments, R⁴ is

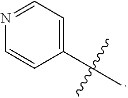 [SMILES: *c1ccncc1]

In some embodiments, R⁴ is

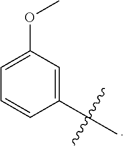 [SMILES: *c1cccc(OC)c1]

In some embodiments, R⁴ is

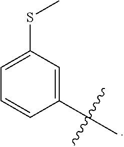 [SMILES: *c1cccc(SC)c1]

In some embodiments, R⁴ is

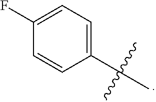 [SMILES: *c1ccc(F)cc1]

In some embodiments, R⁴ is

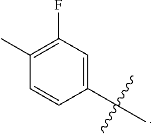 [SMILES: *c1ccc(C)c(F)c1]

In some embodiments, R⁴ is

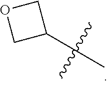 [SMILES: *C1COC1]

In some embodiments, R⁴ is

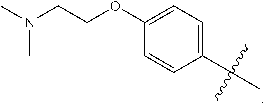 [SMILES: *c1ccc(OCCN(C)C)cc1]

In some embodiments, R⁴ is

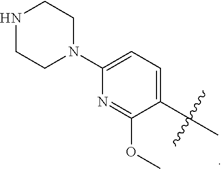 [SMILES: *c1ccc(N2CCNCC2)nc1OC]

In some embodiments, R⁴ is

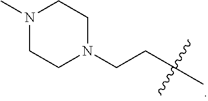 [SMILES: *CCN1CCN(C)CC1]

In some embodiments, R⁴ is

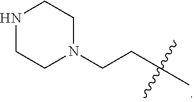 [SMILES: *CCN1CCNCC1]

In some embodiments, R⁴ is

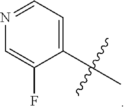 [SMILES: *c1ccncc1F]

In some embodiments, R⁴ is

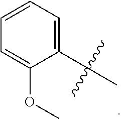 [SMILES: *c1ccccc1OC]

In some embodiments, R⁴ is

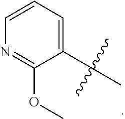 [SMILES: *c1cccnc1OC]

In some embodiments, R⁴ is

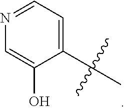 [SMILES: *c1ccncc1O]

In some embodiments, R⁴ is

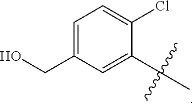 [SMILES: *c1cc(CO)ccc1Cl]

In some embodiments, R⁴ is

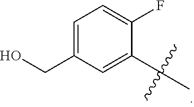 [SMILES: *c1cc(CO)ccc1F]

In some embodiments, R⁴ is

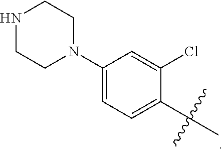 [SMILES: *c1ccc(N2CCNCC2)cc1Cl]

In some embodiments, R⁴ is

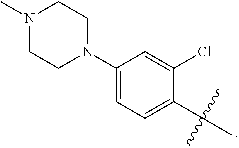 [SMILES: *c1ccc(N2CCN(C)CC2)cc1Cl]

In some embodiments, R⁴ is

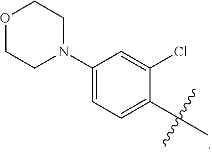 [SMILES: *c1ccc(N2CCOCC2)cc1Cl]

In some embodiments, R⁴ is

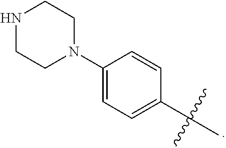 [SMILES: *c1ccc(N2CCNCC2)cc1]

In some embodiments, R⁴ is

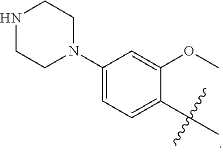 [SMILES: *c1ccc(N2CCNCC2)cc1OC]

In some embodiments, R⁴ is

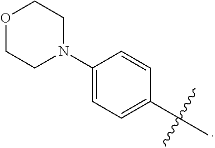 [SMILES: *c1ccc(N2CCOCC2)cc1]

In some embodiments, R⁴ is

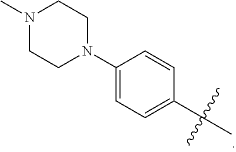 [SMILES: *c1ccc(N2CCN(C)CC2)cc1]

In some embodiments, R⁴ is

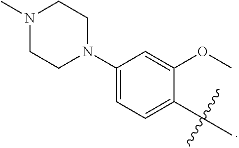

In some embodiments, R⁴ is

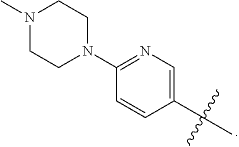 [SMILES: *c1ccc(N2CCN(C)CC2)nc1]

In some embodiments, R⁴ is

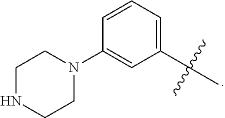 [SMILES: *c1cccc(N2CCNCC2)c1]

In some embodiments, R⁴ is

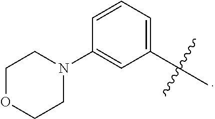 [SMILES: *c1cccc(N2CCOCC2)c1]

In some embodiments, R⁴ is

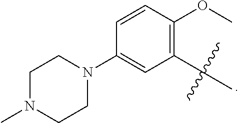

In some embodiments, R⁴ is

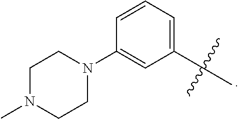 [SMILES: *c1cccc(N2CCN(C)CC2)c1]

In some embodiments, R⁴ is

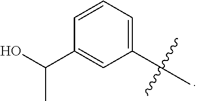 [SMILES: *c1cccc(C(C)O)c1]

In some embodiments, R⁴ is

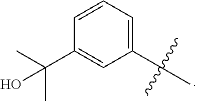 [SMILES: *c1cccc(C(C)(C)O)c1]

In some embodiments, R⁴ is

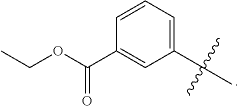 [SMILES: *c1cccc(C(=O)OCC)c1]

In some embodiments, R⁴ is

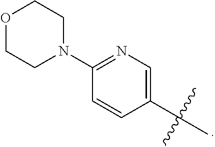 [SMILES: *c1ccc(N2CCOCC2)nc1]

In some embodiments, R⁴ is

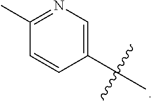 [SMILES: *c1ccc(C)nc1]

In some embodiments, R⁴ is

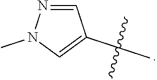 [SMILES: *c1cnn(C)c1]

In some embodiments, R⁴ is

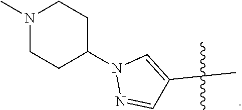 [SMILES: *c1cnn(C2CCN(C)CC2)c1]

In some embodiments, R⁴ is

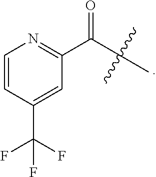 [SMILES: *C(=O)c1cc(C(F)(F)F)ccn1]

In some embodiments, R⁴ is

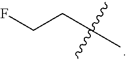 [SMILES: *CCF]

In some embodiments, R⁴ is

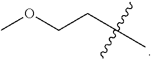 [SMILES: *CCOC]

In some embodiments, R⁴ is

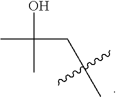 [SMILES: *CC(C)(C)O]

In some embodiments, R⁴ is

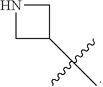 [SMILES: *C1CNC1]

In some embodiments, R⁴ is

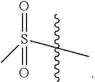 [SMILES: *S(C)(=O)=O]

In some embodiments, R⁴ is

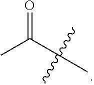 [SMILES: *C(C)=O]

In some embodiments R⁴ is

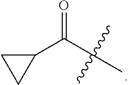 [SMILES: *C(=O)C1CC1]

In some embodiments, R⁴ is

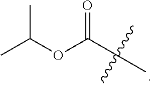 [SMILES: *C(=O)OC(C)C]

In some embodiments, R⁴ is

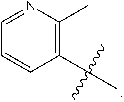 [SMILES: *c1cccnc1C]

In some embodiments, R⁴ is

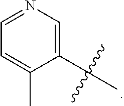 [SMILES: *c1cnccc1C]

In some embodiments, R⁴ is

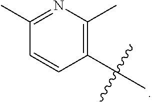 [SMILES: *c1ccc(C)nc1C]

In some embodiments, R⁴ is

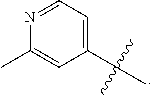 [SMILES: *c1ccnc(C)c1]

In some embodiments, R⁴ is

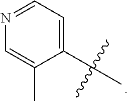 [SMILES: *c1ccncc1C]

In some embodiments, R⁴ is

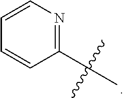 [SMILES: *c1ccccn1]

In some embodiments, R⁴ is

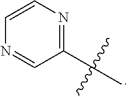 [SMILES: *c1cnccn1]

In some embodiments, R⁴ is

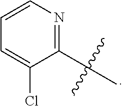 [SMILES: *c1ncccc1Cl]

In some embodiments, R⁴ is

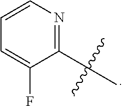 [SMILES: *c1ncccc1F]

In some embodiments, R⁴ is

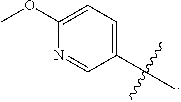 [SMILES: *c1ccc(OC)nc1]

In some embodiments, R⁴ is

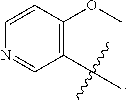 [SMILES: *c1cnccc1OC]

In some embodiments, R⁴ is

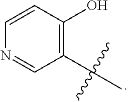 [SMILES: *c1cnccc1O]

In some embodiments, R⁴ is

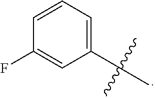 [SMILES: *c1cccc(F)c1]

In some embodiments, R⁴ is

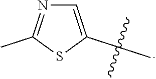 [SMILES: *c1cnc(C)s1]

In some embodiments, R⁴ is

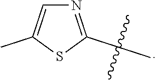 [SMILES: *c1ncc(C)s1]

In some embodiments, R⁴ is

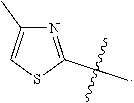 [SMILES: *c1nc(C)cs1]

In some embodiments, R⁴ is

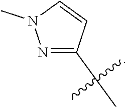 [SMILES: *c1ccn(C)n1]

In some embodiments, R⁴ is

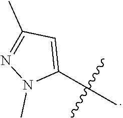 [SMILES: *c1cc(C)nn1C]

In some embodiments, R⁴ is

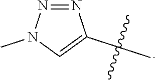 [SMILES: *c1cn(C)nn1]

In some embodiments, R⁴ is

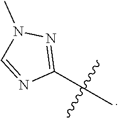 [SMILES: *c1ncn(C)n1]

In some embodiments, R⁴ is

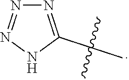 [SMILES: *c1nnn[nH]1]

In some embodiments, R⁴ is

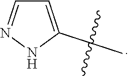 [SMILES: *c1ccn[nH]1]

In some embodiments, R⁴ is

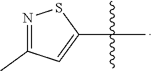 [SMILES: *c1cc(C)ns1]

In some embodiments, R⁴ is

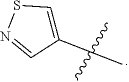 [SMILES: *c1cnsc1]

In some embodiments, R⁴ is

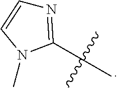 [SMILES: *c1nccn1C]

In some embodiments, R⁴ is

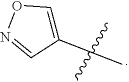 [SMILES: *c1cnoc1]

In some embodiments, R⁴ is

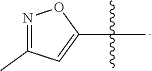 [SMILES: *c1cc(C)no1]

In some embodiments, R⁴ is

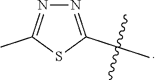 [SMILES: *c1nnc(C)s1]

In some embodiments, R⁴ is

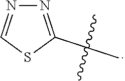 [SMILES: *c1nncs1]

In some embodiments, R⁴ is

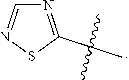 [SMILES: *c1ncns1]

In some embodiments, R⁴ is

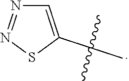 [SMILES: *c1cnns1]

In some embodiments, R⁴ is

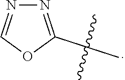 [SMILES: *c1nnco1]

In some embodiments, R⁴ is

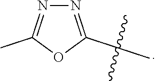 [SMILES: *c1nnc(C)o1]

In some embodiments, R⁴ is

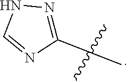 [SMILES: *c1nc[nH]n1]

In some embodiments, R⁴ is

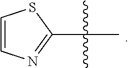 [SMILES: *c1nccs1]

In some embodiments, R⁴ is

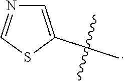 [SMILES: *c1cncs1]

In some embodiments, R⁴ is

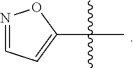 [SMILES: *c1ccno1]

In some embodiments, R⁴ is

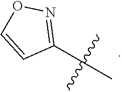 [SMILES: *c1ccon1]

In some embodiments, R⁴ is

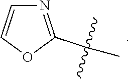 [SMILES: *c1ncco1]

In some embodiments, R⁴ is

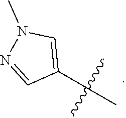 [SMILES: *c1cnn(C)c1]

In some embodiments, R⁴ is

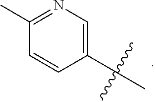 [SMILES: *c1ccc(C)nc1]

In some embodiments, R⁴ is

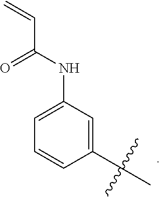 [SMILES: *c1cccc(NC(=O)C=C)c1]

In some embodiments, R⁴ is

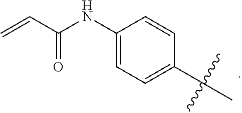 [SMILES: *c1ccc(NC(=O)C=C)cc1]

In some embodiments, R⁴ is

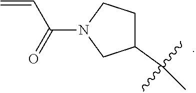 [SMILES: *C1CCN(C(=O)C=C)C1]

In some embodiments, R⁴ is

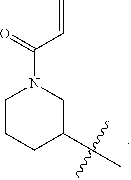 [SMILES: *C1CCCN(C(=O)C=C)C1]

In some embodiments, R⁴ is

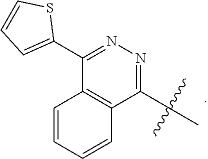 [SMILES: *c1nnc(-c2cccs2)c2ccccc12]

In some embodiments, R⁴ is

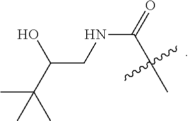 [SMILES: *C(=O)NCC(O)C(C)(C)C]

In some embodiments, R⁴ is

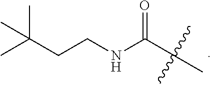 [SMILES: *C(=O)NCCC(C)(C)C]

In some embodiments, R⁴ is

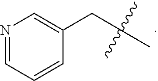 [SMILES: *Cc1cccnc1]

In some embodiments of Formula (I), (I-1a), (I-2a), (I-2b), (I-3a), or (I-3b), R⁵ is hydrogen. In some embodiments, R⁵ is C₁-C₆ alkyl. In some embodiments, R⁵ is methyl. In some embodiments, R⁵ is heterocyclyl. In some embodiments, R⁵ is 3-oxetanyl.

In some embodiments of Formula (I), (I-1a), (I-2a), (I-2b), (I-3a), or (I-3b), R⁴ and R⁵ are taken together with the nitrogen to which they are attached to form an optionally substituted heterocyclyl. In some embodiments, R⁴ and R⁵ are taken together with the nitrogen to which they are attached to form a substituted or unsubstituted 5- to 6-membered heterocyclyl. In certain embodiments, R⁴ and R⁵ are taken together with the nitrogen to which they are attached to form piperazinyl optionally substituted with 5- to 6-membered heteroaryl. In some embodiments of Formula (I), (I-1a), (I-2a), (I-2b), (I-3a), or (I-3b),

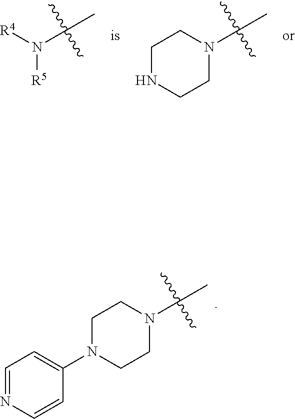

In some embodiments, R⁴ and R⁵ are taken together with the nitrogen to which they are attached to form an optionally substituted heteroaryl. In some embodiments, R⁴ and R⁵ are taken together with the nitrogen to which they are attached to form an optionally substituted pyrazole. In some embodiments, R⁴ and R⁵ are taken together with the nitrogen to which they are attached to form an optionally substituted heteroaryl. In some embodiments, R⁴ and R⁵ are taken together with the nitrogen to which they are attached to form 5-amino-1H-pyrazol-1-yl.

Any of the embodiments detailed herein with respect to Formula (I), where applicable, apply equally to Formula (I-1a), (I-2a), (I-2b), (I-3a), and (I-3b). It is also understood that the descriptions of any variable of Formula (I), (I-1a), (I-2a), (I-2b), (I-3a), or (I-3b) may, where applicable, be combined with one or more descriptions of any other variable, the same as if each and every combination of variables were specifically and individually listed. For example, every description of R² may be combined with every description of G¹, G², R¹, R³, R⁴, R⁵, m, and n the same as if each and every combination were specifically and individually listed. Likewise, every description of R⁴ may be combined with every description of G¹, G², R¹, R², R³, R⁵, m, and n the same as if each and every description were specifically and individually listed.

In one variation, compounds of the formulae provided herein contain one or more of the following structural features: (i) G¹ is N; (ii) G² is CH; (iii) m is 0; (iv) R³ is hydrogen; and (v) R⁵ is hydrogen.

In some embodiments, provided herein are compounds of Formula (I), (I-1a), (I-2a), (I-2b), (I-3a), and (I-3b), or pharmaceutically acceptable salts thereof.

In some embodiments, provided herein are compounds and salts thereof described in Table 1, and uses thereof.

TABLE 1

| Cpd | | |
|---|---|---|
| No | Structure | Chemical Name |
| 1 | 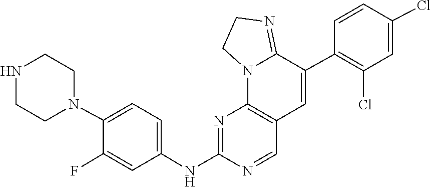 [SMILES: Fc1cc(Nc2ncc3c(n2)N2CCN=C2C(c2ccc(Cl)cc2Cl)=C3)ccc1N1CCNCC1] | 6-(2,4-dichlorophenyl)-N-(3- fluoro-4-(piperazin-1- yl)phenyl)-8,9- dihydroimidazo[1′,2′:1,6] pyrido[2,3-d]pyrimidin-2-amine |
| 2 | 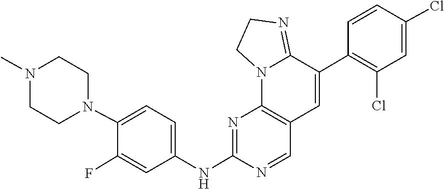 [SMILES: CN1CCN(c2ccc(Nc3ncc4c(n3)N3CCN=C3C(c3ccc(Cl)cc3Cl)=C4)cc2F)CC1] | 6-(2,4-dichlorophenyl)-N-(3- fluoro-4-(4-methylpiperazin- 1-yl)phenyl)-8,9- dihydroimidazo[1′,2′:1,6] pyrido[2,3-d]pyrimidin-2-amine |
| 3 | 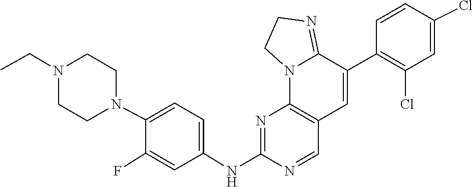 [SMILES: CCN1CCN(c2ccc(Nc3ncc4c(n3)N3CCN=C3C(c3ccc(Cl)cc3Cl)=C4)cc2F)CC1] | 6-(2,4-dichlorophenyl)-N-(4- (4-ethylpiperazin-1-yl)-3- fluorophenyl)-8,9- dihydroimidazo[1′,2′:1,6] pyrido[2,3-d]pyrimidin-2-amine |
| 4 | 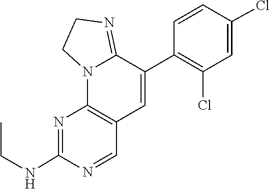 [SMILES: CCNc1ncc2c(n1)N1CCN=C1C(c1ccc(Cl)cc1Cl)=C2] | 6-(2,4-dichlorophenyl)-N- ethyl-8,9- dihydroimidazo[1′,2′:1,6] pyrido[2,3-d]pyrimidin-2-amine |
| 5 | 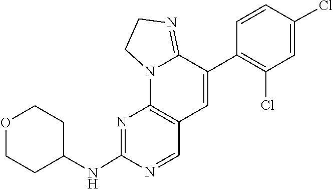 [SMILES: Clc1ccc(C2=Cc3cnc(NC4CCOCC4)nc3N3CCN=C23)c(Cl)c1] | 6-(2,4-dichlorophenyl)-N- (tetrahydro-2H-pyran-4-yl)- 8,9- dihydroimidazo[1′,2′:1,6] pyrido[2,3-d]pyrimidin-2-amine |
| 6 | 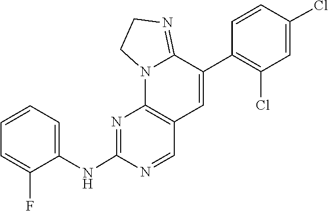 [SMILES: Fc1ccccc1Nc1ncc2c(n1)N1CCN=C1C(c1ccc(Cl)cc1Cl)=C2] | 6-(2,4-dichlorophenyl)-N-(2- fluorophenyl)-8,9- dihydroimidazo[1′,2′:1,6] pyrido[2,3-d]pyrimidin-2-amine |
| 7 | 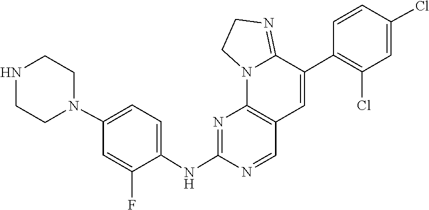 [SMILES: Fc1cc(N2CCNCC2)ccc1Nc1ncc2c(n1)N1CCN=C1C(c1ccc(Cl)cc1Cl)=C2] | 6-(2,4-dichlorophenyl)-N-(2- fluoro-4-(piperazin-1- yl)phenyl)-8,9- dihydroimidazo[1′,2′:1,6] pyrido[2,3-d]pyrimidin-2-amine |
| 8 | 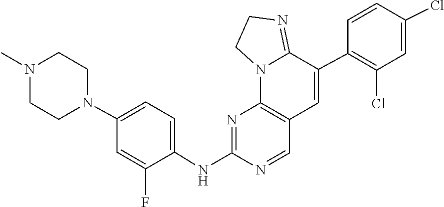 [SMILES: CN1CCN(c2ccc(Nc3ncc4c(n3)N3CCN=C3C(c3ccc(Cl)cc3Cl)=C4)c(F)c2)CC1] | 6-(2,4-dichlorophenyl)-N-(2- fluoro-4-(4-methylpiperazin- 1-yl)phenyl)-8,9- dihydroimidazo[1′,2′:1,6] pyrido[2,3-d]pyrimidin-2-amine |
| 9 | 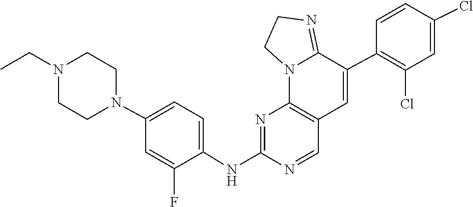 [SMILES: CCN1CCN(c2ccc(Nc3ncc4c(n3)N3CCN=C3C(c3ccc(Cl)cc3Cl)=C4)c(F)c2)CC1] | 6-(2,4-dichlorophenyl)-N-(4- (4-ethylpiperazin-1-yl)-2- fluorophenyl)-8,9- dihydroimidazo[1′,2′:1,6] pyrido[2,3-d]pyrimidin-2-amine |
| 10 | 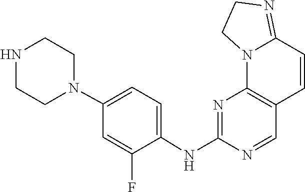 [SMILES: Fc1cc(N2CCNCC2)ccc1Nc1ncc2c(n1)N1CCN=C1C=C2] | N-(2-fluoro-4-(piperazin-1- yl)phenyl)-8,9- dihydroimidazo[1′,2′:1,6] pyrido[2,3-d]pyrimidin-2-amine |
| 11 | 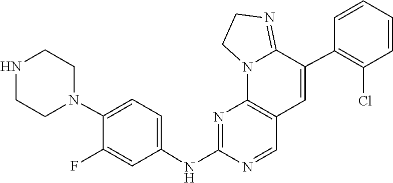 [SMILES: Fc1cc(Nc2ncc3c(n2)N2CCN=C2C(c2ccccc2Cl)=C3)ccc1N1CCNCC1] | 6-(2-chlorophenyl)-N-(3- fluoro-4-(piperazin-1- yl)phenyl)-8,9- dihydroimidazo[1′,2′:1,6] pyrido[2,3-d]pyrimidin-2-amine |
| 12 | 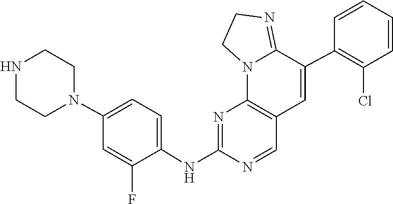 [SMILES: Fc1cc(N2CCNCC2)ccc1Nc1ncc2c(n1)N1CCN=C1C(c1ccccc1Cl)=C2] | 6-(2-chlorophenyl)-N-(2- fluoro-4-(piperazin-1- yl)phenyl)-8,9- dihydroimidazo[1′,2′:1,6] pyrido[2,3-d]pyrimidin-2-amine |
| 13 | 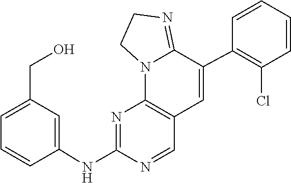 [SMILES: OCc1cccc(Nc2ncc3c(n2)N2CCN=C2C(c2ccccc2Cl)=C3)c1] | (3-((6-(2-chlorophenyl)-8,9- dihydroimidazo[1′,2′:1,6] pyrido[2,3-d]pyrimidin-2- yl)amino)phenyl)methanol |
| 14 | 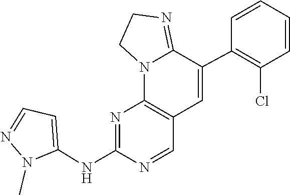 [SMILES: Cn1nccc1Nc1ncc2c(n1)N1CCN=C1C(c1ccccc1Cl)=C2] | 6-(2-chlorophenyl)-N-(1- methyl-1H-pyrazol-5-yl)-8,9- dihydroimidazo[1′,2′:1,6] pyrido[2,3-d]pyrimidin-2-amine |
| 15 | 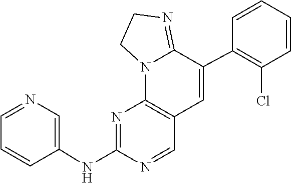 [SMILES: Clc1ccccc1C1=Cc2cnc(Nc3cccnc3)nc2N2CCN=C12] | 6-(2-chlorophenyl)-N- (pyridin-3-yl)-8,9- dihydroimidazo[1′,2′:1,6] pyrido[2,3-d]pyrimidin-2-amine |
| 16 | 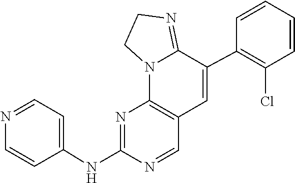 [SMILES: Clc1ccccc1C1=Cc2cnc(Nc3ccncc3)nc2N2CCN=C12] | 6-(2-chlorophenyl)-N- (pyridin-4-yl)-8,9- dihydroimidazo[1′,2′:1,6] pyrido[2,3-d]pyrimidin-2-amine |
| 17 | 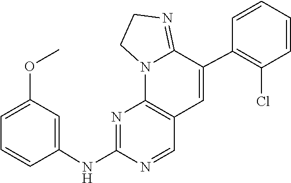 [SMILES: COc1cccc(Nc2ncc3c(n2)N2CCN=C2C(c2ccccc2Cl)=C3)c1] | 6-(2-chlorophenyl)-N-(3- methoxyphenyl)-8,9- dihydroimidazo[1′,2′:1,6] pyrido[2,3-d]pyrimidin-2-amine |
| 18 | 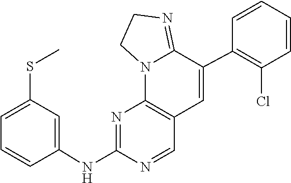 [SMILES: CSc1cccc(Nc2ncc3c(n2)N2CCN=C2C(c2ccccc2Cl)=C3)c1] | 6-(2-chlorophenyl)-N-(3- (methylthio)phenyl)-8,9- dihydroimidazo[1′,2′:1,6] pyrido[2,3-d]pyrimidin-2-amine |
| 19 | 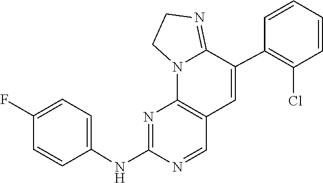 [SMILES: Fc1ccc(Nc2ncc3c(n2)N2CCN=C2C(c2ccccc2Cl)=C3)cc1] | 6-(2-chlorophenyl)-N-(4- fluorophenyl)-8,9- dihydroimidazo[1′,2′:1,6] pyrido[2,3-d]pyrimidin-2-amine |
| 20 | 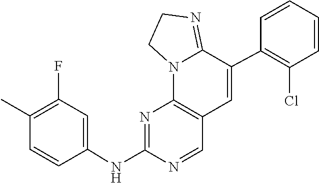 [SMILES: Cc1ccc(Nc2ncc3c(n2)N2CCN=C2C(c2ccccc2Cl)=C3)cc1F] | 6-(2-chlorophenyl)-N-(3- fluoro-4-methylpheny1)-8,9- dihydroimidazo[1′,2′:1,6] pyrido[2,3-d]pyrimidin-2-amine |
| 21 | 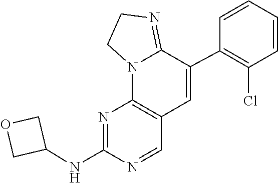 [SMILES: Clc1ccccc1C1=Cc2cnc(NC3COC3)nc2N2CCN=C12] | 6-(2-chlorophenyl)-N- (oxetan-3-yl)-8,9- dihydroimidazo[1′,2′:1,6] pyrido[2,3-d]pyrimidin-2-amine |
| 22 | 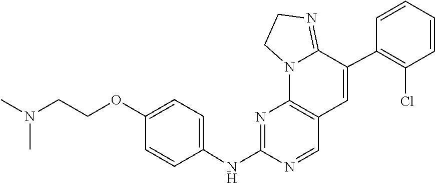 [SMILES: CN(C)CCOc1ccc(Nc2ncc3c(n2)N2CCN=C2C(c2ccccc2Cl)=C3)cc1] | 6-(2-chlorophenyl)-N-(4-(2- (dimethylamino)ethoxy) phenyl)-8,9- dihydroimidazo[1′,2′:1,6] pyrido[2,3-d]pyrimidin-2-amine |
| 23 | 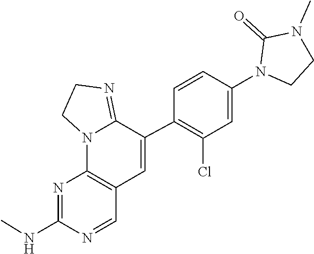 [SMILES: CNc1ncc2c(n1)N1CCN=C1C(c1ccc(N3CCN(C)C3=O)cc1Cl)=C2] | 1-(3-chloro-4-(2- (methylamino)-8,9- dihydroimidazo[1′,2′:1,6] pyrido[2,3-d]pyrimidin-6- yl)phenyl)-3- methylimidazolidin-2-one |
| 24 | 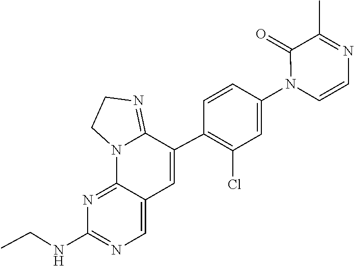 [SMILES: CCNc1ncc2c(n1)N1CCN=C1C(c1ccc(-n3ccnc(C)c3=O)cc1Cl)=C2] | 1-(3-chloro-4-(2- (ethylamino)-8,9- dihydroimidazo[1′,2′:1,6] pyrido[2,3-d]pyrimidin-6- yl)phenyl)-3-methylpyrazin- 2(1H)-one |
| 25 | 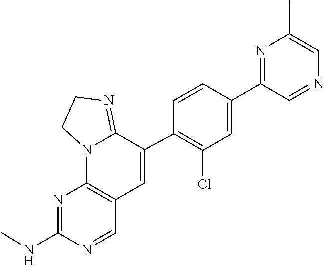 [SMILES: CNc1ncc2c(n1)N1CCN=C1C(c1ccc(-c3cncc(C)n3)cc1Cl)=C2] | 6-(2-chloro-4-(6- methylpyrazin-2-yl)phenyl)- N-methyl-8,9- dihydroimidazo[1′,2′:1,6] pyrido[2,3-d]pyrimidin-2-amine |
| 26 | 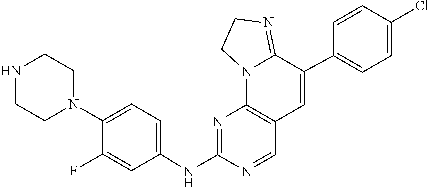 [SMILES: Fc1cc(Nc2ncc3c(n2)N2CCN=C2C(c2ccc(Cl)cc2)=C3)ccc1N1CCNCC1] | 6-(4-chlorophenyl)-N-(3- fluoro-4-(piperazin-1- yl)phenyl)-8,9- dihydroimidazo[1′,2′:1,6] pyrido[2,3-d]pyrimidin-2-amine |
| 27 | 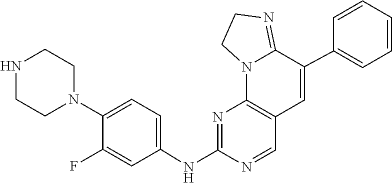 [SMILES: Fc1cc(Nc2ncc3c(n2)N2CCN=C2C(c2ccccc2)=C3)ccc1N1CCNCC1] | N-(3-fluoro-4-(piperazin-1- yl)phenyl)-6-phenyl-8,9- dihydroimidazo[1′,2′:1,6] pyrido[2,3-d]pyrimidin-2-amine |
| 28 | 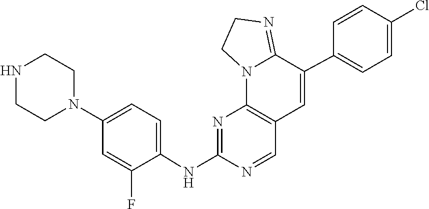 [SMILES: Fc1cc(N2CCNCC2)ccc1Nc1ncc2c(n1)N1CCN=C1C(c1ccc(Cl)cc1)=C2] | 6-(4-chlorophenyl)-N-(2- fluoro-4-(piperazin-1- yl)phenyl)-8,9- dihydroimidazo[1′,2′:1,6] pyrido[2,3-d]pyrimidin-2-amine |
| 29 | 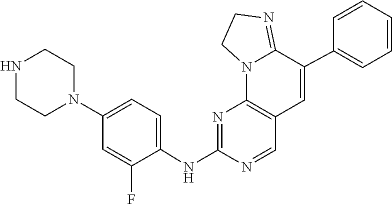 [SMILES: Fc1cc(N2CCNCC2)ccc1Nc1ncc2c(n1)N1CCN=C1C(c1ccccc1)=C2] | N-(2-fluoro-4-(piperazin-1- yl)phenyl)-6-phenyl-8,9- dihydroimidazo[1′,2′:1,6] pyrido[2,3-d]pyrimidin-2-amine |
| 30 | 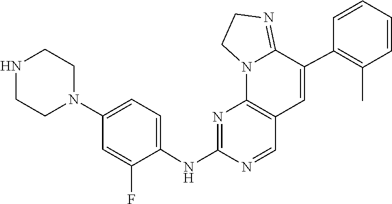 [SMILES: Cc1ccccc1C1=Cc2cnc(Nc3ccc(N4CCNCC4)cc3F)nc2N2CCN=C12] | N-(2-fluoro-4-(piperazin-1- yl)phenyl)-6-(o-toly1)-8,9- dihydroimidazo[1′,2′:1,6] pyrido[2,3-d]pyrimidin-2-amine |
| 31 | 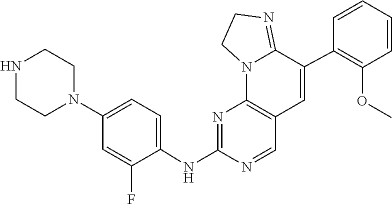 [SMILES: COc1ccccc1C1=Cc2cnc(Nc3ccc(N4CCNCC4)cc3F)nc2N2CCN=C12] | N-(2-fluoro-4-(piperazin-1- yl)phenyl)-6-(2- methoxyphenyl)-8,9- dihydroimidazo[1′,2′:1,6] pyrido[2,3-d]pyrimidin-2-amine |
| 32 | 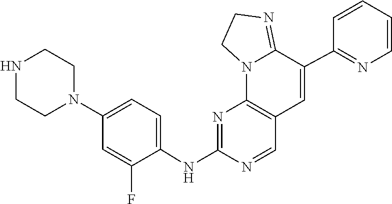 [SMILES: Fc1cc(N2CCNCC2)ccc1Nc1ncc2c(n1)N1CCN=C1C(c1ccccn1)=C2] | N-(2-fluoro-4-(piperazin-1- yl)phenyl)-6-(pyridin-2-yl)- 8,9- dihydroimidazo[1′,2′:1,6] pyrido[2,3-d]pyrimidin-2-amine |
| 33 | 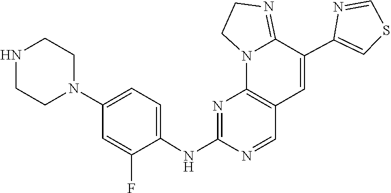 [SMILES: Fc1cc(N2CCNCC2)ccc1Nc1ncc2c(n1)N1CCN=C1C(c1cscn1)=C2] | N-(2-fluoro-4-(piperazin-1- yl)phenyl)-6-(thiazol-4-yl)- 8,9- dihydroimidazo[1′,2′:1,6] pyrido[2,3-d]pyrimidin-2-amine |
| 34 | 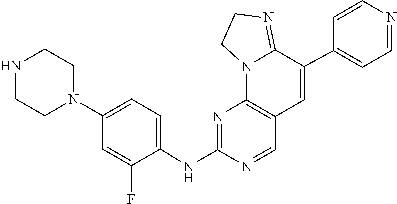 [SMILES: Fc1cc(N2CCNCC2)ccc1Nc1ncc2c(n1)N1CCN=C1C(c1ccncc1)=C2] | N-(2-fluoro-4-(piperazin-1- yl)phenyl)-6-(pyridin-4-yl)- 8,9- dihydroimidazo[1′,2′:1,6] pyrido[2,3-d]pyrimidin-2-amine |
| 35 | 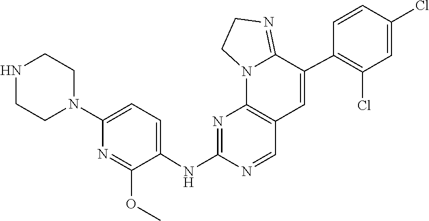 [SMILES: COc1nc(N2CCNCC2)ccc1Nc1ncc2c(n1)N1CCN=C1C(c1ccc(Cl)cc1Cl)=C2] | 6-(2,4-dichlorophenyl)-N-(2- methoxy-6-(piperazin-1- yl)pyridin-3-yl)-8,9- dihydroimidazo[1′,2′:1,6] pyrido[2,3-d]pyrimidin-2-amine |
| 36 | 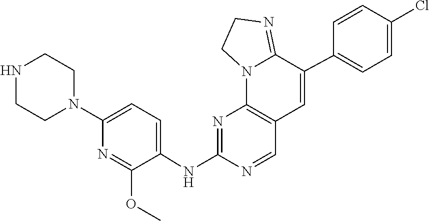 [SMILES: COc1nc(N2CCNCC2)ccc1Nc1ncc2c(n1)N1CCN=C1C(c1ccc(Cl)cc1)=C2] | 6-(4-chlorophenyl)-N-(2- methoxy-6-(piperazin-1- yl)pyridin-3-yl)-8,9- dihydroimidazo[1′,2′:1,6] pyrido[2,3-d]pyrimidin-2-amine |
| 37 | 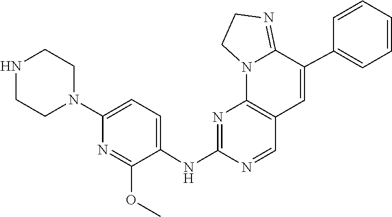 [SMILES: COc1nc(N2CCNCC2)ccc1Nc1ncc2c(n1)N1CCN=C1C(c1ccccc1)=C2] | N-(2-methoxy-6-(piperazin-1- yl)pyridin-3-yl)-6-phenyl-8,9- dihydroimidazo[1′,2′:1,6] pyrido[2,3-d]pyrimidin-2-amine |
| 38 | 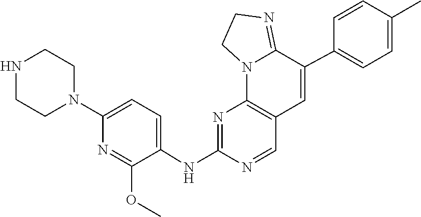 [SMILES: COc1nc(N2CCNCC2)ccc1Nc1ncc2c(n1)N1CCN=C1C(c1ccc(C)cc1)=C2] | N-(2-methoxy-6-(piperazin-1- yl)pyridin-3-yl)-6-(p-tolyl)- 8,9- dihydroimidazo[1′,2′:1,6] pyrido[2,3-d]pyrimidin-2-amine |
| 39 | 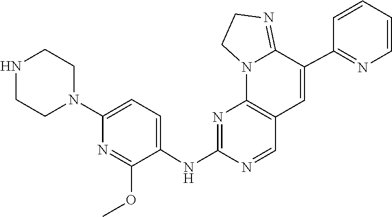 [SMILES: COc1nc(N2CCNCC2)ccc1Nc1ncc2c(n1)N1CCN=C1C(c1ccccn1)=C2] | N-(2-methoxy-6-(piperazin-1- yl)pyridin-3-yl)-6-(pyridin-2- yl)-8,9- dihydroimidazo[1′,2′:1,6] pyrido[2,3-d]pyrimidin-2-amine |
| 40 | 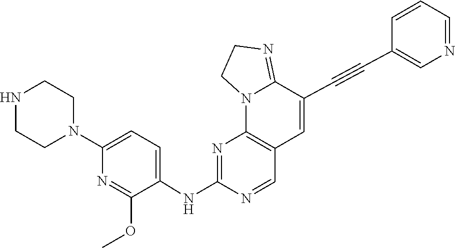 [SMILES: COc1nc(N2CCNCC2)ccc1Nc1ncc2c(n1)N1CCN=C1C(C#Cc1cccnc1)=C2] | N-(2-methoxy-6-(piperazin-1- yl)pyridin-3-yl)-6-(pyridin-3- ylethynyl)-8,9- dihydroimidazo[1′,2′:1,6] pyrido[2,3-d]pyrimidin-2-amine |
| 41 | 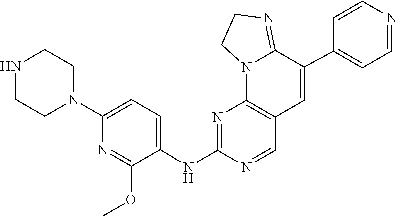 [SMILES: COc1nc(N2CCNCC2)ccc1Nc1ncc2c(n1)N1CCN=C1C(c1ccncc1)=C2] | N-(2-methoxy-6-(piperazin-1- yl)pyridin-3-yl)-6-(pyridin-4- yl)-8,9- dihydroimidazo[1′,2′:1,6] pyrido[2,3-d]pyrimidin-2-amine |
| 42 | 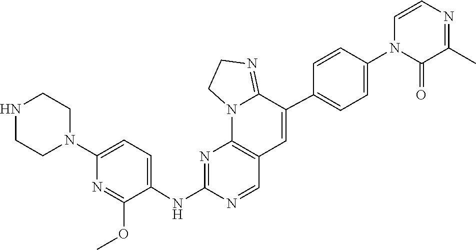 [SMILES: COc1nc(N2CCNCC2)ccc1Nc1ncc2c(n1)N1CCN=C1C(c1ccc(-n3ccnc(C)c3=O)cc1)=C2] | 1-(4-(2-((2-methoxy-6- (piperazin-1-yl)pyridin-3- yl)amino)-8,9- dihydroimidazo[1′,2′:1,6] pyrido[2,3-d]pyrimidin-6- yl)phenyl)-3-methylpyrazin- 2(1H)-one |
| 43 | 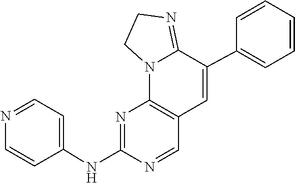 [SMILES: C1=C(c2ccccc2)C2=NCCN2c2nc(Nc3ccncc3)ncc21] | 6-phenyl-N-(pyridin-4-yl)- 8,9- dihydroimidazo[1′,2′:1,6] pyrido[2,3-d]pyrimidin-2-amine |
| 44 | 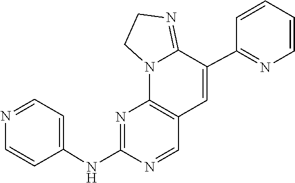 [SMILES: C1=C(c2ccccn2)C2=NCCN2c2nc(Nc3ccncc3)ncc21] | 6-(pyridin-2-yl)-N-(pyridin-4- yl)-8,9- dihydroimidazo[1′,2′:1,6] pyrido[2,3-d]pyrimidin-2-amine |
| 45 | 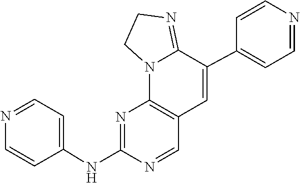 [SMILES: C1=C(c2ccncc2)C2=NCCN2c2nc(Nc3ccncc3)ncc21] | N,6-di(pyridin-4-yl)-8,9- dihydroimidazo[1′,2′:1,6] pyrido[2,3-d]pyrimidin-2-amine |
| 46 | 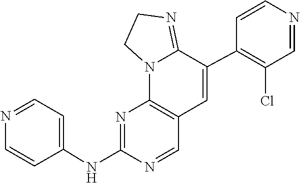 [SMILES: Clc1cnccc1C1=Cc2cnc(Nc3ccncc3)nc2N2CCN=C12] | 6-(3-chloropyridin-4-yl)-N- (pyridin-4-yl)-8,9- dihydroimidazo[1′,2′:1,6] pyrido[2,3-d]pyrimidin-2-amine |
| 47 | 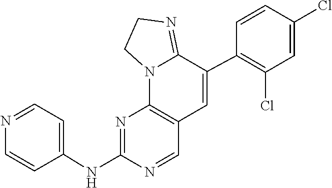 [SMILES: Clc1ccc(C2=Cc3cnc(Nc4ccncc4)nc3N3CCN=C23)c(Cl)c1] | 6-(2,4-dichlorophenyl)-N- (pyridin-4-yl)-8,9- dihydroimidazo[1′,2′:1,6] pyrido[2,3-d]pyrimidin-2-amine |
| 48 | 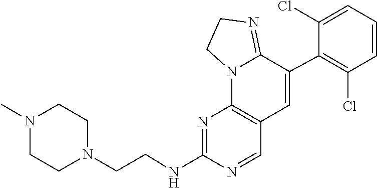 [SMILES: CN1CCN(CCNc2ncc3c(n2)N2CCN=C2C(c2c(Cl)cccc2Cl)=C3)CC1] | 6-(2,6-dichlorophenyl)-N-(2- (4-methylpiperazin-1- yl)ethyl)-8,9- dihydroimidazo[1′,2′:1,6] pyrido[2,3-d]pyrimidin-2-amine |
| 49 | 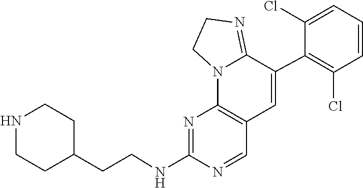 [SMILES: Clc1cccc(Cl)c1C1=Cc2cnc(NCCC3CCNCC3)nc2N2CCN=C12] | 6-(2,6-dichlorophenyl)-N-(2- (piperidin-4-yl)ethyl)-8,9- dihydroimidazo[1′,2′:1,6] pyrido[2,3-d]pyrimidin-2-amine |
| 50 | 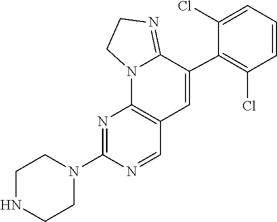 [SMILES: Clc1cccc(Cl)c1C1=Cc2cnc(N3CCNCC3)nc2N2CCN=C12] | 6-(2,6-dichlorophenyl)-2- (piperazin-1-yl)-8,9- dihydroimidazo[1′,2′:1,6] pyrido[2,3-d]pyrimidine |
| 51 | 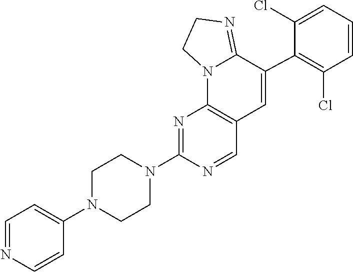 [SMILES: Clc1cccc(Cl)c1C1=Cc2cnc(N3CCN(c4ccncc4)CC3)nc2N2CCN=C12] | 6-(2,6-dichlorophenyl)-2-(4- (pyridin-4-yl)piperazin-1-yl)- 8,9- dihydroimidazo[1′,2′:1,6] pyrido[2,3-d]pyrimidine |
| 52 | 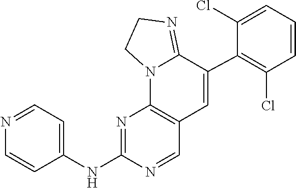 [SMILES: Clc1cccc(Cl)c1C1=Cc2cnc(Nc3ccncc3)nc2N2CCN=C12] | 6-(2,6-dichlorophenyl)-N- (pyridin-4-yl)-8,9- dihydroimidazo[1′,2′:1,6] pyrido[2,3-d]pyrimidin-2-amine |
| 53 | 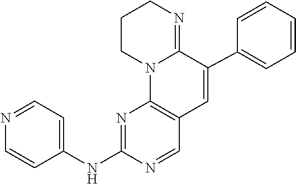 [SMILES: C1=C(c2ccccc2)C2=NCCCN2c2nc(Nc3ccncc3)ncc21] | 6-phenyl-N-(pyridin-4-yl)- 9,10-dihydro-8H-pyrido[1,6- a:2,3-d′]dipyrimidin-2-amine |
| 54 | 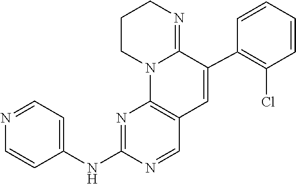 [SMILES: Clc1ccccc1C1=Cc2cnc(Nc3ccncc3)nc2N2CCCN=C12] | 6-(2-chlorophenyl)-N- (pyridin-4-y1)-9,10-dihydro- 8H-pyrido[1,6-a:2,3- d′]dipyrimidin-2-amine |
| 55 | 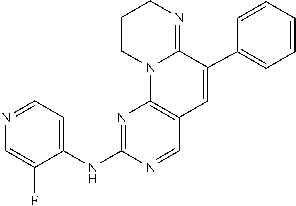 [SMILES: Fc1cnccc1Nc1ncc2c(n1)N1CCCN=C1C(c1ccccc1)=C2] | N-(3-fluoropyridin-4-yl)-6- phenyl-9,10-dihydro-8H- pyrido[1,6-a:2,3- d′]dipyrimidin-2-amine |
| 56 | 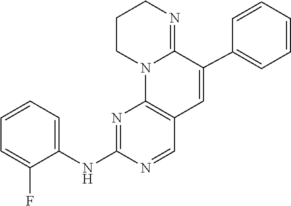 [SMILES: Fc1ccccc1Nc1ncc2c(n1)N1CCCN=C1C(c1ccccc1)=C2] | N-(2-fluorophenyl)-6-phenyl- 9,10-dihydro-8H-pyrido[1,6- a:2,3-d′]dipyrimidin-2-amine |
| 57 | 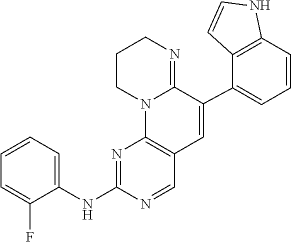 [SMILES: Fc1ccccc1Nc1ncc2c(n1)N1CCCN=C1C(c1cccc3[nH]ccc13)=C2] | N-(2-fluorophenyl)-6-(1H- indol-4-yl)-9,10-dihydro-8H- pyrido[1,6-a:2,3- d′]dipyrimidin-2-amine |
| 58 | 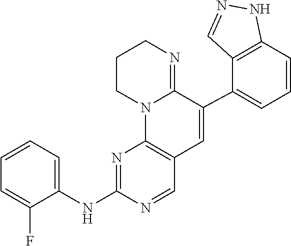 [SMILES: Fc1ccccc1Nc1ncc2c(n1)N1CCCN=C1C(c1cccc3[nH]ncc13)=C2] | N-(2-fluorophenyl)-6-(1H- indazol-4-yl)-9,10-dihydro- 8H-pyrido[1,6-a:2,3- d′]dipyrimidin-2-amine |
| 59 | 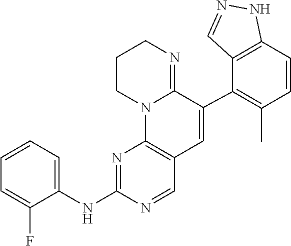 [SMILES: Cc1ccc2[nH]ncc2c1C1=Cc2cnc(Nc3ccccc3F)nc2N2CCCN=C12] | N-(2-fluorophenyl)-6-(5- methyl-1H-indazol-4-yl)- 9,10-dihydro-8H-pyrido[1,6- a:2,3-d′]dipyrimidin-2-amine |
| 60 | 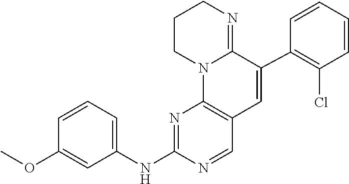 [SMILES: COc1cccc(Nc2ncc3c(n2)N2CCCN=C2C(c2ccccc2Cl)=C3)c1] | 6-(2-chlorophenyl)-N-(3- methoxyphenyl)-9,10- dihydro-8H-pyrido[1,6-a:2,3- d′]dipyrimidin-2-amine |
| 61 | 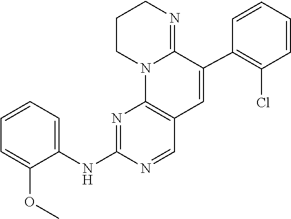 [SMILES: COc1ccccc1Nc1ncc2c(n1)N1CCCN=C1C(c1ccccc1Cl)=C2] | 6-(2-chlorophenyl)-N-(2- methoxyphenyl)-9,10- dihydro-8H-pyrido[1,6-a:2,3- d′]dipyrimidin-2-amine |
| 62 | 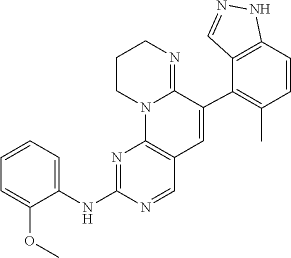 [SMILES: COc1ccccc1Nc1ncc2c(n1)N1CCCN=C1C(c1c(C)ccc3[nH]ncc13)=C2] | N-(2-methoxyphenyl)-6-(5- methyl-1H-indazol-4-yl)- 9,10-dihydro-8H-pyrido[1,6- a:2,3-d′]dipyrimidin-2-amine |
| 63 | 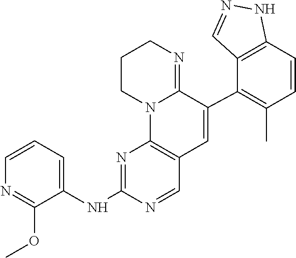 [SMILES: COc1ncccc1Nc1ncc2c(n1)N1CCCN=C1C(c1c(C)ccc3[nH]ncc13)=C2] | N-(2-methoxypyridin-3-yl)-6- (5-methyl-1H-indazol-4-yl)- 9,10-dihydro-8H-pyrido[1,6- a:2,3-d′]dipyrimidin-2-amine |
| 64 | 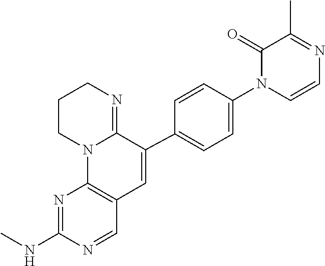 [SMILES: CNc1ncc2c(n1)N1CCCN=C1C(c1ccc(-n3ccnc(C)c3=O)cc1)=C2] | 3-methyl-1-(4-(2- (methylamino)-9,10-dihydro- 8H-pyrido[1,6-a:2,3- d′]dipyrimidin-6- yl)phenyl)pyrazin-2(1H)-one |
| 65 | 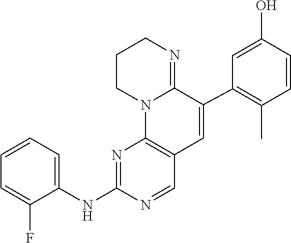 [SMILES: Cc1ccc(O)cc1C1=Cc2cnc(Nc3ccccc3F)nc2N2CCCN=C12] | 3-(2-((2-fluorophenyl)amino)- 9,10-dihydro-8H-pyrido[1,6- a:2,3-d′]dipyrimidin-6-yl)-4- methylphenol |
| 66 | 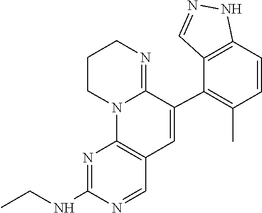 [SMILES: CCNc1ncc2c(n1)N1CCCN=C1C(c1c(C)ccc3[nH]ncc13)=C2] | N-ethyl-6-(5-methyl-1H- indazol-4-yl)-9,10-dihydro- 8H-pyrido[1,6-a:2,3- d′]dipyrimidin-2-amine |
| 67 | 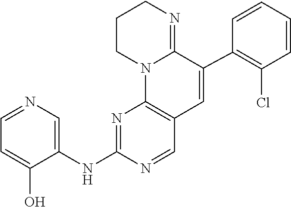 [SMILES: Oc1ccncc1Nc1ncc2c(n1)N1CCCN=C1C(c1ccccc1Cl)=C2] | 3-((6-(2-chlorophenyl)-9,10- dihydro-8H-pyrido[1,6-a:2,3- d′]dipyrimidin-2- yl)amino)pyridin-4-ol |
| 68 | 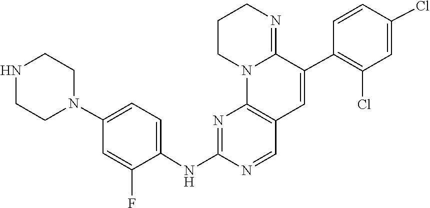 [SMILES: Fc1cc(N2CCNCC2)ccc1Nc1ncc2c(n1)N1CCCN=C1C(c1ccc(Cl)cc1Cl)=C2] | 6-(2,4-dichlorophenyl)-N-(2- fluoro-4-(piperazin-1- yl)phenyl)-9,10-dihydro-8H- pyrido[1,6-a:2,3- d′]dipyrimidin-2-amine |
| 69 | 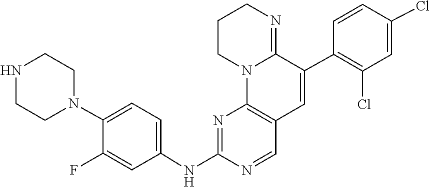 [SMILES: Fc1cc(Nc2ncc3c(n2)N2CCCN=C2C(c2ccc(Cl)cc2Cl)=C3)ccc1N1CCNCC1] | 6-(2,4-dichlorophenyl)-N-(3- fluoro-4-(piperazin-1- yl)phenyl)-9,10-dihydro-8H- pyrido[1,6-a:2,3- d′]dipyrimidin-2-amine |
| 70 | 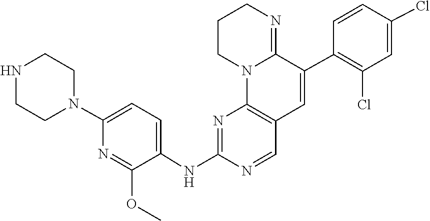 [SMILES: COc1nc(N2CCNCC2)ccc1Nc1ncc2c(n1)N1CCCN=C1C(c1ccc(Cl)cc1Cl)=C2] | 6-(2,4-dichlorophenyl)-N-(2- methoxy-6-(piperazin-1- yl)pyridin-3-yl)-9,10-dihydro- 8H-pyrido[1,6-a:2,3- d′]dipyrimidin-2-amine |
| 71 | 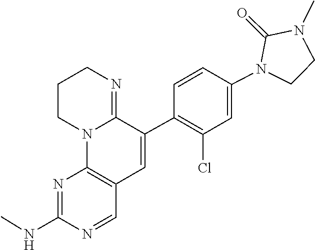 [SMILES: CNc1ncc2c(n1)N1CCCN=C1C(c1ccc(N3CCN(C)C3=O)cc1Cl)=C2] | 1-(3-chloro-4-(2- (methylamino)-9,10-dihydro- 8H-pyrido[1,6-a:2,3- d′]dipyrimidin-6-yl)phenyl)- 3-methylimidazolidin-2-one |
| 72 | 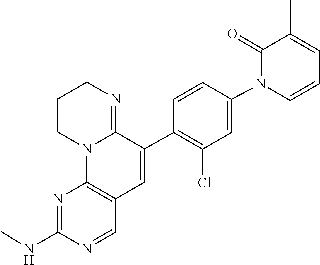 [SMILES: CNc1ncc2c(n1)N1CCCN=C1C(c1ccc(-n3cccc(C)c3=O)cc1Cl)=C2] | 1-(3-chloro-4-(2- (methylamino)-9,10-dihydro- 8H-pyrido[1,6-a:2,3- d′]dipyrimidin-6-yl)phenyl)- 3-methylpyridin-2(1H)-one |
| 73 | 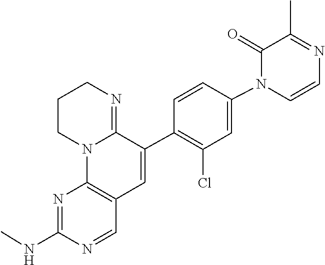 [SMILES: CNc1ncc2c(n1)N1CCCN=C1C(c1ccc(-n3ccnc(C)c3=O)cc1Cl)=C2] | 1-(3-chloro-4-(2- (methylamino)-9,10-dihydro- 8H-pyrido[1,6-a:2,3- d′]dipyrimidin-6-yl)phenyl)- 3-methylpyrazin-2(1H)-one |
| 74 | 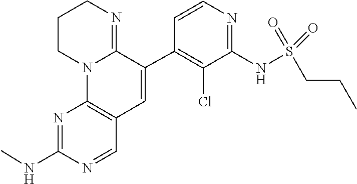 [SMILES: CCCS(=O)(=O)Nc1nccc(C2=Cc3cnc(NC)nc3N3CCCN=C23)c1Cl] | N-(3-chloro-4-(2- (methylamino)-9,10-dihydro- 8H-pyrido[1,6-a:2,3- d′]dipyrimidin-6-yl)pyridin-2- yl)propane-1-sulfonamide |
| 75 | 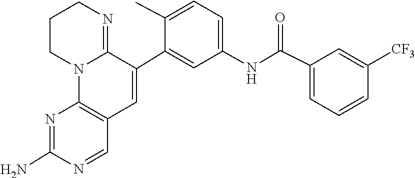 [SMILES: Cc1ccc(NC(=O)c2cccc(C(F)(F)F)c2)cc1C1=Cc2cnc(N)nc2N2CCCN=C12] | N-(3-(2-amino-9,10-dihydro- 8H-pyrido[1,6-a:2,3- d′]dipyrimidin-6-yl)-4- methylphenyl)-3- (trifluoromethyl)benzamide |
| 76 | 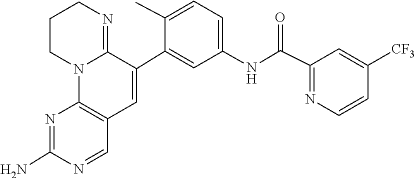 [SMILES: Cc1ccc(NC(=O)c2cc(C(F)(F)F)ccn2)cc1C1=Cc2cnc(N)nc2N2CCCN=C12] | N-(3-(2-amino-9,10-dihydro- 8H-pyrido[1,6-a:2,3- d′]dipyrimidin-6-yl)-4- methylphenyl)-4- (trifluoromethyl)picolinamide |
| 77 | 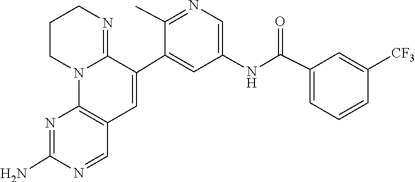 [SMILES: Cc1ncc(NC(=O)c2cccc(C(F)(F)F)c2)cc1C1=Cc2cnc(N)nc2N2CCCN=C12] | N-(5-(2-amino-9,10-dihydro- 8H-pyrido[1,6-a:2,3- d′]dipyrimidin-6-yl)-6- methylpyridin-3-yl)-3- (trifluoromethyl)benzamide |
| 78 | 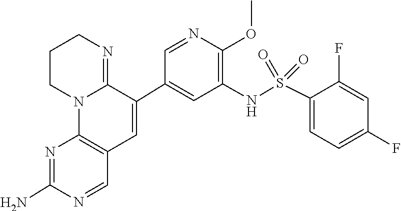 [SMILES: COc1ncc(C2=Cc3cnc(N)nc3N3CCCN=C23)cc1NS(=O)(=O)c1ccc(F)cc1F] | N-(5-(2-amino-9,10-dihydro- 8H-pyrido[1,6-a:2,3- d′]dipyrimidin-6-yl)-2- methoxypyridin-3-yl)-2,4- difluorobenzenesulfonamide |
| 79 | 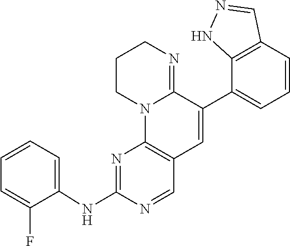 [SMILES: Fc1ccccc1Nc1ncc2c(n1)N1CCCN=C1C(c1cccc3cn[nH]c13)=C2] | N-(2-fluorophenyl)-6-(1H- indazol-7-y1)-9,10-dihydro- 8H-pyrido[1,6-a:2,3- d′]dipyrimidin-2-amine |
| 80 | 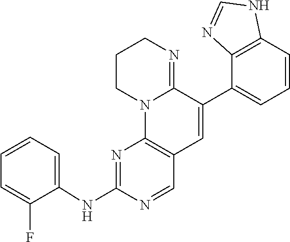 [SMILES: Fc1ccccc1Nc1ncc2c(n1)N1CCCN=C1C(c1cccc3[nH]cnc13)=C2] | 6-(1H-benzo[d]imidazol-4- yl)-N-(2-fluorophenyl)-9,10- dihydro-8H-pyrido[1,6-a:2,3- d′]dipyrimidin-2-amine |
| 81 | 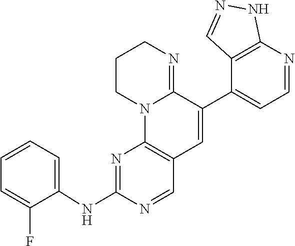 [SMILES: Fc1ccccc1Nc1ncc2c(n1)N1CCCN=C1C(c1ccnc3[nH]ncc13)=C2] | N-(2-fluorophenyl)-6-(1H- pyrazolo[3,4-b]pyridin-4-yl)- 9,10-dihydro-8H-pyrido[1,6- a:2,3-d′]dipyrimidin-2-amine |

and pharmaceutically acceptable salts thereof.

In some embodiments, provided herein are compounds and salts thereof described in Table 2, and uses thereof.

TABLE 2

| Cpd | | |
|---|---|---|
| No. | Structure | Chemical Name |
| 82 | 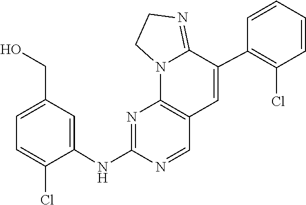 [SMILES: OCc1ccc(Cl)c(Nc2ncc3c(n2)N2CCN=C2C(c2ccccc2Cl)=C3)c1] | (4-chloro-3-((6-(2- chlorophenyl)-8,9- dihydroimidazo[1′2′:1,6] pyrido[2,3-d]pyrimidin- 2- yl)amino)phenyl) methanol |
| 83 | 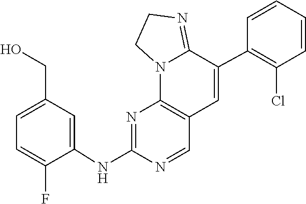 [SMILES: OCc1ccc(F)c(Nc2ncc3c(n2)N2CCN=C2C(c2ccccc2Cl)=C3)c1] | (3-((6-(2-chlorophenyl)- 8,9- dihydroimidazo[1′2′:1,6] pyrido[2,3-d]pyrimidin- 2-yl)amino)-4- fluorophenyl)methanol |
| 84 | 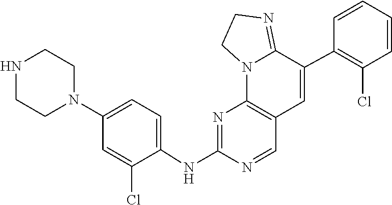 [SMILES: Clc1cc(N2CCNCC2)ccc1Nc1ncc2c(n1)N1CCN=C1C(c1ccccc1Cl)=C2] | N-(2-chloro-4- (piperazin-1-yl)phenyl)- 6-(2-chlorophenyl)-8,9- dihydroimidazo[1′2′:1,6] pyrido[2,3-d]pyrimidin- 2-amine |
| 85 | 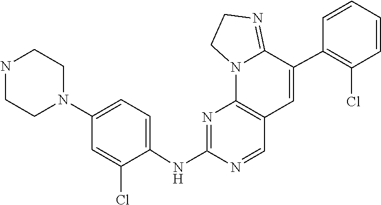 [SMILES: Clc1cc(N2CCNCC2)ccc1Nc1ncc2c(n1)N1CCN=C1C(c1ccccc1Cl)=C2] | N-(2-chloro-4-(4- methylpiperazin-1- yl)phenyl)-6-(2- chlorophenyl)-8,9- dihydroimidazo[1′2′:1,6] pyrido[2,3-d]pyrimidin- 2-amine |
| 86 | 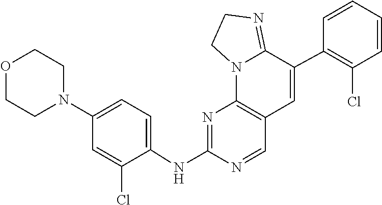 [SMILES: Clc1cc(N2CCOCC2)ccc1Nc1ncc2c(n1)N1CCN=C1C(c1ccccc1Cl)=C2] | N-(2-chloro-4- morpholinophenyl)-6-(2- chlorophenyl)-8,9- dihydroimidazo[1′2′:1,6] pyrido[2,3-d]pyrimidin- 2-amine |
| 87 | 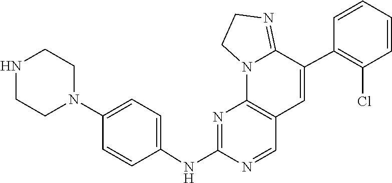 [SMILES: Clc1ccccc1C1=Cc2cnc(Nc3ccc(N4CCNCC4)cc3)nc2N2CCN=C12] | 6-(2-chlorophenyl)-N-(4- (piperazin-1-yl)phenyl)- 8,9- dihydroimidazo[1′2′:1,6] pyrido[2,3-d]pyrimidin- 2-amine |
| 88 | 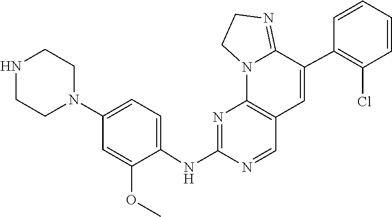 [SMILES: COc1cc(N2CCNCC2)ccc1Nc1ncc2c(n1)N1CCN=C1C(c1ccccc1Cl)=C2] | 6-(2-chlorophenyl)-N-(2- methoxy-4-(piperazin-1- yl)phenyl)-8,9- dihydroimidazo[1′2′:1,6] pyrido[2,3-d]pyrimidin- 2-amine |
| 89 | 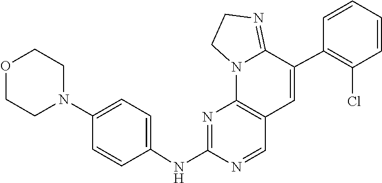 [SMILES: Clc1ccccc1C1=Cc2cnc(Nc3ccc(N4CCOCC4)cc3)nc2N2CCN=C12] | 6-(2-chlorophenyl)-N-(4- morpholinophenyl)-8,9- dihydroimidazo[1′2′:1,6] pyrido[2,3-d]pyrimidin- 2-amine |
| 90 | 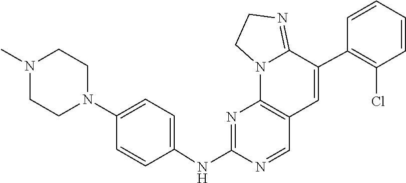 [SMILES: CN1CCN(c2ccc(Nc3ncc4c(n3)N3CCN=C3C(c3ccccc3Cl)=C4)cc2)CC1] | 6-(2-chlorophenyl)-N-(4- (4-methylpiperazin-1- yl)phenyl)-8,9- dihydroimidazo[1′2′:1,6] pyrido[2,3-d]pyrimidin- 2-amine |
| 91 | 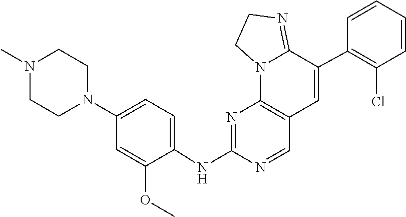 [SMILES: COc1cc(N2CCN(C)CC2)ccc1Nc1ncc2c(n1)N1CCN=C1C(c1ccccc1Cl)=C2] | 6-(2-chlorophenyl)-N-(2- methoxy-4-(4- methylpiperazin-1- yl)phenyl)-8,9- dihydroimidazo[1′2′:1,6] pyrido[2,3-d]pyrimidin- 2-amine |
| 92 | 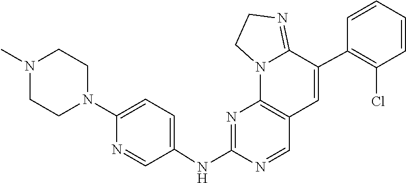 [SMILES: CN1CCN(c2ccc(Nc3ncc4c(n3)N3CCN=C3C(c3ccccc3Cl)=C4)cn2)CC1] | 6-(2-chlorophenyl)-N-(6- (4-methylpiperazin-1- yl)pyridin-3-yl)-8,9- dihydroimidazo[1′2′:1,6] pyrido[2,3-d]pyrimidin- 2-amine |
| 93 | 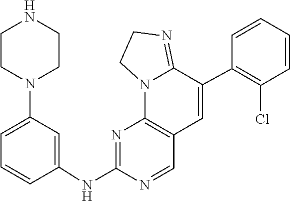 [SMILES: Clc1ccccc1C1=Cc2cnc(Nc3cccc(N4CCNCC4)c3)nc2N2CCN=C12] | 6-(2-chlorophenyl)-N-(3- (piperazin-1-yl)phenyl)- 8,9- dihydroimidazo[1′2′:1,6] pyrido[2,3-d]pyrimidin- 2-amine |
| 94 | 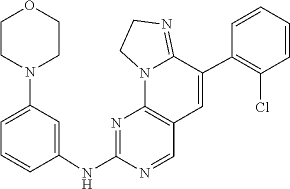 [SMILES: Clc1ccccc1C1=Cc2cnc(Nc3cccc(N4CCOCC4)c3)nc2N2CCN=C12] | 6-(2-chlorophenyl)-N-(3- morpholinophenyl)-8,9- dihydroimidazo[1′2′:1,6] pyrido[2,3-d]pyrimidin- 2-amine |
| 95 | 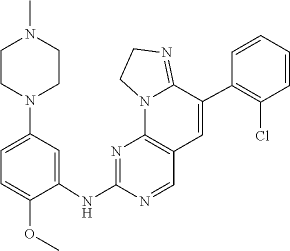 [SMILES: COc1ccc(N2CCN(C)CC2)cc1Nc1ncc2c(n1)N1CCN=C1C(c1ccccc1Cl)=C2] | 6-(2-chlorophenyl)-N-(2- methoxy-5-(4- methylpiperazin-1- yl)phenyl)-8,9- dihydroimidazo[1′2′:1,6] pyrido[2,3-d]pyrimidin- 2-amine |
| 96 | 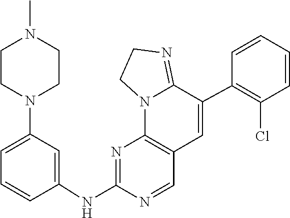 [SMILES: CN1CCN(c2cccc(Nc3ncc4c(n3)N3CCN=C3C(c3ccccc3Cl)=C4)c2)CC1] | 6-(2-chlorophenyl)-N-(3- (4-methylpiperazin-1- yl)phenyl)-8,9- dihydroimidazo[1′2′:1,6] pyrido[2,3-d]pyrimidin- 2-amine |
| 97 | 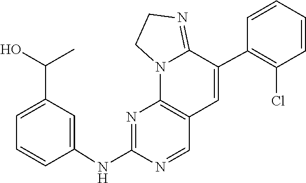 [SMILES: CC(O)c1cccc(Nc2ncc3c(n2)N2CCN=C2C(c2ccccc2Cl)=C3)c1] | 1-(3-((6-(2- chlorophenyl)-8,9- dihydroimidazo[1′2′:1,6] pyrido[2,3-d]pyrimidin- 2- yl)amino)phenyl)ethan- 1-o1 |
| 98 | 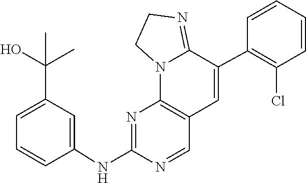 [SMILES: CC(C)(O)c1cccc(Nc2ncc3c(n2)N2CCN=C2C(c2ccccc2Cl)=C3)c1] | 2-(3-((6-(2- chlorophenyl)-8,9- dihydroimidazo[1′2′:1,6] pyrido[2,3-d]pyrimidin- 2- yl)amino)phenyl)propan- 2-ol |
| 99 | 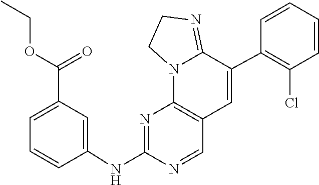 [SMILES: CCOC(=O)c1cccc(Nc2ncc3c(n2)N2CCN=C2C(c2ccccc2Cl)=C3)c1] | ethyl 3-((6-(2- chloropheny1)-8,9- dihydroimidazo[1′2′:1,6] pyrido[2,3-d]pyrimidin- 2-yl)amino)benzoate |
| 100 | 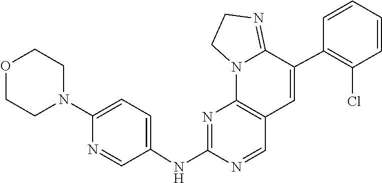 [SMILES: Clc1ccccc1C1=Cc2cnc(Nc3ccc(N4CCOCC4)nc3)nc2N2CCN=C12] | 6-(2-chlorophenyl)-N-(6- morpholinopyridin-3-yl)- 8,9- dihydroimidazo[1′2′:1,6] pyrido[2,3-d]pyrimidin- 2-amine |
| 101 | 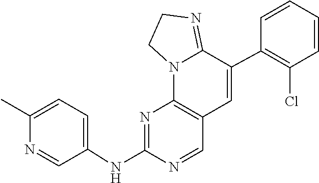 [SMILES: Cc1ccc(Nc2ncc3c(n2)N2CCN=C2C(c2ccccc2Cl)=C3)cn1] | 6-(2-chlorophenyl)-N-(6- methylpyridin-3-yl)-8,9- dihydroimidazo[1′2′:1,6] pyrido[2,3-d]pyrimidin- 2-amine |
| 102 | 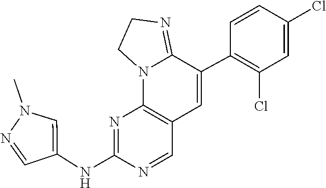 [SMILES: Cn1cc(Nc2ncc3c(n2)N2CCN=C2C(c2ccc(Cl)cc2Cl)=C3)cn1] | 6-(2,4-dichlorophenyl)- N-(1-methyl-1H-pyrazol- 4-yl)-8,9- dihydroimidazo[1′2′:1,6] pyrido[2,3-d]pyrimidin- 2-amine |
| 103 | 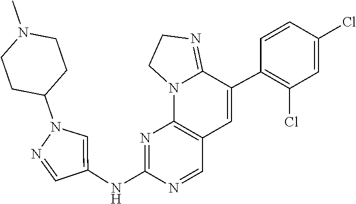 [SMILES: CN1CCC(n2cc(Nc3ncc4c(n3)N3CCN=C3C(c3ccc(Cl)cc3Cl)=C4)cn2)CC1] | 6-(2,4-dichlorophenyl)- N-(1-(1-methylpiperidin- 4-yl)-1H-pyrazol-4-yl)- 8,9- dihydroimidazo[1′2′:1,6] pyrido[2,3-d]pyrimidin- 2-amine |
| 104 | 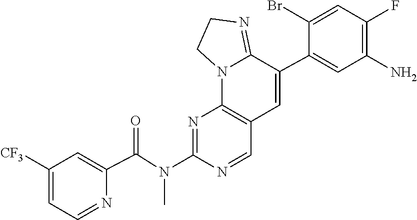 [SMILES: CN(C(=O)c1cc(C(F)(F)F)ccn1)c1ncc2c(n1)N1CCN=C1C(c1cc(N)c(F)cc1Br)=C2] | N-(6-(5-amino-2-bromo- 4-fluorophenyl)-8,9- dihydroimidazo[1′2′:1,6] pyrido[2,3-d]pyrimidin- 2-yl)-N-methyl-4- (trifluoromethyl) picolinamide |
| 105 | 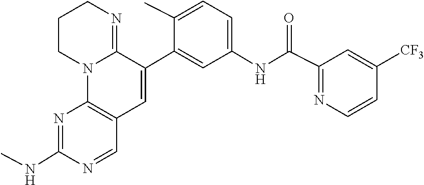 [SMILES: CNc1ncc2c(n1)N1CCCN=C1C(c1cc(NC(=O)c3cc(C(F)(F)F)ccn3)ccc1C)=C2] | N-(4-methyl-3-(2- (methylamino)-9,10- dihydro-8H-pyrido[1,6- a:2,3-d′]dipyrimidin-6- yl)phenyl)-4- (trifluoromethyl) picolinamide |
| 106 | 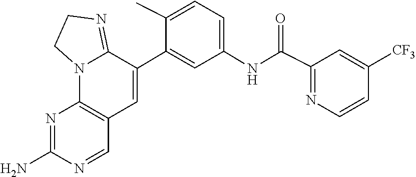 [SMILES: Cc1ccc(NC(=O)c2cc(C(F)(F)F)ccn2)cc1C1=Cc2cnc(N)nc2N2CCN=C12] | N-(3-(2-amino-8,9- dihydroimidazo[1′2′:1,6] pyrido[2,3-d]pyrimidin- 6-yl)-4-methylphenyl)-4- (trifluoromethyl) picolinamide |
| 107 | 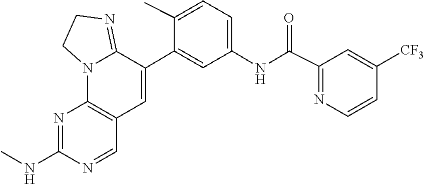 [SMILES: CNc1ncc2c(n1)N1CCN=C1C(c1cc(NC(=O)c3cc(C(F)(F)F)ccn3)ccc1C)=C2] | N-(4-methyl-3-(2- (methylamino)-8,9- dihydroimidazo[1′2′:1,6] pyrido[2,3-d]pyrimidin- 6-yl)phenyl)-4- (trifluoromethyl) picolinamide |
| 108 | 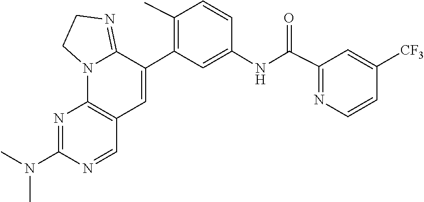 [SMILES: Cc1ccc(NC(=O)c2cc(C(F)(F)F)ccn2)cc1C1=Cc2cnc(N(C)C)nc2N2CCN=C12] | N-(3-(2- (dimethylamino)-8,9- dihydroimidazo[1′2′:1,6] pyrido[2,3-d]pyrimidin- 6-yl)-4-methylphenyl)-4- (trifluoromethyl) picolinamide |
| 109 | 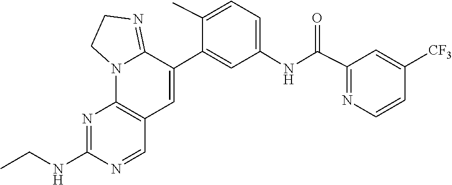 [SMILES: CCNc1ncc2c(n1)N1CCN=C1C(c1cc(NC(=O)c3cc(C(F)(F)F)ccn3)ccc1C)=C2] | N-(3-(2-(ethylamino)- 8,9- dihydroimidazo[1′2′:1,6] pyrido[2,3-d]pyrimidin- 6-yl)-4-methylphenyl)-4- (trifluoromethyl) picolinamide |
| 111 | 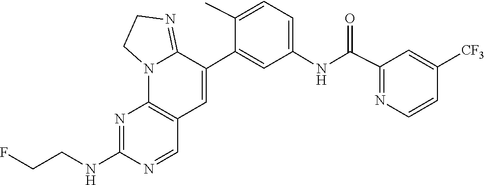 [SMILES: Cc1ccc(NC(=O)c2cc(C(F)(F)F)ccn2)cc1C1=Cc2cnc(NCCF)nc2N2CCN=C12] | N-(3-(2-((2- fluoroethyl)amino)-8,9- dihydroimidazo[1′2′:1,6] pyrido[2,3-d]pyrimidin- 6-yl)-4-methylphenyl)-4- (trifluoromethyl) picolinamide |
| 112 | 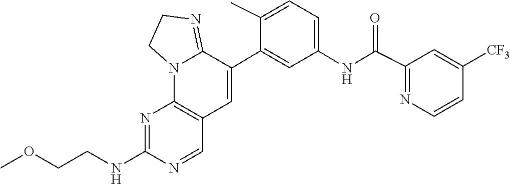 [SMILES: COCCNc1ncc2c(n1)N1CCN=C1C(c1cc(NC(=O)c3cc(C(F)(F)F)ccn3)ccc1C)=C2] | N-(3-(2-((2- methoxyethyl)amino)- 8,9- dihydroimidazo[1′2′:1,6] pyrido[2,3-d]pyrimidin- 6-yl)-4-methylphenyl)-4- (trifluoromethyl) picolinamide |
| 113 | 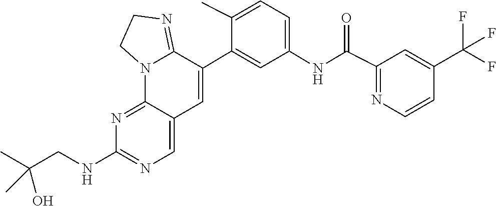 [SMILES: Cc1ccc(NC(=O)c2cc(C(F)(F)F)ccn2)cc1C1=Cc2cnc(NCC(C)(C)O)nc2N2CCN=C12] | N-(3-(2-((2-hydroxy-2- methylpropyl)amino)- 8,9- dihydroimidazo[1′2′:1,6] pyrido[2,3-d]pyrimidin- 6-yl)-4-methylphenyl)-4- (trifluoromethyl) picolinamide |
| 114 | 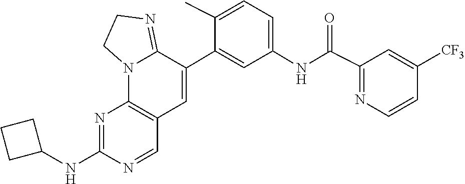 [SMILES: Cc1ccc(NC(=O)c2cc(C(F)(F)F)ccn2)cc1C1=Cc2cnc(NC3CCC3)nc2N2CCN=C12] | N-(4-methyl-3-(2- (oxetan-3-ylamino)-8,9- dihydroimidazo[1′2′:1,6] pyrido[2,3-d]pyrimidin- 6-yl)phenyl)-4- (trifluoromethyl) picolinamide |
| 115 | 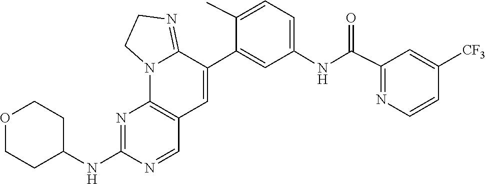 [SMILES: Cc1ccc(NC(=O)c2cc(C(F)(F)F)ccn2)cc1C1=Cc2cnc(NC3CCOCC3)nc2N2CCN=C12] | N-(4-methyl-3-(2- ((tetrahydro-2H-pyran-4- yl)amino)-8,9- dihydroimidazo[1′2′:1,6] pyrido[2,3-d]pyrimidin- 6-yl)phenyl)-4- (trifluoromethyl) picolinamide |
| 116 | 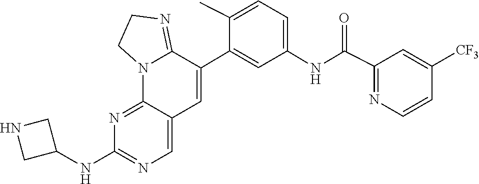 [SMILES: Cc1ccc(NC(=O)c2cc(C(F)(F)F)ccn2)cc1C1=Cc2cnc(NC3CNC3)nc2N2CCN=C12] | N-(3-(2-(azetidin-3- ylamino)-8,9- dihydroimidazo[1′2′:1,6] pyrido[2,3-d]pyrimidin- 6-yl)-4-methylphenyl)-4- (trifluoromethyl) picolinamide |
| 117 | 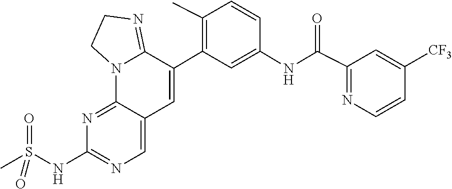 [SMILES: Cc1ccc(NC(=O)c2cc(C(F)(F)F)ccn2)cc1C1=Cc2cnc(NS(C)(=O)=O)nc2N2CCN=C12] | N-(4-methyl-3-(2- (methylsulfonamido)- 8,9- dihydroimidazo[1′2′:1,6] pyrido[2,3-d]pyrimidin- 6-yl)phenyl)-4- (trifluoromethyl) picolinamide |
| 118 | 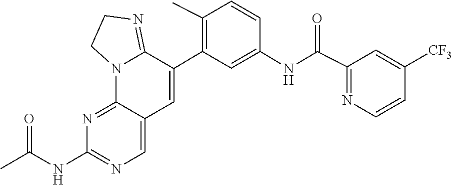 [SMILES: CC(=O)Nc1ncc2c(n1)N1CCN=C1C(c1cc(NC(=O)c3cc(C(F)(F)F)ccn3)ccc1C)=C2] | N-(3-(2-acetamido-8,9- dihydroimidazo[1′2′:1,6] pyrido[2,3-d]pyrimidin- 6-yl)-4-methylphenyl)-4- (trifluoromethyl) picolinamide |
| 119 | 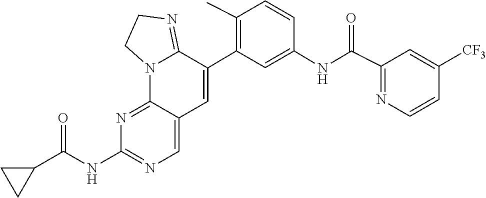 [SMILES: Cc1ccc(NC(=O)c2cc(C(F)(F)F)ccn2)cc1C1=Cc2cnc(NC(=O)C3CC3)nc2N2CCN=C12] | N-(3-(2- (cyclopropane- carboxamido)-8,9- dihydroimidazo[1′2′:1,6] pyrido[2,3-d]pyrimidin- 6-yl)-4-methylphenyl)-4- (trifluoromethyl) picolinamide |
| 120 | 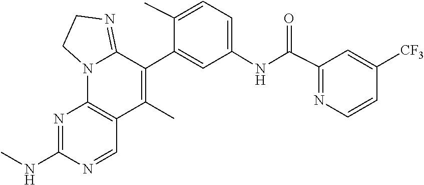 [SMILES: CNc1ncc2c(n1)N1CCN=C1C(c1cc(NC(=O)c3cc(C(F)(F)F)ccn3)ccc1C)=C2C] | N-(4-methyl-3-(5- methyl-2-(methylamino)- 8,9- dihydroimidazo[1′2′:1,6] pyrido[2,3-d]pyrimidin- 6-yl)phenyl)-4- (trifluoromethyl) picolinamide |
| 121 | 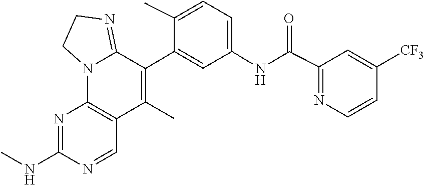 [SMILES: CNc1ncc2c(n1)N1CCN=C1C(c1cc(NC(=O)c3cc(C(F)(F)F)ccn3)ccc1C)=C2C] | N-(4-fluoro-3-(5-methyl- 2-(methylamino)-8,9- dihydroimidazo[1′2′:1,6] pyrido[2,3-d]pyrimidin- 6-yl)phenyl)-4- (trifluoromethyl) picolinamide |
| 122 | 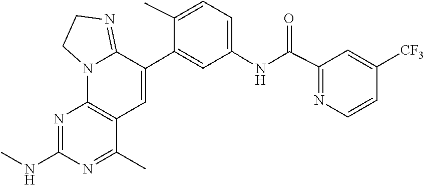 [SMILES: CNc1nc(C)c2c(n1)N1CCN=C1C(c1cc(NC(=O)c3cc(C(F)(F)F)ccn3)ccc1C)=C2] | N-(4-methyl-3-(4- methyl-2-(methylamino)- 8,9- dihydroimidazo[1′2′:1,6] pyrido[2,3-d]pyrimidin- 6-yl)phenyl)-4- (trifluoromethyl) picolinamide |
| 123 | 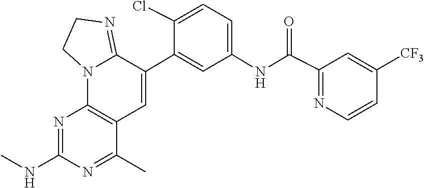 [SMILES: CNc1nc(C)c2c(n1)N1CCN=C1C(c1cc(NC(=O)c3cc(C(F)(F)F)ccn3)ccc1Cl)=C2] | N-(4-chloro-3-(4-methyl- 2-(methylamino)-8,9- dihydroimidazo[1′2′:1,6] pyrido[2,3-d]pyrimidin- 6-yl)phenyl)-4- (trifluoromethyl) picolinamide |
| 124 | 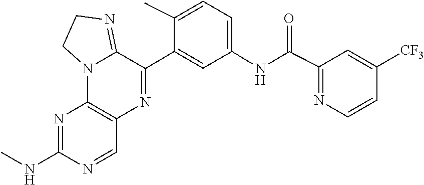 [SMILES: CNc1ncc2c(n1)N1CCN=C1C(c1cc(NC(=O)c3cc(C(F)(F)F)ccn3)ccc1C)=N2] | N-(4-methyl-3-(2- (methylamino)-8,9- dihydroimidazo[2,1- h]pteridin-6-yl)phenyl)- (trifluoromethyl) picolinamide |
| 125 | 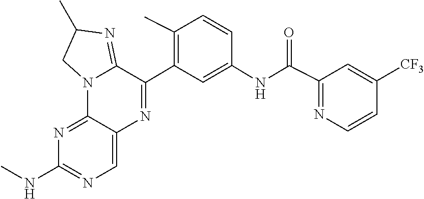 [SMILES: CNc1ncc2c(n1)N1CC(C)N=C1C(c1cc(NC(=O)c3cc(C(F)(F)F)ccn3)ccc1C)=N2] | N-(4-methyl-3-(8- methyl-2-(methylamino)- 8,9- dihydroimidazo[1′2′:1,6] pyrido[2,3-d]pyrimidin- 6-yl)phenyl)-4- (trifluoromethyl) picolinamide |
| 126 | 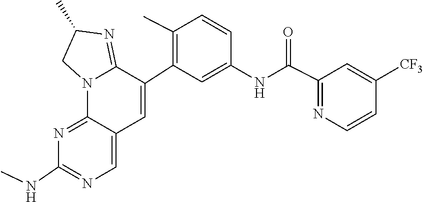 [SMILES: CNc1ncc2c(n1)N1C[C@H](C)N=C1C(c1cc(NC(=O)c3cc(C(F)(F)F)ccn3)ccc1C)=C2] | (S)-N-(4-methyl-3-(8- methyl-2-(methylamino)- 8,9- dihydroimidazo[1′2′:1,6] pyrido[2,3-d]pyrimidin- 6-yl)phenyl)-4- (trifluoromethyl) picolinamide |
| 127 | 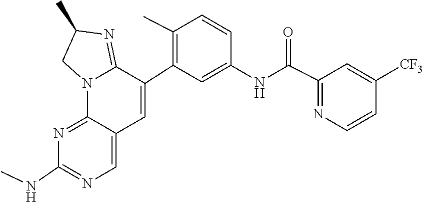 [SMILES: CNc1ncc2c(n1)N1C[C@@H](C)N=C1C(c1cc(NC(=O)c3cc(C(F)(F)F)ccn3)ccc1C)=C2] | (R)-N-(4-methyl-3-(8- methyl-2-(methylamino)- 8,9- dihydroimidazo[1′2′:1,6] pyrido[2,3-d]pyrimidin- 6-yl)phenyl)-4- (trifluoromethyl) picolinamide |
| 128 | 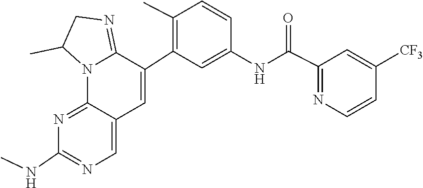 [SMILES: CNc1ncc2c(n1)N1C(=NCC1C)C(c1cc(NC(=O)c3cc(C(F)(F)F)ccn3)ccc1C)=C2] | N-(4-methyl-3-(9- methyl-2-(methylamino)- 8,9- dihydroimidazo[1′2′:1,6] pyrido[2,3-d]pyrimidin- 6-yl)phenyl)-4- (trifluoromethyl) picolinamide |
| 129 | 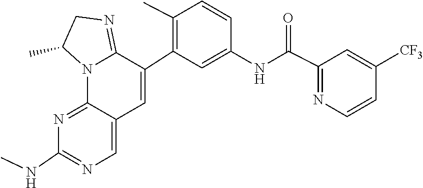 [SMILES: CNc1ncc2c(n1)N1C(=NC[C@H]1C)C(c1cc(NC(=O)c3cc(C(F)(F)F)ccn3)ccc1C)=C2] | (R)-N-(4-methyl-3-(9- methyl-2-(methylamino)- 8,9- dihydroimidazo[1′2′:1,6] pyrido[2,3-d]pyrimidin- 6-yl)phenyl)-4- (trifluoromethyl) picolinamide |
| 130 | 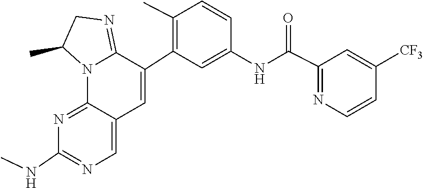 [SMILES: CNc1ncc2c(n1)N1C(=NC[C@@H]1C)C(c1cc(NC(=O)c3cc(C(F)(F)F)ccn3)ccc1C)=C2] | (S)-N-(4-methyl-3-(9- methyl-2-(methylamino)- 8,9- dihydroimidazo[1′2′:1,6] pyrido[2,3-d]pyrimidin- 6-yl)phenyl)-4- (trifluoromethyl) picolinamide |
| 131 | 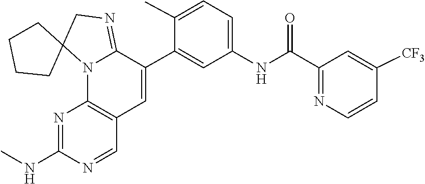 [SMILES: CNc1ncc2c(n1)N1C(=NCC13CCCC3)C(c1cc(NC(=O)c3cc(C(F)(F)F)ccn3)ccc1C)=C2] | N-(4-methyl-3-(2′- (methylamino)-8′H- spiro[cyclopentane-1,9′- imidazo[1′2′:1,6]pyrido [2,3-d]pyrimidin]-6′- yl)phenyl)-4- (trifluoromethyl) picolinamide |
| 132 | 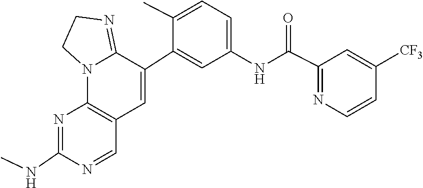 [SMILES: CNc1ncc2c(n1)N1CCN=C1C(c1cc(NC(=O)c3cc(C(F)(F)F)ccn3)ccc1C)=C2] | N-(4-methyl-3-(8- (methylamino)-1,2- dihydroimidazo[1,2- a][1,6]naphthyridin-4- yl)phenyl)-4- (trifluoromethyl) picolinamide |
| 133 | 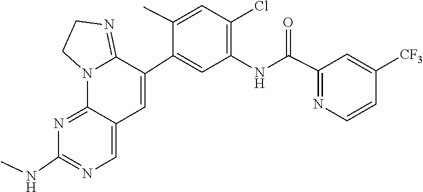 [SMILES: CNc1ncc2c(n1)N1CCN=C1C(c1cc(NC(=O)c3cc(C(F)(F)F)ccn3)c(Cl)cc1C)=C2] | N-(2-chloro-4-methyl-5- (2-(methylamino)-8,9- dihydroimidazo[1′2′:1,6] pyrido[2,3-d]pyrimidin- 6-yl)phenyl)-4- (trifluoromethyl) picolinamide |
| 134 | 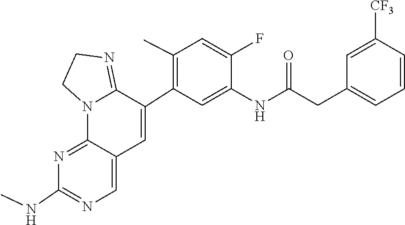 [SMILES: CNc1ncc2c(n1)N1CCN=C1C(c1cc(NC(=O)Cc3cccc(C(F)(F)F)c3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-2-(3- (trifluoromethyl)phenyl) acetamide |
| 135 | 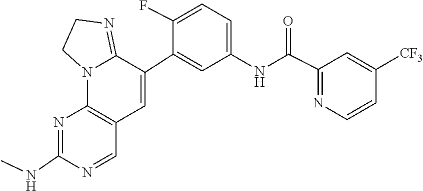 [SMILES: CNc1ncc2c(n1)N1CCN=C1C(c1cc(NC(=O)c3cc(C(F)(F)F)ccn3)ccc1F)=C2] | N-(4-fluoro-3-(2- (methylamino)-8,9- dihydroimidazo[1′2′:1,6] pyrido[2,3-d]pyrimidin- 6-yl)phenyl)-4- (trifluoromethyl) picolinamide |
| 136 | 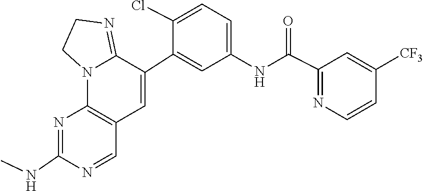 [SMILES: CNc1ncc2c(n1)N1CCN=C1C(c1cc(NC(=O)c3cc(C(F)(F)F)ccn3)ccc1Cl)=C2] | N-(4-chloro-3-(2- (methylamino)-8,9- dihydroimidazo[1′2′:1,6] pyrido[2,3-d]pyrimidin- 6-yl)phenyl)-4- (trifluoromethyl) picolinamide |
| 137 | 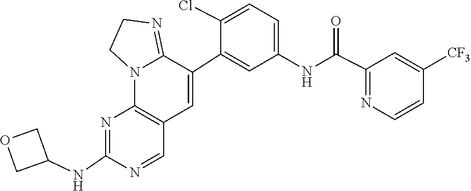 [SMILES: O=C(Nc1ccc(Cl)c(C2=Cc3cnc(NC4COC4)nc3N3CCN=C23)c1)c1cc(C(F)(F)F)ccn1] | N-(4-chloro-3-(2- (oxetan-3-ylamino)-8,9- dihydroimidazo[1′2′:1,6] pyrido[2,3-d]pyrimidin- 6-yl)phenyl)-4- (trifluoromethyl) picolinamide |
| 138 | 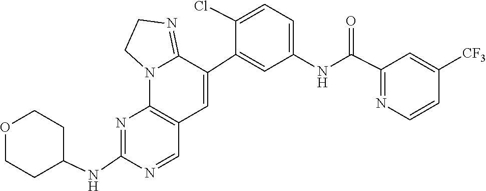 [SMILES: O=C(Nc1ccc(Cl)c(C2=Cc3cnc(NC4CCOCC4)nc3N3CCN=C23)c1)c1cc(C(F)(F)F)ccn1] | N-(4-chloro-3-(2- ((tetrahydro-2H-pyran-4- yl)amino)-8,9- dihydroimidazo[1′2′:1,6] pyrido[2,3-d]pyrimidin- 6-yl)phenyl)-4- (trifluoromethyl) picolinamide |
| 139 | 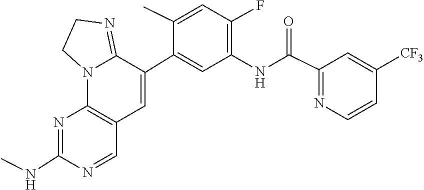 [SMILES: CNc1ncc2c(n1)N1CCN=C1C(c1cc(NC(=O)c3cc(C(F)(F)F)ccn3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-4- (trifluoromethyl) picolinamide |
| 140 | 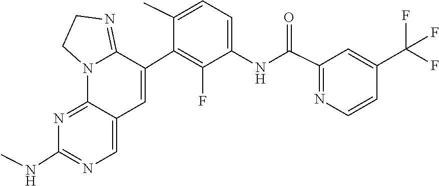 [SMILES: CNc1ncc2c(n1)N1CCN=C1C(c1c(C)ccc(NC(=O)c3cc(C(F)(F)F)ccn3)c1F)=C2] | N-(2-fluoro-4-methyl-3- (2-(methylamino)-8,9- dihydroimidazo[1′2′:1,6] pyrido[2,3-d]pyrimidin- 6-yl)phenyl)-4- (trifluoromethyl) picolinamide |
| 141 | 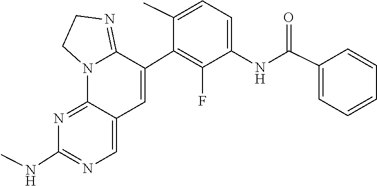 [SMILES: CNc1ncc2c(n1)N1CCN=C1C(c1c(C)ccc(NC(=O)c3ccccc3)c1F)=C2] | N-(2-fluoro-4-methyl-3- (2-(methylamino)-8,9- dihydroimidazo[1′2′:1,6] pyrido[2,3-d]pyrimidin- 6-yl)phenyl)benzamide |
| 142 | 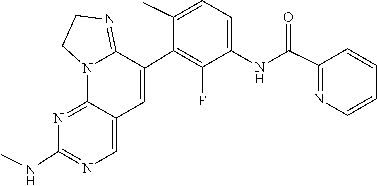 [SMILES: CNc1ncc2c(n1)N1CCN=C1C(c1c(C)ccc(NC(=O)c3ccccn3)c1F)=C2] | N-(2-fluoro-4-methyl-3- (2-(methylamino)-8,9- dihydroimidazo[1′2′:1,6] pyrido[2,3-d]pyrimidin- 6-yl)phenyl)picolinamide |
| 143 | 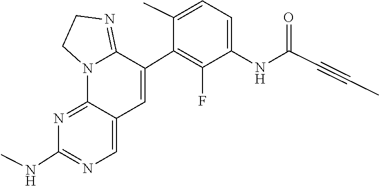 [SMILES: CC#CC(=O)Nc1ccc(C)c(C2=Cc3cnc(NC)nc3N3CCN=C23)c1F] | N-(2-fluoro-4-methyl-3- (2-(methylamino)-8,9- dihydroimidazo[1′2′:1,6] pyrido[2,3-d]pyrimidin- 6-yl)phenyl)but-2- ynamide |
| 144 | 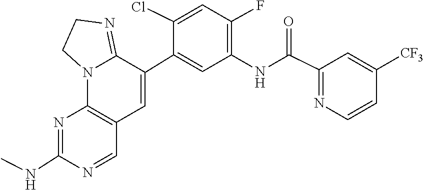 [SMILES: CNc1ncc2c(n1)N1CCN=C1C(c1cc(NC(=O)c3cc(C(F)(F)F)ccn3)c(F)cc1Cl)=C2] | N-(4-chloro-2-fluoro-5- (2-(methylamino)-8,9- dihydroimidazo[1′2′:1,6] pyrido[2,3-d]pyrimidin- 6-yl)phenyl)-4- (trifluoromethyl) picolinamide |
| 145 | 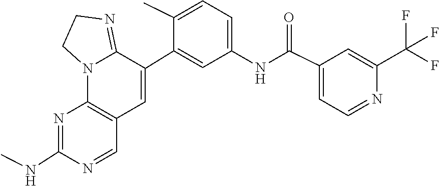 [SMILES: CNc1ncc2c(n1)N1CCN=C1C(c1cc(NC(=O)c3ccnc(C(F)(F)F)c3)ccc1C)=C2] | N-(4-methyl-3-(2- (methylamino)-8,9- dihydroimidazo[1′2′:1,6] pyrido[2,3-d]pyrimidin- 6-yl)phenyl)-2- (trifluoromethyl) isonicotinamide |
| 146 | 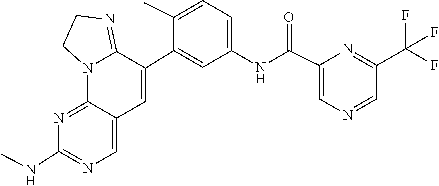 [SMILES: CNc1ncc2c(n1)N1CCN=C1C(c1cc(NC(=O)c3cncc(C(F)(F)F)n3)ccc1C)=C2] | N-(4-methyl-3-(2- (methylamino)-8,9- dihydroimidazo[1′2′:1,6] pyrido[2,3-d]pyrimidin- 6-yl)phenyl)-6- (trifluoromethyl)pyrazine- 2-carboxamide |
| 147 | 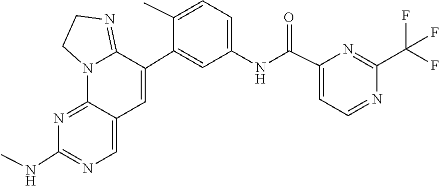 [SMILES: CNc1ncc2c(n1)N1CCN=C1C(c1cc(NC(=O)c3ccnc(C(F)(F)F)n3)ccc1C)=C2] | N-(4-methyl-3-(2- (methylamino)-8,9- dihydroimidazo[1′2′:1,6] pyrido[2,3-d]pyrimidin- 6-yl)phenyl)-2- (trifluoromethyl) pyrimidine-4- carboxamide |
| 148 | 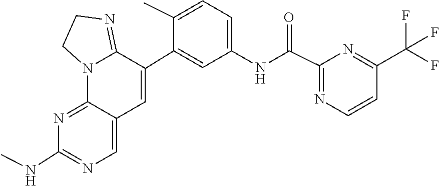 [SMILES: CNc1ncc2c(n1)N1CCN=C1C(c1cc(NC(=O)c3nccc(C(F)(F)F)n3)ccc1C)=C2] | N-(4-methyl-3-(2- (methylamino)-8,9- dihydroimidazo[1′2′:1,6] pyrido[2,3-d]pyrimidin- 6-yl)phenyl)-4- (trifluoromethyl) pyrimidine-2- carboxamide |
| 149 | 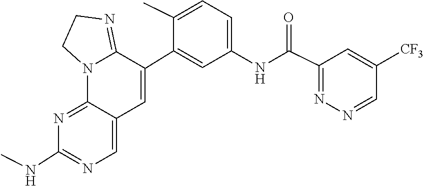 [SMILES: CNc1ncc2c(n1)N1CCN=C1C(c1cc(NC(=O)c3cc(C(F)(F)F)cnn3)ccc1C)=C2] | N-(4-methyl-3-(2- (methylamino)-8,9- dihydroimidazo[1′2′:1,6] pyrido[2,3-d]pyrimidin- 6-yl)phenyl)-5- (trifluoromethyl) pyridazine-3- carboxamide |
| 150 | 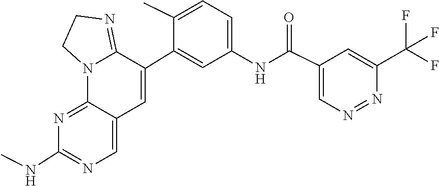 [SMILES: CNc1ncc2c(n1)N1CCN=C1C(c1cc(NC(=O)c3cnnc(C(F)(F)F)c3)ccc1C)=C2] | N-(4-methyl-3-(2- (methylamino)-8,9- dihydroimidazo[1′2′:1,6] pyrido[2,3-d]pyrimidin- 6-yl)phenyl)-6- (trifluoromethyl) pyridazine-4- carboxamide |
| 151 | 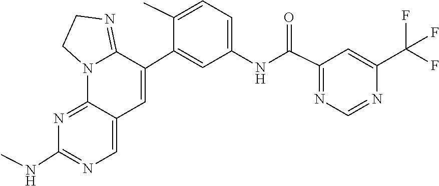 [SMILES: CNc1ncc2c(n1)N1CCN=C1C(c1cc(NC(=O)c3cc(C(F)(F)F)ncn3)ccc1C)=C2] | N-(4-methyl-3-(2- (methylamino)-8,9- dihydroimidazo[1′2′:1,6] pyrido[2,3-d]pyrimidin- 6-yl)phenyl)-6- (trifluoromethyl) pyrimidine-4- carboxamide |
| 152 | 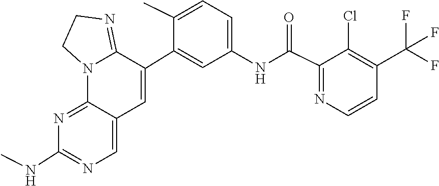 [SMILES: CNc1ncc2c(n1)N1CCN=C1C(c1cc(NC(=O)c3nccc(C(F)(F)F)c3Cl)ccc1C)=C2] | 3-chloro-N-(4-methyl-3- (2-(methylamino)-8,9- dihydroimidazo[1′2′:1,6] pyrido[2,3-d]pyrimidin- 6-yl)phenyl)-4- (trifluoromethyl) picolinamide |
| 153 | 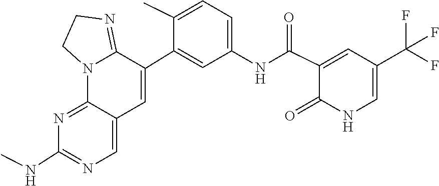 [SMILES: CNc1ncc2c(n1)N1CCN=C1C(c1cc(NC(=O)c3cc(C(F)(F)F)c[nH]c3=O)ccc1C)=C2] | N-(4-methyl-3-(2- (methylamino)-8,9- dihydroimidazo[1′2′:1,6] pyrido[2,3-d]pyrimidin- 6-yl)phenyl)-2-oxo-5- (trifluoromethyl)-1,2- dihydropyridine-3- carboxamide |
| 154 | 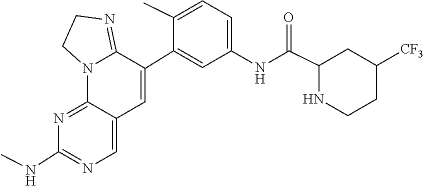 [SMILES: CNc1ncc2c(n1)N1CCN=C1C(c1cc(NC(=O)C3CC(C(F)(F)F)CCN3)ccc1C)=C2] | N-(4-methyl-3-(2- (methylamino)-8,9- dihydroimidazo[1′2′:1,6] pyrido[2,3-d]pyrimidin- 6-yl)phenyl)-4- (trifluoromethyl) piperidine-2- carboxamide |
| 155 | 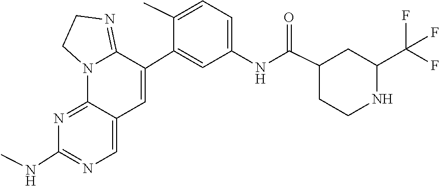 [SMILES: CNc1ncc2c(n1)N1CCN=C1C(c1cc(NC(=O)C3CCNC(C(F)(F)F)C3)ccc1C)=C2] | N-(4-methyl-3-(2- (methylamino)-8,9- dihydroimidazo[1′2′:1,6] pyrido[2,3-d]pyrimidin- 6-yl)phenyl)-2- (trifluoromethyl) piperidine-4- carboxamide |
| 156 | 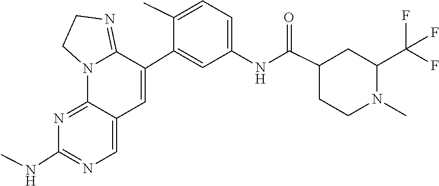 [SMILES: CNc1ncc2c(n1)N1CCN=C1C(c1cc(NC(=O)C3CCN(C)C(C(F)(F)F)C3)ccc1C)=C2] | 1-methyl-N-(4-methyl-3- (2-(methylamino)-8,9- dihydroimidazo[1′2′:1,6] pyrido[2,3-d]pyrimidin- 6-yl)phenyl)-2- (trifluoromethyl) piperidine- 4-carboxamide |
| 157 | 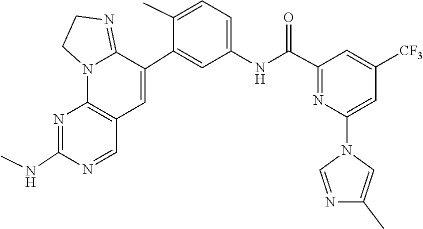 [SMILES: CNc1ncc2c(n1)N1CCN=C1C(c1cc(NC(=O)c3cc(C(F)(F)F)cc(-n4cnc(C)c4)n3)ccc1C)=C2] | 6-(4-methyl-1H- imidazol-1-yl)-N-(4- methyl-3-(2- (methylamino)-8,9- dihydroimidazo[1′2′:1,6] pyrido[2,3-d]pyrimidin- 6-yl)phenyl)-4- (trifluoromethyl) picolinamide |
| 158 | 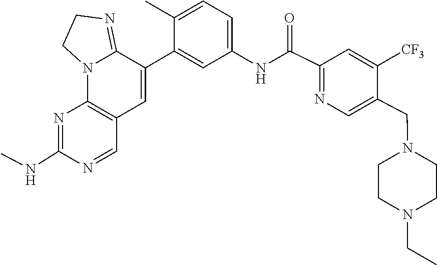 [SMILES: CCN1CCN(Cc2cnc(C(=O)Nc3ccc(C)c(C4=Cc5cnc(NC)nc5N5CCN=C45)c3)cc2C(F)(F)F)CC1] | 5-((4-ethylpiperazin-1- yl)methyl)-N-(4-methyl- 3-(2-(methylamino)-8,9- dihydroimidazo[1′2′:1,6] pyrido[2,3-d]pyrimidin- 6-yl)phenyl)-4- (trifluoromethyl) picolinamide |
| 159 | 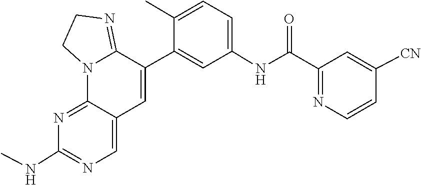 [SMILES: CNc1ncc2c(n1)N1CCN=C1C(c1cc(NC(=O)c3cc(C#N)ccn3)ccc1C)=C2] | 4-cyano-N-(4-methyl-3- (2-(methylamino)-8,9- dihydroimidazo[1′2′:1,6] pyrido[2,3-d]pyrimidin- 6-yl)phenyl)picolinamide |
| 160 | 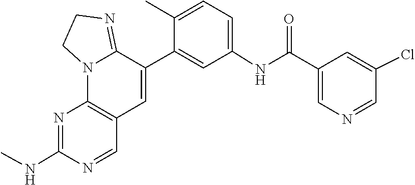 [SMILES: CNc1ncc2c(n1)N1CCN=C1C(c1cc(NC(=O)c3cncc(Cl)c3)ccc1C)=C2] | 5-chloro-N-(4-methyl-3- (2-(methylamino)-8,9- dihydroimidazo[1′2′:1,6] pyrido[2,3-d]pyrimidin- 6-yl)phenyl)nicotinamide |
| 161 | 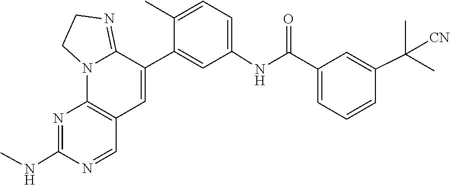 [SMILES: CNc1ncc2c(n1)N1CCN=C1C(c1cc(NC(=O)c3cccc(C(C)(C)C#N)c3)ccc1C)=C2] | 3-(2-cyanopropan-2-yl)- N-(4-methyl-3-(2- (methylamino)-8,9- dihydroimidazo[1′2′:1,6] pyrido[2,3-d]pyrimidin- 6-yl)phenyl)benzamide |
| 162 | 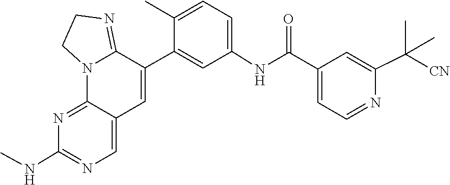 [SMILES: CNc1ncc2c(n1)N1CCN=C1C(c1cc(NC(=O)c3ccnc(C(C)(C)C#N)c3)ccc1C)=C2] | 2-(2-cyanopropan-2-yl)- N-(4-methyl-3-(2- (methylamino)-8,9- dihydroimidazo[1′2′:1,6] pyrido[2,3-d]pyrimidin- 6- yl)phenyl) isonicotinamide |
| 163 | 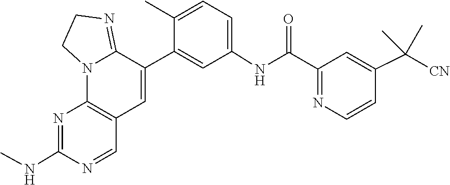 [SMILES: CNc1ncc2c(n1)N1CCN=C1C(c1cc(NC(=O)c3cc(C(C)(C)C#N)ccn3)ccc1C)=C2] | 4-(2-cyanopropan-2-yl)- N-(4-methyl-3-(2- (methylamino)-8,9- dihydroimidazo[1′2′:1,6] pyrido[2,3-d]pyrimidin- 6-yl)phenyl)picolinamide |
| 164 | 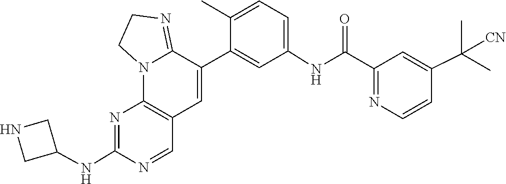 [SMILES: Cc1ccc(NC(=O)c2cc(C(C)(C)C#N)ccn2)cc1C1=Cc2cnc(NC3CNC3)nc2N2CCN=C12] | N-(3-(2-(azetidin-3- ylamino)-8,9- dihydroimidazo[1′2′:1,6] pyrido[2,3-d]pyrimidin- 6-yl)-4-methylpheny1)-4- (2-cyanopropan-2- yl)picolinamide |
| 165 | 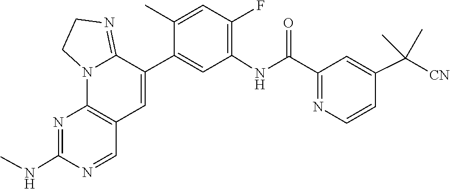 [SMILES: CNc1ncc2c(n1)N1CCN=C1C(c1cc(NC(=O)c3cc(C(C)(C)C#N)ccn3)c(F)cc1C)=C2] | 4-(2-cyanopropan-2-yl)- N-(2-fluoro-4-methyl-5- (2-(methylamino)-8,9- dihydroimidazo[1′2′:1,6] pyrido[2,3-d]pyrimidin- 6-yl)phenyl)picolinamide |
| 166 | 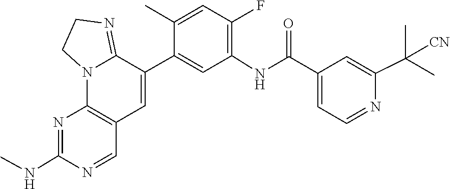 [SMILES: CNc1ncc2c(n1)N1CCN=C1C(c1cc(NC(=O)c3ccnc(C(C)(C)C#N)c3)c(F)cc1C)=C2] | 2-(2-cyanopropan-2-yl)- N-(2-fluoro-4-methyl-5- (2-(methylamino)-8,9- dihydroimidazo[1′2′:1,6] pyrido[2,3-d]pyrimidin- 6- yl)phenyl) isonicotinamide |
| 167 | 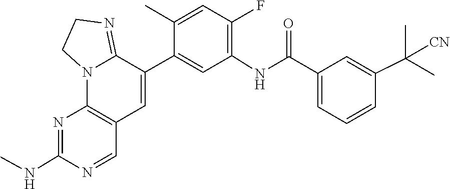 [SMILES: CNc1ncc2c(n1)N1CCN=C1C(c1cc(NC(=O)c3cccc(C(C)(C)C#N)c3)c(F)cc1C)=C2] | 3-(2-cyanopropan-2-yl)- N-(2-fluoro-4-methyl-5- (2-(methylamino)-8,9- dihydroimidazo[1′2′:1,6] pyrido[2,3-d]pyrimidin- 6-yl)phenyl)benzamide |
| 168 | 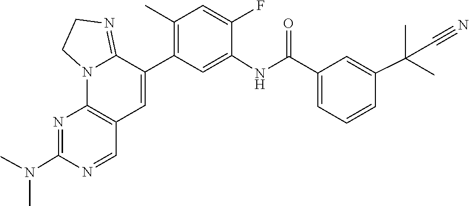 [SMILES: Cc1cc(F)c(NC(=O)c2cccc(C(C)(C)C#N)c2)cc1C1=Cc2cnc(N(C)C)nc2N2CCN=C12] | 3-(2-cyanopropan-2-yl)- N-(5-(2- (dimethylamino)-8,9- dihydroimidazo[1′2′:1,6] pyrido[2,3-d]pyrimidin- 6-yl)-2-fluoro-4- methylphenyl)benzamide |
| 170 | 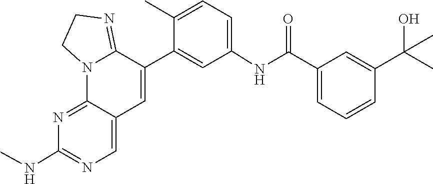 [SMILES: CNc1ncc2c(n1)N1CCN=C1C(c1cc(NC(=O)c3cccc(C(C)(C)O)c3)ccc1C)=C2] | 3-(2-hydroxypropan-2- yl)-N-(4-methyl-3-(2- (methylamino)-8,9- dihydroimidazo[1′2′:1,6] pyrido[2,3-d]pyrimidin- 6-yl)phenyl)benzamide |
| 171 | 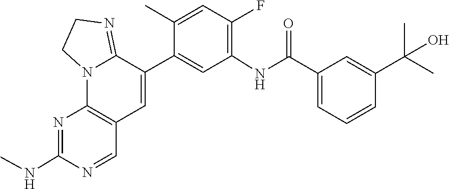 [SMILES: CNc1ncc2c(n1)N1CCN=C1C(c1cc(NC(=O)c3cccc(C(C)(C)O)c3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-3-(2- hydroxypropan-2- yl)benzamide |
| 172 | 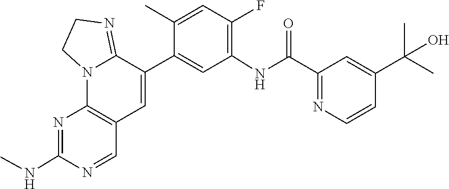 [SMILES: CNc1ncc2c(n1)N1CCN=C1C(c1cc(NC(=O)c3cc(C(C)(C)O)ccn3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-4-(2- hydroxypropan-2- yl)picolinamide |
| 173 | 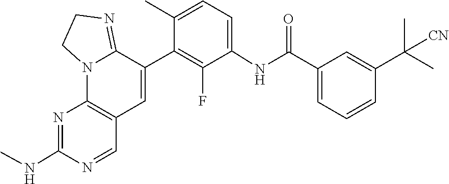 [SMILES: CNc1ncc2c(n1)N1CCN=C1C(c1c(C)ccc(NC(=O)c3cccc(C(C)(C)C#N)c3)c1F)=C2] | 3-(2-cyanopropan-2-yl)- N-(2-fluoro-4-methyl-3- (2-(methylamino)-8,9- dihydroimidazo[1′2′:1,6] pyrido[2,3-d]pyrimidin- 6-yl)phenyl)benzamide |
| 174 | 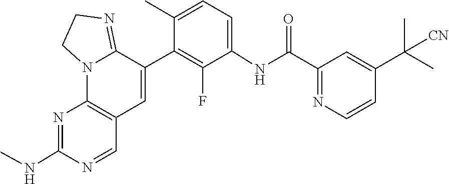 [SMILES: CNc1ncc2c(n1)N1CCN=C1C(c1c(C)ccc(NC(=O)c3cc(C(C)(C)C#N)ccn3)c1F)=C2] | 4-(2-cyanopropan-2-yl)- N-(2-fluoro-4-methyl-3- (2-(methylamino)-8,9- dihydroimidazo[1′2′:1,6] pyrido[2,3-d]pyrimidin- 6-yl)phenyl)picolinamide |
| 175 | 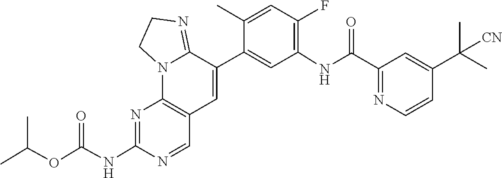 [SMILES: Cc1cc(F)c(NC(=O)c2cc(C(C)(C)C#N)ccn2)cc1C1=Cc2cnc(NC(=O)OC(C)C)nc2N2CCN=C12] | isopropyl (6-(5-(4-(2- cyanopropan-2- yl)picolinamido)-4- fluoro-2-methylphenyl)- 8,9- dihydroimidazo[1′2′:1,6] pyrido[2,3-d]pyrimidin- 2-yl)carbamate |
| 176 | 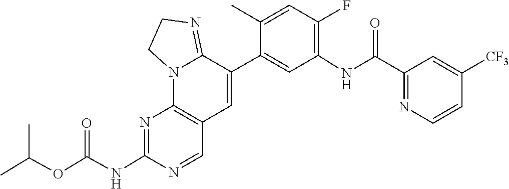 [SMILES: Cc1cc(F)c(NC(=O)c2cc(C(F)(F)F)ccn2)cc1C1=Cc2cnc(NC(=O)OC(C)C)nc2N2CCN=C12] | isopropyl (6-(4-fluoro-2- methyl-5-(4- (trifluoromethyl)pico- linamido)phenyl)-8,9- dihydroimidazo[1′2′:1,6] pyrido[2,3-d]pyrimidin- 2-yl)carbamate |
| 177 | 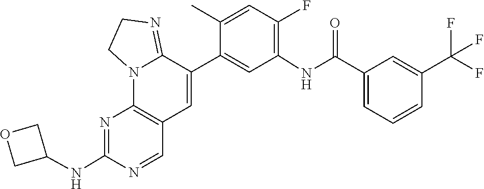 [SMILES: Cc1cc(F)c(NC(=O)c2cccc(C(F)(F)F)c2)cc1C1=Cc2cnc(NC3COC3)nc2N2CCN=C12] | N-(2-fluoro-4-methyl-5- (2-(oxetan-3-ylamino)- 8,9- dihydroimidazo[1′2′:1,6] pyrido[2,3-d]pyrimidin- 6-yl)phenyl)-3- (trifluoromethyl) benzamide |
| 178 | 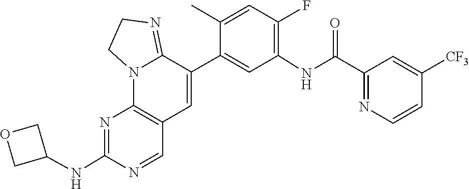 [SMILES: Cc1cc(F)c(NC(=O)c2cc(C(F)(F)F)ccn2)cc1C1=Cc2cnc(NC3COC3)nc2N2CCN=C12] | N-(2-fluoro-4-methyl-5- (2-(oxetan-3-ylamino)- 8,9- dihydroimidazo[1′2′:1,6] pyrido[2,3-d]pyrimidin- 6-yl)phenyl)-4- (trifluoromethyl) picolinamide |
| 179 | 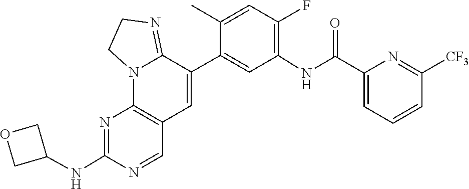 [SMILES: Cc1cc(F)c(NC(=O)c2cccc(C(F)(F)F)n2)cc1C1=Cc2cnc(NC3COC3)nc2N2CCN=C12] | N-(2-fluoro-4-methyl-5- (2-(oxetan-3-ylamino)- 8,9- dihydroimidazo[1′2′:1,6] pyrido[2,3-d]pyrimidin- 6-yl)phenyl)-6- (trifluoromethyl) picolinamide |
| 180 | 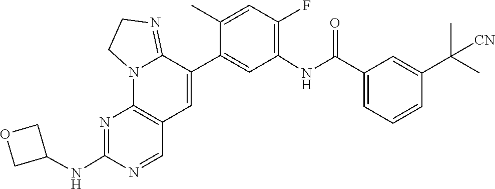 [SMILES: Cc1cc(F)c(NC(=O)c2cccc(C(C)(C)C#N)c2)cc1C1=Cc2cnc(NC3COC3)nc2N2CCN=C12] | 3-(2-cyanopropan-2-yl)- N-(2-fluoro-4-methyl-5- (2-(oxetan-3-ylamino)- 8,9- dihydroimidazo[1′2′:1,6] pyrido[2,3-d]pyrimidin- 6-yl)phenyl)benzamide |
| 181 | 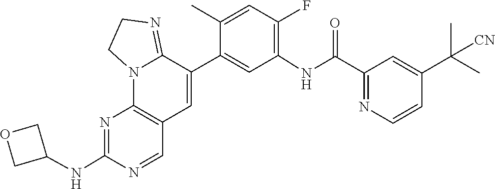 [SMILES: Cc1cc(F)c(NC(=O)c2cc(C(C)(C)C#N)ccn2)cc1C1=Cc2cnc(NC3COC3)nc2N2CCN=C12] | 4-(2-cyanopropan-2-yl)- N-(2-fluoro-4-methyl-5- (2-(oxetan-3-ylamino)- 8,9- dihydroimidazo[1′2′:1,6] pyrido[2,3-d]pyrimidin- 6-yl)phenyl)picolinamide |
| 182 | 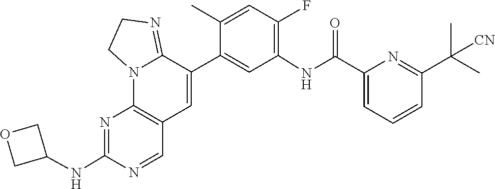 [SMILES: Cc1cc(F)c(NC(=O)c2cccc(C(C)(C)C#N)n2)cc1C1=Cc2cnc(NC3COC3)nc2N2CCN=C12] | 6-(2-cyanopropan-2-yl)- N-(2-fluoro-4-methyl-5- (2-(oxetan-3-ylamino)- 8,9- dihydroimidazo[1′2′:1,6] pyrido[2,3-d]pyrimidin- 6-yl)phenyl)picolinamide |
| 183 | 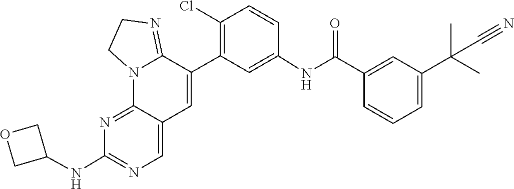 [SMILES: CC(C)(C#N)c1cccc(C(=O)Nc2ccc(Cl)c(C3=Cc4cnc(NC5COC5)nc4N4CCN=C34)c2)c1] | N-(4-chloro-3-(2- (oxetan-3-ylamino)-8,9- dihydroimidazo[1′2′:1,6] pyrido[2,3-d]pyrimidin- 6-yl)phenyl)-3-(2- cyanopropan-2- yl)benzamide |
| 184 | 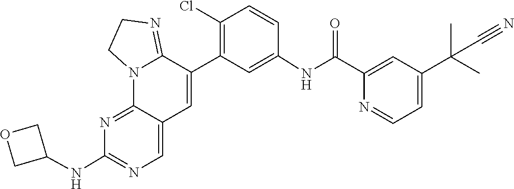 [SMILES: CC(C)(C#N)c1ccnc(C(=O)Nc2ccc(Cl)c(C3=Cc4cnc(NC5COC5)nc4N4CCN=C34)c2)c1] | N-(4-chloro-3-(2- (oxetan-3-ylamino)-8,9- dihydroimidazo[1′2′:1,6] pyrido[2,3-d]pyrimidin- 6-yl)phenyl)-4-(2- cyanopropan-2- yl)picolinamide |
| 185 | 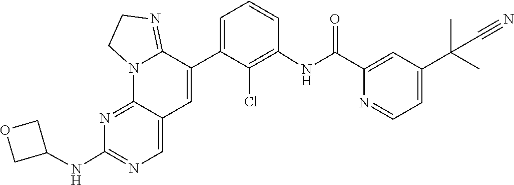 [SMILES: CC(C)(C#N)c1ccnc(C(=O)Nc2cccc(C3=Cc4cnc(NC5COC5)nc4N4CCN=C34)c2Cl)c1] | N-(2-chloro-3-(2- (oxetan-3-ylamino)-8,9- dihydroimidazo[1′2′:1,6] pyrido[2,3-d]pyrimidin- 6-yl)phenyl)-4-(2- cyanopropan-2- yl)picolinamide |
| 186 | 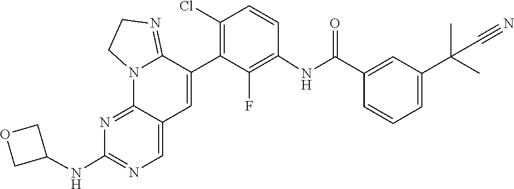 [SMILES: CC(C)(C#N)c1cccc(C(=O)Nc2ccc(Cl)c(C3=Cc4cnc(NC5COC5)nc4N4CCN=C34)c2F)c1] | N-(4-chloro-2-fluoro-3- (2-(oxetan-3-ylamino)- 8,9- dihydroimidazo[1′2′:1,6] pyrido[2,3-d]pyrimidin- 6-yl)phenyl)-3-(2- cyanopropan-2- yl)benzamide |
| 187 | 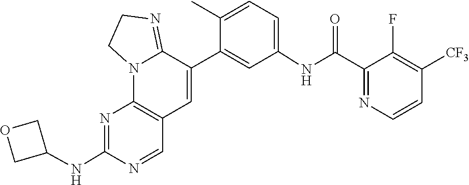 [SMILES: Cc1ccc(NC(=O)c2nccc(C(F)(F)F)c2F)cc1C1=Cc2cnc(NC3COC3)nc2N2CCN=C12] | 3-fluoro-N-(4-methyl-3- (2-(oxetan-3-ylamino)- 8,9- dihydroimidazo[1′2′:1,6] pyrido[2,3-d]pyrimidin- 6-yl)phenyl)-4- (trifluoromethyl) picolinamide |
| 188 | 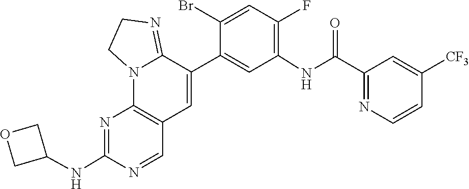 [SMILES: O=C(Nc1cc(C2=Cc3cnc(NC4COC4)nc3N3CCN=C23)c(Br)cc1F)c1cc(C(F)(F)F)ccn1] | N-(4-bromo-2-fluoro-5- (2-(oxetan-3-ylamino)- 8,9- dihydroimidazo[1′2′:1,6] pyrido[2,3-d]pyrimidin- 6-yl)phenyl)-4- (trifluoromethyl) picolinamide |
| 189 | 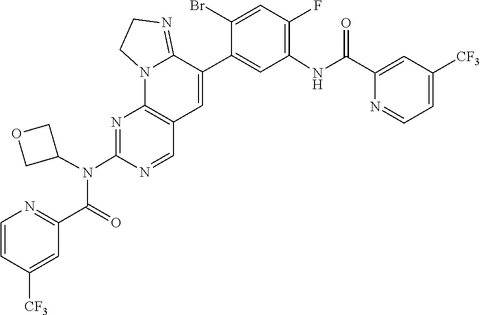 [SMILES: O=C(Nc1cc(C2=Cc3cnc(N(C(=O)c4cc(C(F)(F)F)ccn4)C4COC4)nc3N3CCN=C23)c(Br)cc1F)c1cc(C(F)(F)F)ccn1] | N-(6-(2-bromo-4-fluoro- 5-(4- (trifluoromethyl)pico- linamido)phenyl)-8,9- dihydroimidazo[1′2′:1,6] pyrido[2,3-d]pyrimidin- 2-yl)-N-(oxetan-3-yl)-4- (trifluoromethyl) picolinamide |
| 190 | 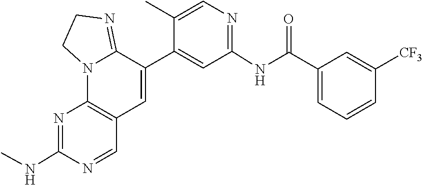 [SMILES: CNc1ncc2c(n1)N1CCN=C1C(c1cc(NC(=O)c3cccc(C(F)(F)F)c3)ncc1C)=C2] | N-(5-methyl-4-(2- (methylamino)-8,9- dihydroimidazo[1′2′:1,6] pyrido[2,3-d]pyrimidin- 6-yl)pyridin-2-yl)-3- (trifluoromethyl) benzamide |
| 191 | 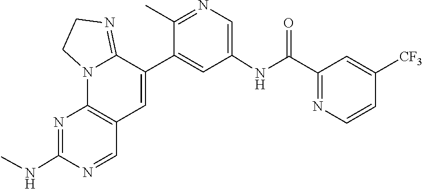 [SMILES: CNc1ncc2c(n1)N1CCN=C1C(c1cc(NC(=O)c3cc(C(F)(F)F)ccn3)cnc1C)=C2] | N-(6-methyl-5-(2- (methylamino)-8,9- dihydroimidazo[1′2′:1,6] pyrido[2,3-d]pyrimidin- 6-yl)pyridin-3-yl)-4- (trifluoromethyl) picolinamide |
| 192 | 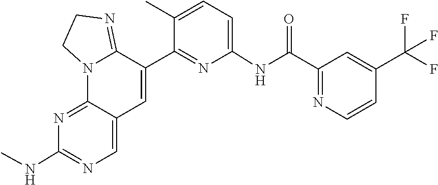 [SMILES: CNc1ncc2c(n1)N1CCN=C1C(c1nc(NC(=O)c3cc(C(F)(F)F)ccn3)ccc1C)=C2] | N-(5-methyl-6-(2- (methylamino)-8,9- dihydroimidazo[1′2′:1,6] pyrido[2,3-d]pyrimidin- 6-yl)pyridin-2-yl)-4- (trifluoromethyl) picolinamide |
| 193 | 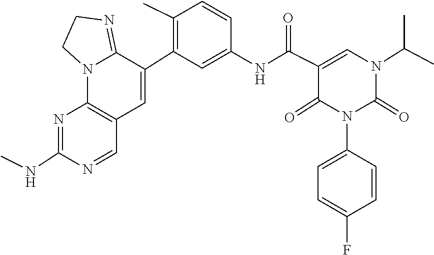 [SMILES: CNc1ncc2c(n1)N1CCN=C1C(c1cc(NC(=O)c3cn(C(C)C)c(=O)n(-c4ccc(F)cc4)c3=O)ccc1C)=C2] | 3-(4-fluorophenyl)-1- isopropyl-N-(4-methyl- 3-(2-(methylamino)-8,9- dihydroimidazo[1′2′:1,6] pyrido[2,3-d]pyrimidin- 6-yl)phenyl)-2,4-dioxo- 1,2,3,4- tetrahydropyrimidine-5- carboxamide |
| 194 | 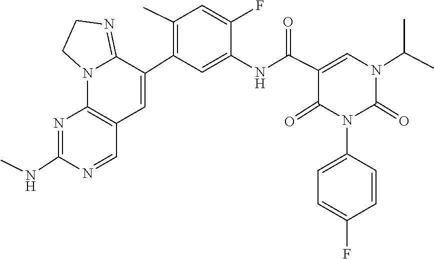 [SMILES: CNc1ncc2c(n1)N1CCN=C1C(c1cc(NC(=O)c3cn(C(C)C)c(=O)n(-c4ccc(F)cc4)c3=O)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-3-(4- fluorophenyl)-1- isopropyl-2,4-dioxo- 1,2,3,4- tetrahydropyrimidine-5- carboxamide |
| 195 | 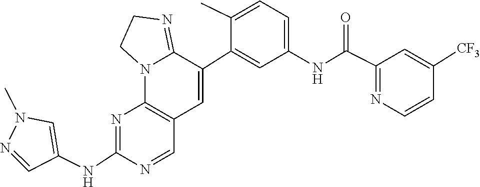 [SMILES: Cc1ccc(NC(=O)c2cc(C(F)(F)F)ccn2)cc1C1=Cc2cnc(Nc3cnn(C)c3)nc2N2CCN=C12] | N-(4-methyl-3-(2-((1- methyl-1H-pyrazol-4- yl)amino)-8,9- dihydroimidazo[1′2′:1,6] pyrido[2,3-d]pyrimidin- 6-yl)phenyl)-4- (trifluoromethyl) picolinamide |
| 196 | 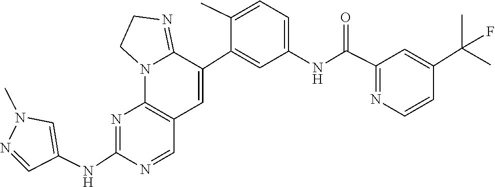 [SMILES: Cc1ccc(NC(=O)c2cc(C(C)(C)F)ccn2)cc1C1=Cc2cnc(Nc3cnn(C)c3)nc2N2CCN=C12] | 4-(2-fluoropropan-2-yl)- N-(4-methyl-3-(2-((1- methyl-1H-pyrazol-4- yl)amino)-8,9- dihydroimidazo[1′2′:1,6] pyrido[2,3-d]pyrimidin- 6-yl)phenyl)picolinamide |
| 197 | 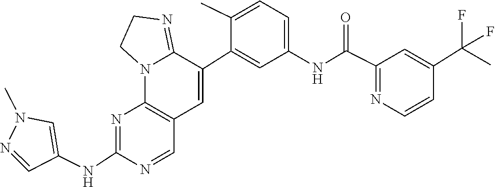 [SMILES: Cc1ccc(NC(=O)c2cc(C(C)(F)F)ccn2)cc1C1=Cc2cnc(Nc3cnn(C)c3)nc2N2CCN=C12] | 4-(1,1-difluoroethyl)-N- (4-methyl-3-(2-((1- methyl-1H-pyrazol-4- yl)amino)-8,9- dihydroimidazo[1′2′:1,6] pyrido[2,3-d]pyrimidin- 6-yl)phenyl)picolinamide |
| 198 | 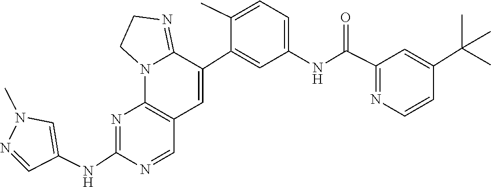 [SMILES: Cc1ccc(NC(=O)c2cc(C(C)(C)C)ccn2)cc1C1=Cc2cnc(Nc3cnn(C)c3)nc2N2CCN=C12] | 4-(tert-butyl)-N-(4- methyl-3-(2-((1-methyl- 1H-pyrazol-4-yl)amino)- 8,9- dihydroimidazo[1′2′:1,6] pyrido[2,3-d]pyrimidin- 6-yl)phenyl)picolinamide |
| 199 | 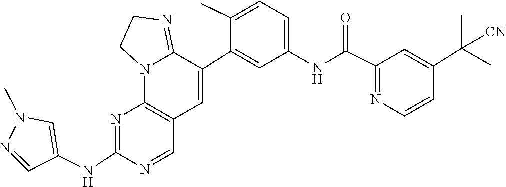 [SMILES: Cc1ccc(NC(=O)c2cc(C(C)(C)C#N)ccn2)cc1C1=Cc2cnc(Nc3cnn(C)c3)nc2N2CCN=C12] | 4-(2-cyanopropan-2-yl)- N-(4-methyl-3-(2-((1- methyl-1H-pyrazol-4- yl)amino)-8,9- dihydroimidazo[1′2′:1,6] pyrido[2,3-d]pyrimidin- 6-yl)phenyl)picolinamide |
| 200 | 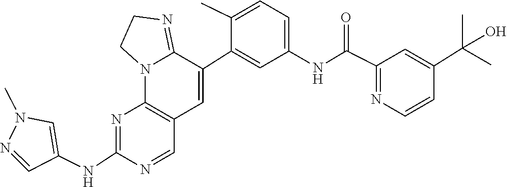 [SMILES: Cc1ccc(NC(=O)c2cc(C(C)(C)O)ccn2)cc1C1=Cc2cnc(Nc3cnn(C)c3)nc2N2CCN=C12] | 4-(2-hydroxypropan-2- yl)-N-(4-methyl-3-(2- ((1-methyl-1H-pyrazol- 4-yl)amino)-8,9- dihydroimidazo[1′2′:1,6] pyrido[2,3-d]pyrimidin- 6-yl)phenyl)picolinamide |
| 201 | 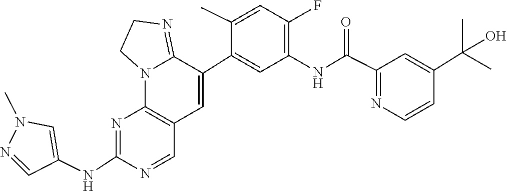 [SMILES: Cc1cc(F)c(NC(=O)c2cc(C(C)(C)O)ccn2)cc1C1=Cc2cnc(Nc3cnn(C)c3)nc2N2CCN=C12] | N-(2-fluoro-4-methyl-5- (2-((1-methyl-1H- pyrazol-4-yl)amino)-8,9- dihydroimidazo[1′2′:1,6] pyrido[2,3-d]pyrimidin- 6-yl)phenyl)-4-(2- hydroxypropan-2- yl)picolinamide |
| 202 | 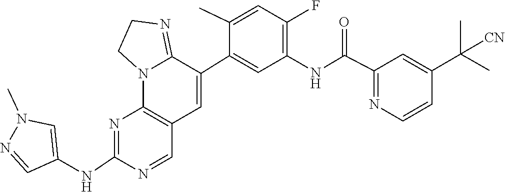 [SMILES: Cc1cc(F)c(NC(=O)c2cc(C(C)(C)C#N)ccn2)cc1C1=Cc2cnc(Nc3cnn(C)c3)nc2N2CCN=C12] | 4-(2-cyanopropan-2-yl)- N-(2-fluoro-4-methyl-5- (2-((1-methyl-1H- pyrazol-4-yl)amino)-8,9- dihydroimidazo[1′2′:1,6] pyrido[2,3-d]pyrimidin- 6-yl)phenyl)picolinamide |
| 203 | 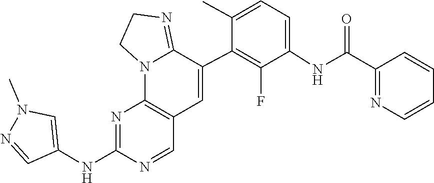 [SMILES: Cc1ccc(NC(=O)c2ccccn2)c(F)c1C1=Cc2cnc(Nc3cnn(C)c3)nc2N2CCN=C12] | N-(2-fluoro-4-methyl-3- (2-((1-methyl-1H- pyrazol-4-yl)amino)-8,9- dihydroimidazo[1′2′:1,6] pyrido[2,3-d]pyrimidin- 6-yl)phenyl)picolinamide |
| 204 | 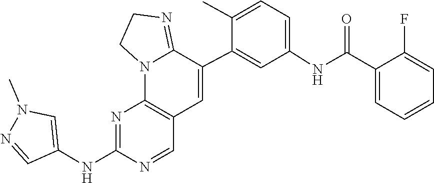 [SMILES: Cc1ccc(NC(=O)c2ccccc2F)cc1C1=Cc2cnc(Nc3cnn(C)c3)nc2N2CCN=C12] | 2-fluoro-N-(4-methyl-3- (2-((1-methyl-1H- pyrazol-4-yl)amino)-8,9- dihydroimidazo[1′2′:1,6] pyrido[2,3-d]pyrimidin- 6-yl)phenyl)benzamide |
| 205 | 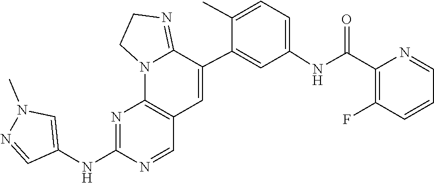 [SMILES: Cc1ccc(NC(=O)c2ncccc2F)cc1C1=Cc2cnc(Nc3cnn(C)c3)nc2N2CCN=C12] | 3-fluoro-N-(4-methyl-3- (2-((1-methyl-1H- pyrazol-4-yl)amino)-8,9- dihydroimidazo[1′2′:1,6] pyrido[2,3-d]pyrimidin- 6-yl)phenyl)picolinamide |
| 206 | 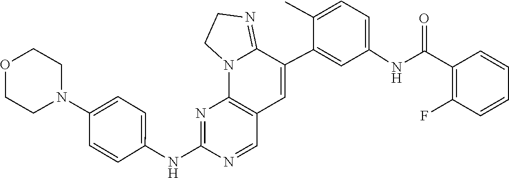 [SMILES: Cc1ccc(NC(=O)c2ccccc2F)cc1C1=Cc2cnc(Nc3ccc(N4CCOCC4)cc3)nc2N2CCN=C12] | 2-fluoro-N-(4-methyl-3- (2-((4- morpholinophenyl) amino)-8,9- dihydroimidazo[1′2′:1,6] pyrido[2,3-d]pyrimidin- 6-yl)phenyl)benzamide |
| 207 | 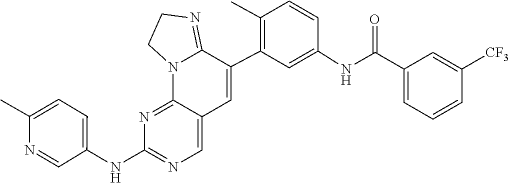 [SMILES: Cc1ccc(Nc2ncc3c(n2)N2CCN=C2C(c2cc(NC(=O)c4cccc(C(F)(F)F)c4)ccc2C)=C3)cn1] | N-(4-methyl-3-(2-((6- methylpyridin-3- yl)amino)-8,9- dihydroimidazo[1′2′:1,6] pyrido[2,3-d]pyrimidin- 6-yl)phenyl)-3- (trifluoromethyl) benzamide |
| 208 | 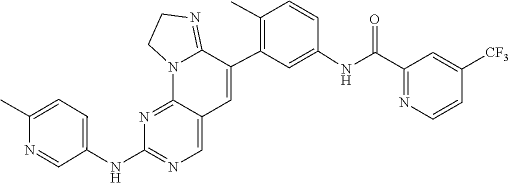 [SMILES: Cc1ccc(Nc2ncc3c(n2)N2CCN=C2C(c2cc(NC(=O)c4cc(C(F)(F)F)ccn4)ccc2C)=C3)cn1] | N-(4-methyl-3-(2-((6- methylpyridin-3- yl)amino)-8,9- dihydroimidazo[1′2′:1,6] pyrido[2,3-d]pyrimidin- 6-yl)phenyl)-4- (trifluoromethyl) picolinamide |
| 209 | 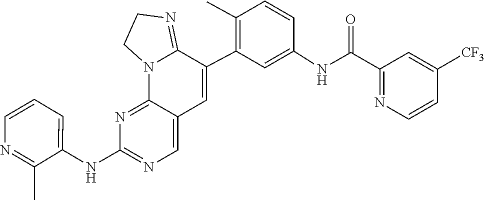 [SMILES: Cc1ccc(NC(=O)c2cc(C(F)(F)F)ccn2)cc1C1=Cc2cnc(Nc3cccnc3C)nc2N2CCN=C12] | N-(4-methyl-3-(2-((2- methylpyridin-3- yl)amino)-8,9- dihydroimidazo[1′2′:1,6] pyrido[2,3-d]pyrimidin- 6-yl)phenyl)-4- (trifluoromethyl) picolinamide |
| 210 | 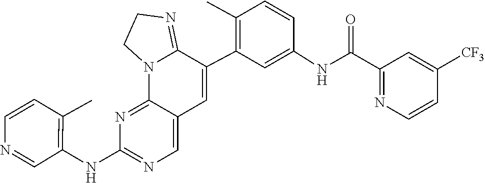 [SMILES: Cc1ccncc1Nc1ncc2c(n1)N1CCN=C1C(c1cc(NC(=O)c3cc(C(F)(F)F)ccn3)ccc1C)=C2] | N-(4-methyl-3-(2-((4- methylpyridin-3- yl)amino)-8,9- dihydroimidazo[1′2′:1,6] pyrido[2,3-d]pyrimidin- 6-yl)phenyl)-4- (trifluoromethyl) picolinamide |
| 211 | 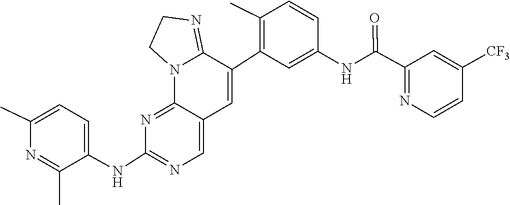 [SMILES: Cc1ccc(Nc2ncc3c(n2)N2CCN=C2C(c2cc(NC(=O)c4cc(C(F)(F)F)ccn4)ccc2C)=C3)c(C)n1] | N-(3-(2-((2,6- dimethylpyridin-3- yl)amino)-8,9- dihydroimidazo[1′2′:1,6] pyrido[2,3-d]pyrimidin- 6-yl)-4-methylphenyl)-4- (trifluoromethyl) picolinamide |
| 212 | 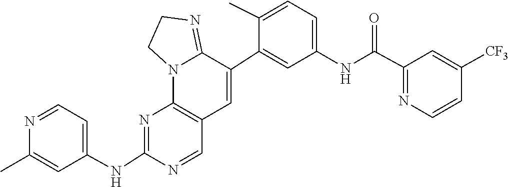 [SMILES: Cc1cc(Nc2ncc3c(n2)N2CCN=C2C(c2cc(NC(=O)c4cc(C(F)(F)F)ccn4)ccc2C)=C3)ccn1] | N-(4-methyl-3-(2-((2- methylpyridin-4- yl)amino)-8,9- dihydroimidazo[1′2′:1,6] pyrido[2,3-d]pyrimidin- 6-yl)phenyl)-4- (trifluoromethyl) picolinamide |
| 213 | 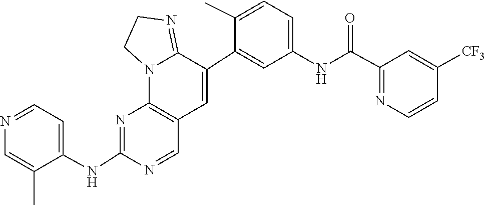 [SMILES: Cc1cnccc1Nc1ncc2c(n1)N1CCN=C1C(c1cc(NC(=O)c3cc(C(F)(F)F)ccn3)ccc1C)=C2] | N-(4-methyl-3-(2-((3- methylpyridin-4- yl)amino)-8,9- dihydroimidazo[1′2′:1,6] pyrido[2,3-d]pyrimidin- 6-yl)phenyl)-4- (trifluoromethyl) picolinamide |
| 214 | 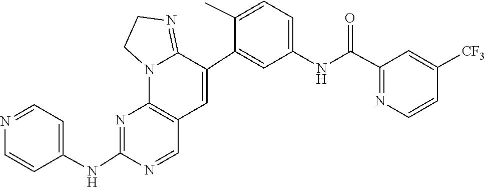 [SMILES: Cc1ccc(NC(=O)c2cc(C(F)(F)F)ccn2)cc1C1=Cc2cnc(Nc3ccncc3)nc2N2CCN=C12] | N-(4-methyl-3-(2- (pyridin-4-ylamino)-8,9- dihydroimidazo[1′2′:1,6] pyrido[2,3-d]pyrimidin- 6-yl)phenyl)-4- (trifluoromethyl) picolinamide |
| 215 | 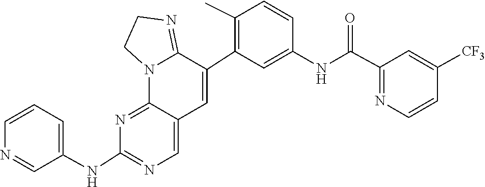 [SMILES: Cc1ccc(NC(=O)c2cc(C(F)(F)F)ccn2)cc1C1=Cc2cnc(Nc3cccnc3)nc2N2CCN=C12] | N-(4-methyl-3-(2- (pyridin-3-ylamino)-8,9- dihydroimidazo[1′2′:1,6] pyrido[2,3-d]pyrimidin- 6-yl)phenyl)-4- (trifluoromethyl) picolinamide |
| 216 | 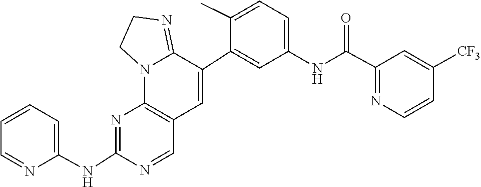 [SMILES: Cc1ccc(NC(=O)c2cc(C(F)(F)F)ccn2)cc1C1=Cc2cnc(Nc3ccccn3)nc2N2CCN=C12] | N-(4-methyl-3-(2- (pyridin-2-ylamino)-8,9- dihydroimidazo[1′2′:1,6] pyrido[2,3-d]pyrimidin- 6-yl)phenyl)-4- (trifluoromethyl) picolinamide |
| 217 | 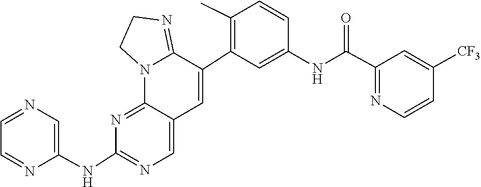 [SMILES: Cc1ccc(NC(=O)c2cc(C(F)(F)F)ccn2)cc1C1=Cc2cnc(Nc3cnccn3)nc2N2CCN=C12] | N-(4-methyl-3-(2- (pyrazin-2-ylamino)-8,9- dihydroimidazo[1′2′:1,6] pyrido[2,3-d]pyrimidin- 6-yl)phenyl)-4- (trifluoromethyl) picolinamide |
| 218 | 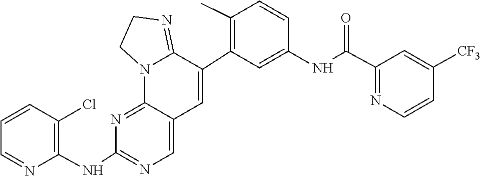 [SMILES: Cc1ccc(NC(=O)c2cc(C(F)(F)F)ccn2)cc1C1=Cc2cnc(Nc3ncccc3Cl)nc2N2CCN=C12] | N-(3-(2-((3- chloropyridin-2- yl)amino)-8,9- dihydroimidazo[1′2′:1,6] pyrido[2,3-d]pyrimidin- 6-yl)-4-methylphenyl)-4- (trifluoromethyl) picolinamide |
| 219 | 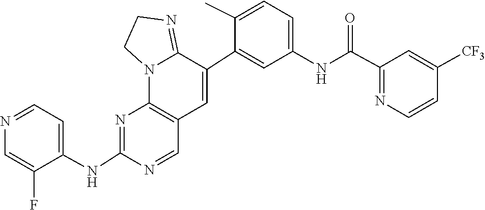 [SMILES: Cc1ccc(NC(=O)c2cc(C(F)(F)F)ccn2)cc1C1=Cc2cnc(Nc3ccncc3F)nc2N2CCN=C12] | N-(3-(2-((3- fluoropyridin-4- yl)amino)-8,9- dihydroimidazo[1′2′:1,6] pyrido[2,3-d]pyrimidin- 6-yl)-4-methylphenyl)-4- (trifluoromethyl) picolinamide |
| 220 | 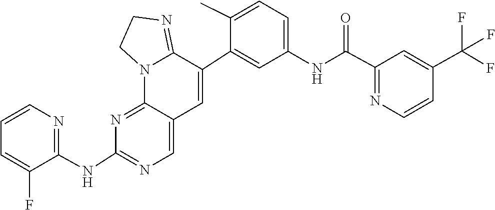 [SMILES: Cc1ccc(NC(=O)c2cc(C(F)(F)F)ccn2)cc1C1=Cc2cnc(Nc3ncccc3F)nc2N2CCN=C12] | N-(3-(2-((3- fluoropyridin-2- yl)amino)-8,9- dihydroimidazo[1′2′:1,6] pyrido[2,3-d]pyrimidin- 6-yl)-4-methylphenyl)-4- (trifluoromethyl) picolinamide |
| 221 | 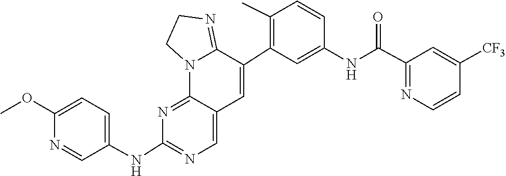 [SMILES: COc1ccc(Nc2ncc3c(n2)N2CCN=C2C(c2cc(NC(=O)c4cc(C(F)(F)F)ccn4)ccc2C)=C3)cn1] | N-(3-(2-((6- methoxypyridin-3- yl)amino)-8,9- dihydroimidazo[1′2′:1,6] pyrido[2,3-d]pyrimidin- 6-yl)-4-methylphenyl)-4- (trifluoromethyl) picolinamide |
| 222 | 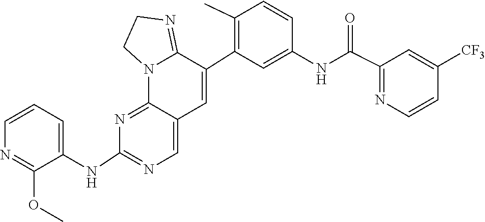 [SMILES: COc1ncccc1Nc1ncc2c(n1)N1CCN=C1C(c1cc(NC(=O)c3cc(C(F)(F)F)ccn3)ccc1C)=C2] | N-(3-(2-((2- methoxypyridin-3- yl)amino)-8,9- dihydroimidazo[1′2′:1,6] pyrido[2,3-d]pyrimidin- 6-yl)-4-methylphenyl)-4- (trifluoromethyl) picolinamide |
| 223 | 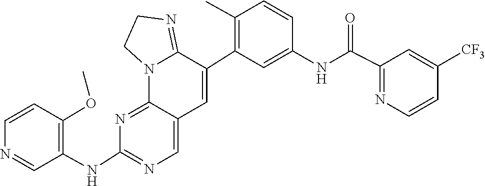 [SMILES: COc1ccncc1Nc1ncc2c(n1)N1CCN=C1C(c1cc(NC(=O)c3cc(C(F)(F)F)ccn3)ccc1C)=C2] | N-(3-(2-((4- methoxypyridin-3- yl)amino)-8,9- dihydroimidazo[1′2′:1,6] pyrido[2,3-d]pyrimidin- 6-yl)-4-methylphenyl)-4- (trifluoromethyl) picolinamide |
| 224 | 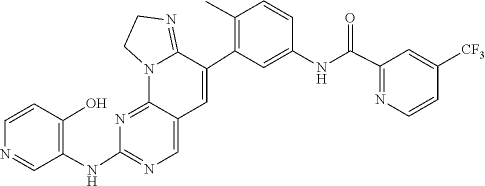 [SMILES: Cc1ccc(NC(=O)c2cc(C(F)(F)F)ccn2)cc1C1=Cc2cnc(Nc3cnccc3O)nc2N2CCN=C12] | N-(3-(2-((4- hydroxypyridin-3- yl)amino)-8,9- dihydroimidazo[1′2′:1,6] pyrido[2,3-d]pyrimidin- 6-yl)-4-methylphenyl)-4- (trifluoromethyl) picolinamide |
| 225 | 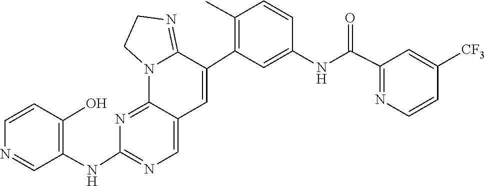 [SMILES: Cc1ccc(NC(=O)c2cc(C(F)(F)F)ccn2)cc1C1=Cc2cnc(Nc3cnccc3O)nc2N2CCN=C12] | N-(3-(2-((2- fluorophenyl)amino)-8,9- dihydroimidazo[1′2′:1,6] pyrido[2,3-d]pyrimidin- 6-yl)-4-methylphenyl)-4- (trifluoromethyl) picolinamide |
| 226 | 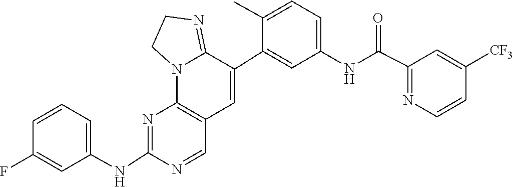 [SMILES: Cc1ccc(NC(=O)c2cc(C(F)(F)F)ccn2)cc1C1=Cc2cnc(Nc3cccc(F)c3)nc2N2CCN=C12] | N-(3-(2-((3- fluorophenyl)amino)-8,9- dihydroimidazo[1′2′:1,6] pyrido[2,3-d]pyrimidin- 6-yl)-4-methylphenyl)-4- (trifluoromethyl) picolinamide |
| 227 | 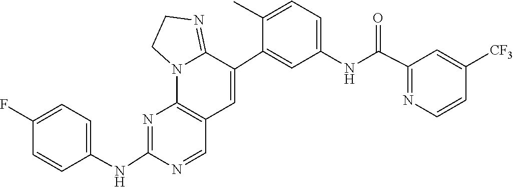 [SMILES: Cc1ccc(NC(=O)c2cc(C(F)(F)F)ccn2)cc1C1=Cc2cnc(Nc3ccc(F)cc3)nc2N2CCN=C12] | N-(3-(2-((4- fluorophenyl)amino)-8,9- dihydroimidazo[1′2′:1,6] pyrido[2,3-d]pyrimidin- 6-yl)-4-methylphenyl)-4- (trifluoromethyl) picolinamide |
| 228 | 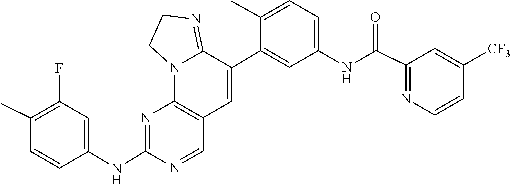 [SMILES: Cc1ccc(Nc2ncc3c(n2)N2CCN=C2C(c2cc(NC(=O)c4cc(C(F)(F)F)ccn4)ccc2C)=C3)cc1F] | N-(3-(2-((3-fluoro-4- methylphenyl)amino)- 8,9- dihydroimidazo[1′2′:1,6] pyrido[2,3-d]pyrimidin- 6-yl)-4-methylphenyl)-4- (trifluoromethyl) picolinamide |
| 229 | 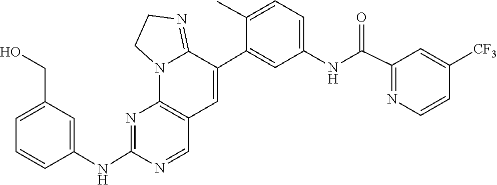 [SMILES: Cc1ccc(NC(=O)c2cc(C(F)(F)F)ccn2)cc1C1=Cc2cnc(Nc3cccc(CO)c3)nc2N2CCN=C12] | N-(3-(2-((3- (hydroxymethyl)phenyl) amino)-8,9- dihydroimidazo[1′2′:1,6] pyrido[2,3-d]pyrimidin- 6-yl)-4-methylphenyl)-4- (trifluoromethyl) picolinamide |
| 230 | 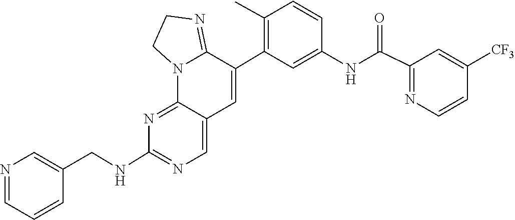 [SMILES: Cc1ccc(NC(=O)c2cc(C(F)(F)F)ccn2)cc1C1=Cc2cnc(NCc3cccnc3)nc2N2CCN=C12] | N-(4-methyl-3-(2- ((pyridin-3- ylmethyl)amino)-8,9- dihydroimidazo[1′2′:1,6] pyrido[2,3-d]pyrimidin- 6-yl)phenyl)-4- (trifluoromethyl) picolinamide |
| 231 | 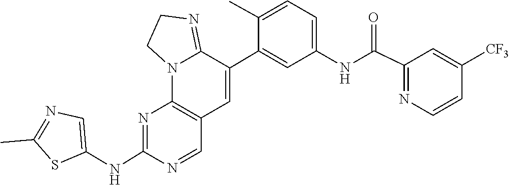 [SMILES: Cc1ncc(Nc2ncc3c(n2)N2CCN=C2C(c2cc(NC(=O)c4cc(C(F)(F)F)ccn4)ccc2C)=C3)s1] | N-(4-methyl-3-(2-((2- methylthiazol-5- yl)amino)-8,9- dihydroimidazo[1′2′:1,6] pyrido[2,3-d]pyrimidin- 6-yl)phenyl)-4- (trifluoromethyl) picolinamide |
| 232 | 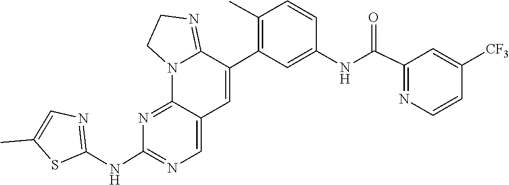 [SMILES: Cc1cnc(Nc2ncc3c(n2)N2CCN=C2C(c2cc(NC(=O)c4cc(C(F)(F)F)ccn4)ccc2C)=C3)s1] | N-(4-methyl-3-(2-((5- methylthiazol-2- yl)amino)-8,9- dihydroimidazo[1′2′:1,6] pyrido[2,3-d]pyrimidin- 6-yl)phenyl)-4- (trifluoromethyl) picolinamide |
| 233 | 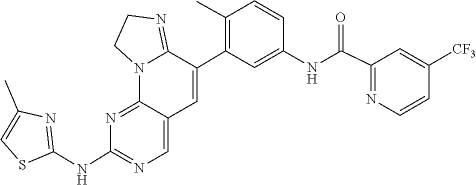 [SMILES: Cc1csc(Nc2ncc3c(n2)N2CCN=C2C(c2cc(NC(=O)c4cc(C(F)(F)F)ccn4)ccc2C)=C3)n1] | N-(4-methyl-3-(2-((4- methylthiazol-2- yl)amino)-8,9- dihydroimidazo[1′2′:1,6] pyrido[2,3-d]pyrimidin- 6-yl)phenyl)-4- (trifluoromethyl) picolinamide |
| 234 | 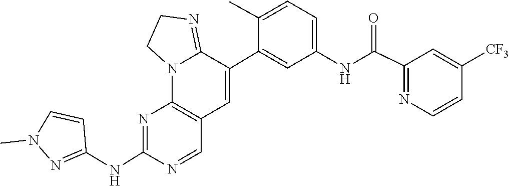 [SMILES: Cc1ccc(NC(=O)c2cc(C(F)(F)F)ccn2)cc1C1=Cc2cnc(Nc3ccn(C)n3)nc2N2CCN=C12] | N-(4-methyl-3-(2-((1- methyl-1H-pyrazol-3- yl)amino)-8,9- dihydroimidazo[1′2′:1,6] pyrido[2,3-d]pyrimidin- 6-yl)phenyl)-4- (trifluoromethyl) picolinamide |
| 235 | 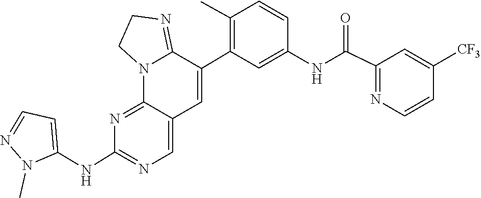 [SMILES: Cc1ccc(NC(=O)c2cc(C(F)(F)F)ccn2)cc1C1=Cc2cnc(Nc3ccnn3C)nc2N2CCN=C12] | N-(4-methyl-3-(2-((1- methyl-1H-pyrazol-5- yl)amino)-8,9- dihydroimidazo[1′2′:1,6] pyrido[2,3-d]pyrimidin- 6-yl)phenyl)-4- (trifluoromethyl) picolinamide |
| 236 | 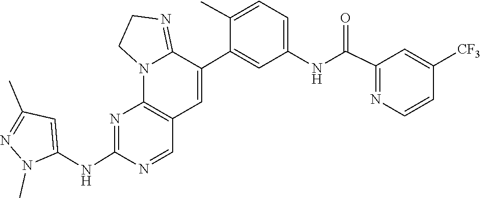 [SMILES: Cc1cc(Nc2ncc3c(n2)N2CCN=C2C(c2cc(NC(=O)c4cc(C(F)(F)F)ccn4)ccc2C)=C3)n(C)n1] | N-(3-(2-((1,3-dimethyl- 1H-pyrazol-5-yl)amino)- 8,9- dihydroimidazo[1′2′:1,6] pyrido[2,3-d]pyrimidin- 6-yl)-4-methylphenyl)-4- (trifluoromethyl) picolinamide |
| 237 | 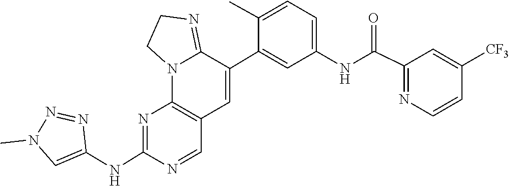 [SMILES: Cc1ccc(NC(=O)c2cc(C(F)(F)F)ccn2)cc1C1=Cc2cnc(Nc3cn(C)nn3)nc2N2CCN=C12] | N-(4-methyl-3-(2-((1- methyl-1H-1,2,3-triazol- 4-yl)amino)-8,9- dihydroimidazo[1′2′:1,6] pyrido[2,3-d]pyrimidin- 6-yl)phenyl)-4- (trifluoromethyl) picolinamide |
| 238 | 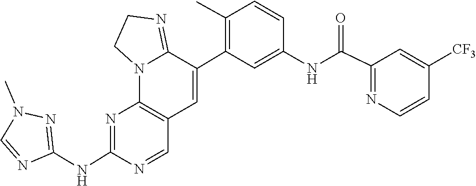 [SMILES: Cc1ccc(NC(=O)c2cc(C(F)(F)F)ccn2)cc1C1=Cc2cnc(Nc3ncn(C)n3)nc2N2CCN=C12] | N-(4-methyl-3-(2-((1- methyl-1H-1,2,4-triazol- 3-yl)amino)-8,9- dihydroimidazo[1′2′:1,6] pyrido[2,3-d]pyrimidin- 6-yl)phenyl)-4- (trifluoromethyl) picolinamide |
| 239 | 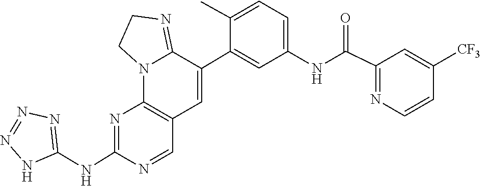 [SMILES: Cc1ccc(NC(=O)c2cc(C(F)(F)F)ccn2)cc1C1=Cc2cnc(Nc3nnn[nH]3)nc2N2CCN=C12] | N-(3-(2-((1H-tetrazol-5- yl)amino)-8,9- dihydroimidazo[1′2′:1,6] pyrido[2,3-d]pyrimidin- 6-yl)-4-methylphenyl)-4- (trifluoromethyl) picolinamide |
| 240 | 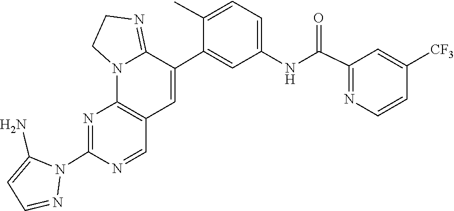 [SMILES: Cc1ccc(NC(=O)c2cc(C(F)(F)F)ccn2)cc1C1=Cc2cnc(-n3nccc3N)nc2N2CCN=C12] | N-(3-(2-((1H-pyrazol-5- yl)amino)-8,9- dihydroimidazo[1′2′:1,6] pyrido[2,3-d]pyrimidin- 6-yl)-4-methylphenyl)-4- (trifluoromethyl) picolinamide |
| 241 | 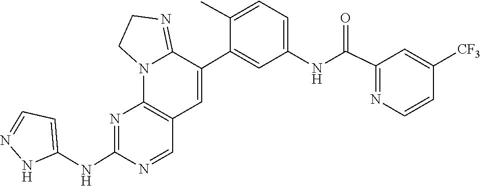 [SMILES: Cc1ccc(NC(=O)c2cc(C(F)(F)F)ccn2)cc1C1=Cc2cnc(Nc3ccn[nH]3)nc2N2CCN=C12] | N-(3-(2-(5-amino-1H- pyrazol-1-yl)-8,9- dihydroimidazo[1′2′:1,6] pyrido[2,3-d]pyrimidin- 6-yl)-4-methylphenyl)-4- (trifluoromethyl) picolinamide |
| 242 | 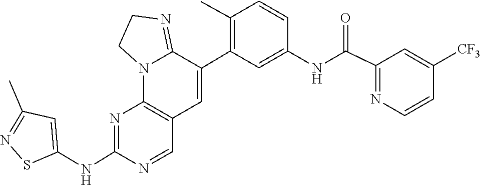 [SMILES: Cc1cc(Nc2ncc3c(n2)N2CCN=C2C(c2cc(NC(=O)c4cc(C(F)(F)F)ccn4)ccc2C)=C3)sn1] | N-(4-methyl-3-(2-((3- methylisothiazol-5- yl)amino)-8,9- dihydroimidazo[1′2′:1,6] pyrido[2,3-d]pyrimidin- 6-yl)phenyl)-4- (trifluoromethyl) picolinamide |
| 243 | 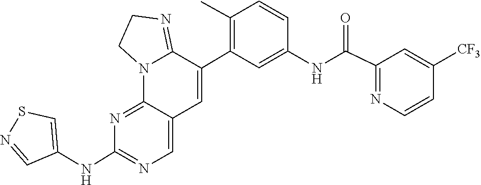 [SMILES: Cc1ccc(NC(=O)c2cc(C(F)(F)F)ccn2)cc1C1=Cc2cnc(Nc3cnsc3)nc2N2CCN=C12] | N-(3-(2-(isothiazol-4- ylamino)-8,9- dihydroimidazo[1′2′:1,6] pyrido[2,3-d]pyrimidin- 6-yl)-4-methylphenyl)-4- (trifluoromethyl) picolinamide |
| 244 | 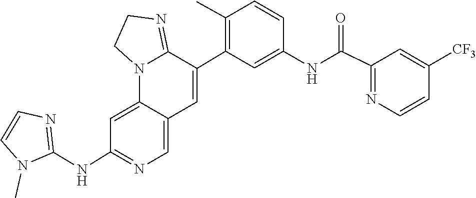 [SMILES: Cc1ccc(NC(=O)c2cc(C(F)(F)F)ccn2)cc1C1=Cc2cnc(Nc3nccn3C)cc2N2CCN=C12] | N-(4-methyl-3-(2-((1- methyl-1H-imidazol-2- yl)amino)-8,9- dihydroimidazo[1′2′:1,6] pyrido[2,3-d]pyrimidin- 6-yl)phenyl)-4- (trifluoromethyl) picolinamide |
| 245 | 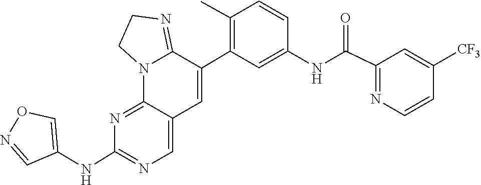 [SMILES: Cc1ccc(NC(=O)c2cc(C(F)(F)F)ccn2)cc1C1=Cc2cnc(Nc3cnoc3)nc2N2CCN=C12] | N-(3-(2-(isoxazol-4- ylamino)-8,9- dihydroimidazo[1′2′:1,6] pyrido[2,3-d]pyrimidin- 6-yl)-4-methylphenyl)-4- (trifluoromethyl) picolinamide |
| 246 | 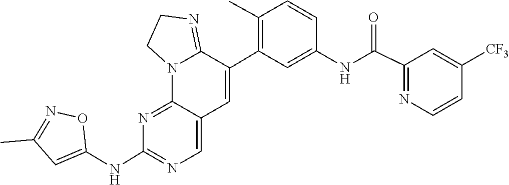 [SMILES: Cc1cc(Nc2ncc3c(n2)N2CCN=C2C(c2cc(NC(=O)c4cc(C(F)(F)F)ccn4)ccc2C)=C3)on1] | N-(4-methyl-3-(2-((3- methylisoxazol-5- yl)amino)-8,9- dihydroimidazo[1′2′:1,6] pyrido[2,3-d]pyrimidin- 6-yl)phenyl)-4- (trifluoromethyl) picolinamide |
| 247 | 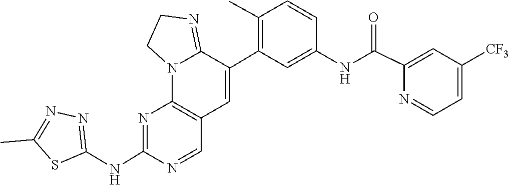 [SMILES: Cc1nnc(Nc2ncc3c(n2)N2CCN=C2C(c2cc(NC(=O)c4cc(C(F)(F)F)ccn4)ccc2C)=C3)s1] | N-(4-methyl-3-(2-((5- methyl-1,3,4-thiadiazol- 2-yl)amino)-8,9- dihydroimidazo[1′2′:1,6] pyrido[2,3-d]pyrimidin- 6-yl)phenyl)-4- (trifluoromethyl) picolinamide |
| 248 | 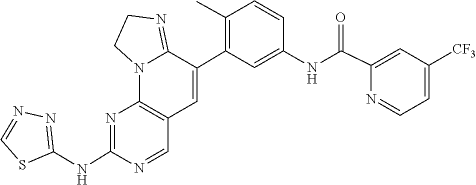 [SMILES: Cc1ccc(NC(=O)c2cc(C(F)(F)F)ccn2)cc1C1=Cc2cnc(Nc3nncs3)nc2N2CCN=C12] | N-(3-(2-((1,3,4- thiadiazol-2-yl)amino)- 8,9- dihydroimidazo[1′2′:1,6] pyrido[2,3-d]pyrimidin- 6-yl)-4-methylphenyl)-4- (trifluoromethyl) picolinamide |
| 249 | 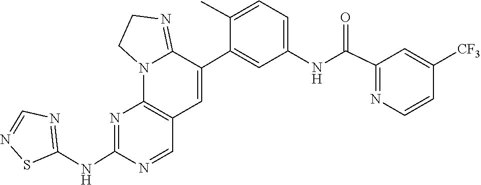 [SMILES: Cc1ccc(NC(=O)c2cc(C(F)(F)F)ccn2)cc1C1=Cc2cnc(Nc3ncns3)nc2N2CCN=C12] | N-(3-(2-((1,2,4- thiadiazol-5-yl)amino)- 8,9- dihydroimidazo[1′2′:1,6] pyrido[2,3-d]pyrimidin- 6-yl)-4-methylpheny1)-4- (trifluoromethyl) picolinamide |
| 250 | 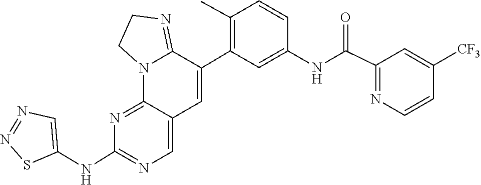 [SMILES: Cc1ccc(NC(=O)c2cc(C(F)(F)F)ccn2)cc1C1=Cc2cnc(Nc3cnns3)nc2N2CCN=C12] | N-(3-(2-((1,2,3- thiadiazol-5-yl)amino)- 8,9- dihydroimidazo[1′2′:1,6] pyrido[2,3-d]pyrimidin- 6-y1)-4-methylphenyl)-4- (trifluoromethyl) picolinamide |
| 251 | 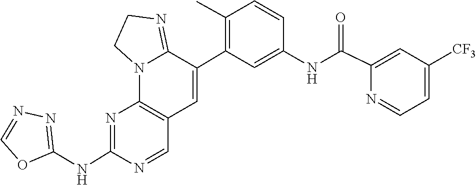 [SMILES: Cc1ccc(NC(=O)c2cc(C(F)(F)F)ccn2)cc1C1=Cc2cnc(Nc3nnco3)nc2N2CCN=C12] | N-(3-(2-((1,3,4- oxadiazol-2-yl)amino)- 8,9- dihydroimidazo[1′2′:1,6] pyrido[2,3-d]pyrimidin- 6-yl)-4-methylphenyl)-4- (trifluoromethyl) picolinamide |
| 252 | 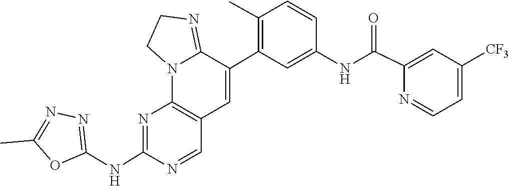 [SMILES: Cc1nnc(Nc2ncc3c(n2)N2CCN=C2C(c2cc(NC(=O)c4cc(C(F)(F)F)ccn4)ccc2C)=C3)o1] | N-(4-methyl-3-(2-((5- methyl-1,3,4-oxadiazol- 2-yl)amino)-8,9- dihydroimidazo[1′2′:1,6] pyrido[2,3-d]pyrimidin- 6-yl)phenyl)-4- (trifluoromethyl) picolinamide |
| 253 | 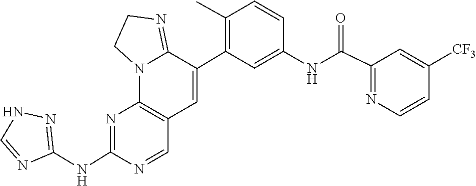 [SMILES: Cc1ccc(NC(=O)c2cc(C(F)(F)F)ccn2)cc1C1=Cc2cnc(Nc3nc[nH]n3)nc2N2CCN=C12] | N-(3-(2-((1H-1,2,4- triazol-3-yl)amino)-8,9- dihydroimidazo[1′2′:1,6] pyrido[2,3-d]pyrimidin- 6-yl)-4-methylphenyl)-4- (trifluoromethyl) picolinamide |
| 254 | 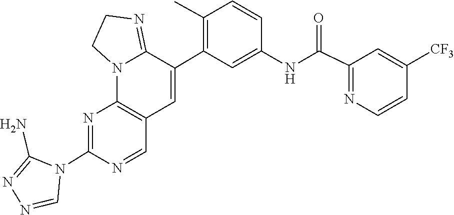 [SMILES: Cc1ccc(NC(=O)c2cc(C(F)(F)F)ccn2)cc1C1=Cc2cnc(-n3cnnc3N)nc2N2CCN=C12] | N-(3-(2-((1H-1,2,4- triazol-3-yl)amino)-8,9- dihydroimidazo[1′2′:1,6] pyrido[2,3-d]pyrimidin- 6-yl)-4-methylphenyl)-4- (trifluoromethyl) picolinamide |
| 255 | 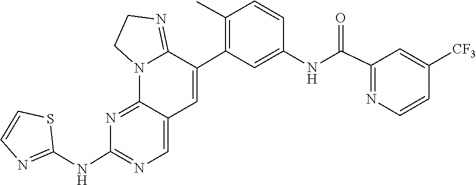 [SMILES: Cc1ccc(NC(=O)c2cc(C(F)(F)F)ccn2)cc1C1=Cc2cnc(Nc3nccs3)nc2N2CCN=C12] | N-(4-methyl-3-(2- (thiazol-2-ylamino)-8,9- dihydroimidazo[1′2′:1,6] pyrido[2,3-d]pyrimidin- 6-yl)phenyl)-4- (trifluoromethyl) picolinamide |
| 256 | 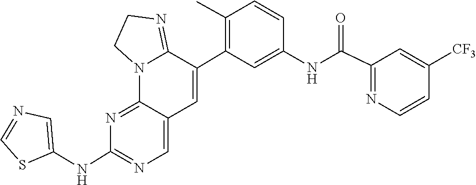 [SMILES: Cc1ccc(NC(=O)c2cc(C(F)(F)F)ccn2)cc1C1=Cc2cnc(Nc3cncs3)nc2N2CCN=C12] | N-(4-methyl-3-(2- (thiazol-5-ylamino)-8,9- dihydroimidazo[1′2′:1,6] pyrido[2,3-d]pyrimidin- 6-yl)phenyl)-4- (trifluoromethyl) picolinamide |
| 257 | 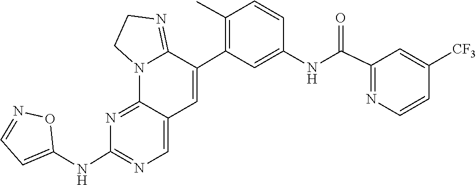 [SMILES: Cc1ccc(NC(=O)c2cc(C(F)(F)F)ccn2)cc1C1=Cc2cnc(Nc3ccno3)nc2N2CCN=C12] | N-(3-(2-(isoxazol-5- ylamino)-8,9- dihydroimidazo[1′2′:1,6] pyrido[2,3-d]pyrimidin- 6-yl)-4-methylphenyl)-4- (trifluoromethyl) picolinamide |
| 258 | 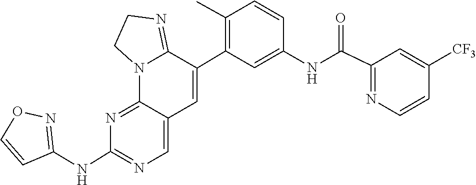 [SMILES: Cc1ccc(NC(=O)c2cc(C(F)(F)F)ccn2)cc1C1=Cc2cnc(Nc3ccon3)nc2N2CCN=C12] | N-(3-(2-(isoxazol-3- ylamino)-8,9- dihydroimidazo[1′2′:1,6] pyrido[2,3-d]pyrimidin- 6-yl)-4-methylphenyl)-4- (trifluoromethyl) picolinamide |
| 259 | 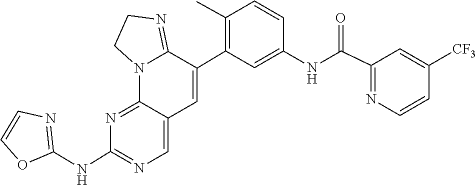 [SMILES: Cc1ccc(NC(=O)c2cc(C(F)(F)F)ccn2)cc1C1=Cc2cnc(Nc3ncco3)nc2N2CCN=C12] | N-(4-methyl-3-(2- (oxazol-2-ylamino)-8,9- dihydroimidazo[1′2′:1,6] pyrido[2,3-d]pyrimidin- 6-yl)phenyl)-4- (trifluoromethyl) picolinamide |
| 260 | 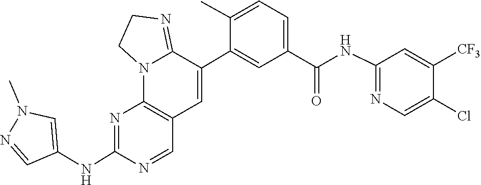 [SMILES: Cc1ccc(C(=O)Nc2cc(C(F)(F)F)c(Cl)cn2)cc1C1=Cc2cnc(Nc3cnn(C)c3)nc2N2CCN=C12] | N-(5-chloro-4- (trifluoromethyl)pyridin- 2-yl)-4-methyl-3-(2-((1- methyl-1H-pyrazol-4- yl)amino)-8,9- dihydroimidazo[1′2′:1,6] pyrido[2,3-d]pyrimidin- 6-yl)benzamide |
| 261 | 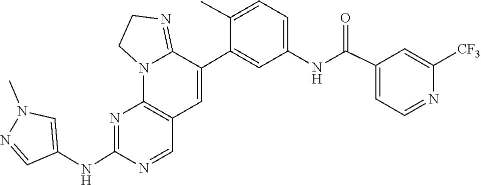 [SMILES: Cc1ccc(NC(=O)c2ccnc(C(F)(F)F)c2)cc1C1=Cc2cnc(Nc3cnn(C)c3)nc2N2CCN=C12] | N-(4-methyl-3-(2-((1- methyl-1H-pyrazol-4- yl)amino)-8,9- dihydroimidazo[1′2′:1,6] pyrido[2,3-d]pyrimidin- 6-yl)phenyl)-2- (trifluoromethyl) isonicotinamide |
| 262 | 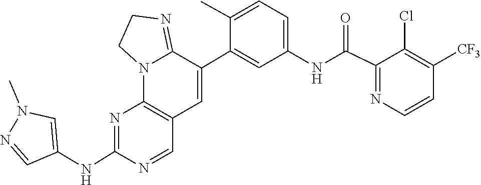 [SMILES: Cc1ccc(NC(=O)c2nccc(C(F)(F)F)c2Cl)cc1C1=Cc2cnc(Nc3cnn(C)c3)nc2N2CCN=C12] | 3-chloro-N-(4-methyl-3- (2-((1-methyl-1H- pyrazol-4-yl)amino)-8,9- dihydroimidazo[1′2′:1,6] pyrido[2,3-d]pyrimidin- 6-yl)phenyl)-4- (trifluoromethyl) picolinamide |
| 263 | 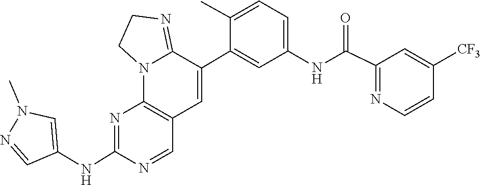 [SMILES: Cc1ccc(NC(=O)c2cc(C(F)(F)F)ccn2)cc1C1=Cc2cnc(Nc3cnn(C)c3)nc2N2CCN=C12] | 5-chloro-N-(4-methyl-3- (2-((1-methyl-1H- pyrazol-4-yl)amino)-8,9- dihydroimidazo[1′2′:1,6] pyrido[2,3-d]pyrimidin- 6-yl)phenyl)-4- (trifluoromethyl) picolinamide |
| 264 | 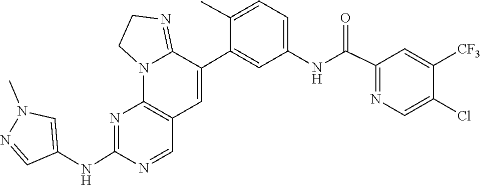 [SMILES: Cc1ccc(NC(=O)c2cc(C(F)(F)F)c(Cl)cn2)cc1C1=Cc2cnc(Nc3cnn(C)c3)nc2N2CCN=C12] | 4-chloro-N-(4-methyl-3- (2-((1-methyl-1H- pyrazol-4-yl)amino)-8,9- dihydroimidazo[1′2′:1,6] pyrido[2,3-d]pyrimidin- 6-yl)phenyl)-3- (trifluoromethyl) benzamide |
| 265 | 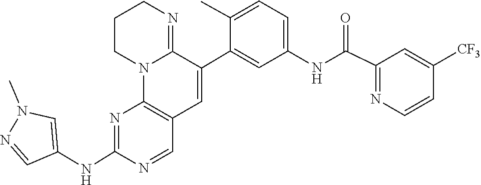 [SMILES: Cc1ccc(NC(=O)c2cc(C(F)(F)F)ccn2)cc1C1=Cc2cnc(Nc3cnn(C)c3)nc2N2CCCN=C12] | N-(4-methyl-3-(2-((1- methyl-1H-pyrazol-4- yl)amino)-9,10-dihydro- 8H-pyrido[1,6-a:2,3- d′]dipyrimidin-6- yl)phenyl)-4- (trifluoromethyl) picolinamide |
| 266 | 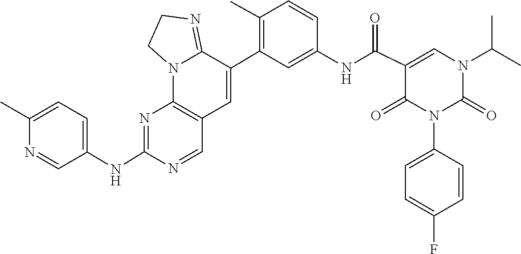 [SMILES: Cc1ccc(Nc2ncc3c(n2)N2CCN=C2C(c2cc(NC(=O)c4cn(C(C)C)c(=O)n(-c5ccc(F)cc5)c4=O)ccc2C)=C3)cn1] | 3-(4-fluorophenyl)-1- isopropyl-N-(4-methyl- 3-(2-((6-methylpyridin- 3-yl)amino)-8,9- dihydroimidazo[1′2′:1,6] pyrido[2,3-d]pyrimidin- 6-yl)phenyl)-2,4-dioxo- 1,2,3,4- tetrahydropyrimidine-5- carboxamide |
| 267 | 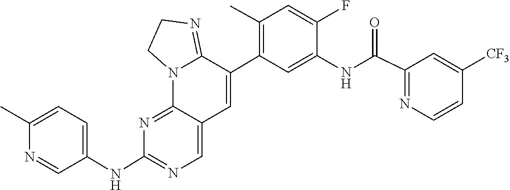 [SMILES: Cc1ccc(Nc2ncc3c(n2)N2CCN=C2C(c2cc(NC(=O)c4cc(C(F)(F)F)ccn4)c(F)cc2C)=C3)cn1] | N-(2-fluoro-4-methyl-5- (2-((6-methylpyridin-3- yl)amino)-8,9- dihydroimidazo[1′2′:1,6] pyrido[2,3-d]pyrimidin- 6-yl)phenyl)-4- (trifluoromethyl) picolinamide |
| 268 | 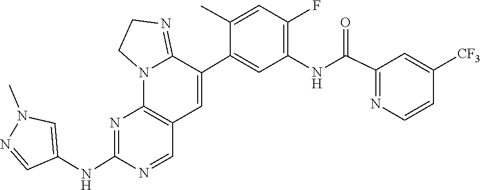 [SMILES: Cc1cc(F)c(NC(=O)c2cc(C(F)(F)F)ccn2)cc1C1=Cc2cnc(Nc3cnn(C)c3)nc2N2CCN=C12] | N-(2-fluoro-4-methyl-5- (2-((1-methyl-1H- pyrazol-4-yl)amino)-8,9- dihydroimidazo[1′2′:1,6] pyrido[2,3-d]pyrimidin- 6-yl)phenyl)-4- (trifluoromethyl) picolinamide |
| 269 | 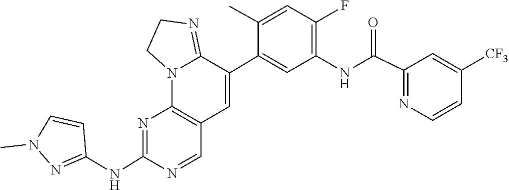 [SMILES: Cc1cc(F)c(NC(=O)c2cc(C(F)(F)F)ccn2)cc1C1=Cc2cnc(Nc3ccn(C)n3)nc2N2CCN=C12] | N-(2-fluoro-4-methyl-5- (2-((1-methyl-1H- pyrazol-3-yl)amino)-8,9- dihydroimidazo[1′2′:1,6] pyrido[2,3-d]pyrimidin- 6-yl)phenyl)-4- (trifluoromethyl) picolinamide |
| 270 | 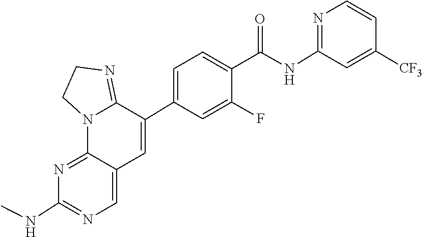 [SMILES: CNc1ncc2c(n1)N1CCN=C1C(c1ccc(C(=O)Nc3cc(C(F)(F)F)ccn3)c(F)c1)=C2] | 2-fluoro-4-(2- (methylamino)-8,9- dihydroimidazo[1′2′:1,6] pyrido[2,3-d]pyrimidin- 6-yl)-N-(4- (trifluoromethyl)pyridin- 2-yl)benzamide |
| 271 | 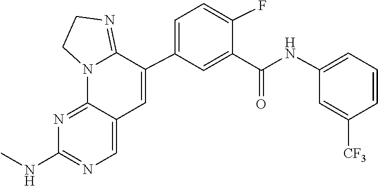 [SMILES: CNc1ncc2c(n1)N1CCN=C1C(c1ccc(F)c(C(=O)Nc3cccc(C(F)(F)F)c3)c1)=C2] | 2-fluoro-5-(2- (methylamino)-8,9- dihydroimidazo[1′2′:1,6] pyrido[2,3-d]pyrimidin- 6-yl)-N-(3- (trifluoromethyl)phenyl) benzamide |
| 272 | 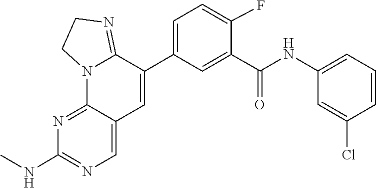 [SMILES: CNc1ncc2c(n1)N1CCN=C1C(c1ccc(F)c(C(=O)Nc3cccc(Cl)c3)c1)=C2] | N-(3-chlorophenyl)-2- fluoro-5-(2- (methylamino)-8,9- dihydroimidazo[1′2′:1,6] pyrido[2,3-d]pyrimidin- 6-yl)benzamide |
| 273 | 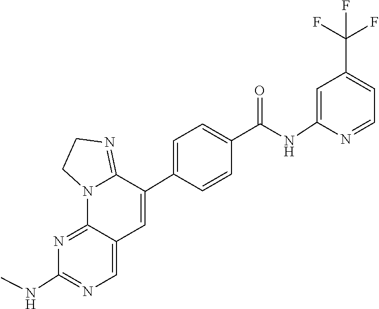 [SMILES: CNc1ncc2c(n1)N1CCN=C1C(c1ccc(C(=O)Nc3cc(C(F)(F)F)ccn3)cc1)=C2] | 4-(2-(methylamino)-8,9- dihydroimidazo[1′2′:1,6] pyrido[2,3-d]pyrimidin- 6-yl)-N-(4- (trifluoromethyl)pyridin- 2-yl)benzamide |
| 274 | 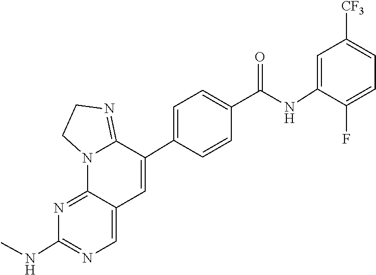 [SMILES: CNc1ncc2c(n1)N1CCN=C1C(c1ccc(C(=O)Nc3cc(C(F)(F)F)ccc3F)cc1)=C2] | N-(2-fluoro-5- (trifluoromethyl)phenyl)- 4-(2-(methylamino)-8,9- dihydroimidazo[1′2′:1,6] pyrido[2,3-d]pyrimidin- 6-yl)benzamide |
| 275 | 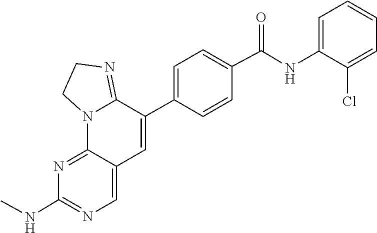 [SMILES: CNc1ncc2c(n1)N1CCN=C1C(c1ccc(C(=O)Nc3ccccc3Cl)cc1)=C2] | N-(2-chlorophenyl)-4-(2- (methylamino)-8,9- dihydroimidazo[1′2′:1,6] pyrido[2,3-d]pyrimidin- 6-yl)benzamide |
| 276 | 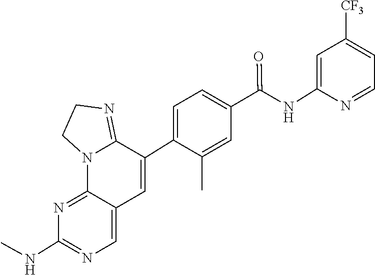 [SMILES: CNc1ncc2c(n1)N1CCN=C1C(c1ccc(C(=O)Nc3cc(C(F)(F)F)ccn3)cc1C)=C2] | 3-methyl-4-(2- (methylamino)-8,9- dihydroimidazo[1′2′:1,6] pyrido[2,3-d]pyrimidin- 6-yl)-N-(4- (trifluoromethyl)pyridin- 2-yl)benzamide |
| 277 | 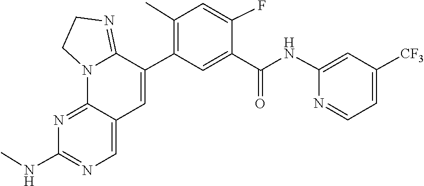 [SMILES: CNc1ncc2c(n1)N1CCN=C1C(c1cc(C(=O)Nc3cc(C(F)(F)F)ccn3)c(F)cc1C)=C2] | 2-fluoro-4-methyl-5-(2- (methylamino)-8,9- dihydroimidazo[1′2′:1,6] pyrido[2,3-d]pyrimidin- 6-yl)-N-(4- (trifluoromethyl)pyridin- 2-yl)benzamide |
| 278 | 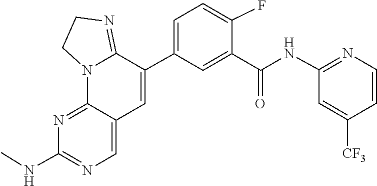 [SMILES: CNc1ncc2c(n1)N1CCN=C1C(c1ccc(F)c(C(=O)Nc3cc(C(F)(F)F)ccn3)c1)=C2] | 2-fluoro-5-(2- (methylamino)-8,9- dihydroimidazo[1′2′:1,6] pyrido[2,3-d]pyrimidin- 6-yl)-N-(4- (trifluoromethyl)pyridin- 2-yl)benzamide |
| 279 | 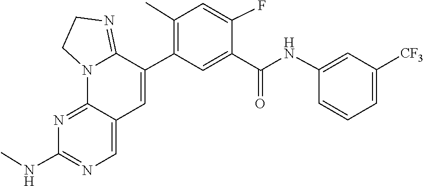 [SMILES: CNc1ncc2c(n1)N1CCN=C1C(c1cc(C(=O)Nc3cccc(C(F)(F)F)c3)c(F)cc1C)=C2] | 2-fluoro-4-methyl-5-(2- (methylamino)-8,9- dihydroimidazo[1′2′:1,6] pyrido[2,3-d]pyrimidin- 6-yl)-N-(3- (trifluoromethyl)phenyl) benzamide |
| 280 | 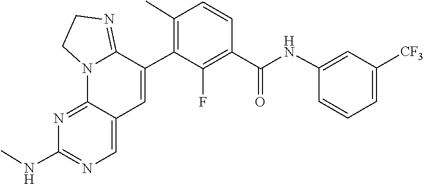 [SMILES: CNc1ncc2c(n1)N1CCN=C1C(c1c(C)ccc(C(=O)Nc3cccc(C(F)(F)F)c3)c1F)=C2] | 2-fluoro-4-methyl-3-(2- (methylamino)-8,9- dihydroimidazo[1′2′:1,6] pyrido[2,3-d]pyrimidin- 6-yl)-N-(3- (trifluoromethyl)phenyl) benzamide |
| 281 | 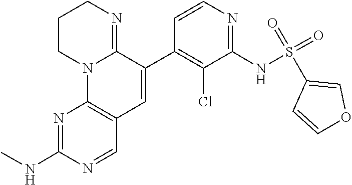 [SMILES: CNc1ncc2c(n1)N1CCCN=C1C(c1ccnc(NS(=O)(=O)c3ccoc3)c1Cl)=C2] | N-(3-chloro-4-(2- (methylamino)-9,10- dihydro-8H-pyrido[1,6- a:2,3-d′]dipyrimidin-6- yl)pyridin-2-yl)furan-3- sulfonamide |
| 282 | 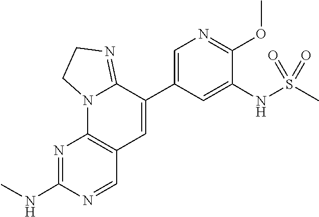 | N-(2-methoxy-5-(2- (methylamino)-8,9- dihydroimidazo[1′2′:1,6] pyrido[2,3-d]pyrimidin- 6-yl)pyridin-3- yl)methanesulfonamide |
| 283 | 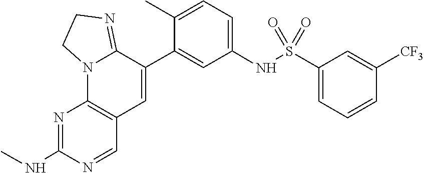 [SMILES: CNc1ncc2c(n1)N1CCN=C1C(c1cc(NS(=O)(=O)c3cccc(C(F)(F)F)c3)ccc1C)=C2] | N-(4-methyl-3-(2- (methylamino)-8,9- dihydroimidazo[1′2′:1,6] pyrido[2,3-d]pyrimidin- 6-yl)phenyl)-3- (trifluoromethyl) benzenesulfonamide |
| 284 | 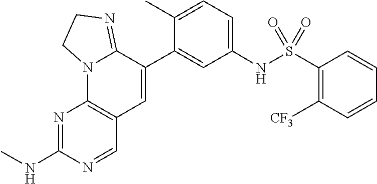 [SMILES: CNc1ncc2c(n1)N1CCN=C1C(c1cc(NS(=O)(=O)c3ccccc3C(F)(F)F)ccc1C)=C2] | N-(4-methyl-3-(2- (methylamino)-8,9- dihydroimidazo[1′2′:1,6] pyrido[2,3-d]pyrimidin- 6-yl)phenyl)-2- (trifluoromethyl) benzenesulfonamide |
| 285 | 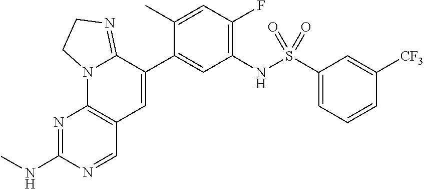 [SMILES: CNc1ncc2c(n1)N1CCN=C1C(c1cc(NS(=O)(=O)c3cccc(C(F)(F)F)c3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-3- (trifluoromethyl)benzene sulfonamide |
| 286 | 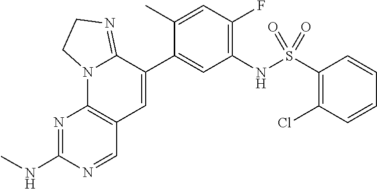 [SMILES: CNc1ncc2c(n1)N1CCN=C1C(c1cc(NS(=O)(=O)c3ccccc3Cl)c(F)cc1C)=C2] | 2-chloro-N-(2-fluoro-4- methyl-5-(2- (methylamino)-8,9- dihydroimidazo[1′2′:1,6] pyrido[2,3-d]pyrimidin- 6- yl)phenyl) benzenesulfonamide |
| 287 | 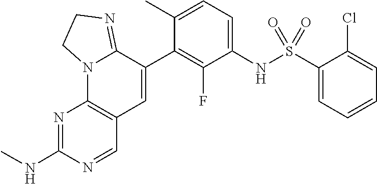 [SMILES: CNc1ncc2c(n1)N1CCN=C1C(c1c(C)ccc(NS(=O)(=O)c3ccccc3Cl)c1F)=C2] | 2-chloro-N-(2-fluoro-4- methyl-3-(2- (methylamino)-8,9- dihydroimidazo[1′2′:1,6] pyrido[2,3-d]pyrimidin- 6- yl)phenyl) benzenesulfonamide |
| 288 | 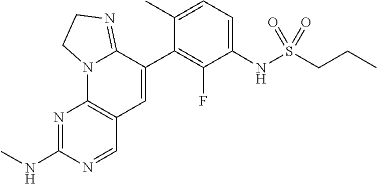 [SMILES: CCCS(=O)(=O)Nc1ccc(C)c(C2=Cc3cnc(NC)nc3N3CCN=C23)c1F] | N-(2-fluoro-4-methyl-3- (2-(methylamino)-8,9- dihydroimidazo[1′2′:1,6] pyrido[2,3-d]pyrimidin- 6-yl)phenyl)propane-1- sulfonamide |
| 289 | 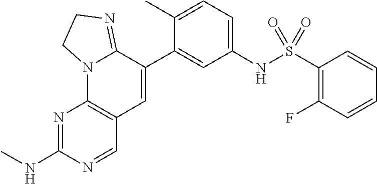 [SMILES: CNc1ncc2c(n1)N1CCN=C1C(c1cc(NS(=O)(=O)c3ccccc3F)ccc1C)=C2] | 2-fluoro-N-(4-methyl-3- (2-(methylamino)-8,9- dihydroimidazo[1′2′:1,6] pyrido[2,3-d]pyrimidin- 6- yl)phenyl) benzenesulfonamide |
| 290 | 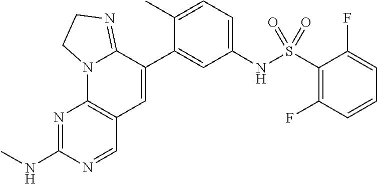 [SMILES: CNc1ncc2c(n1)N1CCN=C1C(c1cc(NS(=O)(=O)c3c(F)cccc3F)ccc1C)=C2] | 2,6-difluoro-N-(4- methyl-3-(2- (methylamino)-8,9- dihydroimidazo[1′2′:1,6] pyrido[2,3-d]pyrimidin- 6- yl)phenyl) benzenesulfonamide |
| 291 | 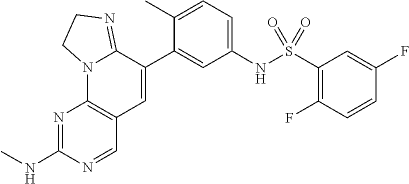 [SMILES: CNc1ncc2c(n1)N1CCN=C1C(c1cc(NS(=O)(=O)c3cc(F)ccc3F)ccc1C)=C2] | 2,5-difluoro-N-(4- methy1-3-(2- (methylamino)-8,9- dihydroimidazo[1′2′:1,6] pyrido[2,3-d]pyrimidin- 6- yl)phenyl) benzenesulfonamide |
| 292 | 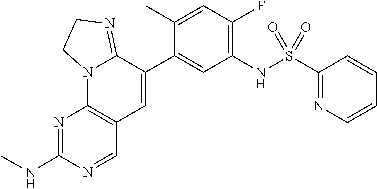 [SMILES: CNc1ncc2c(n1)N1CCN=C1C(c1cc(NS(=O)(=O)c3ccccn3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)pyridine-2- sulfonamide |
| 293 | 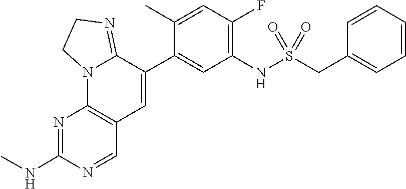 [SMILES: CNc1ncc2c(n1)N1CCN=C1C(c1cc(NS(=O)(=O)Cc3ccccc3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-1- phenylmethane- sulfonamide |
| 294 | 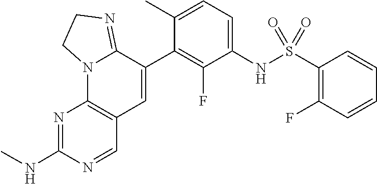 [SMILES: CNc1ncc2c(n1)N1CCN=C1C(c1c(C)ccc(NS(=O)(=O)c3ccccc3F)c1F)=C2] | 2-fluoro-N-(2-fluoro-4- methyl-3-(2- (methylamino)-8,9- dihydroimidazo[1′2′:1,6] pyrido[2,3-d]pyrimidin- 6- yl)phenyl) benzenesulfonamide |
| 295 | 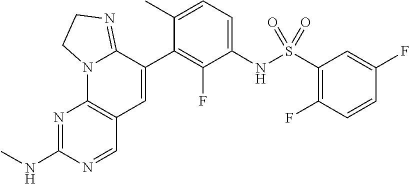 [SMILES: CNc1ncc2c(n1)N1CCN=C1C(c1c(C)ccc(NS(=O)(=O)c3cc(F)ccc3F)c1F)=C2] | 2,5-difluoro-N-(2-fluoro- 4-methyl-3-(2- (methylamino)-8,9- dihydroimidazo[1′2′:1,6] pyrido[2,3-d]pyrimidin- 6- yl)phenyl) benzenesulfonamide |
| 296 | 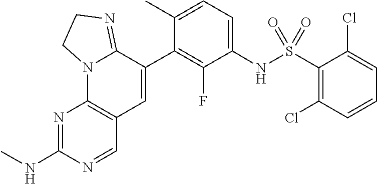 [SMILES: CNc1ncc2c(n1)N1CCN=C1C(c1c(C)ccc(NS(=O)(=O)c3c(Cl)cccc3Cl)c1F)=C2] | 2,6-dichloro-N-(2-fluoro- 4-methyl-3-(2- (methylamino)-8,9- dihydroimidazo[1′2′:1,6] pyrido[2,3-d]pyrimidin- 6- yl)phenyl) benzenesulfonamide |
| 297 | 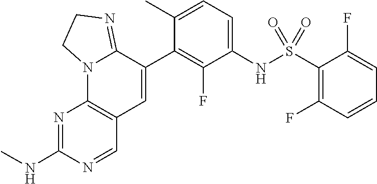 [SMILES: CNc1ncc2c(n1)N1CCN=C1C(c1c(C)ccc(NS(=O)(=O)c3c(F)cccc3F)c1F)=C2] | 2,6-difluoro-N-(2-fluoro- 4-methyl-3-(2- (methylamino)-8,9- dihydroimidazo[1′2′:1,6] pyrido[2,3-d]pyrimidin- 6- yl)phenyl) benzenesulfonamide |
| 298 | 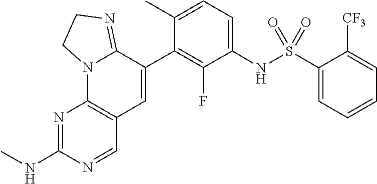 [SMILES: CNc1ncc2c(n1)N1CCN=C1C(c1c(C)ccc(NS(=O)(=O)c3ccccc3C(F)(F)F)c1F)=C2] | N-(2-fluoro-4-methyl-3- (2-(methylamino)-8,9- dihydroimidazo[1′2′:1,6] pyrido[2,3-d]pyrimidin- 6-yl)phenyl)-2- (trifluoromethyl) benzenesulfonamide |
| 299 | 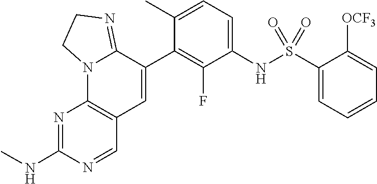 [SMILES: CNc1ncc2c(n1)N1CCN=C1C(c1c(C)ccc(NS(=O)(=O)c3ccccc3OC(F)(F)F)c1F)=C2] | N-(2-fluoro-4-methyl-3- (2-(methylamino)-8,9- dihydroimidazo[1′2′:1,6] pyrido[2,3-d]pyrimidin- 6-yl)phenyl)-2- (trifluoromethoxy) benzenesulfonamide |
| 300 | 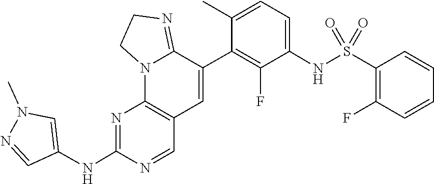 [SMILES: Cc1ccc(NS(=O)(=O)c2ccccc2F)c(F)c1C1=Cc2cnc(Nc3cnn(C)c3)nc2N2CCN=C12] | 2-fluoro-N-(2-fluoro-4- methyl-3-(2-((1-methyl- 1H-pyrazol-4-yl)amino)- 8,9- dihydroimidazo[1′2′:1,6] pyrido[2,3-d]pyrimidin- 6- yl)phenyl) benzenesulfonamide |
| 301 | 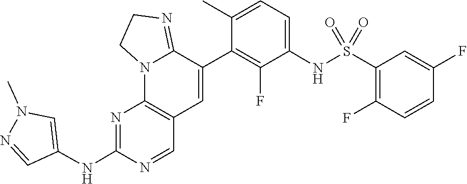 [SMILES: Cc1ccc(NS(=O)(=O)c2cc(F)ccc2F)c(F)c1C1=Cc2cnc(Nc3cnn(C)c3)nc2N2CCN=C12] | 2,5-difluoro-N-(2-fluoro- 4-methyl-3-(2-((1- methyl-1H-pyrazol-4- yl)amino)-8,9- dihydroimidazo[1′2′:1,6] pyrido[2,3-d]pyrimidin- 6- yl)phenyl) benzenesulfonamide |
| 302 | 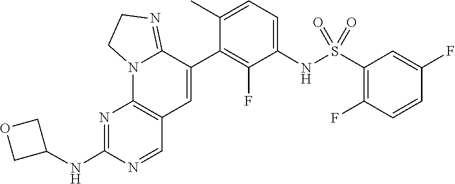 [SMILES: Cc1ccc(NS(=O)(=O)c2cc(F)ccc2F)c(F)c1C1=Cc2cnc(NC3COC3)nc2N2CCN=C12] | 2,5-difluoro-N-(2-fluoro- 4-methyl-3-(2-(oxetan-3- ylamino)-8,9- dihydroimidazo[1′2′:1,6] pyrido[2,3-d]pyrimidin- 6- yl)phenyl) benzenesulfonamide |
| 303 | 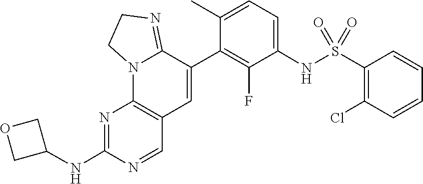 [SMILES: Cc1ccc(NS(=O)(=O)c2ccccc2Cl)c(F)c1C1=Cc2cnc(NC3COC3)nc2N2CCN=C12] | 2-chloro-N-(2-fluoro-4- methyl-3-(2-(oxetan-3- ylamino)-8,9- dihydroimidazo[1′2′:1,6] pyrido[2,3-d]pyrimidin- 6- yl)phenyl) benzenesulfonamide |
| 304 | 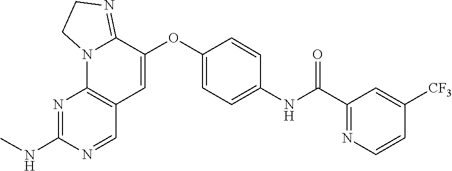 [SMILES: CNc1ncc2c(n1)N1CCN=C1C(Oc1ccc(NC(=O)c3cc(C(F)(F)F)ccn3)cc1)=C2] | N-(4-((2-(methylamino)- 8,9- dihydroimidazo[1′2′:1,6] pyrido[2,3-d]pyrimidin- 6-yl)oxy)phenyl)-4- (trifluoromethyl) picolinamide |
| 305 | 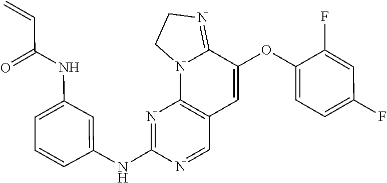 [SMILES: C=CC(=O)Nc1cccc(Nc2ncc3c(n2)N2CCN=C2C(Oc2ccc(F)cc2F)=C3)c1] | N-(3-((6-(2,4- difluorophenoxy)-8,9- dihydroimidazo[1′2′:1,6] pyrido[2,3-d]pyrimidin- 2- yl)amino)phenyl) acrylamide |
| 306 | 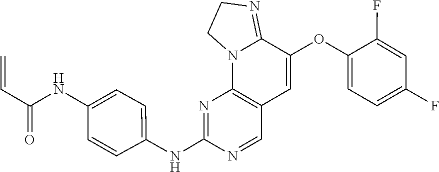 [SMILES: C=CC(=O)Nc1ccc(Nc2ncc3c(n2)N2CCN=C2C(Oc2ccc(F)cc2F)=C3)cc1] | N-(4-((6-(2,4- difluorophenoxy)-8,9- dihydroimidazo[1′2′:1,6] pyrido[2,3-d]pyrimidin- 2- yl)amino)phenyl) acrylamide |
| 307 | 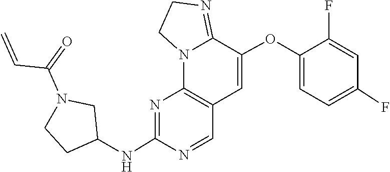 [SMILES: C=CC(=O)N1CCC(Nc2ncc3c(n2)N2CCN=C2C(Oc2ccc(F)cc2F)=C3)C1] | 1-(3-((6-(2,4- difluorophenoxy)-8,9- dihydroimidazo[1′2′:1,6] pyrido[2,3-d]pyrimidin- 2-yl)amino)pyrrolidin-1- yl)prop-2-en-1-one |
| 308 | 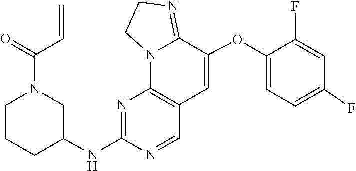 [SMILES: C=CC(=O)N1CCCC(Nc2ncc3c(n2)N2CCN=C2C(Oc2ccc(F)cc2F)=C3)C1] | 1-(3-((6-(2,4- difluorophenoxy)-8,9- dihydroimidazo[1′2′:1,6] pyrido[2,3-d]pyrimidin- 2-yl)amino)piperidin-1- yl)prop-2-en-1-one |
| 309 | 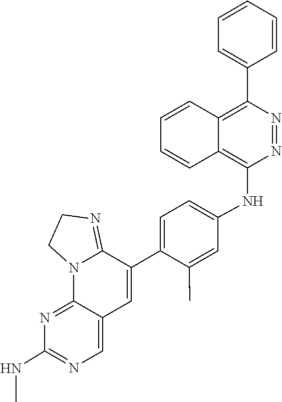 [SMILES: CNc1ncc2c(n1)N1CCN=C1C(c1ccc(Nc3nnc(-c4ccccc4)c4ccccc34)cc1C)=C2] | N-methyl-6-(2-methyl-4- ((4-phenylphthalazin-1- yl)amino)phenyl)-8,9- dihydroimidazo[1′2′:1,6] pyrido[2,3-d]pyrimidin- 2-amine |
| 310 | 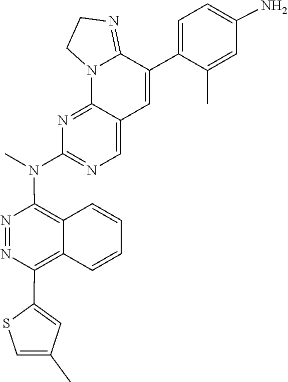 [SMILES: Cc1csc(-c2nnc(N(C)c3ncc4c(n3)N3CCN=C3C(c3ccc(N)cc3C)=C4)c3ccccc23)c1] | 6-(4-amino-2- methylpheny1)-N- methyl-N-(4-(4- methylthiophen-2- yl)phthalazin-1-yl)-8,9- dihydroimidazo[1′2′:1,6] pyrido[2,3-d]pyrimidin- 2-amine |
| 311 | 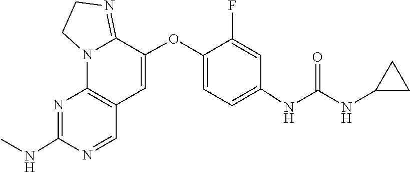 [SMILES: CNc1ncc2c(n1)N1CCN=C1C(Oc1ccc(NC(=O)NC3CC3)cc1F)=C2] | 1-cyclopropyl-3-(3- fluoro-4-((2- (methylamino)-8,9- dihydroimidazo[1′2′:1,6] pyrido[2,3-d]pyrimidin- 6-yl)oxy)phenyl)urea |
| 312 | 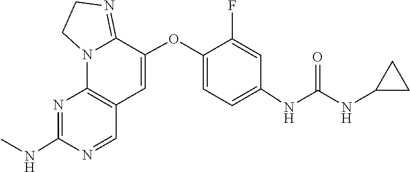 [SMILES: CNc1ncc2c(n1)N1CCN=C1C(Oc1ccc(NC(=O)NC3CC3)cc1F)=C2] | 1-cyclopropyl-3-(4-((2- (methylamino)-8,9- dihydroimidazo[1′2′:1,6] pyrido[2,3-d]pyrimidin- 6-yl)oxy)phenyl)urea |
| 313 | 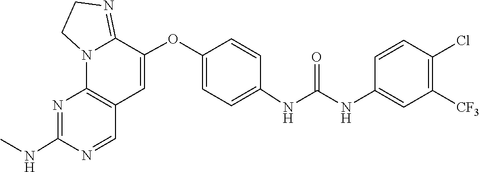 [SMILES: CNc1ncc2c(n1)N1CCN=C1C(Oc1ccc(NC(=O)Nc3ccc(Cl)c(C(F)(F)F)c3)cc1)=C2] | 1-(4-chloro-3- (trifluoromethyl)phenyl)- 3-(4-((2-(methylamino)- 8,9- dihydroimidazo[1′2′:1,6] pyrido[2,3-d]pyrimidin- 6-yl)oxy)phenyl)urea |
| 314 | 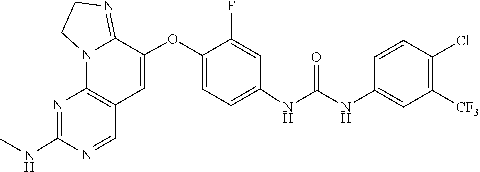 [SMILES: CNc1ncc2c(n1)N1CCN=C1C(Oc1ccc(NC(=O)Nc3ccc(Cl)c(C(F)(F)F)c3)cc1F)=C2] | 1-(4-chloro-3- (trifluoromethyl)phenyl)- 3-(3-fluoro-4-((2- (methylamino)-8,9- dihydroimidazo[1′2′:1,6] pyrido[2,3-d]pyrimidin- 6-yl)oxy)phenyl)urea |
| 315 | 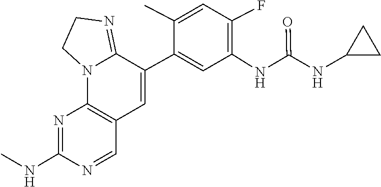 [SMILES: CNc1ncc2c(n1)N1CCN=C1C(c1cc(NC(=O)NC3CC3)c(F)cc1C)=C2] | 1-cyclopropyl-3-(2- fluoro-4-methyl-5-(2- (methylamino)-8,9- dihydroimidazo[1′2′:1,6] pyrido[2,3-d]pyrimidin- 6-yl)phenyl)urea |
| 316 | 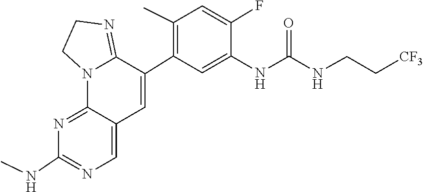 [SMILES: CNc1ncc2c(n1)N1CCN=C1C(c1cc(NC(=O)NCCC(F)(F)F)c(F)cc1C)=C2] | 1-(2-fluoro-4-methyl-5- (2-(methylamino)-8,9- dihydroimidazo[1′2′:1,6] pyrido[2,3-d]pyrimidin- 6-yl)phenyl)-3-(3,3,3- trifluoropropyl)urea |
| 317 | 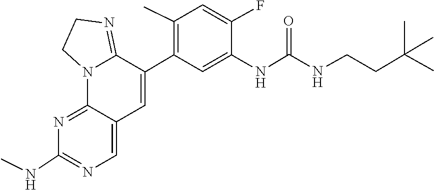 [SMILES: CNc1ncc2c(n1)N1CCN=C1C(c1cc(NC(=O)NCCC(C)(C)C)c(F)cc1C)=C2] | 1-(3,3-dimethylbutyl)-3- (2-fluoro-4-methyl-5-(2- (methylamino)-8,9- dihydroimidazo[1′2′:1,6] pyrido[2,3-d]pyrimidin- 6-yl)phenyl)urea |
| 318 | 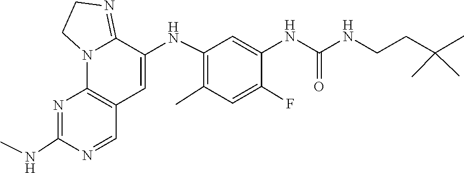 [SMILES: CNc1ncc2c(n1)N1CCN=C1C(Nc1cc(NC(=O)NCCC(C)(C)C)c(F)cc1C)=C2] | 1-(3,3-dimethylbutyl)-3- (2-fluoro-4-methyl-5-((2- (methylamino)-8,9- dihydroimidazo[1′2′:1,6] pyrido[2,3-d]pyrimidin- 6-yl)amino)phenyl)urea |
| 319 | 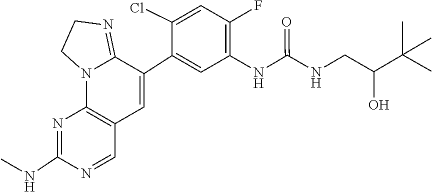 [SMILES: CNc1ncc2c(n1)N1CCN=C1C(c1cc(NC(=O)NCC(O)C(C)(C)C)c(F)cc1Cl)=C2] | 1-(4-chloro-2-fluoro-5- (2-(methylamino)-8,9- dihydroimidazo[1′2′:1,6] pyrido[2,3-d]pyrimidin- 6-yl)phenyl)-3-(2- hydroxy-3,3- dimethylbutyl)urea |
| 320 | 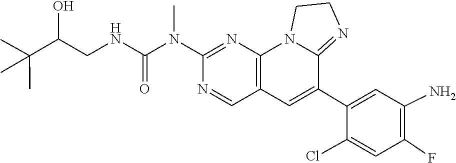 [SMILES: CN(C(=O)NCC(O)C(C)(C)C)c1ncc2c(n1)N1CCN=C1C(c1cc(N)c(F)cc1Cl)=C2] | 1-(6-(5-amino-2-chloro- 4-fluorophenyl)-8,9- dihydroimidazo[1′2′:1,6] pyrido[2,3-d]pyrimidin- 2-yl)-3-(2-hydroxy-3,3- dimethylbutyl)-1- methylurea |
| 321 | 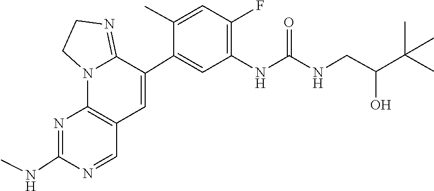 [SMILES: CNc1ncc2c(n1)N1CCN=C1C(c1cc(NC(=O)NCC(O)C(C)(C)C)c(F)cc1C)=C2] | 1-(2-fluoro-4-methyl-5- (2-(methylamino)-8,9- dihydroimidazo[1′2′:1,6] pyrido[2,3-d]pyrimidin- 6-yl)phenyl)-3-(2- hydroxy-3,3- dimethylbutyl)urea |
| 322 | 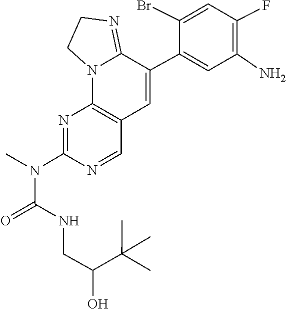 [SMILES: CN(C(=O)NCC(O)C(C)(C)C)c1ncc2c(n1)N1CCN=C1C(c1cc(N)c(F)cc1Br)=C2] | 1-(6-(5-amino-2-bromo- 4-fluorophenyl)-8,9- dihydroimidazo[1′2′:1,6] pyrido[2,3-d]pyrimidin- 2-yl)-3-(2-hydroxy-3,3- dimethylbutyl)-1- methylurea |
| 323 | 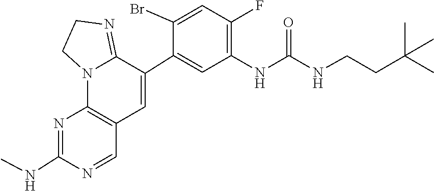 [SMILES: CNc1ncc2c(n1)N1CCN=C1C(c1cc(NC(=O)NCCC(C)(C)C)c(F)cc1Br)=C2] | 1-(4-bromo-2-fluoro-5- (2-(methylamino)-8,9- dihydroimidazo[1′2′:1,6] pyrido[2,3-d]pyrimidin- 6-yl)phenyl)-3-(3,3- dimethylbutyl)urea |
| 324 | 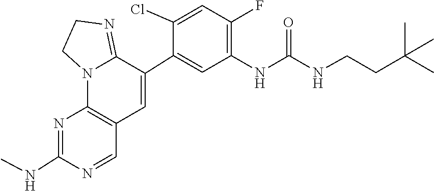 [SMILES: CNc1ncc2c(n1)N1CCN=C1C(c1cc(NC(=O)NCCC(C)(C)C)c(F)cc1Cl)=C2] | 1-(4-chloro-2-fluoro-5- (2-(methylamino)-8,9- dihydroimidazo[1′2′:1,6] pyrido[2,3-d]pyrimidin- 6-yl)phenyl)-3-(3,3- dimethylbutyl)urea |
| 325 | 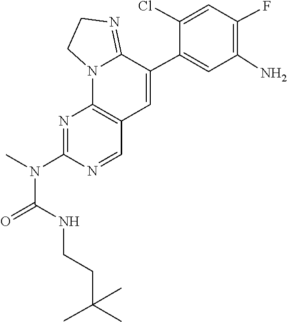 [SMILES: CN(C(=O)NCCC(C)(C)C)c1ncc2c(n1)N1CCN=C1C(c1cc(N)c(F)cc1Cl)=C2] | 1-(6-(5-amino-2-chloro- 4-fluorophenyl)-8,9- dihydroimidazo[1′2′:1,6] pyrido[2,3-d]pyrimidin- 2-yl)-3-(3,3- dimethylbutyl)-1- methylurea |
| 326 | 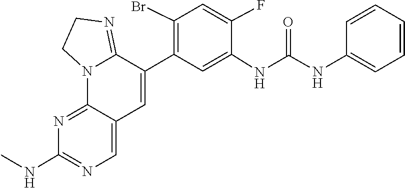 [SMILES: CNc1ncc2c(n1)N1CCN=C1C(c1cc(NC(=O)Nc3ccccc3)c(F)cc1Br)=C2] | 1-(4-bromo-2-fluoro-5- (2-(methylamino)-8,9- dihydroimidazo[1′2′:1,6] pyrido[2,3-d]pyrimidin- 6-yl)phenyl)-3- phenylurea |
| 327 | 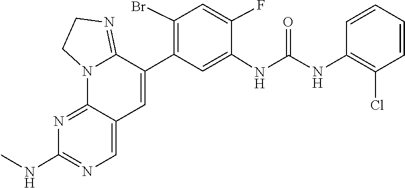 [SMILES: CNc1ncc2c(n1)N1CCN=C1C(c1cc(NC(=O)Nc3ccccc3Cl)c(F)cc1Br)=C2] | 1-(4-bromo-2-fluoro-5- (2-(methylamino)-8,9- dihydroimidazo[1′2′:1,6] pyrido[2,3-d]pyrimidin- 6-yl)phenyl)-3-(2- chlorophenyl)urea |
| 328 | 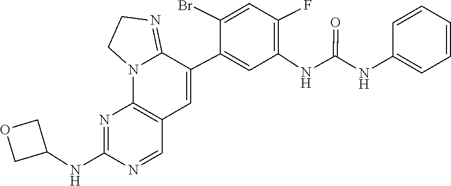 [SMILES: O=C(Nc1ccccc1)Nc1cc(C2=Cc3cnc(NC4COC4)nc3N3CCN=C23)c(Br)cc1F] | 1-(4-bromo-2-fluoro-5- (2-(oxetan-3-ylamino)- 8,9- dihydroimidazo[1′2′:1,6] pyrido[2,3-d]pyrimidin- 6-yl)phenyl)-3- phenylurea |
| 329 | 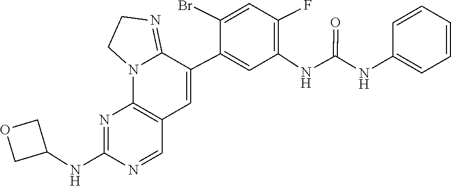 [SMILES: O=C(Nc1ccccc1)Nc1cc(C2=Cc3cnc(NC4COC4)nc3N3CCN=C23)c(Br)cc1F] | 1-(4-bromo-2-fluoro-5- (2-(oxetan-3-ylamino)- 8,9- dihydroimidazo[1′2′:1,6] pyrido[2,3-d]pyrimidin- 6-yl)phenyl)-3-(2- chlorophenyl)urea |
| 330 | 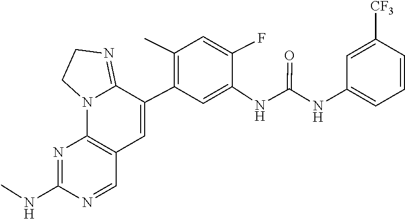 [SMILES: CNc1ncc2c(n1)N1CCN=C1C(c1cc(NC(=O)Nc3cccc(C(F)(F)F)c3)c(F)cc1C)=C2] | 1-(2-fluoro-4-methyl-5- (2-(methylamino)-8,9- dihydroimidazo[1′2′:1,6] pyrido[2,3-d]pyrimidin- 6-yl)phenyl)-3-(3- (trifluoromethyl)phenyl) urea |
| 331 | 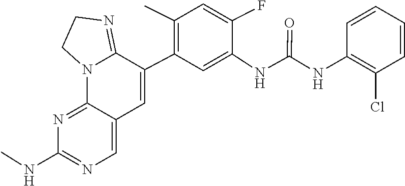 [SMILES: CNc1ncc2c(n1)N1CCN=C1C(c1cc(NC(=O)Nc3ccccc3Cl)c(F)cc1C)=C2] | 1-(2-chlorophenyl)-3-(2- fluoro-4-methyl-5-(2- (methylamino)-8,9- dihydroimidazo[1′2′:1,6] pyrido[2,3-d]pyrimidin- 6-yl)phenyl)urea |
| 332 | 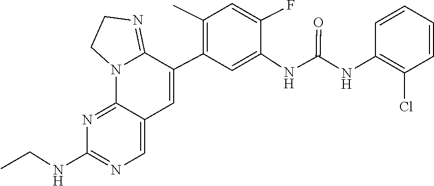 [SMILES: CCNc1ncc2c(n1)N1CCN=C1C(c1cc(NC(=O)Nc3ccccc3Cl)c(F)cc1C)=C2] | 1-(2-chlorophenyl)-3-(5- (2-(ethylamino)-8,9- dihydroimidazo[1′2′:1,6] pyrido[2,3-d]pyrimidin- 6-yl)-2-fluoro-4- methylphenyl)urea |
| 333 | 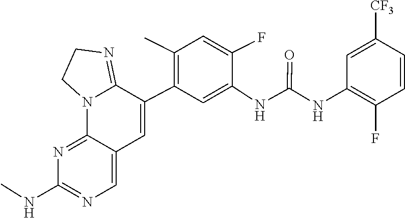 [SMILES: CNc1ncc2c(n1)N1CCN=C1C(c1cc(NC(=O)Nc3cc(C(F)(F)F)ccc3F)c(F)cc1C)=C2] | 1-(2-fluoro-4-methyl-5- (2-(methylamino)-8,9- dihydroimidazo[1′2′:1,6] pyrido[2,3-d]pyrimidin- 6-yl)phenyl)-3-(2-fluoro- 5- (trifluoromethyl)phenyl) urea |
| 334 | 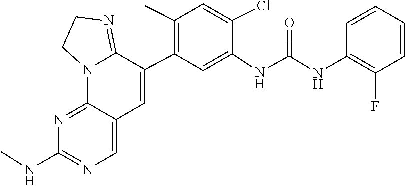 [SMILES: CNc1ncc2c(n1)N1CCN=C1C(c1cc(NC(=O)Nc3ccccc3F)c(Cl)cc1C)=C2] | 1-(2-chloro-4-methyl-5- (2-(methylamino)-8,9- dihydroimidazo[1′2′:1,6] pyrido[2,3-d]pyrimidin- 6-yl)phenyl)-3-(2- fluorophenyl)urea |
| 335 | 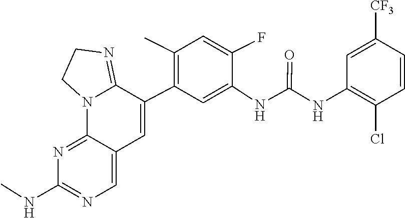 [SMILES: CNc1ncc2c(n1)N1CCN=C1C(c1cc(NC(=O)Nc3cc(C(F)(F)F)ccc3Cl)c(F)cc1C)=C2] | 1-(2-chloro-5- (trifluoromethyl)phenyl)- 3-(2-fluoro-4-methyl-5- (2-(methylamino)-8,9- dihydroimidazo[1′2′:1,6] pyrido[2,3-d]pyrimidin- 6-yl)phenyl)urea |
| 336 | 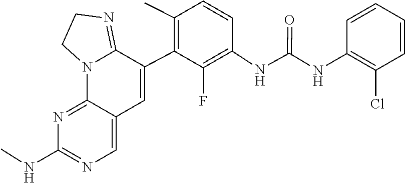 [SMILES: CNc1ncc2c(n1)N1CCN=C1C(c1c(C)ccc(NC(=O)Nc3ccccc3Cl)c1F)=C2] | 1-(2-chlorophenyl)-3-(2- fluoro-4-methyl-3-(2- (methylamino)-8,9- dihydroimidazo[1′2′:1,6] pyrido[2,3-d]pyrimidin- 6-yl)phenyl)urea |
| 337 | 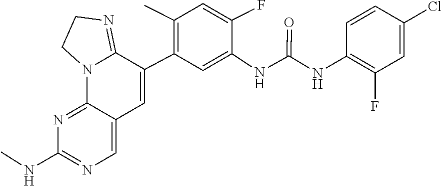 [SMILES: CNc1ncc2c(n1)N1CCN=C1C(c1cc(NC(=O)Nc3ccc(Cl)cc3F)c(F)cc1C)=C2] | 1-(4-chloro-2- fluorophenyl)-3-(2- fluoro-4-methyl-5-(2- (methylamino)-8,9- dihydroimidazo[1′2′:1,6] pyrido[2,3-d]pyrimidin- 6-yl)phenyl)urea |
| 338 | 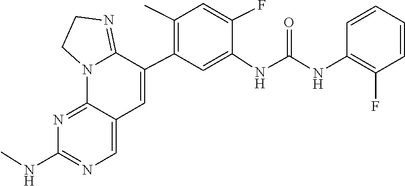 [SMILES: CNc1ncc2c(n1)N1CCN=C1C(c1cc(NC(=O)Nc3ccccc3F)c(F)cc1C)=C2] | 1-(2-fluoro-4-methyl-5- (2-(methylamino)-8,9- dihydroimidazo[1′2′:1,6] pyrido[2,3-d]pyrimidin- 6-yl)phenyl)-3-(2- fluorophenyl)urea |
| 339 | 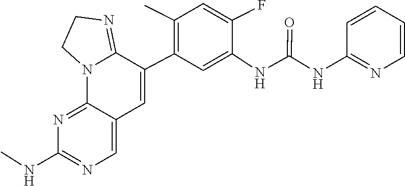 [SMILES: CNc1ncc2c(n1)N1CCN=C1C(c1cc(NC(=O)Nc3ccccn3)c(F)cc1C)=C2] | 1-(2-fluoro-4-methyl-5- (2-(methylamino)-8,9- dihydroimidazo[1′2′:1,6] pyrido[2,3-d]pyrimidin- 6-yl)phenyl)-3-(pyridin- 2-yl)urea |
| 340 | 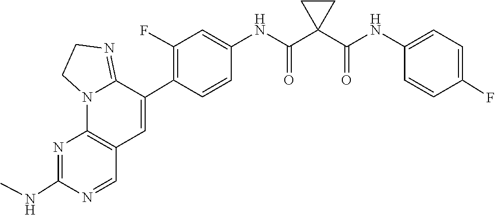 [SMILES: CNc1ncc2c(n1)N1CCN=C1C(c1ccc(NC(=O)C3(C(=O)Nc4ccc(F)cc4)CC3)cc1F)=C2] | N-(3-fluoro-4-(2- (methylamino)-8,9- dihydroimidazo[1′2′:1,6] pyrido[2,3-d]pyrimidin- 6-yl)phenyl)-N-(4- fluorophenyl) cyclopropane-1,1- dicarboxamide |
| 341 | 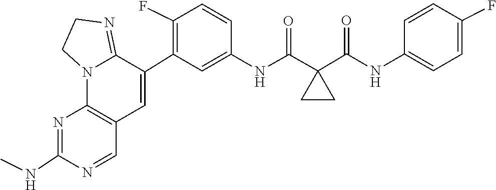 [SMILES: CNc1ncc2c(n1)N1CCN=C1C(c1cc(NC(=O)C3(C(=O)Nc4ccc(F)cc4)CC3)ccc1F)=C2] | N-(4-fluoro-3-(2- (methylamino)-8,9- dihydroimidazo[1′2′:1,6] pyrido[2,3-d]pyrimidin- 6-yl)phenyl)-N-(4- fluorophenyl) cyclopropane-1,1- dicarboxamide |
| 342 | 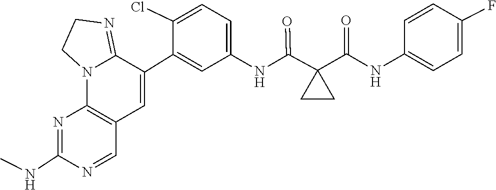 [SMILES: CNc1ncc2c(n1)N1CCN=C1C(c1cc(NC(=O)C3(C(=O)Nc4ccc(F)cc4)CC3)ccc1Cl)=C2] | N-(4-chloro-3-(2- (methylamino)-8,9- dihydroimidazo[1′2′:1,6] pyrido[2,3-d]pyrimidin- 6-yl)phenyl)-N-(4- fluorophenyl) cyclopropane-1,1- dicarboxamide |
| 343 | 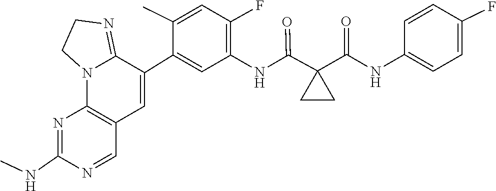 [SMILES: CNc1ncc2c(n1)N1CCN=C1C(c1cc(NC(=O)C3(C(=O)Nc4ccc(F)cc4)CC3)c(F)cc1C)=C2] | N-(2-fluoro-4-methyl-5- (2-(methylamino)-8,9- dihydroimidazo[1′2′:1,6] pyrido[2,3-d]pyrimidin- 6-yl)phenyl)-N-(4- fluorophenyl) cyclopropane-1,1- dicarboxamide |
| 344 | 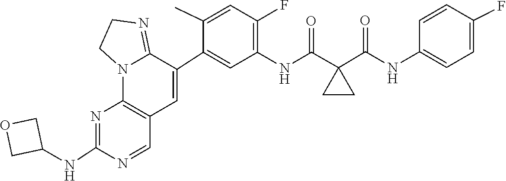 [SMILES: Cc1cc(F)c(NC(=O)C2(C(=O)Nc3ccc(F)cc3)CC2)cc1C1=Cc2cnc(NC3COC3)nc2N2CCN=C12] | N-(2-fluoro-4-methyl-5- (2-(oxetan-3-ylamino)- 8,9- dihydroimidazo[1′2′:1,6] pyrido[2,3-d]pyrimidin- 6-yl)phenyl)-N-(4- fluorophenyl) cyclopropane-1,1- dicarboxamide |
| 345 | 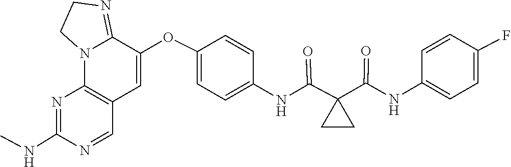 [SMILES: CNc1ncc2c(n1)N1CCN=C1C(Oc1ccc(NC(=O)C3(C(=O)Nc4ccc(F)cc4)CC3)cc1)=C2] | N-(4-fluorophenyl)-N- (4-((2-(methylamino)- 8,9- dihydroimidazo[1′2′:1,6] pyrido[2,3-d]pyrimidin- 6- yl)oxy)phenyl) cyclopropane-1,1- dicarboxamide |
| 346 | 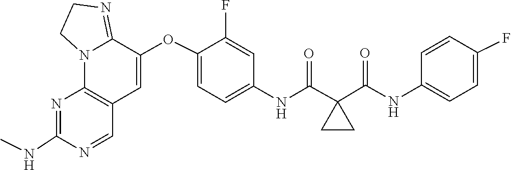 [SMILES: CNc1ncc2c(n1)N1CCN=C1C(Oc1ccc(NC(=O)C3(C(=O)Nc4ccc(F)cc4)CC3)cc1F)=C2] | N-(3-fluoro-4-((2- (methylamino)-8,9- dihydroimidazo[1′2′:1,6] pyrido[2,3-d]pyrimidin- 6-yl)oxy)phenyl)-N-(4- fluorophenyl) cyclopropane-1,1- dicarboxamide |

and pharmaceutically acceptable salts thereof.

Any formula or compound given herein, such as Formula (I), (I-1a), (I-2a), (I-2b), (I-3a), or (I-3b), or compounds of Table 1 or Table 2, is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of Table 1 or Table 2 is depicted with a particular stereochemical configuration, also provided herein is any alternative stereochemical configuration of the compound, as well as a mixture of stereoisomers of the compound in any ratio. For example, where a compound of Table 1 or Table 2 has a stereocenter that is in an “S” stereochemical configuration, also provided herein is enantiomer of the compound wherein that stereocenter is in an “R” stereochemical configuration. Likewise, when a compound of Table 1 or Table 2 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Table 1 or Table 2 has two or more stereocenters, also provided are any enantiomer or diastereomer of the compound. Furthermore, certain structures may exist as geometric isomers (i.e., cis and trans isomers), as tautomers, or as atropisomers. Additionally, any compound of Table 1 or Table 2 is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein, such as Formula (I), (I-1a), (I-2a), (I-2b), (I-3a), or (I-3b) is intended to refer to hydrates, solvates, and amorphous forms of such compounds, and mixtures thereof, even if such forms are not listed explicitly. In some embodiments, the solvent is water and the solvates are hydrates.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of G¹, G², R¹, R², R³, R⁴, R⁵, m, and n provided herein can be combined with every other variation or embodiment of G¹, G², R¹, R², R³, R⁴, R⁵, m, and n, as if each combination had been individually and specifically described.

### Compositions

Also provided are compositions, such as pharmaceutical compositions, that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, the present disclosure provides for a pharmaceutical composition comprising a compound described above admixed with at least one pharmaceutically acceptable carrier or excipient. In some embodiments, provided are compositions, such as pharmaceutical compositions that contain one or more compounds described herein, or a pharmaceutically acceptable salt thereof.

In some embodiments, provided is a pharmaceutically acceptable composition comprising a compound of Formula (I), (I-1a), (I-2a), (I-2b), (I-3a), (I-3b), or a compound of Table 1 or Table 2, or a pharmaceutically acceptable salt thereof. In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds that are substantially pure.

Also provided are packaged pharmaceutical compositions, comprising a pharmaceutical composition as described herein and instructions for using the composition to treat a patient suffering from a disease or condition described herein.

### Pharmaceutical Formulations

The present disclosure also provides a composition, e.g., a pharmaceutical composition, containing one or more of the compounds described herein, formulated together with a pharmaceutically acceptable carrier. Pharmaceutical compositions of the invention also can be administered in combination therapy, i.e., combined with other agents. For example, the combination therapy can include a compound as described herein combined with at least one other active agent.

Pharmaceutically acceptable carriers may include any and all carriers, excipients, stabilizers, solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the compound described herein, may be coated in a material to protect the compound from the action of acids and other natural conditions that may inactivate the compound.

Acceptable carriers, excipients, or stabilizers are nontoxic to recipients at standard dosages and concentrations to be administered, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ or polyethylene glycol (PEG).

The pharmaceutical compositions of the invention may include one or more pharmaceutically acceptable salts. A pharmaceutically acceptable salt retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The compounds having formula I-III as described herein are generally soluble in organic solvents such as chloroform, dichloromethane, ethyl acetate, ethanol, methanol, isopropanol, acetonitrile, glycerol, N,N-dimethylformamide, N,N-dimethylacetamide, dimethylsulfoxide, etc. In one embodiment, the present invention provides formulations prepared by mixing a compound having formula I-III with a pharmaceutically acceptable carrier. In one aspect, the formulation may be prepared using a method comprising: a) dissolving a described compound in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug Combinations

The methods of the embodiments comprise administering an effective amount of at least one exemplary compound of the present disclosure; optionally the compound may be administered in combination with one or more additional therapeutic agents. In some embodiments, the additional therapeutic agent is known to be useful for treating a proliferation disorder, such as a cancer, o a tumor in a subject. In some embodiments, the additional therapeutic agent is known to be useful for treating a neurodegenerative disorder.

The additional active ingredients may be administered in a separate pharmaceutical composition from at least one exemplary compound of the present disclosure or may be included with at least one exemplary compound of the present disclosure in a single pharmaceutical composition. The additional active ingredients may be administered simultaneously with, prior to, or after administration of at least one exemplary compound of the present disclosure.

### Dosages and Dosage Forms

For the prevention or treatment of disease, the appropriate dosage of compounds described herein will depend on the type of disease to be treated, the severity and course of the disease, whether the compound is administered for preventive or therapeutic purposes, mode of delivery, previous therapy, and the subject's clinical history. The compounds described herein are suitably administered to a subject at one time or over a series of treatments. Depending on the type and severity of the disease, a typical daily dosage might range from about 0.0001 mg/kg to 100 mg/kg or more, depending on the factors mentioned above. For repeated administrations over several days or longer, depending on the condition, the treatment is sustained until a desired suppression of disease symptoms occurs.

For example dosages can be 0.3 mg/kg body weight, 1 mg/kg body weight, 3 mg/kg body weight, 5 mg/kg body weight or 10 mg/kg body weight or within the range of 1-10 mg/kg. Treatment regimens may comprise administration once per week, once every two weeks, once every three weeks, once every four weeks, once per month, once every 3 months or once every three to 6 months. In other embodiments, sustained release formulations are administered, which would result in less frequent administration compared to non-sustained release formulations.

The amount of active ingredient which can be combined with a carrier material to produce a single dosage form will generally be that amount of the composition which produces a therapeutic effect, without being toxic to the subject. Generally, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

### Administration

A composition described herein can be administered via one or more routes of administration using one or more of a variety of methods known in the art. As will be appreciated by the skilled artisan, the route and/or mode of administration will vary depending upon the desired results. Routes of administration for the compounds and compositions described herein include oral, sublingual, buccal, intranasal, topical, rectal, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

### Methods of Treatment

The compounds and pharmaceutical compositions herein may be used for any suitable purpose. For example, the present compounds can be used in therapy and/or testing.

The compounds and pharmaceutical compositions herein may be used to treat and/or prevent a proliferation disorder, such as a cancer, or a tumor in an individual. In some embodiments, provided are methods of treating or preventing a proliferation disorder, such as a cancer, or a tumor in an individual, comprising administering to the individual in need thereof a compound of Formula (I), (I-1a), (I-2a), (I-2b), (I-3a), (I-3b), or a compound of Table 1 or Table 2, or a pharmaceutically acceptable salt thereof. In some embodiments, provided are methods of treating or preventing a proliferation disorder, such as a cancer, or a tumor in a subject in need thereof comprising administering to the subject a therapeutically effective amount of at least one chemical entity as described herein.

In some embodiments, the compounds of Formula (I), (I-1a), (I-2a), (I-2b), (I-3a), (I-3b), or compounds of Table 1 or Table 2, or a pharmaceutically acceptable salt thereof, are inhibitors of one or more kinases selected from the group consisting of MAPK, PDGFR, Src, PAKs, c-Kit, EphA2, EphB4, FGFR, Axl, and c-Met, and thus are all adapted to therapeutic use as antiproliferative or anti-metastatic agents (e.g., anticancer) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin (e.g., psoriasis) and benign hyperplasia of the prostate (e.g., BPH). In addition, it is expected that a compound of the present invention may possess activity against brain metastases originated from these disorders.

In some embodiments, compounds of Formula (I), (I-1a), (I-2a), (I-2b), (I-3a), (I-3b), or compounds of Table 1 or Table 2, or a pharmaceutically acceptable salt thereof, may also be useful in the treatment of additional disorders in which aberrant expression ligand/receptor interactions or activation or signaling events related to various kinases, are involved. Such disorders may include those of neuronal, glial, astrocytal, hypothalamic, and other glandular, macrophagal, epithelial, stromal, and blastocoelic nature in which aberrant function, expression, activation or signaling of tyrosine kinases are involved.

Also provided herein is the use of a compound of Formula (I), (I-1a), (I-2a), (I-2b), (I-3a), (I-3b), or a compound of Table 1 or Table 2, or a pharmaceutically acceptable salt thereof in the manufacture of a medicament for treatment of a proliferation disorder, such as a cancer, or a tumor in a subject.

In some embodiments, the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin (e.g., psoriasis) and benign hyperplasia of the prostate (e.g., BPH). In some embodiments, the compound of Formula (I), (I-1a), (I-2a), (I-2b), (I-3a), (I-3b), or a compound of Table 1 or Table 2, may possess activity against brain metastases originated from these disorders.

Also provided are methods for inhibiting an activity of one or more kinases, such as MAPK, PDGFR, Src, PAKs, c-Kit, EphA2, EphB4, FGFR, Axl, and c-Met, which method comprises administering to an individual in need thereof a therapeutically effective amount of at least one chemical entity as described herein. In some embodiments, provided are methods of inhibiting one or more kinases, such as MAPK, PDGFR, Src, PAKs, c-Kit, EphA2, EphB4, FGFR, Axl, and c-Met in a cell, comprising contacting the cell with at least one chemical entity as described herein, such as a compound of Formula (I), (I-1a), (I-2a), (I-2b), (I-3a), (I-3b), or a compound of Table 1 or Table 2, or a pharmaceutically acceptable salt thereof. Additionally provided herein is the use of at least one chemical entity as described herein, such as a compound of Formula (I), (I-1a), (I-2a), (I-2b), (I-3a), (I-3b), or a compound of Table 1 or Table 2, or a pharmaceutically acceptable salt thereof in the manufacture of a medicament for inhibiting an activity of one or more kinases, such as MAPK, PDGFR, Src, PAKs, c-Kit, EphA2, EphB4, FGFR, Axl, and c-Met of an individual.

Also provided are methods for treating and/or preventing a proliferation disorder, such as a cancer, or a tumor in a subject which method comprises administering to an individual in need thereof a therapeutically effective amount of at least one chemical entity as described herein such as a compound of Formula (I), (I-1a), (I-2a), (I-2b), (I-3a), (I-3b), or a compound of Table 1 or Table 2, or a pharmaceutically acceptable salt thereof. Additionally provided herein is the use of at least one chemical entity as described herein, such as a compound of Formula (I), (I-1a), (I-2a), (I-2b), (I-3a), (I-3b), or a compound of Table 1 or Table 2, or a pharmaceutically acceptable salt thereof in the manufacture of a medicament for treating and/or preventing a proliferation disorder, a cancer, or a tumor in a subject.

In one embodiment, the disease or condition to be treated or prevented is abnormal cell proliferation such as cancer. The term “cancer” refers to pre-cancerous conditions, non-malignant, low-grade, high-grade, and malignant cancer. Cancer of any tissue type is contemplated for treatment or prevention by the compounds disclosed herein. Exemplary types of cancer include carcinoma, lymphoma, blastoma, sarcoma, leukemia, and lymphoid malignancies. More specifically, in certain embodiments the cancer is squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Provided herein is a method of treating cancer in an individual in need thereof by administering to the individual a therapeutically effective amount of a compound or composition described herein. Also provided herein is the use of a compound or composition described herein in the manufacture of a medicament for treatment of cancer in an individual in need thereof. Also provided herein is the use of a compound or composition described herein for treatment of cancer in an individual in need thereof. Also provided herein is a compound or composition described herein for use in treatment of cancer in an individual in need thereof.

In another embodiment, the disease or condition to be treated or prevented is neurodegenerative disease. Exemplary types of neurodegenerative disease include, but are not limited to, Amyotrophic lateral sclerosis, Parkinson's disease, Alzheimer's disease, and Huntington's disease that occurs as a result of neurodegenerative processes.

In some embodiments, provided are methods of treating or preventing a neurodegenerative disease, such as Amyotrophic lateral sclerosis, Parkinson's disease, Alzheimer's disease, and Huntington's disease, comprising administering to the individual in need thereof a compound of Formula (I), (I-1a), (I-2a), (I-2b), (I-3a), (I-3b), or a compound of Table 1 or Table 2, or a pharmaceutically acceptable salt thereof. In some embodiments, provided are methods of treating or preventing a neurodegenerative disease, such as Amyotrophic lateral sclerosis, Parkinson's disease, Alzheimer's disease, and Huntington's disease, comprising administering to the subject a therapeutically effective amount of at least one chemical entity as described herein.

Provided herein is a method of treating a neurodegenerative disease in an individual in need thereof by administering to the individual a therapeutically effective amount of a compound or composition described herein. Also provided herein is the use of a compound or composition described herein in the manufacture of a medicament for treatment of a neurodegenerative disease in an individual in need thereof. Also provided herein is the use of a compound or composition described herein for treatment of a neurodegenerative disease in an individual in need thereof. Also provided herein is a compound or composition described herein for use in treatment of neurodegenerative disease in an individual in need thereof.

In one aspect, provided herein are kits containing a compound or composition described herein and instructions for use. In some embodiments, the kits may contain instructions for use in the treatment of cancer in an individual in need thereof. In other embodiments, the kits may contain instructions for use in the treatment of a neurodegenerative disease in an individual in need thereof. A kit may additionally contain any materials or equipment that may be used in the administration of the compound or composition, such as vials, syringes, or IV bags. A kit may also contain sterile packaging.

### General Synthetic Methods

Compounds of Formula (I) will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate. Unless otherwise specified, the variables are as defined above in reference to Formula (I).

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

General methods of preparing compounds described herein are depicted in exemplified methods below. Variable groups in the schemes provided herein are defined as for Formula (I), (I-1a), (I-2a), (I-2b), (I-3a), (I-3b), or any variation thereof. Other compounds described herein may be prepared by similar methods.

General synthetic methods which may be referred to for preparing the compounds of the present invention such as B1 are provided in Claudi F. et al, J. Org. Chem. 1974, 39, p. 3508. Certain starting materials may be prepared according to methods familiar to those skilled in the art and certain synthetic modifications may be done according to methods familiar to those skilled in the art. A standard procedure for preparing 2-alkyl-1-iminoquinazoline is provided in Bartra Sanmarti, M. et al., WO2011/076813.

In some embodiments, the compound of Formula (I) is synthesized via the procedure as shown in Scheme A.

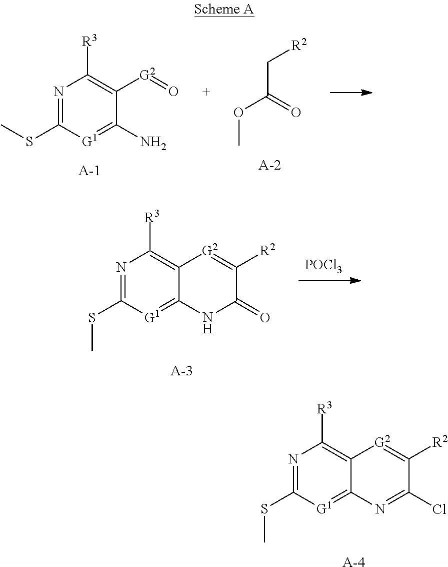

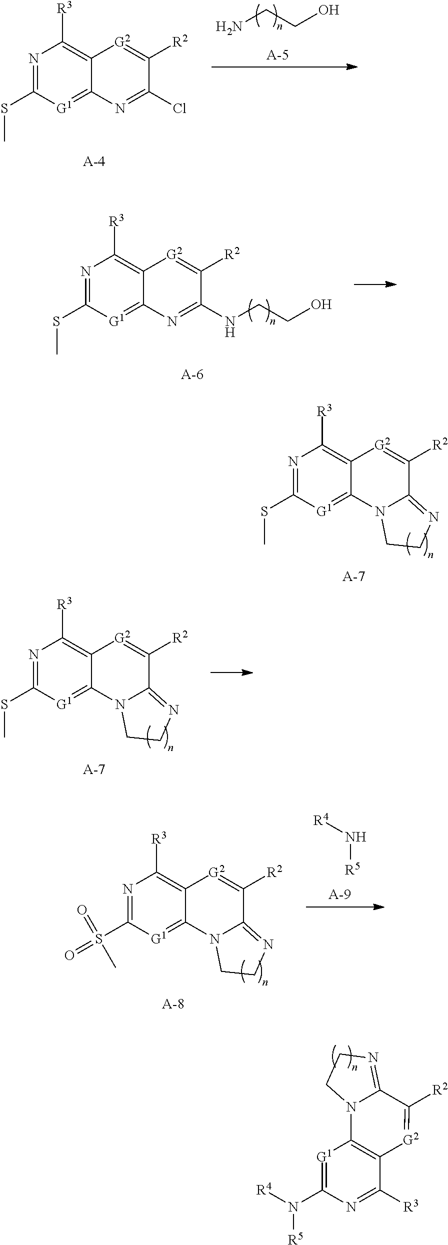

wherein G¹, G², R², R³, R⁴, R⁵, and n are as defined for Formula (I), or any variation thereof detailed herein. Particular examples are provided in the Example section below.

In some embodiments, the compound of Formula (I) is synthesized via the procedure as shown in Scheme B.

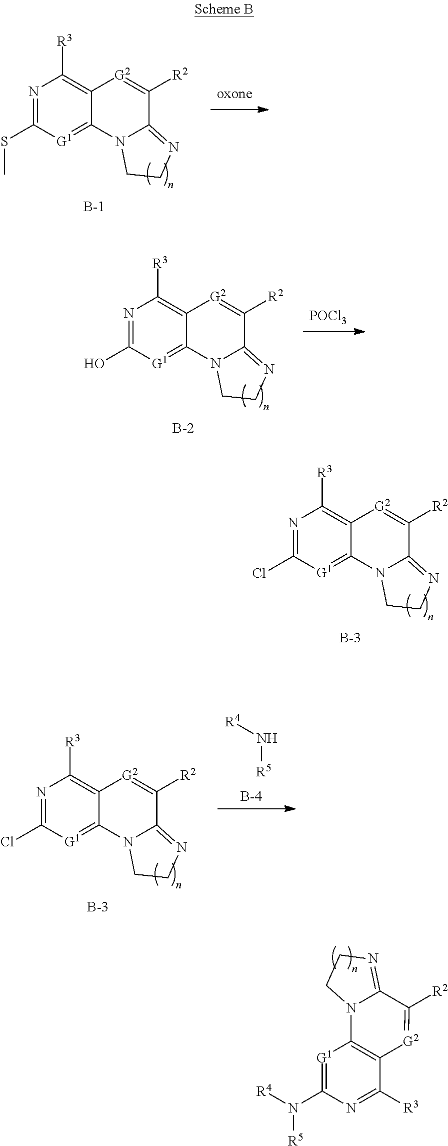

wherein G¹, G², R², R³, R⁴, R⁵, and n are as defined for Formula (I), or any variation thereof detailed herein. Particular examples are provided in the Example section below.

Starting materials, the synthesis of which is not specifically described above, are either commercially available or can be prepared using methods well known to those of skill in the art.

In some embodiments, the compound of Formula (I) is synthesized via the procedure as shown in Scheme C.

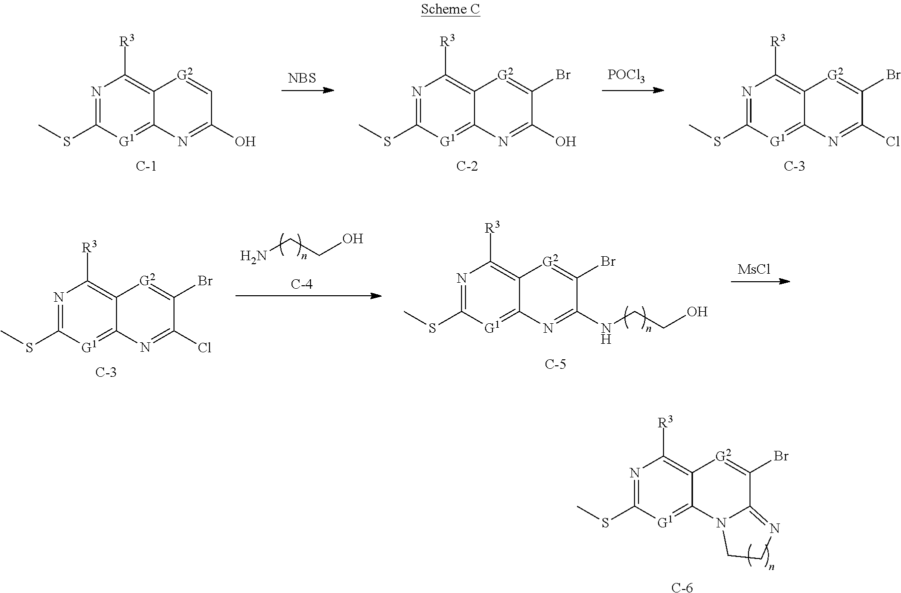

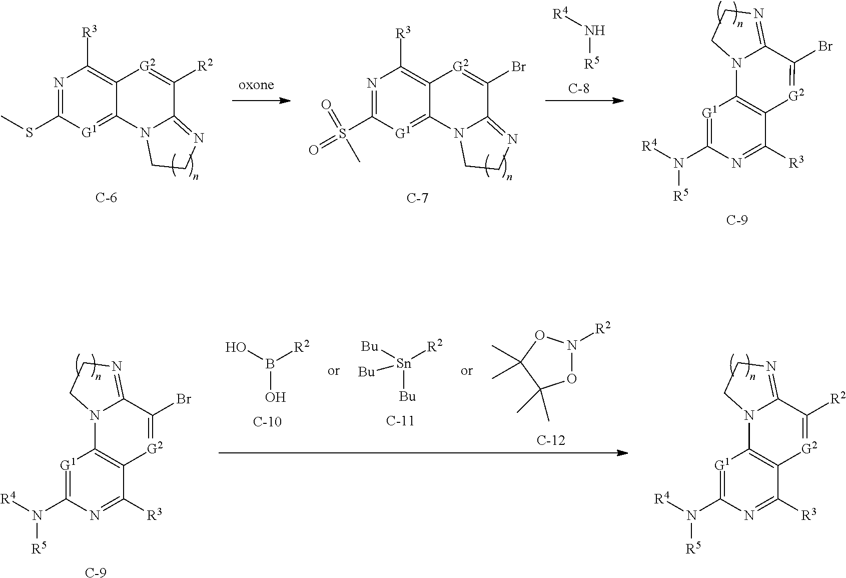

wherein G¹, G², R², R³, R⁴, R⁵, and n are as defined for Formula (I), or any variation thereof detailed herein. Particular examples are provided in the Example section below.

In some embodiments, the compound of Formula (I) is synthesized via the procedure as shown in Scheme D.

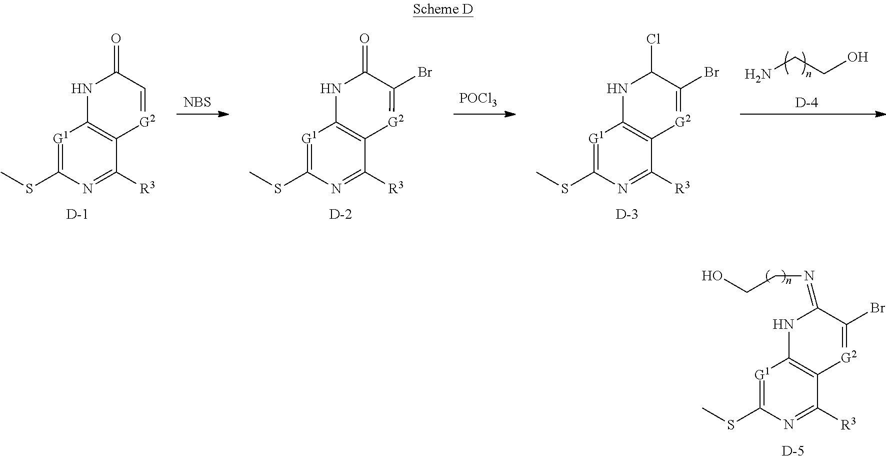

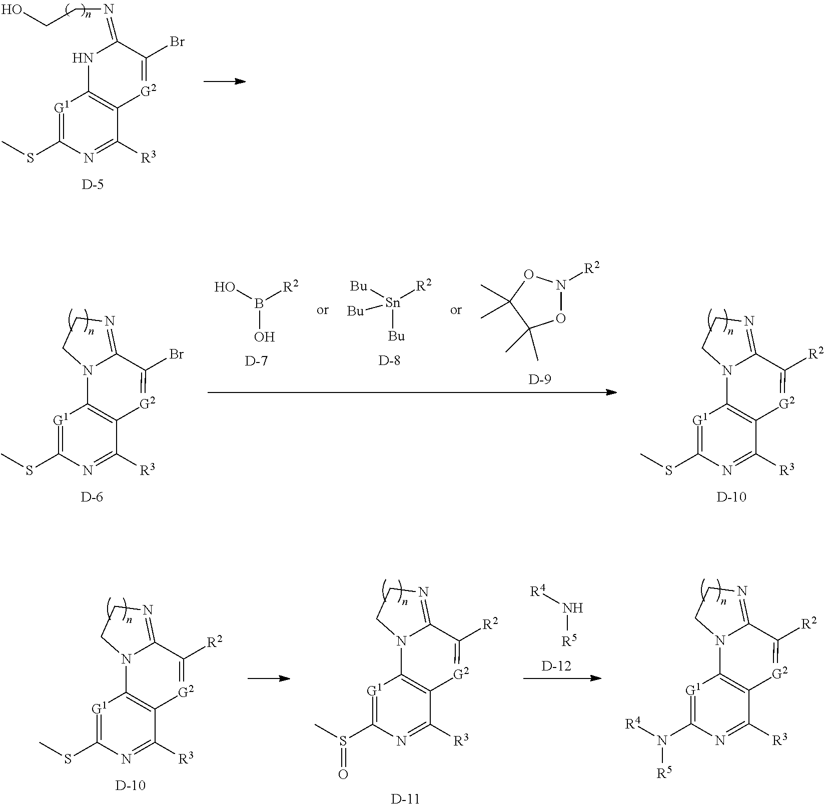

wherein G¹, G², R², R³, R⁴, R⁵, and n are as defined for Formula (I), or any variation thereof detailed herein. Particular examples are provided in the Example section below.

### EXAMPLES

The following examples are offered to illustrate but not to limit the compositions, uses, and methods provided herein. One of skill in the art will recognize that the following synthetic reactions and schemes may be modified by choice of suitable starting materials and reagents in order to access other compounds of Formula (I), (I-1a), (I-2a), (I-2b), (I-3a), or (I-3b), or a salt thereof. The compounds are prepared using the general methods described above.

The following chemical abbreviations are used throughout the Examples: ACN (acetonitrile), DCM (dichloromethane), DIEA (N, N-Diisopropyliethylamine), DMF (dimethylformamide), DMAP (4-dimethylaminopyridine), DMSO (dimethyl sulfoxide), Et₃N (triethylamine), EtOAc (ethyl acetate), ¹H NMR (proton nuclear magnetic resonance), HPLC (high-performance liquid chromatography), i-PrOH (isopropyl alcohol), KOAc (potassium acetate), LCMS (Liquid chromatography-mass spectrometry), LiHMDS (lithium bis(trimethylsilyl)amide), m-CPBA (meta-chloroperoxybenzoic acid), Mel (methyl iodide), MeOH (methanol), MsCl (methanesulfonyl chloride), NMP (N-Methyl-2-pyrrolidone), Pd₂(dba)₃ (tris(dibenzylideneacetone)dipalladium(O)), Pd(dppf)Cl₂ (1,1′-bis(diphenylphosphino)ferrocene palladium dichloride), PE (petroleum ether), SEMCl (2-(trimethylsilyl)ethoxymethylchloride), THE (tetrahydrofuran), TLC (thin layer chromatography), and TFA (trifluoroacetic acid).

### Example 1: Preparation of 6-(2,4-dichlorophenyl)-N-(3-fluoro-4-(piperazin-1-yl)phenyl)-8,9-dihydroimidazo[1′,2′:1,6]pyrido[2,3-d]pyrimidin-2-amine (Compound 1)

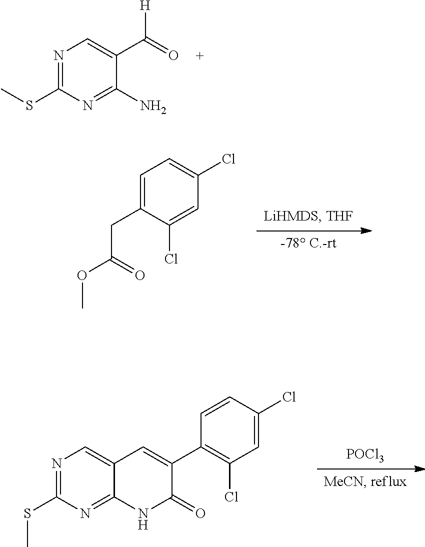

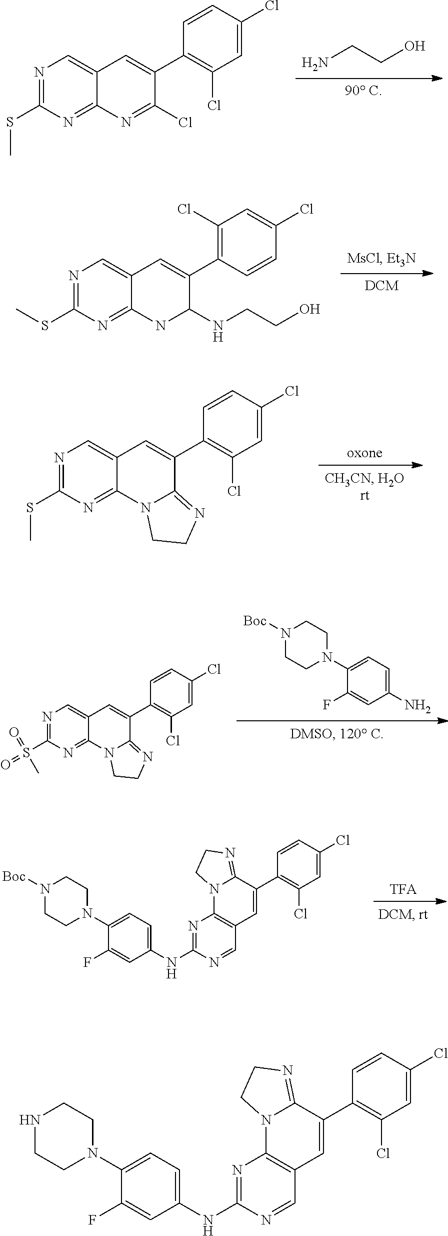

### Step 1: Synthesis of 6-(2,4-dichlorophenyl)-2-(methylthio)pyrido[2,3-d]pyrimidin-7(8H)-one

To a solution of 4-amino-2-(methylthio)pyrimidine-5-carbaldehyde (2.0 g, 11.8 mmol) in THF (100 mL) was added LiHMDS (30 mL) dropwise at −78° C. The reaction mixture was stirred at −70° C. for 2 hours. Then methyl 2-(2,4-dichlorophenyl)acetate (5.4 g, 24.8 mmol) was added, and the reaction mixture was stirred at room temperature for 16 hours. To the reaction mixture was added NH₄Cl aq (30 mL), the solid was filtered and dried in vacuum to give 6-(2,4-dichlorophenyl)-2-(methylthio)pyrido[2,3-d]pyrimidin-7(8H)-one (2.0 g, 50% yield) as a white solid. LCMS (M+H⁺) m/z: 338.0.

### Step 2: Synthesis of 7-chloro-6-(2,4-dichlorophenyl)-2-(methylthio)pyrido[2,3-d]pyrimidine

To a solution of 6-(2,4-dichlorophenyl)-2-(methylthio)pyrido[2,3-d]pyrimidin-7(8H)-one (2.0 g, 5.91 mmol) in CH₃CN (40 mL) was added POCl₃ (18.0 g, 0.12 mol). The mixture was stirred at 100° C. for 16 hours under N₂. The mixture was concentrated in vacuum to afford 7-chloro-6-(2,4-dichlorophenyl)-2-(methylthio)pyrido[2,3-d]pyrimidine (2.2 g, crude) as a white solid, which was used to the next step directly. LCMS (M+H⁺) m/z: 355.9.

### Step 3: Synthesis of 2-((6-(2,4-dichlorophenyl)-2-(methylthio)pyrido[2,3-d]pyrimidin-7-yl)amino)ethan-1-ol

A solution of 7-chloro-6-(2,4-dichlorophenyl)-2-(methylthio)pyrido[2,3-d]pyrimidine (2.2 g, crude, from Step 2) and 2-aminoethan-1-ol (20 mL) was stirred at 90° C. for 2 hours under N₂. The mixture was added to water (100 mL), filtered to afford 2-((6-(2,4-dichlorophenyl)-2-(methylthio)pyrido[2,3-d]pyrimidin-7-yl)amino)ethan-1-ol (2.1 g, crude) as a white solid, which was used to the next step directly. LCMS (M+H⁺) m/z: 381.0.

### Step 4: Synthesis of 6-(2,4-dichlorophenyl)-2-(methylthio)-8,9-dihydroimidazo[1′,2′:1,6]pyrido[2,3-d]pyrimidine

To a solution of 2-((6-(2,4-dichlorophenyl)-2-(methylthio)pyrido[2,3-d]pyrimidin-7-yl)amino)ethan-1-ol (1.0 g, 2.62 mmol) and Et₃N (794 mg, 7.86 mmol) in DCM (40 mL) was added methanesulfonyl chloride (450 mg, 3.93 mmol) at 0° C. under N₂. The reaction mixture was stirred at room temperature for 16 hours under N₂. The mixture was extracted with DCM (30 mL×3), washed with brine (50 mL), dried over Na₂SO₄, concentrated and purified by column chromatography on silica gel (DCM/MeOH=80:1) to afford 6-(2,4-dichlorophenyl)-2-(methylthio)-8,9-dihydroimidazo[1′,2′:1,6]pyrido[2,3-d]pyrimidine (615 mg, 65% yield) as a yellow solid. LCMS (M+H⁺) m/z: 363.0.

### Step 5: Synthesis of 6-(2,4-dichlorophenyl)-2-(methylsulfonyl)-8,9-dihydroimidazo[1′,2′:1,6]pyrido[2,3-d]pyrimidine

To a solution of 6-(2,4-dichlorophenyl)-2-(methylthio)-8,9-dihydroimidazo[1′,2′:1,6]pyrido[2,3-d]pyrimidine (200 mg, 0.55 mmol) in CH₃CN (5 mL) and H₂O (5 mL) was added oxone (507 mg, 0.82 mmol). The reaction mixture was stirred at 30° C. under N₂ for 48 hours. The mixture was adjusted to pH=7-8 with 1N NaOH aq, extracted with DCM (10 mL×3), bwashed with brine (50 mL), dried over Na₂SO₄, concentrated in vacuum and purified by prep-TLC (PE/EtOAc=1:1) to afford 6-(2,4-dichlorophenyl)-2-(methylsulfonyl)-8,9-dihydroimidazo[1′,2′:1,6]pyrido[2,3-d]pyrimidine (45 mg, 21% yield) as a yellow solid. LCMS (M+H⁺) m/z: 395.0.

### Step 6: Synthesis of tert-butyl 4-(4-((6-(2,4-dichlorophenyl)-8,9-dihydroimidazo[1′,2′:1,6]pyrido[2,3-d]pyrimidin-2-yl)amino)-2-fluorophenyl)piperazine-1-carboxylate

To a solution of 6-(2,4-dichlorophenyl)-2-(methylsulfonyl)-8,9-dihydroimidazo[1′,2′:1,6]pyrido[2,3-d]pyrimidine (45 mg, 0.11 mmol) in DMSO (3 mL) was added tert-butyl 4-(4-amino-2-fluorophenyl)piperazine-1-carboxylate (33 mg, 0.11 mmol). The reaction mixture was stirred at 120° C. for 1 hour under N₂. The mixture was added to water, extracted with EtOAc (10 mL×3), washed with brine (50 mL), dried over Na₂SO₄, concentrated in vacuum and purified by column chromatography on silica gel (PE/EtOAc=1:1) to afford tert-butyl 4-(4-((6-(2,4-dichlorophenyl)-8,9-dihydroimidazo[1′,2′:1,6]pyrido[2,3-d]pyrimidin-2-yl)amino)-2-fluorophenyl)piperazine-1-carboxylate (40 mg, 57% yield) as black oil. LCMS (M+H⁺) m/z: 610.2.

### Step 7: Synthesis of 6-(2,4-dichlorophenyl)-N-(3-fluoro-4-(piperazin-1-yl)phenyl)-8,9-dihydroimidazo[1′,2′:1,6]pyrido[2,3-d]pyrimidin-2-amine

To a solution of tert-butyl 4-(4-((6-(2,4-dichlorophenyl)-8,9-dihydroimidazo[1′,2′:1,6]pyrido[2,3-d]pyrimidin-2-yl)amino)-2-fluorophenyl)piperazine-1-carboxylate (40 mg, 0.065 mmol) in DCM (1 mL), was added TFA (1 mL). The mixture was stirred at rt for 2 hours under N₂. The residue was concentrated in vacuum and purified by prep-HPLC (0.1% TFA, MeCN in H₂O) to give 6-(2,4-dichlorophenyl)-N-(3-fluoro-4-(piperazin-1-yl)phenyl)-8,9-dihydroimidazo[1′,2′:1,6]pyrido[2,3-d]pyrimidin-2-amine (8.0 mg, 24% yield, TFA salt) as a white solid. ¹H NMR (400 MHz, CD₃OD): δ 8.98 (s, 1H), 8.19 (s, 1H), 7.88-7.84 (m, 1H), 7.74 (d, J=2.0 Hz, 1H), 7.57-7.48 (m, 3H), 7.14 (t, J=9.2 Hz, 1H), 4.92-4.86 (m, 2H), 4.22-4.17 (m, 2H), 3.42-3.38 (m, 4H), 3.34-3.32 (m, 4H). LCMS (M+H⁺) m/z: 510.3.

### Example 2: Preparation of 6-(2,4-dichlorophenyl)-N-(3-fluoro-4-(4-methylpiperazin-1-yl)phenyl)-8,9-dihydroimidazo[1′,2′:1,6]pyrido[2,3-d]pyrimidin-2-amine (Compound 2)

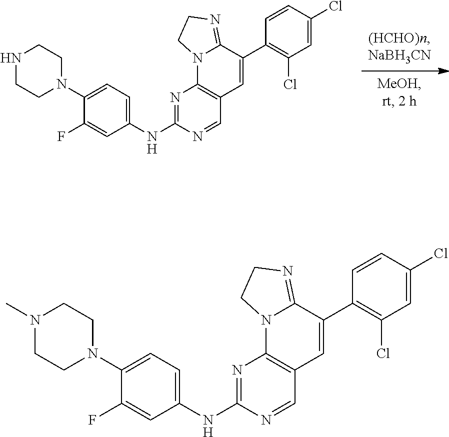 [SMILES: Fc1cc(Nc2ncc3c(n2)N2CCN=C2C(c2ccc(Cl)cc2Cl)=C3)ccc1N1CCNCC1 ; "(HCHO)n,NaBH3CN" ; "MeOH,rt, 2 h" ; CN1CCN(c2ccc(Nc3ncc4c(n3)N3CCN=C3C(c3ccc(Cl)cc3Cl)=C4)cc2F)CC1]

### Step 1: Synthesis of 6-(2,4-dichlorophenyl)-N-(3-fluoro-4-(4-methylpiperazin-1-yl)phenyl)-8,9-dihydroimidazo[1′,2′:1,6]pyrido[2,3-d]pyrimidin-2-amine

To a solution of 6-(2,4-dichlorophenyl)-N-(3-fluoro-4-(piperazin-1-yl)phenyl)-8,9-dihydroimidazo[1′,2′:1,6]pyrido[2,3-d]pyrimidin-2-amine (20 mg, 0.039 mmol) in MeOH (2 mL) was added paraformaldehyde (2 mg, 0.078 mmol). After stirring for 15 minutes, NaBH₃CN (7 mg, 0.078 mmol) was added. The mixture was stirred at room temperature for 2 hours under N₂. The residue was purified by Prep-HPLC (0.1% TFA/CH₃CN/H₂O) to give 6-(2,4-dichlorophenyl)-N-(3-fluoro-4-(4-methylpiperazin-1-yl)phenyl)-8,9-dihydroimidazo[1′,2′:1,6]pyrido[2,3-d]pyrimidin-2-amine (4.8 mg, 22% yield, TFA salt) as a yellow solid. ¹H NMR (400 MHz, DMSO-d₆): δ 9.06 (s, 1H), 8.26 (s, 1H), 7.87 (s, 1H), 7.80 (d, J=12.8 Hz, 1H), 7.65-7.54 (m, 3H), 7.16 (t, J=9.2 Hz, 1H), 4.80-4.66 (m, 2H), 4.16-4.06 (m, 2H), 3.66-3.46 (m, 4H), 3.30-3.18 (m, 2H), 3.08-2.98 (m, 2H), 2.88 (s, 3H). LCMS (M+H⁺) m/z: 524.2.

### Example 3: Preparation of 6-(2,4-dichlorophenyl)-N-(4-(4-ethylpiperazin-1-yl)-3-fluorophenyl)-8,9-dihydroimidazo[1′,2′:1,6]pyrido[2,3-d]pyrimidin-2-amine (Compound 3)

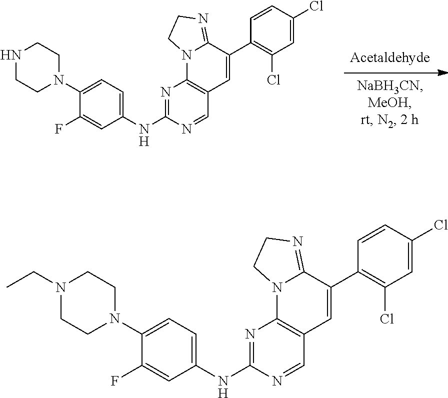 [SMILES: Fc1cc(Nc2ncc3c(n2)N2CCN=C2C(c2ccc(Cl)cc2Cl)=C3)ccc1N1CCNCC1 ; "Acetaldehyde" ; "NaBH3CN,MeOH,rt, N2, 2 h" ; CCN1CCN(c2ccc(Nc3ncc4c(n3)N3CCN=C3C(c3ccc(Cl)cc3Cl)=C4)cc2F)CC1]

### Step 1: Synthesis of 6-(2,4-dichlorophenyl)-N-(4-(4-ethylpiperazin-1-yl)-3-fluorophenyl)-8,9-dihydroimidazo[1′,2′:1,6]pyrido[2,3-d]pyrimidin-2-amine

To a solution of 6-(2,4-dichlorophenyl)-N-(3-fluoro-4-(piperazin-1-yl)phenyl)-8,9-dihydroimidazo[1′,2′:1,6]pyrido[2,3-d]pyrimidin-2-amine (22 mg, 0.043 mmol) in MeOH (2 mL) was added Acetaldehyde (0.1 mL, 0.086 mmol). After stirring for 15 minutes, NaBH₃CN (8 mg, 0.086 mmol) was added. The mixture was stirred at room temperature for 2 hours under N₂. The residue was purified by prep-HPLC (0.1% TFA/CH₃CN/H₂O) to give 6-(2,4-dichlorophenyl)-N-(4-(4-ethylpiperazin-1-yl)-3-fluorophenyl)-8,9-dihydroimidazo[1′,2′:1,6]pyrido[2,3-d]pyrimidin-2-amine (4.3 mg, 18% yield, TFA salt) as a yellow solid. ¹H NMR (400 MHz, DMSO-d₆): δ 9.02 (s, 1H), 8.23-8.20 (m, 1H), 7.88 (s, 1H), 7.81 (d, J=14.4 Hz, 1H), 7.66-7.53 (m, 3H), 7.15 (t, J=9.2 Hz, 1H), 4.76-4.60 (m, 2H), 4.09 (t, J=9.6 Hz, 2H), 3.64-3.52 (m, 2H), 3.26-3.96 (m, 8H), 1.26 (t, J=7.2 Hz, 3H). LCMS (M+H⁺) m/z: 538.2.

### Example 4: Preparation of 6-(2,4-dichlorophenyl)-N-ethyl-8,9-dihydroimidazo[1′,2′:1,6]pyrido[2,3-d]pyrimidin-2-amine (Compound 4)

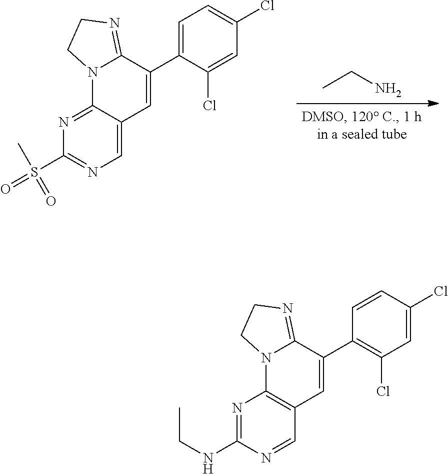

### Step 1: Synthesis of 6-(2,4-dichlorophenyl)-N-ethyl-8,9-dihydroimidazo[1′,2′:1,6]pyrido[2,3-d]pyrimidin-2-amine

To a solution of 6-(2,4-dichlorophenyl)-2-(methylsulfonyl)-8,9-dihydroimidazo[1′,2′:1,6]pyrido[2,3-d]pyrimidine (80 mg, 0.20 mmol) in DMF (4 mL) was added ethanamine (2 mL, 1M in THF). The mixture was stirred at 120° C. in a sealed tube for 1 hour. The residue was purified by prep-HPLC (0.1% TFA/CH₃CN/H₂O) to give 6-(2,4-dichlorophenyl)-N-ethyl-8,9-dihydroimidazo[1′,2′:1,6]pyrido[2,3-d]pyrimidin-2-amine (53 mg, 56% yield, TFA salt) as a yellow solid. ¹H NMR (400 MHz, DMSO-d₆): δ 8.92 (d, J=31.6 Hz, 1H), 8.18 (s, 1H), 7.86 (d, J=2.0 Hz, 1H), 7.62 (dd, J=8.4, 2.0 Hz, 1H), 7.52 (d, J=8.4 Hz, 1H), 4.66-4.60 (m, 2H), 4.06-4.03 (m, 2H), 3.51-3.42 (m, 2H), 1.24-1.14 (m, 3H). LCMS (M+H⁺) m/z: 360.1.

### Example 5: Preparation of 6-(2,4-dichlorophenyl)-N-(tetrahydro-2H-pyran-4-yl)-8,9-dihydroimidazo[1′,2′:1,6]pyrido[2,3-d]pyrimidin-2-amine (Compound 5)

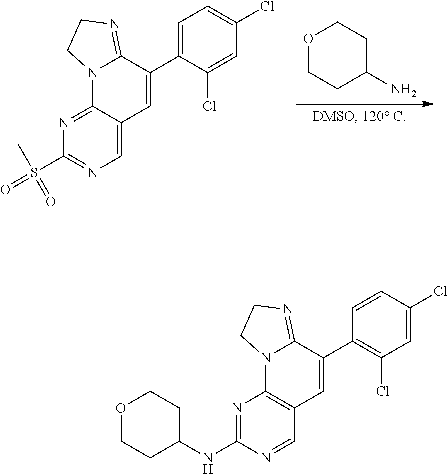

### Step 1: Synthesis of 6-(2,4-dichlorophenyl)-N-(tetrahydro-2H-pyran-4-yl)-8,9-dihydroimidazo[1′,2′:1,6]pyrido[2,3-d]pyrimidin-2-amine

To a solution of 6-(2,4-dichlorophenyl)-2-(methylsulfonyl)-8,9-dihydroimidazo[1′,2′:1,6]pyrido[2,3-d]pyrimidine (80 mg, 0.20 mmol) in DMF (4 mL) was added tetrahydro-2H-pyran-4-amine (24 mg, 0.24 mmol). The mixture was stirred at 120° C. under N₂ for 1 hour. The residue was purified by prep-HPLC (0.1% TFA/CH₃CN/H₂O) to give 6-(2,4-dichlorophenyl)-N-(tetrahydro-2H-pyran-4-yl)-8,9-dihydroimidazo[1′,2′:1,6]pyrido[2,3-d]pyrimidin-2-amine (40.48 mg, 39% yield, TFA salt) as a yellow solid. ¹H NMR (400 MHz, DMSO-d₆): δ 8.93 (d, J=23.2 Hz, 1H), 8.17 (s, 1H), 7.85 (d, J=2.4 Hz, 1H), 7.62 (dd, J=8.4, 2.4 Hz, 1H), 7.52 (d, J=8.4 Hz, 1H), 4.69-4.57 (m, 2H), 4.18-4.00 (m, 3H), 3.94-3.89 (m, 2H), 3.47-3.38 (m, 2H), 1.95-1.81 (m, 2H), 1.65-1.55 (m, 2H). LCMS (M+H⁺) m/z: 416.1.

### Example 6: Preparation 6-(2,4-dichlorophenyl)-N-(2-fluorophenyl)-8,9-dihydroimidazo[1′,2′:1,6]pyrido[2,3-d]pyrimidin-2-amine (Compound 6)

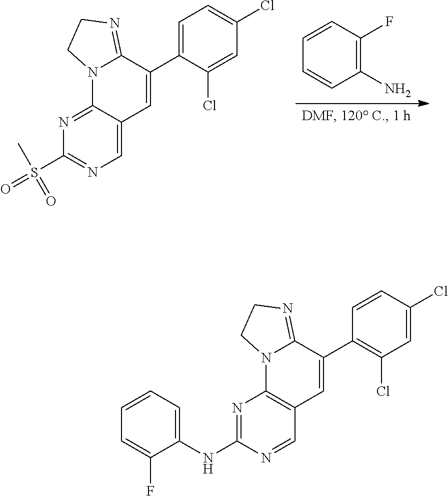

### Step 1: Synthesis of 6-(2,4-dichlorophenyl)-N-(2-fluorophenyl)-8,9-dihydroimidazo[1′,2′:1,6]pyrido[2,3-d]pyrimidin-2-amine

To a solution of 6-(2,4-dichlorophenyl)-2-(methylsulfonyl)-8,9-dihydroimidazo[1′,2′:1,6]pyrido[2,3-d]pyrimidine (90 mg, 0.22 mmol) in DMF (3 mL) was added 2-fluoroaniline (30 mg, 0.27 mmol). The mixture was stirred at 120° C. under N₂ for 1 hour. The residue was purified by prep-HPLC (0.1% TFA/CH₃CN/H₂O) to give 6-(2,4-dichlorophenyl)-N-(2-fluorophenyl)-8,9-dihydroimidazo[1′,2′:1,6]pyrido[2,3-d]pyrimidin-2-amine (17 mg, 18% yield, TFA salt) as a yellow solid. ¹H NMR (400 MHz, DMSO-d₆): δ 9.03 (s, 1H), 8.26 (s, 1H), 7.85 (d, J=2.0 Hz, 1H), 7.79-7.73 (m, 1H), 7.63 (dd, J=8.4, 2.0 Hz, 1H), 7.54 (d, J=8.4 Hz, 1H), 7.36-7.25 (m, 3H), 4.70-4.60 (m, 2H), 4.10-4.00 (m, 2H). LCMS (M+H⁺) m/z: 426.1.

### Example 7: Preparation of 6-(2,4-dichlorophenyl)-N-(2-fluoro-4-(piperazin-1-yl)phenyl)-8,9-dihydroimidazo[1′,2′:1,6]pyrido[2,3-d]pyrimidin-2-amine (Compound 7)

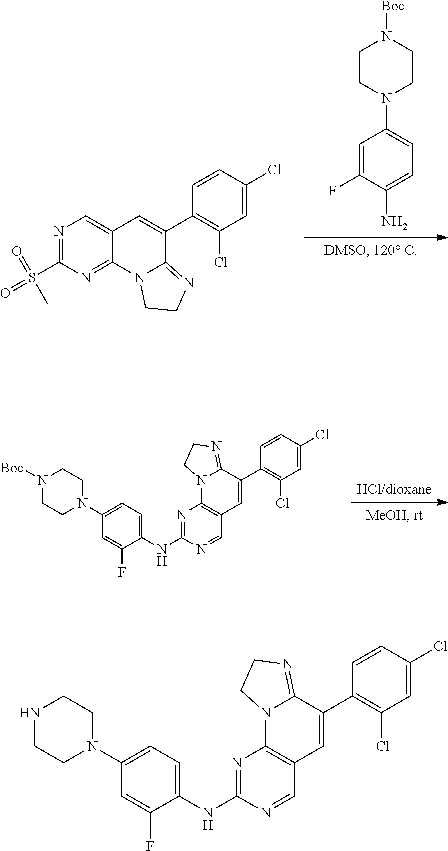

### Step 1: Synthesis of tert-butyl 4-(4-((6-(2,4-dichlorophenyl)-8,9-dihydroimidazo[1′,2′:1,6]pyrido[2,3-d]pyrimidin-2-yl)amino)-3-fluorophenyl)piperazine-1-carboxylate

To a solution of 6-(2,4-dichlorophenyl)-2-(methylsulfonyl)-8,9-dihydroimidazo[1′,2′:1,6]pyrido[2,3-d]pyrimidine (1.2 g, 3.16 mmol) in DMSO (10 mL) was added tert-butyl 4-(4-amino-3-fluorophenyl)piperazine-1-carboxylate (744 mg, 2.53 mmol). The reaction mixture was stirred at 120° C. for 2 hours under N₂. The mixture was added to water, extracted with EtOAc (10 mL×3), washed with brine (50 mL), dried over Na₂SO₄, concentrated and purified by pre-TLC (EtOAc) to afford tert-butyl 4-(4-((6-(2,4-dichlorophenyl)-8,9-dihydroimidazo[1′,2′:1,6]pyrido[2,3-d]pyrimidin-2-yl)amino)-3-fluorophenyl)piperazine-1-carboxylate (410 mg, 26% yield) as a yellow solid. LCMS (M+H⁺) m/z: 610.3.

### Step 2: Synthesis of 6-(2,4-dichlorophenyl)-N-(2-fluoro-4-(piperazin-1-yl)phenyl)-8,9-dihydroimidazo[1′,2′:1,6]pyrido[2,3-d]pyrimidin-2-amine

To a solution of tert-butyl 4-(4-((6-(2,4-dichlorophenyl)-8,9-dihydroimidazo[1′,2′:1,6]pyrido[2,3-d]pyrimidin-2-yl)amino)-3-fluorophenyl)piperazine-1-carboxylate (410 mg, 0.67 mmol) in MeOH (2 mL), was added HCl/dioxane (5 mL, 3M). The mixture was stirred at room temperature for 2 hours under N₂. The residue was concentrated to give the crude (380 mg) and 80 mg of the crude was purified by Prep-HPLC (0.1% TFA/CH₃CN/H₂O) to give 6-(2,4-dichlorophenyl)-N-(2-fluoro-4-(piperazin-1-yl)phenyl)-8,9-dihydroimidazo[1′,2′:1,6]pyrido[2,3-d]pyrimidin-2-amine (37.1 mg, 46% yield, TFA salt) as a tan solid. ¹H NMR (400 MHz, DMSO-d₆): δ 9.02-8.93 (m, 1H), 8.24 (s, 1H), 7.88 (d, J=2.0 Hz, 1H), 7.64 (d, J=8.0 Hz, 1H), 7.53 (d, J=8.4 Hz, 1H), 7.37-7.30 (m, 1H), 6.98 (d, J=14.0 Hz, 1H), 6.87 (d, J=8.8 Hz, 1H), 4.74-4.40 (m, 2H), 4.12-3.94 (m, 2H), 3.43-3.38 (m, 4H), 3.27-3.22 (m, 4H). LCMS (M+H⁺) m/z: 510.2.

### Example 8: Preparation of 6-(2,4-dichlorophenyl)-N-(2-fluoro-4-(4-methylpiperazin-1-yl)phenyl)-8,9-dihydroimidazo[1′,2′:1,6]pyrido[2,3-d]pyrimidin-2-amine (Compound 8)

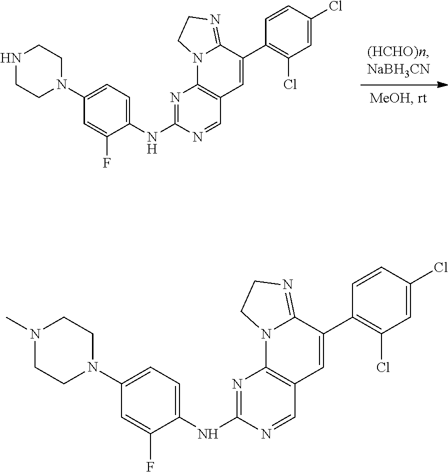 [SMILES: Fc1cc(N2CCNCC2)ccc1Nc1ncc2c(n1)N1CCN=C1C(c1ccc(Cl)cc1Cl)=C2 ; "(HCHO)n,NaBH3CN" ; "MeOH, rt" ; CN1CCN(c2ccc(Nc3ncc4c(n3)N3CCN=C3C(c3ccc(Cl)cc3Cl)=C4)c(F)c2)CC1]

### Step 1: Synthesis of 6-(2,4-dichlorophenyl)-N-(2-fluoro-4-(4-methylpiperazin-1-yl)phenyl)-8,9-dihydroimidazo[1′,2′:1,6]pyrido[2,3-d]pyrimidin-2-amine

To a solution of 6-(2,4-dichlorophenyl)-N-(2-fluoro-4-(piperazin-1-yl)phenyl)-8,9-dihydroimidazo[1′,2′:1,6]pyrido[2,3-d]pyrimidin-2-amine (65 mg, 0.12 mmol) in MeOH (3 mL) was added paraformaldehyde (7 mg, 0.25 mmol). After stirring for 15 minutes, NaBH₃CN (16 mg, 0.25 mmol) was added. The reaction mixture was stirred at room temperature for 1 hour under N₂. The residue was purified by Prep-HPLC (0.1% TFA, CH₃CN in H₂O) to give 6-(2,4-dichlorophenyl)-N-(2-fluoro-4-(4-methylpiperazin-1-yl)phenyl)-8,9-dihydroimidazo[1′,2′:1,6]pyrido[2,3-d]pyrimidin-2-amine (31.3 mg, 47% yield, TFA salt) as a yellow solid. ¹H NMR (400 MHz, DMSO-d₆): δ 9.03-8.96 (m, 1H), 8.24 (s, 1H), 7.86 (d, J=2.0 Hz, 1H), 7.65-7.62 (m, 1H), 7.53 (d, J=8.4 Hz, 1H), 7.35 (br, 1H), 6.99 (d, J=14.0 Hz, 1H), 6.88 (d, J=11.6 Hz, 1H), 4.66-4.48 (m, 2H), 4.05-4.03 (m, 2H), 3.92-3.88 (m, 2H), 3.55-3.52 (m, 2H), 3.20-3.14 (m, 2H), 3.04-2.98 (m, 2H), 2.88 (s, 3H). LCMS (M+H⁺) m/z: 524.2.

### Example 9: Preparation of 6-(2,4-dichlorophenyl)-N-(4-(4-ethylpiperazin-1-yl)-2-fluorophenyl)-8,9-dihydroimidazo[1′,2′:1,6]pyrido[2,3-d]pyrimidin-2-amine (Compound 9)

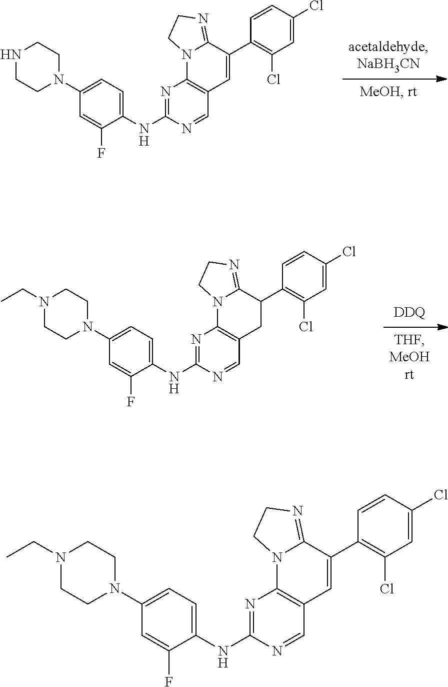

### Step 1: Synthesis of 6-(2,4-dichlorophenyl)-N-(4-(4-ethylpiperazin-1-yl)-2-fluorophenyl)-5,6,8,9-tetrahydroimidazo[1′,2′:1,6]pyrido[2,3-d]pyrimidin-2-amine

To a solution of 6-(2,4-dichlorophenyl)-N-(2-fluoro-4-(piperazin-1-yl)phenyl)-8,9-dihydroimidazo[1′,2′:1,6]pyrido[2,3-d]pyrimidin-2-amine (80 mg, 0.15 mmol) in MeOH (3 mL) was added acetaldehyde (0.1 mL, 5M in THF). After stirring for 15 minutes, NaBH₃CN (20 mg, 0.31 mmol) was added. The reaction mixture was stirred at room temperature for 2 hours under N₂. The mixture was purified by prep-HPLC (0.1% HCl, CH₃CN in H₂O) to afford 6-(2,4-dichlorophenyl)-N-(4-(4-ethylpiperazin-1-yl)-2-fluorophenyl)-5,6,8,9-tetrahydroimidazo[1′,2′:1,6]pyrido[2,3-d]pyrimidin-2-amine (25 mg, 30% yield) as a yellow solid. LCMS (M+H⁺) m/z: 540.2.

### Step 2: Synthesis of 6-(2,4-dichlorophenyl)-N-(4-(4-ethylpiperazin-1-yl)-2-fluorophenyl)-8,9-dihydroimidazo[1′,2′:1,6]pyrido[2,3-d]pyrimidin-2-amine

To a solution of 6-(2,4-dichlorophenyl)-N-(4-(4-ethylpiperazin-1-yl)-2-fluorophenyl)-5,6,8,9-tetrahydroimidazo[1′,2′:1,6]pyrido[2,3-d]pyrimidin-2-amine (22 mg, 0.040 mmol) in MeOH (1 mL) and THF (2 mL) was added DDQ (28 mg, 0.12 mmol). The mixture was stirred at room temperature for 2 hours under N₂. The residue was concentrated and purified by Prep-HPLC (0.1% TFA, CH₃CN in H₂O) to give 6-(2,4-dichlorophenyl)-N-(4-(4-ethylpiperazin-1-yl)-2-fluorophenyl)-8,9-dihydroimidazo[1′,2′:1,6]pyrido[2,3-d]pyrimidin-2-amine (5.5 mg, 25% yield, TFA salt) as a yellow solid. ¹H NMR (400 MHz, DMSO-d₆): δ 9.03-8.92 (m, 1H), 8.24 (s, 1H), 7.87 (d, J=1.6 Hz, 1H), 7.63 (dd, J=8.0, 1.6 Hz, 1H), 7.53 (d, J=8.0 Hz, 1H), 7.40 (br, 1H), 7.00 (d, J=13.2 Hz, 1H), 6.88 (d, J=9.2 Hz, 1H), 4.66-4.50 (m, 2H), 4.08-4.02 (m, 2H), 3.94-3.90 (m, 2H), 3.24-2.99 (m, 8H), 1.27 (t, J=7.2 Hz, 3H). LCMS (M+H⁺) m/z: 538.3.

### Example 10: Preparation of N-(2-fluoro-4-(piperazin-1-yl)phenyl)-8,9-dihydroimidazo[1′,2′:1,6]pyrido[2,3-d]pyrimidin-2-amine (Compound 10)

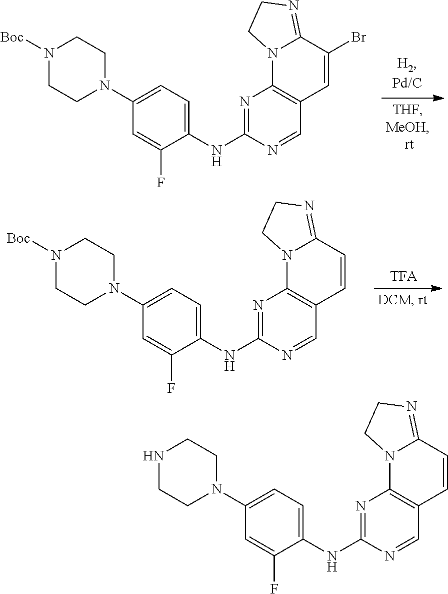 [SMILES: CC(C)(C)OC(=O)N1CCN(c2ccc(Nc3ncc4c(n3)N3CCN=C3C(Br)=C4)c(F)c2)CC1 ; CC(C)(C)OC(=O)N1CCN(c2ccc(Nc3ncc4c(n3)N3CCN=C3C=C4)c(F)c2)CC1 ; Fc1cc(N2CCNCC2)ccc1Nc1ncc2c(n1)N1CCN=C1C=C2]

### Step 1: Synthesis of tert-butyl 4-(4-((8,9-dihydroimidazo[1′,2′:1,6]pyrido[2,3-d]pyrimidin-2-yl)amino)-3-fluorophenyl)piperazine-1-carboxylate

To a solution of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (50 mg, 0.09 mmol) in MeOH (5.0 mL) and THF (5.0 mL) was added Pd/C (5 mg). The mixture was stirred at room temperature under H₂ for 16 hours. The reaction mixture was filtered, concentrated in vacuo. The residue was without purification for the next step.

### Step 2: Synthesis of N-(2-fluoro-4-(piperazin-1-yl)phenyl)-8,9-dihydroimidazo[1′,2′:1,6]pyrido[2,3-d]pyrimidin-2-amine

To a solution of tert-butyl 4-(4-((8,9-dihydroimidazo[1′,2′:1,6]pyrido[2,3-d]pyrimidin-2-yl)amino)-3-fluorophenyl)piperazine-1-carboxylate (38 mg, 0.08 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-8,9-dihydroimidazo[1′,2′:1,6]pyrido[2,3-d]pyrimidin-2-amine (15 mg, 50% yield, TFA salt) as a yellow solid. ¹H NMR (400 MHz, DMSO-d₆): δ 10.58 (br, 1H), 10.04 (br, 1H), 9.10-8.90 (m, 3H), 8.21 (d, J=9.2 Hz, 1H), 7.52-7.37 (m, 1H), 6.96 (dd, J=13.6, 2.0 Hz, 1H), 6.83 (d, J=8.4 Hz, 2H), 4.45-4.30 (m, 2H), 4.08-4.04 (m, 2H), 3.50-3.39 (m, 4H), 3.26-3.25 (m, 4H). LCMS (M+H⁺) m/z: 366.1.

### Example 11: Preparation of 6-(2-chlorophenyl)-N-(3-fluoro-4-(piperazin-1-yl)phenyl)-8,9-dihydroimidazo[1′,2′:1,6]pyrido[2,3-d]pyrimidin-2-amine (Compound 11)

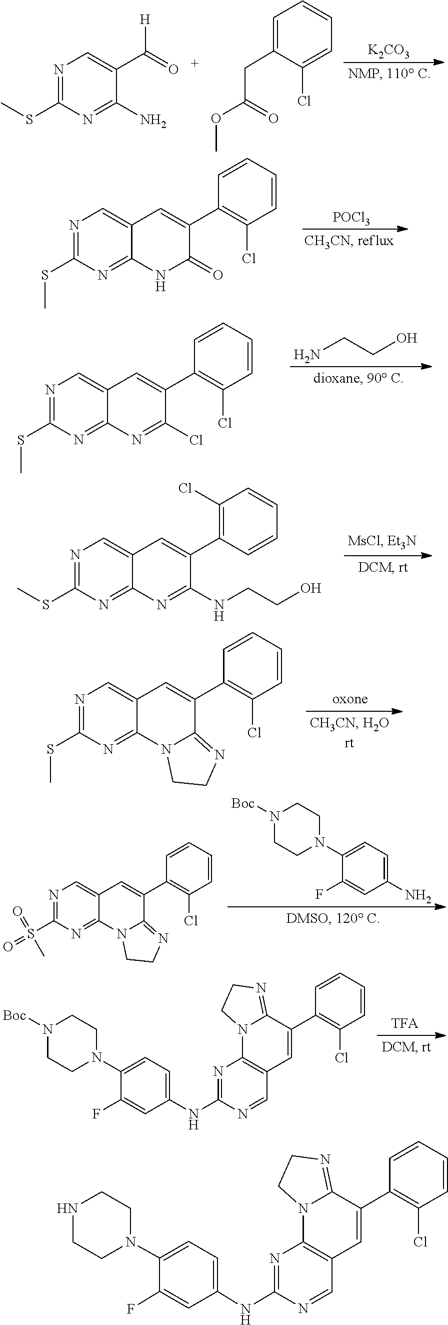

### Step 1: Synthesis of 6-(2-chlorophenyl)-2-(methylthio)pyrido[2,3-d]pyrimidin-7(8H)-one

To a solution of 4-amino-2-(methylthio)pyrimidine-5-carbaldehyde (10 g, 59.2 mmol) in NMP (130 mL) was added methyl 2-(2-chlorophenyl)acetate (22 g, 118.3 mmol) and K₂CO₃ (25 g, 181.2 mmol). The reaction mixture was stirred at 110° C. for 16 hours. The reaction mixture was cooled to room temperature and dropped into water and stirred for 1 hour. The solid was filtered and dried in vacuum to give 6-(2-chlorophenyl)-2-(methylthio)pyrido[2,3-d]pyrimidin-7(8H)-one (16.0 g, 86% yield) as a white solid.

### Step 2: Synthesis of 7-chloro-6-(2-chlorophenyl)-2-(methylthio)pyrido[2,3-d]pyrimidine

To a solution of 6-(2-chlorophenyl)-2-(methylthio)pyrido[2,3-d]pyrimidin-7(8H)-one (3.0 g, 26.4 mmol) in CH₃CN (80 mL) was POCl₃ (80 mL). The mixture was stirred at 100° C. for 16 hours. The mixture was concentrated in vacuum to afford 7-chloro-6-(2-chlorophenyl)-2-(methylthio)pyrido[2,3-d]pyrimidine (8.0 g, crude, 86% yield) as a white solid. LCMS (M+H⁺) m/z: 322.0

### Step 3: Synthesis of 2-((6-(2-chlorophenyl)-2-(methylthio)pyrido[2,3-d]pyrimidin-7-yl)amino)ethan-1-ol

A solution of 7-chloro-6-(2-chlorophenyl)-2-(methylthio)pyrido[2,3-d]pyrimidine (8.0 g, 24.8 mmol) and 2-aminoethanol (20 mL) was stirred at 90° C. for 2 hours under N₂. The mixture was poured into water (100 mL), filtered to afford 2-((6-(2-chlorophenyl)-2-(methylthio)pyrido[2,3-d]pyrimidin-7-yl)amino)ethan-1-ol (8.0 g, 93% yield) as a yellow solid. LCMS (M+H⁺) m/z: 347.1.

### Step 4: Synthesis of 6-(2-chlorophenyl)-2-(methylthio)-8,9-dihydroimidazo[1′,2′:1,6]pyrido[2,3-d]pyrimidine

To a solution of 2-((6-(2-chlorophenyl)-2-(methylthio)pyrido[2,3-d]pyrimidin-7-yl)amino)ethan-1-ol (8.0 g, 23.1 mmol) and Et₃N (11.7 g, 115.5 mmol) in DCM (200 mL) was added MsCl (8.0 g, 69.4 mmol). The reaction mixture was stirred at room temperature for 16 hours under N₂. The reaction mixture was concentrated and dropped into water and stirred for 1 hour. The solid was filtered and dried in vacuum to give 6-(2-chlorophenyl)-2-(methylthio)-8,9-dihydroimidazo[1′,2′:1,6]pyrido[2,3-d]pyrimidine (6.5 g, 86% yield) as a tan solid. LCMS (M+H⁺) m/z: 329.0.

### Step 5: Synthesis of 6-(2-chlorophenyl)-2-(methylsulfonyl)-8,9-dihydroimidazo[1′,2′:1,6]pyrido[2,3-d]pyrimidine

To a solution of 6-(2-chlorophenyl)-2-(methylthio)-8,9-dihydroimidazo[1′,2′:1,6]pyrido[2,3-d]pyrimidine (3.0 g, 9.15 mmol) in CH₃CN (50 mL) and H₂O (50 mL) was added oxone (8.40 g, 13.7 mmol). The reaction mixture was stirred at 30° C. under N₂ for 16 hours. The mixture was adjusted to pH=7-8 with 1N NaOH aq, extracted with DCM (200 mL×3), washed with brine (50 mL), dried over Na₂SO₄, concentrated in vacuum to get crude 6-(2-chlorophenyl)-2-(methylsulfonyl)-8,9-dihydroimidazo[1′,2′:1,6]pyrido[2,3-d]pyrimidine which was used to the next step without purification. CMS (M+H⁺) m/z: 361.0.

### Step 6: Synthesis of tert-butyl 4-(4-((6-(2-chlorophenyl)-8,9-dihydroimidazo[1′,2′:1,6]pyrido[2,3-d]pyrimidin-2-yl)amino)-2-fluorophenyl)piperazine-1-carboxylate

To a solution of 6-(2-chlorophenyl)-2-(methylsulfonyl)-8,9-dihydroimidazo[1′,2′:1,6]pyrido[2,3-d]pyrimidine (500 mg, 1.39 mmol) in DMSO (10 mL) was added tert-butyl 4-(4-amino-2-fluorophenyl)piperazine-1-carboxylate (200 mg, 0.68 mmol). The reaction mixture was stirred at 120° C. for 1 hour under N₂. The mixture was poured into water, extracted with EtOAc (80 mL×3), washed with brine (50 mL), dried over Na₂SO₄, concentrated in vacuum and purified by column chromatography on silica gel (DCM/CH₃OH=20:1) to afford tert-butyl 4-(4-((6-(2-chlorophenyl)-8,9-dihydroimidazo[1′,2′:1,6]pyrido[2,3-d]pyrimidin-2-yl)amino)-2-fluorophenyl)piperazine-1-carboxylate (60 mg, 8% yield) as a yellow solid. LCMS (M+H⁺) m/z: 576.2

### Step 7: Synthesis of 6-(2-chlorophenyl)-N-(3-fluoro-4-(piperazin-1-yl)phenyl)-8,9-dihydroimidazo[1′,2′:1,6]pyrido[2,3-d]pyrimidin-2-amine

To a solution of tert-butyl 4-(4-((6-(2-chlorophenyl)-8,9-dihydroimidazo[1′,2′:1,6]pyrido[2,3-d]pyrimidin-2-yl)amino)-2-fluorophenyl)piperazine-1-carboxylate (60 mg, 0.10 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give 6-(2-chlorophenyl)-N-(3-fluoro-4-(piperazin-1-yl)phenyl)-8,9-dihydroimidazo[1′,2′:1,6]pyrido[2,3-d]pyrimidin-2-amine (40 mg, 84% yield, TFA salt) as a yellow solid. ¹H NMR (400 MHz, DMSO-d₆): δ 10.85 (br, 1H), 10.24 (s, 1H), 9.07 (s, 1H), 8.93 (br, 2H), 8.30 (s, 1H), 7.80 (d, J=14.4 Hz, 1H), 7.70 (d, J=7.6 Hz, 1H), 7.62-7.51 (m, 4H), 7.15 (t, J=9.6 Hz, 1H), 4.80-4.72 (m, 2H), 4.18-4.10 (m, 2H), 3.42-3.30 (m, 4H), 3.24-3.22 (m, 4H). LCMS (M+H⁺) m/z: 476.1.

### Example 12: Preparation of 6-(2-chlorophenyl)-N-(2-fluoro-4-(piperazin-1-yl)phenyl)-8,9-dihydroimidazo[1′,2′:1,6]pyrido[2,3-d]pyrimidin-2-amine (Compound 12)

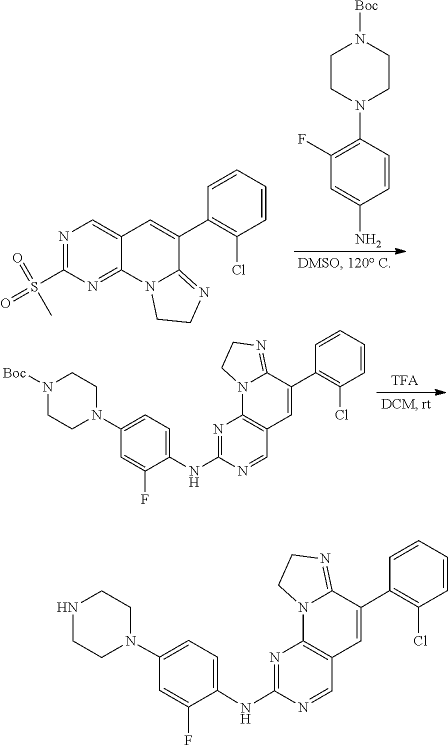 [SMILES: CS(=O)(=O)c1ncc2c(n1)N1CCN=C1C(c1ccccc1Cl)=C2 ; CC(C)(C)OC(=O)N1CCN(c2ccc(N)cc2F)CC1 ; CC(C)(C)OC(=O)N1CCN(c2ccc(Nc3ncc4c(n3)N3CCN=C3C(c3ccccc3Cl)=C4)c(F)c2)CC1 ; Fc1cc(N2CCNCC2)ccc1Nc1ncc2c(n1)N1CCN=C1C(c1ccccc1Cl)=C2]

### Step 1: Synthesis of tert-butyl 4-(4-((6-(2-chlorophenyl)-8,9-dihydroimidazo[1′,2′:1,6]pyrido[2,3-d]pyrimidin-2-yl)amino)-3-fluorophenyl)piperazine-1-carboxylate

To a solution of 6-(2-chlorophenyl)-2-(methylsulfonyl)-8,9-dihydroimidazo[1′,2′:1,6]pyrido[2,3-d]pyrimidine (500 mg, 1.39 mmol) in DMSO (10 mL) was added tert-butyl 4-(4-amino-3-fluorophenyl)piperazine-1-carboxylate (200 mg, 0.68 mmol). The reaction mixture was stirred at 120° C. for 1 hour under N₂. The mixture was poured into water, extracted with EtOAc (80 mL×3), washed with brine (50 mL), dried over Na₂SO₄, concentrated in vacuum and purified by column chromatography on silica gel (DCM/CH₃OH=20:1) to afford tert-butyl 4-(4-((6-(2-chlorophenyl)-8,9-dihydroimidazo[1′,2′:1,6]pyrido[2,3-d]pyrimidin-2-yl)amino)-3-fluorophenyl)piperazine-1-carboxylate (38 mg, 5% yield) as a yellow solid. LCMS (M+H⁺) m/z: 576.3.

### Step 2: Synthesis of 6-(2-chlorophenyl)-N-(2-fluoro-4-(piperazin-1-yl)phenyl)-8,9-dihydroimidazo[1′,2′:1,6]pyrido[2,3-d]pyrimidin-2-amine

To a solution of tert-butyl 4-(4-((6-(2-chlorophenyl)-8,9-dihydroimidazo[1′,2′:1,6]pyrido[2,3-d]pyrimidin-2-yl)amino)-3-fluorophenyl)piperazine-1-carboxylate (38 mg, 0.10 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give 6-(2-chlorophenyl)-N-(2-fluoro-4-(piperazin-1-yl)phenyl)-8,9-dihydroimidazo[1′,2′:1,6]pyrido[2,3-d]pyrimidin-2-amine (15 mg, 50% yield, TFA salt) as a yellow solid. ¹H NMR (400 MHz, DMSO-d₆): δ 10.12 (br, 2H), 8.89-8.86 (m, 3H), 8.26 (s, 1H), 7.69 (d, J=7.6 Hz, 1H), 7.61-7.50 (m, 4H), 7.98 (d, J=13.6 Hz, 1H), 6.87-6.85 (m, 1H), 4.07-4.04 (m, 2H), 3.70-3.62 (m, 2H), 3.46-3.40 (m, 4H), 3.32-3.26 (m, 4H). LCMS (M+H⁺) m/z: 476.1.

### Example 13: Preparation of (3-((6-(2-chlorophenyl)-8,9-dihydroimidazo[1′,2′:1,6]pyrido[2,3-d]pyrimidin-2-yl)amino)phenyl)methanol (Compound 13)

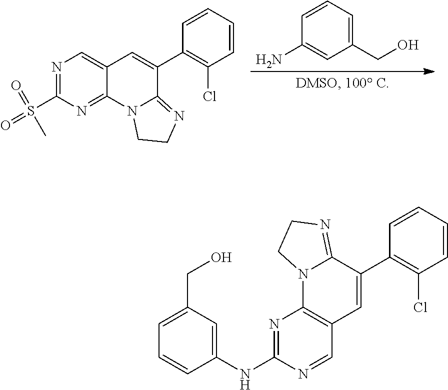 [SMILES: CS(=O)(=O)c1ncc2c(n1)N1CCN=C1C(c1ccccc1Cl)=C2 ; Nc1cccc(CO)c1 ; OCc1cccc(Nc2ncc3c(n2)N2CCN=C2C(c2ccccc2Cl)=C3)c1]

### Step 1: Synthesis of (3-((6-(2-chlorophenyl)-8,9-dihydroimidazo[1′,2′:1,6]pyrido[2,3-d]pyrimidin-2-yl)amino)phenyl)methanol

To a solution of 6-(2-chlorophenyl)-2-(methylsulfonyl)-8,9-dihydroimidazo[1′,2′:1,6]pyrido[2,3-d]pyrimidine (80 mg, 0.22 mmol) in DMSO (2 mL) was added (3-aminophenyl)methanol (41 mg, 0.33 mmol). The reaction mixture was stirred at 100° C. for 1 hour under microwave. The mixture was poured into water, extracted with EtOAc (20 mL×3), washed with brine (10 mL), dried over Na₂SO₄, concentrated in vacuum and purified by prep-HPLC (0.1% TFA, CH₃CN in H₂O) to give (3-((6-(2-chlorophenyl)-8,9-dihydroimidazo[1′,2′:1,6]pyrido[2,3-d]pyrimidin-2-yl)amino)phenyl)methanol (8.2 mg, 10% yield, TFA salt) as a yellow solid. ¹H NMR (400 MHz, CD₃OD): δ 8.96 (s, 1H), 8.16 (s, 1H), 7.81-7.79 (m, 2H), 7.65-7.63 (m, 1H), 7.56-7.51 (m, 3H), 7.40-7.38 (m, 2H), 4.63-4.60 (m, 4H), 4.18-4.16 (m, 2H). LCMS (M+H⁺) m/z: 404.1.

### Example 14: Preparation of 6-(2-chlorophenyl)-N-(1-methyl-1H-pyrazol-5-yl)-8,9-dihydroimidazo[1′,2′:1,6]pyrido[2,3-d]pyrimidin-2-amine (Compound 14)

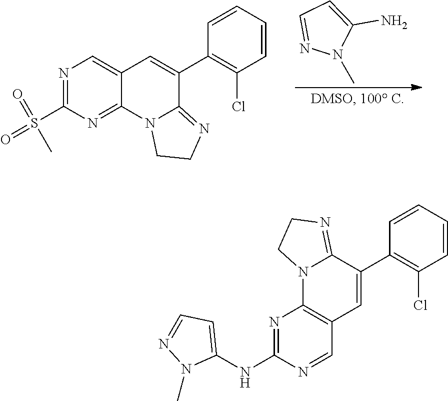

### Step 1: Synthesis of 6-(2-chlorophenyl)-N-(1-methyl-1H-pyrazol-5-yl)-8,9-dihydroimidazo[1′,2′:1,6]pyrido[2,3-d]pyrimidin-2-amine

To a solution of 6-(2-chlorophenyl)-2-(methylsulfonyl)-8,9-dihydroimidazo[1′,2′:1,6]pyrido[2,3-d]pyrimidine (100 mg, 0.28 mmol) in DMSO (5 mL) was added 1-methyl-1H-pyrazol-5-amine (35 mg, 0.36 mmol). The reaction mixture was stirred at 100° C. for 0.5 hour under microwave. The mixture was poured into water, extracted with EtOAc (20 mL×3), washed with brine (10 mL), dried over Na₂SO₄, concentrated in vacuum and purified by prep-HPLC (0.1% TFA, CH₃CN in H₂O) to give 6-(2-chlorophenyl)-N-(1-methyl-1H-pyrazol-5-yl)-8,9-dihydroimidazo[1′,2′:1,6]pyrido[2,3-d]pyrimidin-2-amine (5.2 mg, 5% yield, TFA salt) as a yellow solid. ¹H NMR (400 MHz, CD₃OD): δ 9.33 (s, 1H), 8.94 (d, J=4.0 Hz, 1H), 8.43 (s, 1H), 7.69-7.56 (m, 4H), 6.38 (d, J=4.0 Hz, 1H), 4.93 (t, J=10.0 Hz, 2H), 4.30 (t, J=10.0 Hz, 2H), 4.19 (s, 3H). LCMS (M+H⁺) m/z: 378.1.

### Example 15: Preparation of 6-(2-chlorophenyl)-N-(pyridin-3-yl)-8,9-dihydroimidazo[1′,2′:1,6]pyrido[2,3-d]pyrimidin-2-amine (Compound 15)

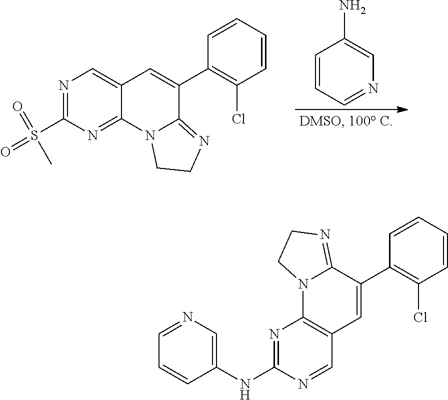 [SMILES: CS(=O)(=O)c1ncc2c(n1)N1CCN=C1C(c1ccccc1Cl)=C2 ; Nc1cccnc1 ; Clc1ccccc1C1=Cc2cnc(Nc3cccnc3)nc2N2CCN=C12]

### Step 1: Synthesis of 6-(2-chlorophenyl)-N-(pyridin-3-yl)-8,9-dihydroimidazo[1′,2′:1,6]pyrido[2,3-d]pyrimidin-2-amine

To a solution of 6-(2-chlorophenyl)-2-(methylsulfonyl)-8,9-dihydroimidazo[1′,2′:1,6]pyrido[2,3-d]pyrimidine (60 mg, 0.17 mmol) in DMSO (2 mL) was added pyridin-3-amine (19 mg, 0.20 mmol). The reaction mixture was stirred at 100° C. for 20 min under microwave. The mixture was poured into water, extracted with EtOAc (20 mL×3), washed with brine (10 mL), dried over Na₂SO₄, concentrated in vacuum and purified by prep-HPLC (0.1% TFA, CH₃CN in H₂O) to give 6-(2-chlorophenyl)-N-(pyridin-3-yl)-8,9-dihydroimidazo[1′,2′:1,6]pyrido[2,3-d]pyrimidin-2-amine (4.2 mg, 7% yield, TFA salt) as a yellow solid. ¹H NMR (400 MHz, CD₃OD): δ 9.55 (s, 1H), 9.53 (s, 1H), 9.40 (d, J=5.2 Hz, 1H), 8.54 (s, 1H), 7.98-7.97 (m, 2H), 7.71-7.57 (m, 4H), 5.05 (t, J=10.0 Hz, 2H), 4.37 (t, J=10.0 Hz, 2H). LCMS (M+H⁺) m/z: 375.1.

### Example 16: Preparation of 6-(2-chlorophenyl)-N-(pyridin-4-yl)-8,9-dihydroimidazo[1′,2′:1,6]pyrido[2,3-d]pyrimidin-2-amine (Compound 16)

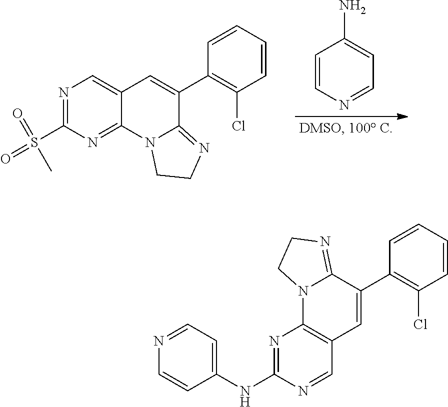 [SMILES: CS(=O)(=O)c1ncc2c(n1)N1CCN=C1C(c1ccccc1Cl)=C2 ; Nc1ccncc1 ; Clc1ccccc1C1=Cc2cnc(Nc3ccncc3)nc2N2CCN=C12]

### Step 1: Synthesis of 6-(2-chlorophenyl)-N-(pyridin-4-yl)-8,9-dihydroimidazo[1′,2′:1,6]pyrido[2,3-d]pyrimidin-2-amine

To a solution of 6-(2-chlorophenyl)-2-(methylsulfonyl)-8,9-dihydroimidazo[1′,2′:1,6]pyrido[2,3-d]pyrimidine (60 mg, 0.17 mmol) in DMSO (2 mL) was added pyridin-4-amine (19 mg, 0.20 mmol). The reaction mixture was stirred at 100° C. for 20 min under microwave. The mixture was poured into water, extracted with EtOAc (20 mL×3), washed with brine (10 mL), dried over Na₂SO₄, concentrated in vacuum and purified by prep-HPLC (0.1% TFA, CH₃CN in H₂O) to give 6-(2-chlorophenyl)-N-(pyridin-4-yl)-8,9-dihydroimidazo[1′,2′:1,6]pyrido[2,3-d]pyrimidin-2-amine (4.2 mg, 7% yield) as a yellow solid. ¹H NMR (400 MHz, CD₃OD): δ 9.47 (s, 3H), 8.51 (s, 1H), 7.70-7.57 (m, 4H), 7.12 (d, J=5.6 Hz, 2H), 5.05-5.02 (m, 2H), 4.37-4.34 (m, 2H). LCMS (M+H⁺) m/z: 375.1.

### Example 17: Preparation of 6-(2-chlorophenyl)-N-(3-methoxyphenyl)-8,9-dihydroimidazo[1′,2′:1,6]pyrido[2,3-d]pyrimidin-2-amine (Compound 17)

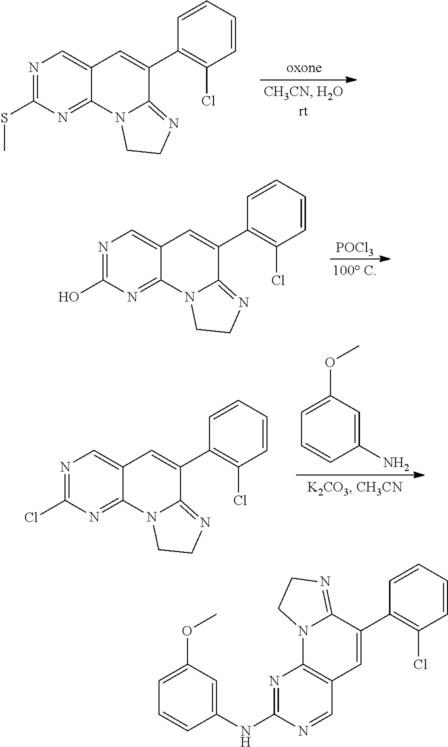

### Step 1: Synthesis of 6-(2-chlorophenyl)-8,9-dihydroimidazo[1′,2′:1,6]pyrido[2,3-d]pyrimidin-2-ol

To a solution of 6-(2-chlorophenyl)-2-(methylthio)-8,9-dihydroimidazo[1′,2′:1,6]pyrido[2,3-d]pyrimidine (2.5 g, 7.62 mmol) in CH₃CN (35 mL) and H₂O (35 mL) was added oxone (7.0 g, 11.4 mmol). The reaction mixture was stirred at 30° C. under N₂ for 16 hours. The mixture was adjusted to pH=8-9 with 1N NaOH aq, the solid was filtered to afford 6-(2-chlorophenyl)-8,9-dihydroimidazo[1′,2′:1,6]pyrido[2,3-d]pyrimidin-2-ol (2.0 g, 93% yield) as a yellow solid. LCMS (M+H⁺) m/z: 299.1.

### Step 2: Synthesis of 2-chloro-6-(2-chlorophenyl)-8,9-dihydroimidazo[1′,2′:1,6]pyrido[2,3-d]pyrimidine

A solution of 6-(2-chlorophenyl)-8,9-dihydroimidazo[1′,2′:1,6]pyrido[2,3-d]pyrimidin-2-ol (2.0 g, 6.69 mmol) in POCl₃ (30 mL) was stirred at 100° C. for 3 hours. The mixture was concentrated in vacuum to afford 2-chloro-6-(2-chlorophenyl)-8,9-dihydroimidazo[1′,2′:1,6]pyrido[2,3-d]pyrimidine (1.6 g, crude, 80% yield) as a white solid. LCMS (M+H⁺) m/z: 316.9.

### Step 3: Synthesis of 6-(2-chlorophenyl)-N-(3-methoxyphenyl)-8,9-dihydroimidazo[1′,2′:1,6]pyrido[2,3-d]pyrimidin-2-amine

A solution of 2-chloro-6-(2-chlorophenyl)-8,9-dihydroimidazo[1′,2′:1,6]pyrido[2,3-d]pyrimidine (0.2 g, 0.63 mmol) and 3-methoxyaniline (0.15 g, 1.26 mmol) and K₂CO₃ (0.17 g, 1.26 mmol) in CH₃CN (10 mL) was stirred at 150° C. for 30 min under microwave. The residue was concentrated in vacuum and purified by prep-HPLC (0.1% TFA, CH₃CN in H₂O) to give 6-(2-chlorophenyl)-N-(3-methoxyphenyl)-8,9-dihydroimidazo[1′,2′:1,6]pyrido[2,3-d]pyrimidin-2-amine (50 mg, 20% yield, TFA salt) as a yellow solid. ¹H NMR (400 MHz, DMSO-d₆): δ 10.67 (br, 1H), 10.07 (br, 1H), 9.02 (s, 1H), 8.25 (s, 1H), 7.80 (d, J=8.8 Hz, 2H), 7.70 (d, J=7.6 Hz, 1H), 7.61-7.50 (m, 3H), 6.98 (d, J=8.8 Hz, 2H), 4.73-4.70 (m, 2H), 4.08-4.07 (m, 2H), 3.77 (s, 3H). LCMS (M+H⁺) m/z: 404.1.

### Example 18: Preparation of 6-(2-chlorophenyl)-N-(3-(methylthio)phenyl)-8,9-dihydroimidazo[1′,2′:1,6]pyrido[2,3-d]pyrimidin-2-amine (Compound 18)

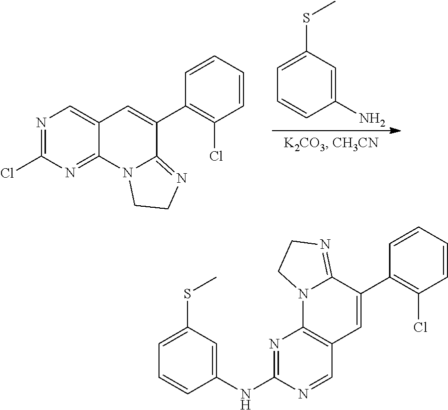

### Step 1: Synthesis of 6-(2-chlorophenyl)-N-(3-(methylthio)phenyl)-8,9-dihydroimidazo[1′,2′:1,6]pyrido[2,3-d]pyrimidin-2-amine

A solution of 2-chloro-6-(2-chlorophenyl)-8,9-dihydroimidazo[1′,2′:1,6]pyrido[2,3-d]pyrimidine (0.2 g, 0.63 mmol) and 3-(methylthio)aniline (0.18 g, 1.26 mmol) and K₂CO₃ (0.17 g, 1.26 mmol) in CH₃CN (10 mL) was stirred at 150° C. for 30 min under microwave. The residue was concentrated in vacuum and purified by prep-HPLC (0.1% TFA, CH₃CN in H₂O) to give 6-(2-chlorophenyl)-N-(3-(methylthio)phenyl)-8,9-dihydroimidazo[1′,2′:1,6]pyrido[2,3-d]pyrimidin-2-amine (50 mg, 19% yield, TFA salt) as a yellow solid. ¹H NMR (400 MHz, DMSO-d₆): δ 9.99 (br, 1H), 8.47 (s, 1H), 8.14 (s, 1H), 7.86 (s, 1H), 7.59-7.54 (m, 2H), 7.46-7.38 (m, 4H), 7.24 (t, J=8.0 Hz, 1H), 6.88 (d, J=7.2 Hz, 1H), 4.20 (t, J=9.2 Hz, 2H), 3.97 (t, J=9.2 Hz, 2H), 2.48 (s, 3H). LCMS (M+H⁺) m/z: 420.4.

### Example 19: Preparation of 6-(2-chlorophenyl)-N-(4-fluorophenyl)-8,9-dihydroimidazo[1′,2′:1,6]pyrido[2,3-d]pyrimidin-2-amine (Compound 19)

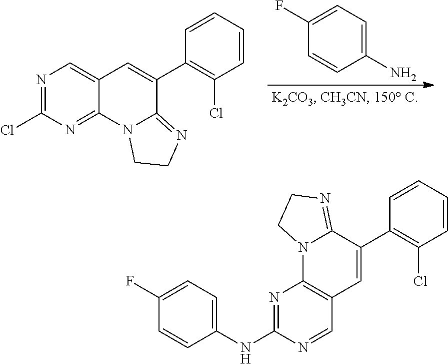

### Step 1: Synthesis of 6-(2-chlorophenyl)-N-(4-fluorophenyl)-8,9-dihydroimidazo[1′,2′:1,6]pyrido[2,3-d]pyrimidin-2-amine

A solution of 2-chloro-6-(2-chlorophenyl)-8,9-dihydroimidazo[1′,2′:1,6]pyrido[2,3-d]pyrimidine (0.2 g, 0.63 mmol) and 4-fluoroaniline (0.14 g, 1.26 mmol) and K₂CO₃ (0.17 g, 1.26 mmol) in CH₃CN (10 mL) was stirred at 150° C. for 30 min under microwave. The residue was concentrated in vacuum and purified by prep-HPLC (0.1% TFA, CH₃CN in H₂O) to give 6-(2-chlorophenyl)-N-(4-fluorophenyl)-8,9-dihydroimidazo[1′,2′:1,6]pyrido[2,3-d]pyrimidin-2-amine (30 mg, 15% yield, TFA salt) as a yellow solid. ¹H NMR (400 MHz, DMSO-d₆): δ 10.81 (br s, 1H), 10.18 (br s, 1H), 9.08 (s, 1H), 8.89 (s, 1H), 7.97-7.81 (m, 2H), 7.71-7.67 (m, 1H), 7.61-7.52 (m, 3H), 7.24 (t, J=8.8 Hz, 2H), 4.75-4.72 (m, 2H), 4.03-4.00 (m, 2H). LCMS (M+H⁺) m/z: 392.0.

### Example 20: Preparation of 6-(2-chlorophenyl)-N-(3-fluoro-4-methylphenyl)-8,9-dihydroimidazo[1′,2′:1,6]pyrido[2,3-d]pyrimidin-2-amine (Compound 20)

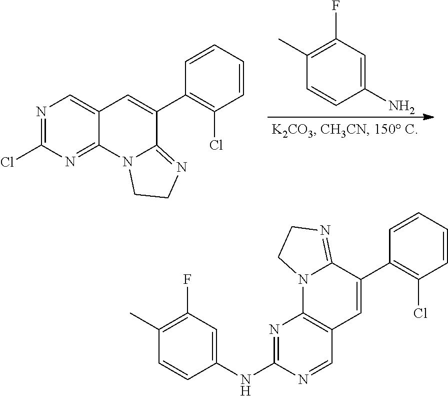

### Step 1: Synthesis of 6-(2-chlorophenyl)-N-(3-fluoro-4-methylphenyl)-8,9-dihydroimidazo[1′,2′:1,6]pyrido[2,3-d]pyrimidin-2-amine

A solution of 2-chloro-6-(2-chlorophenyl)-8,9-dihydroimidazo[1′,2′:1,6]pyrido[2,3-d]pyrimidine (0.2 g, 0.63 mmol) and 3-fluoro-4-methylaniline (0.24 g, 1.26 mmol) and K₂CO₃ (0.17 g, 1.26 mmol) in CH₃CN (10 mL) was stirred at 150° C. for 30 min under microwave. The residue was concentrated in vacuum and purified by prep-HPLC (0.1% TFA, CH₃CN in H₂O) to give 6-(2-chlorophenyl)-N-(3-fluoro-4-methylphenyl)-8,9-dihydroimidazo[1′,2′:1,6]pyrido[2,3-d]pyrimidin-2-amine (20 mg, 15% yield) as a yellow solid. ¹H NMR (400 MHz, DMSO-d₆): δ 9.99 (s, 1H), 8.40 (s, 1H), 7.80 (dd, J=8.8, 2.0 Hz, 1H), 7.53 (d, J=8.8 Hz, 1H), 7.45-7.39 (m, 4H), 7.38 (s, 1H), 7.18 (t, J=8.8 Hz, 1H), 4.16-4.11 (m, 2H), 3.98-3.94 (m, 2H), 2.18 (s, 3H). LCMS (M+H⁺) m/z: 406.1.

### Example 21: Preparation of 6-(2-chlorophenyl)-N-(oxetan-3-yl)-8,9-dihydroimidazo[1′,2′:1,6]pyrido[2,3-d]pyrimidin-2-amine (Compound 21)

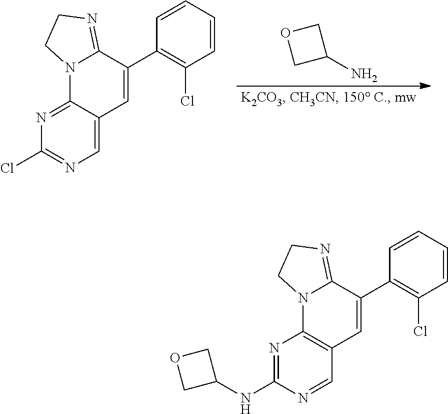 [SMILES: Clc1ncc2c(n1)N1CCN=C1C(c1ccccc1Cl)=C2 ; NC1COC1 ; Clc1ccccc1C1=Cc2cnc(NC3COC3)nc2N2CCN=C12]

### Step 1: Synthesis of 6-(2-chlorophenyl)-N-(oxetan-3-yl)-8,9-dihydroimidazo[1′,2′:1,6]pyrido[2,3-d]pyrimidin-2-amine

To a mixture of 2-chloro-6-(2-chlorophenyl)-8,9-dihydroimidazo[1′,2′:1,6]pyrido[2,3-d]pyrimidine (150 mg, 0.47 mmol), oxetan-3-amine (186 mg, 2.36 mmol) in CH₃CN (5 mL) was added K₂CO₃ (0.325 g, 2.36 mmol), and then stirred at 150° C. for 0.5 hour in microwave. The reaction mixture was concentrated in vacuum to remove solvent and then purified on prep-HPLC (0.1% NH₃HCl) to afford 6-(2-chlorophenyl)-N-(oxetan-3-yl)-8,9-dihydroimidazo[1′,2′:1,6]pyrido[2,3-d]pyrimidin-2-amine (6.4 mg, 5% yield) as a white solid. 1H NMR (400 MHz, CD₃OD): δ 8.25 (s, 1H), 7.48-7.47 (m, 1H), 7.37-7.36 (m, 3H), 7.24 (s, 1H), 5.13-5.08 (m, 2H), 4.76-4.68 (m, 3H), 4.20 (t, J=9.6 Hz, 2H), 4.00 (t, J=9.6 Hz, 2H). LCMS (M+H⁺) m/z: 354.0.

### Example 22: Preparation of 6-(2-chlorophenyl)-N-(4-(2-(dimethylamino)ethoxy)phenyl)-8,9-dihydroimidazo[1′,2′:1,6]pyrido[2,3-d]pyrimidin-2-amine (Compound 22)

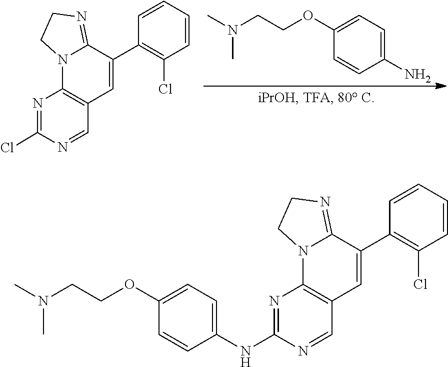

### Step 1: Synthesis of 6-(2-chlorophenyl)-N-(4-(2-(dimethylamino)ethoxy)phenyl)-8,9-dihydroimidazo[1′,2′:1,6]pyrido[2,3-d]pyrimidin-2-amine

A solution of 4-(2-(dimethylamino)ethoxy)aniline (60 mg, 0.31 mmol), TFA (0.5 mL) and 2-chloro-6-(2-chlorophenyl)-8,9-dihydroimidazo[1′,2′:1,6]pyrido[2,3-d]pyrimidine (100 mg, 0.31 mmol) in iPrOH (5 mL) was stirred at 80° C. for 6 hours. Then the mixture was diluted with water (30 mL) and then extracted with EtOAc (30 mL×3). The combined organic layers were washed with water (30 mL×2) and brine (30 mL), dried over anhydrous sodium sulphate and concentrated in vacuum. The mixture was purified on prep-HPLC (0.1% TFA, CH₃CN in H₂O) to afford 6-(2-chlorophenyl)-N-(4-(2-(dimethylamino)ethoxy)phenyl)-8,9-dihydroimidazo[1′,2′:1,6]pyrido[2,3-d]pyrimidin-2-amine (2.8 mg, 2% yield, TFA salt) a yellow solid. ¹H NMR (400 MHz, CD₃OD): δ 8.94 (s, 1H), 8.14 (s, 1H), 7.80-7.76 (m, 2H), 7.64 (d, J=7.6 Hz, 1H), 7.56-7.51 (m, 3H), 7.09-7.07 (m, 2H), 4.87-4.85 (m, 2H), 4.37-4.35 (m, 2H), 4.19-4.15 (m, 2H), 3.62-3.60 (m, 2H), 3.00 (s, 6H). LCMS (M+H⁺) m/z: 461.2.

### Example 23: Preparation 1-(3-chloro-4-(2-(methylamino)-8,9-dihydroimidazo[1′,2′:1,6]pyrido[2,3-d]pyrimidin-6-yl)phenyl)-3-methylimidazolidin-2-one (Compound 23)

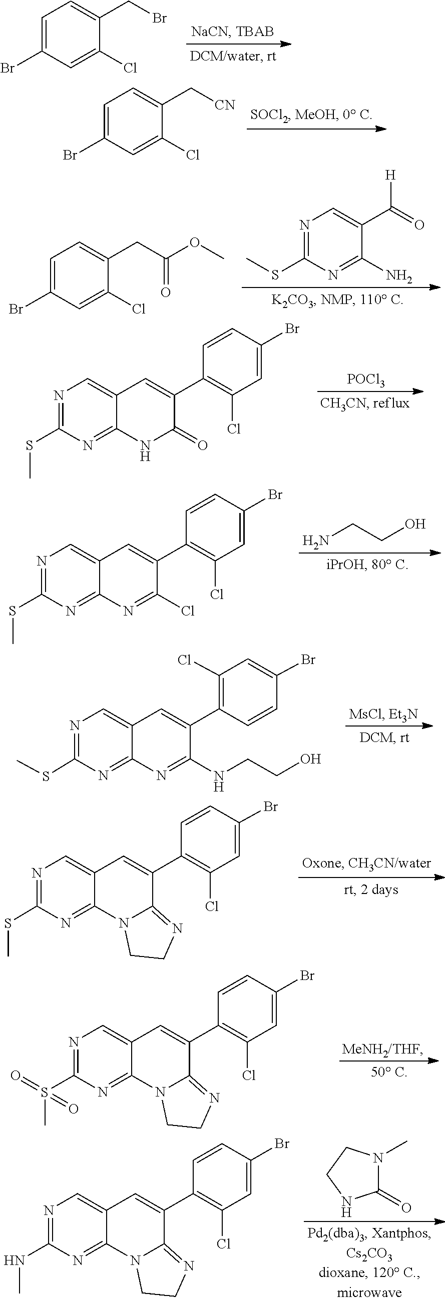

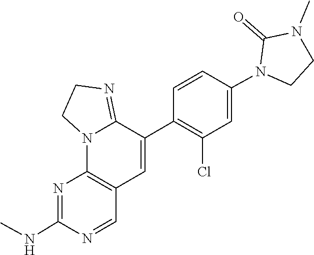 [SMILES: CNc1ncc2c(n1)N1CCN=C1C(c1ccc(N3CCN(C)C3=O)cc1Cl)=C2]

### Step 1: Synthesis of 2-(4-bromo-2-chlorophenyl)acetonitrile

To a solution of 4-bromo-1-(bromomethyl)-2-chlorobenzene (10.0 g, 35.6 mmol) and TBAB (1.05 g, 3.26 mmol) in DCM (37.5 mL) and water (37.5 mL) was added NaCN (2.43 g, 49.6 mmol). The mixture was stirred at room temperature for 18 hours. The mixture was extracted with EtOAc (50 mL×3), washed with brine (500 mL), dried over Na₂SO₄, purified by column chromatography on silica gel (PE/EtOAc=2:1) to afford 2-(4-bromo-2-chlorophenyl)acetonitrile (7.4 g, 90% yield) as a white solid.

### Step 2: Synthesis of methyl 2-(4-bromo-2-chlorophenyl)acetate

SOCl₂ (37 mL) was added dropwise into the solution of 2-(4-bromo-2-chlorophenyl)acetonitrile (7.4 g, 32 mmol) in MeOH (75 mL) at 0° C. The mixture was stirred at room temperature overnight. Then the solvent was removed and extracted with EtOAc (50 mL×3), washed with brine (500 mL), dried over Na₂SO₄, concentrated and purified by column chromatography on silica gel (PE/EtOAc=4:1) to afford methyl 2-(4-bromo-2-chlorophenyl)acetate (7.5 g, 84% yield) as colorless oil. LCMS (M+H⁺) m/z: 262.9.

### Step 3: Synthesis of 6-(4-Bromo-2-chlorophenyl)-2-(methylthio)pyrido[2,3-d]pyrimidin-7(8H)-one

A solution of methyl 2-(4-bromo-2-chlorophenyl)acetate (6.0 g, 22.9 mmol), 4-amino-2-(methylthio)pyrimidine-5-carbaldehyde (3.8 g, 22.9 mmol) and K₂CO₃ (6.3 g, 46 mmol) in NMP (60 mL) was stirred at 110° C. under N₂ for 16 hours. The mixture was poured into water and filtered to afford 6-(4-bromo-2-chlorophenyl)-2-(methylthio)pyrido[2,3-d]pyrimidin-7(8H)-one (6.6 g, 75% yield) as a brown solid. LCMS (M+H⁺) m/z: 381.9.

### Step 4: 6-(4-Bromo-2-chlorophenyl)-7-chloro-2-(methylthio)pyrido[2,3-d]pyrimidine

A solution of 6-(4-bromo-2-chlorophenyl)-2-(methylthio)pyrido[2,3-d]pyrimidin-7(8H)-one (6.6 g, 17.4 mmol) in POCl₃ (10 mL) and CH₃CN (30 mL) was stirred at 90° C. for 2 hours. The solvent was removed to afford crude 6-(4-bromo-2-chlorophenyl)-7-chloro-2-(methylthio)pyrido[2,3-d]pyrimidine (6.0 g, 86% yield) as a yellow oil. LCMS (M+H⁺) m/z: 400.2.

### Step 5: Synthesis of 2-((6-(4-bromo-2-chlorophenyl)-2-(methylthio)pyrido[2,3-d]pyrimidin-7-yl)amino)ethan-1-ol

To a solution of 6-(4-bromo-2-chlorophenyl)-7-chloro-2-(methylthio)pyrido[2,3-d]pyrimidine (6.0 g, 15 mmol) in iPrOH (5 mL) was added 2-aminoethanol (5 mL). The reaction mixture was stirred at 80° C. under N₂ for 16 hours. The mixture was filtered to afford 2-((6-(4-bromo-2-chlorophenyl)-2-(methylthio)pyrido[2,3-d]pyrimidin-7-yl)amino)ethan-1-ol (3.8 g, 60% yield) as a yellow solid. LCMS (M+H⁺) m/z: 425.0.

### Step 6: Synthesis of 6-(4-bromo-2-chlorophenyl)-2-(methylthio)-8,9-dihydroimidazo[1′,2′:1,6]pyrido[2,3-d]pyrimidine

To a solution of 2-((6-(4-bromo-2-chlorophenyl)-2-(methylthio)pyrido[2,3-d]pyrimidin-7-yl)amino)ethan-1-ol (3.8 g, 8.9 mmol) and Et₃N (1.8 g, 18 mmol) in DCM (5 mL) was added MsCl (2 g, 18 mmol) at 0° C. The reaction mixture was stirred at room temperature for 18 hours. The solvent was removed and purified by column chromatography on silica gel (DCM/MeOH=20:1) to afford 6-(4-bromo-2-chlorophenyl)-2-(methylthio)-8,9-dihydroimidazo[1′,2′:1,6]pyrido[2,3-d]pyrimidine (2.5 g, 69% yield) as a yellow solid. LCMS (M−H⁺) m/z: 407.1.

### Step 7: Synthesis of 6-(4-bromo-2-chlorophenyl)-2-(methylsulfonyl)-8,9-dihydroimidazo[1′,2′:1,6]pyrido[2,3-d]pyrimidine

To a solution of 6-(4-bromo-2-chlorophenyl)-2-(methylthio)-8,9-dihydroimidazo[1′,2′:1,6]pyrido[2,3-d]pyrimidine (1.3 g, 3.2 mmol) in CH₃CN (15 mL) and water (15 mL) was added Oxone (4.0 g, 6.4 mmol). The reaction mixture was stirred at room temperature under N₂ for 48 hours. The mixture was extracted with EtOAc (30 mL×3), washed with NH₄Cl aq (50 mL) and brine (50 mL), dried over Na₂SO₄, concentrated in vacuum to afford 6-(4-bromo-2-chlorophenyl)-2-(methylsulfonyl)-8,9-dihydroimidazo[1′,2′:1,6]pyrido[2,3-d]pyrimidine (800 mg, 57% yield) as a yellow solid. LCMS (M+H⁺) m/z: 439.0.

### Step 8: Synthesis of 6-(4-bromo-2-chlorophenyl)-N-methyl-8,9-dihydroimidazo[1′,2′:1,6]pyrido[2,3-d]pyrimidin-2-amine

To a solution of 6-(4-bromo-2-chlorophenyl)-2-(methylsulfonyl)-8,9-dihydroimidazo[1′,2′:1,6]pyrido[2,3-d]pyrimidine (800 mg, 1.8 mmol) in THF (8 mL) was added CH₃NH₂ in THF (5 mL). The mixture was stirred at 50° C. under N₂ for 2 hours. The mixture was concentrated in vacuum to afford 6-(4-bromo-2-chlorophenyl)-N-methyl-8,9-dihydroimidazo[1′,2′:1,6]pyrido[2,3-d]pyrimidin-2-amine (650 mg, 92% yield) as a yellow solid. LCMS (M+H⁺) m/z: 390.0.

### Step 9: Synthesis of 1-(3-chloro-4-(2-(methylamino)-8,9-dihydroimidazo[1′,2′:1,6]pyrido[2,3-d]pyrimidin-6-yl)phenyl)-3-methylimidazolidin-2-one

A solution of 6-(4-bromo-2-chlorophenyl)-N-methyl-8,9-dihydroimidazo[1′,2′:1,6]pyrido[2,3-d]pyrimidin-2-amine (20 mg, 0.05 mmol), 1-methylimidazolidin-2-one (10 mg, 0.1 mmol), Pd₂(dba)₃ (4.5 mg, 0.005 mmol), Xantphos (5.8 mg, 0.01 mmol), and Cs₂CO₃ (32.5 mg, 0.1 mmol) in dioxane (2 mL) was stirred at 120° C. under N₂ for 2 hours under microwave irradiation. The mixture was concentrated in vacuum and purified by prep-HPLC (0.1% TFA, CH₃CN in H₂O) to afford 1-(3-chloro-4-(2-(methylamino)-8,9-dihydroimidazo[1′,2′:1,6]pyrido[2,3-d]pyrimidin-6-yl)phenyl)-3-methylimidazolidin-2-one (5 mg, 25% yield, TFA salt) as a yellow solid. ¹H NMR (400 MHz, CD₃OD): δ 8.88-8.77 (m, 1H), 8.05 (s, 1H), 7.97 (d, J=2.0 Hz, 1H), 7.57 (dd, J=8.4, 2.0 Hz, 1H), 7.41 (d, J=8.4 Hz, 1H), 4.76-4.70 (m, 2H), 4.15 (t, J=9.2 Hz, 2H), 3.90 (t, J=8.0 Hz, 2H), 3.57 (t, J=8.0 Hz, 2H), 3.07 (s, 3H), 2.89 (s, 3H). LCMS (M+H⁺) m/z: 410.2.

### Example 24: Preparation 1-(3-chloro-4-(2-(ethylamino)-8,9-dihydroimidazo[1′,2′:1,6]pyrido[2,3-d]pyrimidin-6-yl)phenyl)-3-methylpyrazin-2(1H)-one (Compound 24)

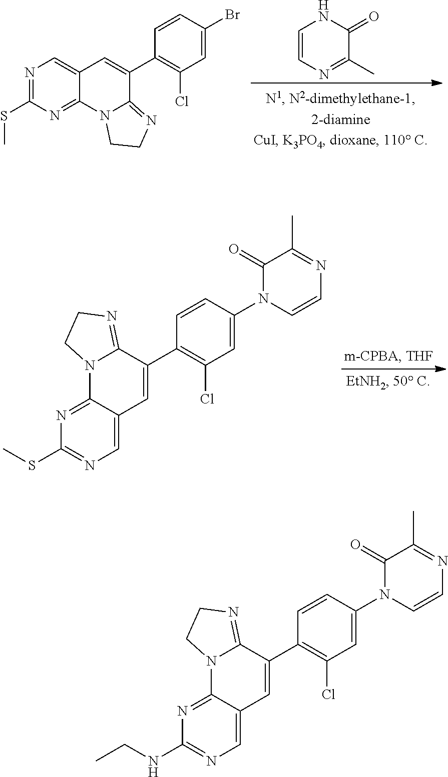

### Step 1: Synthesis of 1-(3-chloro-4-(2-(methylthio)-8,9-dihydroimidazo[1′,2′:1,6]pyrido[2,3-d]pyrimidin-6-yl)phenyl)-3-methylpyrazin-2(1H)-one

A solution of 6-(4-bromo-2-chlorophenyl)-2-(methylthio)-8,9-dihydroimidazo[1′,2′:1,6]pyrido[2,3-d]pyrimidine (100 mg, 0.24 mmol), 3-methylpyrazin-2(1H)-one (50 mg, 0.48 mmol), CuI (10 mg, 0.048 mmol), K₃PO₄ (194 mg, 0.72 mmol) and N¹,N²-dimethylethane-1,2-diamine (8.4 mg, 0.096 mmol) in dioxane (5 mL) was stirred at 110° C. under N₂ for 18 hours. The mixture was concentrated in vacuum and purified by column chromatography on silica gel (DCM/MeOH=10:1) to afford 1-(3-chloro-4-(2-(methylthio)-8,9-dihydroimidazo[1′,2′:1,6]pyrido[2,3-d]pyrimidin-6-yl)phenyl)-3-methylpyrazin-2(1H)-one (10 mg, 10% yield) as a yellow solid. LCMS (M+H⁺) m/z: 437.1.

### Step 2: Synthesis of 1-(3-chloro-4-(2-(ethylamino)-8,9-dihydroimidazo[1′,2′:1,6]pyrido[2,3-d]pyrimidin-6-yl)phenyl)-3-methylpyrazin-2(1H)-one

To a solution of 1-(3-chloro-4-(2-(methylthio)-8,9-dihydroimidazo[1′,2′:1,6]pyrido[2,3-d]pyrimidin-6-yl)phenyl)-3-methylpyrazin-2(1H)-one (10 mg, 0.022 mmol) in THF (10 mL) was added m-CPBA (10 mg, 0.057 mmol). The mixture was stirred at room temperature under N₂ for 2 hours. Then EtNH₂ in THF (1 mL) was added and the mixture was stirred at 50° C. for 2 hours. The mixture was concentrated in vacuum and purified by prep-HPLC (0.1% TFA/CH₃CN/H₂O) to afford 1-(3-chloro-4-(2-(ethylamino)-8,9-dihydroimidazo[1′,2′:1,6]pyrido[2,3-d]pyrimidin-6-yl)phenyl)-3-methylpyrazin-2(1H)-one (3.0 mg, 30% yield, TFA salt) as a yellow solid. ¹H NMR (400 MHz, CD₃OD): δ 8.90-8.82 (m, 1H), 8.14 (s, 1H), 7.85 (s, 1H), 7.68 (d, J=8.0 Hz, 1H), 7.62 (d, J=8.0 Hz, 1H), 7.46 (d, J=4.4 Hz, 1H), 7.34 (d, J=4.4 Hz, 1H), 4.79-4.77 (m, 2H), 4.18-4.15 (m, 2H), 3.60-3.55 (m, 2H), 2.46 (s, 3H), 1.32-1.24 (m, 3H). LCMS (M+H⁺) m/z: 434.1.

### Example 25: Preparation of 6-(2-chloro-4-(6-methylpyrazin-2-yl)phenyl)-N-methyl-8,9-dihydroimidazo[1′,2′:1,6]pyrido[2,3-d]pyrimidin-2-amine (Compound 25)

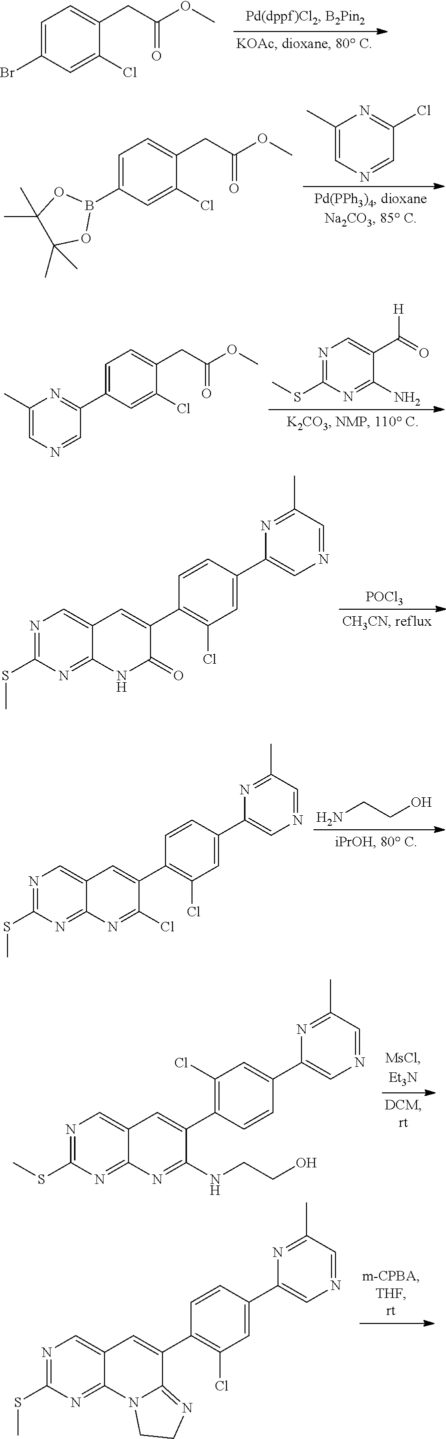

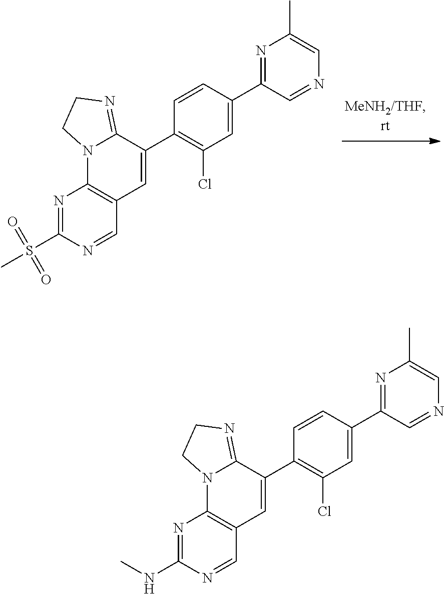 [SMILES: Cc1cncc(-c2ccc(C3=Cc4cnc(S(C)(=O)=O)nc4N4CCN=C34)c(Cl)c2)n1 ; CNc1ncc2c(n1)N1CCN=C1C(c1ccc(-c3cncc(C)n3)cc1Cl)=C2]

### Step 1: Synthesis of methyl 2-(2-chloro-4-(4,4,5,5-tetramethyl-1,3,2-dioxaborolan-2-yl)phenyl)acetate

A solution of methyl 2-(4-bromo-2-chlorophenyl)acetate (2.0 g, 7.6 mmol), 4,4,4′,4′,5,5,5′,5′-octamethyl-2,2′-bi(1,3,2-dioxaborolane) (2.13 g, 8.38 mmol), Pd(dppf)Cl₂ (500 mg, 0.68 mmol) and KOAc (2.2 g, 22.4 mmol) in dioxane (20 mL) was stirred at 80° C. for 4 hours. The mixture was extracted with EtOAc (50 mL×3), washed with brine (500 mL), dried over Na₂SO₄, purified by column chromatography on silica gel (PE/EtOAc=20:1) to afford methyl 2-(2-chloro-4-(4,4,5,5-tetramethyl-1,3,2-dioxaborolan-2-yl)phenyl)acetate (2.0 g, 84% yield) as a white solid. LCMS (M+H⁺) m/z: 311.4.

### Step 2: Synthesis of methyl 2-(2-chloro-4-(6-methylpyrazin-2-yl)phenyl)acetate

A solution of methyl 2-(2-chloro-4-(4,4,5,5-tetramethyl-1,3,2-dioxaborolan-2-yl)phenyl)acetate (1.7 g, 5.5 mmol), 2-chloro-6-methylpyrazine (650 mg, 5.0 mmol), Pd(PPh₃)₄ (557 mg, 0.5 mmol) and Na₂CO₃ (1.06 g, 10 mmol) in dioxane (20 mL) and water (2 mL) was stirred at 85° C. for 18 hours. The mixture was extracted with EtOAc (50 mL×3), washed with brine (500 mL), dried over Na₂SO₄, purified by column chromatography on silica gel (PE/EtOAc=20:1) to afford methyl 2-(2-chloro-4-(6-methylpyrazin-2-yl)phenyl)acetate (600 mg, 40% yield) as a white solid. LCMS (M+H⁺) m/z: 277.1.

### Step 3: Synthesis of 6-(2-chloro-4-(6-methylpyrazin-2-yl)phenyl)-2-(methylthio)pyrido[2,3-d]pyrimidin-7(8H)-one

A solution of methyl 2-(2-chloro-4-(6-methylpyrazin-2-yl)phenyl)acetate (600 mg, 2.17 mmol), 4-amino-2-(methylthio)pyrimidine-5-carbaldehyde (366 mg, 2.17 mmol) and K₂CO₃ (603 mg, 4.34 mmol) in NMP (10 mL) was stirred at 110° C. under N₂ for 16 hours. The mixture was poured into water and filtered to afford 6-(2-chloro-4-(6-methylpyrazin-2-yl)phenyl)-2-(methylthio)pyrido[2,3-d]pyrimidin-7(8H)-one (300 mg, 35% yield) as a brown solid. LCMS (M+H⁺) m/z: 396.1.

### Step 4: 7-Chloro-6-(2-chloro-4-(6-methylpyrazin-2-yl)phenyl)-2-(methylthio)pyrido[2,3-d]pyrimidine

A solution of 6-(2-chloro-4-(6-methylpyrazin-2-yl)phenyl)-2-(methylthio)pyrido[2,3-d]pyrimidin-7(8H)-one (300 mg, 0.76 mmol) in POCl₃ (2 mL) and CH₃CN (6 mL) was stirred at 90° C. for 2 hours. The solvent was removed to afford crude 7-chloro-6-(2-chloro-4-(6-methylpyrazin-2-yl)phenyl)-2-(methylthio)pyrido[2,3-d]pyrimidine (270 mg, 86% yield) as a yellow oil. LCMS (M+H⁺) m/z: 414.1.

### Step 5: Synthesis of 2-((6-(2-chloro-4-(6-methylpyrazin-2-yl)phenyl)-2-(methylthio)pyrido[2,3-d]pyrimidin-7-yl)amino)ethan-1-ol

To a solution of 7-chloro-6-(2-chloro-4-(6-methylpyrazin-2-yl)phenyl)-2-(methylthio)pyrido[2,3-d]pyrimidine (250 mg, 0.60 mmol) in i-PrOH (5 mL) was added 2-aminoethanol (0.5 mL). The reaction mixture was stirred at 80° C. under N₂ for 16 hours. The mixture was filtered to afford 2-((6-(2-chloro-4-(6-methylpyrazin-2-yl)phenyl)-2-(methylthio)pyrido[2,3-d]pyrimidin-7-yl)amino)ethan-1-ol (200 mg, 76% yield) as a yellow solid. LCMS (M+H⁺) m/z: 439.2.

### Step 6: Synthesis of 6-(2-chloro-4-(6-methylpyrazin-2-yl)phenyl)-2-(methylthio)-8,9-dihydroimidazo[1′,2′:1,6]pyrido[2,3-d]pyrimidine

To a solution of 2-((6-(2-chloro-4-(6-methylpyrazin-2-yl)phenyl)-2-(methylthio)pyrido[2,3-d]pyrimidin-7-yl)amino)ethan-1-ol (50 mg, 0.114 mmol) and Et₃N (35 mg, 0.342 mmol) in DCM (5 mL) was added MsCl (26 mg, 0.228 mmol) at 0° C. The reaction mixture was stirred at room temperature for 18 hours. The solvent was removed and purified by column chromatography on silica gel (DCM/MeOH=20:1) to afford 6-(2-chloro-4-(6-methylpyrazin-2-yl)phenyl)-2-(methylthio)-8,9-dihydroimidazo[1′,2′:1,6]pyrido[2,3-d]pyrimidine (40 mg, 83% yield) as a yellow solid. LCMS (M+H⁺) m/z: 421.1.

### Step 7: Synthesis of 6-(2-chloro-4-(6-methylpyrazin-2-yl)phenyl)-2-(methylsulfonyl)-8,9-dihydroimidazo[1′,2′:1,6]pyrido[2,3-d]pyrimidine

To a solution of 6-(2-chloro-4-(6-methylpyrazin-2-yl)phenyl)-2-(methylthio)-8,9-dihydroimidazo[1′,2′:1,6]pyrido[2,3-d]pyrimidine (20 mg, 0.047 mmol) in THF (2 mL) was added m-CPBA (19 mg, 0.095 mmol). The reaction mixture was stirred at room temperature under N₂ for 20 min. The solvent was removed to afford crude 6-(2-chloro-4-(6-methylpyrazin-2-yl)phenyl)-2-(methylsulfonyl)-8,9-dihydroimidazo[1′,2′:1,6]pyrido[2,3-d]pyrimidine (20 mg, 90% yield) as a yellow solid. LCMS (M+H⁺) m/z: 453.1.

### Step 8: Synthesis of 6-(2-chloro-4-(6-methylpyrazin-2-yl)phenyl)-N-methyl-8,9-dihydroimidazo[1′,2′:1,6]pyrido[2,3-d]pyrimidin-2-amine

To a solution of 6-(2-chloro-4-(6-methylpyrazin-2-yl)phenyl)-2-(methylsulfonyl)-8,9-dihydroimidazo[1′,2′:1,6]pyrido[2,3-d]pyrimidine (20 mg, 0.044 mmol) in THF (8 mL) was added CH₃NH₂ in THF (5 mL). The mixture was stirred at room temperature under N₂ for 0.5 hour. The mixture was concentrated in vacuum and purified by prep-HPLC (0.1% TFA, CH₃CN in H₂O) to afford 6-(2-chloro-4-(6-methylpyrazin-2-yl)phenyl)-N-methyl-8,9-dihydroimidazo[1′,2′:1,6]pyrido[2,3-d]pyrimidin-2-amine (8 mg, 40% yield, TFA salt) as a yellow solid. ¹H NMR (400 MHz, CD₃OD): δ 9.00 (s, 1H), 8.92-8.81 (m, 1H), 8.53 (s, 1H), 8.38 (d, J=1.6 Hz, 1H), 8.20 (dd, J=8.0, 1.6 Hz, 1H), 8.16 (s, 1H), 7.64 (d, J=8.0 Hz, 1H), 4.81-4.78 (m, 2H), 4.17-4.15 (m, 2H), 3.08 (s, 3H), 2.66 (s, 3H). LCMS (M+H⁺) m/z: 404.1.

### Example 26: Preparation of 6-(4-chlorophenyl)-N-(3-fluoro-4-(piperazin-1-yl)phenyl)-8,9-dihydroimidazo[1′,2′:1,6]pyrido[2,3-d]pyrimidin-2-amine (Compound 26)

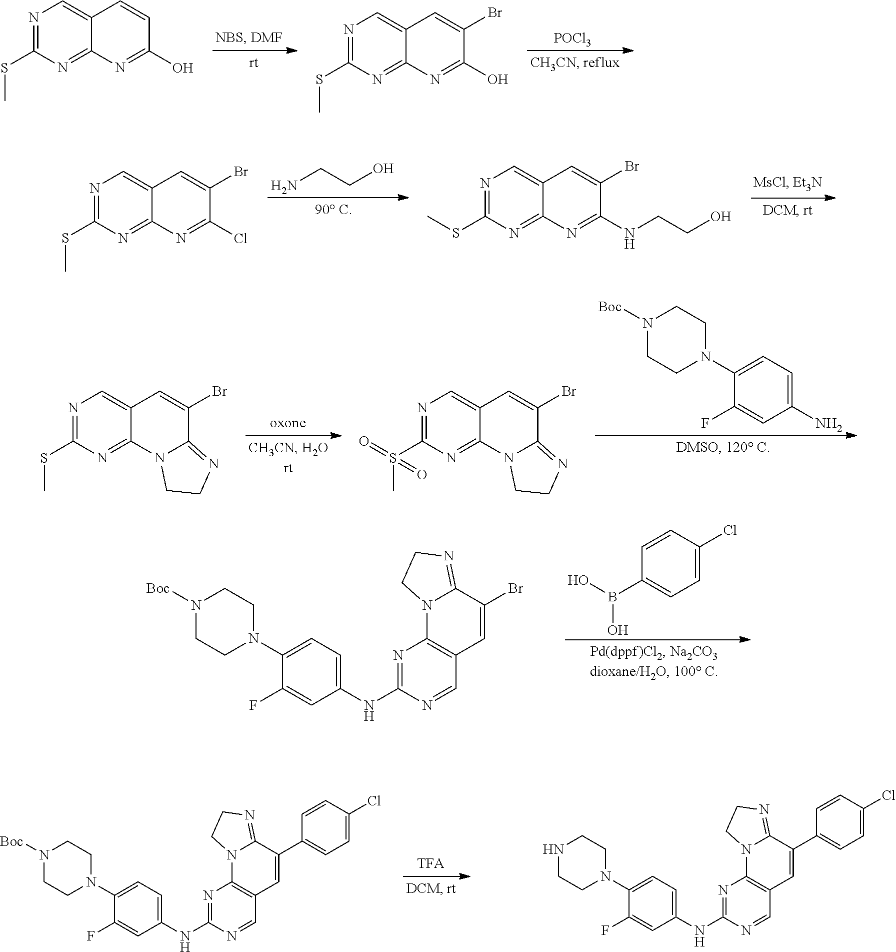

### Step 1: Synthesis of 6-bromo-2-(methylthio)pyrido[2,3-d]pyrimidin-7-ol

To a solution of 2-(methylthio)pyrido[2,3-d]pyrimidin-7-ol (8.0 g, 41.45 mmol) in DMF (200 mL) was added NBS (7.75 g, 43.52 mmol). The reaction mixture was stirred at room temperature for 16 hours. The reaction mixture was poured into water and stirred for 1 hour. The solid was filtered and dried in vacuum to give 6-bromo-2-(methylthio)pyrido[2,3-d]pyrimidin-7-ol (9.6 g, 85% yield) as a white solid. LCMS (M+H⁺) m/z: 272.0

### Step 2: Synthesis of 6-bromo-7-chloro-2-(methylthio)pyrido[2,3-d]pyrimidine

To a solution of 6-bromo-2-(methylthio)pyrido[2,3-d]pyrimidin-7-ol (9.6 g, 35.3 mmol) in CH₃CN (100 mL) was added POCl₃ (100 mL). The mixture was stirred at 100° C. for 16 hours. The mixture was concentrated in vacuum to afford 6-bromo-7-chloro-2-(methylthio)pyrido[2,3-d]pyrimidine (9.8 g, crude, 86% yield) as a white solid. LCMS (M+H⁺) m/z: 289.9

### Step 3: Synthesis of 2-((6-bromo-2-(methylthio)pyrido[2,3-d]pyrimidin-7-yl)amino)ethan-1-ol

A solution of 6-bromo-7-chloro-2-(methylthio)pyrido[2,3-d]pyrimidine (5.1 g, 17.59 mmol) and 2-aminoethanol (10 mL) was stirred at 90° C. for 2 hours under N₂. The mixture was added to water (100 mL), filtered to afford 2-((6-bromo-2-(methylthio)pyrido[2,3-d]pyrimidin-7-yl)amino)ethan-1-ol (4.2 g, 76% yield) as a yellow solid. LCMS (M+H⁺) m/z: 315.0

### Step 4: Synthesis of 6-bromo-2-(methylthio)-8,9-dihydroimidazo[1′,2′:1,6]pyrido[2,3-d]pyrimidine

To a solution of 2-((6-bromo-2-(methylthio)pyrido[2,3-d]pyrimidin-7-yl)amino)ethan-1-ol (4.2 g, 13.3 mmol) and Et₃N (4.03 g, 39.9 mmol) in DCM (100 mL) was added MsCl (3.1 g, 26.6 mmol). The reaction mixture was stirred at room temperature for 16 hours under N₂. The reaction mixture was extracted with DCM (60 mL×3), washed with brine (30 mL), dried over Na₂SO₄, concentrated in vacuum and purified by column chromatography on silica gel (DCM/CH₃OH=60:1) to give 6-bromo-2-(methylthio)-8,9-dihydroimidazo[1′,2′:1,6]pyrido[2,3-d]pyrimidine (2.3 g, 59% yield) as a yellow solid. LCMS (M+H⁺) m/z: 297.0.

### Step 5: Synthesis of 6-bromo-2-(methylsulfonyl)-8,9-dihydroimidazo[1′,2′:1,6]pyrido[2,3-d]pyrimidine

To a solution of 6-bromo-2-(methylthio)-8,9-dihydroimidazo[1′,2′:1,6]pyrido[2,3-d]pyrimidine (1.2 g, 4.04 mmol) in CH₃CN (50 mL) and H₂O (50 mL) was added oxone (3.70 g, 6.06 mmol). The reaction mixture was stirred at 30° C. under N₂ for 16 hours. The mixture was adjusted to pH=7-8 with 1N NaOH aq, extracted with DCM (100 mL×3), washed with brine (50 mL), dried over Na₂SO₄, concentrated in vacuum to give crude 6-bromo-2-(methylsulfonyl)-8,9-dihydroimidazo[1′,2′:1,6]pyrido[2,3-d]pyrimidine and without purification for the next step. LCMS (M+H⁺) m/z: 329.0.

### Step 6: Synthesis of tert-butyl 4-(4-((6-bromo-8,9-dihydroimidazo[1′,2′:1,6]pyrido[2,3-d]pyrimidin-2-yl)amino)-2-fluorophenyl)piperazine-1-carboxylate

To a solution of 6-bromo-2-(methylsulfonyl)-8,9-dihydroimidazo[1′,2′:1,6]pyrido[2,3-d]pyrimidine (360 mg, 1.09 mmol) in DMSO (10 mL) was added tert-butyl 4-(4-amino-2-fluorophenyl)piperazine-1-carboxylate (324 mg, 1.09 mmol). The reaction mixture was stirred at 120° C. for 1 hour under N₂. The mixture was added into water, extracted with EtOAc (80 mL×3), washed with brine (30 mL), dried over Na₂SO₄, concentrated in vacuum and purified by column chromatography on silica gel (DCM/CH₃OH=20:1) to afford tert-butyl 4-(4-((6-bromo-8,9-dihydroimidazo[1′,2′:1,6]pyrido[2,3-d]pyrimidin-2-yl)amino)-2-fluorophenyl)piperazine-1-carboxylate (130 mg, 22% yield) as a yellow solid. LCMS (M+H⁺) m/z: 544.1

### Step 7: Synthesis of tert-Butyl 4-(4-((6-(4-chlorophenyl)-8,9-dihydroimidazo[1′,2′:1,6]pyrido[2,3-d]pyrimidin-2-yl)amino)-2-fluorophenyl)piperazine-1-carboxylate

The mixture of tert-butyl 4-(4-((6-bromo-8,9-dihydroimidazo[1′,2′:1,6]pyrido[2,3-d]pyrimidin-2-yl)amino)-2-fluorophenyl)piperazine-1-carboxylate (60 mg, 0.11 mmol), (4-chlorophenyl)boronic acid (52 mg, 0.33 mmol), Pd(dppf)Cl₂. DCM (10.0 mg, 0.01 mmol) and Na₂CO₃ (23 mg, 0.22 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(4-((6-(4-chlorophenyl)-8,9-dihydroimidazo[1′,2′:1,6]pyrido[2,3-d]pyrimidin-2-yl)amino)-2-fluorophenyl)piperazine-1-carboxylate (45 mg, 71% yield) as a yellow solid. LCMS (M+H⁺) m/z: 576.3.

### Step 8: Synthesis of 6-(4-chlorophenyl)-N-(3-fluoro-4-(piperazin-1-yl)phenyl)-8,9-dihydroimidazo[1′,2′:1,6]pyrido[2,3-d]pyrimidin-2-amine

To a solution of tert-butyl 4-(4-((6-(4-chlorophenyl)-8,9-dihydroimidazo[1′,2′:1,6]pyrido[2,3-d]pyrimidin-2-yl)amino)-2-fluorophenyl)piperazine-1-carboxylate (45 mg, 0.078 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give 6-(4-chlorophenyl)-N-(3-fluoro-4-(piperazin-1-yl)phenyl)-8,9-dihydroimidazo[1′,2′:1,6]pyrido[2,3-d]pyrimidin-2-amine (20 mg, 50% yield, TFA salt) as a yellow solid. ¹H NMR (400 MHz, DMSO-d₆): δ 10.79 (br, 1H), 10.26 (br, 1H), 9.05 (s, 1H), 8.90 (br, 2H), 8.30 (s, 1H), 8.80 (d, J=14.0 Hz, 1H), 7.68-7.56 (m, 5H), 7.14 (t, J=9.6 Hz, 1H), 4.69-4.64 (m, 2H), 4.13-4.08 (m, 2H), 3.46-3.42 (m, 4H), 3.20-3.15 (m, 4H). LCMS (M+H⁺) m/z: 476.1.

### Example 27: Preparation of N-(3-fluoro-4-(piperazin-1-yl)phenyl)-6-phenyl-8,9-dihydroimidazo[1′,2′:1,6]pyrido[2,3-d]pyrimidin-2-amine (Compound 27)

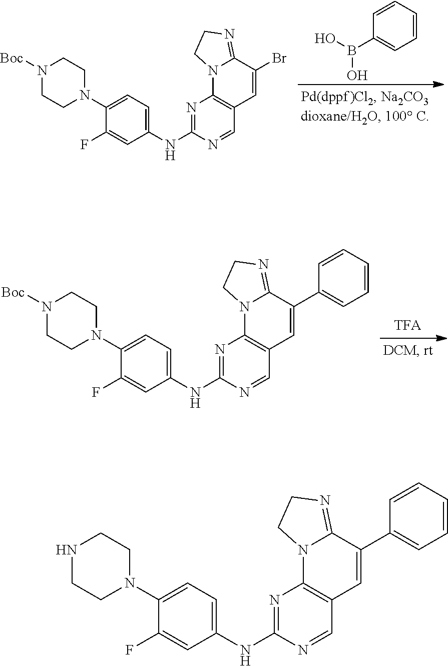 [SMILES: CC(C)(C)OC(=O)N1CCN(c2ccc(Nc3ncc4c(n3)N3CCN=C3C(Br)=C4)cc2F)CC1 ; OB(O)c1ccccc1 ; CC(C)(C)OC(=O)N1CCN(c2ccc(Nc3ncc4c(n3)N3CCN=C3C(c3ccccc3)=C4)cc2F)CC1 ; Fc1cc(Nc2ncc3c(n2)N2CCN=C2C(c2ccccc2)=C3)ccc1N1CCNCC1]

### Step 1: Synthesis of tert-butyl 4-(2-fluoro-4-((6-phenyl-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-2-fluorophenyl)piperazine-1-carboxylate (60 mg, 0.11 mmol), phenylboronic acid (40 mg, 0.33 mmol), Pd(dppf)Cl₂. DCM (18.0 mg, 0.02 mmol) and Na₂CO₃ (35 mg, 0.33 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(2-fluoro-4-((6-phenyl-8,9-dihydroimidazo[1′,2′:1,6]pyrido[2,3-d]pyrimidin-2-yl)amino)phenyl)piperazine-1-carboxylate (40 mg, 62% yield) as a yellow solid. LCMS (M+H⁺) m/z: 542.4.

### Step 2: Synthesis of N-(3-fluoro-4-(piperazin-1-yl)phenyl)-6-phenyl-8,9-dihydroimidazo[1′,2′:1,6]pyrido[2,3-d]pyrimidin-2-amine

To a solution of tert-butyl 4-(2-fluoro-4-((6-phenyl-8,9-dihydroimidazo[1′,2′:1,6]pyrido[2,3-d]pyrimidin-2-yl)amino)phenyl)piperazine-1-carboxylate (40 mg, 0.07 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(3-fluoro-4-(piperazin-1-yl)phenyl)-6-phenyl-8,9-dihydroimidazo[1′,2′:1,6]pyrido[2,3-d]pyrimidin-2-amine (20 mg, 61% yield, TFA salt) as a yellow solid. ¹H NMR (400 MHz, CD₃OD): δ 8.99 (s, 1H), 8.19 (s, 1H), 7.86-7.82 (m, 1H), 7.58-7.48 (m, 6H), 7.13 (t, J=8.8 Hz, 1H), 4.83-4.80 (m, 2H), 4.23-4.18 (m, 2H), 3.42-3.39 (m, 4H), 3.33-3.30 (m, 4H). LCMS (M+H⁺) m/z: 442.1.

### Example 28: Preparation of 6-(4-chlorophenyl)-N-(2-fluoro-4-(piperazin-1-yl)phenyl)-8,9-dihydroimidazo[1′,2′:1,6]pyrido[2,3-d]pyrimidin-2-amine (Compound 28)

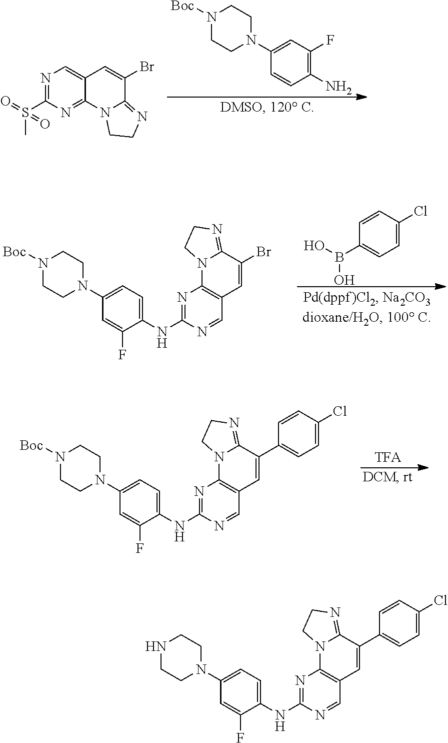 [SMILES: CS(=O)(=O)c1ncc2c(n1)N1CCN=C1C(Br)=C2 ; CC(C)(C)OC(=O)N1CCN(c2ccc(N)c(F)c2)CC1 ; CC(C)(C)OC(=O)N1CCN(c2ccc(Nc3ncc4c(n3)N3CCN=C3C(Br)=C4)c(F)c2)CC1 ; OB(O)c1ccc(Cl)cc1 ; CC(C)(C)OC(=O)N1CCN(c2ccc(Nc3ncc4c(n3)N3CCN=C3C(c3ccc(Cl)cc3)=C4)c(F)c2)CC1 ; Fc1cc(N2CCNCC2)ccc1Nc1ncc2c(n1)N1CCN=C1C(c1ccc(Cl)cc1)=C2]

### Step 1: Synthesis of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate

To a solution of 6-bromo-2-(methylsulfonyl)-8,9-dihydroimidazo[1′,2′:1,6]pyrido[2,3-d]pyrimidine (700 mg, 2.13 mmol) in DMSO (20 mL) was added tert-butyl 4-(4-amino-3-fluorophenyl)piperazine-1-carboxylate (600 mg, 2.02 mmol). The reaction mixture was stirred at 120° C. for 1 hour under N₂. The mixture was added into water, extracted with EtOAc (80 mL×3), washed with brine (30 mL), dried over Na₂SO₄, concentrated in vacuum and purified by column chromatography on silica gel (DCM/CH₃OH=60:1) to afford tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (300 mg, 25% yield) as a yellow solid. LCMS (M+H⁺) m/z: 544.1

### Step 2: Synthesis of tert-butyl 4-(4-((6-(4-chlorophenyl)-8,9-dihydroimidazo[1′,2′:1,6]pyrido[2,3-d]pyrimidin-2-yl)amino)-3-fluorophenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (60 mg, 0.11 mmol), (4-chlorophenyl)boronic acid (52 mg, 0.33 mmol), Pd(dppf)Cl₂. DCM (10.0 mg, 0.01 mmol) and Na₂CO₃ (23 mg, 0.22 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(4-((6-(4-chlorophenyl)-8,9-dihydroimidazo[1′,2′:1,6]pyrido[2,3-d]pyrimidin-2-yl)amino)-3-fluorophenyl)piperazine-1-carboxylate (30 mg, 71% yield) as a yellow solid. LCMS (M+H⁺) m/z: 576.3.

### Step 3: Synthesis of 6-(4-chlorophenyl)-N-(2-fluoro-4-(piperazin-1-yl)phenyl)-8,9-dihydroimidazo[1′,2′:1,6]pyrido[2,3-d]pyrimidin-2-amine

To a solution of tert-butyl 4-(4-((6-(4-chlorophenyl)-8,9-dihydroimidazo[1′,2′:1,6]pyrido[2,3-d]pyrimidin-2-yl)amino)-3-fluorophenyl)piperazine-1-carboxylate (30 mg, 0.05 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give 6-(4-chlorophenyl)-N-(2-fluoro-4-(piperazin-1-yl)phenyl)-8,9-dihydroimidazo[1′,2′:1,6]pyrido[2,3-d]pyrimidin-2-amine (11 mg, 48% yield, TFA salt) as a yellow solid. ¹H NMR (400 MHz, CD₃OD): δ 8.93 (br, 1H), 8.19 (s, 1H), 7.80 (br, 1H), 7.59-7.54 (m, 4H), 6.96-6.89 (m, 2H), 4.86-4.66 (m, 2H), 4.17-4.13 (m, 2H), 3.47-3.40 (m, 4H), 3.38-3.30 (m, 4H). LCMS (M+H⁺) m/z: 476.1.

### Example 29: Preparation of N-(2-fluoro-4-(piperazin-1-yl)phenyl)-6-phenyl-8,9-dihydroimidazo[1′,2′:1,6]pyrido[2,3-d]pyrimidin-2-amine (Compound 29)

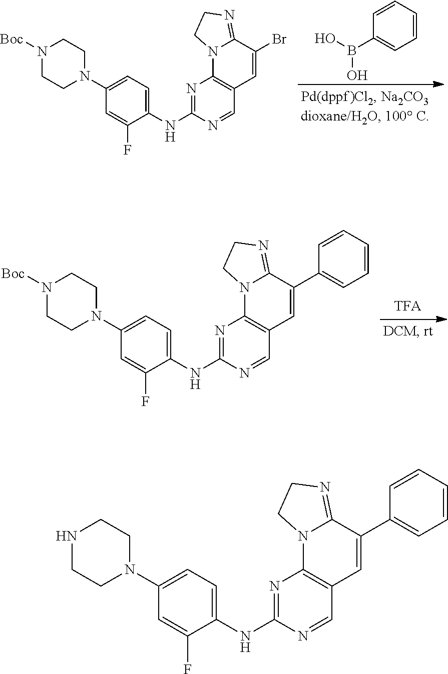 [SMILES: CC(C)(C)OC(=O)N1CCN(c2ccc(Nc3ncc4c(n3)N3CCN=C3C(Br)=C4)c(F)c2)CC1 ; OB(O)c1ccccc1 ; CC(C)(C)OC(=O)N1CCN(c2ccc(Nc3ncc4c(n3)N3CCN=C3C(c3ccccc3)=C4)c(F)c2)CC1 ; Fc1cc(N2CCNCC2)ccc1Nc1ncc2c(n1)N1CCN=C1C(c1ccccc1)=C2]

### Step 1: Synthesis of tert-butyl 4-(3-fluoro-4-((6-phenyl-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (60 mg, 0.11 mmol), phenylboronic acid (48 mg, 0.33 mmol), Pd(dppf)Cl₂. DCM (20.0 mg, 0.02 mmol) and Na₂CO₃ (23 mg, 0.22 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(3-fluoro-4-((6-phenyl-8,9-dihydroimidazo[1′,2′:1,6]pyrido[2,3-d]pyrimidin-2-yl)amino)phenyl)piperazine-1-carboxylate (35 mg, 50% yield) as a yellow solid. LCMS (M+H⁺) m/z: 542.3.

### Step 2: Synthesis of N-(2-fluoro-4-(piperazin-1-yl)phenyl)-6-phenyl-8,9-dihydroimidazo[1′,2′:1,6]pyrido[2,3-d]pyrimidin-2-amine

To a solution of tert-butyl 4-(3-fluoro-4-((6-phenyl-8,9-dihydroimidazo[1′,2′:1,6]pyrido[2,3-d]pyrimidin-2-yl)amino)phenyl)piperazine-1-carboxylate (35 mg, 0.06 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-6-phenyl-8,9-dihydroimidazo[1′,2′:1,6]pyrido[2,3-d]pyrimidin-2-amine (15 mg, 58% yield, TFA salt) as a yellow solid. ¹H NMR (400 MHz, CD₃OD): δ 8.93 (br, 1H), 8.17 (s, 1H), 7.80 (br, 1H), 7.57-7.52 (m, 5H), 6.86-6.71 (m, 2H), 4.86-4.71 (m, 2H), 4.17-4.12 (m, 2H), 3.47-3.45 (m, 4H), 3.40-3.38 (m, 4H). LCMS (M+H⁺) m/z: 442.2.

### Example 30: Preparation of N-(2-fluoro-4-(piperazin-1-yl)phenyl)-6-(o-tolyl)-8,9-dihydroimidazo[1′,2′:1,6]pyrido[2,3-d]pyrimidin-2-amine (Compound 30)

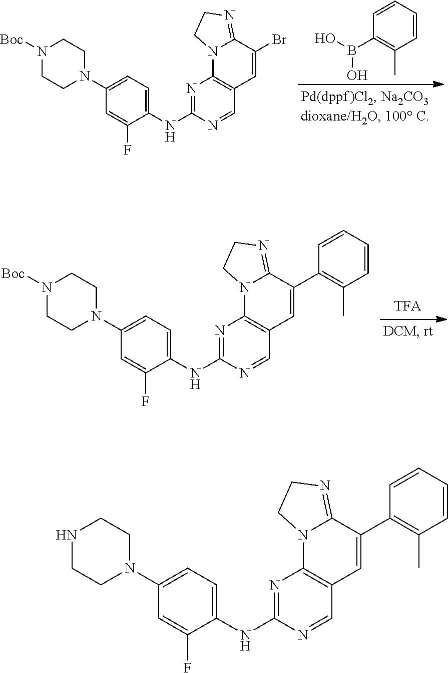

### Step 1: Synthesis of tert-butyl 4-(3-fluoro-4-((6-(o-tolyl)-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (60 mg, 0.11 mmol), o-tolylboronic acid (45 mg, 0.33 mmol), Pd(dppf)Cl₂. DCM (16.0 mg, 0.02 mmol) and Na₂CO₃ (23 mg, 0.22 mmol) in dioxane/H₂O (10 mL, v/v=5/1) at room temperature was purged and degassed with N₂ for 3 times, and then stirred at 100° C. for 16 hours. The reaction mixture was concentrated to remove solvent and the residue was purified by column chromatography on silica gel (DCM/CH₃OH=30:1) to afford tert-butyl 4-(3-fluoro-4-((6-(o-tolyl)-8,9-dihydroimidazo[1′,2′:1,6]pyrido[2,3-d]pyrimidin-2-yl)amino)phenyl)piperazine-1-carboxylate (35 mg, 57% yield) as a yellow solid. LCMS (M+H⁺) m/z: 556.4.

### Step 2: Synthesis of N-(2-fluoro-4-(piperazin-1-yl)phenyl)-6-(o-tolyl)-8,9-dihydroimidazo[1′,2′:1,6]pyrido[2,3-d]pyrimidin-2-amine

To a solution of tert-butyl 4-(3-fluoro-4-((6-(o-tolyl)-8,9-dihydroimidazo[1′,2′:1,6]pyrido[2,3-d]pyrimidin-2-yl)amino)phenyl)piperazine-1-carboxylate (35 mg, 0.06 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-6-(o-tolyl)-8,9-dihydroimidazo[1′,2′:1,6]pyrido[2,3-d]pyrimidin-2-amine (16 mg, 56% yield, TFA salt) as a yellow solid. ¹H NMR (400 MHz, DMSO-d₆): δ 10.03 (br, 2H), 8.92 (br, 3H), 8.06 (br, 1H), 7.43-7.39 (m, 3H), 7.35 (t, J=7.2 Hz, 1H), 7.28 (dd, J=7.2 Hz, 1H), 6.97 (dd, J=13.6, 2.4 Hz, 1H), 6.85 (dd, J=8.8, 2.4 Hz, 1H), 4.50-4.47 (m, 2H), 4.01-3.99 (m, 2H), 3.42-3.39 (m, 4H), 3.25-3.23 (m, 4H), 2.23 (s, 3H). LCMS (M+H⁺) m/z: 456.2.

### Example 31: Preparation of N-(2-fluoro-4-(piperazin-1-yl)phenyl)-6-(2-methoxyphenyl)-8,9-dihydroimidazo[1′,2′:1,6]pyrido[2,3-d]pyrimidin-2-amine (Compound 31)

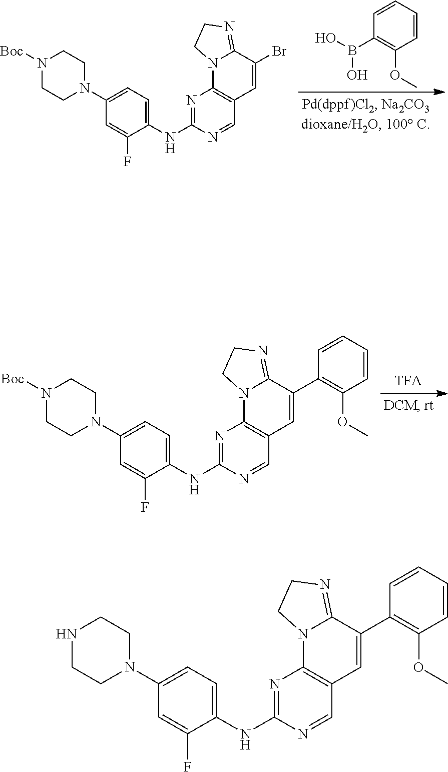 [SMILES: CC(C)(C)OC(=O)N1CCN(c2ccc(Nc3ncc4c(n3)N3CCN=C3C(Br)=C4)c(F)c2)CC1 ; COc1ccccc1B(O)O ; COc1ccccc1C1=Cc2cnc(Nc3ccc(N4CCN(C(=O)OC(C)(C)C)CC4)cc3F)nc2N2CCN=C12 ; COc1ccccc1C1=Cc2cnc(Nc3ccc(N4CCNCC4)cc3F)nc2N2CCN=C12]

### Step 1: Synthesis of tert-butyl 4-(3-fluoro-4-((6-(2-methoxyphenyl)-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (60 mg, 0.11 mmol), (2-methoxyphenyl)boronic acid (50 mg, 0.33 mmol), Pd(dppf)Cl₂. DCM (16.0 mg, 0.02 mmol) and Na₂CO₃ (23 mg, 0.22 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(3-fluoro-4-((6-(2-methoxyphenyl)-8,9-dihydroimidazo[1′,2′:1,6]pyrido[2,3-d]pyrimidin-2-yl)amino)phenyl)piperazine-1-carboxylate (35 mg, 58% yield) as a yellow solid. LCMS (M+H⁺) m/z: 572.3.

### Step 2: Synthesis of N-(2-fluoro-4-(piperazin-1-yl)phenyl)-6-(2-methoxyphenyl)-8,9-dihydroimidazo[1′,2′:1,6]pyrido[2,3-d]pyrimidin-2-amine

To a solution of tert-butyl 4-(3-fluoro-4-((6-(2-methoxyphenyl)-8,9-dihydroimidazo[1′,2′:1,6]pyrido[2,3-d]pyrimidin-2-yl)amino)phenyl)piperazine-1-carboxylate (35 mg, 0.06 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-6-(2-methoxyphenyl)-8,9-dihydroimidazo[1′,2′:1,6]pyrido[2,3-d]pyrimidin-2-amine (23 mg, 56% yield, TFA salt) as a yellow solid. ¹H NMR (400 MHz, DMSO-d₆): δ 10.12 (br, 1H), 9.87 (br, 1H), 8.97-8.90 (m, 3H), 8.18 (s, 1H), 7.55-7.51 (m, 1H), 7.34 (dd, J=7.6, 1.6 Hz, 1H), 7.21 (d, J=8.4 Hz, 1H), 7.12 (t, J=7.6 Hz, 1H), 6.98 (dd, J=13.6, 1.6 Hz, 1H), 6.85 (dd, J=8.4, 2.0 Hz, 1H), 4.51-4.49 (m, 2H), 4.05-3.99 (m, 2H), 3.81 (s, 3H), 3.42-3.39 (m, 4H), 3.25-3.23 (m, 4H). LCMS (M+H⁺) m/z: 472.2.

### Example 32: Preparation of N-(2-fluoro-4-(piperazin-1-yl)phenyl)-6-(pyridin-2-yl)-8,9-dihydroimidazo[1′,2′:1,6]pyrido[2,3-d]pyrimidin-2-amine (Compound 32)

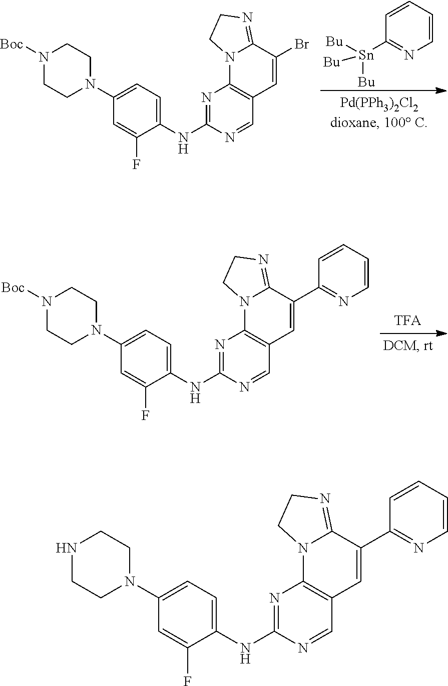

### Step 1: Synthesis of tert-butyl 4-(3-fluoro-4-((6-(pyridin-2-yl)-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (60 mg, 0.11 mmol), 2-(tributylstannyl) pyridine (122 mg, 0.33 mmol), Pd(PPh₃)₂Cl₂. (16.0 mg, 0.023 mmol) in dioxane (10 mL) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(3-fluoro-4-((6-(pyridin-2-yl)-8,9-dihydroimidazo[1′,2′:1,6]pyrido[2,3-d]pyrimidin-2-yl)amino)phenyl)piperazine-1-carboxylate (30 mg, 35% yield) as a yellow solid. LCMS (M+H⁺) m/z: 543.3.

### Step 2: Synthesis of N-(2-fluoro-4-(piperazin-1-yl)phenyl)-6-(pyridin-2-yl)-8,9-dihydroimidazo[1′,2′:1,6]pyrido[2,3-d]pyrimidin-2-amine

To a solution of tert-butyl 4-(3-fluoro-4-((6-(pyridin-2-yl)-8,9-dihydroimidazo[1′,2′:1,6]pyrido[2,3-d]pyrimidin-2-yl)amino)phenyl)piperazine-1-carboxylate (38 mg, 0.07 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-6-(pyridin-2-yl)-8,9-dihydroimidazo[1′,2′:1,6]pyrido[2,3-d]pyrimidin-2-amine (12 mg, 37% yield, TFA salt) as a yellow solid. ¹H NMR (400 MHz, DMSO-d₆): δ 10.96-10.83 (m, 1H), 10.30-10.25 (m, 1H), 9.08 (s, 1H), 9.07-8.92 (m, 3H), 8.70 (d, J=4.0 Hz, 1H), 8.17 (d, J=8.0 Hz, 1H), 8.09-8.07 (m, 1H), 7.53-7.32 (m, 2H), 7.02-6.98 (m, 1H), 6.87-6.85 (m, 1H), 4.62-4.41 (m, 2H), 4.22-4.18 (m, 2H), 3.50-3.46 (m, 4H), 3.41-3.39 (m, 4H). LCMS (M+H⁺) m/z: 443.2.

### Example 33: Preparation of N-(2-fluoro-4-(piperazin-1-yl)phenyl)-6-(thiazol-4-yl)-8,9-dihydroimidazo[1′,2′:1,6]pyrido[2,3-d]pyrimidin-2-amine (Compound 33)

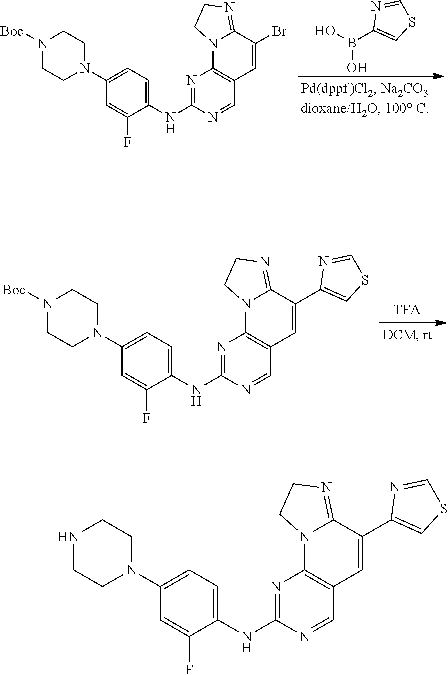 [SMILES: CC(C)(C)OC(=O)N1CCN(c2ccc(Nc3ncc4c(n3)N3CCN=C3C(Br)=C4)c(F)c2)CC1 ; OB(O)c1cscn1 ; CC(C)(C)OC(=O)N1CCN(c2ccc(Nc3ncc4c(n3)N3CCN=C3C(c3cscn3)=C4)c(F)c2)CC1 ; Fc1cc(N2CCNCC2)ccc1Nc1ncc2c(n1)N1CCN=C1C(c1cscn1)=C2]

### Step 1: Synthesis of tert-butyl 4-(3-fluoro-4-((6-(thiazol-4-yl)-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (50 mg, 0.10 mmol), thiazol-4-ylboronic acid (30 mg, 0.32 mmol), Pd(dppf)Cl₂. DCM (18.0 mg, 0.02 mmol) and Na₂CO₃ (35 mg, 0.33 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(3-fluoro-4-((6-(thiazol-4-yl)-8,9-dihydroimidazo[1′,2′:1,6]pyrido[2,3-d]pyrimidin-2-yl)amino)phenyl)piperazine-1-carboxylate (30 mg, 20% yield) as a yellow solid. LCMS (M+H⁺) m/z: 549.2.

### Step 2: Synthesis of N-(2-fluoro-4-(piperazin-1-yl)phenyl)-6-(thiazol-4-yl)-8,9-dihydroimidazo[1′,2′:1,6]pyrido[2,3-d]pyrimidin-2-amine

To a solution of tert-butyl 4-(3-fluoro-4-((6-(thiazol-4-yl)-8,9-dihydroimidazo[1′,2′:1,6]pyrido[2,3-d]pyrimidin-2-yl)amino)phenyl)piperazine-1-carboxylate (30 mg, 0.06 mmol) in DCM (10 mL), was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-6-(thiazol-4-yl)-8,9-dihydroimidazo[1′,2′:1,6]pyrido[2,3-d]pyrimidin-2-amine (8 mg, 25% yield, TFA salt) as a yellow solid. ¹H NMR (400 MHz, CD₃OD): δ 9.20 (s, 1H), 8.96 (br, 1H), 8.77 (s, 1H), 8.27 (s, 1H), 7.86 (br, 1H), 6.96-6.89 (m, 2H), 4.88-4.68 (m, 2H), 4.30-4.25 (m, 2H), 3.48-3.45 (m, 4H), 3.40-3.30 (m, 4H). LCMS (M+H⁺) m/z: 449.1.

### Example 34: Preparation of N-(2-fluoro-4-(piperazin-1-yl)phenyl)-6-(pyridin-4-yl)-8,9-dihydroimidazo[1′,2′:1,6]pyrido[2,3-d]pyrimidin-2-amine (Compound 34)

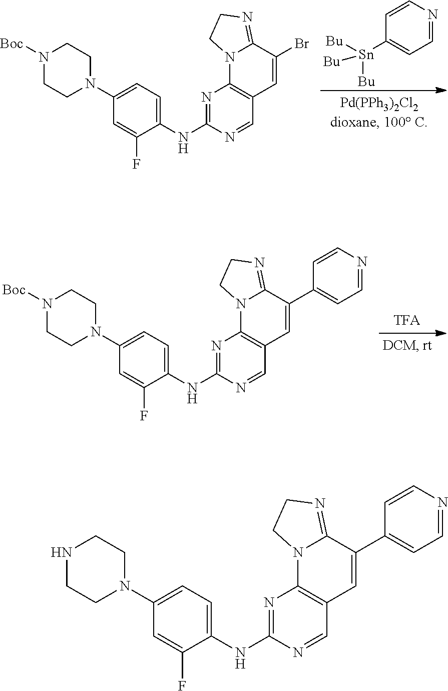

### Step 1: Synthesis of tert-butyl 4-(3-fluoro-4-((6-(pyridin-4-yl)-8,9-dihydroimidazo[1′,2′:1,6]pyrido[2,3-d]pyrimidin-2-yl)amino)phenyl)piperazine-1-carboxylate

A mixture of tert-butyl 4-(4-((6-bromo-8,9-dihydroimidazo[1′,2′:1,6]pyrido[2,3-d]pyrimidin-2-yl)amino)-3-fluorophenyl)piperazine-1-carboxylate (60 mg, 0.11 mmol), 4-(tributylstannyl) pyridine (122 mg, 0.33 mmol), Pd(PPh₃)₂Cl₂. (16.0 mg, 0.023 mmol) in dioxane (10 mL) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(3-fluoro-4-((6-(pyridin-4-yl)-8,9-dihydroimidazo[1′,2′:1,6]pyrido[2,3-d]pyrimidin-2-yl)amino)phenyl)piperazine-1-carboxylate (15 mg, 15% yield) as a yellow solid LCMS (M+H⁺) m/z: 543.2.

### Step 2: Synthesis of N-(2-fluoro-4-(piperazin-1-yl)phenyl)-6-(pyridin-4-yl)-8,9-dihydroimidazo[1′,2′:1,6]pyrido[2,3-d]pyrimidin-2-amine

To a solution of tert-butyl 4-(3-fluoro-4-((6-(pyridin-4-yl)-8,9-dihydroimidazo[1′,2′:1,6]pyrido[2,3-d]pyrimidin-2-yl)amino)phenyl)piperazine-1-carboxylate (15 mg, 0.03 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-fluoro-4-(piperazin-1-yl)phenyl)-6-(pyridin-4-yl)-8,9-dihydroimidazo[1′,2′:1,6]pyrido[2,3-d]pyrimidin-2-amine (12 mg, 30% yield, TFA salt) as a yellow solid. ¹H NMR (400 MHz, CD₃OD): δ 8.99 (s, 1H), 8.87 (s, 2H), 8.41 (s, 1H), 7.87-7.85 (m, 3H), 6.96-6.89 (m, 2H), 4.72-4.65 (m, 2H), 4.19-4.16 (m, 2H), 3.48-3.46 (m, 4H), 3.40-3.37 (m, 4H). LCMS (M+H⁺) m/z: 443.2.

### Example 35: Preparation of 6-(2,4-dichlorophenyl)-N-(2-methoxy-6-(piperazin-1-yl)pyridin-3-yl)-8,9-dihydroimidazo[1′,2′:1,6]pyrido[2,3-d]pyrimidin-2-amine (Compound 35)

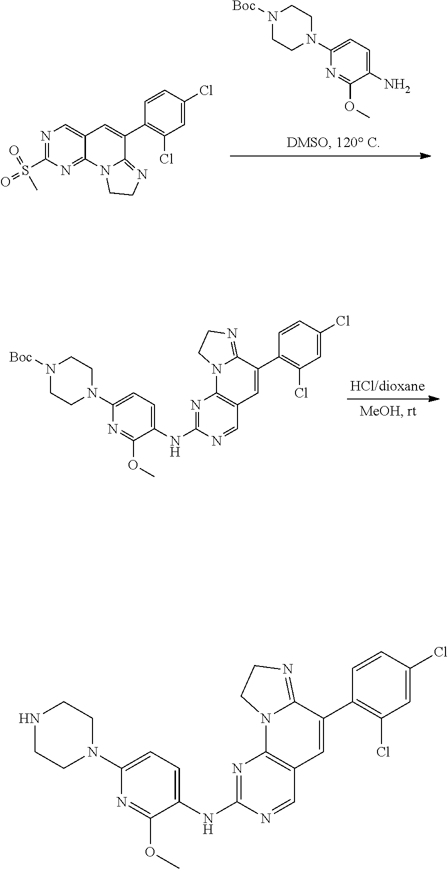 [SMILES: CS(=O)(=O)c1ncc2c(n1)N1CCN=C1C(c1ccc(Cl)cc1Cl)=C2 ; COc1nc(N2CCN(C(=O)OC(C)(C)C)CC2)ccc1N ; COc1nc(N2CCN(C(=O)OC(C)(C)C)CC2)ccc1Nc1ncc2c(n1)N1CCN=C1C(c1ccc(Cl)cc1Cl)=C2 ; COc1nc(N2CCNCC2)ccc1Nc1ncc2c(n1)N1CCN=C1C(c1ccc(Cl)cc1Cl)=C2]

### Step 1: Synthesis of tert-butyl 4-(5-((6-(2,4-dichlorophenyl)-8,9-dihydroimidazo[1′,2′:1,6]pyrido[2,3-d]pyrimidin-2-yl)amino)-6-methoxypyridin-2-yl)piperazine-1-carboxylate

To a solution of 6-(2,4-dichlorophenyl)-2-(methylsulfonyl)-8,9-dihydroimidazo[1′,2′:1,6]pyrido[2,3-d]pyrimidine (80 mg, 0.20 mmol) in DMSO (3 mL) was added tert-butyl 4-(5-amino-6-methoxypyridin-2-yl)piperazine-1-carboxylate (62 mg, 0.20 mmol). The reaction mixture was stirred at 120° C. for 2 hours under N₂. The mixture was added to water, extracted with EtOAc (10 mL×3), washed with brine (50 mL), dried over Na₂SO₄, concentrated and purified by pre-TLC (EtOAc) to afford tert-butyl 4-(5-((6-(2,4-dichlorophenyl)-8,9-dihydroimidazo[1′,2′:1,6]pyrido[2,3-d]pyrimidin-2-yl)amino)-6-methoxypyridin-2-yl)piperazine-1-carboxylate (25 mg, 19% yield) as a yellow solid. LCMS (M+H⁺) m/z: 623.3.

### Step 2: Synthesis of 6-(2,4-dichlorophenyl)-N-(2-methoxy-6-(piperazin-1-yl)pyridin-3-yl)-8,9-dihydroimidazo[1′,2′:1,6]pyrido[2,3-d]pyrimidin-2-amine

To a solution of tert-butyl 4-(5-((6-(2,4-dichlorophenyl)-8,9-dihydroimidazo[1′,2′:1,6]pyrido[2,3-d]pyrimidin-2-yl)amino)-6-methoxypyridin-2-yl)piperazine-1-carboxylate (25 mg, 0.040 mmol) in MeOH (1 mL), was added HCl/dioxane (2 mL, 3M). The mixture was stirred at room temperature for 2 hours under N₂. The residue was concentrated and purified by Prep-HPLC (0.1% TFA/CH₃CN/H₂O) to give 6-(2,4-dichlorophenyl)-N-(2-methoxy-6-(piperazin-1-yl)pyridin-3-yl)-8,9-dihydroimidazo[1′,2′:1,6]pyrido[2,3-d]pyrimidin-2-amine (13.4 mg, 50% yield, TFA salt) as a white solid. ¹H NMR (400 MHz, DMSO-d₆): δ 8.99-8.88 (m, 1H), 8.21 (s, 1H), 7.97-7.95 (m, 1H), 7.87 (s, 1H), 7.63 (d, J=8.4 Hz, 1H), 7.53 (d, J=8.4 Hz, 1H), 6.50 (d, J=8.4 Hz, 1H), 4.76-4.40 (m, 2H), 4.10-3.96 (m, 2H), 3.88 (s, 3H), 3.76-3.69 (m, 4H), 3.26-3.20 (m, 4H). LCMS (M+H⁺) m/z: 523.3.

### Example 36: Preparation of 6-(4-chlorophenyl)-N-(2-methoxy-6-(piperazin-1-yl)pyridin-3-yl)-8,9-dihydroimidazo[1′,2′:1,6]pyrido[2,3-d]pyrimidin-2-amine (Compound 36)

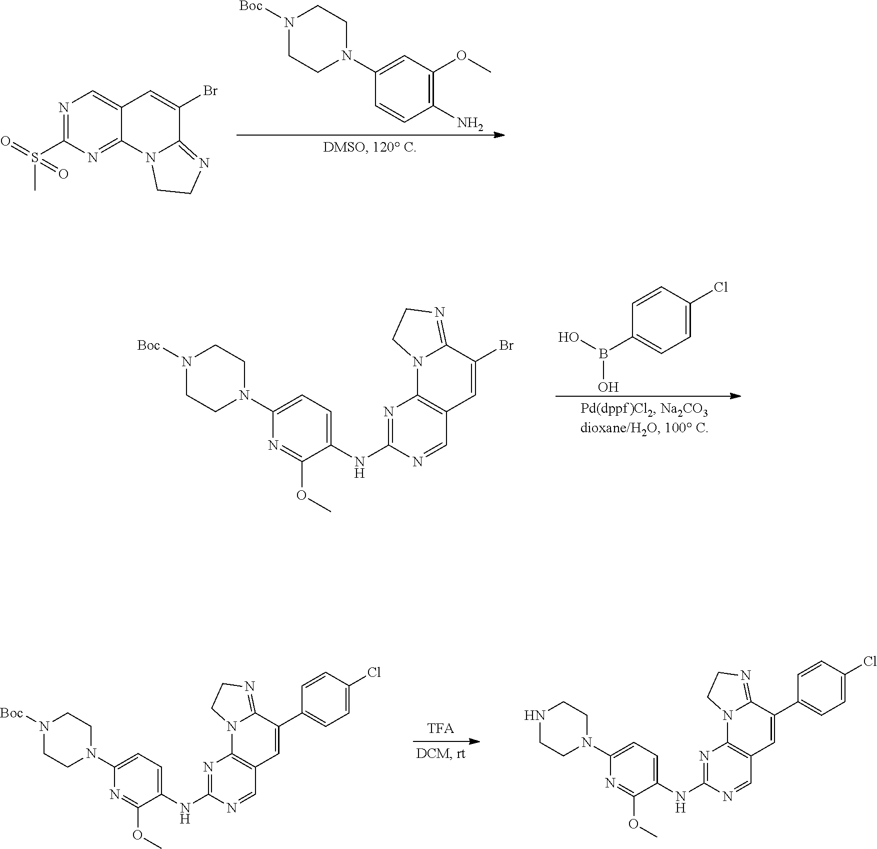 [SMILES: CS(=O)(=O)c1ncc2c(n1)N1CCN=C1C(Br)=C2 ; COc1cc(N2CCN(C(=O)OC(C)(C)C)CC2)ccc1N ; COc1nc(N2CCN(C(=O)OC(C)(C)C)CC2)ccc1Nc1ncc2c(n1)N1CCN=C1C(Br)=C2 ; OB(O)c1ccc(Cl)cc1 ; COc1nc(N2CCN(C(=O)OC(C)(C)C)CC2)ccc1Nc1ncc2c(n1)N1CCN=C1C(c1ccc(Cl)cc1)=C2 ; COc1nc(N2CCNCC2)ccc1Nc1ncc2c(n1)N1CCN=C1C(c1ccc(Cl)cc1)=C2]

### Step 1: Synthesis of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate

To a solution of 6-bromo-2-(methylsulfonyl)-8,9-dihydroimidazo[1′,2′:1,6]pyrido[2,3-d]pyrimidine (300 mg, 0.91 mmol) in DMSO (10 mL) was added tert-butyl 4-(5-amino-6-methoxypyridin-2-yl)piperazine-1-carboxylate (280 mg, 0.91 mmol). The reaction mixture was stirred at 120° C. for 1 hour under N₂. The mixture was added into water, extracted with EtOAc (80 mL×3), washed with brine (30 mL), dried over Na₂SO₄, concentrated in vacuum and purified by column chromatography on silica gel (DCM/CH₃OH=60:1) to afford tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (200 mg, 40% yield) as a yellow solid. LCMS (M+H⁺-Boc) m/z: 457.2

### Step 2: Synthesis of tert-butyl 4-(5-((6-(4-chlorophenyl)-8,9-dihydroimidazo[1′,2′:1,6]pyrido[2,3-d]pyrimidin-2-yl)amino)-6-methoxypyridin-2-yl)piperazine-1-carboxylate

A mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (60 mg, 0.11 mmol), (4-chlorophenyl)boronic acid (50 mg, 0.33 mmol), Pd(dppf)Cl₂. DCM (18.0 mg, 0.02 mmol) and Na₂CO₃ (35 mg, 0.33 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(5-((6-(4-chlorophenyl)-8,9-dihydroimidazo[1′,2′:1,6]pyrido[2,3-d]pyrimidin-2-yl)amino)-6-methoxypyridin-2-yl)piperazine-1-carboxylate (40 mg, 62% yield) as a yellow solid. LCMS (M+H⁺) m/z: 589.3.

### Step 3: Synthesis of 6-(4-chlorophenyl)-N-(2-methoxy-6-(piperazin-1-yl)pyridin-3-yl)-8,9-dihydroimidazo[1′,2′:1,6]pyrido[2,3-d]pyrimidin-2-amine

To a solution of tert-butyl 4-(5-((6-(4-chlorophenyl)-8,9-dihydroimidazo[1′,2′:1,6]pyrido[2,3-d]pyrimidin-2-yl)amino)-6-methoxypyridin-2-yl)piperazine-1-carboxylate (40 mg, 0.07 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give 6-(4-chlorophenyl)-N-(2-methoxy-6-(piperazin-1-yl)pyridin-3-yl)-8,9-dihydroimidazo[1′,2′:1,6]pyrido[2,3-d]pyrimidin-2-amine (25 mg, 48% yield, TFA salt) as a yellow solid. ¹H NMR (400 MHz, DMSO-d₆): δ 10.11 (br, 1H), 9.69 (s, 1H), 8.97-8.90 (m, 3H), 8.26 (s, 1H), 7.90 (br, 1H), 7.65 (dd, J=6.8, 2.0 Hz, 2H), 7.58 (dd, J=6.8, 2.0 Hz, 2H), 6.49 (d, J=8.4 Hz, 1H), 4.51-4.46 (m, 2H), 4.02-3.98 (m, 2H), 3.86 (s, 3H), 3.72-3.47 (m, 4H), 3.25-3.23 (m, 4H). LCMS (M+H⁺) m/z: 489.1.

### Example 37: Preparation of N-(2-methoxy-6-(piperazin-1-yl)pyridin-3-yl)-6-phenyl-8,9-dihydroimidazo[1′,2′:1,6]pyrido[2,3-d]pyrimidin-2-amine (Compound 37)

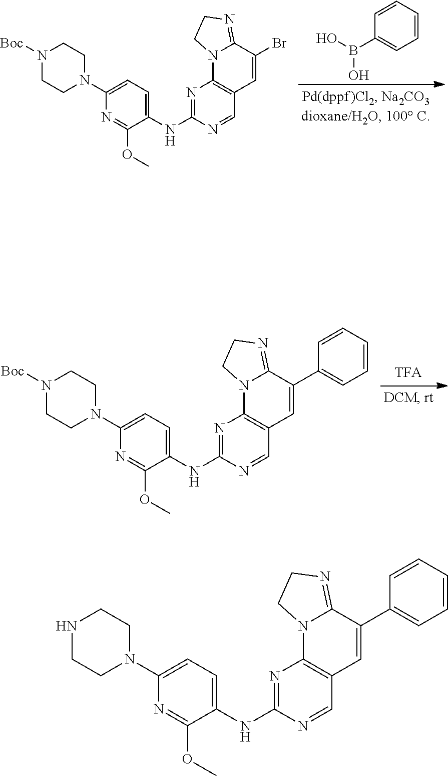 [SMILES: COc1nc(N2CCN(C(=O)OC(C)(C)C)CC2)ccc1Nc1ncc2c(n1)N1CCN=C1C(Br)=C2 ; OB(O)c1ccccc1 ; COc1nc(N2CCN(C(=O)OC(C)(C)C)CC2)ccc1Nc1ncc2c(n1)N1CCN=C1C(c1ccccc1)=C2 ; COc1nc(N2CCNCC2)ccc1Nc1ncc2c(n1)N1CCN=C1C(c1ccccc1)=C2]

### Step 1: Synthesis of tert-butyl 4-(6-methoxy-5-((6-phenyl-8,9-dihydroimidazo[1′,2′:1,6]pyrido[2,3-d]pyrimidin-2-yl)amino)pyridin-2-yl)piperazine-1-carboxylate

A mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (60 mg, 0.11 mmol), (phenylboronic acid (27 mg, 0.22 mmol), Pd(dppf)Cl₂. DCM (18.0 mg, 0.02 mmol) and Na₂CO₃ (35 mg, 0.33 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(6-methoxy-5-((6-phenyl-8,9-dihydroimidazo[1′,2′:1,6]pyrido[2,3-d]pyrimidin-2-yl)amino)pyridin-2-yl)piperazine-1-carboxylate (38 mg, 62% yield) as a yellow solid. LCMS (M+H⁺) m/z: 555.4.

### Step 2: Synthesis of N-(2-methoxy-6-(piperazin-1-yl)pyridin-3-yl)-6-phenyl-8,9-dihydroimidazo[1′,2′:1,6]pyrido[2,3-d]pyrimidin-2-amine

To a solution of tert-butyl 4-(6-methoxy-5-((6-phenyl-8,9-dihydroimidazo[1′,2′:1,6]pyrido[2,3-d]pyrimidin-2-yl)amino)pyridin-2-yl)piperazine-1-carboxylate (45 mg, 0.08 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-methoxy-6-(piperazin-1-yl)pyridin-3-yl)-6-phenyl-8,9-dihydroimidazo[1′,2′:1,6]pyrido[2,3-d]pyrimidin-2-amine (30 mg, 67% yield, TFA salt) as a yellow solid. ¹H NMR (400 MHz, DMSO-d₆): δ 10.06 (br, 1H), 9.65 (s, 1H), 8.97-8.90 (m, 3H), 8.26 (s, 1H), 7.90 (br, 1H), 7.60-7.51 (m, 5H), 6.49 (d, J=8.8 Hz, 1H), 4.63-4.46 (m, 2H), 4.03-3.99 (m, 2H), 3.86 (s, 3H), 3.51-3.45 (m, 4H), 3.25-3.23 (m, 4H). LCMS (M+H⁺) m/z: 455.2.

### Example 38: Preparation of N-(2-methoxy-6-(piperazin-1-yl)pyridin-3-yl)-6-(p-tolyl)-8,9-dihydroimidazo[1′,2′:1,6]pyrido[2,3-d]pyrimidin-2-amine (Compound 38)

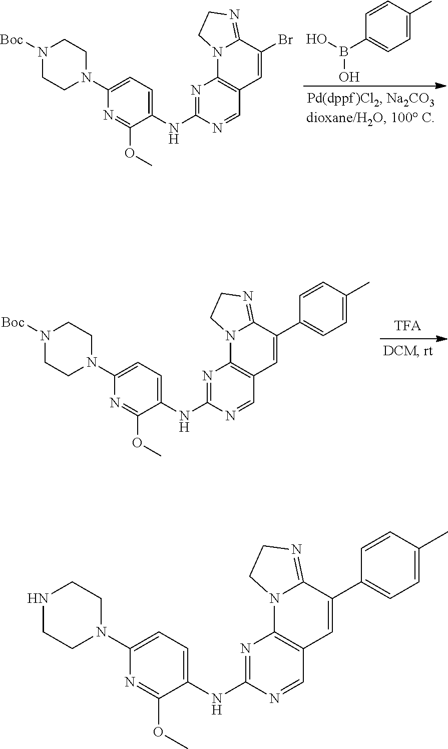

### Step 1: Synthesis of tert-butyl 4-(6-methoxy-5-((6-(p-tolyl)-8,9-dihydroimidazo[1′,2′:1,6]pyrido[2,3-d]pyrimidin-2-yl)amino)pyridin-2-yl)piperazine-1-carboxylate

A mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (60 mg, 0.11 mmol), p-tolylboronic acid (50 mg, 0.32 mmol), Pd(dppf)Cl₂. DCM (18.0 mg, 0.02 mmol) and Na₂CO₃ (35 mg, 0.33 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(6-methoxy-5-((6-(p-tolyl)-8,9-dihydroimidazo[1′,2′:1,6]pyrido[2,3-d]pyrimidin-2-yl)amino)pyridin-2-yl)piperazine-1-carboxylate (48 mg, 62% yield) as a yellow solid. LCMS (M+H⁺) m/z: 569.4.

### Step 2: Synthesis of N-(2-Methoxy-6-(piperazin-1-yl)pyridin-3-yl)-6-(p-tolyl)-8,9-dihydroimidazo[1′,2′:1,6]pyrido[2,3-d]pyrimidin-2-amine

To a solution of tert-butyl 4-(6-methoxy-5-((6-(p-tolyl)-8,9-dihydroimidazo[1′,2′:1,6]pyrido[2,3-d]pyrimidin-2-yl)amino)pyridin-2-yl)piperazine-1-carboxylate (48 mg, 0.09 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-methoxy-6-(piperazin-1-yl)pyridin-3-yl)-6-(p-tolyl)-8,9-dihydroimidazo[1′,2′:1,6]pyrido[2,3-d]pyrimidin-2-amine (30 mg, 57% yield, TFA salt) as a yellow solid. ¹H NMR (400 MHz, DMSO-d₆): δ 10.05 (br, 1H), 9.63 (s, 1H), 9.08-8.98 (m, 3H), 8.22 (s, 1H), 7.91 (br, 1H), 7.45 (d, J=8.4 Hz, 2H), 7.38 (d, J=8.4 Hz, 2H), 6.48 (d, J=8.4 Hz, 1H), 4.48-4.46 (m, 2H), 4.03-3.99 (m, 2H), 3.86 (s, 3H), 3.73-3.70 (m, 4H), 3.25-3.23 (m, 4H), 2.40 (s, 3H). LCMS (M+H⁺) m/z: 469.2.

### Example 39: Preparation of N-(2-methoxy-6-(piperazin-1-yl)pyridin-3-yl)-6-(pyridin-2-yl)-8,9-dihydroimidazo[1′,2′:1,6]pyrido[2,3-d]pyrimidin-2-amine (Compound 39)

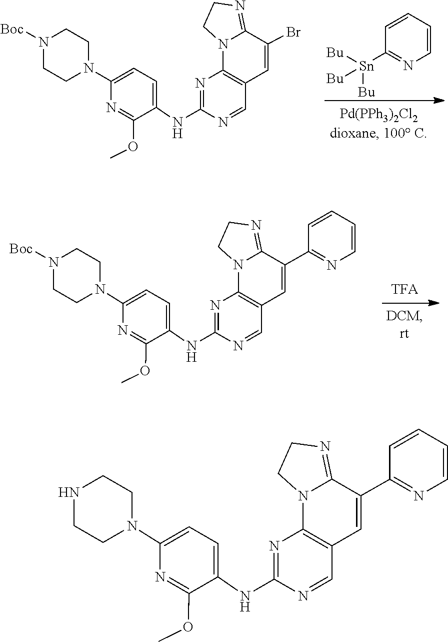

### Step 1: Synthesis of tert-butyl 4-(6-methoxy-5-((6-(pyridin-2-yl)-8,9-dihydroimidazo[1′,2′:1,6]pyrido[2,3-d]pyrimidin-2-yl)amino)pyridin-2-yl)piperazine-1-carboxylate

A mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (45 mg, 0.08 mmol), 2-(tributylstannyl) pyridine (90 mg, 0.24 mmol), Pd(PPh₃)₂Cl₂. (10.0 mg, 0.014 mmol) in dioxane (10 mL) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(6-methoxy-5-((6-(pyridin-2-yl)-8,9-dihydroimidazo[1′,2′:1,6]pyrido[2,3-d]pyrimidin-2-yl)amino)pyridin-2-yl)piperazine-1-carboxylate (20 mg, 42% yield) as a yellow solid. LCMS (M+H⁺) m/z: 556.2

### Step 2: Synthesis of N-(2-methoxy-6-(piperazin-1-yl)pyridin-3-yl)-6-(pyridin-2-yl)-8,9-dihydroimidazo[1′,2′:1,6]pyrido[2,3-d]pyrimidin-2-amine

To a solution of tert-butyl 4-(6-methoxy-5-((6-(pyridin-2-yl)-8,9-dihydroimidazo[1′,2′:1,6]pyrido[2,3-d]pyrimidin-2-yl)amino)pyridin-2-yl)piperazine-1-carboxylate (20 mg, 0.04 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-methoxy-6-(piperazin-1-yl)pyridin-3-yl)-6-(pyridin-2-yl)-8,9-dihydroimidazo[1′,2′:1,6]pyrido[2,3-d]pyrimidin-2-amine (2 mg, 7% yield, TFA salt) as a yellow solid. ¹H NMR (400 MHz, CD₃OD): δ 8.94-8.91 (m, 2H), 8.71-8.70 (m, 1H), 8.34 (br, 1H), 8.17-8.15 (m, 1H), 8.00-8.02 (m, 1H), 7.48-7.45 (m, 1H), 6.52-6.49 (m, 1H), 4.72-4.70 (m, 2H), 4.33-4.29 (m, 2H), 3.98 (s, 3H), 3.89-3.82 (m, 4H), 3.35-3.31 (m, 4H). LCMS (M+H⁺) m/z: 456.2.

### Example 40: Preparation of N-(2-methoxy-6-(piperazin-1-yl)pyridin-3-yl)-6-(pyridin-3-ylethynyl)-8,9-dihydroimidazo[1′,2′:1,6]pyrido[2,3-d]pyrimidin-2-amine (Compound 40)

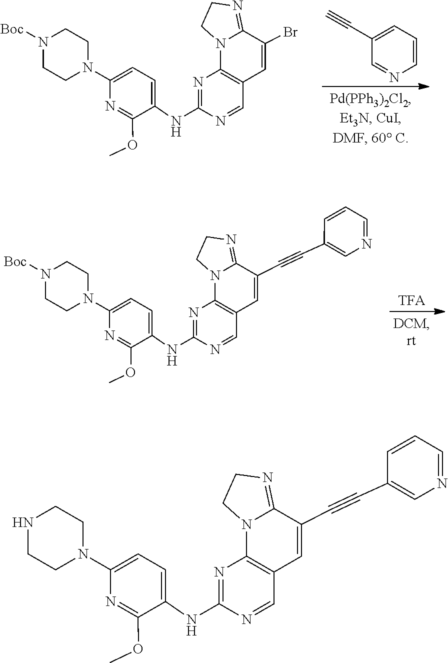

### Step 1: Synthesis of tert-butyl 4-(6-methoxy-5-((6-(pyridin-3-ylethynyl)-8,9-dihydroimidazo[1′,2′:1,6]pyrido[2,3-d]pyrimidin-2-yl)amino)pyridin-2-yl)piperazine-1-carboxylate

A mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (60 mg, 0.11 mmol), 3-ethynylpyridine (13 mg, 0.13 mmol), Pd(PPh₃)₂Cl₂. (10.0 mg, 0.014 mmol), Et₃N (0.5 mL), CuI (5 mg, 0.03 mmol) in DMF (10 mL) at room temperature was purged and degassed with N₂ for 3 times, and then stirred at 60° C. for 16 hours. The reaction mixture was concentrated to remove the solvent and the residue was purified by column chromatography on silica gel (DCM/CH₃OH=30:1) to afford tert-butyl 4-(6-methoxy-5-((6-(pyridin-3-ylethynyl)-8,9-dihydroimidazo[1′,2′:1,6]pyrido[2,3-d]pyrimidin-2-yl)amino)pyridin-2-yl)piperazine-1-carboxylate (15 mg, 18% yield) as a yellow solid. LCMS (M+H⁺) m/z: 580.3.

### Step 2: Synthesis of N-(2-methoxy-6-(piperazin-1-yl)pyridin-3-yl)-6-(pyridin-3-ylethynyl)-8,9-dihydroimidazo[1′,2′:1,6]pyrido[2,3-d]pyrimidin-2-amine

To a solution of tert-butyl 4-(6-methoxy-5-((6-(pyridin-3-ylethynyl)-8,9-dihydroimidazo[1′,2′:1,6]pyrido[2,3-d]pyrimidin-2-yl)amino)pyridin-2-yl)piperazine-1-carboxylate (15 mg, 0.04 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-methoxy-6-(piperazin-1-yl)pyridin-3-yl)-6-(pyridin-3-ylethynyl)-8,9-dihydroimidazo[1′,2′:1,6]pyrido[2,3-d]pyrimidin-2-amine (5 mg, 7% yield, TFA salt) as a yellow solid. ¹H NMR (400 MHz, CD₃OD): δ 8.90-8.83 (m, 2H), 8.63 (br, 1H), 8.42 (s, 1H), 8.29 (d, J=7.6 Hz, 1H), 8.12 (d, J=8.0 Hz, 1H), 7.56 (s, 1H), 6.50-6.48 (m, 1H), 4.73-4.70 (m, 2H), 4.27-4.20 (m, 2H), 3.98 (s, 3H), 3.89-3.82 (m, 4H), 3.35-3.31 (m, 4H). LCMS (M+H⁺) m/z: 480.1.

### Example 41: Preparation of N-(2-methoxy-6-(piperazin-1-yl)pyridin-3-yl)-6-(pyridin-4-yl)-8,9-dihydroimidazo[1′,2′:1,6]pyrido[2,3-d]pyrimidin-2-amine (Compound 41)

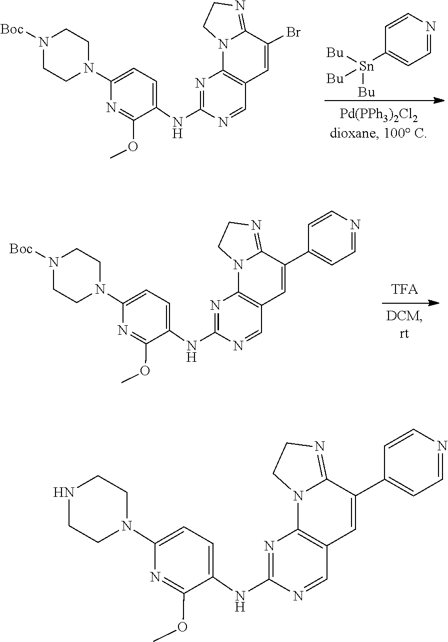

### Step 1: Synthesis of tert-butyl 4-(6-methoxy-5-((6-(pyridin-4-yl)-8,9-dihydroimidazo[1′,2′:1,6]pyrido[2,3-d]pyrimidin-2-yl)amino)pyridin-2-yl)piperazine-1-carboxylate

The mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (60 mg, 0.11 mmol), 4-(tributylstannyl) pyridine (119 mg, 0.32 mmol), Pd(PPh₃)₂Cl₂. (10.0 mg, 0.014 mmol) in dioxane (10 mL) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(6-methoxy-5-((6-(pyridin-4-yl)-8,9-dihydroimidazo[1′,2′:1,6]pyrido[2,3-d]pyrimidin-2-yl)amino)pyridin-2-yl)piperazine-1-carboxylate (20 mg, 42% yield) as a yellow solid. LCMS (M+H⁺) m/z: 556.2

### Step 2: Synthesis of N-(2-methoxy-6-(piperazin-1-yl)pyridin-3-yl)-6-(pyridin-4-yl)-8,9-dihydroimidazo[1′,2′:1,6]pyrido[2,3-d]pyrimidin-2-amine

To a solution of tert-butyl 4-(6-methoxy-5-((6-(pyridin-4-yl)-8,9-dihydroimidazo[1′,2′:1,6]pyrido[2,3-d]pyrimidin-2-yl)amino)pyridin-2-yl)piperazine-1-carboxylate (20 mg, 0.04 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give N-(2-methoxy-6-(piperazin-1-yl)pyridin-3-yl)-6-(pyridin-4-yl)-8,9-dihydroimidazo[1′,2′:1,6]pyrido[2,3-d]pyrimidin-2-amine (2 mg, 7% yield, TFA salt) as a yellow solid. ¹H NMR (400 MHz, CD₃OD): δ 8.97-8.95 (m, 3H), 8.42 (s, 1H), 8.31 (d, J=3.6 Hz, 1H), 7.94 (d, J=5.2 Hz, 2H), 6.51 (d, J=8.4 Hz, 1H), 4.74-4.69 (m, 2H), 4.23-4.18 (m, 2H), 3.98 (s, 3H), 3.84-3.81 (m, 4H), 3.36-3.31 (m, 4H). LCMS (M+H⁺) m/z: 456.1.

### Example 42: Preparation of 1-(4-(2-((2-methoxy-6-(piperazin-1-yl)pyridin-3-yl)amino)-8,9-dihydroimidazo[1′,2′:1,6]pyrido[2,3-d]pyrimidin-6-yl)phenyl)-3-methylpyrazin-2(1H)-one (Compound 42)

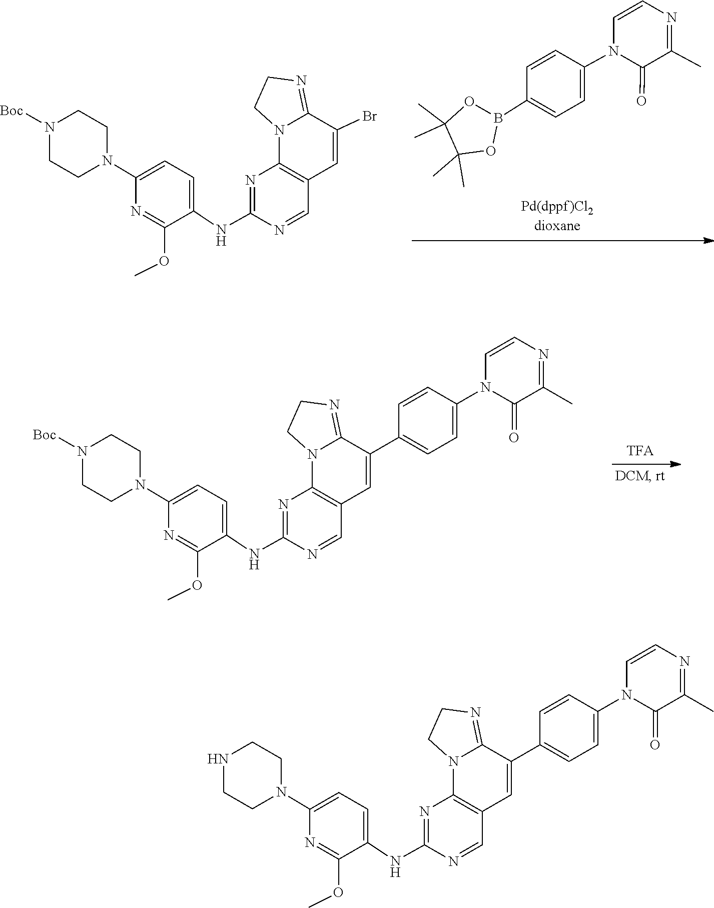

### Step 1: Synthesis of tert-butyl 4-(6-methoxy-5-((6-(4-(3-methyl-2-oxopyrazin-1(2H)-yl)phenyl)-8,9-dihydroimidazo[1′,2′:1,6]pyrido[2,3-d]pyrimidin-2-yl)amino)pyridin-2-yl)piperazine-1-carboxylate

A mixture of tert-butyl 4-(5-((6-bromo-8,9-dihydroimidazo[1′,2′:1,6]pyrido[2,3-d]pyrimidin-2-yl)amino)-6-methoxypyridin-2-yl)piperazine-1-carboxylate (70 mg, 0.13 mmol), 3-methyl-1-(4-(4,4,5,5-tetramethyl-1,3,2-dioxaborolan-2-yl)phenyl)pyrazin-2(1H)-one (78 mg, 0.26 mmol), Pd(dppf)Cl₂. DCM (18.0 mg, 0.02 mmol) and Na₂CO₃ (35 mg, 0.33 mmol) in dioxane/H₂O (10 mL, v/v=5/1) at room temperature was purged and degassed with N₂ for 3 times, and then stirred at 100° C. for 16 hours. The reaction mixture was concentrated to remove the solvent and the residue was purified by column chromatography on silica gel (DCM/CH₃OH=30:1) to afford tert-butyl 4-(6-methoxy-5-((6-(4-(3-methyl-2-oxopyrazin-1(2H)-yl)phenyl)-8,9-dihydroimidazo[1′,2′:1,6]pyrido[2,3-d]pyrimidin-2-yl)amino)pyridin-2-yl)piperazine-1-carboxylate (38 mg, 52% yield) as a yellow solid. LCMS (M+H⁺) m/z: 663.4.

### Step 2: Synthesis of 1-(4-(2-((2-methoxy-6-(piperazin-1-yl)pyridin-3-yl)amino)-8,9-dihydroimidazo[1′,2′:1,6]pyrido[2,3-d]pyrimidin-6-yl)phenyl)-3-methylpyrazin-2(1H)-one

To a solution of tert-butyl 4-(6-methoxy-5-((6-(4-(3-methyl-2-oxopyrazin-1(2H)-yl)phenyl)-8,9-dihydroimidazo[1′,2′:1,6]pyrido[2,3-d]pyrimidin-2-yl)amino)pyridin-2-yl)piperazine-1-carboxylate (38 mg, 0.06 mmol) in DCM (10 mL) was added TFA (2 mL). The mixture was stirred at room temperature for 2 hours under N₂. The residue was concentrated in vacuum and purified by prep-HPLC (0.1% TFA, CH₃CN in H₂O) to give 1-(4-(2-((2-methoxy-6-(piperazin-1-yl)pyridin-3-yl)amino)-8,9-dihydroimidazo[1′,2′:1,6]pyrido[2,3-d]pyrimidin-6-yl)phenyl)-3-methylpyrazin-2(1H)-one (18 mg, 57% yield, TFA salt) as a yellow solid. ¹H NMR (400 MHz, DMSO-d₆): δ 10.27 (br, 1H), 9.69 (s, 1H), 8.99-8.91 (m, 3H), 8.32 (s, 1H), 7.92-7.69 (m, 5H), 7.51 (d, J=4.8 Hz, 1H), 7.29 (d, J=4.4 Hz, 1H), 6.49 (d, J=8.4 Hz, 1H), 4.51-4.45 (m, 2H), 4.10-4.04 (m, 2H), 4.06 (s, 3H), 3.72-3.3.70 (m, 4H), 3.29-3.25 (m, 4H), 2.38 (s, 3H). LCMS (M+H⁺) m/z: 563.2.

### Example 43: Preparation of 6-phenyl-N-(pyridin-4-yl)-8,9-dihydroimidazo[1′,2′:1,6]pyrido[2,3-d]pyrimidin-2-amine (Compound 43)

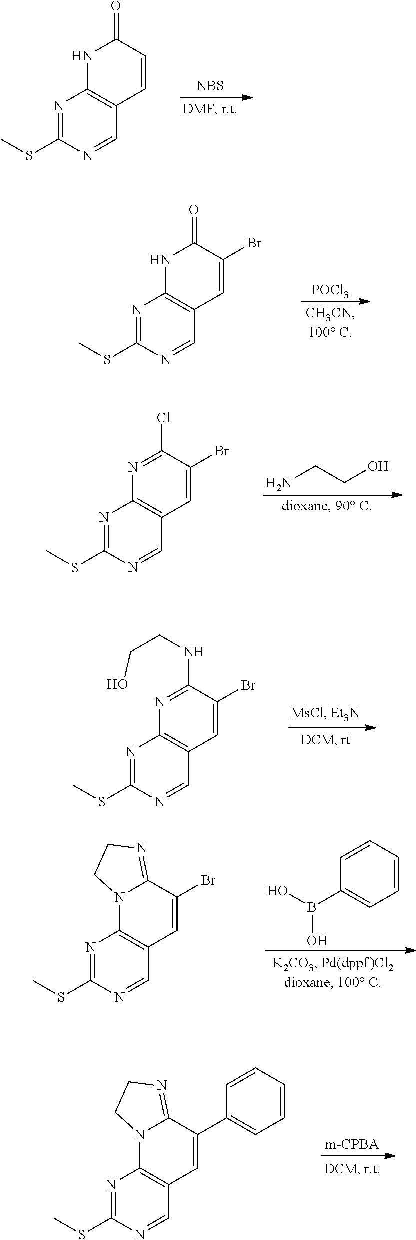

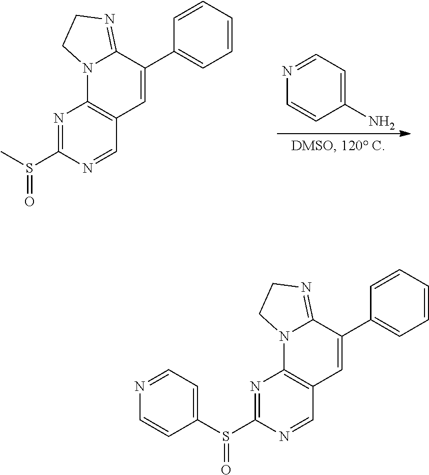 [SMILES: CS(=O)c1ncc2c(n1)N1CCN=C1C(c1ccccc1)=C2 ; Nc1ccncc1 ; O=S(c1ccncc1)c1ncc2c(n1)N1CCN=C1C(c1ccccc1)=C2]

### Step 1: Synthesis of 6-bromo-2-(methylthio)pyrido[2,3-d]pyrimidin-7(8H)-one

A mixture of 2-(methylthio)pyrido[2,3-d]pyrimidin-7(8H)-one (5.0 g, 24.84 mmol) and NBS (4.86 g, 27.32 mmol) was dissolved in DMF (200 mL). The mixture was stirred at room temperature for 5 hours. The mixture was added in H₂O (300 mL) and filtered to afford 6-bromo-2-(methylthio)pyrido[2,3-d]pyrimidin-7(8H)-one (3.0 g, 37% yield) as a white solid.

### Step 2: Synthesis of 6-Bromo-7-chloro-2-(methylthio)pyrido[2,3-d]pyrimidine

To a mixture of 6-bromo-2-(methylthio)pyrido[2,3-d]pyrimidin-7(8H)-one (2.5 g, 9.19 mmol) dissolved in CH₃CN (24 mL) was added POCl₃ (6 mL). The mixture was stirred at 100° C. for 16 hours. The mixture was concentrated to afford 6-bromo-7-chloro-2-(methylthio)pyrido[2,3-d]pyrimidine (3.0 g, crude) as a brown solid.

### Step 3: Synthesis of 2-((6-bromo-2-(methylthio)pyrido[2,3-d]pyrimidin-7-yl)amino)ethan-1-ol

A mixture of 6-bromo-7-chloro-2-(methylthio)pyrido[2,3-d]pyrimidine (3.0 g, 10.32 mmol) and 2-aminoethan-1-ol (3.15 g, 51.62 mmol) was dissolved in dioxane (30 mL). The mixture was stirred at 90° C. for 1 hour. The mixture was extracted by EtOAc (50 mL×2). The organic phase was washed with brine (50 mL), dried over Na₂SO₄ and concentrated in vacuum to afford 2-((6-bromo-2-(methylthio)pyrido[2,3-d]pyrimidin-7-yl)amino)ethan-1-ol (3.0 g, crude) as a yellow oil which was used next step directly.

### Step 4: Synthesis of 6-bromo-2-(methylthio)-8,9-dihydroimidazo[1′,2′:1,6]pyrido[2,3-d]pyrimidine

To a mixture of 2-((6-bromo-2-(methylthio)pyrido[2,3-d]pyrimidin-7-yl)amino)ethan-1-ol (3.0 g, 9.52 mmol) and Et₃N (4.82 g, 47.59 mmol) in DCM (30 mL) was added MsCl (3.27 g, 28.55 mmol). The mixture was stirred at room temperature for 16 hours. The mixture was added in H₂O (50 mL) and filtered to afford 6-bromo-2-(methylthio)-8,9-dihydroimidazo[1′,2′:1,6]pyrido[2,3-d]pyrimidine (2.0 g, crude) as a yellow solid.

### Step 5: Synthesis of 2-(methylthio)-6-phenyl-8,9-dihydroimidazo[1′,2′:1,6]pyrido[2,3-d]pyrimidine

A solution of 6-bromo-2-(methylthio)-8,9-dihydroimidazo[1′,2′:1,6]pyrido[2,3-d]pyrimidine (300 mg, 1.01 mmol), phenylboronic acid (182 mg, 1.51 mmol), Pd(dppf)Cl₂ (30 mg) and K₂CO₃ (419 mg, 3.03 mmol) in dioxane (30 mL) was stirred at 100° C. for 2 hours. The mixture was concentrated in vacuum to give crude which was purified on silica gel column flash chromatography (EtOAc/PE=1:1, v/v) to afford 2-(methylthio)-6-phenyl-8,9-dihydroimidazo[1′,2′:1,6]pyrido[2,3-d]pyrimidine (80 mg, 30% yield) as a yellow solid.

### Step 6: Synthesis of 2-(methylsulfinyl)-6-phenyl-8,9-dihydroimidazo[1′,2′:1,6]pyrido[2,3-d]pyrimidine

A solution of 2-(methylthio)-6-phenyl-8,9-dihydroimidazo[1′,2′:1,6]pyrido[2,3-d]pyrimidine (80 mg, 0.27 mmol) and m-CPBA (70 mg, 0.4 mmol) in DCM (10 mL) was stirred at room temperature for 1 hour. The reaction was concentrated to afford 2-(methylsulfinyl)-6-phenyl-8,9-dihydroimidazo[1′,2′:1,6]pyrido[2,3-d]pyrimidine (130 mg crude) which was used next step directly.

### Step 7: Synthesis of 6-phenyl-N-(pyridin-4-yl)-8,9-dihydroimidazo[1′,2′:1,6]pyrido[2,3-d]pyrimidin-2-amine

A mixture of 2-(methylsulfinyl)-6-phenyl-8,9-dihydroimidazo[1′,2′:1,6]pyrido[2,3-d]pyrimidine (130 mg, 0.43 mmol) and pyridin-4-amine (40 mg, 0.43 mmol) in DMSO (3 mL) was stirred at 120° C. for 1 hour. The mixture was purified by Prep-HPLC (0.1% NH₄HCO₃, CH₃CN in water) two times to afford 6-phenyl-N-(pyridin-4-yl)-8,9-dihydroimidazo[1′,2′:1,6]pyrido[2,3-d]pyrimidin-2-amine (26.6 mg, 36% yield) as a yellow solid. ¹H NMR (400 MHz, CD₃OD): δ 9.24 (d, J=8.0 Hz, 2H), 8.57 (s, 1H), 7.65-7.62 (m, 2H), 7.48 (s, 1H), 7.41-7.31 (m, 3H), 6.91 (d, J=8.0 Hz, 2H), 4.27-4.22 (m, 2H), 4.11-4.06 (m, 2H). LCMS (M+H⁺) m/z: 341.0.

### Example 44: Preparation of 6-(pyridin-2-yl)-N-(pyridin-4-yl)-8,9-dihydroimidazo[1′,2′:1,6]pyrido[2,3-d]pyrimidin-2-amine (Compound 44)

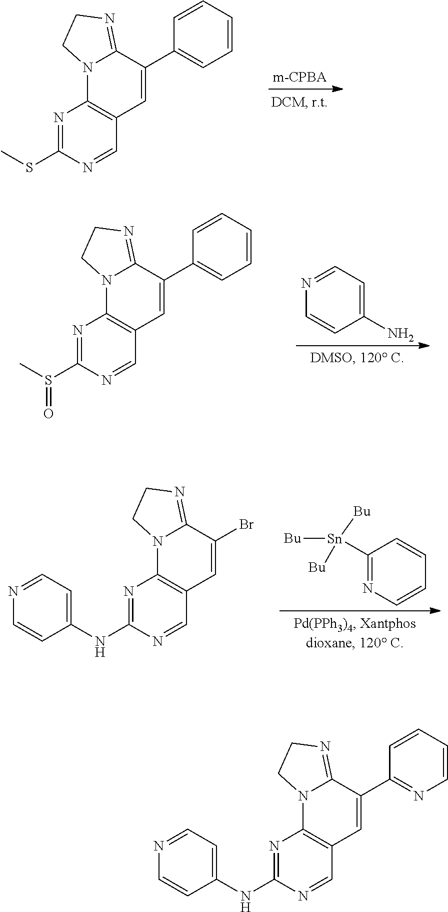

### Step 1: Synthesis of 6-bromo-2-(methylsulfinyl)-8,9-dihydroimidazo[1′,2′:1,6]pyrido[2,3-d]pyrimidine

A solution of 6-bromo-2-(methylthio)-8,9-dihydroimidazo[1′,2′:1,6]pyrido[2,3-d]pyrimidine (400 mg, 1.35 mmol) and m-CPBA (465 mg, 2.69 mmol) in DCM (30 mL) was stirred at room temperature for 10 minutes. The reaction was concentrated to afford 6-bromo-2-(methylsulfinyl)-8,9-dihydroimidazo[1′,2′:1,6]pyrido[2,3-d]pyrimidine (100 mg crude) which was used next step directly.

### Step 2: Synthesis of 6-bromo-N-(pyridin-4-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (600 mg, 1.92 mmol) and pyridin-4-amine (180 mg, 1.92 mmol) in DMSO (6 mL) was stirred at 120° C. for 1 hour. The mixture was purified by Prep-HPLC (0.1% Formic Acid, MeCN in water) to afford 6-bromo-N-(pyridin-4-yl)-8,9-dihydroimidazo[1′,2′:1,6]pyrido[2,3-d]pyrimidin-2-amine (100 mg, 15% yield) as a yellow solid.

### Step 3: Synthesis of 6-(pyridin-2-yl)-N-(pyridin-4-yl)-8,9-dihydroimidazo[1′,2′:1,6]pyrido[2,3-d]pyrimidin-2-amine

A solution of 6-bromo-N-(pyridin-4-yl)-8,9-dihydroimidazo[1′,2′:1,6]pyrido[2,3-d]pyrimidin-2-amine (45 mg, 0.13 mmol), 2-(tributylstannyl)pyridine (97 mg, 0.26 mmol), Pd(PPh₃)₄ (10 mg) and XantPhos (10 mg) in dioxane (3 mL) was stirred at 120° C. for 16 hours. The mixture was purified by Prep-HPLC (0.1% formic acid, CH₃CN in water) to afford 6-(pyridin-2-yl)-N-(pyridin-4-yl)-8,9-dihydroimidazo[1′,2′:1,6]pyrido[2,3-d]pyrimidin-2-amine (9.9 mg, 22% yield, formic acid salt) as a yellow solid. ¹H NMR (400 MHz, DMSO-d₆): δ 10.32 (s, 1H), 8.79 (d, J=8.0 Hz, 1H), 8.65-8.64 (m, 2H), 8.44-8.34 (m, 3H), 8.22 (s, 2H), 7.87-7.82 (m, 3H), 7.37-7.34 (m, 1H), 4.17-4.14 (m, 4H). LCMS (M+H⁺) m/z: 342.2.

### Example 45: Preparation of N,6-di(pyridin-4-yl)-8,9-dihydroimidazo[1′,2′:1,6]pyrido[2,3-d]pyrimidin-2-amine (Compound 45)

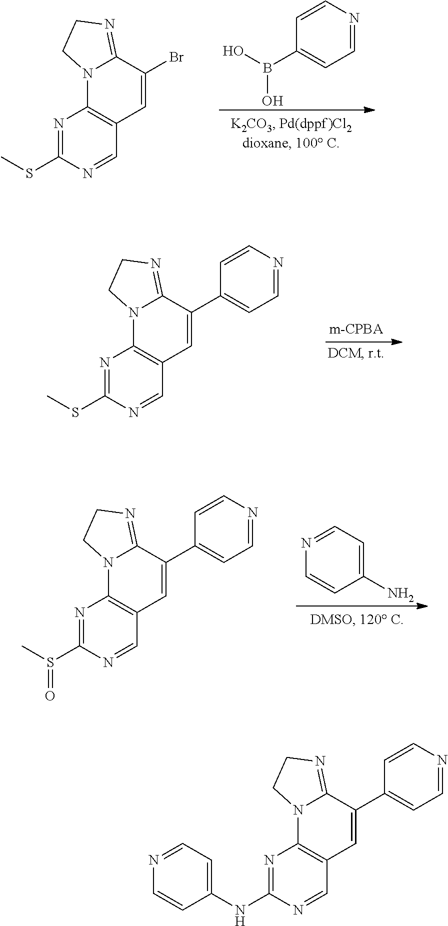 [SMILES: CSc1ncc2c(n1)N1CCN=C1C(Br)=C2 ; OB(O)c1ccncc1 ; CSc1ncc2c(n1)N1CCN=C1C(c1ccncc1)=C2 ; CS(=O)c1ncc2c(n1)N1CCN=C1C(c1ccncc1)=C2 ; Nc1ccncc1 ; C1=C(c2ccncc2)C2=NCCN2c2nc(Nc3ccncc3)ncc21]

### Step 1: Synthesis of 2-(methylthio)-6-(pyridin-4-yl)-8,9-dihydroimidazo[1′,2′:1,6]pyrido[2,3-d]pyrimidine

A solution of 6-bromo-2-(methylthio)-8,9-dihydroimidazo[1′,2′:1,6]pyrido[2,3-d]pyrimidine (300 mg, 1.01 mmol), pyridin-4-ylboronic acid (185 mg, 1.51 mmol), Pd(dppf)Cl₂ (30 mg) and K₂CO₃ (419 mg, 3.03 mmol) in dioxane (30 mL) was stirred at 100° C. for 2 hours. The mixture was concentrated in vacuum to give crude which was purified on silica gel column flash chromatography (EtOAc/PE=1:1, v/v) to afford 2-(methylthio)-6-(pyridin-4-yl)-8,9-dihydroimidazo[1′,2′:1,6]pyrido[2,3-d]pyrimidine (100 mg, 40% yield) as a yellow solid.

### Step 2: Synthesis of 2-(methylsulfinyl)-6-(pyridin-4-yl)-8,9-dihydroimidazo[1′,2′:1,6]pyrido[2,3-d]pyrimidine

A solution of 2-(methylthio)-6-(pyridin-4-yl)-8,9-dihydroimidazo[1′,2′:1,6]pyrido[2,3-d]pyrimidine (40 mg, 0.14 mmol) and m-CPBA (71 mg, 0.41 mmol) in DCM (10 mL) was stirred at room temperature for 10 minutes. The reaction was concentrated to afford 2-(methylsulfinyl)-6-(pyridin-4-yl)-8,9-dihydroimidazo[1′,2′:1,6]pyrido[2,3-d]pyrimidine (80 mg crude) which was used next step directly.

### Step 3: Synthesis of N,6-di(pyridin-4-yl)-8,9-dihydroimidazo[1′,2′:1,6]pyrido[2,3-d]pyrimidin-2-amine

A mixture of 2-(methylsulfinyl)-6-(pyridin-4-yl)-8,9-dihydroimidazo[1′,2′:1,6]pyrido[2,3-d]pyrimidine (80 mg, 0.26 mmol) and pyridin-4-amine (24 mg, 0.26 mmol) in DMSO (3 mL) was stirred at 120° C. for 1 hour. The mixture was purified by Prep-HPLC (0.1% formic acid, CH₃CN in water) to afford N,6-di(pyridin-4-yl)-8,9-dihydroimidazo[1′,2′:1,6]pyrido[2,3-d]pyrimidin-2-amine (12.2 mg, 14% yield) as a yellow solid. ¹H NMR (400 MHz, DMSO-d₆): δ 8.68-8.60 (m, 3H), 8.43 (d, J=8.0 Hz, 2H), 7.95-7.90 (m, 3H), 6.31 (d, J=8.0 Hz, 2H), 4.20-4.03 (m, 4H). LCMS (M+H⁺) m/z: 342.1.

### Example 46: Preparation of 6-(3-chloropyridin-4-yl)-N-(pyridin-4-yl)-8,9-dihydroimidazo[1′,2′:1,6]pyrido[2,3-d]pyrimidin-2-amine (Compound 46)

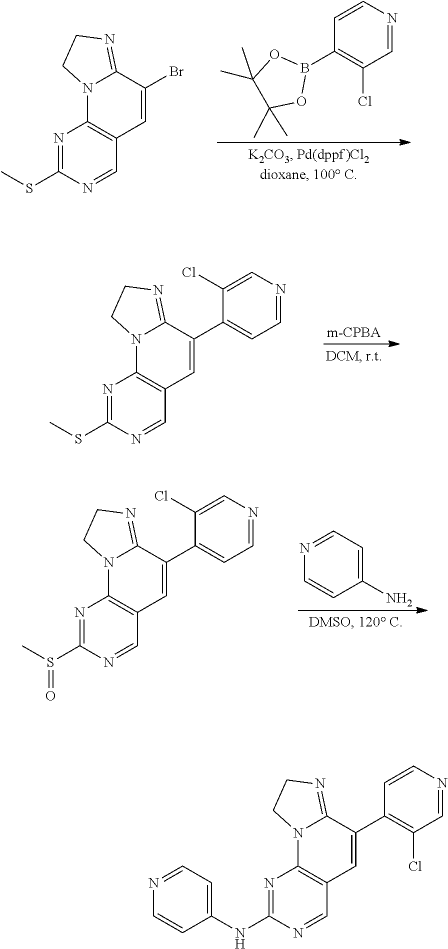

### Step 1: Synthesis of 6-(3-chloropyridin-4-yl)-2-(methylthio)-8,9-dihydroimidazo[1′,2′:1,6]pyrido[2,3-d]pyrimidine

A solution of 6-bromo-2-(methylthio)-8,9-dihydroimidazo[1′,2′:1,6]pyrido[2,3-d]pyrimidine (150 mg, 0.5 mmol), 3-chloro-4-(4,4,5,5-tetramethyl-1,3,2-dioxaborolan-2-yl)pyridine (181 mg, 0.76 mmol), Pd(dppf)Cl₂ (30 mg) and K₂CO₃ (209 mg, 1.51 mmol) in dioxane (30 mL) was stirred at 100° C. for 6 hours. The mixture was concentrated in vacuum to give crude which was purified on silica gel column flash chromatography (DCM/MeOH=20:1, v/v) to afford 6-(3-chloropyridin-4-yl)-2-(methylthio)-8,9-dihydroimidazo[1′,2′:1,6]pyrido[2,3-d]pyrimidine (80 mg, 48% yield) as a yellow solid.

### Step 2: Synthesis of 6-(3-chloropyridin-4-yl)-2-(methylsulfinyl)-8,9-dihydroimidazo[1′,2′:1,6]pyrido[2,3-d]pyrimidine

A solution of 6-(3-chloropyridin-4-yl)-2-(methylthio)-8,9-dihydroimidazo[1′,2′:1,6]pyrido[2,3-d]pyrimidine (70 mg, 0.21 mmol) and m-CPBA (55 mg, 0.32 mmol) in DCM (10 mL) was stirred at room temperature for 10 minutes. The reaction was concentrated to afford 6-(3-chloropyridin-4-yl)-2-(methylsulfinyl)-8,9-dihydroimidazo[1′,2′:1,6]pyrido[2,3-d]pyrimidine (120 mg crude) which was used next step directly.

### Step 3: Synthesis of 6-(3-chloropyridin-4-yl)-N-(pyridin-4-yl)-8,9-dihydroimidazo[1′,2′:1,6]pyrido[2,3-d]pyrimidin-2-amine

A mixture of 6-(3-chloropyridin-4-yl)-2-(methylsulfinyl)-8,9-dihydroimidazo[1′,2′:1,6]pyrido[2,3-d]pyrimidine (120 mg, 0.35 mmol) and pyridin-4-amine (33 mg, 0.35 mmol) in DMSO (3 mL) was stirred at 70° C. for 1 hour. The reaction was monitored by LCMS, showed worked. The mixture was purified by Prep-HPLC (0.1% formic acid, CH₃CN in water) to afford 6-(3-chloropyridin-4-yl)-N-(pyridin-4-yl)-8,9-dihydroimidazo[1′,2′:1,6]pyrido[2,3-d]pyrimidin-2-amine (33.8 mg, 26% yield, formic acid salt) as a yellow solid. ¹H NMR (400 MHz, DMSO-d₆): δ 9.58 (br, 1H), 9.19 (d, J=7.2 Hz, 2H), 8.78-8.74 (m, 2H), 8.62 (d, J=4.8 Hz, 1H), 8.50 (s, 1H), 7.66 (s, 1H), 7.56 (d, J=4.8 Hz, 1H), 7.08 (d, J=7.2 Hz, 2H), 4.21 (t, J=9.6 Hz, 2H), 4.06 (t, J=9.6 Hz, 2H). LCMS (M+H⁺) m/z: 376.2.

### Example 47: Preparation of 6-(2,4-dichlorophenyl)-N-(pyridin-4-yl)-8,9-dihydroimidazo[1′,2′:1,6]pyrido[2,3-d]pyrimidin-2-amine (Compound 47)

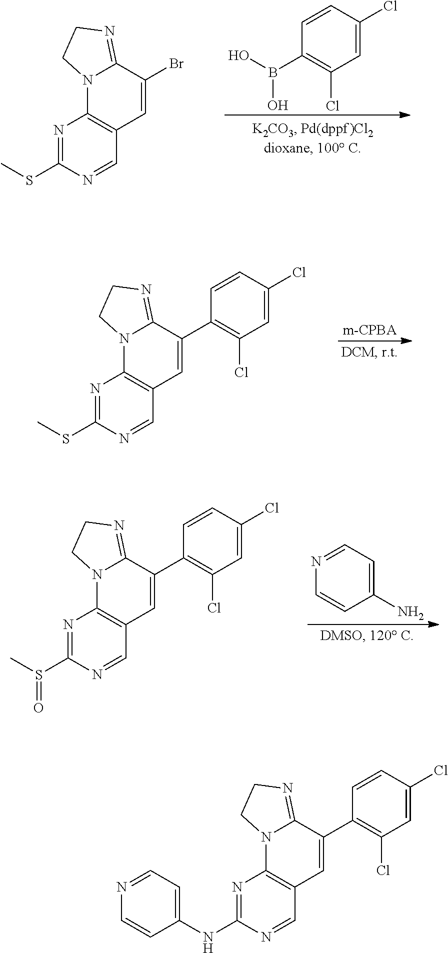 [SMILES: CSc1ncc2c(n1)N1CCN=C1C(Br)=C2 ; OB(O)c1ccc(Cl)cc1Cl ; CSc1ncc2c(n1)N1CCN=C1C(c1ccc(Cl)cc1Cl)=C2 ; CS(=O)c1ncc2c(n1)N1CCN=C1C(c1ccc(Cl)cc1Cl)=C2 ; Nc1ccncc1 ; Clc1ccc(C2=Cc3cnc(Nc4ccncc4)nc3N3CCN=C23)c(Cl)c1]

### Step 1: Synthesis of 6-(2,4-dichlorophenyl)-2-(methylthio)-8,9-dihydroimidazo[1′,2′:1,6]pyrido[2,3-d]pyrimidine

A solution of 6-bromo-2-(methylthio)-8,9-dihydroimidazo[1′,2′:1,6]pyrido[2,3-d]pyrimidine (300 mg, 1.01 mmol), (2,4-dichlorophenyl)boronic acid (288 mg, 1.51 mmol), Pd(dppf)Cl₂ (30 mg) and K₂CO₃ (419 mg, 3.03 mmol) in dioxane (30 mL) was stirred at 100° C. for 2 hours. The mixture was concentrated in vacuum to give crude which was purified on silica gel column flash chromatography (EtOAc/PE=1:1, v/v) to afford 6-(2,4-dichlorophenyl)-2-(methylthio)-8,9-dihydroimidazo[1′,2′:1,6]pyrido[2,3-d]pyrimidine (80 mg, 30% yield) as a yellow solid.

### Step 2: Synthesis of 6-(2,4-dichlorophenyl)-2-(methylsulfinyl)-8,9-dihydroimidazo[1′,2′:1,6]pyrido[2,3-d]pyrimidine

A solution of 6-(2,4-dichlorophenyl)-2-(methylthio)-8,9-dihydroimidazo[1′,2′:1,6]pyrido[2,3-d]pyrimidine (70 mg, 0.19 mmol) and m-CPBA (50 mg, 0.29 mmol) in DCM (10 mL) was stirred at room temperature for 1 hour. The reaction mixture was concentrated to afford 6-(2,4-dichlorophenyl)-2-(methylsulfinyl)-8,9-dihydroimidazo[1′,2′:1,6]pyrido[2,3-d]pyrimidine (100 mg crude) which was used next step directly.

### Step 3: Synthesis of 6-(2,4-dichlorophenyl)-N-(pyridin-4-yl)-8,9-dihydroimidazo[1′,2′:1,6]pyrido[2,3-d]pyrimidin-2-amine

A mixture of 6-(2,4-dichlorophenyl)-2-(methylsulfinyl)-8,9-dihydroimidazo[1′,2′:1,6]pyrido[2,3-d]pyrimidine (100 mg, 0.26 mmol) and pyridin-4-amine (25 mg, 0.26 mmol) in DMSO (3 mL) was stirred at 120° C. for 1 hour. The mixture was purified by Prep-HPLC (0.1% formic acid, CH₃CN in water) to afford 6-(2,4-dichlorophenyl)-N-(pyridin-4-yl)-8,9-dihydroimidazo[1′,2′:1,6]pyrido[2,3-d]pyrimidin-2-amine (38.5 mg, 36% yield, formic acid salt) as a yellow solid. ¹H NMR (400 MHz, CD₃OD): δ 9.25 (d, J=8.0 Hz, 2H), 8.59 (s, 1H), 8.45 (s, 1H), 7.52 (s, 1H), 7.42 (s, 1H), 7.35 (d, J=1.6 Hz, 2H), 6.92 (dd, J=6.0, 1.6 Hz, 2H), 4.27 (t, J=9.6 Hz, 2H), 4.04 (t, J=9.6 Hz, 2H). LCMS (M+H⁺) m/z: 409.0.

### Example 48: Preparation of 6-(2,6-dichlorophenyl)-N-(2-(4-methylpiperazin-1-yl)ethyl)-8,9-dihydroimidazo[1′,2′:1,6]pyrido[2,3-d]pyrimidin-2-amine (Compound 48)

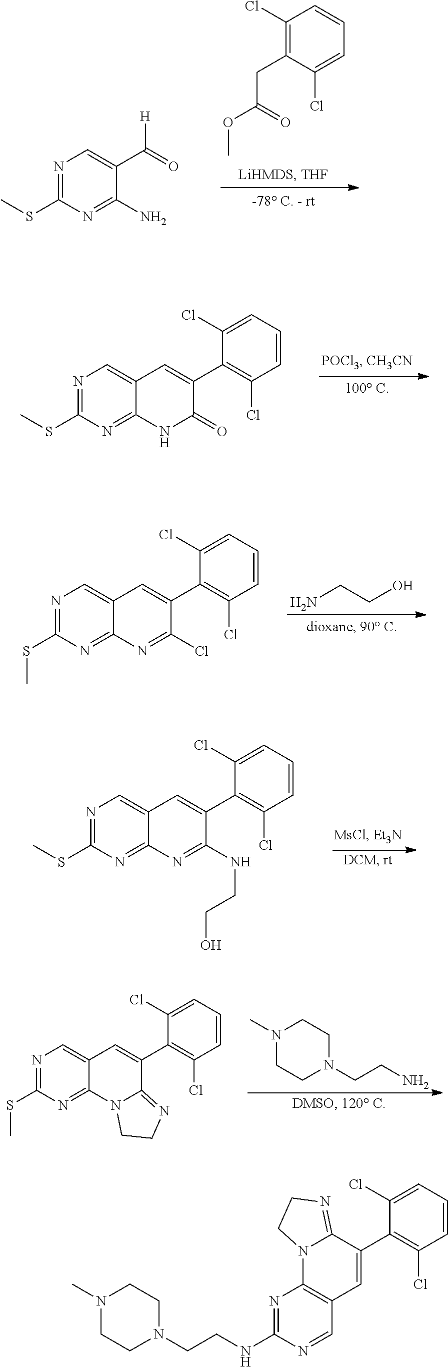

### Step 1: Synthesis of 6-(2,6-dichlorophenyl)-2-(methylthio)pyrido[2,3-d]pyrimidin-7(8H)-one

To a solution of methyl 2-(2,6-dichlorophenyl)acetate (4.38 g, 20 mmol) in THF (60 mL) was added dropwise LiHMDS (40 mL, 40 mmol) at −78° C. The mixture was stirred at −78° C. for 3 hours. Then 4-amino-2-(methylthio)pyrimidine-5-carbaldehyde (3.38 g, 20 mmol) was added. The mixture was stirred at room temperature for 16 hours. The reaction mixture was added NH₄Cl aq (30 mL). Extracted the mixture with EtOAc (50 mL×2) and the combined organic layers were washed with brine (30 mL), dried over anhydrous Na₂SO₄, filtered, concentrated in vacuum to give crude product which was purified on silica gel column flash chromatography (EtOAc/PE=1/1, v/v) to afford 6-(2,6-dichlorophenyl)-2-(methylthio)pyrido[2,3-d]pyrimidin-7(8H)-one (2.2 g, 32% yield) as a yellow solid. LCMS (M+H⁺) m/z: 337.9.

### Step 2: Synthesis of 7-chloro-6-(2,6-dichlorophenyl)-2-(methylthio)pyrido[2,3-d]pyrimidine

To a solution of 6-(2,6-dichlorophenyl)-2-(methylthio)pyrido[2,3-d]pyrimidin-7(8H)-one (2.2 g, 6.4 mmol) in CH₃CN (25 mL) was added POCl₃ (10 mL). The mixture was stirred at 100° C. for 16 hours. Concentrated the mixture to give the crude material. The crude material was extracted with EtOAc (20 mL×2) and the combined organic layers were washed with brine (20 mL), dried over anhydrous Na₂SO₄, filtered, concentrated in vacuum to give crude product which was purified on silica gel column flash chromatography (EtOAc/PE=1/4, v/v) to afford 7-chloro-6-(2,6-dichlorophenyl)-2-(methylthio)pyrido[2,3-d]pyrimidine (1.5 g, 65% yield) as a yellow solid. LCMS (M+H⁺) m/z: 355.8.

### Step 3: Synthesis of 2-((6-(2,6-dichlorophenyl)-2-(methylthio)pyrido[2,3-d]pyrimidin-7-yl)amino)ethan-1-ol

To a solution of 7-chloro-6-(2,6-dichlorophenyl)-2-(methylthio)pyrido[2,3-d]pyrimidine (712 mg, 2 mmol) in 1,4-dioxane (10 mL) was added 2-aminoethan-1-ol (366 mg, 6 mmol). The mixture was stirred at 90° C. for 16 hours. The mixture was extracted with EtOAc (10 mL×2) and the combined organic layers were washed with brine (10 mL), dried over anhydrous Na₂SO₄, filtered, concentrated in vacuum to give crude product which was purified on silica gel column flash chromatography (DCM/MeOH=10/1, v/v) to afford 2-((6-(2,6-dichlorophenyl)-2-(methylthio)pyrido[2,3-d]pyrimidin-7-yl)amino)ethan-1-ol (705 mg, 92% yield) as a yellow solid. LCMS (M+H⁺) m/z: 381.0.

### Step 4: Synthesis of 6-(2,6-dichlorophenyl)-2-(methylthio)-8,9-dihydroimidazo[1′,2′:1,6]pyrido[2,3-d]pyrimidine

To a solution of 2-((6-(2,6-dichlorophenyl)-2-(methylthio)pyrido[2,3-d]pyrimidin-7-yl)amino)ethan-1-ol (705 mg, 1.85 mmol) in DCM (30 mL) was added Et₃N (2.6 g, 25.9 mmol) and MsCl (1.47 g, 12.9 mmol). The mixture was stirred at room temperature for 2 hours. The mixture was extracted with DCM (10 mL×2) and the combined organic layers were washed with brine (10 mL), dried over anhydrous Na₂SO₄, filtered, concentrated in vacuum to give crude product which was purified on silica gel column flash chromatography (DCM/MeOH=20/1, v/v) to afford 6-(2,6-dichlorophenyl)-2-(methylthio)-8,9-dihydroimidazo[1′,2′:1,6]pyrido[2,3-d]pyrimidine (640 mg, 95% yield) as a yellow oil. LCMS (M+H⁺) m/z: 363.0.

### Step 5: Synthesis of 6-(2,6-dichlorophenyl)-N-(2-(4-methylpiperazin-1-yl)ethyl)-8,9-dihydroimidazo[1′,2′:1,6]pyrido[2,3-d]pyrimidin-2-amine

To a solution of 6-(2,6-dichlorophenyl)-2-(methylthio)-8,9-dihydroimidazo[1′,2′:1,6]pyrido[2,3-d]pyrimidine (50 mg, 0.14 mmol) in DMSO (3 mL) was added 2-(4-methylpiperazin-1-yl)ethan-1-amine (40 mg, 0.28 mmol). The mixture was stirred at 120° C. for 16 hours. The mixture was extracted with EtOAc (10 mL×2) and the combined organic layers were washed with brine (10 mL), dried over anhydrous Na₂SO₄, filtered, concentrated in vacuum to give crude product which was purified on prep-HPLC (10 mM NH₄HCO₃) to afford 6-(2,6-dichlorophenyl)-N-(2-(4-methylpiperazin-1-yl)ethyl)-8,9-dihydroimidazo[1′,2′:1,6]pyrido[2,3-d]pyrimidin-2-amine (5.6 mg, 9% yield) as a yellow solid. ¹H NMR (400 MHz, CD₃OD): δ 8.27 (s, 1H), 7.51-7.48 (m, 2H), 7.41-7.36 (m, 1H), 7.24 (s, 1H), 4.31-4.22 (m, 2H), 4.05-3.99 (m, 2H), 3.71-3.62 (m, 2H), 2.70-2.53 (m, 10H), 2.32 (s, 3H). LCMS (M+H⁺) m/z: 458.0.

### Example 49: Preparation of 6-(2,6-dichlorophenyl)-N-(2-(piperidin-4-yl)ethyl)-8,9-dihydroimidazo[1′,2′:1,6]pyrido[2,3-d]pyrimidin-2-amine (Compound 49)

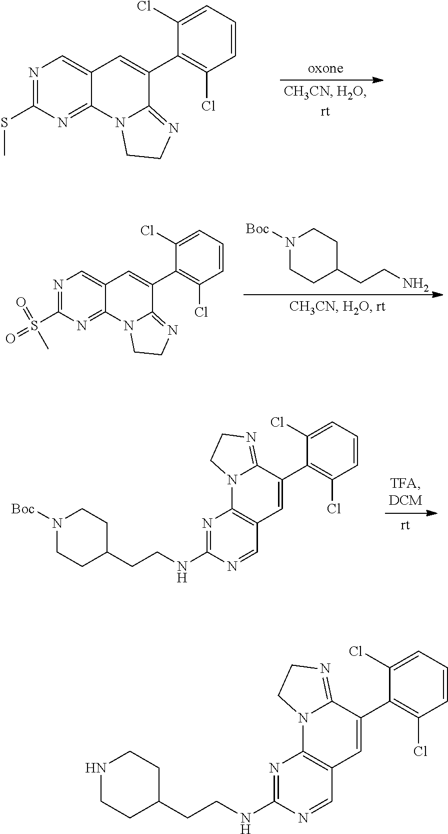 [SMILES: CSc1ncc2c(n1)N1CCN=C1C(c1c(Cl)cccc1Cl)=C2 ; CS(=O)(=O)c1ncc2c(n1)N1CCN=C1C(c1c(Cl)cccc1Cl)=C2 ; CC(C)(C)OC(=O)N1CCC(CCN)CC1 ; CC(C)(C)OC(=O)N1CCC(CCNc2ncc3c(n2)N2CCN=C2C(c2c(Cl)cccc2Cl)=C3)CC1 ; Clc1cccc(Cl)c1C1=Cc2cnc(NCCC3CCNCC3)nc2N2CCN=C12]

### Step 1: Synthesis of 6-(2,6-dichlorophenyl)-2-(methylsulfonyl)-8,9-dihydroimidazo[1′,2′:1,6]pyrido[2,3-d]pyrimidine

To a solution of 6-(2,6-dichlorophenyl)-2-(methylthio)-8,9-dihydroimidazo[1′,2′:1,6]pyrido[2,3-d]pyrimidine (200 mg, 0.82 mmol) in CH₃CN (3 mL) and H₂O (3 mL) was added oxone (509 mg, 0.83 mmol). The mixture was stirred at room temperature for 16 hours. The reaction mixture will be used directly in next reaction. LCMS (M+H⁺) m/z: 394.9.

### Step 2: Synthesis of tert-butyl 4-(2-((6-(2,6-dichlorophenyl)-8,9-dihydroimidazo[1′,2′:1,6]pyrido[2,3-d]pyrimidin-2-yl)amino)ethyl)piperidine-1-carboxylate

To a solution of 6-(2,6-dichlorophenyl)-2-(methylsulfonyl)-8,9-dihydroimidazo[1′,2′:1,6]pyrido[2,3-d]pyrimidine (200 mg, 0.5 mmol) in CH₃CN (3 mL) and H₂O (3 mL) was added tert-butyl 4-(2-aminoethyl)piperidine-1-carboxylate (342 mg, 1.5 mmol). The mixture was stirred at room temperature for 16 hours. The mixture was extracted with EtOAc (10 mL×2) and the combined organic layers were washed with brine (10 mL), dried over anhydrous Na₂SO₄, filtered, concentrated in vacuum to give crude product which was purified on silica gel column flash chromatography (DCM/MeOH=10/1, v/v) to afford tert-butyl 4-(2-((6-(2,6-dichlorophenyl)-8,9-dihydroimidazo[1′,2′:1,6]pyrido[2,3-d]pyrimidin-2-yl)amino)ethyl)piperidine-1-carboxylate (68 mg, 25% yield) as a yellow oil. LCMS (M+H⁺) m/z: 543.3.

### Step 3: Synthesis of 6-(2,6-dichlorophenyl)-N-(2-(piperidin-4-yl)ethyl)-8,9-dihydroimidazo[1′,2′:1,6]pyrido[2,3-d]pyrimidin-2-amine

To a solution of tert-butyl 4-(2-((6-(2,6-dichlorophenyl)-8,9-dihydroimidazo[1′,2′:1,6]pyrido[2,3-d]pyrimidin-2-yl)amino)ethyl)piperidine-1-carboxylate (68 mg, 0.13 mmol) in DCM (2 mL) was added TFA (1 mL). The mixture was stirred at room temperature for 1 hour. Concentrated the mixture to give the crude material. The crude material was purified by prep-HPLC (0.1% formic acid, CH₃CN in water) to afford 6-(2,6-dichlorophenyl)-N-(2-(piperidin-4-yl)ethyl)-8,9-dihydroimidazo[1′,2′:1,6]pyrido[2,3-d]pyrimidin-2-amine (11.5 mg, 20% yield, formic acid salt) as yellow oil. ¹H NMR (400 MHz, CD₃OD): δ 8.77-8.73 (m, 1H), 8.49 (s, 2H), 7.98-7.93 (m, 1H), 7.61 (d, J=8.0 Hz, 2H), 7.52 (t, J=8.0 Hz, 1H), 4.73-7.67 (m, 2H), 4.18-4.13 (m, 2H), 3.63 (t, J 7.2 Hz, 2H), 3.44-3.40 (m, 2H), 3.03-2.97 (m, 2H), 2.09-2.05 (m, 2H), 1.75-1.69 (m, 3H), 1.50-1.45 (m, 2H). LCMS (M+H⁺) m/z: 443.0.

### Example 50: Preparation of 6-(2,6-dichlorophenyl)-2-(piperazin-1-yl)-8,9-dihydroimidazo[1′,2′:1,6]pyrido[2,3-d]pyrimidine (Compound 50)

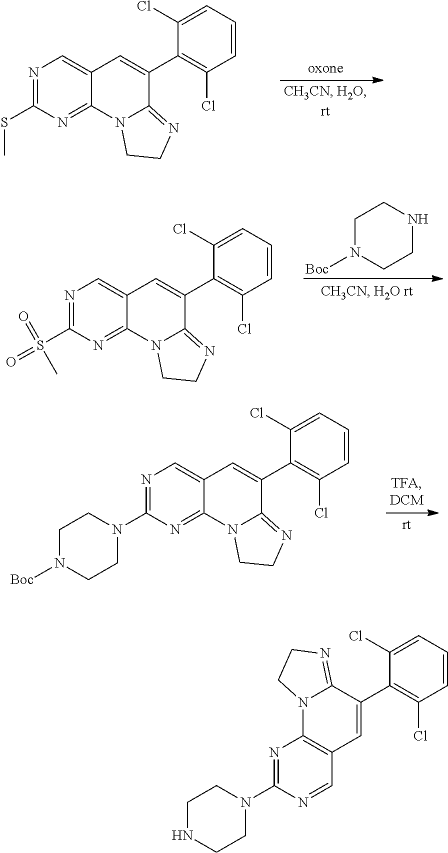 [SMILES: CSc1ncc2c(n1)N1CCN=C1C(c1c(Cl)cccc1Cl)=C2 ; CS(=O)(=O)c1ncc2c(n1)N1CCN=C1C(c1c(Cl)cccc1Cl)=C2 ; CC(C)(C)OC(=O)N1CCNCC1 ; CC(C)(C)OC(=O)N1CCN(c2ncc3c(n2)N2CCN=C2C(c2c(Cl)cccc2Cl)=C3)CC1 ; Clc1cccc(Cl)c1C1=Cc2cnc(N3CCNCC3)nc2N2CCN=C12]

### Step 1: Synthesis of 6-(2,6-dichlorophenyl)-2-(methylsulfonyl)-8,9-dihydroimidazo[1′,2′:1,6]pyrido[2,3-d]pyrimidine

To a solution of 6-(2,6-dichlorophenyl)-2-(methylthio)-8,9-dihydroimidazo[1′,2′:1,6]pyrido[2,3-d]pyrimidine (150 mg, 0.41 mmol) in CH₃CN (3 mL) and H₂O (3 mL) was added oxone (386 mg, 0.63 mmol). The mixture was stirred at room temperature for 16 hours. The reaction mixture will be used directly in next reaction. LCMS (M+H⁺) m/z: 394.9.

### Step 2: Synthesis of tert-butyl 4-(6-(2,6-dichlorophenyl)-8,9-dihydroimidazo[1′,2′:1,6]pyrido[2,3-d]pyrimidin-2-yl)piperazine-1-carboxylate

To the mixture of 6-(2,6-dichlorophenyl)-2-(methylsulfonyl)-8,9-dihydroimidazo[1′,2′:1,6]pyrido[2,3-d]pyrimidine (150 mg, 0.38 mmol) in CH₃CN (3 mL) and H₂O (3 mL) was added tert-butyl piperazine-1-carboxylate (212 mg, 1.14 mmol). The mixture was stirred at room temperature for 16 hours. The mixture was extracted with EtOAc (10 mL×2) and the combined organic layers were washed with brine (10 mL), dried over anhydrous Na₂SO₄, filtered, concentrated in vacuum to give crude product which was purified on silica gel column flash chromatography (DCM/MeOH=10/1, v/v) to afford tert-butyl 4-(6-(2,6-dichlorophenyl)-8,9-dihydroimidazo[1′,2′:1,6]pyrido[2,3-d]pyrimidin-2-yl)piperazine-1-carboxylate (60 mg, 31% yield) as a yellow oil. LCMS (M+H⁺) m/z: 501.0.

### Step 3: Synthesis of 6-(2,6-dichlorophenyl)-2-(piperazin-1-yl)-8,9-dihydroimidazo[1′,2′:1,6]pyrido[2,3-d]pyrimidine

To a solution of tert-butyl 4-(6-(2,6-dichlorophenyl)-8,9-dihydroimidazo[1′,2′:1,6]pyrido[2,3-d]pyrimidin-2-yl)piperazine-1-carboxylate (60 mg, 0.11 mmol) in DCM (1 mL) was added TFA (1 mL). The mixture was stirred at room temperature for 1 hour. Concentrated the mixture to give the crude material. The crude material was purified by prep-HPLC (0.1% formic acid, CH₃CN in water) to afford 6-(2,6-dichlorophenyl)-2-(piperazin-1-yl)-8,9-dihydroimidazo[1′,2′:1,6]pyrido[2,3-d]pyrimidine (9.4 mg, 17% yield) as yellow solid. ¹H NMR (400 MHz, CD₃OD): δ 8.85 (s, 1H), 8.02 (s, 1H), 7.58-7.55 (m, 2H), 7.50-7.45 (m, 1H), 4.70-4.65 (m, 2H), 4.25-4.22 (m, 4H), 4.15-4.11 (m, 2H), 3.30-3.25 (m, 4H). LCMS (M+H⁺) m/z: 401.0.

### Example 51: Preparation of 6-(2,6-dichlorophenyl)-2-(4-(pyridin-4-yl)piperazin-1-yl)-8,9-dihydroimidazo[1′,2′:1,6]pyrido[2,3-d]pyrimidine (Compound 51)

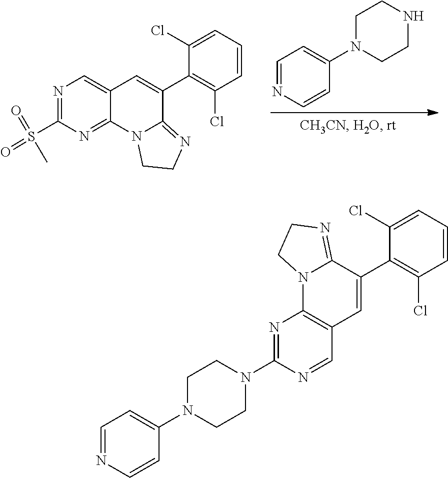

### Step 1: Synthesis of 6-(2,6-dichlorophenyl)-2-(4-(pyridin-4-yl)piperazin-1-yl)-8,9-dihydroimidazo[1′,2′:1,6]pyrido[2,3-d]pyrimidine

To a solution of 6-(2,6-dichlorophenyl)-2-(methylsulfonyl)-8,9-dihydroimidazo[1′,2′:1,6]pyrido[2,3-d]pyrimidine (300 mg, 0.76 mmol) in CH₃CN (3 mL) and H₂O (3 mL) was added 1-(pyridin-4-yl)piperazine (371 mg, 2.28 mmol). The mixture was stirred at room temperature for 16 hours. The crude material was purified by prep-HPLC (10 mM NH₄HCO₃) to afford 6-(2,6-dichlorophenyl)-2-(4-(pyridin-4-yl)piperazin-1-yl)-8,9-dihydroimidazo[1′,2′:1,6]pyrido[2,3-d]pyrimidine (19.0 mg, 5% yield) as yellow solid. ¹H NMR (400 MHz, DMSO-d₆): δ 9.16 (d, J=7.6 Hz, 2H), 8.74 (s, 1H), 7.62-7.56 (m, 3H), 7.52-7.47 (m, 1H), 7.40 (d, J=8.0 Hz, 2H), 4.26-4.23 (m, 2H), 4.06-4.03 (m, 2H), 3.81-3.72 (m, 4H), 3.31-3.28 (m, 2H), 2.91-2.82 (m, 2H). LCMS (M+H⁺) m/z: 478.0.

### Example 52: Preparation of 6-(2,6-dichlorophenyl)-N-(pyridin-4-yl)-8,9-dihydroimidazo[1′,2′:1,6]pyrido[2,3-d]pyrimidin-2-amine (Compound 52)

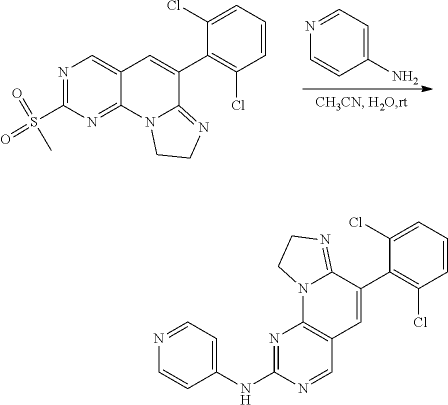

### Step 1: Synthesis of 6-(2,6-dichlorophenyl)-N-(pyridin-4-yl)-8,9-dihydroimidazo[1′,2′:1,6]pyrido[2,3-d]pyrimidin-2-amine

To a solution of 6-(2,6-dichlorophenyl)-2-(methylsulfonyl)-8,9-dihydroimidazo[1′,2′:1,6]pyrido[2,3-d]pyrimidine (300 mg, 0.76 mmol) in CH₃CN (3 mL) and H₂O (3 mL) was added pyridin-4-amine (214 mg, 2.28 mmol). The mixture was stirred at room temperature for 16 hours. Concentrated the mixture to give the crude material. The crude material was purified by prep-HPLC (10 mM NH₄HCO₃) to afford 6-(2,6-dichlorophenyl)-N-(pyridin-4-yl)-8,9-dihydroimidazo[1′,2′:1,6]pyrido[2,3-d]pyrimidin-2-amine (10.2 mg, 3% yield) as yellow solid. ¹H NMR (400 MHz, DMSO-d₆): δ 9.01-9.96 (m, 3H), 8.69 (s, 1H), 7.61 (d, J=8.0 Hz, 2H), 7.51 (s, 1H), 7.50-7.47 (m, 1H), 6.86-6.84 (m, 2H), 4.22 (t, J=9.2 Hz, 2H), 4.02 (t, J 9.2 Hz, 2H). LCMS (M+H⁺) m/z: 409.0.

### Example 53: Preparation of 6-phenyl-N-(pyridin-4-yl)-9,10-dihydro-8H-pyrido[1,6-a:2,3-d′]dipyrimidin-2-amine (Compound 53)

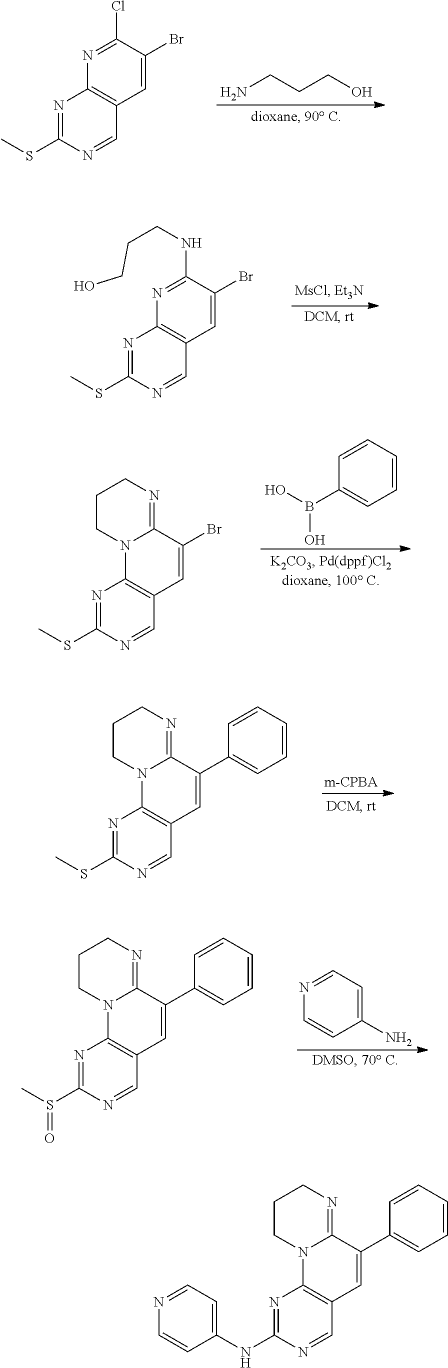

### Step 1: Synthesis of 3-((6-bromo-2-(methylthio)pyrido[2,3-d]pyrimidin-7-yl)amino)propan-1-ol

A mixture of 6-bromo-7-chloro-2-(methylthio)pyrido[2,3-d]pyrimidine (6.5 g, 22.37 mmol) and 3-aminopropan-1-ol (8.4 g, 111.85 mmol) was dissolved in dioxane (30 ml). The mixture was stirred at 90° C. for 1 hour. The mixture was extracted by EtOAc (50 mL×2). The organic phase was washed with brine (50 mL), dried over Na₂SO₄ and concentrated in vacuum to give crude which was purified on silica gel column flash chromatography (EtOAc/PE=3:1, v/v) to afford 3-((6-bromo-2-(methylthio)pyrido[2,3-d]pyrimidin-7-yl)amino)propan-1-ol (5.0 g, 68% yield) as a yellow solid.

### Step 2: Synthesis of 6-bromo-2-(methylthio)-9,10-dihydro-8H-pyrido[1,6-a:2,3-d′]dipyrimidine

A mixture of 3-((6-bromo-2-(methylthio)pyrido[2,3-d]pyrimidin-7-yl)amino)propan-1-ol (2.0 g, 6.07 mmol) and Et₃N (3 mL) was dissolved in DCM (30 mL), and then added MsCl (1 mL). The mixture was stirred at room temperature for 5 hours. The mixture was concentrated in vacuum to give crude which was purified on silica gel column flash chromatography (EtOAc/PE=3:1, v/v) to afford 6-bromo-2-(methylthio)-9,10-dihydro-8H-pyrido[1,6-a:2,3-d′]dipyrimidine (1.2 g, 63% yield) as a yellow solid.

### Step 3: Synthesis of 2-(methylthio)-6-phenyl-9,10-dihydro-8H-pyrido[1,6-a:2,3-d′]dipyrimidine

A solution of 6-bromo-2-(methylthio)-9,10-dihydro-8H-pyrido[1,6-a:2,3-d′]dipyrimidine (200 mg, 0.64 mmol), phenylboronic acid (117 mg, 0.96 mmol), Pd(dppf)Cl₂ (30 mg) and K₂CO₃ (266 mg, 1.92 mmol) in dioxane (30 mL) was stirred at 100° C. for 2 hours. The mixture was concentrated in vacuum to give crude which was purified on silica gel column flash chromatography (MeOH/DCM=10:1, v/v) to afford 2-(methylthio)-6-phenyl-9,10-dihydro-8H-pyrido[1,6-a:2,3-d′]dipyrimidine (150 mg, 70% yield) as a yellow solid.

### Step 4: Synthesis of 2-(methylsulfinyl)-6-phenyl-9,10-dihydro-8H-pyrido[1,6-a:2,3-d′]dipyrimidine

A solution of 2-(methylthio)-6-phenyl-9,10-dihydro-8H-pyrido[1,6-a:2,3-d′]dipyrimidine (50 mg, 0.16 mmol) and m-CPBA (112 mg, 0.62 mmol) in DCM (15 mL) was stirred at room temperature for 10 minutes. The reaction was concentrated to afford 2-(methylsulfinyl)-6-phenyl-9,10-dihydro-8H-pyrido[1,6-a:2,3-d′]dipyrimidine (150 mg, crude) which was used in next step directly.

### Step 5: Synthesis of 6-phenyl-N-(pyridin-4-yl)-9,10-dihydro-8H-pyrido[1,6-a:2,3-d′]dipyrimidin-2-amine

A mixture of 2-(methylsulfinyl)-6-phenyl-9,10-dihydro-8H-pyrido[1,6-a:2,3-d′]dipyrimidine (150 mg, 0.46 mmol) and pyridin-4-amine (44 mg, 0.46 mmol) in DMSO (3 mL) was stirred at 70° C. for 1 hour. The mixture was purified by Prep-HPLC (0.1% NH₄HCO₃) and (0.1% formic acid, CH₃CN in water) to afford 6-phenyl-N-(pyridin-4-yl)-9,10-dihydro-8H-pyrido[1,6-a:2,3-d′]dipyrimidin-2-amine (5.4 mg, 0.03% yield) as a yellow solid. ¹H NMR (400 MHz, DMSO-d₆): δ 8.84-8.82 (m, 2H), 8.66 (s, 1H), 7.52 (d, J=6.4 Hz, 2H), 7.43-7.31 (m, 5H), 6.66-6.61 (s, 1H), 4.23 (s, 2H), 3.54 (s, 2H), 1.92 (s, 2H). LCMS (M+H⁺) m/z: 354.6.

### Example 54: Preparation of 6-(2-chlorophenyl)-N-(pyridin-4-yl)-9,10-dihydro-8H-pyrido[1,6-a:2,3-d′]dipyrimidin-2-amine (Compound 54)

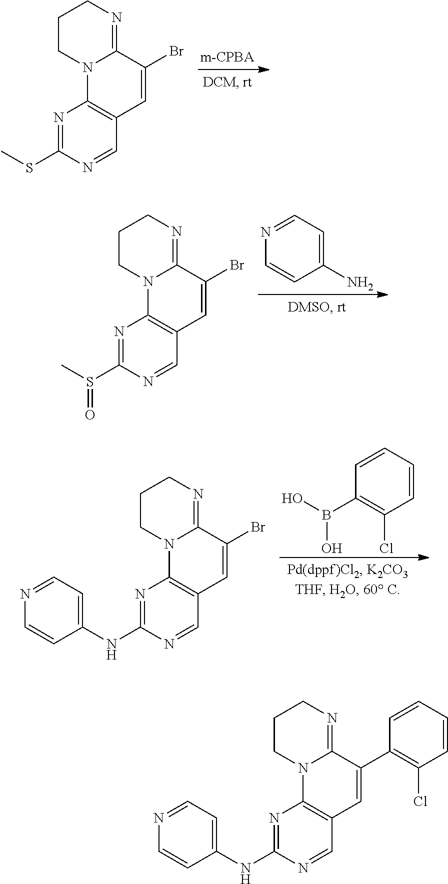

### Step 1: Synthesis of 6-bromo-2-(methylsulfinyl)-9,10-dihydro-8H-pyrido[1,6-a:2,3-d′]dipyrimidine

To a mixture of 6-bromo-2-(methylthio)-9,10-dihydro-8H-pyrido[1,6-a:2,3-d′]dipyrimidine (200 mg, 0.64 mmol) in DCM (5 mL) was added m-CPBA (332 mg, 1.93 mmol) at room temperature. The mixture was stirred at room temperature for 1 hour. The reaction mixture was concentrated to remove DCM to give crude 6-bromo-2-(methylsulfinyl)-9,10-dihydro-8H-pyrido[1,6-a:2,3-d′]dipyrimidine (241 mg) which was used to next step directly. LCMS (M+H⁺) m/z: 326.8.

### Step 2: Synthesis of 6-bromo-N-(pyridin-4-yl)-9,10-dihydro-8H-pyrido[1,6-a:2,3-d′]dipyrimidin-2-amine

To a solution of 6-bromo-2-(methylsulfinyl)-9,10-dihydro-8H-pyrido[1,6-a:2,3-d′]dipyrimidine (241 mg, 0.74 mmol) in DMSO (4 mL) was added pyridin-4-amine (83 mg, 0.88 mmol). The mixture was stirred at room temperature for 1 hour. The mixture was purified by Prep-HPLC (0.1% formic acid, CH₃CN in water) to afford 6-bromo-N-(pyridin-4-yl)-9,10-dihydro-8H-pyrido[1,6-a:2,3-d′]dipyrimidin-2-amine (60 mg, 23% yield) as a yellow solid. LCMS (M+H⁺) m/z: 356.5.

### Step 3: Synthesis of 6-(2-chlorophenyl)-N-(pyridin-4-yl)-9,10-dihydro-8H-pyrido[1,6-a:2,3-d′]dipyrimidin-2-amine

To a solution of 6-bromo-N-(pyridin-4-yl)-9,10-dihydro-8H-pyrido[1,6-a:2,3-d′]dipyrimidin-2-amine (20 mg, 0.06 mmol) in THF (2 mL) was added (2-chlorophenyl)boronic acid (12 mg, 0.072 mmol), Pd(dppf)Cl₂ (2 mg, 0.003 mmol), K₂CO₃ (26 mg, 0.18 mmol) and H₂O (0.2 mL). The mixture was stirred at 60° C. for 2 hours. Concentrated the mixture to give the crude product which was purified by prep-HPLC (0.1% formic acid, CH₃CN in water) to afford 6-(2-chlorophenyl)-N-(pyridin-4-yl)-9,10-dihydro-8H-pyrido[1,6-a:2,3-d′]dipyrimidin-2-amine (6.2 mg, 29% yield, formic acid salt) as a yellow solid. ¹H NMR (400 MHz, DMSO-d₆): δ 9.27 (d, J=7.6 Hz, 2H), 9.22 (s, 1H), 8.74 (s, 1H), 8.39 (s, 2H), 7.51-7.48 (m, 1H), 7.42-7.33 (m, 4H), 7.05 (d, J=8.0 Hz, 2H), 4.27-4.24 (m, 2H), 3.49-3.46 (m, 2H), 1.92-1.89 (m, 2H). LCMS (M+H⁺) m/z: 389.0.

### Example 55: Preparation of N-(3-fluoropyridin-4-yl)-6-phenyl-9,10-dihydro-8H-pyrido[1,6-a:2,3-d′]dipyrimidin-2-amine (Compound 55)

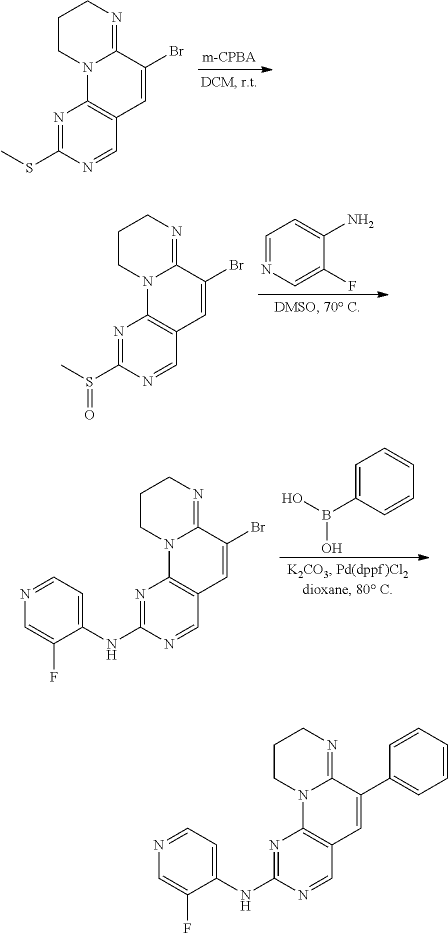

### Step 1: Synthesis of 6-bromo-2-(methylsulfinyl)-9,10-dihydro-8H-pyrido[1,6-a:2,3-d′]dipyrimidine

A solution of 6-bromo-2-(methylthio)-9,10-dihydro-8H-pyrido[1,6-a:2,3-d′]dipyrimidine (150 mg, 0.48 mmol) and m-CPBA (169 mg, 0.96 mmol) in DCM (80 mL) was stirred at room temperature for 10 minutes. The reaction was concentrated to afford 6-bromo-2-(methylsulfinyl)-9,10-dihydro-8H-pyrido[1,6-a:2,3-d′]dipyrimidine (400 mg, crude) which was used in the next step directly.

### Step 2: Synthesis of 6-bromo-N-(3-fluoropyridin-4-yl)-9,10-dihydro-8H-pyrido[1,6-a:2,3-d′]dipyrimidin-2-amine

A mixture of 6-bromo-2-(methylsulfinyl)-9,10-dihydro-8H-pyrido[1,6-a:2,3-d′]dipyrimidine (400 mg, 1.22 mmol) and 3-fluoropyridin-4-amine (137 mg, 1.22 mmol) in DMSO (5 mL) was stirred at 70° C. for 5 hours. The mixture was purified by Prep-HPLC (0.1% NH₄HCO₃) to afford 6-bromo-N-(3-fluoropyridin-4-yl)-9,10-dihydro-8H-pyrido[1,6-a:2,3-d′]dipyrimidin-2-amine (41 mg, 23% yield for two steps) as a yellow solid.

### Step 3: Synthesis of N-(3-Fluoropyridin-4-yl)-6-phenyl-9,10-dihydro-8H-pyrido[1,6-a:2,3-d′]dipyrimidin-2-amine

A solution of 6-bromo-N-(3-fluoropyridin-4-yl)-9,10-dihydro-8H-pyrido[1,6-a:2,3-d′]dipyrimidin-2-amine (36 mg, 0.09 mmol), phenylboronic acid (17 mg, 0.14 mmol), Pd(dppf)Cl₂ (5 mg) and K₂CO₃ (39 mg, 0.28 mmol) in dioxane (20 mL) was stirred at 70° C. for 2 hours. The mixture was purified by Prep-HPLC (0.1% NH₄HCO₃) twice to afford N-(3-fluoropyridin-4-yl)-6-phenyl-9,10-dihydro-8H-pyrido[1,6-a:2,3-d′]dipyrimidin-2-amine (6.4 mg, 18% yield) as a yellow solid. ¹H NMR (400 MHz, DMSO-d₆): δ 8.63 (d, J=8.4 Hz, 1H), 8.59 (s, 1H), 8.46 (dd, J=8.0, 1.2 Hz, 1H), 7.53-7.50 (m, 2H), 7.42-7.29 (m, 5H), 6.45 (t, J=8.4 Hz, 1H), 4.22 (t, J=5.6 Hz, 2H), 3.52 (t, J=5.6 Hz, 2H), 1.95-1.89 (m, 2H). LCMS (M+H⁺) m/z: 373.1.

### Example 56: Preparation of N-(2-fluorophenyl)-6-phenyl-9,10-dihydro-8H-pyrido[1,6-a:2,3-d′]dipyrimidin-2-amine (Compound 56)

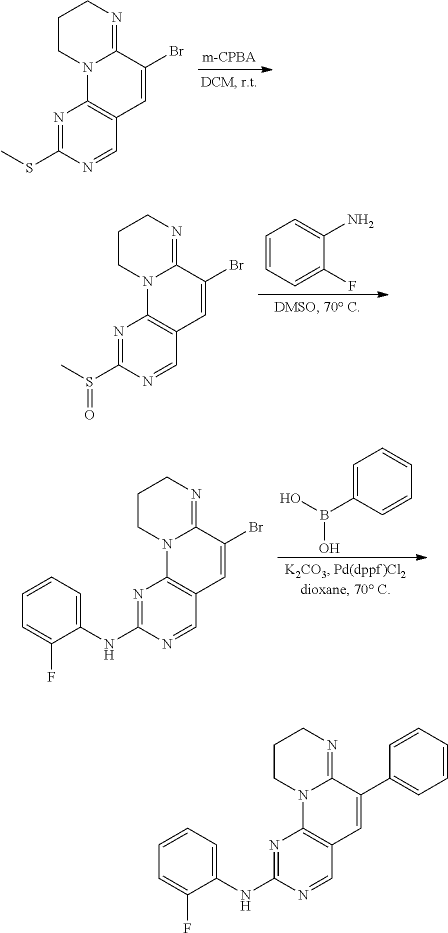

### Step 1: Synthesis of 6-bromo-2-(methylsulfinyl)-9,10-dihydro-8H-pyrido[1,6-a:2,3-d′]dipyrimidine

A solution of 6-bromo-2-(methylthio)-9,10-dihydro-8H-pyrido[1,6-a:2,3-d′]dipyrimidine (300 mg, 0.96 mmol) and m-CPBA (333 mg, 1.93 mmol) in DCM (100 mL) was stirred at room temperature for 10 minutes. The reaction was concentrated to afford 6-bromo-2-(methylsulfinyl)-9,10-dihydro-8H-pyrido[1,6-a:2,3-d′]dipyrimidine (500 mg, crude) which was used in the next step directly.

### Step 2: Synthesis of 6-bromo-N-(2-fluorophenyl)-9,10-dihydro-8H-pyrido[1,6-a:2,3-d′]dipyrimidin-2-amine

A mixture of 6-bromo-2-(methylsulfinyl)-9,10-dihydro-8H-pyrido[1,6-a:2,3-d′]dipyrimidine (500 mg, 1.53 mmol) and 2-fluoroaniline (849 mg, 7.64 mmol) in DMSO (5 mL) was stirred at 70° C. for 8 hours. The mixture was purified by Prep-HPLC (0.1% formic acid, CH₃CN in water) to afford 6-bromo-N-(2-fluorophenyl)-9,10-dihydro-8H-pyrido[1,6-a:2,3-d′]dipyrimidin-2-amine (80 mg, 22% yield for 2 steps) as a yellow solid.

### Step 3: Synthesis of N-(2-fluorophenyl)-6-phenyl-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (30 mg, 0.08 mmol), phenylboronic acid (15 mg, 0.12 mmol), Pd(dppf)Cl₂ (5 mg) and K₂CO₃ (33 mg, 0.24 mmol) in dioxane (2 mL) was stirred at 70° C. for 2 hours. The mixture was purified by Prep-HPLC (0.1% formic acid, CH₃CN in water) and (0.1% NH₄HCO₃) to afford N-(2-fluorophenyl)-6-phenyl-9,10-dihydro-8H-pyrido[1,6-a:2,3-d′]dipyrimidin-2-amine (8.4 mg, 28% yield) as a yellow solid. ¹H NMR (400 MHz, DMSO-d₆): δ 9.22 (s, 1H), 8.35 (s, 1H), 7.84-7.80 (m, 1H), 7.48 (dd, J=8.0, 1.6 Hz, 2H), 7.36-7.13 (m, 7H), 4.04 (t, J=5.6 Hz, 2H), 3.45 (t, J=5.6 Hz, 2H), 1.88-1.82 (m, 2H). LCMS (M+H⁺) m/z: 372.1.

### Example 57: Preparation of N-(2-fluorophenyl)-6-(1H-indol-4-yl)-9,10-dihydro-8H-pyrido[1,6-a:2,3-d′]dipyrimidin-2-amine (Compound 57)

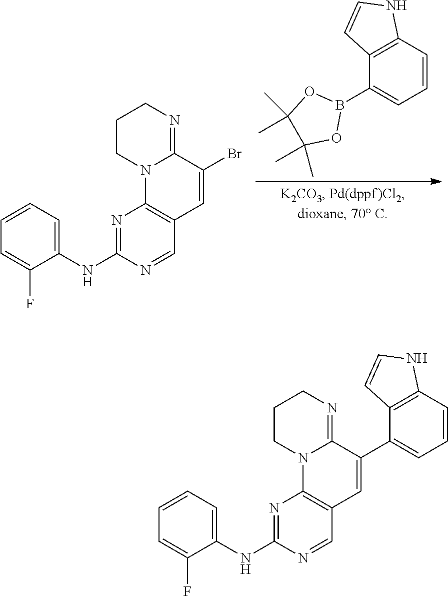

### Step 1: Synthesis of N-(2-fluorophenyl)-6-(1H-indol-4-yl)-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (30 mg, 0.08 mmol), 4-(4,4,5,5-tetramethyl-1,3,2-dioxaborolan-2-yl)-1H-indole (29 mg, 0.12 mmol), Pd(dppf)Cl₂ (5 mg) and K₂CO₃ (33 mg, 0.24 mmol) in dioxane (2 mL) was stirred at 70° C. for 2 hours. The mixture was purified by Prep-HPLC (0.1% formic acid, CH₃CN in water) to afford N-(2-fluorophenyl)-6-(1H-indol-4-yl)-9,10-dihydro-8H-pyrido[1,6-a:2,3-d′]dipyrimidin-2-amine (13 mg, 28% yield, formic acid salt) as a yellow solid. ¹H NMR (400 MHz, DMSO-d₆): δ 11.16 (s, 1H), 9.46 (s, 1H), 8.53 (s, 1H), 8.29 (s, 1H), 7.83 (t, J=5.6 Hz, 1H), 7.45-7.37 (m, 2H), 7.34-7.32 (m, 1H), 7.31-7.23 (m, 1H), 7.23-7.15 (m, 2H), 7.14-7.08 (m, 1H), 7.02 (dd, J=7.2, 0.8 Hz, 1H), 6.32-6.30 (m, 1H), 4.16 (t, J=5.6 Hz, 2H), 3.37 (t, J=5.6 Hz, 2H), 1.95-1.93 (m, 2H). LCMS (M+H⁺) m/z: 411.1.

### Example 58: Preparation of N-(2-fluorophenyl)-6-(1H-indazol-4-yl)-9,10-dihydro-8H-pyrido[1,6-a:2,3-d′]dipyrimidin-2-amine (Compound 58)

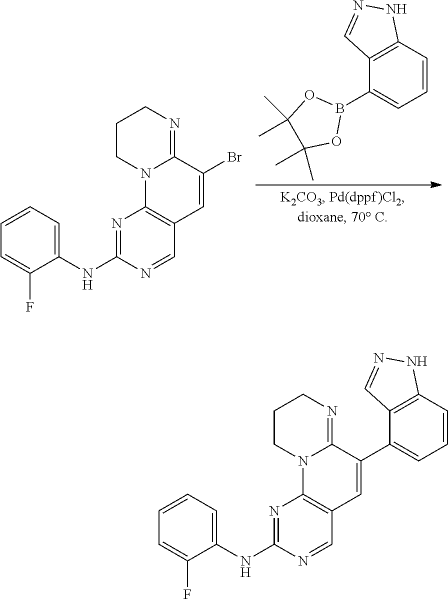

### Step 1: Synthesis of N-(2-fluorophenyl)-6-(1H-indazol-4-yl)-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (20 mg, 0.05 mmol), 4-(4,4,5,5-tetramethyl-1,3,2-dioxaborolan-2-yl)-1H-indazole (20 mg, 0.08 mmol), Pd(dppf)Cl₂ (5 mg) and K₂CO₃ (22 mg, 0.16 mmol) in dioxane (2 mL) was stirred at 70° C. for 2 hours. The mixture was purified by Prep-HPLC (0.1% formic acid, CH₃CN in water) to afford N-(2-fluorophenyl)-6-(1H-indazol-4-yl)-9,10-dihydro-8H-pyrido[1,6-a:2,3-d′]dipyrimidin-2-amine (4.1 mg, 19% yield) as a yellow solid. ¹H NMR (400 MHz, CD₃OD): δ 8.77 (s, 1H), 8.45 (s, 1H), 7.94 (s, 1H), 7.89-7.87 (m, 2H), 7.62 (d, J=8.4 Hz, 1H), 7.46 (t, J=8.0 Hz, 1H), 7.19-7.12 (m, 5H), 4.47 (t, J=5.2 Hz, 2H), 3.40 (t, J=5.2 Hz, 2H), 2.16-2.13 (m, 2H). LCMS (M+H⁺) m/z: 412.0.

### Example 59: Preparation of N-(2-fluorophenyl)-6-(5-methyl-1H-indazol-4-yl)-9,10-dihydro-8H-pyrido[1,6-a:2,3-d′]dipyrimidin-2-amine (Compound 59)

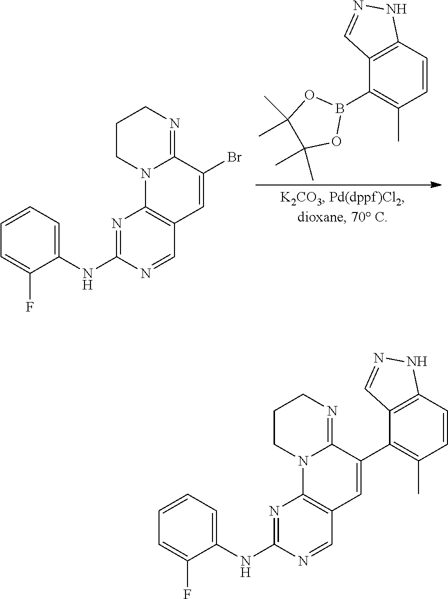

### Step 1: Synthesis of N-(2-fluorophenyl)-6-(5-methyl-1H-indazol-4-yl)-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (40 mg, 0.11 mmol), 5-methyl-4-(4,4,5,5-tetramethyl-1,3,2-dioxaborolan-2-yl)-1H-indazole (40 mg, 0.16 mmol), Pd(dppf)Cl₂ (5 mg) and K₂CO₃ (44 mg, 0.32 mmol) in dioxane (20 mL) was stirred at 70° C. for 2 hours. The mixture was purified by Prep-HPLC (0.1% NH₄HCO₃) twice to afford N-(2-fluorophenyl)-6-(5-methyl-1H-indazol-4-yl)-9,10-dihydro-8H-pyrido[1,6-a:2,3-d′]dipyrimidin-2-amine (7.7 mg, 17% yield) as a yellow solid. ¹H NMR (400 MHz, CD₃OD): δ 8.94 (s, 1H), 8.56 (s, 1H), 8.01-7.98 (m, 2H), 7.86 (s, 1H), 7.66 (d, J=8.8 Hz, 1H), 7.48 (d, J=8.8 Hz, 1H), 7.31-7.24 (m, 4H), 4.64-4.60 (m, 2H), 3.55-3.45 (m, 2H), 2.36 (s, 3H), 2.31-2.24 (s, 2H). LCMS (M+H⁺) m/z: 426.1.

### Example 60: Preparation of 6-(2-chlorophenyl)-N-(3-methoxyphenyl)-9,10-dihydro-8H-pyrido[1,6-a:2,3-d′]dipyrimidin-2-amine (Compound 60)

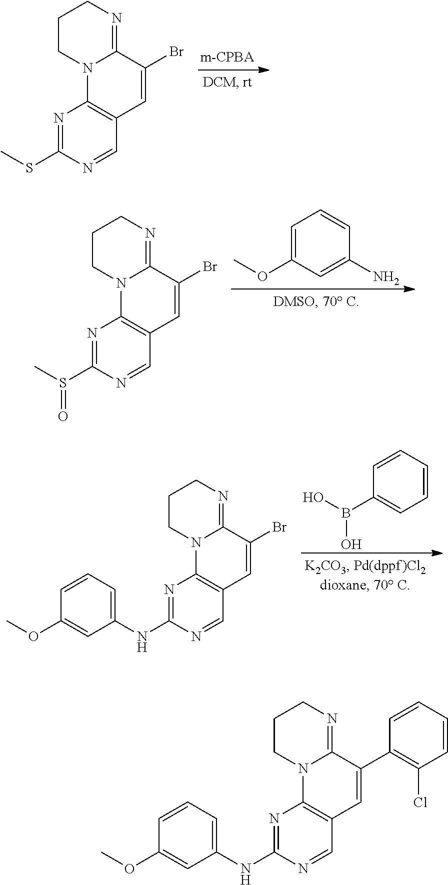

### Step 1: Synthesis of 6-bromo-2-(methylsulfinyl)-9,10-dihydro-8H-pyrido[1,6-a:2,3-d′]dipyrimidine

To a mixture of 6-bromo-2-(methylthio)-9,10-dihydro-8H-pyrido[1,6-a:2,3-d′]dipyrimidine (100 mg, 0.3 mmol) in DCM (4 mL) was added m-CPBA (165 mg, 0.9 mmol) at room temperature. The mixture was stirred for 1 hour at room temperature. The reaction mixture was concentrated to remove DCM to give crude 6-bromo-2-(methylsulfinyl)-9,10-dihydro-8H-pyrido[1,6-a:2,3-d′]dipyrimidine (242 mg) which was used to next step directly. LCMS (M+H⁺) m/z: 327.0.

### Step 2: Synthesis of 6-bromo-N-(3-methoxyphenyl)-9,10-dihydro-8H-pyrido[1,6-a:2,3-d′]dipyrimidin-2-amine

A mixture of 6-bromo-2-(methylsulfinyl)-9,10-dihydro-8H-pyrido[1,6-a:2,3-d′]dipyrimidine (242 mg, crude, 0.3 mmol), 3-methoxybenzenamine (184 mg, 1.5 mmol) in DMSO (4 mL) was stirred at 70° C. for 2 hours, and then detected by LCMS, reaction was worked 40%. The reaction mixture was purification by HPLC to give 6-bromo-N-(3-methoxyphenyl)-9,10-dihydro-8H-pyrido[1,6-a:2,3-d′]dipyrimidin-2-amine (26 mg, 22% yield over 2 steps). LCMS (M+H⁺) m/z: 386.0.

### Step 3: Synthesis of 6-(2-chlorophenyl)-N-(3-methoxyphenyl)-9,10-dihydro-8H-pyrido[1,6-a:2,3-d′]dipyrimidin-2-amine

A mixture of 6-bromo-N-(3-methoxyphenyl)-9,10-dihydro-8H-pyrido[1,6-a:2,3-d′]dipyrimidin-2-amine (24 mg, 0.06 mmol), 2-chlorophenylboronic acid (12 mg, 0.08 mmol), K₂CO₃ (25 mg, 0.18 mmol), and Pd(dppf)Cl₂ (5 mg, 0.006 mmol, 10 mol %) were suspended with THE (3 mL) and H₂O (0.5 mL) at protected by N₂, and the reaction mixture was refluxed for 3˜5 hours. Reaction solution was purified by Prep-HPLC (0.1% formic acid, CH₃CN in water) to give 6-(2-chlorophenyl)-N-(3-methoxyphenyl)-9,10-dihydro-8H-pyrido[1,6-a:2,3-d′]dipyrimidin-2-amine (3.6 mg, formic acid salt) as a yellow solid. ¹H NMR (400 MHz, DMSO-d₆): δ 9.81 (s, 1H), 8.41 (s, 1H), 8.26 (s, 1H), 7.59 (t, J=2.0 Hz, 1H), 7.48-7.45 (m, 1H), 7.36-7.28 (m, 4H), 7.20 (t, J=8.0 Hz, 1H), 7.14 (s, 1H), 6.57 (dd, J=8.0, 2.0, 1H), 4.18-4.16 (m, 2H), 3.76 (s, 3H), 3.38-3.37 (m, 2H), 1.90-1.86 (m, 2H). LCMS (M+H⁺) m/z: 418.1.

### Example 61: Preparation of 6-(2-chlorophenyl)-N-(2-methoxyphenyl)-9,10-dihydro-8H-pyrido[1,6-a:2,3-d′]dipyrimidin-2-amine (Compound 61)

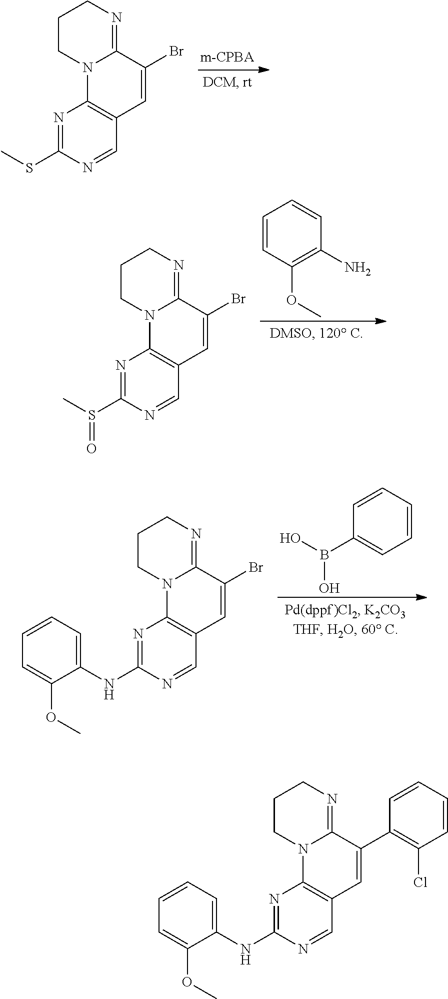

### Step 1: Synthesis of 6-bromo-2-(methylsulfinyl)-9,10-dihydro-8H-pyrido[1,6-a:2,3-d′]dipyrimidine

To a mixture of 6-bromo-2-(methylthio)-9,10-dihydro-8H-pyrido[1,6-a:2,3-d′]dipyrimidine (200 mg, 0.64 mmol) in DCM (5 mL) was added m-CPBA (332 mg, 1.93 mmol) at room temperature. The mixture was stirred at room temperature for 1 hour. The reaction mixture was concentrated to remove DCM to give crude 6-bromo-2-(methylsulfinyl)-9,10-dihydro-8H-pyrido[1,6-a:2,3-d′]dipyrimidine (230 mg) which was used to next step directly. LCMS (M+H⁺) m/z: 327.0.

### Step 2: Synthesis of 6-bromo-N-(2-methoxyphenyl)-9,10-dihydro-8H-pyrido[1,6-a:2,3-d′]dipyrimidin-2-amine

To a solution of 6-bromo-2-(methylsulfinyl)-9,10-dihydro-8H-pyrido[1,6-a:2,3-d′]dipyrimidine (230 mg, 0.7 mmol) in DMSO (4 mL) was added 2-methoxyaniline (129 mg, 1.05 mmol). The mixture was stirred at 120° C. for 2 hours. The mixture was extracted with EtOAc (10 mL×2) and the combined organic layers were washed with brine (10 mL), dried over anhydrous Na₂SO₄, filtered, concentrated in vacuum to give crude product which was purified on silica gel column flash chromatography (DCM/MeOH=1/1, v/v) to afford 6-bromo-N-(2-methoxyphenyl)-9,10-dihydro-8H-pyrido[1,6-a:2,3-d′]dipyrimidin-2-amine (43 mg, 15.9% yield) as a yellow oil. LCMS (M+H⁺) m/z: 385.9.

### Step 3: Synthesis of 6-(2-chlorophenyl)-N-(2-methoxyphenyl)-9,10-dihydro-8H-pyrido[1,6-a:2,3-d′]dipyrimidin-2-amine

To a solution of 6-bromo-N-(2-methoxyphenyl)-9,10-dihydro-8H-pyrido[1,6-a:2,3-d′]dipyrimidin-2-amine (20 mg, 0.05 mmol) in THF (2 mL) was added (2-chlorophenyl)boronic acid (10 mg, 0.06 mmol), Pd(dppf)Cl₂ (2 mg, 0.002 mmol), K₂CO₃ (20 mg, 0.15 mmol) and H₂O (0.2 mL). The mixture was stirred at 60° C. for 2 hours. The mixture was extracted with EtOAc (10 mL×2) and the combined organic layers were washed with brine (10 mL), dried over anhydrous Na₂SO₄, filtered, concentrated in vacuum to give crude product which was purified by prep-HPLC (0.1% formic acid, CH₃CN in water) to afford 6-(2-chlorophenyl)-N-(2-methoxyphenyl)-9,10-dihydro-8H-pyrido[1,6-a:2,3-d′]dipyrimidin-2-amine (4.2 mg, 20% yield, formic acid salt) as a yellow solid. ¹H NMR (400 MHz, DMSO-d₆): δ 8.42 (s, 1H), 8.35 (s, 1H), 8.23 (s, 1H), 8.13 (d, J=8.0 Hz, 1H), 7.49-7.46 (m, 1H), 7.38-7.35 (m, 2H), 7.34-7.30 (m, 1H), 7.19 (s, 1H), 7.08-7.06 (m, 2H), 7.01-6.97 (m, 1H), 4.13-4.10 (m, 2H), 3.87 (s, 3H), 2.46-2.44 (m, 2H), 1.89-1.86 (m, 2H). LCMS (M+H⁺) m/z: 418.0.

### Example 62: Preparation of N-(2-methoxyphenyl)-6-(5-methyl-1H-indazol-4-yl)-9,10-dihydro-8H-pyrido[1,6-a:2,3-d′]dipyrimidin-2-amine (Compound 62)

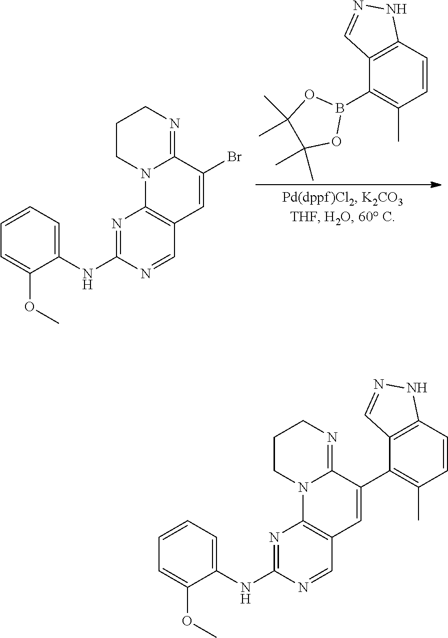

### Step 1: Synthesis of N-(2-methoxyphenyl)-6-(5-methyl-1H-indazol-4-yl)-9,10-dihydro-8H-pyrido[1,6-a:2,3-d′]dipyrimidin-2-amine

To a solution of 6-bromo-N-(2-methoxyphenyl)-9,10-dihydro-8H-pyrido[1,6-a:2,3-d′]dipyrimidin-2-amine (23 mg, 0.06 mmol) in THF (2 mL) was added 5-methyl-4-(4,4,5,5-tetramethyl-1,3,2-dioxaborolan-2-yl)-1H-indazole (18 mg, 0.07 mmol), Pd(dppf)Cl₂ (3 mg, 0.003 mmol), K₂CO₃ (25 mg, 0.18 mmol) and H₂O (0.2 mL). The mixture was stirred at 60° C. for 16 hours. The mixture was extracted with EtOAc (10 mL×2) and the combined organic layers were washed with brine (10 mL), dried over anhydrous Na₂SO₄, filtered, concentrated in vacuum to give crude product which was purified by prep-HPLC (0.1% formic acid, CH₃CN in water) to afford N-(2-methoxyphenyl)-6-(5-methyl-1H-indazol-4-yl)-9,10-dihydro-8H-pyrido[1,6-a:2,3-d′]dipyrimidin-2-amine (5.1 mg, 20% yield, formic acid salt) as a yellow solid. ¹H NMR (400 MHz, CD₃OD): δ 8.82 (s, 1H), 8.44 (s, 1H), 8.17-8.15 (m, 1H), 7.88 (s, 1H), 7.74 (s, 1H), 7.55 (d, J=8.8 Hz, 1H), 7.37 (d, J=8.8 Hz, 1H), 7.10-6.94 (m, 3H), 4.58-4.54 (m, 2H), 3.86 (s, 3H), 3.41-3.36 (m, 2H), 2.24-2.16 (m, 5H). LCMS (M+H⁺) m/z: 438.1.

### Example 63: Preparation of N-(2-methoxypyridin-3-yl)-6-(5-methyl-1H-indazol-4-yl)-9,10-dihydro-8H-pyrido[1,6-a:2,3-d′]dipyrimidin-2-amine (Compound 63)

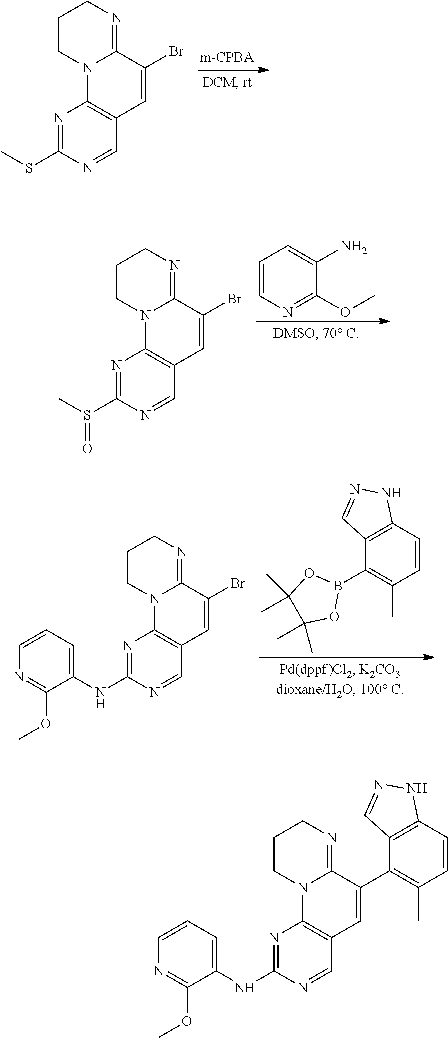

### Step 1: Synthesis of 6-bromo-2-(methylsulfinyl)-9,10-dihydro-8H-pyrido[1,6-a:2,3-d′]dipyrimidine

A solution of 6-bromo-2-(methylthio)-9,10-dihydro-8H-pyrido[1,6-a:2,3-d′]dipyrimidine (100 mg, 0.32 mmol), m-CPBA (61 mg, 0.35 mmol) in DCM (20 mL) was stirred at room temperature for 30 minutes. After concentration at room temperature, the residue, 6-bromo-2-(methylsulfinyl)-9,10-dihydro-8H-pyrido[1,6-a:2,3-d′]dipyrimidine was used into next step without further purification. LCMS (M+H⁺) m/z: 326.5.

### Step 2: Synthesis of 6-bromo-N-(2-methoxypyridin-3-yl)-9,10-dihydro-8H-pyrido[1,6-a:2,3-d′]dipyrimidin-2-amine

To a solution of 6-bromo-2-(methylsulfinyl)-9,10-dihydro-8H-pyrido[1,6-a:2,3-d′]dipyrimidine (60 mg, 0.18 mmol) in DMSO (5 mL) was added 2-methoxypyridin-3-amine (34 mg, 0.27 mmol). The reaction mixture was stirred at 70° C. for 16 hours. The reaction solution was purified by Prep-HPLC (0.1% formic acid, CH₃CN in water) to give 6-bromo-N-(2-methoxypyridin-3-yl)-9,10-dihydro-8H-pyrido[1,6-a:2,3-d′]dipyrimidin-2-amine (20 mg, 16% yield over 2 steps). LCMS (M+H⁺) m/z: 387.1.

### Step 3: Synthesis of N-(2-methoxypyridin-3-yl)-6-(5-methyl-1H-indazol-4-yl)-9,10-dihydro-8H-pyrido[1,6-a:2,3-d′]dipyrimidin-2-amine

To a solution of 6-bromo-N-(2-methoxypyridin-3-yl)-9,10-dihydro-8H-pyrido[1,6-a:2,3-d′]dipyrimidin-2-amine (20 mg, 0.05 mmol), 5-methyl-4-(4,4,5,5-tetramethyl-1,3,2-dioxaborolan-2-yl)-1H-indazole (20 mg, 0.08 mmol) and K₂CO₃ (21 mg, 0.15 mmol) in mixed solvent of dioxane (3 mL) and H₂O (0.3 mL), then was added Pd(dppf)Cl₂ (4 mg, 0.01 mmol). After addition and de-gas under nitrogen gas, then the reaction mixture was stirred at 100° C. for 16 hours. The reaction solution was purified directly by Prep-HPLC to give product, which was not pure enough. Further purification was also purified again by Prep-HPLC (0.1% formic acid, CH₃CN in water) to give N-(2-methoxypyridin-3-yl)-6-(5-methyl-1H-indazol-4-yl)-9,10-dihydro-8H-pyrido[1,6-a:2,3-d′]dipyrimidin-2-amine (1.5 mg, 100% purity, formic acid salt) as a yellow solid. ¹H NMR (400 MHz, CD₃OD): δ 9.00 (s, 1H), 8.59 (dd, J=7.6, 1.2 Hz, 1H), 8.52 (s, 1H), 8.06 (s, 1H), 7.95 (dd, J=4.8, 1.2 Hz, 1H), 7.85 (s, 1H), 7.67 (d, J=8.4 Hz, 1H), 7.48 (d, J=8.8 Hz, 1H), 7.09 (dd, J=7.6, 4.8 Hz, 1H), 4.70-4.68 (m, 2H), 4.07 (s, 3H), 3.53-3.51 (m, 2H), 2.35-2.32 (m, 5H). LCMS (M+H⁺) m/z: 439.1.

### Example 64: Preparation of 3-methyl-1-(4-(2-(methylamino)-9,10-dihydro-8H-pyrido[1,6-a:2,3-d′]dipyrimidin-6-yl)phenyl)pyrazin-2(1H)-one (Compound 64)

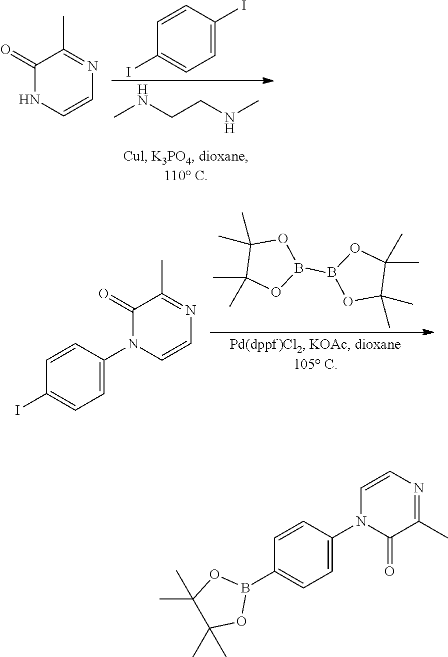

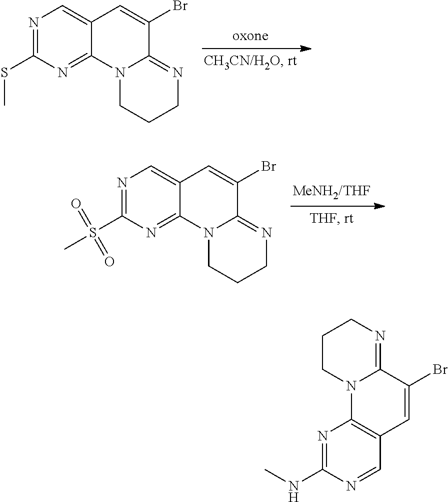

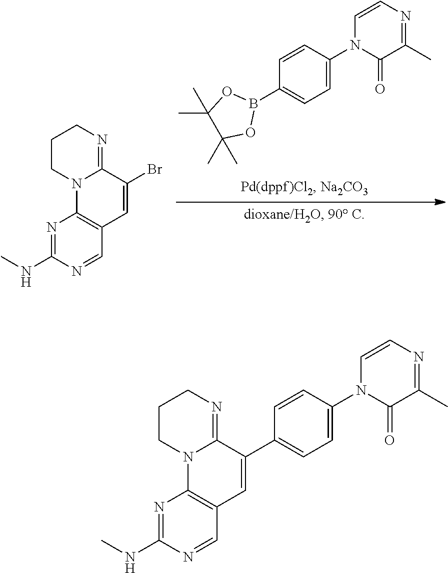

### Step 1: Synthesis of 1-(4-iodophenyl)-3-methylpyrazin-2(1H)-one

To a solution of 3-methylpyrazin-2(1H)-one (500 mg, 4.54 mmol) in dioxane (15 mL) was added CuI (130 mg, 0.68 mmol), K₃PO₄ (962 mg, 4.54 mmol), N¹,N²-dimethylethane-1,2-diamine (120 mg, 1.36 mmol) and 1,4-diiodobenzene (643 mg, 2.27 mmol) at room temperature purged and was degassed with N₂ for 3 times, the mixture was stirred at 110° C. for 18 hours. The solvent was removed and purified by column chromatography on silica gel (PE/EtOAc=3:1) to afford 1-(4-iodophenyl)-3-methylpyrazin-2(1H)-one (280 mg, 47% yield). LCMS (M+H⁺) m/z: 313.0

### Step 2: Synthesis of 3-methyl-1-(4-(4,4,5,5-tetramethyl-1,3,2-dioxaborolan-2-yl)phenyl)pyrazin-2(1H)-one

To a solution of 1-(4-iodophenyl)-3-methylpyrazin-2(1H)-one (280 mg, 1.05 mmol) in dioxane (15 mL) was added Pd(dppf)Cl₂ (150 mg, 0.205 mmol), KOAc (300 mg, 3.1 mmol) and 4,4,4′,4′,5,5,5′,5′-octamethyl-2,2′-bi(1,3,2-dioxaborolane) (536 mg, 2.1 mmol) at room temperature purged and was degassed with N₂ for 3 times, the mixture was stirred at 105° C. for 18 hours. The solvent was removed and purified by column chromatography on silica gel (PE/EtOAc=3:1) to afford 3-methyl-1-(4-(4,4,5,5-tetramethyl-1,3,2-dioxaborolan-2-yl)phenyl)pyrazin-2(1H)-one (250 mg, 76% yield). LCMS (M+H⁺) m/z: 313.3

### Step 3: Synthesis of 6-bromo-2-(methylsulfonyl)-9,10-dihydro-8H-pyrido[1,6-a:2,3-d′]dipyrimidine

A mixture of 6-bromo-2-(methylthio)-9,10-dihydro-8H-pyrido[1,6-a:2,3-d′]dipyrimidine (700 mg, 2.2 mmol) and oxone (3.47 g, 5.65 mmol) in CH₃CN (10 mL) and water (10 mL) was stirred at room temperature for 2 hours. The reaction mixture was concentrated in vacuum and purified by prep-HPLC (0.1% TFA, CH₃CN in H₂O) to afford 6-bromo-2-(methylsulfonyl)-9,10-dihydro-8H-pyrido[1,6-a:2,3-d′]dipyrimidine (crude, 1.1 g). LCMS (M+H⁺) m/z: 343.0

### Step 4: Synthesis of 6-bromo-N-methyl-9,10-dihydro-8H-pyrido[1,6-a:2,3-d′]dipyrimidin-2-amine

To a mixture of 6-bromo-2-(methylsulfonyl)-9,10-dihydro-8H-pyrido[1,6-a:2,3-d′]dipyrimidine (1.1 g, 3.2 mmol) in THF (10 mL) was added MeNH₂ in THF (5 mL, 2M) and the mixture was stirred at room temperature for 2 hours. The solvent was removed and the residue was purified by column chromatography on silica gel (DCM/MeOH=20:1) to afford 6-bromo-N-methyl-9,10-dihydro-8H-pyrido[1,6-a:2,3-d′]dipyrimidin-2-amine (220 mg, 23% yield) as a yellow solid. LCMS (M+H⁺) m/z: 294.0

### Step 5: Synthesis of 3-methyl-1-(4-(2-(methylamino)-9,10-dihydro-8H-pyrido[1,6-a:2,3-d′]dipyrimidin-6-yl)phenyl)pyrazin-2(1H)-one

To a solution of 6-bromo-N-methyl-9,10-dihydro-8H-pyrido[1,6-a:2,3-d′]dipyrimidin-2-amine (40 mg, 0.136 mmol) in dioxane/H₂O (5 mL) was added Pd(dppf)Cl₂ (10 mg, 0.013 mmol), Na₂CO₃ (30 mg, 0.272 mmol) and 3-methyl-1-(4-(4,4,5,5-tetramethyl-1,3,2-dioxaborolan-2-yl)phenyl)pyrazin-2(1H)-one (51 mg, 0.168 mmol) at room temperature purged and was degassed with N₂ for 3 times, the mixture was stirred at 90° C. for 18 hours. The mixture was diluted with water (50 mL) and then extracted with EtOAc (50 mL×3). The reaction mixture was concentrated in vacuum to remove solvent and then purified on prep-HPLC (0.1% TFA, CH₃CN in H₂O) to afford 3-methyl-1-(4-(2-(methylamino)-9,10-dihydro-8H-pyrido[1,6-a:2,3-d′]dipyrimidin-6-yl)phenyl)pyrazin-2(1H)-one (10 mg, 18% yield, TFA salt) as a yellow solid. ¹H NMR (400 MHz, CD₃OD): δ 8.77 (s, 1H), 7.94 (s, 1H), 7.67 (s, 4H), 7.47 (d, J=4.4 Hz, 1H), 7.35 (d, J=4.8 Hz, 1H), 4.69-4.66 (m, 2H), 3.60-3.57 (m, 2H), 3.09 (s, 3H), 2.47 (s, 3H), 2.31-2.28 (m, 2H). LCMS (M+H⁺) m/z: 400.1.

### Example 65: Preparation of 3-(2-((2-fluorophenyl)amino)-9,10-dihydro-8H-pyrido[1,6-a:2,3-d′]dipyrimidin-6-yl)-4-methylphenol (Compound 65)

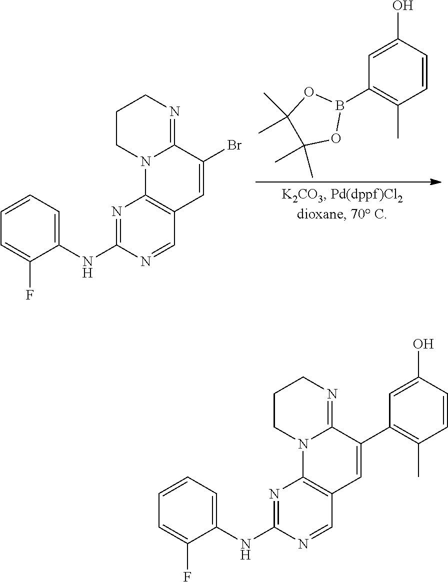

### Step 1: Synthesis of 3-(2-((2-fluorophenyl)amino)-9,10-dihydro-8H-pyrido[1,6-a:2,3-d′]dipyrimidin-6-yl)-4-methylphenol

A solution of 6-bromo-N-(2-fluorophenyl)-9,10-dihydro-8H-pyrido[1,6-a:2,3-d′]dipyrimidin-2-amine (30 mg, 0.08 mmol), 4-methyl-3-(4,4,5,5-tetramethyl-1,3,2-dioxaborolan-2-yl)phenol (28 mg, 0.12 mmol), Pd(dppf)Cl₂ (5 mg) and K₂CO₃ (33 mg, 0.24 mmol) in dioxane (2 mL) was stirred at 70° C. for 2 hours. The mixture was purified directly by Prep-HPLC (0.1% NH₄HCO₃, CH₃CN in H₂O) twice to afford 3-(2-((2-fluorophenyl)amino)-9,10-dihydro-8H-pyrido[1,6-a:2,3-d′]dipyrimidin-6-yl)-4-methylphenol (7.0 mg, 22% yield) as a yellow solid. ¹H NMR (400 MHz, DMSO-d₆): δ 9.18 (s, 1H), 9.07 (s, 1H), 8.30 (s, 1H), 7.84-7.80 (m, 1H), 7.27-7.13 (m, 3H), 6.97 (s, 1H), 6.93 (d, J=8.0 Hz, 1H), 6.59 (dd, J=8.0, 2.4 Hz, 1H), 6.50 (d, J=2.8 Hz, 1H), 4.01 (t, J=6.0 Hz, 2H), 3.37 (t, J=5.2 Hz, 2H), 2.02 (s, 3H), 1.84-1.80 (m, 2H). LCMS (M+H⁺) m/z: 402.0.

### Example 66: Preparation of N-ethyl-6-(5-methyl-1H-indazol-4-yl)-9,10-dihydro-8H-pyrido[1,6-a:2,3-d′]dipyrimidin-2-amine (Compound 66)

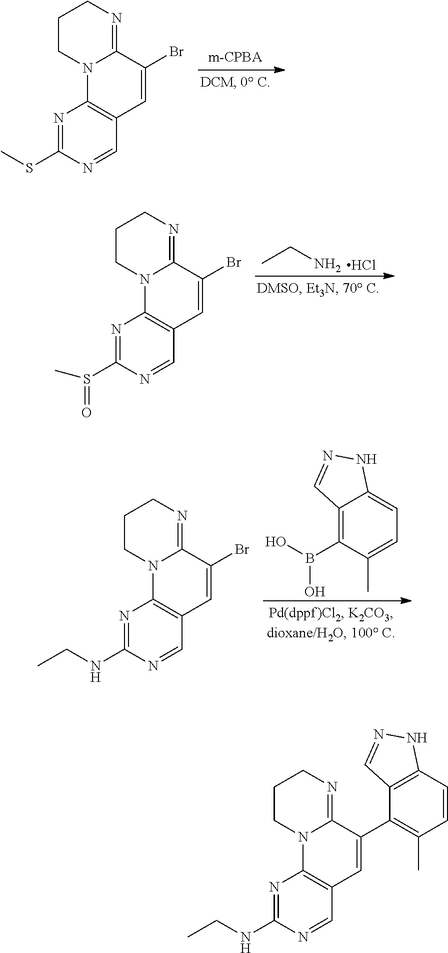

### Step 1: Synthesis of 6-bromo-2-(methylsulfinyl)-9,10-dihydro-8H-pyrido[1,6-a:2,3-d′]dipyrimidine

To a solution of 6-bromo-2-(methylthio)-9,10-dihydro-8H-pyrido[1,6-a:2,3-d′]dipyrimidine (100 mg, 0.3 mmol) in DCM (4 mL) was added m-CPBA (163 mg, 0.9 mmol). The mixture was stirred for 3 hours at 0° C. The reaction mixture was concentrated to remove DCM to give 6-bromo-2-(methylsulfinyl)-9,10-dihydro-8H-pyrido[1,6-a:2,3-d′]dipyrimidine (crude, 242 mg) which was used to next step directly. LCMS (M+H⁺) m/z: 327.0.

### Step 2: Synthesis of 6-bromo-N-ethyl-9,10-dihydro-8H-pyrido[1,6-a:2,3-d′]dipyrimidin-2-amine

A mixture of 6-bromo-2-(methylsulfinyl)-9,10-dihydro-8H-pyrido[1,6-a:2,3-d′]dipyrimidine (242 mg, crude), ethanamine hydrochloride (58 mg, 0.9 mmol), Et₃N (165 mg, 1.5 mmol) in DMSO (4 mL) was stirred at 70° C. for 3 hours. The reaction mixture was purified by Prep-HPLC (0.1% NH₄HCO₃, CH₃CN in H₂O) to give 6-bromo-N-ethyl-9,10-dihydro-8H-pyrido[1,6-a:2,3-d′]dipyrimidin-2-amine (38 mg, 41% yield). LCMS (M+H⁺) m/z: 308.0.

### Step 3: Synthesis of N-ethyl-6-(5-methyl-1H-indazol-4-yl)-9,10-dihydro-8H-pyrido[1,6-a:2,3-d′]dipyrimidin-2-amine

6-Bromo-N-ethyl-9,10-dihydro-8H-pyrido[1,6-a:2,3-d′]dipyrimidin-2-amine (28 mg, 0.09 mmol), 5-methyl-1H-indazol-4-ylboronic acid (19 mg, 0.12 mmol), K₂CO₃ (37 mg, 0.27 mmol), and Pd(dppf)Cl₂ (7 mg, 10 mol %) were suspended with 1,4-dioxane (2 mL) and H₂O (0.5 mL) under N₂. The reaction mixture was refluxed for 3˜5 hours. The reaction mixture was purified by Prep-HPLC (0.1% NH₄HCO₃, CH₃CN in H₂O) to give N-ethyl-6-(5-methyl-1H-indazol-4-yl)-9,10-dihydro-8H-pyrido[1,6-a:2,3-d′]dipyrimidin-2-amine (4.9 mg, 15% yield) as a pale yellow solid. ¹H NMR (400 MHz, CD₃OD): δ 8.36 (s, 1H), 7.76 (s, 1H), 7.48 (d, J=8.4 Hz, 1H), 7.35 (s, 1H), 7.33 (s, 1H), 4.40-4.34 (m, 2H), 3.54-3.48 (m, 2H), 3.44-3.39 (m, 2H), 2.30 (s, 3H), 2.09-2.05 (m, 2H), 1.27 (t, J=7.2 Hz, 3H). LCMS (M+H⁺) m/z: 360.1.

### Example 67: Preparation of 3-((6-(2-chlorophenyl)-9,10-dihydro-8H-pyrido[1,6-a:2,3-d′]dipyrimidin-2-yl)amino)pyridin-4-ol (Compound 67)

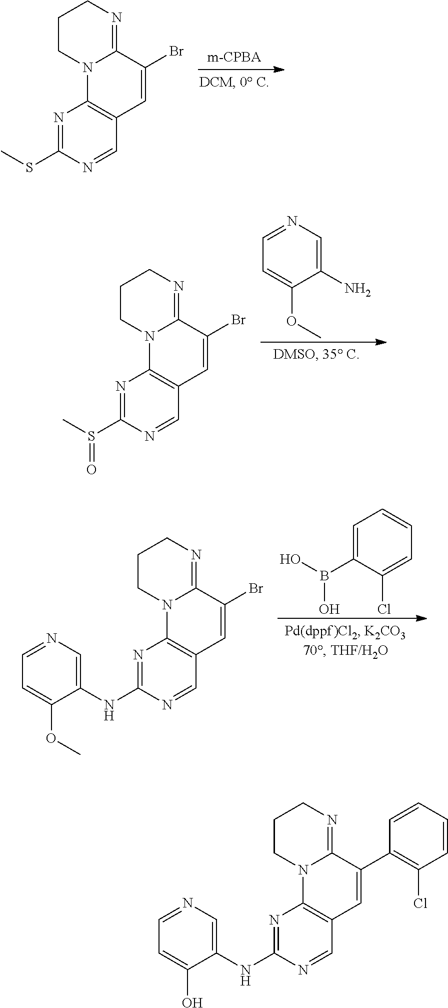

### Step 1: Synthesis of 6-bromo-2-(methylsulfinyl)-9,10-dihydro-8H-pyrido[1,6-a:2,3-d′]dipyrimidine

To a solution of 6-bromo-2-(methylthio)-9,10-dihydro-8H-pyrido[1,6-a:2,3-d′]dipyrimidine (150 mg, 0.5 mmol) in DCM (4 mL) was added m-CPBA (130 mg, 0.75 mmol). The mixture was stirred for 1 hour at 0° C. The reaction mixture was concentrated to remove DCM to give 6-bromo-2-(methylsulfinyl)-9,10-dihydro-8H-pyrido[1,6-a:2,3-d′]dipyrimidine (crude, 290 mg) which was used to next step directly. LCMS (M+H⁺) m/z: 327.0.

### Step 2: Synthesis of 6-bromo-N-(4-methoxypyridin-3-yl)-9,10-dihydro-8H-pyrido[1,6-a:2,3-d′]dipyrimidin-2-amine

A mixture of 6-bromo-2-(methylsulfinyl)-9,10-dihydro-8H-pyrido[1,6-a:2,3-d′]dipyrimidine (290 mg, crude, 0.5 mmol), 4-methoxypyridin-3-amine (186 mg, 1.5 mmol) in DMSO (4 mL) was stirred at 35° C. for 0.5 hour. The reaction mixture was purified by prep-HPLC (0.1% NH₄HCO₃, CH₃CN in H₂O) to give 6-bromo-N-(4-methoxypyridin-3-yl)-9,10-dihydro-8H-pyrido[1,6-a:2,3-d′]dipyrimidin-2-amine (42 mg, yield 22% of two steps). LCMS (M+H⁺) m/z: 387.0.

### Step 3: Synthesis of 3-((6-(2-chlorophenyl)-9,10-dihydro-8H-pyrido[1,6-a:2,3-d′]dipyrimidin-2-yl)amino)pyridin-4-ol

6-Bromo-N-(4-methoxypyridin-3-yl)-9,10-dihydro-8H-pyrido[1,6-a:2,3-d′]dipyrimidin-2-amine (24 mg, 0.06 mmol), (2-chlorophenyl)boronic acid (12 mg, 0.08 mmol), K₂CO₃ (25 mg, 0.18 mmol), and Pd(dppf)Cl₂ (5 mg, 10 mol %) were suspended with THE (2 mL) and H₂O (0.5 mL) at protected by N₂. The reaction mixture was refluxed for 3˜5 hours. The reaction mixture was purified by prep-HPLC (0.1% NH₄HCO₃, CH₃CN in H₂O) to give 3-((6-(2-chlorophenyl)-9,10-dihydro-8H-pyrido[1,6-a:2,3-d′]dipyrimidin-2-yl)amino)pyridin-4-ol (8.5 mg, 34% yield) as a pale yellow solid. ¹H NMR (400 MHz, CD₃OD): δ 8.95 (dd, J=7.6, 2.4 Hz, 1H), 8.74 (d, J=2.4 Hz, 1H), 8.65 (s, 1H), 7.49-7.46 (m, 1H), 7.39-7.29 (m, 4H), 6.46 (d, J=7.6 Hz, 1H), 4.40-4.35 (m, 2H), 3.54-3.50 (m, 2H), 2.08-2.04 (m, 2H). LCMS (M+H⁺) m/z: 405.

### Example 68: Preparation of 6-(2,4-dichlorophenyl)-N-(2-fluoro-4-(piperazin-1-yl)phenyl)-9,10-dihydro-8H-pyrido[1,6-a:2,3-d′]dipyrimidin-2-amine (Compound 68)

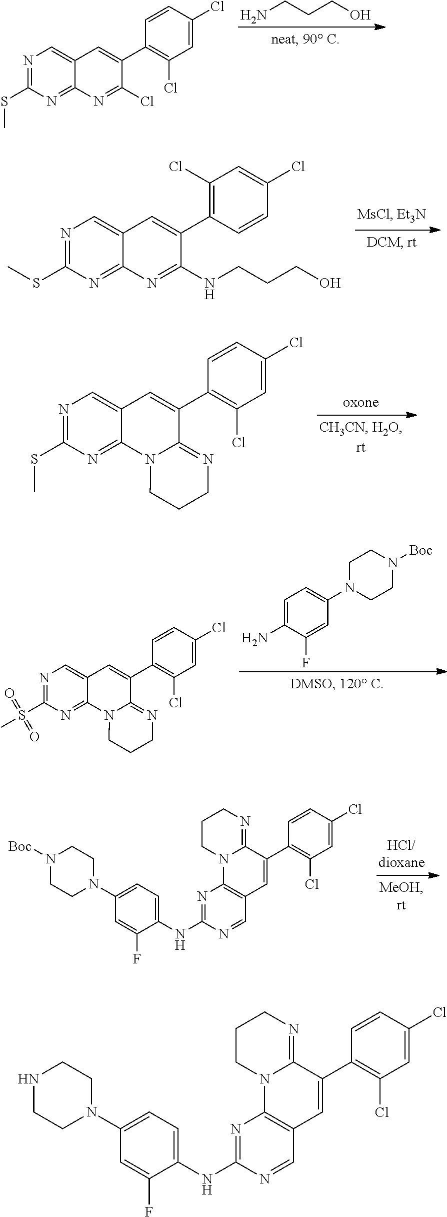

### Step 1: Synthesis of 3-((6-(2,4-dichlorophenyl)-2-(methylthio)pyrido[2,3-d]pyrimidin-7-yl)amino)propan-1-ol

A solution of 7-chloro-6-(2,4-dichlorophenyl)-2-(methylthio)pyrido[2,3-d]pyrimidine (5.5 g, 15.4 mmol) and 3-aminopropan-1-ol (20 mL) was stirred at 90° C. for 2 hours under N₂. The mixture was added water (100 mL), filtered to afford 3-((6-(2,4-dichlorophenyl)-2-(methylthio)pyrido[2,3-d]pyrimidin-7-yl)amino)propan-1-ol (6.8 g, crude) as a yellow solid. LCMS (M+H⁺) m/z: 395.1.

### Step 2: Synthesis of 6-(2,4-dichlorophenyl)-2-(methylthio)-9,10-dihydro-8H-pyrido[1,6-a:2,3-d′]dipyrimidine

To a solution of 3-((6-(2,4-dichlorophenyl)-2-(methylthio)pyrido[2,3-d]pyrimidin-7-yl)amino)propan-1-ol (6.7 g, 16.9 mmol) and Et₃N (5.1 g, 7.86 mmol) in DCM (70 mL) was added MsCl (2.91 g, 25.3 mmol). The reaction mixture was stirred at room temperature for 16 hours under N₂. The mixture was concentrated in vacuum and purified by column chromatography on silica gel (DCM/MeOH=20:1) to afford 6-(2,4-dichlorophenyl)-2-(methylthio)-9,10-dihydro-8H-pyrido[1,6-a:2,3-d′]dipyrimidine (9.2 g, crude) as a yellow solid. LCMS (M+H⁺) m/z: 377.1.

### Step 3: Synthesis of 6-(2,4-dichlorophenyl)-2-(methylsulfonyl)-9,10-dihydro-8H-pyrido[1,6-a:2,3-d′]dipyrimidine

To a solution of 6-(2,4-dichlorophenyl)-2-(methylthio)-9,10-dihydro-8H-pyrido[1,6-a:2,3-d′]dipyrimidine (2.0 g, 5.30 mmol) in CH₃CN (10 mL) and H₂O (10 mL) was added oxone (3.2 g, 5.30 mmol). The reaction mixture was stirred at room temperature under N₂ for 2 hours. The mixture was purified by pre-HPLC (0.1% HCl) to afford 6-(2,4-dichlorophenyl)-2-(methylsulfonyl)-9,10-dihydro-8H-pyrido[1,6-a:2,3-d′]dipyrimidine (500 mg, 23% yield) as a white solid. LCMS (M+H⁺) m/z: 409.1.

### Step 4: Synthesis of tert-butyl 4-(4-((6-(2,4-dichlorophenyl)-9,10-dihydro-8H-pyrido[1,6-a:2,3-d′]dipyrimidin-2-yl)amino)-3-fluorophenyl)piperazine-1-carboxylate

To a solution of 6-(2,4-dichlorophenyl)-2-(methylsulfonyl)-9,10-dihydro-8H-pyrido[1,6-a:2,3-d′]dipyrimidine (150 mg, 0.36 mmol) in DMSO (3 mL) was added tert-butyl 4-(4-amino-3-fluorophenyl)piperazine-1-carboxylate (86 mg, 0.29 mmol). The reaction mixture was stirred at 120° C. for 2 hours under N₂. The mixture was added to water, extracted with EtOAc (10 mL×3), washed with brine (50 mL), dried over anhydrous Na₂SO₄, concentrated and purified by prep-TLC (MeOH/DCM=1:20) to afford tert-butyl 4-(4-((6-(2,4-dichlorophenyl)-9,10-dihydro-8H-pyrido[1,6-a:2,3-d′]dipyrimidin-2-yl)amino)-3-fluorophenyl)piperazine-1-carboxylate (10 mg, 4% yield) as a yellow solid. LCMS (M+H⁺) m/z: 624.3.

### Step 5: Synthesis of 6-(2,4-dichlorophenyl)-N-(2-fluoro-4-(piperazin-1-yl)phenyl)-9,10-dihydro-8H-pyrido[1,6-a:2,3-d′]dipyrimidin-2-amine

To a solution of tert-butyl 4-(4-((6-(2,4-dichlorophenyl)-9,10-dihydro-8H-pyrido[1,6-a:2,3-d′]dipyrimidin-2-yl)amino)-3-fluorophenyl)piperazine-1-carboxylate (10 mg, 0.016 mmol) in MeOH (1 mL) was added HCl/dioxane (2 mL, 3M). The mixture was stirred at room temperature for 2 hours under N₂. The residue was concentrated in vacuum and purified by prep-HPLC (0.1% TFA, CH₃CN in H₂O) to give 6-(2,4-dichlorophenyl)-N-(2-fluoro-4-(piperazin-1-yl)phenyl)-9,10-dihydro-8H-pyrido[1,6-a:2,3-d′]dipyrimidin-2-amine (4.5 mg, 54% yield, TFA salt) as a yellow solid. ¹H NMR (400 MHz, DMSO-d₆): δ 8.96 (br, 1H), 8.05-8.03 (m, 1H), 7.89-7.87 (m, 1H), 7.65 (d, J=8.0 Hz, 1H), 7.52-7.47 (m, 2H), 6.99 (d, J=14.0 Hz, 1H), 6.88 (d, J=8.8 Hz, 1H), 4.44-4.26 (m, 2H), 3.46-3.43 (m, 6H), 3.26-3.25 (m, 4H), 2.20-2.05 (m, 2H). LCMS (M+H⁺) m/z: 524.2.

### Example 69: Preparation of 6-(2,4-dichlorophenyl)-N-(3-fluoro-4-(piperazin-1-yl)phenyl)-9,10-dihydro-8H-pyrido[1,6-a:2,3-d′]dipyrimidin-2-amine (Compound 69)

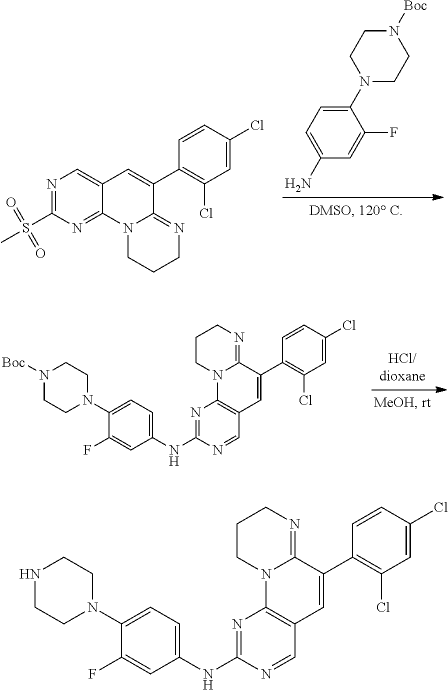 [SMILES: CS(=O)(=O)c1ncc2c(n1)N1CCCN=C1C(c1ccc(Cl)cc1Cl)=C2 ; CC(C)(C)OC(=O)N1CCN(c2ccc(N)cc2F)CC1 ; CC(C)(C)OC(=O)N1CCN(c2ccc(Nc3ncc4c(n3)N3CCCN=C3C(c3ccc(Cl)cc3Cl)=C4)cc2F)CC1 ; Fc1cc(Nc2ncc3c(n2)N2CCCN=C2C(c2ccc(Cl)cc2Cl)=C3)ccc1N1CCNCC1]

### Step 1: Synthesis of tert-butyl 4-(4-((6-(2,4-dichlorophenyl)-9,10-dihydro-8H-pyrido[1,6-a:2,3-d′]dipyrimidin-2-yl)amino)-2-fluorophenyl)piperazine-1-carboxylate

To a solution of 6-(2,4-dichlorophenyl)-2-(methylsulfonyl)-9,10-dihydro-8H-pyrido[1,6-a:2,3-d′]dipyrimidine (170 mg, 0.42 mmol) in DMSO (4 mL) was added tert-butyl 4-(4-amino-2-fluorophenyl)piperazine-1-carboxylate (122 mg, 0.42 mmol). The reaction mixture was stirred at 120° C. for 2 hours under N₂. The mixture was added to water, extracted with EtOAc (10 mL×3), washed with brine (50 mL), dried over anhydrous Na₂SO₄, concentrated and purified by pre-TLC (DCM/MeOH=20:1) to afford tert-butyl 4-(4-((6-(2,4-dichlorophenyl)-9,10-dihydro-8H-pyrido[1,6-a:2,3-d′]dipyrimidin-2-yl)amino)-2-fluorophenyl)piperazine-1-carboxylate (10 mg, 4% yield) as a black solid. LCMS (M+H⁺) m/z: 624.1.

### Step 2: Synthesis of 6-(2,4-dichlorophenyl)-N-(3-fluoro-4-(piperazin-1-yl)phenyl)-9,10-dihydro-8H-pyrido[1,6-a:2,3-d′]dipyrimidin-2-amine

To a solution of tert-butyl 4-(4-((6-(2,4-dichlorophenyl)-9,10-dihydro-8H-pyrido[1,6-a:2,3-d′]dipyrimidin-2-yl)amino)-2-fluorophenyl)piperazine-1-carboxylate (10 mg, 0.016 mmol) in MeOH (1 mL), was added HCl/dioxane (3 mL, 3M). The mixture was stirred at room temperature for 2 hours under N₂. The residue was concentrated and purified by Prep-HPLC (0.1% TFA, CH₃CN in H₂O) to give 6-(2,4-dichlorophenyl)-N-(3-fluoro-4-(piperazin-1-yl)phenyl)-9,10-dihydro-8H-pyrido[1,6-a:2,3-d′]dipyrimidin-2-amine (8.1 mg, 67% yield, TFA salt) as a yellow solid. ¹H NMR (400 MHz, DMSO-d₆): δ 9.04 (s, 1H), 8.07 (s, 1H), 7.87 (t, J=2.0 Hz, 1H), 7.72 (d, J=14.8 Hz, 1H), 7.65 (d, J=8.4 Hz, 1H), 7.60-7.57 (m, 1H), 7.54-7.52 (m, 1H), 7.17 (t, J=9.2 Hz, 1H), 4.62-4.46 (m, 2H), 3.49-3.45 (m, 2H), 3.28-3.22 (m, 8H), 2.29-2.10 (m, 2H). LCMS (M+H⁺) m/z: 524.2.

### Example 70: Preparation of 6-(2,4-dichlorophenyl)-N-(2-methoxy-6-(piperazin-1-yl)pyridin-3-yl)-9,10-dihydro-8H-pyrido[1,6-a:2,3-d′]dipyrimidin-2-amine (Compound 70)

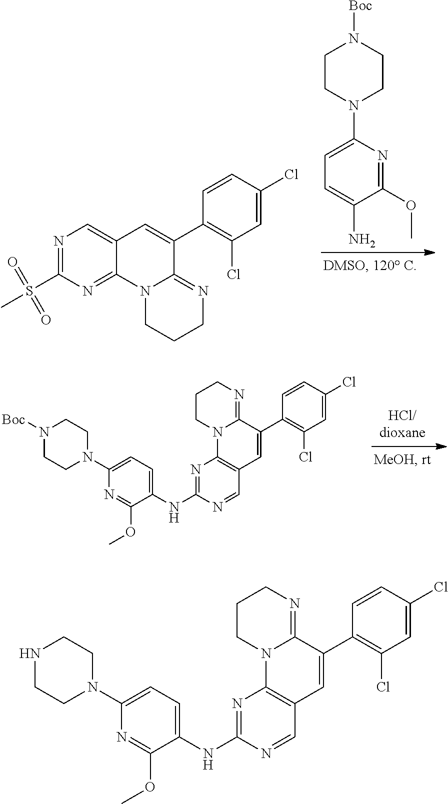

### Step 1: Synthesis of tert-butyl 4-(5-((6-(2,4-dichlorophenyl)-9,10-dihydro-8H-pyrido[1,6-a:2,3-d′]dipyrimidin-2-yl)amino)-6-methoxypyridin-2-yl)piperazine-1-carboxylate

To a solution of 6-(2,4-dichlorophenyl)-2-(methylsulfonyl)-9,10-dihydro-8H-pyrido[1,6-a:2,3-d′]dipyrimidine (170 mg, 0.42 mmol) in DMSO (4 mL) was added tert-butyl 4-(5-amino-6-methoxypyridin-2-yl)piperazine-1-carboxylate (128 mg, 0.42 mmol). The reaction mixture was stirred at 120° C. for 2 hours under N₂. The mixture was added water, extracted with EtOAc (10 mL×3), washed with brine (50 mL), dried over anhydrous Na₂SO₄, concentrated in vacuum and purified by prep-TLC (DCM/MeOH=20:1) to afford tert-butyl 4-(5-((6-(2,4-dichlorophenyl)-9,10-dihydro-8H-pyrido[1,6-a:2,3-d′]dipyrimidin-2-yl)amino)-6-methoxypyridin-2-yl)piperazine-1-carboxylate (100 mg, crude) as a black solid. LCMS (M+H⁺) m/z: 637.3.

### Step 2: Synthesis of 6-(2,4-dichlorophenyl)-N-(2-methoxy-6-(piperazin-1-yl)pyridin-3-yl)-9,10-dihydro-8H-pyrido[1,6-a:2,3-d′]dipyrimidin-2-amine

To a solution of tert-butyl 4-(5-((6-(2,4-dichlorophenyl)-9,10-dihydro-8H-pyrido[1,6-a:2,3-d′]dipyrimidin-2-yl)amino)-6-methoxypyridin-2-yl)piperazine-1-carboxylate (100 mg, crude) in MeOH (2 mL), was added HCl/dioxane (3 mL, 3M). The mixture was stirred at room temperature for 2 hours under N₂. The residue was concentrated and purified by prep-HPLC (0.1% TFA, CH₃CN in H₂O) to give 6-(2,4-dichlorophenyl)-N-(2-methoxy-6-(piperazin-1-yl)pyridin-3-yl)-9,10-dihydro-8H-pyrido[1,6-a:2,3-d′]dipyrimidin-2-amine (3.8 mg, 3% yield, TFA salt) as a yellow solid. ¹H NMR (400 MHz, DMSO-d₆): δ 9.00-8.80 (m, 1H), 8.02 (s, 1H), 7.91-7.87 (m, 1H), 7.64 (dd, J=8.4, 2.0 Hz, 1H), 7.51 (d, J=8.0 Hz, 1H), 7.35 (br, 1H), 6.51 (d, J=8.0 Hz, 1H), 4.38-4.24 (m, 2H), 3.87 (s, 3H), 3.79-3.70 (m, 4H), 3.43-3.37 (m, 2H), 3.28-3.23 (m, 4H), 2.14-2.09 (m, 2H). LCMS (M+H⁺) m/z: 537.2.

### Example 71: Preparation of 1-(3-chloro-4-(2-(methylamino)-9,10-dihydro-8H-pyrido[1,6-a:2,3-d′]dipyrimidin-6-yl)phenyl)-3-methylimidazolidin-2-one (Compound 71)

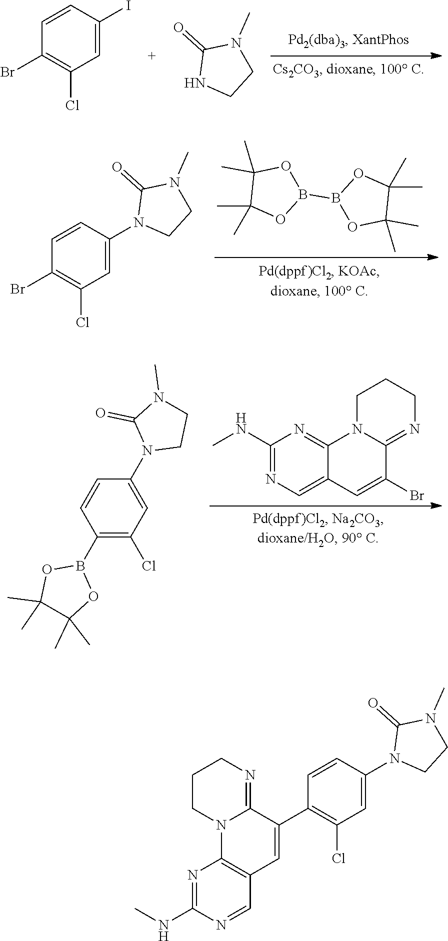

### Step 1: Synthesis of 1-(4-bromo-3-chlorophenyl)-3-methylimidazolidin-2-one

To a solution of 1-methylimidazolidin-2-one (1.0 g, 10.0 mmol) in dioxane (10 mL) were added Pd₂(dba)₃ (91.5 mg, 1 mmol), Cs₂CO₃ (6.5 g, 20 mmol), XantPhos (1.156 g, 2 mmol) and 1-bromo-2-chloro-4-iodobenzene (6.34 g, 20 mmol) at room temperature purged and was degassed with N₂ for 3 times, the mixture was stirred at 100° C. for 4 hours. The mixture was diluted with water (50 mL) and then extracted with EtOAc (50 mL×3). The residue was purified by column chromatography on silica gel (PE/EtOAc=3:1) to afford 1-(4-bromo-3-chlorophenyl)-3-methylimidazolidin-2-one (1.3 g, 86% yield). LCMS (M+H⁺) m/z: 290.6.

### Step 2: Synthesis of 1-(3-chloro-4-(4,4,5,5-tetramethyl-1,3,2-dioxaborolan-2-yl)phenyl)-3-methylimidazolidin-2-one

To a solution of 1-(4-bromo-3-chlorophenyl)-3-methylimidazolidin-2-one (1.0 g, 10.0 mmol) in dioxane (10 mL) was added Pd(dppf)Cl₂ (1.3 g, 4.49 mmol), KOAc (1.32 g, 13.4 mmol) and 4,4,4′,4′,5,5,5′,5′-octamethyl-2,2′-bi(1,3,2-dioxaborolane) (3.4 g, 13.4 mmol) at room temperature purged and was degassed with N₂ for 3 times, the mixture was stirred at 100° C. for 18 hours. The mixture was diluted with water (50 mL) and then extracted with EtOAc (50 mL×3). The residue was purified by column chromatography on silica gel (PE/EtOAc=3:1) to afford 1-(3-chloro-4-(4,4,5,5-tetramethyl-1,3,2-dioxaborolan-2-yl)phenyl)-3-methylimidazolidin-2-one (250 mg, 21% yield). LCMS (M+H⁺) m/z: 337.5

### Step 3: Synthesis of 1-(3-chloro-4-(2-(methylamino)-9,10-dihydro-8H-pyrido[1,6-a:2,3-d′]dipyrimidin-6-yl)phenyl)-3-methylimidazolidin-2-one

To a solution of 1-(3-chloro-4-(4,4,5,5-tetramethyl-1,3,2-dioxaborolan-2-yl)phenyl)-3-methylimidazolidin-2-one (150 mg, 0.44 mmol) in dioxane/H₂O (5 mL) were added Pd(dppf)Cl₂ (32 mg, 0.044 mmol), Na₂CO₃ (140 mg, 1.32 mmol) and 6-bromo-N-methyl-9,10-dihydro-8H-pyrido[1,6-a:2,3-d′]dipyrimidin-2-amine (131 mg, 0.44 mmol) at room temperature purged and was degassed with N₂ for 3 times, the mixture was stirred at 100° C. for 18 hours. The mixture was diluted with water (50 mL) and then extracted with EtOAc (50 mL×3). The reaction mixture was concentrated in vacuum to remove solvent and then purified on prep-HPLC (0.1% HCl) to afford 1-(3-chloro-4-(2-(methylamino)-9,10-dihydro-8H-pyrido[1,6-a:2,3-d′]dipyrimidin-6-yl)phenyl)-3-methylimidazolidin-2-one (15.2 mg, 10% yield, HCl salt) as a white solid. ¹H NMR (400 MHz, CD₃OD): δ 8.82 (s, 1H), 7.99 (d, J=2.0 Hz, 1H), 7.88 (s, 1H), 7.60 (d, J=6.8 Hz, 1H), 7.42 (d, J=8.4 Hz, 1H), 4.70-4.57 (m, 2H), 3.94-3.90 (m, 2H), 3.63-3.56 (m, 4H), 3.10 (s, 3H), 2.90 (s, 3H), 2.38-3.20 (m, 2H). LCMS (M+H⁺) m/z: 424.1.

### Example 72: Preparation of 1-(3-chloro-4-(2-(methylamino)-9,10-dihydro-8H-pyrido[1,6-a:2,3-d′]dipyrimidin-6-yl)phenyl)-3-methylpyridin-2(1H)-one (Compound 72)

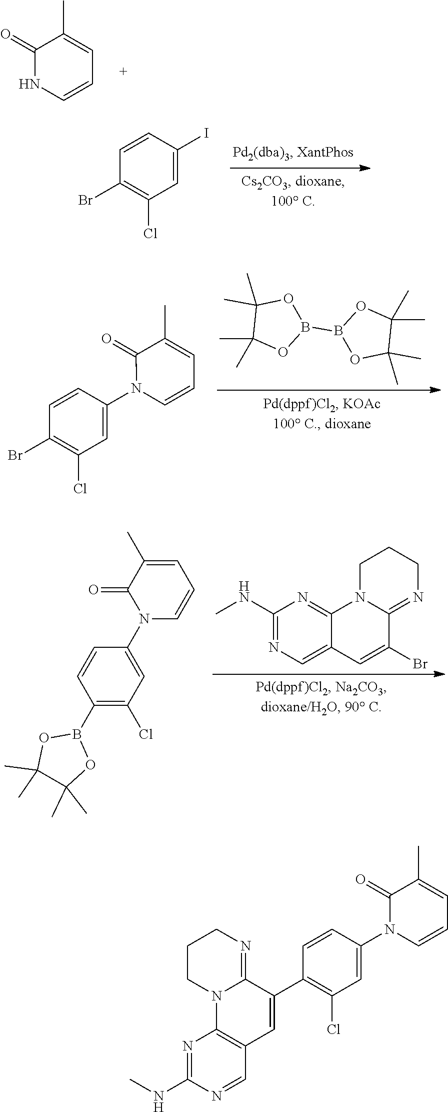

### Step 1: Synthesis of 1-(4-bromo-3-chlorophenyl)-3-methylpyridin-2(1H)-one

To a solution of 3-methylpyridin-2(1H)-one (1.1 g, 10 mmol) in dioxane (10 mL) was added Pd₂(dba)₃ (460 mg, 0.5 mmol), Cs₂CO₃ (3.25 g, 10 mmol), Xant-Phos (578 mg, 1 mmol) and 1-bromo-2-chloro-4-iodobenzene (1.59 g, 5 mmol) at room temperature purged and was degassed with N₂ for 3 times, the mixture was stirred at 100° C. for 4 hours. The mixture was diluted with water (50 mL) and then extracted with EtOAc (50 mL×3). The residue was purified by column chromatography on silica gel (PE/EtOAc=3:1) to afford 1-(4-bromo-3-chlorophenyl)-3-methylpyridin-2(1H)-one (613 mg, 41% yield). LCMS (M+H⁺) m/z: 297.9.

### Step 2: Synthesis of 1-(3-chloro-4-(4,4,5,5-tetramethyl-1,3,2-dioxaborolan-2-yl)phenyl)-3-methylpyridin-2(1H)-one

To a solution of 1-(4-bromo-3-chlorophenyl)-3-methylpyridin-2(1H)-one (613 mg, 2.05 mmol) in dioxane (10 mL) was added Pd(dppf)Cl₂ (150 mg, 0.205 mmol), KOAc (401 mg, 4.1 mmol) and 4,4,4′,4′,5,5,5′,5′-octamethyl-2,2′-bi(1,3,2-dioxaborolane) (1.56 g, 6.15 mmol) at room temperature purged and was degassed with N₂ for 3 times, the mixture was stirred at 100° C. for 18 hours. The mixture was diluted with water (50 mL) and then extracted with EtOAc (50 mL×3). The residue was purified on prep-HPLC to afford 1-(3-chloro-4-(4,4,5,5-tetramethyl-1,3,2-dioxaborolan-2-yl)phenyl)-3-methylpyridin-2(1H)-one (320 mg, 21% yield). LCMS (M+H⁺) m/z: 345.7.

### Step 3: Synthesis of 1-(3-chloro-4-(2-(methylamino)-9,10-dihydro-8H-pyrido[1,6-a:2,3-d′]dipyrimidin-6-yl)phenyl)-3-methylpyridin-2(1H)-one

To a solution of 1-(3-chloro-4-(4,4,5,5-tetramethyl-1,3,2-dioxaborolan-2-yl)phenyl)-3-methylpyridin-2(1H)-one (100 mg, 0.34 mmol) in dioxane/H₂O (5 mL) was added Pd(dppf)Cl₂ (25 mg, 0.034 mmol), Cs₂CO₃ (331 mg, 1.02 mmol) and 6-bromo-N-methyl-9,10-dihydro-8H-pyrido[1,6-a:2,3-d′]dipyrimidin-2-amine (140 mg, 0.40 mmol) at room temperature purged and was degassed with N₂ for 3 times, the mixture was stirred at 100° C. for 18 hours. The mixture was diluted with water (50 mL) and then extracted with EtOAc (50 mL×3). The reaction mixture was concentrated to remove solvent and then purified on prep-HPLC (0.1% TFA, CH₃CN in H₂O) to afford 1-(3-chloro-4-(2-(methylamino)-9,10-dihydro-8H-pyrido[1,6-a:2,3-d′]dipyrimidin-6-yl)phenyl)-3-methylpyridin-2(1H)-one (4.2 mg, 3% yield, TFA salt) as a white solid. ¹H NMR (400 MHz, CD₃OD): δ 8.79 (s, 1H), 7.98 (s, 1H), 7.76 (d, J=1.6 Hz, 1H), 7.66 (d, J=8.0 Hz, 1H), 7.57-7.53 (m, 3H), 6.46 (t, J=6.8 Hz, 1H), 4.78-4.61 (m, 2H), 3.62-3.57 (m, 2H), 3.10 (s, 3H), 2.31-2.26 (m, 2H), 2.18 (s, 3H). LCMS (M+H⁺) m/z: 433.1.

### Example 73: Preparation of 1-(3-chloro-4-(2-(methylamino)-9,10-dihydro-8H-pyrido[1,6-a:2,3-d′]dipyrimidin-6-yl)phenyl)-3-methylpyrazin-2(1H)-one (Compound 73)

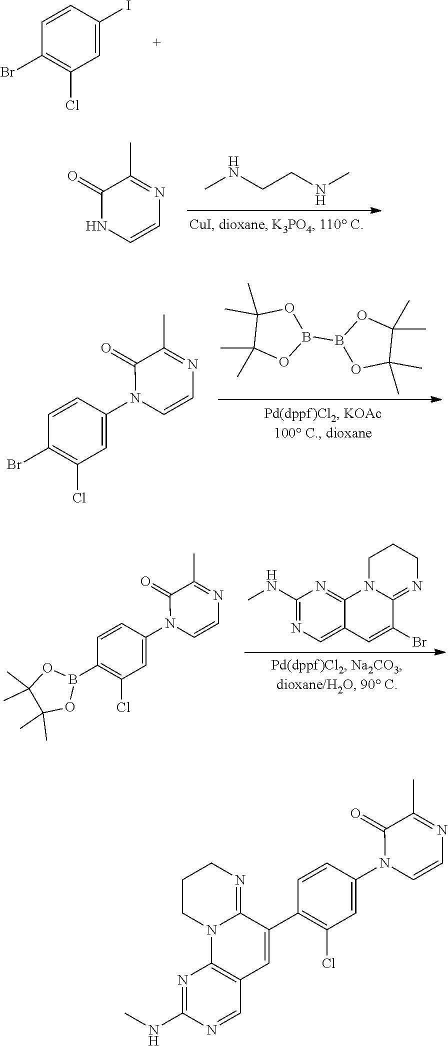

### Step 1: Synthesis of 1-(4-bromo-3-chlorophenyl)-3-methylpyrazin-2(1H)-one

To a solution of 3-methylpyrazin-2(1H)-one (500 mg, 4.54 mmol) in dioxane (15 mL) was added CuI (130 mg, 0.68 mmol), K₃PO₄ (962 mg, 4.54 mmol), N¹,N²-dimethylethane-1,2-diamine (120 mg, 1.36 mmol) and 1-bromo-2-chloro-4-iodobenzene (717 mg, 2.27 mmol) at room temperature purged and was degassed with N₂ for 3 times, the mixture was stirred at 110° C. for 18 hours. The solvent was removed and the residue was purified by column chromatography on silica gel (PE/EtOAc=1:1) to afford 1-(4-bromo-3-chlorophenyl)-3-methylpyrazin-2(1H)-one (220 mg, 32% yield). LCMS (M+H⁺) m/z: 299.0

### Step 2: Synthesis of 1-(3-chloro-4-(4,4,5,5-tetramethyl-1,3,2-dioxaborolan-2-yl)phenyl)-3-methylpyrazin-2(1H)-one

To a solution of 1-(4-bromo-3-chlorophenyl)-3-methylpyrazin-2(1H)-one (220 mg, 0.73 mmol) in dioxane (10 mL) was added Pd(dppf)Cl₂ (150 mg, 0.205 mmol), KOAc (301 mg, 3.1 mmol) and 4,4,4′,4′,5,5,5′,5′-octamethyl-2,2′-bi(1,3,2-dioxaborolane) (360 mg, 1.5 mmol) at room temperature purged and was degassed with N₂ for 3 times, the mixture was stirred at 100° C. for 18 hours. The mixture was diluted with water (50 mL) and then extracted with EtOAc (50 mL×3). The residue was purified on prep-HPLC to afford 1-(3-chloro-4-(4,4,5,5-tetramethyl-1,3,2-dioxaborolan-2-yl)phenyl)-3-methylpyrazin-2(1H)-one (250 mg, 99% yield). LCMS (M+H⁺) m/z: 347.1

### Step 3: Synthesis of 1-(3-chloro-4-(2-(methylamino)-9,10-dihydro-8H-pyrido[1,6-a:2,3-d′]dipyrimidin-6-yl)phenyl)-3-methylpyrazin-2(1H)-one

To a solution of 1-(3-chloro-4-(4,4,5,5-tetramethyl-1,3,2-dioxaborolan-2-yl)phenyl)-3-methylpyrazin-2(1H)-one (71 mg, 0.2 mmol) in dioxane/H₂O (5 mL/1 mL) was added Pd(dppf)Cl₂ (12.4 mg, 0.017 mmol), Na₂CO₃ (36 mg, 0.34 mmol) and 6-bromo-N-methyl-9,10-dihydro-8H-pyrido[1,6-a:2,3-d′]dipyrimidin-2-amine (50 mg, 0.17 mmol) at room temperature purged and was degassed with N₂ for 3 times, the mixture was stirred at 100° C. for 18 hours. The mixture was diluted with water (50 mL) and then extracted with EtOAc (50 mL×3). The reaction mixture was concentrated in vacuum to remove solvent and then purified on prep-HPLC (0.1% TFA, CH₃CN in H₂O) to afford 1-(3-chloro-4-(2-(methylamino)-9,10-dihydro-8H-pyrido[1,6-a:2,3-d′]dipyrimidin-6-yl)phenyl)-3-methylpyrazin-2(1H)-one (2.4 mg, 3% yield, TFA salt) as a yellow solid. ¹H NMR (400 MHz, CD₃OD): δ 8.88-8.78 (m, 1H), 7.97 (s, 1H), 7.85 (s, 1H), 7.69-7.64 (m, 2H), 7.47 (d, J=4.4 Hz, 1H), 7.36 (d, J=4.4 Hz, 1H), 4.76-4.61 (m, 2H), 3.60-3.56 (m, 2H), 3.10-3.08 (m, 3H), 2.47 (s, 3H), 2.30-2.27 (m, 2H). LCMS (M+H⁺) m/z: 434.1.

### Example 74: Preparation of N-(3-chloro-4-(2-(methylamino)-9,10-dihydro-8H-pyrido[1,6-a:2,3-d′]dipyrimidin-6-yl)pyridin-2-yl)propane-1-sulfonamide (Compound 74)

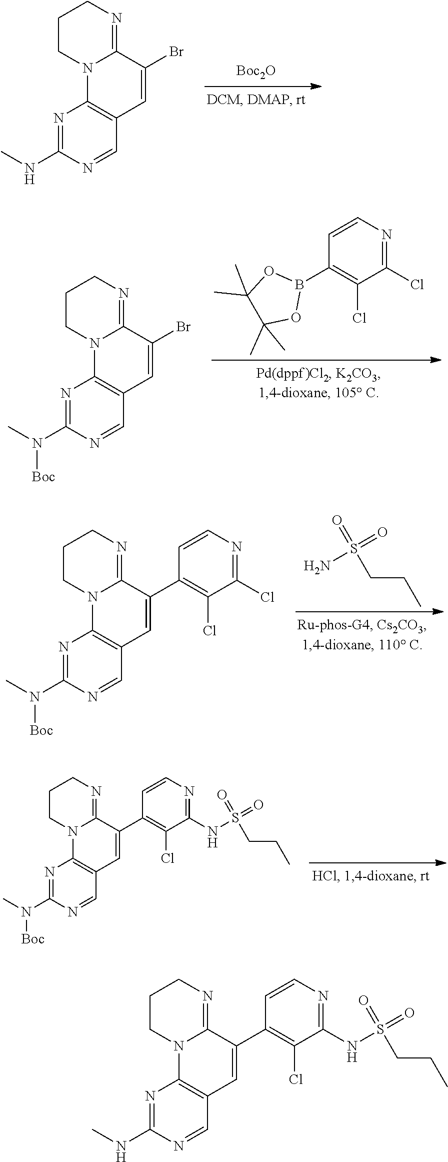

### Step 1. Synthesis of tert-butyl (6-bromo-9,10-dihydro-8H-pyrido[1,6-a:2,3-d′]dipyrimidin-2-yl)(methyl)carbamate

A solution of 6-bromo-N-methyl-9,10-dihydro-8H-pyrido[1,6-a:2,3-d′]dipyrimidin-2-amine (60 mg, 0.2 mmol), Boc₂O (86 mg, 0.4 mmol), DMAP (122 mg, 1.0 mmol) in DCM (5 mL) was stirred at 25° C. for 18 hours. The reaction mixture was purification by flash column chromatography on silica gel (DCM/MeOH=15:1) to give tert-butyl (6-bromo-9,10-dihydro-8H-pyrido[1,6-a:2,3-d′]dipyrimidin-2-yl)(methyl)carbamate (68 mg, 86% yield) as an orange solid. LCMS (M+H⁺) m/z: 394.0.

### Step 2. Synthesis of tert-butyl (6-(2,3-dichloropyridin-4-yl)-9,10-dihydro-8H-pyrido[1,6-a:2,3-d′]dipyrimidin-2-yl)(methyl)carbamate

tert-Butyl (6-bromo-9,10-dihydro-8H-pyrido[1,6-a:2,3-d′]dipyrimidin-2-yl)(methyl)carbamate (68 mg, 0.17 mmol), 2,3-dichloro-4-(4,4,5,5-tetramethyl-1,3,2-dioxaborolan-2-yl)pyridine (56 mg, 0.20 mmol), K₂CO₃ (70 mg, 0.51 mmol) and Pd(dppf)Cl₂ (12 mg, 20 mol %) were suspended with 1,4-dioxane (1 mL) and water (0.1 mL) at protected by N₂. The reaction mixture was refluxed for 3˜5 hours at 105° C. The reaction mixture was cooled to room temperature and filtered, dry loaded onto silica, and purified by flash chromatography on silica gel (DCM/MeOH=15:1) to give tert-butyl (6-(2,3-dichloropyridin-4-yl)-9,10-dihydro-8H-pyrido[1,6-a:2,3-d′]dipyrimidin-2-yl)(methyl)carbamate (46 mg, 58% yield). LCMS (M+H⁺) m/z: 461.0.

### Step 3. Synthesis of tert-butyl (6-(3-chloro-2-(propylsulfonamido)pyridin-4-yl)-9,10-dihydro-8H-pyrido[1,6-a:2,3-d′]dipyrimidin-2-yl)(methyl)carbamate

tert-Butyl (6-(2,3-dichloropyridin-4-yl)-9,10-dihydro-8H-pyrido[1,6-a:2,3-d′]dipyrimidin-2-yl)(methyl)carbamate (46 mg, 0.1 mmol), propane-1-sulfonamide (18 mg, 0.15 mmol), Cs₂CO₃ (97 mg, 0.3 mmol) and Ru-phos-G4 (18 mg, 0.02 mmol) were suspended with 1, 4-dioxane (2 mL) at protected by N₂. The reaction mixture was refluxed for 18 hours. The reaction mixture was purified by flash chromatography on silica gel (DCM/MeOH=10:1) to give tert-butyl (6-(3-chloro-2-(propylsulfonamido)pyridin-4-yl)-9,10-dihydro-8H-pyrido[1,6-a:2,3-d′]dipyrimidin-2-yl)(methyl)carbamate (24 mg, 44% yield). LCMS (M+H⁺) m/z: 548.0.

### Step 4. Synthesis of N-(3-chloro-4-(2-(methylamino)-9,10-dihydro-8H-pyrido[1,6-a:2,3-d′]dipyrimidin-6-yl)pyridin-2-yl)propane-1-sulfonamide

To a solution of tert-butyl (6-(3-chloro-2-(propylsulfonamido)pyridin-4-yl)-9,10-dihydro-8H-pyrido[1,6-a:2,3-d′]dipyrimidin-2-yl)(methyl)carbamate (24 mg, 0.044 mmol) in 1,4-dioxane (1.0 mL) was added 4N HCl in 1,4-dioxane (2 mL) saturated solution. After addition, the reaction mixture was stirred at 25° C. for 1 hour. The reaction mixture was concentrated and the residue was purified by Prep-HPLC (0.1% NH₄HCO₃, CH₃CN in H₂O) to give N-(3-chloro-4-(2-(methylamino)-9,10-dihydro-8H-pyrido[1,6-a:2,3-d′]dipyrimidin-6-yl)pyridin-2-yl)propane-1-sulfonamide (9.2 mg, 46% yield) as a white solid. ¹H NMR (400 MHz, DMSO-d₆): δ 8.66-8.60 (m, 1H), 8.05-8.01 (m, 1H), 8.00-7.70 (m, 1H), 6.64-6.60 (m, 1H), 4.40-4.21 (m, 2H), 3.35-3.22 (m, 4H), 2.92 (s, 3H), 2.10-1.96 (m, 2H), 1.75-1.64 (m, 2H), 1.00-0.92 (m, 3H). LCMS (M+H⁺) m/z: 448.0.

### Example 75: Preparation of N-(3-(2-amino-9,10-dihydro-8H-pyrido[1,6-a:2,3-d′]dipyrimidin-6-yl)-4-methylphenyl)-3-(trifluoromethyl)benzamide (Compound 75)

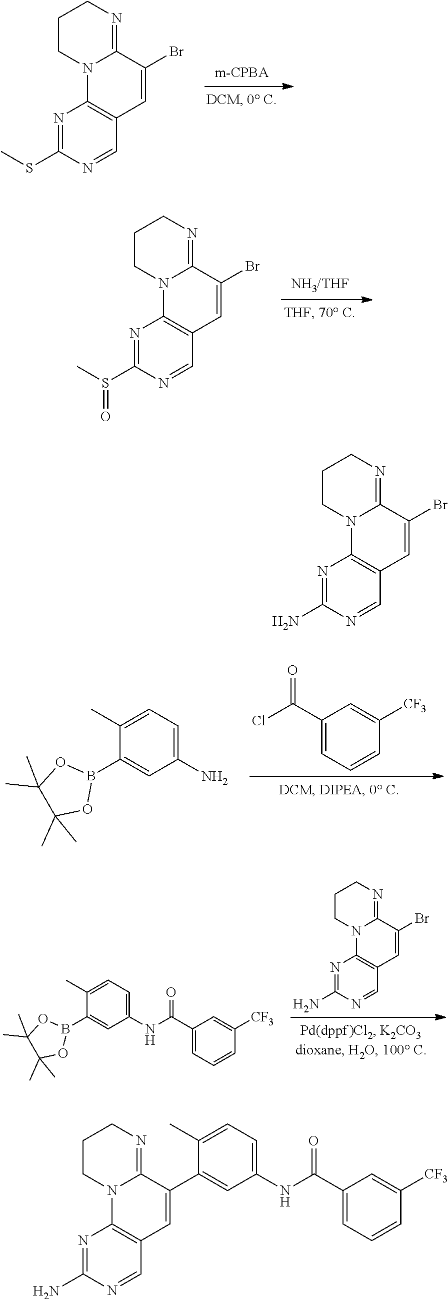

### Step 1: Synthesis of 6-bromo-2-(methylsulfinyl)-9,10-dihydro-8H-pyrido[1,6-a:2,3-d′]dipyrimidine

To a solution of 6-bromo-2-(methylthio)-9,10-dihydro-8H-pyrido[1,6-a:2,3-d′]dipyrimidine (500 mg, 1.6 mmol) in DCM (5 mL) was added m-CPBA (550 mg, 3.2 mmol). The mixture was stirred at room temperature for 1 hour. Concentrated the mixture to afford 6-bromo-2-(methylsulfinyl)-9,10-dihydro-8H-pyrido[1,6-a:2,3-d′]dipyrimidine (501 mg) as a yellow solid, which was to be used into next step without further purification. LCMS (M+H⁺) m/z: 326.9.

### Step 2: Synthesis of 6-bromo-9,10-dihydro-8H-pyrido[1,6-a:2,3-d′]dipyrimidin-2-amine

A mixture of 6-bromo-2-(methylsulfinyl)-9,10-dihydro-8H-pyrido[1,6-a:2,3-d′]dipyrimidine (501 mg, 1.5 mmol) in NH₃/THF (10 mL) was stirred at 70° C. for 16 hours. Concentrated the mixture to give the crude material, which was purified by Prep-HPLC (0.1% NH₄HCO₃, CH₃CN in H₂O) to afford 6-bromo-9,10-dihydro-8H-pyrido[1,6-a:2,3-d′]dipyrimidin-2-amine (130 mg, 31% yield) as a yellow solid. LCMS (M+H⁺) m/z: 280.0.

### Step 3: Synthesis of N-(4-methyl-3-(4,4,5,5-tetramethyl-1,3,2-dioxaborolan-2-yl)phenyl)-3-(trifluoromethyl)benzamide

To a solution of 4-methyl-3-(4,4,5,5-tetramethyl-1,3,2-dioxaborolan-2-yl)aniline (140 mg, 0.5 mmol) in DCM (10 mL) was added 3-(trifluoromethyl)benzoyl chloride (117 mg, 0.5 mmol) and DIPEA (194 mg, 1.5 mmol). The mixture was stirred at 0° C. for 1 hour. Concentrated the mixture to give the crude material. The crude material was purified by column flash chromatography on silica gel (PE/EtOAc=20/1, v/v) to afford N-(4-methyl-3-(4,4,5,5-tetramethyl-1,3,2-dioxaborolan-2-yl)phenyl)-3-(trifluoromethyl)benzamide (124 mg, 61% yield) as a white solid. LCMS (M+H⁺) m/z: 406.0.

### Step 4: Synthesis of N-(3-(2-amino-9,10-dihydro-8H-pyrido[1,6-a:2,3-d′]dipyrimidin-6-yl)-4-methylphenyl)-3-(trifluoromethyl)benzamide

To a solution of 6-bromo-9,10-dihydro-8H-pyrido[1,6-a:2,3-d′]dipyrimidin-2-amine (30 mg, 0.11 mmol) in dioxane (5 mL) was added N-(4-methyl-3-(4,4,5,5-tetramethyl-1,3,2-dioxaborolan-2-yl)phenyl)-3-(trifluoromethyl)benzamide (55 mg, 0.13 mmol), Pd(dppf)Cl₂ (5 mg, 0.006 mmol), K₂CO₃ (46 mg, 0.33 mmol) and H₂O (0.5 mL). The mixture was stirred at 100° C. for 2 hours. Concentrated the mixture to give the crude material. The crude material was purified by prep-HPLC (0.1% Formic acid, CH₃CN in H₂O) to afford N-(3-(2-amino-9,10-dihydro-8H-pyrido[1,6-a:2,3-d′]dipyrimidin-6-yl)-4-methylphenyl)-3-(trifluoromethyl)benzamide (15.5 mg, 30% yield, formic acid salt) as a white solid. ¹HNMR (400 MHz, DMSO-d₆): δ 10.48 (s, 1H), 8.39 (s, 1H), 8.32-8.29 (m, 2H), 8.26 (d, J=8.0 Hz, 1H), 7.97 (d, J=8.0 Hz, 1H), 7.78 (t, J 8.0 Hz, 1H), 7.68 (dd, J 8.0, 2.0 Hz, 1H), 7.60 (d, J=2.0 Hz, 1H), 7.26-7.22 (m, 2H), 7.17 (s, 2H), 4.27 (t, J=5.4 Hz, 2H), 3.42-3.81 (m, 2H), 2.13 (s, 3H), 1.98-1.90 (m, 2H). LCMS (M+H⁺) m/z: 479.0.

### Example 76: Preparation of N-(3-(2-amino-9,10-dihydro-8H-pyrido[1,6-a:2,3-d′]dipyrimidin-6-yl)-4-methylphenyl)-4-(trifluoromethyl)picolinamide (Compound 76)

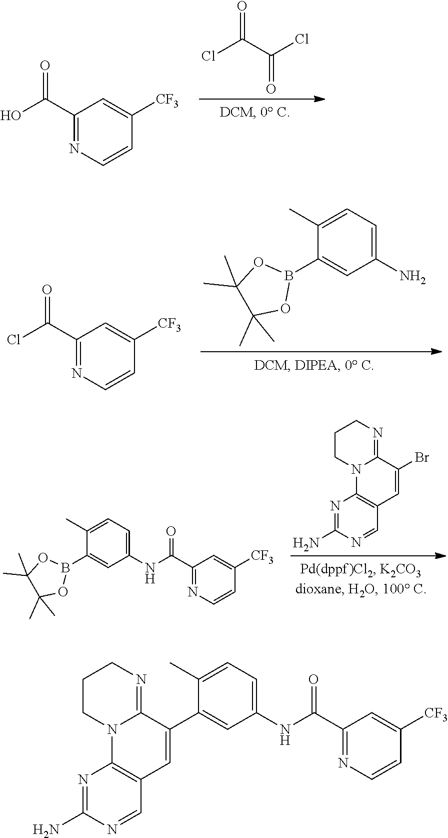

### Step 1: Synthesis of 4-(trifluoromethyl)picolinoyl chloride

To a solution of 4-(trifluoromethyl)picolinic acid (191 mg, 1 mmol) in DCM (5 mL) was added oxalyl dichloride (254 mg, 2 mmol). The mixture was stirred at 0° C. for 1 hour. Concentrated the mixture to afford 4-(trifluoromethyl)picolinoyl chloride (200 mg, crude) as yellow oil.

### Step 2: Synthesis of N-(4-methyl-3-(4,4,5,5-tetramethyl-1,3,2-dioxaborolan-2-yl)phenyl)-4-(trifluoromethyl)picolinamide

To a solution of 4-methyl-3-(4,4,5,5-tetramethyl-1,3,2-dioxaborolan-2-yl)aniline (224 mg, 0.96 mmol) in DCM (10 mL) was added 4-(trifluoromethyl)picolinoyl chloride (200 mg, 0.96 mmol) and DIPEA (370 mg, 2.87 mmol). The mixture was stirred at 0° C. for 1 hour. Concentrated the mixture to give the crude material. The crude material was purified by column flash chromatography on silica gel (PE/EtOAc=20/1, v/v) to afford N-(4-methyl-3-(4,4,5,5-tetramethyl-1,3,2-dioxaborolan-2-yl)phenyl)-4-(trifluoromethyl)picolinamide (160 mg, 41% yield) as a white solid. LCMS (M+H⁺) m/z: 407.0.

### Step 3: Synthesis of N-(3-(2-amino-9,10-dihydro-8H-pyrido[1,6-a:2,3-d′]dipyrimidin-6-yl)-4-methylphenyl)-4-(trifluoromethyl)picolinamide

To a solution of 6-bromo-9,10-dihydro-8H-pyrido[1,6-a:2,3-d′]dipyrimidin-2-amine (30 mg, 0.11 mmol) in dioxane (5 mL) was added N-(4-methyl-3-(4,4,5,5-tetramethyl-1,3,2-dioxaborolan-2-yl)phenyl)-4-(trifluoromethyl)picolinamide (53 mg, 0.13 mmol), Pd(dppf)Cl₂ (5 mg, 0.006 mmol), K₂CO₃ (46 mg, 0.33 mmol) and H₂O (0.5 mL). The mixture was stirred at 100° C. for 2 hours. Concentrated the mixture to give the crude material, which was purified by prep-HPLC (0.1% formic acid, CH₃CN in H₂O) to afford N-(3-(2-amino-9,10-dihydro-8H-pyrido[1,6-a:2,3-d′]dipyrimidin-6-yl)-4-methylphenyl)-4-(trifluoromethyl)picolinamide (11.7 mg, 22% yield, formic acid salt) as a white solid. ¹HNMR (400 MHz, DMSO-d₆): δ 10.76 (s, 1H), 9.03 (d, J=4.8 Hz, 1H), 8.37 (s, 1H), 8.33-8.30 (m, 2H), 8.10-8.08 (m, 1H), 7.78-7.76 (m, 2H), 7.25-7.22 (m, 2H), 7.15 (s, 2H), 4.21-4.16 (m, 2H), 3.42-3.38 (m, 2H), 2.13 (s, 3H), 1.93-1.91 (m, 2H). LCMS (M+H⁺) m/z: 480.0.

### Example 77: Preparation of N-(5-(2-amino-9,10-dihydro-8H-pyrido[1,6-a:2,3-d′]dipyrimidin-6-yl)-6-methylpyridin-3-yl)-3-(trifluoromethyl)benzamide (Compound 77)

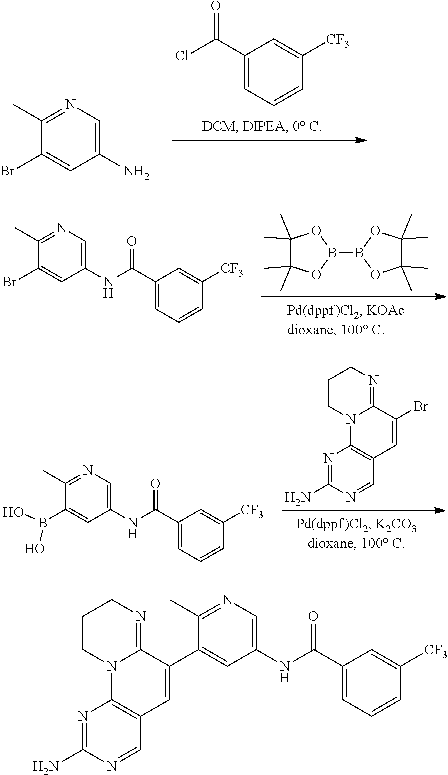

### Step 1: Synthesis of N-(5-bromo-6-methylpyridin-3-yl)-3-(trifluoromethyl)benzamide

To a solution of 5-bromo-6-methylpyridin-3-amine (300 mg, 1.6 mmol) in DCM (10 mL) was added 3-(trifluoromethyl)benzoyl chloride (334 mg, 1.6 mmol) and DIPEA (330 mg, 2.56 mmol). The mixture was stirred at 0° C. for 1 hour. Concentrated the mixture to give the crude material. The crude material was purified by column flash chromatography on silica gel (PE/EtOAc=3:1, v/v) to afford N-(5-bromo-6-methylpyridin-3-yl)-3-(trifluoromethyl)benzamide (331 mg, 58% yield) as a white solid. LCMS (M+H⁺) m/z: 358.9.

### Step 2: Synthesis of (2-methyl-5-(3-(trifluoromethyl)benzamido)pyridin-3-yl)boronic acid

To a solution of N-(5-bromo-6-methylpyridin-3-yl)-3-(trifluoromethyl)benzamide (100 mg, 0.28 mmol) in dioxane (5 mL) was added 4,4,4′,4′,5,5,5′,5′-octamethyl-2,2′-bi(1,3,2-dioxaborolane) (86 mg, 0.34 mmol), Pd(dppf)Cl₂ (10 mg, 0.014 mmol) and KOAc (83 mg, 0.84 mmol). The mixture was stirred at 100° C. for 16 hours. Concentrated the mixture to give the crude product (2-methyl-5-(3-(trifluoromethyl)benzamido)pyridin-3-yl)boronic acid which was used directly in next step. LCMS (M+H⁺) m/z: 325.0.

### Step 3: Synthesis of N-(5-(2-amino-9,10-dihydro-8H-pyrido[1,6-a:2,3-d′]dipyrimidin-6-yl)-6-methylpyridin-3-yl)-3-(trifluoromethyl)benzamide

To a solution of (2-methyl-5-(3-(trifluoromethyl)benzamido)pyridin-3-yl)boronic acid (30 mg, 0.11 mmol) in dioxane (5 mL) was added 6-bromo-9,10-dihydro-8H-pyrido[1,6-a:2,3-d′]dipyrimidin-2-amine (42 mg, 0.13 mmol), Pd(dppf)Cl₂ (5 mg, 0.006 mmol), K₂CO₃ (46 mg, 0.33 mmol) and H₂O (0.5 mL). The mixture was stirred at 100° C. for 2 hours. Concentrated the mixture to give the crude material. The crude material was purified by prep-HPLC (0.1% TFA, CH₃CN in H₂O) to afford N-(5-(2-Amino-9,10-dihydro-8H-pyrido[1,6-a:2,3-d′]dipyrimidin-6-yl)-6-methylpyridin-3-yl)-3-(trifluoromethyl)benzamide (5.6 mg, 11% yield, TFA salt) as a yellow solid. ¹HNMR (400 MHz, DMSO-d₆): δ 10.87 (s, 1H), 8.96 (s, 1H), 8.88-8.85 (m, 2H), 8.33-8.22 (m, 3H), 8.03-7.98 (m, 2H), 7.93-7.81 (m, 3H), 4.49-4.42 (m, 2H), 3.60-3.40 (m, 2H), 2.35 (s, 3H), 2.18-2.14 (m, 2H). LCMS (M+H⁺) m/z: 480.0.

### Example 78: Preparation of N-(5-(2-amino-9,10-dihydro-8H-pyrido[1,6-a:2,3-d′]dipyrimidin-6-yl)-2-methoxypyridin-3-yl)-2,4-difluorobenzenesulfonamide (Compound 78)

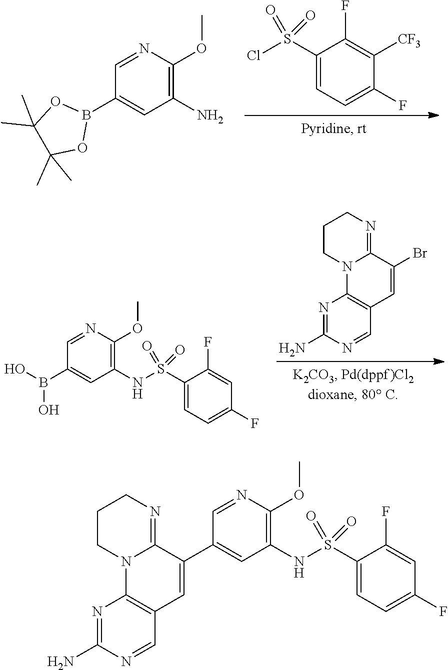

### Step 1: Synthesis of (5-((2,4-difluorophenyl)sulfonamido)-6-methoxypyridin-3-yl)boronic acid

A solution of 2-methoxy-5-(4,4,5,5-tetramethyl-1,3,2-dioxaborolan-2-yl)pyridin-3-amine (100 mg, 0.4 mmol), 2,4-difluorobenzenesulfonyl chloride (102 mg, 0.48 mmol) in pyridine (5 mL) was stirred at room temperature for 16 hours. The mixture was purified by Prep-HPLC (0.1% NH₄HCO₃, CH₃CN in H₂O) to afford (5-((2,4-difluorophenyl)sulfonamido)-6-methoxypyridin-3-yl)boronic acid (60 mg, 44% yield) as a yellow solid.

### Step 2: Synthesis of N-(5-(2-amino-9,10-dihydro-8H-pyrido[1,6-a:2,3-d′]dipyrimidin-6-yl)-2-methoxypyridin-3-yl)-2,4-difluorobenzenesulfonamide

A solution of 6-bromo-9,10-dihydro-8H-pyrido[1,6-a:2,3-d′]dipyrimidin-2-amine (30 mg, 0.11 mmol), (5-((2,4-difluorophenyl)sulfonamido)-6-methoxypyridin-3-yl)boronic acid (48 mg, 0.14 mmol), Pd(dppf)Cl₂ (5 mg) and K₂CO₃ (44 mg, 0.32 mmol) in dioxane (20 mL) was stirred at 80° C. for 16 hours. The mixture was purified by Prep-HPLC (0.1% formic acid, CH₃CN in H₂O) to afford N-(5-(2-amino-9,10-dihydro-8H-pyrido[1,6-a:2,3-d′]dipyrimidin-6-yl)-2-methoxypyridin-3-yl)-2,4-difluorobenzenesulfonamide (9.5 mg, 18% yield, formic acid salt) as a yellow solid. ¹H NMR (400 MHz, DMSO-d₆): δ 8.61 (s, 1H), 8.24 (s, 1H), 7.83-7.75 (m, 1H), 7.55-7.42 (m, 3H), 7.33-7.24 (m, 2H), 7.06 (td, J=8.4, 2.0 Hz, 1H), 4.28-4.26 (m, 2H), 3.75 (s, 3H), 3.35-4.33 (m, 2H), 2.02-2.00 (m, 2H). LCMS (M+H⁺) m/z: 500.1.

### Example 79: Preparation of N-(2-fluorophenyl)-6-(1H-indazol-7-yl)-9,10-dihydro-8H-pyrido[1,6-a:2,3-d′]dipyrimidin-2-amine (Compound 79)

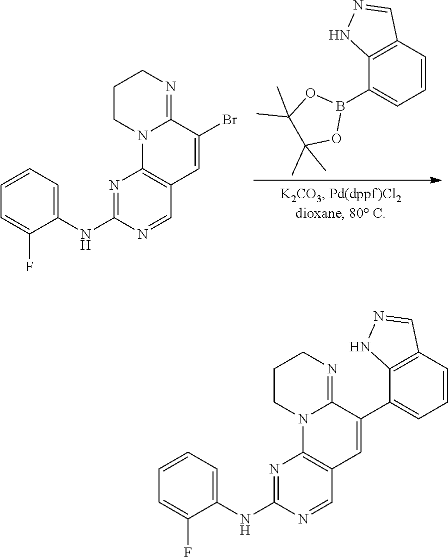

### Step 1: Synthesis of N-(2-fluorophenyl)-6-(1H-indazol-7-yl)-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (40 mg, 0.11 mmol), 7-(4,4,5,5-tetramethyl-1,3,2-dioxaborolan-2-yl)-1H-indazole (40 mg, 0.16 mmol), Pd(dppf)Cl₂ (5 mg) and K₂CO₃ (44 mg, 0.34 mmol) in dioxane (20 mL) was stirred at 80° C. for 16 hours. The mixture was purified by Prep-HPLC (0.1% NH₄HCO₃) twice to give the product, which was not pure enough. Further purification by prep-TLC (DCM/MeOH=2:1) to afford N-(2-fluorophenyl)-6-(1H-indazol-7-yl)-9,10-dihydro-8H-pyrido[1,6-a:2,3-d′]dipyrimidin-2-amine (6.1 mg, 14% yield) as a yellow solid. ¹H NMR (400 MHz, DMSO-d₆): δ 12.67 (s, 1H), 9.33 (s, 1H), 8.41 (s, 1H), 8.06 (s, 1H), 7.87-7.78 (m, 1H), 7.74 (d, J=8.0 Hz, 1H), 7.35-7.10 (m, 6H), 4.12-4.09 (m, 2H), 3.37-3.36 (m, 2H), 1.94-1.90 (m, 2H). LCMS (M+H⁺) m/z: 412.1.

### Example 80: Preparation of 6-(1H-benzo[d]imidazol-4-yl)-N-(2-fluorophenyl)-9,10-dihydro-8H-pyrido[1,6-a:2,3-d′]dipyrimidin-2-amine (Compound 80)

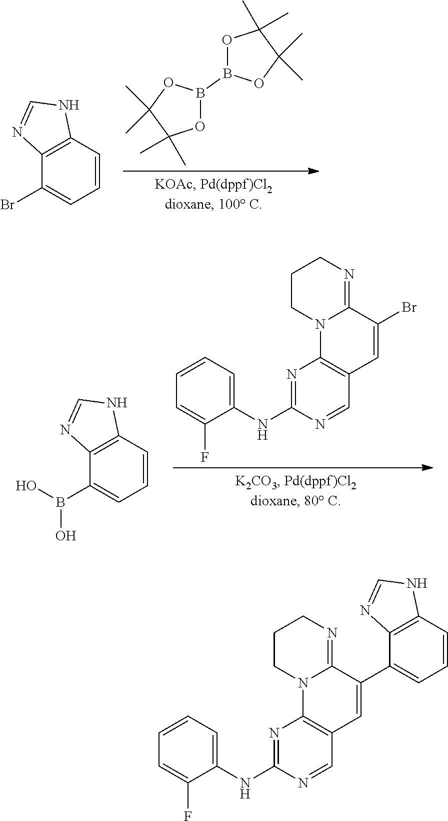

### Step 1: Synthesis of (1H-benzo[d]imidazol-4-yl)boronic acid

A solution of 4-bromo-1H-benzo[d]imidazole (200 mg, 1.02 mmol), 4,4,4′,4′,5,5,5′,5′-octamethyl-2,2′-bi(1,3,2-dioxaborolane) (516 mg, 2.03 mmol), Pd(dppf)Cl₂ (20 mg) and KOAc (200 mg, 2.03 mmol) in dioxane (10 mL) was stirred at 100° C. for 16 hours. The mixture was purified by Prep-HPLC (0.1% NH₄HCO₃, CH₃CN in H₂O) to afford (1H-benzo[d]imidazol-4-yl)boronic acid (30 mg, 18% yield) as a white solid.

### Step 2: Synthesis of 6-(1H-benzo[d]imidazol-4-yl)-N-(2-fluorophenyl)-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (30 mg, 0.08 mmol), (1H-benzo[d]imidazol-4-yl)boronic acid (20 mg, 0.12 mmol), Pd(dppf)Cl₂ (5 mg) and K₂CO₃ (33 mg, 0.24 mmol) in dioxane (2 mL) was stirred at 80° C. for 5 hours. The mixture was purified by Prep-HPLC (0.1% NH₄HCO₃) and prep-TLC (DCM/MeOH=3:1) to afford 6-(1H-benzo[d]imidazol-4-yl)-N-(2-fluorophenyl)-9,10-dihydro-8H-pyrido[1,6-a:2,3-d′]dipyrimidin-2-amine (1.8 mg, 5% yield) as a yellow solid. ¹H NMR (400 MHz, DMSO-d₆): δ 11.98 (br s, 1H), 9.24 (s, 1H), 8.36 (s, 1H), 8.14 (s, 1H), 7.84-7.81 (m, 1H), 7.62-7.60 (m, 1H), 7.32-7.11 (m, 6H), 4.11-4.08 (m, 2H), 3.34-3.26 (m, 2H), 1.89-1.85 (m, 2H). LCMS (M+H⁺) m/z: 412.1.

### Example 81: Preparation of N-(2-fluorophenyl)-6-(1H-pyrazolo[3,4-b]pyridin-4-yl)-9,10-dihydro-8H-pyrido[1,6-a:2,3-d′]dipyrimidin-2-amine (Compound 81)

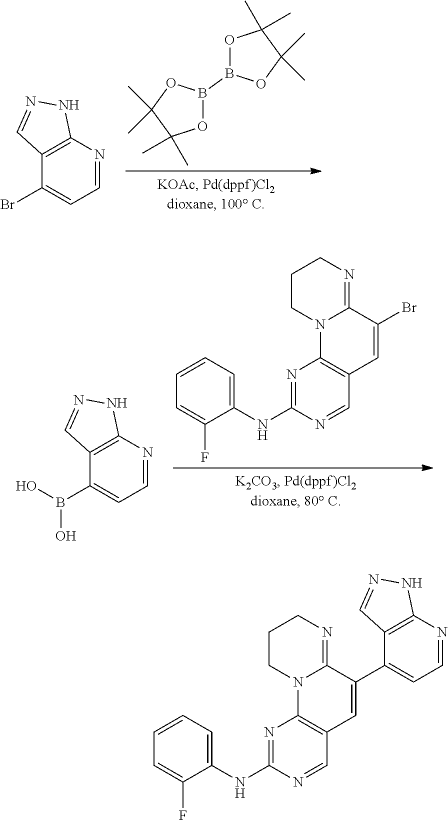

### Step 1: Synthesis of (1H-Pyrazolo[3,4-b]pyridin-4-yl)boronic acid

A solution of 4-bromo-1H-pyrazolo[3,4-b]pyridine (200 mg, 1.01 mmol), 4,4,4′,4′,5,5,5′,5′-octamethyl-2,2′-bi(1,3,2-dioxaborolane) (1282 mg, 5.05 mmol), Pd(dppf)Cl₂ (100 mg) and KOAc (595 mg, 6.06 mmol) in dioxane (10 mL) was stirred at 100° C. for 16 hours. The mixture was purified by Prep-HPLC (0.1% NH₄HCO₃, CH₃CN in H₂O) to afford (1H-pyrazolo[3,4-b]pyridin-4-yl)boronic acid (30 mg, 18% yield) as a white solid.

### Step 2: Synthesis of N-(2-fluorophenyl)-6-(1H-pyrazolo[3,4-b]pyridin-4-yl)-9,10-dihydro-8H-pyrido[1,6-a:2,3-d′]dipyrimidin-2-amine

A solution of 6-bromo-N-(2-fluorophenyl)-9,10-dihydro-8H-pyrido[1,6-a:2,3-d′]dipyrimidin-2-amine (30 mg, 0.08 mmol), (1H-pyrazolo[3,4-b]pyridin-4-yl)boronic acid (20 mg, 0.12 mmol), Pd(dppf)Cl₂ (5 mg) and K₂CO₃ (33 mg, 0.24 mmol) in dioxane (2 mL) was stirred at 80° C. for 5 hours. The mixture was purified by Prep-HPLC (0.1% NH₄HCO₃, CH₃CN in H₂O) to give product, but its purity is not pure enough. Further purification by Pre-TLC (DCM/MeOH=3:1) to afford N-(2-fluorophenyl)-6-(1H-pyrazolo[3,4-b]pyridin-4-yl)-9,10-dihydro-8H-pyrido[1,6-a:2,3-d′]dipyrimidin-2-amine (0.8 mg, 3% yield) as a yellow solid. ¹H NMR (400 MHz, DMSO-d₆): δ 13.54 (br, 1H), 9.35 (s, 1H), 8.48 (d, J=4.8 Hz, 1H), 8.41 (s, 1H), 8.05 (s, 1H), 7.82-7.77 (m, 1H), 7.44 (s, 1H), 7.29-7.16 (m, 4H), 4.07-4.04 (m, 2H), 3.33-3.30 (m, 2H), 1.90-1.88 (m, 2H). LCMS (M+H⁺) m/z: 413.1.

### Example 82: Preparation of (4-chloro-3-((6-(2-chlorophenyl)-8,9-dihydroimidazo[1′,2′:1,6]pyrido[2,3-d]pyrimidin-2-yl)amino)phenyl)methanol (Compound 82)

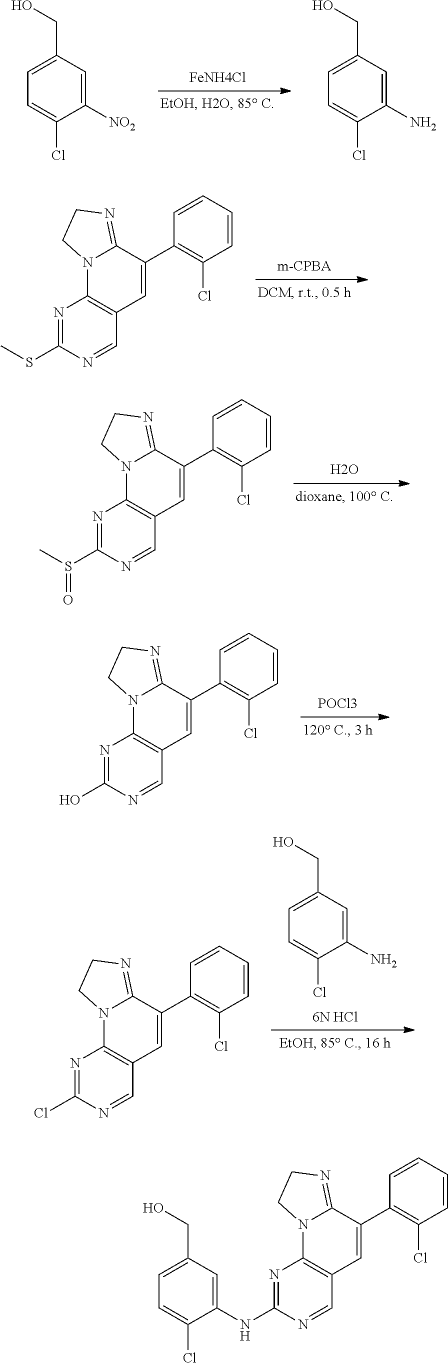

### Step 1: Synthesis of 6-(2-chlorophenyl)-2-(methylsulfinyl)-8,9-dihydroimidazo[1′,2′:1,6]pyrido[2,3-d]pyrimidine

The mixture of 6-(2-chlorophenyl)-2-(methylthio)-8,9-dihydroimidazo[1′,2′:1,6]pyrido[2,3-d]pyrimidine (300 mg, 0.91 mmol, 1.0 eq), m-CPBA (394 mg, 2.28 mmol, 2.5 eq) in DCM (50 mL) was stirred at 25° C. for 0.2 h, The reaction was monitored by LCMS. The mixture was concentrated in vacuum to give crude product (600 mg) as a yellow solid. The crude 6-(2-chlorophenyl)-2-(methylsulfinyl)-8,9-dihydroimidazo[1′,2′:1,6]pyrido[2,3-d]pyrimidine was used next step. LCMS (M+H⁺) m/z: 345.0.

### Step 2: Synthesis of 6-(2-chlorophenyl)-8,9-dihydroimidazo[1′,2′:1,6]pyrido[2,3-d]pyrimidin-2-ol

The mixture of 6-(2-chlorophenyl)-2-(methylsulfinyl)-8,9-dihydroimidazo[1′,2′:1,6]pyrido[2,3-d]pyrimidine (600 mg, 1.74 mmol, 1.0 eq) in dioxane (10 mL) and H₂O (1 mL) was stirred at 100° C. for 1 h. The reaction was monitored by LCMS. The mixture was concentrated in vacuum to give crude product, which was purified on silica gel column flash chromatography (DCM:MeOH=10:1, v/v) to afford 6-(2-chlorophenyl)-8,9-dihydroimidazo[1′,2′:1,6]pyrido[2,3-d]pyrimidin-2-ol (120 mg) as yellow solid. LCMS (M+H⁺) m/z: 298.9.

### Step 3: Synthesis of 2-chloro-6-(2-chlorophenyl)-8,9-dihydroimidazo[1′,2′:1,6]pyrido[2,3-d]pyrimidine

The mixture of 6-(2-chlorophenyl)-8,9-dihydroimidazo[1′,2′:1,6]pyrido[2,3-d]pyrimidin-2-ol (120 mg, 0.4 mmol, 1.0 eq) in POCl₃ (15 mL) was stirred at 120° C. for 5 h. The reaction was monitored by LCMS. The mixture was concentrated in vacuum to give crude product, The crude product was partitioned between EA (50 mL) and H₂O (30 mL), aqueous phase was extracted by EA (50 mL) twice. The organic phase was washed with brine (50 mL), dried Na₂SO₄ and concentrated in vacuum to afford 2-chloro-6-(2-chlorophenyl)-8,9-dihydroimidazo[1′,2′:1,6]pyrido[2,3-d]pyrimidine (120 mg) as a yellow solid. LCMS (M+H⁺) m/z: 317.1.

### Step 4: Synthesis of (3-amino-4-chlorophenyl)methanol

The mixture of (4-chloro-3-nitrophenyl)methanol (1.0 g, 5.33 mmol, 1.0 eq), Fe powder (1.2 g, 21.32 mmol, 4.0 eq) and NH₄Cl (1.7 g, 31.99 mmol, 6.0 eq) in EtOH (30 mL) and H₂O (10 mL) was stirred at 85° C. for 2 h, The reaction was monitored by LCMS. The mixture was filtered and concentrated in vacuum to give crude product. H₂O (10 mL) was added and the precipitate was filtered to afford product (3-amino-4-chlorophenyl)methanol (500 mg) as white solid. LCMS (M+H⁺) m/z: 158.1.

### Step 5: Synthesis of (4-chloro-3-((6-(2-chlorophenyl)-8,9-dihydroimidazo[1′,2′:1,6]pyrido[2,3-d]pyrimidin-2-yl)amino)phenyl)methanol

The mixture of 2-chloro-6-(2-chlorophenyl)-8,9-dihydroimidazo[1′,2′:1,6]pyrido[2,3-d]pyrimidine (50 mg, 0.09 mmol, 1.0 eq), (3-amino-4-chlorophenyl)methanol (75 mg, 0.45 mmol, 3.0 eq) and HCl (6N, 0.3 mL) in EtOH (10 mL) was stirred at 85° C. for 2 h, The reaction was monitored by LCMS. The mixture was purified by Prep-HPLC (0.1% FA) to afford (4-chloro-3-((6-(2-chlorophenyl)-8,9-dihydroimidazo[1′,2′:1,6]pyrido[2,3-d]pyrimidin-2-yl)amino)phenyl)methanol (55.7 mg) as a yellow solid. ¹H NMR (400 MHz, DMSO-d₆): δ 10.29 (s, 1H), 10.15 (s, 1H), 9.01 (s, 1H), 8.27 (s, 1H), 7.69 (dd, J=8.0, 1.2 Hz, 1H), 7.62-7.50 (m, 5H), 7.25 (dd, J=8.0, 1.2 Hz, 1H), 5.38-5.35 (m, 1H), 4.60-4.48 (m, 4H), 4.06-4.01 (m, 2H). LCMS (M+H⁺) m/z: 438.0.

### Example 83: Preparation of (3-((6-(2-chlorophenyl)-8,9-dihydroimidazo[1′,2′:1,6]pyrido[2,3-d]pyrimidin-2-yl)amino)-4-fluorophenyl)methanol (Compound 83)

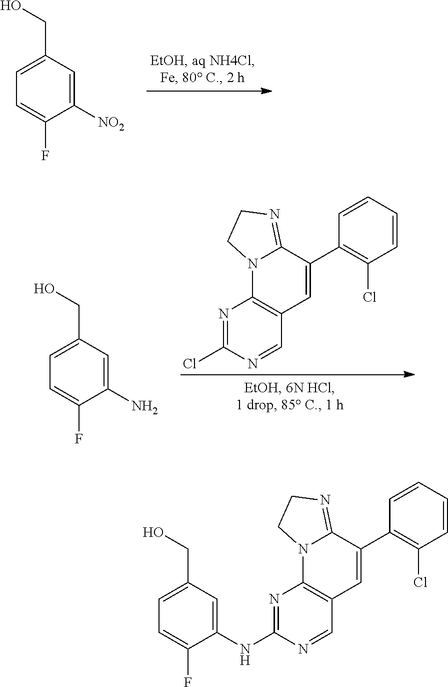 [SMILES: O=[N+]([O-])c1cc(CO)ccc1F ; "EtOH, aq NH4Cl,Fe, 80° C., 2 h" ; Nc1cc(CO)ccc1F ; Clc1ncc2c(n1)N1CCN=C1C(c1ccccc1Cl)=C2 ; OCc1ccc(F)c(Nc2ncc3c(n2)N2CCN=C2C(c2ccccc2Cl)=C3)c1]

### Step 1: Synthesis of (3-amino-4-fluorophenyl)methanol

To a solution of (4-fluoro-3-nitrophenyl)methanol (400 mg, 2.3 mmol) in EtOH (10 mL) was added Fe (258 mg, 4.6 mmol) and NH₄Cl (aq, 369 mg, 6.9 mmol). The mixture was stirred at 80° C. for 2 h. LCMS showed the reaction was complete. The mixture was concentrated and extracted with EA (20 mL×3). The organic layers were concentrated to give the (3-amino-4-fluorophenyl)methanol (310 mg, 95.5% yield) as dark a green solid.

### Step 2: Synthesis of (3-((6-(2-chlorophenyl)-8,9-dihydroimidazo[1′,2′:1,6]pyrido[2,3-d]pyrimidin-2-yl)amino)-4-fluorophenyl)methanol

A mixture of (3-amino-4-fluorophenyl)methanol (18 mg, 0.126 mmol) in EtOH (3 mL) was added 2-chloro-6-(2-chlorophenyl)-8,9-dihydroimidazo[1′,2′:1,6]pyrido[2,3-d]pyrimidine (40 mg, 0.126 mmol) and HCl (6 mol/L, 1 drop). The mixture was stirred at 85° C. for 1 h. The mixture was concentrated and purified by prep-HPLC to give the (3-((6-(2-chlorophenyl)-8,9-dihydroimidazo[1′,2′:1,6]pyrido[2,3-d]pyrimidin-2-yl)amino)-4-fluorophenyl)methanol (27.1 mg, 51% yield) as a yellow solid. ¹H NMR (400 MHz, DMSO-d₆): δ 9.32 (s, 1H), 8.32 (s, 1H), 7.78 (d, J=7.2 Hz, 1H), 7.54-7.51 (m, 1H), 7.44-7.37 (m, 3H), 7.27 (s, 1H), 7.20-7.15 (m, 1H), 7.09-7.07 (m, 1H), 5.24 (br, 1H), 4.48 (s, 2H), 4.02 (t, J=8.8 Hz, 2H), 3.91 (t, J=8.8 Hz, 2H). LCMS (M+H⁺) m/z: 421.7.

### Example 84: Preparation of N-(2-chloro-4-(piperazin-1-yl)phenyl)-6-(2-chlorophenyl)-8,9-dihydroimidazo[1′,2′:1,6]pyrido[2,3-d]pyrimidin-2-amine (Compound 84)

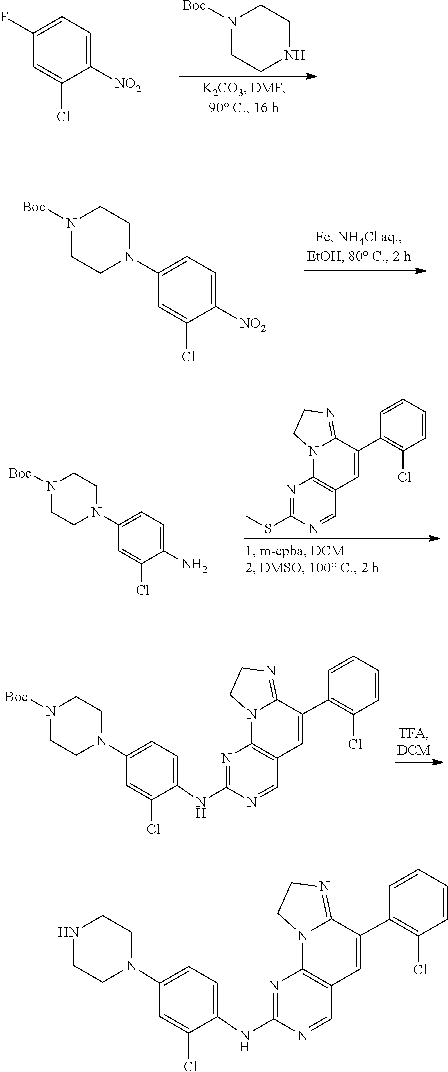

### Step 1: Synthesis of tert-butyl 4-(3-chloro-4-nitrophenyl)piperazine-1-carboxylate

The mixture of 2-chloro-4-fluoro-1-nitrobenzene (1.75 g, 10 mmol), tert-butyl piperazine-1-carboxylate (2.42 mg, 15 mmol) and K₂CO₃ (4.14 g, 30 mmol) were into DMF (20 mL). The reaction was stirred for 16 hours at 100° C. The reaction was detected by LCMS, the reaction was worked complete. The reaction was purification by flash (PE:EA=3:1) to give tert-butyl 4-(3-chloro-4-nitrophenyl)piperazine-1-carboxylate (1.26 g) as white solid. LCMS (M+H⁺) m/z: 342.0.

### Step 2: Synthesis of tert-butyl 4-(4-amino-3-chlorophenyl)piperazine-1-carboxylate

The mixture of tert-butyl 4-(3-chloro-4-nitrophenyl)piperazine-1-carboxylate (680 mg, 2 mmol), Fe (330 mg, 6.0 mmol) were into NH₄Cl aq (3.0 mL) and EtOH (6 mL). The reaction was stirred for 1 hour at 80° C. LCMS showed the reaction was complete. The reaction was filtered and extracted by EA (50 mL) twice. The combined organic phase was dried over with Na₂SO₄, and concentrated to give product tert-butyl 4-(4-amino-3-chlorophenyl)piperazine-1-carboxylate (520 mg) as gray solid. LCMS (M+H⁺) m/z: 312.0.

### Step 3: Synthesis of tert-butyl 4-(3-chloro-4-((6-(2-chlorophenyl)-8,9-dihydroimidazo[1′,2′:1,6]pyrido[2,3-d]pyrimidin-2-yl)amino)phenyl)piperazine-1-carboxylate

The mixture of 6-(2-chlorophenyl)-2-(methylthio)-8,9-dihydroimidazo[1′,2′:1,6]pyrido[2,3-d]pyrimidine (100 mg, 0.3 mmol) and m-CPBA (130 mg, 0.75 mmol) was in DCM (3 mL), and then the mixture was stirred for 0.5 h at 25° C. The reaction was concentrated and a solution of tert-butyl 4-(4-amino-3-chlorophenyl)piperazine-1-carboxylate (466 mg, 1.5 mmol) in DMSO (0.5 mL) was added to reaction mixture. The reaction was stirred at 100° C. for 2 h. The reaction was diluted with water and extracted with EA. The combined organic phase was concentrated and the residue was purified by flash (DCM:MeOH=10:1) to give tert-butyl 4-(3-chloro-4-((6-(2-chlorophenyl)-8,9-dihydroimidazo[1′,2′:1,6]pyrido[2,3-d]pyrimidin-2-yl)amino)phenyl)piperazine-1-carboxylate (74 mg) as gray solid. LCMS (M+H⁺) m/z: 591.9.

### Step 4: Synthesis of N-(2-chloro-4-(piperazin-1-yl)phenyl)-6-(2-chlorophenyl)-8,9-dihydroimidazo[1′,2′:1,6]pyrido[2,3-d]pyrimidin-2-amine

To a solution of tert-butyl 4-(3-chloro-4-((6-(2-chlorophenyl)-8,9-dihydroimidazo[1′,2′:1,6]pyrido[2,3-d]pyrimidin-2-yl)amino)phenyl)piperazine-1-carboxylate (74 mg, 1.25 mmol) in DCM (4.0 mL), was added TFA (2.0 mL). The reaction was stirred for 0.5 h at 25° C. The reaction mixture was concentrated and the residue was purified by HPLC (0.5% TFA) to give N-(2-chloro-4-(piperazin-1-yl)phenyl)-6-(2-chlorophenyl)-8,9-dihydroimidazo[1′,2′:1,6]pyrido[2,3-d]pyrimidin-2-amine (52 mg) as yellow solid. ¹H NMR (400 MHz, DMSO-d₆): δ 10.14 (s, 2H), 8.94-8.90 (m, 3H), 8.25 (s, 1H), 7.69 (dd, J=8.0, 1.2 Hz, 1H), 7.60-7.49 (m, 4H), 7.17 (d, J=2.8 Hz, 1H), 7.03 (dd, J=9.2, 2.8 Hz, 1H), 4.50 (br, 2H), 3.95-3.90 (m, 2H), 3.42-3.36 (m, 4H), 3.25-3.24 (m, 4H). LCMS (M+H⁺) m/z: 492.0.

### Example 85: Preparation of N-(2-chloro-4-(4-methylpiperazin-1-yl)phenyl)-6-(2-chlorophenyl)-8,9-dihydroimidazo[1′,2′:1,6]pyrido[2,3-d]pyrimidin-2-amine (Compound 85)

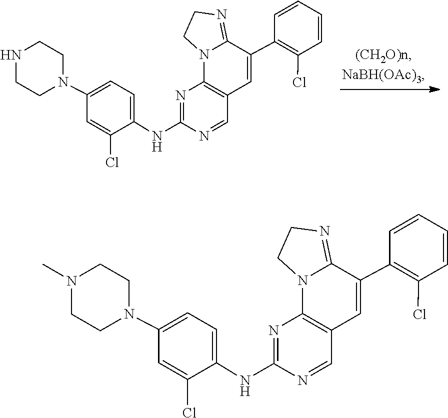 [SMILES: Clc1cc(N2CCNCC2)ccc1Nc1ncc2c(n1)N1CCN=C1C(c1ccccc1Cl)=C2 ; CN1CCN(c2ccc(Nc3ncc4c(n3)N3CCN=C3C(c3ccccc3Cl)=C4)c(Cl)c2)CC1]

### Step 1: Synthesis of N-(2-chloro-4-(4-methylpiperazin-1-yl)phenyl)-6-(2-chlorophenyl)-8,9-dihydroimidazo[1′,2′:1,6]pyrido[2,3-d]pyrimidin-2-amine

The mixture of N-(2-chloro-4-(piperazin-1-yl)phenyl)-6-(2-chlorophenyl)-8,9-dihydroimidazo[1′,2′:1,6]pyrido[2,3-d]pyrimidin-2-amine (25 mg, 0.05 mmol) was into THE (4.0 mL), then (CH₂O)n (13 mg) and NaBH(OAc)3 (32 mg, 0.015 mmol) were added to reaction mixture. The reaction was stirred for 5 hours at 25° C. The reaction was detected by LCMS, the reaction was complete. The reaction was poured in to water (10 mL), adjust to pH=11 with aq NaHCO₃, extracted with EA (15 mL×2). The combined organic phase was dried over with Na₂SO₄, concentrated and purified by HPLC to give product N-(2-chloro-4-(4-methylpiperazin-1-yl)phenyl)-6-(2-chlorophenyl)-8,9-dihydroimidazo[1′,2′:1,6]pyrido[2,3-d]pyrimidin-2-amine (13.5 mg) as yellow solid. ¹H NMR (400 MHz, DMSO-d₆): δ 9.00 (s, 1H), 8.28 (s, 1H), 7.54-7.53 (m, 1H), 7.51-7.37 (m, 4H), 7.28 (s, 1H), 7.01 (d, J=2.8 Hz, 1H), 6.92 (dd, J=8.8, 2.8 Hz, 1H), 4.01-3.99 (m, 2H), 3.91-3.89 (m, 2H), 3.17-3.15 (m, 4H), 2.50-2.49 (m, 4H), 2.24 (s, 3H). LCMS (M+H⁺) m/z: 506.0.

### Example 86: Preparation of N-(2-chloro-4-morpholinophenyl)-6-(2-chlorophenyl)-8,9-dihydroimidazo[1′,2′:1,6]pyrido[2,3-d]pyrimidin-2-amine (Compound 86)

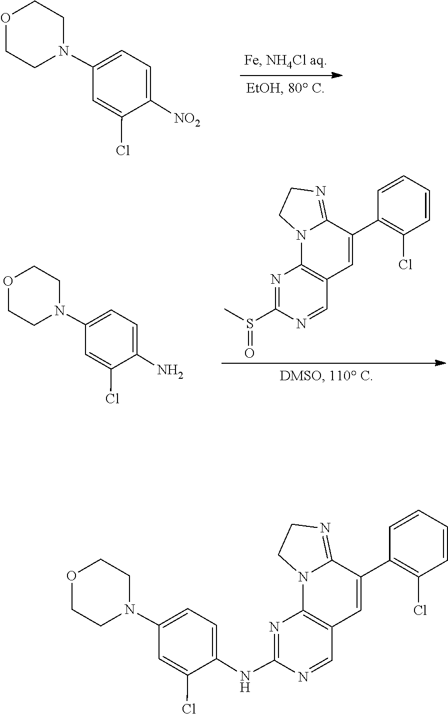 [SMILES: O=[N+]([O-])c1ccc(N2CCOCC2)cc1Cl ; Nc1ccc(N2CCOCC2)cc1Cl ; CS(=O)c1ncc2c(n1)N1CCN=C1C(c1ccccc1Cl)=C2 ; Clc1cc(N2CCOCC2)ccc1Nc1ncc2c(n1)N1CCN=C1C(c1ccccc1Cl)=C2]

### Step 1: Synthesis of 2-chloro-4-morpholinoaniline

To a solution of 4-(3-chloro-4-nitrophenyl)morpholine (363 mg, 1.5 mmol) in EtOH (5 mL) was added Fe powder (168 mg, 3.0 mmol) and saturated NH₄Cl a.q. (1 mL), the reaction mixture was stirred at 80° C. for 2 h. LCMS showed the reaction completed. The reaction mixture was filtered by celite, concentrated to obtain a yellow solid, which was purified by chromatography column (DCM:MeOH=10:1) to afford 2-chloro-4-morpholinoaniline (360 mg, 85% yield) as a gray solid. LCMS (M+H⁺) m/z: 213.1.

### Step 2: Synthesis of N-(2-chloro-4-morpholinophenyl)-6-(2-chlorophenyl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 2-chloro-4-morpholinoaniline (360 mg, 1.7 mmol). The mixture was stirred at 110° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by Prep-HPLC to afford N-(2-chloro-4-morpholinophenyl)-6-(2-chlorophenyl)-8,9-dihydroimidazo[1′,2′:1,6]pyrido[2,3-d]pyrimidin-2-amine (9.5 mg) as yellow solid. ¹H NMR (400 MHz, DMSO-d₆): δ 8.99 (br, 1H), 8.28 (br, 1H), 7.53-7.46 (m, 2H), 7.43-7.37 (m, 3H), 7.27 (br, 1H), 7.03 (d, J=2.4 Hz, 1H), 6.93 (dd, J=8.8, 2.4 Hz, 1H), 4.01-3.96 (m, 2H), 3.92-3.87 (m, 2H), 3.75-3.72 (m, 4H), 3.15-3.10 (m, 4H). LCMS (M+H⁺) m/z: 493.0.

### Example 87: Preparation of 6-(2-chlorophenyl)-N-(4-(piperazin-1-yl)phenyl)-8,9-dihydroimidazo[1′,2′:1,6]pyrido[2,3-d]pyrimidin-2-amine (Compound 87)

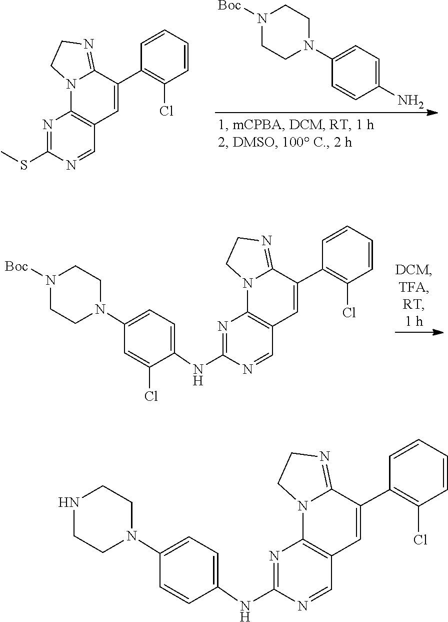 [SMILES: CSc1ncc2c(n1)N1CCN=C1C(c1ccccc1Cl)=C2 ; CC(C)(C)OC(=O)N1CCN(c2ccc(N)cc2)CC1 ; CC(C)(C)OC(=O)N1CCN(c2ccc(Nc3ncc4c(n3)N3CCN=C3C(c3ccccc3Cl)=C4)c(Cl)c2)CC1 ; Clc1ccccc1C1=Cc2cnc(Nc3ccc(N4CCNCC4)cc3)nc2N2CCN=C12]

### Step 1: Synthesis of tert-butyl 4-(4-((6-(2-chlorophenyl)-8,9-dihydroimidazo[1′,2′:1,6]pyrido[2,3-d]pyrimidin-2-yl)amino)phenyl)piperazine-1-carboxylate

To a mixture of 6-(2-chlorophenyl)-2-(methylthio)-8,9-dihydroimidazo[1′,2′:1,6]pyrido[2,3-d]pyrimidine (100 mg, 0.30 mmol) in DCM (2 mL) was added m-CPBA (155 mg, 0.90 mmol) at room temperature and stirred for 20 min. The mixture was concentrated and added tert-butyl 4-(4-aminophenyl)piperazine-1-carboxylate (416 mg, 1.5 mmol) and DMSO (0.2 mL). The reaction mixture was stirred at 100° C. for 2 h. The reaction mixture was purified by column chromatography (DCM:MeOH=13:1) to give tert-butyl 4-(4-((6-(2-chlorophenyl)-8,9-dihydroimidazo[1′,2′:1,6]pyrido[2,3-d]pyrimidin-2-yl)amino)phenyl)piperazine-1-carboxylate (120 mg, 71.7% yield) as dark green oil. LCMS (M+H⁺) m/z: 591.8.

### Step 2: Synthesis of 6-(2-chlorophenyl)-N-(4-(piperazin-1-yl)phenyl)-8,9-dihydroimidazo[1′,2′:1,6]pyrido[2,3-d]pyrimidin-2-amine

To a solution of tert-butyl 4-(4-((6-(2-chlorophenyl)-8,9-dihydroimidazo[1′,2′:1,6]pyrido[2,3-d]pyrimidin-2-yl)amino)phenyl)piperazine-1-carboxylate (120 mg, 0.22 mmol) in DCM (6 mL) was added TFA (3 mL). The mixture was stirred at room temperature for 3 h. LCMS and TLC monitored the reaction. Concentration gave the crude material and the pH was adjusted to 7. The crude material was purified by prep-HPLC to afford 6-(2-chlorophenyl)-N-(4-(piperazin-1-yl)phenyl)-8,9-dihydroimidazo[1′,2′:1,6]pyrido[2,3-d]pyrimidin-2-amine (55.4 mg, 55.0% yield) as a brown solid. ¹H NMR (400 MHz, DMSO-d₆): δ 10.67 (br, 1H), 10.10 (br, 1H), 9.02 (s, 1H), 8.85 (s, 2H), 8.26 (s, 1H), 7.79 (s, 1H), 7.70 (dd, J=7.6, 1.2 Hz, 1H), 7.61-7.50 (m, 3H), 7.04 (d, J=8.8 Hz, 2H), 4.73-4.69 (m, 2H), 4.08-4.04 (m, 2H), 3.32-3.32 (m, 4H), 3.26-3.25 (m, 4H). LCMS (M+H⁺) m/z: 458.0.

### Example 88: Preparation of 6-(2-chlorophenyl)-N-(2-methoxy-4-(piperazin-1-yl)phenyl)-8,9-dihydroimidazo[1′,2′:1,6]pyrido[2,3-d]pyrimidin-2-amine (Compound 88)

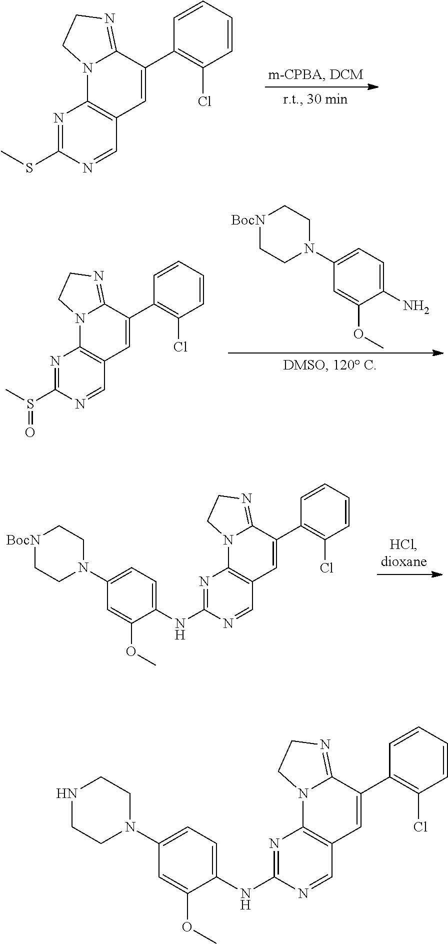 [SMILES: CSc1ncc2c(n1)N1CCN=C1C(c1ccccc1Cl)=C2 ; CS(=O)c1ncc2c(n1)N1CCN=C1C(c1ccccc1Cl)=C2 ; COc1cc(N2CCN(C(=O)OC(C)(C)C)CC2)ccc1N ; COc1cc(N2CCN(C(=O)OC(C)(C)C)CC2)ccc1Nc1ncc2c(n1)N1CCN=C1C(c1ccccc1Cl)=C2 ; COc1cc(N2CCNCC2)ccc1Nc1ncc2c(n1)N1CCN=C1C(c1ccccc1Cl)=C2]

### Step 1: Synthesis of 6-(2-chlorophenyl)-2-(methylsulfinyl)-8,9-dihydroimidazo[1′,2′:1,6]pyrido[2,3-d]pyrimidine

To a solution of 6-(2-chlorophenyl)-2-(methylthio)-8,9-dihydroimidazo[1′,2′:1,6]pyrido[2,3-d]pyrimidine (400 mg, 1.2 mmol) in DCM (8 mL) was added m-CPBA (416 mg, 2.4 mmol), the reaction mixture was stirred at 0° C. for 15 min. LCMS showed the reaction completed. The reaction mixture was concentrated to obtain 6-(2-chlorophenyl)-2-(methylsulfinyl)-8,9-dihydroimidazo[1′,2′:1,6]pyrido[2,3-d]pyrimidine as yellow solid (400 mg, crude), which was used for next step without further purification. LCMS (M+H⁺) m/z: 344.9.

### Step 2: Synthesis of tert-butyl 4-(4-((6-(2-chlorophenyl)-8,9-dihydroimidazo[1′,2′:1,6]pyrido[2,3-d]pyrimidin-2-yl)amino)-3-methoxyphenyl)piperazine-1-carboxylate

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added tert-butyl 4-(4-amino-3-methoxyphenyl)piperazine-1-carboxylate (460 mg, 1.5 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by chromatography column (DCM:MeOH=20:1) to afford tert-butyl 4-(4-((6-(2-chlorophenyl)-8,9-dihydroimidazo[1′,2′:1,6]pyrido[2,3-d]pyrimidin-2-yl)amino)-3-methoxyphenyl)piperazine-1-carboxylate (45 mg) as yellow solid. LCMS (M+H⁺) m/z: 588.0.

### Step 3: Synthesis of 6-(2-chlorophenyl)-N-(2-methoxy-4-(piperazin-1-yl)phenyl)-8,9-dihydroimidazo[1′,2′:1,6]pyrido[2,3-d]pyrimidin-2-amine

To a solution of tert-butyl 4-(4-((6-(2-chlorophenyl)-8,9-dihydroimidazo[1′,2′:1,6]pyrido[2,3-d]pyrimidin-2-yl)amino)-3-methoxyphenyl)piperazine-1-carboxylate (45 mg, 0.08 mmol) in dioxane (1 mL) was added HCl (4M in dioxane) (1 mL). The mixture was stirred at 25° C. for 3 h. LCMS showed the reaction completed. The mixture was concentrated to afford a crude solid, which was purified by prep-HPLC to afford 6-(2-chlorophenyl)-N-(2-methoxy-4-(piperazin-1-yl)phenyl)-8,9-dihydroimidazo[1′,2′:1,6]pyrido[2,3-d]pyrimidin-2-amine (25.1 mg) as red solid. ¹H NMR (400 MHz, DMSO-d₆): δ 8.28 (s, 1H), 8.23 (s, 3H), 7.84 (br, 1H), 7.54-7.49 (m, 1H), 7.44-7.36 (m, 3H), 7.25 (s, 1H), 6.67 (d, J=2.4 Hz, 1H), 6.53 (d, J=8.8, 2.4 Hz, 1H), 4.03 (d, J=9.2 Hz, 2H), 3.92 (d, J=9.2 Hz, 2H), 3.83 (s, 3H), 3.22 (s, 4H), 3.07 (s, 4H). LCMS (M+H⁺) m/z: 488.0.

### Example 89: Preparation of 6-(2-chlorophenyl)-N-(4-morpholinophenyl)-8,9-dihydroimidazo[1′,2′:1,6]pyrido[2,3-d]pyrimidin-2-amine (Compound 89)

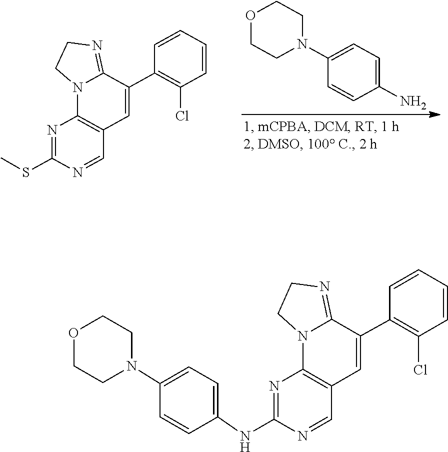 [SMILES: CSc1ncc2c(n1)N1CCN=C1C(c1ccccc1Cl)=C2 ; Nc1ccc(N2CCOCC2)cc1 ; Clc1ccccc1C1=Cc2cnc(Nc3ccc(N4CCOCC4)cc3)nc2N2CCN=C12]

### Step 1: Synthesis of 6-(2-chlorophenyl)-N-(4-morpholinophenyl)-8,9-dihydroimidazo[1′,2′:1,6]pyrido[2,3-d]pyrimidin-2-amine

To a mixture of 6-(2-chlorophenyl)-2-(methylthio)-8,9-dihydroimidazo[1′,2′:1,6]pyrido[2,3-d]pyrimidine (100 mg, 0.30 mmol) in DCM (2 mL) was added m-CPBA (155 mg, 0.90 mmol) at room temperature and the reaction mixture was stirred for 20 min. The mixture was concentrated and 4-morpholinoaniline (267 mg, 1.5 mmol) in DMSO (0.2 mL) was added. The reaction mixture was stirred at 100° C. for 2 h. The reaction mixture was purified by prep-HPLC to give 6-(2-chlorophenyl)-N-(4-morpholinophenyl)-8,9-dihydroimidazo[1′,2′:1,6]pyrido[2,3-d]pyrimidin-2-amine (86.5 mg, 62.8% yield) as a red solid. ¹H NMR (400 MHz, DMSO-d₆): δ 9.67 (s, 1H), 8.33 (s, 1H), 8.17 (s, 1H), 7.67 (d, J=8.8 Hz, 2H), 7.54-7.51 (m, 1H), 7.45-7.37 (m, 3H), 7.26 (s, 1H), 6.91 (d, J=9.2 Hz, 2H), 4.08 (t, J=9.6 Hz, 2H), 3.93 (t, J=9.6 Hz, 2H), 3.74 (t, J=4.8 Hz, 4H), 3.04 (t, J=4.8 Hz, 4H). LCMS (M+H⁺) m/z: 458.9.

### Example 90: Preparation of 6-(2-chlorophenyl)-N-(4-(4-methylpiperazin-1-yl)phenyl)-8,9-dihydroimidazo[1′,2′:1,6]pyrido[2,3-d]pyrimidin-2-amine (Compound 90)

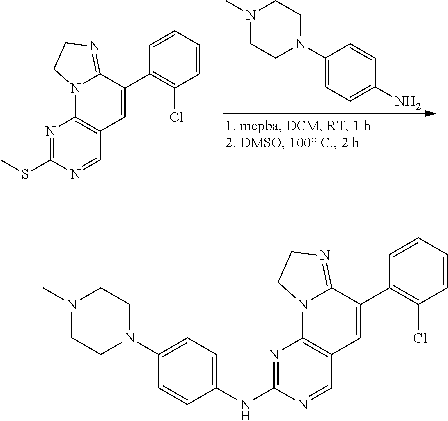 [SMILES: CSc1ncc2c(n1)N1CCN=C1C(c1ccccc1Cl)=C2 ; CN1CCN(c2ccc(N)cc2)CC1 ; CN1CCN(c2ccc(Nc3ncc4c(n3)N3CCN=C3C(c3ccccc3Cl)=C4)cc2)CC1]

### Step 1: Synthesis of 6-(2-chlorophenyl)-N-(4-(4-methylpiperazin-1-yl)phenyl)-8,9-dihydroimidazo[1′,2′:1,6]pyrido[2,3-d]pyrimidin-2-amine

The mixture of 6-(2-chlorophenyl)-2-(methylthio)-8,9-dihydroimidazo[1′,2′:1,6]pyrido[2,3-d]pyrimidine (100 mg, 0.3 mmol) and m-CPBA (130 mg, 0.75 mmol) in DCM (3 mL) was stirred for 0.5 h at 25° C. LCMS showed the reaction was completed. The reaction was concentrated and a solution of 4-(4-methylpiperazin-1-yl)aniline (286 mg, 1.5 mmol) in DMSO (0.5 mL) was added to reaction mixture. The reaction was stirred at 100° C. for 2 h. Purification by HPLC (0.5% FA) gave 6-(2-chlorophenyl)-N-(4-(4-methylpiperazin-1-yl)phenyl)-8,9-dihydroimidazo[1′,2′:1,6]pyrido[2,3-d]pyrimidin-2-amine (54.2 mg) as yellow solid. ¹H NMR (400 MHz, DMSO-d₆): δ 9.65 (s, 1H), 8.32 (s, 1H), 7.65 (d, J=8.4 Hz, 2H), 7.53-7.51 (m, 1H), 7.44-7.37 (m, 3H), 7.25 (s, 1H), 6.89 (d, J=9.2 Hz, 2H), 4.11-4.07 (m, 2H), 3.95-3.90 (m, 2H), 3.08-3.06 (m, 4H), 2.49-2.45 (m, 4H), 2.22 (s, 3H). LCMS (M+H⁺) m/z: 472.0.

### Example 91: Preparation of 6-(2-chlorophenyl)-N-(2-methoxy-4-(4-methylpiperazin-1-yl)phenyl)-8,9-dihydroimidazo[1′,2′:1,6]pyrido[2,3-d]pyrimidin-2-amine (Compound 91)

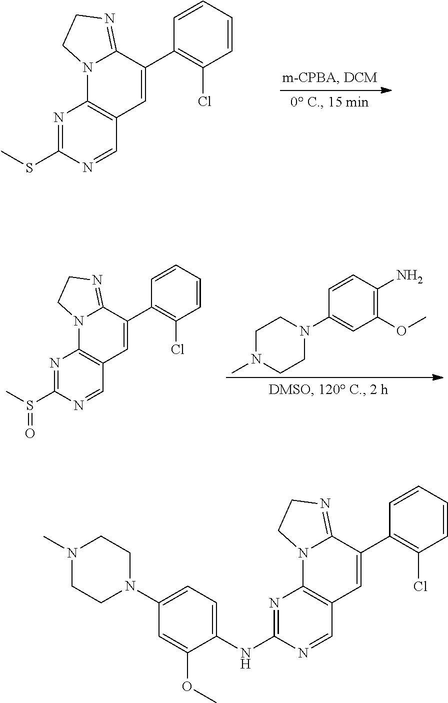

### Step 1: Synthesis of 6-(2-chlorophenyl)-2-(methylsulfinyl)-8,9-dihydroimidazo[1′,2′:1,6]pyrido[2,3-d]pyrimidine

To a solution of 6-(2-chlorophenyl)-2-(methylthio)-8,9-dihydroimidazo[1′,2′:1,6]pyrido[2,3-d]pyrimidine (400 mg, 1.2 mmol) in DCM (8 mL) was added m-CPBA (416 mg, 2.4 mmol), the reaction mixture was stirred at 0° C. for 15 min. LCMS showed the reaction completed. The reaction mixture was concentrated to obtain a yellow solid (400 mg, crude), which was used for next step without further purification. LCMS (M+H⁺) m/z: 362.0.

### Step 2: Synthesis of 6-(2-chlorophenyl)-N-(2-methoxy-4-(4-methylpiperazin-1-yl)phenyl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 2-methoxy-4-(4-methylpiperazin-1-yl)aniline (330 mg, 1.5 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford 6-(2-chlorophenyl)-N-(2-methoxy-4-(4-methylpiperazin-1-yl)phenyl)-8,9-dihydroimidazo[1′,2′:1,6]pyrido[2,3-d]pyrimidin-2-amine (36.5 mg) as yellow solid. ¹H NMR (400 MHz, DMSO-d₆): δ 8.31 (s, 1H), 8.27 (s, 1H), 8.20 (s, 2H), 7.77 (br, 1H), 7.54-7.52 (m, 1H), 7.44-7.37 (m, 3H), 7.29 (s, 1H), 6.64 (d, J=2.0 Hz, 1H), 6.50 (dd, J=8.8, 2.4 Hz, 1H), 4.07 (t, J=9.6 Hz, 2H), 3.91 (t, J=9.6 Hz, 2H), 3.83 (d, J=9.6 Hz, 3H), 3.21-3.08 (m, 4H), 2.54-2.52 (m, 4H), 2.27 (s, 3H). LCMS (M+H⁺) m/z: 502.1.

### Example 92: Preparation of 6-(2-chlorophenyl)-N-(6-(4-methylpiperazin-1-yl)pyridin-3-yl)-8,9-dihydroimidazo[1′,2′:1,6]pyrido[2,3-d]pyrimidin-2-amine (Compound 92)

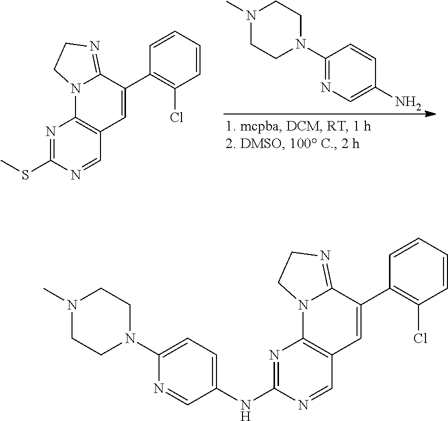 [SMILES: CSc1ncc2c(n1)N1CCN=C1C(c1ccccc1Cl)=C2 ; CN1CCN(c2ccc(N)cn2)CC1 ; CN1CCN(c2ccc(Nc3ncc4c(n3)N3CCN=C3C(c3ccccc3Cl)=C4)cn2)CC1]

### Step 1: Synthesis of 6-(2-chlorophenyl)-N-(6-(4-methylpiperazin-1-yl)pyridin-3-yl)-8,9-dihydroimidazo[1′,2′:1,6]pyrido[2,3-d]pyrimidin-2-amine

To a mixture of 6-(2-chlorophenyl)-2-(methylthio)-8,9-dihydroimidazo[1′,2′:1,6]pyrido[2,3-d]pyrimidine (60 mg, 0.18 mmol) in DCM (2 mL) was added m-CPBA (93 mg, 0.54 mmol) at room temperature and stirred for 20 min. The mixture was concentrated and 6-(4-methylpiperazin-1-yl)pyridin-3-amine (173 mg, 0.9 mmol) in DMSO (0.2 mL). was added. The reaction mixture was stirred at 100° C. for 2 h. Purification by prep-HPLC gave 6-(2-chlorophenyl)-N-(6-(4-methylpiperazin-1-yl)pyridin-3-yl)-8,9-dihydroimidazo[1′,2′:1,6]pyrido[2,3-d]pyrimidin-2-amine (58.3 mg, 68.5% yield) as brown solid. ¹H NMR (400 MHz, DMSO-d₆): δ 9.66 (s, 1H), 8.52 (s, 1H), 8.33 (s, 1H), 7.93 (d, J=7.2 Hz, 1H), 7.53-7.51 (m, 1H), 7.44-7.37 (m, 3H), 7.27 (s, 1H), 6.83 (d, J=9.2 Hz, 1H), 4.09 (t, J=9.6 Hz, 2H), 3.93 (t, J=8.8 Hz, 2H), 3.42 (t, J=4.8 Hz, 4H), 2.43 (t, J=5.2 Hz, 4H), 2.23 (s, 3H). LCMS (M+H⁺) m/z: 472.7.

### Example 93: Preparation of 6-(2-chlorophenyl)-N-(3-(piperazin-1-yl)phenyl)-8,9-dihydroimidazo[1′,2′:1,6]pyrido[2,3-d]pyrimidin-2-amine (Compound 93)

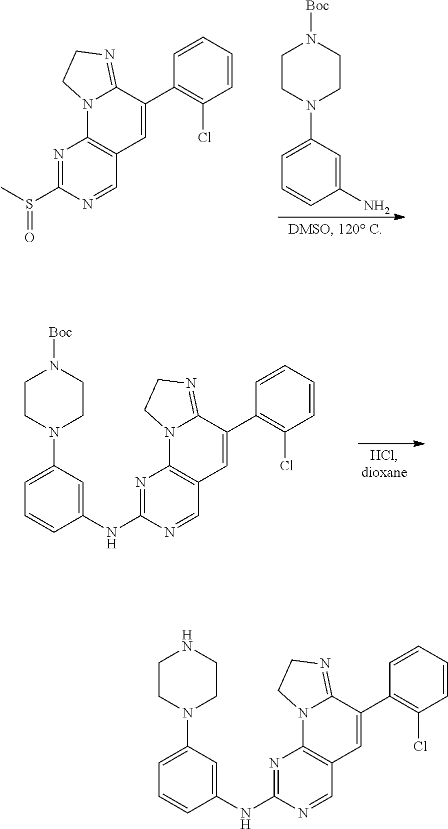 [SMILES: CS(=O)c1ncc2c(n1)N1CCN=C1C(c1ccccc1Cl)=C2 ; CC(C)(C)OC(=O)N1CCN(c2cccc(N)c2)CC1 ; CC(C)(C)OC(=O)N1CCN(c2cccc(Nc3ncc4c(n3)N3CCN=C3C(c3ccccc3Cl)=C4)c2)CC1 ; Clc1ccccc1C1=Cc2cnc(Nc3cccc(N4CCNCC4)c3)nc2N2CCN=C12]

### Step 1: Synthesis of tert-butyl 4-(3-((6-(2-chlorophenyl)-8,9-dihydroimidazo[1′,2′:1,6]pyrido[2,3-d]pyrimidin-2-yl)amino)phenyl)piperazine-1-carboxylate

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added tert-butyl 4-(3-aminophenyl)piperazine-1-carboxylate (554 mg, 2 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford tert-butyl 4-(3-((6-(2-chlorophenyl)-8,9-dihydroimidazo[1′,2′:1,6]pyrido[2,3-d]pyrimidin-2-yl)amino)phenyl)piperazine-1-carboxylate (52 mg, 28% yield) as yellow solid. LCMS (M+H⁺) m/z: 558.0.

### Step 2: Synthesis of 6-(2-chlorophenyl)-N-(3-(piperazin-1-yl)phenyl)-8,9-dihydroimidazo[1′,2′:1,6]pyrido[2,3-d]pyrimidin-2-amine

To a solution of tert-butyl 4-(3-((6-(2-chlorophenyl)-8,9-dihydroimidazo[1′,2′:1,6]pyrido[2,3-d]pyrimidin-2-yl)amino)phenyl)piperazine-1-carboxylate (40 mg, 0.07 mmol) in dioxane (2 mL) was added HCl (4M in dioxane) (1 mL, 4 mmol). The mixture was stirred at 25° C. for 2 h. LCMS showed the reaction completed. The mixture was concentrated to afford a crude solid, which was purified by prep-HPLC to afford 6-(2-chlorophenyl)-N-(3-(piperazin-1-yl)phenyl)-8,9-dihydroimidazo[1′,2′:1,6]pyrido[2,3-d]pyrimidin-2-amine (11.8 mg) as yellow solid. ¹H NMR (400 MHz, DMSO-d₆): δ 9.70 (s, 1H), 8.36 (s, 1H), 7.59 (br, 1H), 7.55-7.52 (m, 1H), 7.45-7.42 (m, 1H), 7.41-7.36 (m, 1H), 7.26 (s, 1H), 7.21 (d, J=8.0 Hz, 1H), 7.12 (t, J=8.0 Hz, 1H), 6.59-6.56 (m, 1H), 6.10-6.08 (m, 2H), 4.12 (t, J=9.6 Hz, 2H), 3.95 (t, J=9.6 Hz, 2H), 3.72-3.66 (m, 2H), 3.59-3.57 (m, 2H), 3.08-3.06 (m, 4H). LCMS (M+H⁺) m/z: 458.0.

### Example 94: Preparation of 6-(2-chlorophenyl)-N-(3-morpholinophenyl)-8,9-dihydroimidazo[1′,2′:1,6]pyrido[2,3-d]pyrimidin-2-amine (Compound 94)

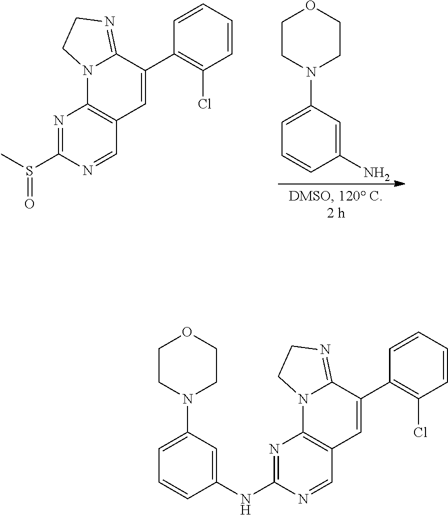 [SMILES: CS(=O)c1ncc2c(n1)N1CCN=C1C(c1ccccc1Cl)=C2 ; Nc1cccc(N2CCOCC2)c1 ; Clc1ccccc1C1=Cc2cnc(Nc3cccc(N4CCOCC4)c3)nc2N2CCN=C12]

### Step 1: Synthesis of 6-(2-chlorophenyl)-N-(3-morpholinophenyl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 3-morpholinoaniline (356 mg, 2 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford 6-(2-chlorophenyl)-N-(3-morpholinophenyl)-8,9-dihydroimidazo[1′,2′:1,6]pyrido[2,3-d]pyrimidin-2-amine (47.1 mg) as yellow solid. ¹H NMR (400 MHz, DMSO-d₆): δ 9.75 (s, 1H), 8.38 (s, 1H), 8.16 (s, 1H), 7.59 (s, 1H), 7.55-7.52 (m, 1H), 7.46-7.37 (m, 3H), 7.29 (s, 1H), 7.23 (d, J=8.0 Hz, 1H), 7.14 (t, J=8.0 Hz, 1H), 4.13 (t, J=9.6 Hz, 2H), 3.95 (t, J=9.6 Hz, 2H), 3.77-3.73 (m, 4H), 3.13-3.07 (m, 4H). LCMS (M+H⁺) m/z: 459.0.

### Example 95: Preparation of 6-(2-chlorophenyl)-N-(2-methoxy-5-(4-methylpiperazin-1-yl)phenyl)-8,9-dihydroimidazo[1′,2′:1,6]pyrido[2,3-d]pyrimidin-2-amine (Compound 95)

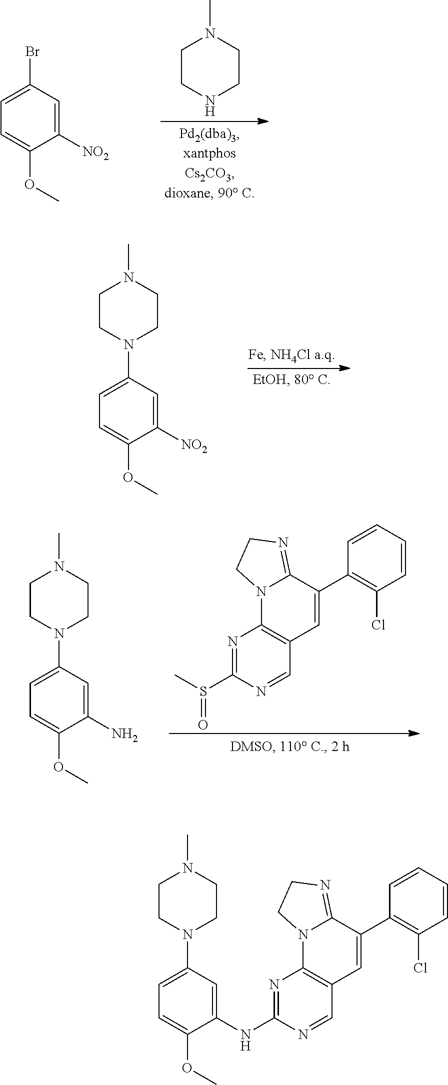

### Step 1: Synthesis of 1-(4-methoxy-3-nitrophenyl)-4-methylpiperazine

To a solution of 4-bromo-1-methoxy-2-nitrobenzene (1.3 g, 5.65 mmol) in dioxane (15 mL) was added 1-methylpiperazine (565 mg, 5.65 mmol), Pd₂ (dba) 3 (510 mg, 0.56 mmol), Cs₂CO₃ (3.67 g, 11.3 mmol) and xant-phos (323 mg, 0.56 mmol), the reaction mixture was stirred at 90° C. for 3 h. LCMS showed the reaction completed. The mixture was diluted to water (20 mL), extracted by EA (20 mL*3). The combined organic phase was washed by brine (50 mL), dried over anhydrous Na₂SO₄, filtered and concentrated to afford a crude solid, which was purified by chromatography column (DCM:MeOH=20:1) to afford 1-(4-methoxy-3-nitrophenyl)-4-methylpiperazine (1 g, 71% yield) as white solid. LCMS (M+H⁺) m/z: 251.9.

### Step 2: Synthesis of 2-methoxy-5-(4-methylpiperazin-1-yl)aniline

To a solution of 1-(4-methoxy-3-nitrophenyl)-4-methylpiperazine (1 g, 4 mmol) in EtOH (15 mL) was added Fe powder (448 mg, 8 mmol) and saturated NH₄Cl a.q. (3 mL), the reaction mixture was stirred at 80° C. for 2 h. LCMS showed the reaction completed. The reaction mixture was filtered by celite, concentrated to obtain a yellow solid, which was purified by prep-HPLC to afford 2-methoxy-5-(4-methylpiperazin-1-yl)aniline (440 mg, 50% yield) as a gray solid. LCMS (M+H⁺) m/z: 222.1.

### Step 3: Synthesis of 6-(2-chlorophenyl)-N-(2-methoxy-5-(4-methylpiperazin-1-yl)phenyl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 2-methoxy-5-(4-methylpiperazin-1-yl)aniline (331 mg, 1.5 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford 6-(2-chlorophenyl)-N-(2-methoxy-5-(4-methylpiperazin-1-yl)phenyl)-8,9-dihydroimidazo[1′,2′:1,6]pyrido[2,3-d]pyrimidin-2-amine (49 mg) as brown solid. ¹H NMR (400 MHz, DMSO-d₆): δ 10.16 (s, 1H), 9.59 (s, 1H), 9.01 (s, 1H), 8.28 (s, 1H), 7.70 (dd, J=8.0, 1.2 Hz, 1H), 7.60-7.49 (m, 3H), 7.05 (d, J=9.2 Hz, 1H), 6.88 (dd, J=8.8, 2.8 Hz, 1H), 4.64-4.60 (m, 2H), 4.04-4.00 (m, 2H), 3.79 (s, 3H), 3.72-3.69 (m, 2H), 3.54 (d, J=11.2 Hz, 2H), 3.20-3.16 (m, 2H), 2.97-2.91 (m, 2H), 2.87 (s, 3H). LCMS (M+H⁺) m/z: 502.0.

### Example 96: Preparation of 6-(2-chlorophenyl)-N-(3-(4-methylpiperazin-1-yl)phenyl)-8,9-dihydroimidazo[1′,2′:1,6]pyrido[2,3-d]pyrimidin-2-amine (Compound 96)

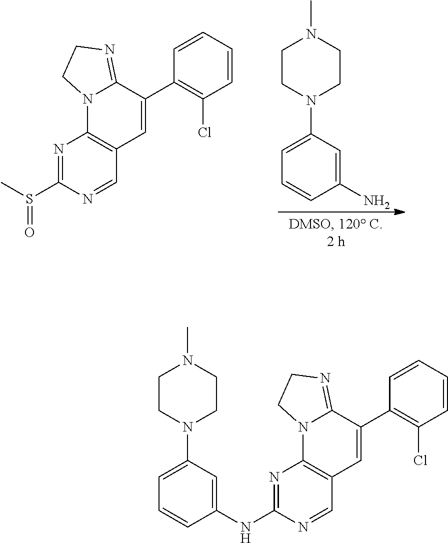 [SMILES: CS(=O)c1ncc2c(n1)N1CCN=C1C(c1ccccc1Cl)=C2 ; CN1CCN(c2cccc(N)c2)CC1 ; CN1CCN(c2cccc(Nc3ncc4c(n3)N3CCN=C3C(c3ccccc3Cl)=C4)c2)CC1]

### Step 1: Synthesis of 6-(2-chlorophenyl)-N-(3-(4-methylpiperazin-1-yl)phenyl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 3-(4-methylpiperazin-1-yl)aniline (382 mg, 2 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford 6-(2-chlorophenyl)-N-(3-(4-methylpiperazin-1-yl)phenyl)-8,9-dihydroimidazo[1′,2′:1,6]pyrido[2,3-d]pyrimidin-2-amine (38.2 mg) as yellow solid. ¹H NMR (400 MHz, DMSO-d₆): δ 9.70 (s, 1H), 8.37 (s, 1H), 8.20 (s, 1H), 7.61 (s, 1H), 7.57-7.49 (m, 1H), 7.47-7.35 (m, 3H), 7.27 (s, 1H), 7.19 (d, J=8.8 Hz, 1H), 7.11 (t, J=8.0 Hz, 1H), 6.59 (d, J=8.0 Hz, 1H), 4.12 (t, J=9.6 Hz, 2H), 3.95 (t, J=9.6 Hz, 2H), 3.19-3.07 (m, 4H), 2.49-2.44 (m, 4H), 2.23 (s, 3H). LCMS (M+H⁺) m/z: 471.9.

### Example 97: Preparation of 1-(3-((6-(2-chlorophenyl)-8,9-dihydroimidazo[1′,2′:1,6]pyrido[2,3-d]pyrimidin-2-yl)amino)phenyl)ethan-1-ol (Compound 97)

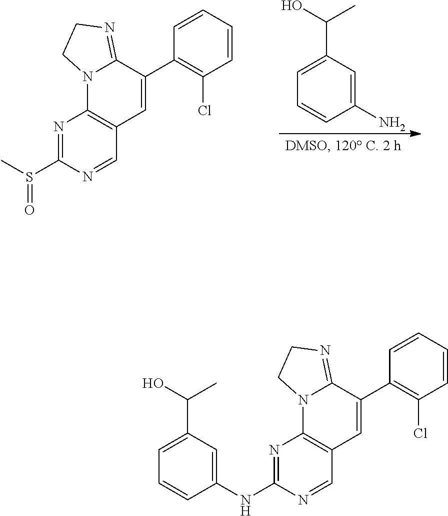

### Step 1: Synthesis of 1-(3-((6-(2-chlorophenyl)-8,9-dihydroimidazo[1′,2′:1,6]pyrido[2,3-d]pyrimidin-2-yl)amino)phenyl)ethan-1-ol

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 1-(3-aminophenyl)ethan-1-ol (274 mg, 2 mmol). The mixture was stirred at 120° C. for 2 h. LCMS showed the reaction completed. The mixture was diluted to water (10 mL), extracted by EA (10 mL*3), combined organic phase was washed by brine (20 mL), dried over anhydrous Na₂SO₄, filtered and concentrated to afford a crude solid, which was purified by prep-HPLC to afford 1-(3-((6-(2-chlorophenyl)-8,9-dihydroimidazo[1′,2′:1,6]pyrido[2,3-d]pyrimidin-2-yl)amino)phenyl)ethan-1-ol (24.4 mg, 20% yield) as yellow solid. ¹H NMR (400 MHz, DMSO-d₆): δ 9.85 (s, 1H), 8.39 (s, 1H), 8.16 (s, 1H), 7.92 (s, 1H), 7.62 (d, J=8.0 Hz, 1H), 7.56-7.50 (m, 1H), 7.48-7.36 (m, 3H), 7.30 (s, 1H), 7.23 (t, J=7.6 Hz, 1H), 6.97 (d, J=7.6 Hz, 1H), 5.14 (br, 1H), 4.69 (q, J=6.4 Hz, 1H), 4.14 (t, J=9.6 Hz, 2H), 3.95 (t, J=9.6 Hz, 2H), 1.34 (d, J=6.4 Hz, 3H). LCMS (M+H⁺) m/z: 418.0.

### Example 98: Preparation of 2-(3-((6-(2-chlorophenyl)-8,9-dihydroimidazo[1′,2′:1,6]pyrido[2,3-d]pyrimidin-2-yl)amino)phenyl)propan-2-ol (Compound 98)

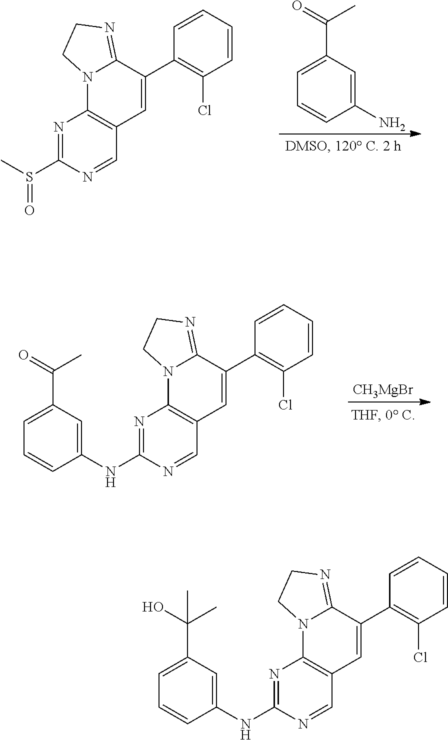

### Step 1: Synthesis of 1-(3-((6-(2-chlorophenyl)-8,9-dihydroimidazo[1′,2′:1,6]pyrido[2,3-d]pyrimidin-2-yl)amino)phenyl)ethan-1-one

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 1-(3-aminophenyl)ethan-1-one (270 mg, 2 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by Prep-HPLC to afford 1-(3-((6-(2-chlorophenyl)-8,9-dihydroimidazo[1′,2′:1,6]pyrido[2,3-d]pyrimidin-2-yl)amino)phenyl)ethan-1-one (80 mg, 64% yield) as yellow solid. LCMS (M+H⁺) m/z: 416.0.

### Step 2: Synthesis of 2-(3-((6-(2-chlorophenyl)-8,9-dihydroimidazo[1′,2′:1,6]pyrido[2,3-d]pyrimidin-2-yl)amino)phenyl)propan-2-ol

To a solution of 1-(3-((6-(2-chlorophenyl)-8,9-dihydroimidazo[1′,2′:1,6]pyrido[2,3-d]pyrimidin-2-yl)amino)phenyl)ethan-1-one (80 mg, 0.19 mmol) in THF (3 mL) was added CH3MgBr (1 mL, 1 mmol). The mixture was stirred at 0° C. for 2 h. LCMS showed the reaction completed. The mixture was diluted to water (10 mL), extracted by EA (10 mL*3). The combined organic phase was washed by brine (20 mL), dried over anhydrous Na2SO4, filtered and concentrated to afford a crude solid, which was purified by prep-HPLC to afford 2-(3-((6-(2-chlorophenyl)-8,9-dihydroimidazo[1′,2′:1,6]pyrido[2,3-d]pyrimidin-2-yl)amino)phenyl)propan-2-ol (14.4 mg) as yellow solid. ¹H NMR (400 MHz, DMSO-d6): δ 9.82 (s, 1H), 8.38 (s, 1H), 8.15 (s, 1H), 8.05 (s, 1H), 7.58 (d, J=8.0 Hz, 1H), 7.55-7.50 (m, 1H), 7.44 (dt, J=4.0, 3.6 Hz, 1H), 7.41-7.36 (m, 2H), 7.30 (s, 1H), 7.22 (t, J=8.0 Hz, 1H), 7.10 (d, J=8.0 Hz, 1H), 4.96 (s, 1H), 4.14 (t, J=9.6 Hz, 2H), 3.95 (t, J=9.6 Hz, 2H), 1.44 (s, 6H). LCMS (M+H⁺) m/z: 432.0.

### Example 99: Preparation of ethyl 3-((6-(2-chlorophenyl)-8,9-dihydroimidazo[1′,2′:1,6]pyrido[2,3-d]pyrimidin-2-yl)amino)benzoate (Compound 99)

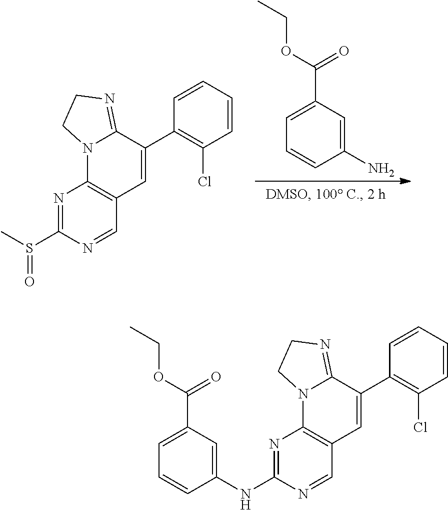 [SMILES: CS(=O)c1ncc2c(n1)N1CCN=C1C(c1ccccc1Cl)=C2 ; CCOC(=O)c1cccc(N)c1 ; CCOC(=O)c1cccc(Nc2ncc3c(n2)N2CCN=C2C(c2ccccc2Cl)=C3)c1]

### Step 1: Synthesis of 3-((6-(2-chlorophenyl)-8,9-dihydroimidazo[1′,2′:1,6]pyrido[2,3-d]pyrimidin-2-yl)amino)benzoate

To a solution of 6-(2-chlorophenyl)-2-(methylsulfinyl)-8,9-dihydroimidazo[1′,2′:1,6]pyrido[2,3-d]pyrimidine (50 mg, 0.145 mmol) and ethyl 3-aminobenzoate (479 mg, 2.9 mmol) in DMSO (10 drops) was added TEA (29 mg, 0.29 mmol). The mixture was stirred at 100° C. for 2 h. The mixture was purified by prep-HPLC to give 3-((6-(2-chlorophenyl)-8,9-dihydroimidazo[1′,2′:1,6]pyrido[2,3-d]pyrimidin-2-yl)amino)benzoate (17.5 mg, 27% yield) as white solid. ¹H NMR (400 MHz, DMSO-d₆): δ 10.14 (s, 1H), 8.77 (s, 1H), 8.43 (s, 1H), 7.94 (d, J=8.0 Hz, 1H), 7.60-7.52 (m, 2H), 7.47-7.37 (m, 4H), 7.33 (s, 1H), 4.32 (q, J=7.2 Hz, 2H), 4.19 (t, J=9.6 Hz, 2H), 3.98 (t, J=9.6 Hz, 2H), 1.33 (t, J=7.2 Hz, 3H). LCMS (M+H⁺) m/z: 446.0.

### Example 100: Preparation of 6-(2-chlorophenyl)-N-(6-morpholinopyridin-3-yl)-8,9-dihydroimidazo[1′,2′:1,6]pyrido[2,3-d]pyrimidin-2-amine (Compound 100)

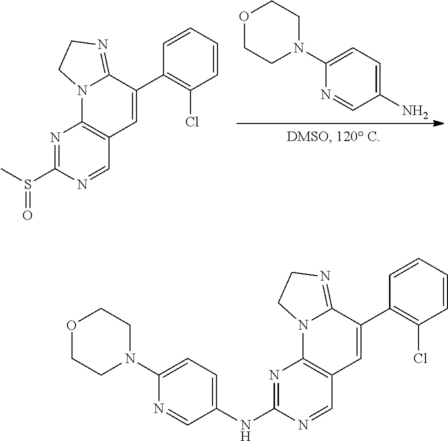

### Step 1: Synthesis of 6-(2-chlorophenyl)-N-(6-morpholinopyridin-3-yl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 6-morpholinopyridin-3-amine (358 mg, 2 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na2SO4, filtered and concentrated to afford a crude solid, which was purified by prep-HPLC to afford 6-(2-chlorophenyl)-N-(6-morpholinopyridin-3-yl)-8,9-dihydroimidazo[1′,2′:1,6]pyrido[2,3-d]pyrimidin-2-amine (38.3 mg, 29% yield) as yellow solid. ¹H NMR (400 MHz, DMSO-d6): δ 9.69 (s, 1H), 8.54 (s, 1H), 8.34 (s, 1H), 8.17 (s, 1H), 7.97 (d, J=7.6 Hz, 1H), 7.54-7.51 (m, 1H), 7.44-7.36 (m, 3H), 7.27 (s, 1H), 6.84 (d, J=9.2 Hz, 1H), 4.09 (t, J=9.6 Hz, 2H), 3.93 (t, J=9.6 Hz, 2H), 3.71 (t, J=4.8 Hz, 4H), 3.37 (t, J=4.8 Hz, 4H). LCMS (M+H⁺) m/z: 460.0.

### Example 101: Preparation of 6-(2-chlorophenyl)-N-(6-methylpyridin-3-yl)-8,9-dihydroimidazo[1′,2′:1,6]pyrido[2,3-d]pyrimidin-2-amine (Compound 101)

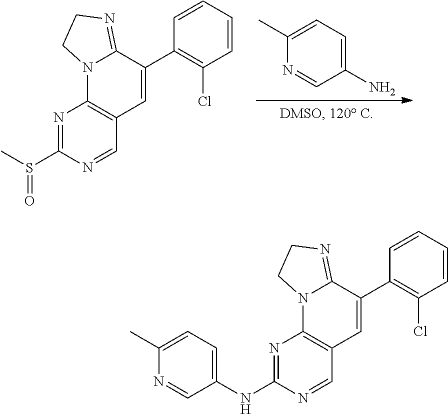

### Step 1: Synthesis of 6-(2-chlorophenyl)-N-(6-methylpyridin-3-yl)-8,9-dihydroimidazo[1′,2′:1,6]pyrido[2,3-d]pyrimidin-2-amine

To a solution of 6-(2-chlorophenyl)-2-(methylsulfinyl)-8,9-dihydroimidazo[1′,2′:1,6]pyrido[2,3-d]pyrimidine (100 mg, crude) in DMSO (8 drops) was added 6-methylpyridin-3-amine (162 mg, 1.5 mmol). The mixture was stirred at 120° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford 6-(2-chlorophenyl)-N-(6-methylpyridin-3-yl)-8,9-dihydroimidazo[1′,2′:1,6]pyrido[2,3-d]pyrimidin-2-amine (29.9 mg) as yellow solid. ¹H NMR (400 MHz, DMSO-d₆): δ 9.93 (s, 1H), 8.83 (d, J=2.4 Hz, 1H), 8.39 (s, 1H), 8.17 (s, 1H), 8.11 (dd, J=8.4, 2.4 Hz, 1H), 7.54-7.52 (m, 1H), 7.45-7.38 (m, 3H), 7.30 (s, 1H), 7.19 (d, J=8.8 Hz, 1H), 4.12 (t, J=9.6 Hz, 2H), 3.94 (t, J=9.6 Hz, 2H), 2.41 (s, 3H). LCMS (M+H⁺) m/z: 389.0.

### Example 102: Preparation of 6-(2,4-dichlorophenyl)-N-(1-methyl-1H-pyrazol-4-yl)-8,9-dihydroimidazo[1′,2′:1,6]pyrido[2,3-d]pyrimidin-2-amine (Compound 102)

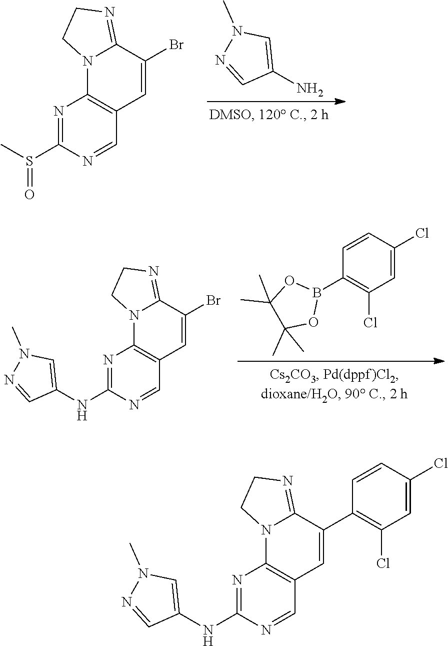

### Step 1: Synthesis of 6-bromo-N-(6-methylpyridin-3-yl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (6.0 g, 19.17 mmol) in DMSO (30 mL) was added 1-methyl-1H-pyrazol-4-amine (2.8 g, 28.75 mmol), the mixture was stirred for 2 h at 120° C. The reaction mixture was removed in vacuum. The residue was purified by column chromatography (DCM:MeOH=20:1) to afford 6-bromo-N-(6-methylpyridin-3-yl)-8,9-dihydroimidazo[1′,2′:1,6]pyrido[2,3-d]pyrimidin-2-amine (6.0 g, 85.7% yield) as a brown solid. LCMS (M+H⁺) m/z: 346.0 and 348.0.

### Step 2: Synthesis of 6-(2,4-dichlorophenyl)-N-(1-methyl-1H-pyrazol-4-yl)-8,9-dihydroimidazo[1′,2′:1,6]pyrido[2,3-d]pyrimidin-2-amine

To a solution of 6-bromo-N-(1-methyl-1H-pyrazol-4-yl)-8,9-dihydroimidazo[1′,2′:1,6]pyrido[2,3-d]pyrimidin-2-amine (100 mg, 0.29 mmol) in dioxane/H₂O (6 mL/2 mL) was added 2-(2,4-dichlorophenyl)-4,4,5,5-tetramethyl-1,3,2-dioxaborolane (55 mg, 0.29 mmol), Pd(dppf)Cl₂ (21 mg, 0.029 mmol), Cs₂CO₃ (283 mg, 0.84 mmol). The mixture was stirred for 2 h at 90° C. under N2. The reaction mixture was concentrated and the residue purified by Prep-HPLC (0.1% FA) to afford 6-(2,4-dichlorophenyl)-N-(1-methyl-1H-pyrazol-4-yl)-8,9-dihydroimidazo[1′,2′:1,6]pyrido[2,3-d]pyrimidin-2-amine (11.2 mg, 9.4% yield) as a yellow solid. ¹H NMR (400 MHz, DMSO-d₆): δ 9.87 (s, 0.7H), 9.73 (s, 0.3H), 8.33 (s, 1H), 8.19 (s, 1H), 7.92 (s, 1H), 7.69 (s, 1H), 7.56 (s, 1H), 7.47 (s, 2H), 7.30 (s, 1H), 4.22-4.18 (m, 2H), 3.95 (t, J=8.4 Hz, 2H), 3.82 (s, 3H). LCMS (M+H⁺) m/z: 412.1.

### Example 103: Preparation of 6-(2,4-dichlorophenyl)-N-(1-(1-methylpiperidin-4-yl)-1H-pyrazol-4-yl)-8,9-dihydroimidazo[1′,2′:1,6]pyrido[2,3-d]pyrimidin-2-amine (Compound 103)

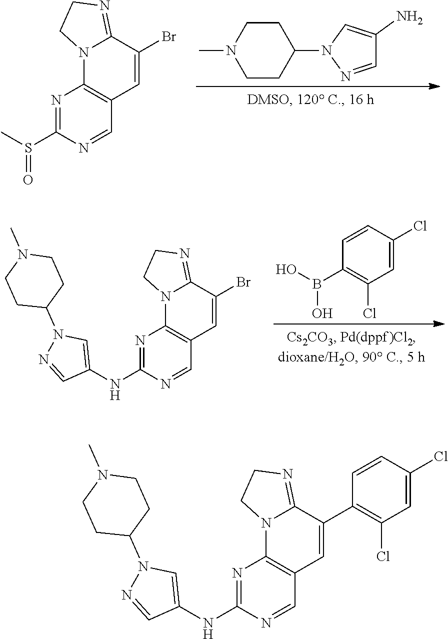

### Step 1: 6-bromo-N-(1-(1-methylpiperidin-4-yl)-1H-pyrazol-4-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (100 mg 0.32 mmol) and 1-(1-methylpiperidin-4-yl)-1H-pyrazol-4-amine (86 mg 0.48 mmol) in DMSO (3 mL) was stirred at 120° C. for 16 h. The reaction mixture was diluted with water (20 mL) and extracted with DCM (30 mL×3). The combined organic layers were washed with brine (20 mL), dried over Na₂SO₄, filtered, concentrated and the residue purified by column chromatography on silica gel (DCM/MeOH=8/1) to afford 6-bromo-N-(1-(1-methylpiperidin-4-yl)-1H-pyrazol-4-yl)-8,9-dihydroimidazo[1′,2′:1,6]pyrido[2,3-d]pyrimidin-2-amine (120 mg, 87.3% yield) as a brown solid. LCMS (M+H⁺) m/z: 429.1 and 431.1.

### Step 2: 6-(2,4-dichlorophenyl)-N-(1-(1-methylpiperidin-4-yl)-1H-pyrazol-4-yl)-8,9-dihydroimidazo[1′,2′:1,6]pyrido[2,3-d]pyrimidin-2-amine

To a solution of 6-bromo-N-(1-(1-methylpiperidin-4-yl)-1H-pyrazol-4-yl)-8,9-dihydroimidazo[1′,2′:1,6]pyrido[2,3-d]pyrimidin-2-amine (120 mg, 0.28 mmol) in dioxane/H₂O (10 mL/1 mL) was added (2,4-dichlorophenyl)boronic acid (59 mg, 0.31 mmol), Pd(dppf)Cl₂ (21 mg, 0.029 mmol), Cs₂CO₃ (283 mg, 0.84 mmol). The mixture was stirred for 4 h at 90° C. under N₂, concentrated and the residue was purified by Prep-HPLC (0.1% FA) and then by Prep-HPLC (0.1% NH₃·H₂O) to afford 6-(2,4-dichlorophenyl)-N-(1-(1-methylpiperidin-4-yl)-1H-pyrazol-4-yl)-8,9-dihydroimidazo[1′,2′:1,6]pyrido[2,3-d]pyrimidin-2-amine (7.5 mg, 5.4% yield) as a yellow solid. ¹H NMR (400 MHz, DMSO-d₆): δ 9.87-9.72 (m, 1H), 8.32 (s, 1H), 7.97-7.95 (m, 1H), 7.70 (s, 1H), 7.60-7.53 (m, 1H), 7.47 (s, 2H), 7.29 (s, 1H), 4.18-4.15 (m, 2H), 4.07-3.96 (m, 2H), 2.89-2.87 (m, 2H), 2.23 (s, 3H), 2.08-2.06 (m, 2H), 1.97-1.92 (m, 4H). LCMS (M+H⁺) m/z: 495.2.

### Example 104: Preparation of N-(6-(5-amino-2-bromo-4-fluorophenyl)-8,9-dihydroimidazo[1′,2′:1,6]pyrido[2,3-d]pyrimidin-2-yl)-N-methyl-4-(trifluoromethyl)picolinamide (Compound 104)

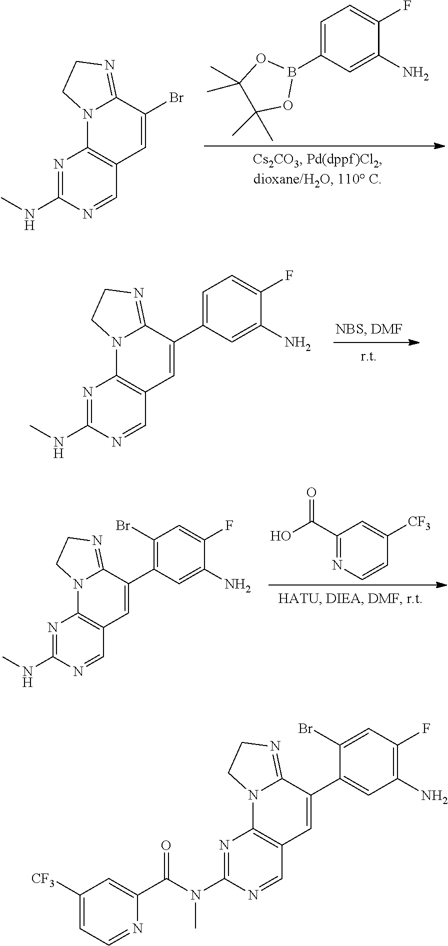

### Step 1: Synthesis of 6-(3-amino-4-fluorophenyl)-N-methyl-8,9-dihydroimidazo[1′,2′:1,6]pyrido[2,3-d]pyrimidin-2-amine

A mixture of 6-bromo-N-methyl-8,9-dihydroimidazo[1′,2′:1,6]pyrido[2,3-d]pyrimidin-2-amine (400 mg, 1.43 mmol), 2-fluoro-5-(4,4,5,5-tetramethyl-1,3,2-dioxaborolan-2-yl)aniline (510 mg, 2.1 mmol), Cs₂CO₃ (1.4 g, 4.29 mmol) and Pd(dppf)Cl₂ (100 mg, 0.143 mmol) in dioxane (15 mL) and water (3 mL) was stirred at 110° C. under N₂ for 18 h. The reaction mixture was cooled to r.t. and concentrated. The residue was purified by silica gel chromatography (DCM/MeOH=10/1) to afford 6-(3-amino-4-fluorophenyl)-N-methyl-8,9-dihydroimidazo[1′,2′:1,6]pyrido[2,3-d]pyrimidin-2-amine (248 mg, 56% yield) as a yellow solid. LCMS (M+H⁺) m/z: 311.3.

### Step 2: Synthesis of 6-(5-amino-2-bromo-4-fluorophenyl)-N-methyl-8,9-dihydroimidazo[1′,2′:1,6]pyrido[2,3-d]pyrimidin-2-amine

To a mixture of 6-(3-amino-4-fluorophenyl)-N-methyl-8,9-dihydroimidazo[1′,2′:1,6]pyrido[2,3-d]pyrimidin-2-amine (240 mg, 0.774 mmol) in DMF (5 mL) was added NBS (137 mg, 0.774 mmol). The solution was stirred at rt under N₂ for 2 h. Water was added, the mixture was extracted with EA twice. The combined extracts were concentrated and purified by flash chromatography to afford 6-(5-amino-2-bromo-4-fluorophenyl)-N-methyl-8,9-dihydroimidazo[1′,2′:1,6]pyrido[2,3-d]pyrimidin-2-amine (78 mg, 26% yield) as a yellow solid. LCMS (M+H⁺) m/z: 389.0, 391.0.

### Step 3: Synthesis of N-(6-(5-amino-2-bromo-4-fluorophenyl)-8,9-dihydroimidazo[1′,2′:1,6]pyrido[2,3-d]pyrimidin-2-yl)-N-methyl-4-(trifluoromethyl)picolinamide

A solution of 4-(trifluoromethyl)picolinic acid (22 mg, 0.116 mmol) and HATU (58 mg, 0.154 mmol) in DMF (3 mL) was stirred at rt for 15 mins, then the 6-(5-amino-2-bromo-4-fluorophenyl)-N-methyl-8,9-dihydroimidazo[1′,2′:1,6]pyrido[2,3-d]pyrimidin-2-amine (30 mg, 0.077 mmol) and DIEA (0.038 mL, 0.231 mmol) was added. The reaction was stirred at rt for 16 h. The solvent was removed and the residue was purified by Prep-HPLC (0.1% TFA) to afford N-(6-(5-amino-2-bromo-4-fluorophenyl)-8,9-dihydroimidazo[1′,2′:1,6]pyrido[2,3-d]pyrimidin-2-yl)-N-methyl-4-(trifluoromethyl)picolinamide as (5.4 mg, 12.5% yield) as a yellow solid. ¹H NMR (400 MHz, CD₃OD): δ 8.89 (s, 1H), 8.54 (d, J=5.2 Hz, 1H), 8.19 (s, 1H), 8.16 (s, 1H), 7.78 (d, J=5.2 Hz, 1H), 7.41 (d, J=10.4 Hz, 1H), 6.89 (d, J=8.8 Hz, 1H), 4.53-4.39 (m, 2H), 4.19-4.12 (m, 2H), 3.82 (s, 3H). LCMS (M+H⁺) m/z: 562.4.

### Example 105: Preparation of N-(4-methyl-3-(2-(methylamino)-9,10-dihydro-8H-pyrido[1,6-a:2,3-d′]dipyrimidin-6-yl)phenyl)-4-(trifluoromethyl)picolinamide (Compound 105)

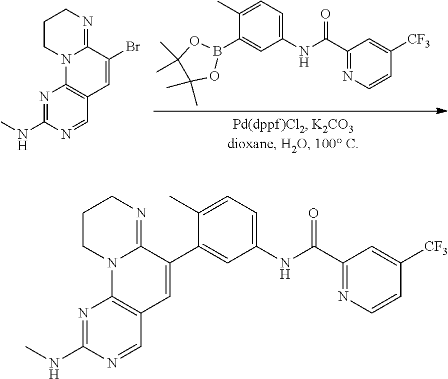

The preparation of 6-bromo-N-methyl-9,10-dihydro-8H-pyrido[1,6-a:2,3-d′]dipyrimidin-2-amine and N-(4-methyl-3-(4,4,5,5-tetramethyl-1,3,2-dioxaborolan-2-yl)phenyl)-4-(trifluoromethyl)picolinamide was described in Example 76 and Example 74.

### Step 1: Synthesis of N-(4-methyl-3-(2-(methylamino)-9,10-dihydro-8H-pyrido[1,6-a:2,3-d′]dipyrimidin-6-yl)phenyl)-4-(trifluoromethyl)picolinamide

The mixture of 6-bromo-N-methyl-9,10-dihydro-8H-pyrido[1,6-a:2,3-d′]dipyrimidin-2-amine (100 mg, 0.34 mmol, 1.0 eq), N-(4-methyl-3-(4,4,5,5-tetramethyl-1,3,2-dioxaborolan-2-yl)phenyl)-4-(trifluoromethyl)picolinamide (207 mg, 0.51 mmol, 1.5 eq), K₂CO₃ (141 mg, 1.02 mmol, 3.0 eq) and Pd(dppf)Cl₂ (10 mg) in dioxane (20 mL) and H₂O (2 mL) was stirred at 100° C. for 2 h. The mixture was purified by Prep-HPLC (0.1% FA) and (0.1% NH₄HCO₃) to afford N-(4-methyl-3-(2-(methylamino)-9,10-dihydro-8H-pyrido[1,6-a:2,3-d′]dipyrimidin-6-yl)phenyl)-4-(trifluoromethyl)picolinamide (25.9 mg, 15.9% yield) as a yellow solid. ¹H NMR (400 MHz, DMSO-d₆): δ 10.70 (s, 1H), 9.03-9.01 (m, 1H), 8.33 (s, 1H), 8.25 (s, 1H), 8.08 (d, J=5.2 Hz, 1H), 7.74-7.72 (m, 2H), 7.40-7.29 (m, 1H), 7.18 (d, J=8.8 Hz, 1H), 7.03 (s, 1H), 4.19-4.02 (m, 2H), 3.39 (s, 2H), 2.87 (d, J=4.4 Hz, 3H), 2.13 (s, 3H), 1.86 (s, 2H). LCMS (M+H⁺) m/z: 494.0.

### Example 106: Preparation of N-(3-(2-amino-8,9-dihydroimidazo[1′,2′:1,6]pyrido[2,3-d]pyrimidin-6-yl)-4-methylphenyl)-4-(trifluoromethyl)picolinamide (compound 106)

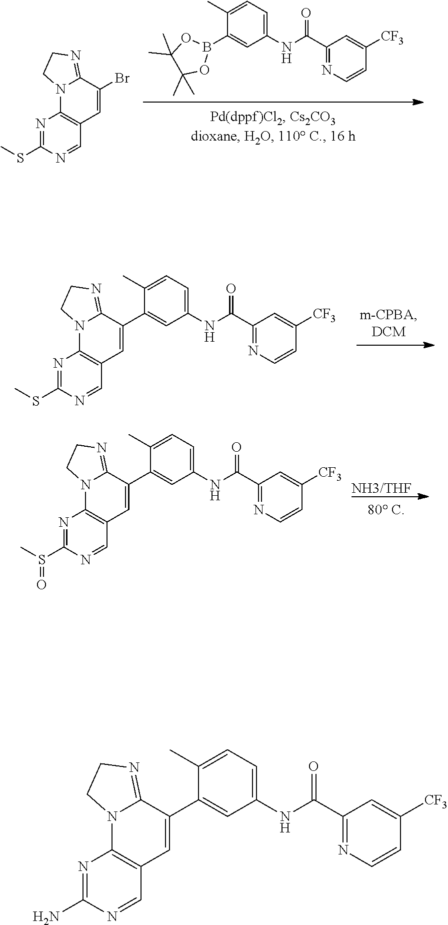 [SMILES: CSc1ncc2c(n1)N1CCN=C1C(Br)=C2 ; Cc1ccc(NC(=O)c2cc(C(F)(F)F)ccn2)cc1B1OC(C)(C)C(C)(C)O1 ; CSc1ncc2c(n1)N1CCN=C1C(c1cc(NC(=O)c3cc(C(F)(F)F)ccn3)ccc1C)=C2 ; Cc1ccc(NC(=O)c2cc(C(F)(F)F)ccn2)cc1C1=Cc2cnc(S(C)=O)nc2N2CCN=C12 ; Cc1ccc(NC(=O)c2cc(C(F)(F)F)ccn2)cc1C1=Cc2cnc(N)nc2N2CCN=C12]

The preparation of N-(4-methyl-3-(4,4,5,5-tetramethyl-1,3,2-dioxaborolan-2-yl)phenyl)-4-(trifluoromethyl)picolinamide was described in Example 76. The preparation of 6-bromo-2-(methylthio)-8,9-dihydroimidazo[1′,2′:1,6]pyrido[2,3-d]pyrimidine was described in Example 26.

### Step 1: Synthesis of N-(4-methyl-3-(2-(methylthio)-8,9-dihydroimidazo[1′,2′:1,6]pyrido[2,3-d]pyrimidin-6-yl)phenyl)-4-(trifluoromethyl)picolinamide

A mixture of 6-bromo-2-(methylthio)-8,9-dihydroimidazo[1′,2′:1,6]pyrido[2,3-d]pyrimidine (1.83 g, 6.16 mmol), N-(4-methyl-3-(4,4,5,5-tetramethyl-1,3,2-dioxaborolan-2-yl)phenyl)-4-(trifluoromethyl) picolinamide (3.0 g, 7.39 mmol), Cs₂CO₃ (6.0 g, 18.48 mmol) and Pd(dppf)Cl₂ (316 mg, 0.43 mmol) in dioxane (40.0 mL) and water (4.0 mL) was degassed and charged with N₂ for three times and stirred at 110° C. for 16 h. The reaction mixture was concentrated and purified by column chromatography (PE/EA=1/2, +0.1% TEA) to afford N-(4-methyl-3-(2-(methylthio)-8,9-dihydroimidazo[1′,2′:1,6]pyrido[2,3-d]pyrimidin-6-yl)phenyl)-4-(trifluoromethyl)picolinamide (2.67 g, 87% yield) as a yellow solid. LCMS (M+H⁺) m/z: 497.1.

### Step 2: Synthesis of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide

To a solution of N-(4-methyl-3-(2-(methylthio)-8,9-dihydroimidazo[1′,2′:1,6]pyrido[2,3-d]pyrimidin-6-yl)phenyl)-4-(trifluoromethyl)picolinamide (300 mg, 0.6 mmol) in dry DCM (8.0 mL) was added m-CPBA (347 mg, 1.51 mmol) at 0° C. The resulting mixture was stirred at 0° C. for 30 min. The reaction mixture was concentrated at r.t. under vacuum to afford crude N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (650 mg, 100% yield) as a yellow solid which was used in the next step without purification. LCMS (M+H⁺) m/z: 513.1.

### Step 3: Synthesis of N-(3-(2-amino-8,9-dihydroimidazo[1′,2′:1,6]pyrido[2,3-d]pyrimidin-6-yl)-4-methylphenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (170 mg, 0.33 mmol, 1.0 eq) and NH₃-THF (10 mL) was stirred at 80° C. for 16 h, the reaction was monitored by LCMS, The mixture was purified by Prep-HPLC (0.1% NH₄HCO₃) and (0.1% FA) to afford product (26.5 mg, 17.2% yield) as a yellow solid. ¹H NMR (400 MHz, DMSO-d₆): δ 10.74 (s, 1H), 9.03 (d, J=4.8 Hz, 1H), 8.33 (s, 1H), 8.20 (s, 1H), 8.19 (s, 1H), 8.09-8.08 (m, 1H), 7.81 (d, J=2.4 Hz, 1H), 7.78-7.75 (m, 1H), 7.23 (d, J=8.4 Hz, 1H), 7.15 (s, 1H), 7.00 (s, 2H), 4.02-4.00 (m, 2H), 3.93-3.91 (m, 2H), 2.20 (s, 3H). LCMS (M+H⁺) m/z: 466.0.

### Example 107: Preparation of N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (compound 107)

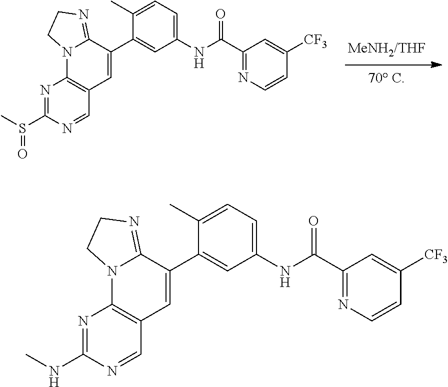 [SMILES: Cc1ccc(NC(=O)c2cc(C(F)(F)F)ccn2)cc1C1=Cc2cnc(S(C)=O)nc2N2CCN=C12 ; CNc1ncc2c(n1)N1CCN=C1C(c1cc(NC(=O)c3cc(C(F)(F)F)ccn3)ccc1C)=C2]

### Step 1: Synthesis of N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (170 mg, 0.33 mmol, 1.0 eq) and MeNH₂-THF (10 mL) was stirred at 70° C. for 1 h, the reaction was monitored by LCMS. The mixture was purified by Prep-HPLC (0.1% NH4HCO3) to afford N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (22 mg, 13.8% yield) as a yellow solid.

### Example 108: Preparation of N-(3-(2-(dimethylamino)-8,9-dihydroimidazo[1′,2′:1,6]pyrido[2,3-d]pyrimidin-6-yl)-4-methylphenyl)-4-(trifluoromethyl)picolinamide

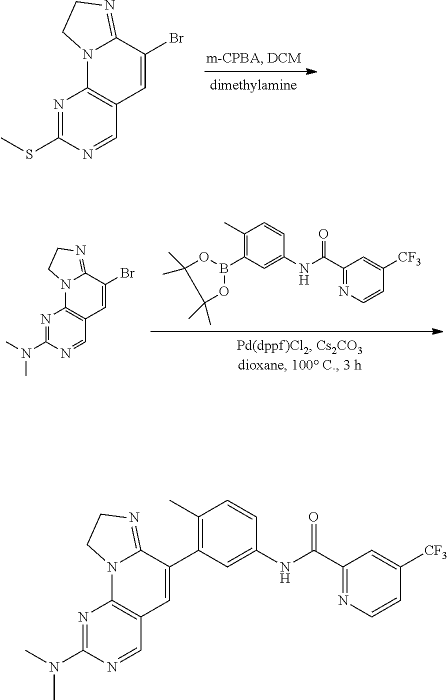

### Step 1: Synthesis of 6-bromo-N,N-dimethyl-8,9-dihydroimidazo[1′,2′:1,6]pyrido[2,3-d]pyrimidin-2-amine

To a solution of 6-bromo-2-(methylthio)-8,9-dihydroimidazo[1′,2′:1,6]pyrido[2,3-d]pyrimidine (500 mg, 1.682 mmol) in DCM (10 mL) was added m-CPBA (871 mg, 5.047 mmol). The reaction mixture was stirred at R.T under N₂ for 1 h. Then dimethylamine (6.0 ml) was added to above reaction solution. The reaction mixture was stirred at R.T under N₂ for 16 h. The result solution was washed with NH₄Cl, concentrated. The residue was purified by silica gel chromatography (DCM:MeOH=30:1) to get 6-bromo-N,N-dimethyl-8,9-dihydroimidazo[1′,2′:1,6]pyrido[2,3-d]pyrimidin-2-amine (360 mg, 72.7%) as a yellow solid. LCMS (M+H⁺) m/z: 296.1.

### Step 2: Synthesis of N-(3-(2-(dimethylamino)-8,9-dihydroimidazo[1′,2′:1,6]pyrido[2,3-d]pyrimidin-6-yl)-4-methylphenyl)-4-(trifluoromethyl)picolinamide

To a solution of 6-bromo-N,N-dimethyl-8,9-dihydroimidazo[1′,2′:1,6]pyrido[2,3-d]pyrimidin-2-amine (100 mg, 0.339 mmol), N-(4-methyl-3-(4,4,5,5-tetramethyl-1,3,2-dioxaborolan-2-yl)phenyl)-4-(trifluoromethyl)picolinamide (138 mg, 0.339 mmol) and Cs₂CO₃ (332 mg, 1.019 mmol) in dioxane (8 mL) and H₂O (2 mL) was added Pd(dppf)Cl₂ (25 mg, 0.034 mmol). The reaction mixture was stirred at 100° C. under N₂ for 3 h. The result solution was extracted with EA (20 mL×3), concentrated. The crude product was purified by prep-TLC (DCM:MeOH=30:1) and further by prep-HPLC (0.1%/FA/CH₃CN/H₂O) to afford N-(3-(2-(dimethylamino)-8,9-dihydroimidazo[1′,2′:1,6]pyrido[2,3-d]pyrimidin-6-yl)-4-methylphenyl)-4-(trifluoromethyl)picolinamide (48.3 mg, 28.8% yield) as a yellow solid. ¹H NMR (400 MHz, DMSO-d₆) δ 10.76 (s, 1H), 9.03 (d, J=5.2 Hz, 1H), 8.33 (s, 1H), 8.26 (s, 1H), 8.08 (d, J=4.8 Hz, 2H), 7.82 (d, J=2 Hz, 1H), 7.76 (d, J=8.4 Hz, 1H), 7.22 (d, J=8.4 Hz, 1H), 7.14 (s, 1H), 4.07-4.02 (m, 2H), 3.94-3.89 (m, 2H), 3.18 (s, 6H), 2.20 (s, 3H); LCMS (M+H⁺) m/z: 496.9.

### Example 109: Preparation of N-(3-(2-(ethylamino)-8,9-dihydroimidazo[1′,2′:1,6]pyrido[2,3-d]pyrimidin-6-yl)-4-methylphenyl)-4-(trifluoromethyl)picolinamide formate (Compound 109)

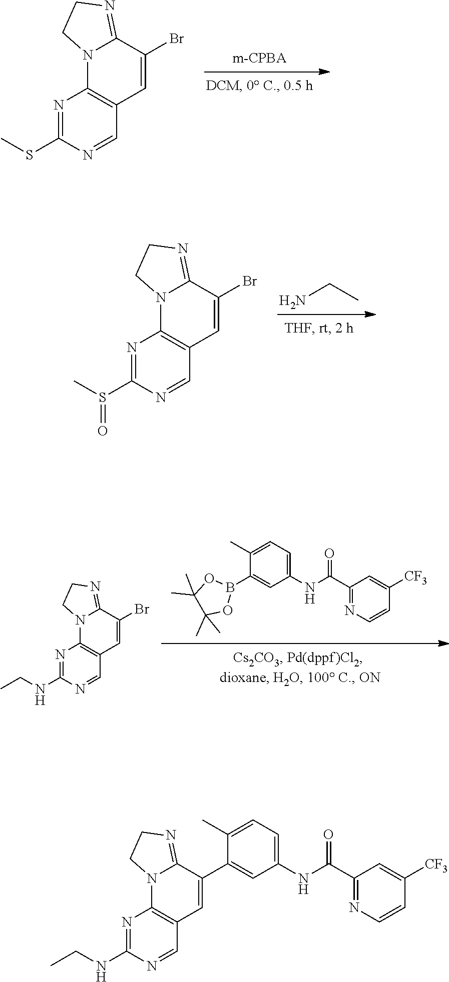

The preparation of 6-bromo-2-(methylthio)-8,9-dihydroimidazo[1′,2′:1,6]pyrido[2,3-d]pyrimidine was described in Example 26.

### Step 1: Synthesis of 6-bromo-2-(methylsulfinyl)-8,9-dihydroimidazo[1′,2′:1,6]pyrido[2,3-d]pyrimidine

A mixture of 6-bromo-2-(methylthio)-8,9-dihydroimidazo[1′,2′:1,6]pyrido[2,3-d]pyrimidine (1.0 g, 3.38 mmol) in dry DCM (20 mL) was added m-CPBA (1.0 g, 5.07 mmol, 70% wt) at 0° C. The resulting mixture was stirred at 0° C. for 30 min. The reaction mixture was diluted with sat. NaHCO3 (aq.) (20 mL) and extracted with DCM (20 mL×2). The combined organic phase was washed with brine (20 mL), dried with Na₂SO₄, filtered and concentrated in vacuum to afford crude 6-bromo-2-(methylsulfinyl)-8,9-dihydroimidazo[1′,2′:1,6]pyrido[2,3-d]pyrimidine (1.2 g, crude) as a yellow solid, which was used in the next step without purification. LCMS (M+H⁺) m/z: 312.9 and 314.9.

### Step 2: Synthesis of 6-bromo-N-ethyl-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (crude, 765 mg, 1.01 mmol) in dry-THF (2.5 mL) was added ethanamine (0.8 mL, 1.56 mmol, 2 M in THF). The mixture was stirred at rt for 2 h. The mixture was diluted with water (50.0 mL), extracted with EA (50 mL×3). The combined organic phase was washed with brine (50 mL×3), dried with Na₂SO₄, filtered and concentrated. The residue was triturated with EA (5.0 mL) to afford 6-bromo-N-ethyl-8,9-dihydroimidazo[1′,2′:1,6]pyrido[2,3-d]pyrimidin-2-amine (290 mg, 97% yield) as a yellow solid. LCMS (M+H⁺) m/z: 294.0 and 296.0.

### Step 3: Synthesis of N-(3-(2-(ethylamino)-8,9-dihydroimidazo[1′,2′:1,6]pyrido[2,3-d]pyrimidin-6-yl)-4-methylphenyl)-4-(trifluoromethyl)picolinamide formate

A mixture of 6-bromo-N-ethyl-8,9-dihydroimidazo[1′,2′:1,6]pyrido[2,3-d]pyrimidin-2-amine (150 mg, 0.51 mmol), N-(4-methyl-3-(4,4,5,5-tetramethyl-1,3,2-dioxaborolan-2-yl)phenyl)-4-(trifluoromethyl)picolinamide (228 mg, 0.56 mmol), Cs₂CO₃ (500 mg, 1.54 mmol) and Pd(dppf)Cl₂ (25 mg, 0.03 mmol) in dioxane (10 mL) and water (1 mL) was degassed and charged with N₂ three times, then stirred at 100° C. for 16 h. The reaction mixture was concentrated and purified by Prep-HPLC (0.1% NH₃—H₂O) to afford N-(3-(2-(ethylamino)-8,9-dihydroimidazo[1′,2′:1,6]pyrido[2,3-d]pyrimidin-6-yl)-4-methylphenyl)-4-(trifluoromethyl)picolinamide formate (20.8 mg, 8.2% yield) as a yellow solid. ¹H NMR (400 MHz, DMSO-d₆): δ 10.74 (s, 1H), 9.03 (d, J=4.8 Hz, 1H), 8.33 (s, 1H), 8.22 (s, 1H), 8.09-8.08 (m, 1H), 7.81 (d, J=2.0 Hz, 1H), 7.78 (dd, J=8.4, 2.4 Hz, 1H), 7.59-7.43 (m, 1H), 7.23 (d, J=8.4 Hz, 1H), 7.14 (s, 1H), 4.04-4.01 (m, 2H), 3.97-3.88 (m, 2H), 3.38-3.29 (m, 2H), 2.20 (s, 3H), 1.14 (s, 3H). LCMS (M+H⁺) m/z: 494.3.

### Example 111: Preparation of N-(3-(2-((2-fluoroethyl)amino)-8,9-dihydroimidazo[1′,2′:1,6]pyrido[2,3-d]pyrimidin-6-yl)-4-methylphenyl)-4-(trifluoromethyl)picolinamide (compound 111)

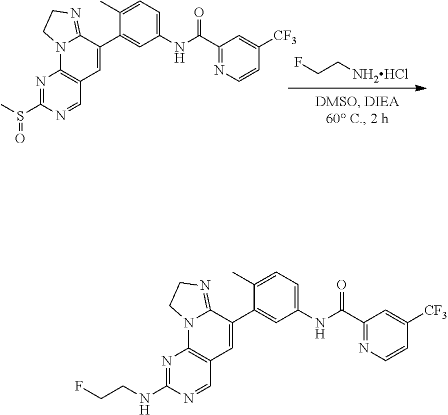

The preparation of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide was described in Example 106.

### Step 1: Synthesis of N-(3-(2-((2-fluoroethyl)amino)-8,9-dihydroimidazo[1′,2′:1,6]pyrido[2,3-d]pyrimidin-6-yl)-4-methylphenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (120 mg, crude), DIEA (0.3 mL) and 2-fluoroethan-1-amine hydrochloride (100 mg) in DMSO (1 mL) was stirred at 60° C. for 2 h. The reaction was monitored by LCMS. The mixture was purified by Prep-HPLC (0.1% FA) to afford product (19.4 mg) as a yellow solid. ¹H NMR (400 MHz, DMSO-d6) ppm: δ 10.79 (s, 1H), 9.03 (d, J=5.6 Hz, 1H), 8.39-8.33 (m, 1H), 8.14 (s, 1H), 8.10-8.09 (m, 1H), 8.01-7.79 (m, 3H), 7.41-7.26 (m, 2H), 4.66-4.49 (m, 2H), 4.20-4.09 (m, 2H), 4.07-4.91 (m, 2H), 3.70-3.60 (m, 2H), 2.20 (s, 3H). LCMS (M+H⁺) m/z: 511.9.

### Example 112: Preparation of N-(3-(2-((2-methoxyethyl)amino)-8,9-dihydroimidazo[1′,2′:1,6]pyrido[2,3-d]pyrimidin-6-yl)-4-methylphenyl)-4-(trifluoromethyl)picolinamide (compound 112)

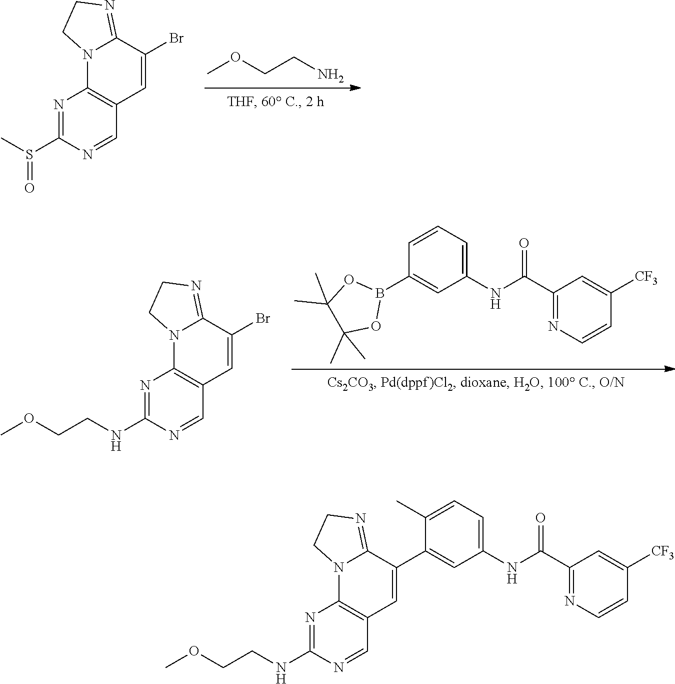

### Step 1: Synthesis of 6-bromo-N-(2-methoxyethyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (crude, 765 mg, 1.01 mmol) in dry-THF (2.5 mL) was added 2-methoxyethan-1-amine (117 mg, 1.56 mmol). The mixture was stirred at 60° C. for 2 h. The reaction was cooled to r.t. and diluted with water (50.0 mL), extracted with EA (50 mL×3). The combined organic phase was washed with brine (50 mL), dried with Na₂SO₄, filtered and concentrated. The residue was triturated with EA (5.0 mL) to afford 6-bromo-N-(2-methoxyethyl)-8,9-dihydroimidazo[1′,2′:1,6]pyrido[2,3-d]pyrimidin-2-amine (200 mg, 61% yield) as a yellow solid. LCMS (M+H⁺) m/z:324.1 and 326.1.

### Step 2: Synthesis of N-(3-(2-((2-methoxyethyl)amino)-8,9-dihydroimidazo[1′,2′:1,6]pyrido[2,3-d]pyrimidin-6-yl)-4-methylphenyl)-4-(trifluoromethyl)picolinamide

A mixture of 6-bromo-N-(2-methoxyethyl)-8,9-dihydroimidazo[1′,2′:1,6]pyrido[2,3-d]pyrimidin-2-amine (200 mg, 0.62 mmol), N-(4-methyl-3-(4,4,5,5-tetramethyl-1,3,2-dioxaborolan-2-yl)phenyl)-4-(trifluoromethyl)picolinamide (275 mg, 0.68 mmol), Cs₂CO₃ (600 mg, 1.85 mmol) and Pd(dppf)Cl₂ (25 mg, 0.03 mmol) in dioxane (10 mL) and water (1 mL) was degassed and charged with N₂ three times, stirred at 100° C. for 16 h. The reaction mixture was concentrated and purified by Prep-HPLC (0.1% NH₃·H₂O) to afford N-(3-(2-((2-methoxyethyl)amino)-8,9-dihydroimidazo[1′,2′:1,6]pyrido[2,3-d]pyrimidin-6-yl)-4-methylphenyl)-4-(trifluoromethyl)picolinamide (57.9 mg, 18% yield) as a yellow solid. ¹H NMR (400 MHz, DMSO-d₆): δ 10.76 (s, 1H), 9.03 (d, J=4.8 Hz, 1H), 8.33 (s, 1H), 8.27 (s, 1H), 8.17 (s, 1H), 8.09 (dd, J=0.8 Hz, 3.6 Hz, 1H), 7.83 (d, J=2.0 Hz, 1H), 7.79 (dd, J=2.0 Hz, 8.4 Hz, 1H), 7.58-7.47 (m, 1H), 7.25-7.23 (m, 1H), 4.09-4.08 (m, 1H), 3.94-3.89 (m, 1H), 3.50-3.49 (m, 3H), 3.28 (s, 3H), 2.20 (s, 3H). LCMS (M+H⁺) m/z: 524.4.

### Example 113: Preparation of N-(3-(2-((2-hydroxy-2-methylpropyl)amino)-8,9-dihydroimidazo[1′,2′:1,6]pyrido[2,3-d]pyrimidin-6-yl)-4-methylphenyl)-4-(trifluoromethyl)picolinamide (Compound 113)

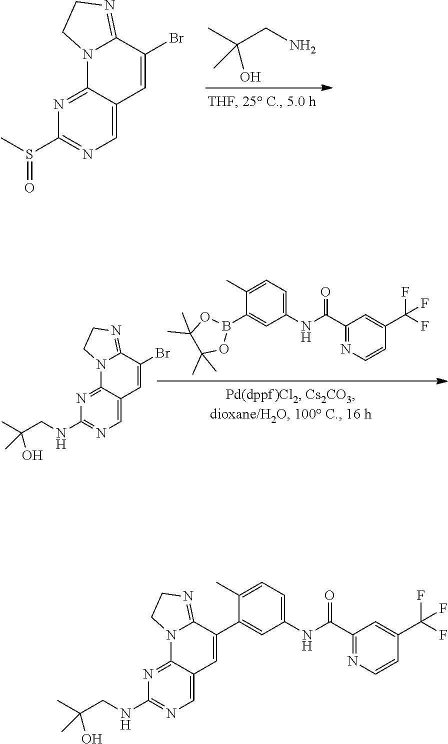

### Step 1: Synthesis of 1-((6-bromo-8,9-dihydroimidazo[1′,2′:1,6]pyrido[2,3-d]pyrimidin-2-yl)amino)-2-methylpropan-2-ol

To a mixture of 6-bromo-2-(methylsulfinyl)-8,9-dihydroimidazo[1′,2′:1,6]pyrido[2,3-d]pyrimidine (205 mg, 0.65 mmol) in dry-THF (5.0 mL) was added 1-amino-2-methylpropan-2-ol (175 mg, 1.96 mmol). The resulting mixture was stirred at r.t. for 5 h. The reaction mixture was concentrated and diluted with EA (3.0 mL), stirred at r.t. for 1 h, filtered. The collected cake was dried to afford 1-((6-bromo-8,9-dihydroimidazo[1′,2′:1,6]pyrido[2,3-d]pyrimidin-2-yl)amino)-2-methylpropan-2-ol (115 mg, 52.3% yield) as a yellow solid. LCMS (M+H⁺) m/z: 337.9 and 339.9.

### Step 2: Synthesis of N-(3-(2-((2-hydroxy-2-methylpropyl)amino)-8,9-dihydroimidazo[1′,2′:1,6]pyrido[2,3-d]pyrimidin-6-yl)-4-methylphenyl)-4-(trifluoromethyl)picolinamide

A mixture of 1-((6-bromo-8,9-dihydroimidazo[1′,2′:1,6]pyrido[2,3-d]pyrimidin-2-yl)amino)-2-methylpropan-2-ol (115 mg, 0.34 mmol), N-(4-methyl-3-(4,4,5,5-tetramethyl-1,3,2-dioxaborolan-2-yl)phenyl)-4-(trifluoromethyl)picolinamide (160 mg, 0.39 mmol), Cs₂CO₃ (277 mg, 0.85 mmol) and Pd(dppf)Cl₂ (25 mg, 0.034 mmol) in dioxane (5.0 mL) and water (0.5 mL) was degassed and charged with N₂ three times and stirred at 100° C. for 16 h. The reaction mixture was concentrated, diluted with water (30.0 mL), extracted with DCM (30.0 mL×3). The combined organic phase was washed with brine, dried over Na₂SO₄, filtered and concentrated. The residue was purified by silica column (DCM:MeOH=20:1, +0.1% NH₃/MeOH) to afford N-(3-(2-((2-hydroxy-2-methylpropyl)amino)-8,9-dihydroimidazo[1′,2′:1,6]pyrido[2,3-d]pyrimidin-6-yl)-4-methylphenyl)-4-(trifluoromethyl)picolinamide (40 mg, 22% yield) as a yellow solid. ¹H NMR (400 MHz, DMSO-d₆): δ10.75 (s, 1H), 9.03 (d, J=5.2 Hz, 1H), 8.33 (s, 1H), 8.23 (s, 1H), 8.08 (d, J=4.4 Hz, 1H), 7.81 (d, J=1.6 Hz, 1H), 7.77 (dd, J=2.0 Hz, 8.0 Hz, 1H), 7.24-7.15 (m, 3H), 4.63-4.57 (m, 1H), 4.05-4.00 (m, 2H), 3.92 (t, J=8.8 Hz, 2H), 3.35 (d, J=6.4 Hz, 2H), 2.20 (s, 3H), 1.19-1.12 (m, 5H). LCMS (M+H⁺) m/z: 538.7.

### Example 114: Preparation of N-(4-methyl-3-(2-(oxetan-3-ylamino)-8,9-dihydroimidazo[1′,2′:1,6]pyrido[2,3-d]pyrimidin-6-yl)phenyl)-4-(trifluoromethyl)picolinamide (Compound 114)

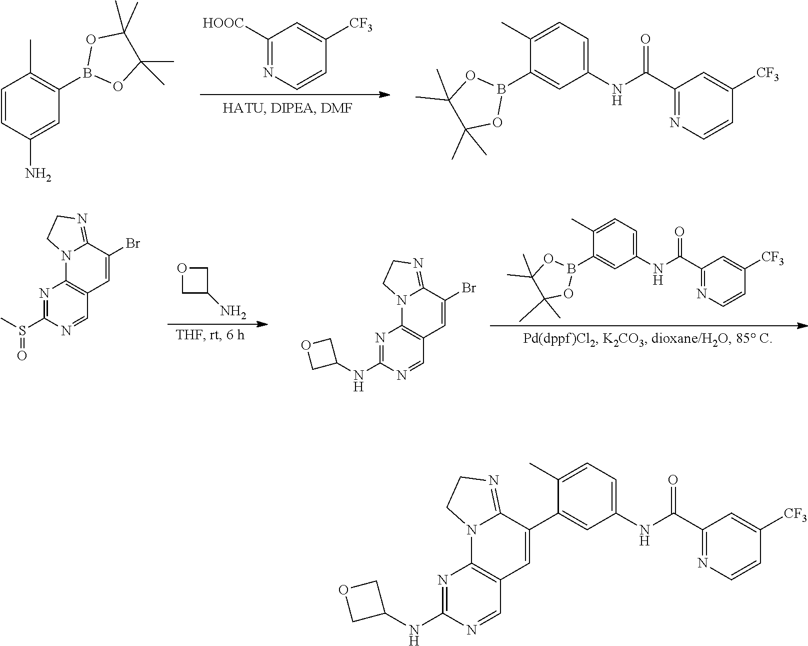

The preparation of 6-bromo-2-(methylsulfinyl)-8,9-dihydroimidazo[1′,2′:1,6]pyrido[2,3-d]pyrimidine was described and in Example 26.

### Step 1: Synthesis of 6-bromo-N-(oxetan-3-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (1.9 g, 6.07 mmol), oxetan-3-amine (2.2 g, 30.35 mmol), DIEA (1.57 g, 12.14 mmol) in THF (40.0 mL) was stirred at 30° C. for 16 h. The reaction mixture was concentrated. The residue was triturated with EA (10.0 mL) to afford crude 6-bromo-N-(oxetan-3-yl)-8,9-dihydroimidazo[1′,2′:1,6]pyrido[2,3-d]pyrimidin-2-amine (2.0 g, 100% yield) as a yellow solid, which was used in the next step without purification. LCMS (M+H⁺) m/z: 321.9 and 323.9.

### Step 2: N-(4-methyl-3-(4,4,5,5-tetramethyl-1,3,2-dioxaborolan-2-yl)phenyl)-4-(trifluoromethyl) picolinamide

To a solution of 4-(trifluoromethyl)picolinic acid (769 mg, 4.0 mmol) in DMF (25 mL) was added 4-methyl-3-(4,4,5,5-tetramethyl-1,3,2-dioxaborolan-2-yl)aniline (985 mg, 4.22 mmol), HATU (2.28 g, 6.0 mmol), DIPEA (1.5 g, 12.0 mmol). The mixture was stirred at rt for 1 h. H₂O (100 mL) was added to the reaction mixture. The resulting mixture was filtered and the cake was washed with H₂O (20 mL×3). The solid was dried to afford N-(4-methyl-3-(4,4,5,5-tetramethyl-1,3,2-dioxaborolan-2-yl)phenyl)-4-(trifluoromethyl) picolinamide (1.76 g, 96% yield) as a light grey solid. LCMS (M+H⁺) m/z: 407.1.

### Step 3: N-(4-methyl-3-(2-(oxetan-3-ylamino)-8,9-dihydroimidazo[1′,2′:1,6]pyrido[2,3-d]pyrimidin-6-yl)phenyl)-4-(trifluoromethyl)picolinamide

To a solution of 6-bromo-N-(oxetan-3-yl)-8,9-dihydroimidazo[1′,2′:1,6]pyrido[2,3-d]pyrimidin-2-amine (231 mg, 0.72 mmol) in dioxane/H₂O (20 mL/4 mL) was added N-(4-methyl-3-(4,4,5,5-tetramethyl-1,3,2-dioxaborolan-2-yl)phenyl)-4-(trifluoromethyl)picolinamide (306 mg, 0.75 mmol), Pd(dppf)Cl₂ (79 mg, 0.108 mmol), K₂CO₃ (298 mg, 2.16 mmol). The mixture was stirred for 16 h at 85° C. under N₂. H₂O (100 mL) was added to the reaction mixture. The mixture was extracted with EA (30 mL×3). The combined organic layers was washed with brine (20 mL×2), dried over Na₂SO₄, concentrated under vacuum and purified by Prep-HPLC (1% HCOOH) to afford N-(4-methyl-3-(2-(oxetan-3-ylamino)-8,9-dihydroimidazo[1′,2′:1,6]pyrido[2,3-d]pyrimidin-6-yl)phenyl)-4-(trifluoromethyl)picolinamide (59.1 mg, 16% yield) as a yellow solid. ¹H NMR (400 MHz, DMSO-d₆): δ 10.75 (s, 1H), 9.03 (d, J=4.8 Hz, 1H), 8.33 (s, 1H), 8.25 (s, 2H), 8.09-8.08 (m, 1H), 7.82 (d, J=2.0 Hz, 1H), 7.78-7.76 (m, 1H), 7.23 (d, J=8.4 Hz, 1H), 7.17-7.17 (m, 1H), 4.96-4.95 (m, 1H), 4.79-4.77 (m, 2H), 4.55 (t, J=6.0 Hz, 2H), 4.06-4.01 (m, 2H), 3.94-3.89 (m, 2H), 2.20 (s, 3H). LCMS (M+H⁺) m/z: 522.4.

### Example 115: Preparation of N-(4-methyl-3-(2-((tetrahydro-2H-pyran-4-yl)amino)-8,9-dihydroimidazo[1′,2′:1,6]pyrido[2,3-d]pyrimidin-6-yl)phenyl)-4-(trifluoromethyl)picolinamide hydrogen chloride (Compound 115)

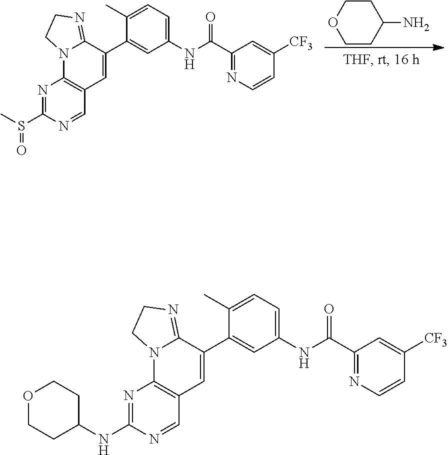

The preparation of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide was described in Example 106.

### Step 1: Synthesis of N-(4-methyl-3-(2-((tetrahydro-2H-pyran-4-yl)amino)-8,9-dihydroimidazo[1′,2′:1,6]pyrido[2,3-d]pyrimidin-6-yl)phenyl)-4-(trifluoromethyl)picolinamide hydrogen chloride

To a solution of crude N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (650 mg, 0.60 mmol) in THF (8.0 mL) was added tetrahydro-2H-pyran-4-amine (305 mg, 3.0 mmol). The mixture was stirred at r.t. for 16 h, concentrated and added with water (80.0 mL), extracted with EA (50 mL×3). The combined organic phase was washed with brine (100 mL), dried with Na₂SO₄, filtered and concentrated. The residue was purified by Prep-HPLC (0.1% HCl) to afford N-(4-methyl-3-(2-((tetrahydro-2H-pyran-4-yl)amino)-8,9-dihydroimidazo[1′,2′:1,6]pyrido[2,3-d]pyrimidin-6-yl)phenyl)-4-(trifluoromethyl)picolinamide hydrogen chloride (11.0 mg, 3.1% yield) as a yellow solid. ¹H NMR (400 MHz, DMSO-d₆): δ 10.92 (s, 1H), 9.84 (s, 0.6H), 9.76 (s, 0.4H), 9.06-8.91 (m, 1H), 8.66-8.52 (m, 1H), 8.34 (s, 1H), 8.14-8.11 (m, 2H), 7.99 (s, 1H), 7.91 (d, J=7.2 Hz, 1H), 7.41 (d, J=7.6 Hz, 1H), 4.67-4.58 (m, 2H), 4.05-3.89 (m, 6H), 3.19-3.18 (m, 1H), 2.21 (s, 3H), 1.95-1.82 (m, 2H), 1.63-1.60 (m, 2H). LCMS (M+H⁺) m/z: 550.4.

### Example 116: Preparation of N-(3-(2-(azetidin-3-ylamino)-8,9-dihydroimidazo[1′,2′:1,6]pyrido[2,3-d]pyrimidin-6-yl)-4-methylphenyl)-4-(trifluoromethyl)picolinamide (Compound 116)

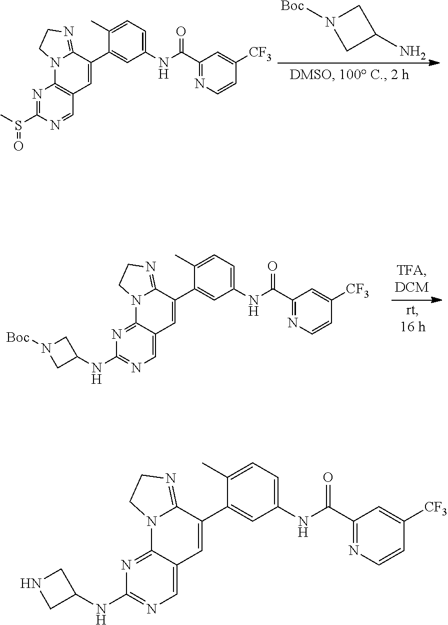 [SMILES: Cc1ccc(NC(=O)c2cc(C(F)(F)F)ccn2)cc1C1=Cc2cnc(S(C)=O)nc2N2CCN=C12 ; CC(C)(C)OC(=O)N1CC(N)C1 ; Cc1ccc(NC(=O)c2cc(C(F)(F)F)ccn2)cc1C1=Cc2cnc(NC3CN(C(=O)OC(C)(C)C)C3)nc2N2CCN=C12 ; Cc1ccc(NC(=O)c2cc(C(F)(F)F)ccn2)cc1C1=Cc2cnc(NC3CNC3)nc2N2CCN=C12]

### Step 1: Synthesis of tert-butyl 3-((6-(2-methyl-5-(4-(trifluoromethyl)picolinamido)phenyl)-8,9-dihydroimidazo[1′,2′:1,6]pyrido[2,3-d]pyrimidin-2-yl)amino)azetidine-1-carboxylate

To a solution of crude N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (89 mg, 0.15 mmol) in DMSO (8.0 mL) was added tert-butyl 3-aminoazetidine-1-carboxylate (86 mg, 0.5 mmol). The mixture was stirred at 120° C. for 2 h. The reaction mixture was diluted with water (80.0 mL), extracted with EA (50 mL×3). The combined organic phase was washed with brine (100 mL), dried with Na₂SO₄, filtered and concentrated. The residue was purified by flash (DCM:MeOH=20:1, +0.1% NH₃/MeOH) to afford (60 mg, 67% yield) as a yellow solid.

### Step 2: Synthesis of N-(3-(2-(azetidin-3-ylamino)-8,9-dihydroimidazo[1′,2′:1,6]pyrido[2,3-d]pyrimidin-6-yl)-4-methylphenyl)-4-(trifluoromethyl)picolinamide

A mixture of tert-butyl 3-((6-(2-methyl-5-(4-(trifluoromethyl)picolinamido)phenyl)-8,9-dihydroimidazo[1′,2′:1,6]pyrido[2,3-d]pyrimidin-2-yl)amino)azetidine-1-carboxylate (60 mg, 0.1 mmol) and TFA (1 mL) in DCM (10 mL) was stirred at rt overnight. The resulting mixture was evaporated and the residue was purified by prep-HPLC (0.1% NH₃H₂O) to afford N-(3-(2-(azetidin-3-ylamino)-8,9-dihydroimidazo[1′,2′:1,6]pyrido[2,3-d]pyrimidin-6-yl)-4-methylphenyl)-4-(trifluoromethyl)picolinamide (12 mg, 30% yield) as a white solid. ¹H NMR (400 MHz, CD₃OD): δ 8.95 (d, J=5.2 Hz, 1H), 8.40 (s, 1H), 8.24 (s, 1H), 7.91 (d, J=4.4 Hz, 1H), 7.76-7.70 (m, 2H), 7.29 (d, J=8.4 Hz, 1H), 7.23 (s, 1H), 4.20-4.17 (m, 2H), 3.99 (t, J=8.8 Hz, 4H), 3.85 (t, J=9.2 Hz, 2H), 3.31-3.29 (m, 1H), 2.25 (s, 3H). LCMS (M+H⁺) m/z: 521.1.

### Example 117: Preparation of N-(4-methyl-3-(2-(methylsulfonamido)-8,9-dihydroimidazo[1′,2′:1,6]pyrido[2,3-d]pyrimidin-6-yl)phenyl)-4-(trifluoromethyl)picolinamide (Compound 117)

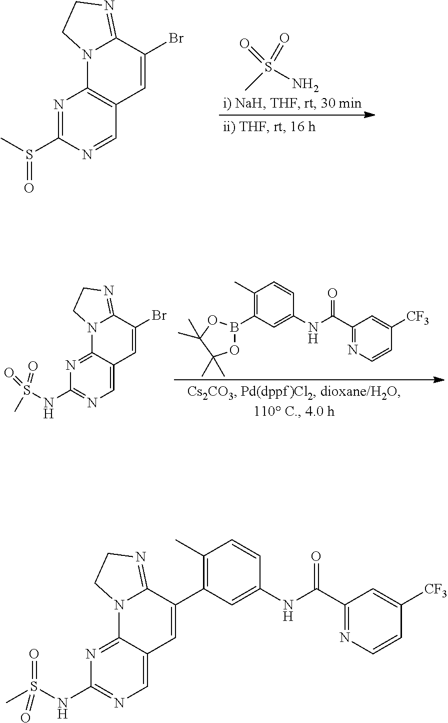

### Step 1: Synthesis of N-(6-bromo-8,9-dihydroimidazo[1′,2′:1,6]pyrido[2,3-d]pyrimidin-2-yl)methane sulfonamide

To a solution of methanesulfonamide (95 mg, 1.01 mmol) in dry-THF (4.0 mL), then was added NaH (40 mg, 1.01 mmol). The resulting mixture was stirred at r.t. for 30 min. then 6-bromo-2-(methylsulfinyl)-8,9-dihydroimidazo[1′,2′:1,6]pyrido[2,3-d]pyrimidine (158 mg, 0.505 mmol) was added, the reaction mixture was stirred at r.t. for 16 h. The reaction mixture was diluted with water (50 mL) and extracted with DCM (50 mL×3). The combined organic phase was washed with brine (50 mL), dried with Na₂SO₄, filtered and concentrated. The residue was purified by column chromatography (DCM/MeOH=10/1) to afford N-(6-bromo-8,9-dihydroimidazo[1′,2′:1,6]pyrido[2,3-d]pyrimidin-2-yl)methane sulfonamide (110 mg, 63.4% yield) as a pale yellow solid. LCMS (M+H⁺) m/z: 345.9 and 343.9.

### Step 2: Synthesis of N-(4-methyl-3-(2-(methylsulfonamido)-8,9-dihydroimidazo[1′,2′:1,6]pyrido[2,3-d]pyrimidin-6-yl)phenyl)-4-(trifluoromethyl)picolinamide

A mixture of N-(6-bromo-8,9-dihydroimidazo[1′,2′:1,6]pyrido[2,3-d]pyrimidin-2-yl)methane sulfonamide (30 mg, 0.087 mmol), N-(4-methyl-3-(4,4,5,5-tetramethyl-1,3,2-dioxaborolan-2-yl)phenyl)-4-(trifluoromethyl)picolinamide (39 mg, 0.096 mmol), Cs₂CO₃ (85 mg, 0.261 mmol) and Pd(dppf)Cl₂ (6.4 mg, 0.009 mmol) in dioxane (1.5 mL) and water (0.2 mL) was degassed and charged with N₂ three times and stirred at 100° C. for 16 h. The reaction mixture was concentrated and purified by Prep-HPLC (0.1% FA) to afford N-(4-methyl-3-(2-(methylsulfonamido)-8,9-dihydroimidazo[1′,2′:1,6]pyrido[2,3-d]pyrimidin-6-yl)phenyl)-4-(trifluoromethyl)picolinamide (6.2 mg, 13% yield) as a yellow solid. 1H NMR (400 MHz, DMSO-d₆): δ 10.81 (s, 1H), 9.03 (d, J=4.8 Hz, 1H), 8.47 (s, 1H), 8.34 (s, 1H), 8.09 (d, J=5.6 Hz, 1H), 7.87 (d, J=2.0 Hz, 1H), 7.82 (d, J=2.0 Hz, 1H), 7.45 (s, 1H), 7.29 (d, J=8.4 Hz, 1H), 4.21 (d, J=5.6 Hz, 2H), 3.97 (t, J=9.6 Hz, 2H), 3.19 (s, 3H), 2.21 (s, 3H). LCMS (M+H⁺) m/z: 544.4.

### Example 118: Preparation of N-(3-(2-acetamido-8,9-dihydroimidazo[1′,2′:1,6]pyrido[2,3-d]pyrimidin-6-yl)-4-methylphenyl)-4-(trifluoromethyl)picolinamide (Compound 118)

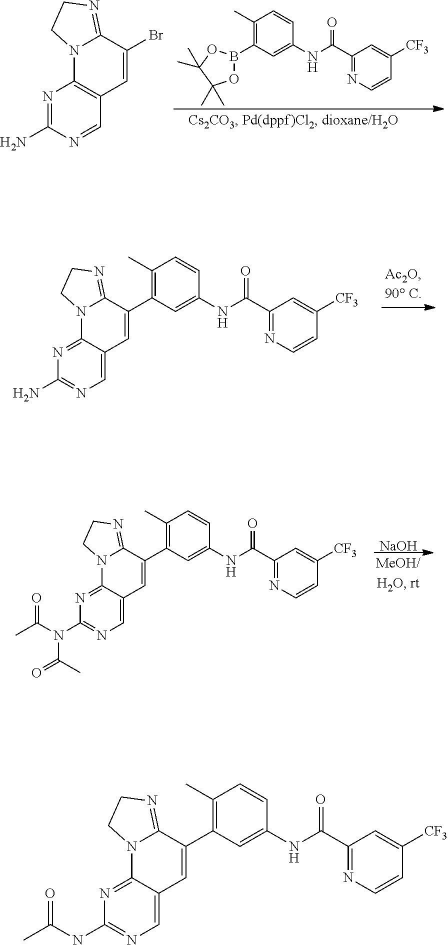

The preparation of N-(4-methyl-3-(4,4,5,5-tetramethyl-1,3,2-dioxaborolan-2-yl)phenyl)-4-(trifluoromethyl)picolinamide and 6-bromo-8,9-dihydroimidazo[1′,2′:1,6]pyrido[2,3-d]pyrimidin-2-amine was described in Example 76.

### Step 1: Synthesis of N-(3-(2-amino-8,9-dihydroimidazo[1′,2′:1,6]pyrido[2,3-d]pyrimidin-6-yl)-4-methylphenyl)-4-(trifluoromethyl)picolinamide

A mixture of 6-bromo-8,9-dihydroimidazo[1′,2′:1,6]pyrido[2,3-d]pyrimidin-2-amine (200 mg, 0.75 mmol), N-(4-methyl-3-(4,4,5,5-tetramethyl-1,3,2-dioxaborolan-2-yl)phenyl)-4-(trifluoromethyl) picolinamide (366 mg, 0.90 mmol), Cs₂CO₃ (735 mg, 2.56 mmol) and Pd(dppf)Cl₂ (30 mg, 0.05 mmol) in dioxane (10 mL) and water (1 mL) was degassed and charged with N₂ three times and stirred at 100° C. for 16 h. The reaction mixture was concentrated and the residue was purified by column chromatography (DCM/MeOH=10:1) to afford N-(3-(2-amino-8,9-dihydroimidazo[1′,2′:1,6]pyrido[2,3-d]pyrimidin-6-yl)-4-methylphenyl)-4-(trifluoromethyl)picolinamide (200 mg, 57% yield) as a brown solid. LCMS (M+H⁺) m/z: 466.2.

### Step 2: Synthesis of N-(3-(2-(N-acetylacetamido)-8,9-dihydroimidazo[1′,2′:1,6]pyrido[2,3-d]pyrimidin-6-yl)-4-methylphenyl)-4-(trifluoromethyl)picolinamide

A mixture of N-(3-(2-(N-acetylacetamido)-8,9-dihydroimidazo[1′,2′:1,6]pyrido[2,3-d]pyrimidin-6-yl)-4-methylphenyl)-4-(trifluoromethyl)picolinamide (130 mg, 0.32 mmol) in Ac₂O (10.0 mL) was stirred at 90° C. for 3 h. The mixture was concentrated in vacuum. The residue was purified by column chromatography (DCM/MeOH=10:1) to afford N-(3-(2-(N-acetylacetamido)-8,9-dihydroimidazo[1′,2′:1,6]pyrido[2,3-d]pyrimidin-6-yl)-4-methylphenyl)-4-(trifluoromethyl)picolinamide (130 mg, 73% yield) as a brown solid. LCMS (M+H⁺) m/z: 550.3.

### Step 3: Synthesis of N-(3-(2-acetamido-8,9-dihydroimidazo[1′,2′:1,6]pyrido[2,3-d]pyrimidin-6-yl)-4-methylphenyl)-4-(trifluoromethyl)picolinamide

A mixture of N-(3-(2-(N-acetylacetamido)-8,9-dihydroimidazo[1′,2′:1,6]pyrido[2,3-d]pyrimidin-6-yl)-4-methylphenyl)-4-(trifluoromethyl)picolinamide (130 mg, 0.24 mmol) in MeOH (5.0 mL) and H₂O (0.5 mL) was added NaOH (10 mg, 0.26 mmol). The mixture was stirred at rt for 16 h, concentrated and the residue was purified by Prep-TLC to give a yellow solid (40 mg), which was further purified by trituration with MeOH (2.0 mL) to afford N-(3-(2-acetamido-8,9-dihydroimidazo[1′,2′:1,6]pyrido[2,3-d]pyrimidin-6-yl)-4-methylphenyl)-4-(trifluoromethyl)picolinamide (11.7 mg, 9.7% yield) as a yellow solid. ¹H NMR (400 MHz, DMSO-d₆): δ 10.78 (s, 1H), 10.47 (s, 1H), 9.03 (d, J=5.2 Hz, 1H), 8.45 (s, 1H), 8.33 (s, 1H), 8.09 (d, J=4.8 Hz, 1H), 7.86 (d, J=2.4 Hz, 1H), 7.79 (dd, J=6.0, 2.0 Hz, 1H), 7.28-7.24 (m, 2H), 4.09 (t, J=9.2 Hz, 2H), 3.96 (t, J=8.8 Hz, 2H), 2.29 (s, 3H), 2.22 (s, 3H). LCMS (M+H⁺) m/z: 508.3.

### Example 119: Preparation of N-(3-(2-(cyclopropanecarboxamido)-8,9-dihydroimidazo[1′,2′:1,6]pyrido[2,3-d]pyrimidin-6-yl)-4-methylphenyl)-4-(trifluoromethyl)picolinamide (Compound 119)

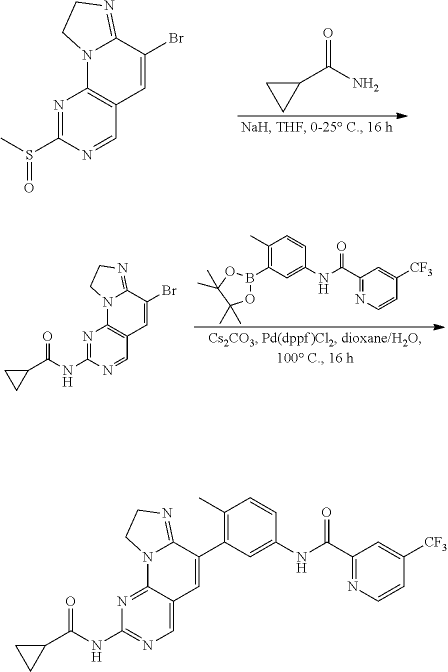

The preparation of N-(4-methyl-3-(4,4,5,5-tetramethyl-1,3,2-dioxaborolan-2-yl)phenyl)-4-(trifluoromethyl)picolinamide was described in Example 76.

The preparation of 6-bromo-2-(methylthio)-8,9-dihydroimidazo[1′,2′:1,6]pyrido[2,3-d]pyrimidine was described in Example 26.

### Step 1: Synthesis of N-(6-bromo-8,9-dihydroimidazo[1′,2′:1,6]pyrido[2,3-d]pyrimidin-2-yl)cyclopropanecarboxamide

To a solution of cyclopropanecarboxamide (81 mg 0.96 mmol) in dry THF (5 mL) was added NaH (81 mg 0.96 mmol) at 0° C. and the reaction mixture was stirred for 1 h, then 6-bromo-2-(methylsulfinyl)-8,9-dihydroimidazo[1′,2′:1,6]pyrido[2,3-d]pyrimidine (150 mg, 0.48 mmol) was added. The mixture was stirred at r.t. for 16 h. The reaction mixture was concentrated in vacuum and the residue was purified by column chromatography on silica gel (DCM/MeOH=10/1) to afford N-(6-bromo-8,9-dihydroimidazo[1′,2′:1,6]pyrido[2,3-d]pyrimidin-2-yl)cyclopropanecarboxamide (40 mg, 25% yield) as brown solid. LCMS (M+H⁺) m/z: 334.0 and 336.0.

### Step 2: Synthesis of N-(3-(2-(cyclopropanecarboxamido)-8,9-dihydroimidazo[1′,2′:1,6]pyrido[2,3-d]pyrimidin-6-yl)-4-methylphenyl)-4-(trifluoromethyl)picolinamide formic acid

A mixture of N-(6-bromo-8,9-dihydroimidazo[1′,2′:1,6]pyrido[2,3-d]pyrimidin-2-yl)cyclopropanecarboxamide (40 mg, 0.12 mmol), N-(4-methyl-3-(4,4,5,5-tetramethyl-1,3,2-dioxaborolan-2-yl)phenyl)-4-(trifluoromethyl)picolinamide (54 mg, 0.13 mmol), Cs₂CO₃ (116 mg, 0.36 mmol) and Pd(dppf)Cl₂ (4 mg, 0.005 mmol) in dioxane (5 mL) and water (0.5 mL) was degassed and charged with N₂ three times and stirred at 100° C. for 16 h. The reaction mixture was concentrated in vacuum and purified by column chromatography on silica gel (DCM/MeOH=10/1) to give crude product, which was purified by Prep-HPLC (0.1% FA) to afford N-(3-(2-(cyclopropanecarboxamido)-8,9-dihydroimidazo[1′,2′:1,6]pyrido[2,3-d]pyrimidin-6-yl)-4-methylphenyl)-4-(trifluoromethyl)picolinamide formic acid (15.9 mg, 23% yield) as a yellow solid. ¹H NMR (400 MHz, DMSO-d₆): δ 10.78 (s, 2H), 9.03 (d, J=5.2 Hz, 1H), 8.45 (s, 1H), 8.33 (s, 1H), 8.15 (s, 1H), 8.09 (d, J=4.4 Hz, 1H), 7.86 (d, J=2.0 Hz, 1H), 7.80 (d, J=8.4, 2.4 Hz, 1H), 7.29 (s, 1H), 7.25 (d, J=8.4 Hz, 1H), 4.08 (t, J=9.2 Hz, 2H), 3.96 (t, J=9.2 Hz, 2H), 2.54-2.50 (m, 1H), 2.22 (s, 3H), 0.84-0.82 (m, 4H). LCMS (M+H⁺) m/z: 534.8.

### Example 120: Preparation of N-(4-methyl-3-(5-methyl-2-(methylamino)-8,9-dihydroimidazo[1′,2′:1,6]pyrido[2,3-d]pyrimidin-6-yl)phenyl)-4-(trifluoromethyl)picolinamide (Compound 120)

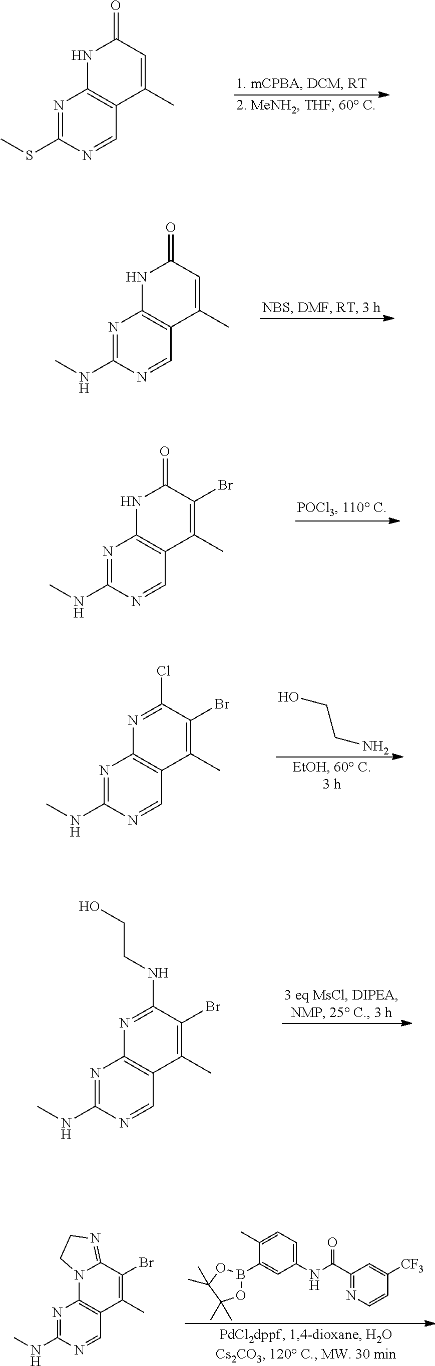

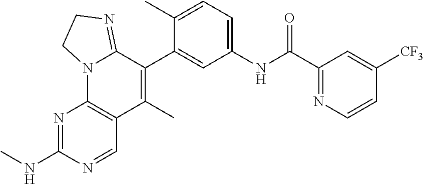 [SMILES: CNc1ncc2c(n1)N1CCN=C1C(c1cc(NC(=O)c3cc(C(F)(F)F)ccn3)ccc1C)=C2C]

### Step 1: Synthesis of 5-methyl-2-(methylamino)pyrido[2,3-d]pyrimidin-7(8H)-one

To a mixture of 5-methyl-2-(methylthio)pyrido[2,3-d]pyrimidin-7(8H)-one (100 mg, 0.5 mmol) in DCM (6 mL) was added m-CPBA (258 mg, 1.5 mmol) at rt and the reaction mixture was stirred for 2 h. The mixture was concentrated to give the crude product (300 mg) as white solid, which was used to next step without further purification. To a mixture of crude product (300 mg crude, 0.5 mmol) was added 2M MeNH₂ in THF (6 mL) at rt and the mixture was stirred for 3 h at 60° C. The mixture was monitored by LCMS. The reaction mixture was poured in to water, the precipitate was filtered and dried under vacuum to give 5-methyl-2-(methylamino)pyrido[2,3-d]pyrimidin-7(8H)-one (96 mg, 78.5% yield) as white solid. LCMS (M+H⁺) m/z 191.1.

### Step 2: Synthesis of 6-bromo-5-methyl-2-(methylamino)pyrido[2,3-d]pyrimidin-7(8H)-one

To a solution of 5-methyl-2-(methylamino)pyrido[2,3-d]pyrimidin-7(8H)-one (380 mg, 0.5 mmol) in DMF (10 mL) was added NBS (427 mg, 2.4 mmol) at rt and the reaction mixture was stirred at rt for 3 h. The reaction mixture was poured in to water, the precipitate was filtered and dried under vacuum to give 6-bromo-5-methyl-2-(methylamino)pyrido[2,3-d]pyrimidin-7(8H)-one (360 mg, 78.5% yield) as white solid. LCMS (M+H⁺) m/z 269.0.

### Step 3: Synthesis of 6-bromo-7-chloro-N,5-dimethylpyrido[2,3-d]pyrimidin-2-amine

A mixture of 6-bromo-5-methyl-2-(methylamino)pyrido[2,3-d]pyrimidin-7(8H)-one (380 mg, 0.37 mmol) in POCl₃ (5 mL) was stirred at 110° C. for 3 h. The reaction mixture was concentrated to give 6-bromo-7-chloro-N,5-dimethylpyrido[2,3-d]pyrimidin-2-amine (287 mg, 73% yield) as solid. LCMS (M+H⁺) m/z: 286.9.

### Step 4: Synthesis of 2-((6-bromo-5-methyl-2-(methylamino)pyrido[2,3-d]pyrimidin-7-yl)amino)ethan-1-ol

A mixture of 6-bromo-7-chloro-N,5-dimethylpyrido[2,3-d]pyrimidin-2-amine (287 mg, 1.0 mmol) and ethanolamine (5 mL) was stirred at 60° C. for 2 h. The reaction mixture was poured into water, and extracted with DCM (20 mL×2). The combined organic phase was concentrated to give 2-((6-bromo-5-methyl-2-(methylamino)pyrido[2,3-d]pyrimidin-7-yl)amino)ethan-1-ol (230 mg, 73% yield) as solid. LCMS (M+H⁺) m/z: 312.0.

### Step 5: Synthesis of 6-bromo-N,5-dimethyl-8,9-dihydroimidazo[1′,2′:1,6]pyrido[2,3-d]pyrimidin-2-amine

To a mixture of 2-((6-bromo-5-methyl-2-(methylamino)pyrido[2,3-d]pyrimidin-7-yl)amino)ethan-1-ol (170 mg, 0.5 mmol), DIPEA (645 mg, 5.0 mmol) in NMP (15 mL) was added and MsCl (310 mg, 2.5 mmol) at rt. The reaction mixture was stirred at 25° C. for 2 h. Water (30 mL) was added, the reaction mixture was extracted by EA (10 mL*3). The combined organic phase was washed with brine (20 mL) and purified by HPLC to give 6-bromo-N,5-dimethyl-8,9-dihydroimidazo[1′,2′:1,6]pyrido[2,3-d]pyrimidin-2-amine (130 mg) as solid. LCMS (M+H⁺) m/z: 293.9.

### Step 6: Synthesis of N-(4-methyl-3-(5-methyl-2-(methylamino)-8,9-dihydroimidazo[1′,2′:1,6]pyrido[2,3-d]pyrimidin-6-yl)phenyl)-4-(trifluoromethyl)picolinamide

The mixture of 6-bromo-N,5-dimethyl-8,9-dihydroimidazo[1′,2′:1,6]pyrido[2,3-d]pyrimidin-2-amine (30 mg, 0.1 mmol, 1.0 equiv), N-(4-methyl-3-(4,4,5,5-tetramethyl-1,3,2-dioxaborolan-2-yl)phenyl)-4-(trifluoromethyl)picolinamide (42 mg, 0.1 mmol, 1.0 equiv), K₂CO₃ (41 mg, 0.3 mmol, 3.0 equiv), and PdCl₂ (dppf) (14 mg, 0.02 mmol, 20 mol %) were suspended with 1,4-dioxane (2 mL) and H₂O (0.5 mL) was stirred at 110° C. for 3 h. The reaction mixture was purified by HPLC (0.5% FA) to give N-(4-methyl-3-(5-methyl-2-(methylamino)-8,9-dihydroimidazo[1′,2′:1,6]pyrido[2,3-d]pyrimidin-6-yl)phenyl)-4-(trifluoromethyl)picolinamide formate (5.6 mg) as yellow solid. ¹H NMR (400 MHz, CD₃OD): δ 10.76 (s, 1H), 9.02 (d, J=4.2 Hz, 1H), 8.47 (s, 1H), 8.32 (s, 1H), 8.16 (s, 1H), 8.07 (d, J=4.8 Hz, 1H), 7.79 (d, J=8.4 Hz, 1H), 7.71 (s, 1H), 7.70-7.60 (m, 1H), 7.30 (d, J=8.0 Hz, 1H), 3.87-3.82 (m, 2H), 3.63-3.52 (m, 2H), 3.06 (s, 3H), 2.07 (s, 3H), 1.98 (s, 3H). LCMS (M+H⁺) m/z: 494.0.

### Example 121: Preparation of N-(4-fluoro-3-(5-methyl-2-(methylamino)-8,9-dihydroimidazo[1′,2′:1,6]pyrido[2,3-d]pyrimidin-6-yl)phenyl)-4-(trifluoromethyl)picolinamide (Compound 121)

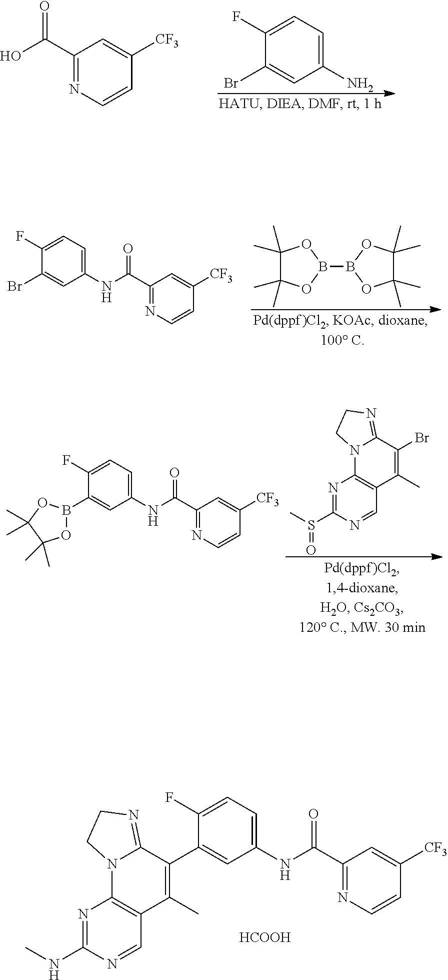

### Step 1: Synthesis of N-(3-bromo-4-fluorophenyl)-4-(trifluoromethyl)picolinamide

To a solution of 4-(trifluoromethyl)picolinic acid (500 mg, 2.6 mmol) in DMF (5.0 mL) was added HATU (1.48 g, 3.9 mmol), DIEA (1.00 g, 7.8 mmol), 3-bromo-4-fluoroaniline (551 mg, 2.9 mmol). The resulting mixture was stirred at r.t for 1 h. The reaction mixture was added H₂O (20 mL), extracted with EA (30 mL×3). The combined organic phases were washed with brine (30 mL), dried over Na₂SO₄, filtered and concentrated under vacuum. The resulting residue was purified by column chromatography (PE/EA=3/1) to afford N-(3-bromo-4-fluorophenyl)-4-(trifluoromethyl)picolinamide (809 mg, 86% yield) as a white solid. LCMS (M+H⁺) m/z: 363.0 and 365.0.

### Step 2: Synthesis of N-(4-fluoro-3-(4,4,5,5-tetramethyl-1,3,2-dioxaborolan-2-yl)phenyl)-4-(trifluoromethyl)picolinamide

A mixture of N-(3-bromo-4-fluorophenyl)-4-(trifluoromethyl)picolinamide (809 mg, 2.2 mmol) in dioxane (25 mL) was added 4,4,4′,4′,5,5,5′,5′-octamethyl-2,2′-bi(1,3,2-dioxaborolane) (1.68 g, 6.6 mmol), KOAc (650 mg, 6.6 mmol), Pd(dppf)Cl₂ (160 mg, 0.22 mmol). The mixture was degassed and charged with Ar for 3 times, stirred at 100° C. for 3 h. The reaction mixture was concentrated under vacuum and H₂O (50.0 mL) was added. The reaction mixture was extracted with EA (100 mL×3). The combined organic phases were washed with brine (100.0 mL), dried over Na₂SO₄, filtered and concentrated. The residue was purified by column chromatography (PE/EA=10/1) to afford N-(4-fluoro-3-(4,4,5,5-tetramethyl-1,3,2-dioxaborolan-2-yl)phenyl)-4-(trifluoromethyl)picolinamide (267 mg, 90% purity) as a brown solid. LCMS (M+H⁺) m/z: 411.0.

### Step 3: Synthesis of N-(4-fluoro-3-(5-methyl-2-(methylamino)-8,9-dihydroimidazo[1′,2′:1,6]pyrido[2,3-d]pyrimidin-6-yl)phenyl)-4-(trifluoromethyl)picolinamide

The mixture of 6-bromo-N,5-dimethyl-8,9-dihydroimidazo[1′,2′:1,6]pyrido[2,3-d]pyrimidin-2-amine (30 mg, 0.1 mmol, 1.0 equiv), N-(4-fluoro-3-(4,4,5,5-tetramethyl-1,3,2-dioxaborolan-2-yl)phenyl)-4-(trifluoromethyl)picolinamide (41 mg, 0.1 mmol, 1.0 equiv), Cs₂CO₃ (100 mg, 0.3 mmol, 3.0 equiv), and PdCl₂(dppf) (15 mg, 0.02 mmol, 20 mol %) in 1,4-dioxane (3 mL) and H₂O (0.2 mL was stirred at 120° C. for 40 min under MW. The reaction mixture was purified by flash (DCM:MeOH=10:1) to give 15 mg crude product, which was further purified by HPLC to give pure product N-(4-fluoro-3-(5-methyl-2-(methylamino)-8,9-dihydroimidazo[1′,2′:1,6]pyrido[2,3-d]pyrimidin-6-yl)phenyl)-4-(trifluoromethyl)picolinamide (8.9 mg) as yellow solid. ¹H NMR (400 MHz, CD₃OD): δ 8.96 (d, J=4.8 Hz, 1H), 8.87 (s, 1H), 8.52 (s, 1H), 8.42 (s, 1H), 7.96-7.92 (m, 3H), 7.36 (t, J=9.6 Hz, 1H), 4.65-4.62 (m, 2H), 4.07-4.03 (m, 2H), 3.05 (s, 3H), 2.36 (s, 3H). LCMS (M+H⁺) m/z: 498.0.

### Example 122: Preparation of N-(4-methyl-3-(4-methyl-2-(methylamino)-8,9-dihydroimidazo[1′,2′:1,6]pyrido[2,3-d]pyrimidin-6-yl)phenyl)-4-(trifluoromethyl)picolinamide (Compound 122)

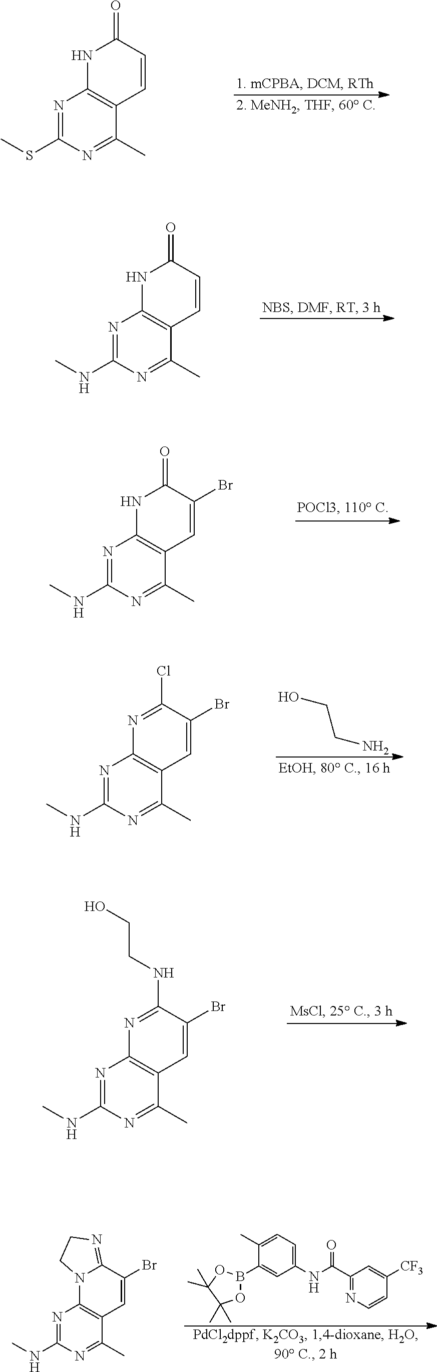

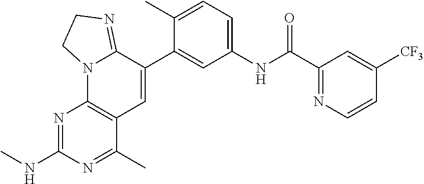 [SMILES: CNc1nc(C)c2c(n1)N1CCN=C1C(c1cc(NC(=O)c3cc(C(F)(F)F)ccn3)ccc1C)=C2]

### Step 1: Synthesis of 4-methyl-2-(methylamino)pyrido[2,3-d]pyrimidin-7(8H)-one

To a solution of 4-methyl-2-(methylthio)pyrido[2,3-d]pyrimidin-7(8H)-one (500 mg, 2.41 mmol) in DCM (10 mL) was added m-CPBA (1.04 g, 6 mmol), the reaction mixture was stirred at r.t. for 2 h. LCMS showed the reaction completed. The reaction mixture was concentrated to give a yellow solid. To the crude solid in THF (5 mL) was added 2M methylamine in THF (12 mL, 24 mmol). The mixture was stirred at 60° C. for 2 h. LCMS showed the reaction completed. The mixture was diluted to water (10 mL), extracted by EA (10 mL*3). The combined organic phase was washed with brine (20 mL), dried over anhydrous Na₂SO₄, filtered and concentrated to afford a crude solid, which was purified by prep-HPLC to afford 4-methyl-2-(methylamino)pyrido[2,3-d]pyrimidin-7(8H)-one (400 mg, 72% yield) as yellow solid. LCMS (M+H⁺) m/z: 191.1.

### Step 2: Synthesis of 6-bromo-4-methyl-2-(methylamino)pyrido[2,3-d]pyrimidin-7(8H)-one

To a solution of 4-methyl-2-(methylamino)pyrido[2,3-d]pyrimidin-7(8H)-one (400 mg, 2.1 mmol) in DMF (6 mL) was added NBS (409 mg, 2.3 mmol). The mixture was stirred at 25° C. for 2 h. LCMS showed the reaction completed. The reaction mixture was diluted to water (20 mL). The resulting red precipitated solid was filtered and the cake was washed by water (10 mL). The solid was dried to afford 6-bromo-4-methyl-2-(methylamino)pyrido[2,3-d]pyrimidin-7(8H)-one (450 mg, 80% yield) as a red solid. LCMS (M+H+) m/z: 269.0.

### Step 3: Synthesis of 6-bromo-7-chloro-N,4-dimethylpyrido[2,3-d]pyrimidin-2-amine

The mixture of 6-bromo-4-methyl-2-(methylamino)pyrido[2,3-d]pyrimidin-7(8H)-one (450 mg, 1.67 mmol) in POCl₃ (5 mL) was stirred for 2 h at 110° C. LCMS showed the reaction completed. The reaction mixture was concentrated to obtained a crude oil, which was diluted to saturated NaHCO₃a.q. (10 mL), extracted by EA (10 mL*3). The combined organic phase was washed by brine (20 mL), dried over anhydrous Na₂SO₄, filtered and concentrated to afford crude 6-bromo-7-chloro-N,4-dimethylpyrido[2,3-d]pyrimidin-2-amine (450 mg, crude), which was used for next step without further purification. LCMS (M+H+) m/z: 286.9.

### Step 4: Synthesis of 2-((6-bromo-4-methyl-2-(methylamino)pyrido[2,3-d]pyrimidin-7-yl)amino)ethan-1-ol

To a mixture of 6-bromo-7-chloro-N,4-dimethylpyrido[2,3-d]pyrimidin-2-amine (450 mg, 1.56 mmol) in EtOH (2 mL) was added 2-aminoethan-1-ol (571 mg, 9.36 mmol). The mixture was stirred for 16 h at 80° C. LCMS showed the reaction completed. The reaction mixture was concentrated and diluted to water (10 mL), extracted by DCM/MeOH=10:1 (10 mL*5). The combined organic phase was washed with brine (30 mL), dried over anhydrous Na₂SO₄, filtered and concentrated to give 2-((6-bromo-4-methyl-2-(methylamino)pyrido[2,3-d]pyrimidin-7-yl)amino)ethan-1-ol (310 mg, crude), which was used for next step without further purification. LCMS (M+H+) m/z: 311.9.

### Step 5: Synthesis of 6-bromo-N,4-dimethyl-8,9-dihydroimidazo[1′,2′:1,6]pyrido[2,3-d]pyrimidin-2-amine

The mixture of 2-((6-bromo-4-methyl-2-(methylamino)pyrido[2,3-d]pyrimidin-7-yl)amino)ethan-1-ol (310 mg, 1 mmol) in THF (5 mL) was added MsCl (229 mg, 2 mmol). The mixture was stirred for 3 h at 25° C. LCMS showed the reaction completed. The reaction mixture was diluted to water (5 mL), extracted by EA (10 mL*3). The combined organic phase was washed by brine (20 mL), dried over anhydrous Na₂SO₄, filtered and concentrated. The residue was purified by column chromatography to afford 6-bromo-N,4-dimethyl-8,9-dihydroimidazo[1′,2′:1,6]pyrido[2,3-d]pyrimidin-2-amine (210 mg, 72% yield). LCMS (M+H+) m/z: 294.0.

### Step 6: Synthesis of N-(4-methyl-3-(4-methyl-2-(methylamino)-8,9-dihydroimidazo[1′,2′:1,6]pyrido[2,3-d]pyrimidin-6-yl)phenyl)-4-(trifluoromethyl)picolinamide

To a solution of 6-bromo-N,4-dimethyl-8,9-dihydroimidazo[1′,2′:1,6]pyrido[2,3-d]pyrimidin-2-amine (40 mg, 0.14 mmol) in dioxane (3 mL) and water (0.6 mL) was added N-(4-methyl-3-(4,4,5,5-tetramethyl-1,3,2-dioxaborolan-2-yl)phenyl)-4-(trifluoromethyl)picolinamide (55 mg, 0.14 mmol), Pd(dppf)Cl₂ (10 mg, 0.014 mmol) and K₂CO₃ (39 mg, 0.28 mmol). The mixture was bubbled under nitrogen for 5 min and then stirred at 90° C. for 2 h. LCMS showed the reaction completed. The reaction mixture was filtered by celite, the filtration was diluted to water (5 mL), extracted by EA (5 mL*3). The combined organic phase was washed by brine (10 mL), dried over anhydrous Na₂SO₄ to give a crude oil. The crude oil was purified by Prep-HPLC to afford N-(4-methyl-3-(4-methyl-2-(methylamino)-8,9-dihydroimidazo[1′,2′:1,6]pyrido[2,3-d]pyrimidin-6-yl)phenyl)-4-(trifluoromethyl)picolinamide (8.4 mg) as yellow solid. ¹H NMR (400 MHz, DMSO-d₆): δ 10.75 (s, 1H), 9.03 (d, J=4.8 Hz, 1H), 8.34 (s, 1H), 8.22 (s, 1H), 8.09 (d, J=4.4 Hz, 1H), 7.81 (d, J=4.8 Hz, 2H), 7.47-7.36 (m, 2H), 7.24 (d, J=8.8 Hz, 1H), 4.12-4.00 (m, 2H), 3.90 (t, J=9.6 Hz, 2H), 2.85 (d, J=4.8 Hz, 3H), 2.37 (s, 3H), 2.21 (s, 3H). LCMS (M+H⁺) m/z: 494.0.

### Example 123: Preparation of N-(4-chloro-3-(4-methyl-2-(methylamino)-8,9-dihydroimidazo[1′,2′:1,6]pyrido[2,3-d]pyrimidin-6-yl)phenyl)-4-(trifluoromethyl)picolinamide (Compound 123)

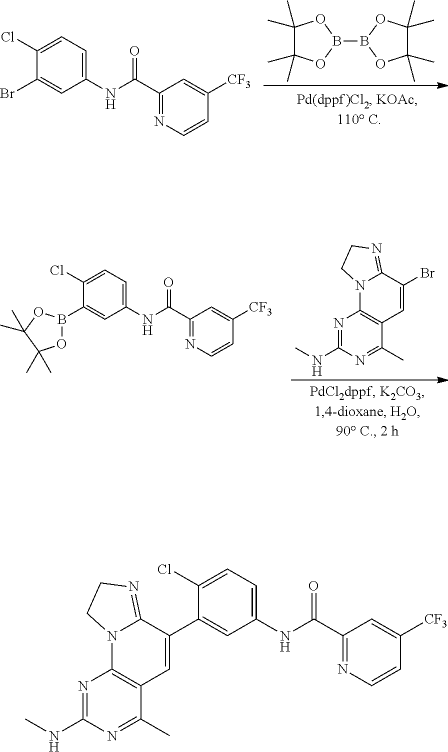

### Step 1: Synthesis of N-(4-chloro-3-(4,4,5,5-tetramethyl-1,3,2-dioxaborolan-2-yl)phenyl)-4-(trifluoromethyl) picolinamide

To a solution of N-(3-bromo-4-chlorophenyl)-4-(trifluoromethyl)picolinamide (1.9 g, 5.0 mmol) in dioxane (30 mL) was added 4,4,4′,4′,5,5,5′,5′-octamethyl-2,2′-bi(1,3,2-dioxaborolane) (2.54 g, 10.0 mmol), Pd(dppf)Cl₂ (365 mg, 0.50 mmol), KOAc (1.47 g, 15 mmol). The reaction mixture was stirred for 16 h at 110° C. The reaction mixture was filtered with celite. The filtrate was concentrated under vacuum. The residue was triturated with PE and filtered to afford N-(4-chloro-3-(4,4,5,5-tetramethyl-1,3,2-dioxaborolan-2-yl)phenyl)-4-(trifluoromethyl) picolinamide (1.2 g, crude) as a grey solid. LCMS (M+H⁺) m/z: 427.0.

### Step 2: Synthesis of N-(4-chloro-3-(4-methyl-2-(methylamino)-8,9-dihydroimidazo[1′,2′:1,6]pyrido[2,3-d]pyrimidin-6-yl)phenyl)-4-(trifluoromethyl)picolinamide

To a solution of 6-bromo-N,4-dimethyl-8,9-dihydroimidazo[1′,2′:1,6]pyrido[2,3-d]pyrimidin-2-amine (40 mg, 0.14 mmol) in dioxane (3 mL) and water (0.6 mL) was added N-(4-chloro-3-(4,4,5,5-tetramethyl-1,3,2-dioxaborolan-2-yl)phenyl)-4-(trifluoromethyl)picolinamide (58 mg, 0.14 mmol), Pd(dppf)Cl₂ (10 mg, 0.014 mmol) and K₂CO₃ (39 mg, 0.28 mmol). The mixture was bubbled by nitrogen for 5 min and then stirred at 90° C. for 2 h. LCMS showed the reaction completed. The reaction mixture was filtered by celite, the filtration was diluted to water (5 mL), extracted by EA (5 mL*3). The combined organic phase was washed by brine (10 mL), dried over anhydrous Na₂SO₄ to give a crude oil. The crude oil was purified by Prep-HPLC to afford N-(4-chloro-3-(4-methyl-2-(methylamino)-8,9-dihydroimidazo[1′,2′:1,6]pyrido[2,3-d]pyrimidin-6-yl)phenyl)-4-(trifluoromethyl)picolinamide (14 mg) as yellow solid. ¹H NMR (400 MHz, DMSO-d₆): δ 11.00 (s, 1H), 9.04 (d, J=5.2 Hz, 1H), 8.35 (s, 1H), 8.18 (s, 1H), 8.11 (d, J=4.0 Hz, 1H), 8.06 (s, 1H), 7.95 (dd, J=8.8 Hz, 2.4 Hz, 1H), 7.56-7.46 (m, 3H), 4.14-3.93 (m, 2H), 3.90 (t, J=9.6 Hz, 2H), 2.85 (d, J=4.8 Hz, 3H), 2.37 (s, 3H). LCMS (M+H⁺) m/z: 514.0.

### Example 124: Preparation of N-(4-methyl-3-(2-(methylamino)-8,9-dihydroimidazo[2,1-h]pteridin-6-yl)phenyl)-4-(trifluoromethyl)picolinamide (Compound 124)

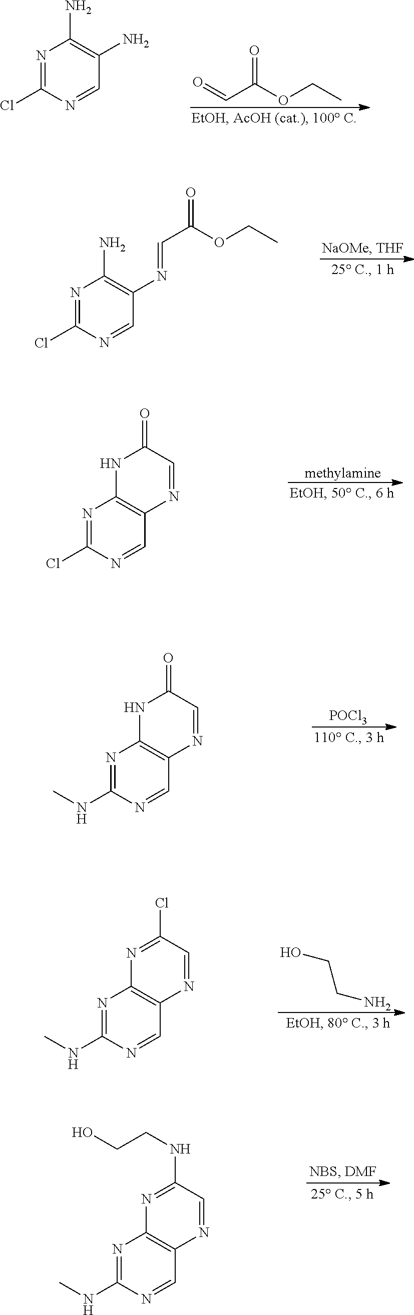

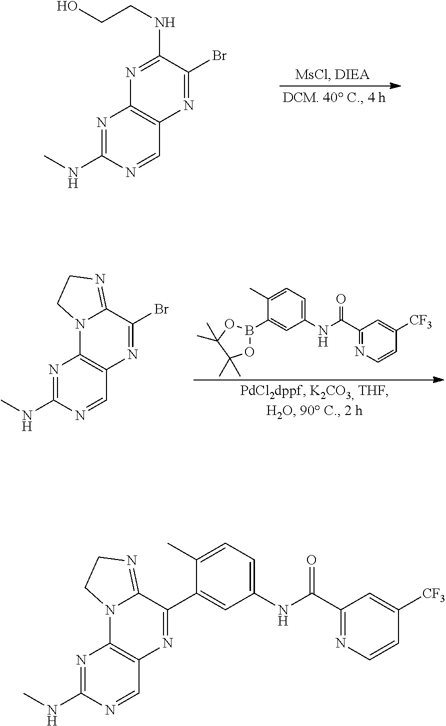

### Step 1: Synthesis of ethyl 2-((4-amino-2-chloropyrimidin-5-yl)imino)acetate

To a solution of 2-chloropyrimidine-4,5-diamine (2.88 g, 20 mmol) in EtOH (30 mL) was added ethyl 2-oxoacetate (50% w/w % in toluene) (4.08 g, 20 mmol) and AcOH (2 drops), the reaction mixture was stirred at 100° C. for 2 h. LCMS showed the reaction completed. The reaction mixture was filtered to obtain a yellow solid. The crude solid was dried under vacuum to give ethyl 2-((4-amino-2-chloropyrimidin-5-yl)imino)acetate (2.1 g) as yellow solid. LCMS (M+H⁺) m/z: 228.9.

### Step 2: Synthesis of 2-chloropteridin-7(8H)-one

To a solution of ethyl 2-((4-amino-2-chloropyrimidin-5-yl)imino)acetate (2.1 g, 9.2 mmol) in THF (20 mL) was added NaOMe (993 mg, 18.4 mmol). The mixture was stirred at 25° C. for 1 h. LCMS showed the reaction completed. The reaction mixture was diluted to water (20 mL), the resulting precipitate was filtered and the cake was washed by water (10 mL). The solid was dried to afford 2-chloropteridin-7(8H)-one (830 mg, 50% yield) as a red solid. LCMS (M+H+) m/z: 183.0.

### Step 3: Synthesis of 2-(methylamino)pteridin-7(8H)-one

To a mixture of 2-chloropteridin-7(8H)-one (830 mg, 4.6 mmol) in EtOH (5 mL) was added methylamine (33% in EtOH) (5 mL), the reaction mixture was stirred for 6 h at 50° C. LCMS showed the reaction completed. The reaction mixture was concentrated to give 2-(methylamino)pteridin-7(8H)-one (650 mg, crude), which was used for next step without further purification. LCMS (M+H⁺) m/z: 178.0.

### Step 4: Synthesis of 7-chloro-N-methylpteridin-2-amine

The mixture of 2-(methylamino)pteridin-7(8H)-one (400 mg, 2.26 mmol) in POCl₃ (5 mL) was stirred for 3 h at 110° C. LCMS showed the reaction completed. The reaction mixture was concentrated to give a crude oil, which was diluted to saturated NaHCO₃a.q. (10 mL), extracted by EA (10 mL*3). The combined organic phase was washed with brine (20 mL), dried over anhydrous Na₂SO₄, filtered and concentrated to afford 7-chloro-N-methylpteridin-2-amine (600 mg, crude). The crude solid was used for next step without further purification. LCMS (M+H⁺) m/z: 196.1.

### Step 5: Synthesis of 2-((2-(methylamino)pteridin-7-yl)amino)ethan-1-ol

The mixture of 7-chloro-N-methylpteridin-2-amine (600 mg, crude) in EtOH (6 mL) was added 2-aminoethan-1-ol (1.22 g, 20 mmol). The mixture was stirred for 3 h at 80° C. LCMS showed the reaction completed. The reaction mixture was concentrated and diluted to water (10 mL), extracted with DCM/MeOH=10:1 (10 mL*5). The combined organic phase was washed by brine (30 mL), dried over anhydrous Na₂SO₄, filtered and concentrated to give crude product, which was purified by prep-HPLC to give 2-((2-(methylamino)pteridin-7-yl)amino)ethan-1-ol (300 mg) as white solid. LCMS (M+H+) m/z: 221.1.

### Step 6: Synthesis of 2-((6-bromo-2-(methylamino)pteridin-7-yl)amino)ethan-1-ol

To a mixture of 2-((2-(methylamino)pteridin-7-yl)amino)ethan-1-ol (220 mg, 1 mmol) in DMF (5 mL) was added NBS (356 mg, 2 mmol). The mixture was stirred for 5 h at 25° C. LCMS showed the reaction completed. The reaction mixture was diluted to water (5 mL), extracted by EA (10 mL*3). The combined organic phase was washed with brine (20 mL), dried over anhydrous Na₂SO₄, filtered and concentrated. The residue was purified by column chromatography to afford 2-((6-bromo-2-(methylamino)pteridin-7-yl)amino)ethan-1-ol (150 mg, 50% yield). LCMS (M+H+) m/z: 299.0.

### Step 7: Synthesis of 6-bromo-N-methyl-8,9-dihydroimidazo[2,1-h]pteridin-2-amine

To a mixture of 2-((6-bromo-2-(methylamino)pteridin-7-yl)amino)ethan-1-ol (150 mg, 0.5 mmol) in DCM (3 mL) was added DIEA (129 mg, 1 mmol) and MsCl (115 mg, 1 mmol). The mixture was stirred for 4 h at 25° C. LCMS showed the reaction completed. The reaction mixture was diluted to water (5 mL), extracted by EA (10 mL*3). The combined organic phase was washed with brine (20 mL), dried over anhydrous Na₂SO₄, filtered and concentrated. The residue was purified by column chromatography to afford 6-bromo-N-methyl-8,9-dihydroimidazo[2,1-h]pteridin-2-amine (80 mg, 57% yield). LCMS (M+H+) m/z: 281.0.

### Step 8: Synthesis of N-(4-methyl-3-(2-(methylamino)-8,9-dihydroimidazo[2,1-h]pteridin-6-yl)phenyl)-4-(trifluoromethyl)picolinamide

To a solution of 6-bromo-N-methyl-8,9-dihydroimidazo[2,1-h]pteridin-2-amine (40 mg, 0.14 mmol) in THF (3 mL) and water (0.6 mL) was added N-(4-methyl-3-(4,4,5,5-tetramethyl-1,3,2-dioxaborolan-2-yl)phenyl)-4-(trifluoromethyl)picolinamide (57 mg, 0.14 mmol), Pd(dppf)Cl₂ (10 mg, 0.014 mmol) and K₂CO₃ (39 mg, 0.28 mmol). The mixture was bubbled by nitrogen for 5 min and then stirred at 90° C. for 2 h. LCMS showed the reaction completed. The reaction mixture was filtered by celite, the filtration was diluted to water (5 mL), extracted by EA (5 mL*3). The combined organic phase was washed with brine (10 mL), dried over anhydrous Na₂SO₄ to give a crude oil. The crude oil was purified by Prep-HPLC to afford N-(4-methyl-3-(2-(methylamino)-8,9-dihydroimidazo[2,1-h]pteridin-6-yl)phenyl)-4-(trifluoromethyl)picolinamide (3.1 mg) as yellow solid. ¹H NMR (400 MHz, CD3OD): δ 8.86 (d, J=5.0 Hz, 1H), 8.33 (s, 1H), 8.23 (s, 1H), 7.83 (dd, J=9.6, 3.2 Hz, 2H), 7.74 (dd, J=8.4, 2.4 Hz, 1H), 7.25 (d, J=8.4 Hz, 1H), 4.16 (br s, 2H), 4.01 (t, J=9.6 Hz, 2H), 2.90 (s, 3H), 2.24 (s, 3H). LCMS (M+H⁺) m/z: 481.1.

### Example 125: Preparation of N-(4-methyl-3-(8-methyl-2-(methylamino)-8,9-dihydroimidazo[1′,2′:1,6]pyrido[2,3-d]pyrimidin-6-yl)phenyl)-4-(trifluoromethyl)picolinamide (Compound 125)

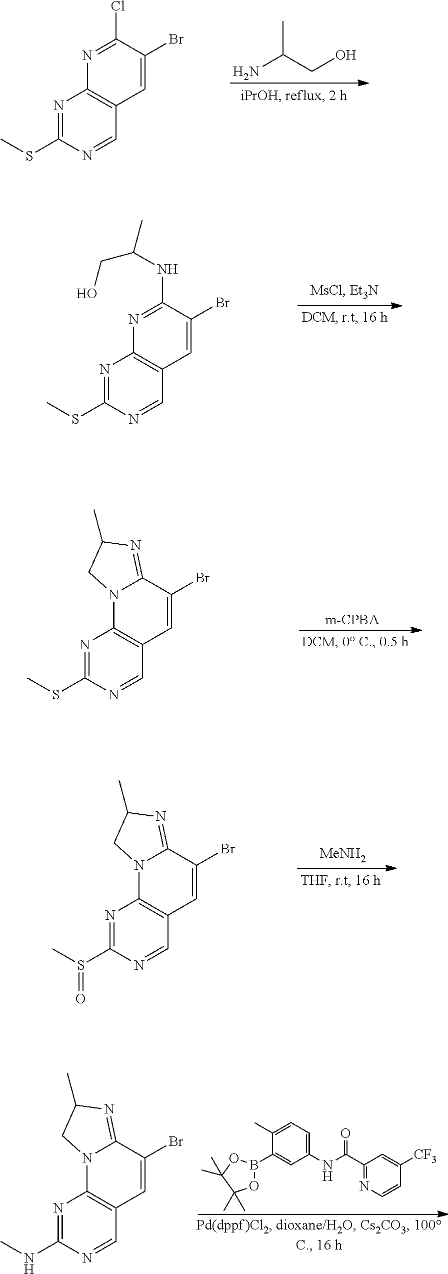

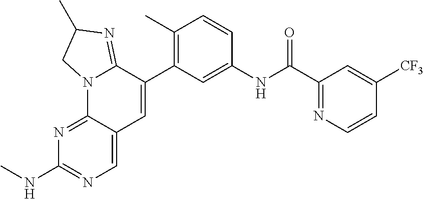 [SMILES: CNc1ncc2c(n1)N1CC(C)N=C1C(c1cc(NC(=O)c3cc(C(F)(F)F)ccn3)ccc1C)=C2]

### Step 1: Synthesis of 2-((6-bromo-2-(methylthio)pyrido[2,3-d]pyrimidin-7-yl)amino)propan-1-ol

A mixture of 6-bromo-7-chloro-2-(methylthio)pyrido[2,3-d]pyrimidine (1.0 g, 3.45 mmol), 2-aminopropan-1-ol (517 mg, 6.89 mmol) in iPrOH (15.0 mL) was stirred under reflux for 3.0 h. The reaction mixture was concentrated. Water was added, the precipitate was filtered to afford the 2-((6-bromo-2-(methylthio)pyrido[2,3-d]pyrimidin-7-yl)amino)propan-1-ol (1.11 g, 98% yield) as an off-white solid. LCMS (M+H+) m/z: 329.0 and 331.0.

### Step 2: Synthesis of 6-bromo-8-methyl-2-(methylthio)-8,9-dihydroimidazo[1′,2′:1,6]pyrido[2,3-d]pyrimidine

To a mixture of 2-((6-bromo-2-(methylthio)pyrido[2,3-d]pyrimidin-7-yl)amino)propan-1-ol (1.16 g, 3.5 mmol), TEA (1.06 g, 10.5 mmol) in DCM (20.0 mL), was added MsCl (1.0 g, 8.8 mmol). The resulting mixture was stirred at rt for 16 h. The reaction mixture was concentrated, diluted with cooled water (50 mL) and extracted with EA (50 mL×3). The combined organic phase was washed with brine (100 mL), dried with Na₂SO₄, filtered and concentrated. The residue was purified by column chromatography (PE/EA=3/1) to afford 6-bromo-8-methyl-2-(methylthio)-8,9-dihydroimidazo[1′,2′:1,6]pyrido[2,3-d]pyrimidine (1.0 g, 91% yield) as a yellow solid. LCMS (M+H⁺) m/z: 311.0 and 313.0.

### Step 3: Synthesis of 6-bromo-8-methyl-2-(methylsulfinyl)-8,9-dihydroimidazo[1′,2′:1,6]pyrido[2,3-d]pyrimidine

To a solution of 6-bromo-8-methyl-2-(methylthio)-8,9-dihydroimidazo[1′,2′:1,6]pyrido[2,3-d]pyrimidine (250 mg, 0.8 mmol) in dry DCM (20 mL) was added m-CPBA (368 mg, 1.6 mmol) at 0° C. The resulting mixture was stirred at 0° C. for 30 min. The reaction mixture was concentrated at r.t. under vacuum to afford crude 6-bromo-8-methyl-2-(methylsulfinyl)-8,9-dihydroimidazo[1′,2′:1,6]pyrido[2,3-d]pyrimidine (250 mg, crude) as a yellow solid, which was used in the next step without purification. LCMS (M+H)⁺ m/z: 326.9 and 328.9.

### Step 4: Synthesis of 6-bromo-N,8-dimethyl-8,9-dihydroimidazo[1′,2′:1,6]pyrido[2,3-d]pyrimidin-2-amine

To a solution of crude 6-bromo-8-methyl-2-(methylsulfinyl)-8,9-dihydroimidazo[1′,2′:1,6]pyrido[2,3-d]pyrimidine (250 mg, 0.76 mmol) in THF (20.0 mL) was added MeNH₂ (2 M in THF, 1.5 mL, 3.0 mmol). The mixture was stirred at r.t. for 16 h and concentrated. Water (80 mL) was added, and the reaction mixture was extracted with EA (50 mL×3). The combined organic phase was washed with brine (100 mL), dried with Na₂SO₄, filtered and concentrated. The residue was added into EA (4.0 mL), stirred at r.t. for 3.0 h, filtered to afford 6-bromo-N,8-dimethyl-8,9-dihydroimidazo[1′,2′:1,6]pyrido[2,3-d]pyrimidin-2-amine (200 mg, 88% yield) as a yellow solid. LCMS (M+H⁺) m/z: 294.0 and 296.0.

### Step 5: Synthesis of N-(4-methyl-3-(8-methyl-2-(methylamino)-8,9-dihydroimidazo[1′,2′:1,6]pyrido[2,3-d]pyrimidin-6-yl)phenyl)-4-(trifluoromethyl)picolinamide

A mixture of 6-bromo-N,8-dimethyl-8,9-dihydroimidazo[1′,2′:1,6]pyrido[2,3-d]pyrimidin-2-amine (200 mg, 0.68 mmol), N-(4-methyl-3-(4,4,5,5-tetramethyl-1,3,2-dioxaborolan-2-yl)phenyl)-4-(trifluoromethyl)picolinamide (276 mg, 0.68 mmol), Cs₂CO₃ (665 mg, 2.04 mmol) and Pd(dppf)Cl₂ (75 mg, 0.102 mmol) in dioxane (15.0 mL) and water (3 mL) was degassed and changed with N₂ three times and stirred at 100° C. for 16 h. The reaction mixture was diluted with water (80 mL), extracted with EA (50 mL×3). The combined organic phase was washed with brine (100 mL), dried with Na₂SO₄, filtered and concentrated. The residue was purified by Prep-HPLC (0.1% HCOOH) to afford N-(4-methyl-3-(8-methyl-2-(methylamino)-8,9-dihydroimidazo[1′,2′:1,6]pyrido[2,3-d]pyrimidin-6-yl)phenyl)-4-(trifluoromethyl)picolinamide (119.4 mg, 35% yield) as a yellow solid. ¹H NMR (400 MHz, DMSO-d₆): δ 10.75 (s, 1H), 9.03 (d, J=5.2 Hz, 1H), 8.33 (s, 1H), 8.21 (s, 1H), 8.08-8.09 (m, 1H), 7.81 (d, J=2.0 Hz, 2H), 7.78 (d, J=2.0 Hz, 1H), 7.76 (d, J=2.4 Hz, 1H), 7.38-7.43 (m, 1H), 7.22 (d, J=8.4 Hz, 1H), 7.15 (s, 1H), 4.21-4.24 (m, 2H), 3.52-3.63 (m, 1H), 2.85 (d, J=4.4 Hz, 3H), 2.21 (s, 3H), 1.20 (d, J=6.4 Hz, 3H). LCMS (M+H⁺) m/z: 494.4.

### Example 126 and 127: Preparation of (S)—N-(4-methyl-3-(8-methyl-2-(methylamino)-8,9-dihydroimidazo[1′,2′:1,6]pyrido[2,3-d]pyrimidin-6-yl)phenyl)-4-(trifluoromethyl)picolinamide (Compound 126) and (R)—N-(4-methyl-3-(8-methyl-2-(methylamino)-8,9-dihydroimidazo[1′,2′:1,6]pyrido[2,3-d]pyrimidin-6-yl)phenyl)-4-(trifluoromethyl)picolinamide (Compound 127)

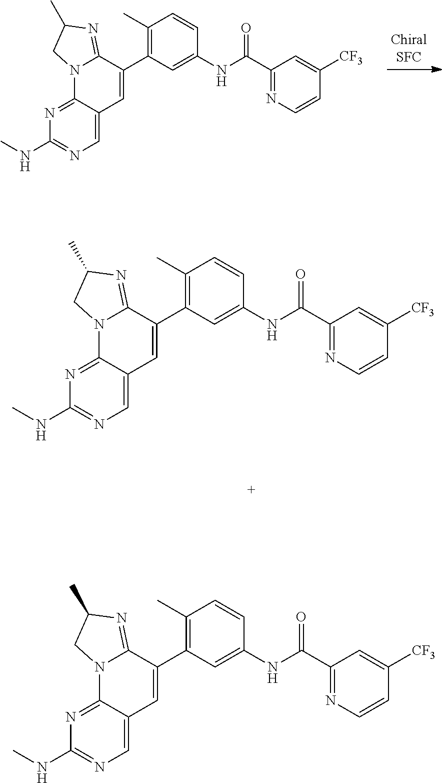 [SMILES: CNc1ncc2c(n1)N1CC(C)N=C1C(c1cc(NC(=O)c3cc(C(F)(F)F)ccn3)ccc1C)=C2 ; CNc1ncc2c(n1)N1C[C@H](C)N=C1C(c1cc(NC(=O)c3cc(C(F)(F)F)ccn3)ccc1C)=C2 ; CNc1ncc2c(n1)N1C[C@@H](C)N=C1C(c1cc(NC(=O)c3cc(C(F)(F)F)ccn3)ccc1C)=C2]

Compound 125 (60 mg) was separated by Chiral-HPLC to afford Compound 126 (12.9 mg) and Compound 127 (12.5 mg). Compound 126: ¹H NMR (400 MHz, DMSO-d₆): δ 10.76 (s, 1H), 9.03 (d, J=4.8 Hz, 1H), 8.33 (s, 1H), 8.28-8.22 (m, 1H), 8.08 (d, J=4.4 Hz, 1H), 7.82 (s, 1H), 7.80-7.77 (m, 1H), 7.53-7.41 (m, 1H), 7.25-7.21 (m, 1H), 4.25 (s, 2H), 3.66 (s, 1H), 2.86 (s, 3H), 2.21 (s, 3H), 1.22-1.21 (m, 3H). LCMS (M+H⁺) m/z: 494.4. Compound 127: ¹H NMR (400 MHz, DMSO-d₆): δ 10.76 (s, 1H), 9.03 (d, J=4.8 Hz, 1H), 8.33 (s, 1H), 8.26 (s, 1H), 8.21 (s, 1H), 8.09-8.08 (m, 1H), 7.82-7.81 (m, 1H), 7.79-7.77 (m, 1H), 7.49-7.41 (m, 1H), 7.23 (d, J=8.4 Hz, 1H), 7.19 (s, 1H), 4.24 (s, 2H), 3.63 (s, 1H), 2.86 (d, J=3.2 Hz, 3H), 2.21 (s, 3H), 1.22-1.20 (m, 3H). LCMS (M+H⁺) m/z: 494.4.

### Example 128: Preparation of N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (Compound 128)

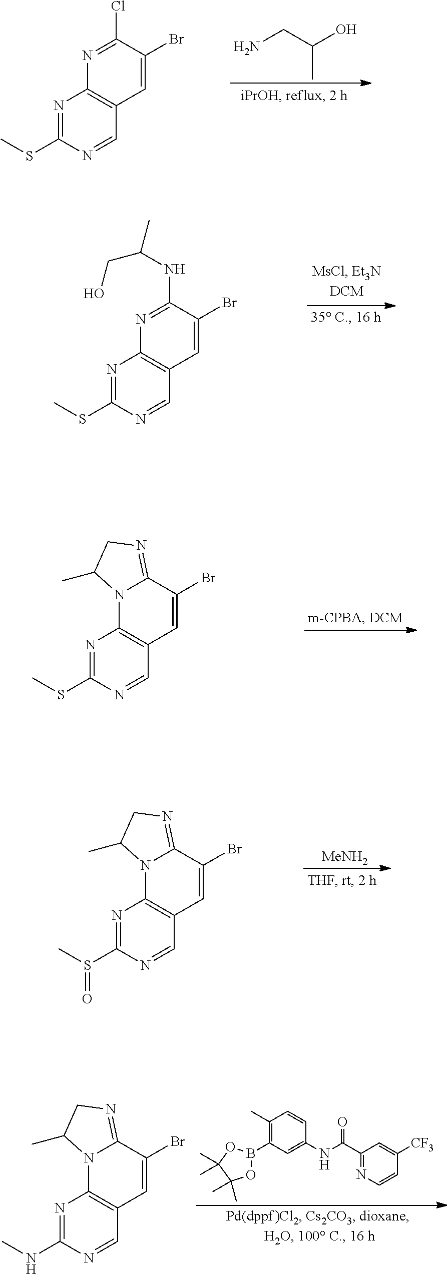

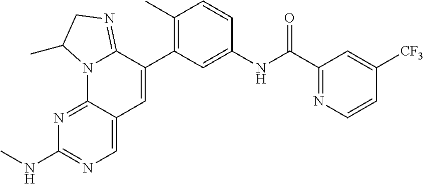 [SMILES: CNc1ncc2c(n1)N1C(=NCC1C)C(c1cc(NC(=O)c3cc(C(F)(F)F)ccn3)ccc1C)=C2]

### Step 1: Synthesis of 1-((6-bromo-2-(methylthio)pyrido[2,3-d]pyrimidin-7-yl)amino)propan-2-ol

A mixture of 6-bromo-7-chloro-2-(methylthio)pyrido[2,3-d]pyrimidine (1.0 g, 3.45 mmol), 1-aminopropan-2-ol (517 mg, 6.89 mmol) in iPrOH (15.0 mL) was stirred under reflux for 3 h. The reaction mixture was concentrated, diluted with water (20 mL). The resulting solid was filtered to afford 1-((6-bromo-2-(methylthio)pyrido[2,3-d]pyrimidin-7-yl)amino)propan-2-ol (1.16 g, 100% yield) as an off-white solid. LCMS (M+H⁺) m/z: 329.0 and 331.0.

### Step 2: Synthesis of 6-bromo-9-methyl-2-(methylthio)-8,9-dihydroimidazo[1′,2′:1,6]pyrido[2,3-d]pyrimidine

To a mixture of 1-((6-bromo-2-(methylthio)pyrido[2,3-d]pyrimidin-7-yl)amino)propan-2-ol (1.07 g, 3.27 mmol), TEA (1.98 g, 19.62 mmol) in DCM (20.0 mL), was added MsCl (1.49 g, 13.08 mmol). The resulting mixture was stirred at 35° C. for 16 h. The reaction mixture was concentrated, diluted with cooled water (50 mL) and extracted with EA (50 mL×3). The combined organic phase was washed with brine (100 mL), dried with Na₂SO₄, filtered and concentrated. The residue was purified by column chromatography (PE/EA=3/1) to afford 6-bromo-9-methyl-2-(methylthio)-8,9-dihydroimidazo[1′,2′:1,6]pyrido[2,3-d]pyrimidine (776 mg, 75% yield) as a yellow solid. LCMS (M+H⁺) m/z: 311.0 and 313.0.

### Step 3: Synthesis of 6-bromo-9-methyl-2-(methylsulfinyl)-8,9-dihydroimidazo[1′,2′:1,6]pyrido[2,3-d]pyrimidine

To a solution of 6-bromo-9-methyl-2-(methylthio)-8,9-dihydroimidazo[1′,2′:1,6]pyrido[2,3-d]pyrimidine (200 mg, 0.64 mmol) in dry DCM (8.0 mL) was added m-CPBA (294 mg, 1.28 mmol) at 0° C. The resulting mixture was stirred at 0° C. for 30 min. The reaction mixture was concentrated to afford crude 6-bromo-9-methyl-2-(methylsulfinyl)-8,9-dihydroimidazo[1′,2′:1,6]pyrido[2,3-d]pyrimidine (500 mg, 100% yield) as a yellow solid, which was used in the next step without purification. LCMS (M+H2O)⁺ m/z: 326.9 and 328.9.

### Step 4: Synthesis of 6-bromo-N,9-dimethyl-8,9-dihydroimidazo[1′,2′:1,6]pyrido[2,3-d]pyrimidin-2-amine

To a solution of crude 6-bromo-9-methyl-2-(methylsulfinyl)-8,9-dihydroimidazo[1′,2′:1,6]pyrido[2,3-d]pyrimidine (500 mg, 1.5 mmol) in THF (10.0 mL) was added MeNH₂ (2 M in THF, 3.0 mL, 3.0 mmol). The mixture was stirred at r.t. for 16 h and concentrated. Water was added and the mixture was extracted with EA (50 mL×3). The combined organic phase was washed with brine (100 mL), dried with Na₂SO₄, filtered and concentrated. The residue was added into EA (4.0 mL), stirred at r.t. for 3.0 h, filtered to afford 6-bromo-N,9-dimethyl-8,9-dihydroimidazo[1′,2′:1,6]pyrido[2,3-d]pyrimidin-2-amine (230 mg, 51% yield) as a yellow solid. LCMS (M+H+) m/z: 293.9 and 295.9.

### Step 5: N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide

A mixture of 6-bromo-N,9-dimethyl-8,9-dihydroimidazo[1′,2′:1,6]pyrido[2,3-d]pyrimidin-2-amine (230 mg, 0.78 mmol), N-(4-methyl-3-(4,4,5,5-tetramethyl-1,3,2-dioxaborolan-2-yl)phenyl)-4-(trifluoromethyl)picolinamide (276 mg, 0.78 mmol), Cs₂CO₃ (760 mg, 2.34 mmol) and Pd(dppf)Cl₂ (57 mg, 0.078 mmol) in dioxane (15.0 mL) and water (3 mL) was degassed and changed with N₂ three times and stirred at 100° C. for 16 h. The reaction mixture was diluted with water (80.0 mL), extracted with EA (50 mL×3). The combined organic phase was washed with brine (100 mL), dried with Na₂SO₄, filtered and concentrated. The residue was purified by Prep-HPLC (0.1% HCOOH) to afford N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (113.1 mg, 29% yield) as a yellow solid. ¹H NMR (400 MHz, DMSO-d₆): δ 10.75 (s, 1H), 9.03 (d, J=4.8 Hz, 1H), 8.33 (s, 1H), 8.27 (s, 1H), 8.15 (s, 1H), 8.09-8.08 (m, 1H), 7.82 (d, J=2.0 Hz, 2H), 7.79 (d, J=2.0 Hz, 1H), 7.77 (d, J=2.4 Hz, 1H), 7.52-7.40 (m, 1H), 7.25-7.17 (m, 1H), 4.74-4.63 (m, 1H), 4.05 (t, J=14.4 Hz, 1H), 3.50 (dd, J=15.2, 4.8 Hz, 1H), 2.85 (d, J=4.0 Hz, 3H), 2.20 (s, 3H), 1.48 (s, 3H). LCMS (M+H⁺) m/z: 494.3.

### Example 129 and 130: Preparation of (R)—N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (Compound 129) and (S)—N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (Compound 130)

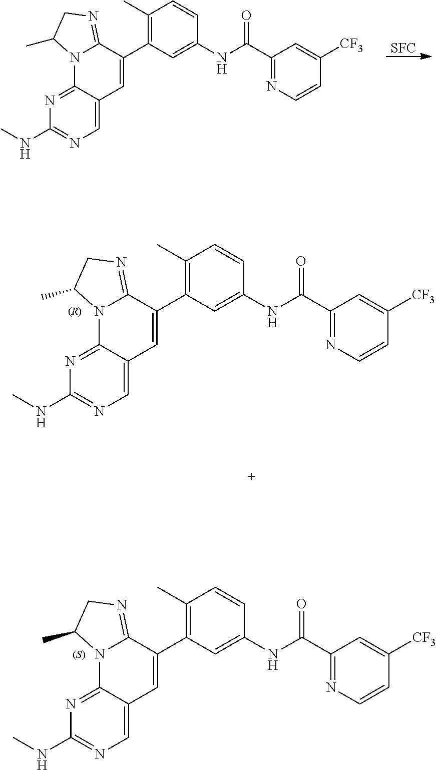

N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (60 mg) was separated by Chiral-HPLC to afford (R)—N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (Compound 129) (21 mg) and (S)—N-(4-methyl-3-(9-methyl-2-(methylamino)-8,9-dihydroimidazo[1′,2′:1,6]pyrido[2,3-d]pyrimidin-6-yl)phenyl)-4-(trifluoromethyl)picolinamide (Compound 130) (16 mg) as a yellow solid. Compound 129: ¹H NMR (400 MHz, DMSO-d₆): δ 10.74 (s, 1H), 9.025 (d, J=5.2 Hz, 1H), 8.33 (s, 1H), 8.23 (s, 1H), 8.09-8.08 (m, 1H), 7.81 (s, 1H), 7.78 (t, J=8.0 Hz, 1H), 7.40 (s, 1H), 7.23 (d, J=4.4 Hz, 1H), 4.67 (s, 1H), 4.06-4.01 (m, 2H), 3.94-3.89 (m, 2H), 2.20 (s, 3H), 1.45-1.23 (m, 3H). LCMS (M+H⁺) m/z: 494.4. Compound 130: ¹H NMR (400 MHz, DMSO-d₆): δ 10.74 (s, 1H), 9.03 (m, 1H), 8.34 (s, 1H), 8.23 (s, 1H), 8.09-8.08 (m, 1H), 7.81-7.78 (m, 2H), 7.41 (m, 1H), 7.41-7.35 (m, 1H), 7.23-7.21 (m, 1H), 7.11 (s, 1H), 4.68 (s, 1H), 4.08-4.02 (m, 1H), 3.51-3.48 (m, 1H), 2.20 (s, 3H), 1.46 (s, 3H). LCMS (M+H⁺) m/z: 494.4.

### Example 131: Preparation of N-(4-methyl-3-(2′-(methylamino)-8′H-spiro[cyclopentane-1,9′-imidazo[1′,2′:1,6]pyrido[2,3-d]pyrimidin]-6′-yl)phenyl)-4-(trifluoromethyl)picolinamide (Compound 131)

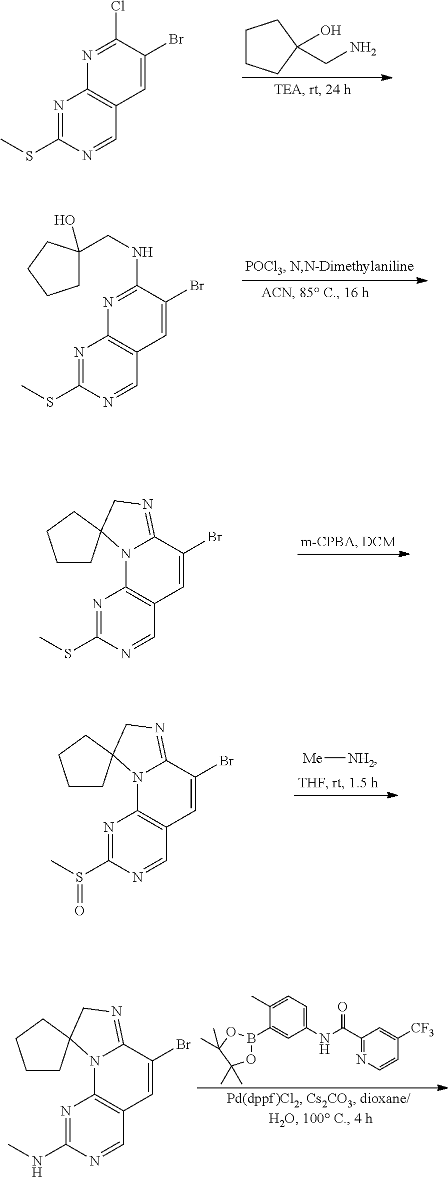

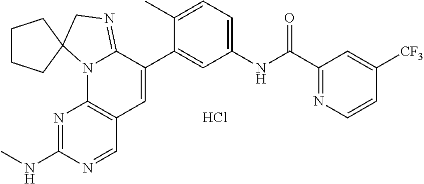 [SMILES: CNc1ncc2c(n1)N1C(=NCC13CCCC3)C(c1cc(NC(=O)c3cc(C(F)(F)F)ccn3)ccc1C)=C2 ; Cl]

### Step 1: Synthesis of 1-(((6-bromo-2-(methylthio)pyrido[2,3-d]pyrimidin-7-yl)amino)methyl)cyclopentan-1-ol

A mixture of 6-bromo-7-chloro-2-(methylthio)pyrido[2,3-d]pyrimidine (1.43 g, 4.92 mmol), 1-(aminomethyl)cyclopentan-1-ol (0.85 g, 7.38 mmol) in TEA (40.0 mL) was stirred at r.t. for 24 h. The reaction mixture was concentrated, diluted with water (100.0 mL) and extracted with DCM (100 mL×3). The combined organic phase was washed with brine (100 mL), dried with Na₂SO₄, filtered and concentrated. The residue was purified by column chromatography (PE/EA=1/1) to afford 1-(((6-bromo-2-(methylthio)pyrido[2,3-d]pyrimidin-7-yl)amino)methyl)cyclopentan-1-ol (1.79 g, 98% yield) as an off-white solid. LCMS (M+H⁺) m/z: 368.8 and 370.8.

### Step 2: Synthesis of 6′-bromo-2′-(methylthio)-8′H-spiro[cyclopentane-1,9′-imidazo[1′,2′:1,6]pyrido[2,3-d]pyrimidine]

To a mixture of 1-(((6-bromo-2-(methylthio)pyrido[2,3-d]pyrimidin-7-yl)amino)methyl)cyclopentan-1-ol (500 mg, 1.35 mmol), N,N-Dimethylaniline (164 mg, 1.35 mmol) in ACN (40.0 mL) was added POCl₃ (1.66 g, 10.84 mmol). The resulting mixture was stirred at 85° C. for 16 h. The reaction mixture was concentrated, diluted with cooled water (50.0 mL) and extracted with EA (50 mL×3). The combined organic phase was washed with brine (100 mL), dried with Na₂SO₄, filtered and concentrated. The residue was purified by column chromatography (PE/EA=3/1) to afford 6′-bromo-2′-(methylthio)-8′H-spiro[cyclopentane-1,9′-imidazo[1′,2′:1,6]pyrido[2,3-d]pyrimidine] (255 mg, 47% yield) as a yellow solid. LCMS (M+H+) m/z: 351.0 and 353.0.

### Step 3: Synthesis of 6′-bromo-2′-(methylsulfinyl)-8′H-spiro[cyclopentane-1,9′-imidazo[1′,2′:1,6]pyrido[2,3-d]pyrimidine]

To a solution of 6′-bromo-2′-(methylthio)-8′H-spiro[cyclopentane-1,9′-imidazo[1′,2′:1,6]pyrido[2,3-d]pyrimidine] (175 mg, 0.5 mmol) in dry DCM (5.0 mL) was added m-CPBA (229 mg, 1.0 mmol) at 0° C. The resulting mixture was stirred at 0° C. for 30 min. The reaction mixture was concentrated to afford crude 6′-bromo-2′-(methylsulfinyl)-8′H-spiro[cyclopentane-1,9′-imidazo[1′,2′:1,6]pyrido[2,3-d]pyrimidine] (400 mg, crude) as a yellow solid, which was used in the next step without purification. LCMS (M+H⁺) m/z: 366.9 and 368.9.

### Step 4: Synthesis of 6′-bromo-N-methyl-8′H-spiro[cyclopentane-1,9′-imidazo[1′,2′:1,6]pyrido[2,3-d]pyrimidin]-2′-amine

To a solution of crude 6′-bromo-2′-(methylsulfinyl)-8′H-spiro[cyclopentane-1,9′-imidazo[1′,2′:1,6]pyrido[2,3-d]pyrimidine] (400 mg, 0.50 mmol) in THF (5.0 mL) was added MeNH₂ (2 M in THF, 1.5 mL, 3.0 mmol). The mixture was stirred at r.t. for 1.5 h and concentrated. Water was added and the mixture was extracted with EA (50 mL×3). The combined organic phase was washed with brine (100 mL), dried with Na₂SO₄, filtered and concentrated. The residue was added into EA (4.0 mL), stirred at r.t. for 3 h, filtered to afford 6′-bromo-N-methyl-8′H-spiro[cyclopentane-1,9′-imidazo[1′,2′:1,6]pyrido[2,3-d]pyrimidin]-2′-amine (134 mg, 63% yield) as a pale yellow solid. LCMS (M+H+) m/z: 334.0 and 336.0.

### Step 5: Synthesis of N-(4-methyl-3-(2′-(methylamino)-8′H-spiro[cyclopentane-1,9′-imidazo[1′,2′:1,6]pyrido[2,3-d]pyrimidin]-6′-yl)phenyl)-4-(trifluoromethyl)picolinamide hydrogen chloride

A mixture of 6′-bromo-N-methyl-8′H-spiro[cyclopentane-1,9′-imidazo[1′,2′:1,6]pyrido[2,3-d]pyrimidin]-2′-amine (134 mg, 0.4 mmol), N-(4-methyl-3-(4,4,5,5-tetramethyl-1,3,2-dioxaborolan-2-yl)phenyl)-4-(trifluoromethyl)picolinamide (195 mg, 0.48 mmol), Cs₂CO₃ (392 mg, 1.2 mmol) and Pd(dppf)Cl₂ (29 mg, 0.04 mmol) in dioxane (8.0 mL) and water (0.8 mL) was degassed and charged with N₂ three times and stirred at 100° C. for 4 h. The reaction mixture was diluted with water (80.0 mL), extracted with EA (50 mL×3). The combined organic phase was washed with brine (100 mL), dried with Na₂SO₄, filtered and concentrated. The residue was purified by Prep-HPLC (0.1% HCl) to afford N-(4-methyl-3-(2′-(methylamino)-8′H-spiro[cyclopentane-1,9′-imidazo[1′,2′:1,6]pyrido[2,3-d]pyrimidin]-6′-yl)phenyl)-4-(trifluoromethyl)picolinamide hydrogen chloride (39.6 mg, 17% yield) as a yellow solid. ¹H NMR (400 MHz, DMSO-d₆): δ 10.89 (s, 1H), 9.89-9.38 (m, 1H), 9.05 (d, J=4.4 Hz, 1H), 8.98-8.90 (m, 2H), 8.55 (s, 1H), 8.34 (s, 1H), 8.13 (d, J=7.6 Hz, 2H), 8.01 (s, 1H), 7.91 (d, J=8.0 Hz, 1H), 7.41 (d, J=8.0 Hz, 1H), 3.93 (s, 2H), 3.04-2.94 (m, 5H), 2.21 (s, 3H), 2.03-1.97 (m, 4H), 1.73 (s, 2H). LCMS (M+H⁺) m/z: 534.4.

### Example 132: Preparation of N-(4-methyl-3-(8-(methylamino)-1,2-dihydroimidazo[1,2-a][1,6]naphthyridin-4-yl)phenyl)-4-(trifluoromethyl)picolinamide (Compound 132)

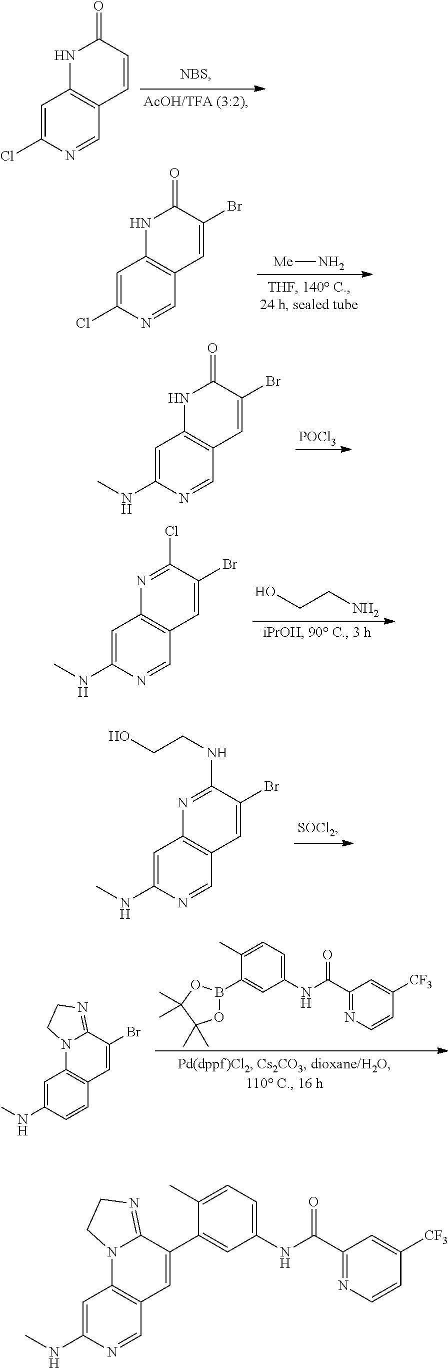

### Step 1: Synthesis of 3-bromo-7-chloro-1,6-naphthyridin-2(1H)-one

The mixture of 7-chloro-1,6-naphthyridin-2(1H)-one (400 mg, 2.21 mmol) in AcOH (6 mL) and TFA (4 mL) was stirred at room temperature for 20 mins. NBS (439 mg, 2.44 mmol) was added and the mixture was stirred at 70° C. for 16 hours. The reaction mixture was diluted with sat. NaHCO₃ (100 mL) and extracted with DCM (50 mL×2). The combined organic layers were washed with brine (60 mL) and dried over Na₂SO₄, filtered, concentrated to afford 3-bromo-7-chloro-1,6-naphthyridin-2(1H)-one (500 mg, crude) as a yellow solid. LCMS (M+H⁺) m/z: 260.8.

### Step 2: Synthesis of 3-bromo-7-(methylamino)-1,6-naphthyridin-2(1H)-one

To a mixture of 3-bromo-7-chloro-1,6-naphthyridin-2(1H)-one (700 mg, 2.70 mmol) in THF (10 mL) was added methylamine (15 mL, 2 M in THF). The solution was stirred at 140° C. for 24 hours. The reaction mixture was concentrated and the residue was purified by Prep-HPLC (0.1% NH3·H2O) to afford 3-bromo-7-(methylamino)-1,6-naphthyridin-2(1H)-one (250 mg, 36% yield) as a white solid. LCMS (M+H⁺) m/z: 254.0.

### Step 3: Synthesis of 3-bromo-2-chloro-N-methyl-1,6-naphthyridin-7-amine

The mixture of 3-bromo-7-(methylamino)-1,6-naphthyridin-2(1H)-one (180 mg, 0.71 mmol) in POCl₃ (10 mL) was stirred at 95° C. for 16 hours. The reaction mixture was concentrated then quenched with sat NaHCO₃ (30 mL) and extracted with DCM (30 mL×3). The combined organic layers were washed with brine (50 mL) and dried over Na₂SO₄, filtered, concentrated to give 3-bromo-2-chloro-N-methyl-1,6-naphthyridin-7-amine (250 mg, crude) as a yellow oil. LCMS (M+H⁺) m/z: 274.1.

### Step 4: Synthesis of 2-((3-bromo-7-(methylamino)-1,6-naphthyridin-2-yl)amino)ethan-1-ol

The mixture of 3-bromo-2-chloro-N-methyl-1,6-naphthyridin-7-amine (250 mg, 0.90 mmol) 2-aminoethan-1-ol (84 mg, 1.4 mmol) in iPrOH (5 mL) was stirred at 90° C. for 16 hours. The reaction mixture was concentrated, then purified by silica column chromatography (EA:PE=5% to 80%) to afford 2-((3-bromo-7-(methylamino)-1,6-naphthyridin-2-yl)amino)ethan-1-ol (60 mg, 22% yield) as yellow oil. LCMS (M+H⁺) m/z: 299.0.

### Step 5: Synthesis of 4-bromo-N-methyl-1,2-dihydroimidazo[1,2-a][1,6]naphthyridin-8-amine

SOCl₂ (120 mg, 1.0 mmol) was added to a solution of 2-((3-bromo-7-(methylamino)-1,6-naphthyridin-2-yl)amino)ethan-1-ol (60 mg, 0.20 mmol) in CHCl₃ (3 mL). The reaction mixture was stirred at 70° C. for 6 hours. The mixture was quenched with sat NaHCO₃ (50 mL) and extracted with DCM (50 mL×3). The combined organic layers were washed with brine (100 mL) and dried over Na₂SO₄, filtered, concentrated to give the crude which was purified by silica column chromatography (EA/PE=5% to 80%) to give 4-bromo-N-methyl-1,2-dihydroimidazo[1,2-a][1,6]naphthyridin-8-amine (20 mg, 36% yield) as a yellow solid. LCMS (M+H⁺) m/z: 279.1

### Step 6: Synthesis of N-(4-methyl-3-(8-(methylamino)-1,2-dihydroimidazo[1,2-a][1,6]naphthyridin-4-yl)phenyl)-4-(trifluoromethyl)picolinamide

A mixture of 4-bromo-N-methyl-1,2-dihydroimidazo[1,2-a][1,6]naphthyridin-8-amine (15 mg, 0.05 mmol), N-(4-methyl-3-(4,4,5,5-tetramethyl-1,3,2-dioxaborolan-2-yl)phenyl)-4-(trifluoromethyl) picolinamide (32.7 mg, 0.08 mmol), Cs₂CO₃ (52 mg, 0.16 mmol) and Pd(dppf)Cl₂ (3.9 mg, 0.005 mmol) in dioxane:H₂O (10:1) (2 mL) was degassed and charged with N₂ three times, stirred at 110° C. for 16 hours. The reaction mixture was concentrated, diluted with 3M HCl (30 mL) and extracted with DCM (30 mL). The aqueous phase was adjusted to pH=10 with NH₃·H₂O and extracted with DCM (30 mL×3). The combined organic layers were washed with brine (60 mL) and dried over Na₂SO₄, filtered, concentrated to give the crude product, which was purified by Prep-TLC (DCM:MeOH=10:1), followed by Prep-HPLC (0.1% NH₃H₂O) to afford N-(4-methyl-3-(8-(methylamino)-1,2-dihydroimidazo[1,2-a][1,6]naphthyridin-4-yl)phenyl)-4-(trifluoromethyl)picolinamide (3.4 mg, 10% yield) as a yellow solid. ¹H NMR (400 MHz, CD₃OD-d₄): δ 8.96-8.95 (m, 1H), 8.42 (d, J=8.8 Hz, 2H), 7.92 (d, J=5.2 Hz, 1H), 7.84-7.83 (m, 1H), 7.80-7.76 (m, 2H), 7.39 (d, J=8.4 Hz, 1H), 6.10 (s, 1H), 4.45 (t, J=10.4 Hz, 2H), 4.08 (t, J=10.4 Hz, 2H), 2.99 (s, 3H), 2.27 (s, 3H). LCMS (M+H+) m/z: 479.2.

### Example 133: Preparation of N-(2-chloro-4-methyl-5-(2-(methylamino)-8,9-dihydroimidazo[1′,2′:1,6]pyrido[2,3-d]pyrimidin-6-yl)phenyl)-4-(trifluoromethyl)picolinamide (Compound 133)

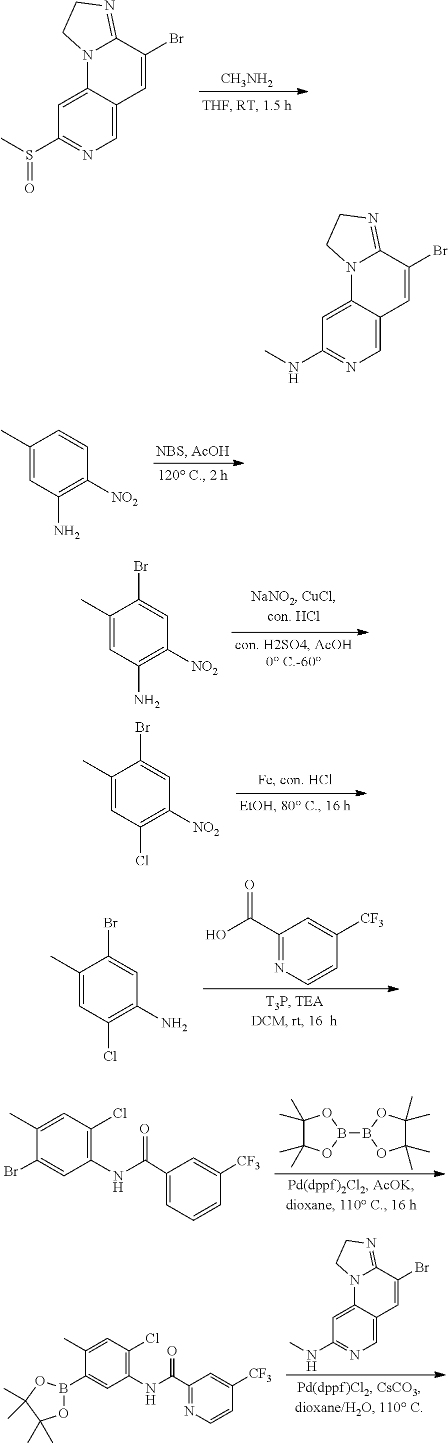

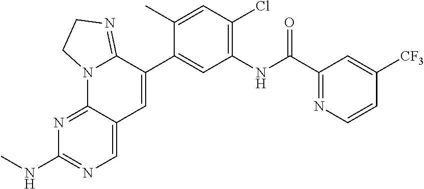 [SMILES: CNc1ncc2c(n1)N1CCN=C1C(c1cc(NC(=O)c3cc(C(F)(F)F)ccn3)c(Cl)cc1C)=C2]

The preparation of 6-bromo-2-(methylsulfinyl)-8,9-dihydroimidazo[1′,2′:1,6]pyrido[2,3-d]pyrimidine was described in Example 109

### Step 1: Synthesis of 6-bromo-N-methyl-8,9-dihydroimidazo[1′,2′:1,6]pyrido[2,3-d]pyrimidin-2-amine

To a mixture of 6-bromo-2-(methylsulfinyl)-8,9-dihydroimidazo[1′,2′:1,6]pyrido[2,3-d]pyrimidine (1.2 g, 3.85 mmol) in THF (20 mL) was added MeNH₂ (2.0 M in THF, 5.8 mL, 11.55 mmol) at RT. The reaction mixture was stirred at RT for 1 h. The reaction mixture was removed in vacuum to afford 6-bromo-N-methyl-8,9-dihydroimidazo[1′,2′:1,6]pyrido[2,3-d]pyrimidin-2-amine (710 mg, 75.4% yield) as a yellow solid. LCMS (M+H⁺) m/z: 280.0 and 282.0.

### Step 2: Synthesis of 4-bromo-5-methyl-2-nitroaniline

To a solution of 5-methyl-2-nitroaniline (3.0 g, 19.7 mmol) in AcOH (100 mL) were added NBS (3.58 g, 20.1 mmol). The mixture was stirred at 120° C. under N₂ for 1.5 h. The mixture was poured into water (300 mL), filtered to afford 4-bromo-5-methyl-2-nitroaniline (4.2 g, 93% yield) as a yellow solid.

### Step 3: Synthesis of 1-bromo-4-chloro-2-methyl-5-nitrobenzene

4-Bromo-5-methyl-2-nitroaniline (2.0 g, 8.66 mmol) in AcOH (20 mL) was slowly added into a solution of NaNO₂ (955 mg, 13.8 mmol) in conc. H₂SO₄ (10 mL) while maintained the temperature below 40° C. The mixture was stirred at rt for 30 mins. The resulting mixture was slowly added into a solution of CuCl (2.0 g, 20.7 mmol) in conc. HCl (25 mL). The reaction mixture was stirred at 60° C. for 2 h. Water was added, the resulting precipitate was filtered to afford 1-bromo-4-chloro-2-methyl-5-nitrobenzene (1.5 g, 70% yield) as a grey solid.

### Step 4: Synthesis of 5-bromo-2-chloro-4-methylaniline

To a solution of 1-bromo-4-chloro-2-methyl-5-nitrobenzene (800 g, 3.19 mmol) in EtOH (8 mL) and H₂O (2 mL) was added Fe powder (894 mg, 15.9 mmol) and conc. HCl (0.5 mL). The mixture was stirred at 80° C. for 16 h. The mixture was filtered and concentrated, purified on silica gel column chromatography (PE:EA=4:1) to afford 5-bromo-2-chloro-4-methylaniline (550 mg, 78% yield) as a yellow solid. LCMS (M+H⁺) m/z: 219.9.

### Step 5: Synthesis of N-(5-bromo-2-chloro-4-methylphenyl)-4-(trifluoromethyl)picolinamide

To a solution of 5-bromo-2-chloro-4-methylaniline (420 mg, 1.90 mmol) in DCM (10 mL) was added 4-(trifluoromethyl)picolinic acid (436 mg, 2.28 mmol), T3P (1.2 g, 3.81 mmol) and TEA (578 mg, 5.72 mmol). The mixture was stirred at rt under N2 for 2 h. The mixture was diluted with water (30 mL) and then extracted with DCM (30 mL×3). The residue was purified on silica gel column chromatography (PE:EA=10:1) to afford N-(5-bromo-2-chloro-4-methylphenyl)-4-(trifluoromethyl)picolinamide (550 mg, 73% yield) as a white solid. LCMS (M+H+) m/z: 395.0.

### Step 6: Synthesis of N-(2-chloro-4-methyl-5-(4,4,5,5-tetramethyl-1,3,2-dioxaborolan-2-yl)phenyl)-4-(trifluoromethyl)picolinamide

To a solution of N-(5-bromo-2-chloro-4-methylphenyl)-4-(trifluoromethyl)picolinamide (150 mg, 0.38 mmol) in 1,4-dioxane (5 mL) was added 4,4,4′,4′,5,5,5′,5′-octamethyl-2,2′-bi(1,3,2-dioxaborolane) (193 mg, 0.76 mmol), Pd(dppf)Cl₂ (27 mg, 0.038 mmol) and AcOK (112 mg, 1.14 mmol). The mixture was stirred at 110° C. under N2 for 16 h. The mixture was concentrated and purified on silica gel column chromatography (PE:EA=10:1) to afford N-(2-chloro-4-methyl-5-(4,4,5,5-tetramethyl-1,3,2-dioxaborolan-2-yl)phenyl)-4-(trifluoromethyl)picolinamide (110 mg, 66% yield) as a yellow solid. LCMS (M+H⁺) m/z: 441.1.

### Step 7: Synthesis of N-(2-chloro-4-methyl-5-(2-(methylamino)-8,9-dihydroimidazo[1′,2′:1,6]pyrido[2,3-d]pyrimidin-6-yl)phenyl)-4-(trifluoromethyl)picolinamide

To a solution of N-(2-chloro-4-methyl-5-(4,4,5,5-tetramethyl-1,3,2-dioxaborolan-2-yl)phenyl)-4-(trifluoromethyl)picolinamide (110 mg, 0.25 mmol) in 1,4-dioxane (5 mL) and H₂O (0.5 mL) was added 6-bromo-N-methyl-8,9-dihydroimidazo[1′,2′:1,6]pyrido[2,3-d]pyrimidin-2-amine (77 mg, 0.27 mmol), Pd(dppf)Cl₂ (18 mg, 0.025 mmol) and Cs2CO3 (224 mg, 0.75 mmol). The mixture was stirred at 110° C. under N2 for 16 h. The mixture was purified by prep-HPLC (0.1%/HCl/CH3CN/H2O) to afford N-(2-chloro-4-methyl-5-(2-(methylamino)-8,9-dihydroimidazo[1′,2′:1,6]pyrido[2,3-d]pyrimidin-6-yl)phenyl)-4-(trifluoromethyl)picolinamide (7.4 mg, 6% yield) as a grey solid. ¹H NMR (400 MHz, DMSO-d₆): δ 10.65 (s, 1H), 9.92 (s, 1H), 9.83 (s, 1H), 9.09 (d, J=5.2 Hz, 1H), 8.98 (s, 1H), 8.89 (s, 1H), 8.56 (q, J=4.8 Hz, 1H), 8.36 (s, 1H), 8.30 (s, 1H), 8.19 (d, J=5.2 Hz, 1H), 8.17 (s, 1H), 7.70 (s, 1H), 4.67-4.53 (m, 2H), 4.06-3.98 (m, 2H), 2.97 (d, J=4.8 Hz, 3H), 2.22 (s, 3H). LCMS (M+H⁺) m/z: 514.0.

### Example 134: Preparation of N-(2-fluoro-4-methyl-5-(2-(methylamino)-8,9-dihydroimidazo[1′,2′:1,6]pyrido[2,3-d]pyrimidin-6-yl)phenyl)-2-(3-(trifluoromethyl)phenyl)acetamide (Compound 134)

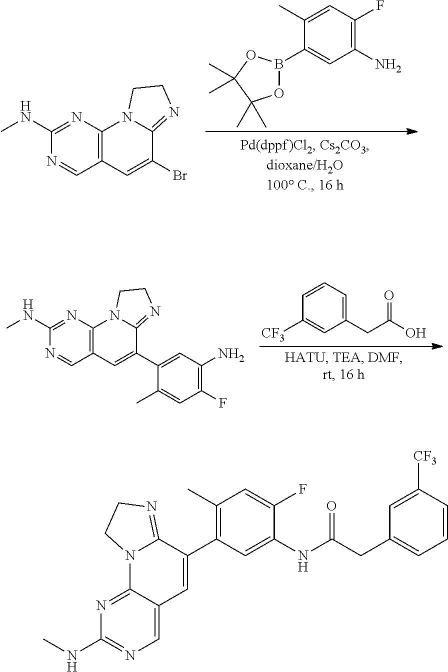

### Step 1: Synthesis of 6-(5-amino-4-fluoro-2-methylphenyl)-N-methyl-8,9-dihydroimidazo[1′,2′:1,6]pyrido[2,3-d]pyrimidin-2-amine

To a solution of 6-bromo-N-methyl-8,9-dihydroimidazo[1′,2′:1,6]pyrido[2,3-d]pyrimidin-2-amine (600 mg, 2.14 mmol) in dioxane (12 mL) and H₂O (1 mL) was added 2-fluoro-4-methyl-5-(4,4,5,5-tetramethyl-1,3,2-dioxaborolan-2-yl)aniline (591 mg, 2.35 mmol), Pd(dppf)Cl₂ (156 mg, 0.21 mmol) and Cs₂CO₃ (2.0 g, 6.42 mmol). The mixture was stirred at 110° C. under N₂ for 16 h. The mixture was concentrated, purified by column chromatography (DCM:MeOH=10:1) to afford 6-(5-amino-4-fluoro-2-methylphenyl)-N-methyl-8,9-dihydroimidazo[1′,2′:1,6]pyrido[2,3-d]pyrimidin-2-amine (320 mg, 46%, 0.99 mmol) as a grey solid. LCMS (M+H⁺) m/z: 325.2.

### Step 2: Synthesis of N-(2-fluoro-4-methyl-5-(2-(methylamino)-8,9-dihydroimidazo[1′,2′:1,6]pyrido[2,3-d]pyrimidin-6-yl)phenyl)-2-(3-(trifluoromethyl)phenyl)acetamide

To a solution of 6-(5-amino-4-fluoro-2-methylphenyl)-N-methyl-8,9-dihydroimidazo[1′,2′:1,6]pyrido[2,3-d]pyrimidin-2-amine (330 mg, 0.32 mmol) in DMF (3 mL) was added 2-(3-(trifluoromethyl)phenyl)acetic acid (27 mg, 0.13 mmol), HATU (70 mg, 0.18 mmol) and TEA (37 mg, 0.37 mmol). The mixture was stirred at rt for 16 hours. The mixture was purified by prep-HPLC (0.1%/HCl/CH3CN/H2O) to afford N-(2-fluoro-4-methyl-5-(2-(methylamino)-8,9-dihydroimidazo[1′,2′:1,6]pyrido[2,3-d]pyrimidin-6-yl)phenyl)-2-(3-(trifluoromethyl)phenyl)acetamide (28.3 mg, 46% yield) as a yellow solid. ¹H NMR (400 MHz, DMSO-d₆): δ 10.27 (s, 1H), 9.70 (s, 1H), 8.84 (s, 1H), 8.52 (q, J=4.8 Hz, 1H), 8.06 (s, 1H), 7.86 (d, J=8.0 Hz, 1H), 7.72 (s, 1H), 7.65-7.55 (m, 3H), 7.33 (d, J=12.0 Hz, 1H), 4.64-4.51 (m, 2H), 4.01-3.96 (m, 2H), 3.89 (s, 2H), 2.95 (d, J=4.8 Hz, 3H), 2.16 (s, 3H). LCMS (M+H⁺) m/z: 511.4.

### Example 135: Preparation of N-(4-fluoro-3-(2-(methylamino)-8,9-dihydroimidazo[1′,2′:1,6]pyrido[2,3-d]pyrimidin-6-yl)phenyl)-4-(trifluoromethyl)picolinamide (Compound 135)

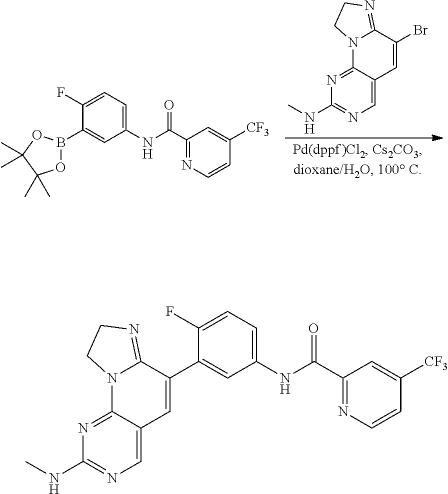 [SMILES: CC1(C)OB(c2cc(NC(=O)c3cc(C(F)(F)F)ccn3)ccc2F)OC1(C)C ; CNc1ncc2c(n1)N1CCN=C1C(Br)=C2 ; CNc1ncc2c(n1)N1CCN=C1C(c1cc(NC(=O)c3cc(C(F)(F)F)ccn3)ccc1F)=C2]

The preparation of 6-bromo-N-methyl-8,9-dihydroimidazo[1′,2′:1,6]pyrido[2,3-d]pyrimidin-2-amine and N-(4-fluoro-3-(4,4,5,5-tetramethyl-1,3,2-dioxaborolan-2-yl)phenyl)-4-(trifluoromethyl)picolinamide was described in Example 121 and Example 133.

### Step 1: Synthesis of N-(4-fluoro-3-(2-(methylamino)-8,9-dihydroimidazo[1′,2′:1,6]pyrido[2,3-d]pyrimidin-6-yl)phenyl)-4-(trifluoromethyl)picolinamide

To a mixture of N-(4-fluoro-3-(4,4,5,5-tetramethyl-1,3,2-dioxaborolan-2-yl)phenyl)-4-(trifluoromethyl) picolinamide (267 mg, 0.65 mmol) in dioxane/H₂O (15 mL/1.5 mL) was added 6-bromo-N-methyl-8,9-dihydroimidazo[1′,2′:1,6]pyrido[2,3-d]pyrimidin-2-amine (165 mg, 0.59 mmol), Pd(dppf)Cl₂ (44 mg, 0.06 mmol), Cs₂CO₃ (577 mg, 1.77 mmol), the mixture was degassed three times and charged with N₂, stirred at 100° C. for 3 hrs. The reaction mixture was concentrated in vacuum and H₂O (50.0 mL) was added. The reaction mixture was extracted with EA (50 mL×3). The combined organic phases were washed with brine (100.0 mL), dried over Na₂SO₄, filtered and concentrated. The residue was triturated with EA (4 mL) to afford N-(4-fluoro-3-(2-(methylamino)-8,9-dihydroimidazo[1′,2′:1,6]pyrido[2,3-d]pyrimidin-6-yl)phenyl)-4-(trifluoromethyl)picolinamide (108.9 mg, 38% yield) as a yellow solid. ¹H NMR (400 MHz, DMSO-d₆): δ 10.90 (s, 1H), 9.04 (d, J=4.8 Hz, 1H), 8.35 (s, 1H), 8.28-8.22 (m, 1H), 8.14-8.09 (m, 2H), 7.91-7.87 (m, 1H), 7.48 (s, 1H), 7.32 (s, 1H), 7.26 (t, J=9.6 Hz, 1H), 4.05-3.90 (m, 4H), 2.85 (s, 3H). LCMS (M+H⁺) m/z: 484.3.

### Example 136: Preparation of N-(4-chloro-3-(2-(methylamino)-8,9-dihydroimidazo[1′,2′:1,6]pyrido[2,3-d]pyrimidin-6-yl)phenyl)-4-(trifluoromethyl)picolinamide (Compound 136)

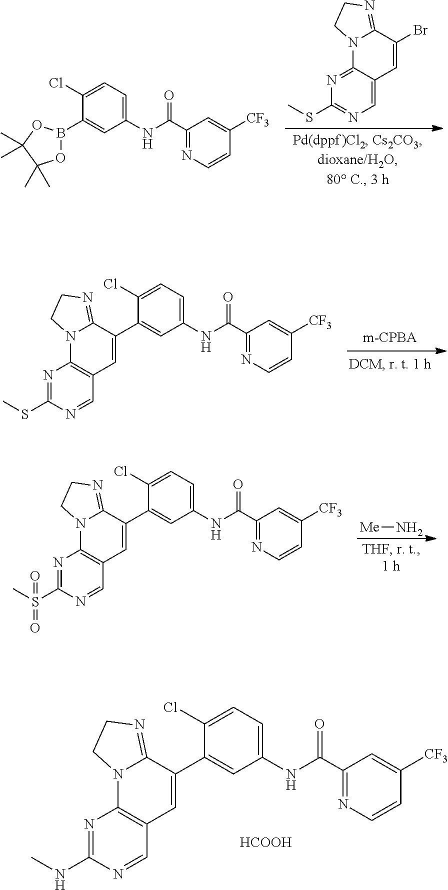

The preparation of N-(4-chloro-3-(4,4,5,5-tetramethyl-1,3,2-dioxaborolan-2-yl)phenyl)-4-(trifluoromethyl)picolinamide was described in Example 123.

### Step 1: Synthesis of N-(4-Chloro-3-(2-(methylthio)-8,9-dihydroimidazo[1′,2′:1,6]pyrido[2,3-d]pyrimidin-6-yl)phenyl)-4-(trifluoromethyl)picolinamide

Pd(dppf)Cl₂ (87.8 mg, 0.12 mmol) was added to a mixture of N-(4-chloro-3-(4,4,5,5-tetramethyl-1,3,2-dioxaborolan-2-yl)phenyl)-4-(trifluoromethyl)picolinamide (500 mg, 1.17 mmol), 6-bromo-2-(methylthio)-8,9-dihydroimidazo[1′,2′:1,6]pyrido[2,3-d]pyrimidine (348 mg, 1.17 mmol), Cs₂CO₃ (1.14 g, 3.51 mmol) in Dioxane/H₂O (5:1) (12 mL) under N₂. The reaction mixture was stirred at 80° C. for 3 hours. The reaction mixture was concentrated and purified by silica column chromatography (EA/PE=0% to 80%) to afford N-(4-Chloro-3-(2-(methylthio)-8,9-dihydroimidazo[1′,2′:1,6]pyrido[2,3-d]pyrimidin-6-yl)phenyl)-4-(trifluoromethyl)picolinamide (260 mg, 43% yield) as a yellow solid. LCMS (M+H⁺) m/z: 517.0.

### Step 2: Synthesis of N-(4-Chloro-3-(2-(methylsulfonyl)-8,9-dihydroimidazo[1′,2′:1,6]pyrido[2,3-d]pyrimidin-6-yl)phenyl)-4-(trifluoromethyl)picolinamide

m-CPBA (153 mg, 0.89 mmol) was added to the mixture of N-(4-chloro-3-(2-(methylthio)-8,9-dihydroimidazo[1′,2′:1,6]pyrido[2,3-d]pyrimidin-6-yl)phenyl)-4-(trifluoromethyl)picolinamide (230 mg, 0.44 mmol) in DCM (5 mL) at 0° C. The reaction mixture was stirred at room temperature for 1 hour, concentrated to give N-(4-Chloro-3-(2-(methylsulfonyl)-8,9-dihydroimidazo[1′,2′:1,6]pyrido[2,3-d]pyrimidin-6-yl)phenyl)-4-(trifluoromethyl)picolinamide (244 mg, crude) as a yield solid, which was used to the next step without further purification. LCMS (M+H⁺) m/z: 533.1 and 549.1.

### Step 3: Synthesis of N-(4-Chloro-3-(2-(methylamino)-8,9-dihydroimidazo[1′,2′:1,6]pyrido[2,3-d]pyrimidin-6-yl)phenyl)-4-(trifluoromethyl)picolinamide

Me-NH₂ (2 mL) was added to a solution of N-(4-chloro-3-(2-(methylsulfonyl)-8,9-dihydroimidazo[1′,2′:1,6]pyrido[2,3-d]pyrimidin-6-yl)phenyl)-4-(trifluoromethyl)picolinamide (244 mg, 0.44 mmol) in THF (5 mL). The reaction mixture was stirred at room-temperature for 1 hour. The mixture was quenched with water (30 mL) and extracted with DCM (30 mL×3). The combined organic layers was washed with brine (50 mL) and dried over Na₂SO₄, filtered, concentrated to give the crude which was purified by Prep-HPLC (0.1% HCOOH) to give N-(4-chloro-3-(2-(methylamino)-8,9-dihydroimidazo[1′,2′:1,6]pyrido[2,3-d]pyrimidin-6-yl)phenyl)-4-(trifluoromethyl)picolinamide formate (41 mg, 16.9% yield) as a white solid. ¹H NMR (400 MHz, DMSO-d₆): δ 10.98 (s, 1H), 9.04 (d, J=4.8 Hz, 1H), 8.34 (s, 1H), 8.29-8.26 (m, 1H), 8.21 (s, 1H), 8.10 (d, J=4.8 Hz, 1H), 8.07 (s, 1H), 7.93 (dd, J=2.4 Hz, 12.8 Hz, 1H), 7.53-7.51 (m, 2H), 7.28 (s, 1H), 4.10-4.01 (m, 2H), 3.94-3.82 (m, 2H), 2.86 (s, 3H). LCMS (M+H⁺) m/z: 500.3.

### Example 137: Preparation of N-(4-chloro-3-(2-(oxetan-3-ylamino)-8,9-dihydroimidazo[1′,2′:1,6]pyrido[2,3-d]pyrimidin-6-yl)phenyl)-4-(trifluoromethyl)picolinamide (Compound 137)

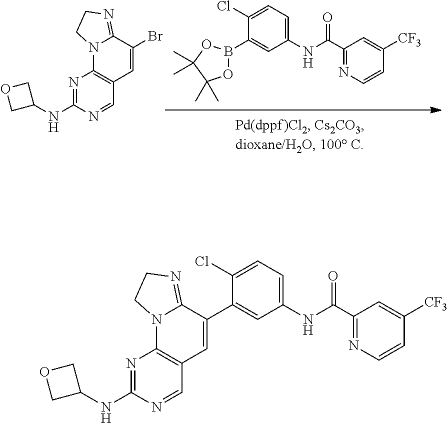

The preparation of 6-bromo-N-(oxetan-3-yl)-8,9-dihydroimidazo[1′,2′:1,6]pyrido[2,3-d]pyrimidin-2-amine was described in Example 114.

### Step 1: Synthesis of N-(4-chloro-3-(2-(oxetan-3-ylamino)-8,9-dihydroimidazo[1′,2′:1,6]pyrido[2,3-d]pyrimidin-6-yl)phenyl)-4-(trifluoromethyl)picolinamide

To a solution of 6-bromo-N-(oxetan-3-yl)-8,9-dihydroimidazo[1′,2′:1,6]pyrido[2,3-d]pyrimidin-2-amine (366 mg, 0.67 mmol) in dioxane/H₂O (15 mL/3 mL) was added N-(4-chloro-3-(4,4,5,5-tetramethyl-1,3,2-dioxaborolan-2-yl)phenyl)-4-(trifluoromethyl)picolinamide (343 mg, 0.80 mmol), Pd(dppf)Cl₂ (49 mg, 0.067 mmol), Cs₂CO₃ (653 mg, 2.01 mmol). The mixture was stirred for 16 h at 100° C. under N₂. Water (100 mL) was added to the reaction mixture. The mixture was extracted with EA (30 mL×3). The combined extracts were washed with brine (20 mL×2), dried over Na₂SO₄, concentrated under vacuum. The residue was triturated with MeOH and filtered to afford N-(4-chloro-3-(2-(oxetan-3-ylamino)-8,9-dihydroimidazo[1′,2′:1,6]pyrido[2,3-d]pyrimidin-6-yl)phenyl)-4-(trifluoromethyl)picolinamide (37.2 mg, 10% yield) as a yellow solid. ¹H NMR (400 MHz, DMSO-d₆): δ 10.98 (s, 1H), 9.03 (d, J=4.8 Hz, 1H), 8.34-8.28 (m, 3H), 8.10-8.05 (m, 2H), 7.94-7.91 (m, 1H), 7.51 (d, J=8.8 Hz, 1H), 7.23 (s, 1H), 4.95 (s, 1H), 4.78-4.75 (m, 2H), 4.56-4.53 (m, 2H), 4.04-4.00 (m, 2H), 3.93-3.89 (m, 2H). LCMS (M+H⁺) m/z: 542.4.

### Example 138: Preparation of N-(4-chloro-3-(2-((tetrahydro-2H-pyran-4-yl)amino)-8,9-dihydroimidazo[1′,2′:1,6]pyrido[2,3-d]pyrimidin-6-yl)phenyl)-4-(trifluoromethyl)picolinamide (Compound 138)

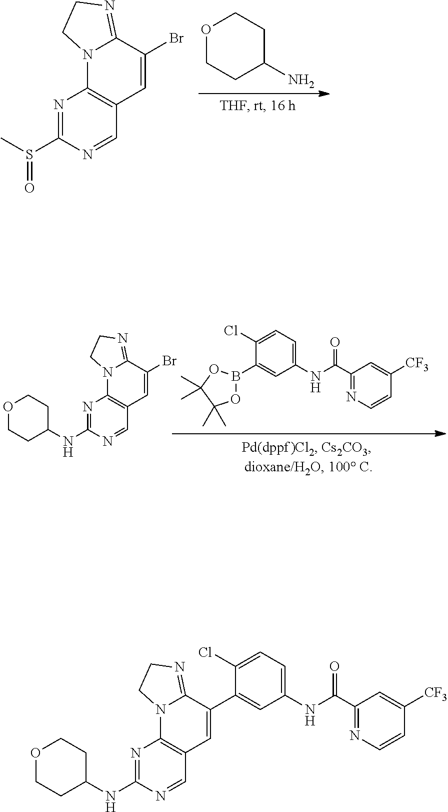

The preparation of 6-bromo-2-(methylthio)-8,9-dihydroimidazo[1′,2′:1,6]pyrido[2,3-d]pyrimidine was described in Example 26.

### Step 1: Synthesis of 6-bromo-N-(tetrahydro-2H-pyran-4-yl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (200 mg, 0.63 mmol) in THF (15 mL) was added tetrahydro-2H-pyran-4-amine (252 mg, 2.5 mmol). The reaction mixture was stirred at rt for 16 h. H₂O (20 mL) was added, the reaction mixture was extracted with EA (20 mL×3). The combined organic layers were dried over Na₂SO₄, concentrated under vacuum to afford 6-bromo-N-(tetrahydro-2H-pyran-4-yl)-8,9-dihydroimidazo[1′,2′:1,6]pyrido[2,3-d]pyrimidin-2-amine (237 mg, crude). LCMS (M+H⁺) m/z: 350.0 and 352.0.

### Step 2: Synthesis of N-(4-chloro-3-(2-((tetrahydro-2H-pyran-4-yl)amino)-8,9-dihydroimidazo[1′,2′:1,6]pyrido[2,3-d]pyrimidin-6-yl)phenyl)-4-(trifluoromethyl)picolinamide

To a solution of 6-bromo-N-(tetrahydro-2H-pyran-4-yl)-8,9-dihydroimidazo[1′,2′:1,6]pyrido[2,3-d]pyrimidin-2-amine (237 mg, 0.67 mmol) in dioxane/H₂O (15 mL/5 mL) was added N-(4-chloro-3-(4,4,5,5-tetramethyl-1,3,2-dioxaborolan-2-yl)phenyl)-4-(trifluoromethyl)picolinamide (346 mg, 0.81 mmol), Pd(dppf)Cl₂ (49 mg, 0.067 mmol), Cs₂CO₃ (653 mg, 2.01 mmol). The mixture was stirred for 16 h at 100° C. under N₂. H₂O (100 mL) was added, the mixture was extracted with EA (30 mL×3). The combined organic layers were washed with brine (20 mL×2), dried over Na₂SO₄, concentrated under vacuum. The crude was triturated with MeOH and filtered to afford N-(4-chloro-3-(2-((tetrahydro-2H-pyran-4-yl)amino)-8,9-dihydroimidazo[1′,2′:1,6]pyrido[2,3-d]pyrimidin-6-yl)phenyl)-4-(trifluoromethyl)picolinamide (14.7 mg, 4% yield) as a yellow solid. LCMS (M+H⁺) m/z: 570.4. ¹H NMR (400 MHz, DMSO-d₆): δ 9.04 (d, J=5.2 Hz, 1H), 8.38-8.34 (m, 2H), 8.18 (s, 1H), 8.09 (d, J=4.4 Hz, 1H), 8.05-8.04 (m, 1H), 7.93 (d, J=8.4 Hz, 1H), 7.55 (d, J=8.4 Hz, 1H), 7.46-7.36 (m, 1H), 4.19-4.07 (m, 3H), 3.96-3.88 (m, 4H), 3.43-3.33 (m, 2H), 1.91-1.80 (m, 2H), 1.57-1.56 (m, 2H).

### Example 139: Preparation of N-(2-fluoro-4-methyl-5-(2-(methylamino)-8,9-dihydroimidazo[1′,2′:1,6]pyrido[2,3-d]pyrimidin-6-yl)phenyl)-4-(trifluoromethyl)picolinamide (Compound 139)

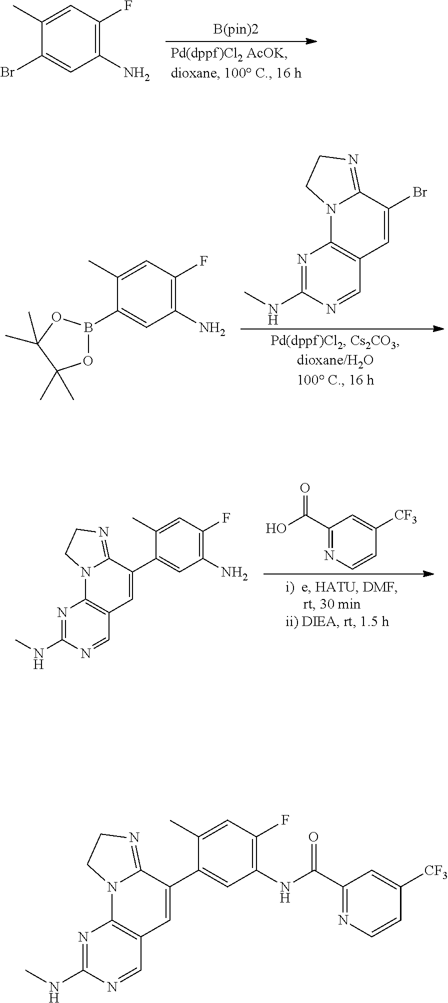

### Step 1: Synthesis of 2-fluoro-4-methyl-5-(4,4,5,5-tetramethyl-1,3,2-dioxaborolan-2-yl)aniline

A mixture of 5-bromo-2-fluoro-4-methylaniline (10 g, 49 mmol), bis(pinacolato)diboron (14.9 g, 58.8 mmol), KOAc (14.4 g, 147 mmol) and Pd(dppf)Cl₂ (3.59 g, 4.9 mmol) in dioxane (340.0 mL) was degassed and charged with N₂ for 3 times and stirred at 100° C. for 16.0 h. The reaction mixture was concentrated and purified by column chromatography (PE/EA=4/1) to afford 2-fluoro-4-methyl-5-(4,4,5,5-tetramethyl-1,3,2-dioxaborolan-2-yl)aniline (12.8 g, crude) as an off-white solid. LCMS (M+H⁺) m/z: 252.2.

### Step 2: Synthesis of 6-(5-amino-4-fluoro-2-methylphenyl)-N-methyl-8,9-dihydroimidazo[1′,2′:1,6]pyrido[2,3-d]pyrimidin-2-amine

A mixture of 2-fluoro-4-methyl-5-(4,4,5,5-tetramethyl-1,3,2-dioxaborolan-2-yl)aniline (2.9 g, 11.57 mmol), 6-bromo-N-methyl-8,9-dihydroimidazo[1′,2′:1,6]pyrido[2,3-d]pyrimidin-2-amine (2.7 g, 9.64 mmol), Cs2CO3 (7.86 g, 24.1 mmol) and Pd(dppf)Cl₂ (706 mg, 0.964 mmol) in dioxane (70.0 mL) and H₂O (7.0 mL) was degassed and charged with N₂ for 3 times and stirred at 100° C. for 16.0 h. The reaction mixture was concentrated and purified by column chromatography (DCM/MeOH=10/1) to afford 6-(5-amino-4-fluoro-2-methylphenyl)-N-methyl-8,9-dihydroimidazo[1′,2′:1,6]pyrido[2,3-d]pyrimidin-2-amine (2.01 g, 64.3% yield) as a brown solid. LCMS (M+H⁺) m/z: 325.1.

### Step 3: N-(2-fluoro-4-methyl-5-(2-(methylamino)-8,9-dihydroimidazo[1′,2′:1,6]pyrido[2,3-d]pyrimidin-6-yl)phenyl)-4-(trifluoromethyl)picolinamide

A mixture of 4-(trifluoromethyl)picolinic acid (1.01 g, 5.29 mmol) and HATU (2.81 g, 7.4 mmol) in DMF (75.0 mL) was stirred at r.t. for 0.5 h, then 6-(5-amino-4-fluoro-2-methylphenyl)-N-methyl-8,9-dihydroimidazo[1′,2′:1,6]pyrido[2,3-d]pyrimidin-2-amine (1.85 g, 5.716 mmol) was added, the mixture was stirred at r.t. for 1.5 h. The reaction mixture was added into water (1.0 L), stirred at r.t. for 0.5 h, filtered. The collected filtered cake was purified by column chromatography (DCM/MeOH=15/1, +0.1% NH₃-MeOH) to afford N-(2-fluoro-4-methyl-5-(2-(methylamino)-8,9-dihydroimidazo[1′,2′:1,6]pyrido[2,3-d]pyrimidin-6-yl)phenyl)-4-(trifluoromethyl)picolinamide (1.709 g, 65% yield) as a yellow solid. ¹H NMR (400 MHz, DMSO-d₆): δ 10.46 (s, 1H), 9.05 (d, J=5.2 Hz, 1H), 8.33 (s, 1H), 8.24-8.21 (m, 1H), 8.13-8.12 (m, 1H), 7.82 (d, J=8.0 Hz, 1H), 7.45-7.43 (m, 1H), 7.25 (d, J=12.0 Hz, 1H), 7.16 (s, 1H), 4.07-3.98 (m, 2H), 3.96-3.88 (m, 2H), 2.84 (d, J=3.2 Hz, 3H), 2.23 (s, 3H). LCMS (M+H⁺) m/z: 498.2.

### Example 140: Preparation of N-(2-fluoro-4-methyl-3-(2-(methylamino)-8,9-dihydroimidazo[1′,2′:1,6]pyrido[2,3-d]pyrimidin-6-yl)phenyl)-4-(trifluoromethyl)picolinamide (Compound 140)

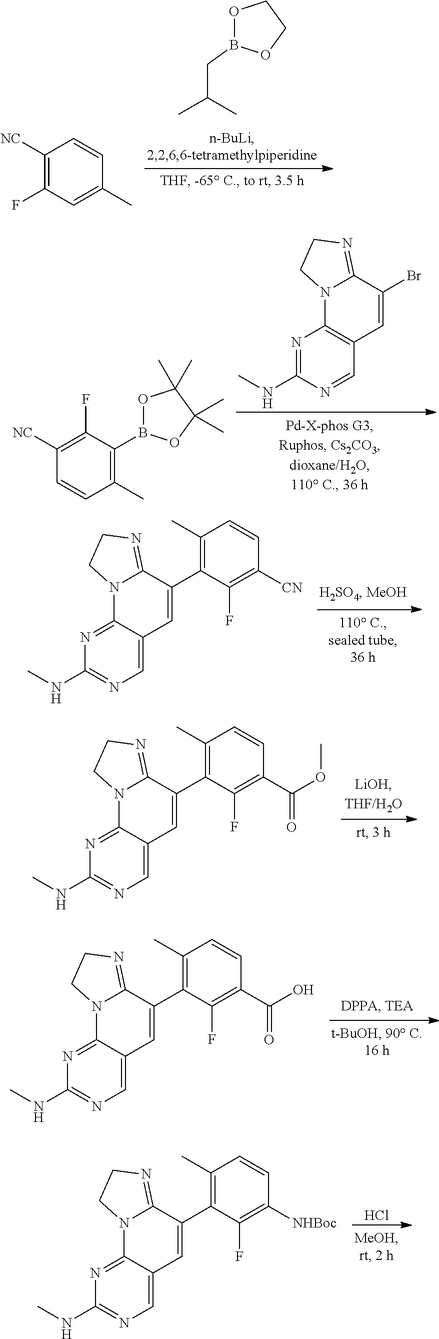

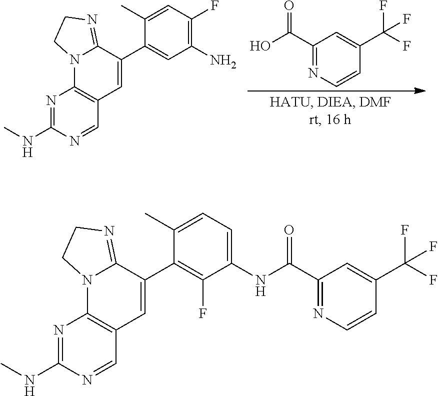 [SMILES: CNc1ncc2c(n1)N1CCN=C1C(c1cc(N)c(F)cc1C)=C2 ; O=C(O)c1cc(C(F)(F)F)ccn1 ; CNc1ncc2c(n1)N1CCN=C1C(c1c(C)ccc(NC(=O)c3cc(C(F)(F)F)ccn3)c1F)=C2]

### Step 1: Synthesis of 2-fluoro-4-methyl-3-(4,4,5,5-tetramethyl-1,3,2-dioxaborolan-2-yl)benzonitrile

To a solution of 2,2,6,6-tetramethylpiperidine (8.3 g, 59.2 mmol) in dry THF (20 mL) was added n-BuLi (22 mL, 55.5 mmol) at −65° C. under N₂. After being stirring at −65° C. for 1 h, 2-fluoro-4-methylbenzonitrile (5.0 g, 37 mmol) in dry THE (10 mL) was slowly added. The mixture was stirred at −65° C. under N₂ for 1 h. 2-Isopropoxy-4,4,5,5-tetramethyl-1,3,2-dioxaborolane (9.6 g, 51.8 mmol) was added into the mixture. The reaction mixture was stirred at −65° C. for 30 min, then warmed to rt for 1 h. Concentration in vacuum and purification on silica gel column chromatography (PE/EA=20:1) gave 2-fluoro-4-methyl-3-(4,4,5,5-tetramethyl-1,3,2-dioxaborolan-2-yl)benzonitrile (3.84 g, 48.1% yield) as a yellow solid.

### Step 2: Synthesis of 2-fluoro-4-methyl-3-(2-(methylamino)-8,9-dihydroimidazo[1′,2′:1,6]pyrido[2,3-d]pyrimidin-6-yl)benzonitrile

To a solution of 2-fluoro-4-methyl-3-(4,4,5,5-tetramethyl-1,3,2-dioxaborolan-2-yl)benzonitrile (1.0 g, 3.83 mmol) in dioxane/H₂O (20 mL/4 mL) was added 6-bromo-N-methyl-8,9-dihydroimidazo[1′,2′:1,6]pyrido[2,3-d]pyrimidin-2-amine (962 mg, 3.45 mmol), Cs2CO3 (3.7 g, 11.49 mmol), Ruphos (179 mg, 0.383 mmol) and Pd-X-phos G3 (324 mg, 0.383 mmol). The reaction mixture was stirred at 110° C. under N2 for 36 h. Concentration and purification by on silica gel column chromatography (DCM/MeOH=10:1) gave 2-fluoro-4-methyl-3-(2-(methylamino)-8,9-dihydroimidazo[1′,2′:1,6]pyrido[2,3-d]pyrimidin-6-yl)benzonitrile (1.0 g, 78.2% yield) as a yellow solid.

### Step 3: Synthesis of methyl 2-fluoro-4-methyl-3-(2-(methylamino)-8,9-dihydroimidazo[1′,2′:1,6]pyrido[2,3-d]pyrimidin-6-yl)benzoate

To a solution of 2-fluoro-4-methyl-3-(2-(methylamino)-8,9-dihydroimidazo[1′,2′:1,6]pyrido[2,3-d]pyrimidin-6-yl)benzonitrile (1.0 g, 2.994 mmol) in MeOH (15 mL) was added H2SO4 (3 mL), the reaction mixture was stirred at 110° C. in sealed tube for 36 h. Concentration and purification on flash (0.1% NH3H2O) afforded methyl 2-fluoro-4-methyl-3-(2-(methylamino)-8,9-dihydroimidazo[1′,2′:1,6]pyrido[2,3-d]pyrimidin-6-yl)benzoate (600 mg, 54.6% yield) as a yellow solid.

### Step 4: Synthesis of 2-fluoro-4-methyl-3-(2-(methylamino)-8,9-dihydroimidazo[1′,2′:1,6]pyrido[2,3-d]pyrimidin-6-yl)benzoic acid

To a solution of methyl 2-fluoro-4-methyl-3-(2-(methylamino)-8,9-dihydroimidazo[1′,2′:1,6]pyrido[2,3-d]pyrimidin-6-yl)benzoate (600 mg, 1.635 mmol) in MeOH/H2O (5 mL/5 mL) was added LiOH (137 mg, 3.27 mmol). The reaction mixture was stirred at rt for 3 h, Concentration and purification on flash (0.1% NH₃H₂O) gave 2-fluoro-4-methyl-3-(2-(methylamino)-8,9-dihydroimidazo[1′,2′:1,6]pyrido[2,3-d]pyrimidin-6-yl)benzoic acid (350 mg, 60.6%) as a yellow solid.

### Step 5: Synthesis of tert-butyl (2-fluoro-4-methyl-3-(2-(methylamino)-8,9-dihydroimidazo[1′,2′:1,6]pyrido[2,3-d]pyrimidin-6-yl)phenyl)carbamate

To a solution of 2-fluoro-4-methyl-3-(2-(methylamino)-8,9-dihydroimidazo[1′,2′:1,6]pyrido[2,3-d]pyrimidin-6-yl)benzoic acid (350 mg, 0.99 mmol) in t-BuOH (20 mL) was added DPPA (409 mg, 1.49 mmol) and TEA (300 mg, 2.98 mmol). The reaction mixture was stirred at 90° C. for 16 h. Concentration and purification on flash (0.1% NH3H2O) afforded tert-butyl (2-fluoro-4-methyl-3-(2-(methylamino)-8,9-dihydroimidazo[1′,2′:1,6]pyrido[2,3-d]pyrimidin-6-yl)phenyl)carbamate (321 mg, 76.4% yield) as a yellow solid.

### Step 6: Synthesis of 6-(3-amino-2-fluoro-6-methylphenyl)-N-methyl-8,9-dihydroimidazo[1′,2′:1,6]pyrido[2,3-d]pyrimidin-2-amine

To a solution of tert-butyl (2-fluoro-4-methyl-3-(2-(methylamino)-8,9-dihydroimidazo[1′,2′:1,6]pyrido[2,3-d]pyrimidin-6-yl)phenyl)carbamate (321 mg, 0.757 mmol) in MeOH (2 mL) was added HCl (1 mL). The reaction mixture was stirred at rt for 16 h. Concentration and purification on flash (0.1% NH₃H₂O) afforded 6-(3-amino-2-fluoro-6-methylphenyl)-N-methyl-8,9-dihydroimidazo[1′,2′:1,6]pyrido[2,3-d]pyrimidin-2-amine (150 mg, 46.6% yield) as a yellow solid.

### Step 7: Synthesis of N-(2-fluoro-4-methyl-3-(2-(methylamino)-8,9-dihydroimidazo[1′,2′:1,6]pyrido[2,3-d]pyrimidin-6-yl)phenyl)-4-(trifluoromethyl)picolinamide

To a mixture of 6-(3-amino-2-fluoro-6-methylphenyl)-N-methyl-8,9-dihydroimidazo[1′,2′:1,6]pyrido[2,3-d]pyrimidin-2-amine (40 mg, 0.12 mmol, 1.0 eq) and 4-(trifluoromethyl)picolinic acid (28 mg, 0.30 mmol, 1.2 eq) in DMF (3 mL) was added DIEA (0.1 mL) and HATU (70 mg, 0.15 mmol, 1.5 eq). The mixture was stirred at 20° C. overnight. LCMS showed the reaction was OK. Water was added, the reaction mixture was extracted with EA. The combined extracts were washed with brine. Concentration and purification by prep-HPLC (0.1% NH₄CO₃) gave N-(2-fluoro-4-methyl-3-(2-(methylamino)-8,9-dihydroimidazo[1′,2′:1,6]pyrido[2,3-d]pyrimidin-6-yl)phenyl)-4-(trifluoromethyl)picolinamide (21.0 mg, 35.2%) as a white solid. ¹H NMR (400 MHz, DMSO-d₆): δ 9.04 (d, J=5.2 Hz, 1H), 8.35 (s, 1H), 8.13-8.12 (m, 2H), 7.93-7.89 (m, 2H), 7.47 (s, 1H), 7.19-7.15 (m, 2H), 4.06-3.86 (m, 4H), 3.17 (s, 3H), 2.21 (s, 3H). LCMS (M+H⁺) m/z: 498.1.

### Example 141: Preparation of N-(2-fluoro-4-methyl-3-(2-(methylamino)-8,9-dihydroimidazo[1′,2′:1,6]pyrido[2,3-d]pyrimidin-6-yl)phenyl)benzamide (Compound 141)

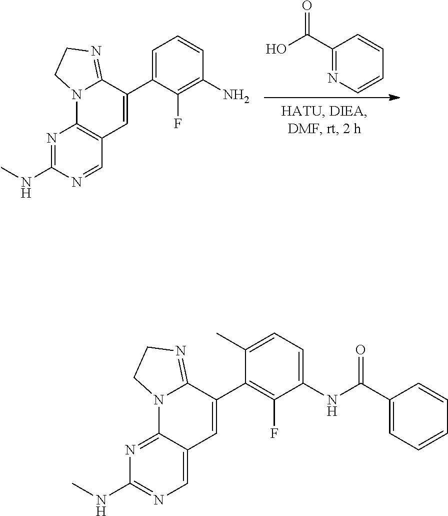 [SMILES: CNc1ncc2c(n1)N1CCN=C1C(c1cccc(N)c1F)=C2 ; O=C(O)c1ccccn1 ; CNc1ncc2c(n1)N1CCN=C1C(c1c(C)ccc(NC(=O)c3ccccc3)c1F)=C2]

### Step 1: Synthesis of N-(2-fluoro-4-methyl-3-(2-(methylamino)-8,9-dihydroimidazo[1′,2′:1,6]pyrido[2,3-d]pyrimidin-6-yl)phenyl)benzamide

A mixture of benzoic acid (17 mg, 0.139 mmol) and HATU (70 mg, 0.184 mmol) in DMF (3 mL) was stirred at rt for 15 mins, then the 6-(3-amino-2-fluoro-6-methylphenyl)-N-methyl-8,9-dihydroimidazo[1′,2′:1,6]pyrido[2,3-d]pyrimidin-2-amine (30 mg, 0.092 mmol) and DIEA (36 mg, 0.276 mmol) was added. The reaction was stirred at rt for 16 h. The resulting mixture was purified by prep-HPLC (0.1% HCl) to afford N-(2-fluoro-4-methyl-3-(2-(methylamino)-8,9-dihydroimidazo[1′,2′:1,6]pyrido[2,3-d]pyrimidin-6-yl)phenyl)benzamide (8.8 mg, 22.3% yield) as a yellow solid. ¹H NMR (400 MHz, DMSO-d₆): δ 10.17 (s, 1H), 9.96 (s, 1H), 8.88 (s, 1H), 8.59-8.56 (m, 1H), 8.20 (s, 1H), 7.98-7.96 (m, 2H), 7.70 (t, J=8.0 Hz, 1H), 7.60 (t, J=7.2 Hz, 1H), 7.55-7.52 (m, 2H), 7.26 (d, J=8.0 Hz, 1H), 4.69-4.64 (m, 2H), 4.04 (t, J=10.0 Hz, 2H), 2.97 (d, J=4.8 Hz, 3H), 2.21 (s, 3H). LCMS (M+H⁺) m/z: 429.5.

### Example 142: Preparation of N-(2-fluoro-4-methyl-3-(2-(methylamino)-8,9-dihydroimidazo[1′,2′:1,6]pyrido[2,3-d]pyrimidin-6-yl)phenyl)picolinamide (Compound 142)

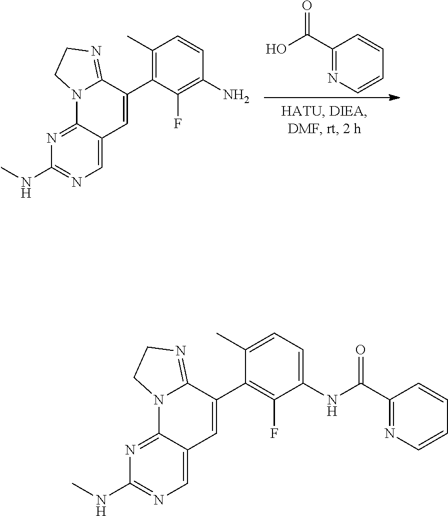 [SMILES: CNc1ncc2c(n1)N1CCN=C1C(c1c(C)ccc(N)c1F)=C2 ; O=C(O)c1ccccn1 ; CNc1ncc2c(n1)N1CCN=C1C(c1c(C)ccc(NC(=O)c3ccccn3)c1F)=C2]

### Step 1: Synthesis of N-(2-fluoro-4-methyl-3-(2-(methylamino)-8,9-dihydroimidazo[1′,2′:1,6]pyrido[2,3-d]pyrimidin-6-yl)phenyl)picolinamide

A mixture of picolinic acid (17 mg, 0.139 mmol) and HATU (70 mg, 0.184 mmol) in DMF (3 mL) was stirred at rt for 15 mins, then the 6-(3-amino-2-fluoro-6-methylphenyl)-N-methyl-8,9-dihydroimidazo[1′,2′:1,6]pyrido[2,3-d]pyrimidin-2-amine (30 mg, 0.092 mmol) and DIEA (36 mg, 0.276 mmol) was added. The reaction was stirred at rt for 16 h. The resulting mixture was purified by prep-HPLC (0.1% FA) to afford N-(2-fluoro-4-methyl-3-(2-(methylamino)-8,9-dihydroimidazo[1′,2′:1,6]pyrido[2,3-d]pyrimidin-6-yl)phenyl)picolinamide (18.2 mg, 45.8% yield) as a yellow solid. ¹H NMR (400 MHz, DMSO-d₆): δ 10.32 (s, 1H), 8.73 (d, J=4.0 Hz, 1H), 8.25-8.16 (m, 3H), 8.10-8.04 (m, 2H), 7.70 (d, J=8.4 Hz, 1H), 7.49 (br, 1H), 7.23 (s, 1H), 7.15 (d, J=8.8 Hz, 1H), 4.09-4.00 (m, 2H), 3.92-3.88 (m, 2H), 2.85 (s, 3H), 2.20 (s, 3H). LCMS (M+H⁺) m/z: 430.1.

### Example 143: Preparation of N-(2-fluoro-4-methyl-3-(2-(methylamino)-8,9-dihydroimidazo[1′,2′:1,6]pyrido[2,3-d]pyrimidin-6-yl)phenyl)but-2-ynamide (Compound 143)

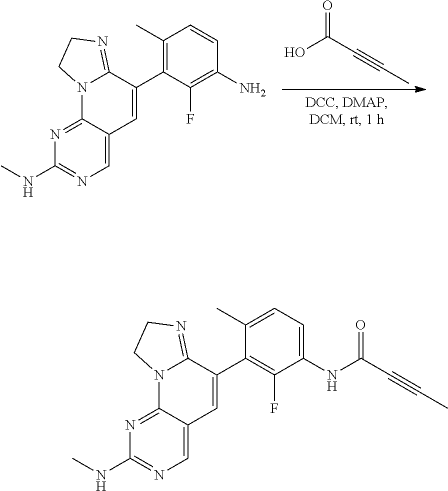

### Step 1: Synthesis of N-(2-fluoro-4-methyl-3-(2-(methylamino)-8,9-dihydroimidazo[1′,2′:1,6]pyrido[2,3-d]pyrimidin-6-yl)phenyl)but-2-ynamide

To a solution of 6-(3-amino-2-fluoro-6-methylphenyl)-N-methyl-8,9-dihydroimidazo[1′,2′:1,6]pyrido[2,3-d]pyrimidin-2-amine (30 mg 0.092 mmol) in DCM (3 mL) was added but-2-ynoic acid (12 mg, 0.138 mmol), DMAP (22 mg, 0.184 mmol) and DCC (28 mg, 0.138 mmol) in DCM (3 mL) at 0° C. The reaction mixture was stirred at rt for 1 h. The resulting mixture was purified by prep-HPLC (0.1% FA) to afford N-(2-fluoro-4-methyl-3-(2-(methylamino)-8,9-dihydroimidazo[1′,2′:1,6]pyrido[2,3-d]pyrimidin-6-yl)phenyl)but-2-ynamide (13.8 mg, 38.4% yield) as a yellow solid. ¹H NMR (400 MHz, DMSO-d₆): δ 10.27 (s, 1H), 8.22-8.19 (m, 2H), 7.49-7.46 (m, 2H), 7.15 (s, 1H), 7.04 (d, J=8.0 Hz, 1H), 4.04-3.96 (m, 2H), 3.89 (d, J=9.2 Hz, 2H), 2.84 (d, J=4.0 Hz, 3H), 2.15 (s, 3H), 2.03 (s, 3H). LCMS (M+H⁺) m/z: 391.1.

### Example 144: Preparation of N-(4-chloro-2-fluoro-5-(2-(methylamino)-8,9-dihydroimidazo[1′,2′:1,6]pyrido[2,3-d]pyrimidin-6-yl)phenyl)-4-(trifluoromethyl)picolinamide (Compound 144)

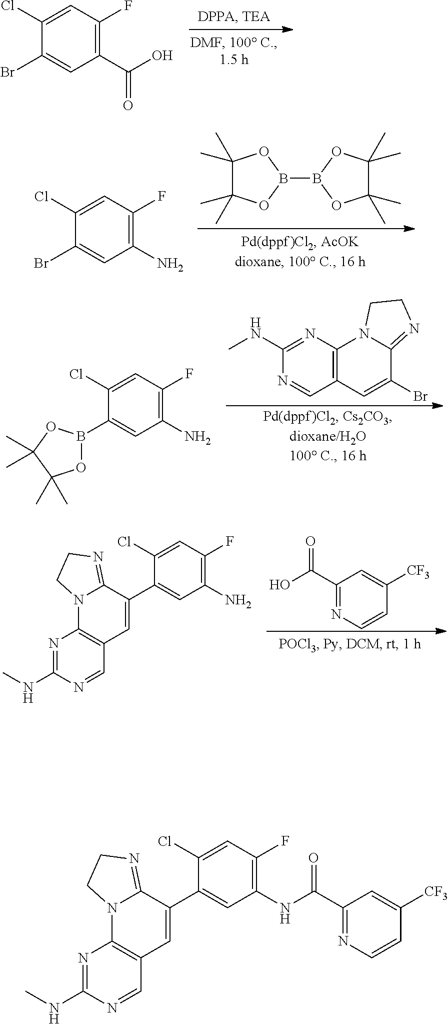

### Step 1: Synthesis of 5-bromo-4-chloro-2-fluoroaniline

To a solution of 5-bromo-4-chloro-2-fluorobenzoic acid (1.5 g, 5.9 mmol) and TEA (1.8 g, 17.7 mmol) in DMF (30 mL) was added DPPA (2.44 g, 8.9 mmol) at 0° C. The solution was stirred at 0° C. under N2 for 3 hrs. Then the solution was stirred at 80° C. under N2 for 1.5 hrs. H2O (4.3 g, 236 mmol) was added, the solution was stirred at 100° C. under N2 for 16 hrs. The reaction solution was diluted with EA (100 mL), washed with water (30 mL*3). The organic phase was concentrated and purified by silica gel chromatography (PE/EA=10/1) to get crude product. The crude was re-purified by flash chromatography to afford 5-bromo-4-chloro-2-fluoroaniline (140 mg, 10% yield) as a yellow solid. LCMS (M+H⁺) m/z: 225.9.

### Step 2: Synthesis of 4-chloro-2-fluoro-5-(4,4,5,5-tetramethyl-1,3,2-dioxaborolan-2-yl)aniline

A mixture of 5-bromo-4-chloro-2-fluoroaniline (140 mg, 0.623 mmol), 4,4,4′,4′,5,5,5′,5′-octamethyl-2,2′-bi(1,3,2-dioxaborolane) (237 mg, 0.935 mmol), KOAc (183 mg, 1.869 mmol) and Pd(dppf)Cl2 (182 mg, 0.249 mmol) in dioxane (10 mL) was stirred at 100° C. under N2 for 16 hrs. The reaction mixture was cooled to r.t. and concentrated. The residue was purified by silica gel chromatography (PE to PE/EA=10/1) to afford 4-chloro-2-fluoro-5-(4,4,5,5-tetramethyl-1,3,2-dioxaborolan-2-yl)aniline (169 mg, 100% yield) as a light green solid. LCMS (M+H+) m/z: 272.1.

### Step 3: Synthesis of 6-(5-amino-2-chloro-4-fluorophenyl)-N-methyl-8,9-dihydroimidazo[1′,2′:1,6]pyrido[2,3-d]pyrimidin-2-amine

A mixture of 4-chloro-2-fluoro-5-(4,4,5,5-tetramethyl-1,3,2-dioxaborolan-2-yl)aniline (140 mg, 0.50 mmol), 6-bromo-N-methyl-8,9-dihydroimidazo[1′,2′:1,6]pyrido[2,3-d]pyrimidin-2-amine (136 mg, 0.50 mmol), Cs2CO3 (488 mg, 1.50 mmol) and Pd(dppf)Cl₂ (73 mg, 0.10 mmol) in dioxane (20 mL) and water (5 mL) was stirred at 100° C. under N2 for 2 hrs. The reaction mixture was cooled to r.t. and concentrated. The residue was purified by flash chromatography to afford 6-(5-amino-2-chloro-4-fluorophenyl)-N-methyl-8,9-dihydroimidazo[1′,2′:1,6]pyrido[2,3-d]pyrimidin-2-amine (90 mg, 52% yield) as a yellow solid. LCMS (M+H⁺) m/z: 345.2.

### Step 4: Synthesis of N-(4-chloro-2-fluoro-5-(2-(methylamino)-8,9-dihydroimidazo[1′,2′:1,6]pyrido[2,3-d]pyrimidin-6-yl)phenyl)-4-(trifluoromethyl)picolinamide hydrochloride

To a solution of 6-(5-amino-2-chloro-4-fluorophenyl)-N-methyl-8,9-dihydroimidazo[1′,2′:1,6]pyrido[2,3-d]pyrimidin-2-amine (40 mg, 0.116 mmol), 4-(trifluoromethyl)picolinic acid (22 mg, 0.116 mmol) and pyridine (3 drops) in DCM (6 mL) was added POCl3 (1 drop). The solution was stirred at rt for 15 mins. The reaction solution was diluted with DCM (30 mL), washed with water (10 mL) and concentrated. The residue was purified by prep-HPLC (0.1%/HCl/CH3CN/H2O) to afford N-(4-chloro-2-fluoro-5-(2-(methylamino)-8,9-dihydroimidazo[1′,2′:1,6]pyrido[2,3-d]pyrimidin-6-yl)phenyl)-4-(trifluoromethyl)picolinamide hydrochloride (19.5 mg, 30% yield) as a yellow solid. ¹H NMR (400 MHz, DMSO-d₆): δ 10.70 (s, 1H), 10.10 (s, 1H), 9.09 (d, J=5.2 Hz, 1H), 8.91 (s, 1H), 8.63-8.35 (m, 1H), 8.35 (s, 1H), 8.25 (s, 1H), 8.21-8.17 (m, 2H), 7.90 (d, J=10.4 Hz, 1H), 4.71-4.61 (m, 2H), 4.10-4.05 (m, 2H), 2.98 (d, J=4.8 Hz, 3H). LCMS (M+H⁺) m/z: 518.2.

### Example 145: Preparation of N-(4-methyl-3-(2-(methylamino)-8,9-dihydroimidazo[1′,2′:1,6]pyrido[2,3-d]pyrimidin-6-yl)phenyl)-2-(trifluoromethyl)isonicotinamide (Compound 145)

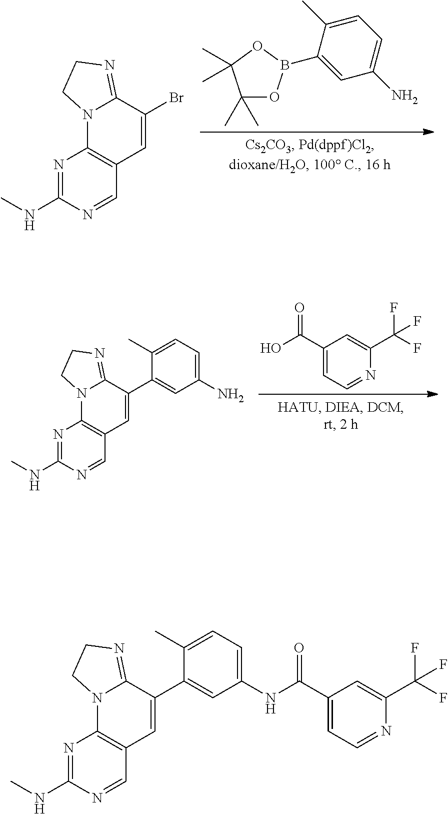

The preparation of 6-bromo-N-methyl-8,9-dihydroimidazo[1′,2′:1,6]pyrido[2,3-d]pyrimidin-2-amine was described in Example 133.

### Step 1: Synthesis of 6-(5-amino-2-methylphenyl)-N-methyl-8,9-dihydroimidazo[1′,2′:1,6]pyrido[2,3-d]pyrimidin-2-amine

A mixture of 6-bromo-N-methyl-8,9-dihydroimidazo[1′,2′:1,6]pyrido[2,3-d]pyrimidin-2-amine (390 mg, 1.39 mmol), 4-methyl-3-(4,4,5,5-tetramethyl-1,3,2-dioxaborolan-2-yl)aniline (389 mg, 1.67 mmol), Cs₂CO₃ (1.36 g, 4.18 mmol) and Pd(dppf)Cl₂ (102 mg, 0.14 mmol) in dioxane (20 mL) and water (2 mL) was degassed and charged with N₂ three times and stirred at 100° C. for 16 h. The reaction mixture was cooled to r.t., filtered and concentrated. The residue was purified column chromatography (DCM/MeOH=20/1, +0.5% TEA) to afford 6-(5-amino-2-methylphenyl)-N-methyl-8,9-dihydroimidazo[1′,2′:1,6]pyrido[2,3-d]pyrimidin-2-amine (240 mg, 56.3% yield) as a gray solid. LCMS (M+H⁺) m/z: 307.2.

### Step 2: Synthesis of N-(4-methyl-3-(2-(methylamino)-8,9-dihydroimidazo[1′,2′:1,6]pyrido[2,3-d]pyrimidin-6-yl)phenyl)-2-(trifluoromethyl)isonicotinamide

DIEA (50.4 mg, 0.39 mmol) was added to the mixture of 6-(5-amino-2-methylphenyl)-N-methyl-8,9-dihydroimidazo[1′,2′:1,6]pyrido[2,3-d]pyrimidin-2-amine (40 mg, 0.13 mmol), 2-(trifluoromethyl)isonicotinic acid (30 mg, 0.16 mmol), HATU (74.4 mg, 0.20 mmol) in DCM (3 mL). The mixture was stirred at r.t for 2 hours. The reaction mixture was diluted with H₂O (30 mL) and extracted with DCM (30 mL×3). The combined organic layers were washed with brine (50 mL) and dried over Na₂SO₄, filtered, concentrated to give the crude product, which was purified by Prep-HPLC (0.1% NH₃·H₂O) to afford N-(4-methyl-3-(2-(methylamino)-8,9-dihydroimidazo[1′,2′:1,6]pyrido[2,3-d]pyrimidin-6-yl)phenyl)-2-(trifluoromethyl)isonicotinamide (4.7 mg, 6.0% yield) as a yellow solid. ¹H NMR (400 MHz, DMSO-d₆): δ 10.64 (s, 1H), 8.98 (d, J=5.2 Hz, 1H), 8.37 (s, 1H), 8.26-8.18 (m, 2H), 7.67 (dd, J=8.4, 2.0 Hz, 1H), 7.63-7.62 (m, 1H), 7.41-7.39 (m, 1H), 7.25 (d, J=8.4 Hz, 1H), 7.12 (s, 1H), 4.01-4.00 (m, 2H), 3.93-3.91 (m, 2H), 2.84 (d, J=4.0 Hz, 3H) 2.21 (s, 3H). LCMS (M+H⁺) m/z: 480.2.

### Example 146: Preparation of N-(4-methyl-3-(2-(methylamino)-8,9-dihydroimidazo[1′,2′:1,6]pyrido[2,3-d]pyrimidin-6-yl)phenyl)-6-(trifluoromethyl)pyrazine-2-carboxamide (Compound 146)

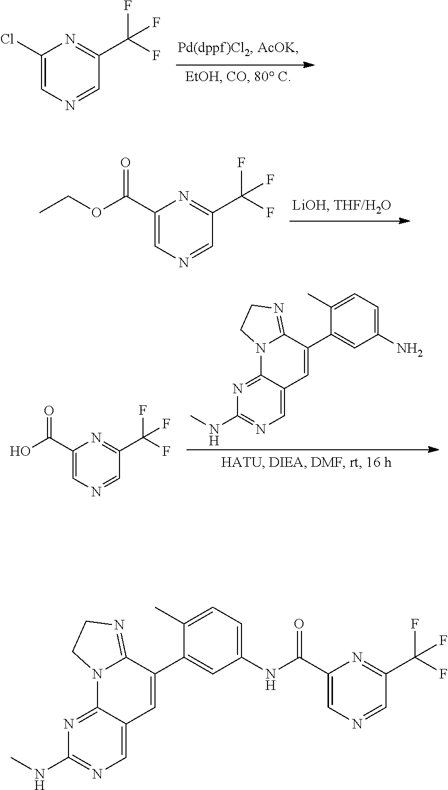

### Step 1: Synthesis of ethyl 6-(trifluoromethyl)pyrazine-2-carboxylate

A mixture of 2-chloro-6-(trifluoromethyl)pyrazine (400 mg, 2.2 mmol), AcOK (647 mg, 6.6 mmol) and [1,1′-Bis(diphenylphosphino)ferrocene]dichloropalladium(II) (161 mg, 0.22 mmol) in EtOH (15.0 mL). The resulting mixture was degassed and charged with N₂ three times, stirred at 80° C. for 4 h. The reaction mixture was concentrated and purified by silica column (PE:EA=5:1) to afford ethyl 6-(trifluoromethyl)pyrazine-2-carboxylate (560 mg, 93% yield) as yellow oil. LCMS (M+H⁺) m/z: 221.0.

### Step 2: Synthesis of 6-(trifluoromethyl)pyrazine-2-carboxylic acid

A mixture of ethyl 6-(trifluoromethyl)pyrazine-2-carboxylate (510 mg, 2.32 mmol) and LiOH—H₂O (584 mg, 13.9 mmol) in THF (10.0 mL) and H₂O (10.0 mL) was stirred at 25° C. for 2.5 h. Then 2N HCl was added into the reaction mixture to pH=5˜6, the reaction mixture was extracted with EA (50 mL×2). The combined organic phase was washed with brine, dried over Na₂SO₄, filtered and concentrated to afford 6-(trifluoromethyl)pyrazine-2-carboxylic acid (410 mg, 93% yield) as an off-white solid. LCMS (M−H)⁻ m/z: 191.1.

### Step 3: Synthesis of N-(4-methyl-3-(2-(methylamino)-8,9-dihydroimidazo[1′,2′:1,6]pyrido[2,3-d]pyrimidin-6-yl)phenyl)-6-(trifluoromethyl)pyrazine-2-carboxamide

To a mixture of 6-(trifluoromethyl)pyrazine-2-carboxylic acid (36 mg, 0.187 mmol), 6-(5-amino-2-methylphenyl)-N-methyl-8,9-dihydroimidazo[1′,2′:1,6]pyrido[2,3-d]pyrimidin-2-amine (60 mg, 0.196 mmol) and HATU (142 mg, 0.374 mmol) in DMF (3.0 mL) was added DIEA (48 mg, 0.374 mmol). The resulting mixture was stirred at r.t. for 16 h under N₂. The reaction mixture was added into water (40.0 mL) slowly, stirred at r.t. for 30 min, filtered. The collected cake was purified by silica column chromatography (DCM:MeOH=15:1, +0.1% NH₃-MeOH) to afford N-(4-methyl-3-(2-(methylamino)-8,9-dihydroimidazo[1′,2′:1,6]pyrido[2,3-d]pyrimidin-6-yl)phenyl)-6-(trifluoromethyl)pyrazine-2-carboxamide (45.9 mg, 51% yield) as a yellow solid. ¹H NMR (400 MHz, DMSO-d₆): δ 10.60 (s, 1H), 9.54 (s, 1H), 9.44 (s, 1H), 8.25-8.22 (m, 1H), 7.77 (d, J=7.6 Hz, 1H), 7.72 (s, 1H), 7.43-7.41 (m, 1H), 7.25 (d, J=8.0 Hz, 1H), 7.15 (s, 1H), 4.07-4.01 (m, 2H), 3.93-3.89 (m, 2H), 2.85 (s, 3H), 2.21 (s, 3H). LCMS (M+H⁺) m/z: 481.4.

### Example 147: Preparation of N-(4-methyl-3-(2-(methylamino)-8,9-dihydroimidazo[1′,2′:1,6]pyrido[2,3-d]pyrimidin-6-yl)phenyl)-2-(trifluoromethyl)pyrimidine-4-carboxamide (Compound 147)

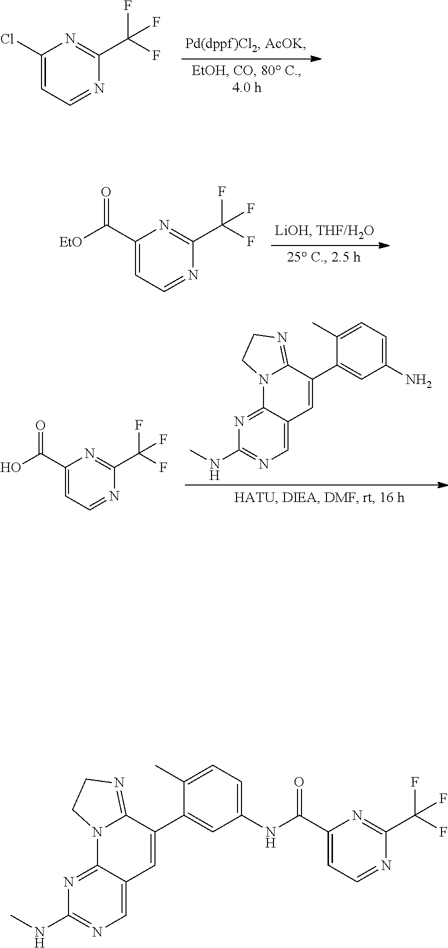

### Step 1: Synthesis of ethyl 2-(trifluoromethyl)pyrimidine-4-carboxylate

A mixture of 4-chloro-2-(trifluoromethyl)pyrimidine (150 mg, 0.55 mmol), AcOK (162 mg, 1.65 mmol) and [1,1′-bis(diphenylphosphino)ferrocene]dichloropalladium(II) (45 mg, 0.055 mmol) in EtOH (15.0 mL) was degassed and charged with N₂ three times, stirred at 80° C. for 4 h. The reaction mixture was concentrated and purified by silica column chromatography (PE:EA=5:1) to afford ethyl 2-(trifluoromethyl)pyrimidine-4-carboxylate (144 mg, 80% yield) as a yellow oil. LCMS (M+H⁺) m/z: 221.0.

### Step 2: Synthesis of 2-(trifluoromethyl)pyrimidine-4-carboxylic acid

To a solution of ethyl 2-(trifluoromethyl)pyrimidine-4-carboxylate (144 mg, 0.65 mmol) in THF (3.0 mL) and H₂O (3.0 mL) was added LiOH—H₂O (165 mg, 3.93 mmol). The resulting mixture was stirred at 25° C. for 2.5 h. Then 2N HCl was added into the reaction mixture to pH=5˜6, the reaction mixture was extracted with EA (50 mL×2). The combined organic phase was washed with brine, dried over Na₂SO₄, filtered and concentrated to afford 2-(trifluoromethyl)pyrimidine-4-carboxylic acid (98 mg, 78% yield) as an off-white solid. LCMS (M−H⁻) m/z: 191.1.

### Step 3: Synthesis of N-(4-methyl-3-(2-(methylamino)-8,9-dihydroimidazo[1′,2′:1,6]pyrido[2,3-d]pyrimidin-6-yl)phenyl)-2-(trifluoromethyl)pyrimidine-4-carboxamide

To a mixture of 2-(trifluoromethyl)pyrimidine-4-carboxylic acid (36 mg, 0.187 mmol), 6-(5-amino-2-methylphenyl)-N-methyl-8,9-dihydroimidazo[1′,2′:1,6]pyrido[2,3-d]pyrimidin-2-amine (60 mg, 0.196 mmol) and HATU (142 mg, 0.374 mmol) in DMF (3.0 mL) was added DIEA (48 mg, 0.374 mmol). The resulting mixture was stirred at r.t. for 16 h under N₂. The reaction mixture was added into water (40 mL) slowly, stirred at r.t. for 30 min, filtered. The collected cake was purified by silica column chromatography (DCM:MeOH=15:1, +0.1% NH₃-MeOH) to afford N-(4-methyl-3-(2-(methylamino)-8,9-dihydroimidazo[1′,2′:1,6]pyrido[2,3-d]pyrimidin-6-yl)phenyl)-2-(trifluoromethyl)pyrimidine-4-carboxamide (29.6 mg, 33% yield) as a yellow solid. ¹H NMR (400 MHz, DMSO-d₆): δ10.64 (s, 1H), 9.34 (d, J=4.8 Hz, 1H), 8.34 (d, J=4.8 Hz, 1H), 8.22-8.21 (m, 1H), 7.78-7.75 (m, 1H), 7.73 (s, 1H), 7.45-7.42 (m, 1H), 7.26 (d, J=8.0 Hz, 1H), 7.16 (s, 1H), 4.12-3.99 (m, 2H), 3.93-3.89 (m, 2H), 2.84 (s, 3H), 2.21 (s, 3H). LCMS (M+H⁺) m/z: 481.2.

### Example 148: Preparation of N-(4-methyl-3-(2-(methylamino)-8,9-dihydroimidazo[1′,2′:1,6]pyrido[2,3-d]pyrimidin-6-yl)phenyl)-4-(trifluoromethyl)pyrimidine-2-carboxamide (Compound 148)

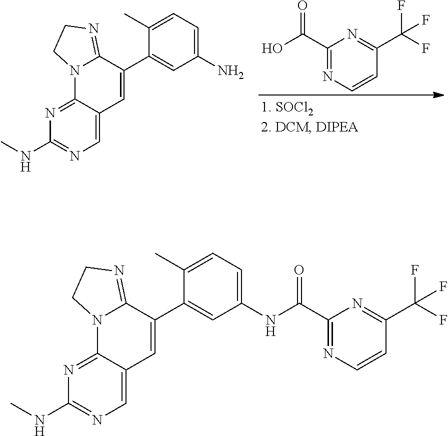 [SMILES: CNc1ncc2c(n1)N1CCN=C1C(c1cc(N)ccc1C)=C2 ; O=C(O)c1nccc(C(F)(F)F)n1 ; CNc1ncc2c(n1)N1CCN=C1C(c1cc(NC(=O)c3nccc(C(F)(F)F)n3)ccc1C)=C2]

### Step 1: Synthesis of N-(4-methyl-3-(2-(methylamino)-8,9-dihydroimidazo[1′,2′:1,6]pyrido[2,3-d]pyrimidin-6-yl)phenyl)-4-(trifluoromethyl)pyrimidine-2-carboxamide

The mixture of 4-(trifluoromethyl)pyrimidine-2-carboxylic acid (35 mg, 0.18 mmol) in SOCl2 (1.0 mL) was stirred at 80° C. for 2 h. The reaction mixture was concentrated and diluted with DCM (2.0 mL), then a solution of DIPEA (58 mg, 0.45 mmol) and 6-(5-amino-2-methylphenyl)-N-methyl-8,9-dihydroimidazo[1′,2′:1,6]pyrido[2,3-d]pyrimidin-2-amine (45 mg, 0.15 mmol) in DCM (4.0 mL). was added. The reaction mixture was stirred at 10° C. for 2 h. The reaction mixture was concentrated, purified by column chromatography (DCM:MeOH=10:1) and prep-HPLC (NH4HCO3) to give N-(4-methyl-3-(2-(methylamino)-8,9-dihydroimidazo[1′,2′:1,6]pyrido[2,3-d]pyrimidin-6-yl)phenyl)-4-(trifluoromethyl)pyrimidine-2-carboxamide (11.4 mg, 16% yield). ¹HNMR (400 MHz, DMSO-d₆): δ 10.83 (s, 1H), 9.39 (d, J=5.2 Hz, 1H), 8.35 (s, 1H), 8.27 (d, J=5.2 Hz, 1H), 7.77-7.75 (m, 2H), 7.37-7.27 (m, 3H), 4.20-4.05 (m, 2H), 3.96-3.91 (m, 2H), 2.87 (s, 3H), 2.21 (s, 3H). LCMS (M+H⁺) m/z: 481.0.

### Example 149: Preparation of N-(4-methyl-3-(2-(methylamino)-8,9-dihydroimidazo[1′,2′:1,6]pyrido[2,3-d]pyrimidin-6-yl)phenyl)-5-(trifluoromethyl)pyridazine-3-carboxamide (Compound 149)

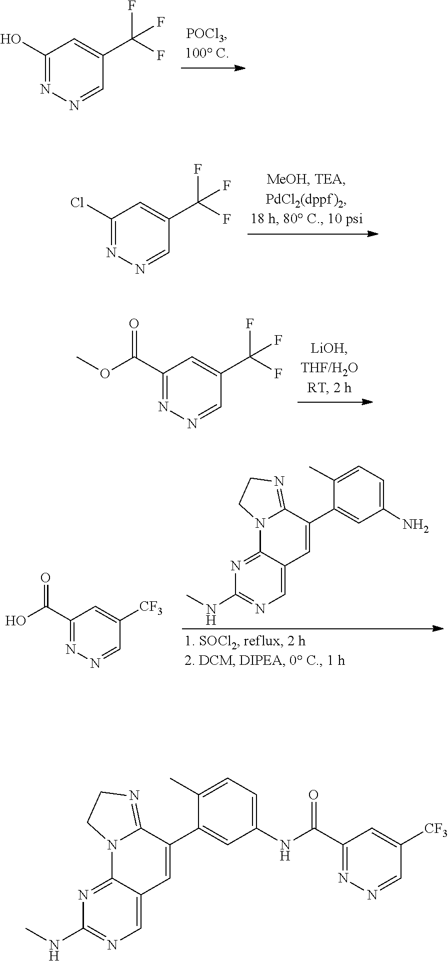

### Step 1: Synthesis of 3-chloro-5-(trifluoromethyl)pyridazine

A mixture of 5-(trifluoromethyl)pyridazin-3-ol (200 mg, 1.2 mmol) in POCl₃ (1.0 mL) was stirred at 110° C. for 2 h. The reaction mixture was poured into cold water, the pH was adjusted to 7 with 2N NaOH (aq). The reaction mixture was extracted with DCM (100 mL×5), the combined organic phase was purified by column chromatography (PE:EA=2:1) to give 3-chloro-5-(trifluoromethyl)pyridazine (100 mg, 48% yield) as solid. LCMS (M+H⁺) m/z: 184.0.

### Step 2: Synthesis of methyl 5-(trifluoromethyl)pyridazine-3-carboxylate

The mixture of 3-chloro-5-(trifluoromethyl)pyridazine (182 mg, 1.0 mmol), DIPEA (400 mg, 3.0 mmol), Pd(dppf)Cl₂ (73 mg, 0.1 mmol) in MeOH (15 mL) was stirred at 80° C. for 24 h under CO balloon The mixture was purified by column chromatography (PE:EA=10:1) to afford methyl 5-(trifluoromethyl)pyridazine-3-carboxylate (35 mg) as a white solid. LCMS (M+H⁺) m/z: 207.1.

### Step 3: Synthesis of 5-(trifluoromethyl)pyridazine-3-carboxylic acid

The mixture of methyl 5-(trifluoromethyl)pyridazine-3-carboxylate (35 mg, 0.15 mmol) and LiOH·H₂O (18 mg, 0.45 mmol) in THF (2.5 mL) and water (2.5 mL) was stirred for 2 h at 20° C. The pH was adjusted to 5 by 3 M HCl, the reaction mixture was extracted with EA (20 mL×2). The combined organic layers were washed with brine (20 mL), dried over Na₂SO₄, filtered and concentrated to give product 5-(trifluoromethyl)pyridazine-3-carboxylic acid (30 mg, 100% yield). LCMS (M+H⁺) m/z: 193.0.

### Step 4: Synthesis of N-(4-methyl-3-(2-(methylamino)-8,9-dihydroimidazo[1′,2′:1,6]pyrido[2,3-d]pyrimidin-6-yl)phenyl)-5-(trifluoromethyl)pyridazine-3-carboxamide

The mixture of 5-(trifluoromethyl)pyridazine-3-carboxylic acid (30 mg, 0.15 mmol) in SOCl₂ (1.0 mL) was stirred at 80° C. for 2 h. The reaction mixture was concentrated and diluted with DCM (2.0 mL), then a solution of DIPEA (47 mg, 0.36 mmol), 6-(5-amino-2-methylphenyl)-N-methyl-8,9-dihydroimidazo[1′,2′:1,6]pyrido[2,3-d]pyrimidin-2-amine (38 mg, 0.12 mmol) in DCM (4.0 mL) was added. The reaction mixture was stirred at 10° C. for 2 h. The reaction mixture was concentrated, purified by prep-HPLC (NH4HCO3) to give N-(4-methyl-3-(2-(methylamino)-8,9-dihydroimidazo[1′,2′:1,6]pyrido[2,3-d]pyrimidin-6-yl)phenyl)-5-(trifluoromethyl)pyridazine-3-carboxamide (23 mg, 40% yield) as yellow solid. ¹H NMR (400 MHz, DMSO-d6): 11.24 (s, 1H), 9.96 (d, J=1.6 Hz, 1H), 8.56 (d, J=1.2 Hz, 1H), 8.29 (s, 1H), 7.86 (s, 1H), 7.81 (d, J=8.0 Hz, 1H), 7.49 (br, 1H), 7.28-7.25 (m, 2H), 4.14-4.01 (m, 2H), 3.96-3.90 (m, 2H), 2.86 (s, 3H), 2.19 (s, 3H). LCMS (M+H⁺) m/z: 481.0.

### Example 150: Preparation of N-(4-methyl-3-(2-(methylamino)-8,9-dihydroimidazo[1′,2′:1,6]pyrido[2,3-d]pyrimidin-6-yl)phenyl)-6-(trifluoromethyl)pyridazine-4-carboxamide (Compound 150)

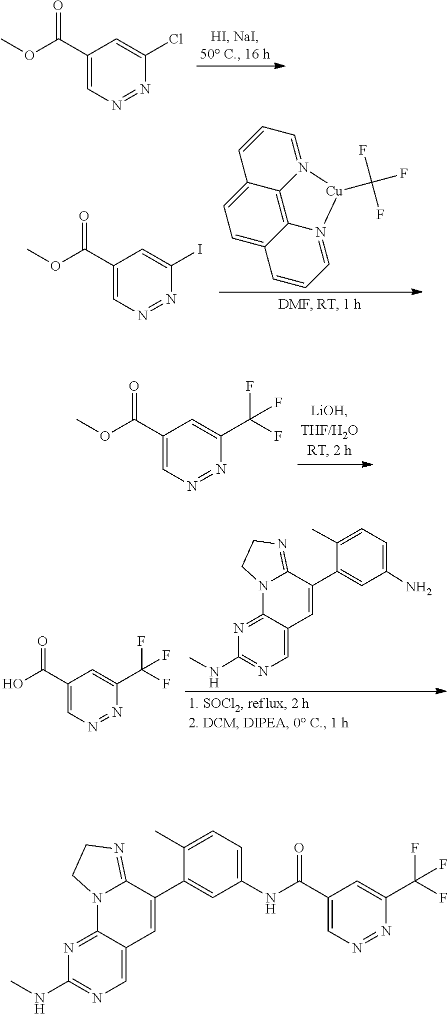

### Step 1: Synthesis of methyl 6-iodopyridazine-4-carboxylate

The solution of methyl 6-chloropyridazine-4-carboxylate (250 mg, 1.44 mmol) and NaI (314 mg, 2.10 mmol) in HI (2.0 mL) was stirred at 50° C. for 16 h. The reaction mixture was cooled down to room temperature and diluted with water (15 mL). The mixture was basified to pH=7 with saturated sodium bicarbonate aqueous solution and extracted with DCM (20 mL×2). The combined organic layers were washed with brine (20 mL), dried over Na₂SO₄, filtered and concentrated. The residue was purified by flash (PE:EA=5:1) to give methyl 6-iodopyridazine-4-carboxylate (188 mg, purity: 52%) as white solid. LCMS (M+H⁺) m/z: 265.0.

### Step 2: Synthesis of 6-(trifluoromethyl)pyridazine-4-carboxylate

The mixture of methyl 6-iodopyridazine-4-carboxylate (188 mg, 0.70 mmol) and (1,10-phenanthroline)(trifluoromethyl)copper (I) (218 mg, 0.70 mmol) in DMF (5 mL) was stirred at 20° C. for 1 h under dark. The reaction mixture was quenched with water (20 mL) and extracted with EA (10 mL×2). The combined organic layers were washed with brine (20 mL), dried over Na2SO4, filtered and concentrated. The residue was purification by flash (PE:EA=5:1) to give 6-(trifluoromethyl)pyridazine-4-carboxylate (50 mg purity: 35%) as an off-white solid. LCMS (M+H+) m/z: 207.1.

### Step 3: Synthesis of 6-(trifluoromethyl)pyridazine-4-carboxylic acid

The mixture of methyl 6-(trifluoromethyl)pyridazine-4-carboxylate (100 mg, 0.5 mmol) and LiOH·H₂O (60 mg, 1.5 mmol) were in THF (2.5 mL) and water (2.5 mL) was stirred at 20° C. for 2 h. The mixture was acidified to pH=5 with 3 M HCl, extracted with EA (20 mL×2). The combined organic layers were washed with brine (20 mL), dried over Na₂SO₄, filtered and concentrated to give 6-(trifluoromethyl)pyridazine-4-carboxylic acid (92 mg, 100% yield). LCMS (M+H⁺) m/z: 193.0.

### Step 4: Synthesis of N-(4-methyl-3-(2-(methylamino)-8,9-dihydroimidazo[1′,2′:1,6]pyrido[2,3-d]pyrimidin-6-yl)phenyl)-6-(trifluoromethyl)pyridazine-4-carboxamide

The mixture of 6-(trifluoromethyl)pyridazine-4-carboxylic acid (46 mg, 0.24 mmol) in SOCl₂ (1.0 mL) was stirred at 80° C. for 2 h. The reaction mixture was concentrated and diluted with DCM (2.0 mL), then a solution of DIPEA (78 mg, 0.60 mmol), 6-(5-amino-2-methylphenyl)-N-methyl-8,9-dihydroimidazo[1′,2′:1,6]pyrido[2,3-d]pyrimidin-2-amine (60 mg, 0.20 mmol) in DCM (4.0 mL) was added. The reaction mixture was stirred at 10° C. for 2 h. The reaction was concentrated and purified by Flash (DCM:MeOH=10:1) and prep-HPLC (NH₄HCO₃) to give N-(4-methyl-3-(2-(methylamino)-8,9-dihydroimidazo[1′,2′:1,6]pyrido[2,3-d]pyrimidin-6-yl)phenyl)-6-(trifluoromethyl)pyridazine-4-carboxamide (23.2 mg, 24% yield). ¹H NMR (400 MHz, DMSO-d6): δ 10.84 (s, 1H), 9.92 (d, J=2.0 Hz, 1H), 8.70 (d, J=2.0 Hz, 1H), 8.26 (br, 1H), 7.69-7.64 (m, 3H), 7.29-7.27 (m, 2H), 4.10-4.00 (m, 2H), 3.96-3.89 (m, 2H), 2.85 (s, 3H), 2.19 (s, 3H). LCMS (M+H⁺) m/z: 481.0.

### Example 151: Preparation of N-(4-methyl-3-(2-(methylamino)-8,9-dihydroimidazo[1′,2′:1,6]pyrido[2,3-d]pyrimidin-6-yl)phenyl)-6-(trifluoromethyl)pyrimidine-4-carboxamide (Compound 151)

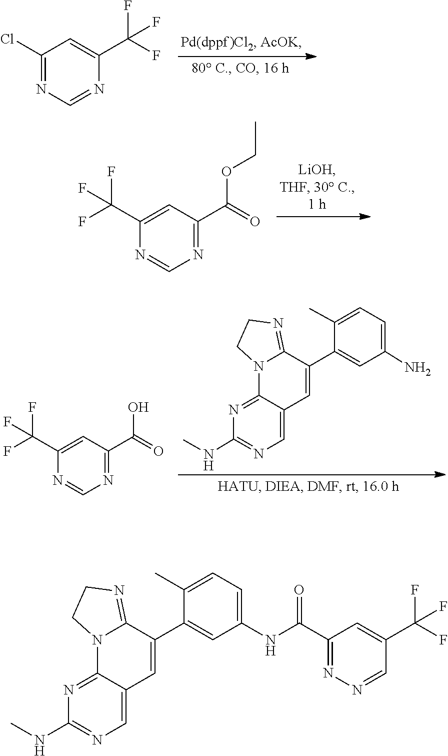

### Step 1: Synthesis of ethyl 6-(trifluoromethyl)pyrimidine-4-carboxylate

A mixture of 4-chloro-6-(trifluoromethyl)pyrimidine (1 g, 5.5 mmol), AcOK (2.156 g, 22 mmol), and Pd(dppf)Cl₂ (200 mg, 0.27 mmol) in EtOH (30 mL) was degassed, charged with CO three times and stirred at 80° C. for 16 h under CO. The reaction mixture was concentrated and purified by silica column chromatography (PE:EA=4:1) to afford ethyl 6-(trifluoromethyl)pyrimidine-4-carboxylate (230 mg, 19% yield) as a brown solid. LCMS (M+H⁺) m/z: 221.2.

### Step 2: Synthesis of 6-(trifluoromethyl)pyrimidine-4-carboxylic acid

A mixture of ethyl 6-(trifluoromethyl)pyrimidine-4-carboxylate (100 mg, 0.45 mmol), LiOH (76 mg, 2.72 mmol) in THF (5 mL) and H₂O (0.5 mL) was stirred at rt for 1 h. The reaction mixture was diluted with H₂O (20 mL) and pH was adjusted to 4-5 with HCl (2M). The reaction mixture was extracted with EA (20 mL×2). The organic layer was dried with NaSO₄, filtered and concentrated to give 6-(trifluoromethyl)pyrimidine-4-carboxylic acid (70 mg, crude) as a brown solid. LCMS (M+H⁺) m/z: 193.0.

### Step 3: Synthesis of N-(4-methyl-3-(2-(methylamino)-8,9-dihydroimidazo[1′,2′:1,6]pyrido[2,3-d]pyrimidin-6-yl)phenyl)-6-(trifluoromethyl)pyrimidine-4-carboxamide

To a solution of 6-(trifluoromethyl)pyrimidine-4-carboxylic acid (60 mg, crude), 6-(5-amino-2-methylphenyl)-N-methyl-8,9-dihydroimidazo[1′,2′:1,6]pyrido[2,3-d]pyrimidin-2-amine (100 mg, 0.33 mmol), and HATU (248 mg, 0.65 mmol) in DMF (3.0 mL) was added DIEA (84 mg, 0.65 mmol). The resulting mixture was stirred at r.t. for 16 h under N₂. The reaction mixture was added into water (40 mL) slowly, stirred at r.t. for 30 min, filtered. The collected cake was purified by silica column chromatography (DCM:MeOH=15:1, +0.1% NH₃-MeOH) to afford N-(4-methyl-3-(2-(methylamino)-8,9-dihydroimidazo[1′,2′:1,6]pyrido[2,3-d]pyrimidin-6-yl)phenyl)-6-(trifluoromethyl)pyrimidine-4-carboxamide (32.8 mg, 10.5% yield) as a yellow solid. ¹H NMR (400 MHz, DMSO-d₆): δ 10.97 (s, 1H), 9.68 (s, 1H), 8.45 (s, 1H), 8.26-8.24 (m, 1H), 7.82 (s, 1H), 7.78 (d, J=8.4 Hz, 1H), 7.57-7.45 (m, 1H), 7.19 (d, J=8.4 Hz, 1H), 7.17-7.16 (m, 1H), 4.11-4.02 (m, 2H), 3.98 (t, J=8.8 Hz, 2H), 2.86 (s, 3H), 2.22 (s, 3H). LCMS (M+H⁺) m/z: 481.7.

### Example 152: Preparation of 3-chloro-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (Compound 152)

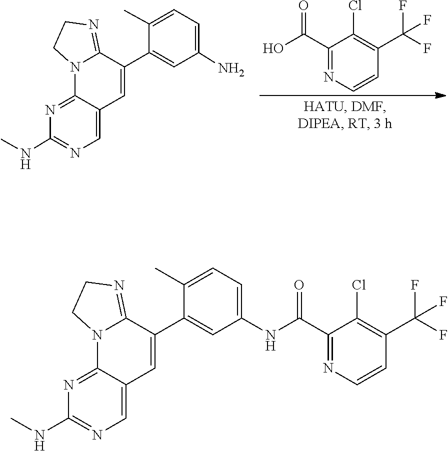 [SMILES: CNc1ncc2c(n1)N1CCN=C1C(c1cc(N)ccc1C)=C2 ; O=C(O)c1nccc(C(F)(F)F)c1Cl ; CNc1ncc2c(n1)N1CCN=C1C(c1cc(NC(=O)c3nccc(C(F)(F)F)c3Cl)ccc1C)=C2]

### Step 1: Synthesis of 3-chloro-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide

The mixture of 3-chloro-4-(trifluoromethyl)picolinic acid (40 mg, 0.18 mmol), HATU (68 mg, 0.18 mmol), DIPEA (58 mg, 0.45 mmol) and 6-(5-amino-2-methylphenyl)-N-methyl-8,9-dihydroimidazo[1′,2′:1,6]pyrido[2,3-d]pyrimidin-2-amine (46 mg, 0.15 mmol) in DMF (2.0 mL). The reaction mixture was stirred at 10° C. for 2 h, worked complete detected by LCMS. The reaction was purification by flash (DCM:MeOH=10:1) and then prep-HPLC (0.5% FA) to give 3-chloro-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (5.3 mg) as yellow solid. ¹H NMR (400 MHz, DMSO-d6): δ 10.80 (s, 1H), 8.88 (d, J=4.8 Hz, 1H), 8.29-8.28 (m, 1H), 8.06 (d, J=4.8 Hz, 1H), 7.64-7.53 (m, 3H), 7.26-7.24 (m, 2H), 4.02-3.94 (m, 2H), 3.91-3.89 (m, 2H), 2.85 (s, 3H), 2.19 (s, 3H). LCMS (M+H⁺) m/z: 513.9

### Example 153: Preparation of N-(4-methyl-3-(2-(methylamino)-8,9-dihydroimidazo[1′,2′:1,6]pyrido[2,3-d]pyrimidin-6-yl)phenyl)-2-oxo-5-(trifluoromethyl)-1,2-dihydropyridine-3-carboxamide (Compound 153)

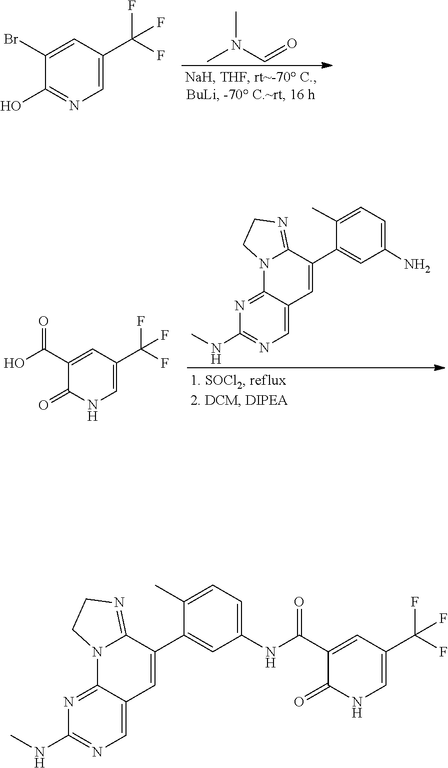

### Step 1: Synthesis of 2-oxo-5-(trifluoromethyl)-1,2-dihydropyridine-3-carboxylic acid

3-Bromo-5-(trifluoromethyl)pyridin-2-ol (960 mg, 4.0 mmol) was added by small portions to a suspension of NaH (180 mg, 4.4 mmol) in anhydrous THF (20 mL). After complete addition of the intermediate, the reaction mixture was cooled to −78° C. and treated with tert-butyllithium (3.2 mL, 8.0 mmol) added dropwise via syringe. After stirring for 5 minutes, DMF (1.0 mL, 12.0 mmol) was added slowly to maintain the temperature below −50° C. The resulting mixture was then stirred for 10 hours allowing warming to room temperature. The mixture was quenched with 2N HCl and then diluted with ethyl acetate (30 mL). The organic layer was separated, washed with brine, dried over MgSO₄, and evaporated in vacuo. The desired product was precipitated out of ethyl acetate and hexane, filtered to yield a light brown solid 2-oxo-5-(trifluoromethyl)-1,2-dihydropyridine-3-carboxylic acid (260 mg, 23.8% yield). 1H NMR (400 MHz, CD₃OD): 10.13 (s, 1H), 8.21 (s, 2H). LCMS (M+H⁺) m/z: 208.0.

### Step 2: Synthesis of N-(4-methyl-3-(2-(methylamino)-8,9-dihydroimidazo[1′,2′:1,6]pyrido[2,3-d]pyrimidin-6-yl)phenyl)-2-oxo-5-(trifluoromethyl)-1,2-dihydropyridine-3-carboxamide

The mixture of 2-oxo-5-(trifluoromethyl)-1,2-dihydropyridine-3-carboxylic acid (37 mg, 0.18 mmol) in SOCl₂ (1.0 mL) was stirred for 2 h at 80° C. The reaction mixture was concentrated and diluted with DCM (2.0 mL), then a solution of DIPEA (58 mg, 0.45 mmol), 6-(5-amino-2-methylphenyl)-N-methyl-8,9-dihydroimidazo[1′,2′:1,6]pyrido[2,3-d]pyrimidin-2-amine (45 mg, 0.15 mmol) in DCM (4.0 mL) was added. The reaction mixture was stirred at 10° C. for 2 h and concentrated and purified by Flash (DCM:MeOH=10:1) and prep-HPLC to give N-(4-methyl-3-(2-(methylamino)-8,9-dihydroimidazo[1′,2′:1,6]pyrido[2,3-d]pyrimidin-6-yl)phenyl)-2-oxo-5-(trifluoromethyl)-1,2-dihydropyridine-3-carboxamide (11.8 mg, 16% yield). 1H NMR (400 MHz, DMSO-d6): 12.12 (s, 1H), 8.53-8.34 (m, 3H), 8.05-8.02 (m, 1H), 7.67-7.58 (m, 3H), 7.31-7.28 (m, 1H), 4.34-4.32 (m, 2H), 3.96-3.92 (m, 2H), 2.90 (s, 3H), 2.19 (s, 3H). LCMS (M+H⁺) m/z: 496.0.

### Example 154: Preparation of N-(4-methyl-3-(2-(methylamino)-8,9-dihydroimidazo[1′,2′:1,6]pyrido[2,3-d]pyrimidin-6-yl)phenyl)-4-(trifluoromethyl)piperidine-2-carboxamide (Compound 154)

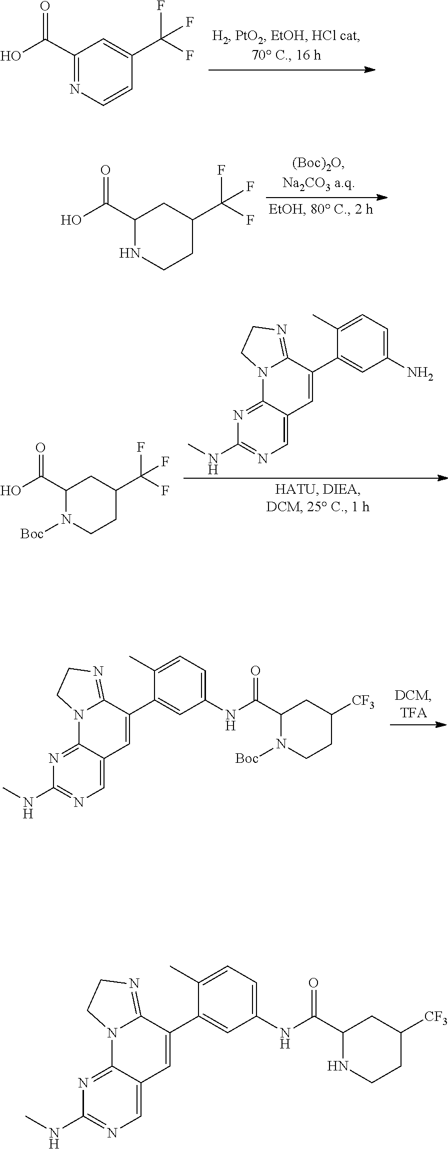

### Step 1: Synthesis of 4-(trifluoromethyl)piperidine-2-carboxylic acid

The mixture of 4-(trifluoromethyl)picolinic acid (191 mg, 1.0 mmol), PtO₂ (45 mg, 0.2 mmol) in MeOH (6.0 mL) and aq HCl (1 drop) was stirred at 70° C. under H2 for 16 h. The reaction mixture was filtered and the filtrate was concentrated to give crude 4-(trifluoromethyl)piperidine-2-carboxylic acid, which was used to next step directly. LCMS (M+H⁺) m/z: 198.1.

### Step 2: Synthesis of 1-(tert-butoxycarbonyl)-4-(trifluoromethyl)piperidine-2-carboxylic acid

The mixture of 4-(trifluoromethyl)piperidine-2-carboxylic acid (200 mg, 1.0 mmol), Boc₂O (260 mg, 1.2 mmol) in MeOH (6.0 mL) and aq Na₂CO₃ (2.0 mL) was stirred at 80° C. for 2 h. pH was adjusted to 6.0 with citric acid aq (4.0 mL). The reaction mixture was extracted with EA (30 mL×2). The combined organic phase was dried over with Na₂SO₄, concentrated to give 1-(tert-butoxycarbonyl)-4-(trifluoromethyl)piperidine-2-carboxylic acid (188 mg, 60% yield) as white solid. LCMS (M+H⁺) m/z: 198.1.

### Step 3: Synthesis of tert-butyl 2-((4-methyl-3-(2-(methylamino)-8,9-dihydroimidazo[1′,2′:1,6]pyrido[2,3-d]pyrimidin-6-yl)phenyl)carbamoyl)-4-(trifluoromethyl)piperidine-1-carboxylate

The mixture of 1-(tert-butoxycarbonyl)-4-(trifluoromethyl)piperidine-2-carboxylic acid (90 mg, 0.3 mmol), HATU (114 mg, 0.3 mmol), DIPEA (97 mg, 0.75 mmol), 6-(5-amino-2-methylphenyl)-N-methyl-8,9-dihydroimidazo[1′,2′:1,6]pyrido[2,3-d]pyrimidin-2-amine (77 mg, 0.25 mmol) in DCM (6.0 mL) was stirred at 10° C. for 2 h. Water was added, the reaction mixture was extracted with EA. The combined extracts were washed with brine. Concentration and purification by flash (DCM:MeOH=20:1) to give tert-butyl 2-((4-methyl-3-(2-(methylamino)-8,9-dihydroimidazo[1′,2′:1,6]pyrido[2,3-d]pyrimidin-6-yl)phenyl)carbamoyl)-4-(trifluoromethyl)piperidine-1-carboxylate (85 mg, 50% yield) as solid. LCMS (M+H⁺) m/z: 586.3.

### Step 4: Synthesis of N-(4-methyl-3-(2-(methylamino)-8,9-dihydroimidazo[1′,2′:1,6]pyrido[2,3-d]pyrimidin-6-yl)phenyl)-4-(trifluoromethyl)piperidine-2-carboxamide

To a mixture of tert-butyl 2-((4-methyl-3-(2-(methylamino)-8,9-dihydroimidazo[1′,2′:1,6]pyrido[2,3-d]pyrimidin-6-yl)phenyl)carbamoyl)-4-(trifluoromethyl)piperidine-1-carboxylate (85 mg, 0.15 mmol) in DCM (3.0 mL), was added TFA (1.0 mL). The mixture was stirred for 1 h at 10° C. The reaction was concentrated, diluted with DCM and water. The pH was adjusted to 8.0 with Na₂CO₃ aq (0.5 mL). the organic phase was concentrated and purified by prep-HPLC (NH₄HCO₃) to give N-(4-methyl-3-(2-(methylamino)-8,9-dihydroimidazo[1′,2′:1,6]pyrido[2,3-d]pyrimidin-6-yl)phenyl)-4-(trifluoromethyl)piperidine-2-carboxamide (12.0 mg, 18% yield). ¹H NMR (400 MHz, DMSO-d₆): 9.75 (s, 1H), 8.26 (s, 1H), 7.52-7.49 (m, 3H), 7.17-7.15 (m, 2H), 4.11-4.02 (m, 2H), 4.00-3.88 (m, 2H), 3.14-3.11 (m, 1H), 2.83 (d, J=11.2 Hz, 3H), 2.67-2.62 (m, 3H), 2.43 (s, 3H), 2.02-2.01 (m, 1H), 1.75-1.74 (m, 1H), 1.35-1.30 (m, 2H). LCMS (M+H⁺) m/z: 486.1.

### Example 155: Preparation of N-(4-methyl-3-(2-(methylamino)-8,9-dihydroimidazo[1′,2′:1,6]pyrido[2,3-d]pyrimidin-6-yl)phenyl)-2-(trifluoromethyl)piperidine-4-carboxamide (Compound 155)

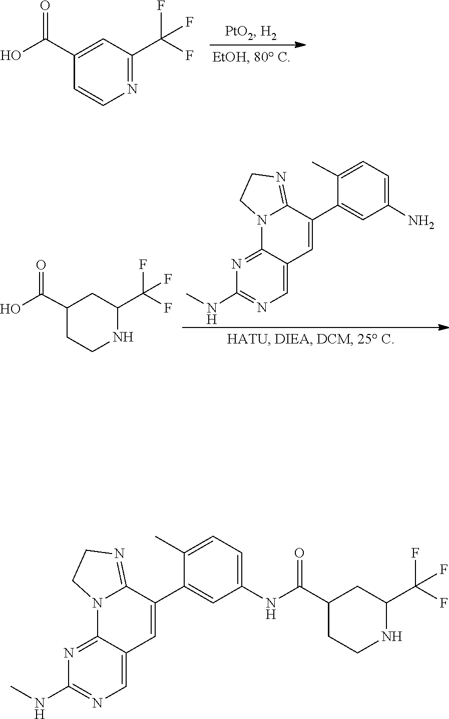

### Step 1: Synthesis of 2-(trifluoromethyl)piperidine-4-carboxylic acid

To a solution of 2-(trifluoromethyl)isonicotinic acid (382 mg, 2 mmol) in EtOH (10 mL) was added PtO₂ (40 mg, 10% w/w %), the reaction mixture was stirred at 80° C. for 4 h under hydrogen atmosphere. LCMS showed the reaction completed. The reaction mixture was filtered by celite, the filtrate was concentrated to obtain of 2-(trifluoromethyl)piperidine-4-carboxylic acid as a white solid (250 mg, crude), which was used for next step without further purification. LCMS (M+H⁺) m/z: 198.0.

### Step 2: Synthesis of N-(4-methyl-3-(2-(methylamino)-8,9-dihydroimidazo[1′,2′:1,6]pyrido[2,3-d]pyrimidin-6-yl)phenyl)-2-(trifluoromethyl)piperidine-4-carboxamide

To a solution of 2-(trifluoromethyl)piperidine-4-carboxylic acid (70 mg, crude) in DCM (5 mL) was added 6-(5-amino-2-methylphenyl)-N-methyl-8,9-dihydroimidazo[1′,2′:1,6]pyrido[2,3-d]pyrimidin-2-amine (110 mg, 0.27 mmol), DIEA (70 mg, 0.54 mmol) and HATU (154 mg. 0.41 mmol). The mixture was stirred at 25° C. for 1 h. LCMS showed the reaction completed. The mixture was diluted with water (10 mL), extracted by DCM (10 mL×3). The combined organic phase was washed by brine (20 mL), dried over anhydrous Na₂SO₄, filtered and concentrated to afford a crude solid, which was purified by prep-HPLC to afford N-(4-methyl-3-(2-(methylamino)-8,9-dihydroimidazo[1′,2′:1,6]pyrido[2,3-d]pyrimidin-6-yl)phenyl)-2-(trifluoromethyl)piperidine-4-carboxamide (19.3 mg) as yellow solid. 1H NMR (400 MHz, DMSO) δ 9.89 (s, 1H), 8.25 (s, 1H), 7.57-7.37 (m, 3H), 7.14 (d, J=8.1 Hz, 2H), 4.04 (d, J=34.6 Hz, 2H), 3.90 (t, J=9.4 Hz, 2H), 3.32 (s, 1H), 3.26 (s, 1H), 3.05 (d, J=11.6 Hz, 1H), 2.84 (s, 3H), 2.58 (d, J=12.5 Hz, 1H), 2.15 (s, 3H), 1.86 (d, J=12.1 Hz, 1H), 1.73 (d, J=11.3 Hz, 1H), 1.45 (dt, J=12.2, 8.4 Hz, 2H). LCMS (M+H⁺) m/z: 486.1.

### Example 156: Preparation of 1-methyl-N-(4-methyl-3-(2-(methylamino)-8,9-dihydroimidazo[1′,2′:1,6]pyrido[2,3-d]pyrimidin-6-yl)phenyl)-2-(trifluoromethyl)piperidine-4-carboxamide (Compound 156)

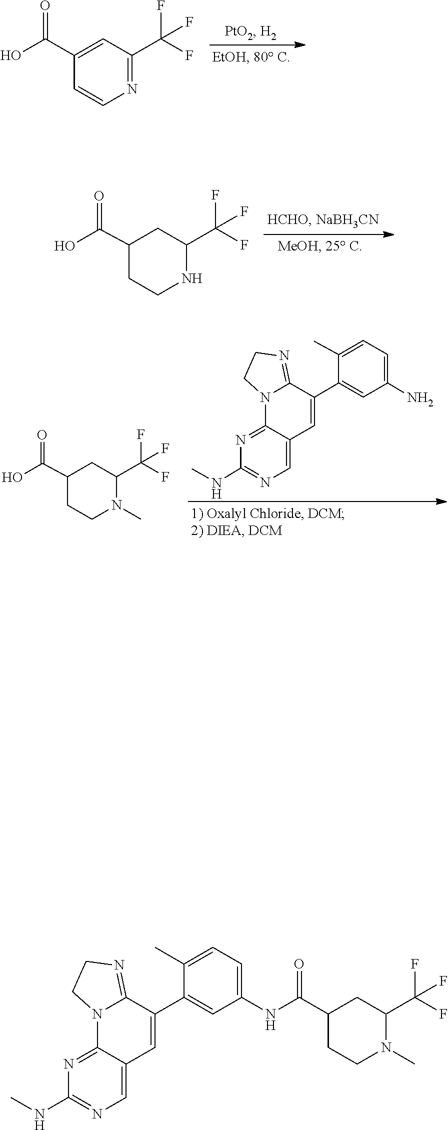

### Step 1: Synthesis of 2-(trifluoromethyl)piperidine-4-carboxylic acid

To a solution of 2-(trifluoromethyl)isonicotinic acid (382 mg, 2 mmol) in EtOH (10 mL) was added PtO₂ (40 mg, 10% w/w %), the reaction mixture was stirred at 80° C. for 4 h under hydrogen atmosphere. LCMS showed the reaction completed. The reaction mixture was filtered by celite, the filtrate was concentrated to obtain 2-(trifluoromethyl)piperidine-4-carboxylic acid as a white solid (250 mg, crude), which was used for next step without further purification. LCMS (M+H⁺) m/z: 198.0.

### Step 2: Synthesis of 1-methyl-2-(trifluoromethyl)piperidine-4-carboxylic acid

To a solution of 2-(trifluoromethyl)piperidine-4-carboxylic acid (80 mg, 0.4 mmol) in MeOH (2 mL) was added HCHO (30% in water) (100 mg, 1 mmol) and NaCNBH3 (126 mg, 2 mmol), the reaction mixture was stirred at 25° C. for 3 h. LCMS showed the reaction completed. The reaction mixture was filtered by celite, the filtrate was concentrated and the residue was diluted with water (10 mL), extracted by EA (10 mL*3), dried over anhydrous Na2SO4, filtered and concentrated to obtain 1-methyl-2-(trifluoromethyl)piperidine-4-carboxylic acid (60 mg, crude), which was used for next step without further purification. LCMS (M+H⁺) m/z: 212.1.

### Step 3: Synthesis of 1-methyl-N-(4-methyl-3-(2-(methylamino)-8,9-dihydroimidazo[1′,2′:1,6]pyrido[2,3-d]pyrimidin-6-yl)phenyl)-2-(trifluoromethyl)piperidine-4-carboxamide

To a solution of 1-methyl-2-(trifluoromethyl)piperidine-4-carboxylic acid (40 mg, crude) in DCM (2 mL) was added oxalyl chloride (127 mg, 1 mmol), the reaction mixture was stirred at 25° C. for 1 h, LCMS showed the reaction completed. The reaction mixture was concentrated to obtain a crude 1-methyl-2-(trifluoromethyl)piperidine-4-carbonyl chloride. To a mixture of 6-(5-amino-2-methylphenyl)-N-methyl-8,9-dihydroimidazo[1′,2′:1,6]pyrido[2,3-d]pyrimidin-2-amine (61 mg, 0.2 mmol) and DIEA (52 mg, 0.4 mmol) in DCM (2 mL) was added the solution of crude 1-methyl-2-(trifluoromethyl)piperidine-4-carbonyl chloride in DCM (1 mL). The mixture was stirred at 25° C. for 1 h. LCMS showed the reaction completed. The mixture was diluted with water (10 mL), extracted by DCM (10 mL×3). The combined organic phase was washed by brine (20 mL), dried over anhydrous Na2SO4, filtered and concentrated to afford a crude solid, which was purified by prep-HPLC to afford 1-methyl-N-(4-methyl-3-(2-(methylamino)-8,9-dihydroimidazo[1′,2′:1,6]pyrido[2,3-d]pyrimidin-6-yl)phenyl)-2-(trifluoromethyl)piperidine-4-carboxamide (12.4 mg, 12% yield) as yellow solid. ¹H NMR (400 MHz, DMSO) δ 9.93 (s, 1H), 8.26 (s, 1H), 8.18 (s, 1H), 7.50 (br, 2H), 7.44 (dd, J=8.4, 2.4 Hz, 1H), 7.20 (br, 1H), 7.15 (d, J=8.4 Hz, 1H), 4.11-4.01 (m, 2H), 3.90 (t, J=9.2 Hz, 2H), 2.92 (d, J=11.6 Hz, 1H), 2.85-2.80 (m, 4H), 2.43 (d, J=12.0 Hz, 1H), 2.30-2.28 (m, 4H), 2.15 (s, 3H), 1.93 (d, J=12.4 Hz, 1H), 1.77 (d, J=12.4 Hz, 1H), 1.60-1.55 (m, 2H). LCMS (M+H⁺) m/z: 500.2.

### Example 157: Preparation of 6-(4-methyl-1H-imidazol-1-yl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (Compound 157)

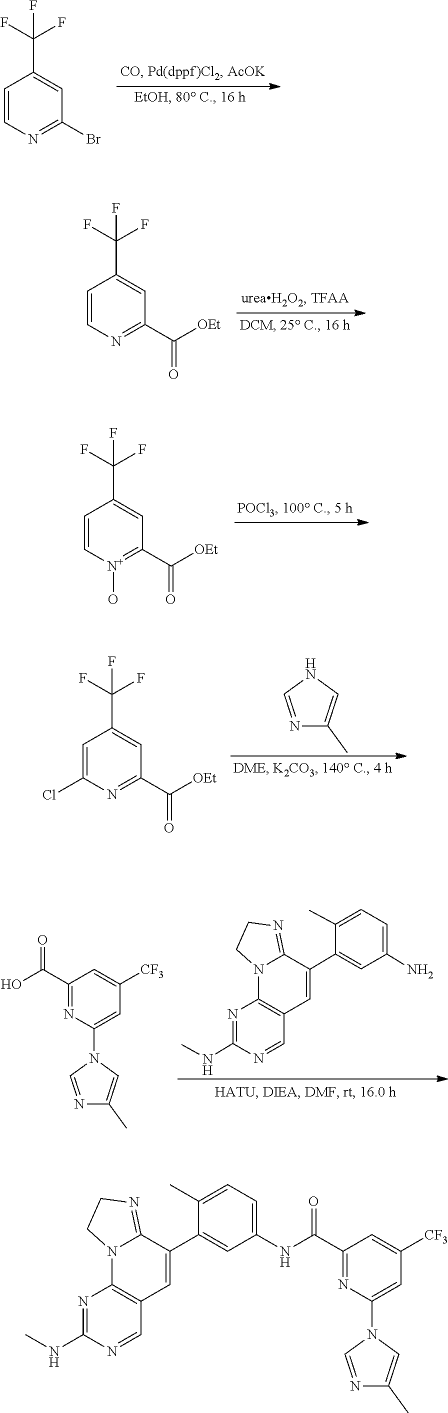

### Step 1: Synthesis of ethyl 4-(trifluoromethyl)picolinate

A mixture of 4-chloro-6-(trifluoromethyl)pyrimidine (2 g, 8.85 mmol), AcOK (3.47 g, 35.4 mmol), and Pd(dppf)Cl₂ (300 mg, 0.5 mmol) in EtOH (30 mL) was degassed and charged with CO for three times and stirred at 80° C. for 16 h under CO. The reaction mixture was concentrated and purified by silica column (PE:EA=3:1) to afford ethyl 4-(trifluoromethyl)picolinate (1.72 g, 88% yield) as brown oil. LCMS (M+H⁺) m/z: 220.0.

### Step 2: Synthesis of 2-(ethoxycarbonyl)-4-(trifluoromethyl)pyridine 1-oxide

A mixture of ethyl 4-(trifluoromethyl)picolinate (1.72 g, 7.8 mmol) and urea hydrogen peroxide (1.48 g, 15.7 mmol) in DCM was added TFAA (3.3 g, 15.7 mmol). The mixture was stirred at rt for 16 h, concentrated and purified by silica column chromatography (PE:EA=3:1) to afford 2-(ethoxycarbonyl)-4-(trifluoromethyl)pyridine 1-oxide (1.8 g, 98% yield) as brown oil. LCMS (M+H⁺) m/z: 236.1.

### Step 3: Synthesis of ethyl 6-chloro-4-(trifluoromethyl)picolinate

A mixture of 2-(ethoxycarbonyl)-4-(trifluoromethyl)pyridine 1-oxide (1.8 g, 7.76 mmol) in POCl₃ (30 mL) was stirred at 100° C. for 5 h. The reaction mixture was concentrated and quenched with NaHCO₃ aq. (10 mL), extracted with EA (20 mL×2). The organic layer was dried with NaSO₄ and filtered, concentrated. The residue was purified by silica column chromatography (PE:EA=3:1) to afford ethyl 6-chloro-4-(trifluoromethyl)picolinate (1.8 g, 92% yield) as brown oil. LCMS (M+H⁺) m/z: 254.2.

### Step 4: Synthesis of 6-(4-methyl-1H-imidazol-1-yl)-4-(trifluoromethyl)picolinic acid

A mixture of ethyl 6-chloro-4-(trifluoromethyl)picolinate (100 mg, 0.40 mmol), 4-methyl-1H-imidazole (66 mg, 0.80 mmol) and K₂CO₃ (109 mg, 0.80 mmol) in DME (5 mL) was stirred at 140° C. for 4 h under N₂. The reaction mixture was concentrated and dissolved in MeOH (5 mL), NaOH (16 mg, 0.40 mmol) was added and the mixture was stirred at rt for 0.5 h. The mixture was diluted with H₂O (20 mL) and the pH was adjusted to 4-5 with HCl (2 M), then was extracted with EA (30 mL×2). The organic layer was dried with NaSO₄, filtered and concentrated in vacuum to afford 6-(4-methyl-1H-imidazol-1-yl)-4-(trifluoromethyl)picolinic acid (80 mg, crude) as brown oil. LCMS (M−H⁺) m/z: 270.1.

### Step 5: Synthesis of 6-(4-methyl-1H-imidazol-1-yl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide

To a mixture of 6-(4-methyl-1H-imidazol-1-yl)-4-(trifluoromethyl)picolinic acid (80 mg, crude), 6-(5-amino-2-methylphenyl)-N-methyl-8,9-dihydroimidazo[1′,2′:1,6]pyrido[2,3-d]pyrimidin-2-amine (90 mg, 0.30 mmol), and HATU (224 mg, 0.59 mmol) in DMF (5.0 mL) was added DIEA (76 mg, 0.59 mmol). The resulting mixture was stirred at r.t. for 16 h under N₂. The reaction mixture was added into water (40.0 mL) slowly, stirred at r.t. for 30 min, filtered. The collected cake was purified by silica column chromatography (DCM:MeOH=15:1, +0.1% NH₃-MeOH) to afford 6-(4-methyl-1H-imidazol-1-yl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (10.2 mg, 6.2% yield) as a yellow solid. ¹H NMR (400 MHz, DMSO-d₆): δ 10.55 (s, 1H), 9.03 (s, 1H), 8.43 (s, 1H), 8.34 (s, 1H), 8.19 (s, 2H), 7.84 (d, J=8.4 Hz, 1H), 7.74 (s, 1H), 7.68-7.48 (m, 1H), 7.32-7.27 (m, 2H), 4.21-4.05 (m, 2H), 3.96-3.94 (t, J=8.8 Hz, 2H), 2.87 (s, 3H), 2.22 (s, 3H), 2.18 (s, 3H). LCMS (M+H⁺) m/z: 560.4.

### Example 158: Preparation of 5-((4-ethylpiperazin-1-yl)methyl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (Compound 158)

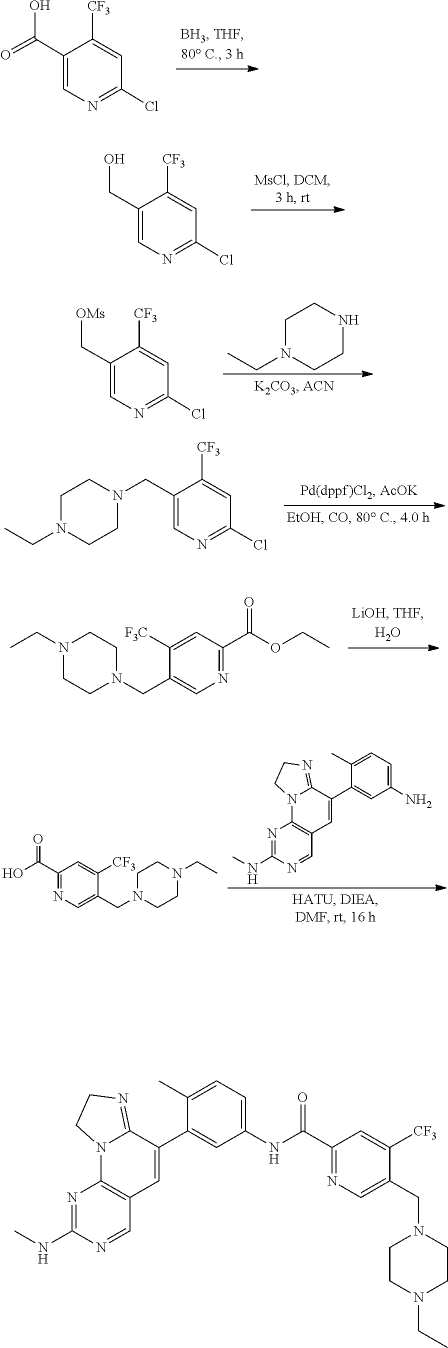

### Step 1: Synthesis of (6-chloro-4-(trifluoromethyl)pyridin-3-yl)methanol

A mixture of 6-chloro-4-(trifluoromethyl)nicotinic acid (400 mg, 1.78 mmol) in THF (10 mL) was added BH₃·THF (2M, 1.78 mL) dropwise at rt. The mixture was stirred at 80° C. for 4 hours. The reaction mixture was quenched with water (20 mL) and extracted with DCM (20 mL). The organic phase was concentrated in vacuum and the residue was purified by column chromatography on silica column chromatography (PE:EA=4:1) to afford (6-chloro-4-(trifluoromethyl)pyridin-3-yl)methanol (360 mg, 96% yield) as yellow oil. LCMS (M+H⁺) m/z: 212.0.

### Step 2: Synthesis of (6-chloro-4-(trifluoromethyl)pyridin-3-yl)methyl methanesulfonate

A mixture of (6-chloro-4-(trifluoromethyl)pyridin-3-yl)methanol (200 mg, 0.95 mmol) in THF (10 mL) was added MsCl (218 mg, 1.895 mmol) dropwise at 0° C. The mixture was stirred at 30° C. for 4 hours. The reaction mixture was diluted with DCM (20 mL) and washed with brine (10 mL×2). The organic phase was dried with NaSO₄, filtered, concentrated in vacuum to give (6-chloro-4-(trifluoromethyl)pyridin-3-yl)methyl methanesulfonate (240 mg, crude) as a yellow solid. LCMS (M+H⁺) m/z: 289.8.

### Step 3: Synthesis of 1-((6-chloro-4-(trifluoromethyl)pyridin-3-yl)methyl)-4-ethylpiperazine

A mixture of (6-chloro-4-(trifluoromethyl)pyridin-3-yl)methyl methanesulfonate (240 mg, crude) and K₂CO₃ (433 mg, 3.80 mmol) in CH₃CN (10 mL) was added 1-ethylpiperazine (197 mg, 1.425 mmol). The mixture was stirred at 80° C. for 4 hours and concentrated. The residue was purified by column chromatography on silica gel (DCM:MeOH=10:1) to afford 1-((6-chloro-4-(trifluoromethyl)pyridin-3-yl)methyl)-4-ethylpiperazine (180 mg, 51% yield) as brown oil. LCMS (M+H⁺) m/z: 308.1.

### Step 4: Synthesis of ethyl 5-((4-ethylpiperazin-1-yl)methyl)-4-(trifluoromethyl)picolinate

A mixture of 1-((6-chloro-4-(trifluoromethyl)pyridin-3-yl)methyl)-4-ethylpiperazine (180 mg, 0.59 mmol), AcOK (230 mg, 2.35 mmol) and Pd(dppf)Cl₂ (21 mg, 0.03 mmol) in EtOH (20 mL) was degassed and charged with CO three times and stirred at 80° C. for 16 h under CO. The reaction mixture was concentrated and purified by column chromatography on silica gel (DCM:MeOH=15:1) to afford ethyl 5-((4-ethylpiperazin-1-yl)methyl)-4-(trifluoromethyl)picolinate (180 mg, 88% yield) as a yellow solid. LCMS (M+H⁺) m/z: 346.2.

### Step 5: Synthesis of 5-((4-ethylpiperazin-1-yl)methyl)-4-(trifluoromethyl)picolinic acid

LiOH (6.9 mg, 1.45 mmol) was added to the mixture of ethyl 5-((4-ethylpiperazin-1-yl)methyl)-4-(trifluoromethyl)picolinate (100 mg, 0.29 mmol) in THF:H₂O (5:1) (5 mL). The mixture was stirred at r.t. for 2 hours. The reaction mixture was quenched with water (20 mL) and extracted with DCM (20 mL). The aqueous phase was adjusted to pH=3 with 2N HCl and then concentrated to afford concentrated 5-((4-ethylpiperazin-1-yl)methyl)-4-(trifluoromethyl)picolinic acid (150 mg, crude) as a white solid. LCMS (M+H⁺) m/z: 318.2.

### Step 6: Synthesis of 5-((4-ethylpiperazin-1-yl)methyl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide

To the mixture of 5-((4-Ethylpiperazin-1-yl)methyl)-4-(trifluoromethyl)picolinic acid (25 mg, 0.078 mmol), 6-(5-amino-2-methylphenyl)-N-methyl-8,9-dihydroimidazo[1′,2′:1,6]pyrido[2,3-d]pyrimidin-2-amine (20 mg, 0.065 mmol), DIEA (25 mg, 0.20 mmol) in DMF (1 mL) was added HATU (37 mg, 0.098 mmol). The mixture was stirred at r.t for 16 hours. The reaction mixture was quenched with water (30 mL) and extracted with DCM (30 mL×3), The combined organic layers were washed with brine (60 mL), dried over Na₂SO₄, filtered, concentrated to afford crude which was purified by Prep-HPLC (0.1% NH₃·H₂O) to afford 5-((4-ethylpiperazin-1-yl)methyl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (12.9 mg, 32.6% yield) as a yellow solid. ¹H NMR (400 MHz, DMSO-d₆): δ 10.70 (s, 1H), 9.05 (s, 1H), 8.26 (s, 1H), 8.22 (t, J=7.2 Hz, 2H), 7.80 (s, 1H), 7.74 (t, J=8.0 Hz, 1H), 7.39-7.38 (m, 1H), 7.22 (d, J=8.4 Hz, 1H), 7.11 (s, 1H), 4.02-3.93 (m, 2H), 3.91-3.89 (m, 2H), 3.77 (s, 2H), 2.84 (d, J=4.0 Hz, 3H), 2.45-2.30 (m, 7H), 2.28-2.20 (m, 3H) 2.07 (s, 3H), 0.98 (t, J=7.2 Hz, 3H). LCMS (M+H⁺) m/z: 606.3.

### Example 159: Preparation of 4-cyano-N-(4-methyl-3-(2-(methylamino)-8,9-dihydroimidazo[1′,2′:1,6]pyrido[2,3-d]pyrimidin-6-yl)phenyl)picolinamide (Compound 159)

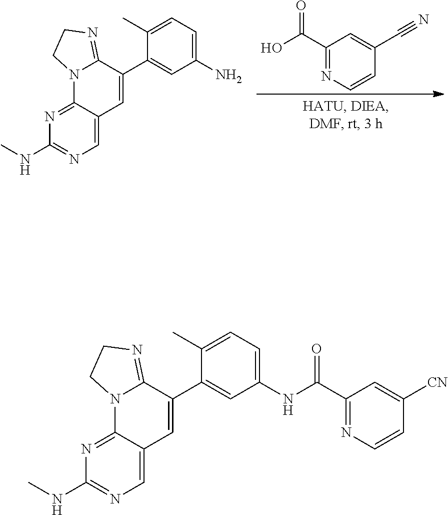 [SMILES: CNc1ncc2c(n1)N1CCN=C1C(c1cc(N)ccc1C)=C2 ; N#Cc1ccnc(C(=O)O)c1 ; CNc1ncc2c(n1)N1CCN=C1C(c1cc(NC(=O)c3cc(C#N)ccn3)ccc1C)=C2]

### Step 1: Synthesis of 4-cyano-N-(4-methyl-3-(2-(methylamino)-8,9-dihydroimidazo[1′,2′:1,6]pyrido[2,3-d]pyrimidin-6-yl)phenyl)picolinamide

A mixture of 6-(5-amino-2-methylphenyl)-N-methyl-8,9-dihydroimidazo[1′,2′:1,6]pyrido[2,3-d]pyrimidin-2-amine (200 mg, 0.65 mmol), 4-cyanopicolinic acid (120 mg, 0.78 mmol), DIEA (151 mg, 1.17 mmol) and HATU (321 mg, 0.85 mmol) in DMF (10.0 mL) was stirred at r.t. for 16 h. The reaction mixture was diluted with water (20.0 mL) and extracted with EA (50 mL×2). The combined organic phase was washed with brine (10 mL×3), dried with Na2SO4, filtered and concentrated. The residue was purified by Prep-HPLC (0.1% NH3H2O) to afford 4-cyano-N-(4-methyl-3-(2-(methylamino)-8,9-dihydroimidazo[1′,2′:1,6]pyrido[2,3-d]pyrimidin-6-yl)phenyl)picolinamide (100 mg, 35% yield) as a yellow solid. ¹H NMR (400 MHz, DMSO-d₆): δ 10.71 (s, 1H), 8.98 (d, J=4.8 Hz, 1H), 8.46 (s, 1H), 8.25 (br, 1H), 8.15 (dd, J=4.8, 1.2 Hz, 1H), 7.82 (d, J=2.0 Hz, 1H), 7.75 (dd, J=8.0, 2.0 Hz, 1H), 7.51-7.45 (m, 1H), 7.24-7.18 (m, 2H), 4.10-3.89 (m, 4H), 2.81 (s, 3H), 2.20 (s, 3H). LCMS (M+H⁺) m/z: 437.5.

### Example 160: Preparation of 5-chloro-N-(4-methyl-3-(2-(methylamino)-8,9-dihydroimidazo[1′,2′:1,6]pyrido[2,3-d]pyrimidin-6-yl)phenyl)nicotinamide (Compound 160)

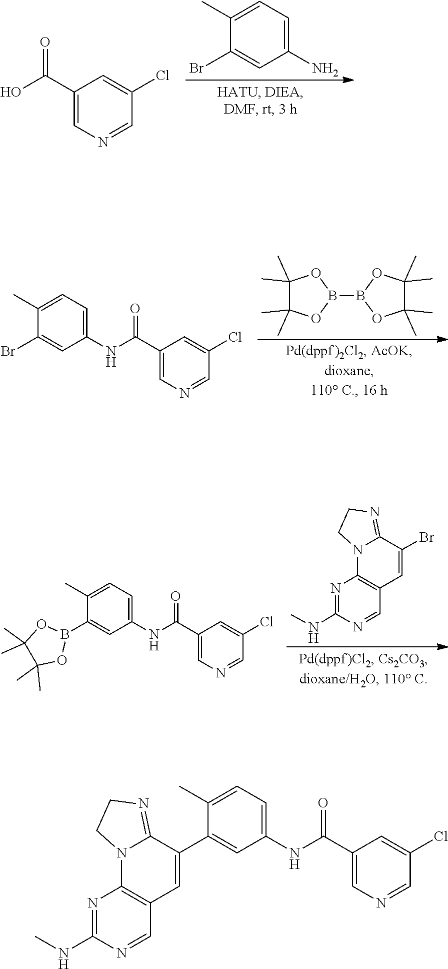

### Step 1: Synthesis of N-(3-bromo-4-methylphenyl)-5-chloronicotinamide

A mixture of 5-chloronicotinic acid (1 g, 6.36 mmol), 3-bromo-4-methylaniline (1.2 g, 6.4 mmol), HATU (2.7 g, 7.7 mmol) and DIEA (1.23 g, 9.54 mmol) in DMF (30 mL) at RT. The reaction mixture was stirred at RT for 2 h. The reaction mixture was diluted with water (80 mL), extracted with EA (100 mL×3). The combined organic phase was washed with brine (50 mL×3), dried with Na₂SO₄, filtered and concentrated. The residue was purified by column chromatography (PE/EA=10/1) to afford N-(3-bromo-4-methylphenyl)-5-chloronicotinamide (1.5 g, 47% yield).

### Step 2: Synthesis of 5-chloro-N-(4-methyl-3-(4,4,5,5-tetramethyl-1,3,2-dioxaborolan-2-yl)phenyl)nicotinamide

A mixture of N-(3-bromo-4-methylphenyl)-5-chloronicotinamide (325 mg, 1 mmol) in dioxane (5 mL) was added 4,4,4′,4′,5,5,5′,5′-octamethyl-2,2′-bi(1,3,2-dioxaborolane) (280 mg, 1.2 mmol), KOAc (650 mg, 6.6 mmol), Pd(dppf)Cl2 (160 mg, 0.22 mmol). The mixture was degassed and charged with Ar for 3 times, stirred at 100° C. for 3 h. The reaction mixture was concentrated under vacuum and H₂O (50 mL) was added. The reaction mixture was extracted with EA (100 mL×3). The combined organic phases were washed with brine (20 mL), dried over Na2SO4, filtered and concentrated. The residue was purified by column chromatography (PE/EA=10/1) to afford 5-chloro-N-(4-methyl-3-(4,4,5,5-tetramethyl-1,3,2-dioxaborolan-2-yl)phenyl)nicotinamide (300 mg, 81% yield). LCMS (M+H+) m/z: 373.0.

### Step 3: Synthesis of 5-chloro-N-(4-methyl-3-(2-(methylamino)-8,9-dihydroimidazo[1′,2′:1,6]pyrido[2,3-d]pyrimidin-6-yl)phenyl)nicotinamide

A mixture of 6-bromo-N-methyl-8,9-dihydroimidazo[1′,2′:1,6]pyrido[2,3-d]pyrimidin-2-amine (100 mg, 0.36 mmol), 5-chloro-N-(4-methyl-3-(4,4,5,5-tetramethyl-1,3,2-dioxaborolan-2-yl)phenyl)nicotinamide (200 mg, 0.54 mmol), Cs₂CO₃ (235 mg, 0.72 mmol) and Pd(dppf)Cl₂ (58 mg, 0.07 mmol) in dioxane (15.0 mL) and water (3 mL) was degassed, charged with N₂ for three times and stirred at 100° C. for 16 h. The reaction mixture was cooled to r.t., diluted with water (20 mL) and extracted with EA (50 mL×3). The combined organic phase was washed with brine (100 mL), dried with Na₂SO₄, filtered and concentrated. The residue was purified by Prep-HPLC (0.1% HCl) to afford 5-chloro-N-(4-methyl-3-(2-(methylamino)-8,9-dihydroimidazo[1′,2′:1,6]pyrido[2,3-d]pyrimidin-6-yl)phenyl)nicotinamide (10 mg, 5% yield) as a yellow solid. ¹H NMR (400 MHz, DMSO-d₆): δ 10.90 (s, 1H), 9.77 (s, 1H), 9.09 (s, 1H), 8.98-8.84 (m, 2H), 8.53-8.50 (m, 2H), 8.14 (s, 1H), 7.89 (s, 1H), 7.81 (d, J=8.0 Hz, 1H), 7.39 (d, J=8.4 Hz, 1H), 4.67-4.54 (m, 2H), 4.10-4.05 (m, 2H), 2.96 (s, 3H), 2.20 (s, 3H). LCMS (M+H⁺) m/z: 446.1.

### Example 161: Preparation of 3-(2-cyanopropan-2-yl)-N-(4-methyl-3-(2-(methylamino)-8,9-dihydroimidazo[1′,2′:1,6]pyrido[2,3-d]pyrimidin-6-yl)phenyl)benzamide (Compound 161)

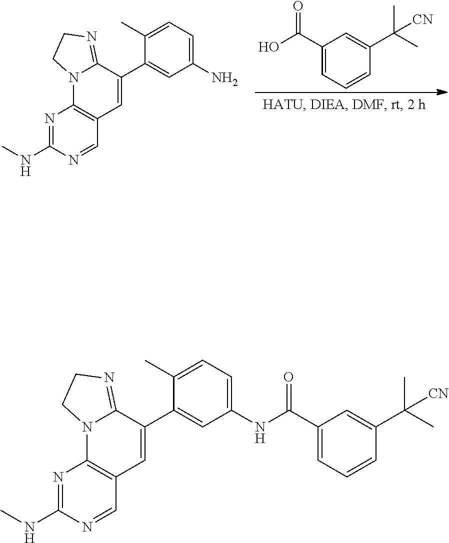 [SMILES: CNc1ncc2c(n1)N1CCN=C1C(c1cc(N)ccc1C)=C2 ; CC(C)(C#N)c1cccc(C(=O)O)c1 ; CNc1ncc2c(n1)N1CCN=C1C(c1cc(NC(=O)c3cccc(C(C)(C)C#N)c3)ccc1C)=C2]

### Step 1: Synthesis of 3-(2-cyanopropan-2-yl)-N-(4-methyl-3-(2-(methylamino)-8,9-dihydroimidazo[1′,2′:1,6]pyrido[2,3-d]pyrimidin-6-yl)phenyl)benzamide

A mixture of 6-(5-amino-2-methylphenyl)-N-methyl-8,9-dihydroimidazo[1′,2′:1,6]pyrido[2,3-d]pyrimidin-2-amine (90 mg, 0.29 mmol), 3-(2-cyanopropan-2-yl)benzoic acid (55 mg, 0.29 mmol), HATU (168 mg, 0.48 mmol) and DIEA (114 mg, 0.88 mmol) in DMF (6 mL) was stirred at RT for 2 h. The reaction mixture was diluted with water (20 mL), extracted with EA (50 mL×3). The combined organic phase was washed with brine (50 mL×3), dried with Na₂SO₄, filtered and concentrated. The residue was purified by Prep-HPLC (0.1% FA) to afford 3-(2-cyanopropan-2-yl)-N-(4-methyl-3-(2-(methylamino)-8,9-dihydroimidazo[1′,2′:1,6]pyrido[2,3-d]pyrimidin-6-yl)phenyl)benzamide (13.4 mg, 9.6% yield) as a yellow solid. ¹H NMR (400 MHz, DMSO-d₆): δ 10.28 (s, 1H), 8.27 (s, 1H), 8.19 (s, 1H), 8.05 (s, 1H), 7.95 (d, J=7.6 Hz, 1H), 7.76 (d, J=7.6 Hz, 1H), 7.69 (d, J=8.4 Hz, 1H), 7.58-7.64 (m, 2H), 7.44-7.53 (m, 1H), 7.22-7.25 (m, 2H), 4.02-4.11 (m, 2H), 3.90-3.95 (m, 2H), 2.86 (s, 3H), 2.20 (s, 3H), 1.76 (s, 6H). LCMS (M+H⁺) m/z: 478.3.

### Example 162: Preparation of 2-(2-cyanopropan-2-yl)-N-(4-methyl-3-(2-(methylamino)-8,9-dihydroimidazo[1′,2′:1,6]pyrido[2,3-d]pyrimidin-6-yl)phenyl)isonicotinamide (Compound 162)

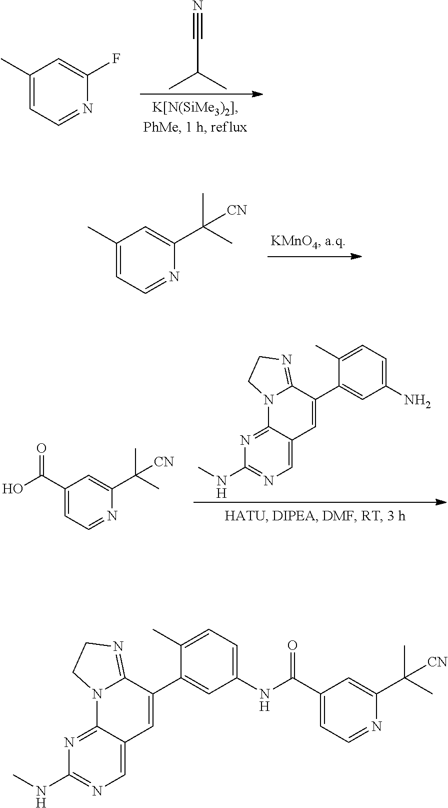

### Step 1: Synthesis of 2-methyl-2-(4-methylpyridin-2-yl)propanenitrile

To a solution of 2-fluoro-4-methylpyridine (1.11 g, 10 mmol) in toluene (10 mL) was added isobutyronitrile (1.38 g, 20 mmol) and KHMDS (1M in toluene) (20 mL). The mixture was stirred at 110° C. for 1 h. LCMS showed the reaction completed. The reaction mixture was diluted to water (10 mL), extracted by EA (10 mL×3). The combined organic phase was washed by brine (20 mL), dried over anhydrous Na2SO4, concentrated to obtain 2-methyl-2-(4-methylpyridin-2-yl)propanenitrile (630 mg, 40% yield). LCMS (M+H⁺) m/z: 161.0.

### Step 2: Synthesis of 2-(2-cyanopropan-2-yl)isonicotinic acid

To a solution of 2-methyl-2-(4-methylpyridin-2-yl)propanenitrile (320 mg, 2 mmol) in water (5 mL) was added KMnO4 (632 mg, 4 mmol). The mixture was stirred at 90° C. for 3 h. LCMS showed the reaction completed. The reaction mixture was diluted to water (10 mL), extracted by EA (10 mL×3). The combined organic phase was washed by brine (20 mL), dried over anhydrous Na2SO4, concentrated to afford 2-(2-cyanopropan-2-yl)isonicotinic acid (180 mg, 47% yield). LCMS (M+H⁺) m/z: 191.0.

### Step 3: Synthesis of 2-(2-cyanopropan-2-yl)-N-(4-methyl-3-(2-(methylamino)-8,9-dihydroimidazo[1′,2′:1,6]pyrido[2,3-d]pyrimidin-6-yl)phenyl)isonicotinamide

To a solution of 2-(2-cyanopropan-2-yl)isonicotinic acid (40 mg, 0.21 mmol) in DCM (5 mL) was added 6-(5-amino-2-methylphenyl)-N-methyl-8,9-dihydroimidazo[1′,2′:1,6]pyrido[2,3-d]pyrimidin-2-amine (64 mg, 0.21 mmol), DIEA (81 mg, 0.63 mmol) and HATU (118 mg, 0.31 mmol). The mixture was stirred at 25° C. for 2 h. LCMS showed the reaction completed. The reaction mixture was diluted to water (10 mL), extracted by DCM (10 mL×3). The combined organic phase was washed by brine (20 mL), dried over anhydrous Na2SO4, concentrated to get a crude solid, which was purified by prep-HPLC to obtain 2-(2-cyanopropan-2-yl)-N-(4-methyl-3-(2-(methylamino)-8,9-dihydroimidazo[1′,2′:1,6]pyrido[2,3-d]pyrimidin-6-yl)phenyl)isonicotinamide (3.8 mg, 4% yield) as yellow solid. ¹H NMR (400 MHz, DMSO-d6): δ 10.53 (s, 1H), 8.80 (d, J=5.2 Hz, 1H), 8.30-8.21 (m, 1H), 8.01 (s, 1H), 7.86 (d, J=5.2 Hz, 1H), 7.67 (dd, J=8.4, 2.4 Hz, 1H), 7.61 (d, J=2.0 Hz, 1H), 7.43 (s, 1H), 7.24 (d, J=8.4 Hz, 1H), 7.12 (s, 1H), 6.10 (s, 1H), 4.04-3.88 (m, 4H), 2.84 (s, 3H), 2.20 (s, 3H), 1.77 (s, 6H). LCMS (M+H+) m/z: 479.0.

### Example 163: Preparation of 4-(2-cyanopropan-2-yl)-N-(4-methyl-3-(2-(methylamino)-8,9-dihydroimidazo[1′,2′:1,6]pyrido[2,3-d]pyrimidin-6-yl)phenyl)picolinamide (Compound 163)

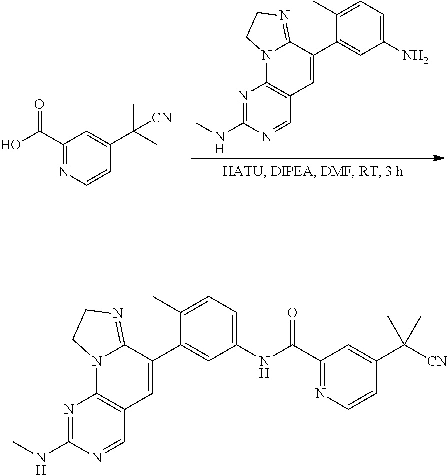

### Step 1: Synthesis of 4-(2-cyanopropan-2-yl)-N-(4-methyl-3-(2-(methylamino)-8,9-dihydroimidazo[1′,2′:1,6]pyrido[2,3-d]pyrimidin-6-yl)phenyl)picolinamide

To a solution of 6-(5-amino-2-methylphenyl)-N-methyl-8,9-dihydroimidazo[1′,2′:1,6]pyrido[2,3-d]pyrimidin-2-amine (80 mg, 0.26 mmol), 4-(2-cyanopropan-2-yl)picolinic acid (50 mg, 0.26 mmol), HATU (150 mg, 0.39 mmol) and DIEA (101 mg, 0.78 mmol) in DMF (5 mL) at RT. The reaction mixture was stirred at RT for 2 h. The reaction mixture was diluted with water (20 mL), filtered and the filter cake was purified by column chromatography (DCM:MeOH=20:1) to afford 4-(2-cyanopropan-2-yl)-N-(4-methyl-3-(2-(methylamino)-8,9-dihydroimidazo[1′,2′:1,6]pyrido[2,3-d]pyrimidin-6-yl)phenyl)picolinamide (68.9 mg, 55.1% yield) as an off-white solid. 1H NMR (400 MHz, DMSO-d6): δ 10.78 (s, 1H), 8.97 (s, 1H), 8.88 (s, 1H), 8.81 (d, J=5.2 Hz, 1H), 8.26 (d, J=1.2 Hz, 1H), 8.12 (m, J=2.4 Hz, 1H), 7.94-7.90 (m, 2H), 7.85-7.83 (m, 1H), 7.42 (d, J=8.8 Hz, 1H), 4.69-4.56 (m, 2H), 4.06-4.01 (m, 2H), 2.97 (s, 3H), 2.21 (s, 3H), 1.76 (s, 6H). LCMS (M+H⁺) m/z: 479.3.

### Example 164: Preparation of N-(3-(2-(azetidin-3-ylamino)-8,9-dihydroimidazo[1′,2′:1,6]pyrido[2,3-d]pyrimidin-6-yl)-4-methylphenyl)-4-(2-cyanopropan-2-yl)picolinamide (Compound 164)

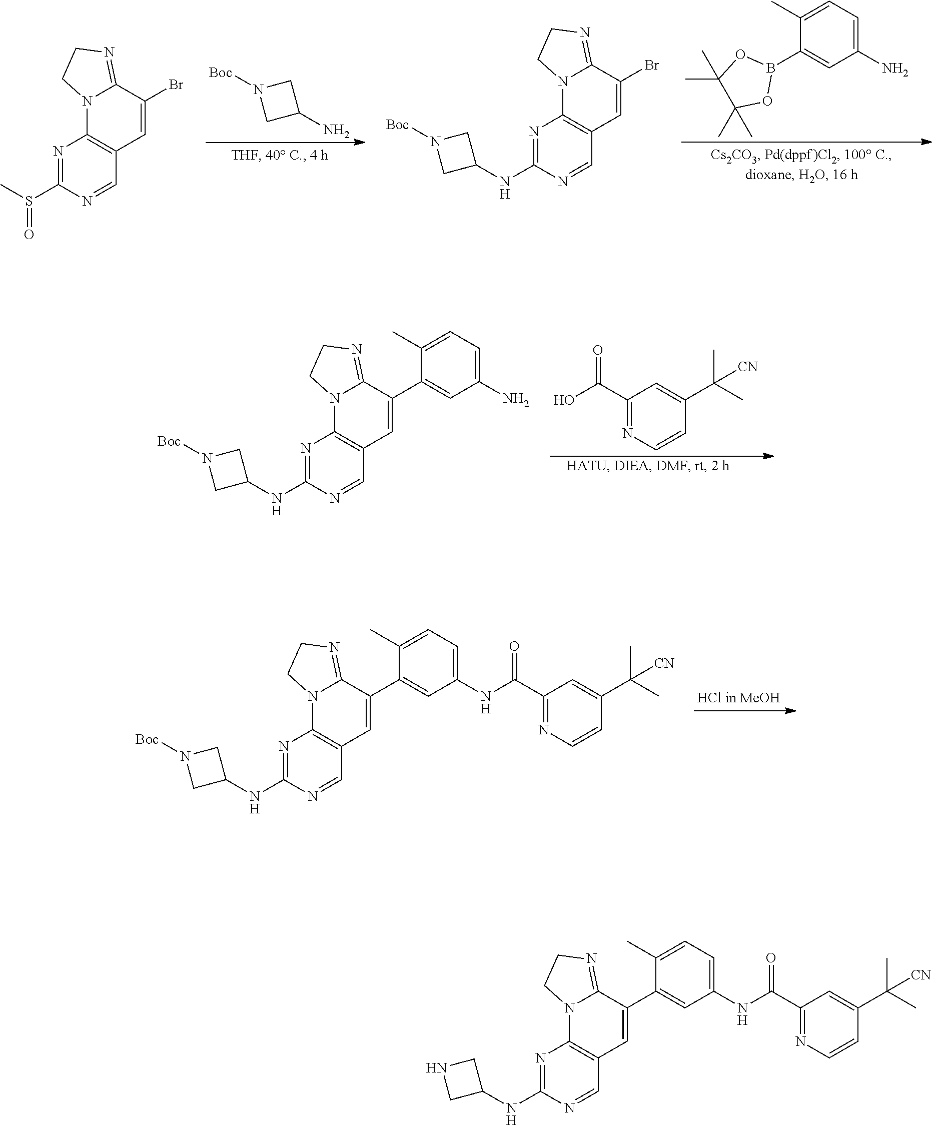

### Step 1: Synthesis of tert-butyl 3-((6-bromo-8,9-dihydroimidazo[1′,2′:1,6]pyrido[2,3-d]pyrimidin-2-yl)amino)azetidine-1-carboxylate

A mixture of tert-butyl 3-aminoazetidine-1-carboxylate (165 mg 0.96 mmol), 6-bromo-2-(methylsulfinyl)-8,9-dihydroimidazo[1′,2′:1,6]pyrido[2,3-d]pyrimidine (150 mg, 0.48 mmol) in dry THF (5 mL) was stirred at 40° C. for 6 h. The reaction mixture was concentrated in vacuum and purified by column chromatography on silica gel (DCM/MeOH=15/1) to afford tert-butyl 3-((6-bromo-8,9-dihydroimidazo[1′,2′:1,6]pyrido[2,3-d]pyrimidin-2-yl)amino)azetidine-1-carboxylate (150 mg, 96.5% yield) as a yellow solid. LCMS (M+H⁺) m/z: 421.1 and 423.1.

### Step 2: Synthesis of tert-butyl 3-((6-(5-amino-2-methylphenyl)-8,9-dihydroimidazo[1′,2′:1,6]pyrido[2,3-d]pyrimidin-2-yl)amino)azetidine-1-carboxylate

A mixture of tert-butyl 3-((6-(5-amino-2-methylphenyl)-8,9-dihydroimidazo[1′,2′:1,6]pyrido[2,3-d]pyrimidin-2-yl)amino)azetidine-1-carboxylate (150 mg, 0.356 mmol), 4-methyl-3-(4,4,5,5-tetramethyl-1,3,2-dioxaborolan-2-yl)aniline (91 mg, 0.392 mmol), Cs₂CO₃ (348 mg, 1.069 mmol) and Pd(dppf)Cl₂ (14 mg, 0.02 mmol) in dioxane (5 mL) and water (0.5 mL) was degassed, charged with N₂ three times and stirred at 100° C. for 16 h. The reaction mixture was concentrated in vacuum and purified by column chromatography on silica gel chromatography (DCM/MeOH=10/1) to afford tert-butyl 3-((6-(5-amino-2-methylphenyl)-8,9-dihydroimidazo[1′,2′:1,6]pyrido[2,3-d]pyrimidin-2-yl)amino)azetidine-1-carboxylate (120 mg, 75.4% yield) as a yellow solid. LCMS (M+H⁺) m/z: 448.3.

### Step 3: Synthesis of tert-butyl 3-((6-(5-(4-(2-cyanopropan-2-yl)picolinamido)-2-methylphenyl)-8,9-dihydroimidazo[1′,2′:1,6]pyrido[2,3-d]pyrimidin-2-yl)amino)azetidine-1-carboxylate

To a solution of tert-butyl 3-((6-(5-amino-2-methylphenyl)-8,9-dihydroimidazo[1′,2′:1,6]pyrido[2,3-d]pyrimidin-2-yl)amino)azetidine-1-carboxylate (80 mg, 0.179 mmol), 4-(2-cyanopropan-2-yl)picolinic acid (37 mg, 0.196 mmol) and HATU (136 mg, 0.357 mmol) in DMF (5.0 mL) was added DIEA (46 mg, 0.357 mmol). The resulting mixture was stirred at r.t. for 2 h under N₂. The reaction mixture was quenched with water (30 mL) and extracted with DCM (30 mL×3). The combined organic layers were washed with brine (30 mL), dried over Na2SO4, filtered, concentrated to afford the crude product, which was purified by column chromatography on silica gel (DCM/MeOH=10/1) to afford tert-butyl 3-((6-(5-(4-(2-cyanopropan-2-yl)picolinamido)-2-methylphenyl)-8,9-dihydroimidazo[1′,2′:1,6]pyrido[2,3-d]pyrimidin-2-yl)amino)azetidine-1-carboxylate (100 mg, 77.7% yield) as brown solid. LCMS (M+H⁺) m/z: 620.9.

### Step 4: Synthesis of N-(3-(2-(azetidin-3-ylamino)-8,9-dihydroimidazo[1′,2′:1,6]pyrido[2,3-d]pyrimidin-6-yl)-4-methylphenyl)-4-(2-cyanopropan-2-yl)picolinamide

To a solution of tert-butyl 3-((6-(5-(4-(2-cyanopropan-2-yl)picolinamido)-2-methylphenyl)-8,9-dihydroimidazo[1′,2′:1,6]pyrido[2,3-d]pyrimidin-2-yl)amino)azetidine-1-carboxylate (40 mg, 0.065 mmol) in HCl-MeOH (1 M, 5.0 mL) was stirred at r.t. for 1 h. Then the pH was adjusted to 8-9 with NH₃—H₂O, the reaction mixture was concentrated and purified by Prep-HPLC (0.1% NH₃—H₂O) to afford N-(3-(2-(azetidin-3-ylamino)-8,9-dihydroimidazo[1′,2′:1,6]pyrido[2,3-d]pyrimidin-6-yl)-4-methylphenyl)-4-(2-cyanopropan-2-yl)picolinamide (30.0 mg, 89.5% yield) as a yellow solid. ¹H NMR (400 MHz, DMSO-d₆): δ 10.62 (s, 1H), 8.78 (d, J=5.2 Hz, 1H), 8.25-8.21 (m, 2H), 8.08-7.97 (m, 1H), 7.74-7.35 (m, 3H), 7.21 (d, J=8.4 Hz, 1H), 7.12 (s, 1H), 4.71-4.70 (m, 1H), 4.11-3.80 (m, 4H), 3.63-3.33 (m, 4H), 2.20 (s, 3H), 1.76 (s, 6H). LCMS (M+H⁺) m/z: 520.4.

### Example 165: Preparation of 4-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)picolinamide (Compound 165)

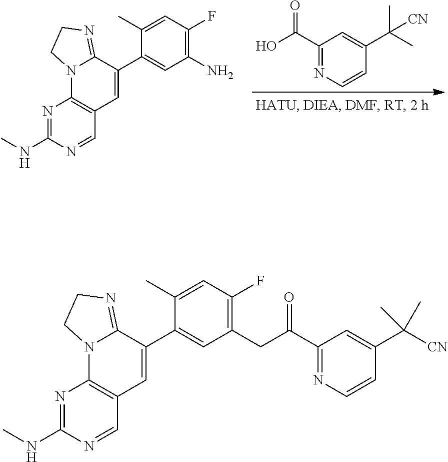 [SMILES: CNc1ncc2c(n1)N1CCN=C1C(c1cc(N)c(F)cc1C)=C2 ; CC(C)(C#N)c1ccnc(C(=O)O)c1 ; CNc1ncc2c(n1)N1CCN=C1C(c1cc(CC(=O)c3cc(C(C)(C)C#N)ccn3)c(F)cc1C)=C2]

The preparation of 6-(5-amino-4-fluoro-2-methylphenyl)-N-methyl-8,9-dihydroimidazo[1′,2′:1,6]pyrido[2,3-d]pyrimidin-2-amine was described in Example 139.

### Step 1: 4-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)picolinamide

To a solution of 6-(5-amino-4-fluoro-2-methylphenyl)-N-methyl-8,9-dihydroimidazo[1′,2′:1,6]pyrido[2,3-d]pyrimidin-2-amine (70 mg, 0.22 mmol), 4-(2-cyanopropan-2-yl)picolinic acid (43 mg, 0.22 mmol), HATU (123 mg, 0.32 mmol) and DIEA (84 mg, 0.65 mmol) in DMF (5 mL) was stirred at RT for 2 h. The reaction mixture was diluted with water (20 mL), filtered and the filter cake was purified by column chromatography on silica gel (DCM:MeOH=20:1) to afford 4-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)picolinamide (19.0 mg, 55.1% yield) as an off-white solid. ¹H NMR (400 MHz, DMSO-d₆): δ 10.39 (s, 1H), 8.82 (d, J=5.6 Hz, 1H), 8.29-8.25 (m, 2H), 7.92 (d, J=8.0 Hz, 1H), 7.88-7.86 (m, 1H), 7.59-7.49 (m, 1H), 7.27-7.24 (m, 2H), 4.14-4.03 (m, 2H), 3.95-3.90 (m, 2H), 2.86 (s, 3H), 2.23 (s, 3H), 1.76 (s, 6H). LCMS (M+H⁺) m/z: 497.5.

### Example 166: Preparation of 2-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)isonicotinamide (Compound 166)

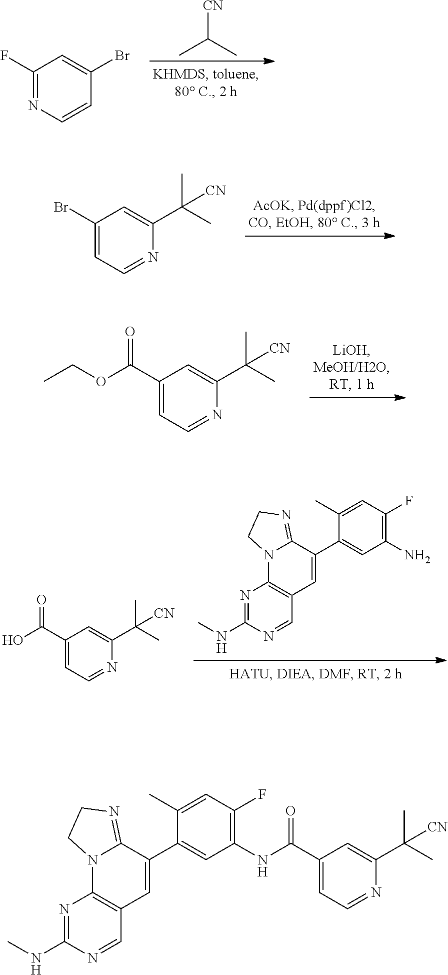

### Step 1: Synthesis of 2-(4-bromopyridin-2-yl)-2-methylpropanenitrile

To a solution of 4-bromo-2-fluoropyridine (500 mg, 2.84 mmol) in toluene (10 mL) was added isobutyronitrile (196 mg, 2.84 mmol) and KHMDS (1.0 M, 2.8 mL, 2.84 mmol). The reaction mixture was stirred for 2 h at 80° C., quenched with NH₄Cl (aq.) (20 mL) and extracted with EA (20 mL×2). The combined organic phase was washed with brine (30 mL), dried with Na₂SO₄, filtered and concentrated in vacuum. The residue was purified by column chromatography on silica gel (PE:EA=20:1) to afford 2-(4-bromopyridin-2-yl)-2-methylpropanenitrile (350 mg, 54.7% yield) as a white solid. ¹H NMR (400 MHz, DMSO-d6): δ 8.41 (d, J=4.2 Hz, 1H), 7.85 (d, J=1.2 Hz, 1H), 7.70-7.69 (m, 1H), 1.71 (s, 6H).

### Step 2: Synthesis of ethyl 2-(2-cyanopropan-2-yl)isonicotinate

To a solution of 2-(4-bromopyridin-2-yl)-2-methylpropanenitrile (350 mg, 1.56 mmol) in EtOH (6 mL) was added AcOK (457 mg, 4.67 mmol) and Pd(dppf)Cl₂ (114 mg, 0.16 mmol). The resulting mixture was stirred at 80° C. for 3 h under CO. The reaction mixture was diluted with water (10 mL) and extracted with EA (10 mL×3). The combined organic phase was washed with brine (10 mL), dried with Na₂SO₄, filtered and concentrated in vacuum. The residue was purified by column chromatography on silica gel (PE:EA=10:1) to afford ethyl 2-(2-cyanopropan-2-yl)isonicotinate (228 mg, 67.1% yield) as a red solid. LCMS (M+H⁺) m/z: 219.2.

### Step 3: Synthesis of 2-(2-cyanopropan-2-yl)isonicotinic acid

To a solution of ethyl 2-(2-cyanopropan-2-yl)isonicotinate (280 mg, 1.28 mmol) in MeOH/H₂O (5 mL/1 mL) was added LiOH (92 mg, 3.85 mmol), the reaction mixture was stirred for 1 h at RT. The reaction mixture was evaporated to remove MeOH, and extracted with EA. The aqueous phase was adjusted to pH=2-3 with 1N HCl, and extracted with DCM (20 mL×3). The combined organic phase was washed with brine (10 mL), dried with Na₂SO₄, filtered and concentrated in vacuum to afford 2-(2-cyanopropan-2-yl)isonicotinic acid (200 mg, 82% yield) as a white solid.

### Step 4: Synthesis of 2-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)isonicotinamide

A mixture of 6-(5-amino-4-fluoro-2-methylphenyl)-N-methyl-8,9-dihydroimidazo[1′,2′:1,6]pyrido[2,3-d]pyrimidin-2-amine (80 mg, 0.25 mmol), 2-(2-cyanopropan-2-yl)isonicotinic acid (50 mg, 0.26 mmol), HATU (140 mg, 0.37 mmol) and DIEA (96 mg, 0.74 mmol) in DMF (3 mL) was stirred at RT for 2 h. The reaction mixture was diluted with water (20 mL), filtered. The filter cake was purified by Prep-HPLC (0.1% NH3H2O) to afford 2-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)isonicotinamide (11.3 mg, 9.3% yield) as a yellow solid. ¹H NMR (400 MHz, DMSO-d₆): δ 10.46 (s, 1H), 8.80 (d, J=4.8 Hz, 1H), 8.23-8.17 (m, 1H), 8.04 (s, 1H), 7.87-7.85 (m, 2H), 7.23 (d, J=3.2 Hz, 1H), 7.13 (s, 1H), 4.02-4.01 (m, 2H), 3.93-3.88 (m, 2H), 2.84 (d, J=4.4 Hz, 3H), 2.24 (s, 3H), 1.76 (s, 6H). LCMS (M+H⁺) m/z: 497.3.

### Example 167: Preparation of 3-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)benzamide (Compound 167)

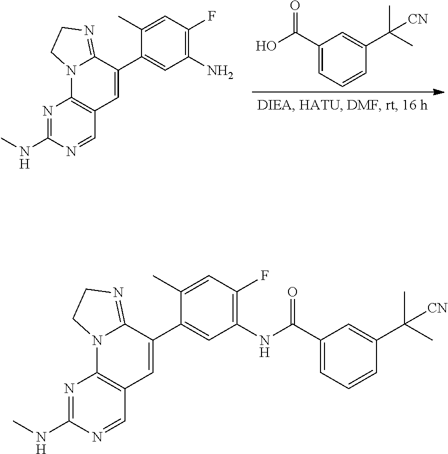 [SMILES: CNc1ncc2c(n1)N1CCN=C1C(c1cc(N)c(F)cc1C)=C2 ; CC(C)(C#N)c1cccc(C(=O)O)c1 ; CNc1ncc2c(n1)N1CCN=C1C(c1cc(NC(=O)c3cccc(C(C)(C)C#N)c3)c(F)cc1C)=C2]

The preparation of 6-(5-amino-4-fluoro-2-methylphenyl)-N-methyl-8,9-dihydroimidazo[1′,2′:1,6]pyrido[2,3-d]pyrimidin-2-amine was described in Example 139.

### Step 1: Synthesis of 3-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)benzamide

To a solution of 3-(2-cyanopropan-2-yl)benzoic acid (38 mg, 0.20 mmol), 6-(5-amino-4-fluoro-2-methylphenyl)-N-methyl-8,9-dihydroimidazo[1′,2′:1,6]pyrido[2,3-d]pyrimidin-2-amine (65 mg, 0.20 mmol), and HATU (115 mg, 0.30 mmol) in DMF (5.0 mL) was added DIEA (52 mg, 0.40 mmol). The resulting mixture was stirred at r.t. for 16 h under N₂. The reaction mixture was quenched with water (20 mL) and extracted with DCM (20 mL×3). The combined organic layers were washed with brine (20 mL), dried over Na₂SO₄, filtered, concentrated to give crude product, which was purified by Pre-HPLC (0.1% NH₃·H₂O) to afford 3-(2-cyanopropan-2-yl)-N-(2-fluoro-4-methyl-5-(2-(methylamino)-8,9-dihydroimidazo[1′,2′:1,6]pyrido[2,3-d]pyrimidin-6-yl)phenyl)benzamide (18.6 mg, 18.8% yield) as a yellow solid. ¹H NMR (400 MHz, DMSO-d₆): δ 10.19 (s, 1H), 8.24-8.22 (m, 1H), 8.08 (s, 1H), 7.94 (d, J=7.6 Hz, 1H), 7.77-7.75 (m, 1H), 7.61-7.57 (m, 1H), 7.45-7.40 (m, 2H), 7.22-7.13 (m, 2H), 4.12-3.99 (m, 2H), 3.91 (t, J=8.0 Hz, 2H), 2.84 (s, 3H), 2.24 (s, 3H), 1.75 (s, 6H). LCMS (M+H⁺) m/z: 496.2.

### Example 168: Preparation of 3-(2-cyanopropan-2-yl)-N-(5-(2-(dimethylamino)-8,9-dihydroimidazo[1′,2′:1,6]pyrido[2,3-d]pyrimidin-6-yl)-2-fluoro-4-methylphenyl)benzamide (Compound 168)

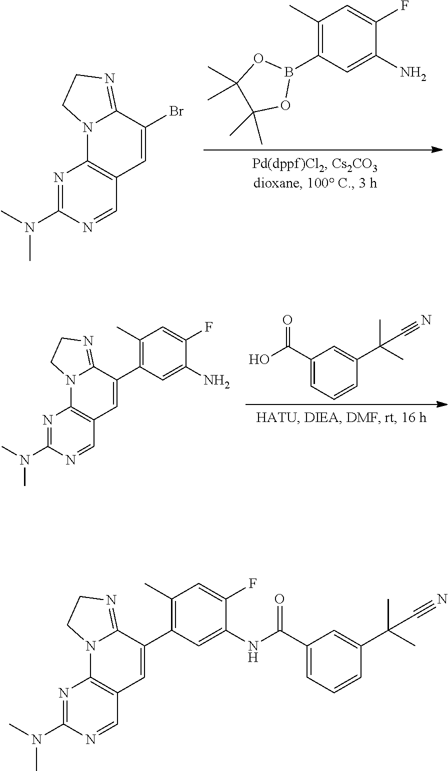

### Step 1: Synthesis of 6-(5-amino-4-fluoro-2-methylphenyl)-N,N-dimethyl-8,9-dihydroimidazo[1′,2′:1,6]pyrido[2,3-d]pyrimidin-2-amine

To a solution of 6-bromo-N,N-dimethyl-8,9-dihydroimidazo[1′,2′:1,6]pyrido[2,3-d]pyrimidin-2-amine (240 mg, 0.815 mmol), 2-fluoro-4-methyl-5-(4,4,5,5-tetramethyl-1,3,2-dioxaborolan-2-yl)aniline (307 mg, 1.223 mmol) and Cs₂CO₃ (796 mg, 2.445 mmol) in dioxane (12 mL) and H₂O (3 mL) was added Pd(dppf)Cl₂ (60 mg, 0.082 mmol). The reaction mixture was stirred at 100° C. under N₂ for 3 h. The result solution was concentrated and purified by flash chromatography to afford 6-(5-amino-4-fluoro-2-methylphenyl)-N,N-dimethyl-8,9-dihydroimidazo[1′,2′:1,6]pyrido[2,3-d]pyrimidin-2-amine (260 mg, 94.2% of yield) as a yellow solid.

### Step 2: Synthesis of 3-(2-cyanopropan-2-yl)-N-(5-(2-(dimethylamino)-8,9-dihydroimidazo[1′,2′:1,6]pyrido[2,3-d]pyrimidin-6-yl)-2-fluoro-4-methylphenyl)benzamide formate

To a solution of 2-fluoro-4-methyl-5-(4,4,5,5-tetramethyl-1,3,2-dioxaborolan-2-yl)aniline (100 mg, 0.295 mmol) and 3-(2-cyanopropan-2-yl)benzoic acid (56 mg, 0.295) in DMF (5 mL) was added DIEA (114 mg, 0.886 mmol) and HATU (337 mg, 0.886 mmol). The reaction mixture was stirred at R.T under N₂ for 3 hours. H₂O (20 mL) was added and the reaction mixture was extracted with EA twice. The combined extracts were washed with brine (20 mL). Concentration in vacuum and purification by prep-TLC (DCM:MeOH=30:1) and prep-HPLC (0.1%/FA/CH₃CN/H₂O) gave 3-(2-cyanopropan-2-yl)-N-(5-(2-(dimethylamino)-8,9-dihydroimidazo[1′,2′:1,6]pyrido[2,3-d]pyrimidin-6-yl)-2-fluoro-4-methylphenyl)benzamide formate (16.55 mg, 11% yield) as a yellow solid. ¹H NMR (400 MHz, DMSO-d₆) δ 10.20 (s, 1H), 8.30 (s, 1H), 8.25 (s, 1H), 8.09 (s, 1H), 7.94 (d, J=7.6 Hz, 1H), 7.75 (d, J=8.0 Hz, 1H), 7.59 (t, J=7.6 Hz, 1H), 7.40 (d, J=7.6 Hz, 1H), 7.20 (d, J=12.0 Hz, 1H), 7.14 (s, 1H), 4.05-4.01 (m, 2H), 3.94-3.89 (m, 2H), 2.24 (s, 3H), 1.75 (s, 6H). LCMS (M+H⁺) m/z: 510.2.

### Example 170: Preparation of 3-(2-hydroxypropan-2-yl)-N-(4-methyl-3-(2-(methylamino)-8,9-dihydroimidazo[1′,2′:1,6]pyrido[2,3-d]pyrimidin-6-yl)phenyl)benzamide (Compound 170)

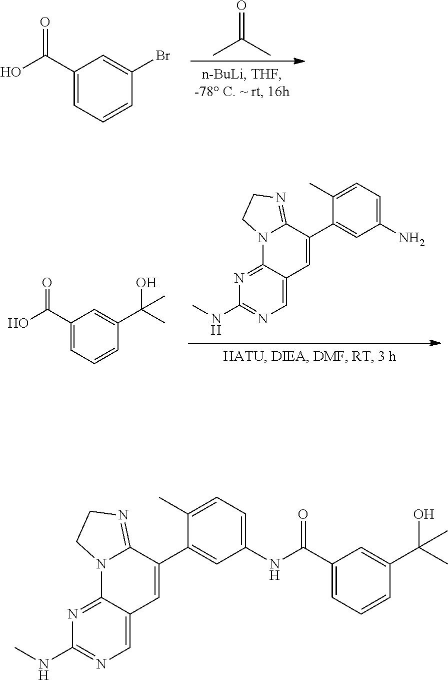

### Step 1: Synthesis of 3-(2-hydroxypropan-2-yl)benzoic acid

A 2.5 M solution of n-butyllithium (4 mL, 9.95 mmol) in a hexane fraction were added dropwise to a solution of 1.0 g (4.97 mmol) of 3-bromobenzoic acid in 20 mL of anhydrous THE under argon and at −78° C. After 20 min, 730 uL (9.95 mmol) of acetone were added dropwise at the same temperature. The reaction mixture was stirred at −78° C. for a further hour and then allowed to warm to RT over the course of about 1 h. The reaction mixture was then hydrolysed by careful addition of a few drops of saturated aqueous ammonium chloride solution and AcOH (2.0 mL). The mixture was diluted with 200 mL of water and extracted two times with about 50 mL of ethyl acetate. The combined organic extracts were washed with saturated aqueous sodium chloride solution, dried over magnesium sulphate, filtered and freed from the solvent on a rotary evaporator. The residue obtained was purified by flash (PE:EA=3:1). Evaporation and drying under high vacuum gave 3-(2-hydroxypropan-2-yl)benzoic acid (356 mg, 39% yield). ¹H NMR (400 MHz, CDCl3): 8.23 (s, 1H), 8.02-7.99 (m, 1H), 7.80-7.77 (m, 1H), 7.48-7.46 (m, 1H), 1.63 (s, 6H). LCMS (M-18)⁻ m/z: 163.0.

### Step 2: Synthesis of 3-(2-hydroxypropan-2-yl)-N-(4-methyl-3-(2-(methylamino)-8,9-dihydroimidazo[1′,2′:1,6]pyrido[2,3-d]pyrimidin-6-yl)phenyl)benzamide

The mixture of 3-(2-hydroxypropan-2-yl)benzoic acid (36 mg, 0.18 mmol), HATU (76 mg, 0.18 mmol), DIPEA (58 mg, 0.45 mmol), 6-(5-amino-2-methylphenyl)-N-methyl-8,9-dihydroimidazo[1′,2′:1,6]pyrido[2,3-d]pyrimidin-2-amine (45 mg, 0.15 mmol) in DMF (2.0 mL) was stirred at 10° C. for 2 h. Water was added, the reaction mixture was extracted with EA. The combined extracts were washed with brine. Concentration and purification by The reaction was purification by flash (DCM:MeOH=10:1) and then HPLC (0.5% FA) to give pure 3-(2-hydroxypropan-2-yl)-N-(4-methyl-3-(2-(methylamino)-8,9-dihydroimidazo[1′,2′:1,6]pyrido[2,3-d]pyrimidin-6-yl)phenyl)benzamide (14.1 mg, 20% yield) as yellow solid. ¹H NMR (400 MHz, DMSO-d6): δ 10.35 (s, 1H), 9.71 (s, 1H), 8.87 (s, 1H), 8.53-8.52 (m, 1H), 8.13 (s, 1H), 8.02 (s, 1H), 7.84-7.66 (m, 3H), 7.46 (t, J=7.6 Hz, 1H), 7.37 (d, J=8.8 Hz, 1H), 4.67-4.57 (m, 2H), 4.04-3.99 (m, 2H), 2.97-2.95 (d, 3H), 2.18 (s, 3H), 1.47-1.43 (s, 6H). LCMS (M+H+) m/z: 469.0.

### Example 171: Preparation of N-(2-fluoro-4-methyl-5-(2-(methylamino)-8,9-dihydroimidazo[1′,2′:1,6]pyrido[2,3-d]pyrimidin-6-yl)phenyl)-3-(2-hydroxypropan-2-yl)benzamide (Compound 171)

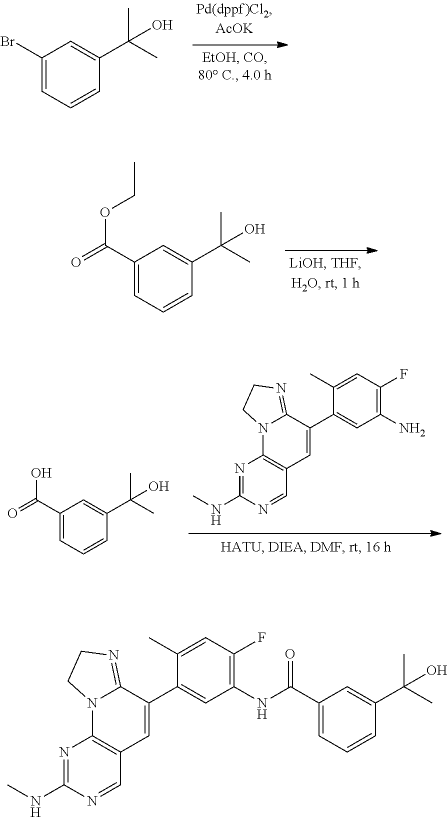

### Step 1: Synthesis of ethyl 3-(2-hydroxypropan-2-yl)benzoate

A mixture of A mixture of 1-((6-chloro-4-(trifluoromethyl)pyridin-3-yl)methyl)-4-ethylpiperazine (100 mg, 0.47 mmol), AcOK (182 mg, 1.86 mmol) and Pd(dppf)Cl₂ (17 mg, 0.02 mmol) in EtOH (20 mL) was degassed, charged with CO for three times and stirred at 80° C. for 16 h under CO. The reaction mixture was concentrated and the residue was purified by column chromatography on silica gel (PE:EA=4:1) to afford ethyl 3-(2-hydroxypropan-2-yl)benzoate (80 mg, 78% yield) as a white solid. LCMS (M-17) m/z: 191.1.

### Step 2: Synthesis of 3-(2-hydroxypropan-2-yl)benzoic acid

LiOH (69 mg, 2.88 mmol) was added to the mixture of ethyl ethyl 3-(2-hydroxypropan-2-yl)benzoate (100 mg, 0.29 mmol) in THF:H₂O (5:1) (5 mL). The mixture was stirred at r.t for 2 hours. The reaction mixture was quenched with water (20 mL) and extracted with DCM (20 mL). The aqueous phase was adjusted to pH=3 with 2N HCl and then concentrated to afford 3-(2-hydroxypropan-2-yl)benzoic acid (80 mg, crude) as a white solid. LCMS (M−H)⁻ m/z: 179.1.

### Step 3: Synthesis of 5-((4-ethylpiperazin-1-yl)methyl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide

A mixture of 3-(2-hydroxypropan-2-yl)benzoic acid (50 mg, 0.28 mmol), 6-(5-amino-4-fluoro-2-methylphenyl)-N-methyl-8,9-dihydroimidazo[1′,2′:1,6]pyrido[2,3-d]pyrimidin-2-amine (90 mg, 0.28 mmol), DIEA (25 mg, 0.20 mmol) in DMF (2 mL) was added HATU (211 mg, 0.56 mmol). The mixture was stirred at r.t for 16 hours. The reaction mixture was quenched with water (30 mL) and extracted with DCM (30 mL×3). The combined organic layers were washed with brine (60 mL), dried over Na2SO4, filtered, concentrated to afford crude, which was purified by Prep-HPLC (0.1% NH3·H2O) to afford 5-((4-ethylpiperazin-1-yl)methyl)-N-(4-methyl-3-(2-(methylamino)-8,9-dihydroimidazo[1′,2′:1,6]pyrido[2,3-d]pyrimidin-6-yl)phenyl)-4-(trifluoromethyl)picolinamide (14.8 mg, 10.9% yield) as a yellow solid. ¹H NMR (400 MHz, DMSO-d₆): δ 10.06 (s, 1H), 8.24-8.19 (m, 1H), 8.07 (s, 1H), 7.81 (d, J=8.0 Hz, 1H), 7.70 (d, J=7.6 Hz, 1H), 7.47-7.38 (m, 3H), 7.20-7.14 (m, 2H), 5.15 (s, 1H), 4.04-3.96 (m, 2H), 3.91 (t, J=8.0 Hz, 2H), 2.85 (m, 3H), 2.20 (s, 3H), 1.47 (s, 6H). LCMS (M+H⁺) m/z: 487.2

### Example 172: Preparation of 6-(2,4-dichlorophenyl)-N-(3-fluoro-4-(4-methylpiperazin-1-yl)phenyl)-8,9-dihydroimidazo[1′,2′:1,6]pyrido[2,3-d]pyrimidin-2-amine (Compound 172)

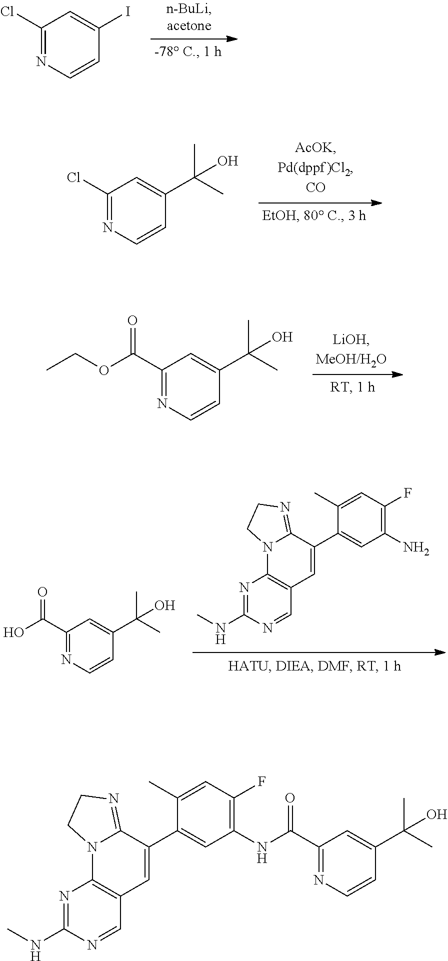

### Step 1: Synthesis of 2-(2-chloropyridin-4-yl)propan-2-ol

To a solution of 2-chloro-4-iodopyridine (1.0 g, 4.18 mmol) in THF (15 mL) was added n-BuLi (1.6 M, 2.6 mL, 4.18 mmol) at −78° C. After 15 min, acetone (728 mg, 12.55 mmol) was added. The reaction mixture was stirred for 2 h at −78° C., quenched with NH₄Cl (aq.) (20 mL) and extracted with EA (20 mL×2). The combined organic phase was washed with brine (20 mL), dried with Na₂SO₄, filtered and concentrated in vacuum. The residue was purified by column chromatography on silica gel (PE:EA=5:1) to afford 2-(2-chloropyridin-4-yl)propan-2-ol (490 mg, 68.5% yield) as a yellow oil. LCMS (M+H⁺) m/z: 172.1.

### Step 2: Synthesis of ethyl 4-(2-hydroxypropan-2-yl)picolinate

To a solution of 2-(2-chloropyridin-4-yl)propan-2-ol (49 mg, 2.87 mmol) in EtOH (10 mL) was added AcOK (842 mg, 8.60 mmol) and Pd(dppf)Cl₂ (210 mg, 0.29 mmol). The resulting mixture was stirred at 80° C. for 3 h under CO. The reaction mixture was diluted with water (10 mL) and extracted with EA (10 mL×3). The combined organic phase was washed with brine (10 mL), dried with Na₂SO₄, filtered and concentrated in vacuum. The residue was purified by column chromatography on silica gel (PE:EA=2:1) to afford ethyl 4-(2-hydroxypropan-2-yl)picolinate (400 mg, 63% yield) as a red oil. LCMS (M+H⁺) m/z: 210.2.

### Step 3: Synthesis of 4-(2-hydroxypropan-2-yl)picolinic acid

To a solution of ethyl 4-(2-hydroxypropan-2-yl)picolinate (400 mg, 1.91 mmol) in MeOH/H₂O (10 mL/2 mL) was added LiOH (138 mg, 5.74 mmol). The reaction mixture was stirred for 1 h at RT. The reaction mixture was evaporated to remove MeOH and extracted with EA. The aqueous phase was adjusted to pH=2-3 with 1N HCl, and concentrated in vacuum to afford 4-(2-hydroxypropan-2-yl)picolinic acid (1.0 g, crude) as a yellow oil. LCMS (M+H⁺) m/z: 182.1.

### Step 4: Synthesis of N-(2-fluoro-4-methyl-5-(2-(methylamino)-8,9-dihydroimidazo[1′,2′:1,6]pyrido[2,3-d]pyrimidin-6-yl)phenyl)-4-(2-hydroxypropan-2-yl)picolinamide

A mixture of 6-(5-amino-4-fluoro-2-methylphenyl)-N-methyl-8,9-dihydroimidazo[1′,2′:1,6]pyrido[2,3-d]pyrimidin-2-amine (80 mg, 0.25 mmol), 4-(2-hydroxypropan-2-yl)picolinic acid (268 mg, 0.19 mmol), HATU (141 mg, 0.37 mmol) and DIEA (95 mg, 0.74 mmol) in DMF (5 mL) at RT was stirred at r.t. for 2 h. The reaction mixture was diluted with water (10 mL), extracted with EA (20 mL×3). The combined organic phase was washed with brine (20 mL×3), dried with Na₂SO₄, filtered and concentrated. The residue was purified by Pre-HPLC (0.1% NH₃H₂O) to afford N-(2-fluoro-4-methyl-5-(2-(methylamino)-8,9-dihydroimidazo[1′,2′:1,6]pyrido[2,3-d]pyrimidin-6-yl)phenyl)-4-(2-hydroxypropan-2-yl)picolinamide (36 mg, 30% yield) as a yellow solid. ¹H NMR (400 MHz, DMSO-d₆): δ 10.35 (s, 1H), 8.67 (d, J=4.8 Hz, 1H), 8.25-8.19 (m, 2H), 7.96 (d, J=8.0 Hz, 1H), 7.75 (dd, J=4.4, 1.6 Hz, 1H), 7.42-7.41 (m, 1H), 7.25 (d, J=11.6 Hz, 1H), 7.14 (s, 1H), 5.47 (s, 1H), 4.05-3.99 (m, 2H), 3.93-3.88 (m, 2H), 2.85 (d, J=4.0 Hz, 3H), 2.22 (s, 3H), 1.46 (s, 6H). LCMS (M+H⁺) m/z: 488.3.

### Example 173: Preparation of 3-(2-cyanopropan-2-yl)-N-(2-fluoro-4-methyl-3-(2-(methylamino)-8,9-dihydroimidazo[1′,2′:1,6]pyrido[2,3-d]pyrimidin-6-yl)phenyl)benzamide (Compound 173)

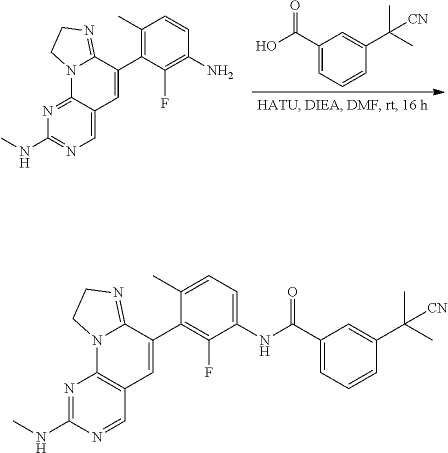 [SMILES: CNc1ncc2c(n1)N1CCN=C1C(c1c(C)ccc(N)c1F)=C2 ; CC(C)(C#N)c1cccc(C(=O)O)c1 ; CNc1ncc2c(n1)N1CCN=C1C(c1c(C)ccc(NC(=O)c3cccc(C(C)(C)C#N)c3)c1F)=C2]

The preparation of 6-(3-amino-2-fluoro-6-methylphenyl)-N-methyl-8,9-dihydroimidazo[1′,2′:1,6]pyrido[2,3-d]pyrimidin-2-amine was described in Example 141.

### Step 1: Synthesis of 3-(2-cyanopropan-2-yl)-N-(2-fluoro-4-methyl-3-(2-(methylamino)-8, 9-dihydroimidazo[1′, 2′:1, 6]pyrido[2, 3-d]pyrimidin-6-yl)phenyl) benzamide

To a mixture of 6-(3-amino-2-fluoro-6-methylphenyl)-N-methyl-8,9-dihydroimidazo[1′,2′:1,6]pyrido[2,3-d]pyrimidin-2-amine (50 mg, 0.15 mmol, 1.0 eq) and 3-(2-cyanopropan-2-yl)benzoic acid (43 mg, 0.23 mmol, and 1.5 eq) in DMF (3 mL) was added DIEA (0.1 mL) and HATU (88 mg, 0.23 mmol, 1.5 eq). The mixture was stirred at 20° C. overnight. LCMS showed the reaction was OK. Water was added, the reaction mixture was extracted with EA. The combined extracts were washed with brine. Concentration and purification by prep-HPLC (0.1% NH₄OH) to afford 3-(2-cyanopropan-2-yl)-N-(2-fluoro-4-methyl-3-(2-(methylamino)-8, 9-dihydroimidazo[1′, 2′:1, 6]pyrido[2, 3-d]pyrimidin-6-yl) phenyl) benzamide (4.8 mg, 6.5% yield) as a yellow solid. ¹H NMR (400 MHz, CD₃OD): δ 8.57-8.51 (m, 2H), 8.11 (s, 1H), 7.92 (d, J=8.0 Hz, 1H), 7.79 (d, J=7.6 Hz, 1H), 7.67 (t, J=8.0 Hz, 1H), 7.59 (t, J=8.0 Hz, 1H), 7.23 (d, J=7.2 Hz, 1H), 4.59-4.48 (m, 2H), 4.11-4.05 (m, 2H), 3.04 (s, 3H), 2.29 (s, 3H), 1.78 (s, 6H). LCMS (M+H⁺) m/z: 496.1.

### Example 174: Preparation of 4-(2-cyanopropan-2-yl)-N-(2-fluoro-4-methyl-3-(2-(methylamino)-8,9-dihydroimidazo[1′,2′:1,6]pyrido[2,3-d]pyrimidin-6-yl)phenyl)picolinamide (Compound 174)

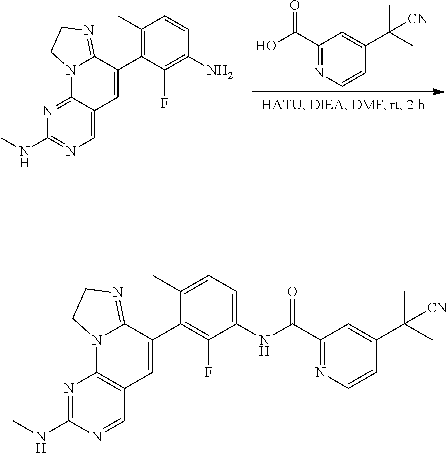 [SMILES: CNc1ncc2c(n1)N1CCN=C1C(c1c(C)ccc(N)c1F)=C2 ; CC(C)(C#N)c1ccnc(C(=O)O)c1 ; CNc1ncc2c(n1)N1CCN=C1C(c1c(C)ccc(NC(=O)c3cc(C(C)(C)C#N)ccn3)c1F)=C2]

### Step 1: Synthesis of 4-(2-cyanopropan-2-yl)-N-(2-fluoro-4-methyl-3-(2-(methylamino)-8,9-dihydroimidazo[1′,2′:1,6]pyrido[2,3-d]pyrimidin-6-yl)phenyl)picolinamide

To a mixture of 6-(3-amino-2-fluoro-6-methylphenyl)-N-methyl-8,9-dihydroimidazo[1′,2′:1,6]pyrido[2,3-d]pyrimidin-2-amine (50 mg, 0.15 mmol, 1.0 eq) and 4-(2-cyanopropan-2-yl)picolinic acid (45 mg, 0.23 mmol, and 1.5 eq) in DMF (3 mL) was added DIEA (0.1 mL) and HATU (88 mg, 0.23 mmol, 1.5 eq). The mixture was stirred at 20° C. overnight. LCMS showed the reaction was OK. Water was added, the reaction mixture was extracted with EA. The combined extracts were washed with brine. Concentration and purification by prep-HPLC (0.1% HCl) to afford 4-(2-cyanopropan-2-yl)-N-(2-fluoro-4-methyl-3-(2-(methylamino)-8,9-dihydroimidazo[1′,2′:1,6]pyrido[2,3-d]pyrimidin-6-yl)phenyl)picolinamide (8.6 mg, 11.6% yield) as a yellow solid. ¹H NMR (400 MHz, CD₃OD): δ 8.86 (s, 1H), 8.75 (d, J=4.8 Hz, 1H), 8.41 (s, 1H), 8.24 (t, J=8.0 Hz, 1H), 8.17 (s, 1H), 7.84-7.82 (m, 1H), 7.31 (d, J=8.4 Hz, 1H), 4.86-4.79 (m, 2H), 4.20-4.15 (m, 2H), 3.10 (s, 3H), 2.30 (s, 3H), 1.81 (s, 6H). LCMS (M+H⁺) m/z: 497.1.

### Example 175: Preparation of isopropyl (6-(5-(4-(2-cyanopropan-2-yl)picolinamido)-4-fluoro-2-methylphenyl)-8,9-dihydroimidazo[1′,2′:1,6]pyrido[2,3-d]pyrimidin-2-yl)carbamate (Compound 175)

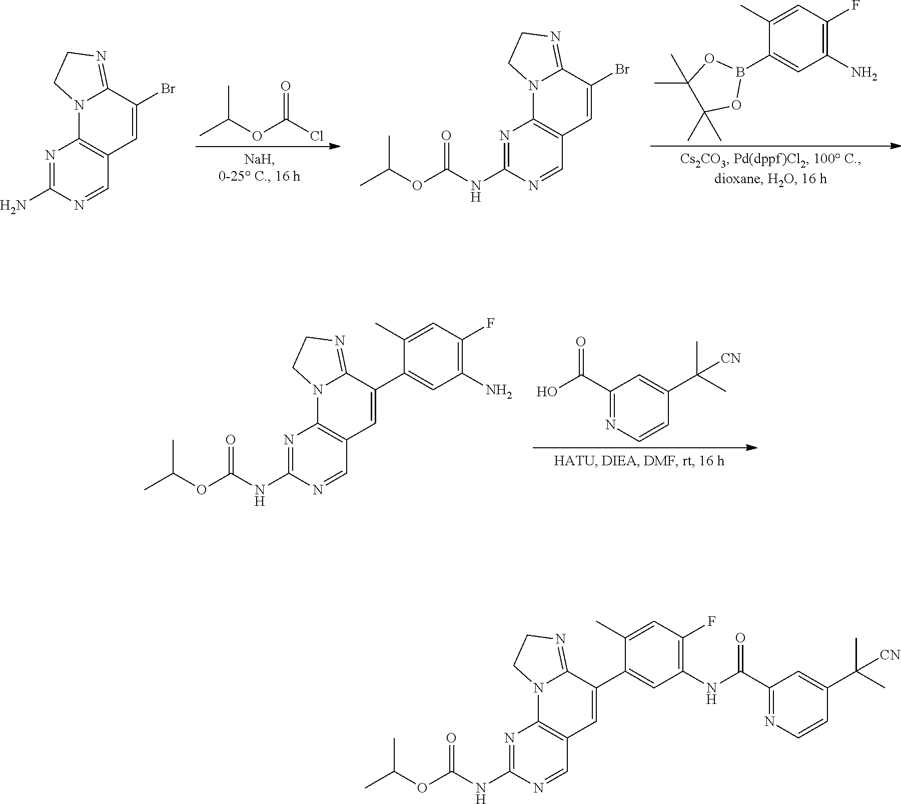

### Step 1: Synthesis of isopropyl (6-bromo-8,9-dihydroimidazo[1′,2′:1,6]pyrido[2,3-d]pyrimidin-2-yl)carbamate

To a solution of 6-bromo-8,9-dihydroimidazo[1′,2′:1,6]pyrido[2,3-d]pyrimidin-2-amine (300 mg, 1.28 mmol) in THF (15.0 mL) was added NaH (90 mg, 2.26 mmol) at 0° C., the mixture was stirred for 1 h and then isopropyl carbonochloridate (379 mg, 3.38 mmol) was added. The result mixture was stirred at rt for 16 h under N₂. The reaction mixture was concentrated in vacuum and purified by column chromatography on silica gel (DCM/MeOH=10/1) to afford of isopropyl (6-bromo-8,9-dihydroimidazo[1′,2′:1,6]pyrido[2,3-d]pyrimidin-2-yl)carbamate (90 mg, 20% yield) as brown solid. LCMS (M+H⁺) m/z: 352.2 and 354.2.

### Step 2: Synthesis of isopropyl (6-(5-amino-4-fluoro-2-methylphenyl)-8,9-dihydroimidazo[1′,2′:1,6]pyrido[2,3-d]pyrimidin-2-yl)carbamate

A mixture of isopropyl (6-bromo-8,9-dihydroimidazo[1′,2′:1,6]pyrido[2,3-d]pyrimidin-2-yl)carbamate (25 mg, 0.071 mmol), 2-fluoro-4-methyl-5-(4,4,5,5-tetramethyl-1,3,2-dioxaborolan-2-yl)aniline (22 mg, 0.085 mmol), Cs₂CO₃ (69 mg, 0.213 mmol), and Pd(dppf)Cl₂ (5 mg, 0.007 mmol) in dioxane (5 mL) and H₂O (0.5 mL) was degassed, charged with N₂ three times and stirred at 90° C. for 16 h under N₂. The reaction mixture was concentrated in vacuum and purified by column chromatography on silica gel (DCM/MeOH=15/1) to give isopropyl (6-(5-amino-4-fluoro-2-methylphenyl)-8,9-dihydroimidazo[1′,2′:1,6]pyrido[2,3-d]pyrimidin-2-yl)carbamate (23 mg, 81.8% yield) as brown solid. LCMS (M+H⁺) m/z: 397.3.

### Step 3: Synthesis of isopropyl (6-(5-(4-(2-cyanopropan-2-yl)picolinamido)-4-fluoro-2-methylphenyl)-8,9-dihydroimidazo[1′,2′:1,6]pyrido[2,3-d]pyrimidin-2-yl)carbamate formic acid salt

To a solution of 4-(2-cyanopropan-2-yl)picolinic acid (14 mg, 0.07 mmol), isopropyl (6-(5-amino-4-fluoro-2-methylphenyl)-8,9-dihydroimidazo[1′,2′:1,6]pyrido[2,3-d]pyrimidin-2-yl)carbamate (23 mg, 0.065 mmol), and HATU (50 mg, 0.13 mmol) in DMF (3.0 mL) was added DIEA (25 mg, 0.196 mmol). The resulting mixture was stirred at r.t. for 16 h under N₂. The reaction mixture was quenched with water (20 mL) and extracted with DCM (20 mL×3), The combined organic layers were washed with brine (20 mL), dried over Na₂SO4, filtered, concentrated to afford crude, which was purified by Prep-HPLC (0.1% FA) to afford isopropyl (6-(5-(4-(2-cyanopropan-2-yl)picolinamido)-4-fluoro-2-methylphenyl)-8,9-dihydroimidazo[1′,2′:1,6]pyrido[2,3-d]pyrimidin-2-yl)carbamate formic acid salt (6.4 mg, 16.1% yield) as a yellow solid. ¹H NMR (400 MHz, DMSO-d₆): δ 10.42 (s, 1H), 10.25 (s, 1H), 8.82 (d, J=5.2 Hz, 1H), 8.43 (s, 1H), 8.26 (s, 1H), 8.25 (s, 1H), 8.22 (s, 1H), 7.92 (d, J=8.4 Hz, 1H), 7.87 (d, J=6.0, 2.0 Hz, 1H), 7.28 (t, J=4.4 Hz, 2H), 4.94-4.87 (m, 1H), 4.06 (t, J=8.4 Hz, 2H), 3.95 (t, J=8.4 Hz, 2H), 2.25 (s, 3H), 1.76 (s, 6H), 1.27 (s, 3H), 1.25 (s, 3H). LCMS (M+H⁺) m/z: 569.6.

### Example 176: Preparation of isopropyl (6-(4-fluoro-2-methyl-5-(4-(trifluoromethyl)picolinamido)phenyl)-8,9-dihydroimidazo[1′,2′:1,6]pyrido[2,3-d]pyrimidin-2-yl)carbamate (Compound 176)

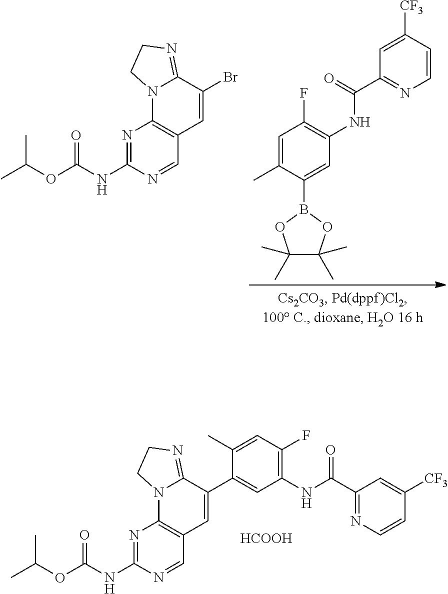

### Step 1: Synthesis of isopropyl (6-(4-fluoro-2-methyl-5-(4-(trifluoromethyl)picolinamido)phenyl)-8,9-dihydroimidazo[1′,2′:1,6]pyrido[2,3-d]pyrimidin-2-yl)carbamate formic acid salt

A mixture of isopropyl (6-bromo-8,9-dihydroimidazo[1′,2′:1,6]pyrido[2,3-d]pyrimidin-2-yl)carbamate (30 mg, 0.085 mmol), N-(2-fluoro-4-methyl-5-(4,4,5,5-tetramethyl-1,3,2-dioxaborolan-2-yl)phenyl)-4-(trifluoromethyl)picolinamide (43 mg, 0.102 mmol), Cs₂CO₃ (83 mg, 0.256 mmol), and Pd(dppf)Cl₂ (6 mg, 0.008 mmol) in dioxane (5 mL) and H₂O (0.5 mL) was degassed and charged with N₂ three times and stirred at 90° C. for 16 h under N₂. The reaction mixture was concentrated in vacuum and purified by column chromatography on silica gel (DCM/MeOH=10/1) to give crude product, which was purified by Prep-HPLC (0.1% FA) to afford isopropyl (6-(4-fluoro-2-methyl-5-(4-(trifluoromethyl)picolinamido)phenyl)-8,9-dihydroimidazo[1′,2′:1,6]pyrido[2,3-d]pyrimidin-2-yl)carbamate formic acid salt (10.8 mg, 20.7% yield) as a yellow solid. ¹H NMR (400 MHz, DMSO-d₆): δ 10.48 (s, 1H), 10.24 (s, 1H), 9.06 (d, J=5.2 Hz, 1H), 8.42 (s, 1H), 8.33 (s, 1H), 8.27 (s, 1H), 8.14 (d, J=5.2 Hz, 1H), 7.87 (d, J=8.0 Hz, 1H), 7.28 (t, J=6.4 Hz, 2H), 4.94-4.87 (m, 1H), 4.06 (t, J=8.4 Hz, 2H), 3.95 (t, J=8.4 Hz, 2H), 2.26 (s, 3H), 1.27 (s, 3H), 1.25 (s, 3H). LCMS (M+H⁺) m/z: 570.5.

### Example 177: Preparation of N-(2-fluoro-4-methyl-5-(2-(oxetan-3-ylamino)-8,9-dihydroimidazo[1′,2′:1,6]pyrido[2,3-d]pyrimidin-6-yl)phenyl)-3-(trifluoromethyl)benzamide (Compound 177)

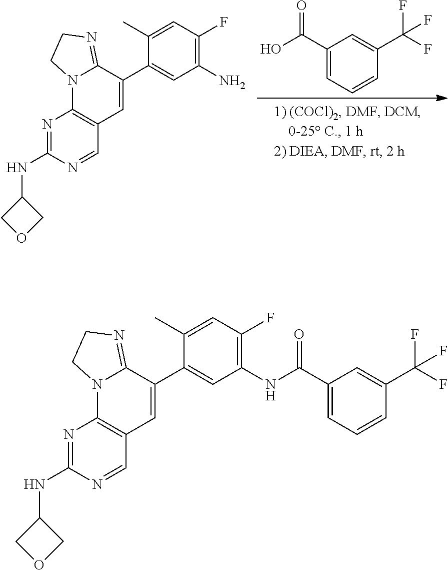 [SMILES: Cc1cc(F)c(N)cc1C1=Cc2cnc(NC3COC3)nc2N2CCN=C12 ; O=C(O)c1cccc(C(F)(F)F)c1 ; Cc1cc(F)c(NC(=O)c2cccc(C(F)(F)F)c2)cc1C1=Cc2cnc(NC3COC3)nc2N2CCN=C12]

### Step 1: Synthesis of 3-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

To a solution of 3-(trifluoromethyl)benzoic acid (60 mg, 0.316 mmol) and DMF (1.2 uL, 0.016 mmol) in dry DCM (6 mL) was added oxalyl chloride (48 mg, 0.379 mmol) drop-wised at 0° C. under N₂, the mixture was stirred at r.t. for 1 h. The reaction mixture was concentrated and dissolved in dry DMF (3 mL), then a solution of DIEA (86 mg, 0.670 mmol) and 6-(5-amino-4-fluoro-2-methylphenyl)-N-(oxetan-3-yl)-8,9-dihydroimidazo[1′,2′:1,6]pyrido[2,3-d]pyrimidin-2-amine (123 mg, 0.335 mmol) in DMF (1 mL) was added. The mixture was stirred at r.t. for 2 h. The reaction mixture was diluted with H₂O (20 mL) and extracted with DCM (30 mL×3), dried over Na2SO4, filtered. The combined organic layers was concentrated in vacuum and purified by column chromatography on silica gel (DCM/MeOH=15/1) to give crude product, which was purified by Prep-HPLC (0.1% NH₃—H₂O) to afford 3-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (27.7 mg, 16.3% yield) as yellow solid. ¹H NMR (400 MHz, DMSO-d₆): δ 10.37 (s, 1H), 8.31 (s, 1H), 8.26 (d, J=8.0 Hz, 1H), 8.22-8.21 (m, 2H), 7.98 (d, J=7.6 Hz, 1H), 7.79 (t, J=7.6 Hz, 1H), 7.42 (d, J=8.0 Hz, 1H), 7.21 (d, J=11.6 Hz, 1H), 7.14 (s, 1H), 4.95-4.92 (m, 1H), 4.76 (t, J=6.4 Hz, 2H), 4.54 (t, J=6.4 Hz, 2H), 4.03-3.88 (m, 4H), 2.23 (s, 3H). LCMS (M+H⁺) m/z: 539.2.

### Example 178: Preparation of N-(2-fluoro-4-methyl-5-(2-(oxetan-3-ylamino)-8,9-dihydroimidazo[1′,2′:1,6]pyrido[2,3-d]pyrimidin-6-yl)phenyl)-4-(trifluoromethyl)picolinamide (Compound 178)

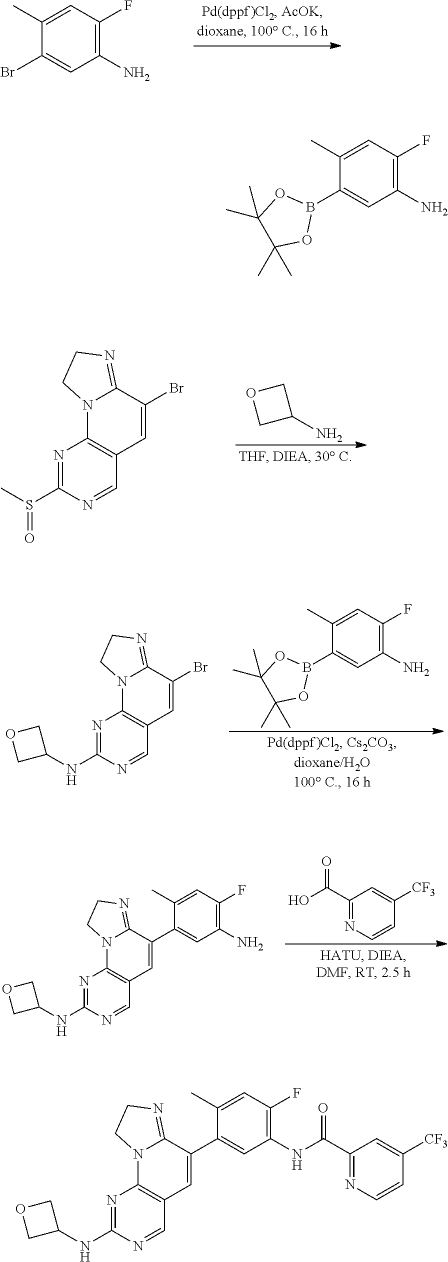

The preparation of 6-bromo-2-(methylsulfinyl)-8,9-dihydroimidazo[1′,2′:1,6]pyrido[2,3-d]pyrimidine was described in Example 26.

### Step 1: Synthesis of 2-fluoro-4-methyl-5-(4,4,5,5-tetramethyl-1,3,2-dioxaborolan-2-yl)aniline

A mixture of 5-bromo-2-fluoro-4-methylaniline (6.0 g, 29.4 mmol), Bis(pinacolato)diboron (8.96 g, 35.3 mmol), AcOK (8.6 g, 88.2 mmol) and [1,1′-Bis(diphenylphosphino)ferrocene]dichloropalladium(II) (1.07 g, 1.47 mmol) in dioxane (200 mL). The resulting mixture was degassed and charge with N₂ three times, stirred at 100° C. for 16 h. The reaction mixture was filtered and concentrated. The residue was purified by column chromatography on silica gel (PE:EA=5:1) to afford 2-fluoro-4-methyl-5-(4,4,5,5-tetramethyl-1,3,2-dioxaborolan-2-yl)aniline (7.1 g, 96% yield) as an off-white solid. LCMS (M+H⁺) m/z: 252.0.

### Step 2: Synthesis of 6-bromo-N-(oxetan-3-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (1.9 g, 6.07 mmol), oxetan-3-amine (2.2 g, 30.35 mmol), DIEA (1.57 g, 12.14 mmol) in THF (40.0 mL). The resulting mixture was stirred at 30° C. for 16 h. The reaction mixture was concentrated. The residue was triturated with EA (10.0 mL) to afford crude 6-bromo-N-(oxetan-3-yl)-8,9-dihydroimidazo[1′,2′:1,6]pyrido[2,3-d]pyrimidin-2-amine (2.0 g, 100% yield) as a yellow solid, which was used for the next step without purification. LCMS (M+H⁺) m/z: 321.9 and 323.9.

### Step 3: Synthesis of 6-(5-amino-4-fluoro-2-methylphenyl)-N-(oxetan-3-yl)-8,9-dihydroimidazo[1′,2′:1,6]pyrido[2,3-d]pyrimidin-2-amine

A mixture of 6-bromo-N-(oxetan-3-yl)-8,9-dihydroimidazo[1′,2′:1,6]pyrido[2,3-d]pyrimidin-2-amine (1.8 g, 5.59 mmol), 2-fluoro-4-methyl-5-(4,4,5,5-tetramethyl-1,3,2-dioxaborolan-2-yl)aniline (1.47 g, 5.87 mmol), Cs₂CO₃ (3.64 g, 11.18 mmol) and [1,1′-Bis(diphenylphosphino)ferrocene]dichloropalladium(II) (204 mg, 0.28 mmol) in dioxane (40.0 mL) and H₂O (4.0 mL) was stirred at 100° C. for 16 h under N₂. The reaction mixture was filtered and concentrated. The residue was purified by column chromatography on silica gel (DCM:MeOH=20:1) and then triturated by PE/EA (1:1, 10.0 mL) to afford 6-(5-amino-4-fluoro-2-methylphenyl)-N-(oxetan-3-yl)-8,9-dihydroimidazo[1′,2′:1,6]pyrido[2,3-d]pyrimidin-2-amine (1.21 g, 59% yield) as a yellow solid. LCMS (M+H⁺) m/z: 367.1.

### Step 4: Synthesis of N-(2-fluoro-4-methyl-5-(2-(oxetan-3-ylamino)-8,9-dihydroimidazo[1′,2′:1,6]pyrido[2,3-d]pyrimidin-6-yl)phenyl)-4-(trifluoromethyl)picolinamide

To a mixture of 4-(trifluoromethyl)picolinic acid (365 mg, 1.91 mmol), 6-(5-amino-4-fluoro-2-methylphenyl)-N-(oxetan-3-yl)-8,9-dihydroimidazo[1′,2′:1,6]pyrido[2,3-d]pyrimidin-2-amine (700 mg, 1.91 mmol) and HATU (1.45 g, 3.82 mmol) in DMF (12.0 mL) was added DIEA (493 mg, 3.82 mmol). The resulting mixture was stirred at r.t. for 2.5 h under N₂. The reaction mixture was added into water (45 mL) slowly, stirred at r.t. for 30 min, filtered. The collected cake was purified by silica column chromatography (DCM:MeOH=15:1, +0.2% NH₃-MeOH) to afford N-(2-fluoro-4-methyl-5-(2-(oxetan-3-ylamino)-8,9-dihydroimidazo[1′,2′:1,6]pyrido[2,3-d]pyrimidin-6-yl)phenyl)-4-(trifluoromethyl)picolinamide (606.4 mg, 52% yield) as a yellow solid. ¹H NMR (400 MHz, CD₃OD): δ 8.97 (d, J=5.2 Hz, 1H), 8.42 (s, 1H), 8.36 (s, 1H), 8.18 (d, J=8.0 Hz, 1H), 7.95 (d, J=4.8 Hz, 1H), 7.41 (s, 1H), 7.20 (d, J=11.6 Hz, 1H), 5.13 (t, J=6.8 Hz, 1H), 4.96 (t, J=6.8 Hz, 2H), 4.71-4.70 (m, 2H), 4.31 (t, J=9.6 Hz, 2H), 4.03 (t, J=10.0 Hz, 2H), 2.26 (s, 3H). LCMS (M+H⁺) m/z: 540.6.

### Example 179: Preparation of N-(2-fluoro-4-methyl-5-(2-(oxetan-3-ylamino)-8,9-dihydroimidazo[1′,2′:1,6]pyrido[2,3-d]pyrimidin-6-yl)phenyl)-6-(trifluoromethyl)picolinamide (Compound 179)

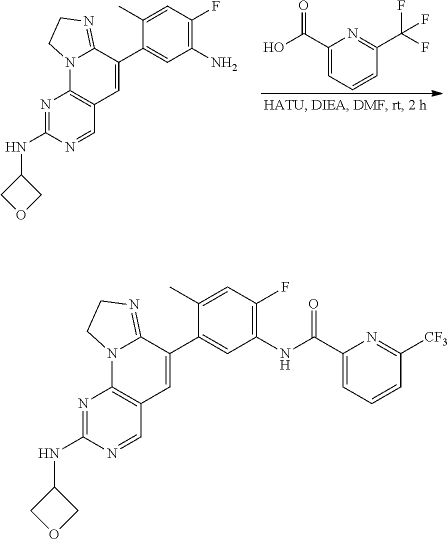 [SMILES: Cc1cc(F)c(N)cc1C1=Cc2cnc(NC3COC3)nc2N2CCN=C12 ; O=C(O)c1cccc(C(F)(F)F)n1 ; Cc1cc(F)c(NC(=O)c2cccc(C(F)(F)F)n2)cc1C1=Cc2cnc(NC3COC3)nc2N2CCN=C12]

### Step 1: Synthesis of N-(2-fluoro-4-methyl-5-(2-(oxetan-3-ylamino)-8,9-dihydroimidazo[1′,2′:1,6]pyrido[2,3-d]pyrimidin-6-yl)phenyl)-6-(trifluoromethyl)picolinamide

To the mixture of 6-(5-amino-4-fluoro-2-methylphenyl)-N-(oxetan-3-yl)-8,9-dihydroimidazo[1′,2′:1,6]pyrido[2,3-d]pyrimidin-2-amine (50 mg, 0.14 mmol), 6-(trifluoromethyl)picolinic acid (31 mg, 0.16 mmol), DIEA (54.2 mg, 0.42 mmol) in DMF (2 mL) was added HATU (77.8 mg, 0.20 mmol). The mixture was stirred at r.t for 2 hours. The reaction mixture was diluted with water (50 mL), filtered. The solid was washed with water (30 mL) and purified by column chromatography on silica gel (DCM:MeOH=10:1) to give the crude which was triturated with MeOH (20 mL) to afford N-(2-fluoro-4-methyl-5-(2-(oxetan-3-ylamino)-8,9-dihydroimidazo[1′,2′:1,6]pyrido[2,3-d]pyrimidin-6-yl)phenyl)-6-(trifluoromethyl)picolinamide (20.2 mg, 27.4% yield) as a yellow solid. ¹H NMR (400 MHz, DMSO-d₆): δ 10.23 (s, 1H), 8.41-8.36 (m, 2H), 8.25-8.19 (m, 4H), 7.84 (d, J=8.4 Hz, 1H), 7.25 (d, J=12.0 Hz, 1H), 7.16 (s, 1H), 4.95-4.92 (m, 1H), 4.77 (t, J=6.0 Hz, 2H), 4.54 (t, J=6.4 Hz, 2H), 4.04-3.99 (m, 2H), 3.94-3.89 (m, 2H), 2.23 (s, 3H). LCMS (M+H⁺) m/z: 540.2.

### Example 180: Preparation of 3-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)benzamide (Compound 180)

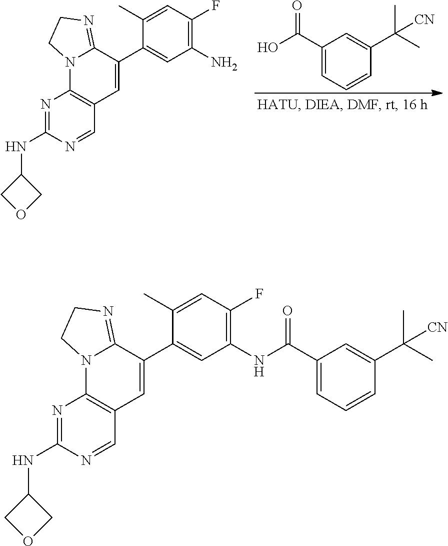 [SMILES: Cc1cc(F)c(N)cc1C1=Cc2cnc(NC3COC3)nc2N2CCN=C12 ; CC(C)(C#N)c1cccc(C(=O)O)c1 ; Cc1cc(F)c(NC(=O)c2cccc(C(C)(C)C#N)c2)cc1C1=Cc2cnc(NC3COC3)nc2N2CCN=C12]

### Step 1: Synthesis of 3-(2-Cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)benzamide

HATU (77.8 mg, 0.20 mmol) was added to the mixture of 6-(5-amino-4-fluoro-2-methylphenyl)-N-(oxetan-3-yl)-8,9-dihydroimidazo[1′,2′:1,6]pyrido[2,3-d]pyrimidin-2-amine (50 mg, 0.14 mmol), 3-(2-cyanopropan-2-yl)benzoic acid (31 mg, 0.16 mmol), DIEA (54.2 mg, 0.42 mmol) in DMF (2 mL). The mixture was stirred at rt for 16 hours. The reaction mixture was diluted with water (50 mL), filtered. The solid obtained was purified by Prep-HPLC (0.1% NH₃·H₂O) to afford 3-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)benzamide (12.8 mg, 17.5% yield) as a yellow solid. ¹H NMR (400 MHz, DMSO-d₆): δ 10.20 (s, 1H), 8.22 (d, J=4.8 Hz, 2H), 8.09 (s, 1H), 7.95 (d, J=8.0 Hz, 1H), 7.76 (d, J=7.8 Hz, 1H), 7.60 (t, J=8.0 Hz, 1H), 7.40 (d, J=8.0 Hz, 1H), 7.21 (d, J=11.2 Hz, 1H), 7.14 (s, 1H), 4.95-4.92 (m, 1H), 4.77 (t, J=6.4 Hz, 2H), 4.54 (t, J=6.4 Hz, 2H), 4.01 (t, J=8.8 Hz, 2H), 3.94-3.89 (m, 2H), 2.24 (s, 3H), 1.75 (s, 6H). LCMS (M+H⁺) m/z: 538.2.

### Example 181: Preparation of 4-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)picolinamide (Compound 181)

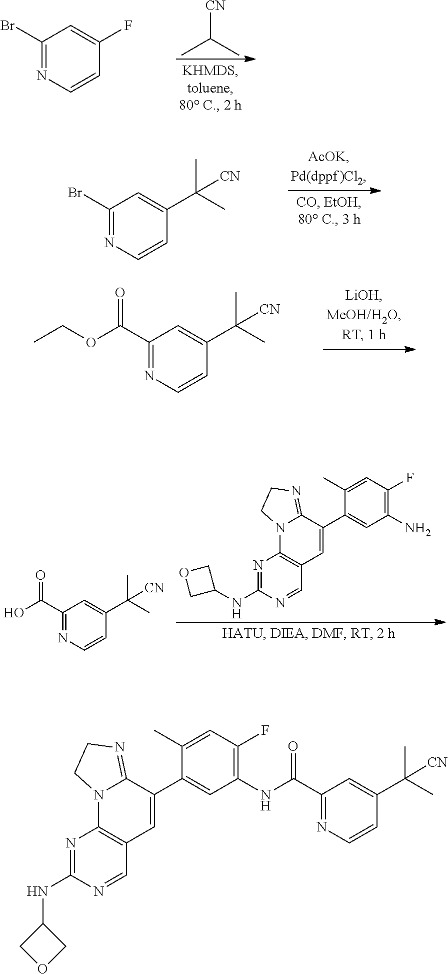

### Step 1: Synthesis of 2-(2-bromopyridin-4-yl)-2-methylpropanenitrile

To a solution of 2-bromo-4-fluoropyridine (2.0 g, 11.36 mmol) in toluene (20 mL) was added isobutyronitrile (784 mg, 1.00 mmol) and KHMDS (1.0 M, 11.4 mL, 11.36 mmol). The reaction mixture was stirred for 2 h at 80° C., quenched with NH₄Cl (aq.) (30 mL) and extracted with EA (30 mL×2). The combined organic phase was washed with brine (30 mL), dried with Na₂SO₄, filtered and concentrated in vacuum. The residue was purified by column chromatography (PE:EA=50:1) to afford 2-(2-bromopyridin-4-yl)-2-methylpropanenitrile (1.2 g, 47.1% yield) as a yellow solid. ¹H NMR (400 MHz, CDCl₃): δ 8.41 (d, J=5.2 Hz, 1H), 7.59 (d, J=1.2 Hz, 1H), 7.37-7.37 (m, 1H), 1.74 (s, 6H).

### Step 2: Synthesis of ethyl 4-(2-cyanopropan-2-yl)picolinate

To a solution of 2-(2-bromopyridin-4-yl)-2-methylpropanenitrile (1.2 g, 5.33 mmol) in EtOH (20 mL) was added AcOK (1.6 g, 16.00 mmol) and Pd(dppf)Cl₂ (390 mg, 0.53 mmol). The resulting mixture was stirred at 80° C. for 3 h under CO. The reaction mixture was diluted with water (10 mL) and extracted with EA (10 mL×3). The combined organic phase was washed with brine (10 mL), dried with Na₂SO₄, filtered and concentrated in vacuum. The residue was purified by column chromatography (PE:EA=3:1) to afford ethyl 4-(2-cyanopropan-2-yl)picolinate (900 mg, 74.3% yield) as a red solid. LCMS (M+H⁺) m/z: 219.1.

### Step 3: Synthesis of 4-(2-cyanopropan-2-yl)picolinic acid

To a solution of ethyl 4-(2-cyanopropan-2-yl)picolinate (900 mg, 4.13 mmol) in MeOH/H₂O (10 mL/2 mL) was added LiOH (297 mg, 12.39 mmol), the reaction mixture was stirred for 1 h at RT. The reaction mixture was evaporated to remove MeOH, and extracted with EA. The aqueous phase was adjusted to pH=2-3 with 1N HCl, and extracted with DCM (20 mL×3). The combined organic phase was washed with brine (10 mL), dried with Na₂SO₄, filtered and concentrated in vacuum to afford 4-(2-cyanopropan-2-yl)picolinic acid (190 mg, 88.8% yield) as a white solid. LCMS (M+H⁺) m/z: 191.2.

### Step 4: Synthesis of 4-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)picolinamide

To a solution of 6-(5-amino-4-fluoro-2-methylphenyl)-N-(oxetan-3-yl)-8,9-dihydroimidazo[1′,2′:1,6]pyrido[2,3-d]pyrimidin-2-amine (80 mg, 0.22 mmol), 4-(2-cyanopropan-2-yl)picolinic acid (42 mg, 0.22 mmol), HATU (125 mg, 0.33 mmol) and DIEA (85 mg, 0.66 mmol) in DMF (5 mL) at RT. The reaction mixture was stirred at RT for 2 h. The reaction mixture was diluted with water (20 mL), filtered. The filter cake was purified by column chromatography (DCM:MeOH=20:1) to afford 4-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)picolinamide (25 mg, 21.4% yield) as a yellow solid. ¹H NMR (400 MHz, DMSO-d₆): δ 10.46 (s, 1H), 8.87 (d, J=5.2 Hz, 1H), 8.35-8.31 (m, 3H), 7.97-7.92 (m, 2H), 7.33-7.30 (m, 2H), 5.01 (s, 1H), 4.85-4.82 (m, 2H), 4.62-4.59 (m, 2H), 4.12-4.08 (m, 2H), 4.01-3.96 (m, 2H), 2.28 (s, 3H), 1.82 (s, 6H). LCMS (M+H⁺) m/z: 539.2.

### Example 182: Preparation of 6-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)picolinamide (Compound 182)

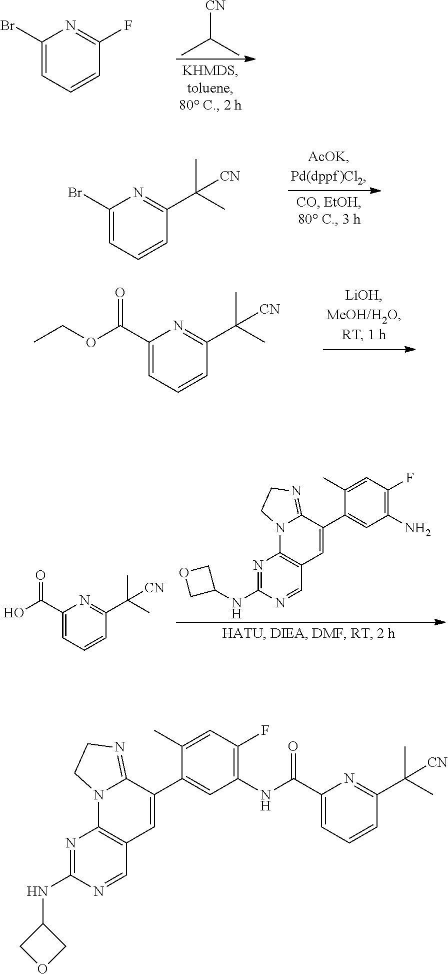

### Step 1: Synthesis of 2-(6-bromopyridin-2-yl)-2-methylpropanenitrile

To a solution of 2-bromo-6-fluoropyridine (176 mg, 1.00 mmol) in toluene (5 mL) was added isobutyronitrile (69 mg, 1.00 mmol) and KHMDS (1.0 M, 1.0 mL, 1.00 mmol). The reaction mixture was stirred for 2 h at 80° C., quenched with NH₄Cl (aq.) (10 mL) and extracted with EA (10 mL×2). The combined organic phase was washed with brine (10 mL), dried with Na₂SO₄, filtered and concentrated in vacuum. The residue was purified by Prep-TLC (PE:EA=2:1) to afford 2-(6-bromopyridin-2-yl)-2-methylpropanenitrile (100 mg, 44.4% yield) as a white solid. ¹H NMR (400 MHz, DMSO-d₆): δ 7.61-7.56 (m, 2H), 7.44-7.42 (m, 1H), 1.75 (s, 6H).

### Step 2: Synthesis of ethyl 6-(2-cyanopropan-2-yl)picolinate

To a solution of 2-(6-bromopyridin-2-yl)-2-methylpropanenitrile (160 mg, 0.71 mmol) in EtOH (10 mL) was added AcOK (209 mg, 2.13 mmol) and Pd(dppf)Cl₂ (52 mg, 0.071 mmol). The resulting mixture was stirred at 80° C. for 3 h under CO. The reaction mixture was diluted with water (10 mL) and extracted with EA (10 mL×3). The combined organic phase was washed with brine (10 mL), dried with Na₂SO₄, filtered and concentrated in vacuum. The residue was purified by column chromatography (PE:EA=10:1) to afford ethyl 6-(2-cyanopropan-2-yl)picolinate (135 mg, 87.3% yield) as a white solid. LCMS (M+H⁺) m/z: 219.2.

### Step 3: Synthesis of 6-(2-cyanopropan-2-yl)picolinic acid

To a solution of ethyl 6-(2-cyanopropan-2-yl)picolinate (220 mg, 1.01 mmol) in MeOH/H₂O (10 mL/2 mL) was added LiOH (72 mg, 3.03 mmol). The reaction mixture was stirred for 1 h at RT. The reaction mixture was evaporated to remove MeOH and extracted with EA. The aqueous phase was adjusted to pH=2-3 with 1N HCl, and extracted with DCM (10 mL×3). The combined organic phase was washed with brine (10 mL), dried with Na₂SO₄, filtered and concentrated in vacuum to afford 6-(2-cyanopropan-2-yl)picolinic acid (190 mg, 88.8% yield) as a white solid. LCMS (M+H⁺) m/z: 191.1.

### Step 4: Synthesis of 6-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)picolinamide

A mixture of 6-(5-amino-4-fluoro-2-methylphenyl)-N-(oxetan-3-yl)-8,9-dihydroimidazo[1′,2′:1,6]pyrido[2,3-d]pyrimidin-2-amine (70 mg, 0.19 mmol), 6-(2-cyanopropan-2-yl)picolinic acid (36 mg, 0.19 mmol), HATU (109 mg, 0.29 mmol) and DIEA (74 mg, 0.57 mmol) in DMF (5 mL) was stirred at r.t. for 2 h. The reaction mixture was diluted with water (20 mL), filtered. The filter cake was triturated with MeOH (5 mL), filtered. The solid was dried to afford 6-(2-cyanopropan-2-yl)-N-(2-fluoro-4-methyl-5-(2-(oxetan-3-ylamino)-8,9-dihydroimidazo[1′,2′:1,6]pyrido[2,3-d]pyrimidin-6-yl)phenyl)picolinamide (19.3 mg, 18.9% yield) as a white solid. ¹H NMR (400 MHz, DMSO-d₆): δ 10.33 (s, 1H), 8.24-8.16 (m, 3H), 8.13 (d, J=7.6 Hz, 1H), 7.98 (d, J=8.0 Hz, 1H), 7.93 (d, J=7.6 Hz, 1H), 7.26 (d, J=11.6 Hz, 1H), 7.17 (s, 1H), 4.95 (br, 1H), 4.77 (s, 2H), 4.55 (m, 2H), 4.02 (t, J=9.6 Hz, 2H), 3.94 (J=8.4 Hz, 2H), 2.23 (s, 3H), 1.82 (s, 6H). LCMS (M+H⁺) m/z: 539.2.

### Example 183: Preparation of N-(4-chloro-3-(2-(oxetan-3-ylamino)-8,9-dihydroimidazo[1′,2′:1,6]pyrido[2,3-d]pyrimidin-6-yl)phenyl)-3-(2-cyanopropan-2-yl)benzamide (Compound 183)

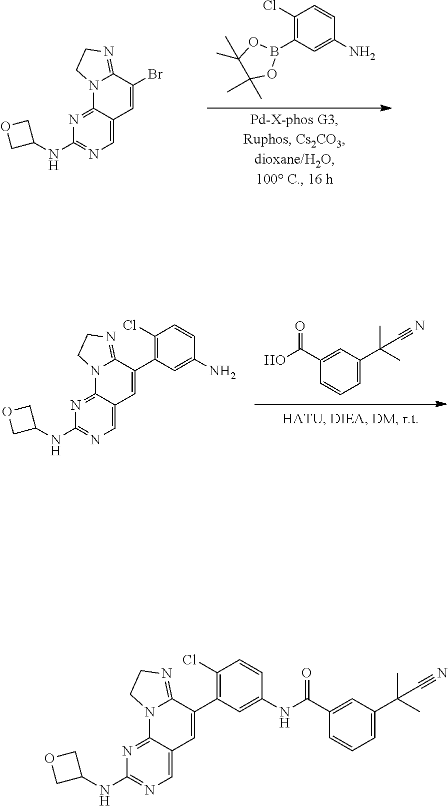 [SMILES: BrC1=Cc2cnc(NC3COC3)nc2N2CCN=C12 ; CC1(C)OB(c2cc(N)ccc2Cl)OC1(C)C ; Nc1ccc(Cl)c(C2=Cc3cnc(NC4COC4)nc3N3CCN=C23)c1 ; CC(C)(C#N)c1cccc(C(=O)O)c1 ; CC(C)(C#N)c1cccc(C(=O)Nc2ccc(Cl)c(C3=Cc4cnc(NC5COC5)nc4N4CCN=C34)c2)c1]

### Step 1: Synthesis of 6-(5-amino-2-chlorophenyl)-N-(oxetan-3-yl)-8, 9-dihydroimidazo[1′, 2′:1, 6]pyrido[2, 3-d]pyrimidin-2-amine

To a mixture of 6-bromo-N-(oxetan-3-yl)-8,9-dihydroimidazo[1′,2′:1,6]pyrido[2,3-d]pyrimidin-2-amine (500 mg, 1.56 mmol, 1.0 eq) and 4-chloro-3-(4,4,5,5-tetramethyl-1,3,2-dioxaborolan-2-yl)aniline (512 mg, 2.02 mmol, 1.3 eq) in dioxane/H₂O (25/5 mL), was added Pd(G)3 (132 mg, 0.156 mmol, 0.1 eq) and Cs₂CO₃ (1.5 g, 4.68 mmol, 3.0 eq), Ru-phos (73 mg, 0.156 mmol, 0.1 eq). The mixture was stirred at 100° C. under N₂ for 16 h. Concentration in vacuum and purification on silica gel column chromatography (DCM/MeOH=10:1) gave 6-(5-amino-2-chlorophenyl)-N-(oxetan-3-yl)-8, 9-dihydroimidazo[1′, 2′:1, 6]pyrido[2, 3-d]pyrimidin-2-amine (320 mg, 55.7% yield) as a yellow solid.

### Step 2: Synthesis of N-(4-chloro-3-(2-(oxetan-3-ylamino)-8,9-dihydroimidazo[1′,2′:1,6]pyrido[2,3-d]pyrimidin-6-yl)phenyl)-3-(2-cyanopropan-2-yl)benzamide

A mixture of 6-(5-amino-2-chlorophenyl)-N-(oxetan-3-yl)-8,9-dihydroimidazo[1′,2′:1,6]pyrido[2,3-d]pyrimidin-2-amine (60.0 mg, 0.16 mmol, 1.0 eq), 3-(2-cyanopropan-2-yl)benzoic acid (30.0 mg, 0.16 mmol, 1.0 eq) in DMF (5 mL) was added HATU (121.6 mg, 0.32 mmol, 2.0 eq) and DIEA (41.3 mg, 0.32 mmol, 3.0 eq). The mixture was stirred at RT under N₂ for 16 h. H₂O (20 mL) was added and the reaction mixture was extracted with EA twice. The combined extracts were washed with brine (20 mL). Concentration in vacuum and purification by Prep-HPLC (0.1% NH₄HCO₃) to afford N-(4-chloro-3-(2-(oxetan-3-ylamino)-8,9-dihydroimidazo[1′,2′:1,6]pyrido[2,3-d]pyrimidin-6-yl)phenyl)-3-(2-cyanopropan-2-yl)benzamide (5.0 mg, 12% yield) as a yellow solid. ¹H NMR (400 MHz, DMSO-d₆): δ 10.46 (s, 1H), 8.27 (d, J=5.6 Hz, 1H), 8.24 (s, 1H), 8.04 (s, 1H), 7.94 (d, J=8.0 Hz, 1H), 7.84-7.75 (m, 3H), 7.61 (t, J=7.6 Hz, 1H), 7.50 (d, J=8.4 Hz, 1H), 7.23 (s, 1H), 4.96 (br s, 1H), 4.77-4.76 (m, 2H), 4.56-4.53 (m, 2H), 4.01-3.99 (m, 2H), 3.93-3.91 (m, 2H, 1.73 (s, 6H). LCMS (M+H⁺) m/z: 540.1.

### Example 184: Preparation of N-(4-chloro-3-(2-(oxetan-3-ylamino)-8,9-dihydroimidazo[1′,2′:1,6]pyrido[2,3-d]pyrimidin-6-yl)phenyl)-4-(2-cyanopropan-2-yl)picolinamide

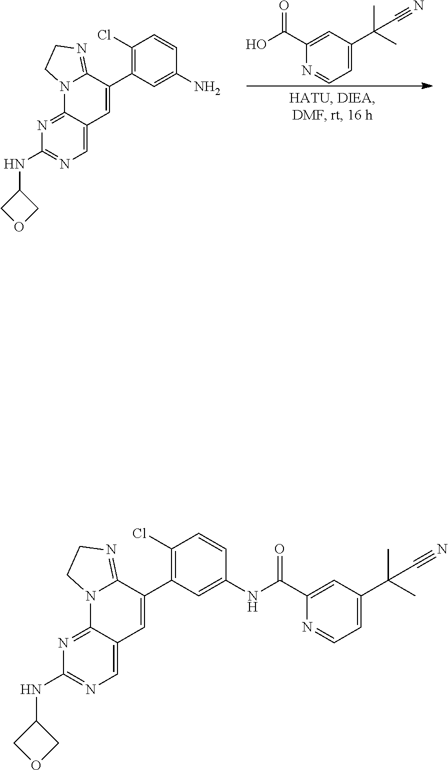 [SMILES: Nc1ccc(Cl)c(C2=Cc3cnc(NC4COC4)nc3N3CCN=C23)c1 ; CC(C)(C#N)c1ccnc(C(=O)O)c1 ; CC(C)(C#N)c1ccnc(C(=O)Nc2ccc(Cl)c(C3=Cc4cnc(NC5COC5)nc4N4CCN=C34)c2)c1]

### Step 1: Synthesis of N-(4-chloro-3-(2-(oxetan-3-ylamino)-8,9-dihydroimidazo[1′,2′:1,6]pyrido[2,3-d]pyrimidin-6-yl)phenyl)-4-(2-cyanopropan-2-yl)picolinamide

A mixture of 6-(5-amino-2-chlorophenyl)-N-(oxetan-3-yl)-8,9-dihydroimidazo[1′,2′:1,6]pyrido[2,3-d]pyrimidin-2-amine (100 mg, 0.29 mmol, 1.1 eq) and 4-(2-cyanopropan-2-yl)picolinic acid (50 mg, 0.27 mmol, and 1.0 eq) in DMF (3 mL) was added TEA (0.2 mL) and HATU (152 mg, 0.4 mmol, 1.5 eq). The mixture was stirred at 20° C. overnight. LCMS showed the reaction was OK. H₂O (20 mL) was added and the reaction mixture was extracted with EA twice. The combined extracts were washed with brine (20 mL). Concentration in vacuum and purification by Prep-HPLC (0.1% NH₄CO₃) to afford N-(4-chloro-3-(2-(oxetan-3-ylamino)-8,9-dihydroimidazo[1′,2′:1,6]pyrido[2,3-d]pyrimidin-6-yl)phenyl)-4-(2-cyanopropan-2-yl)picolinamide (38.9 mg, 27.1% yield) as a yellow solid. ¹H NMR (400 MHz, DMSO-d₆): δ 10.88 (s, 1H), 8.80 (d, J=5.2 Hz, 1H), 8.29-8.25 (m, 3H), 8.05 (s, 1H), 7.94-7.91 (m, 1H), 7.86-7.84 (m, 1H), 7.50 (d, J=8.8 Hz, 1H), 7.23 (s, 1H), 4.96-4.93 (m, 1H), 4.77-4.73 (m, 2H), 4.56-4.52 (m, 2H), 4.04-3.91 (m, 4H), 1.76 (s, 6H). LCMS (M+H⁺) m/z: 541.15

### Example 185: Preparation of N-(2-chloro-3-(2-(oxetan-3-ylamino)-8,9-dihydroimidazo[1′,2′:1,6]pyrido[2,3-d]pyrimidin-6-yl)phenyl)-4-(2-cyanopropan-2-yl)picolinamide (Compound 185)

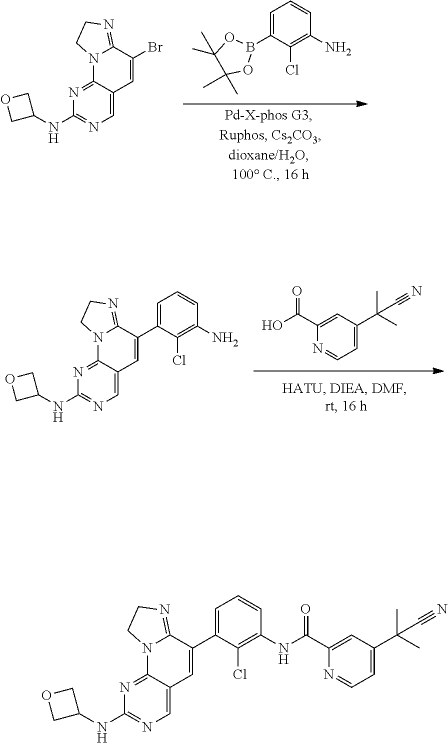 [SMILES: BrC1=Cc2cnc(NC3COC3)nc2N2CCN=C12 ; CC1(C)OB(c2cccc(N)c2Cl)OC1(C)C ; Nc1cccc(C2=Cc3cnc(NC4COC4)nc3N3CCN=C23)c1Cl ; CC(C)(C#N)c1ccnc(C(=O)O)c1 ; CC(C)(C#N)c1ccnc(C(=O)Nc2cccc(C3=Cc4cnc(NC5COC5)nc4N4CCN=C34)c2Cl)c1]

### Step 1: Synthesis of 6-(3-amino-2-chlorophenyl)-N-(oxetan-3-yl)-8, 9-dihydroimidazo[1′, 2′:1, 6]pyrido[2, 3-d]pyrimidin-2-amine

To a mixture of 6-bromo-N-(oxetan-3-yl)-8,9-dihydroimidazo[1′,2′:1,6]pyrido[2,3-d]pyrimidin-2-amine (500 mg, 1.56 mmol, 1.0 eq) and 2-chloro-3-(4,4,5,5-tetramethyl-1,3,2-dioxaborolan-2-yl)aniline (512 mg, 2.02 mmol, 1.3 eq) in dioxane/H₂O (25/5 mL), was added Pd(G)₃ (132 mg, 0.156 mmol, 0.1 eq) and Cs₂CO₃ (1.5 g, 4.68 mmol, 3.0 eq), Ru-phos (73 mg, 0.156 mmol, 0.1 eq). The mixture was stirred at 100° C. under N₂ for 16 h. Concentration in vacuum and purification on silica gel column chromatography (DCM/MeOH=10:1) afforded 6-(3-amino-2-chlorophenyl)-N-(oxetan-3-yl)-8, 9-dihydroimidazo[1′, 2′:1, 6]pyrido[2, 3-d]pyrimidin-2-amine (410 mg, 71.4% yield) as a yellow solid.

### Step 2: Synthesis of N-(2-chloro-3-(2-(oxetan-3-ylamino)-8, 9-dihydroimidazo[1′,2′:1,6]pyrido[2,3-d]pyrimidin-6-yl)phenyl)-4-(2-cyanopropan-2-yl)picolinamide

To a mixture of 6-(3-amino-2-chlorophenyl)-N-(oxetan-3-yl)-8, 9-dihydroimidazo[1′, 2′:1, 6]pyrido[2, 3-d]pyrimidin-2-amine (100 mg, 0.27 mmol, 1.0 eq) and 4-(2-cyanopropan-2-yl)picolinic acid (57 mg, 0.30 mmol, and 1.1 eq) in DMF (3 mL) was added DIEA (0.1 mL) and HATU (154 mg, 0.41 mmol, 1.5 eq). The mixture was stirred at 20° C. overnight. LCMS showed the reaction was OK. H₂O (20 mL) was added, the reaction mixture was extracted with EA (20 mL) twice. The combined extracts were washed with brine (20 mL). Concentration and purification by prep-HPLC (0.1% NH₄CO₃) gave N-(2-chloro-3-(2-(oxetan-3-ylamino)-8, 9-dihydroimidazo[1′,2′:1,6]pyrido[2,3-d]pyrimidin-6-yl)phenyl)-4-(2-cyanopropan-2-yl)picolinamide (14.0 mg, 9.6% yield) as a yellow solid. ¹H NMR (400 MHz, CD₃OD-d₄): δ 8.75 (d, J=5.2 Hz, 1H), 8.57 (d, J=8.0 Hz, 1H), 8.42 (s, 1H), 8.30 (s, 1H), 7.82-7.80 (m, 1H), 7.45 (t, J=8.0 Hz, 1H), 7.34 (s, 1H), 7.22 (d, J=7.6 Hz, 1H), 5.13 (t, J=7.2 Hz, 1H), 4.97-4.93 (m, 2H), 4.70-4.67 (m, 2H), 4.26-4.21 (m, 2H), 4.04-3.99 (m, 2H), 1.81 (s, 6H). LCMS (M+H⁺) m/z: 541.10.

### Example 186: Preparation of N-(4-chloro-2-fluoro-3-(2-(oxetan-3-ylamino)-8,9-dihydroimidazo[1′,2′:1,6]pyrido[2,3-d]pyrimidin-6-yl)phenyl)-3-(2-cyanopropan-2-yl)benzamide (Compound 186)

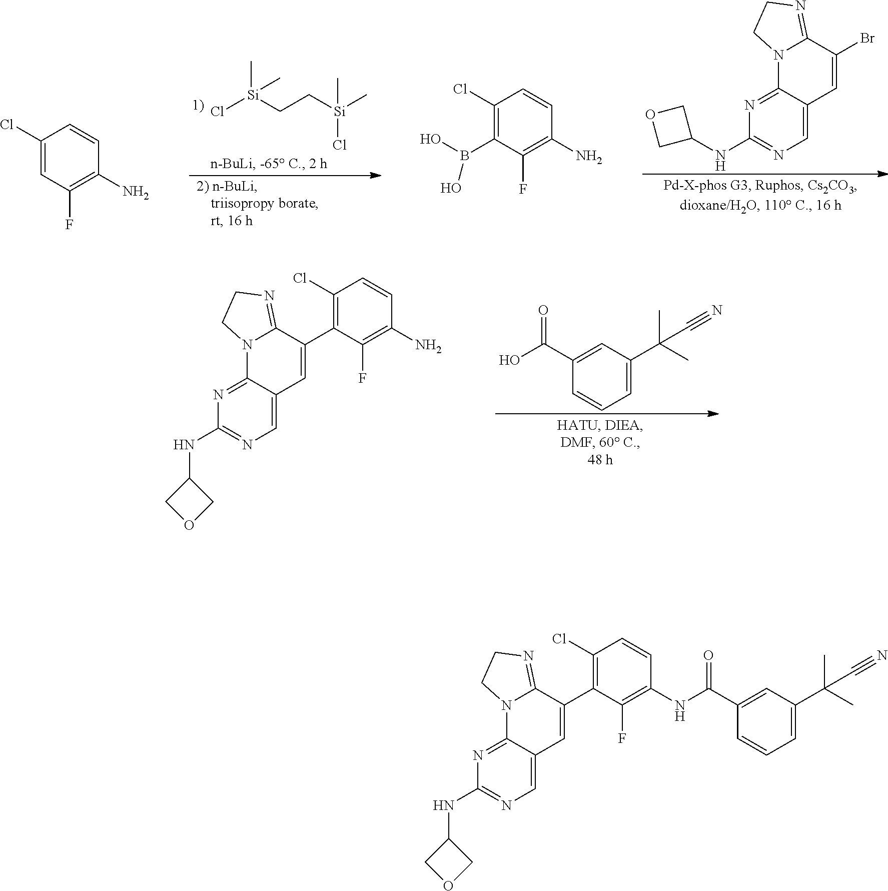

### Step 1: Synthesis of (3-amino-6-chloro-2-fluorophenyl)boronic acid

A solution of 4-chloro-2-fluoroaniline (2 g, 13.8 mmol) in THF (30 mL) was cooled to −65° C. and n-BuLi (15.18 mmol, 6 mL, 2.5 M in hexane) was added. After stirring at −65° C. for 30 mins. 1,2-bis(chlorodimethylsilyl)ethane (3115 mg, 14.09 mmol) in THF (8 mL) was added and after 20 mins at −65° C. a further portion of n-BuLi (15.18 mmol, 6 mL, 2.5 M in hexane) was added and the cooling bath was removed. After 20 mins at rt., the mixture was cooled to −65° C. and a further portion of n-BuLi (15.18 mmol, 6 mL, 2.5 M in hexane) was added and kept at −65° C. for 20 mins, then triisopropy borate (7783 mg, 41.4 mmol) was added and the cooling bath was removed followed by stirring at rt under N₂ for 16 h. The reaction was quenched by water and the pH was adjust to 2.0 by 1M HCl, extracted with EA. The organic phase was washed with sat. NaHCO₃, brine and concentrated. The residue was purified by flash chromatography to get (3-amino-6-chloro-2-fluorophenyl)boronic acid (570 mg, 22% yield) as a white solid. LCMS (M+H+) m/z: 189.9.

### Step 2: Synthesis of 6-(3-amino-6-chloro-2-fluorophenyl)-N-(oxetan-3-yl)-8,9-dihydroimidazo[1′,2′:1,6]pyrido[2,3-d]pyrimidin-2-amine

A mixture of (3-amino-6-chloro-2-fluorophenyl)boronic acid (200 mg, 1.06 mmol), 6-bromo-N-(oxetan-3-yl)-8,9-dihydroimidazo[1′,2′:1,6]pyrido[2,3-d]pyrimidin-2-amine (341 mg, 1.06 mmol), Cs2CO3 (1033 mg, 3.18 mmol) and Pd(G³) (89 mg, 0.106 mmol) and Ruphos (99 mg, 0.212 mmol) in dioxane (8 mL) and water (2 mL) was stirred at 100° C. under N2 for 2 h. The reaction mixture was cooled to r.t. and concentrated. The residue was purified by silica gel chromatography (DCM/MeOH=20/1) to get 6-(3-amino-6-chloro-2-fluorophenyl)-N-(oxetan-3-yl)-8,9-dihydroimidazo[1′,2′:1,6]pyrido[2,3-d]pyrimidin-2-amine (40 mg, 10% yield) as a yellow solid. LCMS (M+H⁺) m/z: 387.3.

### Step 3: Synthesis of N-(4-chloro-2-fluoro-3-(2-(oxetan-3-ylamino)-8,9-dihydroimidazo[1′,2′:1,6]pyrido[2,3-d]pyrimidin-6-yl)phenyl)-3-(2-cyanopropan-2-yl)benzamide

To a solution of 6-(3-amino-6-chloro-2-fluorophenyl)-N-(oxetan-3-yl)-8,9-dihydroimidazo[1′,2′:1,6]pyrido[2,3-d]pyrimidin-2-amine (40 mg, 0.124 mmol), 3-(2-cyanopropan-2-yl)benzoic acid (23 mg, 0.124 mmol) and DIEA (0.5 mL) in DMF (3 mL) was added HATU (141 mg, 0.372 mmol). The reaction solution was stirred at 60° C. under N₂ for 48 h. The solution was diluted with EA and washed with brine. The organic phase was concentrated and purified by prep-TLC (DCM/MeOH=10/1 with 0.1% TEA) to get crude product, which was purified by prep-HPLC (0.10%/NH₄HCO₃/CH₃CN/H₂O) to afford N-(4-chloro-2-fluoro-3-(2-(oxetan-3-ylamino)-8,9-dihydroimidazo[1′,2′:1,6]pyrido[2,3-d]pyrimidin-6-yl)phenyl)-3-(2-cyanopropan-2-yl)benzamide (1.4 mg, 2% yield) as a yellow solid. ¹H NMR (400 MHz, DMSO-d₆): δ 10.33 (s, 1H), 8.34 (d, J=5.2 Hz, 1H), 8.26 (s, 1H), 8.09 (s, 1H), 7.94 (d, J=8.8 Hz, 1H), 7.77 (d, J=6.4 Hz, 1H), 7.70 (t, J=8.4 Hz, 1H), 7.60 (t, J=8.0 Hz, 1H), 7.44 (d, J=8.8 Hz, 1H), 7.29 (s, 1H), 5.04-4.87 (m, 1H), 4.76-4.74 (m, 2H), 4.56-4.53 (m, 2H), 4.05-3.99 (m, 2H), 3.91-3.87 (m, 2H), 1.74 (s, 6H). LCMS (M+H⁺) m/z: 558.0.

### Example 187: Preparation of 3-fluoro-N-(4-methyl-3-(2-(oxetan-3-ylamino)-8,9-dihydroimidazo[1′,2′:1,6]pyrido[2,3-d]pyrimidin-6-yl)phenyl)-4-(trifluoromethyl)picolinamide (Compound 187)

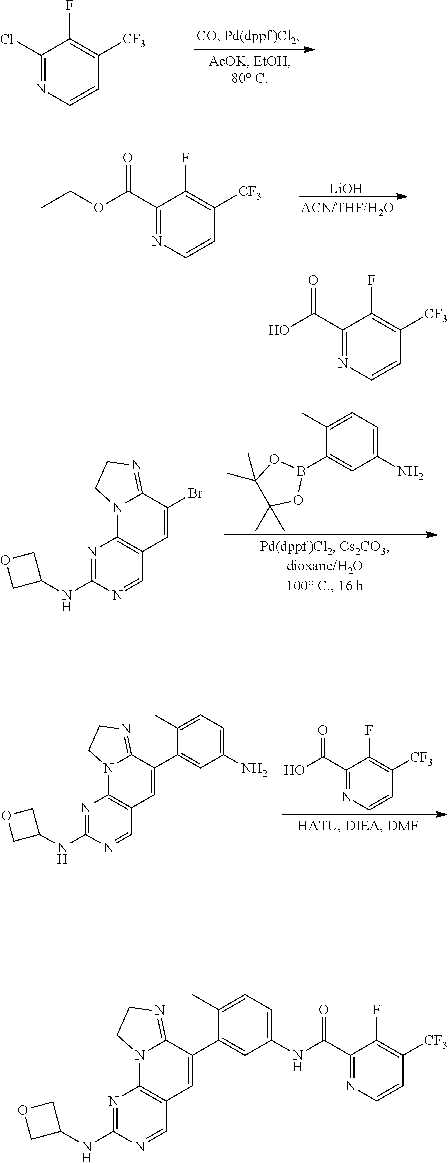

### Step 1: Synthesis of ethyl 3-fluoro-4-(trifluoromethyl)picolinate

A mixture of 2-chloro-3-fluoro-4-(trifluoromethyl)pyridine (500 mg, 2.51 mmol), AcOK (492 mg, 5.02 mmol) and [1,1′-Bis(diphenylphosphino)ferrocene]dichloropalladium(II) (183 mg, 0.25 mmol) in EtOH (30.0 mL) was degassed, charged with CO three times and stirred at 80° C. for 16 h. The reaction mixture was filtered and concentrated. The residue was purified by column chromatography on silica gel (PE:EA=10:1) to afford ethyl 3-fluoro-4-(trifluoromethyl)picolinate (513 mg, 86% yield) as a white solid. LCMS (M+H⁺) m/z: 238.0.

### Step 2: Synthesis of 3-fluoro-4-(trifluoromethyl)picolinic acid

A solution of ethyl 3-fluoro-4-(trifluoromethyl)picolinate (513 mg, 2.16 mmol) and LiOH·H₂O (454 mg, 10.8 mmol) in THF (10.0 mL), CH3CN (10.0 mL) and H₂O (10.0 mL) was stirred at 25° C. for 2.5 h. Then 2N HCl was added, the pH was adjusted to 5˜6. The reaction mixture was extracted with EA (50 mL×2). The combined organic phase was washed with brine, dried over Na2SO4, filtered and concentrated to afford 3-fluoro-4-(trifluoromethyl)picolinic acid (447 mg, 99% yield) as a white solid. LCMS (M+H⁺) m/z: 210.0.

### Step 3: Synthesis of 6-(5-amino-2-methylphenyl)-N-(oxetan-3-yl)-8,9-dihydroimidazo[1′,2′:1,6]pyrido[2,3-d]pyrimidin-2-amine

A mixture of 6-bromo-N-(oxetan-3-yl)-8,9-dihydroimidazo[1′,2′:1,6]pyrido[2,3-d]pyrimidin-2-amine (460 mg, 1.43 mmol), 4-methyl-3-(4,4,5,5-tetramethyl-1,3,2-dioxaborolan-2-yl)aniline (333 mg, 1.43 mmol), Cs₂CO₃ (932 mg, 2.86 mmol) and [1,1′-Bis(diphenylphosphino)ferrocene]dichloropalladium(II) (52 mg, 0.07 mmol) in dioxane (8.0 mL) and H₂O (0.8 mL) was stirred at 100° C. for 16 h under N₂. The reaction mixture was concentrated and purified by column chromatography on silica gel (DCM:MeOH=20:1, +0.1% NH₃-MeOH), then triturated with EA (4.0 mL) to afford 6-(5-amino-2-methylphenyl)-N-(oxetan-3-yl)-8,9-dihydroimidazo[1′,2′:1,6]pyrido[2,3-d]pyrimidin-2-amine (364 mg, 73% yield) as a yellow solid. LCMS (M+H⁺) m/z: 349.0.

### Step 4: Synthesis of 3-fluoro-N-(4-methyl-3-(2-(oxetan-3-ylamino)-8,9-dihydroimidazo[1′,2′:1,6]pyrido[2,3-d]pyrimidin-6-yl)phenyl)-4-(trifluoromethyl)picolinamide

To a solution of 3-fluoro-4-(trifluoromethyl)picolinic acid (44 mg, 0.211 mmol), 6-(5-amino-2-methylphenyl)-N-(oxetan-3-yl)-8,9-dihydroimidazo[1′,2′:1,6]pyrido[2,3-d]pyrimidin-2-amine (70 mg, 0.201 mmol) and HATU (115 mg, 0.301 mmol) in DMF (4.0 mL), was added DIEA (52 mg, 0.402 mmol). The resulting mixture was stirred at r.t. for 1.5 h under N₂. Water was added, the reaction mixture was extracted with DCM (40 mL×3). The combined organic phase was washed with brine, dried over Na₂SO₄, filtered and concentrated. The residue was purified by column chromatography on silica gel (DCM/MeOH=20:1, +0.1% TEA) to afford 3-fluoro-N-(4-methyl-3-(2-(oxetan-3-ylamino)-8,9-dihydroimidazo[1′,2′:1,6]pyrido[2,3-d]pyrimidin-6-yl)phenyl)-4-(trifluoromethyl)picolinamide (59.1 mg, 55% yield) as a yellow solid. ¹H NMR (400 MHz, DMSO-d₆): δ 10.73 (s, 1H), 8.80 (d, J=4.8 Hz, 1H), 8.27 (s, 2H), 8.09 (t, J=4.8 Hz, 1H), 7.70 (d, J=2.0 Hz, 1H), 7.64 (dd, J=8.0, 2.0 Hz, 1H), 7.25-7.23 (m, 2H), 4.97 (br, 1H), 4.77 (t, J=5.6 Hz, 2H), 4.55 (t, J=6.0 Hz, 2H), 4.05-4.03 (m, 2H), 3.95-3.90 (m, 2H), 2.19 (s, 3H). LCMS (M+H⁺) m/z: 540.4.

### Example 188 and 189: Preparation of N-(4-bromo-2-fluoro-5-(2-(oxetan-3-ylamino)-8,9-dihydroimidazo[1′,2′:1,6]pyrido[2,3-d]pyrimidin-6-yl)phenyl)-4-(trifluoromethyl)picolinamide (Compound 188) and N-(6-(2-bromo-4-fluoro-5-(4-(trifluoromethyl)picolinamido)phenyl)-8,9-dihydroimidazo[1′,2′:1,6]pyrido[2,3-d]pyrimidin-2-yl)-N-(oxetan-3-yl)-4-(trifluoromethyl)picolinamide (Compound 189)

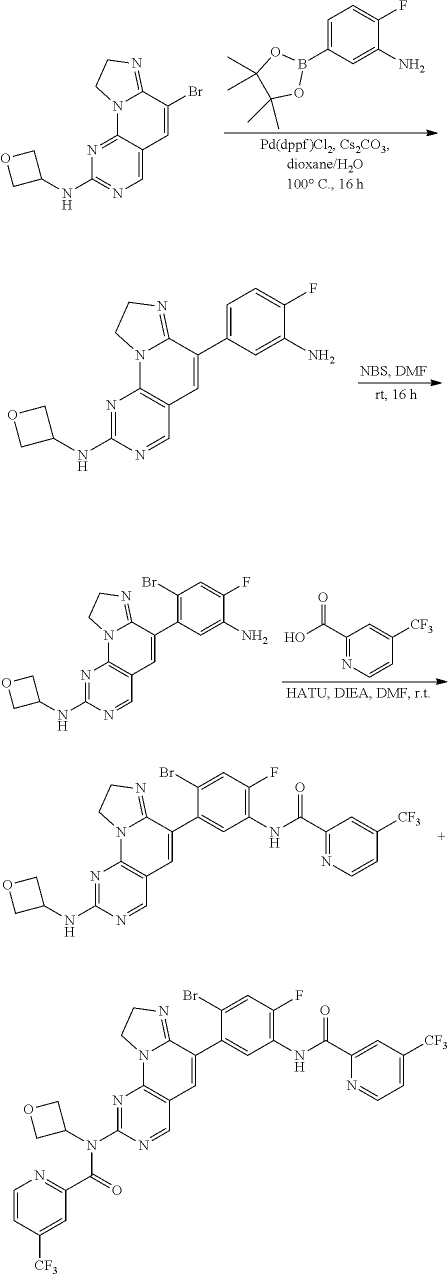

### Step 1: Synthesis of 6-(3-amino-4-fluorophenyl)-N-(oxetan-3-yl)-8,9-dihydro imidazo[1′,2′:1,6]pyrido[2,3-d]pyrimidin-2-amine

A mixture of 6-bromo-N-(oxetan-3-yl)-8,9-dihydroimidazo[1′,2′:1,6]pyrido[2,3-d]pyrimidin-2-amine (700 mg, 2.5 mmol, 1.0 eq) and 2-fluoro-5-(4,4,5,5-tetramethyl-1,3,2-dioxaborolan-2-yl)aniline (711 mg, 3 mmol, 1.2 eq) in dioxane/H₂O (25/5 mL), Pd(dppf)Cl₂ (102 mg, 0.13 mmol, 0.05 eq) and Cs₂CO₃ (2.4 g, 7.5 mmol, 3.0 eq) was stirred at 110° C. under N₂ for 16 h. Concentration and purification by column chromatography (DCM/MeOH=10:1) gave 6-(3-amino-4-fluorophenyl)-N-(oxetan-3-yl)-8,9-dihydroimidazo[1′,2′:1,6]pyrido[2,3-d]pyrimidin-2-amine (750 mg, 80% yield) as a yellow solid.

### Step 2: Synthesis of 6-(5-amino-2-bromo-4-fluorophenyl)-N-(oxetan-3-yl)-8,9-di hydroimidazo[1′,2′:1,6]pyrido[2,3-d]pyrimidin-2-amine

To a solution of 6-(3-amino-4-fluorophenyl)-N-(oxetan-3-yl)-8,9-dihydroimidazo[1′,2′:1,6]pyrido[2,3-d]pyrimidin-2-amine (750 mg, 2.1 mmol, 1.0 eq) in DMF (5 mL) was added NBS (256 mg, 1.5 mmol, 0.7 eq). The mixture was stirred under N₂ at 0° C. for 2 h. LCMS show the reaction was OK. Concentration and purification by column chromatography gave 6-(5-amino-2-bromo-4-fluorophenyl)-N-(oxetan-3-yl)-8,9-di hydroimidazo[1′,2′:1,6]pyrido[2,3-d]pyrimidin-2-amine (410 mg, 50% yield) as a yellow solid.

### Step 3: Synthesis of N-(4-bromo-2-fluoro-5-(2-(oxetan-3-ylamino)-8,9-dihydro imidazo[1′,2′:1,6]pyrido[2,3-d]pyrimidin-6-yl)phenyl)-4-(trifluoromethyl)picolinamide and N-(6-(2-bromo-4-fluoro-5-(4-(trifluoromethyl)picolinamido)phenyl)-8,9-dihydroimidazo[1′,2′:1,6]pyrido[2,3-d]pyrimidin-2-yl)-N-(oxetan-3-yl)-4-(trifluoromethyl)picolinamide

A mixture of 6-(5-amino-2-bromo-4-fluorophenyl)-N-(oxetan-3-yl)-8,9-di hydroimidazo[1′,2′:1,6]pyrido[2,3-d]pyrimidin-2-amine (100 mg, 0.3 mmol, 1.0 eq) and 4-(trifluoromethyl)picolinic acid (115 mg, 0.6 mmol, 2.0 eq) in DMF (3 mL) was added TEA (0.2 mL) and HATU (286 mg, 0.9 mmol, 2.0 eq). The mixture was stirred at 20° C. overnight. LCMS show the reaction was OK. Water was added, the reaction mixture was extracted with EA. The combined extracts were washed with brine. Concentration and purification by prep-HPLC (0.1% NH₃·H₂O) gave N-(4-bromo-2-fluoro-5-(2-(oxetan-3-ylamino)-8,9-dihydro imidazo[1′,2′:1,6]pyrido[2,3-d]pyrimidin-6-yl)phenyl)-4-(trifluoromethyl)picolinamide (4.5 mg, 3.8%) and N-(6-(2-bromo-4-fluoro-5-(4-(trifluoromethyl)picolinamido)phenyl)-8,9-dihydroimidazo[1′,2′:1,6]pyrido[2,3-d]pyrimidin-2-yl)-N-(oxetan-3-yl)-4-(trifluoromethyl)picolinamide (5.9 mg, 5.0%) as a yellow solid. Compound 188: ¹H NMR (400 MHz, DMSO-d₆): δ 10.60 (s, 1H), 9.06 (d, J=4.8 Hz, 1H), 8.37-8.25 (m, 3H), 8.15-8.14 (m, 1H), 8.04-8.02 (m, 1H), 7.84-7.81 (m, 1H), 7.75 (s, 1H), 4.97-4.94 (m, 1H), 4.77-4.75 (m, 2H), 4.56-4.54 (m, 2H), 4.09-3.88 (m, 4H). LCMS (M+H⁺) m/z: 604.0. Compound 189: ¹H NMR (400 MHz, DMSO-d₆): δ 10.60 (s, 1H), 9.06 (d, J=4.8 Hz, 1H), 8.68 (d, J=5.2 Hz, 1H), 8.32 (s, 1H), 8.26 (s, 1H), 8.15-8.10 (m, 2H), 8.01 (d, J=8 Hz, 1H), 7.87-7.82 (m, 2H), 7.32 (s, 1H), 5.39-5.35 (m, 1H), 4.83-4.81 (m, 4H), 3.89-3.84 (m, 2H), 3.62-3.57 (m, 2H). LCMS (M+H⁺) m/z: 777.0.

### Example 190: Preparation of N-(5-methyl-4-(2-(methylamino)-8,9-dihydroimidazo[1′,2′:1,6]pyrido[2,3-d]pyrimidin-6-yl)pyridin-2-yl)-3-(trifluoromethyl)benzamide (Compound 190)

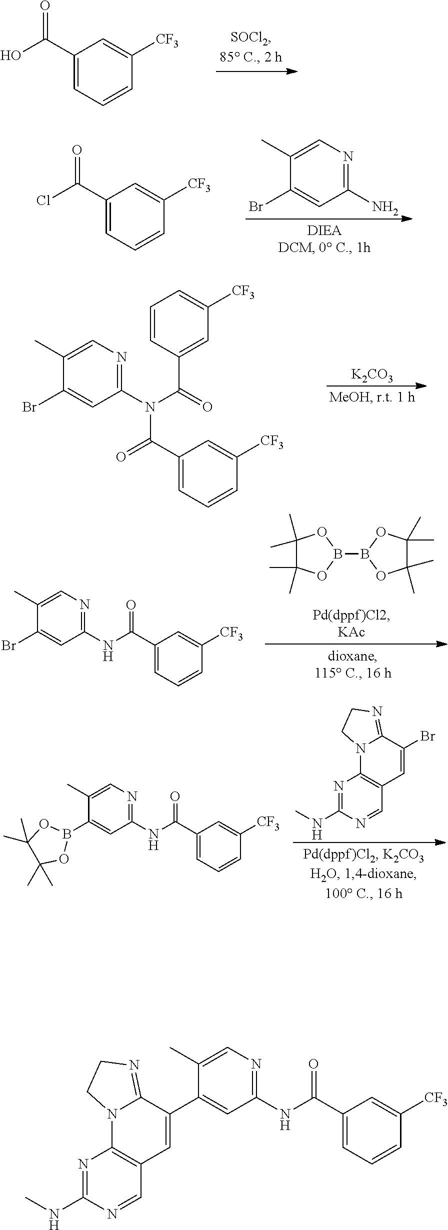

The preparation of 6-bromo-N-methyl-8,9-dihydroimidazo[1′,2′:1,6]pyrido[2,3-d]pyrimidin-2-amine was described in Example 133.

### Step 1: Synthesis of 3-(trifluoromethyl)benzoyl chloride

The mixture of 3-(trifluoromethyl)benzoic acid (300 mg, 1.58 mmol, 1.0 eq) in SOCl₂ (10 mL) was stirred at 80° C. for 2 h. The mixture was concentrated to afford product 3-(trifluoromethyl)benzoyl chloride (400 mg, crude) as yellow oil, which was used for the next step directly.

### Step 2: Synthesis of N-(4-bromo-5-methylpyridin-2-yl)-3-(trifluoromethyl)-N-(3-(trifluoromethyl)benzoyl)benzamide

To a cooled (0° C.) mixture of 4-bromo-5-methylpyridin-2-amine (300 mg, 1.6 mmol, 1.0 eq) and DIEA (2 mL) in DCM (20 mL) was added a solution of 3-(trifluoromethyl)benzoyl chloride (400 mg, crude) in DCM (3 mL). The mixture was stirred at r.t. for 1 h. Water was added, the reaction mixture was extracted with DCM. The combined extracts were washed with brine, concentrated to afford N-(4-bromo-5-methylpyridin-2-yl)-3-(trifluoromethyl)-N-(3-(trifluoromethyl)benzoyl)benzamide (500 mg, crude) as a yellow solid. which was used next step. LCMS (M+H⁺) m/z: 530.8.

### Step 3: Synthesis of N-(4-bromo-5-methylpyridin-2-yl)-3-(trifluoromethyl)benzamide

The mixture of N-(4-bromo-5-methylpyridin-2-yl)-3-(trifluoromethyl)-N-(3-(trifluoromethyl)benzoyl)benzamide (500 mg, crude) and K₂CO₃ (500 mg) in MeOH (20 mL) was stirred at r.t. for 1 h. The mixture was concentrated and diluted with by EA (50 mL) and H₂O (30 mL), aqueous phase was extracted by EA (50 mL) twice. The organic phase was washed with brine (50 mL), dried Na2SO4 and concentrated in vacuum to give crude product, which was purified on silica gel column flash chromatography (EtOAc:PE=1:3, v/v) to afford product N-(4-bromo-5-methylpyridin-2-yl)-3-(trifluoromethyl)benzamide (200 mg) as a yellow solid. LCMS (M−200)⁺ m/z: 358.9.

### Step 4: Synthesis of N-(5-methyl-4-(4,4,5,5-tetramethyl-1,3,2-dioxaborolan-2-yl)pyridin-2-yl)-3-(trifluoromethyl)benzamide

The mixture of N-(4-bromo-5-methylpyridin-2-yl)-3-(trifluoromethyl)benzamide (90 mg, 0.25 mmol, 1.0 eq), 4,4,4′,4′,5,5,5′,5′-octamethyl-2,2′-bi(1,3,2-dioxaborolane) (95 mg, 0.38 mmol, 1.5 eq), KOAc (49 mg, 0.5 mmol, 2 eq) and Pd(dppf)Cl₂ (30 mg) in dioxane (5 mL) was stirred at 115° C. for 16 h. The reaction was monitored by LCMS, and the reaction solution was used for the next step directly. LCMS ([M−200]⁺ m/z: 325.1.

### Step 5: Synthesis of N-(5-methyl-4-(2-(methylamino)-8,9-dihydroimidazo[1′,2′:1,6]pyrido[2,3-d]pyrimidin-6-yl)pyridin-2-yl)-3-(trifluoromethyl)benzamide

The mixture of N-(5-methyl-4-(4,4,5,5-tetramethyl-1,3,2-dioxaborolan-2-yl)pyridin-2-yl)-3-(trifluoromethyl)benzamide (reaction solution), 6-bromo-N-methyl-8,9-dihydroimidazo[1′,2′:1,6]pyrido[2,3-d]pyrimidin-2-amine (80 mg, 0.28 mmol, 1.0 eq), K2CO3 (118 mg, 0.86 mmol, 3.0 eq) and Pd(dppf)Cl2 (30 mg) was stirred at 110° C. for 16 h. Water was added, the reaction mixture was extracted with DCM. The organic phase was washed with brine (30 mL), dried Na₂SO₄ and concentrated in vacuum to give crude product, which was purified on silica gel column flash chromatography (DCM:MeOH=20:1, v/v) and Prep-HPLC (0.1% NH₄HCO₃) to afford N-(5-methyl-4-(2-(methylamino)-8,9-dihydroimidazo[1′,2′:1,6]pyrido[2,3-d]pyrimidin-6-yl)pyridin-2-yl)-3-(trifluoromethyl)benzamide (15 mg, 11% yield) as a yellow solid. ¹H NMR (400 MHz, DMSO-d6): δ 11.16 (s, 1H), 8.39 (s, 1H), 8.31-8.24 (m, 3H), 8.11 (s, 1H), 7.98-7.96 (m, 1H), 7.79-7.75 (m, 1H), 7.61-7.54 (m, 1H), 7.29 (s, 1H), 4.17-3.86 (m, 4H), 2.86 (s, 3H), 2.18 (s, 3H). LCMS (M+H⁺) m/z: 480.0.

### Example 191: Preparation of N-(6-methyl-5-(2-(methylamino)-8,9-dihydroimidazo[1′,2′:1,6]pyrido[2,3-d]pyrimidin-6-yl)pyridin-3-yl)-4-(trifluoromethyl)picolinamide (Compound 191)

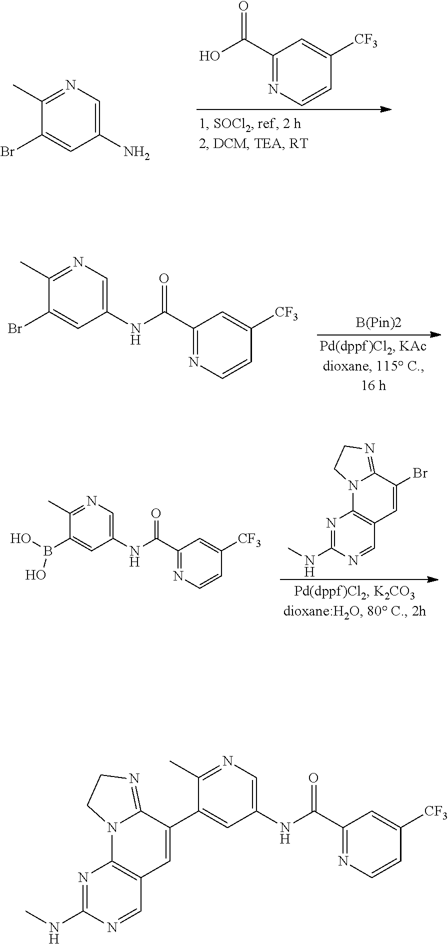

### Step 1: Synthesis of N-(5-bromo-6-methylpyridin-3-yl)-4-(trifluoromethyl)picolinamide

The mixture of 4-(trifluoromethyl)picolinic acid (191 mg, 1.0 mmol) in SOCl₂ (3 mL) was stirred at 80° C. for 1 h. The reaction was concentrated to give solid. The solid product was dissolved in DCM (2 mL), then it was added to the mixture of TEA (300 mg, 3.0 mmol), 5-bromo-6-methylpyridin-3-amine (187 mg, 1.0 mmol) in DCM (5 mL) at rt. The reaction was stirred at 25° C. for 3 hours. The reaction mixture was poured into water, extracted with DCM (30 mL×2). The combined organic phase was washed with brine, dried over with Na₂SO₄, concentrated and purified by flash to give N-(5-bromo-6-methylpyridin-3-yl)-4-(trifluoromethyl)picolinamide (170 mg, 47% yield) as white solid. LCMS (M+H⁺) m/z: 360.0.

### Step 2: Synthesis of (2-methyl-5-(4-(trifluoromethyl)picolinamido)pyridin-3-yl)boronic acid

A mixture N-(5-bromo-6-methylpyridin-3-yl)-4-(trifluoromethyl)picolinamide (72 mg, 0.2 mmol, 1.0 equiv), Pin₂B₂ (76 mg, 0.3 mmol, 1.5 equiv), KOAc (60 mg, 0.6 mmol, 3.0 equiv), and PdCl₂(dppf) (29 mg, 0.04 mmol, 20 mol %) in 1,4-dioxane (2 mL) was refluxed under N₂ for 3 h. The reaction solution was used to next step without further purification. LCMS (M+H⁺) m/z: 325.0.

### Step 3: Synthesis of N-(6-methyl-5-(2-(methylamino)-8,9-dihydroimidazo[1′,2′:1,6]pyrido[2,3-d]pyrimidin-6-yl)pyridin-3-yl)-4-(trifluoromethyl)picolinamide

(2-Methyl-5-(4-(trifluoromethyl)picolinamido)pyridin-3-yl)boronic acid (above solution, 0.2 mmol, 1.0 equiv), 6-bromo-N-methyl-8,9-dihydroimidazo[1′,2′:1,6]pyrido[2,3-d]pyrimidin-2-amine (54 mg, 0.2 mmol, 1.0 equiv), K₂CO₃ (82 mg, 0.6 mmol, 3.0 equiv), and PdCl₂ (dppf) (28 mg, 0.04 mmol, 20 mol %) in 1, 4-dioxane (3 mL) and H₂O (0.5 mL) was stirred at 90° C. for 2 h under N2. The reaction mixture was concentrated and purified by Prep-HPLC to give N-(6-methyl-5-(2-(methylamino)-8,9-dihydroimidazo[1′,2′:1,6]pyrido[2,3-d]pyrimidin-6-yl)pyridin-3-yl)-4-(trifluoromethyl)picolinamide (16.7 mg, 17% yield) as white solid. ¹H NMR (400 MHz, DMSO-d₆): δ 9.05 (d, J=5.2 Hz, 1H), 8.91 (d, J=2.8 Hz, 1H), 8.35 (s, 1H), 8.26-8.20 (m, 2H), 8.12 (d, J=4.8 Hz, 1H), 7.48 (s, 1H), 7.23 (s, 1H), 6.09 (s, 1H), 4.08-3.90 (m, 4H), 2.67 (s, 3H), 2.40 (s, 3H). LCMS (M+H⁺) m/z: 481.0.

### Example 192: Preparation of N-(5-methyl-6-(2-(methylamino)-8,9-dihydroimidazo[1′,2′:1,6]pyrido[2,3-d]pyrimidin-6-yl)pyridin-2-yl)-4-(trifluoromethyl)picolinamide (Compound 192)

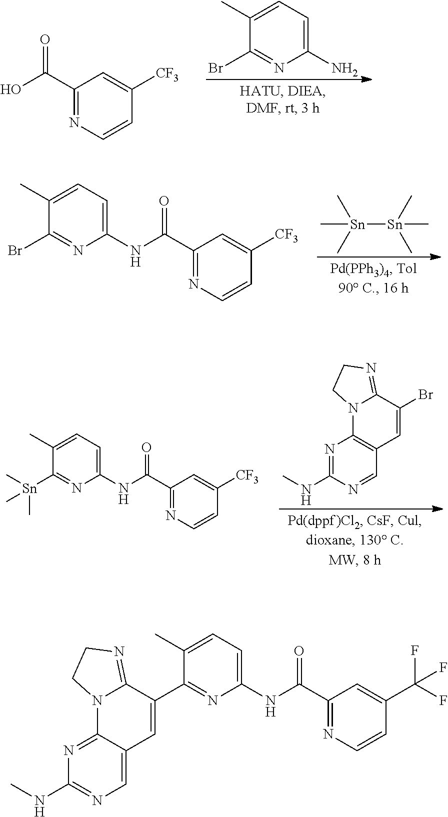

### Step 1: Synthesis of N-(6-bromo-5-methylpyridin-2-yl)-4-(trifluoromethyl)picolinamide

To a solution of 4-(trifluoromethyl)picolinic acid (700 mg, 1.90 mmol) in DMF (10 mL) was added 6-bromo-5-methylpyridin-2-amine (753 mg, 4.03 mmol), HATU (2.0 g, 5.49 mmol) and TEA (1.4 g, 10.9 mmol). The mixture was stirred at rt under N₂ for 16 h. The mixture was diluted with water (50 mL) and then extracted with EA (30 mL×3). The residue was purified on silica gel column chromatography (PE:EA=10:1) to afford N-(6-bromo-5-methylpyridin-2-yl)-4-(trifluoromethyl)picolinamide (750 mg, 57% yield) as a white solid. LCMS (M+H+) m/z: 361.9.

### Step 2: Synthesis of N-(5-methyl-6-(trimethylstannyl)pyridin-2-yl)-4-(trifluoromethyl)picolinamide

To a solution of N-(6-bromo-5-methylpyridin-2-yl)-4-(trifluoromethyl)picolinamide (150 mg, 0.42 mmol) in toluene (5 mL) was added 1,1,1,2,2,2-hexamethyldistannane (273 mg, 0.83 mmol), Pd(PPh₃)₄ (48 mg, 0.042 mmol). The mixture stirred at 90° C. under N₂ for 16 h. The mixture was concentrated, diluted with DCM (5 mL), filtered and the filtrate was concentrated to afford N-(5-methyl-6-(trimethylstannyl)pyridin-2-yl)-4-(trifluoromethyl)picolinamide (430 mg, crude) as a grey solid. LCMS (M+H⁺) m/z: 446.0.

### Step 3: Synthesis of N-(5-methyl-6-(2-(methylamino)-8,9-dihydroimidazo[1′,2′:1,6]pyrido[2,3-d]pyrimidin-6-yl)pyridin-2-yl)-4-(trifluoromethyl)picolinamide

To a solution of N-(5-methyl-6-(trimethylstannyl)pyridin-2-yl)-4-(trifluoromethyl)picolinamide (330 mg, 0.32 mmol) in 1,4-dioxane (3 mL) was added 6-bromo-N-methyl-8,9-dihydroimidazo[1′,2′:1,6]pyrido[2,3-d]pyrimidin-2-amine (107 mg, 0.38 mmol), Pd(dppf)Cl₂ (23 mg, 0.032 mmol), CuI (5 mg, 0.032 mmol) and CsF (6 mg, 0.75 mmol). The mixture was stirred at 130° C. under MW for 8 hours. The mixture was filtered and purified by prep-HPLC (0.1%/HCl/CH₃CN/H₂O) to afford N-(5-methyl-6-(2-(methylamino)-8,9-dihydroimidazo[1′,2′:1,6]pyrido[2,3-d]pyrimidin-6-yl)pyridin-2-yl)-4-(trifluoromethyl)picolinamide (50.4 mg, 33% yield) as a grey solid. ¹H NMR (400 MHz, DMSO-d₆): δ 10.95 (s, 1H), 10.19 (s, 1H), 9.09 (d, J=5.2 Hz, 1H), 8.94 (s, 1H), 8.63 (t, J=4.4 Hz, 1H), 8.50 (s, 1H), 8.44 (s, 1H), 8.18 (d, J=4.4 Hz, 1H), 8.12 (d, J=4.8 Hz, 1H), 7.99 (d, J=8.4 Hz, 1H), 4.68-4.55 (m, 2H), 4.17 (t, J=10.0 Hz, 2H), 2.98 (d, J=4.8 Hz, 3H), 2.45 (s, 3H). LCMS (M+H⁺) m/z: 481.4.

### Example 193: Preparation of 3-(4-fluorophenyl)-1-isopropyl-N-(4-methyl-3-(2-(methylamino)-8,9-dihydroimidazo[1′,2′:1,6]pyrido[2,3-d]pyrimidin-6-yl)phenyl)-2,4-dioxo-1,2,3,4-tetrahydropyrimidine-5-carboxamide (Compound 193)

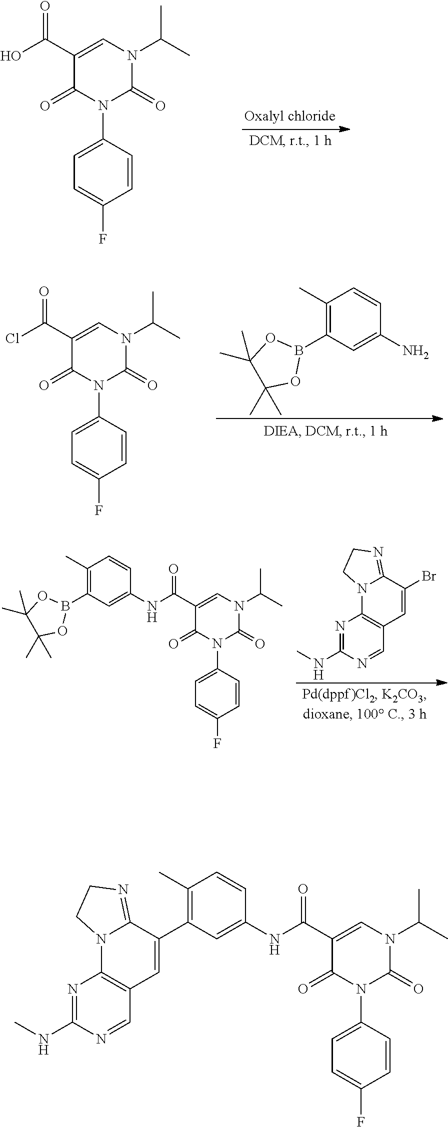

### Step 1: Synthesis of 3-(4-fluorophenyl)-1-isopropyl-2,4-dioxo-1,2,3,4-tetrahydropyrimidine-5-carbonyl chloride

The mixture of 3-(4-fluorophenyl)-1-isopropyl-2,4-dioxo-1,2,3,4-tetrahydropyrimidine-5-carboxylic acid (50 mg, 0.2 mmol, 1.0 eq) in DMF (1 drop) and DCM (2 mL) was added oxalyl chloride (2 mL), the mixture was stirred at r.t. for 0.5 h. The mixture was concentrated in vacuum to give 3-(4-fluorophenyl)-1-isopropyl-2,4-dioxo-1,2,3,4-tetrahydropyrimidine-5-carbonyl chloride (80 mg, crude) as a yellow oil, which was used for the next step directly. LCMS (M+H⁺) m/z: 307.1

### Step 2: Synthesis of 3-(4-fluorophenyl)-1-isopropyl-N-(4-methyl-3-(4,4,5,5-tetramethyl-1,3,2-dioxaborolan-2-yl)phenyl)-2,4-dioxo-1,2,3,4-tetrahydropyrimidine-5-carboxamide

The mixture of 4-methyl-3-(4,4,5,5-tetramethyl-1,3,2-dioxaborolan-2-yl)aniline (50 mg, 0.2 mmol, 1.0 eq) and DIEA (0.5 mL) in DCM (2 mL) was stirred at r.t., Then 3-(4-fluorophenyl)-1-isopropyl-2,4-dioxo-1,2,3,4-tetrahydropyrimidine-5-carbonyl chloride (80 mg crude) in DCM (1 mL) was added. The mixture was stirred r.t. for 1 h. The mixture was concentrated in vacuum to give crude, which was purified on silica gel column flash chromatography (EtOAc:PE=1:3, v/v) to afford 3-(4-fluorophenyl)-1-isopropyl-N-(4-methyl-3-(4,4,5,5-tetramethyl-1,3,2-dioxaborolan-2-yl)phenyl)-2,4-dioxo-1,2,3,4-tetrahydropyrimidine-5-carboxamide (45 mg, 44% yield) as a yellow solid. LCMS (M+H⁺) m/z: 507.9

### Step 3: Synthesis of 3-(4-fluorophenyl)-1-isopropyl-N-(4-methyl-3-(2-(methylamino)-8,9-dihydroimidazo[1′,2′:1,6]pyrido[2,3-d]pyrimidin-6-yl)phenyl)-2,4-dioxo-1,2,3,4-tetrahydropyrimidine-5-carboxamide

The mixture of 3-(4-fluorophenyl)-1-isopropyl-N-(4-methyl-3-(4,4,5,5-tetramethyl-1,3,2-dioxaborolan-2-yl)phenyl)-2,4-dioxo-1,2,3,4-tetrahydropyrimidine-5-carboxamide (45 mg, 0.09 mmol, 1.0 eq), K₂CO₃ (37 mg, 0.27 mmol, 3.0 eq), Pd(dppf)Cl₂ (10 mg, 0.014 mmol) and 6-bromo-N-methyl-8,9-dihydroimidazo[1′,2′:1,6]pyrido[2,3-d]pyrimidin-2-amine (25 mg, 0.09 mmol, 1.0 eq) in dioxane (6 mL) was stirred at 100° C. for 3 h. The mixture was purified by Prep-HPLC (0.1% FA) to afford 3-(4-fluorophenyl)-1-isopropyl-N-(4-methyl-3-(2-(methylamino)-8,9-dihydroimidazo[1′,2′:1,6]pyrido[2,3-d]pyrimidin-6-yl)phenyl)-2,4-dioxo-1,2,3,4-tetrahydropyrimidine-5-carboxamide (8.3 mg, 16% yield) as a brown solid. ¹H NMR (400 MHz, DMSO-d₆): δ 10.85 (s, 1H), 8.62 (s, 1H), 8.35 (s, 1H), 7.60-7.53 (m, 3H), 7.47-7.30 (m, 5H), 7.23-7.22 (m, 1H), 4.80-4.73 (m, 1H), 4.21-4.04 (m, 2H), 3.94-3.89 (m, 2H), 2.87 (s, 3H), 2.18 (s, 3H), 1.41 (d, J=6.8 Hz, 6H). LCMS (M+H⁺) m/z: 581.0.

### Example 194: Preparation of N-(2-fluoro-4-methyl-5-(2-(methylamino)-8,9-dihydroimidazo[1′,2′:1,6]pyrido[2,3-d]pyrimidin-6-yl)phenyl)-3-(4-fluorophenyl)-1-isopropyl-2,4-dioxo-1,2,3,4-tetrahydropyrimidine-5-carboxamide (Compound 194)

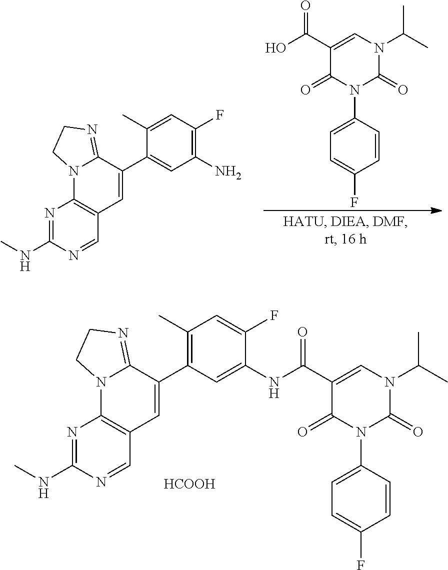 [SMILES: CNc1ncc2c(n1)N1CCN=C1C(c1cc(N)c(F)cc1C)=C2 ; CC(C)n1cc(C(=O)O)c(=O)n(-c2ccc(F)cc2)c1=O ; CNc1ncc2c(n1)N1CCN=C1C(c1cc(NC(=O)c3cn(C(C)C)c(=O)n(-c4ccc(F)cc4)c3=O)c(F)cc1C)=C2 ; "HCOOH"]

The preparation of 6-(5-amino-4-fluoro-2-methylphenyl)-N-methyl-8,9-dihydroimidazo[1′,2′:1,6]pyrido[2,3-d]pyrimidin-2-amine was described in Example 139.

### Step 1: Synthesis of N-(2-fluoro-4-methyl-5-(2-(methylamino)-8,9-dihydroimidazo[1′,2′:1,6]pyrido[2,3-d]pyrimidin-6-yl)phenyl)-3-(4-fluorophenyl)-1-isopropyl-2,4-dioxo-1,2,3,4-tetrahydropyrimidine-5-carboxamide

HATU (86 mg, 0.23 mmol) was added to the mixture of 6-(5-amino-4-fluoro-2-methylphenyl)-N-methyl-8,9-dihydroimidazo[1′,2′:1,6]pyrido[2,3-d]pyrimidin-2-amine (50 mg, 0.15 mmol), 3-(4-fluorophenyl)-1-isopropyl-2,4-dioxo-1,2,3,4-tetrahydropyrimidine-5-carboxylic acid (50 mg, 0.17 mmol), DIEA (58 mg, 0.45 mmol) in DMF (2 mL). The mixture was stirred at rt for 16 hours. The reaction mixture was quenched with water (30 mL) and extracted with DCM (30 mL×2), The combined organic layers were washed with brine (60 mL), dried over Na₂SO₄, filtered, concentrated to afford crude product, which was purified by Prep-HPLC (0.1% HCOOH) to afford N-(2-fluoro-4-methyl-5-(2-(methylamino)-8,9-dihydroimidazo[1′,2′:1,6]pyrido[2,3-d]pyrimidin-6-yl)phenyl)-3-(4-fluorophenyl)-1-isopropyl-2,4-dioxo-1,2,3,4-tetrahydropyrimidine-5-carboxamide formate (23.5 mg, 18% yield) as a yellow solid. ¹H NMR (400 MHz, DMSO-d₆): δ 11.06 (s, 1H), 8.62 (s, 1H), 8.25 (d, J=8.0 Hz, 2H), 8.16 (s, 1H), 7.52-7.41 (m, 3H), 7.37-7.33 (m, 2H), 7.20 (d, J=12.0 Hz, 2H), 4.78-4.74 (m, 1H), 4.08-3.99 (m, 2H), 3.93-3.89 (m, 2H), 2.85 (s, 3H), 2.18 (s, 3H), 1.40 (d, J=6.8 Hz, 6H). LCMS (M+H⁺) m/z: 599.7.

### Example 195: Preparation of N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (Compound 195)

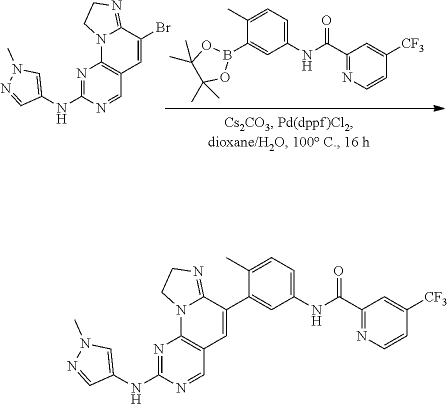

The preparation of 6-bromo-N-(6-methylpyridin-3-yl)-8,9-dihydroimidazo[1′,2′:1,6]pyrido[2,3-d]pyrimidin-2-amine was described in Example 102

### Step 1: Synthesis of N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide

To a solution of 6-bromo-N-(1-methyl-1H-pyrazol-4-yl)-8,9-dihydroimidazo[1′,2′:1,6]pyrido[2,3-d]pyrimidin-2-amine (3.5 g, 10.12 mmol) in dioxane/H₂O (120 mL/20 mL) was added N-(4-methyl-3-(4,4,5,5-tetramethyl-1,3,2-dioxaborolan-2-yl)phenyl)-4-(trifluoromethyl)picolinamide (4.1 g, 10.12 mmol), Pd(dppf)Cl₂ (740 mg, 1.01 mmol), Cs₂CO₃ (9.9 g, 30.35 mmol), the mixture was stirred for 16 h at 100° C. under N₂. The reaction mixture was concentrated and purified by column chromatography (DCM:MeOH=20:1) to afford N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (1.6 g, 29.1% yield) as a yellow solid.

¹H NMR (400 MHz, DMSO-d₆): δ 10.93 (s, 1H), 10.83 (s, 1H), 9.92 (s, 1H), 9.06 (d, J=4.8 Hz, 1H), 9.01 (s, 1H), 8.34 (s, 1H), 8.22 (d, J=6.4 Hz, 1H), 8.12 (d, J=4.4 Hz, 1H), 8.06 (s, 1H), 8.02 (d, J=2.0 Hz, 1H), 7.92 (dd, J=8.4, 2.0 Hz, 1H), 7.72 (s, 1H), 7.42 (d, J=8.4 Hz, 1H), 4.81 (t, J=10.0 Hz, 2H), 4.10-4.06 (m, 2H), 3.90 (s, 3H), 2.23 (s, 3H). LCMS (M+H⁺) m/z: 546.3.

### Example 196: Preparation of 4-(2-fluoropropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 196)

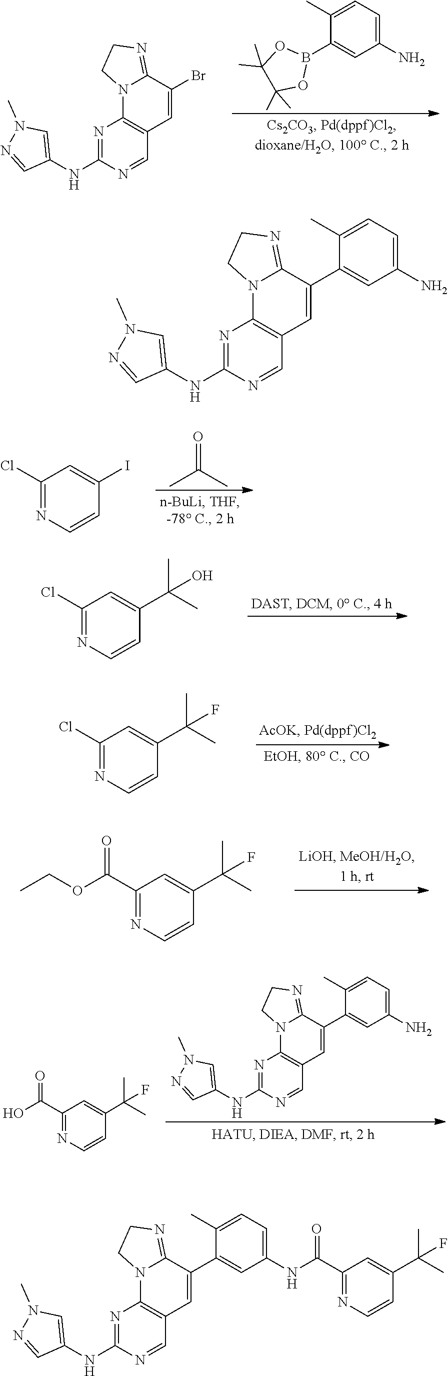

### Step 1: Synthesis of 6-(5-amino-2-methylphenyl)-N-(1-methyl-1H-pyrazol-4-yl)-8,9-dihydroimidazo[1′,2′:1,6]pyrido[2,3-d]pyrimidin-2-amine

A mixture of 6-bromo-N-(1-methyl-1H-pyrazol-4-yl)-8,9-dihydroimidazo[1′,2′:1,6]pyrido[2,3-d]pyrimidin-2-amine (300 mg, 0.87 mmol), 4-methyl-3-(4,4,5,5-tetramethyl-1,3,2-dioxaborolan-2-yl)aniline (202 mg, 0.87 mmol), Cs₂CO₃ (848 mg, 2.60 mmol) and Pd(dppf)Cl₂ (64 mg, 0.087 mmol) in dioxane (6 mL) and water (2 mL) was degassed, charged with N₂ three times and stirred at 100° C. for 2 h. The reaction mixture was concentrated. The residue was purified by column chromatography (DCM/MeOH=20/1, +0.5% TEA) to afford 6-(5-amino-2-methylphenyl)-N-(1-methyl-1H-pyrazol-4-yl)-8,9-dihydroimidazo[1′,2′:1,6]pyrido[2,3-d]pyrimidin-2-amine (200 mg, 62.1% yield) as a yellow solid. LCMS (M+H⁺) m/z: 373.2.

### Step 2: Synthesis of 2-(2-chloropyridin-4-yl)propan-2-ol

A mixture of 2-chloro-4-iodopyridine (500 mg, 2.09 mmol) in dry THE (10 mL) was added n-BuLi (1.3 mL, 1.6 M, 2.09 mmol) at −78° C. and stirred for 15 min, then propan-2-one (364 mg, 6.27 mmol) was added. The mixture was stirred at −78° C. for 2 h. The reaction mixture was quenched with NH₄Cl aq (20 mL) and extracted with EA (20 mL×3), dried over Na₂SO₄, filtered. The combined organic layers were concentrated in vacuum and purified by column chromatography on silica gel (PE/EA=5/1) to afford 2-(2-chloropyridin-4-yl)propan-2-ol (250 mg, 69.7% yield) as white solid. LCMS (M−H)⁻ m/z: 170.2.

### Step 3: Synthesis of 2-chloro-4-(2-fluoropropan-2-yl)pyridine

A mixture of 2-(2-chloropyridin-4-yl)propan-2-ol (240 mg, 1.4 mmol) in dry DCM (10 mL) was cooled to 0° C., and then DAST (560 mg, 3.5 mmol) was added dropwise. The mixture was stirred at rt for 4 h. The reaction mixture was quenched with NaHCO₃ aq (20 mL) and extracted with EA (20 mL×3). The combined organic layers were dried over Na₂SO₄, filtered. Concentration and purification by column chromatography on silica gel (PE/EA=5/1) gave 2-chloro-4-(2-fluoropropan-2-yl)pyridine (130 mg, 53.7% yield) as white solid. LCMS (M+H⁺) m/z: 174.1.

### Step 4: Synthesis of ethyl 4-(2-fluoropropan-2-yl)picolinate

A mixture of 2-chloro-4-(2-fluoropropan-2-yl)pyridine (130 mg, 0.75 mmol), AcOK (293 mg, 3.0 mmol) and Pd(dppf)Cl₂ (27 mg, 0.04 mmol) in EtOH (20 mL) was degassed, charged with CO for three times and stirred at 80° C. for 16 h under CO. The reaction mixture was concentrated and purified by column chromatography on silica gel (PE:EA=4:1) to afford ethyl 4-(2-fluoropropan-2-yl)picolinate (120 mg, 82% yield) as white solid. LCMS (M+H⁺) m/z: 212.1.

### Step 5: Synthesis of 4-(2-fluoropropan-2-yl)picolinic acid

A mixture of ethyl 4-(2-fluoropropan-2-yl)picolinate (120 mg, 0.57 mmol) in MeOH (5 mL) and H₂O (5 mL) was added LiOH (70 mg, 2.84 mmol). The mixture was stirred at r.t for 1 h. The reaction was quenched by NH₄Cl aq. and the aqueous phase was adjusted to pH=2-3 with 1N HCl, and extracted with EA (20 mL×3). The combined organic phase was washed with brine (20 mL), dried over Na₂SO₄, filtered, concentrated and purified by Prep-HPLC (0.1% FA) to afford 4-(2-fluoropropan-2-yl)picolinic acid (80 mg, 77% yield) as a white solid. LCMS (M+H⁺) m/z: 184.0.

### Step 6: Synthesis of 4-(2-fluoropropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (40 mg, 0.11 mmol), 4-(2-fluoropropan-2-yl)picolinic acid (22 mg, 0.12 mmol), HATU (82 mg, 0.22 mmol) and DIEA (42 mg, 0.32 mmol) in dry DMF (5 mL) was stirred at r.t for 2 h. The reaction mixture was diluted with H₂O (20 mL) and extracted with DCM (20 mL×3), dried over Na₂SO₄, filtered. The combined organic layers were concentrated in vacuum and purified by column chromatography on silica gel (DCM/MeOH=10/1) to give crude product, which was purified by Prep-HPLC (0.1% NH₃—H₂O) to afford 4-(2-fluoropropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (17 mg, 26.4% yield) as yellow solid.

¹H NMR (400 MHz, DMSO-d₆): δ 10.64 (s, 1H), 10.07 (br, 1H), 8.74 (d, J=5.2 Hz, 1H), 8.49 (s, 1H), 8.13 (s, 1H), 7.95 (s, 1H), 7.87 (s, 1H), 7.80 (d, J=8.0 Hz, 1H), 7.70 (dd, J=5.2, 1.6 Hz, 1H), 7.60-7.47 (m, 2H), 7.27 (d, J=7.6 Hz, 1H), 4.38-4.33 (m, 2H), 4.00-3.94 (m, 2H), 3.88 (s, 3H), 2.21 (s, 3H), 1.73 (s, 3H), 1.69 (s, 3H). LCMS (M+H⁺) m/z: 538.4.

### Example 197: Preparation of 4-(1,1-difluoroethyl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 197)

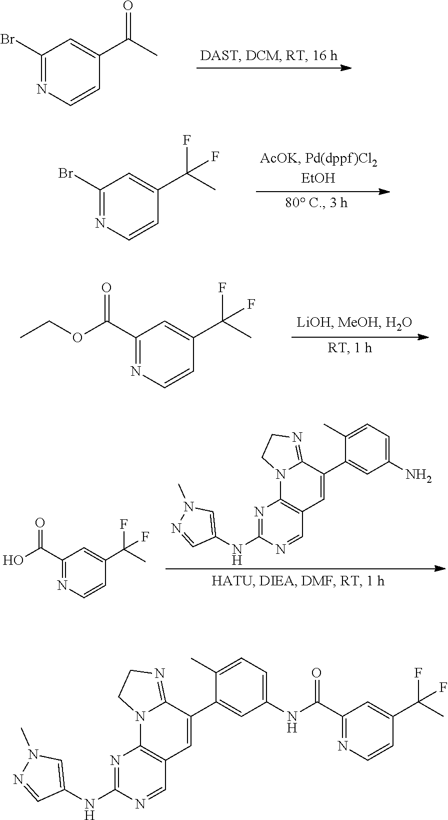

### Step 1: Synthesis of 2-bromo-4-(1,1-difluoroethyl)pyridine

To a solution of 1-(2-bromopyridin-4-yl)ethan-1-one (950 mg, 4.75 mmol) in DCM (10 mL) was added DAST (1.9 g, 11.88 mmol) at 0° C. The reaction mixture was stirred at RT for overnight, quenched with NaHCO₃ (aq.) (20 mL) and extracted with DCM (20 mL×2). The combined organic phase was washed with brine (20 mL), dried with Na₂SO₄, filtered and concentrated in vacuum. The residue was purified by column chromatography on silica gel (PE:EA=5:1) to afford 2-bromo-4-(1,1-difluoroethyl)pyridine (630 mg, 60.3% yield) as a yellow oil.

¹H NMR (400 MHz, DMSO-d₆): δ 8.56 (d, J=5.2 Hz, 1H), 7.85 (s, 1H), 7.65 (d, J=4.8 Hz, 1H), 2.00 (t, J=19.4 Hz, 3H).

### Step 2: Synthesis of ethyl 4-(1,1-difluoroethyl)picolinate

To a solution of 2-bromo-4-(1,1-difluoroethyl)pyridine (630 mg, 2.84 mmol) in EtOH (10 mL) was added AcOK (834 mg, 8.51 mmol) and Pd(dppf)Cl₂ (208 mg, 0.28 mmol). The resulting mixture was stirred at 80° C. for 3 h under CO. The reaction mixture was diluted with water (10 mL) and extracted with EA (10 mL×3). The combined organic phase was washed with brine (10 mL), dried with Na₂SO₄, filtered and concentrated in vacuum. The residue was purified by column chromatography on silica gel (PE:EA=3:1) to afford ethyl 4-(1,1-difluoroethyl)picolinate (600 mg, 98.4% yield) as a yellow oil. LCMS (M+H⁺) m/z: 216.2

### Step 3: Synthesis of 4-(1,1-difluoroethyl)picolinic acid

To a solution of ethyl 4-(1,1-difluoroethyl)picolinate (600 mg, 2.78 mmol) in MeOH/H₂O (10 mL/2 mL) was added LiOH (201 mg, 8.37 mmol). The reaction mixture was stirred for 1 h at RT. The reaction mixture was evaporated to remove MeOH and the aqueous phase was adjusted to pH=2-3 with 1N HCl, and extracted with DCM. The organic layers were combined and dried with Na₂SO₄, filtered and concentrated in vacuum to afford 4-(1,1-difluoroethyl)picolinic acid (440 mg, 84.4% yield) as a white solid. LCMS (M+H⁺) m/z: 188.2.

### Step 4: Synthesis of 4-(1,1-difluoroethyl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (100 mg, 0.27 mmol), 4-(1,1-difluoroethyl)picolinic acid (50 mg, 0.27 mmol), HATU (153 mg, 0.40 mmol) and DIEA (104 mg, 0.81 mmol) in DMF (3 mL) was stirred at RT for 1 h. The reaction mixture was diluted with water (10 mL), the resulting solid was filtered the filtered cake was purified by Prep-HPLC (0.1% NH₃H₂O) to afford 4-(1,1-difluoroethyl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (30.8 mg, 21.2% yield) as a yellow solid.

¹H NMR (400 MHz, DMSO-d₆): δ 10.68 (s, 1H), 9.80 (br, 1H), 8.89 (d, J=4.8 Hz, 1H), 8.32 (s, 1H), 8.22 (d, J=0.8 Hz, 1H), 7.93 (s, 1H), 7.86 (dd, J=4.8, 2.0 Hz, 1H), 7.84 (d, J=2.0 Hz, 1H), 7.78 (dd, J=8.4, 2.0 Hz, 1H), 7.56 (s, 1H), 7.24 (d, J=8.4 Hz, 1H), 7.20 (s, 1H), 4.25-4.19 (m, 2H), 3.97 (t, J=8.8 Hz, 2H), 3.83 (s, 3H), 2.22 (s, 3H), 2.05 (t, J=19.2 Hz, 3H). LCMS (M+H⁺) m/z: 542.4.

### Example 198: Preparation of 4-(tert-butyl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 198)

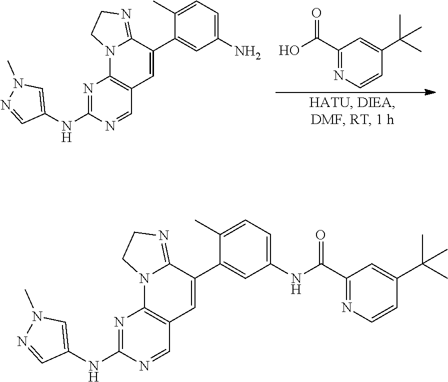 [SMILES: Cc1ccc(N)cc1C1=Cc2cnc(Nc3cnn(C)c3)nc2N2CCN=C12 ; CC(C)(C)c1ccnc(C(=O)O)c1 ; Cc1ccc(NC(=O)c2cc(C(C)(C)C)ccn2)cc1C1=Cc2cnc(Nc3cnn(C)c3)nc2N2CCN=C12]

### Step 1: Synthesis of 4-(tert-butyl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (100 mg, 0.27 mmol), 4-(tert-butyl)picolinic acid (58 mg, 0.27 mmol), HATU (153 mg, 0.40 mmol) and DIEA (104 mg, 0.81 mmol) in DMF (3 mL) was stirred at RT for 1 h. The reaction mixture was diluted with water (10 mL), the resulting solid was filtered and the filtered cake was purified by Prep-HPLC (0.1% NH₃H₂O) to afford 4-(tert-butyl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (30.2 mg, 21.1% yield) as a yellow solid.

¹H NMR (400 MHz, DMSO-d₆): δ 10.56 (s, 1H), 9.78 (s, 1H), 8.64 (d, J=5.2 Hz, 1H), 8.30 (s, 1H), 8.13 (d, J=1.2 Hz, 1H), 7.93 (s, 1H), 7.83 (d, J=2.0 Hz, 1H), 7.76 (dd, J=8.4, 2.0 Hz, 1H), 7.69 (dd, J=5.2, 2.0 Hz, 1H), 7.56 (s, 1H), 7.22 (d, J=8.4 Hz, 1H), 7.18 (s, 1H), 4.21-4.17 (m, 2H), 3.97 (t, J=8.8 Hz, 2H), 3.83 (s, 3H), 2.22 (s, 3H), 1.34 (s, 9H). LCMS (M+H⁺) m/z: 534.4.

### Example 199: Preparation of 4-(2-cyanopropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 199)

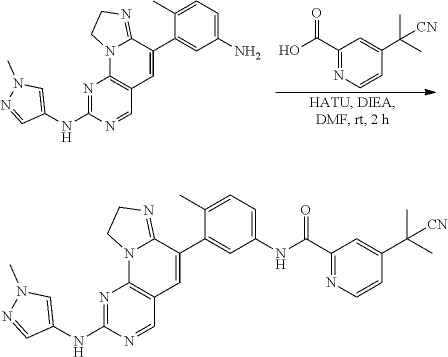 [SMILES: Cc1ccc(N)cc1C1=Cc2cnc(Nc3cnn(C)c3)nc2N2CCN=C12 ; CC(C)(C#N)c1ccnc(C(=O)O)c1 ; Cc1ccc(NC(=O)c2cc(C(C)(C)C#N)ccn2)cc1C1=Cc2cnc(Nc3cnn(C)c3)nc2N2CCN=C12]

### Step 1: Synthesis of 4-(2-cyanopropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (150 mg, 0.40 mmol), 4-(2-cyanopropan-2-yl)picolinic acid (77 mg, 0.40 mmol), HATU (230 mg, 0.60 mmol) and DIEA (156 mg, 1.21 mmol) in DMF (5 mL) was stirred at RT for 2 h. The reaction mixture was diluted with water (20 mL), extracted with EA (50 mL×3). The combined organic phase was washed with brine (50 mL), dried over Na₂SO₄, filtered and concentrated. The residue was purified by Prep-HPLC (0.1% NH₃H₂O) to afford 4-(2-cyanopropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (66.8 mg, 30.5% yield) as a yellow solid.

¹H NMR (400 MHz, DMSO-d₆): δ 10.64 (s, 1H), 9.78 (br, 1H), 9.79 (d, J=5.2 Hz, 1H), 8.30 (s, 1H), 8.26-8.25 (m, 1H), 7.93 (s, 1H), 7.84-7.82 (m, 2H), 7.77 (dd, J=8.4, 2.4 Hz, 1H), 7.56 (s, 1H), 7.23 (d, J=8.4 Hz, 1H), 7.18 (s, 1H), 4.21-4.17 (m, 2H), 3.99-3.95 (m, 2H), 3.82 (s, 3H), 2.22 (s, 3H), 1.76 (s, 6H). LCMS (M+H⁺) m/z: 545.3.

### Example 200: Preparation of 4-(2-hydroxypropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 200)

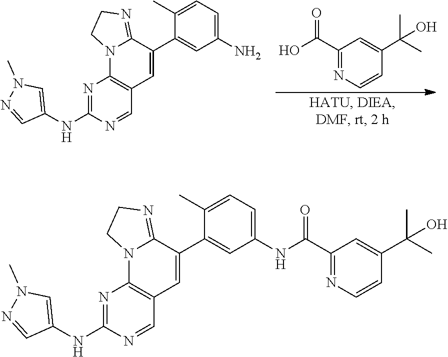 [SMILES: Cc1ccc(N)cc1C1=Cc2cnc(Nc3cnn(C)c3)nc2N2CCN=C12 ; CC(C)(O)c1ccnc(C(=O)O)c1 ; Cc1ccc(NC(=O)c2cc(C(C)(C)O)ccn2)cc1C1=Cc2cnc(Nc3cnn(C)c3)nc2N2CCN=C12]

### Step 1: Synthesis of 4-(2-hydroxypropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (85 mg, 0.23 mmol), 4-(2-hydroxypropan-2-yl)picolinic acid (248 mg (crude), 1.37 mmol), HATU (130 mg, 0.34 mmol) and DIEA (88 mg, 0.69 mmol) in DMF (3 mL) was stirred at RT for 2 h. The reaction mixture was diluted with water (10 mL), extracted with DCM (20 mL×3). The combined organic phase was washed with brine (20 mL), dried over Na₂SO₄, filtered and concentrated. The residue was purified by Prep-HPLC (0.1% NH₃H₂O) to afford 4-(2-hydroxypropan-2-yl)-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (25.5 mg, 20.9% yield) as a yellow solid.

¹H NMR (400 MHz, DMSO-d₆): δ 10.56 (s, 1H), 9.77 (s, 1H), 8.65 (d, J=5.2 Hz, 1H), 8.32 (s, 1H), 8.25 (d, J=1.2 Hz, 1H), 7.93 (s, 1H), 7.83 (d, J=2.4 Hz, 1H), 7.77 (dd, J=8.4, 2.4 Hz, 1H), 7.72 (dd, J=5.2, 1.6 Hz, 1H), 7.56 (s, 1H), 7.23 (d, J=8.4 Hz, 1H), 7.19 (s, 1H), 5.45 (s, 1H), 4.20-4.17 (m, 2H), 4.00-3.96 (m, 2H), 3.83 (s, 3H), 2.22 (s, 3H), 1.47 (s, 6H). LCMS (M+H⁺) m/z: 536.5.

### Example 201: Preparation of N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)-4-(2-hydroxypropan-2-yl)picolinamide (Compound 201)

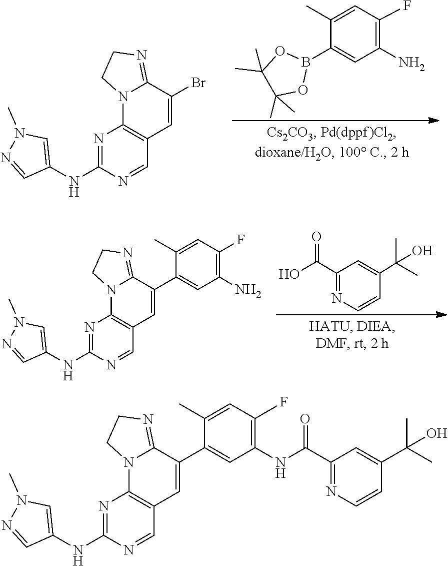 [SMILES: Cn1cc(Nc2ncc3c(n2)N2CCN=C2C(Br)=C3)cn1 ; Cc1cc(F)c(N)cc1B1OC(C)(C)C(C)(C)O1 ; Cc1cc(F)c(N)cc1C1=Cc2cnc(Nc3cnn(C)c3)nc2N2CCN=C12 ; CC(C)(O)c1ccnc(C(=O)O)c1 ; Cc1cc(F)c(NC(=O)c2cc(C(C)(C)O)ccn2)cc1C1=Cc2cnc(Nc3cnn(C)c3)nc2N2CCN=C12]

### Step 1: N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)-4-(2-hydroxypropan-2-yl)picolinamide

A mixture of 6-(5-amino-4-fluoro-2-methylphenyl)-N-(1-methyl-1H-pyrazol-4-yl)-8,9-dihydroimidazo[1′,2′:1,6]pyrido[2,3-d]pyrimidin-2-amine (90 mg, 0.23 mmol), 4-(2-hydroxypropan-2-yl)picolinic acid (250 mg (crude), 1.38 mmol), HATU (131 mg, 0.35 mmol) and DIEA (90 mg, 0.69 mmol) in DMF (3 mL) was stirred at RT for 2 h. The reaction mixture was diluted with water (10 mL), extracted with DCM (20 mL×3). The combined organic phase was washed with brine (20 mL), dried over Na₂SO₄, filtered and concentrated. The residue was purified by Prep-HPLC (0.1% NH₃H₂O) to afford N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)-4-(2-hydroxypropan-2-yl)picolinamide (16.9 mg, 13.2% yield) as a yellow solid.

¹H NMR (400 MHz, DMSO-d₆): δ 10.37 (s, 1H), 9.79 (s, 1H), 8.67 (d, J=4.4 Hz, 1H), 8.31 (s, 1H), 8.26 (s, 1H), 8.01 (d, J=8.0 Hz, 1H), 7.93 (s, 1H), 7.75 (d, J=4.4 Hz, 1H), 7.56 (s, 1H), 7.25 (d, J=12.0 Hz, 1H), 7.20 (s, 1H), 5.48 (s, 1H), 4.18-4.10 (m, 2H), 3.99-3.97 (m, 2H), 3.83 (s, 3H), 2.25 (s, 3H), 1.47 (s, 6H). LCMS (M+H⁺) m/z: 554.2.

### Example 202: Preparation of 4-(2-cyanopropan-2-yl)-N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 202)

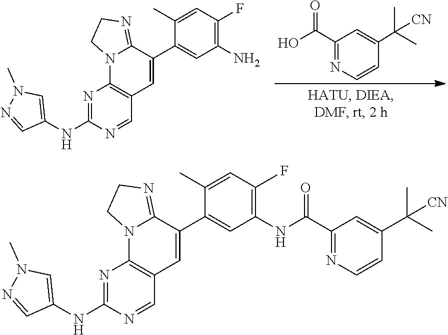 [SMILES: Cc1cc(F)c(N)cc1C1=Cc2cnc(Nc3cnn(C)c3)nc2N2CCN=C12 ; CC(C)(C#N)c1ccnc(C(=O)O)c1 ; Cc1cc(F)c(NC(=O)c2cc(C(C)(C)C#N)ccn2)cc1C1=Cc2cnc(Nc3cnn(C)c3)nc2N2CCN=C12]

### Step 4: Synthesis of 4-(2-cyanopropan-2-yl)-N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-4-fluoro-2-methylphenyl)-N-(1-methyl-1H-pyrazol-4-yl)-8,9-dihydroimidazo[1′,2′:1,6]pyrido[2,3-d]pyrimidin-2-amine (150 mg, 0.38 mmol), 4-(2-cyanopropan-2-yl)picolinic acid (73 mg, 0.38 mmol), HATU (220 mg, 0.58 mmol) and DIEA (149 mg, 1.15 mmol) in DMF (5 mL) was stirred at RT for 2 h. The reaction mixture was diluted with water (20 mL), extracted with EA (50 mL×3). The combined organic phase was washed with brine (50 mL), dried over Na₂SO₄, filtered and concentrated. The residue was purified by Prep-HPLC (0.1% NH₃H₂O) to afford 4-(2-cyanopropan-2-yl)-N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)picolinamide (66.8 mg, 30.5% yield) as a yellow solid.

¹H NMR (400 MHz, DMSO-d₆): δ 10.40 (s, 1H), 9.81 (br, 1H), 8.82 (d, J=5.2 Hz, 1H), 8.30 (s, 1H), 8.26 (d, J=1.2 Hz, 1H), 7.92-7.86 (m, 3H), 7.56-7.54 (m, 1H), 7.27 (d, J=11.6 Hz, 1H), 7.20 (s, 1H), 4.20-4.17 (m, 2H), 3.99-3.95 (m, 2H), 3.82 (s, 3H), 2.25 (s, 3H), 1.76 (s, 6H). LCMS (M+H⁺) m/z: 563.3.

### Example 203: Preparation of N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 203)

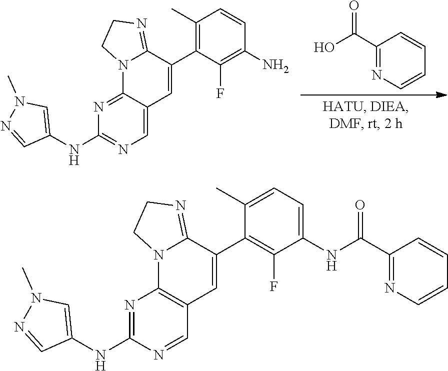 [SMILES: Cc1ccc(N)c(F)c1C1=Cc2cnc(Nc3cnn(C)c3)nc2N2CCN=C12 ; O=C(O)c1ccccn1 ; Cc1ccc(NC(=O)c2ccccn2)c(F)c1C1=Cc2cnc(Nc3cnn(C)c3)nc2N2CCN=C12]

### Step 1: Synthesis of N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

To a solution of 6-(3-amino-2-fluoro-6-methylphenyl)-N-(1-methyl-1H-pyrazol-4-yl)-8,9-dihydroimidazo[1′,2′:1,6]pyrido[2,3-d]pyrimidin-2-amine (30 mg, 0.077 mmol, 1.0 eq) and picolinic acid (14 mg, 0.12 mmol, 1.2 eq) in DMF (5 mL) was added DIEA (0.1 mL) and HATU (58 mg, 0.15 mmol, 2.0 eq). The mixture was stirred at 20° C. under N₂ for 1 h. LCMS showed the reaction was OK. The mixture was concentrated in vacuum and purified by Prep-HPLC (0.1% FA) to afford N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (6.0 mg, 17% yield) as a yellow solid.

¹H NMR (400 MHz, CD₃OD): δ 8.72-8.70 (m, 1H), 8.62-8.59 (m, 1H), 8.30-8.26 (m, 2H), 8.08-8.03 (m, 2H), 7.73-7.63 (m, 3H), 7.26-7.24 (m, 1H), 4.63-4.59 (m, 2H), 4.16-4.12 (m, 2H), 3.94 (s, 3H), 2.31 (s, 3H). LCMS (M+H⁺) m/z: 496.0.

### Example 204: Preparation of 2-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)benzamide (Compound 204)

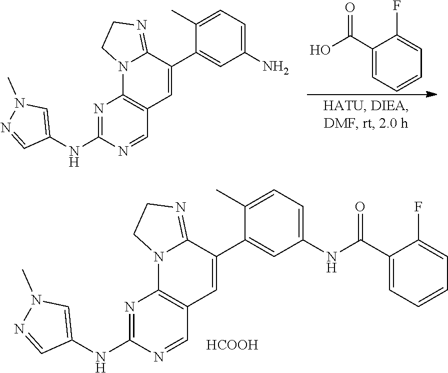

### Step 1: Synthesis of 2-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)benzamide formic acid salt

To a mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (60 mg, 0.16 mmol), 2-fluorobenzoic acid (27 mg, 0.19 mmol) and HATU (92 mg, 0.24 mmol) in DMF (1 mL), was added DIEA (0.1 mL). The resulting mixture was stirred at rt for 2 h. The reaction was purified by Prep-HPLC (0.1% FA) to give 2-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)benzamide formic acid salt (16.5 mg, 21% yield) as a yellow solid.

¹H NMR (400 MHz, DMSO-d₆): δ 10.37 (s, 1H), 9.82-9.67 (m, 1H), 8.32 (s, 1H), 8.23 (s, 2H), 7.92 (s, 1H), 7.64-7.60 (m, 2H), 7.58-7.67 (m, 3H), 7.34-7.33 (m, 2H), 7.22-7.19 (m, 2H), 4.24-4.15 (m, 2H), 3.99-3.97 (m, 2H), 3.82 (s, 3H), 2.20 (s, 3H). LCMS (M+H⁺) m/z: 495.2.

### Example 205: Preparation of 3-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (Compound 205)

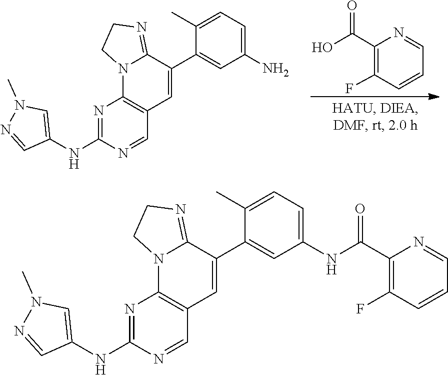 [SMILES: Cc1ccc(N)cc1C1=Cc2cnc(Nc3cnn(C)c3)nc2N2CCN=C12 ; O=C(O)c1ncccc1F ; Cc1ccc(NC(=O)c2ncccc2F)cc1C1=Cc2cnc(Nc3cnn(C)c3)nc2N2CCN=C12]

### Step 1: Synthesis of 3-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide

A mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (60 mg, 0.16 mmol), 3-fluoropicolinic acid (23 mg, 0.16 mmol), HATU (122 mg, 0.32 mmol) and DIEA (62 mg, 0.48 mmol) in dry DMF (3 mL) was stirred at r.t for 2 h. The reaction mixture was diluted with H₂O (20 mL) and extracted with DCM (30 mL×3), dried over Na₂SO₄, filtered. The combined organic layers were concentrated in vacuum and purified by column chromatography on silica gel (DCM/MeOH=15/1) to give crude product, which was purified by Prep-HPLC (0.1% HCOOH) and Prep-HPLC (0.1% NH₃—H₂O) to afford 3-fluoro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)picolinamide (28.1 mg, 35% yield) as yellow solid.

¹H NMR (400 MHz, DMSO-d₆): δ 10.56 (s, 1H), 9.78 (s, 1H), 8.55 (d, J=4.4 Hz, 1H), 8.31 (s, 1H), 7.99-7.80 (m, 2H), 7.76-7.68 (m, 2H), 7.65 (dd, J=8.4, 2.4 Hz, 1H), 7.55 (s, 1H), 7.25-7.15 (m, 2H), 4.19 (s, 2H), 3.98 (t, J=8.4 Hz, 2H), 3.78 (s, 3H), 2.21 (s, 3H). LCMS (M+H⁺) m/z: 496.3.

### Example 206: Preparation of 2-fluoro-N-(4-methyl-3-(2-((4-morpholinophenyl)amino)-8,9-dihydroimidazo[1′,2′:1,6]pyrido[2,3-d]pyrimidin-6-yl)phenyl)benzamide (Compound 206)

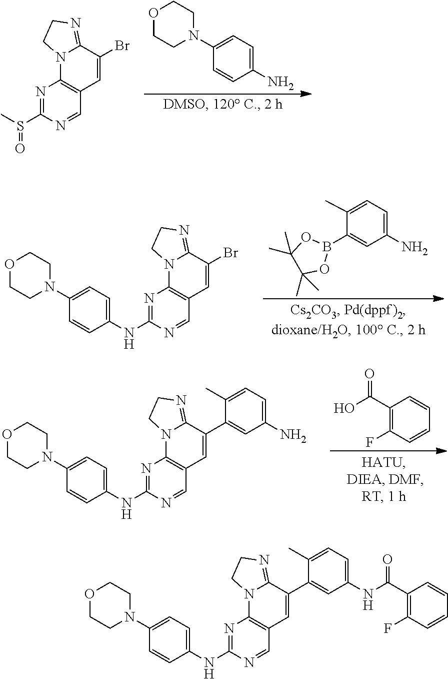

### Step 1: Synthesis of 6-bromo-N-(4-morpholinophenyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (550 mg, 1.76 mmol) in DMSO (10 mL) was added 4-morpholinoaniline (469 mg, 2.64 mmol), the mixture was stirred for 2 h at 120° C. The reaction mixture was poured into water and filtered. and the filter-cake was washed with water, dried in vacuum to afford 6-bromo-N-(4-morpholinophenyl)-8,9-dihydroimidazo[1′,2′:1,6]pyrido[2,3-d]pyrimidin-2-amine (690 mg, 92.0% yield) as a yellow solid. LCMS (M+H⁺) m/z: 426.9 and 428.9

### Step 2: Synthesis of 6-(5-amino-2-methylphenyl)-N-(4-morpholinophenyl)-8,9-dihydroimidazo[1′,2′:1,6]pyrido[2,3-d]pyrimidin-2-amine

To a solution of 6-bromo-N-(4-morpholinophenyl)-8,9-dihydroimidazo[1′,2′:1,6]pyrido[2,3-d]pyrimidin-2-amine (190 mg, 0.44 mmol) in dioxane/H₂O (8 mL/2 mL) was added 4-methyl-3-(4,4,5,5-tetramethyl-1,3,2-dioxaborolan-2-yl)aniline (104 mg, 0.44 mmol), Pd(dppf)Cl₂ (33 mg, 0.044 mmol), Cs₂CO₃ (435 mg, 1.33 mmol). The mixture was stirred for 2 h at 100° C. under N₂. The reaction mixture was filtered and the filtrate was concentrated in vacuum. The residue was purified by column chromatography (DCM:MeOH=20:1) to afford 6-(5-amino-2-methylphenyl)-N-(4-morpholinophenyl)-8,9-dihydroimidazo[1′,2′:1,6]pyrido[2,3-d]pyrimidin-2-amine (200 mg, 99% yield) as a yellow solid. LCMS (M+H⁺) m/z: 454.3.

### Step 3: Synthesis of 2-fluoro-N-(4-methyl-3-(2-((4-morpholinophenyl)amino)-8,9-dihydroimidazo[1′,2′:1,6]pyrido[2,3-d]pyrimidin-6-yl)phenyl)benzamide

To a solution of 6-(5-amino-2-methylphenyl)-N-(4-morpholinophenyl)-8,9-dihydroimidazo[1′,2′:1,6]pyrido[2,3-d]pyrimidin-2-amine (100 mg, 0.22 mmol) in DMF (3 mL) was added 2-fluorobenzoic acid (31 mg, 0.22 mmol), HATU (126 mg, 0.33 mmol), DIPEA (85 mg, 0.66 mmol). The reaction mixture was stirred for 1 h at RT. Water (10 mL) was added to the reaction mixture, the resulting solid was filtered. The filtered cake was purified by prep-HPLC (0.1% NH₃′H₂O) to afford 2-fluoro-N-(4-methyl-3-(2-((4-morpholinophenyl)amino)-8,9-dihydroimidazo[1′,2′:1,6]pyrido[2,3-d]pyrimidin-6-yl)phenyl)benzamide (24.4 mg, 19.2% yield) as a yellow solid.

¹H NMR (400 MHz, DMSO-d₆): δ 10.37 (s, 1H), 9.60 (s, 1H), 8.31 (s, 1H), 7.69-7.64 (m, 4H), 7.58-7.56 (m, 2H), 7.37-7.31 (m, 2H), 7.21 (d, J=8.4 Hz, 1H), 7.17 (s, 1H), 6.91 (d, J=8.8 Hz, 2H), 4.10 (t, J=9.2 Hz, 2H), 3.95 (t, J=9.2 Hz, 2H), 3.74 (s, 4H), 3.04 (s, 4H), 2.20 (s, 3H). LCMS (M+H⁺) m/z: 576.1.

### Example 207: Preparation of N-(4-methyl-3-(2-((6-methylpyridin-3-yl)amino)-8,9-dihydroimidazo[1′,2′:1,6]pyrido[2,3-d]pyrimidin-6-yl)phenyl)-3-(trifluoromethyl)benzamide (Compound 207)

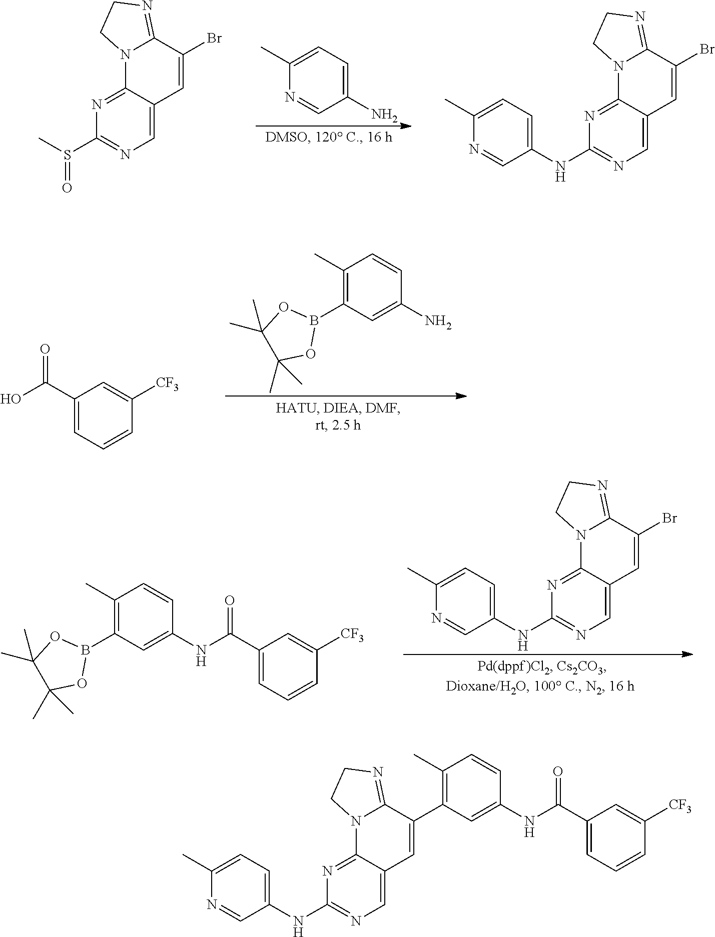

### Step 1: Synthesis of N-(4-methyl-3-(4,4,5,5-tetramethyl-1,3,2-dioxaborolan-2-yl)phenyl)-3-(trifluoromethyl)benzamide

A mixture of 3-(trifluoromethyl)benzoic acid (2.34 g, 12.3 mmol), 4-methyl-3-(4,4,5,5-tetramethyl-1,3,2-dioxaborolan-2-yl)aniline (3.0 g, 12.9 mmol), DIEA (4.6 g, 36.0 mmol) and HATU (7.0 g, 18.0 mmol) in DMF (35.0 mL) was stirred at r.t. for 2.5 h. The reaction mixture was diluted with water (150 mL) and extracted with EA (100 mL×2). The combined organic phase was washed with brine (100 mL), dried over Na₂SO₄, filtered and concentrated. The residue was purified by column chromatography (PE/EA=10/1) to afford N-(4-methyl-3-(4,4,5,5-tetramethyl-1,3,2-dioxaborolan-2-yl)phenyl)-3-(trifluoromethyl)benzamide (4.5 g, 90% yield) as a white solid. LCMS (M+H⁺) m/z: 406.2.

### Step 2: Synthesis of 6-bromo-N-(6-methylpyridin-3-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (3.8 g, 12 mmol) and 6-methylpyridin-3-amine (1.96 g, 18.0 mmol) in DMSO (50.0 mL) was degassed, charged and charged with N₂ three times and stirred at 120° C. for 16 h. The reaction mixture was cooled to r.t., diluted with water (500 mL), stirred at r.t. for 3.0 h, and filtered. The collected cake was dried to afford 6-bromo-N-(6-methylpyridin-3-yl)-8,9-dihydroimidazo[1′,2′:1,6]pyrido[2,3-d]pyrimidin-2-amine (2.76 g, 63.7% yield) as a yellow solid. LCMS (M+H⁺) m/z: 356.9 and 358.9

### Step 3: Synthesis of N-(4-methyl-3-(2-((6-methylpyridin-3-yl)amino)-8,9-dihydroimidazo[1′,2′:1,6]pyrido[2,3-d]pyrimidin-6-yl)phenyl)-3-(trifluoromethyl)benzamide

A mixture of 6-bromo-N-(6-methylpyridin-3-yl)-8,9-dihydroimidazo[1′,2′:1,6]pyrido[2,3-d]pyrimidin-2-amine (1.6 g, 4.48 mmol), N-(4-methyl-3-(4,4,5,5-tetramethyl-1,3,2-dioxaborolan-2-yl)phenyl)-3-(trifluoromethyl)benzamide (1.9 g, 4.7 mmol), Cs₂CO₃ (4.38 g, 13.44 mmol) and Pd(dppf)Cl₂ (328 mg, 0.45 mmol) in dioxane (30.0 mL) and water (3.0 mL) was degassed, charged with N₂ three times and stirred at 100° C. for 16 h. The reaction mixture was cooled to r.t., filtered and concentrated. The residue was purified column chromatography (DCM/MeOH=30/1, +0.5% TEA) to afford crude product, which was triturated with EA (40 mL) and filtered to afford N-(4-methyl-3-(2-((6-methylpyridin-3-yl)amino)-8,9-dihydroimidazo[1′,2′:1,6]pyrido[2,3-d]pyrimidin-6-yl)phenyl)-3-(trifluoromethyl)benzamide (1.155 g, 46% yield) as a yellow solid.

¹H NMR (400 MHz, DMSO-d₆): δ 10.45 (s, 1H), 9.89 (s, 1H), 8.83 (d, J=2.4 Hz, 1H), 8.38 (s, 1H), 8.30 (s, 1H), 8.26 (d, J=7.6 Hz, 1H), 8.12 (dd, J=8.8, 2.4 Hz, 1H), 7.96 (d, J=8.0 Hz, 1H), 7.78 (t, J=7.6 Hz, 1H), 7.69-7.67 (m, 2H), 7.26-7.23 (m, 2H), 7.18 (d, J=8.4 Hz, 1H), 4.13 (t, J=9.6 Hz, 2H), 3.97 (t, J=9.2 Hz, 2H), 2.41 (s, 3H), 2.22 (s, 3H). LCMS (M+H⁺) m/z: 556.5.

### Example 208: Preparation of N-(4-methyl-3-(2-((6-methylpyridin-3-yl)amino)-8,9-dihydroimidazo[1′,2′:1,6]pyrido[2,3-d]pyrimidin-6-yl)phenyl)-4-(trifluoromethyl)picolinamide (Compound 208)

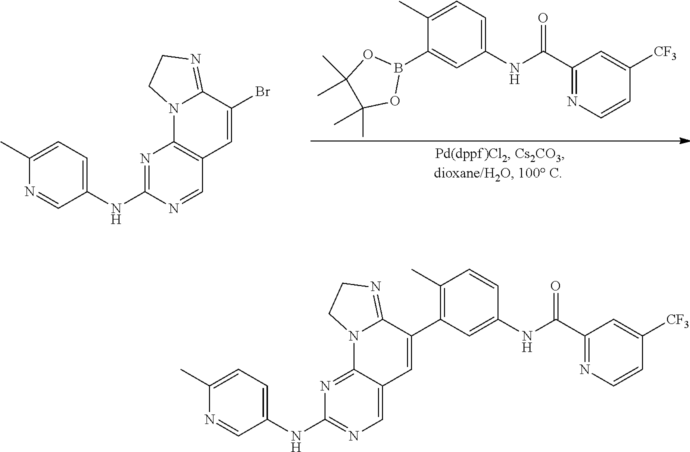 [SMILES: Cc1ccc(Nc2ncc3c(n2)N2CCN=C2C(Br)=C3)cn1 ; Cc1ccc(NC(=O)c2cc(C(F)(F)F)ccn2)cc1B1OC(C)(C)C(C)(C)O1 ; Cc1ccc(Nc2ncc3c(n2)N2CCN=C2C(c2cc(NC(=O)c4cc(C(F)(F)F)ccn4)ccc2C)=C3)cn1]

### Step 1: Synthesis of N-(4-methyl-3-(2-((6-methylpyridin-3-yl)amino)-8,9-dihydroimidazo[1′,2′:1,6]pyrido[2,3-d]pyrimidin-6-yl)phenyl)-4-(trifluoromethyl)picolinamide

To a solution of 6-bromo-N-(6-methylpyridin-3-yl)-8,9-dihydroimidazo[1′,2′:1,6]pyrido[2,3-d]pyrimidin-2-amine (560 mg, 1.56 mmol) in dioxane/H₂O (20 mL/5 mL) was added N-(4-methyl-3-(4,4,5,5-tetramethyl-1,3,2-dioxaborolan-2-yl)phenyl)-4-(trifluoromethyl)picolinamide (635 mg, 1.56 mmol), Pd(dppf)Cl₂ (114 mg, 0.156 mmol), Cs₂CO₃ (1.5 g, 4.68 mmol), the mixture was stirred for 2 h at 120° C. under N₂. The reaction mixture was concentrated and purified by column chromatography (EA:MeOH=10:1) to give N-(4-methyl-3-(2-((6-methylpyridin-3-yl)amino)-8,9-dihydroimidazo[1′,2′:1,6]pyrido[2,3-d]pyrimidin-6-yl)phenyl)-4-(trifluoromethyl)picolinamide (380 mg, 44% yield) as a yellow solid.

¹H NMR (400 MHz, DMSO-d₆): δ 10.77 (s, 1H), 9.90 (s, 1H), 9.03 (d, J=4.8 Hz, 1H), 8.83 (d, J=2.0 Hz, 1H), 8.39 (s, 1H), 8.34 (s, 1H), 8.14-8.08 (m, 2H), 7.82 (d, J=1.6 Hz, 1H), 7.78 (dd, J=8.4, 2.0 Hz, 1H), 7.26-7.24 (m, 2H), 7.19 (d, J=8.4 Hz, 1H), 4.14-4.12 (m, 2H), 4.00-3.97 (m, 2H), 2.42 (s, 3H), 2.22 (s, 3H). LCMS (M+H⁺) m/z: 557.2.

### Example 209: Preparation of N-(4-methyl-3-(2-((2-methylpyridin-3-yl)amino)-8,9-dihydroimidazo[1′,2′:1,6]pyrido[2,3-d]pyrimidin-6-yl)phenyl)-4-(trifluoromethyl)picolinamide (Compound 209)

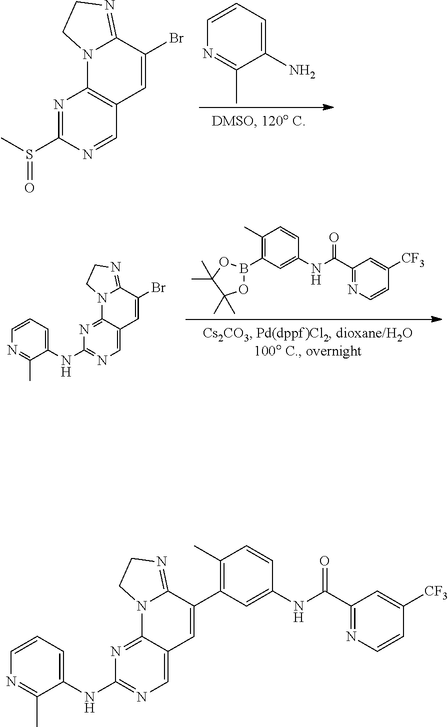

### Step 1: Synthesis of 6-bromo-N-(2-methylpyridin-3-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (106 mg, crude), 2-methylpyridin-3-amine (73 mg, 0.56 mmol) in DMSO (5 mL) was stirred at 120° C. for 16 h. The reaction mixture was purified by Prep-HPLC (0.1% NH₃·H₂O) to afford 6-bromo-N-(2-methylpyridin-3-yl)-8,9-dihydroimidazo[1′,2′:1,6]pyrido[2,3-d]pyrimidin-2-amine (40 mg, 33% yield) as a yellow solid. LCMS (M+H⁺) m/z: 357.1 and 359.1.

### Step 2: Synthesis of N-(4-methyl-3-(2-((2-methylpyridin-3-yl)amino)-8,9-dihydroimidazo[1′,2′:1,6]pyrido[2,3-d]pyrimidin-6-yl)phenyl)-4-(trifluoromethyl)picolinamide formic acid

A mixture of (6-bromo-N-(2-methylpyridin-3-yl)-8,9-dihydroimidazo[1′,2′:1,6]pyrido[2,3-d]pyrimidin-2-amine (40 mg, 0.11 mmol), N-(4-methyl-3-(4,4,5,5-tetramethyl-1,3,2-dioxaborolan-2-yl)phenyl)-4-(trifluoromethyl)picolinamide (55 mg, 0.13 mmol), Cs₂CO₃ (110 mg, 0.34 mmol) and Pd(dppf)Cl₂ (8 mg, 0.01 mmol) in dioxane (10 mL) and water (1 mL) was degassed and charged with N₂ three times and stirred at 100° C. for 16 h. The reaction mixture was concentrated and purified by Prep-HPLC (0.1% FA) to afford N-(4-methyl-3-(2-((2-methylpyridin-3-yl)amino)-8,9-dihydroimidazo[1′,2′:1,6]pyrido[2,3-d]pyrimidin-6-yl)phenyl)-4-(trifluoromethyl)picolinamide formic acid (6.7 mg, 10.7% yield) as a yellow solid.

¹H NMR (400 MHz, DMSO-d₆): δ 10.76 (s, 1H), 9.20 (s, 1H), 9.03 (d, J=5.2 Hz, 1H), 8.33 (s, 1H), 8.30 (s, 1H), 8.23 (dd, J=5.2, 2.0 Hz, 1H), 8.09 (d, J=4.8 Hz, 1H), 7.94 (d, J=7.2 Hz, 1H), 7.83 (d, J=2.4 Hz, 1H), 7.77 (dd, J=8.4, 2.0 Hz, 1H), 7.25-7.21 (m, 2H), 7.19 (s, 1H), 4.02-3.98 (m, 2H), 3.95-3.91 (m, 2H), 2.46 (s, 3H), 2.21 (s, 3H). LCMS (M+H⁺) m/z: 557.5.

### Example 210: Preparation of N-(4-methyl-3-(2-((4-methylpyridin-3-yl)amino)-8,9-dihydroimidazo[1′,2′:1,6]pyrido[2,3-d]pyrimidin-6-yl)phenyl)-4-(trifluoromethyl)picolinamide (Compound 210)

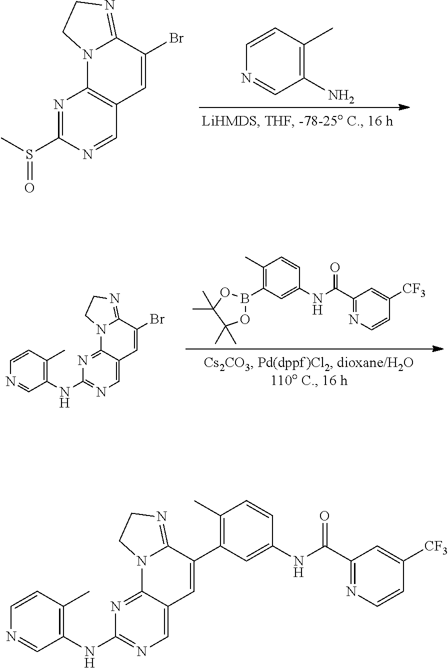

### Step 1: Synthesis of 6-bromo-N-(4-methylpyridin-3-yl)-8,9-dihydroimidazo[1′,2′:1,6]pyrido[2,3-d]pyrimidin-2-amine

LiHMDS (0.7 mL, 0.70 mmol) was added dropwise to the mixture of 4-methylpyridin-3-amine (76 mg, 0.70 mmol) in THF (5 mL) at −60° C. and the mixture was stirred at −60° C. for 0.5 hour. 6-Bromo-2-(methylsulfinyl)-8,9-dihydroimidazo[1′,2′:1,6]pyrido[2,3-d]pyrimidine (200 mg, 0.64 mmol was added to the mixture at −60° C. and the mixture was stirred at r.t for 16 hours. The reaction mixture was quenched with sat NH₄Cl (30 mL) and extracted with DCM (30 mL×3). The combined organic layers were washed with brine (50 mL) and dried over Na₂SO₄, filtered, concentrated to give the crude, which was purified by silica column chromatography (EA:PE=0% to 90% to DCM:MeOH=9:1) to afford 6-bromo-N-(4-methylpyridin-3-yl)-8,9-dihydroimidazo[1′,2′:1,6]pyrido[2,3-d]pyrimidin-2-amine (60 mg, crude) as a yellow oil. LCMS (M+H⁺) m/z: 358.9

### Step 2: Synthesis of N-(4-methyl-3-(2-((4-methylpyridin-3-yl)amino)-8,9-dihydroimidazo[1′,2′:1,6]pyrido[2,3-d]pyrimidin-6-yl)phenyl)-4-(trifluoromethyl)picolinamide

A mixture of 6-bromo-N-(4-methylpyridin-3-yl)-8,9-dihydroimidazo[1′,2′:1,6]pyrido[2,3-d]pyrimidin-2-amine (80 mg, 0.22 mmol), N-(4-methyl-3-(4,4,5,5-tetramethyl-1,3,2-dioxaborolan-2-yl)phenyl)-4-(trifluoromethyl)picolinamide (100 mg, 0.25 mmol), Cs₂CO₃ (215 mg, 0.66 mmol) and Pd(dppf)Cl₂ (16 mg, 0.022 mmol) in dioxane:H₂O (10:1) (5 mL) was degassed and charged with N₂ three times, stirred at 110° C. for 16 hours. The reaction mixture was concentrated and purified by silica column chromatography (DCM:MeOH=10:1) to afford the crude product, which was further purified by trituration with EA:n-hexane (1:10) to afford N-(4-methyl-3-(2-((4-methylpyridin-3-yl)amino)-8,9-dihydroimidazo[1′,2′:1,6]pyrido[2,3-d]pyrimidin-6-yl)phenyl)-4-(trifluoromethyl)picolinamide (9.1 mg, 7.3% yield) as a yellow oil.

¹H NMR (400 MHz, DMSO-d₆): δ 10.79 (s, 1H), 9.39 (s, 1H), 9.04 (d, J=4.8 Hz, 1H), 8.64 (s, 1H), 8.41-8.38 (m, 1H), 8.34 (s, 1H), 8.25 (d, J=4.4 Hz, 1H), 8.10 (d, J=4.0 Hz, 1H), 7.86 (s, 1H), 7.80 (d, J=7.6 Hz, 1H), 7.29-7.26 (m, 3H), 4.11-4.03 (m, 2H), 3.93 (t, J=8.8 Hz, 2H), 2.28 (s, 3H), 2.26 (s, 3H). LCMS (M+H⁺) m/z: 557.1.

### Example 211: Preparation of N-(3-(2-((2,6-dimethylpyridin-3-yl)amino)-8,9-dihydroimidazo[1′,2′:1,6]pyrido[2,3-d]pyrimidin-6-yl)-4-methylphenyl)-4-(trifluoromethyl)picolinamide (Compound 211)

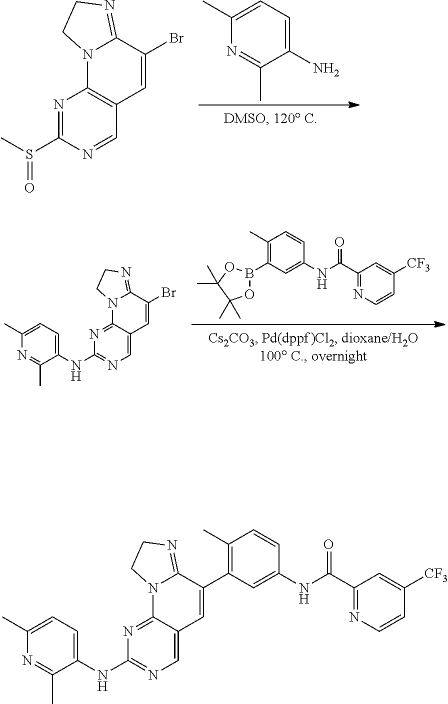

### Step 1: Synthesis of 6-bromo-N-(2,6-dimethylpyridin-3-yl)-8,9-dihydroimidazo[1′,2′:1,6]pyrido[2,3-d]pyrimidin-2-amine

A mixture of 6-bromo-2-(methylsulfinyl)-8,9-dihydroimidazo[1′,2′:1,6]pyrido[2,3-d]pyrimidine (100 mg, crude), 2,6-dimethylpyridin-3-amine (73 mg, 0.56 mmol) in DMSO (5 mL) was stirred at 120° C. for 16 h. The reaction mixture was diluted with DCM (30 mL) and washed with brine (20 mL*2), the combined organic phase was concentrated and purified by silica column chromatography (DCM:MeOH=10:1) and Prep-TLC (PE:EA=0:1) to afford 6-bromo-N-(2,6-dimethylpyridin-3-yl)-8,9-dihydroimidazo[1′,2′:1,6]pyrido[2,3-d]pyrimidin-2-amine (60 mg, 50% yield) as a yellow solid. LCMS (M+H⁺) m/z: 371.0, 373.0.

### Step 2: Synthesis of N-(3-(2-((2,6-dimethylpyridin-3-yl)amino)-8,9-dihydroimidazo[1′,2′:1,6]pyrido[2,3-d]pyrimidin-6-yl)-4-methylphenyl)-4-(trifluoromethyl)picolinamide formic acid

A mixture of 6-bromo-N-(2,6-dimethylpyridin-3-yl)-8,9-dihydroimidazo[1′,2′:1,6]pyrido[2,3-d]pyrimidin-2-amine (60 mg, 0.16 mmol), N-(4-methyl-3-(4,4,5,5-tetramethyl-1,3,2-dioxaborolan-2-yl)phenyl)-4-(trifluoromethyl)picolinamide (79 mg, 0.19 mmol), Cs₂CO₃ (158 mg, 0.49 mmol) and Pd(dppf)Cl₂ (15 mg, 0.05 mmol) in dioxane (10 mL) and water (1 mL) was degassed and charged with N₂ three times and stirred at 100° C. for 16 h. The reaction mixture was concentrated and purified by silica column chromatography (DCM:MeOH=10:1) first and then by Prep-HPLC (0.1% FA) to afford N-(3-(2-((2,6-dimethylpyridin-3-yl)amino)-8,9-dihydroimidazo[1′,2′:1,6]pyrido[2,3-d]pyrimidin-6-yl)-4-methylphenyl)-4-(trifluoromethyl)picolinamide formic acid (10.0 mg, 10.9% yield) as a yellow solid.

¹H NMR (400 MHz, DMSO-d₆): δ 10.75 (s, 1H), 9.14 (s, 1H), 9.03 (d, J=4.8 Hz, 1H), 8.33 (s, 1H), 8.28 (s, 1H), 8.20 (s, 1H), 8.09 (d, J=4.8 Hz, 1H), 7.83 (d, J=2.0 Hz, 1H), 7.78-7.74 (m, 2H), 7.24 (d, J=8.4 Hz, 1H), 7.19 (s, 1H), 7.07 (d, J=8.4 Hz, 1H), 4.02-3.98 (m, 2H), 3.93-3.88 (m, 2H), 2.41 (s, 3H), 2.39 (s, 3H), 2.21 (s, 3H). LCMS (M+H⁺) m/z: 571.2.

### Example 212: Preparation of N-(4-methyl-3-(2-((2-methylpyridin-4-yl)amino)-8,9-dihydroimidazo[1′,2′:1,6]pyrido[2,3-d]pyrimidin-6-yl)phenyl)-4-(trifluoromethyl)picolinamide (Compound 212)

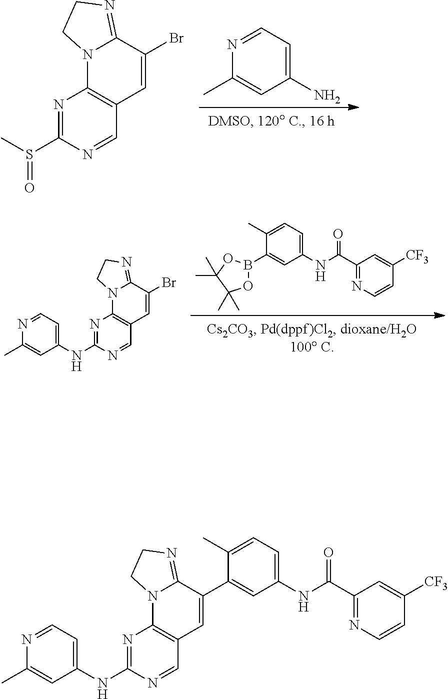

### Step 1: Synthesis of 6-bromo-N-(2-methylpyridin-4-yl)-8,9-dihydroimidazo[1′,2′:1,6]pyrido[2,3-d]pyrimidin-2-amine

To a mixture of 6-bromo-2-(methylsulfinyl)-8,9-dihydroimidazo[1′,2′:1,6]pyrido[2,3-d]pyrimidine (200 mg, crude) in DMSO (5 mL) was added 2-methylpyridin-3-amine (41 mg, 0.10 mmol), the mixture was stirred at 120° C. for 16 h. The mixture was diluted with aq. NH₄Cl (20 mL) and extracted with DCM (30 mL×2). The combined organic phase was concentrated and purified by Prep-TLC (DCM:MeOH=10:1) to afford 6-bromo-N-(2-methylpyridin-4-yl)-8,9-dihydroimidazo[1′,2′:1,6]pyrido[2,3-d]pyrimidin-2-amine (31 mg, 13.6% yield) as a yellow solid. LCMS (M+H⁺) m/z: 357.1 and 359.1.

### Step 2: Synthesis of N-(4-methyl-3-(2-((2-methylpyridin-4-yl)amino)-8,9-dihydroimidazo[1′,2′:1,6]pyrido[2,3-d]pyrimidin-6-yl)phenyl)-4-(trifluoromethyl)picolinamide

A mixture of 6-bromo-N-(2-methylpyridin-4-yl)-8,9-dihydroimidazo[1′,2′:1,6]pyrido[2,3-d]pyrimidin-2-amine (30 mg, 0.08 mmol), N-(4-methyl-3-(4,4,5,5-tetramethyl-1,3,2-dioxaborolan-2-yl)phenyl)-4-(trifluoromethyl)picolinamide (41 mg, 0.10 mmol), Cs₂CO₃ (82 mg, 0.25 mmol) and Pd(dppf)Cl₂ (6 mg, 0.05 mmol) in dioxane (10 mL) and water (1 mL) was degassed and charged with N₂ three times, stirred at 100° C. for 16 h. The reaction mixture was concentrated and purified by silica column chromatography (DCM:MeOH=10:1) first and then by Prep-HPLC (0.1% FA) to afford N-(4-methyl-3-(2-((2-methylpyridin-4-yl)amino)-8,9-dihydroimidazo[1′,2′:1,6]pyrido[2,3-d]pyrimidin-6-yl)phenyl)-4-(trifluoromethyl)picolinamide (2.3 mg, 4.9% yield) as a yellow solid.

¹H NMR (400 MHz, DMSO-d₆): δ 10.78 (s, 1H), 10.18 (s, 1H), 9.03 (d, J=5.2 Hz, 1H), 8.47 (s, 1H), 8.33 (s, 1H), 8.25 (d, J=5.6 Hz, 1H), 8.09 (d, J=4.0 Hz, 1H), 7.87 (d, J=2.0 Hz, 1H), 7.79 (dd, J=8.4, 2.0 Hz, 1H), 7.70 (s, 1H), 7.66 (d, J=6.0 Hz, 1H), 7.29-7.25 (m, 2H), 4.22-4.18 (m, 2H), 4.03-3.98 (m, 2H), 2.42 (s, 3H), 2.22 (s, 3H). LCMS (M+H⁺) m/z: 557.8.

### Example 213: Preparation of N-(4-methyl-3-(2-((3-methylpyridin-4-yl)amino)-8,9-dihydroimidazo[1′,2′:1,6]pyrido[2,3-d]pyrimidin-6-yl)phenyl)-4-(trifluoromethyl)picolinamide (Compound 213)

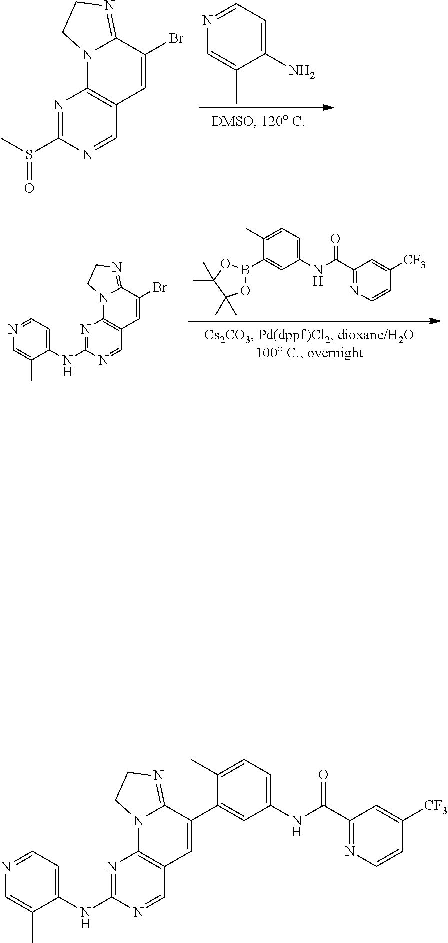

### Step 1: Synthesis of 6-bromo-N-(3-methylpyridin-4-yl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (80 mg, 0.26 mmol) in DMSO (2 mL) was added 3-methylpyridin-4-amine (33.5 mg, 0.310 mmol). The mixture was stirred at 120° C. for 16 hours. The reaction mixture was purified by Prep-HPLC (0.1% NH₃·H₂O) to afford 6-bromo-N-(3-methylpyridin-4-yl)-8,9-dihydroimidazo[1′,2′:1,6]pyrido[2,3-d]pyrimidin-2-amine (30 mg, 26% yield) as a yellow solid. LCMS (M+H⁺) m/z: 357.1.

### Step 2: Synthesis of N-(4-methyl-3-(2-((3-methylpyridin-4-yl)amino)-8,9-dihydroimidazo[1′,2′:1,6]pyrido[2,3-d]pyrimidin-6-yl)phenyl)-4-(trifluoromethyl)picolinamide

A mixture of 6-bromo-N-(3-methylpyridin-4-yl)-8,9-dihydroimidazo[1′,2′:1,6]pyrido[2,3-d]pyrimidin-2-amine (20 mg, 0.056 mmol), N-(4-methyl-3-(4,4,5,5-tetramethyl-1,3,2-dioxaborolan-2-yl)phenyl)-4-(trifluoromethyl)picolinamide (25 mg, 0.062 mmol), Cs₂CO₃ (54.6 mg, 0.17 mmol) and Pd(dppf)Cl₂ (4.1 mg, 0.0056 mmol) in dioxane (1.8 mL) and water (0.2 mL) was degassed and charged with N₂ three times, stirred at 110° C. for 2 hours. The reaction mixture was concentrated and purified by silica column (DCM:MeOH=10:1), followed by Prep-HPLC (0.1% FA) and by Prep-TLC (DCM:MeOH=10:1) to afford N-(4-methyl-3-(2-((3-methylpyridin-4-yl)amino)-8,9-dihydroimidazo[1′,2′:1,6]pyrido[2,3-d]pyrimidin-6-yl)phenyl)-4-(trifluoromethyl)picolinamide (2.9 mg, 6% yield) as a yellow solid.

¹H NMR (400 MHz, DMSO-d₆): δ 10.79 (s, 1H), 9.15 (br, 1H), 9.03 (d, J=5.2 Hz, 1H), 8.48 (s, 1H), 8.33-8.31 (m, 3H), 8.10 (d, J=6.0 Hz, 1H), 8.02 (d, J=5.6 Hz, 1H), 7.88 (d, J=2.0 Hz, 1H), 7.79 (dd, J=8.4, 2.4 Hz, 1H), 7.35 (s, 1H), 7.27 (d, J=8.4 Hz, 1H), 4.16-4.14 (m, 2H), 3.97 (t, J=9.6 Hz, 2H), 2.30 (s, 3H), 2.23 (s, 3H). LCMS (M+H⁺) m/z: 557.8.

### Example 214: Preparation of N-(4-methyl-3-(2-(pyridin-4-ylamino)-8,9-dihydroimidazo[1′,2′:1,6]pyrido[2,3-d]pyrimidin-6-yl)phenyl)-4-(trifluoromethyl)picolinamide (Compound 214)

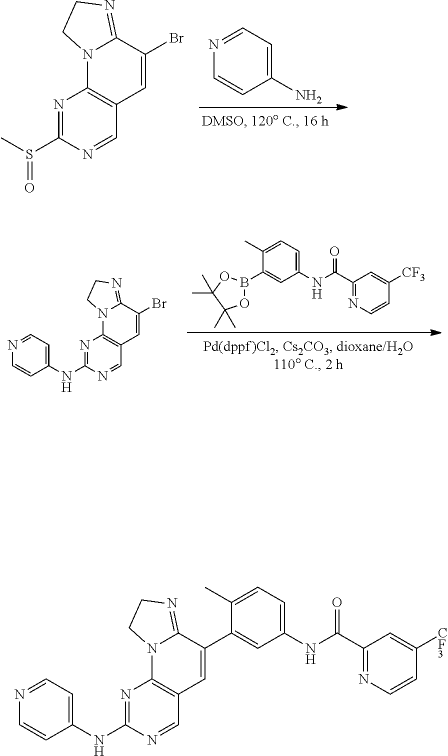

### Step 1: Synthesis of 6-bromo-N-(pyridin-4-yl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (100 mg, 0.32 mmol) in DMSO (2 mL) was added pyridin-4-amine (36 mg, 0.38 mmol). The mixture was stirred at 120° C. for 16 hours. The reaction mixture was purified by Prep-HPLC (0.1% NH₃·H₂O) to afford 6-bromo-N-(pyridin-4-yl)-8,9-dihydroimidazo[1′,2′:1,6]pyrido[2,3-d]pyrimidin-2-amine (40 mg, 36% yield) as a yellow solid. LCMS (M+H⁺) m/z: 343.1.

### Step 2: Synthesis of N-(4-methyl-3-(2-(pyridin-4-ylamino)-8,9-dihydroimidazo[1′,2′:1,6]pyrido[2,3-d]pyrimidin-6-yl)phenyl)-4-(trifluoromethyl)picolinamide

A mixture of 6-bromo-N-(pyridin-4-yl)-8,9-dihydroimidazo[1′,2′:1,6]pyrido[2,3-d]pyrimidin-2-amine (30 mg, 0.087 mmol), N-(4-methyl-3-(4,4,5,5-tetramethyl-1,3,2-dioxaborolan-2-yl)phenyl)-4-(trifluoromethyl)picolinamide (39 mg, 0.096 mmol), Cs₂CO₃ (85 mg, 0.26 mmol) and Pd(dppf)Cl₂ (6.4 mg, 0.0087 mmol) in dioxane (4 mL) and water (0.5 mL) was degassed and charged with N₂ three times, stirred at 110° C. for 2 hours. The reaction mixture was concentrated and purified by silica column chromatography (EA/PE 5% to 90% DCM:MeOH=10:1) to give the crude product, which was triturated with MeOH (5 mL) to afford N-(4-methyl-3-(2-(pyridin-4-ylamino)-8,9-dihydroimidazo[1′,2′:1,6]pyrido[2,3-d]pyrimidin-6-yl)phenyl)-4-(trifluoromethyl)picolinamide (13.4 mg, 21% yield) as a yellow solid.

¹H NMR (400 MHz, DMSO-d₆): δ 10.78 (s, 1H), 10.24 (s, 1H), 9.03 (d, J=4.4 Hz, 1H), 8.45 (s, 1H), 8.38 (d, J=5.2 Hz, 2H), 8.34 (s, 1H), 8.09 (d, J=3.2 Hz, 1H), 7.87-7.79 (m, 4H), 7.27-7.25 (m, 2H), 4.19 (t, J=9.2 Hz, 2H), 4.00 (t, J=9.2 Hz, 2H), 2.23 (s, 3H). LCMS (M+H⁺) m/z: 543.4.

### Example 215: Preparation of N-(4-methyl-3-(2-(pyridin-3-ylamino)-8,9-dihydroimidazo[1′,2′:1,6]pyrido[2,3-d]pyrimidin-6-yl)phenyl)-4-(trifluoromethyl)picolinamide (Compound 215)

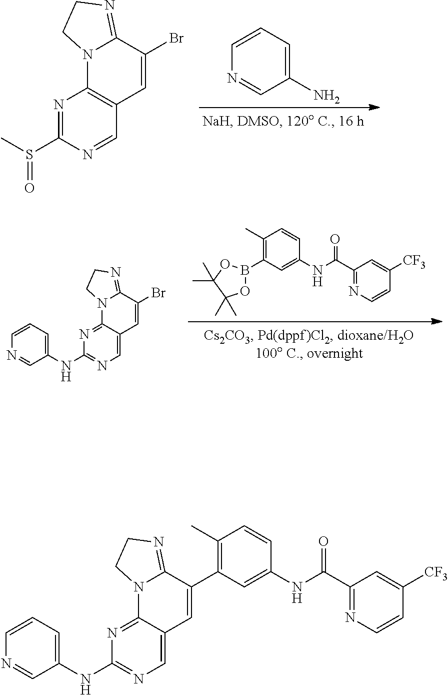

### Step 1: Synthesis of 6-bromo-N-(2-methylpyridin-3-yl)-8,9-dihydroimidazo[1′,2′:1,6]pyrido[2,3-d]pyrimidin-2-amine

A mixture of pyridin-3-amine (30 mg, 0.32 mmol) in DMF (5 mL) was added NaH (16 mg, 0.38 mmol) at 0° C. and stirred for 0.5 h, then 6-bromo-2-(methylsulfinyl)-8,9-dihydroimidazo[1′,2′:1,6]pyrido[2,3-d]pyrimidine (106 mg, crude) was added, the mixture was stirred at rt for 16 h. The reaction mixture was concentrated and the residue was purified on silica gel chromatography (DCM/MeOH=10:1) to afford 6-bromo-N-(2-methylpyridin-3-yl)-8,9-dihydroimidazo[1′,2′:1,6]pyrido[2,3-d]pyrimidin-2-amine (40 mg, 33% yield) as a yellow solid. LCMS (M+H⁺) m/z: 343.0, 345.0.

### Step 2: Synthesis of N-(4-methyl-3-(2-(pyridin-3-ylamino)-8,9-dihydroimidazo[1′,2′:1,6]pyrido[2,3-d]pyrimidin-6-yl)phenyl)-4-(trifluoromethyl)picolinamide

A mixture of (6-bromo-N-(2-methylpyridin-3-yl)-8,9-dihydroimidazo[1′,2′:1,6]pyrido[2,3-d]pyrimidin-2-amine (40 mg, 0.12 mmol), N-(4-methyl-3-(4,4,5,5-tetramethyl-1,3,2-dioxaborolan-2-yl)phenyl)-4-(trifluoromethyl)picolinamide (57 mg, 0.14 mmol), Cs₂CO₃ (114 mg, 0.35 mmol) and Pd(dppf)Cl₂ (8 mg, 0.01 mmol) in dioxane (10 mL) and water (1 mL) was degassed and charged with N₂ three times and stirred at 100° C. for 16 h. The reaction mixture was concentrated and purified by Prep-HPLC (0.1% FA) to afford N-(4-methyl-3-(2-(pyridin-3-ylamino)-8,9-dihydroimidazo[1′,2′:1,6]pyrido[2,3-d]pyrimidin-6-yl)phenyl)-4-(trifluoromethyl)picolinamide (2.8 mg, 4.4% yield) as a yellow solid.

¹H NMR (400 MHz, DMSO-d₆): δ 10.77 (s, 1H), 10.00 (s, 1H), 9.03 (d, J=4.8 Hz, 1H), 8.95 (d, J=2.8 Hz, 1H), 8.40 (s, 1H), 8.34 (s, 1H), 8.31-8.25 (m, 2H), 8.18 (dd, J=4.8, 1.2 Hz, 1H), 8.08 (dd, J=5.2, 1.2 Hz, 1H), 7.86 (d, J=2.4 Hz, 1H), 7.78 (dd, J=8.4, 2.4 Hz, 1H), 7.35-7.32 (m, 1H), 7.26-7.23 (m, 2H), 4.14 (t, J=9.6 Hz, 2H), 3.98 (t, J=9.6 Hz, 2H), 2.23 (s, 3H). LCMS (M+H⁺) m/z: 543.4.

### Example 216: Preparation of N-(4-methyl-3-(2-(pyridin-2-ylamino)-8,9-dihydroimidazo[1′,2′:1,6]pyrido[2,3-d]pyrimidin-6-yl)phenyl)-4-(trifluoromethyl)picolinamide (Compound 216)

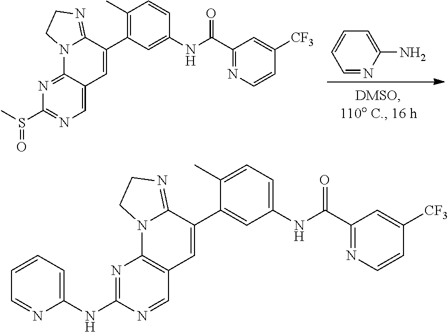 [SMILES: Cc1ccc(NC(=O)c2cc(C(F)(F)F)ccn2)cc1C1=Cc2cnc(S(C)=O)nc2N2CCN=C12 ; Nc1ccccn1 ; Cc1ccc(NC(=O)c2cc(C(F)(F)F)ccn2)cc1C1=Cc2cnc(Nc3ccccn3)nc2N2CCN=C12]

The preparation of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide was described in Example 106

### Step 1: Synthesis of N-(4-methyl-3-(2-(pyridin-2-ylamino)-8,9-dihydroimidazo[1′,2′:1,6]pyrido[2,3-d]pyrimidin-6-yl)phenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (120 mg, crude) and pyridin-2-amine (100 mg) in DMSO (0.5 mL) was stirred at 110° C. for 16 h. Water was added, the reaction mixture was extracted with EA. The combined extracts were washed with brine. Concentration and purification by flash (DCM:MeOH=10:1) and Prep-HPLC (NH₄HCO₃) gave N-(4-methyl-3-(2-(pyridin-2-ylamino)-8,9-dihydroimidazo[1′,2′:1,6]pyrido[2,3-d]pyrimidin-6-yl)phenyl)-4-(trifluoromethyl)picolinamide (5.0 mg) as yellow solid.

¹H NMR (400 MHz, DMSO-d₆): δ 10.80 (s, 1H), 9.92 (s, 1H), 9.03 (d, J=5.2 Hz, 1H), 8.42 (s, 1H), 8.38 (d, J=8.0 Hz, 1H), 8.34 (s, 1H), 8.30 (d, J=1.2 Hz, 1H), 8.10 (d, J=5.2 Hz, 1H), 7.88-7.86 (m, 1H), 7.80-7.77 (m, 2H), 7.26-7.25 (m, 2H), 7.04-7.02 (m, 1H), 4.19-4.14 (m, 2H), 3.99-3.95 (m, 2H), 2.23 (s, 3H). LCMS (M+H⁺) m/z: 543.0.

### Example 217: Preparation of N-(4-methyl-3-(2-(pyrazin-2-ylamino)-8,9-dihydroimidazo[1′,2′:1,6]pyrido[2,3-d]pyrimidin-6-yl)phenyl)-4-(trifluoromethyl)picolinamide (Compound 217)

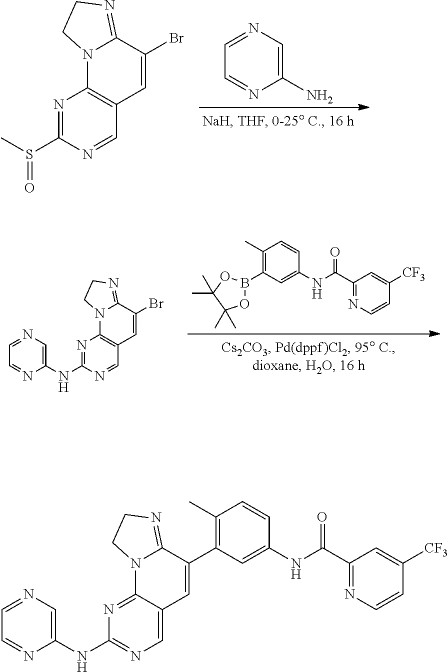

### Step 1: Synthesis of 6-bromo-N-(pyrazin-2-yl)-8,9-dihydroimidazo[1′,2′:1,6]pyrido[2,3-d]pyrimidin-2-amine

To a solution of pyrazin-2-amine (61 mg 0.64 mmol) in dry THF (5 mL) was added NaH (32 mg, 0.80 mmol) at 0° C. The mixture was stirred at 0° C. for 1 h, then 6-bromo-2-(methylsulfinyl)-8,9-dihydroimidazo[1′,2′:1,6]pyrido[2,3-d]pyrimidine (100 mg, 0.32 mmol) was added. The mixture was stirred at r.t. for 16 h. The reaction mixture was quenched with NH₄Cl aq. and diluted with EA. The combined extracts were concentrated in vacuum and purified by column chromatography on silica gel (DCM/MeOH=10/1) to afford 6-bromo-N-(pyrazin-2-yl)-8,9-dihydroimidazo[1′,2′:1,6]pyrido[2,3-d]pyrimidin-2-amine (35 mg, 31.8% yield) as brown solid. LCMS (M+H⁺) m/z: 344.1 and 346.1.

### Step 2: Synthesis of N-(4-methyl-3-(2-(pyrazin-2-ylamino)-8,9-dihydroimidazo[1′,2′:1,6]pyrido[2,3-d]pyrimidin-6-yl)phenyl)-4-(trifluoromethyl)picolinamide

A mixture of 6-bromo-N-(pyrazin-2-yl)-8,9-dihydroimidazo[1′,2′:1,6]pyrido[2,3-d]pyrimidin-2-amine (45 mg, 0.131 mmol), N-(4-methyl-3-(4,4,5,5-tetramethyl-1,3,2-dioxaborolan-2-yl)phenyl)-4-(trifluoromethyl)picolinamide (58 mg, 0.144 mmol), Cs₂CO₃ (128 mg, 0.392 mmol) and Pd(dppf)Cl₂ (5 mg, 0.006 mmol) in dioxane (5 mL) and water (0.5 mL) was degassed, charged with N₂ three times and stirred at 95° C. for 16 h. The reaction mixture was concentrated in vacuum and purified by column chromatography on silica gel (DCM/MeOH=10/1) to give crude product, which was triturated with MeOH (1 mL) to afford N-(4-methyl-3-(2-(pyrazin-2-ylamino)-8,9-dihydroimidazo[1′,2′:1,6]pyrido[2,3-d]pyrimidin-6-yl)phenyl)-4-(trifluoromethyl)picolinamide (15.3 mg, 13.1% yield) as a yellow solid.

¹H NMR (400 MHz, DMSO-d₆): δ 10.83 (s, 1H), 10.74 (s, 1H), 9.60 (s, 1H), 9.04 (d, J=4.8 Hz, 1H), 8.74 (s, 1H), 8.41 (s, 1H), 8.34-8.32 (m, 2H), 8.10 (d, J=4.8 Hz, 1H), 7.94 (s, 1H), 7.86-7.83 (m, 1H), 7.69-7.65 (m, 1H), 7.33 (d, J=8.4 Hz, 1H), 4.42-4.40 (m, 2H), 4.03 (t, J=9.6 Hz, 2H), 2.23 (s, 3H). LCMS (M+H⁺) m/z: 544.3.

### Example 218: Preparation of N-(3-(2-((3-chloropyridin-2-yl)amino)-8,9-dihydroimidazo[1′,2′:1,6]pyrido[2,3-d]pyrimidin-6-yl)-4-methylphenyl)-4-(trifluoromethyl)picolinamide (Compound 218)

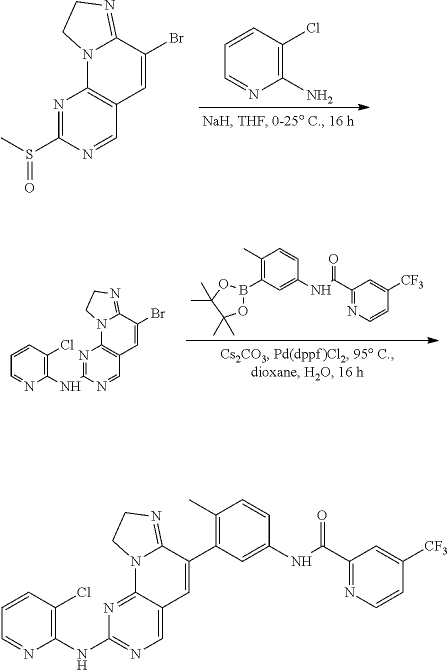

### Step 1: Synthesis of 6-bromo-N-(3-chloropyridin-2-yl)-8,9-dihydroimidazo[1′,2′:1,6]pyrido[2,3-d]pyrimidin-2-amine

To a solution of 3-chloropyridin-2-amine (83 mg 0.64 mmol) in dry THF (5 mL) was added NaH (32 mg, 0.80 mmol) at 0° C. and the mixture was stirred at 0° C. for 1 h, then 6-bromo-2-(methylsulfinyl)-8,9-dihydroimidazo[1′,2′:1,6]pyrido[2,3-d]pyrimidine (100 mg, 0.32 mmol) was added. The mixture was stirred at r.t. for 16 h. The reaction mixture was concentrated in vacuum and purified by column chromatography on silica gel (DCM/MeOH=15/1) to give crude product, which was purified by Prep-HPLC (0.1% NH₃—H₂O) to afford 6-bromo-N-(3-chloropyridin-2-yl)-8,9-dihydroimidazo[1′,2′:1,6]pyrido[2,3-d]pyrimidin-2-amine (20 mg, 16.6% yield) as brown solid. LCMS (M+H⁺) m/z: 377.0 and 379.0.

### Step 2: Synthesis of N-(3-(2-((3-chloropyridin-2-yl)amino)-8,9-dihydroimidazo[1′,2′:1,6]pyrido[2,3-d]pyrimidin-6-yl)-4-methylphenyl)-4-(trifluoromethyl)picolinamide formic acid

A mixture of 6-bromo-N-(pyrazin-2-yl)-8,9-dihydroimidazo[1′,2′:1,6]pyrido[2,3-d]pyrimidin-2-amine (48 mg, 0.127 mmol), N-(4-methyl-3-(4,4,5,5-tetramethyl-1,3,2-dioxaborolan-2-yl)phenyl)-4-(trifluoromethyl)picolinamide (57 mg, 0.140 mmol), Cs₂CO₃ (125 mg, 0.381 mmol) and Pd(dppf)Cl₂ (5 mg, 0.006 mmol) in dioxane (5 mL) and water (0.5 mL) was degassed and charged with N₂ three times and stirred at 95° C. for 16 h. The reaction mixture was concentrated in vacuum and purified by column chromatography on silica gel (DCM/MeOH=10/1) to give crude product, which was purified by Prep-HPLC (0.1% FA) to afford N-(3-(2-((3-chloropyridin-2-yl)amino)-8,9-dihydroimidazo[1′,2′:1,6]pyrido[2,3-d]pyrimidin-6-yl)-4-methylphenyl)-4-(trifluoromethyl)picolinamide formic acid (17.3 mg, 21. % yield) as a yellow solid.

¹H NMR (400 MHz, DMSO-d₆): δ 10.76 (s, 1H), 9.87 (s, 1H), 9.03 (d, J=4.8 Hz, 1H), 8.38 (dd, J=4.4, 1.2 Hz, 1H), 8.33 (s, 1H), 8.29 (s, 1H), 8.20 (s, 1H), 8.09 (d, J=4.8 Hz, 1H), 7.98 (dd, J=8.0, 1.2 Hz, 1H), 7.83 (d, J=2.4 Hz, 1H), 7.78 (dd, J=8.4, 2.0 Hz, 1H), 7.31 (dd, J=8.0, 4.8 Hz, 1H), 7.24 (d, J=8.4 Hz, 1H), 7.20 (s, 1H), 3.99-3.90 (m, 4H), 2.21 (s, 3H). LCMS (M+H⁺) m/z: 577.3.

### Example 219: Preparation of N-(3-(2-((3-fluoropyridin-4-yl)amino)-8,9-dihydroimidazo[1′,2′:1,6]pyrido[2,3-d]pyrimidin-6-yl)-4-methylphenyl)-4-(trifluoromethyl)picolinamide (Compound 219)

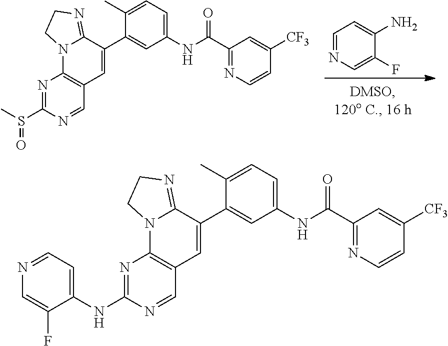 [SMILES: Cc1ccc(NC(=O)c2cc(C(F)(F)F)ccn2)cc1C1=Cc2cnc(S(C)=O)nc2N2CCN=C12 ; Nc1ccncc1F ; Cc1ccc(NC(=O)c2cc(C(F)(F)F)ccn2)cc1C1=Cc2cnc(Nc3ccncc3F)nc2N2CCN=C12]

The preparation of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide was described in Example 106

### Step 1: Synthesis of N-(3-(2-((3-fluoropyridin-4-yl)amino)-8,9-dihydroimidazo[1′,2′:1,6]pyrido[2,3-d]pyrimidin-6-yl)-4-methylphenyl)-4-(trifluoromethyl)picolinamide

A mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (130 mg, 0.25 mmol), 3-fluoropyridin-4-amine (280 mg, 2.54 mmol) in DMSO (6.0 mL) was degassed, charged with N₂ three times and stirred at 120° C. for 16 h. The reaction mixture was cooled to r.t., diluted with water (20 mL) and extracted with EA (50 mL×3). The combined organic phase was washed with brine (50 mL), dried over Na₂SO₄, filtered and concentrated. The residue was purified by Prep-HPLC (0.1% NH₃H₂O) to afford N-(3-(2-((3-fluoropyridin-4-yl)amino)-8,9-dihydroimidazo[1′,2′:1,6]pyrido[2,3-d]pyrimidin-6-yl)-4-methylphenyl)-4-(trifluoromethyl)picolinamide (5 mg, 4% yield) as a yellow solid.

¹H NMR (400 MHz, CD₃OD): δ 9.73 (br, 1H), 9.55-9.49 (m, 2H), 8.98 (br, 1H), 8.49-8.43 (m, 2H), 8.00-7.89 (m, 3H), 7.50 (d, J=8.0 Hz, 1H), 7.33 (s, 1H), 5.08-4.94 (m, 2H), 4.40-4.37 (m, 2H), 2.35 (s, 3H). LCMS (M+H⁺) m/z: 561.1.

### Example 220: Preparation of N-(3-(2-((3-fluoropyridin-2-yl)amino)-8,9-dihydroimidazo[1′,2′:1,6]pyrido[2,3-d]pyrimidin-6-yl)-4-methylphenyl)-4-(trifluoromethyl)picolinamide (Compound 220)

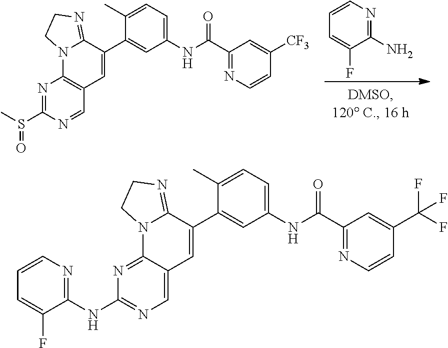

### Step 1: Synthesis of N-(3-(2-((3-fluoropyridin-4-yl)amino)-8,9-dihydroimidazo[1′,2′:1,6]pyrido[2,3-d]pyrimidin-6-yl)-4-methylphenyl)-4-(trifluoromethyl)picolinamide

A mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (100 mg, 0.20 mmol), 3-fluoropyridin-2-amine (219 mg, 2.0 mmol) in DMSO (3.0 mL) was degassed, charged with N₂ three times and stirred at 120° C. for 16 h. The reaction mixture was cooled to r.t., diluted with water (20 mL) and extracted with EA (50 mL×3). The combined organic phase was washed with brine (100 mL), dried over Na₂SO₄, filtered and concentrated. The residue was purified by Prep-HPLC (0.1% NH₃H₂O) to afford N-(3-(2-((3-fluoropyridin-4-yl)amino)-8,9-dihydroimidazo[1′,2′:1,6]pyrido[2,3-d]pyrimidin-6-yl)-4-methylphenyl)-4-(trifluoromethyl)picolinamide (15 mg, 14% yield) as a yellow solid.

¹H NMR (400 MHz, DMSO-d₆): δ 10.77 (s, 1H), 9.96 (s, 1H), 9.03 (d, J=5.2 Hz, 1H), 8.34 (s, 1H), 8.30 (s, 1H), 8.23 (d, J=4.4 Hz, 1H), 8.08 (d, J=4.8 Hz, 1H), 7.84 (d, J=2.0 Hz, 1H), 7.79-7.72 (m, 2H), 7.34-7.30 (m, 1H), 7.24 (d, J=8.4 Hz, 1H), 7.21 (s, 1H), 3.99-3.92 (m, 4H), 2.22 (s, 3H). LCMS (M+H⁺) m/z: 561.1.

### Example 221: Preparation of N-(3-(2-((6-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (Compound 221)

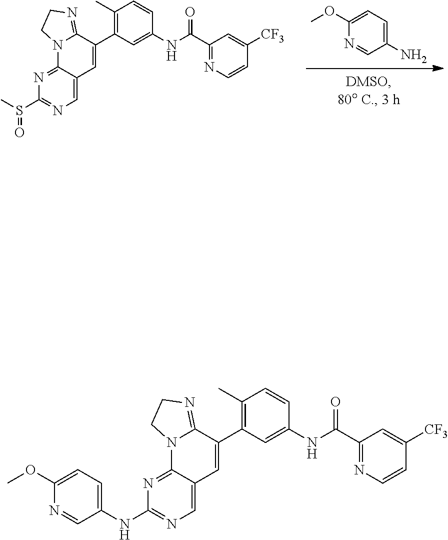

### Step 1: Synthesis of N-(3-(2-((6-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide

To a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (70 mg, crude) in DMSO (8 drops) was added 6-methoxypyridin-3-amine (124 mg, 1 mmol). The mixture was stirred at 80° C. for 3 h. LCMS showed the reaction completed. The mixture was diluted with water (10 mL), extracted by EA (10 mL×3). The combined organic phase was washed by brine (20 mL), dried over anhydrous Na₂SO₄, filtered and concentrated to afford a crude solid, which was purified by prep-HPLC to afford N-(3-(2-((6-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (18 mg) as yellow solid.

¹H NMR (400 MHz, DMSO-d₆): δ 10.79 (s, 1H), 9.79 (s, 1H), 9.03 (d, J=5.2 Hz, 1H), 8.60 (s, 1H), 8.36-8.33 (m, 2H), 8.09-8.06 (m, 2H), 7.86 (d, J=2.0 Hz, 1H), 7.78 (dd, J=8.4, 2.0 Hz, 1H), 7.28-7.22 (m, 2H), 6.81 (d, J=9.2 Hz, 1H), 4.12 (t, J=9.6 Hz, 2H), 3.96 (t, J=9.6 Hz, 2H), 3.83 (s, 3H), 2.22 (s, 3H). LCMS (M+H⁺) m/z: 573.0.

### Example 222: Preparation of N-(3-(2-((2-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (Compound 222)

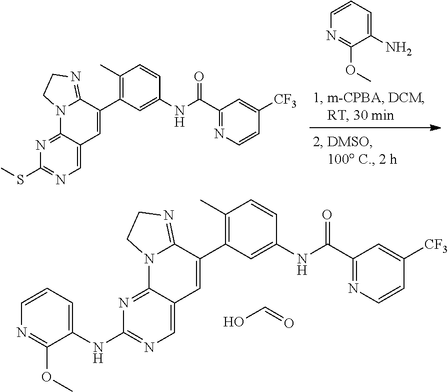

### Step 1: Synthesis of N-(3-(2-((2-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide

To a mixture of N-(4-methyl-3-(2-(methylthio)-8,9-dihydroimidazo[1′,2′:1,6]pyrido[2,3-d]pyrimidin-6-yl)phenyl)-4-(trifluoromethyl)picolinamide (50 mg, 0.1 mmol) in DCM (3 mL), was added m-CPBA (43 mg, 0.25 mmol). The mixture was stirred for 0.5 h at 25° C. The reaction was concentrated and solution of 2-methoxypyridin-3-amine (62 mg, 0.5 mmol) in DMSO (0.1 mL) was added to reaction mixture. The reaction was stirred at 100° C. for 2 h and purified by Prep-HPLC (0.5% FA) to give N-(3-(2-((2-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (4.8 mg, 8% yield) as yellow solid.

¹H NMR (400 MHz, CD₃OD): δ 8.98 (d, J=5.2 Hz, 1H), 8.77 (dd, J=8.0, 1.2 Hz, 1H), 8.51 (s, 1H), 8.44 (s, 1H), 7.94 (d, J=4.4 Hz, 1H), 7.85-7.82 (m, 2H), 7.76-7.75 (m, 1H), 7.47 (s, 1H), 7.35 (d, J=8.4 Hz, 1H), 7.04-7.01 (m, 1H), 4.64 (s, 1H), 4.42 (t, J=9.6 Hz, 2H), 4.12-4.09 (m, 2H), 4.08 (s, 3H), 2.30 (s, 3H). LCMS (M+H⁺) m/z: 573.0.

### Example 223 and Example 224: Preparation of N-(3-(2-((4-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (Compound 223) and Preparation of N-(3-(2-((4-hydroxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (Compound 224)

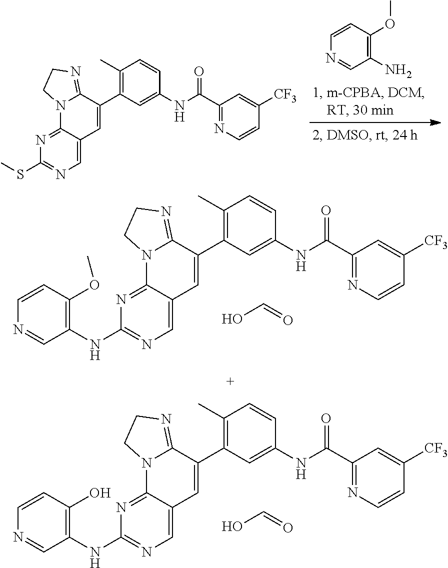

### Step 1: Synthesis of N-(3-(2-((4-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide and N-(3-(2-((4-hydroxypyridin-3-yl)amino)-8,9-dihydroimidazo[1′,2′:1,6]pyrido[2,3-d]pyrimidin-6-yl)-4-methylphenyl)-4-(trifluoromethyl)picolinamide

To a mixture of N-(4-methyl-3-(2-(methylthio)-8,9-dihydroimidazo[1′,2′:1,6]pyrido[2,3-d]pyrimidin-6-yl)phenyl)-4-(trifluoromethyl)picolinamide (50 mg, 0.1 mmol), was added m-CPBA (43 mg, 0.25 mmol). The mixture was stirred for 0.5 h at 25° C. The reaction was concentrated and a solution of 4-methoxypyridin-3-amine (62 mg, 0.5 mmol) in DMSO (0.5 mL) was added to reaction mixture. The reaction was stirred at 25° C. for 16 h and purified by Prep-HPLC(NH4HCO3) to give N-(3-(2-((4-methoxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (3.6 mg) as yellow solid. And N-(3-(2-((4-hydroxypyridin-3-yl)amino)-8,9-dihydroimidazo[1′,2′:1,6]pyrido[2,3-d]pyrimidin-6-yl)-4-methylphenyl)-4-(trifluoromethyl)picolinamide (2.5 mg) as yellow solid.

Compound 223:1H NMR (400 MHz, CD₃OD): δ 9.43 (dd, J=7.6, 2.0 Hz, 1H), 9.33 (d, J=2.0 Hz, 1H), 8.99 (d, J=5.2 Hz, 1H), 8.77 (s, 1H), 8.50 (br, 1H), 8.44 (s, 1H), 7.95 (d, J=4.0 Hz, 1H), 7.91 (d, J=2.4 Hz, 1H), 7.75 (dd, J=8.4, 2.4 Hz, 1H), 7.60 (d, J=7.2 Hz, 1H), 7.53 (s, 1H), 7.38 (d, J=8.4 Hz, 1H), 4.43 (t, J=9.6 Hz, 2H), 4.32 (s, 3H), 4.19 (t, J=9.6 Hz, 2H), 2.32 (s, 3H). LCMS (M+H⁺) m/z: 573.0.

Compound 224: ¹H NMR (400 MHz, CD₃OD): δ 8.99-8.97 (m, 2H), 8.75 (d, J=2.0 Hz, 1H), 8.66 (s, 1H), 8.44 (s, 1H), 7.94 (d, J=5.2 Hz, 1H), 7.86 (d, J=2.0 Hz, 1H), 7.77 (dd, J=8.0, 2.0 Hz, 1H), 7.47 (s, 1H), 7.36 (d, J=8.0 Hz, 1H), 6.48 (d, J=8.0 Hz, 1H), 4.41 (t, J=9.6 Hz, 2H), 4.14 (t, J=9.6 Hz, 2H), 2.32 (s, 3H). LCMS (M+H⁺) m/z: 559.0.

### Example 225: Preparation of N-(3-(2-((2-fluorophenyl)amino)-8,9-dihydroimidazo[1′,2′:1,6]pyrido[2,3-d]pyrimidin-6-yl)-4-methylphenyl)-4-(trifluoromethyl)picolinamide (Compound 225)

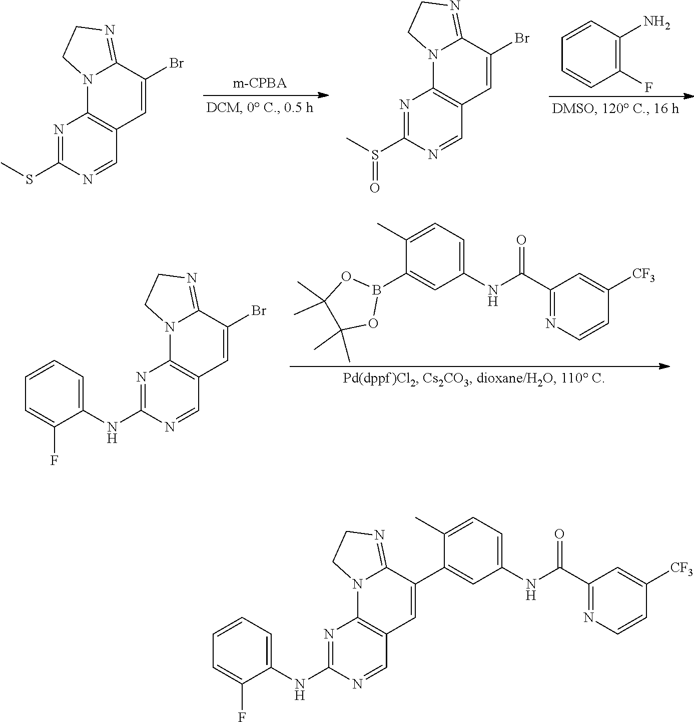

### Step 1: Synthesis of 6-bromo-N-(2-fluorophenyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (100 mg, 0.32 mmol) in DMSO (5 mL) was added 2-fluoroaniline (71 mg, 0.72 mmol). The reaction mixture was stirred for 16 h at 120° C. The reaction mixture was diluted with EA (30 mL), washed with water (10 mL×3), brine (10 mL×3), dried over Na₂SO₄, concentrated under vacuum. The residue was triturated with EA (8 mL) and filtered to afford 6-bromo-N-(2-fluorophenyl)-8,9-dihydroimidazo[1′,2′:1,6]pyrido[2,3-d]pyrimidin-2-amine (33 mg, 29% yield) as a yellow solid. LCMS (M+H⁺) m/z: 361.9.

### Step 2: Synthesis of N-(3-(2-((2-fluorophenyl)amino)-8,9-dihydroimidazo[1′,2′:1,6]pyrido[2,3-d]pyrimidin-6-yl)-4-methylphenyl)-4-(trifluoromethyl)picolinamide

To a mixture of 6-bromo-N-(2-fluorophenyl)-8,9-dihydroimidazo[1′,2′:1,6]pyrido[2,3-d]pyrimidin-2-amine (33 mg, 0.091 mmol) in dioxane/H₂O (6 mL/2 mL) was added N-(4-methyl-3-(4,4,5,5-tetramethyl-1,3,2-dioxaborolan-2-yl)phenyl)-4-(trifluoromethyl)picolinamide (39 mg, 0.096 mmol), Pd(dppf)Cl₂ (6.7 mg, 0.009 mmol), Cs₂CO₃ (89 mg, 0.27 mmol). The mixture was stirred for 1.5 h at 110° C. under N₂. The reaction mixture was diluted with EA (25 mL), then washed with H₂O (10 mL×3), brine (10 mL×3). The organic layer was dried over Na₂SO₄, concentrated under vacuum. The residue was purified by column chromatography (EA=100%) to afford N-(3-(2-((2-fluorophenyl)amino)-8,9-dihydroimidazo[1′,2′:1,6]pyrido[2,3-d]pyrimidin-6-yl)-4-methylphenyl)-4-(trifluoromethyl)picolinamide (22.2 mg, 44% yield) as a light yellow solid.

¹H NMR (400 MHz, DMSO-d₆): δ 10.76 (s, 1H), 9.27 (s, 1H), 9.03 (d, J=5.2 Hz, 1H), 8.33 (s, 1H), 8.32 (s, 1H), 8.09-8.08 (m, 1H), 7.84-7.84 (m, 2H), 7.79-7.64 (m, 1H), 7.25-7.16 (m, 5H), 4.06-4.02 (m, 2H), 3.95-3.90 (m, 2H), 2.22 (s, 3H). LCMS (M+H⁺) m/z: 560.4.

### Example 226: Preparation of N-(3-(2-((3-fluorophenyl)amino)-8,9-dihydroimidazo[1′,2′:1,6]pyrido[2,3-d]pyrimidin-6-yl)-4-methylphenyl)-4-(trifluoromethyl)picolinamide (Compound 226)

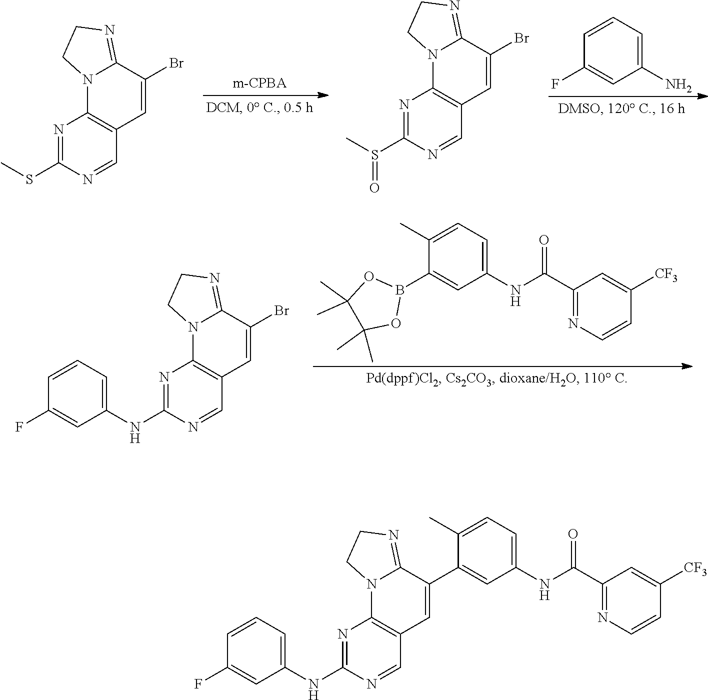

### Step 1: Synthesis of 6-bromo-N-(3-fluorophenyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (100 mg, 0.32 mmol) in DMSO (5 mL) was added 3-fluoroaniline (53 mg, 0.48 mmol). The reaction mixture was stirred for 16 h at 120° C. The reaction mixture was diluted with EA (30 mL). The organic was washed with aq. NH₄Cl (10 mL×3) and brine (10 mL×3), dried over Na₂SO₄, and then concentrated under vacuum. The residue was triturated with EA (5 mL) and filtered to afford 6-bromo-N-(3-fluorophenyl)-8,9-dihydroimidazo[1′,2′:1,6]pyrido[2,3-d]pyrimidin-2-amine (53 mg, 46% yield) as a brown solid. LCMS (M+H⁺) m/z: 359.9 and 361.9.

### Step 2: Synthesis of N-(3-(2-((3-fluorophenyl)amino)-8,9-dihydroimidazo[1′,2′:1,6]pyrido[2,3-d]pyrimidin-6-yl)-4-methylphenyl)-4-(trifluoromethyl)picolinamide

To a solution of 6-bromo-N-(3-fluorophenyl)-8,9-dihydroimidazo[1′,2′:1,6]pyrido[2,3-d]pyrimidin-2-amine (53 mg, 0.14 mmol) in dioxane/H₂O (6 mL/2 mL) was added N-(4-methyl-3-(4,4,5,5-tetramethyl-1,3,2-dioxaborolan-2-yl)phenyl)-4-(trifluoromethyl)picolinamide (63 mg, 0.15 mmol), Pd(dppf)Cl₂ (11 mg, 0.014 mmol), Cs₂CO₃ (144 mg, 0.44 mmol), the mixture was stirred for 1.5 h at 110° C. under N₂. The reaction mixture was diluted with EA (30 mL). The organic was washed with H₂O (10 mL×2) and brine (10 mL×2), dried over Na₂SO₄, concentrated under vacuum. The residue was purified by column chromatography (EA=100%), followed by trituration with EA (5 mL) to afford N-(3-(2-((3-fluorophenyl)amino)-8,9-dihydroimidazo[1′,2′:1,6]pyrido[2,3-d]pyrimidin-6-yl)-4-methylphenyl)-4-(trifluoromethyl)picolinamide (8.6 mg, 11% yield) as a yellow solid.

¹H NMR (400 MHz, DMSO-d₆): δ 10.77 (s, 1H), 10.05 (s, 1H), 9.03 (d, J=4.8 Hz, 1H), 8.41 (s, 1H), 8.34 (s, 1H), 8.09 (d, J=3.6 Hz, 1H), 7.91-7.86 (m, 2H), 7.78 (d, J=8.8 Hz, 1H), 7.56 (d, J=8.0 Hz, 1H), 7.33-7.31 (m, 1H), 7.26-7.24 (m, 2H), 6.80-6.76 (m, 1H), 4.18-4.12 (m, 2H), 4.00-3.96 (m, 2H), 2.23 (s, 3H). LCMS (M+H⁺) m/z: 560.4.

### Example 227: Preparation of N-(3-(2-((4-fluorophenyl)amino)-8,9-dihydroimidazo[1′,2′:1,6]pyrido[2,3-d]pyrimidin-6-yl)-4-methylphenyl)-4-(trifluoromethyl)picolinamide (Compound 227)

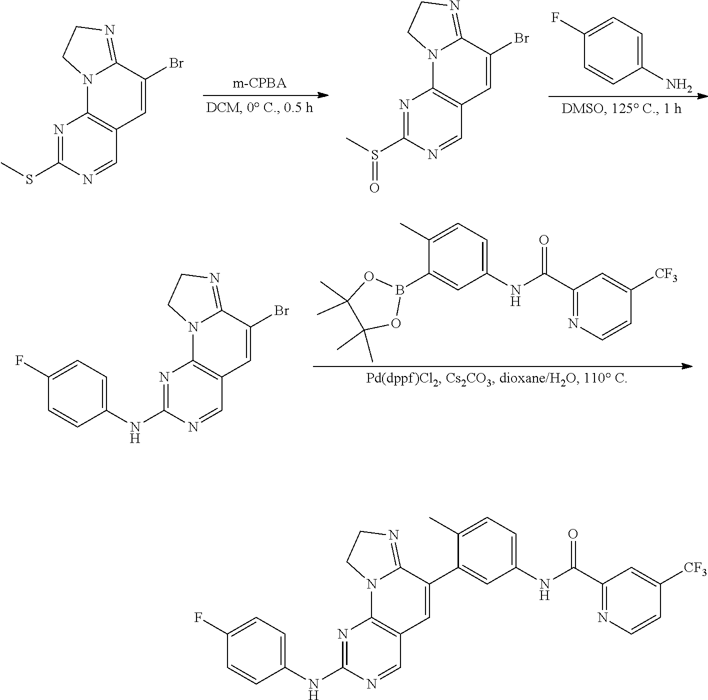

### Step 1: Synthesis of 6-bromo-N-(4-fluorophenyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (113 mg, 0.36 mmol) in DMSO (5 mL) was added 4-fluoroaniline (80 mg, 0.72 mmol). The reaction mixture was stirred for 1 h at 125° C. The reaction mixture was diluted with EA (30 mL). The organic layer was washed with water (10 mL×3), brine (10 mL×3), dried over Na₂SO₄, concentrated under vacuum. The residue was triturated with EA (10 mL) and filtered to afford 6-bromo-N-(4-fluorophenyl)-8,9-dihydroimidazo[1′,2′:1,6]pyrido[2,3-d]pyrimidin-2-amine (67 mg, 51% yield) as a yellow solid. LCMS (M+H⁺) m/z: 360.0 and 362.0.

### Step 2: Synthesis of N-(3-(2-((4-fluorophenyl)amino)-8,9-dihydroimidazo[1′,2′:1,6]pyrido[2,3-d]pyrimidin-6-yl)-4-methylphenyl)-4-(trifluoromethyl)picolinamide

A mixture of 6-bromo-N-(4-fluorophenyl)-8,9-dihydroimidazo[1′,2′:1,6]pyrido[2,3-d]pyrimidin-2-amine (67 mg, 0.186 mmol) in dioxane/H₂O (7 mL/3 mL) was added N-(4-methyl-3-(4,4,5,5-tetramethyl-1,3,2-dioxaborolan-2-yl)phenyl)-4-(trifluoromethyl)picolinamide (83 mg, 0.205 mmol), Pd(dppf)Cl₂ (14 mg, 0.0186 mmol), Cs₂CO₃ (181 mg, 0.558 mmol). The mixture was stirred for 2 h at 120° C. under N₂. The reaction mixture was diluted with EA (30 mL). The organic layer was washed with H₂O (10 mL×3), brine (10 mL×3), dried over Na₂SO₄, concentrated under vacuum. The residue was purified by column chromatography (EA=100%) to afford N-(3-(2-((4-fluorophenyl)amino)-8,9-dihydroimidazo[1′,2′:1,6]pyrido[2,3-d]pyrimidin-6-yl)-4-methylphenyl)-4-(trifluoromethyl)picolinamide (43.1 mg, 41% yield) as a yellow solid.

¹H NMR (400 MHz, DMSO-d₆): δ 10.77 (s, 1H), 9.89 (s, 1H), 9.03 (d, J=5.2 Hz, 1H), 8.39 (s, 1H), 8.33 (s, 1H), 8.09 (d, J=4.4 Hz, 1H), 7.86-7.82 (m, 3H), 7.77 (d, J=2.4 Hz, 1H), 7.25 (d, J=8.0 Hz, 2H), 7.15 (t, J=8.8 Hz, 2H), 4.17-4.13 (m, 2H), 3.99-3.95 (m, 2H), 2.22 (s, 3H). LCMS (M+H⁺) m/z: 560.5.

### Example 228: Preparation of N-(3-(2-((3-fluoro-4-methylphenyl)amino)-8,9-dihydroimidazo[1′,2′:1,6]pyrido[2,3-d]pyrimidin-6-yl)-4-methylphenyl)-4-(trifluoromethyl)picolinamide (Compound 228)

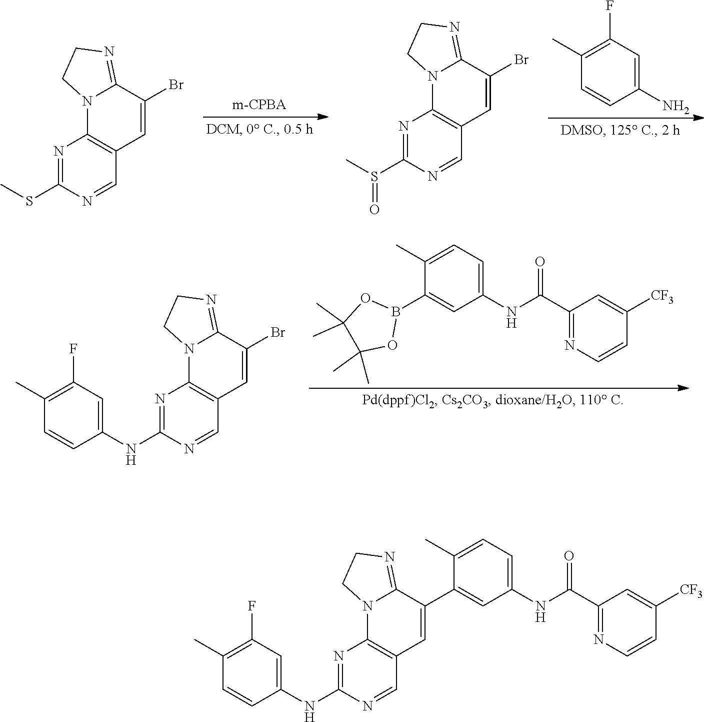

### Step 1: Synthesis of 6-bromo-N-(3-fluoro-4-methylphenyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (113 mg, 0.36 mmol) in DMSO (5 mL) was added 3-fluoroaniline (90 mg, 0.72 mmol). The reaction mixture was stirred for 2 h at 125° C. The reaction mixture was diluted with EA (30 mL), washed with water (10 mL×3), brine (10 mL×3), dried over Na₂SO₄, concentrated under vacuum. The residue was triturated with EA (10 mL) to obtain 6-bromo-N-(3-fluoro-4-methylphenyl)-8,9-dihydroimidazo[1′,2′:1,6]pyrido[2,3-d]pyrimidin-2-amine (67 mg, 50% yield) as a yellow solid. LCMS (M+H⁺) m/z: 374.0 and 376.0.

### Step 2: Synthesis of N-(3-(2-((3-fluoro-4-methylphenyl)amino)-8,9-dihydroimidazo[1′,2′:1,6]pyrido[2,3-d]pyrimidin-6-yl)-4-methylphenyl)-4-(trifluoromethyl)picolinamide

To a solution of 6-bromo-N-(3-fluorophenyl)-8,9-dihydroimidazo[1′,2′:1,6]pyrido[2,3-d]pyrimidin-2-amine (67 mg, 0.178 mmol) in dioxane/H₂O (7 mL/3 mL) was added N-(4-methyl-3-(4,4,5,5-tetramethyl-1,3,2-dioxaborolan-2-yl)phenyl)-4-(trifluoromethyl)picolinamide (79 mg, 0.196 mmol), Pd(dppf)Cl₂ (13 mg, 0.018 mmol), Cs₂CO₃ (173 mg, 0.534 mmol), the mixture was stirred for 2 h at 120° C. under N₂. The reaction mixture was diluted with EA (30 mL). The organic layer was washed with H₂O (10 mL×3), brine (10 mL×3), dried over Na₂SO₄, concentrated under vacuum. The residue was purified by column chromatography (EA=100%), followed by trituration with MeOH (3 mL) to afford N-(3-(2-((3-fluoro-4-methylphenyl)amino)-8,9-dihydroimidazo[1′,2′:1,6]pyrido[2,3-d]pyrimidin-6-yl)-4-methylphenyl)-4-(trifluoromethyl)picolinamide (15.2 mg, 15% yield) as a yellow solid.

¹H NMR (400 MHz, DMSO-d₆): δ 10.77 (s, 1H), 9.93 (s, 1H), 9.03 (d, J=5.2 Hz, 1H), 8.39 (s, 1H), 8.34 (s, 1H), 8.09 (dd, J=5.2, 1.2 Hz, 1H), 7.86-7.77 (m, 3H), 7.45 (dd, J=8.4, 2.0 Hz, 1H), 7.26-7.15 (m, 3H), 4.14 (t, J=9.6 Hz, 2H), 3.98 (t, J=9.6 Hz, 2H), 2.23 (s, 3H), 2.18 (s, 3H). LCMS (M+H⁺) m/z: 574.4.

### Example 229: Preparation of N-(3-(2-((3-(hydroxymethyl)phenyl)amino)-8,9-dihydroimidazo[1′,2′:1,6]pyrido[2,3-d]pyrimidin-6-yl)-4-methylphenyl)-4-(trifluoromethyl)picolinamide (Compound 229)

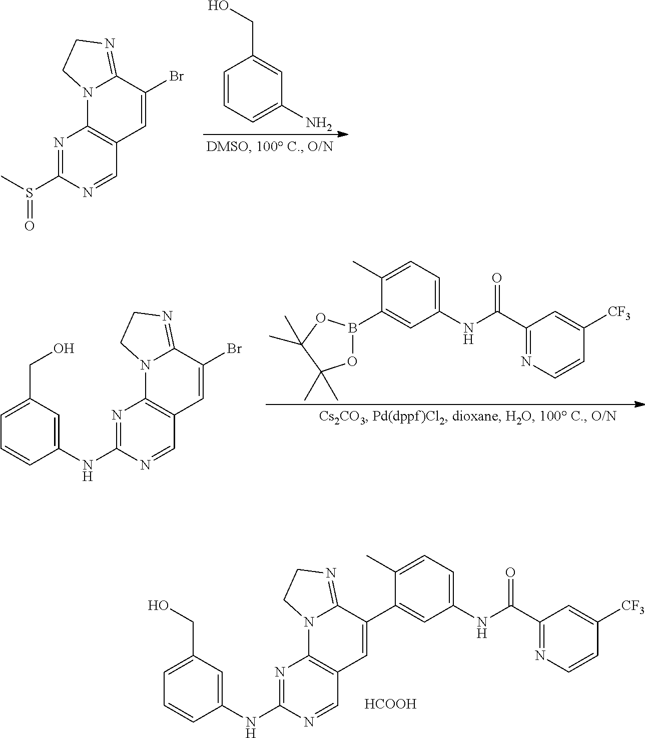

### Step 1: Synthesis of (3-((6-bromo-8,9-dihydroimidazo[1′,2′:1,6]pyrido[2,3-d]pyrimidin-2-yl)amino)phenyl) methanol

To a solution of 6-bromo-2-(methylsulfinyl)-8,9-dihydroimidazo[1′,2′:1,6]pyrido[2,3-d]pyrimidine (crude, 432 mg, 0.68 mmol) in DMSO (10 mL) was added (3-aminophenyl)methanol (125 mg, 1.02 mmol). The mixture was stirred at 120° C. for 16 h. The reaction was cooled to r.t. and diluted with water (50 mL), the reaction mixture was extracted with EA (50 mL×3). The combined organic phase was washed with brine (50 mL×3), dried with Na₂SO₄, filtered and concentrated. The residue was purified by trituration with EA (5.0 mL) to afford (3-((6-bromo-8,9-dihydroimidazo[1′,2′:1,6]pyrido[2,3-d]pyrimidin-2-yl)amino)phenyl) methanol (200 mg, 79% yield) as a yellow solid. LCMS (M+H⁺) m/z: 372.0 and 374.0.

### Step 2: Synthesis of N-(3-(2-((3-(hydroxymethyl)phenyl)amino)-8,9-dihydroimidazo[1′,2′:1,6]pyrido[2,3-d]pyrimidin-6-yl)-4-methylphenyl)-4-(trifluoromethyl)picolinamide formic acid

A mixture of (3-((6-bromo-8,9-dihydroimidazo[1′,2′:1,6]pyrido[2,3-d]pyrimidin-2-yl)amino)phenyl)methanol (150 mg, 0.40 mmol), N-(4-methyl-3-(4,4,5,5-tetramethyl-1,3,2-dioxaborolan-2-yl)phenyl)-4-(trifluoromethyl)picolinamide (195 mg, 0.48 mmol), Cs₂CO₃ (395 mg, 1.21 mmol) and Pd(dppf)Cl₂ (15 mg, 0.02 mmol) in dioxane (10 mL) and water (1 mL) was degassed and charged with N₂ three times, stirred at 100° C. for 16 h. The reaction mixture was concentrated and purified by Prep-HPLC (0.1% FA) to afford N-(3-(2-((3-(hydroxymethyl)phenyl)amino)-8,9-dihydroimidazo[1′,2′:1,6]pyrido[2,3-d]pyrimidin-6-yl)-4-methylphenyl)-4-(trifluoromethyl)picolinamide formic acid (29.7 mg, 13% yield) as a yellow solid.

¹H NMR (400 MHz, DMSO-d₆): δ 10.79 (s, 1H), 9.96 (br, 1H), 9.04 (d, J=5.2 Hz, 1H), 8.49 (s, 1H), 8.34 (s, 1H), 8.14 (s, 1H), 8.09 (d, J=4.8 Hz, 1H), 7.89 (s, 2H), 7.80 (dd, J=8.4, 2.4 Hz, 1H), 7.68 (d, J=8.8 Hz, 1H), 7.39-7.35 (m, 1H), 7.29-7.24 (m, 2H), 6.96 (d, J=7.6 Hz, 1H), 5.17-5.15 (m, 1H), 4.9 (d, J=3.2 Hz, 2H), 4.26-4.21 (m, 2H), 3.99 (t, J=9.6 Hz, 2H), 2.23 (s, 3H). LCMS (M+H⁺) m/z: 572.5.

### Example 230: Preparation of N-(4-methyl-3-(2-((pyridin-3-ylmethyl)amino)-8,9-dihydroimidazo[1′,2′:1,6]pyrido[2,3-d]pyrimidin-6-yl)phenyl)-4-(trifluoromethyl)picolinamidee (Compound 230)

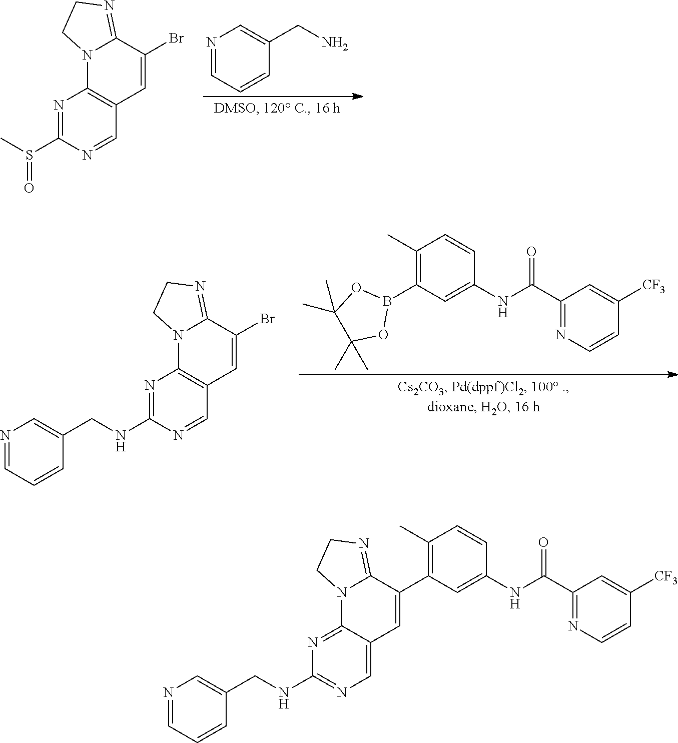

### Step 1: Synthesis of 6-bromo-N-(pyridin-3-ylmethyl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (crude, 50 mg, 0.16 mmol) in DMSO (5 mL) was added pyridin-3-ylmethanamine (35 mg, 0.32 mmol). The mixture was stirred at 120° C. for 16 h. The reaction was cooled to r.t. and purified by Prep-HPLC (0.1% NH₃—H₂O) to afford 6-bromo-N-(pyridin-3-ylmethyl)-8,9-dihydroimidazo[1′,2′:1,6]pyrido[2,3-d]pyrimidin-2-amine (40 mg, 70% yield) as brown solid. LCMS (M+H⁺) m/z: 357.0 and 359.0.

### Step 2: Synthesis of N-(4-methyl-3-(2-((pyridin-3-ylmethyl)amino)-8,9-dihydroimidazo[1′,2′:1,6]pyrido[2,3-d]pyrimidin-6-yl)phenyl)-4-(trifluoromethyl)picolinamide

A mixture of 6-bromo-N-(pyridin-3-ylmethyl)-8,9-dihydroimidazo[1′,2′:1,6]pyrido[2,3-d]pyrimidin-2-amine (40 mg, 0.108 mmol), N-(4-methyl-3-(4,4,5,5-tetramethyl-1,3,2-dioxaborolan-2-yl)phenyl)-4-(trifluoromethyl)picolinamide (48 mg, 0.118 mmol), Cs₂CO₃ (105 mg, 0.323 mmol) and Pd(dppf)Cl₂ (4 mg, 0.005 mmol) in dioxane (5 mL) and water (0.5 mL) was degassed and charged with N₂ three times and stirred at 100° C. for 16 h. The reaction mixture was concentrated in vacuum and purified by column chromatography on silica gel (DCM/MeOH=10/1) to give crude product, which was purified by Prep-HPLC (0.1% NH₃—H₂O) to afford N-(4-methyl-3-(2-((pyridin-3-ylmethyl)amino)-8,9-dihydroimidazo[1′,2′:1,6]pyrido[2,3-d]pyrimidin-6-yl)phenyl)-4-(trifluoromethyl)picolinamide (10.0 mg, 22.8% yield) as a yellow solid.

¹H NMR (400 MHz, DMSO-d₆): δ 10.74 (s, 1H), 9.03 (d, J=5.2 Hz, 1H), 8.61-8.59 (m, 1H), 8.44 (d, J=4.0 Hz, 1H), 8.33 (s, 1H), 8.21 (s, 1H), 8.13-8.08 (m, 2H), 7.79-7.71 (m, 3H), 7.35 (s, 1H), 7.22 (d, J=8.4 Hz, 1H), 7.12 (s, 1H), 4.53 (s, 2H), 4.01 (t, J=8.4 Hz, 2H), 3.90 (t, J=8.4 Hz, 2H), 2.19 (s, 3H). LCMS (M+H⁺) m/z: 557.3.

### Example 231: Preparation of N-(4-methyl-3-(2-((2-methylthiazol-5-yl)amino)-8,9-dihydroimidazo[1′,2′:1,6]pyrido[2,3-d]pyrimidin-6-yl)phenyl)-4-(trifluoromethyl)picolinamide (Compound 231)

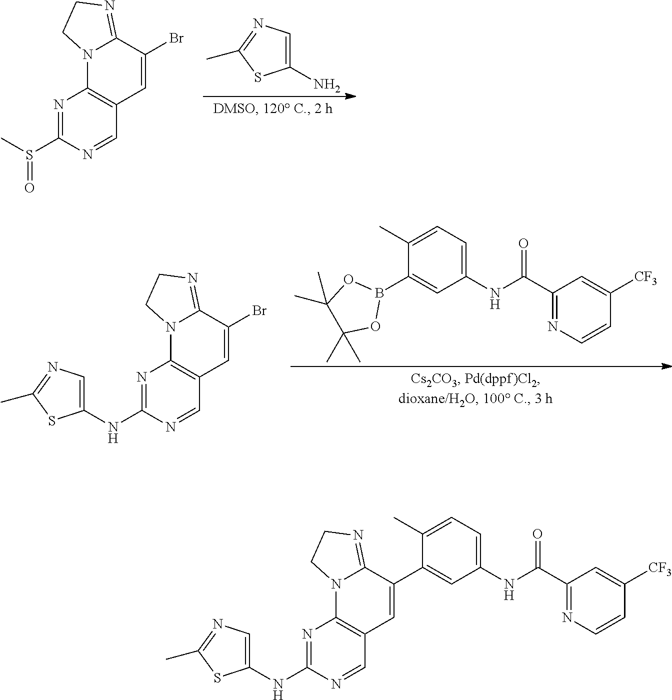

### Step 1: Synthesis of N-(6-bromo-8,9-dihydroimidazo[1′,2′:1,6]pyrido[2,3-d]pyrimidin-2-yl)-2-methylthiazol-5-amine

To a solution of 6-bromo-2-(methylsulfinyl)-8,9-dihydroimidazo[1′,2′:1,6]pyrido[2,3-d]pyrimidine (500 mg, 1.60 mmol) in DMSO (30 mL) was added 2-methylthiazol-5-amine (273 mg, 2.40 mmol), the mixture was stirred for 2 h at 120° C. The reaction mixture was removed in vacuum. The residue was purified by column chromatography (DCM:MeOH=20:1) to afford N-(6-bromo-8,9-dihydroimidazo[1′,2′:1,6]pyrido[2,3-d]pyrimidin-2-yl)-2-methylthiazol-5-amine (250 mg, 39.2% yield) as a brown solid. LCMS (M+H⁺) m/z: 363.1 and 365.1.

### Step 2: Synthesis of N-(4-methyl-3-(2-((2-methylthiazol-5-yl)amino)-8,9-dihydroimidazo[1′,2′:1,6]pyrido[2,3-d]pyrimidin-6-yl)phenyl)-4-(trifluoromethyl)picolinamide

To a solution of N-(6-bromo-8,9-dihydroimidazo[1′,2′:1,6]pyrido[2,3-d]pyrimidin-2-yl)-2-methylthiazol-5-amine (200 mg, 0.55 mmol) in dioxane/H₂O (120 mL/20 mL) was added N-(4-methyl-3-(4,4,5,5-tetramethyl-1,3,2-dioxaborolan-2-yl)phenyl)-4-(trifluoromethyl)picolinamide (224 mg, 0.55 mmol), Pd(dppf)Cl₂ (40 mg, 0.055 mmol), Cs₂CO₃ (539 mg, 1.65 mmol), the mixture was stirred for 3 h at 100° C. under N₂. The reaction mixture was concentrated and purified by Prep-HPLC (0.1% NH₃H₂O) to afford N-(4-methyl-3-(2-((2-methylthiazol-5-yl)amino)-8,9-dihydroimidazo[1′,2′:1,6]pyrido[2,3-d]pyrimidin-6-yl)phenyl)-4-(trifluoromethyl)picolinamide (63.2 mg, 20.4% yield) as a yellow solid.

¹H NMR (400 MHz, DMSO-d₆): δ 12.07 (br, 1H), 10.94 (s, 1H), 10.12 (s, 1H), 9.11 (s, 1H), 9.05 (d, J=4.8 Hz, 1H), 8.34 (s, 1H), 8.27 (s, 1H), 8.12 (d, J=4.4 Hz, 1H), 8.04 (s, 1H), 7.92 (d, J=8.4 Hz, 1H), 7.60 (s, 1H), 7.43 (d, J=8.4 Hz, 1H), 4.82 (t, J=9.2 Hz, 2H), 4.14 (t, J=9.2 Hz, 2H), 2.67 (s, 3H), 2.23 (s, 3H). LCMS (M+H⁺) m/z: 563.3.

### Example 232: Preparation of N-(4-methyl-3-(2-((5-methylthiazol-2-yl)amino)-8,9-dihydroimidazo[1′,2′:1,6]pyrido[2,3-d]pyrimidin-6-yl)phenyl)-4-(trifluoromethyl)picolinamide (Compound 232)

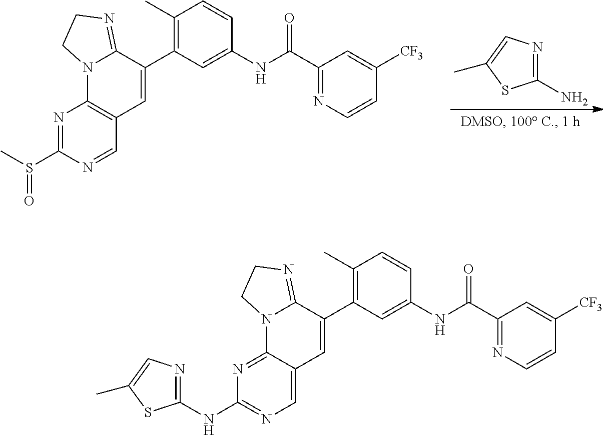

The preparation of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide was described in Example 106

### Step 1: Synthesis of N-(4-methyl-3-(2-((5-methylthiazol-2-yl)amino)-8,9-dihydroimidazo[1′,2′:1,6]pyrido[2,3-d]pyrimidin-6-yl)phenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (150 mg, crude), DIEA (0.3 mL) and 5-methylthiazol-2-amine (100 mg) in DMSO (1 mL) was stirred at 100° C. for 1 h. The mixture was purified by Prep-HPLC (0.1% FA) to afford N-(4-methyl-3-(2-((5-methylthiazol-2-yl)amino)-8,9-dihydroimidazo[1′,2′:1,6]pyrido[2,3-d]pyrimidin-6-yl)phenyl)-4-(trifluoromethyl)picolinamide (6.4 mg) as a yellow solid. ¹H NMR (400 MHz, DMSO-d₆): δ 11.64 (s, 1H), 10.79 (s, 1H), 9.03 (d, J=5.2 Hz, 1H), 8.45 (s, 1H), 8.34 (s, 1H), 8.10 (dd, J=5.2, 1.2 Hz, 1H), 7.87 (d, J=2.0 Hz, 1H), 7.79 (dd, J=8.4, 2.0 Hz, 1H), 7.28-7.25 (m, 2H), 7.12 (d, J=1.2 Hz, 1H), 4.26-4.21 (m, 2H), 4.04-3.99 (m, 2H), 2.36 (s, 3H), 2.23 (s, 3H). LCMS (M+H⁺) m/z: 562.9.

### Example 233: Preparation of N-(4-methyl-3-(2-((4-methylthiazol-2-yl)amino)-8,9-dihydroimidazo[1′,2′:1,6]pyrido[2,3-d]pyrimidin-6-yl)phenyl)-4-(trifluoromethyl)picolinamide (Compound 233)

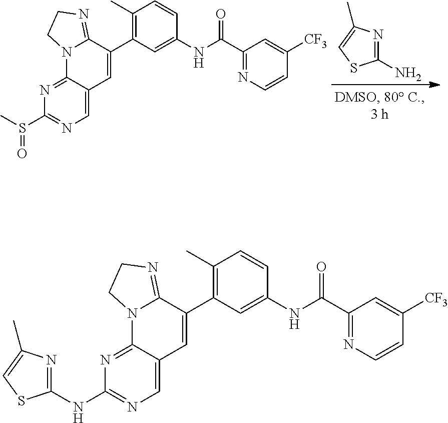

### Step 1: Synthesis of N-(4-methyl-3-(2-((4-methylthiazol-2-yl)amino)-8,9-dihydroimidazo[1′,2′:1,6]pyrido[2,3-d]pyrimidin-6-yl)phenyl)-4-(trifluoromethyl)picolinamide

To a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (100 mg, crude) in DMSO (8 drops) was added 4-methylthiazol-2-amine (342 mg, 3 mmol). The mixture was stirred at 80° C. for 3 h. LCMS showed the reaction completed. The mixture was diluted to water (10 mL), extracted by EA (10 mL×3). The combined organic phase was washed by brine (20 mL), dried over anhydrous Na₂SO₄, filtered and concentrated to afford a crude solid, which was purified by prep-HPLC to afford N-(4-methyl-3-(2-((4-methylthiazol-2-yl)amino)-8,9-dihydroimidazo[1′,2′:1,6]pyrido[2,3-d]pyrimidin-6-yl)phenyl)-4-(trifluoromethyl)picolinamide (3.2 mg, 4% yield) as yellow solid.

1H NMR (400 MHz, DMSO-d6): δ 11.76 (br s, 1H), 10.78 (s, 1H), 9.03 (d, J=4.8 Hz, 1H), 8.47 (s, 1H), 8.33 (s, 1H), 8.09 (d, J=3.6 Hz, 1H), 7.87 (d, J=2.0 Hz, 1H), 7.79 (dd, J=8.4, 2.0 Hz, 1H), 7.31 (br, 1H), 7.26 (d, J=8.4 Hz, 2H), 6.73 (s, 1H), 4.26-4.23 (m, 2H), 4.00 (t, J=9.2 Hz, 2H), 2.27 (s, 3H), 2.23 (s, 3H).

### Example 234: Preparation of N-(4-methyl-3-(2-((1-methyl-1H-pyrazol-3-yl)amino)-8,9-dihydroimidazo[1′,2′:1,6]pyrido[2,3-d]pyrimidin-6-yl)phenyl)-4-(trifluoromethyl)picolinamide (Compound 234)

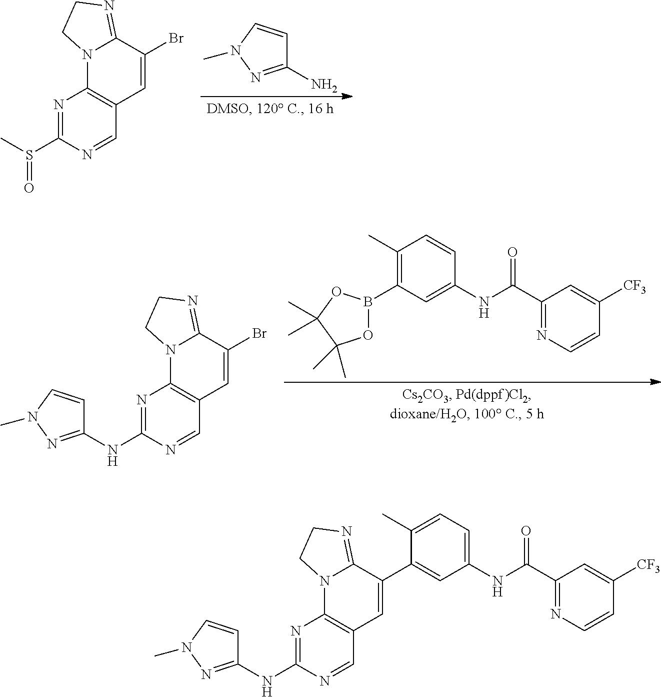

### Step 1: Synthesis of 6-bromo-N-(1-methyl-1H-pyrazol-3-yl)-8,9-dihydroimidazo[1′,2′:1,6]pyrido[2,3-d]pyrimidin-2-amine

To a solution of 6-bromo-2-(methylsulfinyl)-8,9-dihydroimidazo[1′,2′:1,6]pyrido[2,3-d]pyrimidine (5.5 g, 17.5 mmol) in DMSO (20 mL) was added 1-methyl-1H-pyrazol-3-amine (3.4 g, 35 mmol). The resulting mixture was stirred at 120° C. for 16 h. The reaction mixture was cooled and filtered to afford 6-bromo-N-(1-methyl-1H-pyrazol-3-yl)-8,9-dihydroimidazo[1′,2′:1,6]pyrido[2,3-d]pyrimidin-2-amine (4 g, 67% yield) as a yellow solid. LCMS (M+H⁺) m/z: 346.1 and 381.1.

### Step 2: Synthesis of N-(4-methyl-3-(2-((1-methyl-1H-pyrazol-3-yl)amino)-8,9-dihydroimidazo[1′,2′:1,6]pyrido[2,3-d]pyrimidin-6-yl)phenyl)-4-(trifluoromethyl)picolinamide hydrochloride

To a mixture of 6-bromo-N-(1-methyl-1H-pyrazol-3-yl)-8,9-dihydroimidazo[1′,2′:1,6]pyrido[2,3-d]pyrimidin-2-amine (4 g, 11.6 mmol) in dioxane/H₂O (10:1) (250 mL) was added N-(4-methyl-3-(4,4,5,5-tetramethyl-1,3,2-dioxaborolan-2-yl)phenyl)-4-(trifluoromethyl)picolinamide (4.7 g, 11.6 mmol), Pd(dppf)Cl₂ (850 mg, 1.16 mmol), Cs₂CO₃ (11.3 g, 34.8 mmol). The mixture was degassed three times and charged with N₂, stirred at 110° C. for 5 hrs. The reaction mixture was concentrated in vacuum and purified by column chromatography (DCM/MeOH=10/1) to afford the crude product, then trituration with EA (200 mL) twice gave N-(4-methyl-3-(2-((1-methyl-1H-pyrazol-3-yl)amino)-8,9-dihydroimidazo[1′,2′:1,6]pyrido[2,3-d]pyrimidin-6-yl)phenyl)-4-(trifluoromethyl)picolinamide hydrochloride (4494 mg, 71% yield) as a yellow solid.

¹H NMR (400 MHz, DMSO-d₆): δ 11.02 (s, 1H), 10.93 (s, 1H), 9.97 (s, 1H), 9.06-9.05 (m, 2H), 8.34 (s, 1H), 8.23 (s, 1H), 8.12-8.11 (m, 1H), 8.02 (d, J=2.4 Hz, 1H), 7.92 (dd, J=8.4, 2.0 Hz, 1H), 7.71 (s, 1H), 7.42 (d, J=8.4 Hz, 1H), 6.81 (s, 1H), 4.73 (t, J=9.6 Hz, 2H), 4.07 (t, J=10.0 Hz, 2H), 3.81 (s, 3H), 2.22 (s, 3H). LCMS (M+H⁺) m/z: 546.3.

### Example 235: Preparation of N-(4-methyl-3-(2-((1-methyl-1H-pyrazol-5-yl)amino)-8,9-dihydroimidazo[1′,2′:1,6]pyrido[2,3-d]pyrimidin-6-yl)phenyl)-4-(trifluoromethyl)picolinamide (Compound 235)

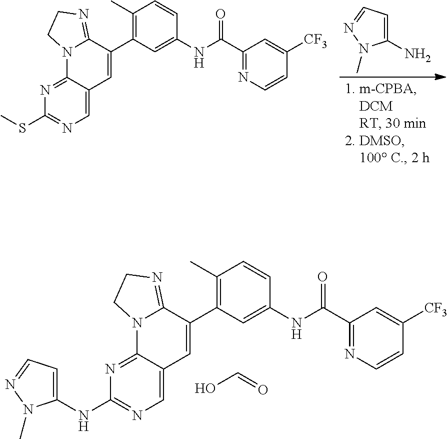

### Step 1: Synthesis of N-(4-methyl-3-(2-((1-methyl-1H-pyrazol-5-yl)amino)-8,9-dihydroimidazo[1′,2′:1,6]pyrido[2,3-d]pyrimidin-6-yl)phenyl)-4-(trifluoromethyl)picolinamide

To a mixture of N-(4-methyl-3-(2-(methylthio)-8,9-dihydroimidazo[1′,2′:1,6]pyrido[2,3-d]pyrimidin-6-yl)phenyl)-4-(trifluoromethyl)picolinamide (100 mg, 0.2 mmol) in DCM (10 mL), was added m-CPBA (86 mg, 0.5 mmol). The mixture was stirred for 0.5 h at 25° C. The reaction was concentrated and a solution of 1-methyl-1H-pyrazol-5-amine (97 mg, 1.0 mmol) in DMSO (0.5 mL) was added. The reaction mixture was stirred at 100° C. for 5 h and purified by flash (DCM:MeOH=10:1) and Prep-HPLC (0.5% FA) to give N-(4-methyl-3-(2-((1-methyl-1H-pyrazol-5-yl)amino)-8,9-dihydroimidazo[1′,2′:1,6]pyrido[2,3-d]pyrimidin-6-yl)phenyl)-4-(trifluoromethyl)picolinamide (6.8 mg, 6% yield) as yellow solid.

¹H NMR (400 MHz, CD₃OD): δ 8.99 (d, J=4.8 Hz, 1H), 8.82 (d, J=4.0 Hz, 1H), 8.67 (s, 1H), 8.44 (s, 1H), 8.37 (s, 1H), 7.95 (d, J=4.0 Hz, 1H), 7.90 (d, J=2.0 Hz, 1H), 7.75 (dd, J=8.4, 2.0 Hz, 1H), 7.52 (s, 1H), 7.37 (d, J=8.0 Hz, 1H), 6.28 (d, J=4.0 Hz, 1H), 4.40 (t, J=9.6 Hz, 2H), 4.17-4.12 (m, 5H), 2.32 (s, 3H). LCMS (M+H⁺) m/z: 546.0.

### Example 236: Preparation of N-(3-(2-((1,3-dimethyl-1H-pyrazol-5-yl)amino)-8,9-dihydroimidazo[1′,2′:1,6]pyrido[2,3-d]pyrimidin-6-yl)-4-methylphenyl)-4-(trifluoromethyl)picolinamide (Compound 236)

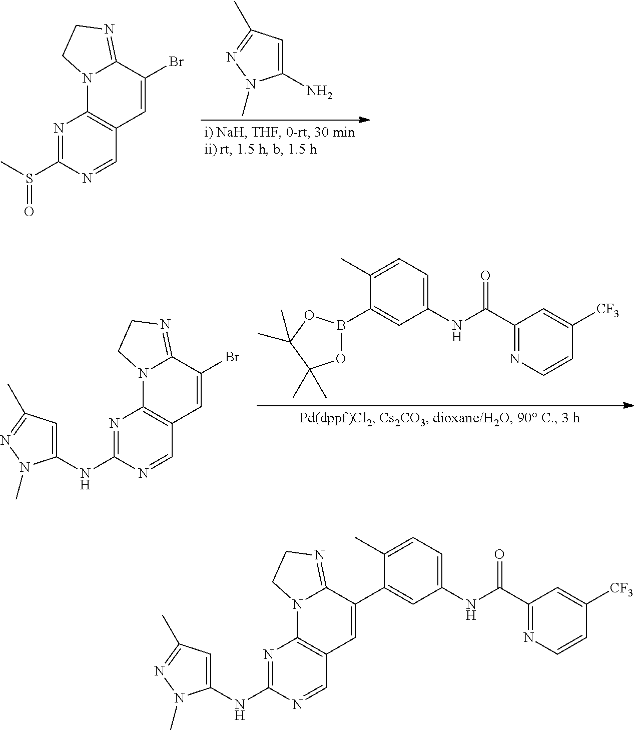

### Step 1: Synthesis of 6-bromo-N-(1,3-dimethyl-1H-pyrazol-5-yl)-8,9-dihydroimidazo[1′,2′:1,6]pyrido[2,3-d]pyrimidin-2-amine

To a mixture of 1,3-dimethyl-1H-pyrazol-5-amine (112 mg, 1.01 mmol) in dry DMF (6.0 mL) was added NaH (54 mg, 1.34 mmol) at 0° C. The resulting mixture was stirred at r.t for 0.5 h and 6-bromo-2-(methylsulfinyl)-8,9-dihydroimidazo[1′,2′:1,6]pyrido[2,3-d]pyrimidine (210 mg, 0.67 mmol) was added, the mixture was stirred at r.t for 1.5 h. The reaction mixture was quenched with water, extracted with DCM (50 mL×3). The combined organic phase was washed with brine, dried over Na2SO4, filtered. The residue was purified by column chromatography (DCM/MeOH=10/1) to afford crude product, which was triturated with EA/PE (1/1, 2.0 mL) to give 6-bromo-N-(1,3-dimethyl-1H-pyrazol-5-yl)-8,9-dihydroimidazo[1′,2′:1,6]pyrido[2,3-d]pyrimidin-2-amine (12 mg, 5% yield) as a yellow solid. LCMS (M+H⁺) m/z: 358.0 and 360.0.

### Step 2: Synthesis of N-(3-(2-((1,3-dimethyl-1H-pyrazol-5-yl)amino)-8,9-dihydroimidazo[1′,2′:1,6]pyrido[2,3-d]pyrimidin-6-yl)-4-methylphenyl)-4-(trifluoromethyl)picolinamide hydrogen chloride

A mixture of 6-bromo-N-(1,3-dimethyl-1H-pyrazol-5-yl)-8,9-dihydroimidazo[1′,2′:1,6]pyrido[2,3-d]pyrimidin-2-amine (10 mg, 0.03 mmol), N-(4-methyl-3-(4,4,5,5-tetramethyl-1,3,2-dioxaborolan-2-yl)phenyl)-4-(trifluoromethyl)picolinamide (13.5 mg, 0.033 mmol), Cs2CO3 (19.6 mg, 0.06 mmol) and Pd(dppf)Cl₂ (2.3 mg, 0.003 mmol) in dioxane (1.0 mL) and H₂O (0.1 mL) was degassed, charged with N₂ for 3 times and stirred at 90° C. for 3 h. The reaction mixture was concentrated and purified by Prep-TLC (DCM/MeOH=10/1) and Prep-HPLC (0.1% HCl) to afford N-(3-(2-((1,3-dimethyl-1H-pyrazol-5-yl)amino)-8,9-dihydroimidazo[1′,2′:1,6]pyrido[2,3-d]pyrimidin-6-yl)-4-methylphenyl)-4-(trifluoromethyl)picolinamide hydrogen chloride (0.96 mg, 6.1% yield) as a yellow solid.

¹H NMR (400 MHz, CD₃OD): δ 9.04 (s, 1H), 8.97 (d, J=4.8 Hz, 1H), 8.42 (s, 1H), 8.20 (s, 1H), 7.95-7.93 (m, 2H), 7.79 (dd, J=8.4, 2.4 Hz, 1H), 7.46 (d, J=8.4 Hz, 1H), 6.43 (s, 1H), 4.77-4.72 (m, 2H), 4.17 (t, J=10.0 Hz, 2H), 3.79 (s, 3H), 2.30 (s, 3H), 2.29 (s, 3H). LCMS (M+H⁺) m/z: 560.3.

### Example 237: Preparation of N-(4-methyl-3-(2-((1-methyl-1H-1,2,3-triazol-4-yl)amino)-8,9-dihydroimidazo[1′,2′:1,6]pyrido[2,3-d]pyrimidin-6-yl)phenyl)-4-(trifluoromethyl)picolinamide (Compound 237)

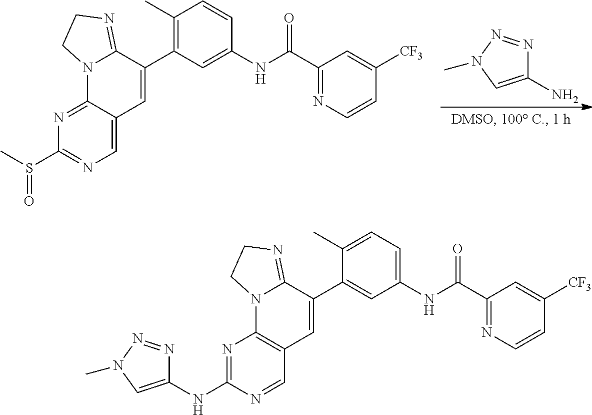

### Step 1: Synthesis of N-(4-methyl-3-(2-((1-methyl-1H-1,2,3-triazol-4-yl)amino)-8,9-dihydroimidazo[1′,2′:1,6]pyrido[2,3-d]pyrimidin-6-yl)phenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (51 mg, 0.1 mmol), 1-methyl-1H-1,2,3-triazol-4-amine (98 mg, 1.0 mmol) in DMSO (0.2 mL) was stirred at 100° C. for 1 h. The reaction was purified by flash (DCM:MeOH=10:1) and Prep-HPLC (0.5% F A) to give N-(4-methyl-3-(2-((1-methyl-1H-1,2,3-triazol-4-yl)amino)-8,9-dihydroimidazo[1′,2′:1,6]pyrido[2,3-d]pyrimidin-6-yl)phenyl)-4-(trifluoromethyl)picolinamide (5.4 mg, 10% yield) as yellow solid.

¹H NMR (400 MHz, DMSO-d₆): δ 10.79 (s, 1H), 10.56 (s, 1H), 9.04 (d, J=5.2 Hz, 1H), 8.38 (s, 1H), 8.33 (s, 1H), 8.17-8.16 (m, 1H), 8.09 (dd, J=5.2, 1.2 Hz, 1H), 7.86 (d, J=2.4 Hz, 1H), 7.78 (dd, J=8.4, 2.0 Hz, 1H), 7.26-7.24 (m, 2H), 4.20-4.19 (m, 2H), 4.07 (s, 3H), 4.00-3.96 (m, 2H), 2.22 (s, 3H). LCMS (M+H⁺) m/z: 547.0.

### Example 238: Preparation of N-(4-methyl-3-(2-((1-methyl-1H-1,2,4-triazol-3-yl)amino)-8,9-dihydroimidazo[1′,2′:1,6]pyrido[2,3-d]pyrimidin-6-yl)phenyl)-4-(trifluoromethyl)picolinamide (compound 238)

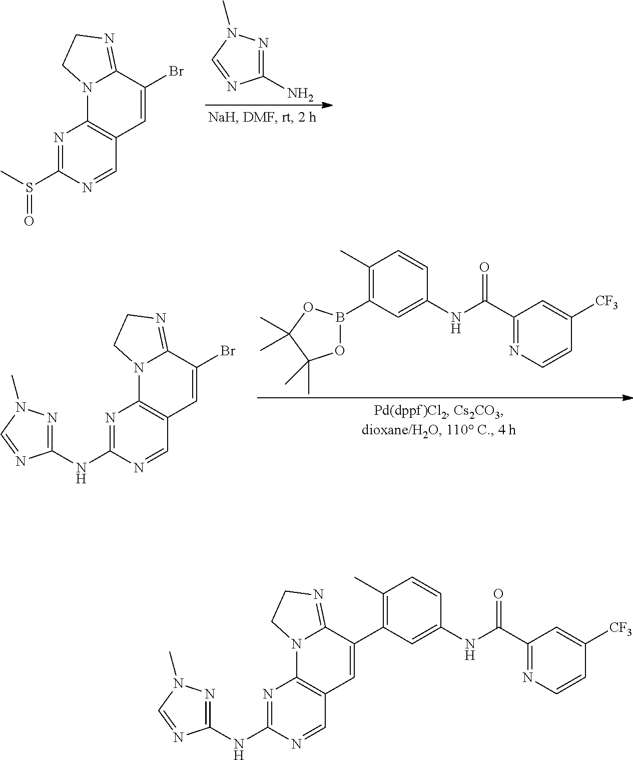

### Step 1: Synthesis of 6-bromo-N-(1-methyl-1H-1,2,4-triazol-3-yl)-8,9-dihydroimidazo[1′,2′:1,6]pyrido[2,3-d]pyrimidin-2-amine

To a solution of 1-methyl-1H-1,2,4-triazol-3-amine (150 mg, 1.53 mmol) in DMF (6 mL) was added NaH (123 mg, 3.07 mmol, 60% wt in mineral oil). The reaction mixture was stirred at R.T under N₂ for 1 hour. Then a solution of 6-bromo-2-(methylsulfinyl)-8,9-dihydroimidazo[1′,2′:1,6]pyrido[2,3-d]pyrimidine (300 mg, 0.96 mmol) in DMF (18 mL) was added dropwise to above reaction solution. The result solution was stirred at R.T under N₂ for 1 hour, then quenched with sat. NH₄Cl and adjusted pH to 6.0 by 1M HCl. The solution was concentrated and the residue was triturated with DCM/MeOH=5/1 (10 mL). The filtrate was concentrated and purified by flash chromatography to afford 6-bromo-N-(1-methyl-1H-1,2,4-triazol-3-yl)-8,9-dihydroimidazo[1′,2′:1,6]pyrido[2,3-d]pyrimidin-2-amine (20 mg, 6% yield) as a yellow solid. LCMS (M+H⁺) m/z: 349.0.

### Step 2: Synthesis of N-(4-methyl-3-(2-((1-methyl-1H-1,2,4-triazol-3-yl)amino)-8,9-dihydroimidazo[1′,2′:1,6]pyrido[2,3-d]pyrimidin-6-yl)phenyl)-4-(trifluoromethyl)picolinamide formate

A mixture of 6-bromo-N-(1-methyl-1H-1,2,4-triazol-3-yl)-8,9-dihydroimidazo[1′,2′:1,6]pyrido[2,3-d]pyrimidin-2-amine (20 mg, 0.058 mmol), N-(4-methyl-3-(4,4,5,5-tetramethyl-1,3,2-dioxaborolan-2-yl)phenyl)-4-(trifluoromethyl)picolinamide (47 mg, 0.116 mmol), Cs₂CO₃ (57 mg, 0.174 mmol) and Pd(dppf)Cl₂ (13 mg, 0.0174 mmol) in dioxane (4 mL) and water (1 mL) was stirred at 105° C. under N₂ for 2 h. The reaction mixture was cooled to r.t. and directly purified by flash chromatography to get crude product. The crude product was purified by Prep-HPLC (0.1%/FA/CH₃CN/H₂O) to afford N-(4-methyl-3-(2-((1-methyl-1H-1,2,4-triazol-3-yl)amino)-8,9-dihydroimidazo[1′,2′:1,6]pyrido[2,3-d]pyrimidin-6-yl)phenyl)-4-(trifluoromethyl)picolinamide formate (2.54 mg, 7% yield) as a yellow solid.

¹H NMR (400 MHz, DMSO-d₆) δ 10.75 (s, 1H), 9.73 (s, 1H), 9.03 (d, J=4.4 Hz, 1H), 8.33-8.28 (m, 4H), 8.08 (d, J=4.0 Hz, 1H), 7.83 (s, 1H), 7.78 (d, J=7.6 Hz, 1H), 7.24 (d, J=8.8 Hz, 1H), 7.19 (s, 1H), 4.03-3.96 (m, 2H), 3.94-3.90 (m, 2H), 3.82 (s, 3H), 2.22 (s, 3H). LCMS (M+H⁺) m/z: 547.1

### Example 239: Preparation of N-(3-(2-((1H-tetrazol-5-yl)amino)-8,9-dihydroimidazo[1′,2′:1,6]pyrido[2,3-d]pyrimidin-6-yl)-4-methylphenyl)-4-(trifluoromethyl)picolinamide (Compound 239)

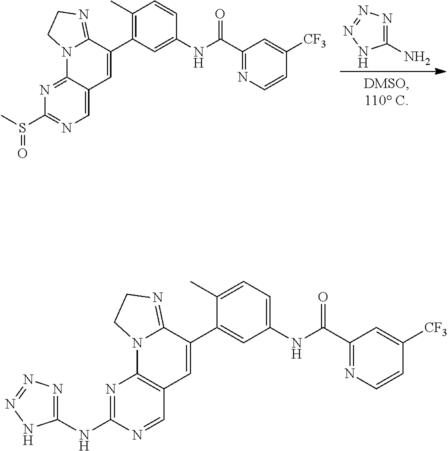 [SMILES: Cc1ccc(NC(=O)c2cc(C(F)(F)F)ccn2)cc1C1=Cc2cnc(S(C)=O)nc2N2CCN=C12 ; Nc1nnn[nH]1 ; Cc1ccc(NC(=O)c2cc(C(F)(F)F)ccn2)cc1C1=Cc2cnc(Nc3nnn[nH]3)nc2N2CCN=C12]

### Step 1: Synthesis of N-(3-(2-((1H-tetrazol-5-yl)amino)-8,9-dihydroimidazo[1′,2′:1,6]pyrido[2,3-d]pyrimidin-6-yl)-4-methylphenyl)-4-(trifluoromethyl)picolinamide

To a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (70 mg, crude) in DMSO (8 drops) was added 1H-tetrazol-5-amine (170 mg, 2 mmol). The mixture was stirred at 110° C. for 1 h. LCMS showed the reaction completed. The mixture was diluted with water (10 mL), extracted by EA (10 mL×3). The combined organic phase was washed by brine (20 mL), dried over anhydrous Na₂SO₄, filtered and concentrated to afford a crude solid, which was purified by prep-HPLC to afford N-(3-(2-((1H-tetrazol-5-yl)amino)-8,9-dihydroimidazo[1′,2′:1,6]pyrido[2,3-d]pyrimidin-6-yl)-4-methylphenyl)-4-(trifluoromethyl)picolinamide (10.2 mg) as yellow solid.

¹H NMR (400 MHz, DMSO-d₆): δ 11.55 (br, 1H), 10.83 (s, 1H), 9.04 (d, J=4.8 Hz, 1H), 8.55 (s, 1H), 8.34 (s, 1H), 8.14 (s, 1H), 8.10 (d, J=5.2 Hz, 1H), 7.90 (d, J=2.0 Hz, 1H), 7.81 (dd, J=8.4, 2.0 Hz, 1H), 7.49 (s, 1H), 7.29 (d, J=8.4 Hz, 1H), 4.23 (d, J=9.6 Hz, 2H), 3.98 (t, J=9.6 Hz, 2H), 2.22 (s, 3H). LCMS (M+H⁺) m/z: 533.9.

### Example 240 and 241: Preparation of N-(3-(2-((1H-pyrazol-5-yl)amino)-8,9-dihydroimidazo[1′,2′:1,6]pyrido[2,3-d]pyrimidin-6-yl)-4-methylphenyl)-4-(trifluoromethyl)picolinamide (Compound 240) and N-(3-(2-(5-amino-1H-pyrazol-1-yl)-8,9-dihydroimidazo[1′,2′:1,6]pyrido[2,3-d]pyrimidin-6-yl)-4-methylphenyl)-4-(trifluoromethyl)picolinamide (Compound 241)

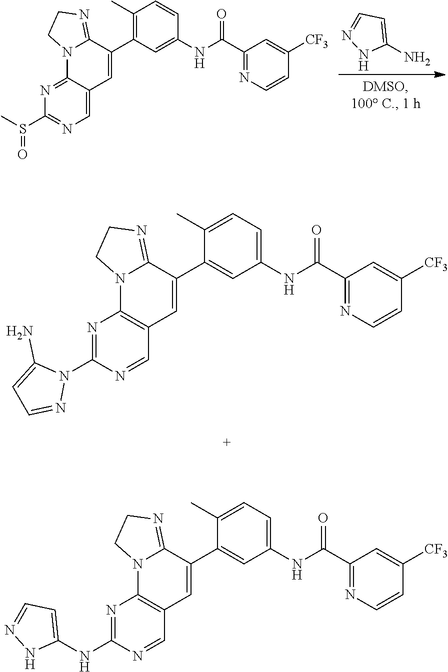

### Step 1: Synthesis of N-(3-(2-((1H-pyrazol-5-yl)amino)-8,9-dihydroimidazo[1′,2′:1,6]pyrido[2,3-d]pyrimidin-6-yl)-4-methylphenyl)-4-(trifluoromethyl)picolinamide and N-(3-(2-(5-amino-1H-pyrazol-1-yl)-8,9-dihydroimidazo[1′,2′:1,6]pyrido[2,3-d]pyrimidin-6-yl)-4-methylphenyl)-4-(trifluoromethyl)picolinamide

The mixture of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (128 mg, 0.4 mmol, crude), 1H-pyrazol-5-amine (332 mg, 4.0 mmol) was in DMSO (0.2 mL) was stirred at 100° C. for 2 h, The reaction was purified by flash (DCM:MeOH=10:1) to give N-(3-(2-((1H-pyrazol-5-yl)amino)-8,9-dihydroimidazo[1′,2′:1,6]pyrido[2,3-d]pyrimidin-6-yl)-4-methylphenyl)-4-(trifluoromethyl)picolinamide (12.5 mg), and N-(3-(2-(5-amino-1H-pyrazol-1-yl)-8,9-dihydroimidazo[1′,2′:1,6]pyrido[2,3-d]pyrimidin-6-yl)-4-methylphenyl)-4-(trifluoromethyl)picolinamide (7.3 mg)

Compound 241: ¹H NMR (400 MHz, DMSO-d₆): δ 10.80 (s, 1H), 9.04 (d, J=5.2 Hz, 1H), 8.51 (s, 1H), 8.35-8.33 (m, 2H), 8.10-8.08 (m, 1H), 7.89 (d, J=2.4 Hz, 1H), 7.79 (dd, J=8.0, 2.0 Hz, 1H), 7.34 (s, 1H), 7.27 (d, J=8.0 Hz, 1H), 5.89 (s, 1H), 5.51 (s, 2H), 4.15 (t, J=9.2 Hz, 2H), 3.98 (t, J=9.2 Hz, 2H), 2.23 (s, 3H). LCMS (M+H⁺) m/z: 531.9.

Compound 240: ¹H NMR (400 MHz, DMSO-d₆): δ 10.78 (s, 1H), 10.07 (s, 1H), 9.04 (d, J=4.8 Hz, 1H), 8.37 (s, 1H), 8.34 (s, 1H), 8.16 (s, 1H), 8.09 (dd, J=4.8, 1.2 Hz, 1H), 7.85 (d, J=2.4 Hz, 1H), 7.79 (dd, J=8.0, 2.0 Hz, 1H), 7.60 (d, J=2.0 Hz, 1H), 7.26-7.24 (m, 2H), 6.68 (br s, 1H), 4.16 (t, J=9.6 Hz, 2H), 3.95 (t, J=9.6 Hz, 2H), 2.22 (s, 3H). LCMS (M+H⁺) m/z: 532.0.

### Example 242: Preparation of N-(4-methyl-3-(2-((3-methylisothiazol-5-yl)amino)-8,9-dihydroimidazo[1′,2′:1,6]pyrido[2,3-d]pyrimidin-6-yl)phenyl)-4-(trifluoromethyl)picolinamide (Compound 242)

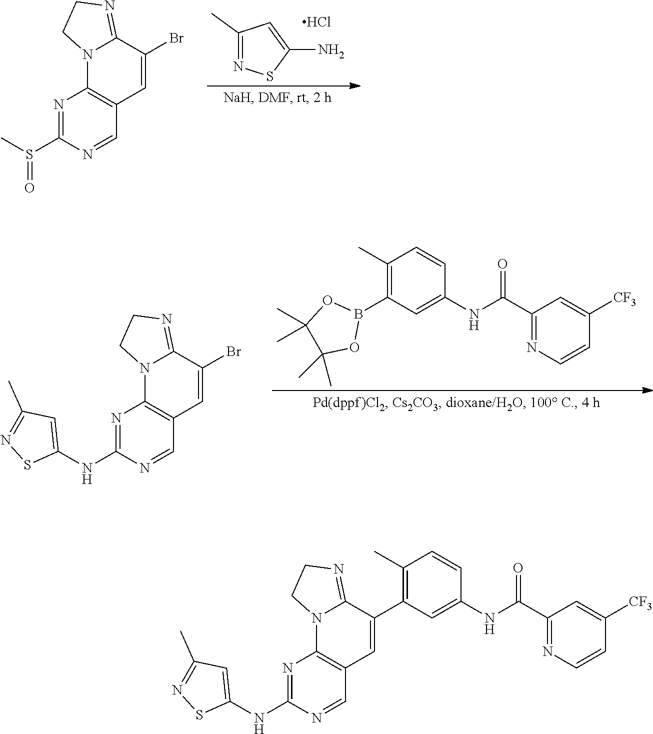

### Step 1: Synthesis of N-(6-bromo-8,9-dihydroimidazo[1′,2′:1,6]pyrido[2,3-d]pyrimidin-2-yl)-3-methylisothiazol-5-amine

To a solution of 3-methylisothiazol-5-amine hydrochloride (216 mg, 1.44 mmol) in DMF (18 mL) was added NaH (174 mg, 4.32 mmol). The reaction mixture was stirred at R.T under N2 for 1 hour. Then a solution of 6-bromo-2-(methylsulfinyl)-8,9-dihydroimidazo[1′,2′:1,6]pyrido[2,3-d]pyrimidine (450 mg, 1.44 mmol) in DMF (18 mL) was added dropwise to above reaction solution. The result solution was stirred at R.T under N2 for 1 hour. The solution was directly purified by flash chromatography (0.1%/HCl/CH3CN/H2O) to afford N-(6-bromo-8,9-dihydroimidazo[1′,2′:1,6]pyrido[2,3-d]pyrimidin-2-yl)-3-methylisothiazol-5-amine (170 mg, 29.7% yield). LCMS (M+H+) m/z: 363.0.

### Step 2: Synthesis of N-(4-methyl-3-(2-((3-methylisothiazol-5-yl)amino)-8,9-dihydroimidazo[1′,2′:1,6]pyrido[2,3-d]pyrimidin-6-yl)phenyl)-4-(trifluoromethyl)picolinamide hydrochloride

A mixture of N-(6-bromo-8,9-dihydroimidazo[1′,2′:1,6]pyrido[2,3-d]pyrimidin-2-yl)-3-methylisothiazol-5-amine (170 mg, 0.43 mmol), N-(4-methyl-3-(4,4,5,5-tetramethyl-1,3,2-dioxaborolan-2-yl)phenyl)-4-(trifluoromethyl)picolinamide (159 mg, 0.39 mmol), Cs2CO3 (419 mg, 1.29 mmol) and Pd(dppf)Cl2 (63 mg, 0.086 mmol) in dioxane (10 mL) and water (2.5 mL) was stirred at 100° C. under N2 for 2 hrs. The reaction mixture was cooled to r.t. and purified by prep-HPLC (0.1%/HCl/CH3CN/H2O) to afford N-(4-methyl-3-(2-((3-methylisothiazol-5-yl)amino)-8,9-dihydroimidazo[1′,2′:1,6]pyrido[2,3-d]pyrimidin-6-yl)phenyl)-4-(trifluoromethyl)picolinamide hydrochloride (121 mg, 47% yield) as a yellow solid.

¹H NMR (400 MHz, DMSO-d₆): δ 12.49 (s, 1H), 10.95 (s, 1H), 10.18 (s, 1H), 9.18 (s, 1H), 9.05 (d, J=4.8 Hz, 1H), 8.34 (s, 1H), 8.31 (s, 1H), 8.12 (d, J=4.8 Hz, 1H), 8.05 (d, J=2.0 Hz, 1H), 7.92 (dd, J=8.4, 2.0 Hz, 1H), 7.43 (d, J=8.4 Hz, 1H), 6.92 (s, 1H), 4.87 (t, J=10.0 Hz, 2H), 4.15 (t, J=10.0 Hz, 2H), 2.36 (s, 3H), 2.23 (s, 3H). LCMS (M+H⁺) m/z: 563.0.

### Example 243: Preparation of N-(3-(2-(isothiazol-4-ylamino)-8,9-dihydroimidazo[1′,2′:1,6]pyrido[2,3-d]pyrimidin-6-yl)-4-methylphenyl)-4-(trifluoromethyl)picolinamide (Compound 243)

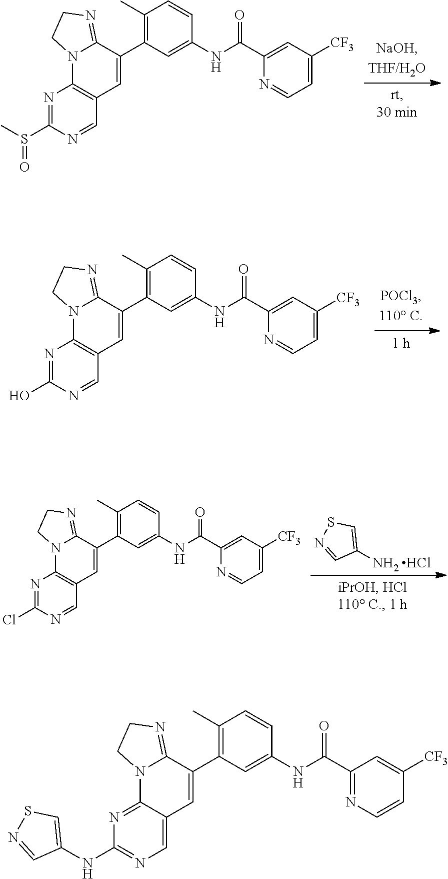

### Step 1: Synthesis of N-(3-(2-hydroxy-8,9-dihydroimidazo[1′,2′:1,6]pyrido[2,3-d]pyrimidin-6-yl)-4-methylphenyl)-4-(trifluoromethyl)picolinamide

To a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (2 g, crude, 3.9 mmol) in THF (20 mL) was added a solution of NaOH (11.7 mL, 11.7 mmol, 1M in water). The solution was stirred at rt for 0.5 h. The reaction mixture was purified by flash chromatography (0.1%/HCl/CH₃CN/H₂O) to afford N-(3-(2-hydroxy-8,9-dihydroimidazo[1′,2′:1,6]pyrido[2,3-d]pyrimidin-6-yl)-4-methylphenyl)-4-(trifluoromethyl)picolinamide (900 mg, 46% yield) as a yellow solid. LCMS (M+H⁺) m/z: 467.1

### Step 2: Synthesis of N-(3-(2-chloro-8,9-dihydroimidazo[1′,2′:1,6]pyrido[2,3-d]pyrimidin-6-yl)-4-methylphenyl)-4-(trifluoromethyl)picolinamide

A solution of N-(3-(2-hydroxy-8,9-dihydroimidazo[1′,2′:1,6]pyrido[2,3-d]pyrimidin-6-yl)-4-methylphenyl)-4-(trifluoromethyl)picolinamide (1.3 g, crude, 2.8 mmol) in POCl₃ (20 mL) was stirred at 110° C. for 2 hrs. The reaction mixture was cooled to r.t. and concentrated. The residue was purified by flash chromatography (0.1%/HCl/CH₃CN/H₂O) to afford N-(3-(2-chloro-8,9-dihydroimidazo[1′,2′:1,6]pyrido[2,3-d]pyrimidin-6-yl)-4-methylphenyl)-4-(trifluoromethyl)picolinamide (220 mg, 16% yield) as a yellow solid. LCMS (M+H⁺) m/z: 485.2

### Step 3: Synthesis of N-(3-(2-(isothiazol-4-ylamino)-8,9-dihydroimidazo[1′,2′:1,6]pyrido[2,3-d]pyrimidin-6-yl)-4-methylphenyl)-4-(trifluoromethyl)picolinamide hydrochloride

A solution of N-(3-(2-chloro-8,9-dihydroimidazo[1′,2′:1,6]pyrido[2,3-d]pyrimidin-6-yl)-4-methylphenyl)-4-(trifluoromethyl)picolinamide (200 mg, 0.412 mmol), isothiazol-4-amine hydrochloride (112 mg, 0.824 mmol) in i-PrOH (32 mL) and HCl/dioxane (8 drops, 4 M in dioxane) was stirred at 110° C. in a sealed tube for 2 h. The reaction mixture was cooled to r.t. and concentrated. The residue was purified by prep-HPLC (0.1%/HCl/CH₃CN/H₂O) to afford N-(3-(2-(isothiazol-4-ylamino)-8,9-dihydroimidazo[1′,2′:1,6]pyrido[2,3-d]pyrimidin-6-yl)-4-methylphenyl)-4-(trifluoromethyl)picolinamide hydrochloride (107 mg, 44% yield) as a yellow solid.

¹H NMR (400 MHz, DMSO-d6) δ 11.38 (s, 1H), 10.94 (s, 1H), 10.05 (s, 1H), 9.20 (s, 1H), 9.12 (s, 1H), 9.05 (d, J=4.8 Hz, 1H), 8.82 (s, 1H), 8.34 (s, 1H), 8.27 (s, 1H), 8.12 (d, J=4.4 Hz, 1H), 8.04 (d, J=1.6 Hz, 1H), 7.92 (d, J=8.4 Hz, 1H), 7.43 (d, J=8.4 Hz, 1H), 4.84-4.80 (m, 2H), 4.14-4.10 (m, 2H), 2.23 (s, 3H). LCMS (M+H⁺) m/z: 549.1

### Example 244: Preparation of N-(4-methyl-3-(8-((1-methyl-1H-imidazol-2-yl)amino)-1,2-dihydroimidazo[1,2-a][1,6]naphthyridin-4-yl)phenyl)-4-(trifluoromethyl)picolinamide (Compound 244)

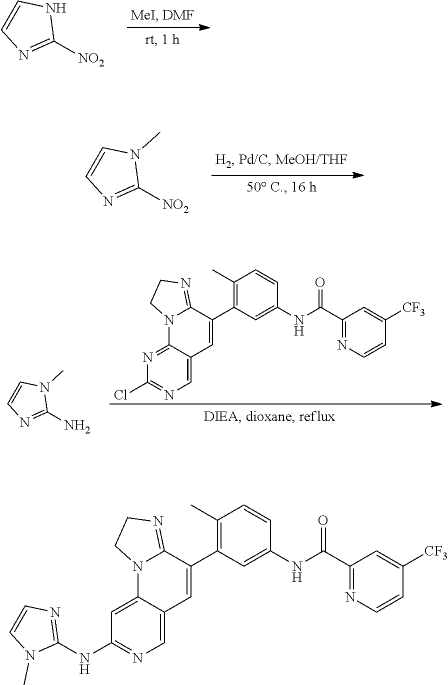

### Step 1: Synthesis of 1-methyl-2-nitro-1H-imidazole

To a solution of 2-nitro-1H-imidazole (1 g, 8.84 mmol) in DMF (40 mL) was added NaH (707 mg, 17.68 mmol, 60% wt in mineral oil). The mixture was stirred at r.t under N2 for 1 hr. Then iodomethane (1.95 g, 13.26 mmol) was added. The mixture was stirred at r.t under N2 for 15 mins. The reaction mixture was quenched with sat. NH₄Cl (100 mL) and water (100 mL). The reaction mixture was extracted with EA (100 mL×2). The combined organic phase was concentrated. The residue was purified by flash chromatography to afford 1-methyl-2-nitro-1H-imidazole (600 mg, 53% yield) as a yellow solid. LCMS (M+H⁺) m/z: 128.0.

### Step 2: Synthesis of 1-methyl-1H-imidazol-2-amine

To a solution of 1-methyl-2-nitro-1H-imidazole (620 mg, 4.88 mmol) in MeOH (20 mL) was added Pd/C (124 mg, 20% wt). The mixture was stirred at 50° C. under H2 balloon for 6 hrs. The mixture was filtered and the filtrate was concentrated to afford 1-methyl-1H-imidazol-2-amine (400 mg, 84% yield) as a brown solid. ¹H NMR (400 MHz, DMSO-d₆) δ 6.45 (d, J=1.2 Hz, 1H), 6.31 (d, J=1.2 Hz, 1H), 5.21 (s, 2H), 3.29 (s, 3H).

### Step 3: Synthesis of N-(4-methyl-3-(8-((1-methyl-1H-imidazol-2-yl)amino)-1,2-dihydroimidazo[1,2-a][1,6]naphthyridin-4-yl)phenyl)-4-(trifluoromethyl)picolinamide

A solution of N-(3-(2-chloro-8,9-dihydroimidazo[1′,2′:1,6]pyrido[2,3-d]pyrimidin-6-yl)-4-methylphenyl)-4-(trifluoromethyl)picolinamide (50 mg, 0.103 mmol), 1-methyl-1H-imidazol-2-amine (30 mg, 0.309 mmol) and DIEA (133 mg, 1.03 mmol) in dioxane (4 mL) was stirred at 110° C. for 8 hrs. The reaction mixture was cooled to r.t. and concentrated. The residue was purified by prep-HPLC (0.1%/TFA/CH₃CN/H₂O) to afford N-(4-methyl-3-(8-((1-methyl-1H-imidazol-2-yl)amino)-1,2-dihydroimidazo[1,2-a][1,6]naphthyridin-4-yl)phenyl)-4-(trifluoromethyl)picolinamide TFA salt (16 mg, 20% yield) as a yellow solid. ¹H NMR (400 MHz, DMSO-d₆) δ 12.22 (s, 1H), 10.96 (s, 1H), 10.22 (s, 1H), 9.18 (s, 1H), 9.06 (d, J=5.2 Hz, 1H), 8.34 (s, 1H), 8.31 (s, 1H), 8.12 (dd, J=5.0, 1.2 Hz, 1H), 8.07 (d, J=2.0 Hz, 1H), 7.93 (dd, J=8.0, 2.0 Hz, 1H), 7.44 (d, J=8.4 Hz, 1H), 6.45 (s, 1H), 4.80 (t, J=9.6 Hz, 2H), 4.14 (t, J=10.4 Hz, 2H), 2.27 (s, 3H), 2.23 (s, 3H). LCMS (M+H⁺) m/z: 546.1.

### Example 245: Preparation of N-(3-(2-(isoxazol-4-ylamino)-8,9-dihydroimidazo[1′,2′:1,6]pyrido[2,3-d]pyrimidin-6-yl)-4-methylphenyl)-4-(trifluoromethyl)picolinamide (Compound 245)

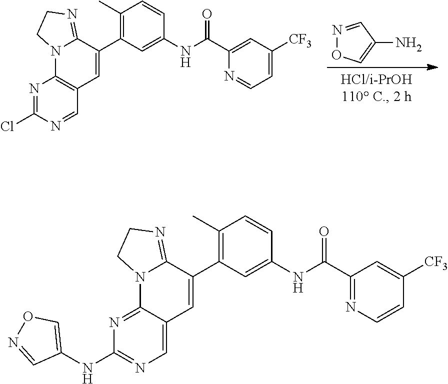 [SMILES: Cc1ccc(NC(=O)c2cc(C(F)(F)F)ccn2)cc1C1=Cc2cnc(Cl)nc2N2CCN=C12 ; Nc1cnoc1 ; Cc1ccc(NC(=O)c2cc(C(F)(F)F)ccn2)cc1C1=Cc2cnc(Nc3cnoc3)nc2N2CCN=C12]

### Step 1: Synthesis of N-(3-(2-(isoxazol-4-ylamino)-8,9-dihydroimidazo[1′,2′:1,6]pyrido[2,3-d]pyrimidin-6-yl)-4-methylphenyl)-4-(trifluoromethyl)picolinamide hydrochloride

A solution of N-(3-(2-chloro-8,9-dihydroimidazo[1′,2′:1,6]pyrido[2,3-d]pyrimidin-6-yl)-4-methylphenyl)-4-(trifluoromethyl)picolinamide (240 mg, 0.494 mmol), isoxazol-4-amine (166 mg, 1.976 mmol) in i-PrOH (40 mL) and HCl/dioxane (6 drops, 4 M in dioxane) was stirred at 110° C. in a sealed tube for 2 h. The reaction mixture was cooled to r.t. and concentrated. The residue was purified by Prep-HPLC (0.1%/HCl/CH₃CN/H₂O) to afford N-(3-(2-(isoxazol-4-ylamino)-8,9-dihydroimidazo[1′,2′:1,6]pyrido[2,3-d]pyrimidin-6-yl)-4-methylphenyl)-4-(trifluoromethyl)picolinamide (106 mg, 38.3% yield) as a yellow solid.

¹H NMR (400 MHz, DMSO-d₆): δ 10.96 (s, 1H), 10.93 (s, 1H), 10.05 (s, 1H), 9.33 (s, 1H), 9.09 (s, 1H), 9.05 (d, J=4.8 Hz, 1H), 8.80 (s, 1H), 8.33 (s, 1H), 8.27 (s, 1H), 8.12 (t, J=5.2 Hz, 1H), 8.04 (d, J=2.4 Hz, 1H), 7.92 (dd, J=8.4, 2.4 Hz, 1H), 7.43 (d, J=8.4 Hz, 1H), 4.87 (t, J=10.0 Hz, 2H), 4.10 (t, J=10.0 Hz, 2H), 2.22 (s, 3H). LCMS (M+H⁺) m/z: 533.5.

### Example 246: Preparation of N-(4-methyl-3-(2-((3-methylisoxazol-5-yl)amino)-8,9-dihydroimidazo[1′,2′:1,6]pyrido[2,3-d]pyrimidin-6-yl)phenyl)-4-(trifluoromethyl)picolinamide

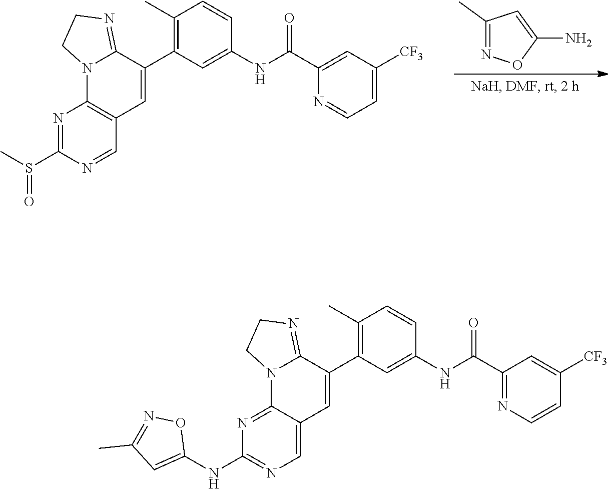

### Step 1: Synthesis of N-(4-methyl-3-(2-((3-methylisoxazol-5-yl)amino)-8,9-dihydroimidazo[1′,2′:1,6]pyrido[2,3-d]pyrimidin-6-yl)phenyl)-4-(trifluoromethyl)picolinamide

To a solution of 3-methylisoxazol-5-amine (133 mg, 1.36 mmol) in DMF (6 mL) was added NaH (109 mg, 2.72 mmol). The reaction mixture was stirred at rt under N₂ for 1 h. Then a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (700 mg crude, 1.36 mmol) in DMF (12 mL) was added dropwise to above reaction solution. The result solution was stirred at rt under N₂ for 1 h, quenched with sat. NH₄Cl (10 mL) and water (50 mL). The reaction mixture was extracted with EA (30 mL×2). The organic phase was concentrated in vacuum. The crude product was purified by prep-HPLC (0.1%/TFA/CH₃CN/H₂O) to afford N-(4-methyl-3-(2-((3-methylisoxazol-5-yl)amino)-8,9-dihydroimidazo[1′,2′:1,6]pyrido[2,3-d]pyrimidin-6-yl)phenyl)-4-(trifluoromethyl)picolinamide TFA salt (6.5 mg, 0.7% yield) as a yellow solid. ¹H NMR (400 MHz, DMSO-d₆) δ 12.22 (s, 1H), 10.96 (s, 1H), 10.22 (s, 1H), 9.18 (s, 1H), 9.06 (d, J=5.2 Hz, 1H), 8.34 (s, 1H), 8.31 (s, 1H), 8.12 (dd, J=5.0, 1.2 Hz, 1H), 8.07 (d, J=2.0 Hz, 1H), 7.93 (dd, J=8.0, 2.0 Hz, 1H), 7.44 (d, J=8.4 Hz, 1H), 6.45 (s, 1H), 4.80 (t, J=9.6 Hz, 2H), 4.14 (t, J=10.4 Hz, 2H), 2.27 (s, 3H), 2.23 (s, 3H). LCMS (M+H⁺) m/z: 547.2.

### Example 247: Preparation of N-(4-methyl-3-(2-((5-methyl-1,3,4-thiadiazol-2-yl)amino)-8,9-dihydroimidazo[1′,2′:1,6]pyrido[2,3-d]pyrimidin-6-yl)phenyl)-4-(trifluoromethyl)picolinamide (Compound 247)

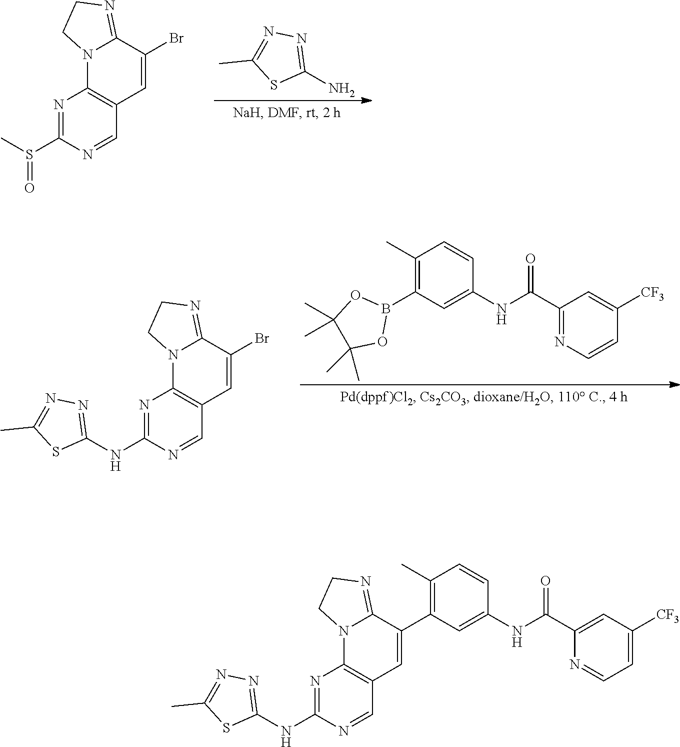

### Step 1: Synthesis of N-(6-bromo-8,9-dihydroimidazo[1′,2′:1,6]pyrido[2,3-d]pyrimidin-2-yl)-5-methyl-1,3,4-thiadiazol-2-amine

To a solution of 5-methyl-1,3,4-thiadiazol-2-amine (111 mg, 0.96 mmol) in DMF (4 mL) was added NaH (38 mg, 0.96 mmol). The reaction mixture was stirred at R.T under N2 for 0.5 hour. Then a solution of 6-bromo-2-(methylsulfinyl)-8,9-dihydroimidazo[1′,2′:1,6]pyrido[2,3-d]pyrimidine (150 mg, 0.48 mmol) in DMF (18 mL) was added dropwise to above reaction solution. The result solution was stirred at R.T under N2 for 1 h. The solution was quenched with sat. NH4Cl. Water was added, the reaction mixture was extracted with EA. The combined extracts were washed with brine. Concentration and purification by flash chromatography afforded N-(6-bromo-8,9-dihydroimidazo[1′,2′:1,6]pyrido[2,3-d]pyrimidin-2-yl)-5-methyl-1,3,4-thiadiazol-2-amine (24 mg, 14% yield) as a yellow solid. LCMS (M+H⁺) m/z: 366.0

### Step 2: Synthesis of N-(4-methyl-3-(2-((5-methyl-1,3,4-thiadiazol-2-yl)amino)-8,9-dihydroimidazo[1′,2′:1,6]pyrido[2,3-d]pyrimidin-6-yl)phenyl)-4-(trifluoromethyl)picolinamide

A mixture of N-(6-bromo-8,9-dihydroimidazo[1′,2′:1,6]pyrido[2,3-d]pyrimidin-2-yl)-5-methyl-1,3,4-thiadiazol-2-amine (24 mg, 0.066 mmol), N-(4-methyl-3-(4,4,5,5-tetramethyl-1,3,2-dioxaborolan-2-yl)phenyl)-4-(trifluoromethyl)picolinamide (35 mg, 0.0858 mmol), Cs2CO3 (64 mg, 0.198 mmol) and Pd(dppf)Cl₂ (10 mg, 0.0132 mmol) in dioxane (6 mL) and water (2 mL) was stirred at 100° C. under N2 for 4 h. The reaction mixture was cooled to r.t. concentrated. The residue was purified by column chromatography (DCM/MeOH=10/1) to give crude product, which was purified by prep-HPLC (0.1%/HCl/CH3CN/H2O) to afford N-(4-methyl-3-(2-((5-methyl-1,3,4-thiadiazol-2-yl)amino)-8,9-dihydroimidazo[1′,2′:1,6]pyrido[2,3-d]pyrimidin-6-yl)phenyl)-4-(trifluoromethyl)picolinamide

(2.3 mg, 6% yield) as a yellow solid. ¹H NMR (400 MHz, DMSO-d₆): δ 10.95 (s, 1H), 10.26 (s, 1H), 9.21 (s, 1H), 9.05 (d, J=4.4 Hz, 1H), 8.33 (s, 1H), 8.32 (s, 1H), 8.11 (d, J=4.4 Hz, 1H), 8.05 (d, J=2.0 Hz, 1H), 7.91 (dd, J=8.4, 2.4 Hz, 1H), 7.43 (d, J=8.4 Hz, 1H), 4.84-4.79 (m, 2H), 4.17-4.11 (m, 2H), 2.68 (s, 3H), 2.22 (s, 3H). LCMS (M+H⁺) m/z: 564.1.

### Example 248: Preparation of N-(3-(2-((1,3,4-thiadiazol-2-yl)amino)-8,9-dihydroimidazo[1′,2′:1,6]pyrido[2,3-d]pyrimidin-6-yl)-4-methylphenyl)-4-(trifluoromethyl)picolinamide (Compound 248)

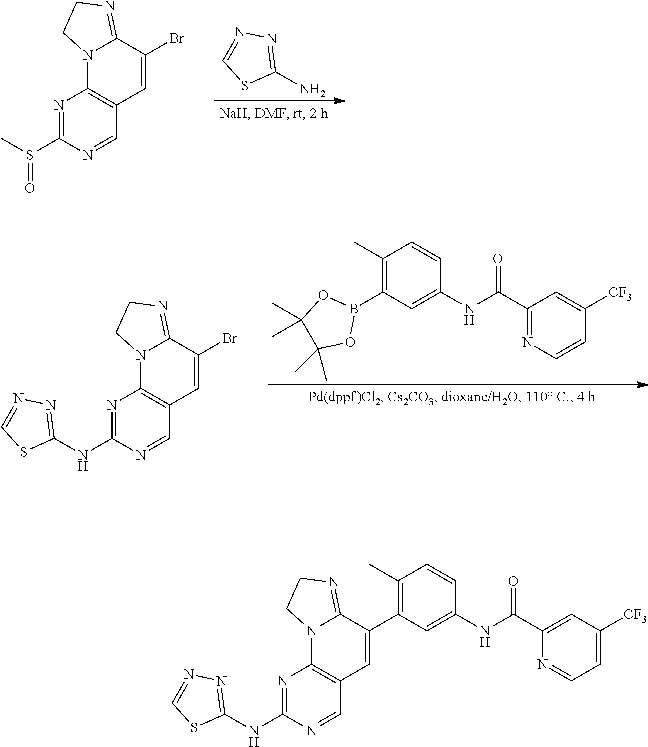

### Step 1: Synthesis of N-(6-bromo-8,9-dihydroimidazo[1′,2′:1,6]pyrido[2,3-d]pyrimidin-2-yl)-1,3,4-thiadiazol-2-amine

To a solution of 1,3,4-thiadiazol-2-amine (162 mg, 1.6 mmol) in DMF (4 mL) was added NaH (64 mg, 1.6 mmol). The reaction mixture was stirred at R.T under N₂ for 0.5 hour. Then a solution of 6-bromo-2-(methylsulfinyl)-8,9-dihydroimidazo[1′,2′:1,6]pyrido[2,3-d]pyrimidine (250 mg, 0.80 mmol) in DMF (24 mL) was added dropwise to above reaction solution. The result solution was stirred at R.T under N₂ for 1 hour. The solution was quenched with sat. NH4Cl. Water was added, the reaction mixture was extracted with EA. The combined extracts were washed with brine. Concentration and purification by flash chromatography afforded N-(6-bromo-8,9-dihydroimidazo[1′,2′:1,6]pyrido[2,3-d]pyrimidin-2-yl)-1,3,4-thiadiazol-2-amine (33 mg, 14% yield) as a yellow solid. LCMS (M+H⁺) m/z: 352.0

### Step 2: Synthesis of N-(3-(2-((1,3,4-thiadiazol-2-yl)amino)-8,9-dihydroimidazo[1′,2′:1,6]pyrido[2,3-d]pyrimidin-6-yl)-4-methylphenyl)-4-(trifluoromethyl)picolinamide hydrochloride

A mixture of N-(6-bromo-8,9-dihydroimidazo[1′,2′:1,6]pyrido[2,3-d]pyrimidin-2-yl)-1,3,4-thiadiazol-2-amine (33 mg, 0.09 mmol), N-(4-methyl-3-(4,4,5,5-tetramethyl-1,3,2-dioxaborolan-2-yl)phenyl)-4-(trifluoromethyl)picolinamide (47 mg, 0.117 mmol), Cs2CO3 (87 mg, 0.27 mmol) and Pd(dppf)Cl2 (13 mg, 0.018 mmol) in dioxane (6 mL) and water (2 mL) was stirred at 100° C. under N2 for 4 h. The reaction mixture was cooled to r.t. concentrated. The residue was purified by flash chromatography to give crude product, which was purified by Prep-HPLC (0.10%/HCl/CH3CN/H2O) to afford N-(3-(2-((1,3,4-thiadiazol-2-yl)amino)-8,9-dihydroimidazo[1′,2′:1,6]pyrido[2,3-d]pyrimidin-6-yl)-4-methylphenyl)-4-(trifluoromethyl)picolinamide hydrochloride (6.4 mg, 12% yield) as a yellow solid. 1H NMR (400 MHz, DMSO-d6): δ 10.95 (s, 1H), 10.32 (s, 1H), 9.30 (s, 1H), 9.24 (s, 1H), 9.05 (d, J=5.2 Hz, 1H), 8.34 (s, 1H), 8.33 (s, 1H), 8.11 (d, J=4.4 Hz, 1H), 8.06 (s, 1H), 7.92 (d, J=8.4 Hz, 1H), 7.43 (d, J=8.0 Hz, 1H), 4.83 (t, J=10.0 Hz, 2H), 4.15 (t, J=10.0 Hz, 2H), 2.22 (s, 3H). LCMS (M+H+) m/z: 550.1.

### Example 249: Preparation of N-(3-(2-((1,2,4-thiadiazol-5-yl)amino)-8,9-dihydroimidazo[1′,2′:1,6]pyrido[2,3-d]pyrimidin-6-yl)-4-methylphenyl)-4-(trifluoromethyl)picolinamide (Compound 249)

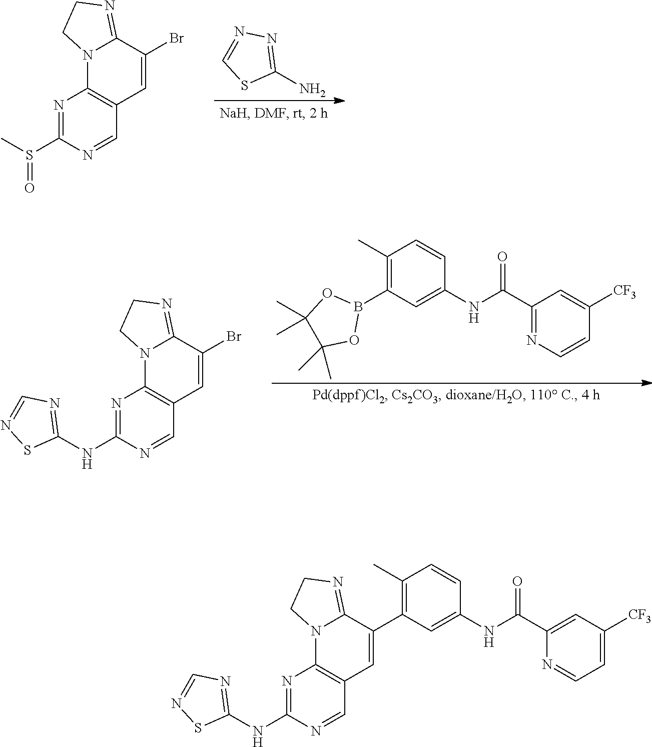

### Step 1: Synthesis of N-(6-bromo-8,9-dihydroimidazo[1′,2′:1,6]pyrido[2,3-d]pyrimidin-2-yl)-1,2,4-thiadiazol-5-amine

To a solution of 1,2,4-thiadiazol-2-amine (65 mg, 0.64 mmol) in DMF (2 mL) was added NaH (26 mg, 0.64 mmol). The reaction mixture was stirred at R.T under N2 for 0.5 hour. Then a solution of 6-bromo-2-(methylsulfinyl)-8,9-dihydroimidazo[1′,2′:1,6]pyrido[2,3-d]pyrimidine (100 mg, 0.32 mmol) in DMF (12 mL) was added dropwise to above reaction solution. The result solution was stirred at R.T under N2 for 1 hour. The solution was quenched with sat. NH4Cl. Water was added, the reaction mixture was extracted with EA. The combined extracts were washed with brine. Concentration and purification by flash chromatography afforded N-(6-bromo-8,9-dihydroimidazo[1′,2′:1,6]pyrido[2,3-d]pyrimidin-2-yl)-1,2,4-thiadiazol-5-amine (30 mg, 27% yield) as a yellow solid. LCMS (M+H⁺) m/z: 351.9

### Step 2: Synthesis of N-(3-(2-((1,2,4-thiadiazol-5-yl)amino)-8,9-dihydroimidazo[1′,2′:1,6]pyrido[2,3-d]pyrimidin-6-yl)-4-methylphenyl)-4-(trifluoromethyl)picolinamide

A mixture of N-(6-bromo-8,9-dihydroimidazo[1′,2′:1,6]pyrido[2,3-d]pyrimidin-2-yl)-1,2,4-thiadiazol-5-amine (30 mg, 0.086 mmol), N-(4-methyl-3-(4,4,5,5-tetramethyl-1,3,2-dioxaborolan-2-yl)phenyl)-4-(trifluoromethyl)picolinamide (52 mg, 0.129 mmol), Cs2CO3 (84 mg, 0.258 mmol) and Pd(dppf)Cl2 (13 mg, 0.0172 mmol) in dioxane (6 mL) and water (2 mL) was stirred at 100° C. under N2 for 4 h. The reaction mixture was cooled to r.t. concentrated. The residue was purified by column DCM/MeOH=10/1 to give crude product, which was purified by Prep-HPLC (0.1%/HCl/CH3CN/H2O) to afford N-(3-(2-((1,2,4-thiadiazol-5-yl)amino)-8,9-dihydroimidazo[1′,2′:1,6]pyrido[2,3-d]pyrimidin-6-yl)-4-methylphenyl)-4-(trifluoromethyl)picolinamide (13 mg, 27% yield) as a yellow solid.

¹H NMR (400 MHz, DMSO-d₆): δ 13.52 (br, 1H), 10.96 (s, 1H), 10.41 (s, 1H), 9.31 (s, 1H), 9.06 (d, J=5.2 Hz, 1H), 8.55 (s, 1H), 8.39 (s, 1H), 8.33 (s, 1H), 8.12 (dd, J=5.2, 1.2 Hz, 1H), 8.07 (d, J=2.0 Hz, 1H), 7.93 (dd, J=8.4, 2.0 Hz, 1H), 7.44 (d, J=8.4 Hz, 1H), 4.93-4.90 (m, 2H), 4.21-4.16 (m, 2H), 2.23 (s, 3H). LCMS (M+H⁺) m/z: 550.2.

### Example 250: Preparation of N-(3-(2-((1,2,3-thiadiazol-5-yl)amino)-8,9-dihydroimidazo[1′,2′:1,6]pyrido[2,3-d]pyrimidin-6-yl)-4-methylphenyl)-4-(trifluoromethyl)picolinamide (Compound 250)

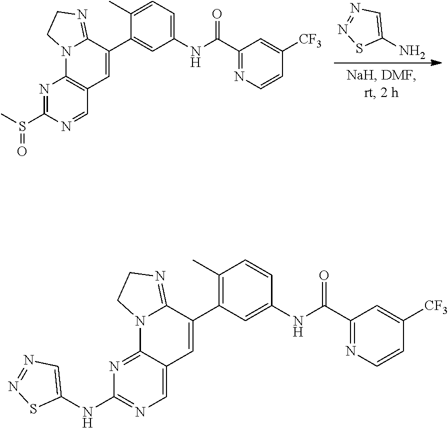 [SMILES: Cc1ccc(NC(=O)c2cc(C(F)(F)F)ccn2)cc1C1=Cc2cnc(S(C)=O)nc2N2CCN=C12 ; Nc1cnns1 ; Cc1ccc(NC(=O)c2cc(C(F)(F)F)ccn2)cc1C1=Cc2cnc(Nc3cnns3)nc2N2CCN=C12]

### Step 1: Synthesis of N-(3-(2-((1,2,3-thiadiazol-5-yl)amino)-8,9-dihydroimidazo[1′,2′:1,6]pyrido[2,3-d]pyrimidin-6-yl)-4-methylphenyl)-4-(trifluoromethyl)picolinamide hydrochloride

To a solution of 1,2,3-thiadiazol-5-amine (47 mg, 0.465 mmol) in DMF (4 mL) was added NaH (37 mg, 0.93 mmol, 60% wt in mineral oil). The reaction mixture was stirred at R.T under N₂ for 1 hour. Then a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (160 mg, 0.31 mmol) in DMF (6 mL) was dropwised to above reaction solution. The reaction mixture was stirred at R.T under N₂ for 1 hour. The result solution was directly purified by flash chromatography to get crude product. The crude product was purified by Prep-HPLC (0.1%/HCl/CH3CN/H2O) to afford N-(3-(2-((1,2,3-thiadiazol-5-yl)amino)-8,9-dihydroimidazo[1′,2′:1,6]pyrido[2,3-d]pyrimidin-6-yl)-4-methylphenyl)-4-(trifluoromethyl)picolinamide (5.1 mg, 3% yield) as a yellow solid.

¹H NMR (400 MHz, DMSO-d₆): δ 10.95 (s, 1H), 10.51 (s, 1H), 9.16 (s, 1H), 9.05 (d, J=5.2 Hz, 1H), 8.34 (d, J=5.6 Hz, 2H), 8.12 (dd, J=5.2, 1.2 Hz, 1H), 8.05 (d, J=2.0 Hz, 1H), 7.92 (dd, J=8.4, 2.4 Hz, 1H), 7.43 (d, J=8.4 Hz, 1H), 4.50 (t, J=10.0 Hz, 2H), 4.07 (t, J=10.0 Hz, 2H), 2.21 (s, 3H). LCMS (M+H⁺) m/z: 550.2.

### Example 251: Preparation of N-(3-(2-((1,3,4-oxadiazol-2-yl)amino)-8,9-dihydroimidazo[1′,2′:1,6]pyrido[2,3-d]pyrimidin-6-yl)-4-methylphenyl)-4-(trifluoromethyl)picolinamide (Compound 251)

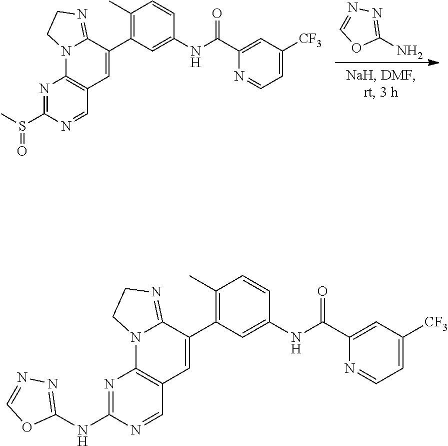 [SMILES: Cc1ccc(NC(=O)c2cc(C(F)(F)F)ccn2)cc1C1=Cc2cnc(S(C)=O)nc2N2CCN=C12 ; Nc1nnco1 ; Cc1ccc(NC(=O)c2cc(C(F)(F)F)ccn2)cc1C1=Cc2cnc(Nc3nnco3)nc2N2CCN=C12]

### Step 1: Synthesis of N-(3-(2-((1,3,4-oxadiazol-2-yl)amino)-8,9-dihydroimidazo[1′,2′:1,6]pyrido[2,3-d]pyrimidin-6-yl)-4-methylphenyl)-4-(trifluoromethyl)picolinamide 2,2,2-trifluoroacetate

To a solution of 1,3,4-oxadiazol-2-amine (45 mg, 0.528 mmol) in DMF (3 mL) was added NaH (21 mg, 0.528 mmol). The reaction mixture was stirred at R.T under N2 for 1 hour. Then a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (90 mg, 0.176 mmol) in DMF (8 mL) was added dropwise to above reaction solution. The result solution was stirred at R.T under N₂ for 1 hour and quenched with sat. NH₄Cl (10 mL) and water (60 mL). The reaction mixture was extracted with EA (40 mL×3). The organic phase was concentrated and the crude product was purified by Prep-HPLC (0.1%/TFA/CH₃CN/H₂O) to afford N-(3-(2-((1,3,4-oxadiazol-2-yl)amino)-8,9-dihydroimidazo[1′,2′:1,6]pyrido[2,3-d]pyrimidin-6-yl)-4-methylphenyl)-4-(trifluoromethyl)picolinamide 2,2,2-trifluoroacetate (15 mg, 13% yield) as a yellow solid.

¹H NMR (400 MHz, DMSO-d₆): δ 12.19 (br, 1H), 10.95 (s, 1H), 10.27 (br s, 1H), 9.18 (s, 1H), 9.11 (s, 1H), 9.06 (d, J=4.8 Hz, 1H), 8.34 (s, 1H), 8.31 (s, 1H), 8.12 (dd, J=5.2, 1.2 Hz, 1H), 8.06 (d, J=2.0 Hz, 1H), 7.92 (dd, J=8.4, 2.0 Hz, 1H), 7.44 (d, J=8.4 Hz, 1H), 4.68 (t, J=10.0 Hz, 2H), 4.09 (t, J=10.0 Hz, 2H), 2.23 (s, 3H). LCMS (M+H⁺) m/z: 534.1.

### Example 252: Preparation of N-(4-methyl-3-(2-((5-methyl-1,3,4-oxadiazol-2-yl)amino)-8,9-dihydroimidazo[1′,2′:1,6]pyrido[2,3-d]pyrimidin-6-yl)phenyl)-4-(trifluoromethyl)picolinamide (Compound 252)

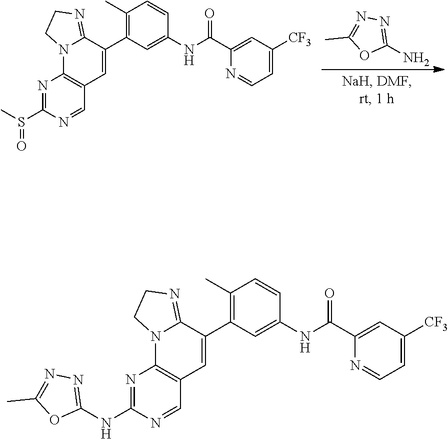

### Step 1: Synthesis of N-(4-methyl-3-(2-((5-methyl-1,3,4-oxadiazol-2-yl)amino)-8,9-dihydroimidazo[1′,2′:1,6]pyrido[2,3-d]pyrimidin-6-yl)phenyl)-4-(trifluoromethyl)picolinamide formate

To a solution of 5-methyl-1,3,4-oxadiazol-2-amine (52 mg, 0.528 mmol) in DMF (3 mL) was added NaH (21 mg, 0.528 mmol). The reaction mixture was stirred at rt under N2 for 1 hour. Then a solution of N-(4-methyl-3-(2-(methylsulfinyl)-8,9-dihydroimidazo[1′,2′:1,6]pyrido[2,3-d]pyrimidin-6-yl)phenyl)-4-(trifluoromethyl)picolinamide (90 mg, 0.176 mmol) in DMF (8 mL) was added dropwise to above reaction solution. The result solution was stirred at R.T under N2 for 1 hour and quenched with sat. NH4Cl (10 mL) and water (60 mL). The reaction mixture was extracted with EA (40 mL×3). The organic phase was concentrated and the crude product was purified by Prep-HPLC (0.10%/FA/CH3CN/H2O) to afford N-(4-methyl-3-(2-((5-methyl-1,3,4-oxadiazol-2-yl)amino)-8,9-dihydroimidazo[1′,2′:1,6]pyrido[2,3-d]pyrimidin-6-yl)phenyl)-4-(trifluoromethyl)picolinamide formate (7.5 mg, 7% yield) as a yellow solid. ¹H NMR (400 MHz, DMSO-d₆): δ 10.78 (s, 1H), 9.04 (d, J=4.8 Hz, 1H), 8.40 (s, 1H), 8.34 (s, 1H), 8.10 (d, J=4.8 Hz, 1H), 7.87 (d, J=2.0 Hz, 1H), 7.78 (dd, J=8.4, 2.0 Hz, 1H), 7.27-7.25 (m, 2H), 4.09-4.05 (m, 2H), 3.98-3.94 (m, 2H), 2.47 (s, 3H), 2.23 (s, 3H). LCMS (M+H⁺) m/z: 548.2.

### Example 253 and 254: Preparation of N-(3-(2-((1H-1,2,4-triazol-3-yl)amino)-8,9-dihydroimidazo[1′,2′:1,6]pyrido[2,3-d]pyrimidin-6-yl)-4-methylphenyl)-4-(trifluoromethyl)picolinamide (Compound 253) and N-(3-(2-(3-amino-4H-1,2,4-triazol-4-yl)-8,9-dihydroimidazo[1′,2′:1,6]pyrido[2,3-d]pyrimidin-6-yl)-4-methylphenyl)-4-(trifluoromethyl)picolinamide (Compound 254)

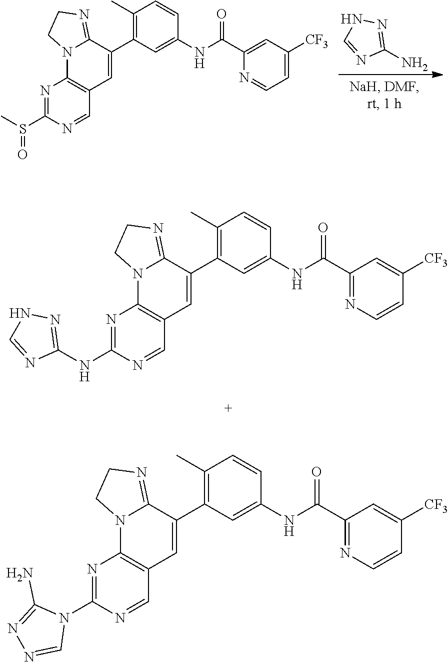

The preparation was similar to Example 252 using 1H-1,2,4-triazol-3-amine as starting material. Two isomer were obtained, Compound 253 (4.4 mg, 4.8% yield) and Compound 254 (2.1 mg, 2.3% yield) as a yellow solid. LCMS (M+H⁺) m/z: 530.3.

Compound 253 ¹H NMR (400 MHz, DMSO-d₆): δ 10.96 (s, 1H), 10.66 (s, 1H), 9.39 (s, 1H), 9.05 (d, J=5.2 Hz, 1H), 8.49 (s, 1H), 8.33 (s, 1H), 8.11 (dd, J=5.2, 1.2 Hz, 1H), 8.08 (d, J=2.4 Hz, 1H), 8.04 (br s, 1H), 7.93 (dd, J=8.4, 2.4 Hz, 1H), 7.81 (s, 1H), 7.44 (d, J=8.8 Hz, 1H), 4.81 (t, J=10.0 Hz, 2H), 4.15 (t, J=10.0 Hz, 2H), 2.24 (s, 3H). LCMS (M+H⁺) m/z: 533.3.

Compound 254 ¹H NMR (400 MHz, DMSO-d₆): δ 10.96 (s, 1H), 10.56 (s, 1H), 9.33 (s, 1H), 9.26 (s, 1H), 9.05 (d, J=4.8 Hz, 1H), 8.40 (s, 1H), 8.33 (s, 1H), 8.12-8.07 (m, 2H), 7.92 (dd, J=8.4, 2.4 Hz, 1H), 7.44 (d, J=8.4 Hz, 1H), 6.29 (br, 1H), 4.75 (t, J=10.0 Hz, 2H), 4.15 (t, J=10.0 Hz, 2H), 2.23 (s, 3H). LCMS (M+H⁺) m/z: 533.3.

### Example 255: Preparation of N-(4-methyl-3-(2-(thiazol-2-ylamino)-8,9-dihydroimidazo[1′,2′:1,6]pyrido[2,3-d]pyrimidin-6-yl)phenyl)-4-(trifluoromethyl)picolinamide (Compound 255)

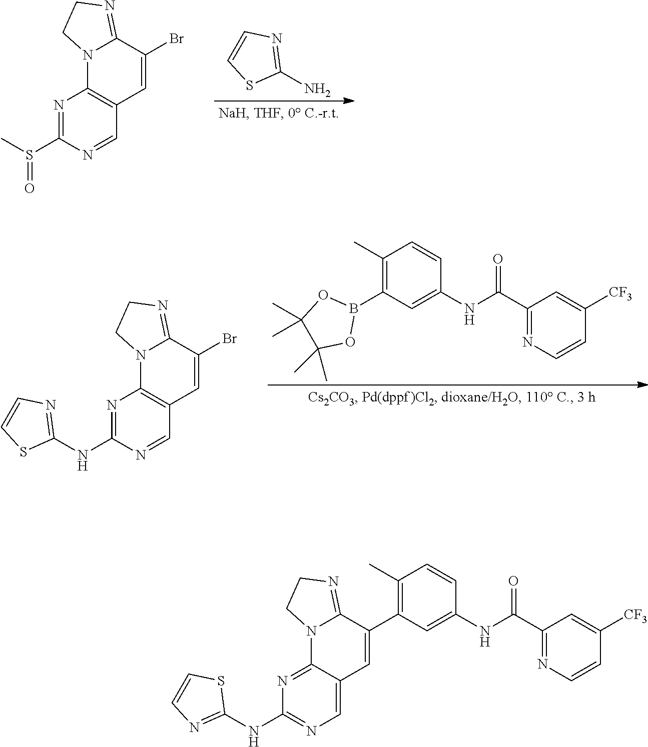

### Step 1: Synthesis of N-(6-bromo-8,9-dihydroimidazo[1′,2′:1,6]pyrido[2,3-d]pyrimidin-2-yl)thiazol-2-amine

To a solution of thiazol-2-amine (95.8 mg, 0.96 mmol) in THF (1 mL) was added NaH (46 mg, 1.92 mmol) at 0° C. and the mixture was stirred at 0° C. for 1 h, 6-bromo-2-(methylsulfinyl)-8,9-dihydroimidazo[1′,2′:1,6]pyrido[2,3-d]pyrimidine (200 mg, 0.64 mmol) was added to the mixture. The resulting mixture was stirred for 2 h at r.t. H₂O (30 mL) was added, the reaction mixture was extracted with DCM (30 mL×3). The combined organic phases were washed with brine (60 mL), dried over Na2SO4, filtered and concentrated under vacuum to afford N-(6-bromo-8,9-dihydroimidazo[1′,2′:1,6]pyrido[2,3-d]pyrimidin-2-yl)thiazol-2-amine (70 mg, 31% yield) as a brown solid. LCMS (M+H⁺) m/z: 349.0 and 351.0.

### Step 2: Synthesis of N-(4-methyl-3-(2-(thiazol-2-ylamino)-8,9-dihydroimidazo[1′,2′:1,6]pyrido[2,3-d]pyrimidin-6-yl)phenyl)-4-(trifluoromethyl)picolinamide

A mixture of N-(6-bromo-8,9-dihydroimidazo[1′,2′:1,6]pyrido[2,3-d]pyrimidin-2-yl)thiazol-2-amine (50 mg, 0.14 mmol) in dioxane/H₂O (10:1) (2 mL) was added N-(4-methyl-3-(4,4,5,5-tetramethyl-1,3,2-dioxaborolan-2-yl)phenyl)-4-(trifluoromethyl)picolinamide (58.2 mg, 0.14 mmol), Pd(dppf)Cl₂ (10 mg, 0.014 mmol), Cs₂CO₃ (137 mg, 0.42 mmol), the mixture was degassed three times and charged with N₂, stirred at 110° C. for 3 hrs. The reaction mixture was concentrated in vacuum and purified by column chromatography (DCM/MeOH=10/1) to afford the crude product, which was purified by Prep-HPLC (0.1% HCOOH) to afford N-(4-methyl-3-(2-(thiazol-2-ylamino)-8,9-dihydroimidazo[1′,2′:1,6]pyrido[2,3-d]pyrimidin-6-yl)phenyl)-4-(trifluoromethyl)picolinamide (3.8 mg, 3.5% yield) as a yellow solid.

¹H NMR (400 MHz, DMSO-d₆): δ 11.81 (s, 1H), 10.78 (s, 1H), 9.04 (d, J=4.8 Hz, 1H), 8.47 (s, 1H), 8.34 (s, 1H), 8.19 (s, 1H), 8.10 (d, J=4.8 Hz, 1H), 7.87 (s, 1H), 7.79 (d, J=8.0 Hz, 1H), 7.46 (d, J=3.6 Hz, 1H), 7.28-7.25 (m, 2H), 7.18 (d, J=3.6 Hz, 1H), 4.24 (t, J=9.2 Hz, 2H), 4.02 (t, J=9.2 Hz, 2H), 2.24 (s, 3H). LCMS (M+H⁺) m/z: 549.2.

### Example 256: Preparation of N-(4-methyl-3-(2-(thiazol-5-ylamino)-8,9-dihydroimidazo[1′,2′:1,6]pyrido[2,3-d]pyrimidin-6-yl)phenyl)-4-(trifluoromethyl)picolinamide (Compound 256)

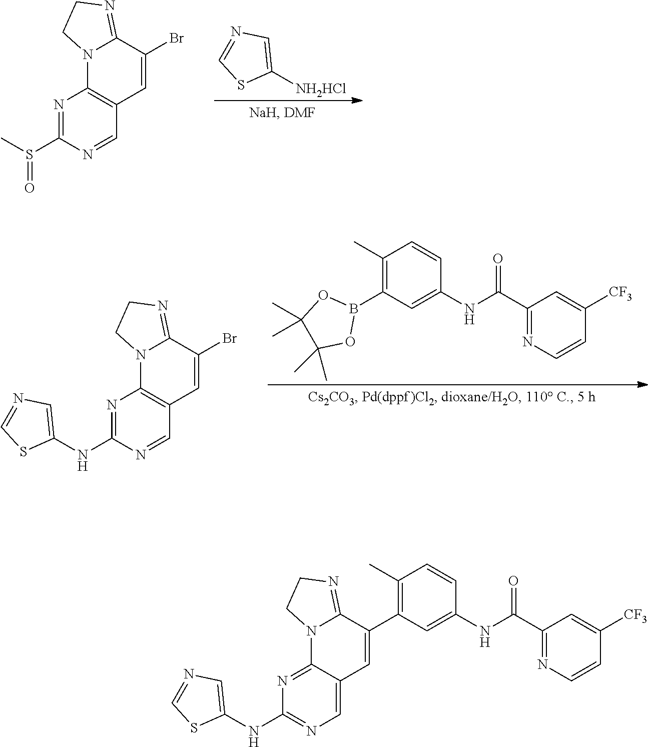

### Step 1: Synthesis of N-(6-bromo-8,9-dihydroimidazo[1′,2′:1,6]pyrido[2,3-d]pyrimidin-2-yl)thiazol-5-amine

To a solution of thiazol-5-amine hydrochloride (130 mg, 0.96 mmol) in DMF (2 mL) was added NaH (77 mg, 1.92 mmol) at 0° C. and the mixture was stirred at 0° C. for 1 h, 6-bromo-2-(methylsulfinyl)-8,9-dihydroimidazo[1′,2′:1,6]pyrido[2,3-d]pyrimidine (200 mg, 0.64 mmol) was added to the mixture. The resulting mixture was stirred for 2 h at r.t. H₂O (30 mL) was added, the reaction mixture was extracted with DCM (30 mL×3). The combined organic phases were washed with brine (60 mL), dried over Na2SO4, filtered and concentrated under vacuum to give the crude product, which was purified by column chromatography (DCM/MeOH=10/1) to afford the N-(6-bromo-8,9-dihydroimidazo[1′,2′:1,6]pyrido[2,3-d]pyrimidin-2-yl)thiazol-5-amine (70 mg, 31% yield) as a brown solid. LCMS (M+H⁺) m/z: 349.0 and 351.0.

### Step 2: Synthesis of N-(4-methyl-3-(2-(thiazol-5-ylamino)-8,9-dihydroimidazo[1′,2′:1,6]pyrido[2,3-d]pyrimidin-6-yl)phenyl)-4-(trifluoromethyl)picolinamide

To a mixture of N-(6-bromo-8,9-dihydroimidazo[1′,2′:1,6]pyrido[2,3-d]pyrimidin-2-yl)thiazol-5-amine (50 mg, 0.14 mmol) in dioxane/H₂O (10:1) (2 mL) was added N-(4-methyl-3-(4,4,5,5-tetramethyl-1,3,2-dioxaborolan-2-yl)phenyl)-4-(trifluoromethyl)picolinamide (70 mg, 0.17 mmol), Pd(dppf)Cl₂ (10 mg, 0.014 mmol), Cs₂CO₃ (137 mg, 0.42 mmol), the mixture was degassed three times and charged with N₂, stirred at 110° C. for 5 hrs. The reaction mixture was concentrated in vacuum and purified by column chromatography (DCM/MeOH=10/1) and by Prep-HPLC (0.1% HCOOH) to afford N-(4-methyl-3-(2-(thiazol-5-ylamino)-8,9-dihydroimidazo[1′,2′:1,6]pyrido[2,3-d]pyrimidin-6-yl)phenyl)-4-(trifluoromethyl)picolinamide (22.4 mg, 28.5% yield) as a yellow solid.

¹H NMR (400 MHz, DMSO-d₆): δ 11.11 (br s, 1H), 10.77 (s, 1H), 9.03 (d, J=5.2 Hz, 1H), 8.53 (s, 1H), 8.41 (s, 1H), 8.34 (s, 1H), 8.17 (s, 1H), 8.10-8.08 (m, 1H), 7.86 (d, J=2.0 Hz, 1H), 7.78 (dd, J=8.4, 2.0 Hz, 1H), 7.63 (s, 1H), 7.26-7.24 (m, 2H), 4.20-4.17 (m, 2H), 4.00 (t, J=8.8 Hz, 2H), 2.23 (s, 3H). LCMS (M+H⁺) m/z: 549.3.

### Example 257: Preparation of N-(3-(2-(isoxazol-5-ylamino)-8,9-dihydroimidazo[1′,2′:1,6]pyrido[2,3-d]pyrimidin-6-yl)-4-methylphenyl)-4-(trifluoromethyl)picolinamide (Compound 257)

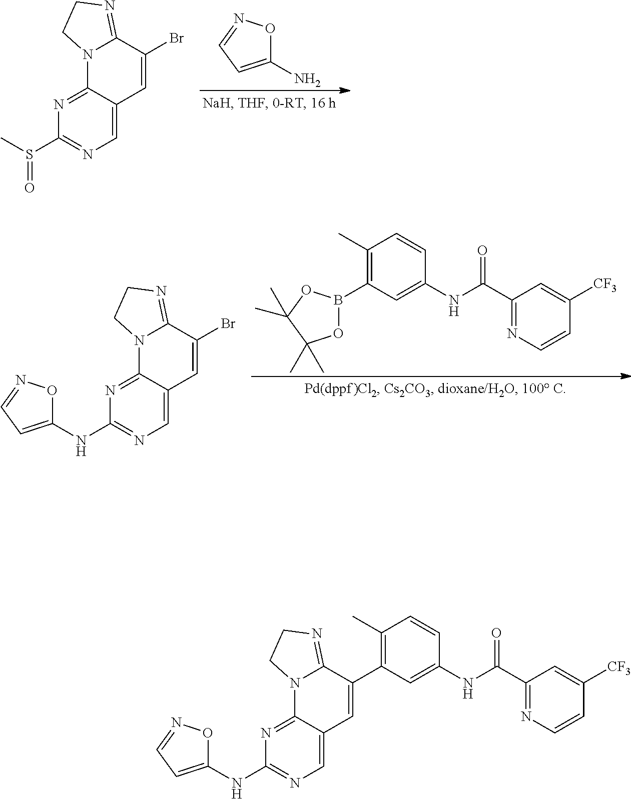

### Step 1: Synthesis of N-(6-bromo-8,9-dihydroimidazo[1′,2′:1,6]pyrido[2,3-d]pyrimidin-2-yl)isoxazol-5-amine

To a mixture of isoxazol-5-amine (150 mg, 1.79 mmol) in dry THF, was added NaH (71 mg, 1.79 mmol) at 0° C. The resulting mixture was stirred at r.t for 1.0 h under N2. Then 6-bromo-2-(methylsulfinyl)-8,9-dihydroimidazo[1′,2′:1,6]pyrido[2,3-d]pyrimidine (280 mg, 0.89 mmol) was added and the mixture was stirred at r.t for 16 h. The reaction mixture was quenched with cold-water (50 mL), extracted with DCM (50 mL×3). The organic phase was washed with brine, dried over Na2SO4, filtered and concentrated. The residue was and purified by silica column chromatography (DCM:MeOH=15:1) and then triturated with PE/EA (1:1, 2.0 mL) to afford N-(6-bromo-8,9-dihydroimidazo[1′,2′:1,6]pyrido[2,3-d]pyrimidin-2-yl)isoxazol-5-amine (116 mg, 38.9% yield) as a yellow solid. LCMS (M+H+) m/z: 333.1 and 335.1.

### Step 2: Synthesis of N-(3-(2-(isoxazol-5-ylamino)-8,9-dihydroimidazo[1′,2′:1,6]pyrido[2,3-d]pyrimidin-6-yl)-4-methylphenyl)-4-(trifluoromethyl)picolinamide

A mixture of 6-bromo-N-(oxetan-3-yl)-8,9-dihydroimidazo[1′,2′:1,6]pyrido[2,3-d]pyrimidin-2-amine (100 mg, 0.3 mmol), N-(4-methyl-3-(4,4,5,5-tetramethyl-1,3,2-dioxaborolan-2-yl)phenyl)-4-(trifluoromethyl)picolinamide (146 mg, 0.36 mmol), Cs₂CO₃ (196 mg, 0.6 mmol) and [1,1′-Bis(diphenylphosphino)ferrocene]dichloropalladium(II) (22 mg, 0.03 mmol) in dioxane (10.0 mL) and H₂O (1.1 mL) was stirred at 80° C. for 16 h under N₂. The reaction mixture was filtered and concentrated. The residue was purified by Prep-HPLC (0.1% F A) to afford N-(3-(2-(isoxazol-5-ylamino)-8,9-dihydroimidazo[1′,2′:1,6]pyrido[2,3-d]pyrimidin-6-yl)-4-methylphenyl)-4-(trifluoromethyl)picolinamide (5.1 mg, 3.2% yield) as a yellow solid. 1H NMR (400 MHz, CD₃OD): δ 9.12 (s, 1H), 8.97 (d, J=4.8 Hz, 1H), 8.43 (s, 1H), 8.38 (s, 1H), 8.25 (s, 1H), 7.95-7.94 (m, 2H), 7.80 (d, J=8.4 Hz, 1H), 7.47 (d, J=8.0 Hz, 1H), 6.62 (s, 1H), 4.85-4.80 (m, 2H), 4.23 (t, J=10.0 Hz, 2H), 2.30 (s, 3H). LCMS (M+H⁺) m/z: 533.2.

### Example 258: Preparation of N-(3-(2-(isoxazol-3-ylamino)-8,9-dihydroimidazo[1′,2′:1,6]pyrido[2,3-d]pyrimidin-6-yl)-4-methylphenyl)-4-(trifluoromethyl)picolinamide (Compound 258)

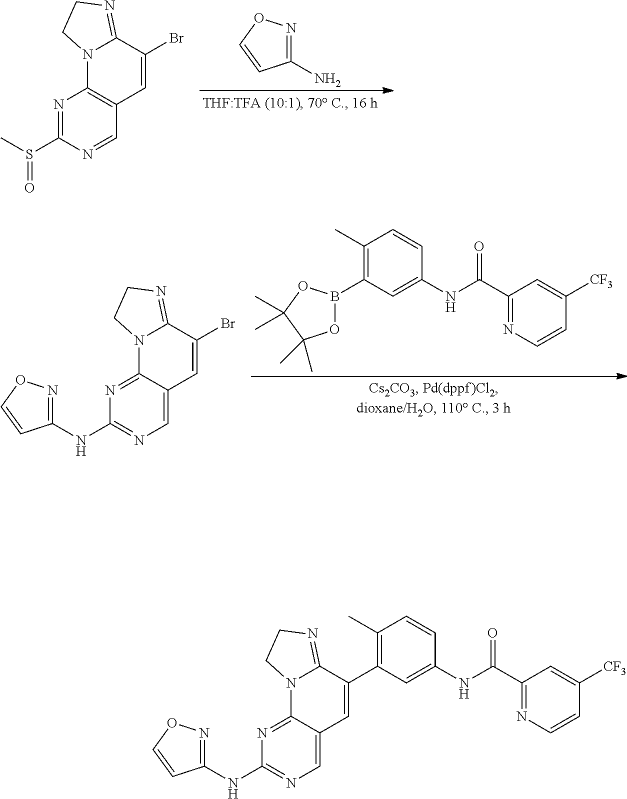

### Step 1: Synthesis of N-(6-bromo-8,9-dihydroimidazo[1′,2′:1,6]pyrido[2,3-d]pyrimidin-2-yl)isoxazol-3-amine

The solution of isoxazol-3-amine (134 mg, 1.6 mmol), 6-bromo-2-(methylsulfinyl)-8,9-dihydroimidazo[1′,2′:1,6]pyrido[2,3-d]pyrimidine (250 mg, 0.80 mmol) in THF:TFA (10:1) (2 mL) was stirred for 16 h at 70° C. The resulting mixture was concentrated and diluted with sat NaHCO₃ (30 mL), extracted with DCM (50 mL×3). The combined organic phases were washed with brine (60 mL), dried over Na₂SO₄, filtered and concentrated under vacuum to afford the crude product, which was purified by column chromatography (DCM/MeOH=10/1) to give N-(6-bromo-8,9-dihydroimidazo[1′,2′:1,6]pyrido[2,3-d]pyrimidin-2-yl)isoxazol-3-amine (130 mg, 48.9% yield) as a brown solid. LCMS (M+H⁺) m/z: 333.1 and 335.1.

### Step 2: Synthesis of N-(4-methyl-3-(2-(thiazol-2-ylamino)-8,9-dihydroimidazo[1′,2′:1,6]pyrido[2,3-d]pyrimidin-6-yl)phenyl)-4-(trifluoromethyl)picolinamide

To a mixture of N-(6-bromo-8,9-dihydroimidazo[1′,2′:1,6]pyrido[2,3-d]pyrimidin-2-yl)isoxazol-3-amine (50 mg, 0.15 mmol) in dioxane/H2O (10:1) (5 mL) was added N-(4-methyl-3-(4,4,5,5-tetramethyl-1,3,2-dioxaborolan-2-yl)phenyl)-4-(trifluoromethyl)picolinamide (61 mg, 0.15 mmol), Pd(dppf)Cl₂ (12 mg, 0.015 mmol), Cs₂CO₃ (147 mg, 0.45 mmol). The mixture was degassed three times and charged with N₂, stirred at 110° C. for 3 hrs. The reaction mixture was concentrated in vacuum and purified by column chromatography (DCM/MeOH=10/1) to afford the crude product, which was purified by Prep-HPLC (0.1% HCOOH) to afford N-(4-methyl-3-(2-(thiazol-2-ylamino)-8,9-dihydroimidazo[1′,2′:1,6]pyrido[2,3-d]pyrimidin-6-yl)phenyl)-4-(trifluoromethyl)picolinamide (3.6 mg, 3.2% yield) as a yellow solid. ¹H NMR (400 MHz, DMSO-d₆): δ 10.77 (s, 1H), 10.65 (s, 1H), 9.03 (d, J=4.8 Hz, 1H), 8.77 (d, J=1.6 Hz, 1H), 8.40 (s, 1H), 8.34 (s, 1H), 8.18 (s, 1H), 8.10-8.08 (m, 1H), 7.86 (d, J=2.0 Hz, 1H), 7.80-7.77 (m, 1H), 7.26-7.24 (m, 2H), 7.19 (d, J=1.2 Hz, 1H), 4.12 (t, J=9.2 Hz, 2H), 3.97 (t, J=9.2 Hz, 2H), 2.22 (s, 3H). LCMS (M+H⁺) m/z: 533.3.

### Example 259: Preparation of N-(4-methyl-3-(2-(oxazol-2-ylamino)-8,9-dihydroimidazo[1′,2′:1,6]pyrido[2,3-d]pyrimidin-6-yl)phenyl)-4-(trifluoromethyl)picolinamide (Compound 259)

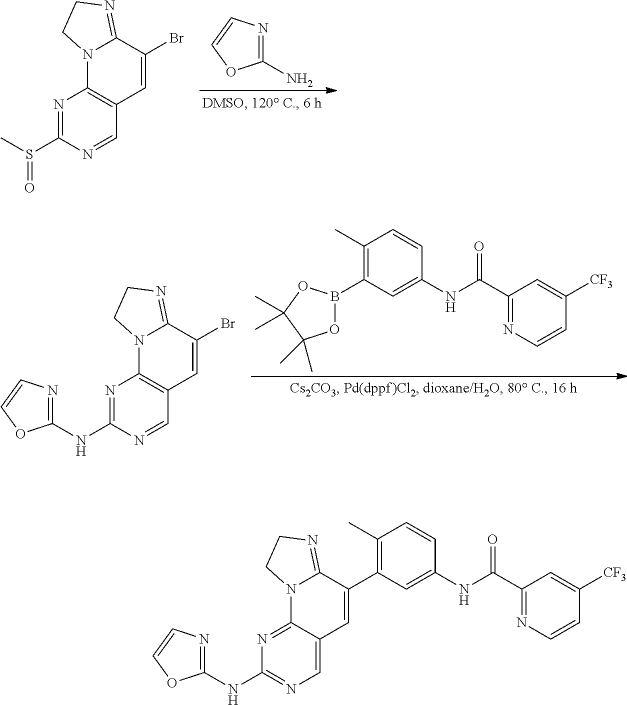

### Step 1: Synthesis of N-(6-bromo-8,9-dihydroimidazo[1′,2′:1,6]pyrido[2,3-d]pyrimidin-2-yl)oxazol-2-amine

A mixture of 6-bromo-2-(methylsulfinyl)-8,9-dihydroimidazo[1′,2′:1,6]pyrido[2,3-d]pyrimidine (150 mg, 0.48 mmol) and oxazol-2-amine (80 mg, 0.96 mmol) in DMSO (5 mL) was stirred at 120° C. for 16 h. The reaction mixture was diluted with water (20 mL) and extracted with DCM (20 mL×3), dried over Na2SO4, filtered. The combined organic layers were concentrated in vacuum and purified by column chromatography on silica gel (DCM/MeOH=15/1) to give crude product, which was purified by Prep-HPLC (0.1% NH₃—H₂O) to afford N-(6-bromo-8,9-dihydroimidazo[1′,2′:1,6]pyrido[2,3-d]pyrimidin-2-yl)oxazol-2-amine (40 mg, 25% yield) as brown solid. LCMS (M+H⁺) m/z: 333.0 and 335.0.

### Step 2: Synthesis of N-(4-methyl-3-(2-(oxazol-2-ylamino)-8,9-dihydroimidazo[1′,2′:1,6]pyrido[2,3-d]pyrimidin-6-yl)phenyl)-4-(trifluoromethyl)picolinamide

A mixture of N-(6-bromo-8,9-dihydroimidazo[1′,2′:1,6]pyrido[2,3-d]pyrimidin-2-yl)oxazol-2-amine (40 mg, 0.12 mmol), N-(4-methyl-3-(4,4,5,5-tetramethyl-1,3,2-dioxaborolan-2-yl)phenyl)-4-(trifluoromethyl)picolinamide (48 mg, 0.28 mmol), Cs₂CO₃ (118 mg, 0.36 mmol) and Pd(dppf)Cl₂ (8 mg, 0.01 mmol) in dioxane (5 mL) and water (0.5 mL) was degassed, charged with N₂ three times and stirred at 80° C. for 16 h. The reaction mixture was concentrated in vacuum and purified by column chromatography on silica gel (DCM/MeOH=10/1) to give crude product, which was purified by Prep-HPLC (0.1% FA) to afford N-(4-methyl-3-(2-(oxazol-2-ylamino)-8,9-dihydroimidazo[1′,2′:1,6]pyrido[2,3-d]pyrimidin-6-yl)phenyl)-4-(trifluoromethyl)picolinamide (3.7 mg, 5.8% yield) as a yellow solid.

¹H NMR (400 MHz, DMSO-d₆): δ 10.79 (s, 1H), 10.28 (s, 1H), 9.03 (d, J=5.2 Hz, 1H), 8.55 (s, 1H), 8.34 (s, 1H), 8.09 (d, J=4.4 Hz, 1H), 7.88 (s, 1H), 7.80 (d, J=7.2 Hz, 1H), 7.34 (s, 1H), 7.26 (d, J=8.4 Hz, 1H), 7.11 (d, J=2.4 Hz, 1H), 6.61 (s, 1H), 4.12 (t, J=8.4 Hz, 2H), 4.03 (t, J=8.4 Hz, 2H), 2.23 (s, 3H). LCMS (M+H⁺) m/z: 533.2.

### Example 260: Preparation of N-(5-chloro-4-(trifluoromethyl)pyridin-2-yl)-4-methyl-3-(2-((1-methyl-1H-pyrazol-4-yl)amino)-8,9-dihydroimidazo[1′,2′:1,6]pyrido[2,3-d]pyrimidin-6-yl)benzamide (Compound 260)

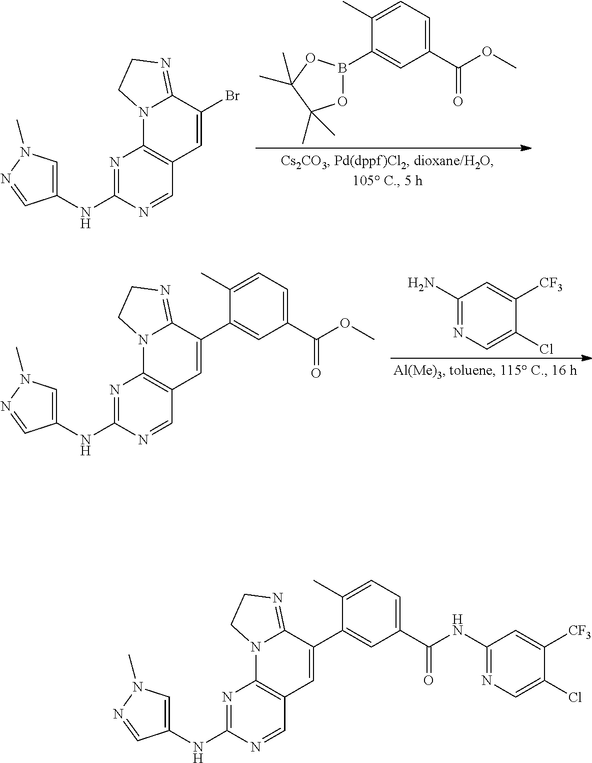

### Step 1: Synthesis of methyl 4-methyl-3-(2-((1-methyl-1H-pyrazol-4-yl)amino)-8,9-dihydroimidazo[1′,2′:1,6]pyrido[2,3-d]pyrimidin-6-yl)benzoate

To a mixture of 6-bromo-N-(1-methyl-1H-pyrazol-4-yl)-8,9-dihydroimidazo[1′,2′:1,6]pyrido[2,3-d]pyrimidin-2-amine (400 mg, 1.16 mmol), methyl 4-methyl-3-(4,4,5,5-tetramethyl-1,3,2-dioxaborolan-2-yl)benzoate (319 mg, 1.16 mmol) in dioxane/H₂O (20 mL/2 mL) was added Pd(dppf)Cl₂ (85 mg, 0.116 mmol), Cs₂CO₃ (1.13 g, 3.47 mmol). The mixture was stirred for 5 h at 105° C. under N₂. The reaction mixture was concentrated in vacuum and purified by column chromatography on silica gel (DCM/MeOH=15/1) to afford methyl 4-methyl-3-(2-((1-methyl-1H-pyrazol-4-yl)amino)-8,9-dihydroimidazo[1′,2′:1,6]pyrido[2,3-d]pyrimidin-6-yl)benzoate (350 mg, 73.1% yield) as white solid. LCMS (M+H)⁺ m/z: 416.1.

### Step 2: Synthesis of N-(5-chloro-4-(trifluoromethyl)pyridin-2-yl)-4-methyl-3-(2-((1-methyl-1H-pyrazol-4-yl)amino)-8,9-dihydroimidazo[1′,2′:1,6]pyrido[2,3-d]pyrimidin-6-yl)benzamide formic acid salt

To a mixture of methyl 4-methyl-3-(2-((1-methyl-1H-pyrazol-4-yl)amino)-8,9-dihydroimidazo[1′,2′:1,6]pyrido[2,3-d]pyrimidin-6-yl)benzoate (90 mg, 0.217 mmol), 5-chloro-4-(trifluoromethyl)pyridin-2-amine (80 mg, 0.325 mmol) in dry toluene (10 mL) was added Al(Me)₃ (0.33 mL, 2M, 0.65 mmol) at r.t under N₂. The mixture was stirred at 115° C. for 16 h. The reaction mixture was concentrated in vacuum and purified by column chromatography on silica gel (DCM/MeOH=10/1) to give crude product, which was purified by Prep-HPLC (0.1% NH₃—H₂O) to afford N-(5-chloro-4-(trifluoromethyl)pyridin-2-yl)-4-methyl-3-(2-((1-methyl-1H-pyrazol-4-yl)amino)-8,9-dihydroimidazo[1′,2′:1,6]pyrido[2,3-d]pyrimidin-6-yl)benzamide formic acid salt (25.0 mg, 18.4% yield) as yellow solid.

¹H NMR (400 MHz, DMSO-d₆): δ 11.40 (s, 1H), 9.80-9.68 (m, 1H), 8.75 (s, 1H), 8.68 (s, 1H), 8.20 (s, 1H), 7.96-7.93 (m, 3H), 7.56 (s, 1H), 7.40 (d, J=8.8 Hz, 1H), 7.20 (s, 1H), 4.24-4.08 (m, 2H), 3.97 (t, J=9.2 Hz, 2H), 3.83 (s, 3H), 2.33 (s, 3H). LCMS (M+H⁺) m/z: 580.3.

### Example 261: Preparation of N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-2-(trifluoromethyl)isonicotinamide (Compound 261)

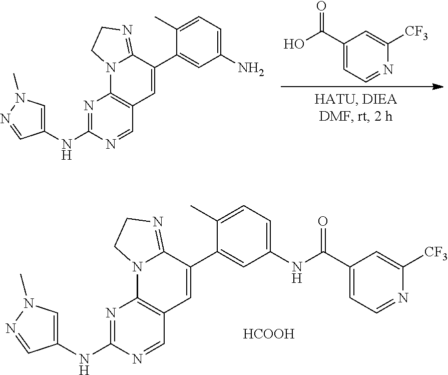 [SMILES: Cc1ccc(N)cc1C1=Cc2cnc(Nc3cnn(C)c3)nc2N2CCN=C12 ; O=C(O)c1ccnc(C(F)(F)F)c1 ; Cc1ccc(NC(=O)c2ccnc(C(F)(F)F)c2)cc1C1=Cc2cnc(Nc3cnn(C)c3)nc2N2CCN=C12 ; "HCOOH"]

The preparation of 6-(5-Amino-2-methylphenyl)-N-(1-methyl-1H-pyrazol-4-yl)-8,9-dihydroimidazo[1′,2′:1,6]pyrido[2,3-d]pyrimidin-2-amine was described in Example 196

### Step 1: Synthesis of N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-2-(trifluoromethyl)isonicotinamide formate

HATU (92 mg, 0.24 mmol) was added to the mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (60 mg, 0.16 mmol), 2-(trifluoromethyl)isonicotinic acid (37 mg, 0.19 mmol), DIEA (62 mg, 0.48 mmol) in DMF (1 mL). The mixture was stirred at rt for 2 hours, diluted with water (50 mL), filtered. The solid obtained was dried and purified by column chromatography on silica gel (DCM:MeOH=10:1) to give the crude product, which was further purified by Prep-HPLC (0.1% HCOOH) to afford N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-2-(trifluoromethyl)isonicotinamide formate (50 mg, 56.9% yield) as a yellow solid.

¹H NMR (400 MHz, DMSO-d₆): δ 10.67 (s, 1H), 9.83 (br, 1H), 8.99 (d, J=5.2 Hz, 1H), 8.37 (s, 1H), 8.33 (s, 1H), 8.20 (d, J=4.8 Hz, 1H), 8.17 (s, 1H), 7.92 (s, 1H), 7.70-7.67 (m, 2H), 7.56 (s, 1H), 7.27 (d, J=8.0 Hz, 1H), 7.22 (s, 1H), 4.24-4.20 (m, 2H), 3.97 (t, J=9.2 Hz, 2H), 3.58 (s, 3H), 2.23 (s, 3H). LCMS (M+H⁺) m/z: 546.6.

### Example 262: Preparation of 3-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (Compound 262)

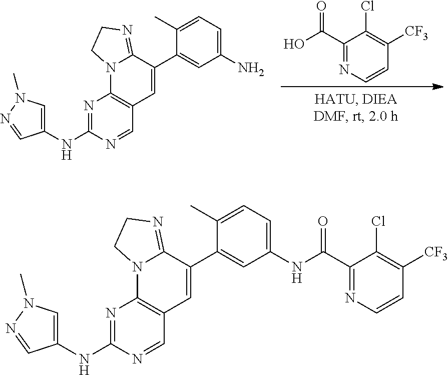 [SMILES: Cc1ccc(N)cc1C1=Cc2cnc(Nc3cnn(C)c3)nc2N2CCN=C12 ; O=C(O)c1nccc(C(F)(F)F)c1Cl ; Cc1ccc(NC(=O)c2nccc(C(F)(F)F)c2Cl)cc1C1=Cc2cnc(Nc3cnn(C)c3)nc2N2CCN=C12]

### Step 1: Synthesis of 3-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide

A mixture of dihydroimidazo[1′,2′:1,6]pyrido[2,3-d]pyrimidin-2-amine (60 mg, 0.16 mmol), 3-chloro-4-(trifluoromethyl)picolinic acid (36 mg, 0.16 mmol), HATU (92 mg, 0.24 mmol) and DIEA (62 mg, 0.48 mmol) in dry DMF (3 mL) was stirred at r.t for 2 h. The reaction mixture was diluted with H₂O (20 mL) and extracted with DCM (30 mL×3). The combined organic layers were dried over Na₂SO₄, concentrated in vacuum and purified by column chromatography on silica gel (DCM/MeOH=10/1) to give crude product, which was triturated with MeOH (1 mL) to afford 3-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (27.4 mg, 29.3% yield) as yellow solid.

¹H NMR (400 MHz, DMSO-d₆): δ 10.78 (s, 1H), 8.78-9.69 (m, 1H), 8.88 (d, J=5.2 Hz, 1H), 8.33 (s, 1H), 8.05 (d, J=4.8 Hz, 1H), 7.93 (s, 1H), 7.65 (s, 1H), 7.55 (dd, J=8.4, 2.4 Hz, 2H), 7.28-7.21 (m, 2H), 4.21-4.20 (m, 2H), 3.97 (t, J=8.2 Hz, 2H), 3.82 (s, 3H), 2.21 (s, 3H). LCMS (M+H⁺) m/z: 580.6.

### Example 263: Preparation of 5-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (Compound 263)

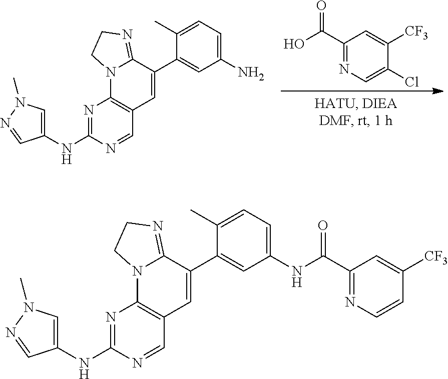 [SMILES: Cc1ccc(N)cc1C1=Cc2cnc(Nc3cnn(C)c3)nc2N2CCN=C12 ; O=C(O)c1cc(C(F)(F)F)c(Cl)cn1 ; Cc1ccc(NC(=O)c2cc(C(F)(F)F)ccn2)cc1C1=Cc2cnc(Nc3cnn(C)c3)nc2N2CCN=C12]

### Step 1: Synthesis of 5-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide

To a solution of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (60 mg, 0.16 mmol) in DMF (2 mL) was added 5-chloro-4-(trifluoromethyl)picolinic acid (36 g, 0.16 mmol), HATU (92 mg, 0.24 mmol), DIPEA (62 mg, 0.48 mmol). The reaction mixture was stirred for 1 h at RT. Water (10 mL) was added to the reaction mixture, the resulting solid was filtered and purified by column chromatography (DCM:MeOH=15:1) to afford 5-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (34.5 mg, 36.9% yield) as a yellow solid.

¹H NMR (400 MHz, DMSO-d₆): δ 10.79 (s, 1H), 9.87 (s, 1H), 9.09 (s, 1H), 8.37 (s, 2H), 7.93 (s, 1H), 7.84 (d, J=2.0 Hz, 1H), 7.78 (dd, J=8.4, 2.0 Hz, 1H), 7.57 (s, 1H), 7.25 (d, J=8.4 Hz, 1H), 4.25-4.21 (m, 2H), 3.98 (t, J=8.4 Hz, 2H), 3.83 (s, 3H), 2.22 (s, 3H). LCMS (M+H⁺) m/z: 580.2.

### Example 264: Preparation of 4-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-3-(trifluoromethyl)benzamide (Compound 264)

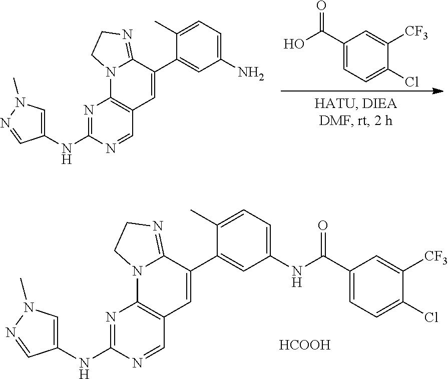 [SMILES: Cc1ccc(N)cc1C1=Cc2cnc(Nc3cnn(C)c3)nc2N2CCN=C12 ; O=C(O)c1ccc(Cl)c(C(F)(F)F)c1 ; Cc1ccc(NC(=O)c2ccc(Cl)c(C(F)(F)F)c2)cc1C1=Cc2cnc(Nc3cnn(C)c3)nc2N2CCN=C12 ; "HCOOH"]

### Step 1: Synthesis of 4-Chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-3-(trifluoromethyl)benzamide formate

HATU (92 mg, 0.24 mmol) was added to the mixture of 6-(5-amino-2-methylphenyl)-N-(1-methyl-1H-pyrazol-4-yl)-8,9-dihydroimidazo[1′,2′:1,6]pyrido[2,3-d]pyrimidin-2-amine (60 mg, 0.16 mmol), 4-chloro-3-(trifluoromethyl)benzoic acid (43 mg, 0.19 mmol), DIEA (62 mg, 0.48 mmol) in DMF (1 mL). The mixture was stirred at r.t for 2 hours. The reaction mixture was diluted with water (50 mL), The solid obtained was filtered, dried and purified by column chromatography on silica gel (DCM:MeOH=10:1) and Prep-HPLC (0.1% HCOOH) to afford 4-chloro-N-(4-methyl-3-(2-((1-methyl-1H-pyrazol-4-yl)amino)-8,9-dihydroimidazo[1′,2′:1,6]pyrido[2,3-d]pyrimidin-6-yl)phenyl)-3-(trifluoromethyl)benzamide formate (45 mg, 48.2% yield) as a yellow solid.

¹H NMR (400 MHz, DMSO-d₆): δ 10.48 (s, 1H), 9.80 (br, 1H), 8.39 (s, 1H), 8.32 (s, 1H), 8.26 (d, J=8.8 Hz, 1H), 8.19 (s, 1H), 7.93-7.91 (m, 2H), 7.68-7.65 (m, 2H), 7.56-7.54 (m, 1H), 7.24 (d, J=8.0 Hz, 1H), 7.19 (s, 1H), 4.20-4.16 (m, 2H), 3.97 (t, J=9.2 Hz, 2H), 3.82 (s, 3H), 2.22 (s, 3H). LCMS (M+H⁺) m/z: 579.2.

### Example 265: Preparation of N-(4-methyl-3-(2-((1-methyl-1H-pyrazol-4-yl)amino)-9,10-dihydro-8H-pyrido[1,6-a:2,3-d′]dipyrimidin-6-yl)phenyl)-4-(trifluoromethyl)picolinamide formate (Compound 265)

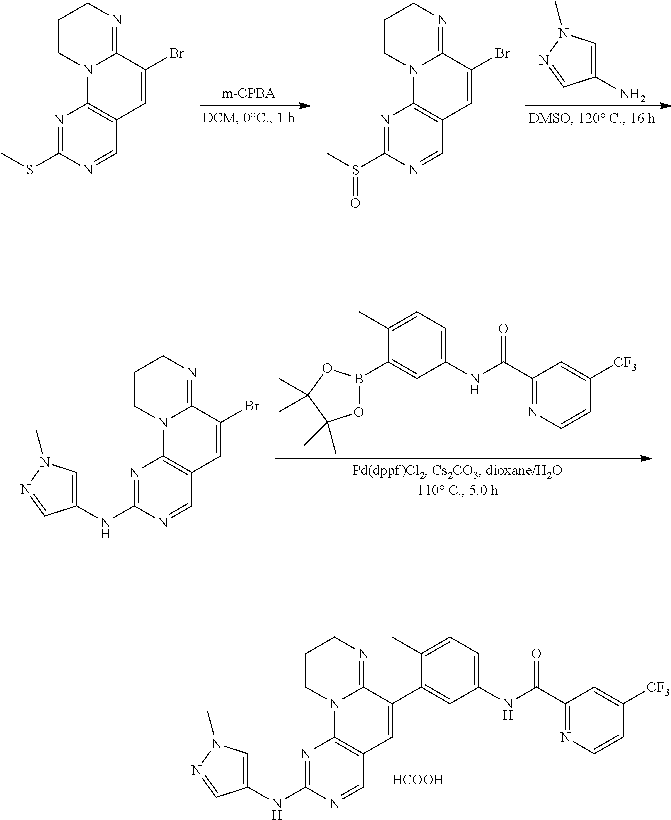

### Step 1: Synthesis of 6-(2,4-Difluorophenoxy)-2-(methylsulfonyl)-8,9-dihydroimidazo[1′,2′:1,6]pyrido[2,3-d]pyrimidine

To a mixture of 6-bromo-2-(methylthio)-9,10-dihydro-8H-pyrido[1,6-a:2,3-d′]dipyrimidine (300 mg, 0.96 mmol) in DCM (10 mL), was added m-CPBA (390 mg, 1.93 mmol) at 0° C. The reaction mixture was stirred at 0° C. for 1 hours. The mixture was concentrated to give the crude product, which was purified on silica column chromatography (DCM:MeOH=10:1) to afford 6-(2,4-difluorophenoxy)-2-(methylsulfonyl)-8,9-dihydroimidazo [1′,2′:1,6]pyrido[2,3-d]pyrimidine (300 mg, crude) as a brown solid. LCMS (M+H⁺) m/z: 327.0 and 329.0

### Step 2: Synthesis of 6-Bromo-N-(1-methyl-1H-pyrazol-4-yl)-9,10-dihydro-8H-pyrido[1,6-a:2,3-d′]dipyrimidin-2-amine

To a solution of 6-bromo-2-(methylsulfinyl)-9,10-dihydro-8H-pyrido[1,6-a:2,3-d′]dipyrimidine (300 mg, 0.92 mmol) in DMSO (10 mL), was added 1-methyl-1H-pyrazol-4-amine hydrochloride (122 mg, 0.92 mmol). The reaction mixture was stirred at 120° C. for 16 hours. The mixture was purified by Prep-HPLC (0.1% ammonia) to give 6-bromo-N-(1-methyl-1H-pyrazol-4-yl)-9,10-dihydro-8H-pyrido[1,6-a:2,3-d′]dipyrimidin-2-amine (90 mg, 27% yield) as a brown solid. LCMS (M+H⁺) m/z: 359.9 and 361.9

### Step 3: Synthesis of N-(4-methyl-3-(2-((1-methyl-1H-pyrazol-4-yl)amino)-9,10-dihydro-8H-pyrido[1,6-a:2,3-d′]dipyrimidin-6-yl)phenyl)-4-(trifluoromethyl)picolinamide formate

A mixture of 6-bromo-N-(1-methyl-1H-pyrazol-4-yl)-9,10-dihydro-8H-pyrido[1,6-a:2,3-d′]dipyrimidin-2-amine (70 mg, 1.17 mmol), N-(4-methyl-3-(4,4,5,5-tetramethyl-1,3,2-dioxaborolan-2-yl)phenyl)-4-(trifluoromethyl)picolinamide (95 mg, 0.23 mmol), Cs₂CO₃ (190 mg, 0.58 mmol) and Pd(dppf)Cl₂ (87.8 mg, 0.12 mmol) in dioxane/H₂O (10:1) (2 mL) under N₂ was stirred at 110° C. for 5 hours. The reaction mixture was concentrated and purified by silica column (DCM:MeOH=10:1) to afford the crude product, which was further purified by Prep-HPLC (0.1% FA) to give N-(4-methyl-3-(2-((1-methyl-1H-pyrazol-4-yl)amino)-9,10-dihydro-8H-pyrido[1,6-a:2,3-d′]dipyrimidin-6-yl)phenyl)-4-(trifluoromethyl)picolinamide formate (39.7 mg, 36.5% yield) as a yellow solid.

¹H NMR (400 MHz, DMSO-d₆): δ 10.75 (s, 1H), 9.93 (s, 1H), 9.03 (d, J=4.8 Hz, 1H), 8.45 (s, 1H), 8.33 (s, 1H), 8.24 (s, 1H), 8.09 (d, J=4.8 Hz, 1H), 7.89 (s, 1H), 7.78-7.76 (m, 2H), 7.60 (s, 1H), 7.23 (d, J=7.6 Hz, 1H), 4.23 (t, J=7.6 Hz, 2H), 3.84 (s, 3H), 3.42-3.36 (m, 2H), 2.32 (s, 3H), 1.99-1.97 (m, 2H). LCMS (M+H⁺) m/z: 560.3.

### Example 266: Preparation of 3-(4-fluorophenyl)-1-isopropyl-N-(4-methyl-3-(2-((6-methylpyridin-3-yl)amino)-8,9-dihydroimidazo[1′,2′:1,6]pyrido[2,3-d]pyrimidin-6-yl)phenyl)-2,4-dioxo-1,2,3,4-tetrahydropyrimidine-5-carboxamide (Compound 266)

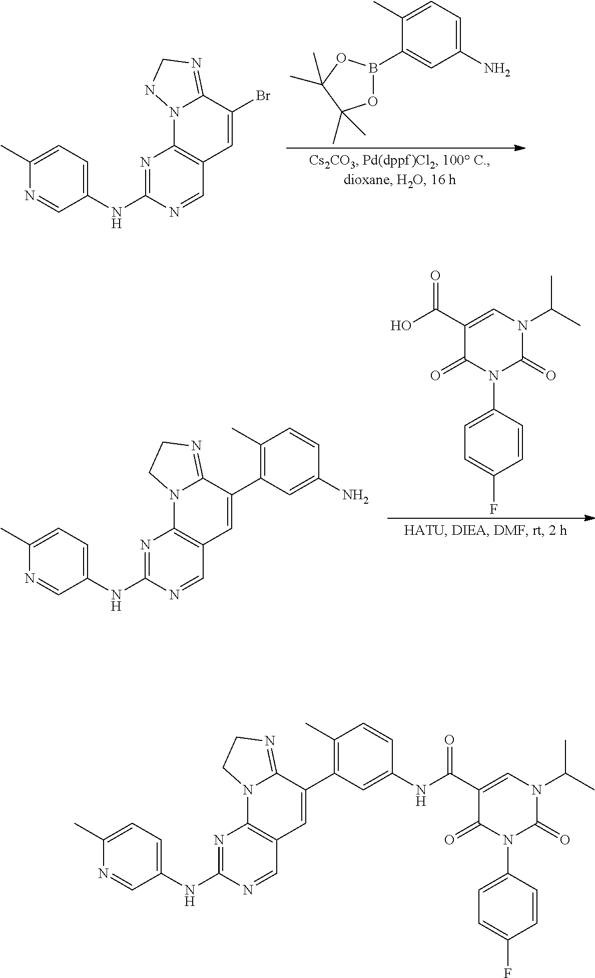

The preparation of 6-bromo-N-(6-methylpyridin-3-yl)-8,9-dihydroimidazo[1′,2′:1,6]pyrido[2,3-d]pyrimidin-2-amine was described in Example 208

### Step 1: Synthesis of 6-(5-amino-2-methylphenyl)-N-(6-methylpyridin-3-yl)-8,9-dihydroimidazo[1′,2′:1,6]pyrido[2,3-d]pyrimidin-2-amine

A mixture of 6-bromo-N-(6-methylpyridin-3-yl)-8,9-dihydroimidazo[1′,2′:1,6]pyrido[2,3-d]pyrimidin-2-amine (40 mg, 0.112 mmol), 4-methyl-3-(4,4,5,5-tetramethyl-1,3,2-dioxaborolan-2-yl)aniline (55 mg, 0.134 mmol), Cs₂CO₃ (110 mg, 0.337 mmol) and Pd(dppf)Cl₂ (8 mg, 0.01 mmol) in dioxane (5 mL) and water (0.5 mL) was degassed, charged with N₂ three times and stirred at 100° C. for 16 h. The reaction mixture was concentrated in vacuum and purified by column chromatography on silica gel (DCM/MeOH=10/1) to afford 6-(5-amino-2-methylphenyl)-N-(6-methylpyridin-3-yl)-8,9-dihydroimidazo[1′,2′:1,6]pyrido[2,3-d]pyrimidin-2-amine (30 mg, 70% yield) as brown solid. LCMS (M+H⁺) m/z: 384.3.

### Step 2: Synthesis of 3-(4-fluorophenyl)-1-isopropyl-N-(4-methyl-3-(2-((6-methylpyridin-3-yl)amino)-8,9-dihydroimidazo[1′,2′:1,6]pyrido[2,3-d]pyrimidin-6-yl)phenyl)-2,4-dioxo-1,2,3,4-tetrahydropyrimidine-5-carboxamide formic acid

To a solution of 6-(5-amino-2-methylphenyl)-N-(6-methylpyridin-3-yl)-8,9-dihydroimidazo[1′,2′:1,6]pyrido[2,3-d]pyrimidin-2-amine (20 mg, 0.052 mmol), 3-(4-fluorophenyl)-1-isopropyl-2,4-dioxo-1,2,3,4-tetrahydropyrimidine-5-carboxylic acid (17 mg, 0.057 mmol) and HATU (40 mg, 0.104 mmol) in DMF (3.0 mL) was added DIEA (14 mg, 0.104 mmol). The resulting mixture was stirred at r.t. for 16 h under N₂. The reaction mixture was quenched with water (20 mL) and extracted with DCM (20 mL×3). The combined organic layers were washed with brine (20 mL), dried over Na₂SO₄, filtered, concentrated to afford crude product, which was purified by Prep-HPLC (0.1% FA) to afford 3-(4-fluorophenyl)-1-isopropyl-N-(4-methyl-3-(2-((6-methylpyridin-3-yl)amino)-8,9-dihydroimidazo[1′,2′:1,6]pyrido[2,3-d]pyrimidin-6-yl)phenyl)-2,4-dioxo-1,2,3,4-tetrahydropyrimidine-5-carboxamide formic acid (9.3 mg, 25.3% yield) as a yellow solid.

¹H NMR (400 MHz, DMSO-d₆): δ 10.82 (s, 1H), 9.89 (s, 1H), 8.83 (s, 1H), 8.62 (s, 1H), 8.36 (s, 1H), 8.15 (s, 1H), 8.12-8.10 (m, 1H), 7.57-7.55 (m, 2H), 7.43-7.40 (m, 2H), 7.34 (t, J=8.8 Hz, 2H), 7.22-7.17 (m, 3H), 4.80-4.73 (m, 1H), 4.12 (t, J=9.6 Hz, 2H), 3.95 (t, J=9.6 Hz, 2H), 2.41 (s, 3H), 2.20 (s, 3H), 1.40 (d, J=6.8 Hz, 6H). LCMS (M+H⁺) m/z: 658.3.

### Example 267: Preparation of N-(2-fluoro-4-methyl-5-(2-((6-methylpyridin-3-yl)amino)-8,9-dihydroimidazo[1′,2′:1,6]pyrido[2,3-d]pyrimidin-6-yl)phenyl)-4-(trifluoromethyl)picolinamide (Compound 267)

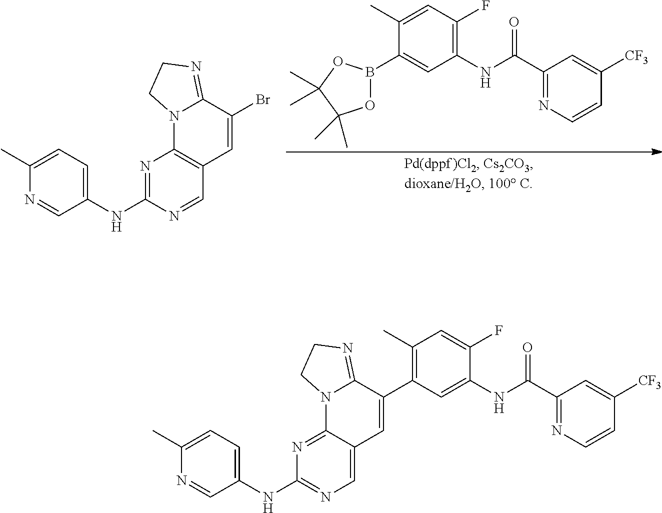

### Step 1: Synthesis of N-(2-fluoro-4-methyl-5-(2-((6-methylpyridin-3-yl)amino)-8,9-dihydroimidazo[1′,2′:1,6]pyrido[2,3-d]pyrimidin-6-yl)phenyl)-4-(trifluoromethyl)picolinamide

To a solution of 6-bromo-N-(6-methylpyridin-3-yl)-8,9-dihydroimidazo[1′,2′:1,6]pyrido[2,3-d]pyrimidin-2-amine (110 mg, 0.31 mmol) in dioxane/H₂O (10 mL/1 mL) was added N-(2-fluoro-4-methyl-5-(4,4,5,5-tetramethyl-1,3,2-dioxaborolan-2-yl)phenyl)-4-(trifluoromethyl)picolinamide (156 mg, 0.37 mmol), Pd(dppf)Cl₂ (23 mg, 0.031 mmol), Cs₂CO₃ (301 mg, 0.92 mmol). The mixture was stirred for 2 h at 120° C. under N₂ and concentrated. The residue was purified by column chromatography (EA:MeOH=10:1) to afford N-(2-fluoro-4-methyl-5-(2-((6-methylpyridin-3-yl)amino)-8,9-dihydroimidazo[1′,2′:1,6]pyrido[2,3-d]pyrimidin-6-yl)phenyl)-4-(trifluoromethyl)picolinamide (30 mg, 20.5% yield) as a yellow solid.

¹H NMR (400 MHz, DMSO-d₆): δ 10.49 (s, 1H), 10.02 (br s, 1H), 9.06 (d, J=5.2 Hz, 1H), 8.85 (d, J=2.0 Hz, 1H), 8.50 (s, 1H), 8.33 (s, 1H), 8.14-8.10 (m, 2H), 7.88 (d, J=8.0 Hz, 1H), 7.41 (s, 1H), 7.32 (d, J=11.6 Hz, 1H), 7.22 (d, J=8.4 Hz, 1H), 4.24-4.20 (m, 2H), 4.01-3.96 (m, 2H), 2.42 (s, 3H), 2.23 (s, 3H). LCMS (M+H⁺) m/z: 575.2.

### Example 268: Preparation of N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (Compound 268)

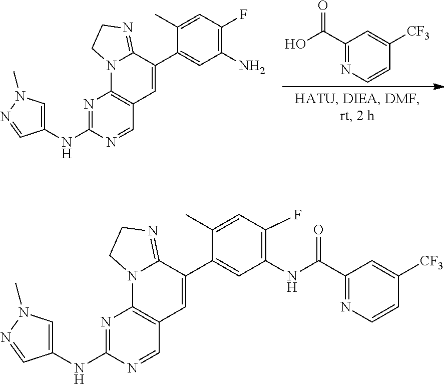 [SMILES: Cc1cc(F)c(N)cc1C1=Cc2cnc(Nc3cnn(C)c3)nc2N2CCN=C12 ; O=C(O)c1cc(C(F)(F)F)ccn1 ; Cc1cc(F)c(NC(=O)c2cc(C(F)(F)F)ccn2)cc1C1=Cc2cnc(Nc3cnn(C)c3)nc2N2CCN=C12]

The preparation of 6-(5-amino-4-fluoro-2-methylphenyl)-N-(1-methyl-1H-pyrazol-4-yl)-8,9-dihydroimidazo[1′,2′:1,6]pyrido[2,3-d]pyrimidin-2-amine was described in Example 201

### Step 1: Synthesis of N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide

A mixture of 6-(5-amino-4-fluoro-2-methylphenyl)-N-(1-methyl-1H-pyrazol-4-yl)-8,9-dihydroimidazo[1′,2′:1,6]pyrido[2,3-d]pyrimidin-2-amine (150 mg, 0.38 mmol), 4-(trifluoromethyl)picolinic acid (74 mg, 0.38 mmol), HATU (219 mg, 0.58 mmol) and DIEA (149 mg, 1.15 mmol) in DMF (5 mL) was stirred at RT for 2 h. The reaction mixture was diluted with water (20 mL), extracted with EA (50 mL×3). The combined organic phase was washed with brine (50 mL×3), dried with Na₂SO₄, filtered and concentrated. The residue was purified by Prep-HPLC (0.1% FA) to afford N-(2-fluoro-4-methyl-5-(2-((1-methyl-1H-pyrazol-4-yl)amino)-8,9-dihydroimidazo[1′,2′:1,6]pyrido[2,3-d]pyrimidin-6-yl)phenyl)-4-(trifluoromethyl)picolinamide (10.3 mg, 4.8% yield) as a yellow solid.

¹H NMR (400 MHz, DMSO-d₆): δ 10.46 (s, 1H), 9.80 (br, 1H), 9.05 (d, J=4.8 Hz, 1H), 8.33-8.30 (s, 4H), 8.13 (d, J=4.0 Hz, 1H), 7.92 (s, 1H), 7.85 (d, J=8.4 Hz, 1H), 7.53-7.55 (m, 1H), 7.27 (d, J=11.6 Hz, 1H), 7.20 (s, 1H), 4.20-4.16 (m, 2H), 3.99-3.94 (m, 2H), 3.82 (s, 3H), 2.25 (s, 3H). ¹⁹F NMR (400 MHz, DMSO-d₆): δ-126.94 (s, 1F), −63.47 (s, 3F). LCMS (M+H⁺) m/z: 564.1.

### Example 269: Preparation of N-(2-fluoro-4-methyl-5-(2-((1-methyl-1H-pyrazol-3-yl)amino)-8,9-dihydroimidazo[1′,2′:1,6]pyrido[2,3-d]pyrimidin-6-yl)phenyl)-4-(trifluoromethyl)picolinamide (Compound 269)

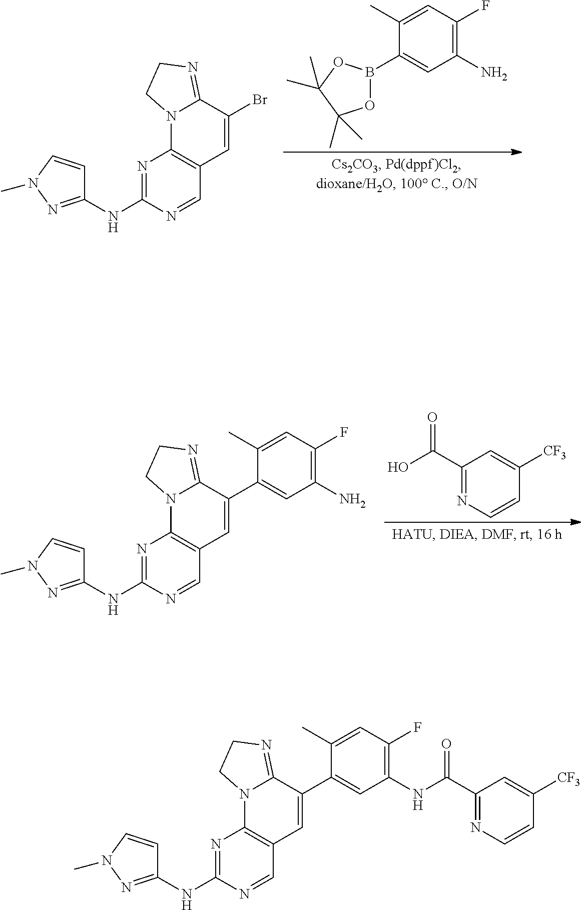

The preparation of 6-bromo-N-(1-methyl-1H-pyrazol-3-yl)-8,9-dihydroimidazo[1′,2′:1,6]pyrido[2,3-d]pyrimidin-2-amine was described in Example 234

### Step 1: Synthesis of 6-(5-amino-4-fluoro-2-methylphenyl)-N-(1-methyl-1H-pyrazol-3-yl)-8,9-dihydroimidazo[1′,2′:1,6]pyrido[2,3-d]pyrimidin-2-amine

A mixture of 6-bromo-N-(1-methyl-1H-pyrazol-3-yl)-8,9-dihydroimidazo[1′,2′:1,6]pyrido[2,3-d]pyrimidin-2-amine (180 mg, 0.52 mmol), 2-fluoro-4-methyl-5-(4,4,5,5-tetramethyl-1,3,2-dioxaborolan-2-yl)aniline (144 mg, 0.57 mmol), Cs₂CO₃ (508 mg, 1.56 mmol) and [1,1′-Bis(diphenylphosphino)ferrocene]dichloropalladium(II) (38 mg, 0.052 mmol) in dioxane (5.0 mL) and H₂O (0.5 mL) was stirred at 100° C. for 16 h under N₂. The reaction mixture was concentrated and purified by column chromatography on silica gel (DCM:MeOH=10:1) to afford 6-(5-amino-4-fluoro-2-methylphenyl)-N-(1-methyl-1H-pyrazol-3-yl)-8,9-dihydroimidazo[1′,2′:1,6]pyrido[2,3-d]pyrimidin-2-amine (100 mg, 49% yield) as a yellow solid. LCMS (M+H⁺) m/z: 391.0.

### Step 2: Synthesis of N-(2-fluoro-4-methyl-5-(2-((1-methyl-1H-pyrazol-3-yl)amino)-8,9-dihydroimidazo[1′,2′:1,6]pyrido[2,3-d]pyrimidin-6-yl)phenyl)-4-(trifluoromethyl)picolinamide formic acid salt

To a solution of 4-(trifluoromethyl)picolinic acid (40.4 mg, 0.211 mmol), 6-(5-amino-4-fluoro-2-methylphenyl)-N-(1-methyl-1H-pyrazol-3-yl)-8,9-dihydroimidazo[1′,2′:1,6]pyrido[2,3-d]pyrimidin-2-amine (75 mg, 0.192 mmol) and HATU (110 mg, 0.288 mmol) in DMF (7.0 mL) was added DIEA (74 mg, 0.576 mmol). The resulting mixture was stirred at r.t. for 16 h under N₂. The reaction mixture was added into water (40 mL) slowly, stirred at r.t. for 30 min and filtered. The collected cake was purified by column chromatography on silica gel (DCM:MeOH=10:1, +0.1% NH₃-MeOH) to give the crude product, which was purified by Prep-HPLC (0.1% TA) to afford N-(2-fluoro-4-methyl-5-(2-((1-methyl-1H-pyrazol-3-yl)amino)-8,9-dihydroimidazo[1′,2′:1,6]pyrido[2,3-d]pyrimidin-6-yl)phenyl)-4-(trifluoromethyl)picolinamide formic acid salt (30.2 mg, 26% yield) as a yellow solid.

¹H NMR (400 MHz, CD₃OD): δ 9.01 (d, J=4.8 Hz, 1H), 8.98 (s, 1H), 8.46 (s, 1H), 8.44 (s, 1H), 8.16 (s, 1H), 8.14 (s, 1H), 8.05 (d, J=7.6 Hz, 1H), 8.01 (d, J=4.8 Hz, 1H), 7.92 (s, 1H), 7.62 (d, J=2.0 Hz, 1H), 7.38 (d, J=11.6 Hz, 1H), 6.76 (br, 1H), 4.86-4.81 (m, 2H), 4.24 (t, J=10.0 Hz, 2H), 3.88 (s, 3H), 2.30 (s, 3H). LCMS (M+H⁺) m/z: 564.2.

### Example 270: Preparation of 2-fluoro-4-(2-(methylamino)-8,9-dihydroimidazo[1′,2′:1,6]pyrido[2,3-d]pyrimidin-6-yl)-N-(4-(trifluoromethyl)pyridin-2-yl)benzamide (Compound 270)

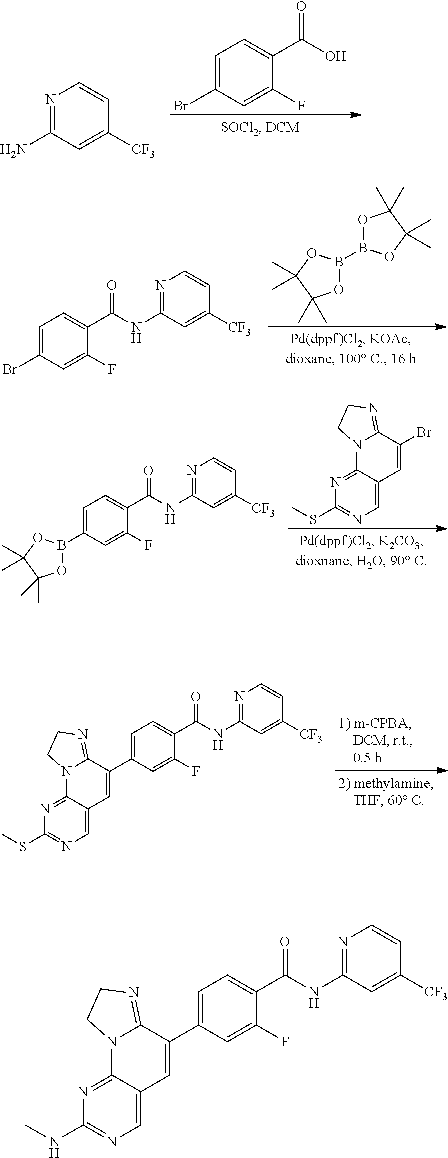

### Step 1: Synthesis of 4-bromo-2-fluoro-N-(4-(trifluoromethyl)pyridin-2-yl)benzamide

To a solution of 4-bromo-2-fluorobenzoic acid (1.1 g, 5 mmol) in DCM (15 mL) was added SOCl₂ (2.38 g, 20 mmol), the reaction mixture was stirred at r.t. for 30 min. The resulting mixture was concentrated to obtain a crude solid. The crude solid was dissolved in DCM (20 mL), then 4-(trifluoromethyl)pyridin-2-amine (810 mg, 5 mmol) and TEA (1.01 g, 10 mmol) was added at 0° C., the reaction mixture was stirred at 25° C. for 16 h. LCMS showed the reaction completed. The reaction mixture was diluted to water (20 mL), extracted by DCM (20 mL*3). The combined organic phase was washed by brine (50 mL), dried over anhydrous Na₂SO₄, concentrated to obtain a white solid, which was purified by chromatography column (PE:EA=4:1) to afford compound 1 (620 mg, 34% yield). LCMS (M+H⁺) m/z: 362.8

### Step 2: Synthesis of 2-fluoro-4-(4,4,5,5-tetramethyl-1,3,2-dioxaborolan-2-yl)-N-(4-(trifluoromethyl)pyridin-2-yl)benzamide

To a solution of 4-bromo-2-fluoro-N-(4-(trifluoromethyl)pyridin-2-yl)benzamide (620 mg, 1.71 mmol) in dioxane (10 mL) was added 4,4,4′,4′,5,5,5′,5′-octamethyl-2,2′-bi(1,3,2-dioxaborolane) (434 mg, 1.71 mmol), KOAc (335 mg, 3.42 mmol) and Pd(dppf)Cl₂ (124 mg, 0.17 mmol) under nitrogen atmosphere. The mixture was stirred at 100° C. for 2 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by chromatography column (PE:EA=2:1) to afford 2-fluoro-4-(4,4,5,5-tetramethyl-1,3,2-dioxaborolan-2-yl)-N-(4-(trifluoromethyl)pyridin-2-yl)benzamide (330 mg, 47% yield) as white solid. LCMS (M+H⁺) m/z: 411.2

### Step 3: Synthesis of 2-fluoro-4-(2-(methylthio)-8,9-dihydroimidazo[1′,2′:1,6]pyrido[2,3-d]pyrimidin-6-yl)-N-(4-(trifluoromethyl)pyridin-2-yl)benzamide

To a solution of 2-fluoro-4-(4,4,5,5-tetramethyl-1,3,2-dioxaborolan-2-yl)-N-(4-(trifluoromethyl)pyridin-2-yl)benzamide (200 mg, 0.48 mmol) in dioxane (4 mL) and H₂O (1 mL) was added 6-bromo-2-(methylthio)-8,9-dihydroimidazo[1′,2′:1,6]pyrido[2,3-d]pyrimidine (142 mg, 0.48 mmol), K₂CO₃ (132 mg, 0.96 mmol) and Pd(dppf)Cl₂ (37 mg, 0.05 mmol) under nitrogen atmosphere. The mixture was stirred at 90° C. for 3 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by chromatography column (PE:EA=1:2) to afford 2-fluoro-4-(2-(methylthio)-8,9-dihydroimidazo[1′,2′:1,6]pyrido[2,3-d]pyrimidin-6-yl)-N-(4-(trifluoromethyl)pyridin-2-yl)benzamide (90 mg, 38% yield) as yellow solid. LCMS (M+H⁺) m/z: 501.2

### Step 4: Synthesis of 2-fluoro-4-(2-(methylamino)-8,9-dihydroimidazo[1′,2′:1,6]pyrido[2,3-d]pyrimidin-6-yl)-N-(4-(trifluoromethyl)pyridin-2-yl)benzamide

To a solution of 2-fluoro-4-(2-(methylthio)-8,9-dihydroimidazo[1′,2′:1,6]pyrido[2,3-d]pyrimidin-6-yl)-N-(4-(trifluoromethyl)pyridin-2-yl)benzamide (90 mg, 0.18 mmol) in DCM (5 mL) was added m-CPBA (62 mg, 0.36 mmol), the reaction mixture was stirred at r.t. for 30 min. LCMS showed the reaction completed. The reaction mixture was concentrated to obtain a yellow solid. To the crude solid in THF (3 mL) was added methylamine in THF (1 mL, 2 mmol). The mixture was stirred at r.t. for 1 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by prep-HPLC to afford 2-fluoro-4-(2-(methylamino)-8,9-dihydroimidazo[1′,2′:1,6]pyrido[2,3-d]pyrimidin-6-yl)-N-(4-(trifluoromethyl)pyridin-2-yl)benzamide (14.4 mg, 17% yield) as yellow solid. ¹H NMR (400 MHz, DMSO-d₆): δ 11.27 (s, 1H), 8.67 (d, J=5.2 Hz, 1H), 8.53 (s, 1H), 8.37-8.32 (m, 1H), 8.04 (d, J=12.8 Hz, 1H), 7.88 (d, J=8.0 Hz, 1H), 7.83 (d, J=6.0 Hz, 1H), 7.75 (t, J=8.0 Hz, 1H), 7.70-7.60 (m, 1H), 7.57 (d, J=5.2 Hz, 1H), 4.12-3.94 (m, 4H), 2.86 (s, 3H). LCMS (M+H⁺) m/z: 484.0.

### Example 271: Preparation of 2-fluoro-5-(2-(methylamino)-8,9-dihydroimidazo[1′,2′:1,6]pyrido[2,3-d]pyrimidin-6-yl)-N-(3-(trifluoromethyl)phenyl)benzamide (Compound 271)

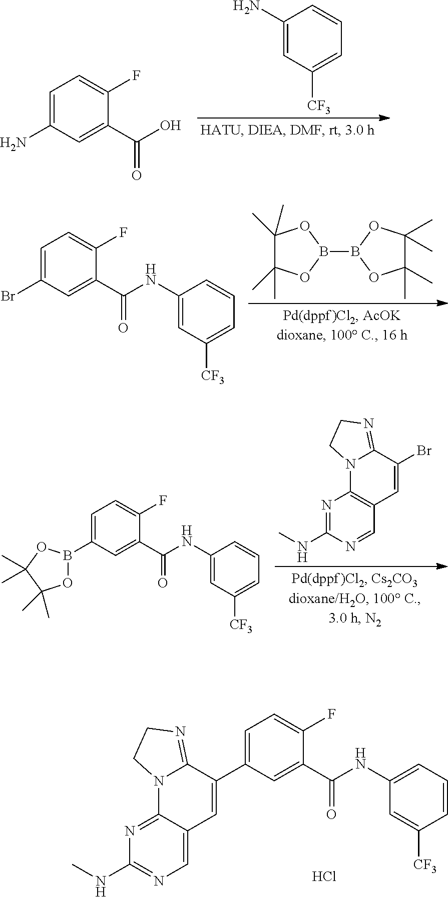

### Step 1: Synthesis of 5-bromo-2-fluoro-N-(3-(trifluoromethyl)phenyl)benzamide

A mixture of 5-bromo-2-fluorobenzoic acid (0.70 g, 3.2 mmol) and O-(7-azabenzotriazol-1-yl)-N,N,N′,N′-tetramethyluronium hexafluorophosphate (1.82 g, 4.8 mmol) in DMF (15.0 mL) was stirred at r.t. for 0.5 h, then 3-(trifluoromethyl)aniline (0.618 g, 3.8 mmol) and N,N-Diisopropylethylamine (1.24 g, 9.6 mmol) was added. The resulting mixture was stirred at r.t. for 2.0 h, then H₂O (50 mL) was added, the reaction mixture was extracted with EA (100 mL×3). The combined organic phases were washed with brine (100 mL), dried with Na₂SO₄, filtered and concentrated. The residue was purified by column chromatography (PE/EA=10/1) to afford 5-bromo-2-fluoro-N-(3-(trifluoromethyl)phenyl)benzamide (980 mg, 85% yield) as a yellow solid. LCMS (M+H⁺) m/z: 361.9 and 363.9.

### Step 2: Synthesis of 2-fluoro-5-(4,4,5,5-tetramethyl-1,3,2-dioxaborolan-2-yl)-N-(3-(trifluoromethyl)phenyl)benzamide

A mixture of 5-bromo-2-fluoro-N-(3-(trifluoromethyl)phenyl)benzamide (1.0 g, 2.70 mmol), 4,4,4′,4′,5,5,5′,5′-octamethyl-2,2′-bi(1,3,2-dioxaborolane) (2.1 g, 8.10 mmol), Pd(dppf)Cl₂ (0.2 g, 0.27 mmol), KOAc (0.8 g, 8.10 mmol) in dioxane (40.0 mL) was charged into a flask, and degassed and charged with Ar three times, and then stirred at 100° C. for 16 hours. The reaction mixture was concentrated. H₂O (50 mL) was added, the mixture was extracted with EA (100 mL×3). The combined organic phases were washed with brine (100 mL), dried with Na₂SO₄, filtered and concentrated. The residue was purified by column chromatography (PE/EA=10/1) to afford 2-fluoro-5-(4,4,5,5-tetramethyl-1,3,2-dioxaborolan-2-yl)-N-(3-(trifluoromethyl)phenyl)benzamide (550 mg, 50% yield) as a brown solid. LCMS (M+H⁺) m/z: 410.0.

### Step 3: Synthesis of 2-fluoro-5-(2-(methylamino)-8,9-dihydroimidazo[1′,2′:1,6]pyrido[2,3-d]pyrimidin-6-yl)-N-(3-(trifluoromethyl)phenyl)benzamide

A mixture of 2-fluoro-5-(4,4,5,5-tetramethyl-1,3,2-dioxaborolan-2-yl)-N-(3-(trifluoromethyl)phenyl)benzamide (0.6 g, 0.54 mmol), 6-bromo-N-methyl-8,9-dihydroimidazo[1′,2′:1,6]pyrido[2,3-d]pyrimidin-2-amine (0.1 g, 0.36 mmol) in dioxane (6.6 mL) and H₂O (0.66 mL), was added Pd(dppf)Cl₂ (0.03 g, 0.04 mmol) and Cs₂CO₃ (0.35 g, 1.1 mmol). The resulting mixture was degassed and charged with N₂ for 3 times, stirred at 100° C. for 3 h. The reaction mixture was concentrated. H₂O (50 mL) was added, the mixture was extracted with EA (50 mL×3). The combined organic phases were washed with brine (100 mL), dried with Na₂SO₄, filtered and concentrated. The residue was purified by Prep-HPLC to afford 2-fluoro-5-(2-(methylamino)-8,9-dihydroimidazo[1′,2′:1,6]pyrido[2,3-d]pyrimidin-6-yl)-N-(3-(trifluoromethyl)phenyl)benzamide (30 mg, 17% yield) as a yellow solid. ¹H NMR (400 MHz, DMSO-d₆): δ 11.37 (s, 1H), 10.51 (s, 1H), 8.88 (s, 1H), 8.55 (d, J=4.8 Hz, 1H), 8.31 (s, 2H), 8.09 (d, J=8.0 Hz, 1H), 7.90-7.88 (m, 1H), 7.76-7.73 (m, 1H), 7.64-7.55 (m, 2H), 7.49 (d, J=7.6 Hz, 1H), 4.65-4.52 (m, 2H), 4.08 (t, J=10.0 Hz, 2H), 2.96 (d, J=4.4 Hz, 3H). LCMS (M+H⁺) m/z: 483.3.

### Example 272: Preparation of N-(3-chlorophenyl)-2-fluoro-5-(2-(methylamino)-8,9-dihydroimidazo[1′,2′:1,6]pyrido[2,3-d]pyrimidin-6-yl)benzamide (Compound 272)

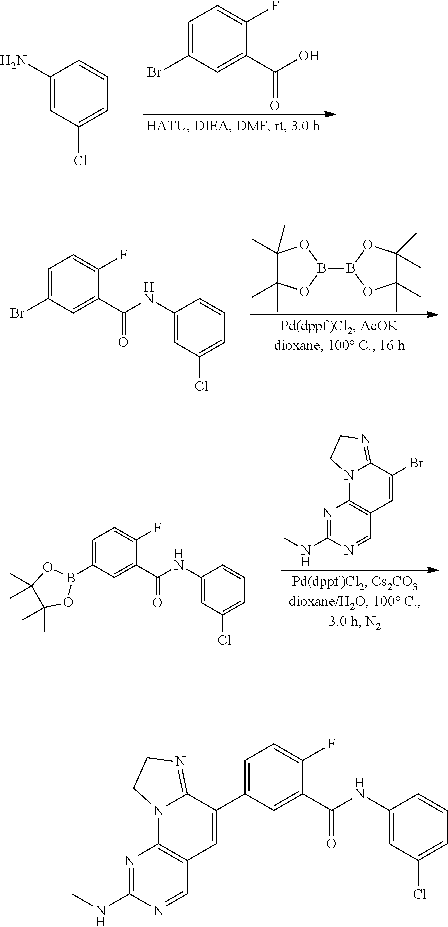

### Step 1: Synthesis of 5-bromo-2-fluoro-N-(3-(trifluoromethyl)phenyl)benzamide

A mixture of 5-bromo-2-fluorobenzoic acid (0.70 g, 3.2 mmol) and O-(7-azabenzotriazol-1-yl)-N,N,N′,N′-tetramethyluronium hexafluorophosphate (1.82 g, 4.8 mmol) in DMF (15.0 mL) was stirred at r.t. for 0.5 h, then 3-chloroaniline (0.49 g, 3.8 mmol) and N,N-diisopropylethylamine (1.24 g, 9.6 mmol) was added. The mixture was stirred at r.t. for 2 h. H₂O (50 mL) was added, extracted with EA (100 mL×3). The combined organic phases were washed with brine (100 mL), dried with Na₂SO₄, filtered and concentrated. The residue was purified by column chromatography (PE/EA=10/1) to afford 5-bromo-2-fluoro-N-(3-(trifluoromethyl)phenyl)benzamide (600 mg, 57% yield) as a yellow solid. LCMS (M+H⁺) m/z: 329.9.

### Step 2: Synthesis of N-(3-chlorophenyl)-2-fluoro-5-(4,4,5,5-tetramethyl-1,3,2-dioxaborolan-2-yl)benzamide

A mixture of 5-bromo-N-(3-chlorophenyl)-2-fluorobenzamide (0.60 g, 1.8 mmol), 4,4,4′,4′,5,5,5′,5′-octamethyl-2,2′-bi(1,3,2-dioxaborolane) (1.40 g, 5.5 mmol), Pd(dppf)Cl₂ (0.13 g, 0.18 mmol), KOAc (0.54 g, 5.5 mmol) in dioxane (18.0 mL) was charged into a flask, degassed and charged with Ar for 3 times. The reaction mixture was stirred at 100° C. for 16 h, then concentrated. H₂O (50 mL) was added, the mixture was extracted with EA (100 mL×3). The combined organic phases were washed with brine (100 mL), dried with Na₂SO₄, filtered and concentrated. The residue was purified by column chromatography (PE/EA=10/1) to afford N-(3-chlorophenyl)-2-fluoro-5-(4,4,5,5-tetramethyl-1,3,2-dioxaborolan-2-yl)benzamide (320 mg, 56% yield) as a brown solid. LCMS (M+H⁺) m/z: 376.1.

### Step 3: Synthesis of N-(3-chlorophenyl)-2-fluoro-5-(2-(methylamino)-8,9-dihydroimidazo[1′,2′:1,6]pyrido[2,3-d]pyrimidin-6-yl)benzamide

A mixture of N-(3-chlorophenyl)-2-fluoro-5-(4,4,5,5-tetramethyl-1,3,2-dioxaborolan-2-yl)benzamide (0.15 g, 0.39 mmol), 6-bromo-N-methyl-8,9-dihydroimidazo[1′,2′:1,6]pyrido[2,3-d]pyrimidin-2-amine (0.10 g, 0.35 mmol) in dioxane (10.0 mL) and H₂O (1.0 mL), were added Pd(dppf)Cl₂ (0.03 g, 0.04 mmol) and Cs₂CO₃ (0.35 g, 1.1 mmol). The mixture was degassed and charged with N₂ for 3 times, stirred at 100° C. for 3 h. The reaction mixture was concentrated. H₂O (50 mL) was added, the mixture was extracted with EA (50 mL×3). The combined organic phases were washed with brine (100 mL), dried with Na₂SO₄, filtered and concentrated. The residue was purified by trituration to afford N-(3-chlorophenyl)-2-fluoro-5-(2-(methylamino)-8,9-dihydroimidazo[1′,2′:1,6]pyrido[2,3-d]pyrimidin-6-yl)benzamide (66 mg, 42% yield) as a yellow solid. ¹H NMR (400 MHz, DMSO-d₆): δ 10.64 (s, 1H), 8.30-8.23 (m, 1H), 8.19-8.17 (m, 1H), 8.15-8.12 (m, 1H), 7.92 (s, 1H), 7.63-7.62 (m, 2H), 7.50-7.46 (m, 1H), 7.42-7.36 (m, 2H), 7.19 (dd, J=8.0, 1.2 Hz, 1H), 4.04-3.96 (s, 4H), 2.84 (s, 3H). LCMS (M+H⁺) m/z: 449.3.

### Example 273: Preparation of 4-(2-(methylamino)-8,9-dihydroimidazo[1′,2′:1,6]pyrido[2,3-d]pyrimidin-6-yl)-N-(4-(trifluoromethyl)pyridin-2-yl)benzamide (Compound 273)

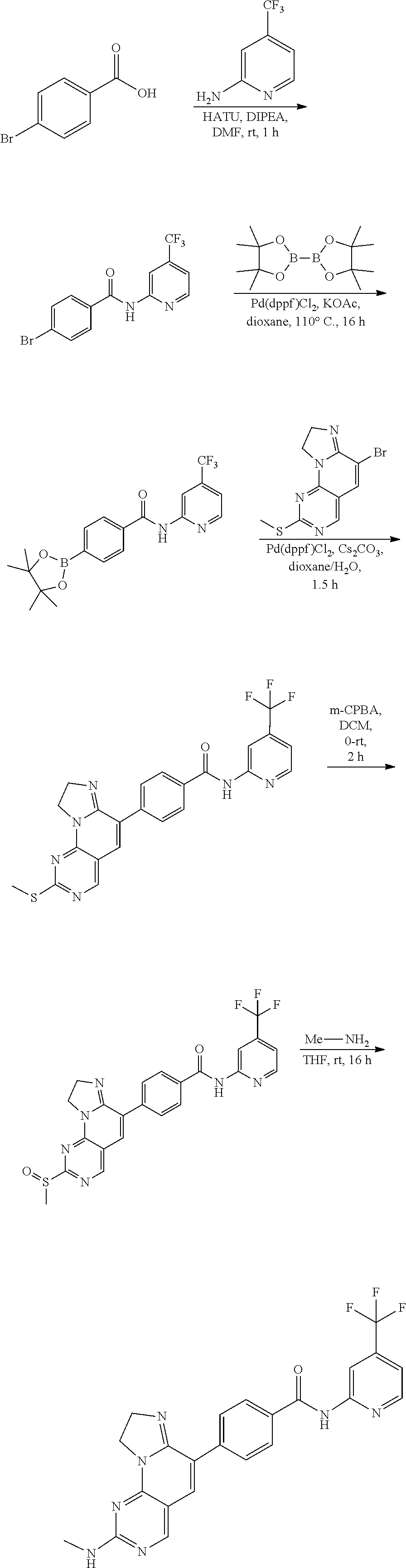

### Step 1: Synthesis of 4-bromo-N-(4-(trifluoromethyl)pyridin-2-yl)benzamide

A mixture of 4-bromobenzoic acid (1.0 g, 4.97 mmol), 4-(trifluoromethyl)pyridin-2-amine (886 mg, 5.47 mmol), DIEA (1.9 g, 14.9 mmol) and HATU (2.83 g, 7.45 mmol) in DMF (15.0 mL) was stirred at r.t. for 1.0 h. The reaction mixture diluted with water (150 mL) and extracted with EA (100 mL×2). The combined organic phase was washed with brine (100 mL×3), dried with Na₂SO₄, filtered and concentrated. The residue was purified by column chromatography (PE/EA=4/1) to afford 4-bromo-N-(4-(trifluoromethyl)pyridin-2-yl)benzamide (1.05 g, 62% yield) as a pale yellow solid. LCMS (M+H⁺) m/z: 345.0 and 347.0.

### Step 2: Synthesis of 4-(4,4,5,5-tetramethyl-1,3,2-dioxaborolan-2-yl)-N-(4-(trifluoromethyl)pyridin-2-yl)benzamide

A mixture of 4-bromo-N-(4-(trifluoromethyl)pyridin-2-yl)benzamide (948 mg, 2.75 mmol), bis(pinacolato)diboron (1.39 g, 5.49 mmol), KOAc (808 mg, 8.25 mmol) and Pd(dppf)Cl₂ (201 mg, 0.275 mmol) in dioxane (25.0 mL) was degassed and charged with N₂ for 3 times and stirred at 110° C. for 16 h. The reaction mixture was concentrated and purified by column chromatography (PE/EA=10/1) to afford 4-(4,4,5,5-tetramethyl-1,3,2-dioxaborolan-2-yl)-N-(4-(trifluoromethyl)pyridin-2-yl)benzamide (900 mg, 83% yield) as a yellow solid. LCMS (M+H⁺) m/z: 393.2.

### Step 3: Synthesis of 4-(2-(methylthio)-8,9-dihydroimidazo[1′,2′:1,6]pyrido[2,3-d]pyrimidin-6-yl)-N-(4-(trifluoromethyl)pyridin-2-yl)benzamide

A mixture of 4-(4,4,5,5-tetramethyl-1,3,2-dioxaborolan-2-yl)-N-(4-(trifluoromethyl)pyridin-2-yl)benzamide (728 mg, 1.86 mmol), 6-bromo-2-(methylthio)-8,9-dihydroimidazo[1′,2′:1,6]pyrido[2,3-d]pyrimidine (500 mg, 1.69 mmol), Cs₂CO₃ (1.65 g, 5.07 mmol) and Pd(dppf)Cl₂ (123 mg, 0.17 mmol) in dioxane (20.0 mL) and water (5.0 mL) was degassed and charged with N₂ for 3 times and stirred at 85° C. for 1.5 h. The reaction mixture was concentrated and purified by column chromatography (PE/EA=1/2, +0.1% TEA) to afford 4-(2-(methylthio)-8,9-dihydroimidazo[1′,2′:1,6]pyrido[2,3-d]pyrimidin-6-yl)-N-(4-(trifluoromethyl)pyridin-2-yl)benzamide (793 mg, 88% yield) as a yellow solid. LCMS (M+H⁺) m/z: 482.9.

### Step 4: Synthesis of 4-(2-(methylsulfinyl)-8,9-dihydroimidazo[1′,2′:1,6]pyrido[2,3-d]pyrimidin-6-yl)-N-(4-(trifluoromethyl)pyridin-2-yl)benzamide

To a solution of 4-(2-(methylthio)-8,9-dihydroimidazo[1′,2′:1,6]pyrido[2,3-d]pyrimidin-6-yl)-N-(4-(trifluoromethyl)pyridin-2-yl)benzamide (150 mg, 0.3 mmol) in dry DCM (15 mL) was added m-CPBA (135 mg, 0.77 mmol) at 0° C. The resulting mixture was stirred at 0° C. for 30 min. The reaction mixture was concentrated at r.t. in vacuum to afford crude 4-(2-(methylsulfinyl)-8,9-dihydroimidazo[1′,2′:1,6]pyrido[2,3-d]pyrimidin-6-yl)-N-(4-(trifluoromethyl)pyridin-2-yl)benzamide (150 mg, crude) as a yellow solid, which was used in the next step without purification. LCMS (M+H⁺) m/z: 499.0.

### Step 5: Synthesis of 4-(2-(methylamino)-8,9-dihydroimidazo[1′,2′:1,6]pyrido[2,3-d]pyrimidin-6-yl)-N-(4-(trifluoromethyl)pyridin-2-yl)benzamide

To a solution of crude 4-(2-(methylsulfinyl)-8,9-dihydroimidazo[1′,2′:1,6]pyrido[2,3-d]pyrimidin-6-yl)-N-(4-(trifluoromethyl)pyridin-2-yl)benzamide (150 mg, 0.30 mmol) in THE (15 mL) was added MeNH₂ (2 M in THF, 0.6 mL, 1.2 mmol). The mixture was stirred at r.t. for 16 h and concentrated. Water (80 mL) was added, the mixture was extracted with EA (50 mL×3). The combined organic phase was washed with brine (100 mL), dried with Na₂SO₄, filtered and concentrated. The residue was purified by Prep-HPLC (0.1% NH₃—H₂O) to afford 4-(2-(methylamino)-8,9-dihydroimidazo[1′,2′:1,6]pyrido[2,3-d]pyrimidin-6-yl)-N-(4-(trifluoromethyl)pyridin-2-yl)benzamide (46 mg, 32% yield) as a yellow solid. ¹H NMR (400 MHz, DMSO-d₆): δ 11.28 (s, 1H), 8.69 (d, J=5.2 Hz, 1H), 8.57 (s, 1H), 8.33-8.27 (m, 1H), 8.09-8.04 (m, 4H), 7.68 (s, 1H), 7.55-7.49 (m, 2H), 4.02-4.04 (m, 4H), 2.85 (s, 3H). LCMS (M+H⁺) m/z: 466.3.

### Example 274: Preparation of N-(2-fluoro-5-(trifluoromethyl)phenyl)-4-(2-(methylamino)-8,9-dihydroimidazo[1′,2′:1,6]pyrido[2,3-d]pyrimidin-6-yl)benzamide (Compound 274)

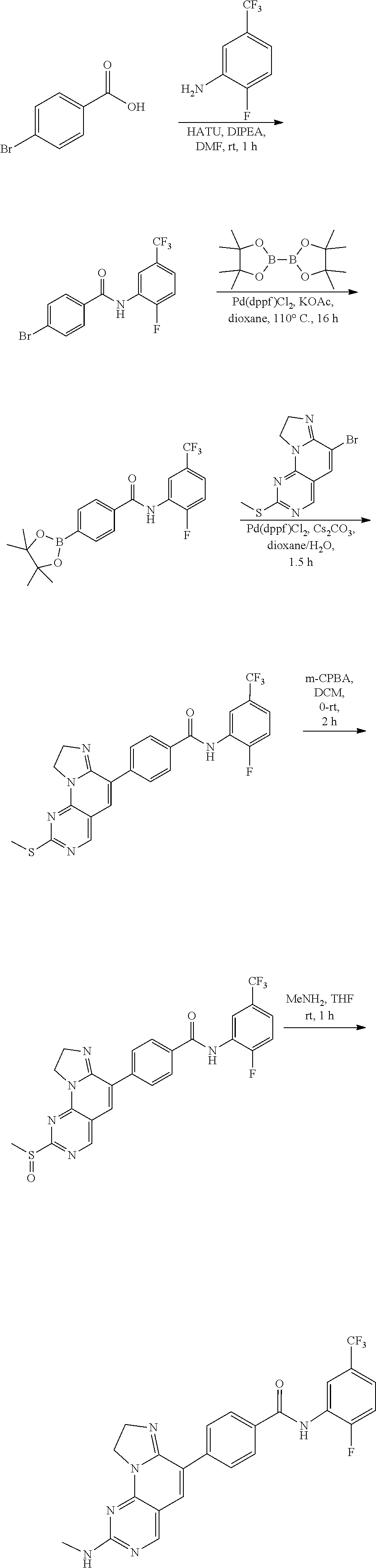

### Step 1: Synthesis of 4-bromo-N-(2-fluoro-5-(trifluoromethyl)phenyl)benzamide

A mixture of 4-bromobenzoic acid (2.0 g, 9.95 mmol), 2-fluoro-5-(trifluoromethyl)aniline (1.96 g, 10.9 mmol), DIEA (3.8 g, 29.8 mmol) and HATU (5.67 g, 14.9 mmol) in DMF (25.0 mL) was stirred at r.t. for 1.0 h. The reaction mixture diluted with water (150 mL) and extracted with EA (100 mL×2). The combined organic phase was washed with brine (100 mL×3), dried with Na₂SO₄, filtered and concentrated. The residue was purified by column chromatography (PE/EA=4/1) to afford 4-bromo-N-(2-fluoro-5-(trifluoromethyl)phenyl)benzamide (2.79 g, 77% yield) as a pale yellow solid. LCMS (M+H⁺) m/z: 361.8

### Step 2: Synthesis of N-(2-fluoro-5-(trifluoromethyl)phenyl)-4-(4,4,5,5-tetramethyl-1,3,2-dioxaborolan-2-yl)benzamide

A mixture of 4-bromo-N-(2-fluoro-5-(trifluoromethyl)phenyl)benzamide (2.68 g, 7.4 mmol), bis(pinacolato)diboron (3.7 g, 14.8 mmol), KOAc (2.17 g, 22.2 mmol) and Pd(dppf)Cl₂ (540 mg, 0.74 mmol) in dioxane (35.0 mL) was degassed and charged with N₂ for 3 times and stirred at 110° C. for 16 h. The reaction mixture was concentrated and purified by column chromatography (PE/EA=10/1) to afford N-(2-fluoro-5-(trifluoromethyl)phenyl)-4-(4,4,5,5-tetramethyl-1,3,2-dioxaborolan-2-yl)benzamide (2.5 g, 83% yield) as a yellow solid. LCMS (M+H⁺) m/z: 410.2.

### Step 3: Synthesis of N-(2-fluoro-5-(trifluoromethyl)phenyl)-4-(2-(methylthio)-8,9-dihydroimidazo[1′,2′:1,6]pyrido[2,3-d]pyrimidin-6-yl)benzamide

A mixture of N-(2-fluoro-5-(trifluoromethyl)phenyl)-4-(4,4,5,5-tetramethyl-1,3,2-dioxaborolan-2-yl)benzamide (1.2 g, 1.86 mmol), 6-bromo-2-(methylthio)-8,9-dihydroimidazo[1′,2′:1,6]pyrido[2,3-d]pyrimidine (500 mg, 1.69 mmol), Cs₂CO₃ (1.65 g, 5.07 mmol) and Pd(dppf)Cl₂ (123 mg, 0.17 mmol) in dioxane (20.0 mL) and water (5.0 mL) was degassed and charged with N₂ for 3 times and stirred at 85° C. for 1.5 h. The reaction mixture was concentrated and purified by column chromatography (PE/EA=1/2, +0.1% TEA) to afford N-(2-fluoro-5-(trifluoromethyl)phenyl)-4-(2-(methylthio)-8,9-dihydroimidazo[1′,2′:1,6]pyrido[2,3-d]pyrimidin-6-yl)benzamide (730 mg, 52% yield) as a yellow solid. LCMS (M+H⁺) m/z: 500.0.

### Step 4: Synthesis of N-(2-fluoro-5-(trifluoromethyl)phenyl)-4-(2-(methylsulfinyl)-8,9-dihydroimidazo[1′,2′:1,6]pyrido[2,3-d]pyrimidin-6-yl)benzamide

To a solution of N-(2-fluoro-5-(trifluoromethyl)phenyl)-4-(2-(methylthio)-8,9-dihydroimidazo[1′,2′:1,6]pyrido[2,3-d]pyrimidin-6-yl)benzamide (150 mg, 0.3 mmol) in dry DCM (15 mL) was added m-CPBA (156 mg, 0.9 mmol) at 0° C. The resulting mixture was stirred at 0° C. for 30 min. The reaction mixture was concentrated at r.t. to afford crude N-(2-fluoro-5-(trifluoromethyl)phenyl)-4-(2-(methylsulfinyl)-8,9-dihydroimidazo[1′,2′:1,6]pyrido[2,3-d]pyrimidin-6-yl)benzamide (150 mg, crude) as a yellow solid, which was used in the next step without purification. LCMS (M+H⁺) m/z: 516.0.

### Step 5: Synthesis of N-(2-fluoro-5-(trifluoromethyl)phenyl)-4-(2-(methylamino)-8,9-dihydroimidazo[1′,2′:1,6]pyrido[2,3-d]pyrimidin-6-yl)benzamide

To a solution of crude N-(2-fluoro-5-(trifluoromethyl)phenyl)-4-(2-(methylsulfinyl)-8,9-dihydroimidazo[1′,2′:1,6]pyrido[2,3-d]pyrimidin-6-yl)benzamide (150 mg, 0.28 mmol) in THE (15 mL) was added MeNH₂ (2 M in THF, 0.56 mL, 1.13 mmol). The mixture was stirred at r.t. for 16 h and concentrated. water (80 mL) was added and the mixture was extracted with EA (50 mL×3). The combined organic phase was washed with brine (100 mL), dried with Na₂SO₄, filtered and concentrated. The residue was purified by Prep-HPLC (0.1% NH₃—H₂O) to afford N-(2-fluoro-5-(trifluoromethyl)phenyl)-4-(2-(methylamino)-8,9-dihydroimidazo[1′,2′:1,6]pyrido[2,3-d]pyrimidin-6-yl)benzamide (53.5 mg, 38% yield) as a yellow solid. ¹H NMR (400 MHz, DMSO-d₆): δ 10.36 (s, 1H), 8.34-8.28 (m, 1H), 8.12-8.06 (m, 3H), 8.00 (d, J=8.4 Hz, 2H), 7.68 (s, 2H), 7.60-7.51 (m, 2H), 4.07-3.98 (m, 4H), 2.86 (s, 3H). LCMS (M+H⁺) m/z: 483.3.

### Example 275: Preparation of N-(2-chlorophenyl)-4-(2-(methylamino)-8,9-dihydroimidazo[1′,2′:1,6]pyrido[2,3-d]pyrimidin-6-yl)benzamide (Compound 275)

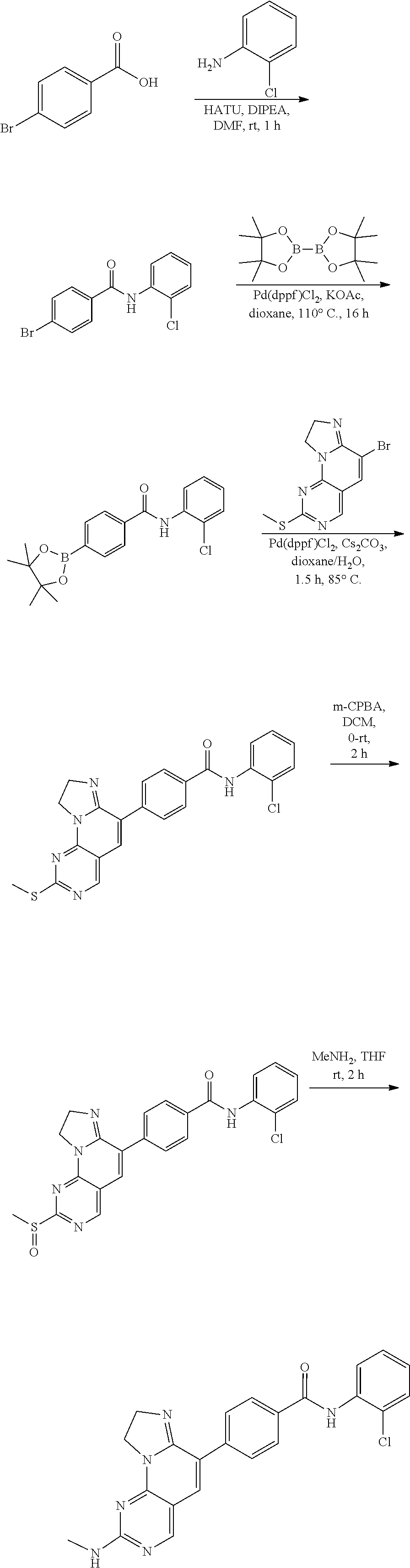

### Step 1: Synthesis of 4-bromo-N-(2-chlorophenyl)benzamide

A mixture of 4-bromobenzoic acid (2.0 g, 9.95 mmol), 2-chloroaniline (1.39 g, 10.9 mmol), DIEA (4.2 g, 32.8 mmol) and HATU (6.2 g, 16.4 mmol) in DMF (25.0 mL) was stirred at r.t. for 1 h. The reaction mixture diluted with water (150.0 mL) and extracted with EA (100 mL×2). The combined organic phase was washed with brine (100 mL×3), dried with Na₂SO₄, filtered and concentrated. The residue was purified by column chromatography (PE/EA=5/1) to afford 4-bromo-N-(2-chlorophenyl)benzamide (2.6 g, 86% yield) as a pale yellow solid. LCMS (M+H⁺) m/z: 310.0 and 312.0.

### Step 2: Synthesis of N-(2-chlorophenyl)-4-(4,4,5,5-tetramethyl-1,3,2-dioxaborolan-2-yl)benzamide

A mixture of 4-bromo-N-(2-chlorophenyl)benzamide (2.5 g, 8.06 mmol), bis(pinacolato)diboron (4.09 g, 16.12 mmol), KOAc (2.36 g, 24.18 mmol) and Pd(dppf)Cl₂ (589 mg, 0.806 mmol) in dioxane (35.0 mL) was degassed and charged with N₂ for 3 times and stirred at 110° C. for 16 h. The reaction mixture was concentrated and purified by column chromatography (PE/EA=10/1) to afford N-(2-chlorophenyl)-4-(4,4,5,5-tetramethyl-1,3,2-dioxaborolan-2-yl)benzamide (2.3 g, 82% yield) as a yellow solid. LCMS (M+H⁺) m/z: 358.3.

### Step 3: Synthesis of N-(2-chlorophenyl)-4-(2-(methylthio)-8,9-dihydroimidazo[1′,2′:1,6]pyrido[2,3-d]pyrimidin-6-yl)benzamide

A mixture of N-(2-chlorophenyl)-4-(4,4,5,5-tetramethyl-1,3,2-dioxaborolan-2-yl)benzamide (663 mg, 1.86 mmol), 6-bromo-2-(methylthio)-8,9-dihydroimidazo[1′,2′:1,6]pyrido[2,3-d]pyrimidine (500 mg, 1.69 mmol), Cs₂CO₃ (1.65 g, 5.07 mmol) and Pd(dppf)Cl₂ (123 mg, 0.17 mmol) in dioxane (20.0 mL) and water (5.0 mL) was degassed and charged with N₂ for 3 times and stirred at 85° C. for 1.5 h. The reaction mixture was concentrated and purified by column chromatography (PE/EA=1/2, +0.1% TEA) to afford N-(2-chlorophenyl)-4-(2-(methylthio)-8,9-dihydroimidazo[1′,2′:1,6]pyrido[2,3-d]pyrimidin-6-yl)benzamide (612 mg, 73% yield) as a yellow solid. LCMS (M+H⁺) m/z: 447.9.

### Step 4: Synthesis of N-(2-chlorophenyl)-4-(2-(methylsulfinyl)-8,9-dihydroimidazo[1′,2′:1,6]pyrido[2,3-d]pyrimidin-6-yl)benzamide

To a solution of N-(2-chlorophenyl)-4-(2-(methylthio)-8,9-dihydroimidazo[1′,2′:1,6]pyrido[2,3-d]pyrimidin-6-yl)benzamide (316 mg, 0.7 mmol) in dry DCM (15 mL) was added m-CPBA (367 mg, 2.12 mmol) at 0° C. The resulting mixture was stirred at 0° C. for 30 min. The reaction mixture was concentrated at r.t to afford crude N-(2-chlorophenyl)-4-(2-(methylsulfinyl)-8,9-dihydroimidazo[1′,2′:1,6]pyrido[2,3-d]pyrimidin-6-yl)benzamide (680 mg, crude) as a yellow solid, which was used in the next step without purification. LCMS (M+H⁺) m/z: 464.0.

### Step 5: Synthesis of N-(2-chlorophenyl)-4-(2-(methylamino)-8,9-dihydroimidazo[1′,2′:1,6]pyrido[2,3-d]pyrimidin-6-yl)benzamide

To a solution of crude N-(2-chlorophenyl)-4-(2-(methylsulfinyl)-8,9-dihydroimidazo[1′,2′:1,6]pyrido[2,3-d]pyrimidin-6-yl)benzamide (680 mg, 1.46 mmol) in THE (15 mL) was added MeNH₂ (2 M in THF, 1.4 mL, 2.85 mmol). The mixture was stirred at r.t. for 16 h and concentrated. Water (80 mL) was added and the mixture was extracted with EA (50 mL×3). The combined organic phase was washed with brine (100 mL), dried with Na₂SO₄, filtered and concentrated. The residue was purified by Prep-HPLC (0.1% NH₃—H₂O) to afford N-(2-chlorophenyl)-4-(2-(methylamino)-8,9-dihydroimidazo[1′,2′:1,6]pyrido[2,3-d]pyrimidin-6-yl)benzamide (37.9 mg, 3.1% yield) as a yellow solid. ¹H NMR (400 MHz, DMSO-d₆): δ 10.05 (s, 1H), 8.33-8.26 (m, 1H), 8.06 (d, J=8.0 Hz, 2H), 8.0 (d, J=8.8 Hz, 2H), 7.65 (s, 1H), 7.61 (dd, J=7.6, 1.6 Hz, 1H), 7.57 (dd, J=8.0, 1.6 Hz, 1H), 7.50 (s, 1H), 7.40 (td, J=8.0, 1.6 Hz, 1H) 7.30 (td, J=7.6, 1.6 Hz, 1H), 4.04-4.01 (m, 4H), 2.85 (s, 3H). LCMS (M+H⁺) m/z: 431.3.

### Example 276: Preparation of 3-methyl-4-(2-(methylamino)-8,9-dihydroimidazo[1′,2′:1,6]pyrido[2,3-d]pyrimidin-6-yl)-N-(4-(trifluoromethyl)pyridin-2-yl)benzamide (Compound 276)

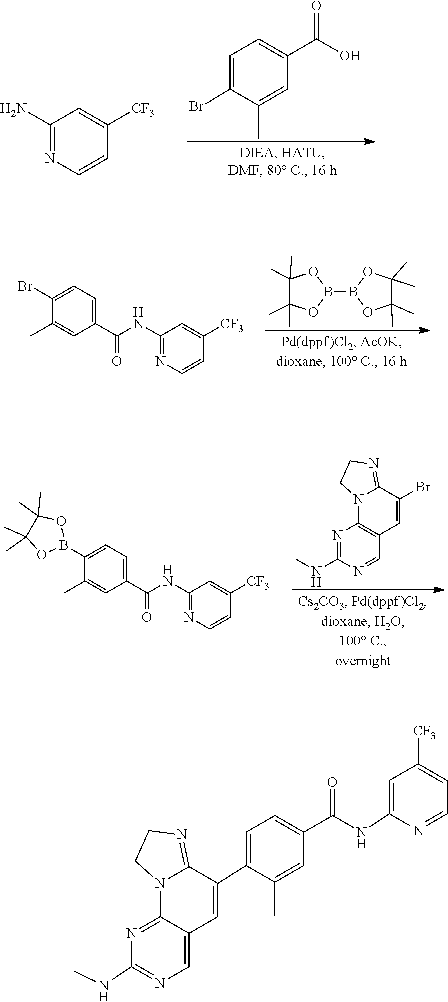

### Step 1: Synthesis of 4-bromo-3-methyl-N-(4-(trifluoromethyl)pyridin-2-yl)benzamide

A mixture of 4-(trifluoromethyl)pyridin-2-amine (500 mg, 3.09 mmol), 4-bromo-3-methylbenzoic acid (660 mg, 3.09 mmol), HATU (1.76 g, 4.63 mmol) and DIEA (1.19 g, 9.26 mmol) in DMF (10.0 mL) was stirred at 80° C. for 16 h. The reaction was cooled to r.t. and diluted with water (50.0 mL), extracted with EA (80 mL×3). The combined organic phase was washed with brine (50 mL×3), dried over Na₂SO₄, filtered and concentrated and purified by column chromatography (PE/EA=10/1) to afford 4-bromo-3-methyl-N-(4-(trifluoromethyl)pyridin-2-yl)benzamide (670 mg, 60% yield) as a yellow solid. LCMS (M+H⁺) m/z: 359.0 and 361.0.

### Step 2: Synthesis of (3-methyl-4-(4,4,5,5-tetramethyl-1,3,2-dioxaborolan-2-yl)-N-(4-(trifluoromethyl) pyridin-2-yl)benzamide

To a solution of 4-bromo-3-methyl-N-(4-(trifluoromethyl)pyridin-2-yl)benzamide (670 mg, 1.87 mmol) Bis(pinacolato)diboron (529 mg, 2.24 mmol), KOAc (548 mg, 5.6 mmol) in dioxane (10 mL) was added Pd(dppf)Cl₂ (300 mg, 0.4 mmol). The mixture was degassed and charged with N₂ three times, stirred at 100° C. for 16 h. The reaction was cooled to r.t., concentrated and purified by column chromatography (PE/EA=10/1) to afford (3-methyl-4-(4,4,5,5-tetramethyl-1,3,2-dioxaborolan-2-yl)-N-(4-(trifluoromethyl) pyridin-2-yl)benzamide (500 mg, 66% yield) as a yellow solid. LCMS (M+H⁺) m/z: 407.1.

### Step 3: Synthesis of 3-methyl-4-(2-(methylamino)-8,9-dihydroimidazo[1′,2′:1,6]pyrido[2,3-d]pyrimidin-6-yl)-N-(4-(trifluoromethyl)pyridin-2-yl)benzamide formic acid

A mixture of 6-bromo-N-methyl-8,9-dihydroimidazo[1′,2′:1,6]pyrido[2,3-d]pyrimidin-2-amine (150 mg, 0.54 mmol), 3-methyl-4-(4,4,5,5-tetramethyl-1,3,2-dioxaborolan-2-yl)-N-(4-(trifluoromethyl) pyridin-2-yl)benzamide (240 mg, 0.59 mmol), Cs₂CO₃ (524 mg, 1.6 mmol) and Pd(dppf)Cl₂ (40 mg, 0.06 mmol) in dioxane (10 mL) and water (1 mL) was degassed and charged with N₂ three times and stirred at 100° C. for 16 h. The reaction mixture was concentrated and purified by Prep-HPLC (0.1% FA) to afford 3-methyl-4-(2-(methylamino)-8,9-dihydroimidazo[1′,2′:1,6]pyrido[2,3-d]pyrimidin-6-yl)-N-(4-(trifluoromethyl)pyridin-2-yl)benzamide formic acid (7.0 mg, 3% yield) as a yellow solid. ¹H NMR (400 MHz, DMSO-d₆): δ 11.26 (s, 1H), 8.68 (d, J=5.2 Hz, 1H), 8.57 (s, 1H), 8.36 (s, 1H), 8.25-8.18 (m, 1H), 7.95 (s, 1H), 7.87 (d, J=8.0 Hz, 1H), 7.54 (d, J=4.8 Hz, 1H), 7.42 (d, J=8.8 Hz, 1H), 7.36 (d, J=8.0 Hz, 1H), 7.17 (s, 1H), 4.03-3.89 (m, 4H), 2.84 (s, 3H), 2.31 (s, 3H). LCMS (M+H⁺) m/z: 480.3.

### Example 277: Preparation of 2-fluoro-4-methyl-5-(2-(methylamino)-8,9-dihydroimidazo[1′,2′:1,6]pyrido[2,3-d]pyrimidin-6-yl)-N-(4-(trifluoromethyl)pyridin-2-yl)benzamide (Compound 277)

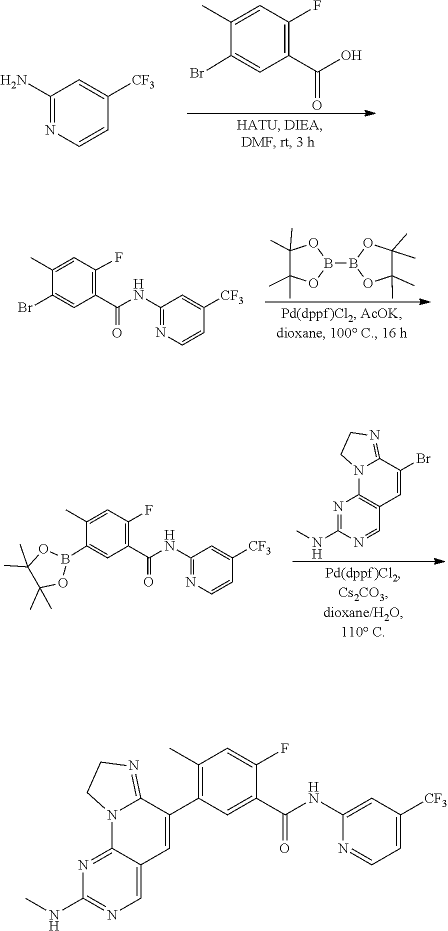

### Step 1: Synthesis of 5-bromo-2-fluoro-4-methyl-N-(4-(trifluoromethyl)pyridin-2-yl)benzamide

A mixture of 5-bromo-2-fluoro-4-methylbenzoic acid (546 mg, 2.35 mmol), 4-bromo-3-methylbenzoic acid (380 mg, 2.34 mmol), HATU (982 mg, 2.56 mmol) and DIEA (455 mg, 3.52 mmol) in DMF (10 mL) was stirred at 80° C. for 16 h. The reaction was cooled to r.t. and diluted with water (50 mL), extracted with EA (80 mL×3). The combined organic phase was washed with brine (50 mL×3), dried over Na₂SO₄, filtered and concentrated and purified by column chromatography (PE/EA=10/1) to afford 5-bromo-2-fluoro-4-methyl-N-(4-(trifluoromethyl)pyridin-2-yl)benzamide (530 mg, 60% yield) as a yellow solid. LCMS (M+H⁺) m/z: 377.0 and 379.0.

### Step 2: 2-fluoro-4-methyl-5-(4,4,5,5-tetramethyl-1,3,2-dioxaborolan-2-yl)-N-(4-(trifluoromethyl)pyridin-2-yl)benzamide

To a solution of 5-bromo-2-fluoro-4-methyl-N-(4-(trifluoromethyl)pyridin-2-yl)benzamide (200 mg, 0.53 mmol), bis(pinacolato)diboron (150 mg, 0.64 mmol), KOAc (156 mg, 1.59 mmol) in dioxane (5 mL) was added Pd(dppf)Cl₂ (77 mg, 0.11 mmol). The mixture was degassed and charged with N₂ three times, stirred at 100° C. for 16 h. The reaction was cooled to r.t., concentrated and purified by column chromatography (PE/EA=10/1) to afford 2-fluoro-4-methyl-5-(4,4,5,5-tetramethyl-1,3,2-dioxaborolan-2-yl)-N-(4-(trifluoromethyl)pyridin-2-yl)benzamide (120 mg, 53% yield) as a yellow solid. LCMS (M+H⁺) m/z: 425.1

### Step 3: Synthesis of 2-fluoro-4-methyl-5-(2-(methylamino)-8,9-dihydroimidazo[1′,2′:1,6]pyrido[2,3-d]pyrimidin-6-yl)-N-(4-(trifluoromethyl)pyridin-2-yl)benzamide

A mixture of 6-bromo-N-methyl-8,9-dihydroimidazo[1′,2′:1,6]pyrido[2,3-d]pyrimidin-2-amine (110 mg, 0.39 mmol), 2-fluoro-4-methyl-5-(4,4,5,5-tetramethyl-1,3,2-dioxaborolan-2-yl)-N-(4-(trifluoromethyl)pyridin-2-yl)benzamide (180 mg, 0.42 mmol), Cs₂CO₃ (250 mg, 0.78 mmol) and Pd(dppf)Cl₂ (32 mg, 0.04 mmol) in dioxane (15.0 mL) and water (3 mL) was degassed and charged and charged with N₂ three times and stirred at 100° C. for 16 h. The reaction mixture was cooled to r.t., diluted with water (20 mL) and extracted with EA (50 mL×3). The combined organic phase was washed with brine (100 mL), dried with Na₂SO₄, filtered and concentrated. The residue was purified by prep-TLC to afford 2-fluoro-4-methyl-5-(2-(methylamino)-8,9-dihydroimidazo[1′,2′:1,6]pyrido[2,3-d]pyrimidin-6-yl)-N-(4-(trifluoromethyl)pyridin-2-yl)benzamide (20 mg, 10% yield) as a yellow solid. ¹H NMR (400 MHz, DMSO-d₆): δ 11.24 (s, 1H), 8.65 (d, J=5.2 Hz, 1H), 8.50 (s, 1H), 8.25 (d, J=9.6 Hz, 1H), 7.58-7.23 (m, 5H), 4.08-3.89 (m, 4H), 2.85 (s, 3H), 2.30 (s, 3H). LCMS (M+H⁺) m/z: 498.1.

### Example 278: Preparation of 2-fluoro-5-(2-(methylamino)-8,9-dihydroimidazo[1′,2′:1,6]pyrido[2,3-d]pyrimidin-6-yl)-N-(4-(trifluoromethyl)pyridin-2-yl)benzamide (Compound 278)

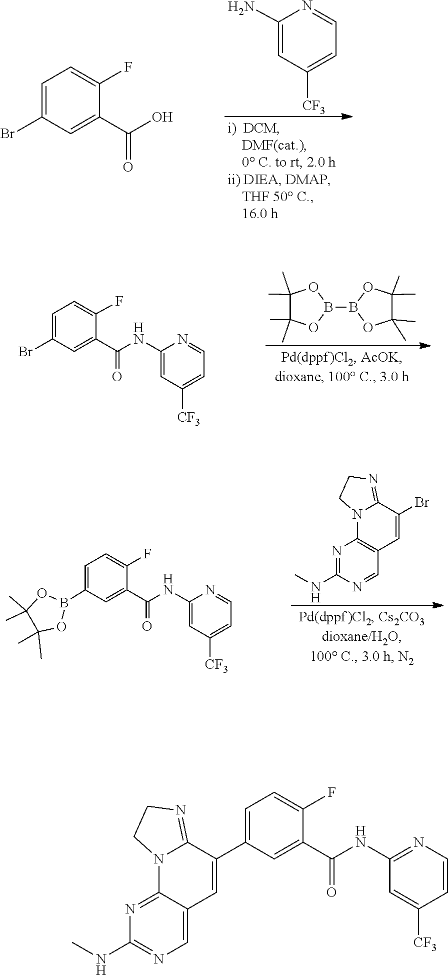

### Step 1: Synthesis of 5-bromo-2-fluoro-N-(4-(trifluoromethyl)pyridin-2-yl)benzamide

To a solution of 5-bromo-2-fluorobenzoic acid (0.5 g, 2.3 mmol) and one drop of DMF in DCM (8.0 mL), oxalyl chloride was added drop-wise at 0° C. The reaction mixture was stirred for 2 h and concentrated in vacuum to remove solvent and used in the next step without further purification. A mixture of 5-bromo-2-fluorobenzoyl chloride (0.5 g, 2.3 mmol), DMAP (0.03 g, 0.23 mmol) in anhydrous THF (6.0 mL), 4-(trifluoromethyl)pyridin-2-amine (0.4 g, 2.4 mmol) and N,N-Diisopropylethylamine (0.4 g, 2.7 mmol) were subsequently added. The reaction mixture was stirred for 16 h and concentrated. H₂O (50 mL) was added, the mixture was extracted with EA (100 mL×3). The combined organic phases were washed with brine (100.0 mL), dried with Na₂SO₄, filtered and concentrated. The residue was purified by column chromatography (PE/EA=3/1) to afford 5-bromo-2-fluoro-N-(4-(trifluoromethyl)pyridin-2-yl)benzamide (566 mg, 68% yield) as a yellow solid. LCMS (M+H⁺) m/z: 362.9.

### Step 2: Synthesis of 2-fluoro-5-(4,4,5,5-tetramethyl-1,3,2-dioxaborolan-2-yl)-N-(4-(trifluoromethyl)pyridine-2-yl)benzamide

A mixture of 5-bromo-2-fluoro-N-(4-(trifluoromethyl)pyridin-2-yl)benzamide (0.3 g, 0.8 mmol), 4,4,4′,4′,5,5,5′,5′-octamethyl-2,2′-bi(1,3,2-dioxaborolane) (0.6 g, 2.5 mmol), KOAc (0.2 g, 2.5 mmol) in dioxane (9.0 mL) was added Pd(dppf)Cl₂ (0.06 g, 0.1 mmol). The resulting mixture was degassed and charged with Ar three times, stirred at 100° C. for 3 h. The reaction mixture was concentrated. H₂O (50.0 mL) was added, the mixture was extracted with EA (100 mL×3). The combined organic phases were washed with brine (100.0 mL), dried with Na₂SO₄, filtered and concentrated. The residue was purified by column chromatography (PE/EA=10/1) to afford 2-fluoro-5-(4,4,5,5-tetramethyl-1,3,2-dioxaborolan-2-yl)-N-(4-(trifluoromethyl)pyridine-2-yl)benzamide (192 mg, 57% yield) as a brown solid. LCMS (M+H⁺) m/z: 410.9.

### Step 3: Synthesis of 2-fluoro-5-(2-(methylamino)-8,9-dihydroimidazo[1′,2′:1,6]pyrido[2,3-d]pyrimidin-6-yl)-N-(4-(trifluoromethyl)pyridin-2-yl)benzamide

To a mixture of 2-fluoro-5-(4,4,5,5-tetramethyl-1,3,2-dioxaborolan-2-yl)-N-(4-(trifluoromethyl)pyridin-2-yl)benzamide (0.2 g, 0.5 mmol), 6-bromo-N-methyl-8,9-dihydroimidazo[1′,2′:1,6]pyrido[2,3-d]pyrimidin-2-amine (0.14 g, 0.5 mmol) in dioxane (13.0 mL) and H₂O (1.3 mL) were added Pd(dppf)Cl₂ (0.04 g, 0.05 mmol), Cs₂CO₃ (0.5 g, 1.4 mmol). The resulting mixture was degassed and charged with N₂ three times, stirred at 100° C. for 3 hrs. The reaction mixture was concentrated. H₂O (50 mL) was added, the mixture was extracted with EA (50 mL×3). The combined organic phases were washed with brine (100 mL), dried with Na₂SO₄, filtered and concentrated. The residue was purified by trituration to afford 2-fluoro-5-(2-(methylamino)-8,9-dihydroimidazo[1′,2′:1,6]pyrido[2,3-d]pyrimidin-6-yl)-N-(4-(trifluoromethyl)pyridin-2-yl)benzamide (43 mg, 20% yield) as a yellow solid. ¹H NMR (400 MHz, DMSO-d₆): δ 11.36 (s, 1H), 8.67 (d, J=4.8 Hz, 1H), 8.53 (s, 1H), 8.30-8.23 (m, 2H), 8.15-8.12 (m, 1H), 7.63 (s, 1H), 7.56 (d, J=4.8 Hz, 1H), 7.48-7.45 (m, 1H), 7.36 (t, J=9.6 Hz, 1H), 4.07-3.97 (m, 4H), 2.84 (s, 3H). LCMS (M+H⁺) m/z: 484.3.

### Example 279: Preparation of 2-fluoro-4-methyl-5-(2-(methylamino)-8,9-dihydroimidazo[1′,2′:1,6]pyrido[2,3-d]pyrimidin-6-yl)-N-(3-(trifluoromethyl)phenyl)benzamide (Compound 279)

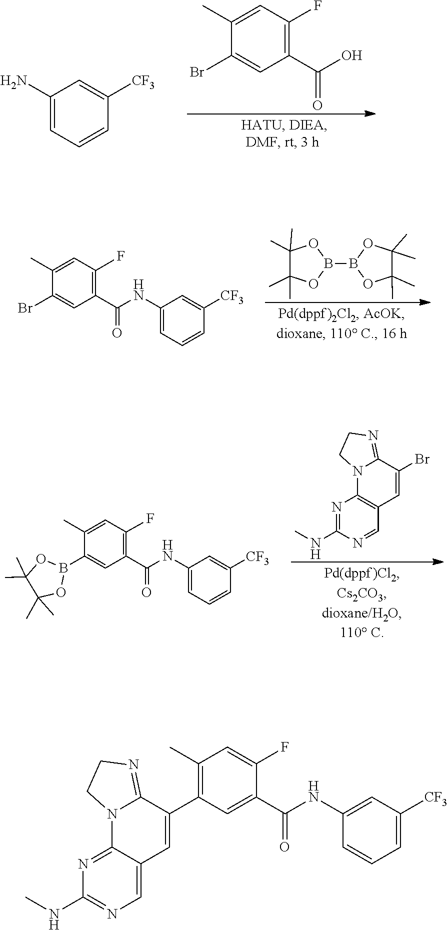

### Step 1: Synthesis of 5-bromo-2-fluoro-4-methyl-N-(3-(trifluoromethyl)phenyl)benzamide

A mixture of 5-bromo-2-fluoro-4-methylbenzoic acid (466 mg. 2 mmol), 3-(trifluoromethyl)aniline (354 mg, 2.2 mmol), HATU (836 mg, 2.2 mmol) and DIEA (390 mg, 3 mmol) in DMF (30 mL) at RT. The reaction mixture was stirred at RT for 2 h. The reaction mixture was diluted with water (80 mL), extracted with EA (100 mL×3). The combined organic phase was washed with brine (50 mL×3), dried with Na₂SO₄, filtered and concentrated. The residue was purified by column chromatography (PE/EA=10/1) to afford 5-bromo-2-fluoro-4-methyl-N-(3-(trifluoromethyl)phenyl)benzamide (526 mg, 70% yield)

### Step 2: Synthesis of 2-fluoro-4-methyl-5-(4,4,5,5-tetramethyl-1,3,2-dioxaborolan-2-yl)-N-(3-(trifluoromethyl)phenyl)benzamide

A mixture of 5-bromo-2-fluoro-4-methyl-N-(3-(trifluoromethyl)phenyl)benzamide (376 mg, 1 mmol) in dioxane (5 mL) was added 4,4,4′,4′,5,5,5′,5′-octamethyl-2,2′-bi(1,3,2-dioxaborolane) (280 mg, 1.2 mmol), KOAc (650 mg, 6.6 mmol), Pd(dppf)Cl₂ (160 mg, 0.22 mmol). The mixture was degassed and charged with Ar for 3 times, stirred at 100° C. for 3 h. The reaction mixture was concentrated under vacuum and H₂O (50 mL) was added. The mixture was extracted with EA (100 mL×3). The combined organic phases were washed with brine (100 mL), dried over Na2SO4, filtered and concentrated. The residue was purified by column chromatography (PE/EA=10/1) to afford 2-fluoro-4-methyl-5-(4,4,5,5-tetramethyl-1,3,2-dioxaborolan-2-yl)-N-(3-(trifluoromethyl)phenyl)benzamide (300 mg, 71% yield). LCMS (M+H+) m/z: 424

### Step 3: Synthesis of 2-fluoro-4-methyl-5-(2-(methylamino)-8,9-dihydroimidazo[1′,2′:1,6]pyrido[2,3-d]pyrimidin-6-yl)-N-(3-(trifluoromethyl)phenyl)benzamide

A mixture of 6-bromo-N-methyl-8,9-dihydroimidazo[1′,2′:1,6]pyrido[2,3-d]pyrimidin-2-amine (100 mg, 0.36 mmol), 2-fluoro-4-methyl-5-(4,4,5,5-tetramethyl-1,3,2-dioxaborolan-2-yl)-N-(3-(trifluoromethyl)phenyl)benzamide (168 mg, 0.39 mmol), Cs₂CO₃ (235 mg, 0.72 mmol) and Pd(dppf)Cl₂ (58 mg, 0.07 mmol) in dioxane (15.0 mL) and water (3 mL) was degassed and charged and charged with N₂ three times and stirred at 100° C. for 16 h. The reaction mixture was cooled to r.t., diluted with water (20 mL) and extracted with EA (50 mL×3). The combined organic phase was washed with brine (100 mL), dried with Na₂SO₄, filtered and concentrated. The residue was purified by column chromatography to afford 2-fluoro-4-methyl-5-(2-(methylamino)-8,9-dihydroimidazo[1′,2′:1,6]pyrido[2,3-d]pyrimidin-6-yl)-N-(3-(trifluoromethyl)phenyl)benzamide (60 mg, 60% yield) as a white solid. ¹H NMR (400 MHz, DMSO-d₆): δ 10.66 (s, 1H), 8.22-8.17 (m, 2H), 7.93 (d, J=8.0 Hz, 1H), 7.58 (t, J=8.0 Hz, 1H), 7.51 (d, J=7.6 Hz, 1H), 7.44 (d, J=7.6 Hz, 2H), 7.26 (d, J=7.6 Hz, 1H), 7.17 (s, 1H), 4.02-3.87 (m, 4H), 2.82 (s, 3H), 2.29 (s, 3H). LCMS (M+H⁺) m/z: 497.0.

### Example 280: Preparation of 2-fluoro-4-methyl-3-(2-(methylamino)-8,9-dihydroimidazo[1′,2′:1,6]pyrido[2,3-d]pyrimidin-6-yl)-N-(3-(trifluoromethyl)phenyl)benzamide (Compound 280)

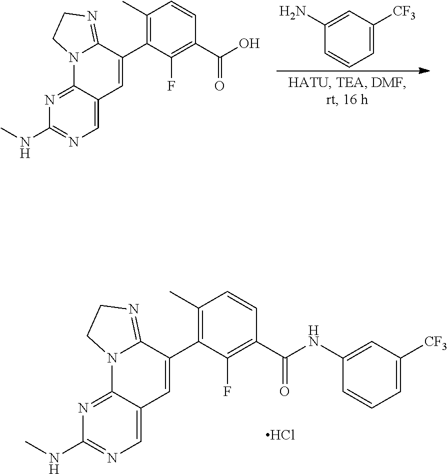 [SMILES: CNc1ncc2c(n1)N1CCN=C1C(c1c(C)ccc(C(=O)O)c1F)=C2 ; Nc1cccc(C(F)(F)F)c1 ; CNc1ncc2c(n1)N1CCN=C1C(c1c(C)ccc(C(=O)Nc3cccc(C(F)(F)F)c3)c1F)=C2 ; Cl]

### Step 1: Synthesis of 2-fluoro-4-methyl-3-(2-(methylamino)-8,9-dihydroimidazo[1′,2′:1,6]pyrido[2,3-d]pyrimidin-6-yl)-N-(3-(trifluoromethyl)phenyl)benzamide

A mixture of 2-fluoro-4-methyl-3-(2-(methylamino)-8,9-dihydroimidazo[1′,2′:1,6]pyrido[2,3-d]pyrimidin-6-yl)benzoic acid (30 mg, 0.084 mmol, 1.0 eq) and 3-(trifluoromethyl)aniline (16 mg, 0.10 mmol, and 1.2 eq) in DMF (2 mL) was added DIEA (0.1 mL) and HATU (64 mg, 0.117 mmol, 2.0 eq). The mixture was stirred at 20° C. overnight. LCMS showed the reaction was OK. The crude was diluted with H₂O (30 mL) and extracted with EA (30 mL) twice. The combined extracts were washed with brine (20 mL). Concentration and purification by prep-HPLC (0.1% HCl) afforded 2-fluoro-4-methyl-3-(2-(methylamino)-8,9-dihydroimidazo[1′,2′:1,6]pyrido[2,3-d]pyrimidin-6-yl)-N-(3-(trifluoromethyl)phenyl)benzamide (4.2 mg, 10.1% yield) as a white solid. ¹H NMR (400 MHz, DMSO-d₆): δ 10.83 (d, J=4.4 Hz, 1H), 10.22-10.21 (m, 1H), 8.98-8.88 (m, 1H), 8.60 (d, J=4.8 Hz, 1H), 8.22 (s, 2H), 7.98 (d, J=8.4 Hz, 1H), 7.78-7.74 (m, 1H), 7.63-7.59 (m, 1H), 7.48 (d, J=8.0 Hz, 1H), 7.39 (d, J=8.0 Hz, 1H), 4.69-4.62 (m, 2H), 4.09-4.05 (m, 2H), 2.98 (s, 3H), 2.29 (s, 3H). LCMS (M+H⁺) m/z: 497.1.

### Example 281: Preparation of N-(3-chloro-4-(2-(methylamino)-9,10-dihydro-8H-pyrido[1,6-a:2,3-d′]dipyrimidin-6-yl)pyridin-2-yl)furan-3-sulfonamide (Compound 281)

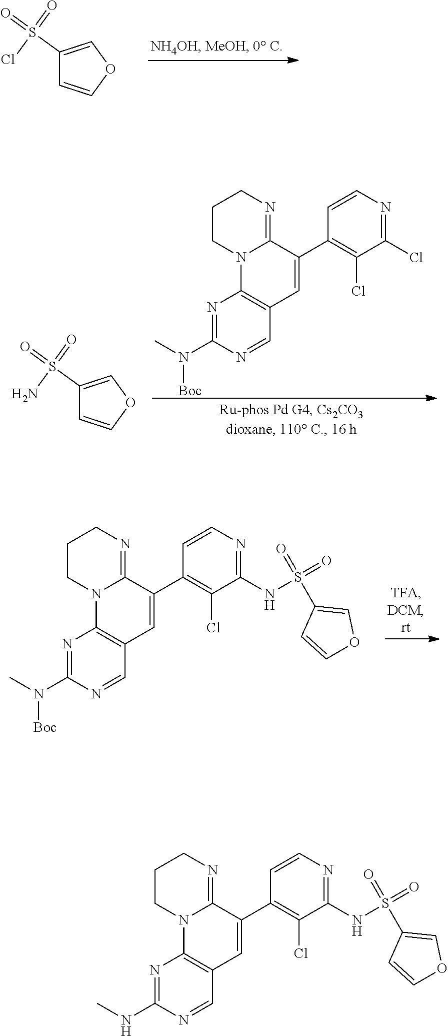 [SMILES: O=S(=O)(Cl)c1ccoc1 ; NS(=O)(=O)c1ccoc1 ; CN(C(=O)OC(C)(C)C)c1ncc2c(n1)N1CCCN=C1C(c1ccnc(Cl)c1Cl)=C2 ; CN(C(=O)OC(C)(C)C)c1ncc2c(n1)N1CCCN=C1C(c1ccnc(NS(=O)(=O)c3ccoc3)c1Cl)=C2 ; CNc1ncc2c(n1)N1CCCN=C1C(c1ccnc(NS(=O)(=O)c3ccoc3)c1Cl)=C2]

The preparation of tert-butyl (6-(2,3-dichloropyridin-4-yl)-9,10-dihydro-8H-pyrido[1,6-a:2,3-d′]dipyrimidin-2-yl)(methyl)carbamate was described in Example 74

### Step 1: Synthesis of furan-3-sulfonamide

To a solution of furan-3-sulfonyl chloride (200 mg, 1.2 mmol) in MeOH (3 mL) was added NH₄OH (3 mL). The mixture was stirred at 0° C. for 16 h. The pH was adjusted to 7 by 2N HCl. The mixture was extracted with EtOAc (2×10 mL) and the combined organic layers were washed with brine (10 mL), dried over anhydrous Na2SO4, filtered, concentrated in vacuum to give crude product furan-3-sulfonamide (120 mg, 46.5% yield) as white solid. LCMS (M+H+) m/z: 148.1

### Step 2: Synthesis of tert-butyl (6-(3-chloro-2-(furan-3-sulfonamido)pyridin-4-yl)-9,10-dihydro-8H-pyrido[1,6-a:2,3-d′]dipyrimidin-2-yl)(methyl)carbamate

To a solution of tert-butyl (6-(2,3-dichloropyridin-4-yl)-9,10-dihydro-8H-pyrido[1,6-a:2,3-d′]dipyrimidin-2-yl)(methyl)carbamate (120 mg, 0.26 mmol) in dioxane (5 mL) was added furan-3-sulfonamide (120 mg, 0.82 mmol), RuPhos Pd G4 (14 mg, 0.016 mmol) and Cs2CO3 (310 mg, 1.02 mmol). The mixture was stirred at 100° C. for 16 h. The mixture was extracted with EtOAc (2×10 mL) and the combined organic layers were washed with brine (10 mL), dried over anhydrous Na2SO4, filtered, concentrated in vacuum to give crude product tert-butyl (6-(3-chloro-2-(furan-3-sulfonamido)pyridin-4-yl)-9,10-dihydro-8H-pyrido[1,6-a:2,3-d′]dipyrimidin-2-yl)(methyl)carbamate (60 mg, crude) as yellow oil. LCMS (M+H+) m/z: 571.9

### Step 3: Synthesis of N-(3-chloro-4-(2-(methylamino)-9,10-dihydro-8H-pyrido[1,6-a:2,3-d′]dipyrimidin-6-yl)pyridin-2-yl)furan-3-sulfonamide

To a solution of tert-butyl (6-(3-chloro-2-(furan-3-sulfonamido)pyridin-4-yl)-9,10-dihydro-8H-pyrido[1,6-a:2,3-d′]dipyrimidin-2-yl)(methyl)carbamate (60 mg, crude) in DCM (3 mL) was added TFA (1 mL). The mixture was stirred at rt for 2 h. Concentration gave the crude product, which was purified by prep-HPLC (NH4HCO3) and prep-HPLC (0.1% FA) to afford N-(3-chloro-4-(2-(methylamino)-9,10-dihydro-8H-pyrido[1,6-a:2,3-d′]dipyrimidin-6-yl)pyridin-2-yl)furan-3-sulfonamide (2 mg, 4.8% yield) as yellow solid. ¹HNMR (400 MHz, CD₃OD): δ 8.64 (s, 1H), 7.98 (d, J=4.8 Hz, 1H), 7.93 (s, 1H), 7.75 (s, 1H), 7.37-7.36 (m, 1H), 6.73 (d, J=1.2 Hz, 1H), 6.56 (d, J=4.8 Hz, 1H), 4.68-4.41 (m, 2H), 3.46-3.41 (m, 2H), 2.98 (s, 3H), 2.16-2.14 (m, 2H). LCMS (M+H⁺) m/z: 472.0.

### Example 282: Preparation of N-(2-methoxy-5-(2-(methylamino)-8,9-dihydroimidazo[1′,2′:1,6]pyrido[2,3-d]pyrimidin-6-yl)pyridin-3-yl)methanesulfonamide (Compound 282)

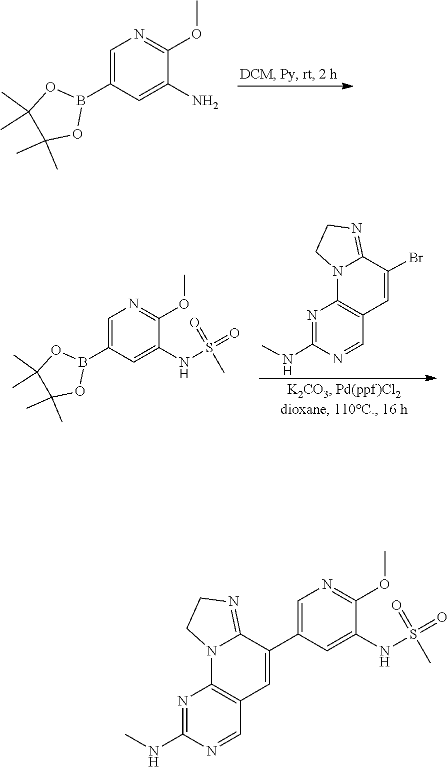

### Step 1: Synthesis of N-(2-methoxy-5-(4,4,5,5-tetramethyl-1,3,2-dioxaborolan-2-yl)pyridin-3-yl)methanesulfonamide

The mixture of 2-methoxy-5-(4,4,5,5-tetramethyl-1,3,2-dioxaborolan-2-yl)pyridin-3-amine (600 mg, 2.4 mmol, 1.0 eq) and MsCl (0.5 mL) in Pyridine (5 mL) was stirred at r.t. for 0.5 h, The mixture was concentrated. Water (30 mL) was added and reaction mixture was extracted with EA (50 mL) twice. The combined organic phases were washed with brine (50 mL), dried over Na₂SO₄ and concentrated in vacuum to afford N-(2-methoxy-5-(4,4,5,5-tetramethyl-1,3,2-dioxaborolan-2-yl)pyridin-3-yl)methanesulfonamide (600 mg) as a yellow solid. LCMS (M+H⁺) m/z: 329.2

### Step 2: Synthesis of N-(2-methoxy-5-(2-(methylamino)-8,9-dihydroimidazo[1′,2′:1,6]pyrido[2,3-d]pyrimidin-6-yl)pyridin-3-yl)methanesulfonamide

The mixture of N-(2-methoxy-5-(4,4,5,5-tetramethyl-1,3,2-dioxaborolan-2-yl)pyridin-3-yl)methanesulfonamide (117 mg, 0.36 mmol, 1.0 eq), 6-bromo-N-methyl-8,9-dihydroimidazo[1′,2′:1,6]pyrido[2,3-d]pyrimidin-2-amine (100 mg, 0.36 mmol, 1.0 eq), K₂CO₃ (150 mg, 1.07 mmol, 3.0 eq) and Pd (dppf)Cl₂ (26 mg, 0.036 mmol) in dioxane (20 mL) and H₂O (1.5 mL) was stirred at 100° C. for 3 h. The mixture was purified by Prep-HPLC (0.1% FA and 0.1% NH₄HCO₃) to afford N-(2-methoxy-5-(2-(methylamino)-8,9-dihydroimidazo[1′,2′:1,6]pyrido[2,3-d]pyrimidin-6-yl)pyridin-3-yl)methanesulfonamide (2.1 mg) as yellow solid. ¹H NMR (400 MHz, DMSO-d₆): δ 10.07 (br s, 1H), 9.44-9.41 (m, 1H), 8.70 (s, 1H), 8.25-8.21 (m, 1H), 8.02-7.84 (m, 2H), 4.49-4.43 (m, 2H), 4.05-3.98 (m, 5H), 3.09 (s, 3H), 2.93-2.9-87 (m, 3H). LCMS (M+H⁺) m/z: 402.2.

### Example 283: Preparation of N-(4-methyl-3-(2-(methylamino)-8,9-dihydroimidazo[1′,2′:1,6]pyrido[2,3-d]pyrimidin-6-yl)phenyl)-3-(trifluoromethyl)benzenesulfonamide (Compound 283)

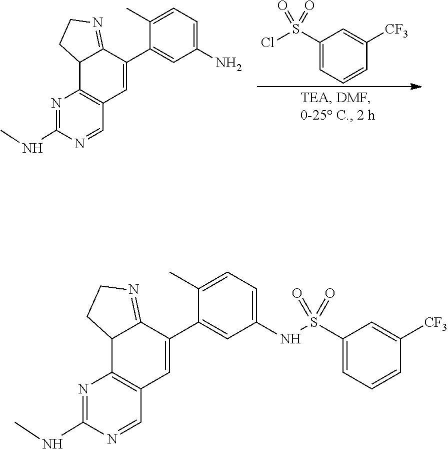 [SMILES: CNc1ncc2c(n1)C1CCN=C1C(c1cc(N)ccc1C)=C2 ; O=S(=O)(Cl)c1cccc(C(F)(F)F)c1 ; CNc1ncc2c(n1)C1CCN=C1C(c1cc(NS(=O)(=O)c3cccc(C(F)(F)F)c3)ccc1C)=C2]

### Step 1: Synthesis of N-(4-methyl-3-(2-(methylamino)-8,9-dihydroimidazo[1′,2′:1,6]pyrido[2,3-d]pyrimidin-6-yl)phenyl)-3-(trifluoromethyl)benzenesulfonamide

To a mixture of 6-(5-amino-2-methylphenyl)-N-methyl-8,9-dihydroimidazo[1′,2′:1,6]pyrido[2,3-d]pyrimidin-2-amine (25 mg, 0.09 mmol), TEA (27 mg, 0.27 mmol) in DMF (2 mL) was added 3-(trifluoromethyl)benzenesulfonyl chloride (17 mg, 0.08 mmol) at 0° C. under N₂. The mixture was stirred at r.t. for 2 h. The reaction mixture was concentrated and purified by Prep-HPLC (0.1% NH₃·H₂O) to afford N-(4-methyl-3-(2-(methylamino)-8,9-dihydroimidazo[1′,2′:1,6]pyrido[2,3-d]pyrimidin-6-yl)phenyl)-3-(trifluoromethyl)benzenesulfonamide (4.9 mg, 10% yield) as yellow solid. ¹H NMR (400 MHz, DMSO-d₆): δ 10.31 (br, 1H), 8.22 (br, 1H), 8.03-8.01 (m, 3H), 7.82 (t, J=8.0 Hz, 1H), 7.62-7.34 (m, 1H), 7.11 (d, J=8.0 Hz, 1H), 6.97-6.94 (m, 3H), 4.12-3.95 (m, 2H), 3.89-3.85 (m, 2H), 2.85 (s, 3H), 2.10 (s, 3H). LCMS (M+H⁺) m/z: 515.2.

### Example 284: Preparation of N-(4-methyl-3-(2-(methylamino)-8,9-dihydroimidazo[1′,2′:1,6]pyrido[2,3-d]pyrimidin-6-yl)phenyl)-2-(trifluoromethyl)benzenesulfonamide (Compound 284)

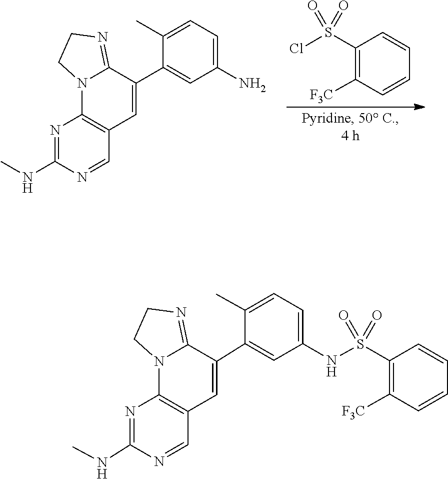 [SMILES: CNc1ncc2c(n1)N1CCN=C1C(c1cc(N)ccc1C)=C2 ; O=S(=O)(Cl)c1ccccc1C(F)(F)F ; CNc1ncc2c(n1)N1CCN=C1C(c1cc(NS(=O)(=O)c3ccccc3C(F)(F)F)ccc1C)=C2]

### Step 1: Synthesis of 2-fluoro-N-(4-methyl-3-(2-(methylamino)-8,9-dihydroimidazo[1′,2′:1,6]pyrido[2,3-d]pyrimidin-6-yl)phenyl)benzenesulfonamide

To a mixture of 6-(5-amino-2-methylphenyl)-N-methyl-8,9-dihydroimidazo[1′,2′:1,6]pyrido[2,3-d]pyrimidin-2-amine (60 mg, 0.196 mmol) in pyridine (10 mL) was added 2-(trifluoromethyl)benzenesulfonyl chloride (143 mg, 0.588 mmol) at r.t., the mixture was stirred at 55° C. for 2 h. The reaction mixture was concentrated in vacuum and purified by column chromatography on silica gel (DCM/MeOH=10/1) to give crude product, which was purified by Prep-HPLC (0.1% NH₃·H₂O) to afford 2-fluoro-N-(4-methyl-3-(2-(methylamino)-8,9-dihydroimidazo[1′,2′:1,6]pyrido[2,3-d]pyrimidin-6-yl)phenyl)benzenesulfonamide (43.6 mg, 43% yield) as yellow solid. ¹H NMR (400 MHz, DMSO-d₆): δ 10.29 (s, 1H), 8.17-8.14 (m, 1H), 8.01-7.96 (m, 3H), 7.77 (t, J=8.0 Hz, 1H), 7.39 (s, 1H), 7.01 (d, J=8.0 Hz, 1H), 6.88-6.87 (m, 3H), 4.01-3.96 (m, 2H), 3.87 (t, J=8.8 Hz, 2H), 2.83 (s, 3H), 2.08 (s, 3H). LCMS (M+H⁺) m/z: 515.6.

### Example 285: Preparation of N-(2-fluoro-4-methyl-5-(2-(methylamino)-8,9-dihydroimidazo[1′,2′:1,6]pyrido[2,3-d]pyrimidin-6-yl)phenyl)-3-(trifluoromethyl)benzenesulfonamide (Compound 285)

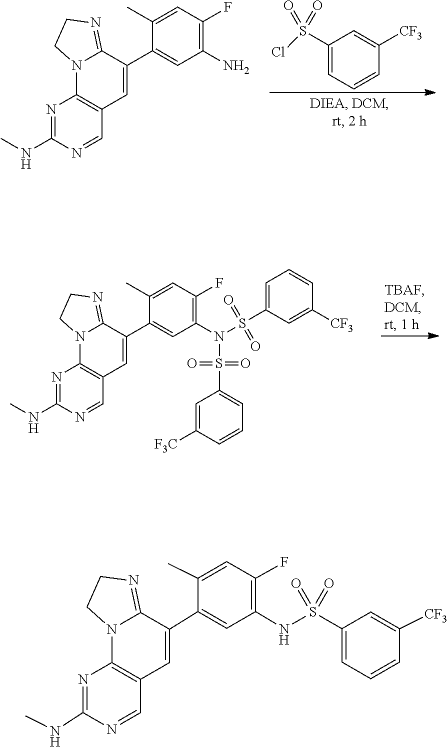 [SMILES: CNc1ncc2c(n1)N1CCN=C1C(c1cc(N)c(F)cc1C)=C2 ; O=S(=O)(Cl)c1cccc(C(F)(F)F)c1 ; CNc1ncc2c(n1)N1CCN=C1C(c1cc(N(S(=O)(=O)c3cccc(C(F)(F)F)c3)S(=O)(=O)c3cccc(C(F)(F)F)c3)c(F)cc1C)=C2 ; CNc1ncc2c(n1)N1CCN=C1C(c1cc(NS(=O)(=O)c3cccc(C(F)(F)F)c3)c(F)cc1C)=C2]

### Step 1: Synthesis of N-(2-fluoro-4-methyl-5-(2-(methylamino)-8,9-dihydroimidazo[1′,2′:1,6]pyrido[2,3-d]pyrimidin-6-yl)phenyl)-3-(trifluoromethyl)-N-((3-(trifluoromethyl)phenyl)sulfonyl)benzenesulfonamide

To a solution of 6-(5-amino-4-fluoro-2-methylphenyl)-N-methyl-8,9-dihydroimidazo[1′,2′:1,6]pyrido[2,3-d]pyrimidin-2-amine (50 mg, 0.20 mmol), DIEA (53 mg, 0.41 mmol) in DCM (5.0 mL) was added 3-(trifluoromethyl)benzenesulfonyl chloride (198 mg, 0.61 mmol) at r.t. under N₂. The resulting mixture was stirred at 80° C. for 16 h under N₂. The reaction mixture was concentrated in vacuum and purified by column chromatography on silica gel (DCM/MeOH=10/1) to afford N-(2-fluoro-4-methyl-5-(2-(methylamino)-8,9-dihydroimidazo[1′,2′:1,6]pyrido[2,3-d]pyrimidin-6-yl)phenyl)-3-(trifluoromethyl)-N-((3-(trifluoromethyl)phenyl)sulfonyl)benzenesulfonamide (40 mg, 27% yield) as brown solid. LCMS (M+H⁺) m/z: 740.8.

### Step 2: Synthesis of N-(2-fluoro-4-methyl-5-(2-(methylamino)-8,9-dihydroimidazo[1′,2′:1,6]pyrido[2,3-d]pyrimidin-6-yl)phenyl)-3-(trifluoromethyl)benzenesulfonamide

To a solution of N-(2-fluoro-4-methyl-5-(2-(methylamino)-8,9-dihydroimidazo[1′,2′:1,6]pyrido[2,3-d]pyrimidin-6-yl)phenyl)-3-(trifluoromethyl)-N-((3-(trifluoromethyl)phenyl)sulfonyl)benzenesulfonamide (40 mg, 0.054 mmol) in DCM (5.0 mL) was added TBAF (12 mg, 0.054 mmol) at r.t. for 0.5 h under N₂. The reaction mixture was concentrated in vacuum and purified by column chromatography on silica gel (DCM/MeOH=10/1) to give crude product, which was purified by Pre-HPLC (0.1% FA) to afford N-(2-fluoro-4-methyl-5-(2-(methylamino)-8,9-dihydroimidazo[1′,2′:1,6]pyrido[2,3-d]pyrimidin-6-yl)phenyl)-3-(trifluoromethyl)benzenesulfonamide (5.0 mg, 17.5% yield) as a yellow solid. ¹H NMR (400 MHz, DMSO-d₆): δ 10.20 (br s, 1H), 8.29-8.28 (m, 1H), 8.01-7.98 (m, 3H), 7.79-7.75 (m, 1H), 7.65-7.52 (m, 1H), 7.05-6.99 (m, 3H), 4.15-4.11 (m, 2H), 3.92-3.88 (m, 2H), 2.86 (s, 3H), 2.12 (s, 3H). LCMS (M+H⁺) m/z: 533.2.

### Example 286: Preparation of 2-chloro-N-(2-fluoro-4-methyl-5-(2-(methylamino)-8,9-dihydroimidazo[1′,2′:1,6]pyrido[2,3-d]pyrimidin-6-yl)phenyl)benzenesulfonamide (Compound 286)

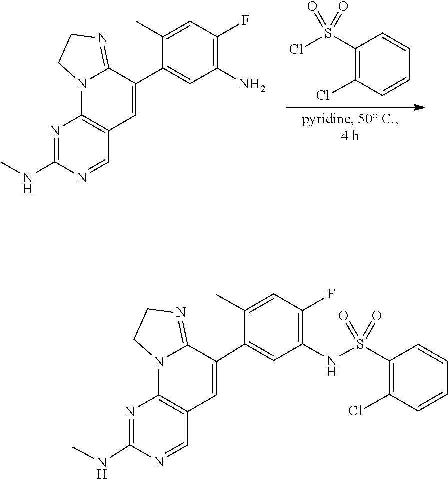 [SMILES: CNc1ncc2c(n1)N1CCN=C1C(c1cc(N)c(F)cc1C)=C2 ; O=S(=O)(Cl)c1ccccc1Cl ; CNc1ncc2c(n1)N1CCN=C1C(c1cc(NS(=O)(=O)c3ccccc3Cl)c(F)cc1C)=C2]

### Step 1: Synthesis of 2-chloro-N-(2-fluoro-4-methyl-5-(2-(methylamino)-8,9-dihydroimidazo[1′,2′:1,6]pyrido[2,3-d]pyrimidin-6-yl)phenyl)benzenesulfonamide

A mixture of 6-(5-amino-4-fluoro-2-methylphenyl)-N-methyl-8,9-dihydroimidazo[1′,2′:1,6]pyrido[2,3-d]pyrimidin-2-amine (100 mg, 0.31 mmol) in pyridine (10 mL) was added 2-chlorobenzenesulfonyl chloride (200 mg, 0.93 mmol) at r.t., the mixture was stirred at 40° C. for 2 h. The reaction mixture was concentrated and purified by Prep-HPLC (0.1% NH3·H2O) to afford 2-chloro-N-(2-fluoro-4-methyl-5-(2-(methylamino)-8,9-dihydroimidazo[1′,2′:1,6]pyrido[2,3-d]pyrimidin-6-yl)phenyl)benzenesulfonamide (22.0 mg, 13% yield) as yellow solid. ¹H NMR (400 MHz, DMSO-d₆): δ 8.16-8.15 (m, 1H), 7.88 (dd, J=7.6, 1.6 Hz, 1H), 7.49-7.42 (m, 2H), 7.40-7.33 (m, 2H), 7.10-6.94 (m, 2H), 6.83-6.80 (m, 2H), 4.01-3.94 (m, 2H), 3.86 (t, J=8.4 Hz, 2H), 2.83 (d, J=4.4 Hz, 3H), 2.04 (s, 3H). LCMS (M+H⁺) m/z: 499.1.

### Example 287: Preparation of 2-chloro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (Compound 287)

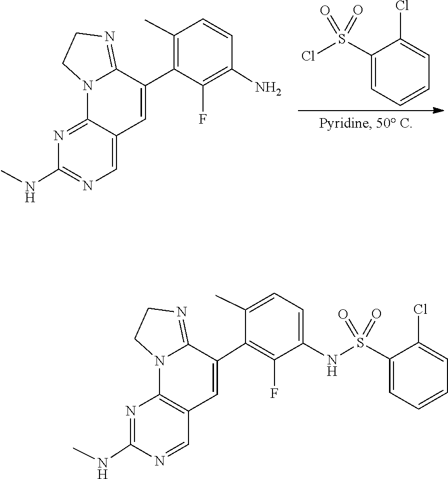 [SMILES: CNc1ncc2c(n1)N1CCN=C1C(c1c(C)ccc(N)c1F)=C2 ; O=S(=O)(Cl)c1ccccc1Cl ; CNc1ncc2c(n1)N1CCN=C1C(c1c(C)ccc(NS(=O)(=O)c3ccccc3Cl)c1F)=C2]

### Step 1: Synthesis of 2-chloro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide

To a solution of 6-(3-amino-2-fluoro-6-methylphenyl)-N-methyl-8,9-dihydroimidazo[1′,2′:1,6]pyrido[2,3-d]pyrimidin-2-amine (40 mg, 0.123 mmol) in pyridine (3 mL) was added 2-chlorobenzenesulfonyl chloride (52 mg, 0.247 mmol). The reaction was stirred at 50° C. for 2 h. The solvent was removed and then purified on prep-HPLC (0.1% TFA) to afford 2-chloro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide as (4.1 mg, 10% yield) a white solid. ¹H NMR (400 MHz, CD₃OD): δ 8.77 (s, 1H), 8.05 (d, J=7.6 Hz, 1H), 7.99 (s, 1H), 7.60 (d, J=3.6 Hz, 2H), 7.47-7.39 (m, 2H), 7.13 (d, J=8.4 Hz, 1H), 4.78 (t, J=10.0 Hz, 2H), 4.12 (t, J=10.0 Hz, 2H), 3.09 (s, 3H), 2.19 (s, 3H). LCMS (M+H⁺) m/z: 499.1.

### Example 288: Preparation of N-(2-fluoro-4-methyl-3-(2-(methylamino)-8,9-dihydroimidazo[1′,2′:1,6]pyrido[2,3-d]pyrimidin-6-yl)phenyl)propane-1-sulfonamide (Compound 288)

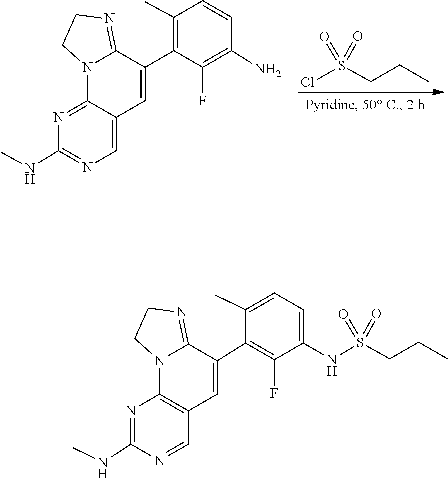

### Step 1: Synthesis of N-(2-fluoro-4-methyl-3-(2-(methylamino)-8,9-dihydroimidazo[1′,2′:1,6]pyrido[2,3-d]pyrimidin-6-yl)phenyl)propane-1-sulfonamide

To a solution of 6-(3-amino-2-fluoro-6-methylphenyl)-N-methyl-8,9-dihydroimidazo[1′,2′:1,6]pyrido[2,3-d]pyrimidin-2-amine (40 mg, 0.12 mmol) in pyridine (4 mL) was added propane-1-sulfonyl chloride (20 mg, 1.1 mmol) at 20° C. The reaction mixture was stirred at 50° C. for 2 h. LCMS show the reaction was OK. The reaction mixture was concentrated in vacuum and purified by Prep-HPLC (0.1% HCl) to afford N-(2-fluoro-4-methyl-3-(2-(methylamino)-8,9-dihydroimidazo[1′,2′:1,6]pyrido[2,3-d]pyrimidin-6-yl)phenyl)propane-1-sulfonamide (4.4 mg, 8%) as a yellow solid. ¹H NMR (400 MHz, CD₃OD): δ 8.71 (s, 1H), 8.02 (s, 1H), 7.45 (t, J=8.4 Hz, 1H), 7.13 (d, J=8.4 Hz, 1H), 4.73-4.68 (m, 2H), 4.08-4.03 (m, 2H), 3.05-2.99 (m, 5H), 2.15 (s, 3H), 1.79-1.73 (m, 2H), 0.95 (t, J=7.2 Hz, 3H). LCMS (M+H⁺) m/z: 431.2.

### Example 289: Preparation of 2-fluoro-N-(4-methyl-3-(2-(methylamino)-8,9-dihydroimidazo[1′,2′:1,6]pyrido[2,3-d]pyrimidin-6-yl)phenyl)benzenesulfonamide (Compound 289)

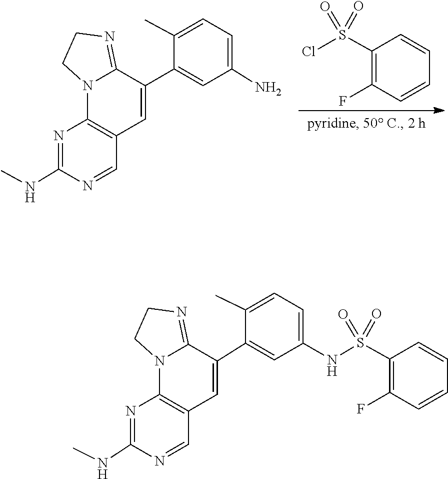 [SMILES: CNc1ncc2c(n1)N1CCN=C1C(c1cc(N)ccc1C)=C2 ; O=S(=O)(Cl)c1ccccc1F ; CNc1ncc2c(n1)N1CCN=C1C(c1cc(NS(=O)(=O)c3ccccc3F)ccc1C)=C2]

### Step 1: Synthesis of 2-fluoro-N-(4-methyl-3-(2-(methylamino)-8,9-dihydroimidazo[1′,2′:1,6]pyrido[2,3-d]pyrimidin-6-yl)phenyl)benzenesulfonamide

To a mixture of 6-(5-amino-2-methylphenyl)-N-methyl-8,9-dihydroimidazo[1′,2′:1,6]pyrido[2,3-d]pyrimidin-2-amine (100 mg, 0.33 mmol) in pyridine (10 mL) was added 2-fluorobenzenesulfonyl chloride (190 mg, 0.98 mmol) at r.t., the mixture was stirred at r.t. for 2 h. The reaction mixture was concentrated and purified by Prep-HPLC (0.1% NH₃·H₂O) to afford 2-fluoro-N-(4-methyl-3-(2-(methylamino)-8,9-dihydroimidazo[1′,2′:1,6]pyrido[2,3-d]pyrimidin-6-yl)phenyl)benzenesulfonamide (55.1 mg, 36% yield) as yellow solid. ¹H NMR (400 MHz, DMSO-d₆): δ 8.42-8.41 (m, 1H), 8.14 (s, 1H), 7.85-7.81 (m, 1H), 7.73-7.67 (m, 2H), 7.45-7.35 (m, 3H), 7.12-7.11 (m, 1H), 7.03-7.01 (m, 2H), 4.25-4.08 (m, 2H), 3.90 (t, J=8.8 Hz, 2H), 2.87 (s, 3H), 2.08 (s, 3H). LCMS (M+H⁺) m/z: 465.2.

### Example 290: Preparation of 2,6-difluoro-N-(4-methyl-3-(2-(methylamino)-8,9-dihydroimidazo[1′,2′:1,6]pyrido[2,3-d]pyrimidin-6-yl)phenyl)benzenesulfonamide (Compound 290)

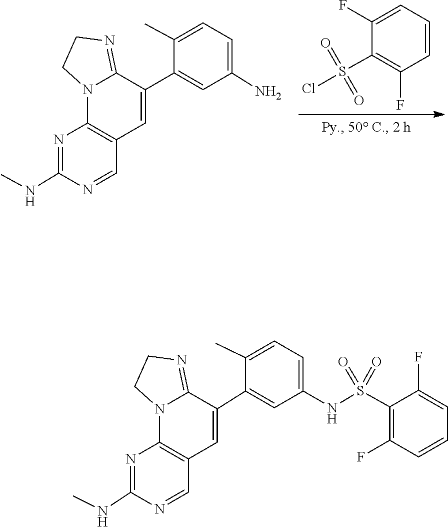 [SMILES: CNc1ncc2c(n1)N1CCN=C1C(c1cc(N)ccc1C)=C2 ; O=S(=O)(Cl)c1c(F)cccc1F ; CNc1ncc2c(n1)N1CCN=C1C(c1cc(NS(=O)(=O)c3c(F)cccc3F)ccc1C)=C2]

### Step 1: Synthesis of 2,6-Difluoro-N-(4-methyl-3-(2-(methylamino)-8,9-dihydroimidazo[1′,2′:1,6]pyrido[2,3-d]pyrimidin-6-yl)phenyl)benzenesulfonamide

To a mixture of 6-(5-amino-2-methylphenyl)-N-methyl-8,9-dihydroimidazo[1′,2′:1,6]pyrido[2,3-d]pyrimidin-2-amine (50 mg, 0.16 mmol) in Pyridine (1.5 mL) was added 2,6-difluorobenzenesulfonyl chloride (104 mg, 0.49 mmol). The mixture was stirred at 50° C. for 2 hours. The reaction mixture was concentrated and purified by column chromatography on silica gel (DCM:MeOH=20:1) to give the crude product, which was further purified by Prep-HPLC (0.1% NH₃H₂O) to afford 2,6-difluoro-N-(4-methyl-3-(2-(methylamino)-8,9-dihydroimidazo[1′,2′:1,6]pyrido[2,3-d]pyrimidin-6-yl)phenyl)benzenesulfonamide (27.6 mg, 25% yield) as a yellow solid. ¹H NMR (400 MHz, DMSO-d₆): δ 10.75 (s, 1H), 8.22-8.19 (m, 1H), 7.70-7.67 (m, 1H), 7.44-7.42 (m, 1H), 7.26 (t, J=8.8 Hz, 2H), 7.08 (d, J=8.4 Hz, 1H), 6.99 (d, J=8.4 Hz, 1H), 6.94 (s, 2H), 4.01-3.96 (m, 2H), 3.86 (t, J=8.8 Hz, 2H), 2.84 (d, J=3.6 Hz, 3H), 2.10 (s, 3H). LCMS (M+H⁺) m/z: 483.2.

### Example 291: Preparation of 2,5-difluoro-N-(4-methyl-3-(2-(methylamino)-8,9-dihydroimidazo[1′,2′:1,6]pyrido[2,3-d]pyrimidin-6-yl)phenyl)benzenesulfonamide (Compound 291)

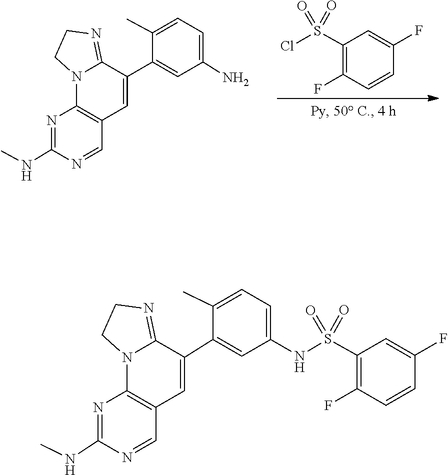 [SMILES: CNc1ncc2c(n1)N1CCN=C1C(c1cc(N)ccc1C)=C2 ; O=S(=O)(Cl)c1cc(F)ccc1F ; CNc1ncc2c(n1)N1CCN=C1C(c1cc(NS(=O)(=O)c3cc(F)ccc3F)ccc1C)=C2]

### Step 1: Synthesis of 2,5-difluoro-N-(4-methyl-3-(2-(methylamino)-8,9-dihydroimidazo[1′,2′:1,6]pyrido[2,3-d]pyrimidin-6-yl)phenyl)benzenesulfonamide

To a mixture of 6-(5-amino-2-methylphenyl)-N-methyl-8,9-dihydroimidazo[1′,2′:1,6]pyrido[2,3-d]pyrimidin-2-amine (80 mg, 0.26 mmol) in pyridine (10 mL) was added 2,5-difluorobenzenesulfonyl chloride (167 mg, 0.78 mmol) at r.t., the mixture was stirred at 40° C. for 2 h. The reaction mixture was concentrated and purified by Prep-HPLC (0.1% NH₃·H₂O) to afford 2,5-difluoro-N-(4-methyl-3-(2-(methylamino)-8,9-dihydroimidazo[1′,2′:1,6]pyrido[2,3-d]pyrimidin-6-yl)phenyl)benzenesulfonamide (27.3 mg, 21.8% yield) as yellow solid. ¹H NMR (400 MHz, DMSO-d₆): δ 10.63 (s, 1H), 8.22-8.19 (m, 1H), 7.62-7.42 (m, 4H), 7.09 (d, J=8.4 Hz, 1H), 6.99-6.96 (m, 3H), 4.07-3.96 (m, 2H), 3.87 (t, J=8.8 Hz, 2H), 2.83 (d, J=3.6 Hz, 3H), 2.10 (s, 3H). LCMS (M+H⁺) m/z: 483.7.

### Example 292: Preparation of N-(2-fluoro-4-methyl-5-(2-(methylamino)-8,9-dihydroimidazo[1′,2′:1,6]pyrido[2,3-d]pyrimidin-6-yl)phenyl)pyridine-2-sulfonamide (Compound 292)

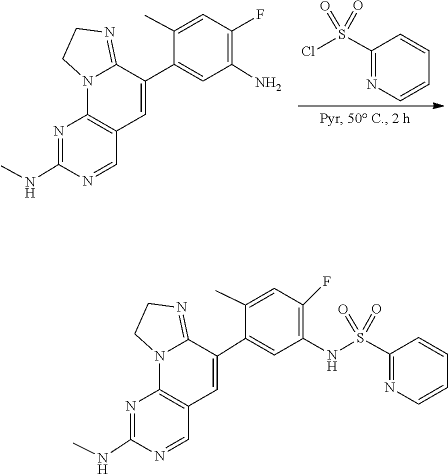 [SMILES: CNc1ncc2c(n1)N1CCN=C1C(c1cc(N)c(F)cc1C)=C2 ; O=S(=O)(Cl)c1ccccn1 ; CNc1ncc2c(n1)N1CCN=C1C(c1cc(NS(=O)(=O)c3ccccn3)c(F)cc1C)=C2]

### Step 1: Synthesis of N-(2-fluoro-4-methyl-5-(2-(methylamino)-8,9-dihydroimidazo[1′,2′:1,6]pyrido[2,3-d]pyrimidin-6-yl)phenyl)pyridine-2-sulfonamide

To a mixture of 6-(5-amino-4-fluoro-2-methylphenyl)-N-methyl-8,9-dihydroimidazo[1′,2′:1,6]pyrido[2,3-d]pyrimidin-2-amine (50 mg, 0.15 mmol) in pyridine (1.5 mL) was added pyridine-2-sulfonyl chloride (137 mg, 0.77 mmol). The mixture was stirred at 50° C. for 2 hours. The reaction mixture was concentrated and purified by column chromatography on silica gel (DCM:MeOH=20:1) to give the crude product, which was further purified by Prep-HPLC (0.1% HCOOH) to afford N-(2-fluoro-4-methyl-5-(2-(methylamino)-8,9-dihydroimidazo[1′,2′:1,6]pyrido[2,3-d]pyrimidin-6-yl)phenyl)pyridine-2-sulfonamide (33.8 mg, 47% yield) as a yellow solid. ¹H NMR (400 MHz, DMSO-d₆): δ 8.72 (d, J=4.0 Hz, 1H), 8.24 (s, 1H), 8.19 (s, 1H), 8.07-8.02 (m, 1H), 7.86 (d, J=7.6 Hz, 1H), 7.67-7.64 (m, 1H), 7.49-7.44 (m, 1H), 7.06-7.01 (m, 3H), 4.06-3.90 (m, 2H), 3.86 (t, J=9.6 Hz, 2H), 2.85 (d, J=4.4 Hz, 3H), 2.14 (s, 3H). LCMS (M+H⁺) m/z: 466.2.

### Example 293: Preparation of N-(2-fluoro-4-methyl-5-(2-(methylamino)-8,9-dihydroimidazo[1′,2′:1,6]pyrido[2,3-d]pyrimidin-6-yl)phenyl)-1-phenylmethanesulfonamide (Compound 293)

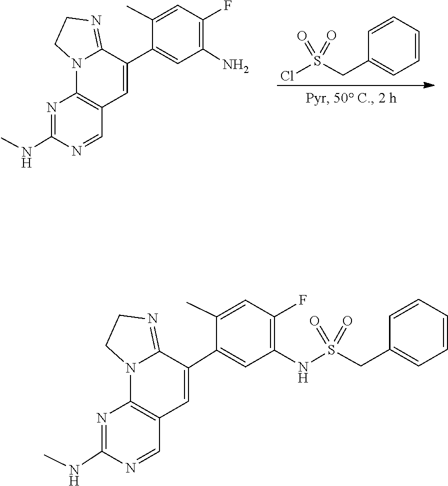 [SMILES: CNc1ncc2c(n1)N1CCN=C1C(c1cc(N)c(F)cc1C)=C2 ; O=S(=O)(Cl)Cc1ccccc1 ; CNc1ncc2c(n1)N1CCN=C1C(c1cc(NS(=O)(=O)Cc3ccccc3)c(F)cc1C)=C2]

### Step 1: Synthesis of N-(2-fluoro-4-methyl-5-(2-(methylamino)-8,9-dihydroimidazo[1′,2′:1,6]pyrido[2,3-d]pyrimidin-6-yl)phenyl)-1-phenylmethanesulfonamide

To a mixture of 6-(5-amino-4-fluoro-2-methylphenyl)-N-methyl-8,9-dihydroimidazo[1′,2′:1,6]pyrido[2,3-d]pyrimidin-2-amine (50 mg, 0.15 mmol) in pyridine (1.5 mL) was added phenylmethanesulfonyl chloride (88 mg, 0.46 mmol). The mixture was stirred at 50° C. for 2 hours. The reaction mixture was concentrated and purified by column chromatography on silica gel (DCM:MeOH=20:1) to give the crude product, which was further purified by Prep-HPLC (0.1% HCOOH) to afford N-(2-fluoro-4-methyl-5-(2-(methylamino)-8,9-dihydroimidazo[1′,2′:1,6]pyrido[2,3-d]pyrimidin-6-yl)phenyl)-1-phenylmethanesulfonamide (22.4 mg, 30% yield) as a yellow solid. ¹H NMR (400 MHz, DMSO-d₆): δ 8.41 (s, 1H), 8.14 (s, 1H), 7.36-7.33 (m, 5H), 7.29-7.18 (m, 3H), 7.08 (d, J=8.4 Hz, 1H), 4.47 (s, 2H), 4.24-4.07 (m, 2H), 3.93 (t, J=8.8 Hz, 2H), 2.88 (s, 3H), 2.19 (s, 3H). LCMS (M+H⁺) m/z: 479.2.

### Example 294: Preparation of 2-fluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (Compound 294)

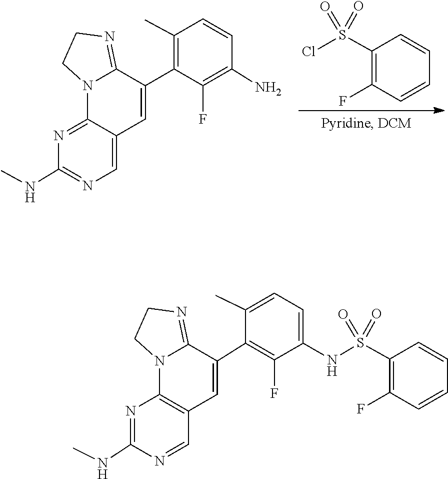 [SMILES: CNc1ncc2c(n1)N1CCN=C1C(c1c(C)ccc(N)c1F)=C2 ; O=S(=O)(Cl)c1ccccc1F ; CNc1ncc2c(n1)N1CCN=C1C(c1c(C)ccc(NS(=O)(=O)c3ccccc3F)c1F)=C2]

### Step 1: Synthesis of 2-fluoro-N-(2-fluoro-4-methyl-3-(2-(methylamino)-8,9-di hydroimidazo[1′,2′:1,6]pyrido[2,3-d]pyrimidin-6-yl)phenyl)benzenesulfonamide formate

To a solution of 6-(3-amino-2-fluoro-6-methylphenyl)-N-methyl-8,9-dihydroimidazo[1′,2′:1,6]pyrido[2,3-d]pyrimidin-2-amine (50 mg, 0.15 mmol, 1.0 eq) in DCM (5 mL) was added pyridine (0.1 mL) and 2-fluorobenzenesulfonyl chloride (58 mg, 0.3 mmol, 2.0 eq). The mixture was stirred at 20° C. under N2 for 16 h. LCMS showed the reaction was OK. Concentration in vacuum and purification by Prep-HPLC (0.1% FA) afforded 2-fluoro-N-(2-fluoro-4-methyl-3-(2-(methylamino)-8,9-di hydroimidazo[1′,2′:1,6]pyrido[2,3-d]pyrimidin-6-yl)phenyl)benzenesulfonamide formate (7.2 mg, 6.4%) as a yellow solid. ¹H NMR (400 MHz, CD₃OD): δ 8.40 (s, 1H), 7.80-7.77 (m, 1H), 7.66-7.60 (m, 1H), 7.43-7.27 (m, 4H), 7.04-7.02 (m, 1H), 4.44-4.37 (m, 2H), 4.02-3.97 (m, 2H), 3.01 (s, 3H), 2.16 (s, 3H). LCMS (M+H⁺) m/z: 483.0.

### Example 295: Preparation of 2,5-difluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (Compound 295)

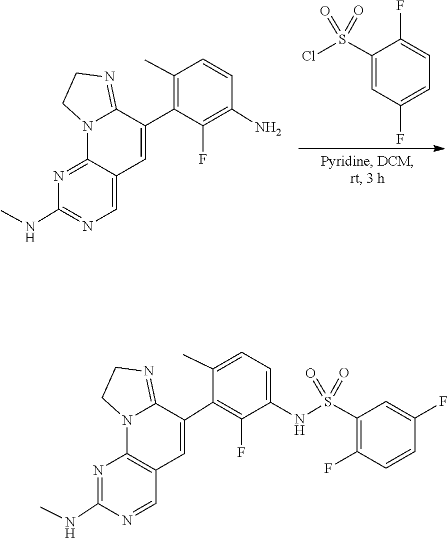 [SMILES: CNc1ncc2c(n1)N1CCN=C1C(c1c(C)ccc(N)c1F)=C2 ; O=S(=O)(Cl)c1cc(F)ccc1F ; CNc1ncc2c(n1)N1CCN=C1C(c1c(C)ccc(NS(=O)(=O)c3cc(F)ccc3F)c1F)=C2]

### Step 1: Synthesis of 2,5-difluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide

To a solution of 6-(3-amino-2-fluoro-6-methylphenyl)-N-methyl-8,9-dihydroimidazo[1′,2′:1,6]pyrido[2,3-d]pyrimidin-2-amine (20 mg 0.077 mmol) in pyridine (2 mL) was added 2,5-difluorobenzenesulfonyl chloride (33 mg, 0.154 mmol). The reaction mixture was stirred at rt for 3 h. The resulting mixture was purified by prep-HPLC (0.1% TFA) to afford 2,5-difluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (10.8 mg, 35.1% yield) as a white solid. ¹H NMR (400 MHz, DMSO-d₆): δ 10.69 (s, 1H), 9.85 (s, 1H), 8.84 (s, 1H), 8.58-8.56 (m, 1H), 8.06 (s, 1H), 7.62-7.53 (m, 3H), 7.28 (t, J=8.0 Hz, 1H), 7.18 (d, J=8.4 Hz, 1H), 4.63-4.60 (m, 2H), 4.00-3.98 (m, 2H), 2.96 (d, J=4.8 Hz, 3H), 2.14 (s, 3H). LCMS (M+H⁺) m/z: 501.0.

### Example 296: Preparation of 2,6-dichloro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (Compound 296)

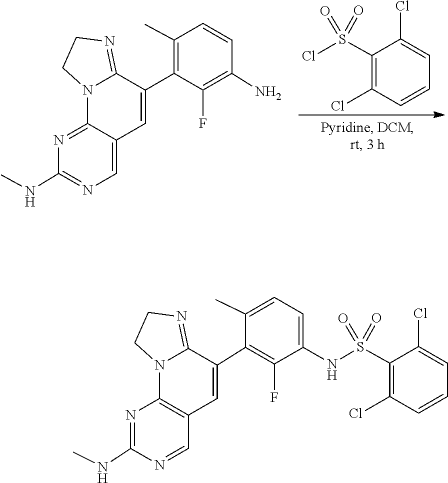 [SMILES: CNc1ncc2c(n1)N1CCN=C1C(c1c(C)ccc(N)c1F)=C2 ; O=S(=O)(Cl)c1c(Cl)cccc1Cl ; CNc1ncc2c(n1)N1CCN=C1C(c1c(C)ccc(NS(=O)(=O)c3c(Cl)cccc3Cl)c1F)=C2]

### Step 1: Synthesis of 2,6-dichloro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide

To a mixture of 6-(3-amino-2-fluoro-6-methylphenyl)-N-methyl-8,9-dihydroimidazo[1′,2′:1,6]pyrido[2,3-d]pyrimidin-2-amine (30 mg, 0.093 mmol, 1 eq) in DCM (5 mL) was added pyridine (1 mL) and 2,6-dichlorobenzenesulfonyl chloride (46 mg, 0.186 mmol, 2 eq). The mixture was stirred at rt under N2 for 3 h. The resulting mixture was purified by prep-HPLC (0.1% NH3·H2O) to afford 2,6-dichloro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (3.8 mg, 7.9% yield) as a yellow solid. ¹H NMR (400 MHz, CD₃OD): δ 8.33 (s, 1H), 7.51-7.48 (m, 2H), 7.41-7.37 (m, 1H), 7.26 (t, J=8.0 Hz, 2H), 6.97 (d, J=8.8 Hz, 1H), 4.36-4.42 (m, 2H), 4.00-3.96 (m, 2H), 2.99 (s, 3H), 2.15 (s, 3H). LCMS (M+H⁺) m/z: 533.0.

### Example 297: Preparation of 2,6-difluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (Compound 297)

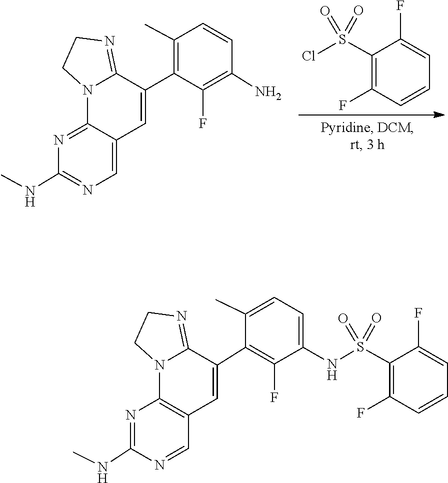 [SMILES: CNc1ncc2c(n1)N1CCN=C1C(c1c(C)ccc(N)c1F)=C2 ; O=S(=O)(Cl)c1c(F)cccc1F ; CNc1ncc2c(n1)N1CCN=C1C(c1c(C)ccc(NS(=O)(=O)c3c(F)cccc3F)c1F)=C2]

### Step 1: Synthesis of 2,6-difluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide

To a mixture of 6-(3-amino-2-fluoro-6-methylphenyl)-N-methyl-8,9-dihydroimidazo[1′,2′:1,6]pyrido[2,3-d]pyrimidin-2-amine (30 mg, 0.093 mmol, 1 eq) in DCM (5 mL) was added pyridine (1 mL) and 2,6-difluorobenzenesulfonyl chloride (40 mg, 0.186 mmol, 2 eq). The mixture was stirred at rt under N2 for 3 h. The resulting mixture was purified by prep-HPLC (0.1% NH3·H2O) to afford 2,6-difluoro-N-(2-fluoro-4-methyl-3-(2-(methylamino)-8,9-dihydroimidazo[1′,2′:1,6]pyrido[2,3-d]pyrimidin-6-yl)phenyl)benzenesulfonamide (2.6 mg, 5.6% yield) as a yellow solid. ¹H NMR (400 MHz, CD₃OD): δ 8.37 (s, 1H), 7.52-7.48 (m, 1H), 7.36-7.26 (m, 4H), 7.00 (d, J=8.0 Hz, 1H), 4.41-4.36 (m, 2H), 4.01-3.96 (m, 2H), 3.00 (s, 3H), 2.16 (s, 3H). LCMS (M+H⁺) m/z: 501.0.

### Example 298: Preparation of N-(2-fluoro-4-methyl-3-(2-(methylamino)-8,9-dihydroimidazo[1′,2′:1,6]pyrido[2,3-d]pyrimidin-6-yl)phenyl)-2-(trifluoromethyl)benzenesulfonamide (Compound 298)

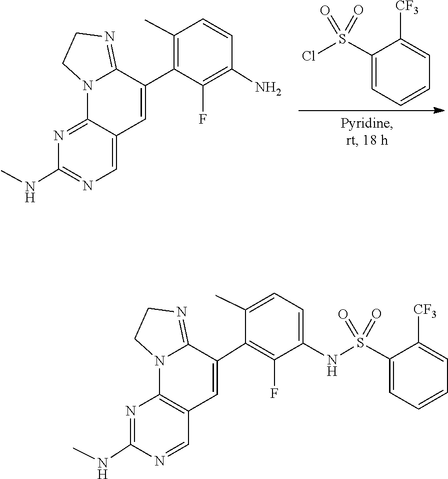 [SMILES: CNc1ncc2c(n1)N1CCN=C1C(c1c(C)ccc(N)c1F)=C2 ; O=S(=O)(Cl)c1ccccc1C(F)(F)F ; CNc1ncc2c(n1)N1CCN=C1C(c1c(C)ccc(NS(=O)(=O)c3ccccc3C(F)(F)F)c1F)=C2]

### Step 1: Synthesis of N-(2-fluoro-4-methyl-3-(2-(methylamino)-8,9-di hydroimidazo[1′,2′:1,6]pyrido[2,3-d]pyrimidin-6-yl)phenyl)-2-(trifluoromethyl)benzenesulfonamide

To a mixture of 6-(3-amino-2-fluoro-6-methylphenyl)-N-methyl-8,9-dihydroimidazo[1′,2′:1,6]pyrido[2,3-d]pyrimidin-2-amine (40 mg, 0.12 mmol) in Pyridine (2 mL), was added 2-(trifluoromethyl)benzenesulfonyl chloride (60 mg, 0.24 mmol). The mixture was stirred at 20° C. for 16 h. Concentration and purification by prep-HPLC (0.1% NH3·H2O) afforded N-(2-fluoro-4-methyl-3-(2-(methylamino)-8,9-di hydroimidazo[1′,2′:1,6]pyrido[2,3-d]pyrimidin-6-yl)phenyl)-2-(trifluoromethyl)benzenesulfonamide (1.0 mg, 2% yield) as a yellow solid. ¹H NMR (400 MHz, CD₃OD): δ 8.31 (s, 1H), 8.04 (d, J=7.2 Hz, 1H), 7.91 (d, J=7.2 Hz, 1H), 7.76-7.66 (m, 2H), 7.30-7.26 (m, 2H), 7.01 (d, J=8.0 Hz, 1H), 4.90-4.88 (m, 2H), 4.00-3.96 (m, 2H), 2.99 (s, 3H), 2.16 (s, 3H). LCMS (M+H⁺) m/z: 533.0.

### Example 299: Preparation of N-(2-fluoro-4-methyl-3-(2-(methylamino)-8,9-dihydroimidazo[1′,2′:1,6]pyrido[2,3-d]pyrimidin-6-yl)phenyl)-2-(trifluoromethoxy)benzenesulfonamide (Compound 299)

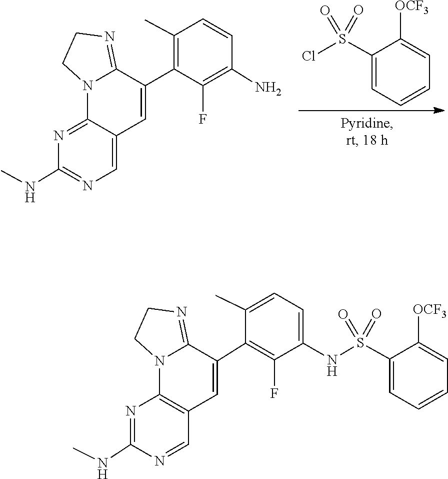 [SMILES: CNc1ncc2c(n1)N1CCN=C1C(c1c(C)ccc(N)c1F)=C2 ; O=S(=O)(Cl)c1ccccc1OC(F)(F)F ; CNc1ncc2c(n1)N1CCN=C1C(c1c(C)ccc(NS(=O)(=O)c3ccccc3OC(F)(F)F)c1F)=C2]

### Step 1: Synthesis of N-(2-fluoro-4-methyl-3-(2-(methylamino)-8,9-di hydroimidazo[1′,2′:1,6]pyrido[2,3-d]pyrimidin-6-yl)phenyl)-2-(trifluoromethoxy)benzenesulfonamide

To a mixture of 6-(3-amino-2-fluoro-6-methylphenyl)-N-methyl-8,9-dihydroimidazo[1′,2′:1,6]pyrido[2,3-d]pyrimidin-2-amine (40 mg, 0.12 mmol) in Pyridine (2 mL), was added 2-(trifluoromethoxy)benzenesulfonyl chloride (60 mg, 0.24 mmol). The mixture was stirred at 20° C. for 16 h. Concentration and purification by prep-HPLC (0.1% NH3·H2O) afforded N-(2-fluoro-4-methyl-3-(2-(methylamino)-8,9-dihydroimidazo[1′,2′:1,6]pyrido[2,3-d]pyrimidin-6-yl)phenyl)-2-(trifluoromethoxy)benzenesulfonamide (2.1 mg, 3% yield) as a yellow solid. ¹H NMR (400 MHz, CD₃OD): δ 8.27 (s, 1H), 7.90 (d, J=8.0 Hz, 1H), 7.70-7.66 (m, 1H), 7.47 (d, J=7.6 Hz, 1H), 7.40 (t, J=8.0 Hz, 1H), 7.28 (t, J=8.0 Hz, 1H), 7.18-7.16 (m, 1H), 6.99 (d, J=8.8 Hz, 1H), 4.30-4.25 (m, 2H), 3.96 (t, J=10.0 Hz, 2H), 2.99 (s, 3H), 2.15 (s, 3H). LCMS (M+H⁺) m/z: 549.0.

### Example 300: Preparation of 2-fluoro-N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)benzenesulfonamide (Compound 300)

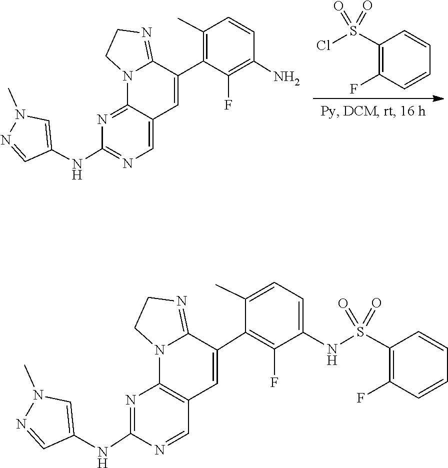 [SMILES: Cc1ccc(N)c(F)c1C1=Cc2cnc(Nc3cnn(C)c3)nc2N2CCN=C12 ; O=S(=O)(Cl)c1ccccc1F ; Cc1ccc(NS(=O)(=O)c2ccccc2F)c(F)c1C1=Cc2cnc(Nc3cnn(C)c3)nc2N2CCN=C12]

### Step 1: Synthesis of 2-fluoro-N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)benzenesulfonamide

To a solution of 6-(3-amino-2-fluoro-6-methylphenyl)-N-(1-methyl-1H-pyrazol-4-yl)-8,9-dihydroimidazo[1′,2′:1,6]pyrido[2,3-d]pyrimidin-2-amine (40 mg, 0.1 mmol, 1.0 eq) in DCM (5 mL) was added pyridine (0.1 mL) and 2-fluorobenzenesulfonyl chloride (30 mg, 0.15 mmol, 1.5 eq). The mixture was stirred at 20° C. under N₂ for 16 h. LCMS showed the reaction was OK. Concentration in vacuum and purification by Prep-HPLC (0.1% TFA) afforded 2-fluoro-N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)benzenesulfonamide (9.0 mg, 16% yield) as a yellow solid. ¹H NMR (400 MHz, CD₃OD): δ 8.87 (s, 1H), 8.04-8.03 (m, 2H), 7.85-7.63 (m, 3H), 7.46-7.42 (m, 1H), 7.32-7.27 (m, 2H), 7.16-7.14 (m, 1H), 4.91-4.82 (m, 2H), 4.18-4.11 (m, 2H), 3.90 (s, 3H), 2.20 (s, 3H). LCMS (M+H⁺) m/z: 549.5.

### Example 301: Preparation of 2,5-difluoro-N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)benzenesulfonamide (Compound 301)

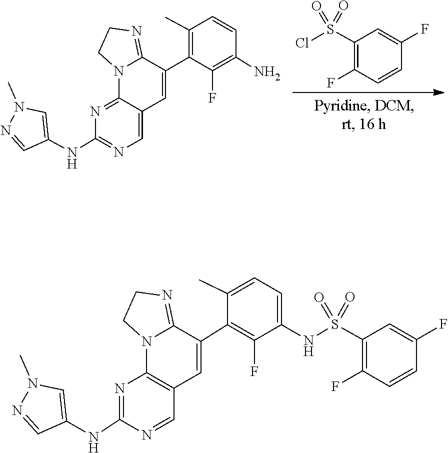 [SMILES: Cc1ccc(N)c(F)c1C1=Cc2cnc(Nc3cnn(C)c3)nc2N2CCN=C12 ; O=S(=O)(Cl)c1cc(F)ccc1F ; Cc1ccc(NS(=O)(=O)c2cc(F)ccc2F)c(F)c1C1=Cc2cnc(Nc3cnn(C)c3)nc2N2CCN=C12]

### Step 1: Synthesis of 2,5-difluoro-N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)benzenesulfonamide

To a solution of 6-(3-amino-2-fluoro-6-methylphenyl)-N-(1-methyl-1H-pyrazol-4-yl)-8,9-dihydroimidazo[1′,2′:1,6]pyrido[2,3-d]pyrimidin-2-amine (60 mg, 0.15 mmol, 1.0 eq) in DCM (5 mL) was added pyridine (0.1 mL) and 2,5-difluorobenzenesulfonyl chloride (65 mg, 0.3 mmol, 2 eq). The mixture was stirred at 20° C. under N2 for 16 h. LCMS showed the reaction was OK. The mixture was concentrated in vacuum and purified by Prep-HPLC (0.1% TFA) to afford 2,5-difluoro-N-(2-fluoro-4-methyl-3-(2-((1-methyl-1H-pyrazol-4-yl)amino)-8,9-dihydroimidazo[1′,2′:1,6]pyrido[2,3-d]pyrimidin-6-yl)phenyl)benzenesulfonamide (14.1 mg, 17% yield) as a yellow solid. ¹H NMR (400 MHz, CD₃OD): δ 8.88 (s, 1H), 8.06-8.04 (m, 2H), 7.77 (s, 1H), 7.57-7.53 (m, 1H), 7.47-7.39 (m, 3H), 7.20-7.18 (m, 1H), 4.91-4.88 (m, 2H), 4.18-4.11 (m, 2H), 3.89 (s, 3H), 2.20 (s, 3H). LCMS (M+H⁺) m/z: 567.4.

### Example 302: Preparation of 2,5-difluoro-N-(2-fluoro-4-methyl-3-(2-(oxetan-3-ylamino)-8,9-dihydroimidazo[1′,2′:1,6]pyrido[2,3-d]pyrimidin-6-yl)phenyl)benzenesulfonamide (Compound 302)

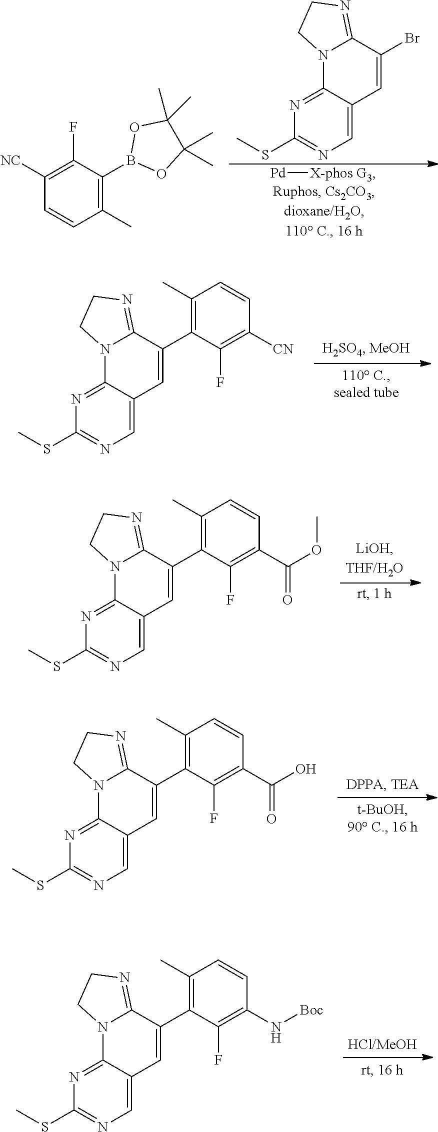

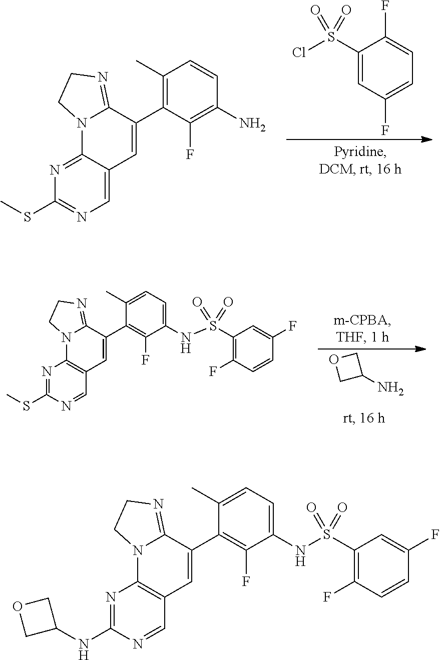 [SMILES: CSc1ncc2c(n1)N1CCN=C1C(c1c(C)ccc(N)c1F)=C2 ; O=S(=O)(Cl)c1cc(F)ccc1F ; CSc1ncc2c(n1)N1CCN=C1C(c1c(C)ccc(NS(=O)(=O)c3cc(F)ccc3F)c1F)=C2 ; NC1COC1 ; Cc1ccc(NS(=O)(=O)c2cc(F)ccc2F)c(F)c1C1=Cc2cnc(NC3COC3)nc2N2CCN=C12]

### Step 1: Synthesis of 2-fluoro-4-methyl-3-(2-(methylthio)-8, 9-dihydroimidazo[1′, 2′:1, 6]pyrido[2, 3-d]pyrimidin-6-yl)benzonitrile

To a solution of 6-bromo-2-(methylthio)-8,9-dihydroimidazo[1′,2′:1,6]pyrido[2,3-d]pyrimidine (0.66 g, 2.23 mmol) in dioxane/H₂O (20 mL/4 mL) was added 2-fluoro-4-methyl-3-(4,4,5,5-tetramethyl-1,3,2-dioxaborolan-2-yl)benzonitrile (760 mg, 2.90 mmol), Cs₂CO₃ (2.2 g, 6.69 mmol), Ruphos (210 mg, 0.446 mmol) and Pd-X-phos G3 (380 mg, 0.446 mmol). The reaction mixture was stirred at 110° C. under N₂ for 16 h. Concentration in vacuum and purification on silica gel column chromatography (DCM/MeOH=10:1) afforded 2-fluoro-4-methyl-3-(2-(methylthio)-8, 9-dihydroimidazo[1′, 2′:1, 6]pyrido[2, 3-d]pyrimidin-6-yl) benzonitrile (0.7 g, 89.4% yield) as a yellow solid.

### Step 2: Synthesis of methyl 2-fluoro-4-methyl-3-(2-(methylthio)-8, 9-dihydroimidazo [1′, 2′:1, 6]pyrido[2, 3-d]pyrimidin-6-yl)benzoate

To a solution of 2-fluoro-4-methyl-3-(2-(methylthio)-8, 9-dihydroimidazo[1′, 2′:1, 6]pyrido[2, 3-d]pyrimidin-6-yl)benzonitrile (0.7 g, 1.99 mmol) in MeOH (15 mL) was added H2SO4 (3 mL), the reaction mixture was stirred at 100° C. under sealed tube for 36 h. Concentration in vacuum and purification on column chromatography (0.1% NH3H2O) afforded methyl 2-fluoro-4-methyl-3-(2-(methylthio)-8, 9-dihydroimidazo[1′, 2′:1, 6]pyrido[2, 3-d]pyrimidin-6-yl)benzoate (300 mg, 39.2% yield) as a yellow solid.

### Step 3: Synthesis of 2-fluoro-4-methyl-3-(2-(methylthio)-8, 9-dihydroimidazo[1′, 2′:1, 6]pyrido[2, 3-d]pyrimidin-6-yl)benzoic acid

To a solution of methyl 2-fluoro-4-methyl-3-(2-(methylthio)-8, 9-dihydroimidazo[1′, 2′:1, 6]pyrido[2, 3-d]pyrimidin-6-yl)benzoate (300 mg, 0.78 mmol) in MeOH/H2O (5 mL/5 mL) was added LiOH (66 mg, 1.56 mmol). The reaction mixture was stirred at rt for 3 h, Concentration in vacuum and purification on column chromatography afforded 2-fluoro-4-methyl-3-(2-(methylthio)-8, 9-dihydroimidazo[1′, 2′:1, 6]pyrido[2, 3-d]pyrimidin-6-yl) benzoic acid (210 mg, 72.8% yield) as a yellow solid.

### Step 4: Synthesis of tert-butyl (2-fluoro-4-methyl-3-(2-(methylthio)-8, 9-dihydroimidazo[1′, 2′:1, 6]pyrido[2, 3-d]pyrimidin-6-yl)phenyl) carbamate

To a solution of 2-fluoro-4-methyl-3-(2-(methylthio)-8, 9-dihydroimidazo[1′, 2′:1, 6]pyrido[2, 3-d]pyrimidin-6-yl)benzoic acid (210 mg, 0.57 mmol) in t-BuOH (10 mL) was added DPPA (314 mg, 1.14 mmol) and TEA (172 mg, 1.71 mmol). The reaction mixture was stirred at 90° C. for 16 h. Concentration in vacuum afforded tert-butyl (2-fluoro-4-methyl-3-(2-(methylthio)-8, 9-dihydroimidazo[1′, 2′:1, 6]pyrido[2, 3-d]pyrimidin-6-yl)phenyl) carbamate (300 mg, crude) as a yellow solid, which was used for next step directly.

### Step 5: Synthesis of 2-fluoro-4-methyl-3-(2-(methylthio)-8, 9-dihydroimidazo[1′, 2′:1, 6]pyrido[2, 3-d]pyrimidin-6-yl) aniline

A solution of tert-butyl (2-fluoro-4-methyl-3-(2-(methylthio)-8, 9-dihydroimidazo[1′, 2′:1, 6]pyrido[2, 3-d]pyrimidin-6-yl)phenyl) carbamate (300 mg, crude) in MeOH/HCl (5 mL) was stirred at rt for 16 h. Concentration in vacuum and purification on column chromatography afforded 2-fluoro-4-methyl-3-(2-(methylthio)-8, 9-dihydroimidazo[1′, 2′:1, 6]pyrido[2, 3-d]pyrimidin-6-yl) aniline (120 mg, 45.1% yield) as a yellow solid.

### Step 6: Synthesis of 2,5-difluoro-N-(2-fluoro-4-methyl-3-(2-(methylthio)-8, 9-dihydroimidazo[1′,2′:1,6]pyrido[2,3-d]pyrimidin-6-yl)phenyl)benzenesulfonamide

To a solution of 2-fluoro-4-methyl-3-(2-(methylthio)-8, 9-dihydroimidazo[1′, 2′:1, 6]pyrido[2, 3-d]pyrimidin-6-yl) aniline (60 mg, 0.176 mmol) in DCM (5 mL) was added 2,5-difluorobenzenesulfonyl chloride (75 mg, 0.352 mmol) and pyridine (0.5 mL). The reaction was stirred at rt for 16 h. The resulting mixture was purified on column chromatography afforded 2,5-difluoro-N-(2-fluoro-4-methyl-3-(2-(methylthio)-8, 9-dihydroimidazo[1′,2′:1,6]pyrido[2,3-d]pyrimidin-6-yl)phenyl)benzenesulfonamide (45 mg, 22.3% yield) as a yellow solid.

### Step 7: Synthesis of 6-bromo-2-(methylsulfinyl)-8, 9-dihydroimidazo[1′, 2′:1, 6]pyrido[2, 3-d]pyrimidine

To a solution of 2,5-difluoro-N-(2-fluoro-4-methyl-3-(2-(methylthio)-8, 9-dihydroimidazo[1′,2′:1,6]pyrido[2,3-d]pyrimidin-6-yl)phenyl)benzenesulfonamide (45 mg, 0.087 mmol, 1.0 eq) in DCM (10 mL) was added m-CPBA (38 mg, 0.218 mmol, 2.5 eq) at 0° C. The reaction mixture was stirred at 0° C. for 1 h. Then oxetan-3-amine (32 mg, 0.437 mmol, 5.0 eq) was added, the reaction mixture was stirred at 20° C. for 16 h. LCMS showed the reaction was completed. The reaction mixture was diluted with DCM (50 mL) and washed with H₂O (50 mL), brine (20 mL). Concentration in vacuum and purification by prep-HPLC (0.1% NH₃·H₂O) afforded 6-bromo-2-(methylsulfinyl)-8, 9-dihydroimidazo[1′, 2′:1, 6]pyrido[2, 3-d]pyrimidine (4.1 mg, 10.8% yield) as a yellow solid. ¹H NMR (400 MHz, CD₃OD): δ 8.32 (s, 1H), 7.51-7.47 (m, 1H), 7.36-7.24 (m, 4H), 7.00 (d, J=8.4 Hz, 1H), 5.12 (t, J=7.2 Hz, 1H), 4.95 (t, J=6.8 Hz, 2H), 4.68 (t, J=6.8 Hz, 2H), 4.26 (t, J=9.6 Hz, 2H), 3.97 (t, J=10.0 Hz, 2H), 2.16 (s, 3H). LCMS (M+H⁺) m/z: 543.1.

### Example 303: Preparation of 2-chloro-N-(2-fluoro-4-methyl-3-(2-(oxetan-3-ylamino)-8,9-dihydroimidazo[1′,2′:1,6]pyrido[2,3-d]pyrimidin-6-yl)phenyl)benzenesulfonamide (Compound 303)

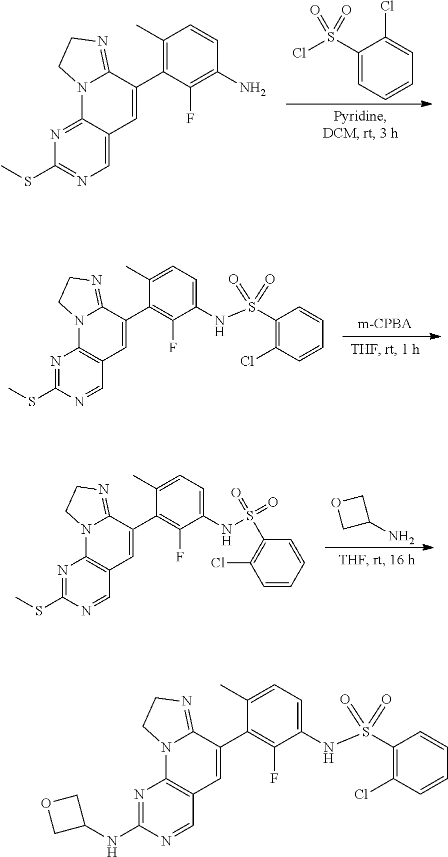 [SMILES: CSc1ncc2c(n1)N1CCN=C1C(c1c(C)ccc(N)c1F)=C2 ; O=S(=O)(Cl)c1ccccc1Cl ; CSc1ncc2c(n1)N1CCN=C1C(c1c(C)ccc(NS(=O)(=O)c3ccccc3Cl)c1F)=C2 ; CSc1ncc2c(n1)N1CCN=C1C(c1c(C)ccc(NS(=O)(=O)c3ccccc3Cl)c1F)=C2 ; NC1COC1 ; Cc1ccc(NS(=O)(=O)c2ccccc2Cl)c(F)c1C1=Cc2cnc(NC3COC3)nc2N2CCN=C12]

### Step 1: Synthesis of 2-fluoro-4-methyl-3-(2-(methylthio)-8, 9-dihydroimidazo[1′, 2′:1, 6]pyrido[2, 3-d]pyrimidin-6-yl) aniline

To a mixture of 2-fluoro-4-methyl-3-(2-(methylthio)-8,9-dihydroimidazo[1′,2′:1,6]pyrido[2,3-d]pyrimidin-6-yl)aniline (60 mg, 0.176 mmol, 1.0 eq) in DCM (5 mL) was added 2-chlorobenzenesulfonyl chloride (74 mg, 0.352 mmol, 2.0 eq) and pyridine (0.2 mL). The mixture was stirred at 20° C. for 3 h. LCMS show the reaction was OK. The reaction mixture was diluted with DCM (50 mL) and washed with H₂O (50 mL), brine (20 mL). Concentration in vacuum and purification by prep-HPLC (0.1% NH₃·H₂O) afforded 2-fluoro-4-methyl-3-(2-(methylthio)-8, 9-dihydroimidazo[1′, 2′:1, 6]pyrido[2, 3-d]pyrimidin-6-yl) aniline (45 mg, 49.6% yield) as a yellow solid.

### Step 2: Synthesis of 2-chloro-N-(2-fluoro-4-methyl-3-(2-(oxetan-3-ylamino)-8,9-dihydroimidazo[1′,2′:1,6]pyrido[2,3-d]pyrimidin-6-yl)phenyl)benzenesulfonamide

To a solution of 2-fluoro-4-methyl-3-(2-(methylthio)-8, 9-dihydroimidazo[1′, 2′:1, 6]pyrido[2, 3-d]pyrimidin-6-yl) aniline (45 mg, 0.087 mmol, 1.0 eq) in DCM (10 mL) was added m-CPBA (38 mg, 0.218 mmol, 2.5 eq) at 0° C. The reaction mixture was stirred at 0° C. for 1 h. Then oxetan-3-amine (32 mg, 0.437 mmol, 5.0 eq) was added, the reaction mixture was stirred at 20° C. for 16 h. LCMS showed the reaction was completed. The reaction mixture was diluted with DCM (50 mL) and washed with H₂O (50 mL), brine (20 mL). Concentration in vacuum and purification by prep-HPLC (0.1% NH3·H2O) afforded 2-chloro-N-(2-fluoro-4-methyl-3-(2-(oxetan-3-ylamino)-8,9-dihydroimidazo[1′,2′:1,6]pyrido[2,3-d]pyrimidin-6-yl)phenyl) benzenesulfonamide (5.1 mg, 10.8% yield) as a yellow solid. ¹H NMR (400 MHz, CD₃OD): δ 8.23 (s, 1H), 7.97 (d, J=7.6 Hz, 1H), 7.56-7.52 (m, 2H), 7.42-7.38 (m, 1H), 7.25 (t, J=8.0 Hz, 1H), 7.12 (s, 1H), 6.95 (d, J=8.8 Hz, 1H), 5.13-5.09 (m, 1H), 4.94 (t, J=6.8 Hz, 2H), 4.67 (t, J=6.4 Hz, 2H), 4.19 (t, J=9.6 Hz, 2H), 3.94 (t, J=10.0 Hz, 2H), 2.13 (s, 3H). LCMS (M+H⁺) m/z: 541.1.

### Example 304: Preparation of N-(4-((2-(methylamino)-8,9-dihydroimidazo[1′,2′:1,6]pyrido[2,3-d]pyrimidin-6-yl)oxy)phenyl)-4-(trifluoromethyl)picolinamide (Compound 304)

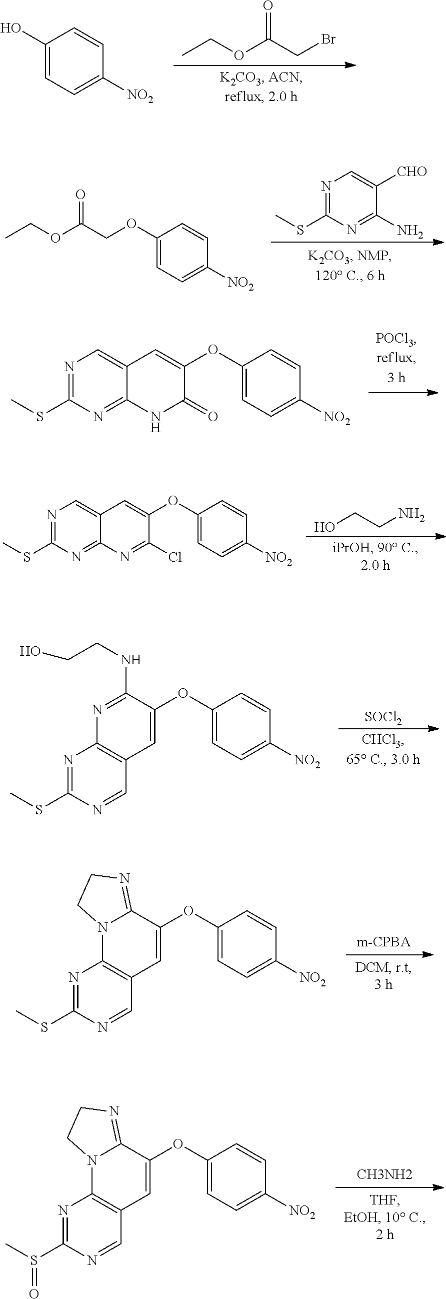

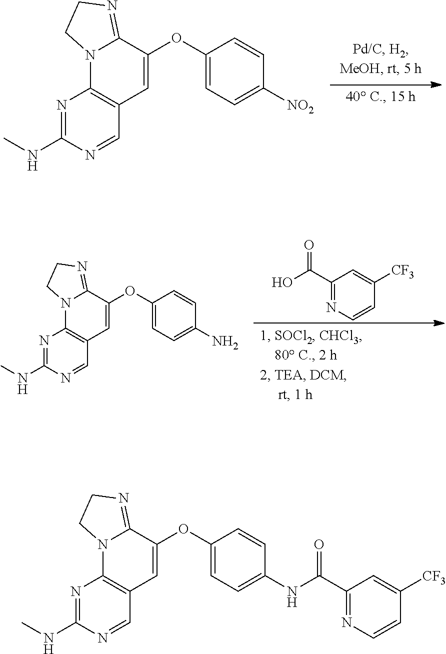 [SMILES: CNc1ncc2c(n1)N1CCN=C1C(Oc1ccc([N+](=O)[O-])cc1)=C2 ; CNc1ncc2c(n1)N1CCN=C1C(Oc1ccc(N)cc1)=C2 ; O=C(O)c1cc(C(F)(F)F)ccn1 ; CNc1ncc2c(n1)N1CCN=C1C(Oc1ccc(NC(=O)c3cc(C(F)(F)F)ccn3)cc1)=C2]

### Step 1: Synthesis of ethyl 2-(4-nitrophenoxy)acetate

A mixture of 4-nitrophenol (14 g, 100 mmol), ethyl 2-bromoacetate (16.7 g, 100 mmol), K₂CO₃ (34.5 g, 250 mmol) in MeCN (180 mL) was stirred for 3 h at 85° C. The reaction was filtered and concentrated to give ethyl 2-(4-nitrophenoxy)acetate (18.6 g) as solid. LCMS (M+H⁺) m/z: 225.9

### Step 2: Synthesis of 2-(methylthio)-6-(4-nitrophenoxy)pyrido[2,3-d]pyrimidin-7(8H)-one

A mixture of ethyl 2-(4-nitrophenoxy)acetate (4.22 g, 20 mmol), 4-amino-2-(methylthio)pyrimidine-5-carbaldehyde (3.40 g, 20 mmol), K2CO3 (8.28 g, 60 mmol) in NMP (60 mL) was stirred at 120° C. for 16 h. The reaction was filtered and poured into water, the pH was adjusted to 7 with 2N HCl. The resulting solid was filtered and dried to give 2-(methylthio)-6-(4-nitrophenoxy)pyrido[2,3-d]pyrimidin-7(8H)-one (3.8 g) as orange solid. LCMS (M+H⁺) m/z: 330.9

### Step 3: Synthesis of 7-chloro-2-(methylthio)-6-(4-nitrophenoxy)pyrido[2,3-d]pyrimidine

To a mixture of 2-(methylthio)-6-(4-nitrophenoxy)pyrido[2,3-d]pyrimidin-7(8H)-one (2.0 g, 6.0 mmol) was added POCl₃ (12.0 mL) at rt. The reaction mixture was stirred for 5 h at 110° C. The reaction mixture was concentrated and poured into NaHCO₃ aq, extracted with EA (100 mL×2). The combined organic phase was washed with water, dried over with Na₂SO₄, concentrated to give 7-chloro-2-(methylthio)-6-(4-nitrophenoxy)pyrido[2,3-d]pyrimidine (1.2 g, 60% yield) as white solid. LCMS (M+H⁺) m/z 348.9.

### Step 4: Synthesis of 2-((2-(methylthio)-6-(4-nitrophenoxy)pyrido[2,3-d]pyrimidin-7-yl)amino)ethan-1-ol

A mixture of 7-chloro-2-(methylthio)-6-(4-nitrophenoxy)pyrido[2,3-d]pyrimidine (1.0 g, 3.0 mmol), ethanolamine (720 mg, 12.0 mmol) in i-PrOH (20 mL) was stirred for 3 h at 80° C. The reaction was concentrated and purified by flash (PE:EA=3:1) to give 2-((2-(methylthio)-6-(4-nitrophenoxy)pyrido[2,3-d]pyrimidin-7-yl)amino)ethan-1-ol (860 mg, 77% yield) as solid. LCMS (M+H⁺) m/z: 373.9

### Step 5: Synthesis of 2-(methylthio)-6-(4-nitrophenoxy)-8,9-dihydroimidazo[1′,2′:1,6]pyrido[2,3-d]pyrimidine

To a mixture of 2-((2-(methylthio)-6-(4-nitrophenoxy)pyrido[2,3-d]pyrimidin-7-yl)amino)ethan-1-ol (370 mg, 1.0 mmol) in CHCl₃ (10 mL) was added SOCl₂ (360 mg, 3.0 mmol) at rt. The reaction mixture was stirred for 2 h at 60° C. The reaction was concentrated and purified by flash (PE:EA=3:1) to give 2-(methylthio)-6-(4-nitrophenoxy)-8,9-dihydroimidazo[1′,2′:1,6]pyrido[2,3-d]pyrimidine (260 mg, 73% yield) as solid. LCMS (M+H⁺) m/z: 355.9.

### Step 6: Synthesis of 2-(methylsulfinyl)-6-(4-nitrophenoxy)-8,9-dihydroimidazo[1′,2′:1,6]pyrido[2,3-d]pyrimidine

To a mixture of 2-(methylthio)-6-(4-nitrophenoxy)-8,9-dihydroimidazo[1′,2′:1,6]pyrido[2,3-d]pyrimidine (520 mg, 1.5 mmol) in DCM (20 mL) was added m-CPBA (645 mg, 3.75 mmol) at rt. The reaction mixture was stirred for 3 h at 25° C. The reaction was concentrated and purified by flash (DCM:Methanol=40:1) to give 2-(methylsulfinyl)-6-(4-nitrophenoxy)-8,9-dihydroimidazo[1′,2′:1,6]pyrido[2,3-d]pyrimidine (430 mg, 77% yield) as solid. LCMS (M+H⁺) m/z: 371.9.

### Step 7: Synthesis of N-methyl-6-(4-nitrophenoxy)-8,9-dihydroimidazo[1′,2′:1,6]pyrido[2,3-d]pyrimidin-2-amine

To a mixture of 2-(methylsulfinyl)-6-(4-nitrophenoxy)-8,9-dihydroimidazo[1′,2′:1,6]pyrido[2,3-d]pyrimidine (430 mg, 1.15 mmol) in THF (2.0 mL) was added 30% MeNH₂ in ethanol (2.0 mL) at rt. The reaction mixture was stirred for 2 h at 30° C. The reaction was cooled to room temperature and filtered to give N-methyl-6-(4-nitrophenoxy)-8,9-dihydroimidazo[1′,2′:1,6]pyrido[2,3-d]pyrimidin-2-amine (310 mg, 80% yield) as solid. LCMS (M+H⁺) m/z: 339.0

### Step 8: Synthesis of 6-(4-aminophenoxy)-N-methyl-8,9-dihydroimidazo[1′,2′:1,6]pyrido[2,3-d]pyrimidin-2-amine

A mixture of N-methyl-6-(4-nitrophenoxy)-8,9-dihydroimidazo[1′,2′:1,6]pyrido[2,3-d]pyrimidin-2-amine (100 mg, 0.3 mmol) and Pd/C (20 mg) in MeOH (10 mL) was stirred at 30° C. under H₂ for 5 h. The reaction was stirred at 50° C. for 16 h. The reaction was filtered and concentrated to give 6-(4-aminophenoxy)-N-methyl-8,9-dihydroimidazo[1′,2′:1,6]pyrido[2,3-d]pyrimidin-2-amine (74 mg, 80% yield) as solid. LCMS (M+H⁺) m/z: 309.0

### Step 9: Synthesis of N-(4-((2-(methylamino)-8,9-dihydroimidazo[1′,2′:1,6]pyrido[2,3-d]pyrimidin-6-yl)oxy)phenyl)-4-(trifluoromethyl)picolinamide

To a mixture of 4-(trifluoromethyl)picolinic acid (223 mg, 1.0 mmol) in CHCl₃ (1.0 mL), was added SOCl₂ (1.0 mL) at rt. The reaction mixture was stirred for 2 h at 80° C. The reaction was concentrated to give crude 4-(trifluoromethyl)picolinoyl chloride. To a mixture of 6-(4-aminophenoxy)-N-methyl-8,9-dihydroimidazo[1′,2′:1,6]pyrido[2,3-d]pyrimidin-2-amine (60 mg, 0.2 mmol), DIPEA (78 mg, 0.6 mmol) in DCM (5.0 mL) was added a solution of crude 4-(trifluoromethyl)picolinoyl chloride (50 mg, 0.24 mmol) in DCM (1.0 mL). The reaction was stirred for 2 hour at 25° C. The reaction was concentrated and purified by Prep-HPLC (NH₄HCO₃) to give N-(4-((2-(methylamino)-8,9-dihydroimidazo[1′,2′:1,6]pyrido[2,3-d]pyrimidin-6-yl)oxy)phenyl)-4-(trifluoromethyl)picolinamide (23.6 mg, 24% yield) as white solid. ¹H NMR (400 MHz, DMSO-d₆): δ 10.86 (s, 1H), 9.04 (d, J=4.4 Hz, 1H), 8.36 (s, 1H), 8.17 (s, 1H), 8.10-8.09 (m, 1H), 7.93-7.90 (m, 2H), 7.34 (br, 1H), 7.11-7.08 (m, 2H), 6.88 (br, 1H), 4.09-3.93 (m, 4H), 2.82 (d, J=4.0 Hz, 3H). LCMS (M+H⁺) m/z: 482.0.

### Example 305: Preparation of N-(3-((6-(2,4-difluorophenoxy)-8,9-dihydroimidazo[1′,2′:1,6]pyrido[2,3-d]pyrimidin-2-yl)amino)phenyl)acrylamide (Compound 305)

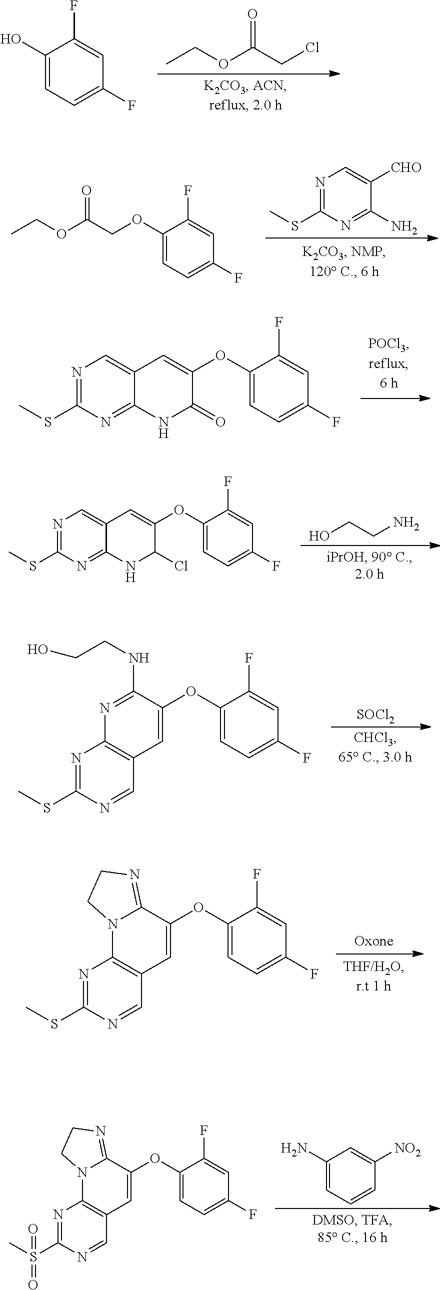

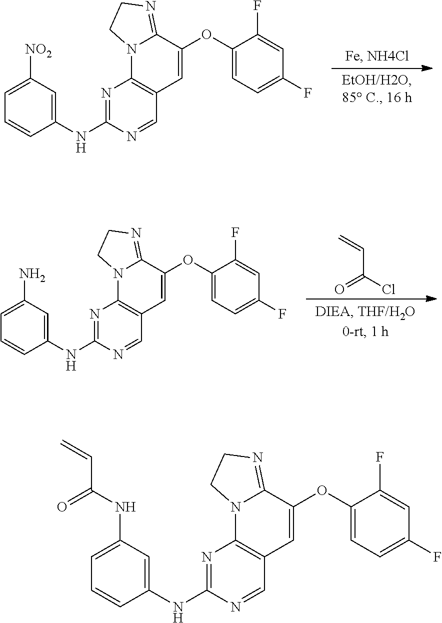

### Step 1: Synthesis of ethyl 2-(2,4-difluorophenoxy)acetate

A solution of 2,4-difluorophenol (10 g, 76.9 mmol), ethyl 2-chloroacetate (9.4 g, 76.9 mmol) in CH₃CN (300 mL) was added K₂CO₃ (12.7 g, 92.3 mmol). The reaction mixture was stirred at 80° C. for 16 hours. The reaction mixture was concentrated in vacuum. The residue was diluted in water (200 mL) and extracted with DCM (200 ML×2). The combined organic layer was washed with brine (200 mL) and dried over Na₂SO₄, filtered, concentrated to give ethyl 2-(2,4-difluorophenoxy)acetate (17 g, crude) as light yellow oil. ¹H NMR (400 MHz, DMSO-d₆): δ 7.33-7.27 (m, 1H), 7.19-7.15 (m, 1H), 7.02-6.97 (m, 1H), 4.86 (s, 2H), 4.20-4.14 (m, 2H), 1.24-1.19 (m, 3H),

### Step 2: Synthesis of 6-(2,4-Difluorophenoxy)-2-(methylthio)pyrido[2,3-d]pyrimidin-7(8H)-one

K₂CO₃ (25.3 g, 183 mmol) was added to a solution of ethyl 2-(2,4-difluorophenoxy)acetate (18 g, 83.3 mmol), 4-amino-2-(methylthio)pyrimidine-5-carbaldehyde (12.7 g, 75 mmol) in NMP (150 mL). The reaction mixture was stirred at 100° C. for 16 hours. The reaction mixture was added dropwise to the ice water (1 L). The resulting precipitate was filtered and washed with water (100 mL). The solid was collected and dried to give 6-(2,4-Difluorophenoxy)-2-(methylthio)pyrido[2,3-d]pyrimidin-7(8H)-one. (11 g, 41% yield) as a yellow solid. LCMS (M+H⁺) m/z: 322.1

### Step 3: Synthesis of 7-Chloro-6-(2,4-difluorophenoxy)-2-(methylthio)pyrido[2,3-d]pyrimidine

A solution of 6-(2,4-difluorophenoxy)-2-(methylthio)pyrido[2,3-d]pyrimidin-7(8H)-one (11 g, 34.2 mmol) in POCl₃ (100 mL) was stirred at 95° C. for 16 hours. The reaction mixture was concentrated in vacuum. The residue was diluted in water (500 mL) and extracted with DCM (300 mL×2). The combined organic layer was washed with sat NaHCO₃ (500 mL) and concentrated to give the crude, which was purified on silica column (EA/PE=0% to 30%) to afford 7-chloro-6-(2,4-difluorophenoxy)-2-(methylthio)pyrido[2,3-d]pyrimidine (6.5 g, 56% yield) as a yellow solid. LCMS (M+H⁺) m/z: 340.1

### Step 4: Synthesis of 2-((6-(2,4-Difluorophenoxy)-2-(methylthio)pyrido[2,3-d]pyrimidin-7-yl)amino)ethan-1-ol

2-Aminoethan-1-ol (1.75 g, 28.7 mmol) was added to a solution of 7-chloro-6-(2,4-difluorophenoxy)-2-(methylthio)pyrido[2,3-d]pyrimidine (6.5 g, 19 mmol) in i-PrOH (100 mL). The reaction mixture was stirred at 90° C. for 2 hours. The reaction mixture was concentrated to give the crude, which was purified on silica column (EA/PE=0% to 90%) to afford 2-((6-(2,4-difluorophenoxy)-2-(methylthio)pyrido[2,3-d]pyrimidin-7-yl)amino)ethan-1-ol (6.0 g, 86% yield) as a yellow solid. LCMS (M+H⁺) m/z: 364.9

### Step 5: Synthesis of 6-(2,4-Difluorophenoxy)-2-(methylthio)-8,9-dihydroimidazo[1′,2′:1,6]pyrido[2,3-d]pyrimidine

SOCl₂ (9.8 g, 82.3 mmol) was added to a solution of 2-((6-(2,4-difluorophenoxy)-2-(methylthio)pyrido[2,3-d]pyrimidin-7-yl)amino)ethan-1-ol (6.0 g, 16.5 mmol) in CHCl₃ (100 mL). The reaction mixture was stirred at 65° C. for 3 hours. The mixture was quenched with sat NaHCO₃ (200 mL) and extracted with DCM (150 mL×2). The combined organic layers was washed with brine (200 mL) and concentrated to give the crude product, which was purified on silica column (EA/PE=0% to 90%) to afford 6-(2,4-difluorophenoxy)-2-(methylthio)-8,9-dihydroimidazo[1′,2′:1,6]pyrido[2,3-d]pyrimidine (4.3 g, 75% yield) as a yellow solid. LCMS (M+H⁺) m/z: 347.1

### Step 6: Synthesis of 6-(2,4-Difluorophenoxy)-2-(methylsulfonyl)-8,9-dihydroimidazo [1′,2′:1,6]pyrido[2,3-d]pyrimidine

The solution of oxone (1.2 g, 1.95 mmol) in water (10 mL) was added dropwise to a mixture of 6-(2,4-difluorophenoxy)-2-(methylthio)-8,9-dihydroimidazo[1′,2′:1,6]pyrido[2,3-d]pyrimidine (270 mg, 0.78 mmol) in THF (10 mL) at 0° C. The reaction mixture was stirred at room temperature for 2 hours. The mixture was quenched with sat NaHCO₃ (20 mL) and extracted with DCM (20 mL×2). The combined organic layers were washed with brine (30 mL) and concentrated to give 6-(2,4-difluorophenoxy)-2-(methylsulfonyl)-8,9-dihydroimidazo [1′,2′:1,6]pyrido[2,3-d]pyrimidine (270 mg, 92% yield) as a brown solid. LCMS (M+H⁺) m/z: 378.9

### Step 7: Synthesis of 6-(2,4-Difluorophenoxy)-N-(3-nitrophenyl)-8,9-dihydroimidazo [1′,2′:1,6]pyrido[2,3-d]pyrimidin-2-amine

TFA (1.05 g, 9.1 mmol) was added to a solution of 3-nitroaniline (438 mg, 3.2 mol), 6-(2,4-difluorophenoxy)-2-(methylsulfonyl)-8,9-dihydroimidazo[1′,2′:1,6]pyrido[2,3-d]pyrimidine (1 g, 2.6 mmol) in DMSO (30 mL). The reaction mixture was stirred at 120° C. for 3 hours. The mixture was quenched with sat. NaHCO₃ (100 mL) and extracted with DCM (60 mL×3). The combined organic layers were washed with brine (80 mL) and concentrated to give the crude product, which was purified by Prep-HPLC (0.1% FA) to give 6-(2,4-Difluorophenoxy)-N-(3-nitrophenyl)-8,9-dihydroimidazo[1′,2′:1,6]pyrido[2,3-d]pyrimidin-2-amine (650 mg, 56% yield) as a brown solid. LCMS (M+H⁺) m/z: 437.1

### Step 8: Synthesis of N1-(6-(2,4-difluorophenoxy)-8,9-dihydroimidazo[1′,2′:1,6]pyrido[2,3-d]pyrimidin-2-yl)benzene-1,3-diamine

Fe powder (767 mg, 13.7 mmol) was added to a mixture of NH₄Cl (441 mg, 8.24 mol), 6-(2,4-difluorophenoxy)-N-(3-nitrophenyl)-8,9-dihydroimidazo[1′,2′:1,6]pyrido[2,3-d]pyrimidin-2-amine (600 mg, 1.37 mmol) in EtOH/H₂O (2:1) (30 mL). The reaction mixture was stirred at 85° C. for 16 hours. The mixture was filtered and the filtrate was quenched with water (50 mL) and extracted with DCM (30 mL×3). The combined organic layers was washed with brine (50 mL) and concentrated to give the crude product, which was purified by silica column (EA/PE=5% to 90%, DCM/MeOH=9:1) to afford N1-(6-(2,4-difluorophenoxy)-8,9-dihydroimidazo[1′,2′:1,6]pyrido[2,3-d]pyrimidin-2-yl)benzene-1,3-diamine (210 mg, 38% yield) as a yellow solid. LCMS (M+H⁺) m/z: 407.0

### Step 9: Synthesis of N-(3-((6-(2,4-Difluorophenoxy)-8,9-dihydroimidazo[1′,2′:1,6]pyrido[2,3-d]pyrimidin-2-yl)amino)phenyl)acrylamide formate

Acryloyl chloride (46.8 mg, 0.52 mmol) was added to the mixture of N1-(6-(2,4-difluorophenoxy)-8,9-dihydroimidazo[1′,2′:1,6]pyrido[2,3-d]pyrimidin-2-yl)benzene-1,3-diamine (210 mg, 0.52 mmol), DIEA (200 mg, 1.55 mmol) in THF/H₂O (1:1) (6 mL). The solution was stirred at room temperature for 1 hour. The mixture was quenched with sat. NaHCO₃ (20 mL) and extracted with DCM (20 mL×3). The combined organic layers was washed with brine (40 mL), dried over Na₂SO₄, filtered, concentrated to give the crude product, which was purified by Prep-HPLC (0.1% HCOOH) to give N-(3-((6-(2,4-Difluorophenoxy)-8,9-dihydroimidazo[1′,2′:1,6]pyrido[2,3-d]pyrimidin-2-yl)amino)phenyl)acrylamide formate (43.8 mg, 18.4% yield) as a white solid. ¹H NMR (400 MHz, DMSO-d₆): δ 10.08 (s, 1H), 9.73 (s, 1H), 8.28 (s, 1H), 8.23 (s, 1H), 8.15 (s, 1H), 7.51-7.42 (m, 2H), 7.36-7.34 (m, 1H), 7.28-7.21 (m, 2H), 7.15-7.09 (m, 1H), 6.81 (s, 1H), 6.51-6.44 (m, 1H), 6.27-6.23 (m, 1H), 5.74 (dd, J=10.0, 2.0 Hz, 1H), 4.20 (t, J=9.6 Hz, 2H), 4.03 (t, J=9.6 Hz, 2H). LCMS (M+H⁺) m/z: 461.4.

### Example 306: Preparation of N-(4-((6-(2,4-difluorophenoxy)-8,9-dihydroimidazo[1′,2′:1,6]pyrido[2,3-d]pyrimidin-2-yl)amino)phenyl)acrylamide (Compound 306)

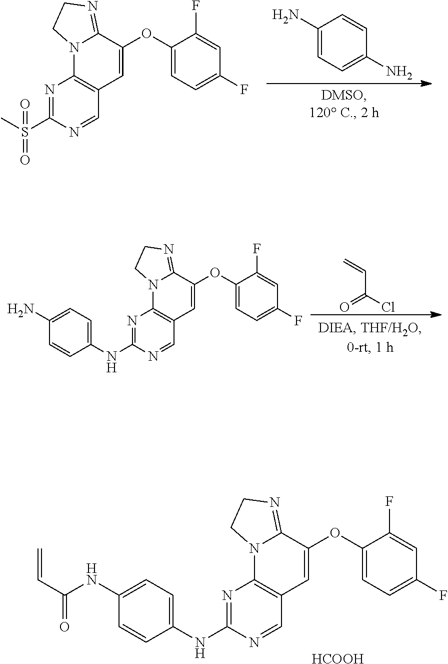

### Step 1: Synthesis of N1-(6-(2,4-Difluorophenoxy)-8,9-dihydroimidazo[1′,2′:1,6]pyrido[2,3-d]pyrimidin-2-yl)benzene-1,4-diamine

Benzene-1,4-diamine (106 mg, 0.98 mmol) was added to a solution of 6-(2,4-difluorophenoxy)-2-(methylsulfonyl)-8,9-dihydroimidazo[1′,2′:1,6]pyrido[2,3-d]pyrimidine 310 mg, 0.82 mmol) in DMSO (3 mL). The reaction mixture was stirred at 120° C. for 2 hours. The mixture was quenched with sat NaHCO₃ (50 mL) and extracted with DCM (30 mL×3). The combined organic layers were washed with brine (50 mL) and concentrated to give the crude product, which was purified by silica column (DCM:MeOH=9:1) to afford N1-(6-(2,4-Difluorophenoxy)-8,9-dihydroimidazo[1′,2′:1,6]pyrido[2,3-d]pyrimidin-2-yl)benzene-1,4-diamine (300 mg, 90% yield) as a dark brown solid. LCMS (M+H⁺) m/z: 407.1

### Step 2: Synthesis of N-(4-((6-(2,4-Difluorophenoxy)-8,9-dihydroimidazo[1′,2′:1,6]pyrido[2,3-d]pyrimidin-2-yl)amino)phenyl)acrylamide formate

Acryloyl chloride (60 mg, 0.66 mmol) was added to the solution of N1-(6-(2,4-difluorophenoxy)-8,9-dihydroimidazo[1′,2′:1,6]pyrido[2,3-d]pyrimidin-2-yl)benzene-1,4-diamine (270 mg, 0.66 mmol), DIEA (257 mg, 1.99 mmol) in THF/H₂O (1:1) (20 mL) at 0° C. The solution was stirred at room temperature for 1 hour. The mixture was quenched with sat NaHCO₃ (30 mL) and extracted with DCM (30 mL×3). The combined organic layers were washed with brine (50 mL), dried over Na₂SO₄, filtered, concentrated to give the crude product, which was purified by Prep-HPLC (0.1% HCOOH) to give N-(4-((6-(2,4-Difluorophenoxy)-8,9-dihydroimidazo[1′,2′:1,6]pyrido[2,3-d]pyrimidin-2-yl)amino)phenyl)acrylamide formate (5 mg, 1.5% yield) as a yellow solid. ¹H NMR (400 MHz, DMSO-d₆): δ 10.05 (s, 1H), 9.67 (s, 1H), 8.32 (s, 1H), 8.26 (s, 1H), 7.73 (d, J=8.8 Hz, 2H), 7.58 (d, J=9.2 Hz, 2H), 7.50-7.45 (m, 1H), 7.35-7.32 (m, 1H), 7.14-7.09 (m, 1H), 6.78 (s, 1H), 6.46-6.39 (m, 1H), 6.23 (dd, J=16.8, 2.0 Hz, 1H), 5.72 (dd, J=10.0, 2.0 Hz, 1H), 4.12 (t, J=9.2 Hz, 2H), 4.02 (t, J=9.2 Hz, 2H). LCMS (M+H⁺) m/z: 461.3.

### Example 307: Preparation of 1-(3-((6-(2,4-difluorophenoxy)-8,9-dihydroimidazo[1′,2′:1,6]pyrido[2,3-d]pyrimidin-2-yl)amino)pyrrolidin-1-yl)prop-2-en-1-one (Compound 307)

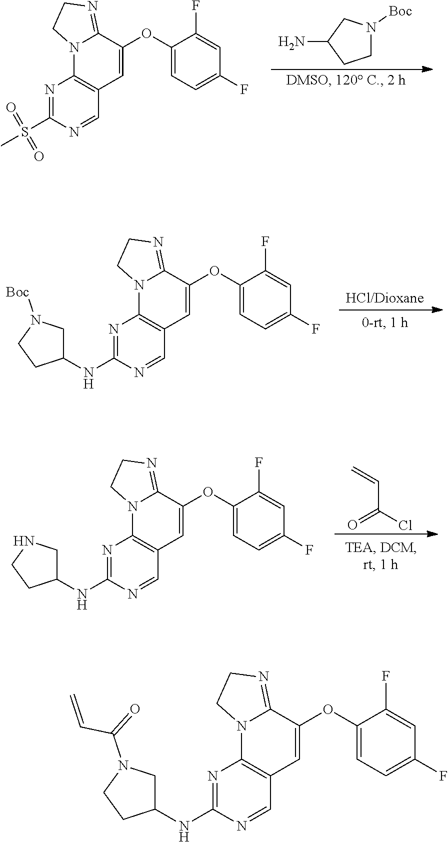

### Step 1: Synthesis of tert-butyl 3-((6-(2,4-difluorophenoxy)-8,9-dihydroimidazo[1′,2′:1,6]pyrido[2,3-d]pyrimidin-2-yl)amino)pyrrolidine-1-carboxylate

Tert-butyl 3-aminopyrrolidine-1-carboxylate (290 mg, 1.6 mmol) was added to a solution of 6-(2,4-difluorophenoxy)-2-(methylsulfonyl)-8,9-dihydroimidazo[1′,2′:1,6]pyrido[2,3-d]pyrimidine 500 mg, 1.3 mmol) in DMSO (5 mL). The reaction mixture was stirred at 120° C. for 2 hours. The mixture was quenched with water (50 mL) and extracted with DCM (50 mL×2). The combined organic layers were washed with brine (60 mL) and dried over Na₂SO₄, filtered, concentrated to give tert-butyl 3-((6-(2,4-difluorophenoxy)-8,9-dihydroimidazo[1′,2′:1,6]pyrido[2,3-d]pyrimidin-2-yl)amino)pyrrolidine-1-carboxylate (550 mg, crude). LCMS (M+H⁺) m/z: 485.1

### Step 2: Synthesis of 6-(2,4-Difluorophenoxy)-N-(pyrrolidin-3-yl)-8,9-dihydroimidazo [1′,2′:1,6]pyrido[2,3-d]pyrimidin-2-amine

The solution of tert-butyl 3-((6-(2,4-difluorophenoxy)-8,9-dihydroimidazo[1′,2′:1,6]pyrido[2,3-d]pyrimidin-2-yl)amino)pyrrolidine-1-carboxylate (500 mg, 1.03 mmol) in HCl/dioxane (10 mL) was stirred at room temperature for 1 hour. The mixture was concentrated under reduced pressure. The residue was quenched with sat. NaHCO₃ (20 mL) and extracted with DCM (30 mL×4). The combined organic layers were washed with brine (50 mL) and dried over Na₂SO₄, filtered, concentrated to give 6-(2,4-difluorophenoxy)-N-(pyrrolidin-3-yl)-8,9-dihydroimidazo[1′,2′:1,6]pyrido[2,3-d]pyrimidin-2-amine (330 mg, 83% yield). LCMS (M+H⁺) m/z: 385.2

### Step 3: Synthesis of 1-(3-((6-(2,4-Difluorophenoxy)-8,9-dihydroimidazo[1′,2′:1,6]pyrido[2,3-d]pyrimidin-2-yl)amino)pyrrolidin-1-yl)prop-2-en-1-one

Acryloyl chloride (78 mg, 0.85 mmol) was added to the solution of 6-(2,4-difluorophenoxy)-N-(pyrrolidin-3-yl)-8,9-dihydroimidazo[1′,2′:1,6]pyrido[2,3-d]pyrimidin-2-amine (330 mg, 0.85 mmol), TEA (260 mg, 2.57 mmol) in DCM (5 mL). The solution was stirred at room temperature for 1 hour. The mixture was quenched with sat NaHCO₃ (20 mL) and extracted with DCM (20 mL×3). The combined organic layers were washed with brine (40 mL), dried over Na₂SO₄, filtered, concentrated to give the crude product, which was purified by Prep-HPLC (0.1% NH₃·H₂O) to give 1-(3-((6-(2,4-difluorophenoxy)-8,9-dihydroimidazo[1′,2′:1,6]pyrido[2,3-d]pyrimidin-2-yl)amino)pyrrolidin-1-yl)prop-2-en-1-one (59 mg, 15.7% yield) as a white solid. ¹H NMR (400 MHz, DMSO-d₆): δ 8.15 (s, 1H), 7.66-7.65 (m, 1H), 7.48-7.43 (m, 1H), 7.32-7.26 (m, 1H), 7.09 (t, J=8.0 Hz, 1H), 6.77 (s, 1H), 6.62-6.49 (m, 1H), 6.15-6.09 (m, 1H), 5.68-5.62 (m, 1H), 4.45-4.36 (m, 1H), 4.00-3.94 (m, 4H), 3.76-3.71 (m, 1H), 3.64-3.54 (m, 2H), 3.48-3.41 (m, 1H), 2.10-2.09 (m, 1H), 2.00-1.91 (m, 1H). LCMS (M+H⁺) m/z: 439.3.

### Example 308: Preparation of 1-(3-((6-(2,4-difluorophenoxy)-8,9-dihydroimidazo[1′,2′:1,6]pyrido[2,3-d]pyrimidin-2-yl)amino)piperidin-1-yl)prop-2-en-1-one (Compound 308)

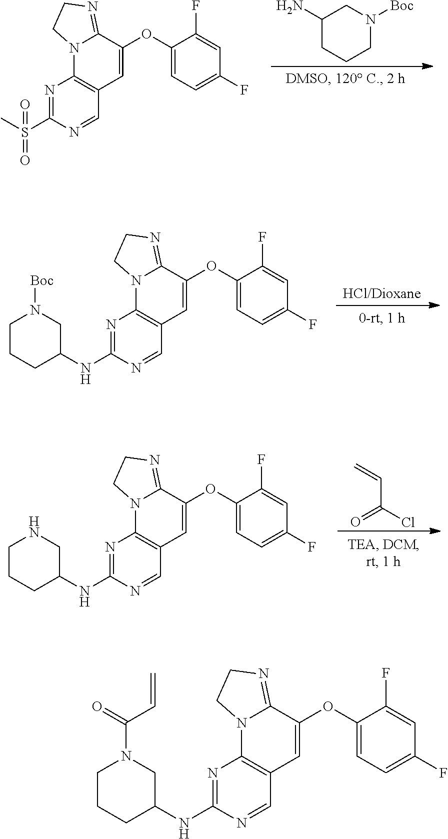

### Step 1: Synthesis of tert-butyl 3-((6-(2,4-difluorophenoxy)-8,9-dihydroimidazo[1′,2′:1,6]pyrido[2,3-d]pyrimidin-2-yl)amino)piperidine-1-carboxylate

tert-butyl 3-aminopiperidine-1-carboxylate (140 mg, 0.70 mmol) was added to a solution of 6-(2,4-difluorophenoxy)-2-(methylsulfonyl)-8,9-dihydroimidazo[1′,2′:1,6]pyrido[2,3-d]pyrimidine (220 mg, 0.58 mmol) in DMSO (6 mL). The reaction mixture was stirred at 120° C. for 2 hours. The mixture was quenched with water (30 mL) and extracted with EA (20 mL×2). The combined organic layers was washed with brine (50 mL) and dried over Na₂SO₄, filtered, concentrated to give tert-butyl 3-((6-(2,4-difluorophenoxy)-8,9-dihydroimidazo[1′,2′:1,6]pyrido [2,3-d]pyrimidin-2-yl)amino)piperidine-1-carboxylate (350 mg, crude) LCMS (M+H⁺) m/z: 499.0

### Step 2: Synthesis of 6-(2,4-Difluorophenoxy)-N-(piperidin-3-yl)-8,9-dihydroimidazo[1′,2′:1,6]pyrido[2,3-d]pyrimidin-2-amine

The solution of tert-butyl 3-((6-(2,4-difluorophenoxy)-8,9-dihydroimidazo [1′,2′:1,6]pyrido[2,3-d]pyrimidin-2-yl)amino)piperidine-1-carboxylate (270 mg, crude) in HCl/dioxane (5 mL) was stirred at room temperature for 1 hour. The mixture was concentrated under reduced pressure. The residue was quenched with sat NaHCO₃ (20 mL) and extracted with DCM (20 mL×2). The combined organic layer was washed with brine (20 mL) and concentrated to give the crude, which was purified by Prep-HPLC (acetonitrile/H₂O=0% to 50%) to give 6-(2,4-Difluorophenoxy)-N-(piperidin-3-yl)-8,9-dihydroimidazo[1′,2′:1,6]pyrido[2,3-d]pyrimidin-2-amine (120 mg, 56% yield). LCMS (M+H+) m/z: 399.2

### Step 3: Synthesis of 1-(3-((6-(2,4-Difluorophenoxy)-8,9-dihydroimidazo[1′,2′:1,6]pyrido[2,3-d]pyrimidin-2-yl)amino)piperidin-1-yl)prop-2-en-1-one

Acryloyl chloride (27 mg, 0.30 mmol) was added to the solution of 6-(2,4-difluorophenoxy)-N-(piperidin-3-yl)-8,9-dihydroimidazo[1′,2′:1,6]pyrido[2,3-d]pyrimidin-2-amine (120 mg, 0.30 mmol), TEA (91 mg, 0.90 mmol) in DCM (5 mL). The solution was stirred at room temperature for 1 hour. The mixture was quenched with sat. NaHCO₃ (20 mL) and extracted with DCM (20 mL×3). The combined organic layers was washed with brine (50 mL) and concentrated to give the crude product, which was purified by Prep-HPLC (0.1% ammonia) to give 1-(3-((6-(2,4-difluorophenoxy)-8,9-dihydroimidazo[1′,2′:1,6]pyrido[2,3-d]pyrimidin-2-yl)amino)piperidin-1-yl)prop-2-en-1-one (16.53 mg, 12.2% yield) as an off-white solid. ¹H NMR (400 MHz, DMSO-d₆): δ 8.13 (s, 1H), 7.48-7.26 (m, 3H), 7.09 (t, J=7.6 Hz, 1H), 6.84-6.77 (m, 2H), 6.10-6.06 (m, 1H), 5.67-5.61 (m, 1H), 4.17-4.13 (m, 1H), 3.98-3.47 (m, 6H), 3.06-2.81 (m, 1H), 2.69-2.52 (m, 1H), 1.94-1.92 (m, 1H), 1.79-1.76 (m, 1H), 1.64-1.53 (m, 1H), 1.42-1.39 (m, 1H). LCMS (M+H⁺) m/z: 453.0.

### Example 309: Preparation of N-methyl-6-(2-methyl-4-((4-phenylphthalazin-1-yl)amino)phenyl)-8,9-dihydroimidazo[1′,2′:1,6]pyrido[2,3-d]pyrimidin-2-amine

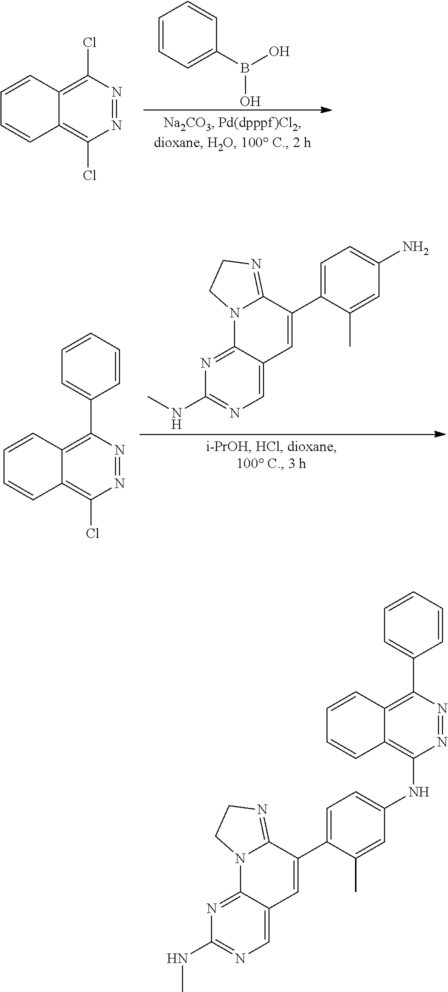

### Step 1: Synthesis of 1-chloro-4-phenylphthalazine

To a solution of 1,4-dichlorophthalazine (652 mg, 3.280 mmol), phenylboronic acid (200 mg, 1.640 mmol) and Na₂CO₃ (521 mg, 4.920 mmol) in dioxane (30 mL) and H₂O (6 mL) was added Pd(dppf)Cl₂ (60 mg, 0.082 mmol). The reaction mixture was stirred at 100° C. under N₂ for 1 hour. The crude product was purified by flash chromatography to afford 1-chloro-4-phenylphthalazine (160 mg, 20.2% yield) as a yellow solid. LCMS (M+H⁺) m/z: 241.1.

### Step 2: Synthesis of N-methyl-6-(2-methyl-4-((4-phenylphthalazin-1-yl)amino)phenyl)-8,9-dihydroimidazo[1′,2′:1,6]pyrido[2,3-d]pyrimidin-2-amine

To a solution of 1-chloro-4-phenylphthalazine (190 mg, 0.623 mmol) and 6-(4-amino-2-methylphenyl)-N-methyl-8,9-dihydroimidazo[1′,2′:1,6]pyrido[2,3-d]pyrimidin-2-amine (150 mg, 0.623 mmol) in i-PrOH (6 mL) was added HCl/dioxane (6 drop). The reaction mixture was stirred at 100° C. under N₂ for 3 h. The result solution was extracted with EA (20 mL×3), concentrated. The crude product was purified with Prep-TLC (DCM:MeOH=30:1) and by Prep-HPLC (0.10%/TFA/CH₃CN/H₂O) to afford N-methyl-6-(2-methyl-4-((4-phenylphthalazin-1-yl)amino)phenyl)-8,9-dihydroimidazo[1′,2′:1,6]pyrido[2,3-d]pyrimidin-2-amine (262.81 mg, 82.6% yield) as a yellow solid. ¹H NMR (400 MHz, DMSO-d₆) δ 9.75 (br s, 1H), 8.89 (s, 1H), 8.85 (d, J=8.4 Hz, 1H), 8.50 (q, J=4.8 Hz, 1H), 8.22 (t, J=7.2 Hz, 1H), 8.12-8.08 (m, 2H), 8.00 (d, J=8.0 Hz, 1H), 7.90 (s, 1H), 7.89 (d, J=8.4 Hz 1H), 7.74-7.72 (m, 2H) 7.66-7.64 (m, 3H), 7.35 (d, J=8.4 Hz, 1H), 4.69-4.56 (m, 2H), 4.07-4.02 (m, 2H), 2.97 (d, J=4.8 Hz, 3H), 2.28 (s, 3H). LCMS (M+H+) m/z: 511.0.

### Example 310: Preparation of N-methyl-6-(2-methyl-4-((4-(4-methylthiophen-2-yl) phthalazin-1-yl)amino)phenyl)-8,9-dihydroimidazo[1′,2′:1,6]pyrido[2,3-d]pyrimidin-2-amine (Compound 310)

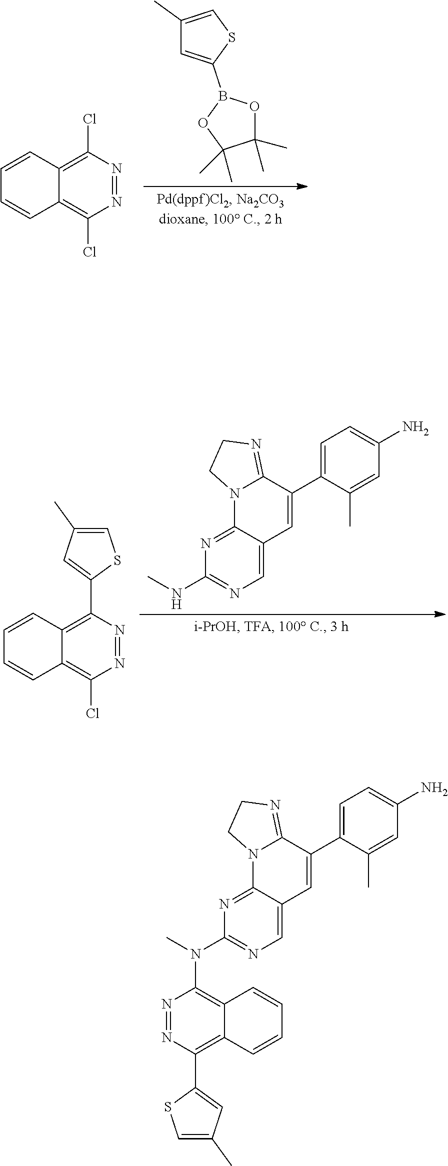

### Step 1: Synthesis of 1-chloro-4-(4-methylthiophen-2-yl) phthalazine

To a solution of 1,4-dichlorophthalazine (800 mg, 4.019 mmol), 4,4,5,5-tetramethyl-2-(4-methylthiophen-2-yl)-1,3,2-dioxaborolane (300 mg, 2.112 mmol) and Na₂CO₃ (672 mg, 6.336 mmol) in dioxane (30 mL) and H₂O (6 mL) was added Pd(dppf)Cl₂ (77 mg, 0.105 mmol). The reaction mixture was stirred at 100° C. under N₂ for 2 hour. The crude product was purified by flash chromatography to afford 1-chloro-4-(4-methylthiophen-2-yl) phthalazine (375 mg, 1.438 mmol, 35.7% yield) as a yellow solid. LCMS (M+H⁺) m/z: 261.1.

### Step 2: Synthesis of N-methyl-6-(2-methyl-4-((4-(4-methylthiophen-2-yl) phthalazin-1-yl)amino)phenyl)-8,9-dihydroimidazo[1′,2′:1,6]pyrido[2,3-d]pyrimidin-2-amine

To a solution of 1-chloro-4-(4-methylthiophen-2-yl) phthalazine (100 mg, 0.326 mmol) and 6-(4-amino-2-methylphenyl)-N-methyl-8,9-dihydroimidazo[1′,2′:1,6]pyrido[2,3-d]pyrimidin-2-amine (127 mg, 0.489 mmol) in i-PrOH (6 mL) was added TFA (1 drop). The reaction mixture was stirred at 100° C. under N₂ for 3 hour. The result solution was extracted with EA (20 mL×3), concentrated. The crude product was purified by prep-TLC (DCM:MeOH=30:1) and by prep-HPLC (0.1%/TFA/CH₃CN/H₂O) to afford N-methyl-6-(2-methyl-4-((4-(4-methylthiophen-2-yl) phthalazin-1-yl)amino)phenyl)-8,9-dihydroimidazo[1′,2′:1,6]pyrido[2,3-d]pyrimidin-2-amine TFA salt (64.5 mg, 30.7% yield) as a yellow solid. ¹H NMR (400 MHz, DMSO-d₆) δ 9.35 (s, 1H), 8.68 (d, J=7.6 Hz, 1H), 8.44-8.42 (m, 2H), 8.14 (s, 1H), 8.10-8.02 (m, 2H), 7.89-7.87 (m, 2H), 7.53 (s, 1H), 7.33 (s, 1H), 7.23 (d, J=8.4 Hz, 1H), 4.29-4.13 (m, 2H), 3.98-3.94 (m, 2H), 2.89 (s, 3H), 2.34 (s, 3H), 2.27 (s, 3H). LCMS (M+H⁺) m/z: 531.1.

### Example 311: Preparation of 1-cyclopropyl-3-(3-fluoro-4-((2-(methylamino)-8,9-dihydroimidazo[1′,2′:1,6]pyrido[2,3-d]pyrimidin-6-yl)oxy)phenyl)urea (Compound 311)

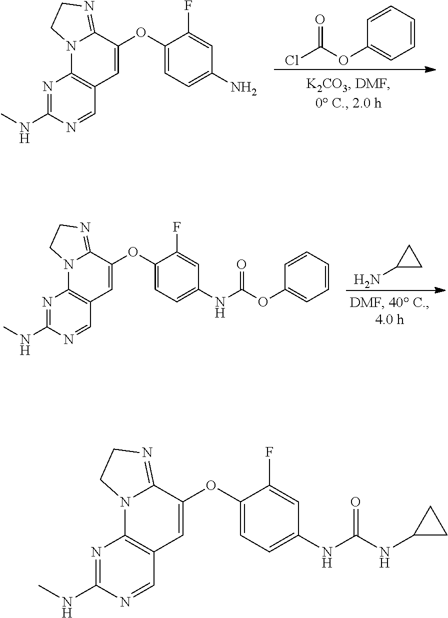

To a mixture of 6-(4-amino-2-fluorophenoxy)-N-methyl-8,9-dihydroimidazo[1′,2′:1,6]pyrido[2,3-d]pyrimidin-2-amine (400 mg, 1.23 mmol) and K₂CO₃ (507 mg, 3.68 mmol) in DMF (10 mL) was added phenyl carbonochloridate (249 mg, 1.59 mmol). The resulting mixture was stirred at 0° C. for 3 h. The reaction mixture was added into water (100 mL), filtered. The collected cake was purified by column chromatography on silica gel (DCM:MeOH=15:1, +0.1% TEA) to afford phenyl (3-fluoro-4-((2-(methylamino)-8,9-dihydroimidazo[1′,2′:1,6]pyrido[2,3-d]pyrimidin-6-yl)oxy)phenyl)carbamate (50 mg, 9.100 yield) as an off-white solid. LCMS (M+H⁺) m/z: 447.2

### Step 2: Synthesis of 1-cyclopropyl-3-(3-fluoro-4-((2-(methylamino)-8,9-dihydroimidazo[1′,2′:1,6]pyrido[2,3-d]pyrimidin-6-yl)oxy)phenyl)urea

To a mixture of phenyl (3-fluoro-4-((2-(methylamino)-8,9-dihydroimidazo[1′,2′:1,6]pyrido[2,3-d]pyrimidin-6-yl)oxy)phenyl)carbamate (50 mg, 0.112 mmol) and cyclopropanamine (25 mg, 0.448 mmol) in DMF (1.5.0 mL) in a sealed tube. The resulting mixture was stirred at 40° C. for 4.0 h. The reaction mixture concentrated to remove cyclopropanamine and purified by Prep-HPLC (0.1% NH₃—H₂O) to give-cyclopropyl-3-(3-fluoro-4-((2-(methylamino)-8,9-dihydroimidazo[1′,2′:1,6]pyrido[2,3-d]pyrimidin-6-yl)oxy)phenyl)urea (16.0 mg, 34.9% yield) as an off-white solid. ¹H NMR (400 MHz, DMSO-d₆): δ 8.54 (s, 1H), 8.10 (s, 1H), 7.61 (dd, J=10.0, 2.4 Hz, 1H), 7.21-7.18 (m, 1H), 7.18-7.13 (m, 2H), 6.53 (s, 1H), 6.47 (d, J=2.4 Hz, 1H), 4.00-3.96 (m, 4H), 2.79 (d, J=4.4 Hz, 3H), 2.56-2.52 (m, 1H), 0.66-0.62 (m, 2H), 0.43-0.39 (m, 2H). LCMS (M+H⁺) m/z: 410.1.

### Example 312: Preparation of 1-cyclopropyl-3-(4-((2-(methylamino)-8,9-dihydroimidazo[1′,2′:1,6]pyrido[2,3-d]pyrimidin-6-yl)oxy)phenyl)urea (Compound 312)

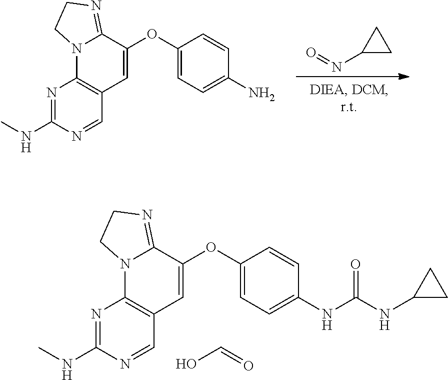

### Step 1: Synthesis of 1-cyclopropyl-3-(4-((2-(methylamino)-8,9-dihydroimidazo[1′,2′:1,6]pyrido[2,3-d]pyrimidin-6-yl)oxy)phenyl)urea

A mixture of 6-(4-aminophenoxy)-N-methyl-8,9-dihydroimidazo[1′,2′:1,6]pyrido[2,3-d]pyrimidin-2-amine (30 mg, 0.1 mmol), isocyanatocyclopropane (27 mg, 0.12 mmol), DIPEA (40 mg, 0.3 mmol) in DCM (3 mL) was stirred for 30 min at 20° C. The reaction was purified by Prep-HPLC (0.1% NH₄HCO₃) to give 1-cyclopropyl-3-(4-((2-(methylamino)-8,9-dihydroimidazo[1′,2′:1,6]pyrido[2,3-d]pyrimidin-6-yl)oxy)phenyl)urea 20.5 mg as yellow solid. ¹H NMR (400 MHz, DMSO-d₆): δ 8.34 (s, 1H), 8.17 (s, 1H), 8.12 (s, 1H), 7.40 (d, J=9.2 Hz, 2H), 7.26 (br, 1H), 6.96 (d, J=9.2 Hz, 2H), 6.66 (br, 1H), 6.40 (d, J=2.4 Hz, 1H), 4.05-3.92 (m, 4H), 2.80 (d, J=4.0 Hz, 3H), 2.54-2.53 (m, 1H), 0.65-0.60 (m, 2H), 0.41-0.37 (m, 2H). LCMS (M+H⁺) m/z: 392.2.

### Example 313: Preparation of 1-(4-chloro-3-(trifluoromethyl)phenyl)-3-(4-((2-(methylamino)-8,9-dihydroimidazo[1′,2′:1,6]pyrido[2,3-d]pyrimidin-6-yl)oxy)phenyl)urea (Compound 313)

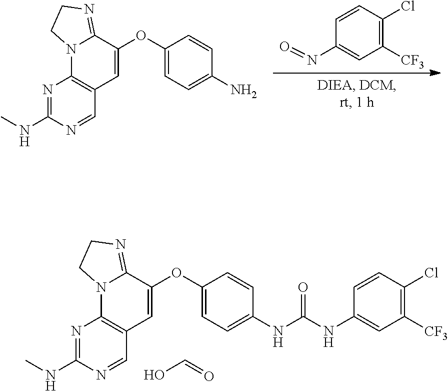

### Step 1: Synthesis of 1-(4-chloro-3-(trifluoromethyl)phenyl)-3-(4-((2-(methylamino)-8,9-dihydroimidazo[1′,2′:1,6]pyrido[2,3-d]pyrimidin-6-yl)oxy)phenyl)urea

To a mixture of 6-(4-aminophenoxy)-N-methyl-8,9-dihydroimidazo[1′,2′:1,6]pyrido[2,3-d]pyrimidin-2-amine (60 mg, 0.2 mmol) in DCM (3 mL), was added 1-chloro-4-isocyanato-2-(trifluoromethyl)benzene (53 mg, 0.24 mmol) and DIPEA (77 mg, 0.6 mmol) at rt. The reaction mixture was stirred for 30 min at 20° C. The reaction was purified by Prep-HPLC to give 1-(4-chloro-3-(trifluoromethyl)phenyl)-3-(4-((2-(methylamino)-8,9-dihydroimidazo[1′,2′:1,6]pyrido[2,3-d]pyrimidin-6-yl)oxy)phenyl)urea 28.8 mg as formic acid salt yellow solid. ¹H NMR (400 MHz, DMSO-d₆): δ 9.46 (s, 1H), 9.15 (s, 1H), 8.19 (s, 2H), 8.11 (d, J=2.4 Hz, 1H), 7.66 (dd, J=9.2, 2.4 Hz, 1H), 7.60 (d, J=8.4 Hz, 1H), 7.47 (d, J=8.4 Hz, 2H), 7.41 (br, 1H), 7.03 (d, J=8.8 Hz, 2H), 6.89 (br, 1H), 4.14-3.95 (m, 4H), 2.81 (s, 3H). LCMS (M+H⁺) m/z: 529.8.

### Example 314: Preparation of 1-(4-chloro-3-(trifluoromethyl)phenyl)-3-(3-fluoro-4-((2-(methylamino)-8,9-dihydroimidazo[1′,2′:1,6]pyrido[2,3-d]pyrimidin-6-yl)oxy)phenyl)urea (Compound 314)

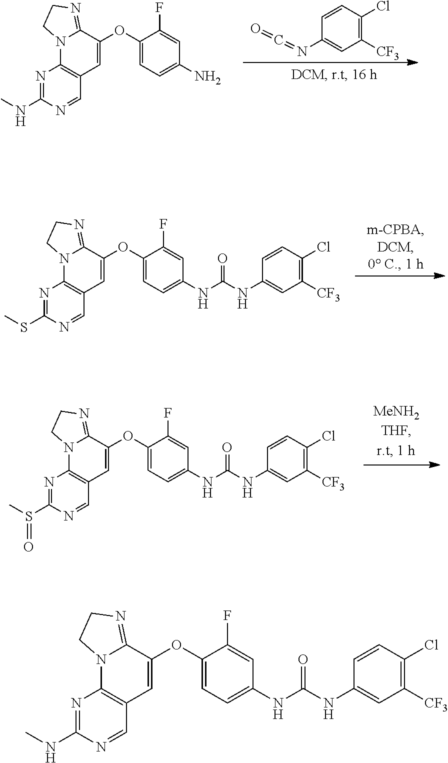 [SMILES: CNc1ncc2c(n1)N1CCN=C1C(Oc1ccc(N)cc1F)=C2 ; O=C=Nc1ccc(Cl)c(C(F)(F)F)c1 ; CSc1ncc2c(n1)N1CCN=C1C(Oc1ccc(NC(=O)Nc3ccc(Cl)c(C(F)(F)F)c3)cc1F)=C2 ; CS(=O)c1ncc2c(n1)N1CCN=C1C(Oc1ccc(NC(=O)Nc3ccc(Cl)c(C(F)(F)F)c3)cc1F)=C2 ; CNc1ncc2c(n1)N1CCN=C1C(Oc1ccc(NC(=O)Nc3ccc(Cl)c(C(F)(F)F)c3)cc1F)=C2]

### Step 1: Synthesis of tert-butyl 1-(4-Chloro-3-(trifluoromethyl)phenyl)-3-(3-fluoro-4-((2-(methylthio)-8,9-dihydroimidazo[1′,2′:1,6]pyrido[2,3-d]pyrimidin-6-yl)oxy)phenyl)urea

To a mixture of 3-Fluoro-4-((2-(methylthio)-8,9-dihydroimidazo[1′,2′:1,6]pyrido[2,3-d]pyrimidin-6-yl)oxy)aniline (100 mg, 0.29 mmol) in DCM (10 mL) was added 1-chloro-4-isocyanato-2-(trifluoromethyl)benzene (77 mg, 0.35 mmol). The resulting mixture was stirred at r.t for 16 hours under N₂. The reaction mixture was quenched with H₂O (20 mL), extracted with DCM (20.0 mL×3). The combined organic phase was washed with brine (50 mL), dried over Na₂SO₄, filtered and concentrated. The residue was purified by column chromatography on silica gel (DCM/MeOH=10/1) to afford 1-(4-chloro-3-(trifluoromethyl)phenyl)-3-(3-fluoro-4-((2-(methylthio)-8,9-dihydroimidazo[1′,2′:1,6]pyrido[2,3-d]pyrimidin-6-yl)oxy)phenyl)urea. (110 mg, 67% yield) as yellow oil. LCMS (M+H⁺) m/z: 565.1.

### Step 2: Synthesis of tert-butyl 6-(3-(2,4-dichlorobenzoyl)-1-(tetrahydro-2H-pyran-2-yl)-1H-pyrazolo[3,4-b]pyridin-5-yl)-3,4-dihydroisoquinoline-2(1H)-carboxylate

To a solution of 1-(4-Chloro-3-(trifluoromethyl)phenyl)-3-(3-fluoro-4-((2-(methylsulfinyl)-8,9-dihydroimidazo[1′,2′:1,6]pyrido[2,3-d]pyrimidin-6-yl)oxy)phenyl)urea (90 mg, 0.16 mmol) in DCM (5.0 mL), was added m-CPBA (64 mg, 0.32 mmol) at 0° C. The mixture was stirred for 1 hour at 0° C. The reaction mixture was concentrated to afford tert-butyl 6-(3-(2,4-dichlorobenzoyl)-1-(tetrahydro-2H-pyran-2-yl)-1H-pyrazolo[3,4-b]pyridin-5-yl)-3,4-dihydroisoquinoline-2(1H)-carboxylate (120 mg, crude) as an off-white solid. LCMS (M+H⁺) m/z: 581.2.

### Step 3: Synthesis of 1-(4-Chloro-3-(trifluoromethyl)phenyl)-3-(3-fluoro-4-((2-(methylamino)-8,9-dihydroimidazo[1′,2′:1,6]pyrido[2,3-d]pyrimidin-6-yl)oxy)phenyl)urea

To a mixture of 1-(4-chloro-3-(trifluoromethyl)phenyl)-3-(3-fluoro-4-((2-(methylsulfinyl)-8,9-dihydroimidazo[1′,2′:1,6]pyrido[2,3-d]pyrimidin-6-yl)oxy)phenyl)urea (120 mg, 0.21 mmol) in THF (2.0 mL), was added MeNH₂ (1 mL, 1M in THF). The mixture was concentrated. The residue was purified by column chromatography on silica gel (DCM/MeOH=10/1) to give crude product, which was further purified by Prep-HPLC (0.1% ammonia) to afford 1-(4-chloro-3-(trifluoromethyl)phenyl)-3-(3-fluoro-4-((2-(methylamino)-8,9-dihydroimidazo[1′,2′:1,6]pyrido[2,3-d]pyrimidin-6-yl)oxy)phenyl)urea (17.7 mg, 15.7% yield) as a white solid. ¹H NMR (400 MHz, DMSO-d₆): δ 9.24 (s, 1H), 9.08 (s, 1H), 8.12-8.10 (m, 2H), 7.69-7.61 (m, 3H), 7.23-7.18 (m, 3H), 6.64 (s, 1H), 4.00-3.96 (m, 4H), 2.81 (d, J=4.4 Hz, 3H). LCMS (M+H⁺) m/z: 548.2.

### Example 315: Preparation of 1-cyclopropyl-3-(2-fluoro-4-methyl-5-(2-(methylamino)-8,9-dihydroimidazo[1′,2′:1,6]pyrido[2,3-d]pyrimidin-6-yl)phenyl)urea (Compound 315)

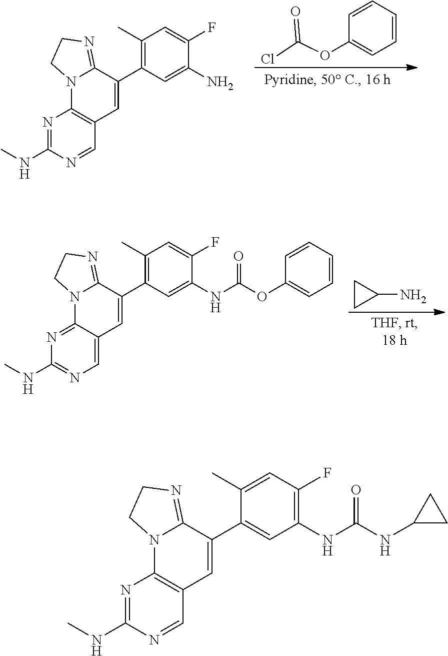

The preparation of 6-(5-amino-4-fluoro-2-methylphenyl)-N-methyl-8,9-dihydroimidazo[1′,2′:1,6]pyrido[2,3-d]pyrimidin-2-amine was described in Example 139

### Step 1: Synthesis of phenyl (2-fluoro-4-methyl-5-(2-(methylamino)-8,9-dihydroimidazo[1′,2′:1,6]pyrido[2,3-d]pyrimidin-6-yl)phenyl)carbamate

To a solution of 6-(5-amino-4-fluoro-2-methylphenyl)-N-methyl-8,9-dihydroimidazo[1′,2′:1,6]pyrido[2,3-d]pyrimidin-2-amine (80 mg, 0.247 mmol) in pyridine (2 mL) was added phenyl carbonochloridate (386 mg, 2.469 mmol). The reaction mixture was stirred at 50° C. for 16 h. Concentration in vacuum afforded phenyl (2-fluoro-4-methyl-5-(2-(methylamino)-8,9-dihydroimidazo[1′,2′:1,6]pyrido[2,3-d]pyrimidin-6-yl)phenyl)carbamate (80 mg, crude), which was used for next step directly.

### Step 2: Synthesis of 1-cyclopropyl-3-(2-fluoro-4-methyl-5-(2-(methylamino)-8,9-dihydroimidazo[1′,2′:1,6]pyrido[2,3-d]pyrimidin-6-yl)phenyl)urea

To a solution of phenyl (2-fluoro-4-methyl-5-(2-(methylamino)-8,9-dihydroimidazo[1′,2′:1,6]pyrido[2,3-d]pyrimidin-6-yl)phenyl)carbamate (80 mg, 0.180 mmol) in pyridine (3 mL) was added cyclopropylamine (31 mg, 0.541 mmol). The reaction mixture was stirred rt for 16 h. LCMS show the reaction was ok. The resulting mixture was purified by prep-HPLC (0.1% TFA) to afford 1-cyclopropyl-3-(2-fluoro-4-methyl-5-(2-(methylamino)-8,9-dihydroimidazo[1′,2′:1,6]pyrido[2,3-d]pyrimidin-6-yl)phenyl)urea (6.1 mg, 13.2%) as a yellow solid. ¹H NMR (400 MHz, CD₃OD): δ 8.77 (s, 1H), 8.00 (s, 1H), 7.89 (d, J=8.0 Hz, 1H), 7.17 (d, J=12.0 Hz, 1H), 4.77 (t, J=10.0 Hz, 2H), 4.12 (t, J=10.0 Hz, 2H), 3.07 (s, 3H), 2.59-2.57 (m, 1H), 2.21 (s, 3H), 0.75-0.73 (m, 2H), 0.52-0.48 (m, 2H). LCMS (M+H⁺) m/z: 408.1.

### Example 316: Preparation of 1-(2-fluoro-4-methyl-5-(2-(methylamino)-8,9-dihydroimidazo[1′,2′:1,6]pyrido[2,3-d]pyrimidin-6-yl)phenyl)-3-(3,3,3-trifluoropropyl)urea (Compound 316)

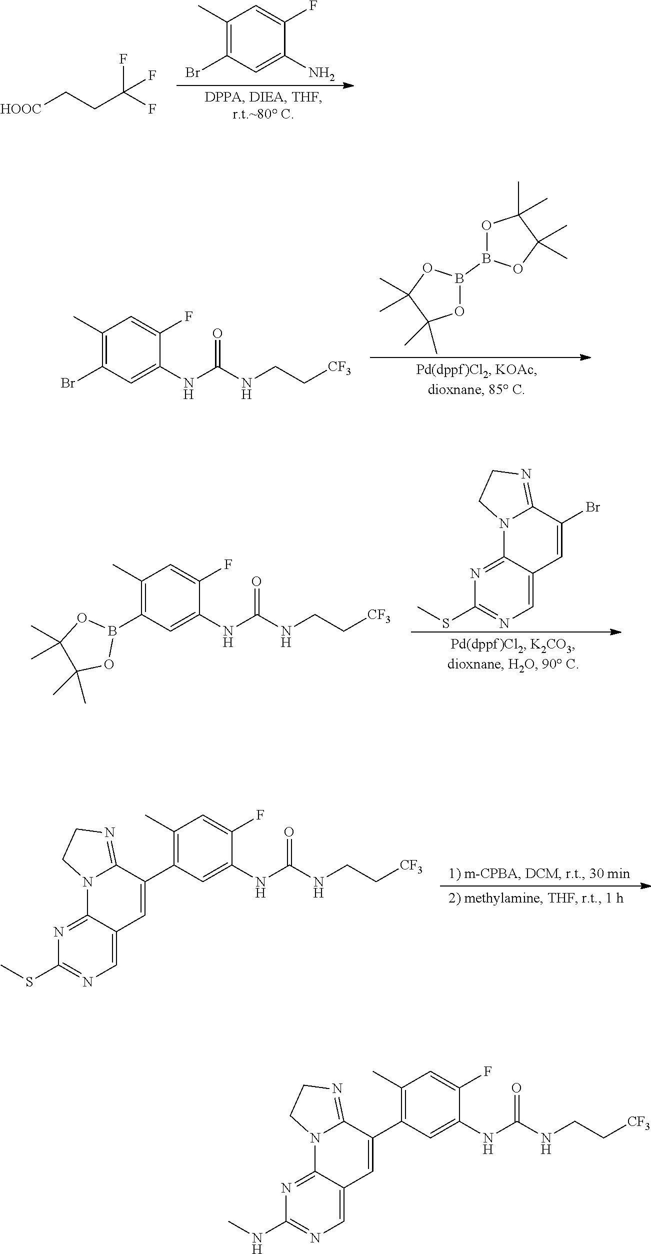

### Step 1: Synthesis of 1-(5-bromo-2-fluoro-4-methylphenyl)-3-(3,3,3-trifluoropropyl)urea

To a solution of 4,4,4-trifluorobutanoic acid (540 mg, 3.8 mmol) in THF (8 mL) was added DPPA (1.05 g, 3.8 mmol) and DIEA (980 mg, 7.6 mmol), the reaction mixture was stirred at r.t. for 30 min. Then 5-bromo-2-fluoro-4-methylaniline (775 mg, 3.8 mmol) was added, the reaction mixture was stirred at 80° C. for 4 h. LCMS showed the reaction completed. The reaction mixture was diluted to water (20 mL), extracted by EA (20 mL×3). The combined organic phase was washed by brine (50 mL), dried over anhydrous Na₂SO₄, concentrated to obtain a white solid, which was purified by chromatography column (PE:EA=1:1) to afford 1-(5-bromo-2-fluoro-4-methylphenyl)-3-(3,3,3-trifluoropropyl)urea (750 mg, 58% yield). LCMS (M+H⁺) m/z: 342.8

### Step 2: Synthesis of 1-(2-fluoro-4-methyl-5-(4,4,5,5-tetramethyl-1,3,2-dioxaborolan-2-yl)phenyl)-3-(3,3,3-trifluoropropyl)urea

To a solution of 1-(5-bromo-2-fluoro-4-methylphenyl)-3-(3,3,3-trifluoropropyl)urea (750 mg, 2.19 mmol) in dioxane (10 mL) was added Pin2B2 (557 mg, 2.19 mmol), KOAc (358 mg, 4.38 mmol) and Pd(dppf)Cl₂ (153 mg, 0.22 mmol) under nitrogen atmosphere. The mixture was stirred at 85° C. for 2 h. LCMS showed the reaction completed. The mixture was diluted to water (10 mL), extracted by EA (10 mL*3), combined organic phase was washed by brine (20 mL), dried over anhydrous Na₂SO₄, filtered and concentrated to afford a crude solid, which was purified by chromatography column (PE:EA=2:1) to afford 1-(2-fluoro-4-methyl-5-(4,4,5,5-tetramethyl-1,3,2-dioxaborolan-2-yl)phenyl)-3-(3,3,3-trifluoropropyl)urea (510 mg, 60% yield) as white solid. LCMS (M+H⁺) m/z: 391.2

### Step 3: Synthesis of 1-(2-fluoro-4-methyl-5-(2-(methylthio)-8,9-dihydroimidazo[1′,2′:1,6]pyrido[2,3-d]pyrimidin-6-yl)phenyl)-3-(3,3,3-trifluoropropyl)urea

To a solution of 1-(2-fluoro-4-methyl-5-(4,4,5,5-tetramethyl-1,3,2-dioxaborolan-2-yl)phenyl)-3-(3,3,3-trifluoropropyl)urea (200 mg, 0.51 mmol) in dioxane (4 mL) and H₂O (1 mL) was added 6-bromo-2-(methylthio)-8,9-dihydroimidazo[1′,2′:1,6]pyrido[2,3-d]pyrimidine (151 mg, 0.51 mmol), K₂CO₃ (142 mg, 1.02 mmol) and Pd(dppf)Cl₂ (37 mg, 0.05 mmol) under nitrogen atmosphere. The mixture was stirred at 80° C. for 3 h. LCMS showed the reaction completed. The mixture was diluted to water (10 mL), extracted by EA (10 mL×3). The combined organic phase was washed by brine (20 mL), dried over anhydrous Na₂SO₄, filtered and concentrated to afford a crude solid, which was purified by chromatography column (PE:EA=1:2) to afford 1-(2-fluoro-4-methyl-5-(2-(methylthio)-8,9-dihydroimidazo[1′,2′:1,6]pyrido[2,3-d]pyrimidin-6-yl)phenyl)-3-(3,3,3-trifluoropropyl)urea (80 mg, 33% yield) as yellow solid. LCMS (M+H+) m/z: 480.9

### Step 4: Synthesis of 1-(2-fluoro-4-methyl-5-(2-(methylamino)-8,9-dihydroimidazo[1′,2′:1,6]pyrido[2,3-d]pyrimidin-6-yl)phenyl)-3-(3,3,3-trifluoropropyl)urea

To a solution of 6-bromo-2-(methylthio)-8,9-dihydroimidazo[1′,2′:1,6]pyrido[2,3-d]pyrimidine (80 mg, 0.17 mmol) in DCM (5 mL) was added m-CPBA (59 mg, 0.34 mmol), the reaction mixture was stirred at r.t. for 30 min. LCMS showed the reaction completed. The reaction mixture was concentrated to obtain a yellow solid. The crude solid in THF (3 mL) was added methylamine in THF (1 mL, 2 mmol). The mixture was stirred at r.t. for 1 h. LCMS showed the reaction completed. The mixture was diluted with water (10 mL), extracted by EA (10 mL×3). The combined organic phase was washed by brine (20 mL), dried over anhydrous Na₂SO₄, filtered and concentrated to afford a crude solid, which was purified by prep-HPLC to afford 1-(2-fluoro-4-methyl-5-(2-(methylamino)-8,9-dihydroimidazo[1′,2′:1,6]pyrido[2,3-d]pyrimidin-6-yl)phenyl)-3-(3,3,3-trifluoropropyl)urea (38.2 mg) as yellow solid. ¹H NMR (400 MHz, DMSO-d₆): δ 8.41 (s, 1H), 8.25 (s, 1H), 8.18 (s, 1H), 7.92 (d, J=8.8 Hz, 1H), 7.52-7.43 (m, 1H), 7.16-7.12 (m, 1H), 7.07 (d, J=12.4 Hz, 1H), 6.73 (t, J=6.0 Hz, 1H), 4.10-3.94 (m, 2H), 3.90 (t, J=9.2 Hz, 2H), 3.32-3.28 (m, 2H), 2.84 (s, 3H), 2.42-2.38 (m, 2H), 2.13 (s, 3H). LCMS (M+H⁺) m/z: 464.0.

### Example 317: Preparation of 1-(3,3-dimethylbutyl)-3-(2-fluoro-4-methyl-5-(2-(methylamino)-8,9-dihydroimidazo[1′,2′:1,6]pyrido[2,3-d]pyrimidin-6-yl)phenyl)urea (Compound 317)

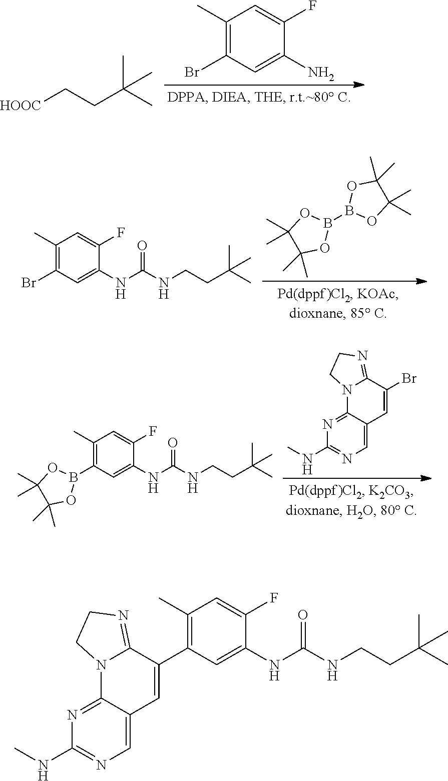

### Step 1: Synthesis of 1-(5-bromo-2-fluoro-4-methylphenyl)-3-(3,3-dimethylbutyl)urea

To a solution of 4,4-dimethylpentanoic acid (500 mg, 3.8 mmol) in THF (8 mL) was added DPPA (1.05 g, 3.8 mmol) and DIEA (980 mg, 7.6 mmol), the reaction mixture was stirred at r.t. for 30 min. Then 5-bromo-2-fluoro-4-methylaniline (775 mg, 3.8 mmol) was added, the reaction mixture was stirred at 80° C. for 4 h. LCMS showed the reaction completed. The reaction mixture was diluted to water (20 mL), extracted by EA (20 mL×3). The combined organic phase was washed by brine (50 mL), dried over anhydrous Na₂SO₄, concentrated to obtain a white solid, which was purified by chromatography column (PE:EA=1:1) to afford 1-(5-bromo-2-fluoro-4-methylphenyl)-3-(3,3-dimethylbutyl)urea (220 mg, 30% yield). LCMS (M+H⁺) m/z: 333.0

### Step 2: Synthesis of 1-(3,3-dimethylbutyl)-3-(2-fluoro-4-methyl-5-(4,4,5,5-tetramethyl-1,3,2-dioxaborolan-2-yl)phenyl)urea

To a solution of 1-(5-bromo-2-fluoro-4-methylphenyl)-3-(3,3-dimethylbutyl)urea (220 mg, 0.66 mmol) in dioxane (5 mL) was added Pin2B2 (201 mg, 0.79 mmol), KOAc (129 mg, 1.32 mmol) and Pd(dppf)Cl2 (51 mg, 0.07 mmol) under nitrogen atmosphere. The mixture was stirred at 85° C. for 2 h. LCMS showed the reaction completed. The mixture was diluted to water (10 mL), extracted by EA (10 mL×3). The combined organic phase was washed by brine (20 mL), dried over anhydrous Na₂SO₄, filtered and concentrated to afford a crude solid, which was purified by chromatography column (PE:EA=2:1) to afford 1-(3,3-dimethylbutyl)-3-(2-fluoro-4-methyl-5-(4,4,5,5-tetramethyl-1,3,2-dioxaborolan-2-yl)phenyl)urea (80 mg, 32% yield) as white solid. LCMS (M+H⁺) m/z: 379.0

### Step 3: Synthesis of 1-(3,3-dimethylbutyl)-3-(2-fluoro-4-methyl-5-(2-(methylamino)-8,9-dihydroimidazo[1′,2′:1,6]pyrido[2,3-d]pyrimidin-6-yl)phenyl)urea

To a solution of 1-(3,3-dimethylbutyl)-3-(2-fluoro-4-methyl-5-(4,4,5,5-tetramethyl-1,3,2-dioxaborolan-2-yl)phenyl)urea (80 mg, 0.21 mmol) in dioxane (4 mL) and H₂O (1 mL) was added 6-bromo-N-methyl-8,9-dihydroimidazo[1′,2′:1,6]pyrido[2,3-d]pyrimidin-2-amine (58 mg, 0.21 mmol), K2CO3 (58 mg, 0.42 mmol) and Pd(dppf)Cl₂ (15 mg, 0.02 mmol) under nitrogen atmosphere. The mixture was stirred at 80° C. for 3 h. LCMS showed the reaction completed. The mixture was diluted to water (10 mL), extracted by EA (10 mL×3). The combined organic phase was washed by brine (20 mL), dried over anhydrous Na₂SO₄, filtered and concentrated to afford a crude solid, which was purified by Prep-HPLC to afford 1-(3,3-dimethylbutyl)-3-(2-fluoro-4-methyl-5-(2-(methylamino)-8,9-dihydroimidazo[1′,2′:1,6]pyrido[2,3-d]pyrimidin-6-yl)phenyl)urea (10.1 mg) as yellow solid. ¹H NMR (400 MHz, DMSO-d₆): δ 8.28 (s, 1H), 8.19 (s, 1H), 8.17 (s, 1H), 7.96 (d, J=8.8 Hz, 1H), 7.53-7.48 (m, 1H), 7.22-7.15 (m, 1H), 7.06 (d, J=12.0 Hz, 1H), 6.46 (t, J=5.2 Hz, 1H), 4.13-4.00 (m, 2H), 3.90 (t, J=10.0 Hz, 2H), 3.10-3.04 (m, 2H), 2.85 (s, 3H), 2.12 (s, 3H), 1.38-1.27 (m, 2H), 0.88 (s, 9H). LCMS (M+H⁺) m/z: 452.0.

### Example 318: Preparation of 1-(3,3-dimethylbutyl)-3-(2-fluoro-4-methyl-5-((2-(methylamino)-8,9-dihydroimidazo[1′,2′:1,6]pyrido[2,3-d]pyrimidin-6-yl)amino)phenyl)urea (Compound 318)

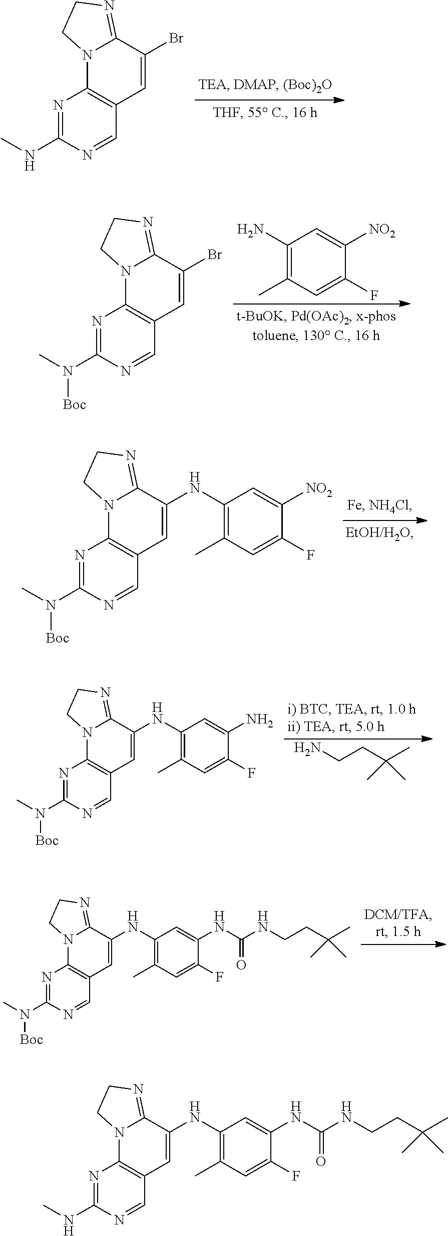

### Step 1: Synthesis of tert-butyl (6-bromo-8,9-dihydroimidazo[1′,2′:1,6]pyrido[2,3-d]pyrimidin-2-yl)(methyl) carbamate

A mixture of 6-bromo-N-methyl-8,9-dihydroimidazo[1′,2′:1,6]pyrido[2,3-d]pyrimidin-2-amine (500 mg, 1.79 mmol), (Boc)₂O (1.54 g, 7.14 mmol) and DMAP (22 mg, 0.179 mmol) in THF (20.0 mL) was stirred at 55° C. for 16 h. The reaction mixture was concentrated. The residue was purified by column chromatography on silica gel (DCM:MeOH=20:1) to afford tert-butyl (6-bromo-8,9-dihydroimidazo[1′,2′:1,6]pyrido[2,3-d]pyrimidin-2-yl)(methyl) carbamate (587 mg, 86.5% yield) as brown oil. LCMS (M+H⁺) m/z: 380.0 and 382.0.

### Step 2: Synthesis of tert-butyl (6-((4-fluoro-2-methyl-5-nitrophenyl)amino)-8,9-dihydroimidazo[1′,2′:1,6]pyrido[2,3-d]pyrimidin-2-yl)(methyl)carbamate

A mixture of tert-butyl (6-bromo-8,9-dihydroimidazo[1′,2′:1,6]pyrido[2,3-d]pyrimidin-2-yl)(methyl)carbamate (200 mg, 0.53 mmol) and 4-fluoro-2-methyl-5-nitroaniline (134 mg, 0.79 mmol), Pd(OAc)₂ (24 mg, 0.11 mmol), x-phos (125 mg, 0.26 mmol) and t-BuOK (177 mg, 1.58 mmol) in toluene (5.0 mL) was stirred at 130° C. for 16 h under N₂. The reaction mixture was concentrated and the residue was purified by column chromatography on silica gel (PE/EA=2/1) and followed by Prep-HPLC (ACN %=46%, 0.1% HCl) to afford crude tert-butyl (6-((4-fluoro-2-methyl-5-nitrophenyl)amino)-8,9-dihydroimidazo[1′,2′:1,6]pyrido[2,3-d]pyrimidin-2-yl)(methyl)carbamate (70 mg, 28.3% yield) as a yellow solid. LCMS (M+H⁺) m/z: 470.3

### Step 3: Synthesis of tert-butyl (6-((5-amino-4-fluoro-2-methylphenyl)amino)-8,9-dihydroimidazo[1′,2′:1,6]pyrido[2,3-d]pyrimidin-2-yl)(methyl)carbamate

To a mixture of tert-butyl (6-((4-fluoro-2-methyl-5-nitrophenyl)amino)-8,9-dihydroimidazo[1′,2′:1,6]pyrido[2,3-d]pyrimidin-2-yl)(methyl)carbamate (70 mg, 0.15 mmol) and NH₄Cl (81 mg, 1.5 mmol) in THF (3.0 mL) and H₂O (3.0 mL) was added Fe (84 mg, 1.5 mmol). The mixture was stirred at 80° C. for 3.0 h under N₂, then diluted with water (20.0 mL), extracted with EA (30 mL×3). The combined organic phase was washed with brine, dried over Na₂SO₄, filtered and concentrated to afford tert-butyl (6-((5-amino-4-fluoro-2-methylphenyl)amino)-8,9-dihydroimidazo[1′,2′:1,6]pyrido[2,3-d]pyrimidin-2-yl)(methyl)carbamate (50 mg, 76.3% yield) as a brown solid. LCMS (M+H⁺) m/z: 440.3.

### Step 4: Synthesis of tert-butyl (6-((5-(3-(3,3-dimethylbutyl)ureido)-4-fluoro-2-methylphenyl)amino)-8,9-dihydroimidazo[1′,2′:1,6]pyrido[2,3-d]pyrimidin-2-yl)(methyl)carbamate

To a mixture of tert-butyl (6-((5-amino-4-fluoro-2-methylphenyl)amino)-8,9-dihydroimidazo[1′,2′:1,6]pyrido[2,3-d]pyrimidin-2-yl)(methyl)carbamate (15 mg, 0.034 mmol) and Et₃N (10.3 mg, 0.102 mmol) in THF (1.5 mL) was added triphosgene (5.0 mg, 0.017 mmol) and the mixture was stirred at r.t for 1.0 h. A solution of 3,3-dimethylbutan-1-amine (7.0 mg, 0.068 mmol) in THF (0.5 mL) was added drop-ise. The resulting mixture was stirred at r.t. for 5.0 h under N₂. After the reaction was completed, the reaction mixture was diluted with water (20 mL), extracted with DCM (20 mL×3). The combined organic phase was washed with brine dried over Na₂SO₄, filtered and concentrated to afford crude tert-butyl (6-((5-(3-(3,3-dimethylbutyl)ureido)-4-fluoro-2-methylphenyl)amino)-8,9-dihydroimidazo[1′,2′:1,6]pyrido[2,3-d]pyrimidin-2-yl)(methyl)carbamate (15 mg, 78.9% yield) as brown oil. LCMS (M-Boc+H⁺) m/z: 467.4.

### Step 5: Synthesis of 1-(3,3-dimethylbutyl)-3-(2-fluoro-4-methyl-5-((2-(methylamino)-8,9-dihydroimidazo[1′,2′:1,6]pyrido[2,3-d]pyrimidin-6-yl)amino)phenyl)urea hydrogen chloride

To a solution of tert-butyl (6-((5-(3-(3,3-dimethylbutyl)ureido)-4-fluoro-2-methylphenyl)amino)-8,9-dihydroimidazo[1′,2′:1,6]pyrido[2,3-d]pyrimidin-2-yl)(methyl)carbamate (15 mg) in DCM (2.0 mL) was added TFA (1.0 mL). The resulting mixture was stirred at r.t. for 1.5 h, concentrated. The residue was purified by Prep-HPLC (0.1% NH₃·H₂O), then by Prep-HPLC (0.1% HCl) to afford 1-(3,3-dimethylbutyl)-3-(2-fluoro-4-methyl-5-((2-(methylamino)-8,9-dihydroimidazo[1′,2′:1,6]pyrido[2,3-d]pyrimidin-6-yl)amino)phenyl)urea hydrogen chloride (1.87 mg, 15.1% yield) as a yellow solid. ¹H NMR (400 MHz, CD₃OD): δ 8.73 (s, 1H), 7.72 (s, 1H), 7.34 (d, J=8.0 Hz, 1H), 7.02 (d, J=11.6 Hz, 1H), 4.78-4.76 (m, 2H), 4.25 (t, J=10.0 Hz, 2H), 3.22-3.17 (m, 2H), 3.06 (s, 3H), 2.20 (s, 3H), 1.45-1.41 (m, 2H), 0.94 (s, 9H). LCMS (M+H⁺) m/z: 467.3.

### Example 319: Preparation of 1-(4-chloro-2-fluoro-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea

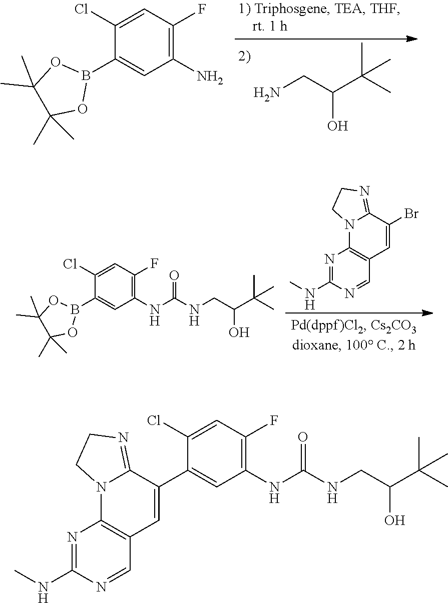

### Step 1: Synthesis of a mixture of 1-(4-chloro-2-fluoro-5-(4,4,5,5-tetramethyl-1,3,2-dioxaborolan-2-yl)phenyl)-3-(2-hydroxy-3,3-dimethylbutyl)urea

To a solution of 1-amino-3,3-dimethylbutan-2-ol (51 mg, 0.44 mmol) and TEA (133 mg, 1.32 mmol) in THF (2 mL) was added triphosgene (32 mg, 0.11 mmol). The solution stirred at r.t under N2 for 1 h. Then the reaction solution was added to a solution of 4-chloro-2-fluoro-5-(4,4,5,5-tetramethyl-1,3,2-dioxaborolan-2-yl)aniline (60 mg, 0.22 mmol) in THF (2 mL). The mixture was stirred at r.t under N2 for 1 h. The mixture was filtered. The filtrate was diluted with DCM/MeOH (10/1, 100 mL) and washed with water (20 mL). The organic phase was concentrated to afford 1-(4-chloro-2-fluoro-5-(4,4,5,5-tetramethyl-1,3,2-dioxaborolan-2-yl)phenyl)-3-(2-hydroxy-3,3-dimethylbutyl)urea (92 mg crude) as a yellow oil. LCMS (M+H⁺) m/z: 333.1 and 415.3.

### Step 2: Synthesis of 1-(4-chloro-2-fluoro-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea hydrochloride

A mixture of 1-(4-chloro-2-fluoro-5-(4,4,5,5-tetramethyl-1,3,2-dioxaborolan-2-yl)phenyl)-3-(2-hydroxy-3,3-dimethylbutyl)urea

(90 mg crude, 0.22 mmol), 6-bromo-N-methyl-8,9-dihydroimidazo[1′,2′:1,6]pyrido[2,3-d]pyrimidin-2-amine (62 mg, 0.22 mmol), Cs₂CO₃ (215 mg, 0.66 mmol) and Pd(dppf)Cl₂ (16 mg, 0.022 mmol) in dioxane (6 mL) and water (1.5 mL) was stirred at 100° C. under N₂ for 1 h. The reaction mixture was cooled to r.t. and purified by flash chromatography to get crude product 40 mg, which was purified by prep-HPLC (0.1%/HCl/CH₃CN/H₂O) to afford 1-(4-chloro-2-fluoro-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea hydrochloride (14.9 mg, 13% yield) as a yellow solid. ¹H NMR (400 MHz, DMSO-d6) δ 9.93 (s, 1H), 8.88 (s, 2H), 8.59-8.46 (m, 1H), 8.35 (d, J=7.2 Hz, 1H), 8.17 (s, 1H), 7.64 (d, J=11.2 Hz, 1H), 6.94 (s, 1H), 4.69-4.53 (m, 2H), 4.12-3.97 (m, 2H), 3.46-3.43 (m, 1H), 3.08 (d, J=8.4 Hz, 1H), 2.95 (d, J=4.8 Hz, 3H), 2.51-2.49 (m, 1H), 0.88 (s, 9H). LCMS (M+H⁺) m/z: 488.2.

### Example 320: Preparation of 1-(6-(5-amino-2-chloro-4-fluorophenyl)-8,9-dihydroimidazo[1′,2′:1,6]pyrido[2,3-d]pyrimidin-2-yl)-3-(2-hydroxy-3,3-dimethylbutyl)-1-methylurea (Compound 320)

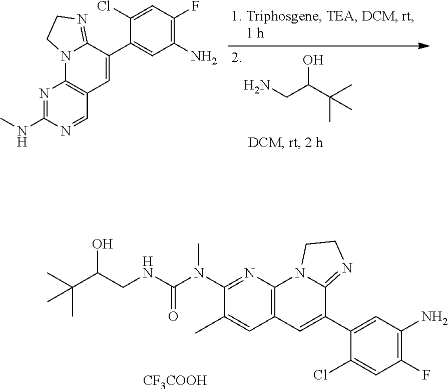

### Step 1: Synthesis of 1-(4-chloro-2-fluoro-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea

A mixture of 6-(5-amino-2-chloro-4-fluorophenyl)-N-methyl-8,9-dihydroimidazo[1′,2′:1,6]pyrido[2,3-d]pyrimidin-2-amine (60 mg, 0.16 mmol, 1.0 eq) in THE (10 mL) was added Ttriphosgene (18.4 mg, 0.062 mmol, 0.4 eq) and TEA (47 mg, 0.468 mmol, 3.0 eq). The mixture was stirred at rt under N2 for 1 h. Then 1-amino-3,3-dimethylbutan-2-ol (36 mg, 0.31 mmol, 2.0 eq) was added. The mixture was stirred at rt under N2 for 2 h. The resulting mixture was purified by prep-HPLC (0.1% TFA) to afford 1-(4-chloro-2-fluoro-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea (14.3 mg, 18.4%) as a yellow solid. ¹H NMR (400 MHz, CD₃OD): δ 9.14 (s, 1H), 8.26 (s, 1H), 7.28 (d, J=10.8 Hz, 1H), 6.90 (d, J=9.2 Hz, 1H), 4.94-4.85 (m, 2H), 4.26-4.20 (m, 2H), 3.82 (dd, J=13.2, 2.4 Hz, 1H), 3.64 (s, 3H), 3.40 (dd, J=10.0, 2.4 Hz, 1H), 3.15-3.09 (m, 1H), 0.98 (s, 9H). LCMS (M+H⁺) m/z: 488.0.

### Example 321: Preparation of 1-(2-fluoro-4-methyl-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea (Compound 321)

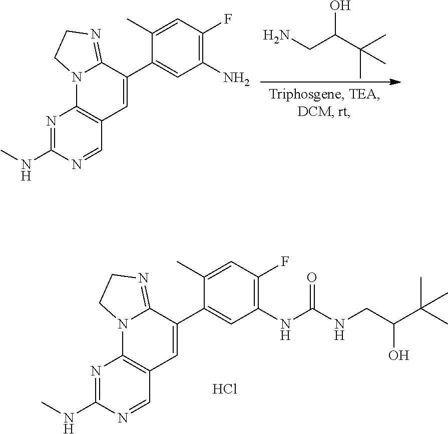

### Step 1: Synthesis of 1-(2-fluoro-4-methyl-5-(2-(methylamino)-8,9-dihydroimidazo [1′,2′:1,6]pyrido[2,3-d]pyrimidin-6-yl)phenyl)-3-(2-hydroxy-3,3-dimethylbutyl)urea

A mixture of 1-amino-3,3-dimethylbutan-2-ol (40 mg, 0.34 mmol, 2.0 eq) and TEA (33 mg, 0.0.51 mmol, 3.0 eq) in THF (15 mL) was added triphosgene (20 mg, 0.07 mmol, 0.4 eq). The mixture was stirred at 0° C. for 1 h. After 1 h, 6-(5-amino-4-fluoro-2-methylphenyl)-N-methyl-8,9-dihydroimidazo[1′,2′:1,6]pyrido[2,3-d]pyrimidin-2-amine (60 mg, 0.17 mmol, 1.0 eq) was added to the solution. The mixture was stirred at rt for 16 h. LCMS show the reaction was OK. The resulting mixture was purified by prep-HPLC (0.1% HCl) to afford 1-(2-fluoro-4-methyl-5-(2-(methylamino)-8,9-dihydroimidazo[1′,2′:1,6]pyrido[2,3-d]pyrimidin-6-yl)phenyl)-3-(2-hydroxy-3,3-dimethylbutyl)urea. (4.7 mg, 5% yield) as a yellow solid. ¹H NMR (400 MHz, CD₃OD): δ 8.87 (s, 1H), 8.05 (s, 1H), 7.96-7.63 (m, 1H), 7.19 (d, J=12.0 Hz, 1H), 4.79-4.76 (m, 2H), 4.17 (t, J=10.0 Hz, 2H), 3.61 (dd, J=13.6, 2.4 Hz, 1H), 3.32-3.28 (m, 1H), 3.12 (s, 3H), 2.98-2.92 (m, 1H), 2.23 (s, 3H), 0.96 (s, 9H). LCMS (M+H⁺) m/z: 468.1.

### Example 322: Preparation of 1-(6-(5-amino-2-bromo-4-fluorophenyl)-8,9-dihydroimidazo[1′,2′:1,6]pyrido[2,3-d]pyrimidin-2-yl)-3-(2-hydroxy-3,3-dimethylbutyl)-1-methylurea (Compound 322)

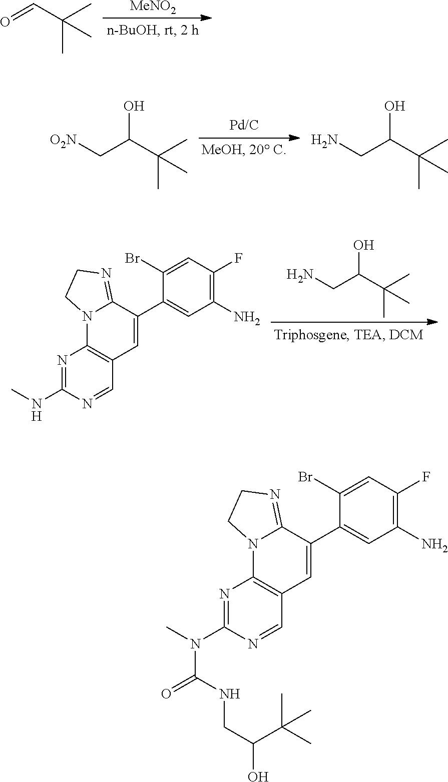

### Step 1: Synthesis of 3,3-dimethyl-1-nitrobutan-2-ol

To a solution of pivalaldehyde (0.5 g, 5.8 mmol) in toluene (10 mL) was added MeNO₂ (3 mL) dropwise at −60° C. After 30 min, n-BuOH (0.1 mL) was added. The mixture was stirred at rt for 2 h. TLC show a new point. The crude was diluted with EA (20 mL) and H₂O (20 mL). The organic layer was concentrated to give the crude 3,3-dimethyl-1-nitrobutan-2-ol (850 mg, 99% yield).

### Step 2: Synthesis of 1-amino-3,3-dimethylbutan-2-ol

To a solution of 3,3-dimethyl-1-nitrobutan-2-ol (300 mg, 4.4 mmol, 1.0 eq) in MeOH (10 mL) was added Pd/C (30 mg, 10%). The reaction mixture was stirred at 20° C. under H2 for 12 h. Filtration and concentration gave crude product 1-amino-3,3-dimethylbutan-2-ol (280 mg, 100%) as an oil.

### Step 3: Synthesis of 1-(6-(5-amino-2-bromo-4-fluorophenyl)-8,9-dihydroimidazo[1′,2′:1,6]pyrido[2,3-d]pyrimidin-2-yl)-3-(2-hydroxy-3,3-dimethylbutyl)-1-methylurea

A mixture of 6-(5-amino-2-bromo-4-fluorophenyl)-N-methyl-8,9-dihydroimidazo[1′,2′:1,6]pyrido[2,3-d]pyrimidin-2-amine (50 mg, 0.11 mmol, 1.0 eq) and TEA (33 mg, 0.33 mmol, 3.0 eq) in THF (15 mL) was added triphosgene (15 mg, 0.05 mmol, 0.4 eq). The mixture was stirred at 0° C. for 1 h. After 1 h, 1-amino-3,3-dimethylbutan-2-ol was added to the solution. The mixture was stirred at rt for 16 h. LCMS showed the reaction was OK. The resulting mixture was purified by prep-HPLC (0.1% HCl) to afford 1-(6-(5-amino-2-bromo-4-fluorophenyl)-8,9-dihydroimidazo[1′,2′:1,6]pyrido[2,3-d]pyrimidin-2-yl)-3-(2-hydroxy-3,3-dimethylbutyl)-1-methylurea (3.1 mg, 4.5% yield) as a yellow solid. ¹H NMR (400 MHz, CD₃OD): δ 9.16 (s, 1H), 8.27 (s, 1H), 7.50 (d, J=10.4 Hz, 1H), 7.09 (d, J=8.4 Hz, 1H), 5.04-4.96 (m, 2H), 4.24 (t, J=9.2 Hz, 2H), 3.82 (dd, J=13.6, 2.0 Hz, 1H), 3.65 (s, 3H), 3.40 (dd, J=10.0, 2.0 Hz, 1H), 3.13 (dd, J=13.6, 2.0 Hz, 1H), 0.98 (s, 9H). LCMS (M+H⁺) m/z: 532.0.

### Example 323: Preparation of 1-(4-bromo-2-fluoro-5-(2-(methylamino)-8,9-dihydroimidazo[1′,2′:1,6]pyrido[2,3-d]pyrimidin-6-yl)phenyl)-3-(3,3-dimethylbutyl)urea (Compound 323)

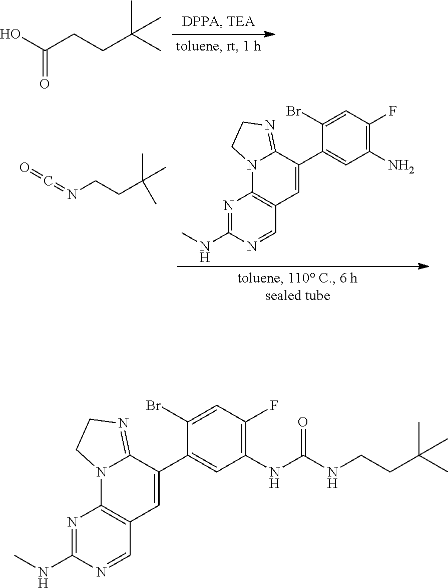

### Step 1: Synthesis of 1-(4-bromo-2-fluoro-5-(2-(methylamino)-8,9-dihydroimidazo[1′,2′:1,6]pyrido[2,3-d]pyrimidin-6-yl)phenyl)-3-(3,3-dimethylbutyl)urea

A mixture of 4,4-dimethylpentanoic acid (50 mg, 0.23 mmol) and TEA (70 mg, 0.69 mmol) in toluene (10 mL) was added DPPA (95 mg, 0.35 mmol), the mixture was stirred at 25° C. for 1 h. A mixture of 6-(5-amino-2-bromo-4-fluorophenyl)-N-methyl-8,9-dihydroimidazo[1′,2′:1,6]pyrido[2,3-d]pyrimidin-2-amine (50 mg, 0.15 mmol) in toluene (5 mL) was added to the solution. The mixture was stirred at 110° C. under N₂ for 12 h in sealed tube. LCMS show the reaction was ok. Concentration and the residue was purified by Prep-HPLC (0.1% HCl) to afford 1-(4-bromo-2-fluoro-5-(2-(methylamino)-8,9-dihydroimidazo[1′,2′:1,6]pyrido[2,3-d]pyrimidin-6-yl)phenyl)-3-(3,3-dimethylbutyl)urea (1.5 mg, 4% yield) as a yellow solid. ¹H NMR (400 MHz, CD₃OD): δ 8.87 (s, 1H), 8.24 (d, J=8.0 Hz, 1H), 8.09 (s, 1H), 7.62 (d, J=10.4 Hz, 1H), 4.76-4.73 (m, 2H), 4.19-4.14 (m, 2H), 3.27-3.15 (m, 2H), 3.10 (s, 3H), 1.48-1.44 (m, 2H), 1.03 (s, 9H). LCMS (M+H⁺) m/z: 516.1.

### Example 324: Preparation of 1-(4-chloro-2-fluoro-5-(2-(methylamino)-8,9-dihydroimidazo[1′,2′:1,6]pyrido[2,3-d]pyrimidin-6-yl)phenyl)-3-(3,3-dimethylbutyl)urea (Compound 324)

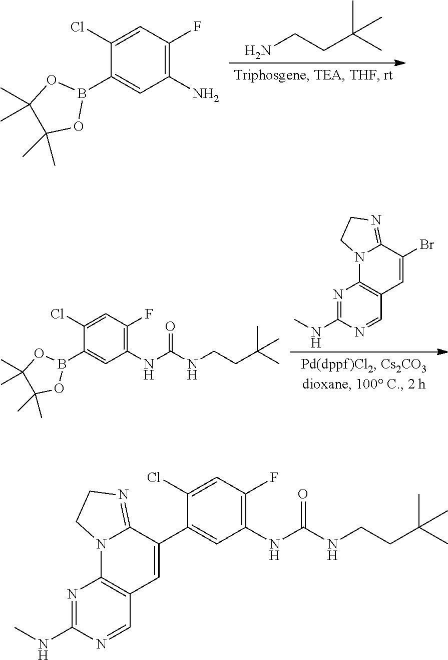

### Step 1: Synthesis of a mixture of 1-(4-chloro-2-fluoro-5-(4,4,5,5-tetramethyl-1,3,2-dioxaborolan-2-yl)phenyl)-3-(3,3-dimethylbutyl)urea

To a solution of 4-chloro-2-fluoro-5-(4,4,5,5-tetramethyl-1,3,2-dioxaborolan-2-yl)aniline (70 mg, 0.26 mmol) and TEA (79 mg, 0.78 mmol) in THF (6 mL) was added triphosgene (38 mg, 0.13 mmol). The solution stirred at r.t under N2 for 1 h. Then the reaction solution was added to a solution of 3,3-dimethylbutan-1-amine (26 mg, 0.26 mmol) in THF (2 mL). The mixture was stirred at r.t under N2 for 1 h. The mixture was filtered. The filtrate was diluted with EA (60 mL) and washed with water. The organic phase was concentrated to afford a mixture of 1-(4-chloro-2-fluoro-5-(4,4,5,5-tetramethyl-1,3,2-dioxaborolan-2-yl)phenyl)-3-(3,3-dimethylbutyl)urea (103 mg crude, 100% yield) as a yellow oil. LCMS (M+H⁺) m/z: 399.4.

### Step 2: Synthesis of 1-(4-chloro-2-fluoro-5-(2-(methylamino)-8,9-dihydroimidazo[1′,2′:1,6]pyrido[2,3-d]pyrimidin-6-yl)phenyl)-3-(3,3-dimethylbutyl)urea hydrochloride

A mixture of 1-(4-chloro-2-fluoro-5-(4,4,5,5-tetramethyl-1,3,2-dioxaborolan-2-yl)phenyl)-3-(3,3-dimethylbutyl)urea (103 mg crude, 0.26 mmol), 6-bromo-N-methyl-8,9-dihydroimidazo[1′,2′:1,6]pyrido[2,3-d]pyrimidin-2-amine (73 mg, 0.26 mmol), Cs2CO3 (253 mg, 0.78 mmol) and Pd(dppf)Cl2 (19 mg, 0.026 mmol) in dioxane (6 mL) and water (1.5 mL) was stirred at 100° C. under N2 for 5 h. The reaction mixture was cooled to r.t., diluted with EA (50 mL) and water (20 mL). The organic phase was concentrated and purified by prep-TLC (DCM/MeOH=10/1) and Prep-HPLC (0.1%/HCl/CH3CN/H2O) to afford 1-(4-chloro-2-fluoro-5-(2-(methylamino)-8,9-dihydroimidazo[1′,2′:1,6]pyrido[2,3-d]pyrimidin-6-yl)phenyl)-3-(3,3-dimethylbutyl)urea hydrochloride (14.45 mg, 10.9% yield) as a yellow solid. ¹H NMR (400 MHz, DMSO-d6) δ 9.90 (s, 1H), 8.89 (s, 1H), 8.70 (s, 1H), 8.63-8.45 (m, 1H), 8.36 (d, J=8.4 Hz, 1H), 8.18 (s, 1H), 7.66 (d, J=10.8 Hz, 1H), 6.80 (s, 1H), 4.71-4.60 (m, 2H), 4.06-4.00 (m, 2H), 3.12-3.08 (m, 2H), 2.96 (d, J=4.8 Hz, 3H), 1.37-1.33 (m, 2H), 0.90 (s, 9H). LCMS (M+H)⁺ m/z: 472.1.

### Example 325: Preparation of 1-(6-(5-amino-2-chloro-4-fluorophenyl)-8,9-dihydroimidazo[1′,2′:1,6]pyrido[2,3-d]pyrimidin-2-yl)-3-(3,3-dimethylbutyl)-1-methylurea (Compound 325)

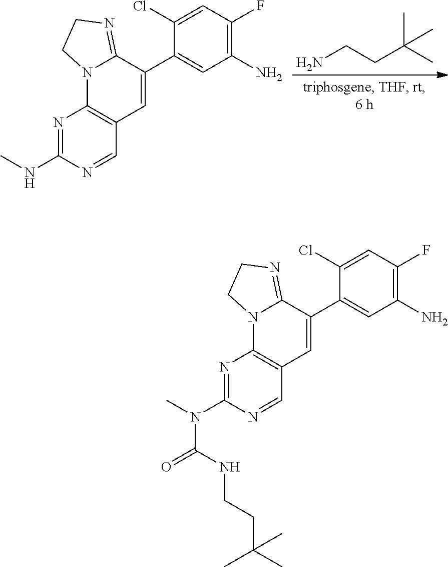

### Step 1: Synthesis of 1-(6-(5-amino-2-chloro-4-fluorophenyl)-8,9-dihydroimidazo[1′,2′:1,6]pyrido[2,3-d]pyrimidin-2-yl)-3-(3,3-dimethylbutyl)-1-methylurea hydrogen chloride

To a mixture of 6-(5-amino-2-chloro-4-fluorophenyl)-N-methyl-8,9-dihydroimidazo[1′,2′:1,6]pyrido[2,3-d]pyrimidin-2-amine (53 mg, 0.14 mmol) in THF (8 mL) was added triphosgene (17 mg, 0.056 mmol). The mixture was stirred at rt under N2 for 1 hr. Then a solution of 3,3-dimethylbutan-1-amine (10 mg, 0.098 mmol) and TEA (113 mg, 1.12 mmol) was added. The mixture was stirred at rt under N2 for 16 hrs and concentrated. The residue was purified by prep-HPLC (0.1%/HCl/CH3CN/H2O) to afford 1-(6-(5-amino-2-chloro-4-fluorophenyl)-8,9-dihydroimidazo[1′,2′:1,6]pyrido[2,3-d]pyrimidin-2-yl)-3-(3,3-dimethylbutyl)-1-methylurea hydrogen chloride (2.9 mg, 4% yield) as a yellow solid. ¹H NMR (400 MHz, DMSO-d₆): δ 10.55 (s, 1H), 9.57 (t, J=5.2 Hz, 1H), 9.21 (s, 1H), 8.38 (s, 1H), 7.42 (d, J=11.2 Hz, 1H), 6.85 (d, J=9.2 Hz, 1H), 4.74-4.71 (m, 2H), 4.17-4.12 (m, 2H), 3.49 (s, 3H), 3.23-3.18 (m, 2H), 1.52-1.48 (m, 2H), 0.95 (s, 9H). LCMS (M+H⁺) m/z: 472.1.

### Example 326: Preparation of 1-(4-bromo-2-fluoro-5-(2-(methylamino)-8,9-dihydroimidazo[1′,2′:1,6]pyrido[2,3-d]pyrimidin-6-yl)phenyl)-3-phenylurea (Compound 326)

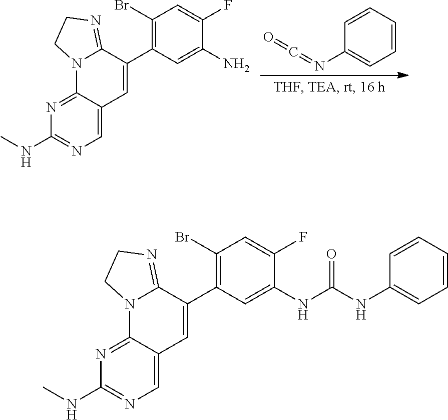 [SMILES: CNc1ncc2c(n1)N1CCN=C1C(c1cc(N)c(F)cc1Br)=C2 ; O=C=Nc1ccccc1 ; CNc1ncc2c(n1)N1CCN=C1C(c1cc(NC(=O)Nc3ccccc3)c(F)cc1Br)=C2]

### Step 1: Synthesis of 1-(4-bromo-2-fluoro-5-(2-(methylamino)-8,9-dihydroimidazo[1′,2′:1,6]pyrido[2,3-d]pyrimidin-6-yl)phenyl)-3-phenylurea

To a solution of 6-(5-amino-2-bromo-4-fluorophenyl)-N-methyl-8,9-dihydroimidazo[1′,2′:1,6]pyrido[2,3-d]pyrimidin-2-amine (50 mg, 0.129 mmol) and isocyanatobenzene (16 mg, 0.135 mmol) in THF (20 mL) was added TEA (39 mg, 0.441 mmol), the mixture reaction was stirred at rt for 16 h. The solvent was removed and then purified by prep-HPLC (0.1% TFA) to afford 1-(4-bromo-2-fluoro-5-(2-(methylamino)-8,9-dihydroimidazo[1′,2′:1,6]pyrido[2,3-d]pyrimidin-6-yl)phenyl)-3-phenylurea as (7.5 mg, 11.4% yield) a yellow solid. ¹H NMR (400 MHz, CD₃OD): δ 8.80 (s, 1H), 8.34 (d, J=8.0 Hz, 1H), 8.09 (s, 1H), 7.65 (d, J=10.4 Hz, 1H), 7.42 (dd, J=8.4, 1.2 Hz, 2H), 7.28 (t, J=8.0 Hz, 2H), 7.05-7.01 (m, 1H), 4.80-4.77 (m, 2H), 4.16 (t, J=10.0 Hz, 2H), 3.08 (s, 3H). LCMS (M+H⁺) m/z: 508.0.

### Example 327: Preparation of 1-(4-bromo-2-fluoro-5-(2-(methylamino)-8,9-dihydroimidazo[1′,2′:1,6]pyrido[2,3-d]pyrimidin-6-yl)phenyl)-3-(2-chlorophenyl)urea (Compound 327)

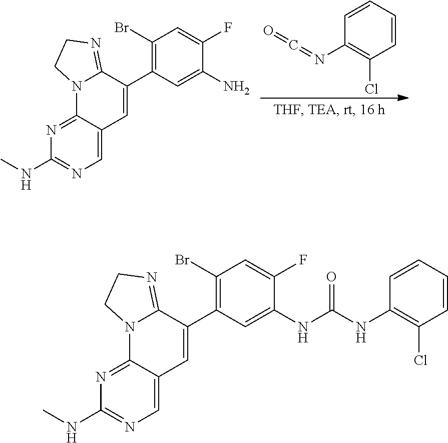

### Step 1: Synthesis of 1-(4-bromo-2-fluoro-5-(2-(methylamino)-8,9-dihydroimidazo[1′,2′:1,6]pyrido[2,3-d]pyrimidin-6-yl)phenyl)-3-(2-chlorophenyl)urea

To a solution of 6-(5-amino-2-bromo-4-fluorophenyl)-N-methyl-8,9-dihydroimidazo[1′,2′:1,6]pyrido[2,3-d]pyrimidin-2-amine (50 mg, 0.129 mmol) and 1-chloro-2-isocyanatobenzene (20 mg, 0.135 mmol) in THF (20 mL) was added TEA (39 mg, 0.387 mmol). The mixture reaction was stirred at rt for 16 h. The solvent was removed and the residue was purified by prep-HPLC (0.1% HCl) to afford 1-(4-bromo-2-fluoro-5-(2-(methylamino)-8,9-dihydroimidazo[1′,2′:1,6]pyrido[2,3-d]pyrimidin-6-yl)phenyl)-3-(2-chlorophenyl)urea (10.1 mg, 14.5% yield) as a yellow solid. ¹H NMR (400 MHz, CD₃OD): δ 8.83 (s, 1H), 8.40 (d, J=8.0 Hz, 1H), 8.12 (s, 1H), 8.08 (dd, J=8.8, 1.6 Hz, 1H), 7.69 (d, J=10.8 Hz, 1H), 7.43 (dd, J=8.0, 1.6 Hz, 1H), 7.30-7.26 (m, 1H), 7.10-7.08 (m, 1H), 4.86-4.82 (m, 2H), 4.20-4.16 (m, 2H), 3.11 (s, 3H). LCMS (M+H⁺) m/z: 542.1.

### Example 328: Preparation of 1-(4-bromo-2-fluoro-5-(2-(oxetan-3-ylamino)-8,9-dihydroimidazo[1′,2′:1,6]pyrido[2,3-d]pyrimidin-6-yl)phenyl)-3-phenylurea (Compound 328)

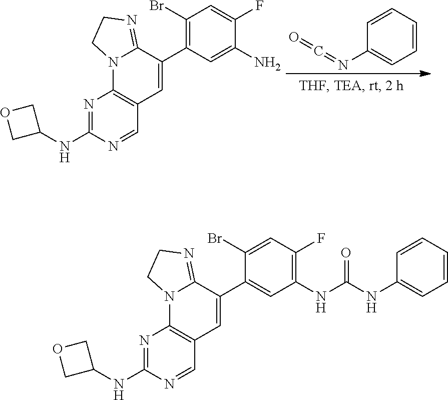 [SMILES: Nc1cc(C2=Cc3cnc(NC4COC4)nc3N3CCN=C23)c(Br)cc1F ; O=C=Nc1ccccc1 ; O=C(Nc1ccccc1)Nc1cc(C2=Cc3cnc(NC4COC4)nc3N3CCN=C23)c(Br)cc1F]

### Step 1: Synthesis of 1-(4-bromo-2-fluoro-5-(2-(oxetan-3-ylamino)-8, 9-dihydroimidazo[1′, 2′:1, 6]pyrido[2, 3-d]pyrimidin-6-yl)phenyl)-3-phenylurea

To a mixture of 6-(5-amino-2-bromo-4-fluorophenyl)-N-(oxetan-3-yl)-8,9-dihydroimidazo[1′,2′:1,6]pyrido[2,3-d]pyrimidin-2-amine (50 mg, 0.12 mmol, 1 eq) in THF (10 mL) was added TEA (5 drops, 0.6 mmol, 5 eq) and isocyanatobenzene (1.5 drops, 0.24 mmol, 2 eq). The mixture was stirred at rt under N2 for 2 h. The resulting mixture was purified by prep-HPLC (0.1% NH3·H2O) to afford 1-(4-bromo-2-fluoro-5-(2-(oxetan-3-ylamino)-8, 9-dihydroimidazo[1′, 2′:1, 6]pyrido[2, 3-d]pyrimidin-6-yl)phenyl)-3-phenylurea (6.4 mg, 9.7% yield) as a yellow solid. ¹H NMR (400 MHz, DMSO-d₆): δ 9.12 (s, 1H), 8.71 (s, 1H), 8.28-8.20 (m, 3H), 7.65 (d, J=10.8 Hz, 1H), 7.43 (d, J=7.6 Hz, 2H), 7.28 (t, J=7.6 Hz, 2H), 7.17 (s, 1H), 6.99 (t, J=7.6 Hz, 1H), 4.96-4.92 (m, 1H), 4.76 (t, J=6.4 Hz, 2H), 4.54 (t, J=6.4 Hz, 2H), 4.03-3.88 (m, 4H). LCMS (M+H⁺) m/z: 550.0.

### Example 329: Preparation of 1-(4-bromo-2-fluoro-5-(2-(oxetan-3-ylamino)-8,9-dihydroimidazo[1′,2′:1,6]pyrido[2,3-d]pyrimidin-6-yl)phenyl)-3-(2-chlorophenyl)urea (Compound 329)

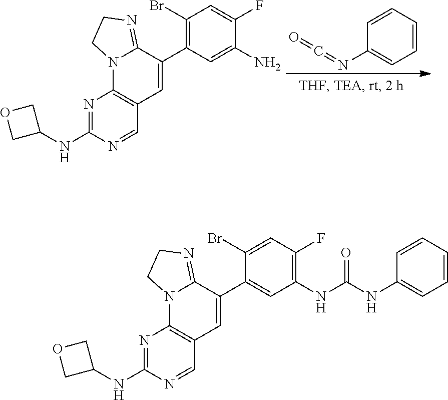 [SMILES: Nc1cc(C2=Cc3cnc(NC4COC4)nc3N3CCN=C23)c(Br)cc1F ; O=C=Nc1ccccc1 ; O=C(Nc1ccccc1)Nc1cc(C2=Cc3cnc(NC4COC4)nc3N3CCN=C23)c(Br)cc1F]

### Step 1; Synthesis of 1-(4-bromo-2-fluoro-5-(2-(oxetan-3-ylamino)-8,9-dihydroimidazo[1′,2′:1,6]pyrido[2,3-d]pyrimidin-6-yl)phenyl)-3-(2-chlorophenyl)urea

To a solution of 6-(5-amino-2-bromo-4-fluorophenyl)-N-(oxetan-3-yl)-8,9-dihydroimidazo[1′,2′:1,6]pyrido[2,3-d]pyrimidin-2-amine (50 mg, 0.116 mmol) and isocyanatobenzene (19 mg, 0.122 mmol) in THF (20 mL) was added TEA (35 mg, 0.348 mmol), the mixture reaction was stirred at rt for 4 h. The solvent was removed and then purified by prep-HPLC (0.1% NH₃H₂O) to afford 1-(4-bromo-2-fluoro-5-(2-(oxetan-3-ylamino)-8,9-dihydroimidazo[1′,2′:1,6]pyrido[2,3-d]pyrimidin-6-yl)phenyl)-3-(2-chlorophenyl)urea (19.9 mg, 29.4% yield) as a yellow solid. ¹H NMR (400 MHz, DMSO-d₆): δ 9.50 (s, 1H), 8.84 (s, 1H), 8.29-8.22 (m, 3H), 8.08 (dd, J=8.4, 1.6 Hz, 1H), 7.69 (d, J=10.8 Hz, 1H), 7.46 (dd, J=8.0, 1.2 Hz, 1H), 7.31-7.26 (m, 1H), 7.18-7.15 (m, 1H), 7.05 (td, J=8.0, 1.6 Hz, 1H), 4.97-4.94 (m, 1H), 4.79-4.76 (m, 2H), 4.56-4.53 (m, 2H), 4.04-3.97 (m, 2H), 3.93-3.88 (m, 2H). LCMS (M+H⁺) m/z: 584.0.

### Example 330: Preparation of 1-(2-fluoro-4-methyl-5-(2-(methylamino)-8,9-dihydroimidazo[1′,2′:1,6]pyrido[2,3-d]pyrimidin-6-yl)phenyl)-3-(3-(trifluoromethyl)phenyl)urea (Compound 330)

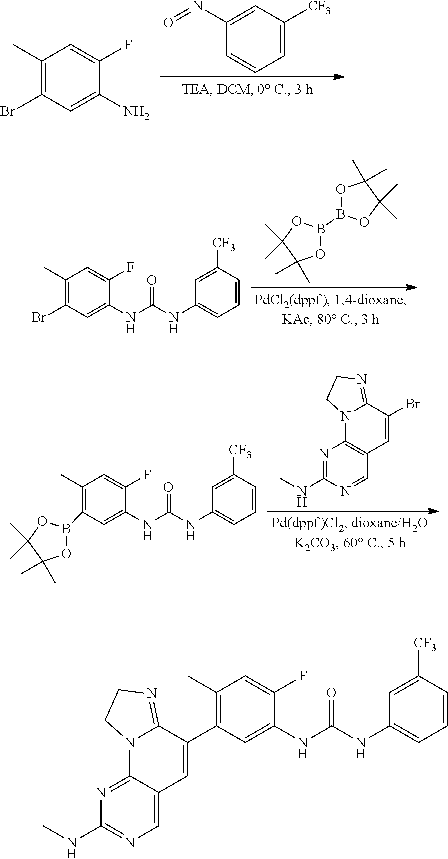

### Step 1: Synthesis of 1-(5-bromo-2-fluoro-4-methylphenyl)-3-(3-(trifluoromethyl)phenyl)urea

To a solution of 5-bromo-2-fluoro-4-methylaniline (100 mg, 0.49 mmol) in DCM (9 mL). were added 1-isocyanato-3-(trifluoromethyl)benzene (100 mg, 0.539 mmol) and TEA (123 mg, 1.225 mmol) at 0° C. The mixture was stirred at 0° C. for 2 h. Water was added, the organic layer was separated and concentrated, the residue was purified by flash chromatography on silica gel to give 1-(5-bromo-2-fluoro-4-methylphenyl)-3-(3-(trifluoromethyl)phenyl)urea (140 mg, 66% yield). LCMS (M+H⁺) m/z: 391.0

### Step 2: Synthesis of 1-(2-fluoro-4-methyl-5-(4,4,5,5-tetramethyl-1,3,2-dioxaborolan-2-yl)phenyl)-3-(3-(trifluoromethyl)phenyl)urea

1-(5-Bromo-2-fluoro-4-methylphenyl)-3-(3-(trifluoromethyl)phenyl)urea (147 mg, 0.5 mmol, 1.0 equiv), Pin₂B2 (190 mg, 0.75 mmol, 1.5 equiv), KOAc (150 mg, 1.5 mmol, 3.0 equiv), and PdCl₂ (dppf) (73 mg, 0.1 mmol, 20 mol %) in 1, 4-dioxane (4 mL) was refluxed for 16 h under N₂. The crude 1-(2-fluoro-4-methyl-5-(4,4,5,5-tetramethyl-1,3,2-dioxaborolan-2-yl)phenyl)-3-(3-(trifluoromethyl)phenyl)urea was used for next stepwithout further purification. LCMS (M+H⁺) m/z: 439.0

### Step 4: Synthesis of 1-(2-fluoro-4-methyl-5-(2-(methylamino)-8,9-dihydroimidazo[1′,2′:1,6]pyrido[2,3-d]pyrimidin-6-yl)phenyl)-3-(3-(trifluoromethyl)phenyl)urea

The 1-(2-fluoro-4-methyl-5-(4,4,5,5-tetramethyl-1,3,2-dioxaborolan-2-yl)phenyl)-3-(3-(trifluoromethyl)phenyl)urea (44 mg, 0.1 mmol, 1 equiv), 6-bromo-N-methyl-8,9-dihydroimidazo[1′,2′:1,6]pyrido[2,3-d]pyrimidin-2-amine (56 mg, 0.2 mmol, 1.0 equiv), K₂CO₃ (69 mg, 0.5 mmol, 3.0 equiv), and PdCl₂ (dppf) (29 mg, 0.04 mmol, 40 mol %) in 1, 4-dioxane (4.0 mL) and H₂O (1.0 mL) was stirred at 60° C. for 5 h under N₂. The reaction mixture was concentrated and purified by Prep-HPLC to give 1-(2-fluoro-4-methyl-5-(2-(methylamino)-8,9-dihydroimidazo[1′,2′:1,6]pyrido[2,3-d]pyrimidin-6-yl)phenyl)-3-(3-(trifluoromethyl)phenyl)urea (6.3 mg, 6% yield) as a solid. ¹H NMR (400 MHz, DMSO-d₆): δ 9.37 (s, 1H), 8.60 (s, 1H), 8.25 (br, 1H), 8.04 (s, 1H), 7.94 (d, J=8.4 Hz, 1H), 7.53-7.47 (m, 3H), 7.32 (d, J=7.2 Hz, 1H), 7.16-7.13 (m, 2H), 4.08-3.88 (m, 4H), 2.84 (s, 3H), 2.16 (s, 3H). LCMS (M+H⁺) m/z: 512.0.

### Example 331: Preparation of 1-(2-chlorophenyl)-3-(2-fluoro-4-methyl-5-(2-(methylamino)-8,9-dihydroimidazo[1′,2′:1,6]pyrido[2,3-d]pyrimidin-6-yl)phenyl)urea (Compound 331)

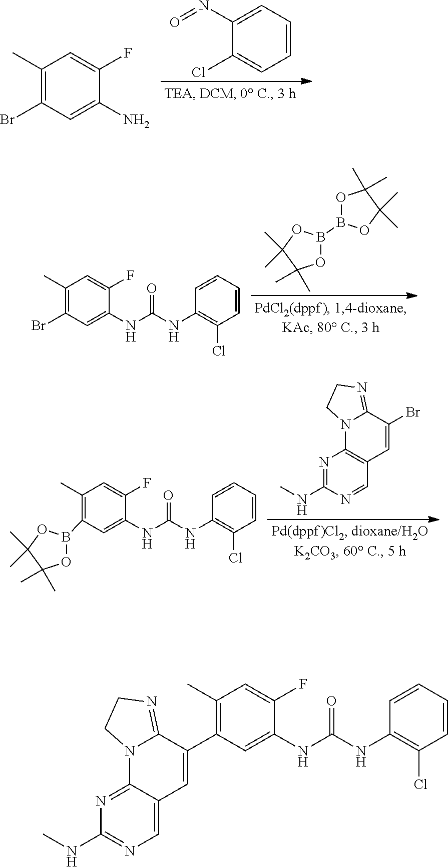

### Step 1: Synthesis of 1-(5-bromo-2-fluoro-4-methylphenyl)-3-(2-chlorophenyl)urea

To a solution of 5-bromo-2-fluoro-4-methylaniline (150 mg, 0.735 mmol) in DCM (3 mL) was added 1-chloro-2-isocyanatobenzene (168 mg, 1.102 mmol) and TEA (185 mg, 1.837 mmol) at 0° C., The mixture was stirred at 0° C. for 2 h. Water was added, the organic layer was separated and concentrated, the residue was purified by flash chromatography on silica gel to give 1-(5-bromo-2-fluoro-4-methylphenyl)-3-(2-chlorophenyl)urea (130 mg, 50% yield). LCMS (M+H⁺) m/z: 357.0

### Step 2: Synthesis of 1-(2-chlorophenyl)-3-(2-fluoro-4-methyl-5-(4,4,5,5-tetramethyl-1,3,2-dioxaborolan-2-yl)phenyl)urea

A mixture of 1-(5-bromo-2-fluoro-4-methylphenyl)-3-(2-chlorophenyl)urea (70 mg, 0.197 mmol) in dioxane (3 mL) was added Pin2B2 (75 mg, 0.295 mmol), Pd(dppf)Cl₂ (21 mg, 0.0295 mmol) and KOAc (58 mg, 0.591 mmol) was stirred at 85° C. for 4 h. The crude 1-(2-chlorophenyl)-3-(2-fluoro-4-methyl-5-(4,4,5,5-tetramethyl-1,3,2-dioxaborolan-2-yl)phenyl)urea was used to next step without further purification.

### Step 3: Synthesis of 1-(2-chlorophenyl)-3-(2-fluoro-4-methyl-5-(2-(methylamino)-8,9-dihydroimidazo[1′,2′:1,6]pyrido[2,3-d]pyrimidin-6-yl)phenyl)urea

To a solution of 6-bromo-N-methyl-8,9-dihydroimidazo[1′,2′:1,6]pyrido[2,3-d]pyrimidin-2-amine (200 mg, 0.713 mmol) in dioxane (3 mL) was added crude 1-(2-chlorophenyl)-3-(2-fluoro-4-methyl-5-(4,4,5,5-tetramethyl-1,3,2-dioxaborolan-2-yl)phenyl)urea (346 mg, 0.856 mmol), Pd(dppf)Cl₂ (78 mg, 0.106 mmol) and K₂CO₃ (295 mg, 2.139 mmol). The mixture was stirred at 85° C. for 4 h. The mixture was concentrated to give the crude product, which was purified by prep-HPLC to give 1-(2-chlorophenyl)-3-(2-fluoro-4-methyl-5-(2-(methylamino)-8,9-dihydroimidazo[1′,2′:1,6]pyrido[2,3-d]pyrimidin-6-yl)phenyl)urea (11.1 mg, yield 3%). ¹H NMR (400 MHz, DMSO-d6): δ 9.31 (s, 1H), 8.75 (s, 1H), 8.25 (br, 1H), 8.11 (dd, J=8.4, 1.6 Hz, 1H), 7.98 (d, J=8.4 Hz, 1H), 7.45 (dd, J=8.0, 1.2 Hz, 2H), 7.28-7.26 (m, 1H), 7.15 (d, J=12.0 Hz, 2H), 7.03-7.02 (m, 1H), 4.10-3.90 (m, 4H), 2.84 (s, 3H) 2.16 (s, 3H). LCMS (M+H⁺) m/z: 478.0.

### Example 332: Preparation of 1-(2-chlorophenyl)-3-(5-(2-(ethylamino)-8,9-dihydroimidazo[1′,2′:1,6]pyrido[2,3-d]pyrimidin-6-yl)-2-fluoro-4-methylphenyl)urea (Compound 332)

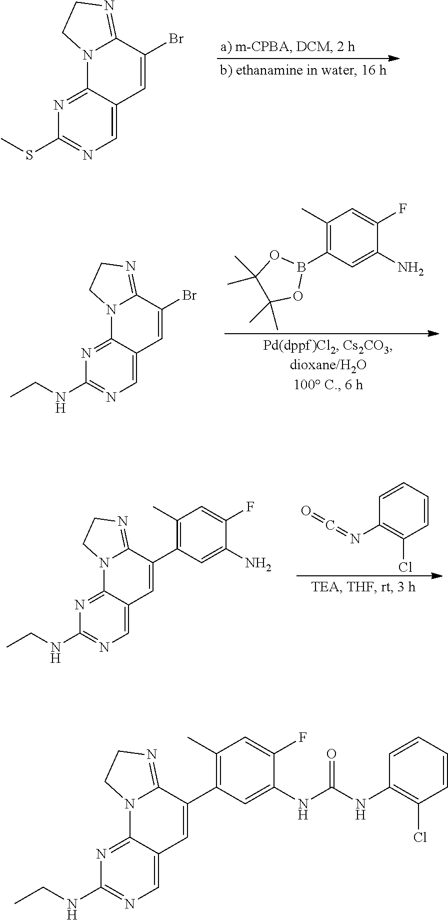 [SMILES: CSc1ncc2c(n1)N1CCN=C1C(Br)=C2 ; CCNc1ncc2c(n1)N1CCN=C1C(Br)=C2 ; Cc1cc(F)c(N)cc1B1OC(C)(C)C(C)(C)O1 ; CCNc1ncc2c(n1)N1CCN=C1C(c1cc(N)c(F)cc1C)=C2 ; O=C=Nc1ccccc1Cl ; CCNc1ncc2c(n1)N1CCN=C1C(c1cc(NC(=O)Nc3ccccc3Cl)c(F)cc1C)=C2]

### Step 1: Synthesis of 6-bromo-N-ethyl-8,9-dihydroimidazo[1′,2′:1,6]pyrido[2,3-d]pyrimidin-2-amine

To a solution of 6-bromo-2-(methylthio)-8,9-dihydroimidazo[1′,2′:1,6]pyrido[2,3-d]pyrimidine (420 mg, 1.41 mmol) in DCM (10 mL) was added m-CPBA (1039 mg, 4.23 mmol). The solution stirred at r.t under N2 for 1 h. Then ethanamine (634 mg, 8.46 mmol, 60% in water) was added. The solution was stirred at r.t under N2 for 1 h. The solution was diluted with sat. NaHCO₃ (30 mL) and extracted with DCM/MeOH=10/1 (30 mL×3). The combined organic phase was concentrated and purified by flash chromatography (DCM/MeOH=10/1) to afford 6-bromo-N-ethyl-8,9-dihydroimidazo[1′,2′:1,6]pyrido[2,3-d]pyrimidin-2-amine (320 mg, 77% yield) as a yellow solid. LCMS (M+H⁺) m/z: 296.0.

### Step 2: Synthesis of 6-(5-amino-4-fluoro-2-methylphenyl)-N-ethyl-8,9-dihydroimidazo[1′,2′:1,6]pyrido[2,3-d]pyrimidin-2-amine

A mixture of 6-bromo-N-ethyl-8,9-dihydroimidazo[1′,2′:1,6]pyrido[2,3-d]pyrimidin-2-amine (150 mg, 0.51 mmol), 2-fluoro-4-methyl-5-(4,4,5,5-tetramethyl-1,3,2-dioxaborolan-2-yl)aniline (128 mg, 0.51 mmol), Cs2CO3 (497 mg, 1.53 mmol) and Pd(dppf)Cl2 (75 mg, 0.102 mmol) in dioxane (6 mL) and water (1.5 mL) was stirred at 100° C. under N2 for 6 h. The reaction mixture was cooled to r.t. and purified by flash chromatography (0.1%/FA/CH3CN/H2O) to afford 6-(5-amino-4-fluoro-2-methylphenyl)-N-ethyl-8,9-dihydroimidazo[1′,2′:1,6]pyrido[2,3-d]pyrimidin-2-amine (153 mg, 89% yield) as a yellow solid. LCMS (M+H⁺) m/z: 339.3.

### Step 3: Synthesis of 1-(2-chlorophenyl)-3-(5-(2-(ethylamino)-8,9-dihydroimidazo[1′,2′:1,6]pyrido[2,3-d]pyrimidin-6-yl)-2-fluoro-4-methylphenyl)urea hydrochloride

To a mixture of 6-(5-amino-4-fluoro-2-methylphenyl)-N-ethyl-8,9-dihydroimidazo[1′,2′:1,6]pyrido[2,3-d]pyrimidin-2-amine (80 mg, 0.24 mmol) and TEA (73 mg, 0.72 mmol) in THF (6 mL) was added 1-chloro-2-isocyanatobenzene (37 mg, 0.24 mmol). The reaction mixture was stirred at R.T under N2 for 3 hour. The mixture was filtered and the filter cake was purified by prep-HPLC (0.1%/HCl/CH3CN/H2O) to afford 1-(2-chlorophenyl)-3-(5-(2-(ethylamino)-8,9-dihydroimidazo[1′,2′:1,6]pyrido[2,3-d]pyrimidin-6-yl)-2-fluoro-4-methylphenyl)urea hydrochloride (19.8 mg, 15.8% yield) as a yellow solid. ¹H NMR (400 MHz, DMSO-d₆): δ 9.68 (s, 1H), 9.55 (s, 1H), 8.94-8.87 (m, 2H), 8.59-7.90 (m, 1H), 8.13-8.07 (m, 3H), 7.46 (d, J=7.2 Hz, 1H), 7.34-7.26 (m, 2H), 7.04 (t, J=7.2 Hz, 1H), 4.64-4.59 (m, 2H), 4.01 (t, J=9.6 Hz, 2H), 3.48-3.42 (m, 2H), 2.16 (s, 3H), 1.23-1.15 (m, 3H). LCMS (M+H⁺) m/z: 492.0.

### Example 333: Preparation of 1-(2-fluoro-4-methyl-5-(2-(methylamino)-8,9-dihydroimidazo[1′,2′:1,6]pyrido[2,3-d]pyrimidin-6-yl)phenyl)-3-(2-fluoro-5-(trifluoromethyl)phenyl)urea (Compound 333)

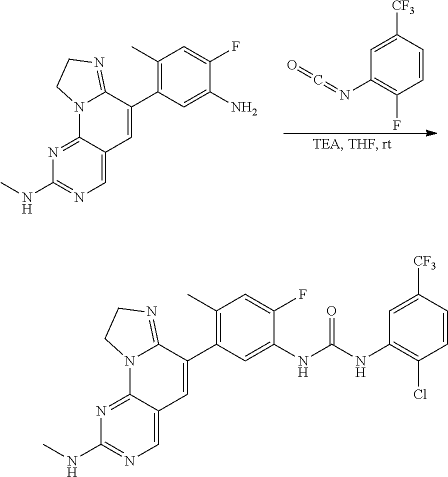 [SMILES: CNc1ncc2c(n1)N1CCN=C1C(c1cc(N)c(F)cc1C)=C2 ; O=C=Nc1cc(C(F)(F)F)ccc1F ; CNc1ncc2c(n1)N1CCN=C1C(c1cc(NC(=O)Nc3cc(C(F)(F)F)ccc3Cl)c(F)cc1C)=C2]

### Step 1: Synthesis of 1-(2-fluoro-4-methyl-5-(2-(methylamino)-8,9-dihydroimidazo[1′,2′:1,6]pyrido[2,3-d]pyrimidin-6-yl)phenyl)-3-(2-fluoro-5-(trifluoromethyl)phenyl)urea

To a mixture of 6-(5-amino-4-fluoro-2-methylphenyl)-N-methyl-8,9-dihydroimidazo[1′,2′:1,6]pyrido[2,3-d]pyrimidin-2-amine (40 mg, 0.12 mmol) and TEA (48 mg, 0.48 mmol) in THF (5 mL) was added 1-fluoro-2-isocyanato-4-(trifluoromethyl)benzene (25 mg, 0.12 mmol). The reaction mixture was stirred at R.T under N2 for 1 hour. The mixture was concentrated and purified by prep-HPLC (0.1%/HCl/CH3CN/H2O) to afford 1-(2-fluoro-4-methyl-5-(2-(methylamino)-8,9-dihydroimidazo[1′,2′:1,6]pyrido[2,3-d]pyrimidin-6-yl)phenyl)-3-(2-fluoro-5-(trifluoromethyl)phenyl)urea (13 mg, 18% yield) as a yellow solid. ¹H NMR (400 MHz, DMSO-d₆): δ 9.71 (s, 1H), 9.62 (s, 1H), 9.51 (s, 1H), 8.87 (s, 1H), 8.60 (d, J=1.6 Hz, 1H), 8.59-8.51 (m, 1H), 8.14 (d, J=8.4 Hz, 1H), 8.11 (s, 1H), 7.53-7.48 (m, 1H), 7.40-7.38 (m, 1H), 7.34 (d, J=12.0 Hz, 1H), 4.66-4.54 (m, 2H), 4.02 (t, J=9.6 Hz, 2H), 2.96 (d, J=4.4 Hz, 3H), 2.16 (s, 3H). LCMS (M+H⁺) m/z: 530.3.

### Example 334: Preparation of 1-(2-chloro-4-methyl-5-(2-(methylamino)-8,9-dihydroimidazo[1′,2′:1,6]pyrido[2,3-d]pyrimidin-6-yl)phenyl)-3-(2-fluorophenyl)urea (Compound 334)

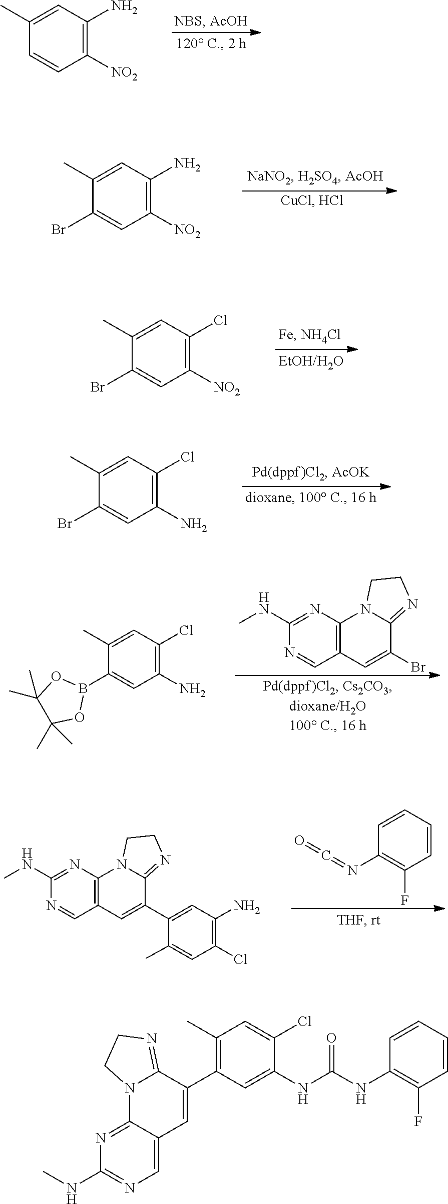

### Step 1: Synthesis of 4-bromo-5-methyl-2-nitroaniline

To a solution of 5-methyl-2-nitroaniline (2.5 g, 16.4 mmol) in CH3COOH (40 mL) was added NBS (2.9 g, 16.1 mmol) dropwise at 20° C. The reaction mixture was stirred at 120° C. for 2 h. H2O (100 mL) was added and filtrated, the solid was dried under vacuum to afford 4-bromo-5-methyl-2-nitroaniline (3.3 g, 87% yield) as a yellow solid.

### Step 2: Synthesis of 1-bromo-4-chloro-2-methyl-5-nitrobenzene

To a solution of 4-bromo-5-methyl-2-nitroaniline (1 g, 4.4 mmol) in CH₃COOH (10 mL) was added to the solution of NaNO₂ in H₂SO₄ (5 mL) slowly at 0° C. The reaction mixture was stirred at 0° C. for 30 min, CuCl (1 g, 10 mmol) in HCl (5 mL) was added to the reaction solution, the reaction mixture was stirred at 60° C. for 2 h, LCMS show the reaction was ok. H₂O (50 mL) was added, the reaction mixture was extracted with EA. The combined extracts were washed with brine (20 mL), concentrated to give the crude product 1-bromo-4-chloro-2-methyl-5-nitrobenzene (0.9 g, 85% yield) as a yellow solid.

### Step 3: Synthesis of 5-bromo-2-chloro-4-methylaniline

A mixture of 1-bromo-4-chloro-2-methyl-5-nitrobenzene (0.9 g, 3.6 mmol) and Fe (203 mg, 3.6 mmol), NH₄Cl (187 mg, 3.6 mmol) in EtOH/H₂O (10 mL/10 mL) was stirred at 90° C. for 2 h. Concentration in vacuum and purification on silica gel column (PE/EA 5:1) afforded 5-bromo-2-chloro-4-methylaniline (800 mg, 87% yield) as a brown solid.

### Step 4: Synthesis of 2-chloro-4-methyl-5-(4,4,5,5-tetramethyl-1,3,2-dioxaborolan-2-yl)aniline

To a solution of 5-bromo-2-chloro-4-methylaniline (300 mg, 1.4 mmol), bis(pinacolato)diboron (500 mg, 2.1 mmol) and CH₃COOK (400 mg, 3 mmol) in dry dioxane (20 mL), was added Pd(dppf)Cl₂ (55 mg, 0.07 mmol). The mixture was stirred under N₂ at 100° C. for 16 h. Concentration in vacuum gave the residue, which was purified on silica gel column (PE:EA 10:1 to 1:1) to afford 2-chloro-4-methyl-5-(4,4,5,5-tetramethyl-1,3,2-dioxaborolan-2-yl)aniline (230 mg, 60% yield) as a white solid.

### Step 5: Synthesis of 6-(5-amino-4-chloro-2-methylphenyl)-N-methyl-8,9-dihydroimidazo[1′,2′:1,6]pyrido[2,3-d]pyrimidin-2-amine

To a mixture of 2-chloro-4-methyl-5-(4,4,5,5-tetramethyl-1,3,2-dioxaborolan-2-yl)aniline (100 mg, 0.37 mmol) and 6-bromo-N-methyl-8,9-dihydroimidazo[1′,2′:1,6]pyrido[2,3-d]pyrimidin-2-amine (103 mg, 0.37 mmol) in dioxane/H₂O (10 mL/1 mL) was added Cs2CO3 (361 mg, 1.1 mmol) and Pd(dppf)Cl₂ (15 mg, 0.02 mmol). The mixture was stirred at 100° C. overnight. Concentration in vacuum and purification by silica gel column (MeOH:DCM 50:1 to 10:1) afforded 6-(5-amino-4-chloro-2-methylphenyl)-N-methyl-8,9-dihydroimidazo[1′,2′:1,6]pyrido[2,3-d]pyrimidin-2-amine (100 mg, 53% yield) as a black solid.

### Step 6: Synthesis of 1-(2-chloro-4-methyl-5-(2-(methylamino)-8,9-dihydroimidazo[1′,2′:1,6]pyrido[2,3-d]pyrimidin-6-yl)phenyl)-3-(2-fluorophenyl)urea

To a mixture of 6-(5-amino-4-chloro-2-methylphenyl)-N-methyl-8,9-dihydroimidazo[1′,2′:1,6]pyrido[2,3-d]pyrimidin-2-amine (50 mg, 0.15 mmol) and 1-fluoro-2-isocyanatobenzene (30 mg, 0.22 mmol) in THF (15 mL) was added TEA (0.2 mL). The mixture was stirred at 0° C. under N2 overnight. The resulting mixture was concentrated and purified by prep-HPLC (0.1% HCl) to afford 1-(2-chloro-4-methyl-5-(2-(methylamino)-8,9-dihydroimidazo[1′,2′:1,6]pyrido[2,3-d]pyrimidin-6-yl)phenyl)-3-(2-fluorophenyl)urea (23.6 mg, 45% yield) as a yellow solid. ¹H NMR (400 MHz, CD₃OD): δ 8.86 (s, 1H), 8.07-8.03 (m, 3H), 7.49 (s, 1H), 7.16-7.01 (m, 3H), 4.83-4.69 (m, 2H), 4.14 (t, J=10.0 Hz, 2H), 3.06 (s, 3H), 2.23 (s, 3H). LCMS (M+H⁺) m/z: 478.1.

### Example 335: Preparation of 1-(2-chloro-5-(trifluoromethyl)phenyl)-3-(2-fluoro-4-methyl-5-(2-(methylamino)-8,9-dihydroimidazo[1′,2′:1,6]pyrido[2,3-d]pyrimidin-6-yl)phenyl)urea (Compound 335)

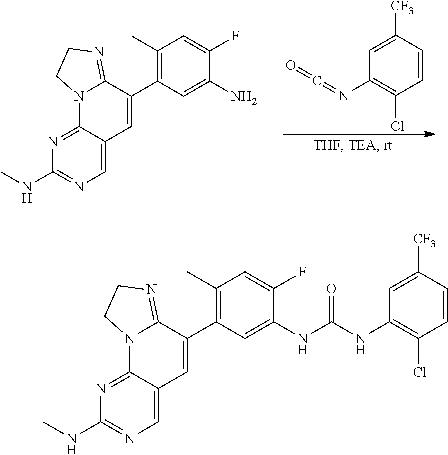 [SMILES: CNc1ncc2c(n1)N1CCN=C1C(c1cc(N)c(F)cc1C)=C2 ; O=C=Nc1cc(C(F)(F)F)ccc1Cl ; CNc1ncc2c(n1)N1CCN=C1C(c1cc(NC(=O)Nc3cc(C(F)(F)F)ccc3Cl)c(F)cc1C)=C2]

### Step 1: Synthesis of 1-(2-chloro-5-(trifluoromethyl)phenyl)-3-(2-fluoro-4-methyl-5-(2-(methylamino)-8,9-dihydroimidazo[1′,2′:1,6]pyrido[2,3-d]pyrimidin-6-yl)phenyl)urea

To a mixture of 6-(5-amino-4-fluoro-2-methylphenyl)-N-methyl-8,9-dihydroimidazo[1′,2′:1,6]pyrido[2,3-d]pyrimidin-2-amine (40 mg, 0.12 mmol) and TEA (48 mg, 0.48 mmol) in THF (5 mL) was added 1-chloro-2-isocyanato-4-(trifluoromethyl)benzene (27 mg, 0.12 mmol). The reaction mixture was stirred at R.T under N2 for 3 hours. The mixture was concentrated and purified by prep-HPLC (0.1%/HCl/CH3CN/H2O) to afford 1-(2-chloro-5-(trifluoromethyl)phenyl)-3-(2-fluoro-4-methyl-5-(2-(methylamino)-8,9-dihydroimidazo[1′,2′:1,6]pyrido[2,3-d]pyrimidin-6-yl)phenyl)urea (13 mg, 18% yield) as a yellow solid. 1H NMR (400 MHz, DMSO-d6): δ 9.75 (d, J=6.4 Hz, 1H), 9.67 (s, 1H), 9.19 (s, 1H), 8.86 (s, 1H), 8.59 (d, J=2.0 Hz, 1H), 8.53-8.37 (m, 1H), 8.15 (d, J=8.4 Hz, 1H), 8.10 (s, 1H), 7.73 (d, J=8.8 Hz, 1H), 7.39-7.33 (m, 2H), 4.65-4.59 (m, 2H), 3.99-3.95 (m, 2H), 2.96 (d, J=4.8 Hz, 3H), 2.16 (s, 3H). LCMS (M+H⁺) m/z: 546.3.

### Example 336: Preparation of 1-(2-chlorophenyl)-3-(2-fluoro-4-methyl-3-(2-(methylamino)-8,9-dihydroimidazo[1′,2′:1,6]pyrido[2,3-d]pyrimidin-6-yl)phenyl)urea (Compound 336)

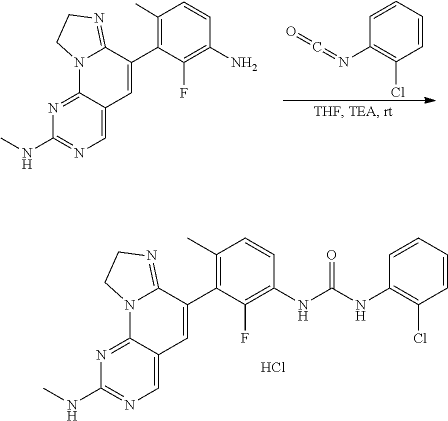 [SMILES: CNc1ncc2c(n1)N1CCN=C1C(c1c(C)ccc(N)c1F)=C2 ; O=C=Nc1ccccc1Cl ; CNc1ncc2c(n1)N1CCN=C1C(c1c(C)ccc(NC(=O)Nc3ccccc3Cl)c1F)=C2 ; Cl]

### Step 1: Synthesis of 1-(2-chlorophenyl)-3-(2-fluoro-4-methyl-3-(2-(methylamino)-8,9-dihydroimidazo[1′,2′:1,6]pyrido[2,3-d]pyrimidin-6-yl)phenyl)urea

To a mixture of 6-(3-amino-2-fluoro-6-methylphenyl)-N-methyl-8,9-dihydroimidazo[1′,2′:1,6]pyrido[2,3-d]pyrimidin-2-amine (30 mg, 0.092 mmol) and TEA (37 mg, 0.368 mmol) in THF (4 mL) was added 1-chloro-2-isocyanatobenzene (14 mg, 0.092 mmol). The reaction mixture was stirred at R.T under N2 for 16 hours. The mixture was concentrated and purified by prep-HPLC (0.1%/HCl/CH3CN/H2O) to afford 1-(2-chlorophenyl)-3-(2-fluoro-4-methyl-3-(2-(methylamino)-8,9-dihydroimidazo[1′,2′:1,6]pyrido[2,3-d]pyrimidin-6-yl)phenyl)urea (7 mg, 15% yield) as a yellow solid. ¹H NMR (400 MHz, DMSO-d₆): δ 9.94 (s, 1H), 9.42 (s, 1H), 8.88 (s, 2H), 8.61-8.58 (m, 1H), 8.24 (s, 1H), 8.19-8.12 (m, 2H), 7.48 (dd, J=8.0, 1.2 Hz, 1H), 7.32 (t, J=7.2 Hz, 1H), 7.20 (d, J=8.4 Hz, 1H), 7.09-7.05 (m, 1H), 4.73-4.55 (m, 2H), 4.07-4.01 (m, 2H), 2.98 (d, J=4.8 Hz, 3H), 2.17 (s, 3H). LCMS (M+H⁺) m/z: 478.1.

### Example 337: Preparation of 1-(4-chloro-2-fluorophenyl)-3-(2-fluoro-4-methyl-5-(2-(methylamino)-8,9-dihydroimidazo[1′,2′:1,6]pyrido[2,3-d]pyrimidin-6-yl)phenyl)urea (Compound 337)

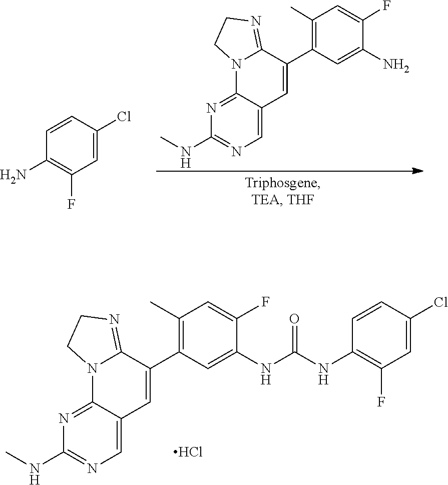

### Step 1: Synthesis of 1-(4-chloro-2-fluorophenyl)-3-(2-fluoro-4-methyl-5-(2-(methylamino)-8,9-dihydroimidazo[1′,2′:1,6]pyrido[2,3-d]pyrimidin-6-yl)phenyl)urea

A mixture of 4-chloro-2-fluoroaniline (23 mg, 0.16 mmol) in dry THE (20 mL) was added triphosgene (18 mg, 0.06 mmol) and TEA (0.1 mL). The reaction mixture stirred at 0° C. for 1 h and then 6-(5-amino-4-fluoro-2-methylphenyl)-N-methyl-8,9-dihydroimidazo[1′,2′:1,6]pyrido[2,3-d]pyrimidin-2-amine (50 mg, 0.15 mmol) was added. The reaction mixture was stirred at rt for 16 h. LCMS show the reaction was ok. The mixture was concentrated in vacuo. The residue was purified by prep-HPLC (0.1% HCl) to afford 1-(4-chloro-2-fluorophenyl)-3-(2-fluoro-4-methyl-5-(2-(methylamino)-8,9-dihydroimidazo[1′,2′:1,6]pyrido[2,3-d]pyrimidin-6-yl)phenyl)urea (23.7 mg, 32% yield) as a yellow solid. ¹H NMR (400 MHz, CD₃OD): δ 8.87 (s, 1H), 8.11-8.05 (m, 3H), 7.26-7.21 (m, 2H), 7.15-7.12 (m, 1H), 4.79-4.73 (m, 2H), 4.16 (t, J=10.0 Hz, 2H), 3.10 (s, 3H), 2.24 (s, 3H). LCMS (M+H⁺) m/z: 496.1.

### Example 338: Preparation of 1-(2-fluoro-4-methyl-5-(2-(methylamino)-8,9-dihydroimidazo[1′,2′:1,6]pyrido[2,3-d]pyrimidin-6-yl)phenyl)-3-(2-fluorophenyl)urea (Compound 338)

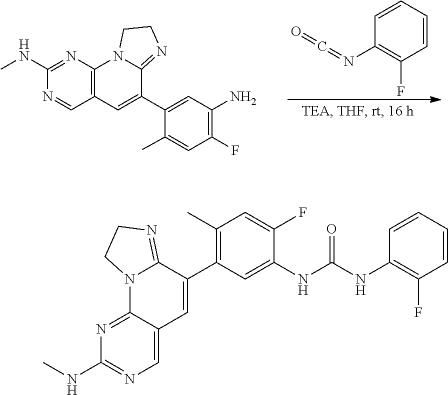 [SMILES: CNc1ncc2c(n1)N1CCN=C1C(c1cc(N)c(F)cc1C)=C2 ; O=C=Nc1ccccc1F ; CNc1ncc2c(n1)N1CCN=C1C(c1cc(NC(=O)Nc3ccccc3F)c(F)cc1C)=C2]

### Step 1: Synthesis of 1-(2-fluoro-4-methyl-5-(2-(methylamino)-8,9-dihydroimidazo[1′,2′:1,6]pyrido[2,3-d]pyrimidin-6-yl)phenyl)-3-(2-fluorophenyl)urea

To a solution of 6-(5-amino-4-fluoro-2-methylphenyl)-N-methyl-8,9-dihydroimidazo[1′,2′:1,6]pyrido[2,3-d]pyrimidin-2-amine (50 mg, 0.15 mmol) in THF (5 mL) was added TEA (0.5 mL) and 1-fluoro-2-isocyanatobenzene (41 mg, 0.3 mmol). The solvent was removed and then the residue was purified by prep-HPLC (0.1% HCl) to afford 1-(2-fluoro-4-methyl-5-(2-(methylamino)-8,9-dihydroimidazo[1′,2′:1,6]pyrido[2,3-d]pyrimidin-6-yl)phenyl)-3-(2-fluorophenyl)urea as (30 mg, 43% yield) a yellow solid. ¹H NMR (400 MHz, DMSO-d₆): δ 9.76 (s, 1H), 9.42 (s, 1H), 9.33 (s, 1H), 8.88 (s, 1H), 8.53-8.51 (m, 1H), 8.12-8.06 (m, 3H), 7.31 (d, J=12.0 Hz, 1H), 7.23 (t, J=9.6 Hz, 1H), 7.11 (t, J=7.6 Hz, 1H), 7.03-7.00 (m, 1H), 4.63-4.55 (m, 2H), 4.04-4.00 (m, 2H), 2.96 (s, 3H), 2.16 (s, 3H). LCMS (M+H⁺) m/z: 462.4.

### Example 339: Preparation of 1-(2-fluoro-4-methyl-5-(2-(methylamino)-8,9-dihydroimidazo[1′,2′:1,6]pyrido[2,3-d]pyrimidin-6-yl)phenyl)-3-(pyridin-2-yl)urea (Compound 339)

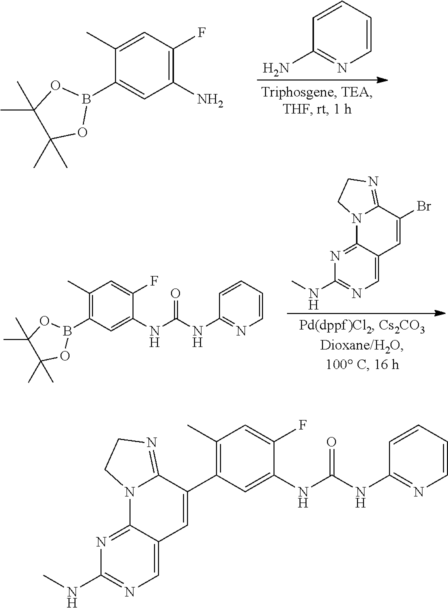

### Step 1: Synthesis of 1-(2-fluoro-4-methyl-5-(4, 4, 5, 5-tetramethyl-1, 3, 2-dioxaborolan-2-yl)phenyl)-3-(pyridin-2-yl) urea

To a mixture of 2-fluoro-4-methyl-5-(4,4,5,5-tetramethyl-1,3,2-dioxaborolan-2-yl)aniline (130 mg, 0.52 mmol) in THF (20 mL) was added triphosgene (62 mg, 0.21 mmol) at 0° C. slowly. The mixture was stirred at 0° C.-rt for 0.5 h under N2. Then pyridin-2-amine (49 mg, 0.52 mmol) and TEA (157 mg, 1.56 mmol) was added. The mixture was stirred at rt for 0.5 h under N2. LCMS show the reaction was OK. Concentration in vacuum and purification by silica gel column (PE/EA=2:1) gave 1-(2-fluoro-4-methyl-5-(4,4,5,5-tetramethyl-1,3,2-dioxaborolan-2-yl)phenyl)-3-(pyridin-2-yl)urea (40 mg, 21.1% yield) as a yellow solid.

### Step 2: Synthesis of 1-(2-fluoro-4-methyl-5-(2-(methylamino)-8,9-dihydroimidazo[1′,2′:1,6]pyrido[2,3-d]pyrimidin-6-yl)phenyl)-3-(pyridin-2-yl)urea

A mixture of 1-(2-fluoro-4-methyl-5-(4,4,5,5-tetramethyl-1,3,2-dioxaborolan-2-yl)phenyl)-3-(pyridin-2-yl)urea (40 mg, 0.11 mmol) and 6-bromo-N-methyl-8,9-dihydroimidazo[1′,2′:1,6]pyrido[2,3-d]pyrimidin-2-amine (30 mg, 0.11 mmol) in dioxane/H₂O (5 mL/1 mL) was added Cs2CO3 (105 mg, 0.33 mmol) and Pd(dppf)Cl₂ (19 mg, 0.022 mmol), the mixture was stirred at 100° C. under N2 overnight. The mixture concentrated in vacuum, purified by prep-HPLC (0.1% NH4CO3) to afford 1-(2-fluoro-4-methyl-5-(2-(methylamino)-8,9-dihydroimidazo[1′,2′:1,6]pyrido[2,3-d]pyrimidin-6-yl)phenyl)-3-(pyridin-2-yl)urea (13.8 mg, 28.3% yield) as a yellow solid. ¹H NMR (400 MHz, DMSO-d6): δ 10.99 (s, 1H), 9.79 (s, 1H), 8.27-8.23 (m, 2H), 8.04 (d, J=8.0 Hz, 1H), 7.78-7.74 (m, 1H), 7.48-7.35 (m, 2H), 7.17 (d, J=12.0 Hz, 2H), 7.04-7.01 (m, 1H), 4.11-3.89 (m, 4H), 2.84 (s, 3H), 2.17 (s, 3H). LCMS (M+H+) m/z: 445.0.

### Example 340: Preparation of N-(3-fluoro-4-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (Compound 340)

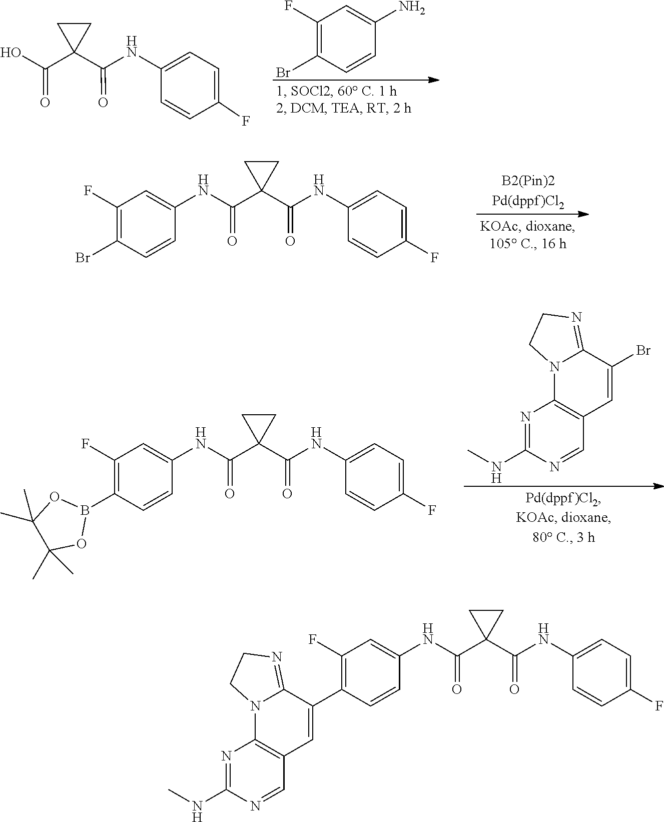

### Step 1: Synthesis of N-(4-bromo-3-fluorophenyl)-N-(4-fluorophenyl)cyclopropane-1,1-dicarboxamide

The mixture of 1-((4-fluorophenyl)carbamoyl)cyclopropane-1-carboxylic acid (223 mg, 1.0 mmol) in SOCl₂ (3 mL) was stirred for 2 h at 70° C. The reaction mixture was concentrated and a solution of 4-bromo-3-fluoroaniline (200 mg, 1.1 mmol) and TEA (500 mg 5.0 mmol) in DCM (3 mL) were added to reaction mixture. The reaction mixture was stirred at 25° C. for 3 h. Concentration and purification by Flash (PE:EA=3:1˜1:1) gave N-(4-bromo-3-fluorophenyl)-N-(4-fluorophenyl)cyclopropane-1,1-dicarboxamide (180 mg) as white solid. LCMS (M+H⁺) m/z: 394.9.

### Step 2: Synthesis of N-(3-fluoro-4-(4,4,5,5-tetramethyl-1,3,2-dioxaborolan-2-yl)phenyl)-N-(4-fluorophenyl)cyclopropane-1,1-dicarboxamide

The mixture of N-(4-bromo-3-fluorophenyl)-N-(4-fluorophenyl)cyclopropane-1,1-dicarboxamide (147 mg, 0.5 mmol, 1.0 equiv), Pin₂B₂ (190 mg, 0.75 mmol, 1.5 equiv), KOAc (150 mg, 1.5 mmol, 3.0 equiv), and PdCl₂ (dppf) (73 mg, 0.1 mmol, 20 mol %) in 1, 4-dioxane (4 mL) was refluxed for 16 h under Ar. The reaction mixture was used for next step without further purification. LCMS (M+H⁺) m/z: 443.0

### Step 3: Synthesis of N-(3-fluoro-4-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide

The mixture of N-(3-fluoro-4-(4,4,5,5-tetramethyl-1,3,2-dioxaborolan-2-yl)phenyl)-N-(4-fluorophenyl)cyclopropane-1,1-dicarboxamide (crude, 0.2 mmol, 1 equiv), 6-bromo-N-methyl-8,9-dihydroimidazo[1′,2′:1,6]pyrido[2,3-d]pyrimidin-2-amine (56 mg, 0.2 mmol, 1.0 equiv), K₂CO₃ (82 mg, 0.6 mmol, 3.0 equiv), and PdCl₂ (dppf) (29 mg, 0.04 mmol, 20 mol %) in 1, 4-dioxane (4.0 mL) and H₂O (1.0 mL) was stirred at 85° C. for 2 h under Ar. The reaction mixture was purified by HPLC (NH₄HCO₃) to give N-(3-fluoro-4-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (11.7 mg) as white solid. ¹H NMR (400 MHz, DMSO-d₆): δ 10.31 (s, 1H), 10.00 (s, 1H), 8.26-8.20 (m, 1H), 7.69-7.61 (m, 3H), 7.55 (t, J=8.4 Hz, 1H), 7.46-7.44 (m, 1H), 7.36 (d, J=8.0 Hz, 1H), 7.27 (s, 1H), 7.15 (t, J=8.8 Hz, 2H), 4.04-3.92 (m, 4H), 2.83 (s, 3H), 1.46-1.44 (m, 4H). LCMS (M+H⁺) m/z: 516.0.

### Example 341: Preparation of N-(4-fluoro-3-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (Compound 341)

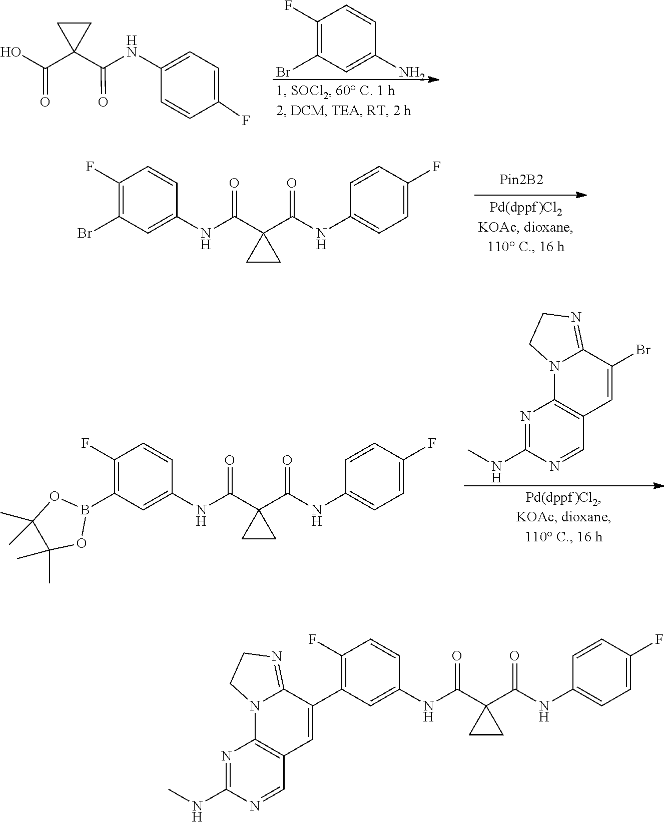

### Step 1: Synthesis of N-(3-bromo-4-fluorophenyl)-N-(4-fluorophenyl)cyclopropane-1,1-dicarboxamide

A solution of 1-((4-fluorophenyl)carbamoyl)cyclopropane-1-carboxylic acid (200 mg, 0.6 mmol) in SOCl₂ (3 mL) was stirred at 60° C. for 1 h. The reaction mixture was concentrated to obtain a crude solid. A solution of 3-bromo-4-fluoroaniline (171 mg, 0.9 mmol) and TEA (182 mg, 1.8 mmol) in DCM (5 mL) was added. The reaction mixture was stirred at r.t. for 2 h, LCMS showed the reaction completed. The reaction mixture was diluted to water (10 mL), extracted by DCM (10 mL*3). The combined organic phase was washed by brine (30 mL), dried over anhydrous Na₂SO₄, concentrated to obtain a white solid, which was purified by chromatography column (PE:EA=4:1) to afford N-(3-bromo-4-fluorophenyl)-N-(4-fluorophenyl)cyclopropane-1,1-dicarboxamide (300 mg, 84% yield). LCMS (M+H⁺) m/z: 394.8

### Step 2: Synthesis of N-(4-fluoro-3-(4,4,5,5-tetramethyl-1,3,2-dioxaborolan-2-yl)phenyl)-N-(4-fluorophenyl)cyclopropane-1,1-dicarboxamide

To a solution of N-(3-bromo-4-fluorophenyl)-N-(4-fluorophenyl)cyclopropane-1,1-dicarboxamide (290 mg, 0.73 mmol) in dioxane (5 mL) was added Pin2B2 (185 mg, 0.73 mmol), KOAc (142 mg, 1.46 mmol) and Pd(dppf)Cl₂ (51 mg, 0.07 mmol) under nitrogen atmosphere. The mixture was stirred at 110° C. for 18 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by chromatography column (PE:EA=2:1) to afford N-(4-fluoro-3-(4,4,5,5-tetramethyl-1,3,2-dioxaborolan-2-yl)phenyl)-N-(4-fluorophenyl)cyclopropane-1,1-dicarboxamide (170 mg, 53% yield) as white solid. LCMS (M+H⁺) m/z: 443.0

### Step 3: Synthesis of N-(4-fluoro-3-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide

To a solution of N-(4-fluoro-3-(4,4,5,5-tetramethyl-1,3,2-dioxaborolan-2-yl)phenyl)-N-(4-fluorophenyl)cyclopropane-1,1-dicarboxamide (41 mg, 0.1 mmol) in dioxane (4 mL) and H₂O (1 mL) was added 6-bromo-N-methyl-8,9-dihydroimidazo[1′,2′:1,6]pyrido[2,3-d]pyrimidin-2-amine (27 mg, 0.1 mmol), K₂CO₃ (28 mg, 0.2 mmol) and Pd(dppf)Cl₂ (7 mg, 0.01 mmol) under nitrogen atmosphere. The mixture was stirred at 90° C. for 6 h. LCMS showed the reaction completed. The mixture was diluted to water (10 mL), extracted by EA (10 mL*3). The combined organic phase was washed by brine (20 mL), dried over anhydrous Na₂SO₄, filtered and concentrated to afford a crude solid, which was purified by chromatography column (PE:EA=1:2) to afford N-(4-fluoro-3-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (4.5 mg, 8% yield) as yellow solid. ¹H NMR (400 MHz, CD₃OD): δ 8.44-8.41 (m, 1H), 8.17 (s, 1H), 7.84-7.81 (m, 1H), 7.68-7.65 (m, 1H), 7.58-7.55 (m, 2H), 7.31 (t, J=9.2 Hz, 1H), 7.09 (t, J=8.8 Hz, 2H), 4.80-4.65 (m, 2H), 4.15 (t, J=10.0 Hz, 2H), 3.09 (s, 3H), 1.66-1.63 (m, 4H). LCMS (M+H⁺) m/z: 516.0.

### Example 342: Preparation of N-(4-chloro-3-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (Compound 342)

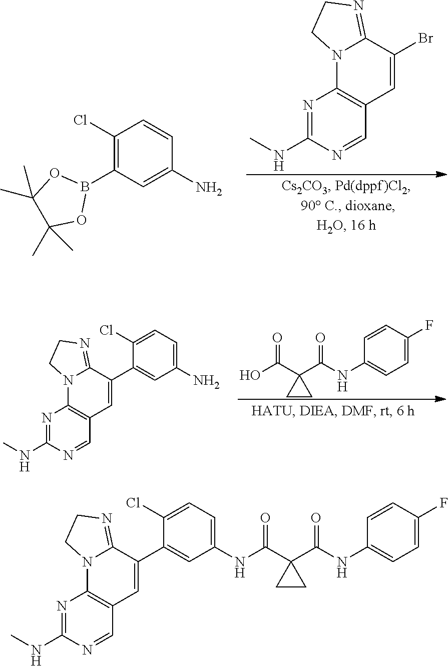 [SMILES: CC1(C)OB(c2cc(N)ccc2Cl)OC1(C)C ; CNc1ncc2c(n1)N1CCN=C1C(Br)=C2 ; CNc1ncc2c(n1)N1CCN=C1C(c1cc(N)ccc1Cl)=C2 ; O=C(O)C1(C(=O)Nc2ccc(F)cc2)CC1 ; CNc1ncc2c(n1)N1CCN=C1C(c1cc(NC(=O)C3(C(=O)Nc4ccc(F)cc4)CC3)ccc1Cl)=C2]

### Step 1: Synthesis of 6-(5-amino-2-chlorophenyl)-N-methyl-8,9-dihydroimidazo[1′,2′:1,6]pyrido[2,3-d]pyrimidin-2-amine

A mixture of 4-chloro-3-(4,4,5,5-tetramethyl-1,3,2-dioxaborolan-2-yl)aniline (65 mg, 0.26 mmol), CsCO₃ (9.5 g, 97 mmol), and Pd(dppf)Cl₂ (8 mg, 0.01 mmol) in dioxane (10 mL) and H₂O (1 mL) was degassed and charged with N₂ three times and stirred at 90° C. for 16 h under N₂. The reaction mixture was concentrated and purified by Prep-HPLC (0.1% NH₃·H₂O) to afford 6-(5-amino-2-chlorophenyl)-N-methyl-8,9-dihydroimidazo[1′,2′:1,6]pyrido[2,3-d]pyrimidin-2-amine (50 mg, 59% yield) as yellow solid. LCMS (M+H⁺) m/z: 327.1.

### Step 2: Synthesis of N-(4-chloro-3-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide

To a solution of 1-((4-fluorophenyl)carbamoyl)cyclopropane-1-carboxylic acid (45 mg, 0.20 mmol), 6-(5-amino-2-chlorophenyl)-N-methyl-8,9-dihydroimidazo[1′,2′:1,6]pyrido[2,3-d]pyrimidin-2-amine (60 mg, 0.18 mmol), and HATU (105 mg, 0.28 mmol) in DMF (5 mL) was added DIEA (70 mg, 0.55 mmol). The resulting mixture was stirred at r.t. for 16 h under N₂. The reaction mixture was added into water (40 mL) slowly, stirred at r.t. for 30 min, filtered. The collected cake was purified by Prep-HPLC (0.1% NH₃·H₂O) to afford N-(4-chloro-3-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (5.5 mg, 5.6% yield) as a yellow solid. ¹H NMR (400 MHz, DMSO-d₆): δ 10.17 (s, 1H), 9.99 (s, 1H), 8.24-8.22 (m, 1H), 7.70-7.68 (m, 1H), 7.66-7.61 (m, 3H), 7.46-7.40 (m, 2H), 7.19-7.11 (m, 3H), 4.07-3.85 (m, 4H), 2.84 (s, 3H), 1.48-1.42 (m, 4H). LCMS (M+H⁺) m/z: 532.2.

### Example 343: Preparation of N-(2-fluoro-4-methyl-5-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (Compound 343)

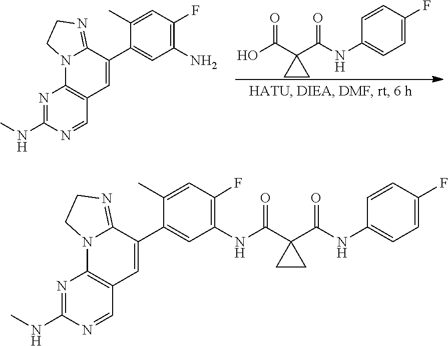 [SMILES: CNc1ncc2c(n1)N1CCN=C1C(c1cc(N)c(F)cc1C)=C2 ; O=C(O)C1(C(=O)Nc2ccc(F)cc2)CC1 ; CNc1ncc2c(n1)N1CCN=C1C(c1cc(NC(=O)C3(C(=O)Nc4ccc(F)cc4)CC3)c(F)cc1C)=C2]

The preparation of 6-(5-amino-4-fluoro-2-methylphenyl)-N-methyl-8,9-dihydroimidazo[1′,2′:1,6]pyrido[2,3-d]pyrimidin-2-amine was described in Example 139

### Step 1: Synthesis of N-(2-fluoro-4-methyl-5-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide

To a mixture of 1-((4-fluorophenyl)carbamoyl)cyclopropane-1-carboxylic acid (55 mg, 0.25 mmol), 6-(5-amino-4-fluoro-2-methylphenyl)-N-methyl-8,9-dihydroimidazo[1′,2′:1,6]pyrido[2,3-d]pyrimidin-2-amine (80 mg, 0.25 mmol), and HATU (190 mg, 0.50 mmol) in DMF (5.0 mL) was added DIEA (65 mg, 0.50 mmol). The resulting mixture was stirred at r.t. for 16 h under N₂. The reaction mixture was quenched with water (20 mL) and extracted with DCM (20 mL×3). The combined organic layers were washed with brine (20 mL), dried over Na₂SO₄, filtered, concentrated to afford crude product, which was purified by Pre-HPLC (0.1% NH₃·H₂O) to afford N-(2-fluoro-4-methyl-5-(2-(methylamino)-8,9-dihydroimidazo[1′,2′:1,6]pyrido[2,3-d]pyrimidin-6-yl)phenyl)-N-(4-fluorophenyl)cyclopropane-1,1-dicarboxamide (21.7 mg, 16.5% yield) as a yellow solid. ¹H NMR (400 MHz, DMSO-d₆): δ 10.51 (s, 1H), 9.93 (s, 1H), 8.21-8.17 (m, 1H), 7.71 (d, J=7.2 Hz, 1H), 7.61-7.57 (m, 2H), 7.40-7.39 (m, 1H), 7.18-7.13 (m, 3H), 7.07 (s, 1H), 4.00-3.95 (m, 2H), 3.89 (t, J=8.8 Hz, 2H), 2.83 (d, J=4.0 Hz, 3H), 2.18 (s, 3H), 1.58-1.54 (m, 4H). LCMS (M+H⁺) m/z: 530.3.

### Example 344: Preparation of N-(2-fluoro-4-methyl-5-(2-(oxetan-3-ylamino)-8,9-dihydroimidazo[1′,2′:1,6]pyrido[2,3-d]pyrimidin-6-yl)phenyl)-N-(4-fluorophenyl)cyclopropane-1,1-dicarboxamide (Compound 344)

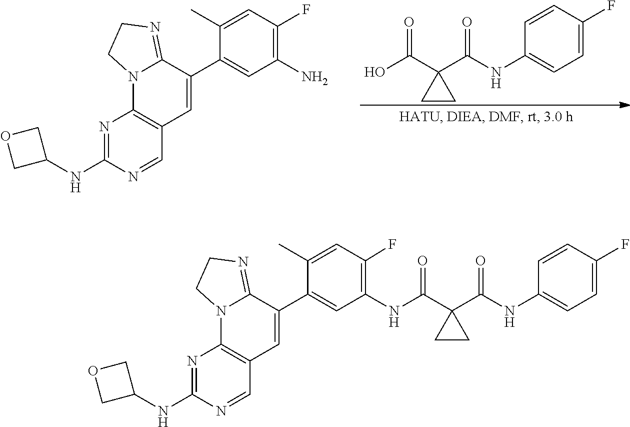 [SMILES: Cc1cc(F)c(N)cc1C1=Cc2cnc(NC3COC3)nc2N2CCN=C12 ; O=C(O)C1(C(=O)Nc2ccc(F)cc2)CC1 ; Cc1cc(F)c(NC(=O)C2(C(=O)Nc3ccc(F)cc3)CC2)cc1C1=Cc2cnc(NC3COC3)nc2N2CCN=C12]

The preparation of 6-(5-amino-4-fluoro-2-methylphenyl)-N-methyl-8,9-dihydroimidazo[1′,2′:1,6]pyrido[2,3-d]pyrimidin-2-amine was described in Example 178

### Step 1: Synthesis of N-(2-fluoro-4-methyl-5-(2-(oxetan-3-ylamino)-8,9-dihydroimidazo[1′,2′:1,6]pyrido[2,3-d]pyrimidin-6-yl)phenyl)-N-(4-fluorophenyl)cyclopropane-1,1-dicarboxamide

To a solution of 1-((4-fluorophenyl)carbamoyl)cyclopropane-1-carboxylic acid (36.6 mg, 0.164 mmol), 6-(5-amino-4-fluoro-2-methylphenyl)-N-(oxetan-3-yl)-8,9-dihydroimidazo[1′,2′:1,6]pyrido[2,3-d]pyrimidin-2-amine (60 mg, 0.164 mmol) and HATU (125 mg, 0.328 mmol) in DMF (4.0 mL), was added DIEA (42 mg, 0.328 mmol). The resulting mixture was stirred at r.t. for 3 h under N₂. The reaction mixture was added into water (40 mL), extracted with DCM (30 mL×3). The combined organic phase was washed with brine, dried over Na₂SO₄, filtered and concentrated. The residue was purified by column chromatography on column chromatography (DCM:MeOH=12:1, +0.1% NH₃-MeOH), and then triturated with CH₃CN (5.0 mL) to afford N-(2-fluoro-4-methyl-5-(2-(oxetan-3-ylamino)-8,9-dihydroimidazo[1′,2′:1,6]pyrido[2,3-d]pyrimidin-6-yl)phenyl)-N-(4-fluorophenyl)cyclopropane-1,1-dicarboxamide (32.6 mg, 34.7% yield) as a yellow solid. ¹H NMR (400 MHz, DMSO-d₆): δ 10.53 (s, 1H), 9.92 (s, 1H), 8.21 (s, 2H), 7.70 (d, J=7.6 Hz, 1H), 7.60-7.57 (m, 2H), 7.18-7.09 (m, 4H), 4.94-4.91 (m, 1H), 4.75 (t, J=6.0 Hz, 2H), 4.53 (t, J=6.0 Hz, 2H), 4.02-3.98 (m, 2H), 3.92-3.87 (m, 2H), 2.17 (s, 3H), 1.61-1.51 (m, 4H). LCMS (M+H⁺) m/z: 572.3.

### Example 345: Preparation of N-(4-fluorophenyl)-N-(4-((2-(methylamino)-8,9-dihydroimidazo[1′,2′:1,6]pyrido[2,3-d]pyrimidin-6-yl)oxy)phenyl)cyclopropane-1,1-dicarboxamide (Compound 345)

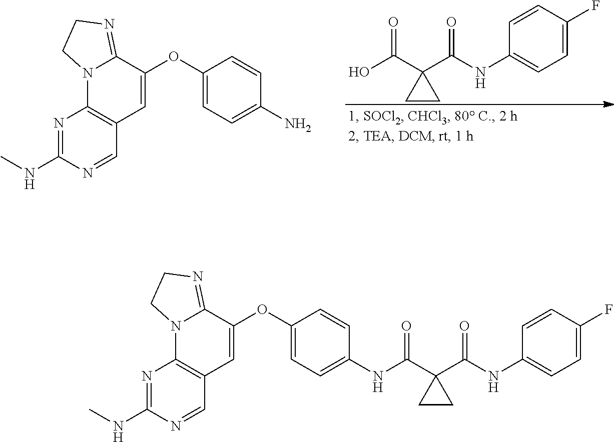 [SMILES: CNc1ncc2c(n1)N1CCN=C1C(Oc1ccc(N)cc1)=C2 ; O=C(O)C1(C(=O)Nc2ccc(F)cc2)CC1 ; CNc1ncc2c(n1)N1CCN=C1C(Oc1ccc(NC(=O)C3(C(=O)Nc4ccc(F)cc4)CC3)cc1)=C2]

### Step 1: Synthesis of N-(4-fluorophenyl)-N-(4-((2-(methylamino)-8,9-dihydroimidazo[1′,2′:1,6]pyrido[2,3-d]pyrimidin-6-yl)oxy)phenyl)cyclopropane-1,1-dicarboxamide

To a mixture of 1-((4-fluorophenyl)carbamoyl)cyclopropane-1-carboxylic acid (223 mg, 1.0 mmol) in CHCl₃ (1.0 mL) was added SOCl₂ (1.0 mL) at rt. The reaction mixture was stirred for 2 h at 80° C. The reaction was concentrated to give crude 1-((4-fluorophenyl)carbamoyl)cyclopropane-1-carbonyl chloride. To a mixture of 6-(4-aminophenoxy)-N-methyl-8,9-dihydroimidazo[1′,2′:1,6]pyrido[2,3-d]pyrimidin-2-amine (60 mg, 0.2 mmol), DIPEA (78 mg, 0.6 mmol) in DCM (5.0 mL), was added a solution of crude 1-((4-fluorophenyl)carbamoyl)cyclopropane-1-carbonyl chloride (58 mg, 0.24 mmol) in DCM (1.0 mL). The reaction was stirred for 2 h at 25° C. The reaction was concentrated and purified by HPLC (NH₄HCO₃) to give N-(4-fluorophenyl)-N-(4-((2-(methylamino)-8,9-dihydroimidazo[1′,2′:1,6]pyrido[2,3-d]pyrimidin-6-yl)oxy)phenyl)cyclopropane-1,1-dicarboxamide (47.1 mg) as white solid. ¹H NMR (400 MHz, DMSO-d₆): δ 10.05 (d, J=17.2 Hz, 2H), 8.14 (s, 1H), 7.65-7.58 (m, 4H), 7.31-7.29 (m, 1H), 7.17-7.12 (m, 2H), 7.04-7.00 (m, 2H), 6.80-6.75 (m, 1H), 4.04-3.92 (m, 4H), 2.81 (s, 3H), 1.45 (s, 4H). LCMS (M+H⁺) m/z: 514.0.

### Example 346: Preparation of N-(3-fluoro-4-((2-(methylamino)-8,9-dihydroimidazo[1′,2′:1,6]pyrido[2,3-d]pyrimidin-6-yl)oxy)phenyl)-N-(4-fluorophenyl)cyclopropane-1,1-dicarboxamide (Compound 346)

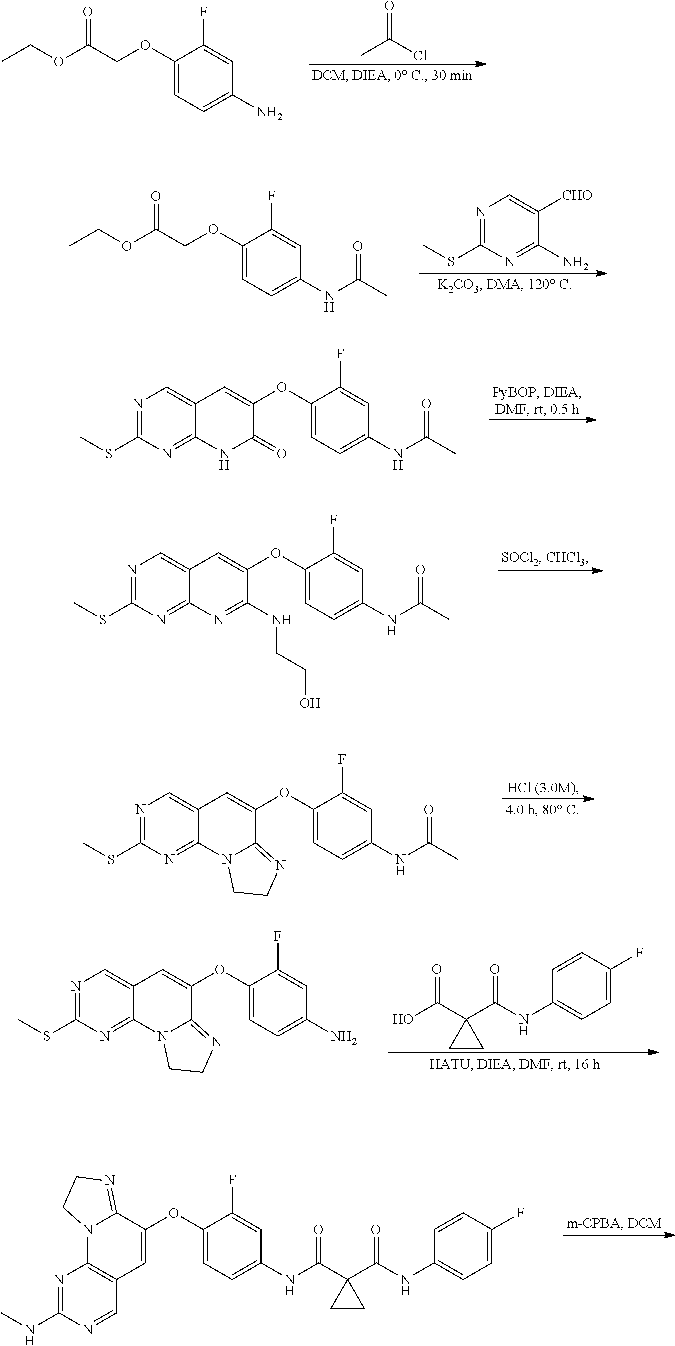

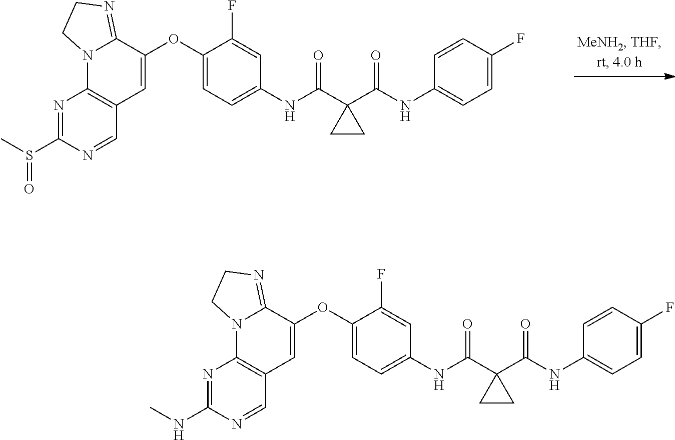 [SMILES: CS(=O)c1ncc2c(n1)N1CCN=C1C(Oc1ccc(NC(=O)C3(C(=O)Nc4ccc(F)cc4)CC3)cc1F)=C2 ; CNc1ncc2c(n1)N1CCN=C1C(Oc1ccc(NC(=O)C3(C(=O)Nc4ccc(F)cc4)CC3)cc1F)=C2]

### Step 1: Synthesis of ethyl 2-(4-acetamido-2-fluorophenoxy)acetate

To a mixture of ethyl 2-(4-amino-2-fluorophenoxy)acetate (3.65 g, 17.14 mmol) and DIEA (6.63 g, 51.42 mmol) in dry DCM (40 mL) was added acetyl chloride (2.02 g, 25.71 mmol) drop-wise at 0° C. under N₂. The resulting mixture was stirred at 0° C. for 30 min, quenched with sat. NaHCO₃ (30 mL) and extracted with DCM (50.0 mL×2). The combined organic phase was washed with brine (50.0 mL), dried over Na₂SO₄, filtered and concentrated. The residue was purified by column chromatography on silica gel (PE:EA=1:1) to afford ethyl 2-(4-acetamido-2-fluorophenoxy)acetate (4.0 g, 91.5% yield) as an off-white solid. LCMS (M+H⁺) m/z: 256.2.

### Step 2: Synthesis of N-(3-fluoro-4-((2-(methylthio)-7-oxo-7,8-dihydropyrido[2,3-d]pyrimidin-6-yl)oxy)phenyl)acetamide

A mixture of 4-amino-2-(methylthio)pyrimidine-5-carbaldehyde (973 mg, 5.76 mmol), ethyl 2-(4-acetamido-2-fluorophenoxy)acetate (1.64 g, 6.04 mmol) and K₂CO₃ (2.39 g, 17.28 mmol) in DMA (40 mL) was stirred at 120° C. for 16 h under N₂. The reaction mixture was cooled to r.t and added into water (400 mL), pH was adjusted to 3.0˜4.0 by hydrochloric acid (3M). the mixture was stirred at r.t for 30 min, filtered. The collected cake was washed with water (100 mL), triturated with CH₃CN (10 mL) to afford N-(3-fluoro-4-((2-(methylthio)-7-oxo-7,8-dihydropyrido[2,3-d]pyrimidin-6-yl)oxy)phenyl)acetamide (1.26 g, 60.8% yield) as a brown solid. LCMS (M+H⁺) m/z: 360.9.

### Step 3: Synthesis of N-(3-fluoro-4-((7-((2-hydroxyethyl)amino)-2-(methylthio)pyrido[2,3-d]pyrimidin-6-yl)oxy)phenyl)acetamide

To a mixture of N-(3-fluoro-4-((2-(methylthio)-7-oxo-7,8-dihydropyrido[2,3-d]pyrimidin-6-yl)oxy)phenyl)acetamide (1.35 g, 3.75 mmol), 2-aminoethan-1-ol (422 mg, 5.62 mmol) and PyBOP (2.92 g, 5.62 mmol) in DMF (20 mL) was added DIEA (967 mg, 7.50 mmol). The resulting mixture was stirred at r.t for 0.5 h under N₂. The reaction mixture was added into water (200 mL), stirred at r.t for 30 min, filtered. The collected cake was washed with water (50 mL), triturated with CH₃CN (15 mL) to afford N-(3-fluoro-4-((7-((2-hydroxyethyl)amino)-2-(methylthio)pyrido[2,3-d]pyrimidin-6-yl)oxy)phenyl)acetamide (1.41 g, 93.4% yield) as a gray solid. LCMS (M+H⁺) m/z: 404.1

### Step 4: Synthesis of N-(3-fluoro-4-((2-(methylthio)-8,9-dihydroimidazo[1′,2′:1,6]pyrido[2,3-d]pyrimidin-6-yl)oxy)phenyl)acetamide

To a mixture of N-(3-fluoro-4-((7-((2-hydroxyethyl)amino)-2-(methylthio)pyrido[2,3-d]pyrimidin-6-yl)oxy)phenyl)acetamide (1.41 g, 3.50 mmol) in CHCl₃ (35.0 mL) was added SOCl₂ (2.08 g, 17.50 mmol). The resulting mixture was stirred at 70° C. for 16 h under N₂. The reaction mixture was concentrated and added into sat. NaHCO₃ (50.0 mL), stirred at r.t for 30 min, filtered. The collected cake was washed with water (50 mL), triturated with CH₃CN (30 mL) to afford N-(3-fluoro-4-((2-(methylthio)-8,9-dihydroimidazo[1′,2′:1,6]pyrido[2,3-d]pyrimidin-6-yl)oxy)phenyl)acetamide (1.235 g, 91.7% yield) as a gray solid. LCMS (M+H⁺) m/z: 386.1

### Step 5: Synthesis of 3-fluoro-4-((2-(methylthio)-8,9 dihydroimidazo[1′,2′:1,6]pyrido[2,3-d]pyrimidin-6-yl)oxy)aniline

A mixture of N-(3-fluoro-4-((2-(methylthio)-8,9-dihydroimidazo[1′,2′:1,6]pyrido[2,3-d]pyrimidin-6-yl)oxy)phenyl)acetamide (1.235 g, 3.21 mmol) in aq. HCl (3.0 M, 40.0 mL) was stirred at 80° C. for 4 h under N₂. The reaction mixture was cooled to 0° C., and aq. NaOH (4.0 M) was added to pH=7-8, stirred at r.t for 30 min, filtered. The collected cake was washed with water (50 mL), triturated with CH₃CN (15 mL) to afford 3-fluoro-4-((2-(methylthio)-8,9-dihydroimidazo[1′,2′:1,6]pyrido[2,3-d]pyrimidin-6-yl)oxy)aniline (700 mg, 63.6% yield) as a gray solid. LCMS (M+H⁺) m/z: 344.1.

### Step 6: Synthesis of N-(3-fluoro-4-((2-(methylthio)-8,9-dihydroimidazo[1′,2′:1,6]pyrido[2,3-d]pyrimidin-6-yl)oxy)phenyl)-N-(4-fluorophenyl)cyclopropane-1,1-dicarboxamide

To a mixture of 3-fluoro-4-((2-(methylthio)-8,9-dihydroimidazo[1′,2′:1,6]pyrido[2,3-d]pyrimidin-6-yl)oxy)aniline (80 mg, 0.233 mmol), 1-((4-fluorophenyl)carbamoyl)cyclopropane-1-carboxylic acid (52 mg, 0.233 mmol), HATU (133 mg, 0.350 mmol) in DMF (4.0 mL) was added DIEA (60 mg, 0.466 mmol). The resulting mixture was stirred at r.t for 16 h under N₂. The reaction mixture was added to water (40 mL), stirred at r.t for 30 min, filtered. The collected cake was purified by column chromatography on column chromatography (DCM/MeOH=20/1) to afford N-(3-fluoro-4-((2-(methylthio)-8,9-dihydroimidazo[1′,2′:1,6]pyrido[2,3-d]pyrimidin-6-yl)oxy)phenyl)-N-(4-fluorophenyl)cyclopropane-1,1-dicarboxamide (80 mg, 62.5% yield) as a brown solid. LCMS (M+H⁺) m/z: 549.1.

### Step 7: Synthesis of N-(3-fluoro-4-((2-(methylsulfinyl)-8,9-dihydroimidazo[1′,2′:1,6]pyrido[2,3-d]pyrimidin-6-yl)oxy)phenyl)-N-(4-fluorophenyl)cyclopropane-1,1-dicarboxamide

To a mixture of N-(3-fluoro-4-((2-(methylthio)-8,9-dihydroimidazo[1′,2′:1,6]pyrido[2,3-d]pyrimidin-6-yl)oxy)phenyl)-N-(4-fluorophenyl)cyclopropane-1,1-dicarboxamide (80 mg, 0.146 mmol) in DCM (5.0 mL) was added m-CPBA (64 mg, 0.438 mmol) at 0° C. The resulting mixture was stirred at 0° C. for 0.5 h. The reaction mixture was concentrated to afford crude N-(3-fluoro-4-((2-(methylsulfinyl)-8,9-dihydroimidazo[1′,2′:1,6]pyrido[2,3-d]pyrimidin-6-yl)oxy)phenyl)-N-(4-fluorophenyl)cyclopropane-1,1-dicarboxamide (144 mg) as a yellow solid. LCMS (M+H⁺) m/z: 565.1

### Step 8: Synthesis of N-(3-fluoro-4-((2-(methylamino)-8,9-dihydroimidazo[1′,2′:1,6]pyrido[2,3-d]pyrimidin-6-yl)oxy)phenyl)-N-(4-fluorophenyl)cyclopropane-1,1-dicarboxamide

To a mixture of crude N-(3-fluoro-4-((2-(methylsulfinyl)-8,9-dihydroimidazo[1′,2′:1,6]pyrido[2,3-d]pyrimidin-6-yl)oxy)phenyl)-N-(4-fluorophenyl)cyclopropane-1,1-dicarboxamide (144 mg, 0.146 mmol) in THF (2.5 mL) was added Me-NH₂ (2.0 M, 0.37 mL, 0.73 mmol). The resulting mixture was stirred at rt for 4.0 h. The reaction mixture was poured into water (30 mL) and extracted with DCM (50 mL×3). The combined organic phase was washed with brine (50 mL), dried over Na₂SO₄, filtered and concentrated. The residue was purified by column chromatography on silica gel (DCM/MeOH=10:1), followed triturated with EA (5.0 mL) to afford N-(3-fluoro-4-((2-(methylamino)-8,9-dihydroimidazo[1′,2′:1,6]pyrido[2,3-d]pyrimidin-6-yl)oxy)phenyl)-N-(4-fluorophenyl)cyclopropane-1,1-dicarboxamide (31.3 mg, 23.1% yield over 2 steps) as a yellow solid. ¹H NMR (400 MHz, DMSO-d₆): δ 10.23 (s, 1H), 10.00 (s, 1H), 8.11 (s, 1H), 7.77 (dd, J=13.6, 2.4 Hz, 1H), 7.64-7.61 (m, 2H), 7.35 (d, J=9.2 Hz, 1H), 7.24-7.23 (m, 1H), 7.19-7.12 (m, 3H), 6.65 (s, 1H), 4.36-4.33 (m, 4H), 2.79 (d, J=4.0 Hz, 3H), 1.45-1.44 (m, 4H). LCMS (M+H⁺) m/z: 532.3.

While the foregoing written description of the compounds, uses, and methods described herein enables one of ordinary skill to make and use the compounds, uses, and methods described herein, those of ordinary skill will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

All references disclosed herein are incorporated by reference in their entirety.

The in vitro and in vivo activities of the compounds of Formula (I) were determined using the following procedures.

### Biological Example B1

In Vitro Kinase Assays

The effect of the compounds on the activity of SRC and PAK1 kinase were assessed. SRC and PAK1 kinase enzymes were obtained from Carna Biosciences.

Test compounds were dissolved in 100% DMSO to prepare a 20 mM stock. Compounds were stored in −20° C. and protected from light. A 100×solution of the compounds were prepared, from which 4-fold serial dilutions was made to achieve a total of 6 concentrations. Saracatinib was used as a positive control for the SRC assay, and FRAX597 was used as a positive control for the PAK1 assay. For the positive controls, a 3-fold serial dilution was made to achieve a total of 10 concentrations. 10 μL 1× Kinase buffer was used as the negative control.

First, 250 nL of the compounds at each dilution were transferred into wells of a 384-well plate. The PAK1 and SRC kinases were diluted to a 2.5×final concentration with 1×Kinase buffer. Then, 10 μL of enzyme mix was added to the 384-well plate, and the enzymes and compounds were pre-incubated at room temperature for 10 minutes. The substrate mix containing ATP and Kinase substrate at 1.67× final concentration was prepared with 1×Kinase buffer. Then, 15 μL of substrate mix was added to a 384-well plate and reacted at room temperature for 30 min and 60 min. The reaction was stopped by adding 30 μL of the stop buffer, and the conversion rate was read using a Caliper EZ Reader.

Percent inhibition is calculated according to the following equation:

$$\% inhibition = \frac{[ Conversion \% \_max - Conversion \% \_sample ]}{[ Conversion \% \_max - Conversion \% \_min ]} \times 100$$

wherein “Conversion %_sample” is the conversion percentage value of the sample; “Conversion %_min” is the average conversion percentage value of the negative control; and “Conversion %_max” is the average conversion percentage value of the positive control.

The dose-response curve was fitted using GraphPad Prism software (version 5, Informer Technologies, Inc., Los Angeles, CA, USA), and the IC₅₀ values for each compounds were calculated by log(inhibitor) vs. response using a variable slope program with the following formula:

Y=Bottom+(Top-Bottom)/(1+10\hat{ }((Log IC₅₀−X)*HillSlope))

Table A1 shows the IC₅₀ values for each of the tested compounds against SRC and PAK1.

TABLE A1

| Compound | SRC | PAK1 |
|---|---|---|
| No. | (μM) | (μM) |
| 1 | +++ | +++ |
| 2 | ND | +++ |
| 3 | +++ | ++ |
| 4 | + | + |
| 5 | + | + |
| 6 | ++ | + |
| 7 | +++ | + |
| 8 | +++ | ND |
| 9 | +++ | ND |
| 10 | + | ND |
| 11 | +++ | ++ |
| 12 | +++ | ++ |
| 13 | +++ | ++ |
| 14 | + | + |
| 15 | ++ | + |
| 16 | ++ | + |
| 17 | +++ | + |
| 18 | +++ | + |
| 19 | +++ | + |
| 20 | +++ | + |
| 21 | + | + |
| 22 | +++ | ++ |
| 23 | + | ++ |
| 24 | + | ++ |
| 25 | + | ++ |
| 26 | +++ | ++ |
| 27 | +++ | + |
| 28 | ++ | + |
| 29 | ++ | + |
| 30 | ND | + |
| 31 | ++ | + |
| 32 | ND | + |
| 33 | ND | ++ |
| 34 | ND | + |
| 35 | ++ | ++ |
| 36 | ++ | + |
| 37 | ND | + |
| 38 | ND | + |
| 39 | + | + |
| 40 | + | + |
| 41 | + | ND |
| 42 | + | + |
| 43 | ++ | + |
| 44 | ++ | + |
| 45 | + | + |
| 46 | + | + |
| 47 | ++ | + |
| 48 | ++ | + |
| 49 | ++ | + |
| 50 | + | + |
| 51 | ++ | ND |
| 52 | +++ | + |
| 53 | + | ND |
| 54 | + | ND |
| 55 | + | ND |
| 56 | + | ND |
| 57 | + | + |
| 58 | ++ | + |
| 60 | ++ | ND |
| 61 | ++ | ND |
| 64 | + | + |
| 66 | ++ | ND |
| 67 | + | ND |
| 68 | ++ | + |
| 69 | +++ | ++ |
| 70 | ++ | + |
| 71 | + | + |
| 72 | + | ++ |
| 73 | + | + |
| 75 | +++ | + |
| 76 | ++ | ND |
| 89 | ND | ++ |
| 92 | ND | ++ |
| 107 | ND | + |

+++: <100 nM;
++: 100-10,000 nM;
+: >10,000 nM;
ND: Not Determined

The effect of the compounds on the activity of HPK1 (MAP4K1) kinase were assessed by Lantha screen assay.
1. 1×kinase buffer was prepared (50 mM HEPES, pH 7.5, 10 mM MgCl2, 4 mM DT
2. Compound and assay plates preparation:
   2-1) The compound was diluted to 100× of the final desired highest inhibitor concentration in reaction by 100% DMSO. For example, if a compound was tested at 5 M, then a solution at 500 M in DMSO was prepared in this step.
   2-2) For each tested compound, the compound was transferred in tubes to one well on a 96-well storage plate and serially diluted by transferring 101 to 701 of 100% DMSO in the next well and so forth for a total of 6 concentrations.
   2-3) 100 μl of 100% DMSO were added to two empty wells to serve as no compound control and no enzyme control in the same 96-well plate. The resulting plate was labeled as the source plate.
   2-4) 40 μl of compound from source plate were transferred to a new 384-well Echo plate as the intermediate plate. 100 nl of each well from the 384-well Echo plate were transferred to a 384-well assay plate in duplicates. For example, A1 of the 384-well Echo plate was transferred to A1 and A2 of the 384-well plate. A2 of the 384-well Echo plate was transferred to A3 and A4 of the 384-well plate, and so on;
3. Kinase reaction: a solution of MAP4K1 in 1× kinase buffer at 2-fold the final concentration of each reagent in the assay was prepared. 5 μl of kinase solution was added to each well of the assay plate, except for control wells without enzyme (add 5 μl of 1× kinase buffer instead). The plate was then shaken, then incubated at room temperature for 10 min. A substrate solution of Fluorescein-PKC and ATP in 1× kinase reaction buffer at 2-fold of the final concentration of each reagent desired in the assay was prepared. 5 μl of substrate solution was added to each well of the assay plate to start reaction. The plate was shaken, then covered, and incubated at room temperature for 90 minutes.

Kinase detection: detection solution of 2-fold of final concentration in Antibody Dilution Buffer was prepared. 10 μl of detection solution was added to each well of the assay plate to stop the reaction. The mixtures were mixed briefly with Centrifuge and incubated at room temperature 60 minutes before reading on a plate reader for fluorescence. Data was collected on Envision with excitation at 340 nm and emission at 520 nm and 495 nm.

Table A2 shows the IC₅₀ values for each of the tested compounds against HPK1.

TABLE A2

| Compound | HPK1 |
|---|---|
| No. | (μM) |
| 100 | ++ |
| 101 | ++ |
| 105 | ++ |
| 107 | ++ |
| 111 | ++ |
| 112 | ++ |
| 113 | +++ |
| 114 | +++ |
| 115 | +++ |
| 116 | ++ |
| 117 | + |
| 118 | ++ |
| 120 | ++ |
| 121 | ++ |
| 122 | + |
| 123 | + |
| 124 | ++ |
| 126 | ++ |
| 127 | ++ |
| 129 | ++ |
| 130 | +++ |
| 131 | +++ |
| 132 | ++ |
| 133 | +++ |
| 135 | ++ |
| 136 | ++ |
| 137 | +++ |
| 138 | +++ |
| 139 | +++ |
| 145 | ++ |
| 146 | + |
| 147 | + |
| 148 | ++ |
| 150 | + |
| 151 | ++ |
| 152 | ++ |
| 153 | ++ |
| 154 | + |
| 155 | + |
| 157 | ++ |
| 159 | ++ |
| 160 | + |
| 161 | +++ |
| 162 | +++ |
| 163 | +++ |
| 165 | +++ |
| 170 | +++ |
| 178 | +++ |
| 179 | ++ |
| 180 | +++ |
| 181 | +++ |
| 182 | ++ |
| 190 | ++ |
| 191 | ++ |
| 192 | + |
| 193 | ++ |
| 194 | ++ |
| 195 | +++ |
| 207 | +++ |
| 208 | +++ |
| 209 | +++ |
| 210 | +++ |
| 211 | +++ |
| 212 | +++ |
| 213 | +++ |
| 214 | +++ |
| 215 | +++ |
| 216 | ++ |
| 219 | ++ |
| 220 | ++ |
| 221 | +++ |
| 222 | +++ |
| 223 | ++ |
| 224 | ++ |
| 225 | +++ |
| 226 | +++ |
| 227 | +++ |
| 228 | +++ |
| 229 | +++ |
| 231 | +++ |
| 232 | +++ |
| 233 | +++ |
| 234 | +++ |
| 235 | ++ |
| 237 | +++ |
| 239 | ++ |
| 240 | +++ |
| 241 | ++ |
| 267 | +++ |
| 269 | +++ |
| 276 | ++ |
| 277 | ++ |
| 278 | + |
| 283 | + |
| 316 | + |
| 317 | ++ |
| 330 | ++ |
| 331 | ++ |
| 340 | + |
| 341 | +++ |
| 342 | ++ |
| 343 | + |
| 344 | + |

+++: <100 nM;
++: 100-10,000 nM;
+: >10,000 nM;
ND: Not Determined

The effect of the compounds on the activity of cKit, RET and AXL kinases were assessed by Mobility shift assay.
1. 1× kinase base buffer and stop buffer for testing kinases were prepared
   1-1) 1× Kinase base buffer (50 mM HEPES, pH 7.5, 0.01% Triton X-100)
   1-2) Stop buffer (100 mM HEPES, pH 7.5, 0.015% Brij-35, 0.2% Coating Reagent #3, 50 mM EDTA)
2. Compound and assay plate preparation: the compound was diluted to 50× of the final desired highest inhibitor concentration in reaction by 100% DMSO. 100 μl of this compound dilution was transferred to a well in a 96-well plate. For example, if the highest desired concentration was 5 μM, then 250 μM of compound DMSO solution was prepared in this step. For all compounds, the compound was serially diluted by transferring 10 μl to 70 μl of 100 DMSO in the next well and so forth for a total of 5 concentrations. 100 μl of 100% DMSO was added to two empty wells for no compound control and no enzyme control in the same 96-well plate. The plate was labeled as source plate. The intermediate plate was prepared by transferring 10 μl of compound from source plate to a new 96-well plate as the intermediate plate, adding 90 μl of 1× kinase buffer to each well of the intermediate plate, mixing the compounds in intermediate plate for 10 min on shaker. 5 μl of each well from the 96-well intermediate plate were transferred to a 384-well plate in duplicates. For example, A1 of the 96-well plate was transferred to A1 and A2 of the 384-well plate. A2 of the 96-well plate was transferred to A3 and A4 of the 384-well plate, and so on.
4. Kinase reaction: The assay plate already contained 5 μl of compound in 10% DMSO. A 2.5× enzyme solution was prepared by adding kinases (cKit, RET, or AXL) and DTT in 1× kinase base buffer. 2.5× enzyme solution was transferred to the assay plate (by adding 10 μl of 2.5× enzyme solution to each well of the 384-well assay plate). The plate was incubated at room temperature for 10 min. 2.5× peptide solution was prepared by adding FAM-labeled peptide, ATP, MgCl2 in the 1× kinase base buffer. 2.5× peptide solution was added to the assay plate by adding 10 μl of 2.5× peptide solution to each well of the 384-well assay plate. The plate was incubated at 28° C. for the specified period of time. 25 μl of stop buffer was added to stop reaction. Data was collected on Caliper.

Table A3 shows the IC₅₀ values for each of the tested compounds against c-Kit.

TABLE A3

| Compound | c-Kit |
|---|---|
| No. | (μM) |
| 92 | ++ |
| 102 | +++ |
| 103 | +++ |
| 107 | +++ |
| 115 | +++ |
| 136 | +++ |
| 140 | ++ |
| 178 | ++ |
| 188 | +++ |
| 195 | +++ |
| 196 | +++ |
| 229 | +++ |
| 285 | + |
| 287 | ++ |
| 289 | + |
| 312 | + |
| 313 | ++ |
| 315 | + |
| 316 | ++ |
| 317 | +++ |
| 326 | +++ |
| 327 | +++ |
| 328 | +++ |
| 329 | +++ |
| 330 | +++ |
| 331 | +++ |
| 332 | +++ |
| 333 | +++ |
| 334 | +++ |
| 335 | +++ |
| 336 | ++ |
| 337 | +++ |
| 338 | +++ |

+++: <100 nM;
++: 100-10,000 nM;
+: >10,000 nM;
ND: Not Determined

Table A4 shows the IC₅₀ values for each of the tested compounds against RET.

TABLE A4

| Compound | RET |
|---|---|
| No. | (μM) |
| 107 | +++ |
| 111 | +++ |
| 120 | ++ |
| 121 | ++ |
| 122 | + |
| 129 | ++ |
| 130 | +++ |
| 135 | +++ |
| 136 | +++ |
| 208 | +++ |
| 340 | ++ |
| 341 | ++ |

+++: <100 nM;
++: 100-10,000 nM;
+: >10,000 nM;
ND: Not Determined

Table A5 shows the IC₅₀ values for each of the tested compounds against Axl.

TABLE A5

| Compound | Axl |
|---|---|
| No. | (μM) |
| 193 | + |
| 266 | + |
| 270 | + |
| 273 | ++ |
| 275 | ++ |
| 276 | + |
| 285 | + |
| 286 | + |
| 287 | +++ |
| 289 | + |
| 292 | + |
| 293 | + |
| 296 | + |
| 297 | + |
| 298 | + |
| 299 | + |
| 302 | + |
| 303 | + |
| 330 | + |
| 333 | + |
| 335 | + |
| 336 | + |
| 340 | + |
| 341 | + |
| 345 | ++ |

+++: <100 nM;
++: 100-10,000 nM;
+: >10,000 nM;
ND: Not Determined

### Biological Example B2

MDCK-MDR1 Permeability Assay

MDCK-MDR1 cells originate from transfection of Madin Darby canine kidney (MDCK) cells with the MDR1 gene, the gene encoding for the efflux protein, P-glycoprotein. This cell line is ideal for identifying substrates of P-gp, with or without an inhibitor. The cells are seeded on a Multiscreen™ plate to form a confluent monolayer over 4 days prior to the experiment. On day 4, the test compound (1-30 μM concentration) is added to the apical side of the membrane and the transport of the compound across the monolayer is monitored over a 120 minutes time period. To study drug efflux, it is also necessary to investigate transport of the compound from the basolateral compartment to the apical compartment and calculate an efflux ratio.

The permeability coefficient (Pₐₚₚ) is calculated from the following equation:

Pₐₚₚ=[(dQ/dt)/C₀xA]

where dQ/dt is the rate of permeation of the drug across the cells, C₀ is the donor compartment concentration at time zero and A is the area of the cell monolayer.

An efflux ratio is calculated from the mean apical to basolateral (A-B) Pₐₚₚ data and basolateral to apical (B-A) Pₐₚₚ data.

Efflux Ratio=Pₐₚₚ(B−A)/Pₐₚₚ(A−B)

Table B summarizes the permeability of Compound 1 in a MDCK-MDR1 Assay.

TABLE B

| Cmpd No. | Papp (a to b) | Papp (b to a) | Efflux Ratio |
|---|---|---|---|
| 1 | 1.5 | 12.6 | 8.4 |
| 52 | 1.0 | 2.4 | 2.3 |
| 69 | 3.6 | 30.3 | 8.3 |
| 76 | 2.7 | 35.2 | 13.1 |
| 105 | 5.6 | 61.2 | 10.9 |
| 106 | 44.4 | 50.9 | 1.1 |
| 107 | 58.3 | 59.5 | 1.0 |
| 115 | 22.8 | 36.9 | 1.6 |
| 135 | 24.4 | 29.4 | 1.2 |
| 136 | 15.6 | 29.1 | 1.9 |
| 139 | 19.1 | 10.0 | 0.5 |
| 195 | 5.5 | 28.4 | 5.1 |
| 207 | 1.2 | 7.6 | 6.5 |
| 208 | 0.8 | 3.3 | 4.0 |
| 214 | 0.7 | 0.7 | 1.1 |
| 221 | 0.4 | 1.5 | 4.1 |
| 222 | 0.4 | 0.6 | 1.5 |
| 225 | 0.1 | 0.1 | 0.6 |
| 226 | <1 | <1 | NA |
| 229 | 0.4 | 24.2 | 53.7 |
| 330 | 0.3 | 6.7 | 26.6 |
| 331 | 8.9 | 34.9 | 3.9 |

### Biological Example B3

Mouse Pharmacokinetics Study

The pharmacokinetic properties of Compound 1 was studied in CD-1 mice via intravenous and oral administration by using a standard protocol. The test articles were formulated in 2000 Hydroxypropyl-beta-cyclodextrin, either as a clear solution or fine suspension. Table D shows the pharmacokinetic characterization by intravenous injection of Compound 1 in mice.

TABLE D

| Cmpd | IV Dose | t_{1/2} | Clₚ | V_{d} |
|---|---|---|---|---|
| No. | (mg/kg) | (h) | (ml/min · kg) | (L/kg) |
| 1 | 5 | 11.7 ± 2.2 | 64.6 ± 6.8 | 65.1 ± 13.4 |
| 106 | 5 | 1.42 ± 0.20 | 27.7 ± 7.7 | 3.36 ± 0.87 |
| 107 | 5 | 3.14 ± 0.05 | 15.2 ± 3.3 | 4.13 ± 0.85 |
| 135 | 5 | 1.82 ± 0.21 | 47.6 ± 14.3 | 7.36 ± 1.67 |
| 136 | 5 | 3.78 ± 0.16 | 19.1 ± 1.8 | 6.25 ± 0.76 |
| 163 | 5 | 1.57 ± 0.20 | 48.8 ± 5.8 | 6.61 ± 1.04 |
| 178 | 5 | 2.76 ± 0.22 | 17.0 ± 0.30 | 4.06 ± 0.37 |
| 181 | 5 | 2.01 ± 0.82 | 28.5 ± 3.8 | 4.78 ± 1.21 |
| 195 | 5 | 3.78 ± 0.53 | 21.4 ± 2.9 | 7.04 ± 1.56 |
| 202 | 5 | 3.99 ± 0.72 | 27.8 ± 1.0 | 9.64 ± 1.99 |
| 207 | 5 | 3.62 ± 0.19 | 31.3 ± 5.5 | 9.81 ± 2 |
| 208 | 5 | 4.66 ± 0.35 | 20.7 ± 1.3 | 8.36 ± 0.88 |
| 331 | 5 | 2.45 ± 0.62 | 31.9 ± 6.3 | 6.55 ± 0.33 |

Table E shows the plasma exposure by oral administration of Compound 1 in mice.

TABLE E

| Cmpd | PO Dose | Cₘₐₓ | Tₘₐₓ | AUC | F |
|---|---|---|---|---|---|
| No. | (mg/kg) | (ng/ml) | (h) | (ng/mL · h) | (%) |
| 1 | 50 | 504 ± 190 | 1.17 ± 0.76 | 3861 ± 952 | 34.8 ± 8.6 |
| 106 | 25 | 1933 ± 263 | 0.417 ± 0.144 | 10897 ± 5367 | 69.2 ± 34.1 |
| 107 | 25 | 3540 ± 326 | 1.00 ± 0.00 | 26034 ± 6442 | 92.7 ± 23.2 |
| 135 | 25 | 1377 ± 195 | 0.75 ± 0.43 | 4682 ± 2157 | 50.8 ± 23.4 |
| 136 | 25 | 2170 ± 540 | 1.50 ± 0.87 | 21485 ± 678 | 105 ± 1 |
| 139 | 25 | 1790 ± 286 | 1.67 ± 0.58 | 18368 ± 1233 | NA |
| 163 | 25 | 1100 ± 497 | 1.33 ± 0.58 | 6318 ± 4576 | 74.6 ± 54 |
| 178 | 25 | 2563 ± 505 | 2.00 ± 0.00 | 16305 ± 3488 | 66.7 ± 14.3 |
| 181 | 25 | 1757 ± 380 | 2.00 ± 0.00 | 8607 ± 903 | 58.1 ± 6.1 |
| 195 | 25 | 882 ± 111 | 3.67 ± 3.79 | 10893 ± 1233 | 63.4 ± 8 |
| 196 | 25 | 1058 ± 108 | 1.33 ± 0.58 | 11331 ± 3010 | NA |
| 199 | 25 | 245 ± 37 | 2.00 ± 0.00 | 1726 ± 619 | NA |
| 202 | 25 | 839 ± 59 | 1.67 ± 0.58 | 8159 ± 1270 | 56 ± 9 |
| 207 | 25 | 1079 ± 127 | 2.00 ± 0.00 | 9644 ± 987 | 71.7 ± 7.8 |
| 208 | 25 | 1253 ± 337 | 2.00 ± 0.00 | 15433 ± 2154 | 78.3 ± 10.9 |
| 231 | 25 | 1189 ± 302 | 1.67 ± 0.58 | 16194 ± 2103 | NA |
| 233 | 25 | 893 ± 258 | 6.67 ± 2.31 | 12013 ± 2751 | NA |
| 234 | 25 | 1630 ± 151 | 2.00 ± 0.00 | 20466 ± 2187 | NA |
| 242 | 25 | 3063 ± 253 | 1.67 ± 0.58 | 36854 ± 4056 | NA |
| 256 | 25 | 1180 ± 193 | 1.33 ± 0.58 | 11726 ± 1432 | NA |
| 331 | 25 | 570 ± 234 | 1.33 ± 0.58 | 5310 ± 2207 | 39.6 ± 16.4 |

### Biological Example B4

In Vivo Pharmacodynamic Study

The activity of the compounds of formula (I), in vivo, can be determined by the amount of inhibition of tumor growth by a test compound relative to a control. The tumor growth inhibitory effects of various compounds are measured according to the method of Corbett T. H., et al., “Tumor Induction Relationships in Development of Transplantable Cancers of the Colon in Mice for Chemotherapy Assays, with a Note on Carcinogen Structure”, Cancer Res., 35, 2434-2439 (1975) and Corbett T. H., et al., “A Mouse Colon-tumor Model for Experimental Therapy”, Cancer Chemother. Rep. (Part 2)”, 5, 169-186 (1975), with slight modifications. Tumors are induced in the left flank by subcutaneous injection of 1-5 million log phase cultured tumor cells (human A375 melanoma or HT-29 colorectal cancer cells) suspended in 0.1 ml RPMI 1640 medium. After sufficient time has elapsed for the tumors to become palpable (100-150 mm³ in size/5-6 mm in diameter) the test animals (BALB/c nude female mice) are treated with test compound (formulated at a concentration of 10 to 15 mg/ml in 20% hydroxypropyl-beta-cyclodextrine) by oral route of administration once or twice daily. In order to determine an anti-tumor effect, the tumor is measured in millimeters with a Vernier caliper across two diameters and the tumor size (mm3) is calculated using the formula: Tumor size (mm³)=(length×width²)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula: Inhibition (%)=(TuW_{control}−TuWₜₑₛₜ)/TuW_{control}×100%. The flank site of tumor implantation provides reproducible dose/response effects for a variety of chemotherapeutic agents, and the method of measurement (tumor diameter) is a reliable method for assessing tumor growth rates.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

1. A compound of Formula (I): 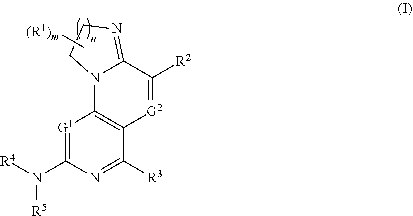
or a pharmaceutically acceptable salt, solvate or isotopic derivative thereof, wherein:
G¹ is N or CR^{a};
G² is N or CR^{b};
n is 1 or 2;
m is 0, 1, 2 or 3;
R^{a} and R^{b} are each independently selected from the group consisting of hydrogen, halogen, cyano, nitro, hydroxy, optionally substituted C₁-C₆ alkyl, optionally substituted aryl, optionally substituted heteroaryl, optionally substituted C₁-C₆ alkoxy, optionally substituted C₁-C₆ alkylamino, and optionally substituted aryloxy;
each R¹ is independently selected from the group consisting of halogen, optionally substituted C₁-C₆ alkyl, and optionally substituted C₁-C₆ alkoxy;
or two R¹ groups with the carbon atom they connect to form a 4- to 7-membered carbocyclic or heterocyclic ring, which is optionally substituted by one or more R^{a} groups;
R² is selected from the group consisting of hydrogen, optionally substituted C₁-C₆ alkyl, optionally substituted C₁-C₆ alkyl-O, optionally substituted C₁-C₆ alkyl-S, optionally substituted C₁-C₆ alkyl-SO₂, optionally substituted C₁-C₆ alkyl-NR^{a}, optionally substituted C₂-C₆ alkenyl, optionally substituted C₂-C₆ alkynyl, optionally substituted aryl, optionally substituted aryl-O, optionally substituted aryl-S, optionally substituted aryl-SO₂, optionally substituted aryl-NR^{a}, optionally substituted heteroaryl, optionally substituted heteroaryl-O, optionally substituted heteroaryl-S, optionally substituted heteroaryl-SO₂, optionally substituted heteroaryl-NR^{a}, optionally substituted cycloalkyl, optionally substituted cycloalkyl-O, optionally substituted cycloalkyl-S, optionally substituted cycloalkyl-SO₂, optionally substituted cycloalkylNR^{a}, optionally substituted heterocyclyl, optionally substituted heterocyclyl-O, optionally substituted heterocyclyl-S, optionally substituted heterocyclyl-SO₂, and optionally substituted heterocyclyl-NR^{a};
R³ is selected from the group consisting of hydrogen, halogen, optionally substituted C₁-C₆ alkyl, and optionally substituted C₁-C₆ alkoxy;
R⁴ is selected from the group consisting of:
(i) hydrogen;
(ii) C₁-C₆ alkyl optionally substituted with one or more groups selected from the group consisting of halogen, C₁-C₆ alkoxy, hydroxyl, heteroaryl, and optionally substituted heterocyclyl;
(iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
(iv) aryl optionally substituted with one or more groups selected from the group consisting of halogen, hydroxyl, —C(O)O(C₁-C₆ alkyl), acryloylamino, optionally substituted C₁-C₆ alkyl, optionally substituted C₁-C₆ alkoxy, optionally substituted C₁-C₆ thioalkoxy, optionally substituted C₁-C₆ alkyl-NR^{a}, and optionally substituted heterocyclyl;
(v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, optionally substituted C₁-C₆ alkyl-NR^{a}, and optionally substituted heterocyclyl;
(vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
(vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl; and
provided that when R² is 2,6-dichloro-3,5-dimethoxyphenyl, then:
R⁴ is selected from the group consisting of:
(i) hydrogen;
(ii) C₁-C₆ alkyl optionally substituted with one or more groups selected from the group consisting of halogen, C₁-C₆ alkoxy, hydroxyl, heteroaryl, and optionally substituted heterocyclyl;
(iii) heterocyclyl optionally substituted with one or more groups selected from the group consisting of halogen, hydroxyl, acryloyl, optionally substituted C₁-C₆ alkyl, optionally substituted C₁-C₆ alkoxy, optionally substituted C₁-C₆ thioalkoxy, and optionally substituted heterocyclyl;
(iv) aryl optionally substituted with one or more groups selected from the group consisting of halogen, hydroxyl, —C(O)O(C₁-C₆ alkyl), optionally substituted C₁-C₆ alkyl, optionally substituted C₁-C₆ alkoxy, optionally substituted C₁-C₆ thioalkoxy, and optionally substituted heterocyclyl;
(v) heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heteroaryl, and optionally substituted heterocyclyl;
(vi) carbonyl substituted with C₁-C₆ alkyl, C₃-C₆ cycloalkyl, C₁-C₆ alkoxy, —NR^{a}(C₁-C₆ alkoxy), and optionally substituted heteroaryl; and
(vii) —SO₂(C₁-C₆ alkyl); and
R⁵ is hydrogen, C₁-C₆ alkyl, or heterocyclyl;
or R⁴ and R⁵ are taken together with the nitrogen to which they are attached to form an optionally substituted heterocyclyl or an optionally substituted heteroaryl.

2. The compound of claim 1, or a pharmaceutically acceptable salt, solvate or isotopic derivative thereof, wherein G¹ is N.

3. The compound of claim 1, or a pharmaceutically acceptable salt, solvate or isotopic derivative thereof, wherein G¹ is CR.

4. The compound of claim 3, or a pharmaceutically acceptable salt, solvate or isotopic derivative thereof, wherein R^{a} is hydrogen.

5. The compound of any one of claims 1-4, or a pharmaceutically acceptable salt, solvate or isotopic derivative thereof, wherein G² is N.

6. The compound of any one of claims 1-4, or a pharmaceutically acceptable salt, solvate or isotopic derivative thereof, wherein G² is CR^{b}.

7. The compound of claim 6, or a pharmaceutically acceptable salt, solvate or isotopic derivative thereof, wherein R^{b} is hydrogen.

8. The compound of claim 1, wherein the compound is of Formula (I-1a): 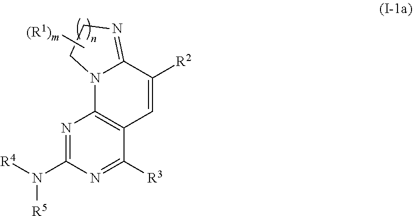
or a pharmaceutically acceptable salt, solvate or isotopic derivative thereof, wherein R¹, R², R³, R⁴, R⁵, m, and n are as defined for Formula (I).

9. The compound of any one of claims 1-8, or a pharmaceutically acceptable salt, solvate or isotopic derivative thereof, wherein n is 1.

10. The compound of any one of claims 1-8, or a pharmaceutically acceptable salt, solvate or isotopic derivative thereof, wherein n is 2.

11. The compound of claim 1, wherein the compound is of Formula (I-2a) or (I-2b): 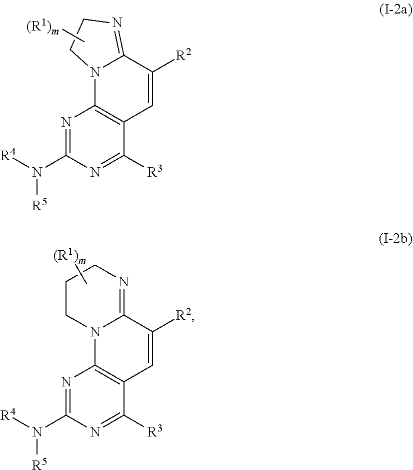
or a pharmaceutically acceptable salt, solvate or isotopic derivative thereof, wherein R¹, R², R³, R⁴, R⁵, and m are as defined for Formula (I).

12. The compound of any one of claims 1-11, or a pharmaceutically acceptable salt, solvate or isotopic derivative thereof, wherein m is 0.

13. The compound of any one of claims 1-11, or a pharmaceutically acceptable salt, solvate or isotopic derivative thereof, wherein each m is 1, and R¹ is independently selected from the group consisting of halogen, optionally substituted C₁-C₆ alkyl, and optionally substituted C₁-C₆ alkoxy.

14. The compound of any one of claims 1-11, or a pharmaceutically acceptable salt, solvate or isotopic derivative thereof, wherein each m is 2, or 3, and each R¹ is independently selected from the group consisting of halogen, optionally substituted C₁-C₆ alkyl, and optionally substituted C₁-C₆ alkoxy; or two R¹ groups with the carbon atom they connect to form a 4- to 7-membered carbocyclic or heterocyclic ring, which is optionally substituted by one or more R^{a} groups.

15. The compound of claim 1, wherein the compound is of Formula (I-3a) or (I-3b): 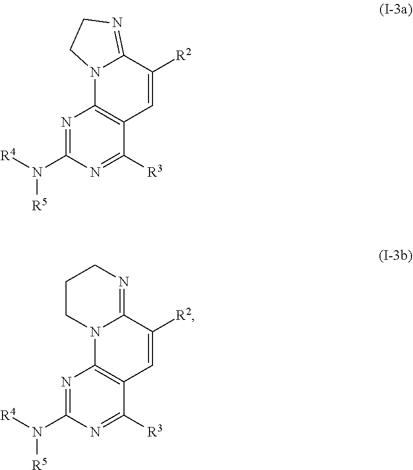
or a pharmaceutically acceptable salt, solvate or isotopic derivative thereof, wherein R², R³, R⁴, and R⁵ are as defined for Formula (I).

16. The compound of any one of claims 1-15, or a pharmaceutically acceptable salt, solvate or isotopic derivative thereof, wherein R² is hydrogen.

17. The compound of any one of claims 1-15, or a pharmaceutically acceptable salt, solvate or isotopic derivative thereof, wherein R² is optionally substituted C₁-C₆ alkyl, optionally substituted C₂-C₆ alkenyl, or optionally substituted C₂-C₆ alkynyl.

18. The compound of any one of claims 1-15, or a pharmaceutically acceptable salt, solvate or isotopic derivative thereof, wherein R² is optionally substituted aryl.

20. The compound of any one of claims 1-15, or a pharmaceutically acceptable salt, solvate or isotopic derivative thereof, wherein R² is optionally substituted heteroaryl.

21. The compound of any one of claims 1-14, or a pharmaceutically acceptable salt, solvate or isotopic derivative thereof, wherein R² is optionally substituted heterocyclyl.

22. The compound of claim 22, or a pharmaceutically acceptable salt, solvate or isotopic derivative thereof, wherein R² is selected from the group consisting of H,

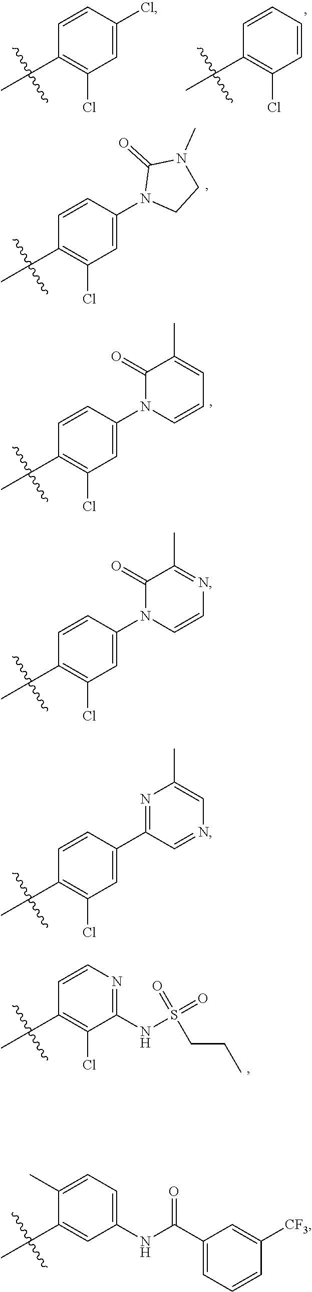

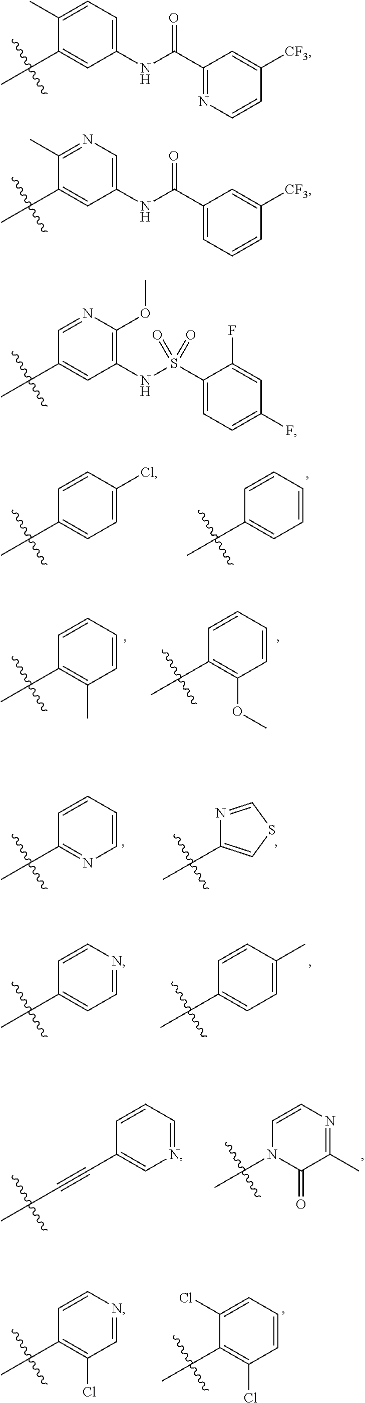

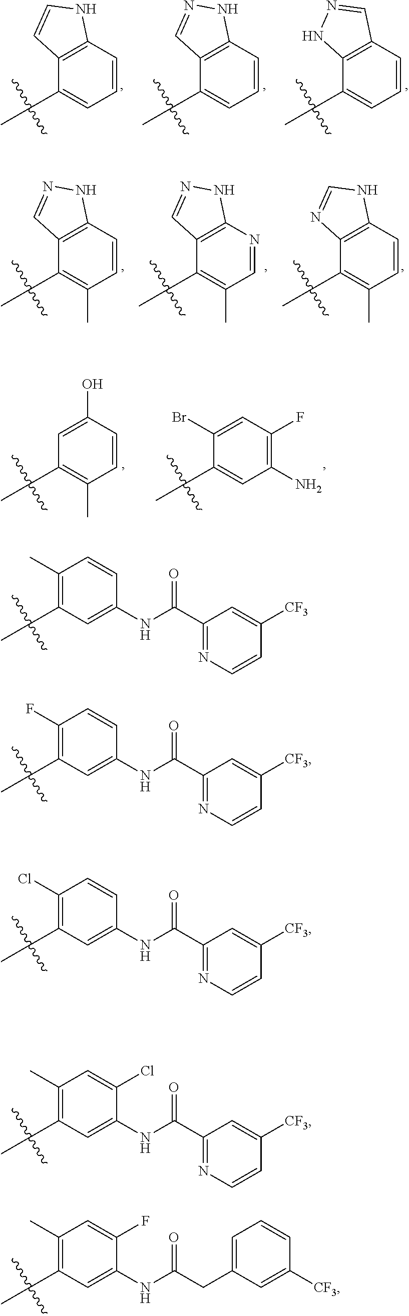

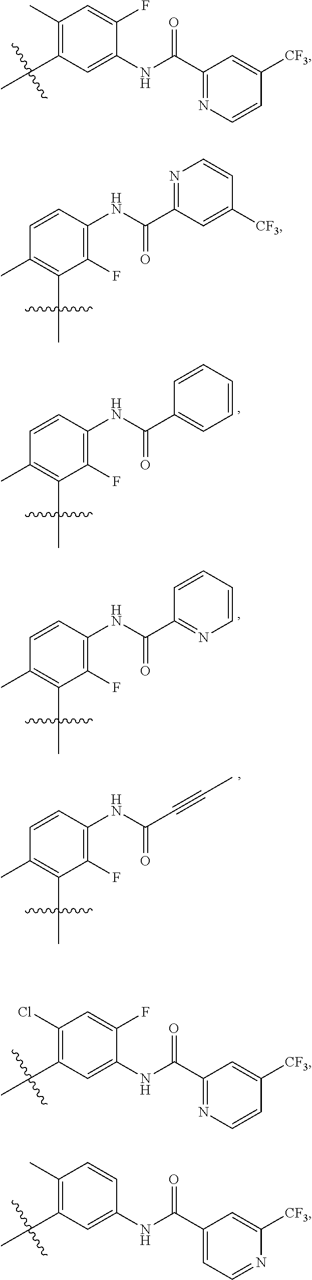

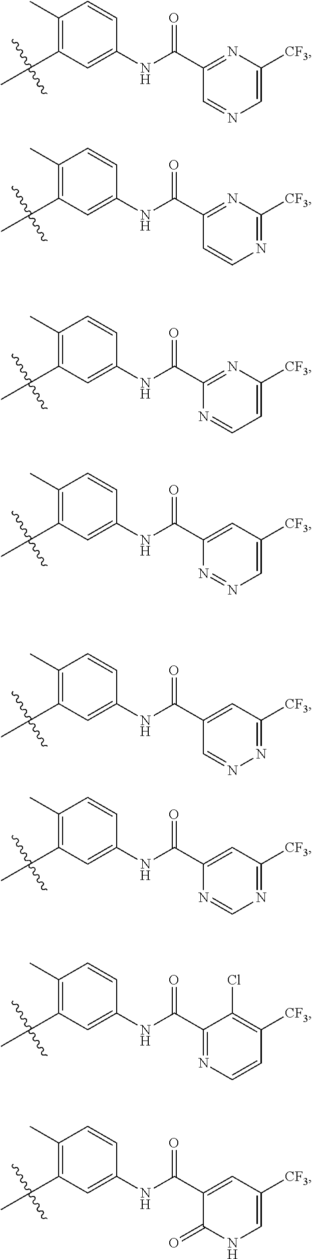

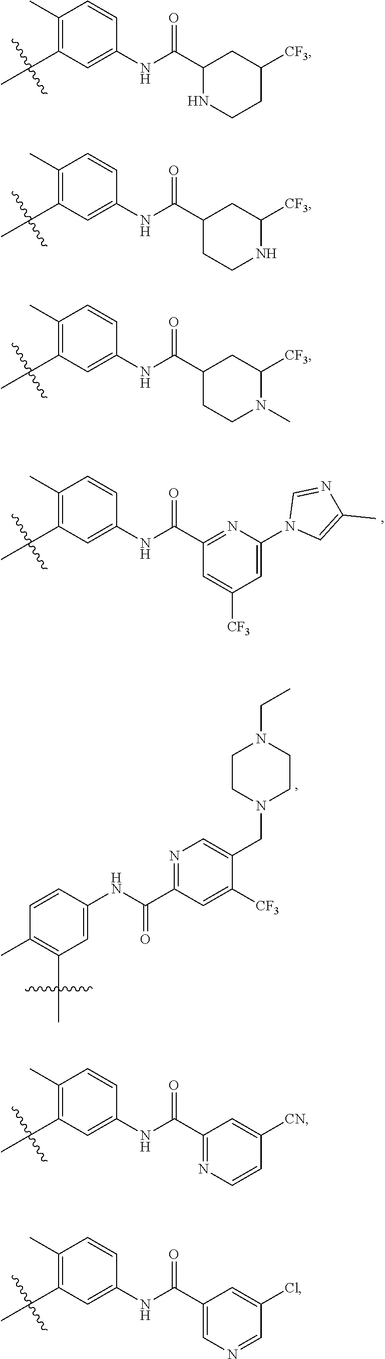

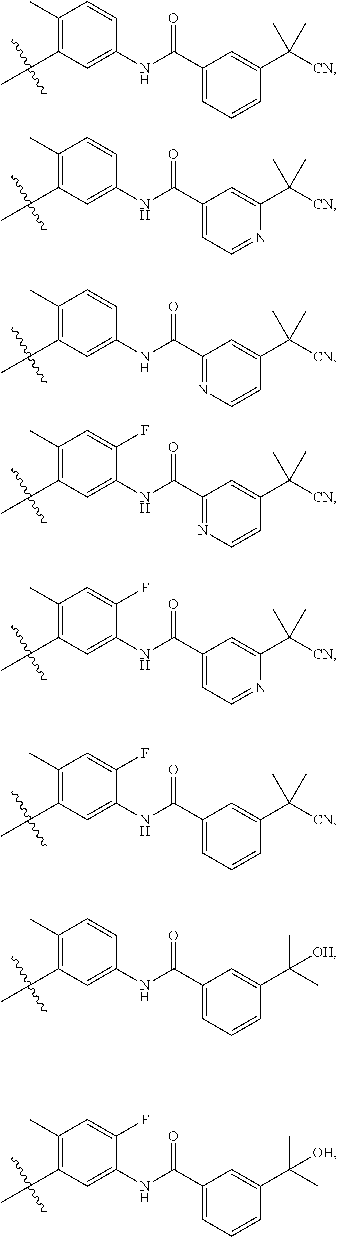

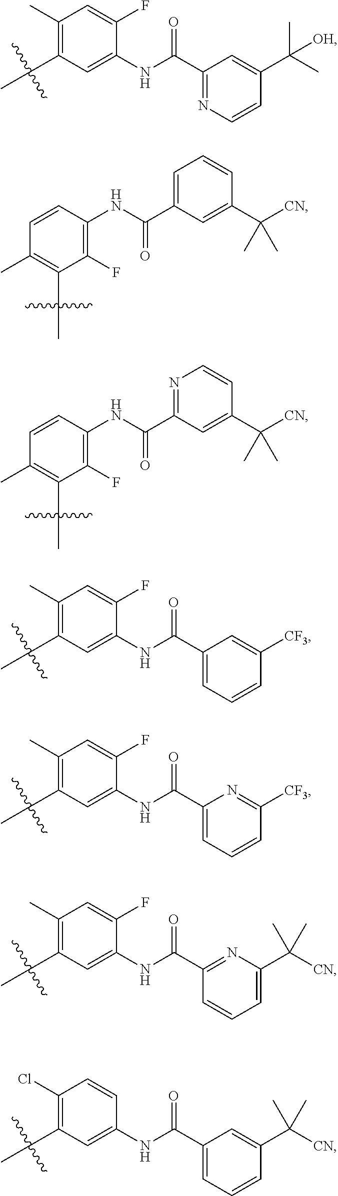

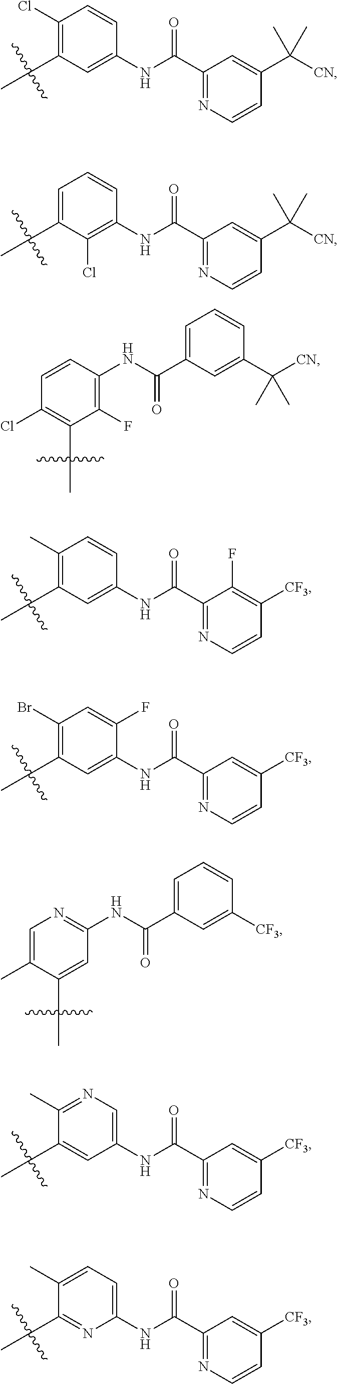

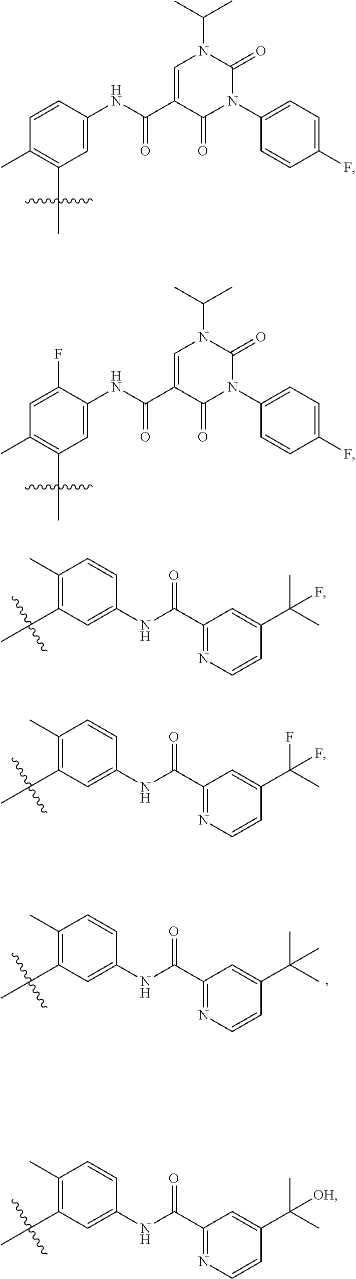

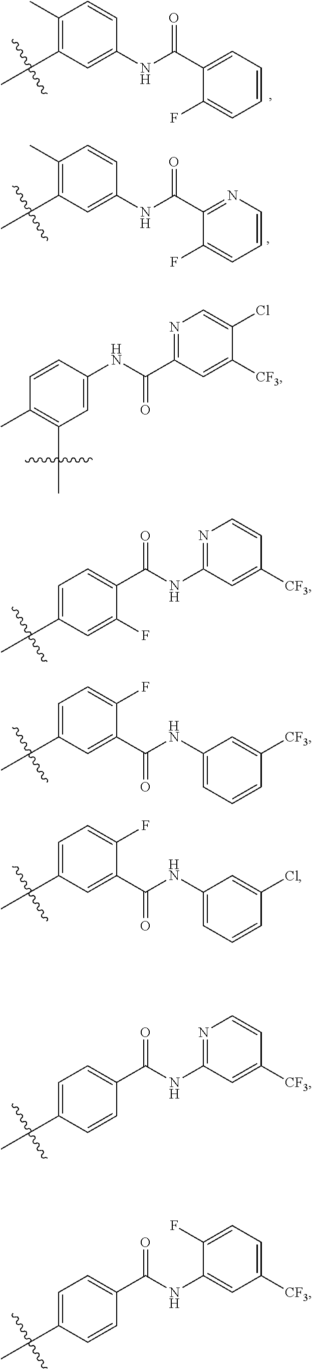

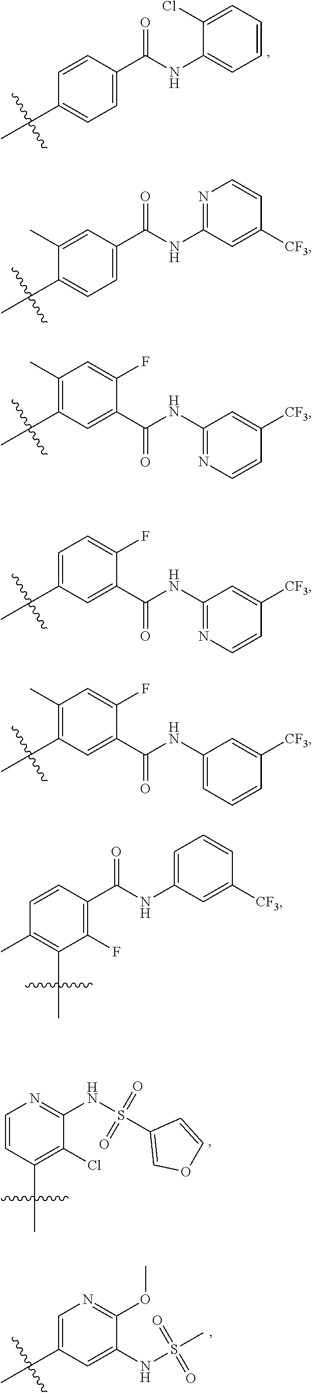

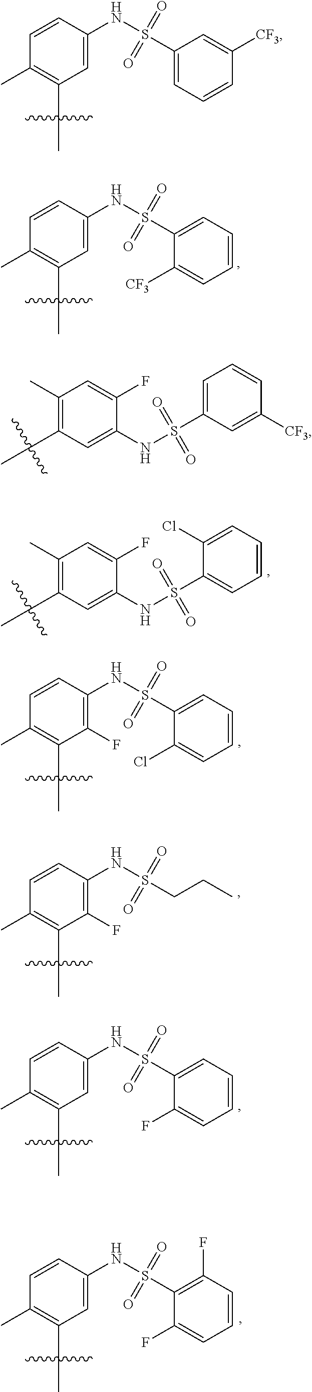

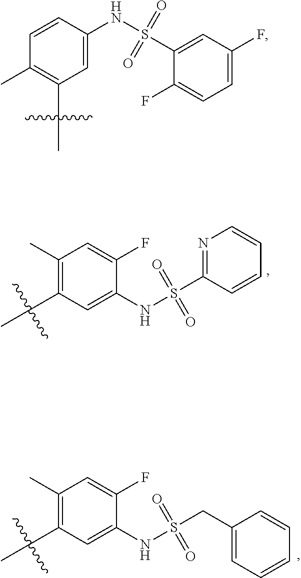 [SMILES: *c1cc(NS(=O)(=O)c2cc(F)ccc2F)ccc1C ; *c1cc(NS(=O)(=O)c2ccccn2)c(F)cc1C ; *c1cc(NS(=O)(=O)Cc2ccccc2)c(F)cc1C]

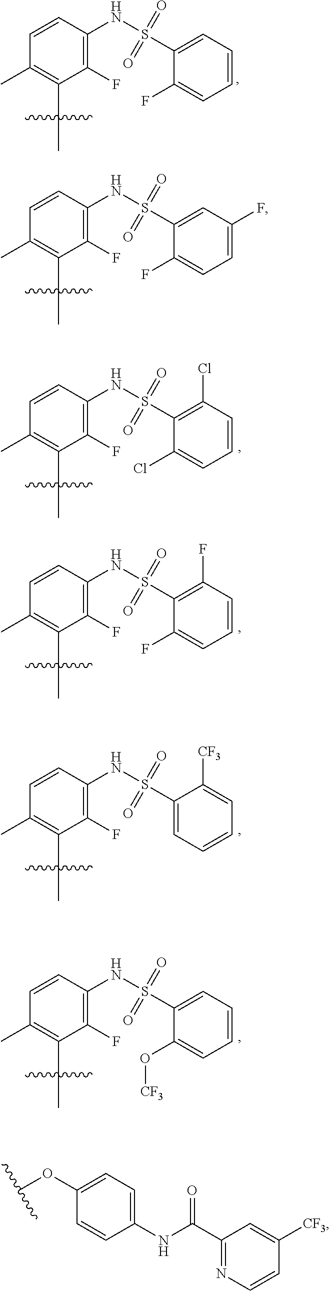

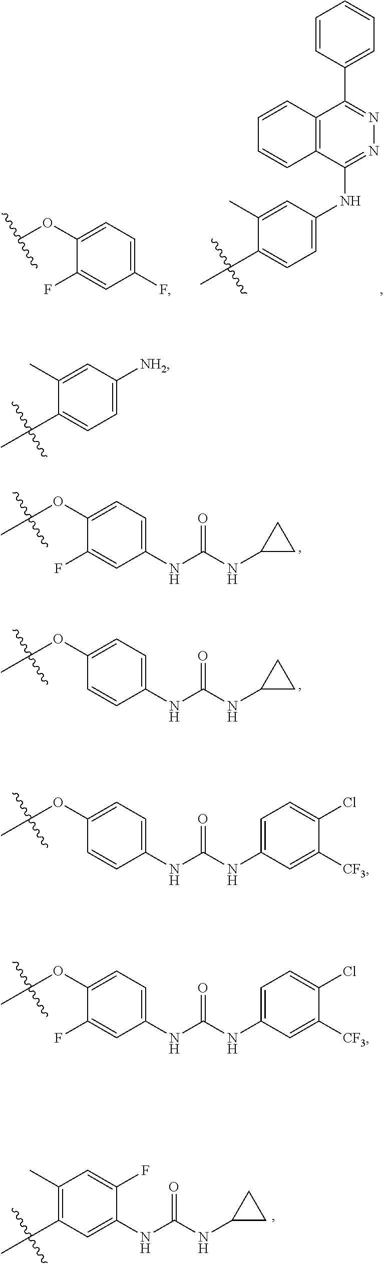

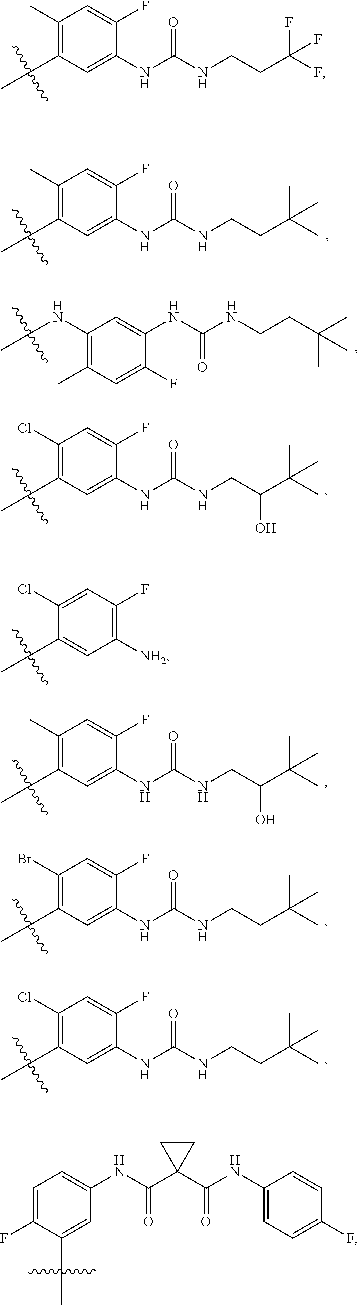

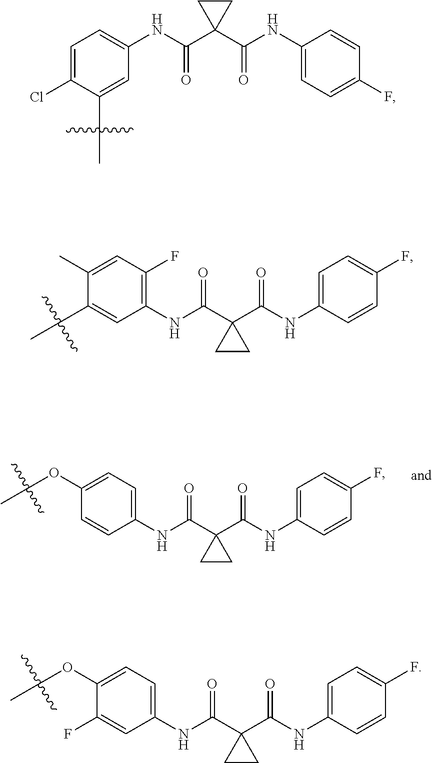 [SMILES: *c1cc(NC(=O)C2(C(=O)Nc3ccc(F)cc3)CC2)ccc1Cl ; *c1cc(NC(=O)C2(C(=O)Nc3ccc(F)cc3)CC2)c(F)cc1C ; *Oc1ccc(NC(=O)C2(C(=O)Nc3ccc(F)cc3)CC2)cc1 ; *Oc1ccc(NC(=O)C2(C(=O)Nc3ccc(F)cc3)CC2)cc1F]

23. The compound of any one of claims 1-22, or a pharmaceutically acceptable salt, solvate or isotopic derivative thereof, wherein R³ is hydrogen.

24. The compound of any one of claims 1-22, or a pharmaceutically acceptable salt, solvate or isotopic derivative thereof, wherein R⁴ is C₁-C₆ alkyl optionally substituted with optionally substituted heterocyclyl.

25. The compound of any one of claims 1-22, or a pharmaceutically acceptable salt, solvate or isotopic derivative thereof, wherein R⁴ is heterocyclyl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl.

26. The compound of any one of claims 1-22, or a pharmaceutically acceptable salt, solvate or isotopic derivative thereof, wherein R⁴ is aryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, optionally substituted heterocyclyl.

27. The compound of any one of claims 1-22, or a pharmaceutically acceptable salt, solvate or isotopic derivative thereof, wherein R⁴ is heteroaryl optionally substituted with one or more groups selected from the group consisting of halogen, hydroxyl, optionally substituted C₁-C₆ alkyl, optionally substituted C₁-C₆ alkoxy, optionally substituted C₁-C₆ thioalkoxy, and optionally substituted heterocyclyl.

28. The compound of any one of claims 1-22, or a pharmaceutically acceptable salt, solvate or isotopic derivative thereof, wherein R⁴ is selected from the group consisting of hydrogen, methyl, ethyl,

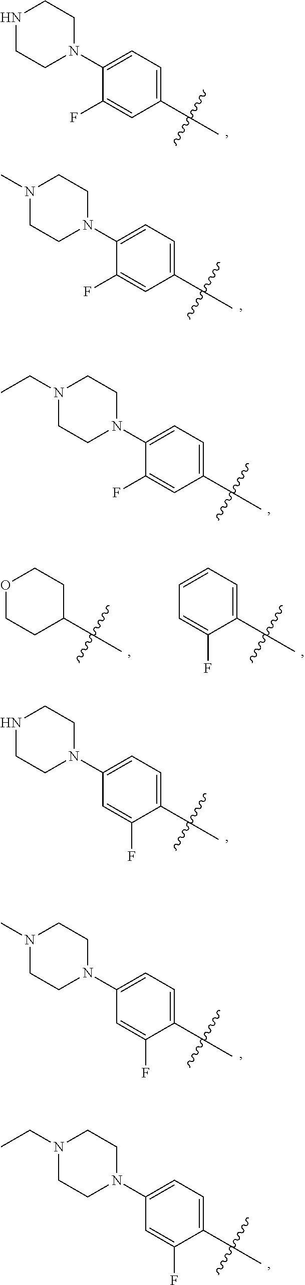

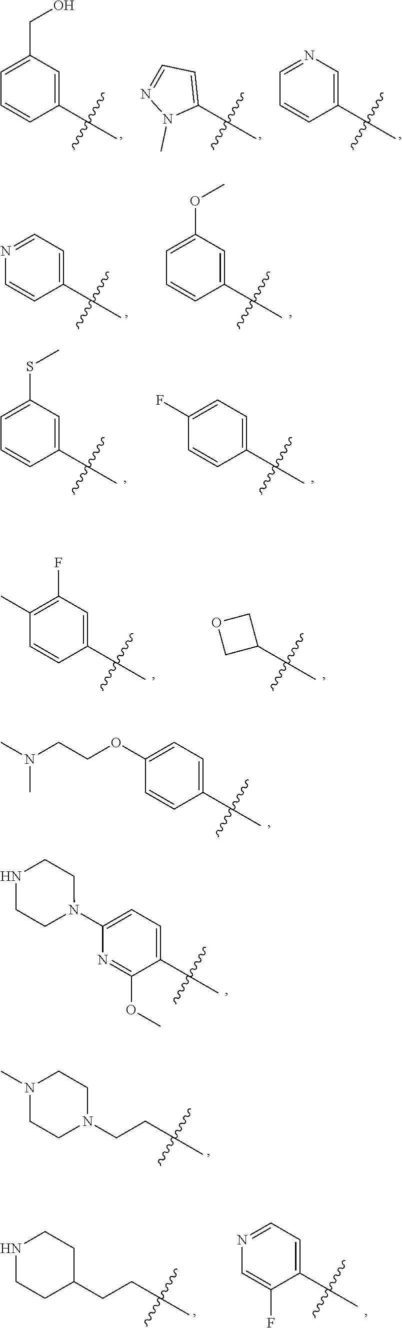

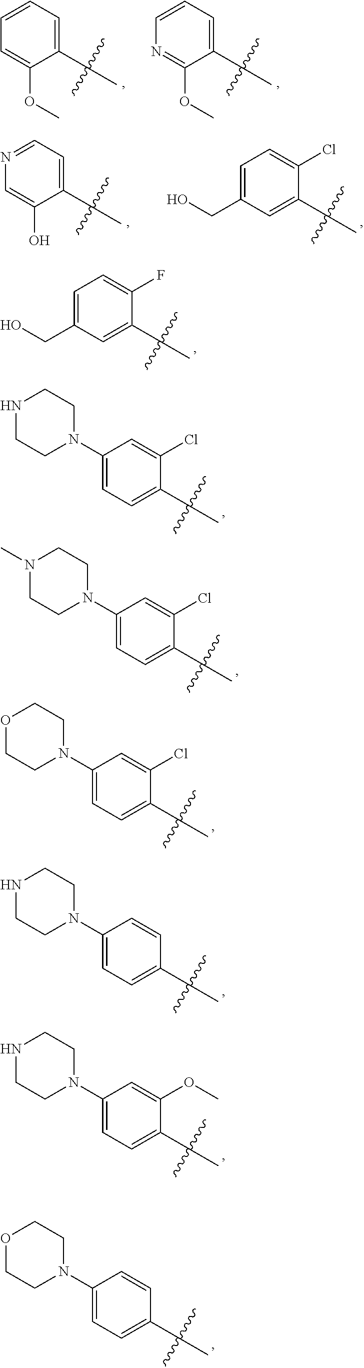

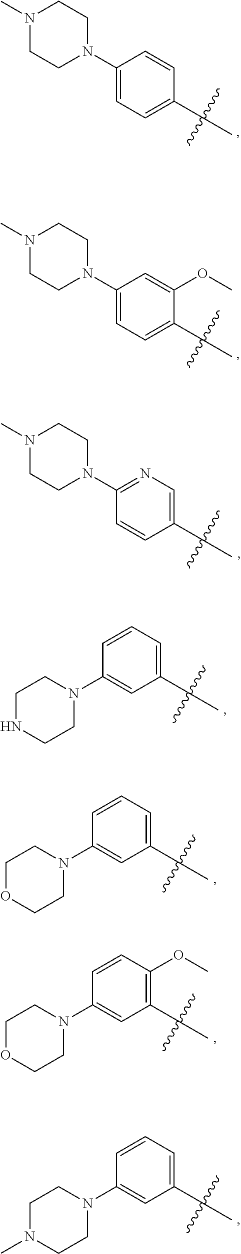

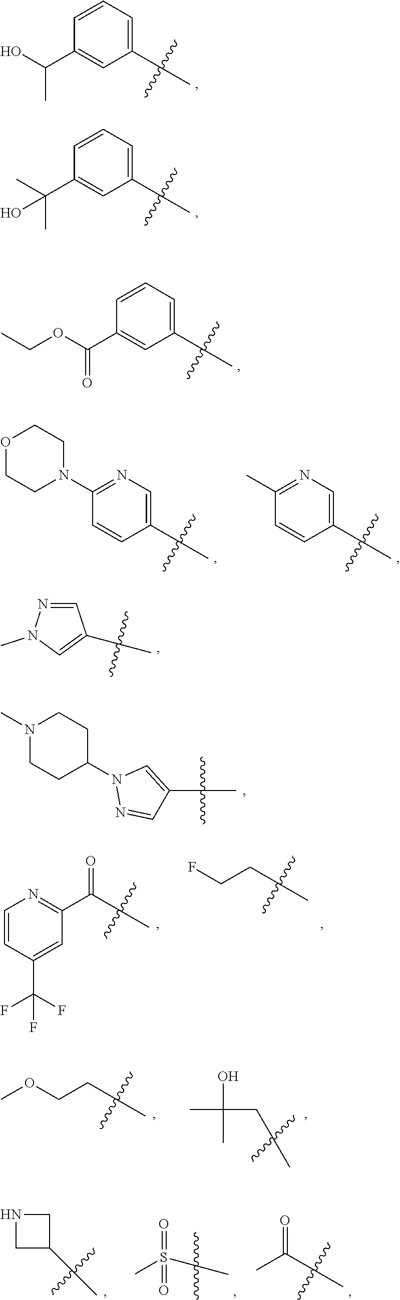

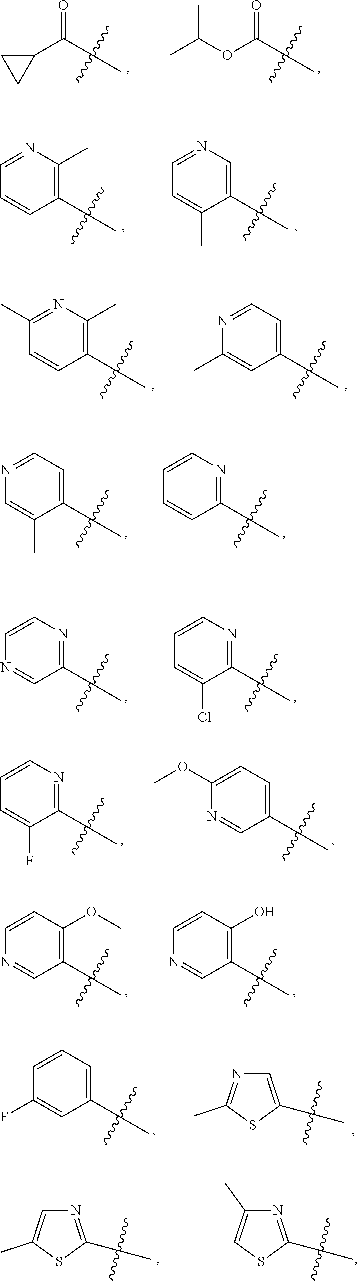

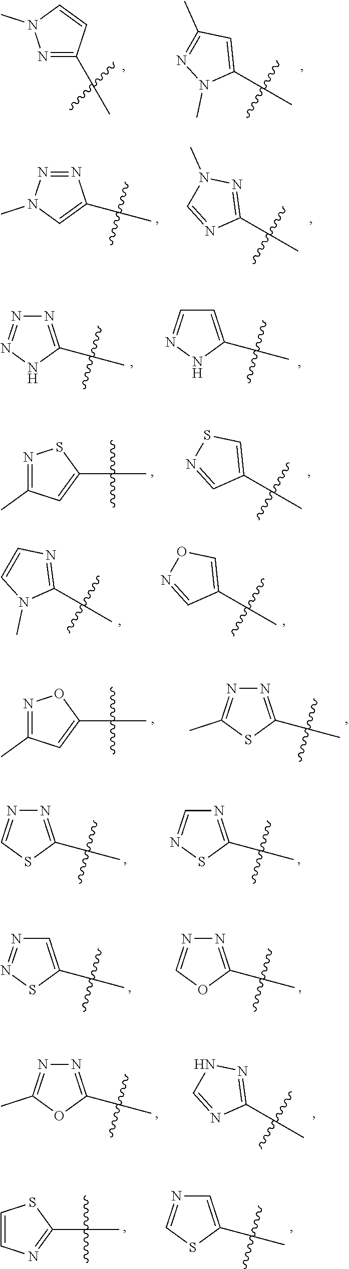

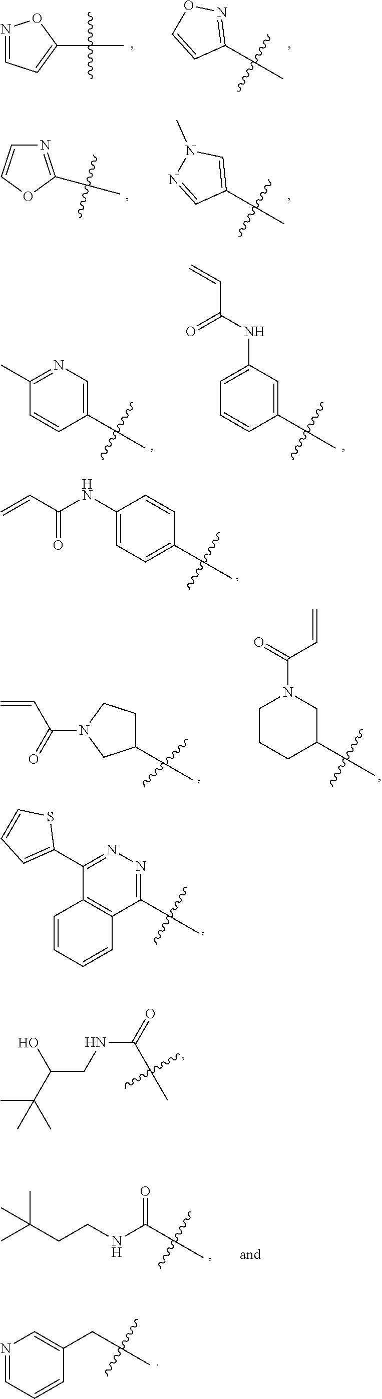

29. The compound of any one of claims 1-28, or a pharmaceutically acceptable salt, solvate or isotopic derivative thereof, wherein R⁵ is hydrogen.

30. The compound of any one of claims 1-22, or a pharmaceutically acceptable salt, solvate or isotopic derivative thereof, wherein R⁴ and R⁵ are taken together with the nitrogen to which they are attached to form an optionally substituted heterocyclyl.

31. A compound selected from a compound of Table 1:

| Compound | | |
|---|---|---|
| No. | Structure | Chemical Name |
| 1 | 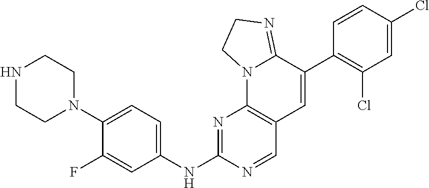 [SMILES: Fc1cc(Nc2ncc3c(n2)N2CCN=C2C(c2ccc(Cl)cc2Cl)=C3)ccc1N1CCNCC1] | 6-(2,4-dichlorophenyl)-N-(3- fluoro-4-(piperazin-1- yl)phenyl)-8,9- dihydroimidazo[1′,2′:1,6]pyrido [2,3-d]pyrimidin-2-amine |
| 2 | 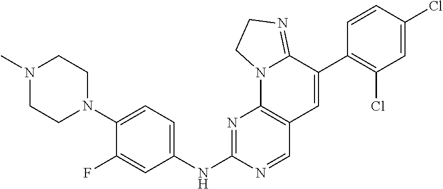 [SMILES: CN1CCN(c2ccc(Nc3ncc4c(n3)N3CCN=C3C(c3ccc(Cl)cc3Cl)=C4)cc2F)CC1] | 6-(2,4-dichlorophenyl)-N-(3- fluoro-4-(4-methylpiperazin- 1-yl)phenyl)-8,9- dihydroimidazo[1′,2′:1,6]pyrido [2,3-d]pyrimidin-2-amine |
| 3 | 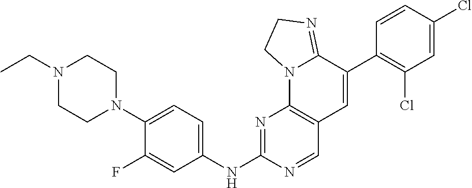 [SMILES: CCN1CCN(c2ccc(Nc3ncc4c(n3)N3CCN=C3C(c3ccc(Cl)cc3Cl)=C4)cc2F)CC1] | 6-(2,4-dichlorophenyl)-N-(4- (4-ethylpiperazin-1-yl)-3- fluorophenyl)-8,9- dihydroimidazo[1′,2′:1,6]pyrido [2,3-d]pyrimidin-2-amine |
| 4 | 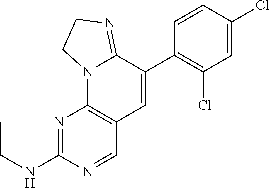 [SMILES: CCNc1ncc2c(n1)N1CCN=C1C(c1ccc(Cl)cc1Cl)=C2] | 6-(2,4-dichlorophenyl)-N- ethyl-8,9- dihydroimidazo[1′,2′:1,6]pyrido [2,3-d]pyrimidin-2-amine |
| 5 | 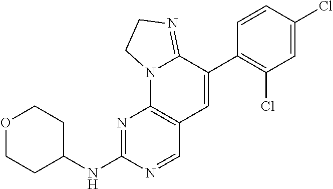 [SMILES: Clc1ccc(C2=Cc3cnc(NC4CCOCC4)nc3N3CCN=C23)c(Cl)c1] | 6-(2,4-dichlorophenyl)-N- (tetrahydro-2H-pyran-4-yl)- 8,9- dihydroimidazo[1′,2′:1,6]pyrido [2,3-d]pyrimidin-2-amine |
| 6 | 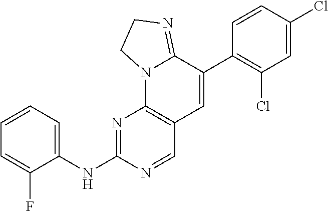 [SMILES: Fc1ccccc1Nc1ncc2c(n1)N1CCN=C1C(c1ccc(Cl)cc1Cl)=C2] | 6-(2,4-dichlorophenyl)-N-(2- fluorophenyl)-8,9- dihydroimidazo[1′,2′:1,6]pyrido [2,3-d]pyrimidin-2-amine |
| 7 | 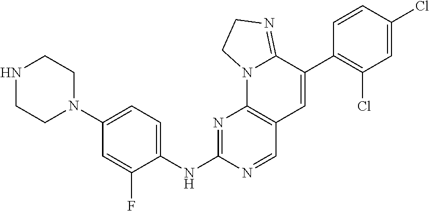 [SMILES: Fc1cc(N2CCNCC2)ccc1Nc1ncc2c(n1)N1CCN=C1C(c1ccc(Cl)cc1Cl)=C2] | 6-(2,4-dichlorophenyl)-N-(2- fluoro-4-(piperazin-1- yl)phenyl)-8,9- dihydroimidazo[1′,2′:1,6]pyrido [2,3-d]pyrimidin-2-amine |
| 8 | 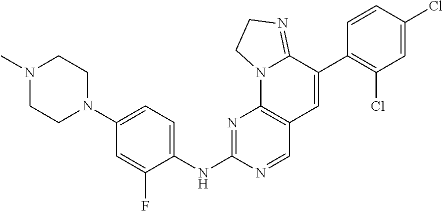 [SMILES: CN1CCN(c2ccc(Nc3ncc4c(n3)N3CCN=C3C(c3ccc(Cl)cc3Cl)=C4)c(F)c2)CC1] | 6-(2,4-dichlorophenyl)-N-(2- fluoro-4-(4-methylpiperazin- 1-yl)phenyl)-8,9- dihydroimidazo[1′,2′:1,6]pyrido [2,3-d]pyrimidin-2-amine |
| 9 | 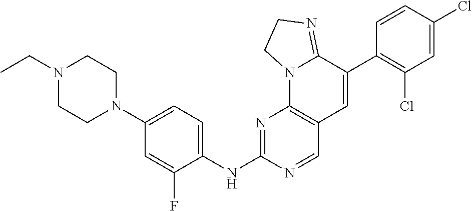 [SMILES: CCN1CCN(c2ccc(Nc3ncc4c(n3)N3CCN=C3C(c3ccc(Cl)cc3Cl)=C4)c(F)c2)CC1] | 6-(2,4-dichlorophenyl)-N-(4- (4-ethylpiperazin-1-yl)-2- fluorophenyl)-8,9- dihydroimidazo[1′,2′:1,6]pyrido [2,3-d]pyrimidin-2-amine |
| 10 | 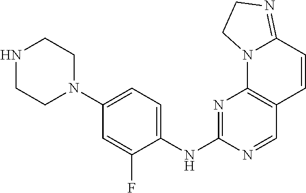 [SMILES: Fc1cc(N2CCNCC2)ccc1Nc1ncc2c(n1)N1CCN=C1C=C2] | N-(2-fluoro-4-(piperazin-1- yl)phenyl)-8,9- dihydroimidazo[1′,2′:1,6]pyrido [2,3-d]pyrimidin-2-amine |
| 11 | 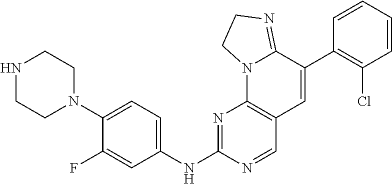 [SMILES: Fc1cc(Nc2ncc3c(n2)N2CCN=C2C(c2ccccc2Cl)=C3)ccc1N1CCNCC1] | 6-(2-chlorophenyl)-N-(3- fluoro-4-(piperazin-1- yl)phenyl)-8,9- dihydroimidazo[1′,2′:1,6]pyrido [2,3-d]pyrimidin-2-amine |
| 12 | 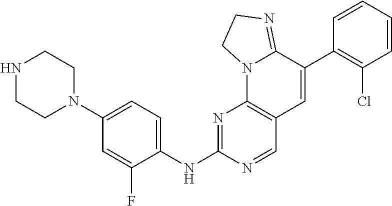 [SMILES: Fc1cc(N2CCNCC2)ccc1Nc1ncc2c(n1)N1CCN=C1C(c1ccccc1Cl)=C2] | 6-(2-chlorophenyl)-N-(2- fluoro-4-(piperazin-1- yl)phenyl)-8,9- dihydroimidazo[1′,2′:1,6]pyrido [2,3-d]pyrimidin-2-amine |
| 13 | 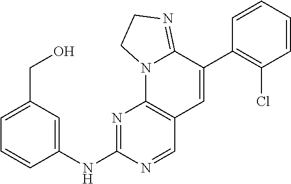 [SMILES: OCc1cccc(Nc2ncc3c(n2)N2CCN=C2C(c2ccccc2Cl)=C3)c1] | (3-((6-(2-chlorophenyl)-8,9- dihydroimidazo[1′,2′:1,6]pyrido [2,3-d]pyrimidin-2- yl)amino)phenyl)methanol |
| 14 | 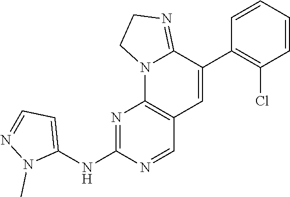 [SMILES: Cn1nccc1Nc1ncc2c(n1)N1CCN=C1C(c1ccccc1Cl)=C2] | 6-(2-chlorophenyl)-N-(1- methyl-1H-pyrazol-5-yl)-8,9- dihydroimidazo[1′,2′:1,6]pyrido [2,3-d]pyrimidin-2-amine |
| 15 | 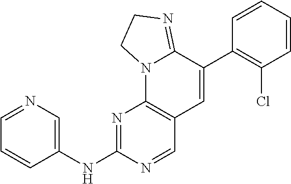 [SMILES: Clc1ccccc1C1=Cc2cnc(Nc3cccnc3)nc2N2CCN=C12] | 6-(2-chlorophenyl)-N- (pyridin-3-yl)-8,9- dihydroimidazo[1′,2′:1,6]pyrido [2,3-d]pyrimidin-2-amine |
| 16 | 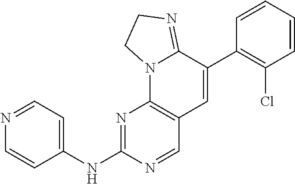 [SMILES: Clc1ccccc1C1=Cc2cnc(Nc3ccncc3)nc2N2CCN=C12] | 6-(2-chlorophenyl)-N- (pyridin-4-yl)-8,9- dihydroimidazo[1′,2′:1,6]pyrido [2,3-d]pyrimidin-2-amine |
| 17 | 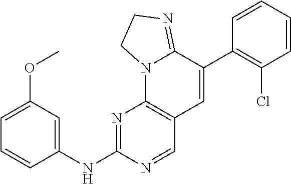 [SMILES: COc1cccc(Nc2ncc3c(n2)N2CCN=C2C(c2ccccc2Cl)=C3)c1] | 6-(2-chlorophenyl)-N-(3- methoxyphenyl)-8,9- dihydroimidazo[1′,2′:1,6]pyrido [2,3-d]pyrimidin-2-amine |
| 18 | 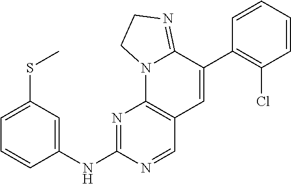 [SMILES: CSc1cccc(Nc2ncc3c(n2)N2CCN=C2C(c2ccccc2Cl)=C3)c1] | 6-(2-chlorophenyl)-N-(3- (methylthio)phenyl)-8,9- dihydroimidazo[1′,2′:1,6]pyrido [2,3-d]pyrimidin-2-amine |
| 19 | 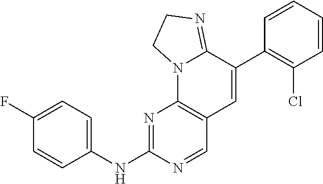 [SMILES: Fc1ccc(Nc2ncc3c(n2)N2CCN=C2C(c2ccccc2Cl)=C3)cc1] | 6-(2-chlorophenyl)-N-(4- fluorophenyl)-8,9- dihydroimidazo[1′,2′:1,6]pyrido [2,3-d]pyrimidin-2-amine |
| 20 | 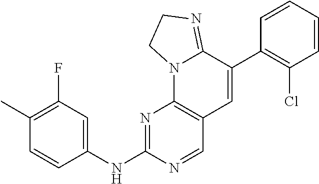 [SMILES: Cc1ccc(Nc2ncc3c(n2)N2CCN=C2C(c2ccccc2Cl)=C3)cc1F] | 6-(2-chlorophenyl)-N-(3- fluoro-4-methylphenyl)-8,9- dihydroimidazo[1′,2′:1,6]pyrido [2,3-d]pyrimidin-2-amine |
| 21 | 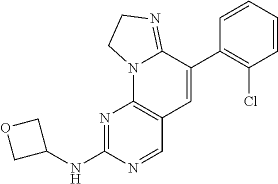 [SMILES: Clc1ccccc1C1=Cc2cnc(NC3COC3)nc2N2CCN=C12] | 6-(2-chlorophenyl)-N- (oxetan-3-yl)-8,9- dihydroimidazo[1′,2′:1,6]pyrido [2,3-d]pyrimidin-2-amine |
| 22 | 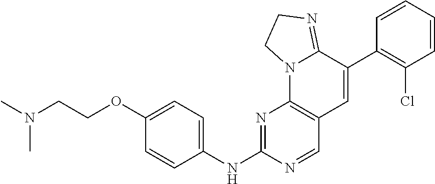 [SMILES: CN(C)CCOc1ccc(Nc2ncc3c(n2)N2CCN=C2C(c2ccccc2Cl)=C3)cc1] | 6-(2-chlorophenyl)-N-(4-(2- (dimethylamino)ethoxy) phenyl)-8,9- dihydroimidazo[1′,2′:1,6]pyrido [2,3-d]pyrimidin-2-amine |
| 23 | 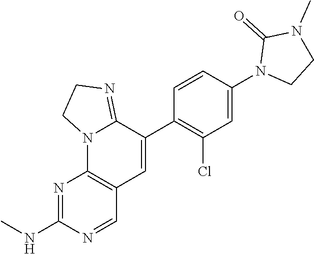 [SMILES: CNc1ncc2c(n1)N1CCN=C1C(c1ccc(N3CCN(C)C3=O)cc1Cl)=C2] | 1-(3-chloro-4-(2- (methylamino)-8,9- dihydroimidazo[1′,2′:1,6]pyrido [2,3-d]pyrimidin-6- yl)phenyl)-3- methylimidazolidin-2-one |
| 24 | 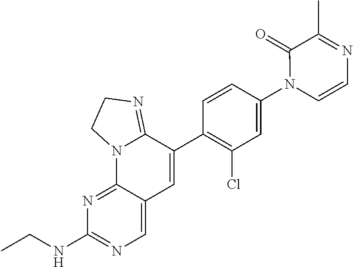 [SMILES: CCNc1ncc2c(n1)N1CCN=C1C(c1ccc(-n3ccnc(C)c3=O)cc1Cl)=C2] | 1-(3-chloro-4-(2- (ethylamino)-8,9- dihydroimidazo[1′,2′:1,6]pyrido [2,3-d]pyrimidin-6- yl)phenyl)-3-methylpyrazin- 2(1H)-one |
| 25 | 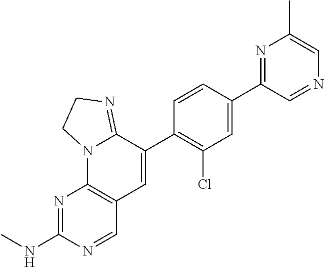 [SMILES: CNc1ncc2c(n1)N1CCN=C1C(c1ccc(-c3cncc(C)n3)cc1Cl)=C2] | 6-(2-chloro-4-(6- methylpyrazin-2-yl)phenyl)- N-methyl-8,9- dihydroimidazo[1′,2′:1,6]pyrido [2,3-d]pyrimidin-2-amine |
| 26 | 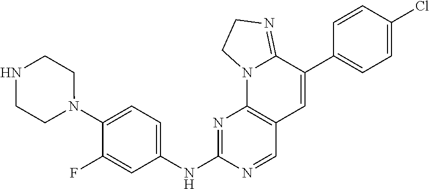 [SMILES: Fc1cc(Nc2ncc3c(n2)N2CCN=C2C(c2ccc(Cl)cc2)=C3)ccc1N1CCNCC1] | 6-(4-chlorophenyl)-N-(3- fluoro-4-(piperazin-1- yl)phenyl)-8,9- dihydroimidazo[1′,2′:1,6]pyrido [2,3-d]pyrimidin-2-amine |
| 27 | 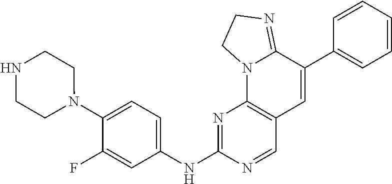 [SMILES: Fc1cc(Nc2ncc3c(n2)N2CCN=C2C(c2ccccc2)=C3)ccc1N1CCNCC1] | N-(3-fluoro-4-(piperazin-1- yl)phenyl)-6-phenyl-8,9- dihydroimidazo[1′,2′:1,6]pyrido [2,3-d]pyrimidin-2-amine |
| 28 | 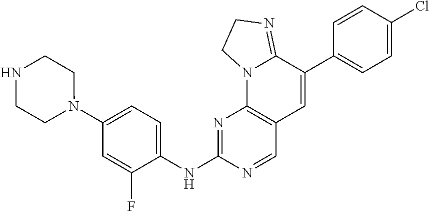 [SMILES: Fc1cc(N2CCNCC2)ccc1Nc1ncc2c(n1)N1CCN=C1C(c1ccc(Cl)cc1)=C2] | 6-(4-chlorophenyl)-N-(2- fluoro-4-(piperazin-1- yl)phenyl)-8,9- dihydroimidazo[1′,2′:1,6]pyrido [2,3-d]pyrimidin-2-amine |
| 29 | 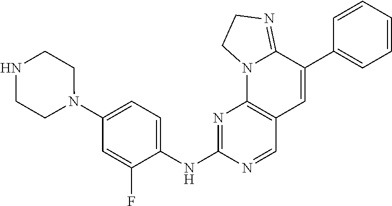 [SMILES: Fc1cc(N2CCNCC2)ccc1Nc1ncc2c(n1)N1CCN=C1C(c1ccccc1)=C2] | N-(2-fluoro-4-(piperazin-1- yl)phenyl)-6-phenyl-8,9- dihydroimidazo[1′,2′:1,6]pyrido [2,3-d]pyrimidin-2-amine |
| 30 | 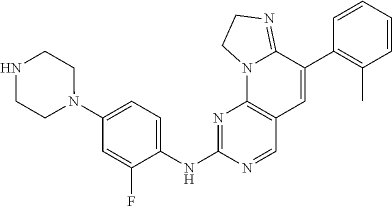 [SMILES: Cc1ccccc1C1=Cc2cnc(Nc3ccc(N4CCNCC4)cc3F)nc2N2CCN=C12] | N-(2-fluoro-4-(piperazin-1- yl)phenyl)-6-(o-tolyl)-8,9- dihydroimidazo[1′,2′:1,6]pyrido [2,3-d]pyrimidin-2-amine |
| 31 | 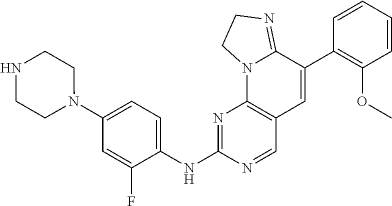 [SMILES: COc1ccccc1C1=Cc2cnc(Nc3ccc(N4CCNCC4)cc3F)nc2N2CCN=C12] | N-(2-fluoro-4-(piperazin-1- yl)phenyl)-6-(2- methoxyphenyl)-8,9- dihydroimidazo[1′,2′:1,6]pyrido [2,3-d]pyrimidin-2-amine |
| 32 | 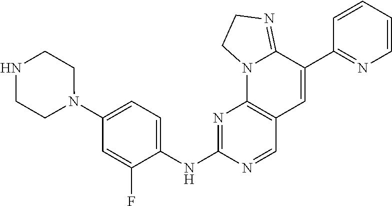 [SMILES: Fc1cc(N2CCNCC2)ccc1Nc1ncc2c(n1)N1CCN=C1C(c1ccccn1)=C2] | N-(2-fluoro-4-(piperazin-1- yl)phenyl)-6-(pyridin-2-yl)- 8,9- dihydroimidazo[1′,2′:1,6]pyrido [2,3-d]pyrimidin-2-amine |
| 33 | 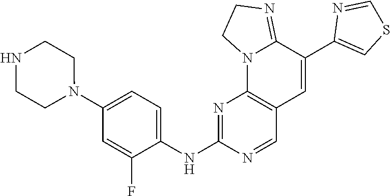 [SMILES: Fc1cc(N2CCNCC2)ccc1Nc1ncc2c(n1)N1CCN=C1C(c1cscn1)=C2] | N-(2-fluoro-4-(piperazin-1- yl)phenyl)-6-(thiazol-4-yl)- 8,9- dihydroimidazo[1′,2′:1,6]pyrido [2,3-d]pyrimidin-2-amine |
| 34 | 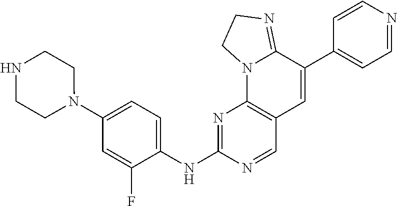 [SMILES: Fc1cc(N2CCNCC2)ccc1Nc1ncc2c(n1)N1CCN=C1C(c1ccncc1)=C2] | N-(2-fluoro-4-(piperazin-1- yl)phenyl)-6-(pyridin-4-yl)- 8,9- dihydroimidazo[1′,2′:1,6]pyrido [2,3-d]pyrimidin-2-amine |
| 35 | 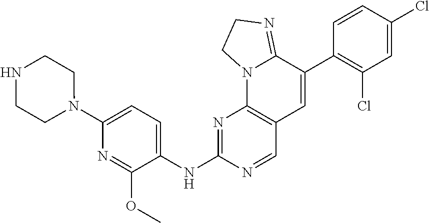 [SMILES: COc1nc(N2CCNCC2)ccc1Nc1ncc2c(n1)N1CCN=C1C(c1ccc(Cl)cc1Cl)=C2] | 6-(2,4-dichlorophenyl)-N-(2- methoxy-6-(piperazin-1- yl)pyridin-3-yl)-8,9- dihydroimidazo[1′,2′:1,6]pyrido [2,3-d]pyrimidin-2-amine |
| 36 | 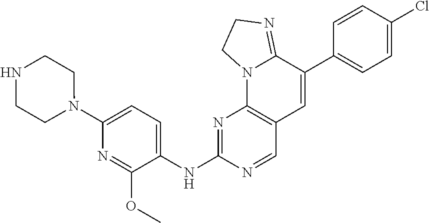 [SMILES: COc1nc(N2CCNCC2)ccc1Nc1ncc2c(n1)N1CCN=C1C(c1ccc(Cl)cc1)=C2] | 6-(4-chlorophenyl)-N-(2- methoxy-6-(piperazin-1- yl)pyridin-3-yl)-8,9- dihydroimidazo[1′,2′:1,6]pyrido [2,3-d]pyrimidin-2-amine |
| 37 | 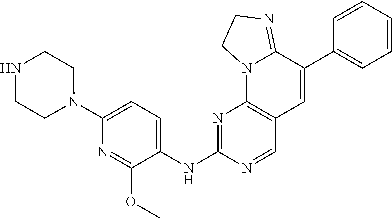 [SMILES: COc1nc(N2CCNCC2)ccc1Nc1ncc2c(n1)N1CCN=C1C(c1ccccc1)=C2] | N-(2-methoxy-6-(piperazin-1- yl)pyridin-3-yl)-6-phenyl-8,9- dihydroimidazo[1′,2′:1,6]pyrido [2,3-d]pyrimidin-2-amine |
| 38 | 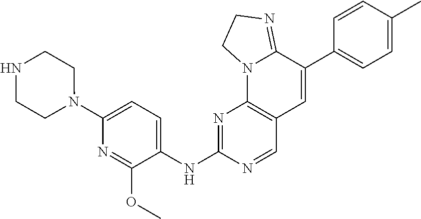 [SMILES: COc1nc(N2CCNCC2)ccc1Nc1ncc2c(n1)N1CCN=C1C(c1ccc(C)cc1)=C2] | N-(2-methoxy-6-(piperazin-1- yl)pyridin-3-yl)-6-(p-tolyl)- 8,9- dihydroimidazo[1′,2′:1,6]pyrido [2,3-d]pyrimidin-2-amine |
| 39 | 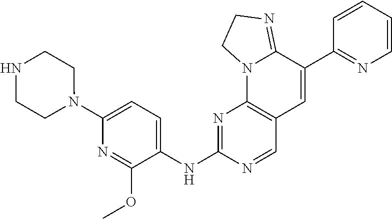 [SMILES: COc1nc(N2CCNCC2)ccc1Nc1ncc2c(n1)N1CCN=C1C(c1ccccn1)=C2] | N-(2-methoxy-6-(piperazin-1- yl)pyridin-3-yl)-6-(pyridin-2- yl)-8,9- dihydroimidazo[1′,2′:1,6]pyrido [2,3-d]pyrimidin-2-amine |
| 40 | 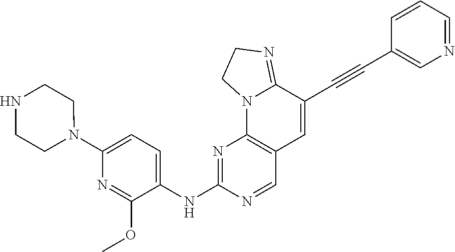 [SMILES: COc1nc(N2CCNCC2)ccc1Nc1ncc2c(n1)N1CCN=C1C(C#Cc1cccnc1)=C2] | N-(2-methoxy-6-(piperazin-1- yl)pyridin-3-yl)-6-(pyridin-3- ylethynyl)-8,9- dihydroimidazo[1′,2′:1,6]pyrido [2,3-d]pyrimidin-2-amine |
| 41 | 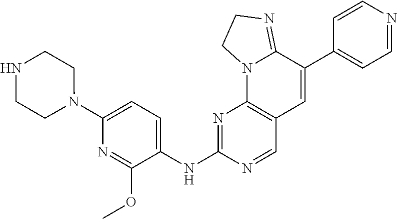 [SMILES: COc1nc(N2CCNCC2)ccc1Nc1ncc2c(n1)N1CCN=C1C(c1ccncc1)=C2] | N-(2-methoxy-6-(piperazin-1- yl)pyridin-3-yl)-6-(pyridin-4- yl)-8,9- dihydroimidazo[1′,2′:1,6]pyrido [2,3-d]pyrimidin-2-amine |
| 42 | 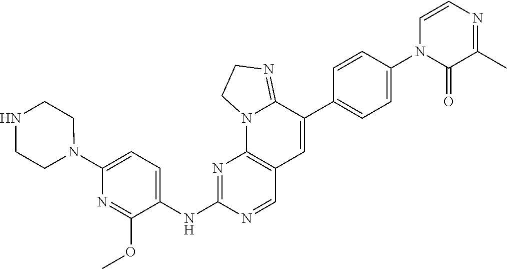 [SMILES: COc1nc(N2CCNCC2)ccc1Nc1ncc2c(n1)N1CCN=C1C(c1ccc(-n3ccnc(C)c3=O)cc1)=C2] | 1-(4-(2-((2-methoxy-6- (piperazin-1-yl)pyridin-3- yl)amino)-8,9- dihydroimidazo[1′,2′:1,6]pyrido [2,3-d]pyrimidin-6- yl)phenyl)-3-methylpyrazin- 2(1H)-one |
| 43 | 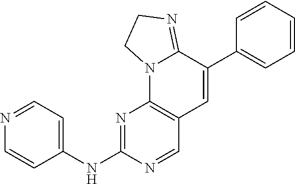 [SMILES: C1=C(c2ccccc2)C2=NCCN2c2nc(Nc3ccncc3)ncc21] | 6-phenyl-N-(pyridin-4-yl)- 8,9- dihydroimidazo[1′,2′:1,6]pyrido [2,3-d]pyrimidin-2-amine |
| 44 | 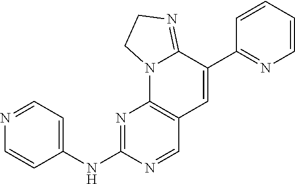 [SMILES: C1=C(c2ccccn2)C2=NCCN2c2nc(Nc3ccncc3)ncc21] | 6-(pyridin-2-yl)-N-(pyridin-4- yl)-8,9- dihydroimidazo[1′,2′:1,6]pyrido [2,3-d]pyrimidin-2-amine |
| 45 | 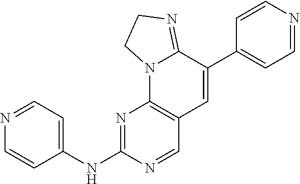 [SMILES: C1=C(c2ccncc2)C2=NCCN2c2nc(Nc3ccncc3)ncc21] | N,6-di(pyridin-4-yl)-8,9- dihydroimidazo[1′,2′:1,6]pyrido [2,3-d]pyrimidin-2-amine |
| 46 | 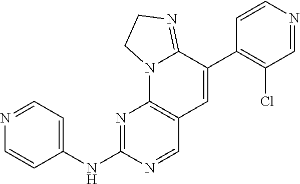 [SMILES: Clc1cnccc1C1=Cc2cnc(Nc3ccncc3)nc2N2CCN=C12] | 6-(3-chloropyridin-4-yl)-N- (pyridin-4-yl)-8,9- dihydroimidazo[1′,2′:1,6]pyrido [2,3-d]pyrimidin-2-amine |
| 47 | 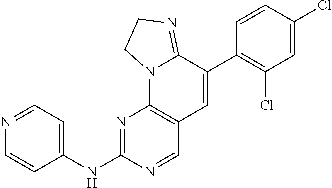 [SMILES: Clc1ccc(C2=Cc3cnc(Nc4ccncc4)nc3N3CCN=C23)c(Cl)c1] | 6-(2,4-dichlorophenyl)-N- (pyridin-4-yl)-8,9- dihydroimidazo[1′,2′:1,6]pyrido [2,3-d]pyrimidin-2-amine |
| 48 | 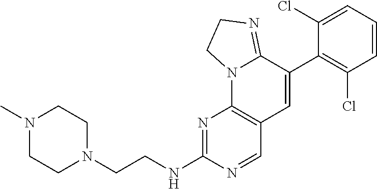 [SMILES: CN1CCN(CCNc2ncc3c(n2)N2CCN=C2C(c2c(Cl)cccc2Cl)=C3)CC1] | 6-(2,6-dichlorophenyl)-N-(2- (4-methylpiperazin-1- yl)ethyl)-8,9- dihydroimidazo[1′,2′:1,6]pyrido [2,3-d]pyrimidin-2-amine |
| 49 | 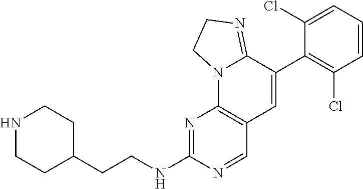 [SMILES: Clc1cccc(Cl)c1C1=Cc2cnc(NCCC3CCNCC3)nc2N2CCN=C12] | 6-(2,6-dichlorophenyl)-N-(2- (piperidin-4-yl)ethyl)-8,9- dihydroimidazo[1′,2′:1,6]pyrido [2,3-d]pyrimidin-2-amine |
| 50 | 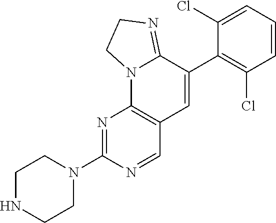 [SMILES: Clc1cccc(Cl)c1C1=Cc2cnc(N3CCNCC3)nc2N2CCN=C12] | 6-(2,6-dichlorophenyl)-2- (piperazin-1-yl)-8,9- dihydroimidazo[1′,2′:1,6]pyrido [2,3-d]pyrimidine |
| 51 | 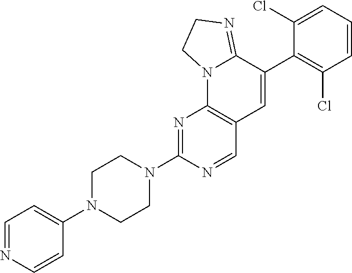 [SMILES: Clc1cccc(Cl)c1C1=Cc2cnc(N3CCN(c4ccncc4)CC3)nc2N2CCN=C12] | 6-(2,6-dichlorophenyl)-2-(4- (pyridin-4-yl)piperazin-1-yl)- 8,9- dihydroimidazo[1′,2′:1,6]pyrido [2,3-d]pyrimidine |
| 52 | 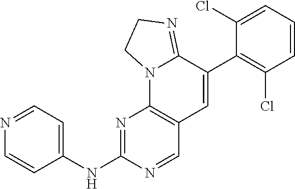 [SMILES: Clc1cccc(Cl)c1C1=Cc2cnc(Nc3ccncc3)nc2N2CCN=C12] | 6-(2,6-dichlorophenyl)-N- (pyridin-4-yl)-8,9- dihydroimidazo[1′,2′:1,6]pyrido [2,3-d]pyrimidin-2-amine |
| 53 | 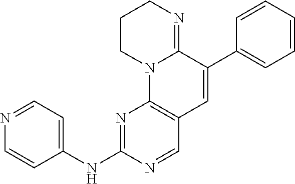 [SMILES: C1=C(c2ccccc2)C2=NCCCN2c2nc(Nc3ccncc3)ncc21] | 6-phenyl-N-(pyridin-4-yl)- 9,10-dihydro-8H-pyrido[1,6- a:2,3-d′]dipyrimidin-2-amine |
| 54 | 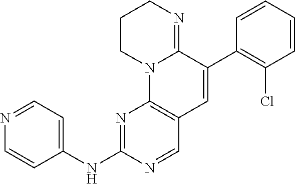 [SMILES: Clc1ccccc1C1=Cc2cnc(Nc3ccncc3)nc2N2CCCN=C12] | 6-(2-chlorophenyl)-N- (pyridin-4-yl)-9,10-dihydro- 8H-pyrido[1,6-a:2,3- d′]dipyrimidin-2-amine |
| 55 | 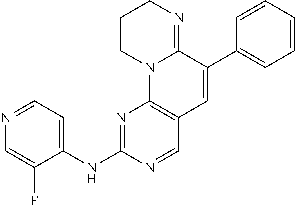 [SMILES: Fc1cnccc1Nc1ncc2c(n1)N1CCCN=C1C(c1ccccc1)=C2] | N-(3-fluoropyridin-4-yl)-6- phenyl-9,10-dihydro-8H- pyrido[1,6-a:2,3- d′]dipyrimidin-2-amine |
| 56 | 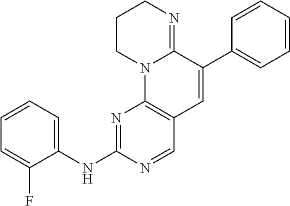 [SMILES: Fc1ccccc1Nc1ncc2c(n1)N1CCCN=C1C(c1ccccc1)=C2] | N-(2-fluorophenyl)-6-phenyl- 9,10-dihydro-8H-pyrido[1,6- a:2,3-d′]dipyrimidin-2-amine |
| 57 | 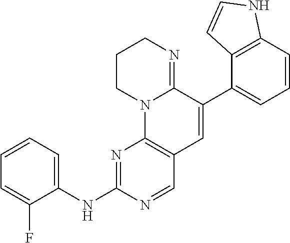 [SMILES: Fc1ccccc1Nc1ncc2c(n1)N1CCCN=C1C(c1cccc3[nH]ccc13)=C2] | N-(2-fluorophenyl)-6-(1H- indol-4-yl)-9,10-dihydro-8H- pyrido[1,6-a:2,3- d′]dipyrimidin-2-amine |
| 58 | 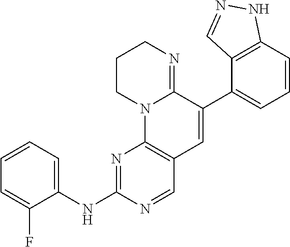 [SMILES: Fc1ccccc1Nc1ncc2c(n1)N1CCCN=C1C(c1cccc3[nH]ncc13)=C2] | N-(2-fluorophenyl)-6-(1H- indazol-4-yl)-9,10-dihydro- 8H-pyrido[1,6-a:2,3- d′]dipyrimidin-2-amine |
| 59 | 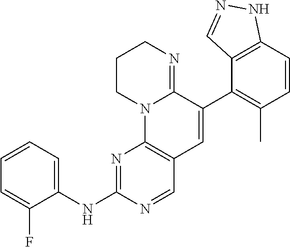 [SMILES: Cc1ccc2[nH]ncc2c1C1=Cc2cnc(Nc3ccccc3F)nc2N2CCCN=C12] | N-(2-fluorophenyl)-6-(5- methyl-1H-indazol-4-yl)- 9,10-dihydro-8H-pyrido[1,6- a:2,3-d′]dipyrimidin-2-amine |
| 60 | 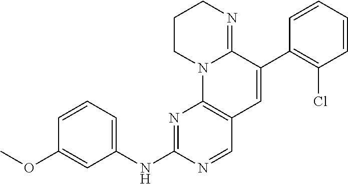 [SMILES: COc1cccc(Nc2ncc3c(n2)N2CCCN=C2C(c2ccccc2Cl)=C3)c1] | 6-(2-chlorophenyl)-N-(3- methoxyphenyl)-9,10- dihydro-8H-pyrido[1,6-a:2,3- d′]dipyrimidin-2-amine |
| 61 | 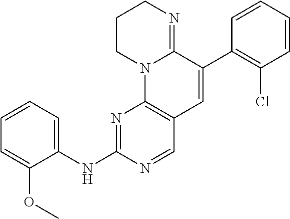 [SMILES: COc1ccccc1Nc1ncc2c(n1)N1CCCN=C1C(c1ccccc1Cl)=C2] | 6-(2-chlorophenyl)-N-(2- methoxyphenyl)-9,10- dihydro-8H-pyrido[1,6-a:2,3- d′]dipyrimidin-2-amine |
| 62 | 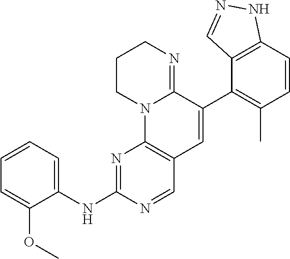 [SMILES: COc1ccccc1Nc1ncc2c(n1)N1CCCN=C1C(c1c(C)ccc3[nH]ncc13)=C2] | N-(2-methoxyphenyl)-6-(5- methyl-1H-indazol-4-yl)- 9,10-dihydro-8H-pyrido[1,6- a:2,3-d′]dipyrimidin-2-amine |
| 63 | 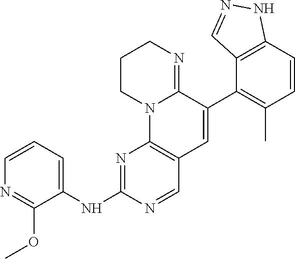 [SMILES: COc1ncccc1Nc1ncc2c(n1)N1CCCN=C1C(c1c(C)ccc3[nH]ncc13)=C2] | N-(2-methoxypyridin-3-yl)-6- (5-methyl-1H-indazol-4-yl)- 9,10-dihydro-8H-pyrido[1,6- a:2,3-d′]dipyrimidin-2-amine |
| 64 | 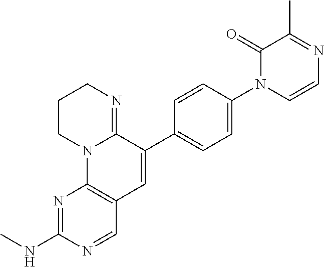 [SMILES: CNc1ncc2c(n1)N1CCCN=C1C(c1ccc(-n3ccnc(C)c3=O)cc1)=C2] | 3-methyl-1-(4-(2- (methylamino)-9,10-dihydro- 8H-pyrido[1,6-a:2,3- d′]dipyrimidin-6- yl)phenyl)pyrazin-2(1H)-one |
| 65 | 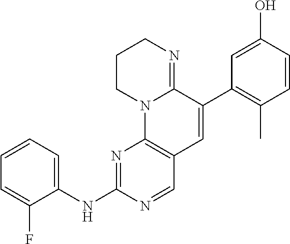 [SMILES: Cc1ccc(O)cc1C1=Cc2cnc(Nc3ccccc3F)nc2N2CCCN=C12] | 3-(2-((2-fluorophenyl)amino)- 9,10-dihydro-8H-pyrido[1,6- a:2,3-d′]dipyrimidin-6-yl)-4- methylphenol |
| 66 | 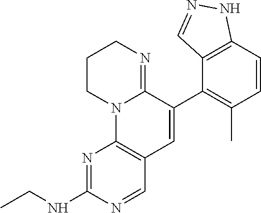 [SMILES: CCNc1ncc2c(n1)N1CCCN=C1C(c1c(C)ccc3[nH]ncc13)=C2] | N-ethyl-6-(5-methyl-1H- indazol-4-yl)-9,10-dihydro- 8H-pyrido[1,6-a:2,3- d′]dipyrimidin-2-amine |
| 67 | 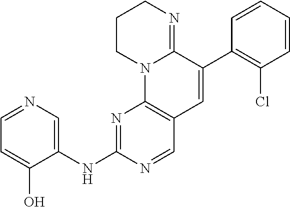 [SMILES: Oc1ccncc1Nc1ncc2c(n1)N1CCCN=C1C(c1ccccc1Cl)=C2] | 3-((6-(2-chlorophenyl)-9,10- dihydro-8H-pyrido[1,6-a:2,3- d′]dipyrimidin-2- yl)amino)pyridin-4-ol |
| 68 | 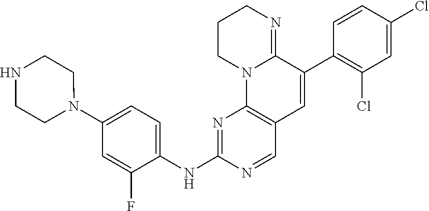 [SMILES: Fc1cc(N2CCNCC2)ccc1Nc1ncc2c(n1)N1CCCN=C1C(c1ccc(Cl)cc1Cl)=C2] | 6-(2,4-dichlorophenyl)-N-(2- fluoro-4-(piperazin-1- yl)phenyl)-9,10-dihydro-8H- pyrido[1,6-a:2,3- d′]dipyrimidin-2-amine |
| 69 | 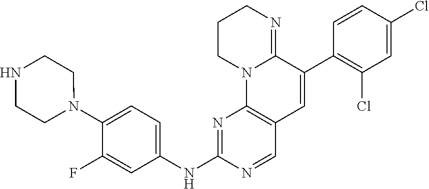 [SMILES: Fc1cc(Nc2ncc3c(n2)N2CCCN=C2C(c2ccc(Cl)cc2Cl)=C3)ccc1N1CCNCC1] | 6-(2,4-dichlorophenyl)-N-(3- fluoro-4-(piperazin-1- yl)phenyl)-9,10-dihydro-8H- pyrido[1,6-a:2,3- d′]dipyrimidin-2-amine |
| 70 | 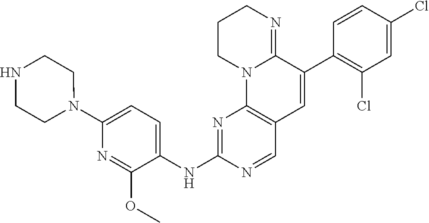 [SMILES: COc1nc(N2CCNCC2)ccc1Nc1ncc2c(n1)N1CCCN=C1C(c1ccc(Cl)cc1Cl)=C2] | 6-(2,4-dichlorophenyl)-N-(2- methoxy-6-(piperazin-1- yl)pyridin-3-yl)-9,10-dihydro- 8H-pyrido[1,6-a:2,3- d′]dipyrimidin-2-amine |
| 71 | 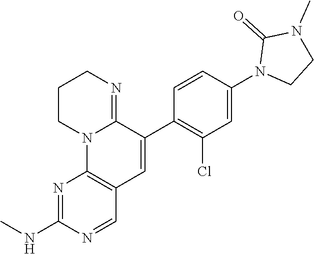 [SMILES: CNc1ncc2c(n1)N1CCCN=C1C(c1ccc(N3CCN(C)C3=O)cc1Cl)=C2] | 1-(3-chloro-4-(2- (methylamino)-9,10-dihydro- 8H-pyrido[1,6-a:2,3- d′]dipyrimidin-6-yl)phenyl)- 3-methylimidazolidin-2-one |
| 72 | 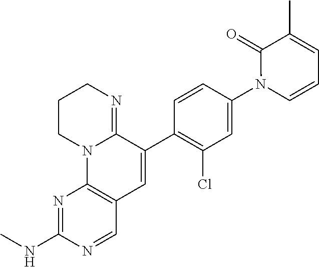 [SMILES: CNc1ncc2c(n1)N1CCCN=C1C(c1ccc(-n3cccc(C)c3=O)cc1Cl)=C2] | 1-(3-chloro-4-(2- (methylamino)-9,10-dihydro- 8H-pyrido[1,6-a:2,3- d′]dipyrimidin-6-yl)phenyl)- 3-methylpyridin-2(1H)-one |
| 73 | 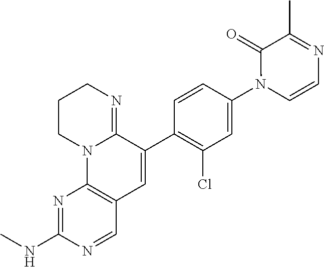 [SMILES: CNc1ncc2c(n1)N1CCCN=C1C(c1ccc(-n3ccnc(C)c3=O)cc1Cl)=C2] | 1-(3-chloro-4-(2- (methylamino)-9,10-dihydro- 8H-pyrido[1,6-a:2,3- d′]dipyrimidin-6-yl)phenyl)- 3-methylpyrazin-2(1H)-one |
| 74 | 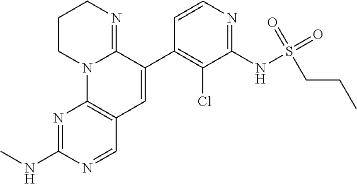 [SMILES: CCCS(=O)(=O)Nc1nccc(C2=Cc3cnc(NC)nc3N3CCCN=C23)c1Cl] | N-(3-chloro-4-(2- (methylamino)-9,10-dihydro- 8H-pyrido[1,6-a:2,3- d′]dipyrimidin-6-yl)pyridin-2- yl)propane-1-sulfonamide |
| 75 | 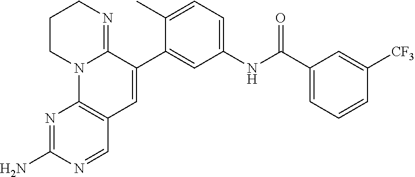 [SMILES: Cc1ccc(NC(=O)c2cccc(C(F)(F)F)c2)cc1C1=Cc2cnc(N)nc2N2CCCN=C12] | N-(3-(2-amino-9,10-dihydro- 8H-pyrido[1,6-a:2,3- d′]dipyrimidin-6-yl)-4- methylphenyl)-3- (trifluoromethyl)benzamide |
| 76 | 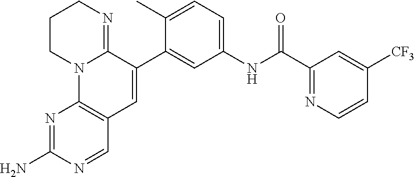 [SMILES: Cc1ccc(NC(=O)c2cc(C(F)(F)F)ccn2)cc1C1=Cc2cnc(N)nc2N2CCCN=C12] | N-(3-(2-amino-9,10-dihydro- 8H-pyrido[1,6-a:2,3- d′]dipyrimidin-6-yl)-4- methylphenyl)-4- (trifluoromethyl)picolinamide |
| 77 | 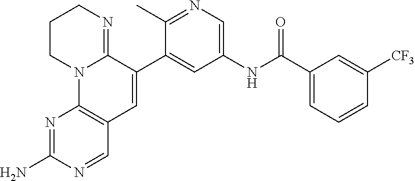 [SMILES: Cc1ncc(NC(=O)c2cccc(C(F)(F)F)c2)cc1C1=Cc2cnc(N)nc2N2CCCN=C12] | N-(5-(2-amino-9,10-dihydro- 8H-pyrido[1,6-a:2,3- d′]dipyrimidin-6-yl)-6- methylpyridin-3-yl)-3- (trifluoromethyl)benzamide |
| 78 | 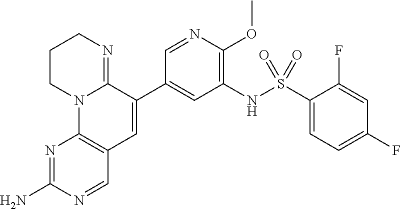 [SMILES: COc1ncc(C2=Cc3cnc(N)nc3N3CCCN=C23)cc1NS(=O)(=O)c1ccc(F)cc1F] | N-(5-(2-amino-9,10-dihydro- 8H-pyrido[1,6-a:2,3- d′]dipyrimidin-6-yl)-2- methoxypyridin-3-yl)-2,4- difluorobenzenesulfonamide |
| 79 | 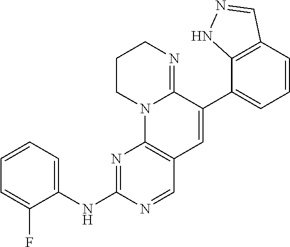 [SMILES: Fc1ccccc1Nc1ncc2c(n1)N1CCCN=C1C(c1cccc3cn[nH]c13)=C2] | N-(2-fluorophenyl)-6-(1H- indazol-7-yl)-9,10-dihydro- 8H-pyrido[1,6-a:2,3- d′]dipyrimidin-2-amine |
| 80 | 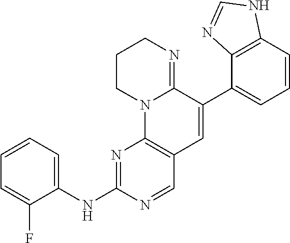 [SMILES: Fc1ccccc1Nc1ncc2c(n1)N1CCCN=C1C(c1cccc3[nH]cnc13)=C2] | 6-(1H-benzo[d]imidazol-4- yl)-N-(2-fluorophenyl)-9,10- dihydro-8H-pyrido[1,6-a:2,3- d′]dipyrimidin-2-amine |
| 81 | 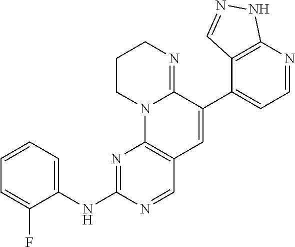 [SMILES: Fc1ccccc1Nc1ncc2c(n1)N1CCCN=C1C(c1ccnc3[nH]ncc13)=C2] | N-(2-fluorophenyl)-6-(1H- pyrazolo[3,4-b]pyridin-4-yl)- 9,10-dihydro-8H-pyrido[1,6- a:2,3-d′]dipyrimidin-2-amine |

or a pharmaceutically acceptable salt, solvate or isotopic derivative thereof.

32. A compound selected from a compound of Table 1 or Table 2, or a pharmaceutically acceptable salt, solvate or isotopic derivative thereof.

33. A pharmaceutical composition comprising a compound of any one of claims 1-32, or a pharmaceutically acceptable salt thereof, and a pharmaceutically acceptable diluent or carrier.

34. A combination comprising a compound of any of claims 1-32, or a pharmaceutically acceptable salt thereof, and a second prophylactic or therapeutic agent.

35. A compound according to any one of claims 1-32 for use in treating and/or preventing a proliferation disorder, such as a cancer, or a tumor in a subject.

36. The compound of claim 35, wherein the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

37. A method for treating and/or preventing a proliferation disorder, such as a cancer, or a tumor in a subject, wherein the method comprises administering to the subject an effective amount of a compound of any one of claims 1-32, or a pharmaceutically acceptable salt thereof, a pharmaceutical composition of claim 33, or a combination of claim 34.

38. The method of claim 37, wherein the proliferation disorder or cancer is selected from the group consisting of malignant or benign tumors of the liver, kidney, bladder, breast, gastric, ovarian, colorectal, prostate, pancreatic, lung, vulval, thyroid, hepatic carcinomas, sarcomas, glioblastomas, head and neck, melanoma, and other hyperplastic conditions such as benign hyperplasia of the skin and benign hyperplasia of the prostate.

39. Use of a compound according to any of claims 1-32 for the manufacture of a medicament.

40. A method for producing an anti-proliferative effect in a subject having a proliferation disorder, a cancer, or a tumor which is sensitive to inhibition of one or more kinases selected from the group consisting of MAPK, PDGFR, Src, PAKs, c-Kit, EphA2, EphB4, FGFR, Axl, and c-Met, comprising administering to the subject an effective amount of a compound of any one of claims 1-32, or a pharmaceutically acceptable salt thereof, or a pharmaceutical composition of claim 33, or a combination of claim 34.

41. A compound according to any one of claims 1-32 for use in the treatment of a neurodegenerative disease.

42. The compound of claim 41, wherein the neurodegenerative disease is selected from the group consisting of Amyotrophic lateral sclerosis, Parkinson's disease, Alzheimer's disease, and Huntington's disease.

43. A method for treating a neurodegenerative disease in a subject, wherein the method comprises administering to the subject an effective amount of a compound of any one of claims 1-32, or a pharmaceutically acceptable salt thereof, a pharmaceutical composition of claim 33, or a combination of claim 34.

44. The method of claim 43, wherein the neurodegenerative disease is selected from the group consist of Amyotrophic lateral sclerosis, Parkinson's disease, Alzheimer's disease, and Huntington's disease.

45. A method for inhibiting an activity of one or more kinases selected from the group consisting of MAPK, PDGFR, Src, PAKs, c-Kit, EphA2, EphB4, FGFR, Axl, and c-Met, comprising contacting the cell with an effective amount of a compound of any one of claims 1-32, or a pharmaceutically acceptable salt thereof, a pharmaceutical composition of claim 33, or a combination of claim 34, wherein the contacting is in vitro, ex vivo, or in vivo.
